(19) United States
(12) Patent Application Publication
(10) Pub. No.: US 2015/0059022 A1
(43) Pub. Date: Feb. 26, 2015

(54) Genes Controlling Plant Root Growth and Development for Stress Tolerance and Method of Their Use

(71) Applicants: Henry T. Nguyen, Columbia, MO (US); Babu Valliyodan, Columbia, MO (US)

(72) Inventors: Henry T. Nguyen, Columbia, MO (US); Babu Valliyodan, Columbia, MO (US)

(73) Assignee: THE CURATORS OF THE UNIVERSITY OF MISSOURI, Columbia, MO (US)

(21) Appl. No.: 14/528,229

(22) Filed: Oct. 30, 2014

Related U.S. Application Data
(62) Division of application No. 12/966,981, filed on Dec. 13, 2010, now abandoned.
Application No. 12/966,981 is a continuation-in-part of application No. 12/138,392, filed on Jun. 12, 2008, now abandoned.
(60) Provisional application No. 61/284,111, filed on Dec. 12, 2009.
(60) Provisional application No. 60/934,321, filed on Jun. 12, 2007.

(51) Int. Cl. C12N 15/82

(52) U.S. Cl. CPC C12N 15/8273; C12N 15/8201; C12N 15/8227; C12N 15/8247; C12N 15/8246; C12N 15/8267
USPC 800/287; 800/298; 800/312; 800/320.1; 800/320.3; 800/320.2; 800/314

(57) ABSTRACT

Microarrays are employed to analyze soybean transcriptome under water stress conditions in different regions of the root at vegetative stage. Drought responsive genes and transcription factors are identified which may be used for enhancing drought tolerance in soybean or other plants through genetic/metabolic engineering. This disclosure pertains to nucleic acid molecules isolated from soybean and maize that encode polypeptides that may be important for drought tolerance. The disclosure also relates to methods of using these genes from soybean in transgenic plants to confer the desired agronomic traits, and to use such genes or regulatory elements thereof to assist germplasm enhancement by molecular breeding or to identify other factors or chemicals that may enhance a plant's capability to grow under drought conditions.

### RELATED APPLICATIONS

This application claims priority to U.S. Provisional Application No. 61/284,111 filed on Dec. 12, 2009. This application is also a continuation-in-part of U.S. patent application Ser. No. 12/138,392 filed on Jun. 12, 2008, which claims priority of U.S. Provisional Application No. 60/934,321 filed on Jun. 12, 2007. The contents of all applications mentioned above are hereby incorporated into this application by reference.

### BACKGROUND

1. Field of the Invention

The present invention relates to methods and materials for identifying genes and the regulatory network in a plant that control the plant's response to various environmental conditions and/or stress. More particularly, the present invention relates to genes in the root of a plant that help confer drought resistance to the plant.

2. Description of the Related Art

Drought is one of the major abiotic stress factors limiting crop productivity worldwide. Global climate changes may further exacerbate the drought situation in major crop-producing countries. Although irrigation may in theory solve the drought problem, it is usually not a viable option because of the cost associated with building and maintaining an effective irrigation system, as well as other non-economical issues, such as the general availability of water (Boyer, 1983). Thus, alternative means for alleviating plant water stress are needed.

In soybean, drought stress during flowering and early pod development significantly increases the rate of flower and pod abortion, thus decreasing final yield (Boyer 1983; Westgate and Peterson 1993). Soybean yield reduction of 40% because of drought is common experience among soybean producers in the United States (Muchow & Sinclair, 1986; Specht et al. 1999).

Mechanisms for selecting drought tolerant plants fall into three general categories. The first is called drought escape, in which selection is aimed at those developmental and maturation traits that match seasonal water availability with crop needs. The second is dehydration avoidance, in which selection is focused on traits that: lessen evaporatory water loss from plant surfaces or maintain water uptake during drought via a deeper and more extensive root system. The last mechanism is dehydration tolerance, in which selection is directed at maintaining cell turgor or enhancing cellular constituents that protect cytoplasmic proteins and membranes from drying.

The molecular mechanisms of abiotic stress responses and the genetic regulatory networks of drought stress tolerance have been reviewed recently (Neumann 2008; Wang et al 2003; Vinocur and Altman 2005; Chaves and Oliveira 2004; Shinozaki et al. 2003). Plant modification for enhanced drought tolerance is mostly based on the manipulation of either transcription and/or signaling factors or genes that directly protect plant cells against water deficit. Despite much progress in the field, understanding the basic biochemical and molecular mechanisms for drought stress perception, transduction, response and tolerance remains a major challenge in the filed. Utilization of the knowledge on drought tolerance to generate plants that can tolerate extreme water deficit condition is a even bigger challenge.

Analysis of changes in gene expression within a target plant is important for revealing the transcriptional regulatory networks. Elucidation of these complex regulatory networks may contribute to our understanding of the responses mounted by a plant to various stresses and developmental changes, which may ultimately lead to crop improvement. DNA microarray assays (Schena et al. 1995; Shalon et al. 1996) have provided an unprecedented opportunity for the generation of gene expression data on a whole-genome scale.

Gene expression profiling using cDNAs or oligonucleotides microarray technology has advanced our understanding of gene regulatory network when a plant is subject to various stresses (Bray 2004; Denby and Gehring 2005). For example, numerous genes that respond to dehydration stress have been identified in Arabidopsis and have been categorized as “rd” (responsive to dehydration) or “erd” (early response to dehydration) (Shinozaki and Yamaguchi-Shinozaki 1999).

There are at least four independent regulatory pathways for gene expression in response to water stress. Out of the four pathways, two are abscisic acid (ABA) dependent and the other two are ABA independent (Shinozaki and Yamaguchi-Shinozaki 2000). In the ABA independent regulatory pathways, a cis-acting element is involved and the Dehydration-responsive element/C-repeat (DRE/CRT) has been identified. DRE/CRT also functions in cold response and high-salt-responsive gene expression. When the DRE/CRT binding protein DREB1/ICBF is overexpressed in a transgenic Arabidopsis plant, changes in expression of more than 40 stress-inducible genes can be observed, which lead to enhanced tolerance to freeze, high salt, and drought (Seki et al, 2001; Fowler and Thomashow 2002; Murayama et al. 2004).

In summary, the production of microarrays and the global transcript profiling of plants have revolutionized the study of gene expression which provides a unique snapshot of how these plants are responding to a particular stress. However, no transcriptional profiling or transcriptome changes have been reported for soybean plants under water stress conditions. A well designed analysis of gene expression in soybean grown under drought may help illuminate the regulatory networks in soybean under these adverse conditions, which may further lead to development of new soybean plant that can better tolerate drought conditions than conventional strains.

### SUMMARY

The instrumentalities described herein overcome the problems outlined above and advance the art by providing genes and DNA regulatory elements in plant roots which may play an important part in response to drought conditions. These genes are collectively called Root Specific Drought Response Genes (RSDRGs) in this disclosure. Methodology is also provided whereby these drought responsive genes may be introduced into a plant to enhance its capability to grow and reproduce under water deficit conditions. The RSDRGs or fragments thereof may be introduced and expressed in the roots of a host plant, or alternatively, they may be expressed in other organs of the host plants. Regulatory elements, such as promoters and enhancers of the RSDRGs, may also be employed to control expression of genes that are not yet identified but may prove beneficial for enhancing a plant's capability to grow under drought conditions.

In crop plants, root growth is often less sensitive to low tissue water potential than shoot growth. This root specific trait confers drought tolerance to some plants to certain degree, but the mechanisms underlying root growth and development under drought stress conditions (low water potential) are not well understood. To discover genes and to identify mechanisms that determine these responses to low water potential conditions, gene expression patterns were profiled from three regions of water-stressed and well-watered roots, respectively. These analyses, along with comparisons with the gene expression patterns in maize root regions under low water potentials with those of soybean, support the notion that there is a distinct and species-specific pattern of gene expression under water deficit conditions in root tissues.

This disclosure provides novel drought responsive genes that may play a role in root growth and development under normal conditions. These candidates may be used for enhanced drought tolerance in soybean and other crops through genetic/metabolic engineering. The genes and methods disclosed herein apply not only to soybean and maize but also to drought stress tolerance/avoidance mechanisms in other plants. This disclosure provides both early and late stage stress responsive genes/proteins controlling root growth and development under water deficits. One or more of these genes or their fragments from soybean or maize as disclosed herein may be expressed in transgenic plants to confer certain agronomic traits to a host plant. The genes may also be used to assist germplasm enhancement by molecular breeding.

More particularly, microarray experiments are conducted to analyze the gene expression pattern in different root regions of soybean plants in response to drought stress. Tissue specific transcriptomes may be compared to help elucidate the transcriptional regulatory network and facilitate the identification of stress specific genes and promoters. For purpose of this disclosure, genes whose expression are either up- or down-regulated in response to drought condition are referred to as Drought Response Genes (or DRGs). Some DRGs may show tissue specific expression patterns in response to drought condition. Those DRGs whose expression are up- or down-regulated in the roots are also referred to as Root Specific Drought Response Genes (“RSDRGs”). For purpose of this disclosure, a “RSDRG protein” refers to a protein encoded by a RSDRG.

The microarray experiments described in this disclosure may not have uncovered all the RSDRGs in all plants, or even in soybean alone, due to the variations in experimental conditions, and more importantly, due to the different gene expressions among different plant species. It is also to be understood that certain RSDRGs disclosed here may have been identified and studied previously; however, regulation of their expression under drought condition or their role in drought response may not have been appreciated in previous studies. Alternatively, some RSDRGs may contain novel coding sequences. Thus, it is an object of the present disclosure to identify known or unknown genes whose expression levels are altered in response to drought condition.

For purpose of this disclosure, if a transgenic plant or a genetically altered plant is able to grow at a similar rate and reproduce at a similar yield when water is limited as compared to the host plant or the parental plant with sufficient water supply, it can be said that the transgenic plant or the genetically altered plant has gained drought resistance (or tolerence), or in other words, that such plant is more drought resistant, or more tolerant to drought (or water deficit) conditions than the host or the parent. In one aspect, the transgenic or altered plant may be able to grow at a similar rate and reproduce at a similar yield when water available to the plant is 20% less, 30% less, 40% less, 50% less, more preferably 60% less, than the water available to the host or parental plant. In order to generate a transgenic plant that is more tolerant to drought condition when compared to a host plant, the expression levels of a protein encoded by an endogenous Root Specific Drought Response Gene (RSDRG) or a fragment thereof may be altered to confer a drought resistant phenotype to the host plant.

For example, the transcription, translation or protein stability of the protein encoded by the RSDRG may be modified so that the levels of this protein are rendered significantly higher than the levels of this protein would otherwise be even under the same drought condition. To this end, either the coding or non-coding regions, or both, of the endogenous RSDRG may be modified.

In another aspect, in order to generate a transgenic plant that is more tolerant to drought condition when compared to a host plant, the method may comprise the steps of: (a) introducing into a plant cell a construct comprising a Root Specific Drought Response Gene (RSDRG) or a fragment thereof encoding a polypeptide; and (b) generating a transgenic plant expressing said polypeptide or a fragment thereof. In one embodiment, the Drought Response Gene or a fragment thereof is derived from a plant that is genetically different from the host plant. In another embodiment, the Drought Response Gene or a fragment thereof is derived from a plant that belongs to the same species as the host plant. For instance, an RSDRG identified in soybean may be introduced into soybean as a transgene to confer upon the host increased capability to grow and/or reproduced under mild to severe drought conditions.

The RSDRGs disclosed here include known genes as well as genes whose functions are not yet fully understood. Nevertheless, both known or unknown RSDRGs may be placed under control of a promoter and be transformed into a host plant in accordance with standard plant transformation protocols. The transgenic plants thus obtained may be tested for the expression of the RSDRGs and their capability to grow and/or reproduce under drought conditions as compared to the original host (or parental) plant.

Although the RSDRGs disclosed herein are identified in soybean, they may be introduced into other plants as transgenes. Examples of such other plants may include but are not limited to corn, wheat, rice, and cotton. In another aspect, homologs in other plant species may be identified by PCR, hybridization or by genome search which may share substantial sequence similarity with the RSDRGs disclosed herein. In a preferred embodiment, such a homolog shares at least 90%, more preferably 98%, or even more preferably 99% sequence identity with a protein encoded by a soybean RSDRG.

It is further an object to identify the non-coding regulatory sequences of the RSDRGs which are called Root Specific Drought Response Regulatory Elements (RSDRREs) for purpose of this disclosure. These RSDRREs may be used to prepare DNA constructs for the expression of genes of interest in a host plant. The RSDRREs or the RSDRGs may also be used to screen for factors or chemicals that may affect the expression of certain RSDRGs by interacting with a RSDRRE. Such factors or chemicals may be used to induce drought responses by activating expression of certain genes in a plant.

In one aspect, the genes of interest may be genes from the transgenic host plant, or they may be from other plants or even from non-plant organisms. The genes of interest may be those identified and listed in this disclosure, or they may be the homologs of these genes.

In one embodiment, the genes of interest (RSDRGs) may be placed under control of the RSDRREs such that their expression may be upregulated under drought condition. This arrangement is particularly useful for those genes of interest that may not be desirable under normal condition, because such genes may be placed under a tightly regulated RSDRRE which only drives the expression of the genes of interest when water deficit condition is sensed by the plant. Under control of such a RSDRRE, expression of the gene of interest may be only detected under drought condition. The RSDRRE can thus be used to regulate a heterologous gene such that expression of the heterologous gene is induced under drought conditions.

It is an object of this disclosure to provide a system and a method for the genetic modification of a plant, to increase the resistance of the plant to adverse conditions such as drought and/or excessive temperatures, compared to an unmodified plant.

It is another object of the present invention to provide a transgenic plant that exhibits increased resistance to adverse conditions such as drought and/or excessive temperatures as compared to an unmodified plant.

It is another object of the present invention to provide a system and method of modifying a plant, to alter the metabolism or development of the plant.

In one embodiment, a gene of interest may be placed under control of a tissue specific promoter such that such gene of interest may be expressed in specific site, for example, the guard cells. The expression of the introduced genes may enhance the capacity of a plant to modulate guard cell activity in response to water stress. For instance, the transgene may help reduce stomatal water loss. In addition, other characteristics such as early maturation of plants may be introduced into plants to help cope with drought condition.

Preferably, the transgene is under control of a promoter, which may be a constitutive or inducible promoter. An inducible promoter is inactive under normal condition, and is activated under certain conditions to drive the expression of the gene under its control. Conditions that may activate a promoter include but are not limited to light, heat, certain nutrients or chemicals, and water conditions. A promoter that is activated under water deficit condition is preferred.

In another aspect, a tissue specific promoter, an organ specific promoter, or a cell-specific promoter may be employed to control the transgene. Despite their different names, these promoters are similar in that they are only activated in certain cell, tissue or organ types. It is to be understood that a gene under control of an inducible promoter, or a promoter specific for certain cells, tissues or organs may have low level of expression even under conditions that are not supposed to activate the promoter, a phenomenon known as “leaky expression” in the field. A promoter can be both inducible and tissue specific. By way of example, a transgene may be placed under control of a guard cell specific promoter such that the gene can be inducibly expressed in the guard cell of the transgenic plant.

In another aspect, the present disclosure provides a method of generating a transgenic plant having an altered stress response or an altered phenotype compared to an unmodified plant. The coding sequences of the genes that are disclosed to be upregulated may be placed under a promoter such that the genes can be expressed in the transgenic plant. The method may contain two steps: (a) introducing into a plant cell capable of being transformed and regenerated into a whole plant a construct comprising, in addition to the DNA sequences required for transformation and selection in plants, an expression construct including the coding sequence of a gene that a operatively linked to a promoter for expressing said DNA sequence; and (b) recovery of a plant which contains the expression construct.

The transgenic plant generated by the methods disclosed above may exhibit an altered trait or stress response. The altered traits may include increased tolerance to extreme temperature, such as heat or cold; or increased tolerance to extreme water condition such as drought or excessive water. The transgenic plant may exhibit one or more altered phenotype that may contribute to the resistance to drought condition. These phenotypes may include, by way of example, early maturation, increased growth rate, increased biomass, or increased lipid content.

In accordance with the disclosed methods, the coding sequence to be introduced in the transgenic plant preferably encodes a peptide having at least 70%, more preferably at least 90%, more preferably at least 98% identity, and even more preferably at least 99% identity to the polypeptide encoded by the RSDRGs disclosed in this application. In an alternative aspect, DNA sequence may be oriented in an antisense direction relative to said promoter within said construct.

In accordance with the methods of the present invention, the promoter is preferably selected from the group consisting of an constitutive promoter, an inducible promoter, a tissue specific promoter, and organ specific promoter, a cell-specific promoter. More preferably the promoter is an inducible promoter for expressing said DNA sequence under water deficit conditions.

In another aspect, the present invention provides a method of identifying whether a plant that has been successfully transformed with a construct, characterized in that the method comprises the steps of: (a) introducing into plant cells capable of being transformed and regenerated into whole plants a construct comprising, in addition to the DNA sequences required for transformation and selection in plants, an expression construct that includes a DNA sequence selected from at least one of the RSDRGs disclosed herein, said DNA sequence may be operatively linked to a promoter for expressing said DNA sequence; (b) regenerating the plant cells into whole plants; and (c) subjecting the plants to a screening process to differentiate between transformed plants and non-transformed plants.

The screening process may involve subjecting the plants to environmental conditions suitable to kill non-transformed plants, retain viability in transformed plants. For instance by growing the plants in a medium or soil that contains certain chemicals, such that only those plants expressing the transgenes can survive. In one particular embodiment, after obtaining a transgenic plant that appear to be expressing the transgene, a functional screening may be carried out by growing the plants under water deficit conditions to select for those that can tolerate such a condition.

In another aspect, the present disclosure provides a kit for generating a transgenic plant having an altered stress response or an altered phenotype compared to an unmodified plant, characterized in that the kit comprises: an expression construct including a DNA sequence selected from at least one of the RSDRGs disclosed herein, said DNA sequence may be operatively linked to an promoter suitable for expressing said DNA sequence in a plant cell.

Preferably the kit further includes targeting means for targeting the activity of the protein expressed from the construct to certain tissues or cells of the plant. Preferably the targeting means comprises an inducible, tissue-specific promoter for specific expression of the DNA sequence within certain tissues of the plant. Alternatively the targeting means may be a signal sequence encoded by said expression construct and may contain a series of amino acids covalently linked to the expressed protein.

In accordance with the kit of the present invention, the DNA sequence may encode a peptide having at least 70%, more preferably at least 90%, more preferably at least 98%, or even 99% identity to the peptide encoded by coding sequences selected from at least one of the RSDRGs disclosed herein. In one aspect, said DNA sequence may be oriented in an antisense direction relative to said promoter within said construct.

### BRIEF DESCRIPTION OF THE DRAWINGS

FIG. 1 illustrates a whole soybean seedling (A) and the root tip regions of the soybean seedling root (B).

FIG. 2 shows the numbers of genes that show differential expression at the same time point 5 hour (A) or 48 hour (B) between different root regions.

FIG. 3 shows the number of genes within the same root region, R1(A) or R2 (B), that are differentially expressed between two time points 5 hr and 48 hr after the plants have been subjected to water deficit conditions.

FIG. 4 shows the number of genes that are only differentially expressed in R1, R2 or both R1 and R2 regions of the root up to 48 hours after the plants are exposed to drought conditions (upper panel), and the number of genes that show differential expression at 5 hours, 48 hours after being exposed to drought conditions, as well as those that show differential expression at both 5 hours and 48 hours (lower panel).

FIG. 5 shows the number genes that are differentially expressed under drought condition only in the R2 region but not in the R3 region.

FIG. 6 presents pie charts illustrating the different functional categories of genes that show differential expression in the R1 (8A and 8B) or R2 (8C and 8D) regions after having been subjected to water deficit conditions for 5 hour (8A and 8C) or 48 hours (8B and 8D).

FIG. 7 shows the different categories of the “root region and stress specific transcripts” (RRSST) by their known or predicted function.

### DETAILED DESCRIPTION

The methods and materials described herein relate to gene expression profiling using microarrays, and follow-up analysis to decode the regulatory network that controls a plant's response to drought conditions. More particularly, drought response is analyzed at the molecular level to identify root specific genes and/or promoters which may be activated under water deficit conditions. The coding sequences of such genes may be introduced into a host plant to obtain transgenic plants that are more tolerant to drought than unmodified plants.

It is to be understood that the materials and methods are taught by way of example, and not by limitation. The disclosed instrumentalities may be broader than the particular methods and materials described herein, which may vary within the skill of the art. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the related art. The following terminology and grammatical variants are used in accordance with the definitions set out below.

The present disclosure provides genes whose expression levels are altered in response to stress conditions in soybean plants using genome-wide microarray (or gene chip) analysis of soybean plants grown under water deficit conditions. Those genes identified using microarray analysis may be subject to validation to confirm that their expression levels are altered under the stress conditions. Validation may be conducted using high throughput two-step qRT-PCR and by the delta delta CT method.

Sequences of those genes that have been validated may be subject to further sequence analysis by comparing their sequences to published sequences of various families of genes or proteins. For instance, some of these RSDRGs may encode proteins with substantial sequence similarity to known transcription factors. These transcription factors may play a role in the stress response by activating the transcription of other genes.

The present disclosure provides a system and a method for expressing a protein that may enhance a host's capability to grow or to survive in an adverse environment characterized by water deficit. Although plants are the most preferred host for purpose of this disclosure, the genetic constructs described herein may be introduced into other eukaryotic organisms, if the traits conferred upon these organisms by the constructs are desirable.

The term “genetically altered plant” or “genetically modified plant” refers to a plant whose genetic make-up has been altered or modified such that the modified plant expresses one or more protein that is not normally expressed by the unmodified plant or is expressed at different time or different tissue of the unmodified plant.

The term “transgenic plant” refers to a host plant into which a gene construct has been introduced. A gene construct, also referred to as a construct, an expression construct, or a DNA construct, generally contains as its components at least a coding sequence and a regulatory sequence. A gene construct typically contains at least on component that is foreign to the host plant. For purpose of this disclosure, all components of a gene construct may be from the host plant, but these components are not arranged in the host in the same manner as they are in the gene construct. A regulatory sequence is a non-coding sequence that typically contribute to the regulation of gene expression, at the transcription or translation levels. It is to be understood that certain segments in the coding sequence may be translated but may be later removed from the functional protein. An example of these segments is the so-called signal peptide, which may facilitate the maturation or localization of the translated protein, but is typically removed once the protein reaches its destination. Examples of a regulatory sequence include but are not limited to a promoter, an enhancer, and certain post-transcriptional regulatory elements.

After its introduction into a host plant, a gene construct may exist separately from the host chromosomes. Preferably, the entire gene construct, or at least part of it, is integrated onto a host chromosome. The integration may be mediated by a recombination event, which may be homologous, or non-homologous recombination. The term “express” or “expression” refers to production of RNAs using DNAs as template through transcription or translation of proteins from RNAs or the combination of both transcription and translation.

A “host cell,” as used herein, refers to a prokaryotic or eukaryotic cell that contains heterologous DNA which has been introduced into the cell by any means, e.g., electroporation, calcium phosphate precipitation, microinjection, transformation, viral infection, and/or the like. A “host plant” is a plant into which a transgene is to be introduced. A “parental plant” is the original plant into which genetic changes are to be introduced in order to create a genetically altered plant.

A “vector” is a composition for facilitating introduction, replication and/or expression of a selected nucleic acid in a cell. Vectors include, for example, plasmids, cosmids, viruses, yeast artificial chromosomes (YACs), etc. A “vector nucleic acid” is a nucleic acid vector into which heterologous nucleic acid is optionally inserted and which can then be introduced into an appropriate host cell. Vectors preferably have one or more origins of replication, and one or more sites into which the recombinant DNA can be inserted. Vectors often have convenient markers by which cells with vectors can be selected from those without. By way of example, a vector may encode a drug resistance gene to facilitate selection of cells that are transformed with the vector. Common vectors include plasmids, phages and other viruses, and “artificial chromosomes.” “Expression vectors” are vectors that comprise elements that provide for or facilitate transcription of nucleic acids which are cloned into the vectors. Such elements may include, for example, promoters and/or enhancers operably coupled to a nucleic acid of interest.

“Plasmids” generally are designated herein by a lower case “p” preceded and/or followed by capital letters and/or numbers, in accordance with standard nomenclatures that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors are well known and readily available to those of skill in the art. Moreover, those of skill readily may construct any number of other plasmids suitable for use as described below. The properties, construction and use of such plasmids, as well as other vectors, is readily apparent to those of ordinary skill upon reading the present disclosure.

When a molecule is identified in or can be isolated from a organism, it can be said that such a molecule is derived from said organism. When two organisms have significant difference in the genetic materials in their respective genomes, these two organisms can be said to be genetically different. For purpose of this disclosure, the term “plant” means a whole plant, a seed, or any organ or tissue of a plant that may potentially develop into a whole plant.

The term “isolated” means that the material is removed from its original environment, such as the native or natural environment if the material is naturally occurring. For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

A “recombinant nucleic acid” is one that is made by recombining nucleic acids, e.g., during cloning, DNA evolution or other procedures. A “recombinant polypeptide” is a polypeptide which is produced by expression of a recombinant nucleic acid. An “amino acid sequence” is a polymer of amino acid residues (a protein, polypeptide, etc.) or a character string representing an amino acid polymer, depending on context. Either the given nucleic acid or the complementary nucleic acid can be determined from any specified polynucleotide sequence.

The terms “nucleic acid,” or “polynucleotide” refer to a deoxyribonucleotide, in the case of DNA, or ribonucleotide in the case of RNA polymer in either single- or double-stranded form, and unless otherwise specified, encompasses known analogues of natural nucleotides that can be incorporated into nucleic acids in a manner similar to naturally occurring nucleotides. A “polynucleotide sequence” is a nucleic acid which is a polymer of nucleotides (A, C, T, U, G, etc. or naturally occurring or artificial nucleotide analogues) or a character string representing a nucleic acid, depending on context. Either the given nucleic acid or the complementary nucleic acid can be determined from any specified polynucleotide sequence.

A “subsequence” or “fragment” is any portion of an entire sequence of a DNA, an RNA or a polypeptide (also referred to as “a protein”) molecule, up to and including the full sequence. Typically a subsequence or fragment comprises less than the full-length sequence, and is sometimes referred to as the “truncated version.”

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. Similarly, nucleic acids and/or nucleic acid sequences are homologous when they are derived, naturally or artificially, from a common ancestral nucleic acid or nucleic acid sequence. The homologous molecules can be termed homologs. For example, any naturally occurring RSDRGs, as described herein, can be modified by any available mutagenesis method. When expressed, this mutagenized nucleic acid encodes a polypeptide that is homologous to the protein encoded by the original RSDRGs. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp (1988) Gene 73:237-244 and Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

In one class of embodiments, the polypeptides herein are at least 70%, generally at least 75%, optionally at least 80%, 85%, 90%, 98% or 99% or more identical to a reference polypeptide, e.g., those that are encoded by DNA sequences as set forth by any one of the RSDRGs disclosed herein or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or more identical to a reference nucleic acid, e.g., those that are set forth by any one of the RSDRGs disclosed herein or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” as applied to nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “polypeptide” is used interchangeably with the terms “polypeptides” and “protein(s)”, and refers to a polymer of amino acid residues. A ‘mature protein’ is a protein which is full-length and which, optionally, includes glycosylation or other modifications typical for the protein in a given cellular environment.

The term “variant” or “mutant” with respect to a polypeptide refers to an amino acid sequence that is altered by one or more amino acids with respect to a reference sequence. The variant may have “conservative” changes, wherein a substituted amino acid has similar structural or chemical properties, e.g., replacement of leucine with isoleucine. Alternatively, a variant may have “nonconservative” changes, e.g., replacement of a glycine with a tryptophan. Analogous minor variation can also include amino acid deletion or insertion, or both. Guidance in determining which amino acid residues can be substituted, inserted, or deleted without eliminating biological or immunological activity can be found using computer programs well known in the art, for example, DNASTAR software.

A variety of additional terms are defined or otherwise characterized herein. In practicing the instrumentalities described herein, many conventional techniques in molecular biology, microbiology, and recombinant DNA are optionally used. These techniques are well known to those of ordinary skill in the art. For example, one skilled in the art would be familiar with techniques for in vitro amplification methods, including the polymerase chain reaction (PCR), for the production of the homologous nucleic acids described herein.

In addition, commercially available kits may facilitate the purification of plasmids or other relevant nucleic acids from cells. See, for example, EasyPrep™ and FlexiPrep™ kits, both from Pharmacia Biotech; StrataClean™ from Stratagene; and, QIAprep™ from Qiagen. Any isolated and/or purified nucleic acid can be further manipulated to produce other nucleic acids, used to transfect cells, incorporated into related vectors to infect organisms, or the like. Typical cloning vectors contain transcription terminators, transcription initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Various types of mutagenesis are optionally used to modify RSDRGs and their encoded polypeptides, as described herein, to produce conservative or non-conservative variants. Any available mutagenesis procedure can be used. Such mutagenesis procedures optionally include selection of mutant nucleic acids and polypeptides for one or more activity of interest. Procedures that can be used include, but are not limited to: site-directed point mutagenesis, random point mutagenesis, in vitro or in vivo homologous recombination (DNA shuffling), mutagenesis using uracil-containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, mutagenesis by chimeric constructs, and many others known to persons of skill in the art.

In one embodiment, mutagenesis can be guided by known information about the naturally occurring molecule or altered or mutated naturally occurring molecule. By way of example, this known information may include sequence, sequence comparisons, physical properties, crystal structure and the like. In another class of mutagenesis, modification is essentially random, e.g., as in classical DNA shuffling.

Polypeptides may include variants, in which the amino acid sequence has at least 70% identity, preferably at least 80% identity, typically 90% identity, preferably at least 95% identity, more preferably at least 98% identity and most preferably at least 99% identity, to the amino acid sequences as encoded by the DNA sequences set forth in any one of the RSDRGs disclosed herein.

The aforementioned polypeptides may be obtained by any of a variety of methods. Smaller peptides (less than 50 amino acids long) are conveniently synthesized by standard chemical techniques and can be chemically or enzymatically ligated to form larger polypeptides. Polypeptides can be purified from biological sources by methods well known in the art, for example, as described in Protein Purification, Principles and Practice, Second Edition Scopes, Springer Verlag, N.Y. (1987) Polypeptides are optionally but preferably produced in their naturally occurring, truncated, or fusion protein forms by recombinant DNA technology using techniques well known in the art. These methods include, for example, in vitro recombinant DNA techniques, synthetic techniques and in vivo genetic recombination. See, for example, the techniques described in Sambrook et al. (2001) Molecular Cloning, A Laboratory Manual, Third Edition, Cold Spring Harbor Press, N.Y.; and Ausubel et al., eds. (1997) Current Protocols in Molecular Biology, Green Publishing Associates, Inc., and John Wiley & Sons, Inc., N.Y (supplemented through 2002). RNA encoding the proteins may also be chemically synthesized. See, for example, the techniques described in Oligonucleotide Synthesis, (1984) Gait ed., IRL Press, Oxford, which is incorporated by reference herein in its entirety.

The nucleic acid molecules described herein may be expressed in a suitable host cell or an organism to produce proteins. Expression may be achieved by placing a nucleotide sequence encoding these proteins into an appropriate expression vector and introducing the expression vector into a suitable host cell, culturing the transformed host cell under conditions suitable for expression of the proteins described or variants thereof, or a polypeptide that comprises one or more domains of such proteins. The recombinant proteins from the host cell may be purified to obtain purified and, preferably, active protein. Alternatively, the expressed protein may be allowed to function in the intact host cell or host organism.

Appropriate expression vectors are known in the art, and may be purchased or applied for use according to the manufacturer's instructions to incorporate suitable genetic modifications. For example, pET-14b, pcDNA1Amp, and pVL1392 are available from Novagen and Invitrogen, and are suitable vectors for expression in E. coli, mammalian cells and insect cells, respectively. These vectors are illustrative of those that are known in the art, and many other vectors can be used for the same purposes. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as Saccharomyces and Aspergillus cells; insect cells such as Drosophila S2 and Spodoptera Sf9 cells, mammalian cells such as CHO, COS, HeLa, 293 cells; and plant cells.

Culturing and growth of the transformed host cells can occur under conditions that are known in the art. The conditions will generally depend upon the host cell and the type of vector used. Suitable culturing conditions may be used such as temperature and chemicals and will depend on the type of promoter utilized.

Purification of the proteins or domains of such proteins, if desired, may be accomplished using known techniques without performing undue experimentation. Generally, the transformed cells expressing one of these proteins are broken, crude purification occurs to remove debris and some contaminating proteins, followed by chromatography to further purify the protein to the desired level of purity. Host cells may be broken by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

In general, RSDRG proteins or domains, or antibodies to such proteins can be purified, either partially (e.g., achieving a 5×, 10×, 100×, 500×, or 1000× or greater purification), or even substantially to homogeneity (e.g., where the protein is the main component of a solution, typically excluding the solvent (e.g., water or DMSO) and buffer components (e.g., salts and stabilizers) that the protein is suspended in, e.g., if the protein is in a liquid phase), according to standard procedures known to and used by those of skill in the art. Accordingly, the polypeptides can be recovered and purified by any of a number of methods well known in the art, including, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against the proteins described herein are used as purification reagents, e.g., for affinity-based purification of proteins comprising one or more RSDRG protein domains or antibodies thereto. Once purified, partially or to homogeneity, as desired, the polypeptides are optionally used e.g., as assay components, therapeutic reagents or as immunogens for antibody production.

In addition to other references noted herein, a variety of purification methods are well known in the art, including, for example, those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana, Bioseparation of Proteins, Academic Press, Inc. (1997); Bollag et al., Protein Methods, 2nd Edition Wiley-Liss, NY; Walker (1996) The Protein Protocols Handbook Humana Press, NJ; Harris and Angal Protein Purification Applications: A Practical Approach IRL Press at Oxford, Oxford, England (1990); Scopes, Protein Purification. Principles and Practice 3rd Edition Springer Verlag, NY (1993); Janson and Ryden, Protein Purification: Principles, High Resolution Methods and Applications, Second Edition Wiley-VCH, NY (1998); and Walker, Protein Protocols on CD-ROM Humana Press, NJ (1998); and the references cited therein.

After synthesis, expression and/or purification, proteins may possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

In another aspect, antibodies to the RSDRG proteins or fragments thereof may be generated using methods that are well known in the art. The antibodies may be utilized for detecting and/or purifying the RSDRG proteins, optionally discriminating the proteins from various homologues. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

General protocols that may be adapted for detecting and measuring the expression of the described RSDRG proteins using the above mentioned antibodies are known. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and other protocols that are commonly used and widely described in scientific and patent literature.

Sequence of the RSDRG genes may also be used in genetic mapping of plants or in plant breeding. Polynucleotides derived from the RSDRG gene sequences may be used in in situ hybridization to determine the chromosomal locus of the RSDRG genes on the chromosomes. These polynucleotides may also be used to detect segregation of different alleles at certain RSDRG loci.

Sequence information of the RSDRG genes may also be used to design oligonucleotides for detecting RSDRG mRNA levels in the cells or in plant tissues. For example, the oligonucleotides can be used in a Northern blot analysis to quantify the levels of RSDRG mRNA. Moreover, full-length or fragment of the RSDRG genes may be used in preparing microarrays (or gene chips). Full-length or fragment of the RSDRG genes may also be used in microarray experiments to study expression profile of the RSDRG genes. High-throughput screening can be conducted to measure expression levels of the RSDRG genes in different cells or tissues. Various compounds or other external factors may be screened for their effects expression of the RSDRG gene expression.

Sequences of the RSDRG genes and proteins may also provide a tool for identification of other proteins that may be involved in plant drought response. For example, chimeric RSDRG proteins can be used as a “bait” to identify other proteins that interact with RSDRG proteins in a yeast two-hybrid screening. Recombinant RSDRG proteins can also be used in pull-down experiment to identify their interacting proteins. These other proteins may be cofactors that enhance the function of the RSDRG proteins, or they may be RSDRG proteins themselves which have not been identified in the experiments disclosed herein.

The RSDRG polypeptides may possess structural features which can be recognized, for example, by using immunological assays. The generation of antisera which specifically bind the RSDRG polypeptides, as well as the polypeptides which are bound by such antisera, are a feature of the disclosed embodiments.

In order to produce antisera for use in an immunoassay, one or more of the immunogenic RSDRG polypeptides or fragments thereof are produced and purified as described herein. For example, recombinant protein may be produced in a host cell such as a bacterial or an insect cell. The resultant proteins can be used to immunize a host organism in combination with a standard adjuvant, such as Freund's adjuvant. Commonly used host organisms include rabbits, mice, rats, donkeys, chickens, goats, horses, etc. An inbred strain of mice may also be used to obtain more reproducible results due to the virtual genetic identity of the mice. The mice are immunized with the immunogenic RSDRG polypeptides in combination with a standard adjuvant, such as Freund's adjuvant, and a standard mouse immunization protocol. See, for example, Harlow and Lane, Antibodies, A Laboratory Manual, Cold Spring Harbor Publications, New York (1988), which provides comprehensive descriptions of antibody generation, immunoassay formats and conditions that can be used to determine specific immunoreactivity. Alternatively, one or more synthetic or recombinant RSDRG polypeptides or fragments thereof derived from the sequences disclosed herein is conjugated to a carrier protein and used as an immunogen.

Antisera that specifically bind the RSDRG proteins may be used in a range of applications, including but not limited to immunofluorescence staining of cells for the expression level and localization of the RSDRG proteins, cytological staining for the expression of RSDRG proteins in tissues, as well as in Western blot analysis.

Another aspect of the disclosure includes screening for potential or candidate modulators of RSDRG protein activity. For example, potential modulators may include small molecules, organic molecules, inorganic molecules, proteins, hormones, transcription factors, or the like, which can be contacted to a cell or certain tissues that express the RSDRG proteins to assess the effects, if any, of the candidate modulator upon RSDRG protein activity.

Alternatively, candidate modulators may be screened to modulate expression of RSDRG proteins. For example, potential modulators may include small molecules, organic molecules, inorganic molecules, proteins, hormones, transcription factors, or the like, which can be contacted to a cell or certain tissues that express the RSDRG proteins, to assess the effects, if any, of the candidate modulator upon RSDRG protein expression. Expression of a RSDRG gene described herein may be detected, for example, via Northern blot analysis or quantitative (optionally real time) RT-PCR, before and after application of potential expression modulators. Alternatively, promoter regions of the various RSDRG genes may be coupled to reporter constructs including, without limitation, CAT, beta-galactosidase, luciferase or any other available reporter, and may similarly be tested for expression activity modulation by the candidate modulator. Promoter regions of the various genes are generally sequences in the proximity upstream of the start site of transcription, typically within 1 Kb or less of the start site, such as within 500 bp, 250 bp or 100 bp of the start site. In certain cases, a promoter region may be located between 1 and 5 Kb from the start site.

In either case, whether the assay is to detect modulated activity or expression, a plurality of assays may be performed in a high-throughput fashion, for example, using automated fluid handling and/or detection systems in serial or parallel fashion. Similarly, candidate modulators can be tested by contacting a potential modulator to an appropriate cell using any of the activity detection methods herein, regardless of whether the activity that is detected is the result of activity modulation, expression modulation or both.

A method of modifying a plant may include introducing into a host plant one or more RSDRG genes described above. The RSDRG genes may be placed in an expression construct, which may be designed such that the RSDRG protein(s) are expressed constitutively, or inducibly. The construct may also be designed such that the RSDRG protein(s) are expressed in certain tissue(s), but not in other tissue(s). The RSDRG protein(s) may enhance the ability of the host plant in drought tolerance, such as by reducing water loss or by other mechanisms that help a plant cope with water deficit growth conditions. The host plant may include any plants whose growth and/or yield may be enhanced by a modified drought response. Methods for generating such transgenic plants is well known in the field. See e.g., Leandro Peñ a (Editor), Transgenic Plants: Methods and Protocols (Methods in Molecular Biology), Humana Press, 2004.

The use of gene inhibition technologies such as antisense RNA or co-suppression or double stranded RNA interference is also within the scope of the present disclosure. In these approaches, the isolated gene sequence is operably linked to a suitable regulatory element. In one embodiment of the disclosure, the construct contains a DNA expression cassette that contains, in addition to the DNA sequences required for transformation and selection in said cells, a DNA sequence that encodes a RSDRG proteins or a RSDRG modulator protein, with at least a portion of said DNA sequence in an antisense orientation relative to the normal presentation to the transcriptional regulatory region, operably linked to a suitable transcriptional regulatory region such that said recombinant DNA construct expresses an antisense RNA or portion thereof of an antisense RNA in the resultant transgenic plant.

It is apparent to one of skill in the art that the polynucleotide encoding the RSDRG proteins or a RSDRG modulator proteins can be in the antisense (for inhibition by antisense RNA) or sense (for inhibition by co-suppression) orientation, relative to the transcriptional regulatory region. Alternatively a combination of sense and antisense RNA expression can be utilized to induce double stranded RNA interference. See, e.g., Chuang and Meyerowitz, PNAS 97: 4985-4990, 2000; see also Smith et al., Nature 407: 319-320, 2000.

These methods for generation of transgenic plants generally entail the use of transformation techniques to introduce the gene or construct encoding the RSDRG proteins or a RSDRG modulator proteins, or a part or a homolog thereof, into plant cells. Transformation of a plant cell can be accomplished by a variety of different methodology. Methods that have general utility include, for example, Agrobacterium based systems, using either binary and/or cointegrate plasmids of both A. tumifaciens and A. rhyzogenies, (See e.g., U.S. Pat. No. 4,940,838, U.S. Pat. No. 5,464,763), the biolistic approach (See e.g, U.S. Pat. No. 4,945,050, U.S. Pat. No. 5,015,580, U.S. Pat. No. 5,149,655), microinjection, (See e.g., U.S. Pat. No. 4,743,548), direct DNA uptake by protoplasts, (See e.g., U.S. Pat. No. 5,231,019, U.S. Pat. No. 5,453,367) or needle-like whiskers (See e.g., U.S. Pat. No. 5,302,523). Any method for the introduction of foreign DNA into a plant cell and for expression therein may be used within the context of the present disclosure.

Plants that are capable of being transformed encompass a wide range of species, including but not limited to soybean, corn, potato, rice, wheat and many other crops, fruit plants, vegetables and tobacco. See generally, Vain, P., Thirty years of plant transformation technology development, Plant Biotechnol J. 2007 March; 5(2):221-9. Any plants that are capable of taking in foreign DNA and transcribing the DNA into RNA and/or further translating the RNA into a protein may be a suitable host.

The modulators described above that may alter the expression levels or the activity of the RSDRG proteins (collectively called RSDRG modulators) may also be introduced into a host plant in the same or similar manner as described above. In one embodiment, the RSDRG modulators are primarily transcription factors that regulate the transcription of the RSDRGs.

The RSDRG proteins or the RSDRG modulators may be used to modify a target plant by causing them to be assimilated by the plant. Alternatively, the RSDRG proteins or the RSDRG modulators may be applied to a target plant by causing them to be in contact with the plant, or with a specific organ or tissue of the plant. In one embodiment, organic or inorganic molecules that can function as RSDRG modulators may be caused to be in contact with a plant such that these chemicals may enhance the drought response of the target plant.

In addition to the RSDRG modulators, RSDRG polypeptides or RSDRG nucleic acids, a composition containing other ingredients may be introduced, administered or delivered to the plant to be modified. In one aspect, a composition containing an agriculturally acceptable ingredient may be used in conjunction with the RSDRG modulators to be administered or delivered to the plant.

Bioinformatic systems are widely used in the art, and can be utilized to identify homology or similarity between different character strings, or can be used to perform other desirable functions such as to control output files, provide the basis for making presentations of information including the sequences and the like. Examples include BLAST, discussed supra. For example, commercially available databases, computers, computer readable media and systems may contain character strings corresponding to the sequence information herein for the RSDRG polypeptides and nucleic acids described herein. These sequences may include specifically the RSDRG sequences listed herein and the various silent substitutions and conservative substitutions thereof.

The bioinformatic systems contain a wide variety of information that includes, for example, a complete sequence listings for the entire genome of an individual organism representing a species. Thus, for example, using the RSDRG sequences as a basis for comparison, the bioinformatic systems may be used to compare different types of homology and similarity of various stringency and length on the basis of reported data. These comparisons are useful to identify homologs or orthologs where, for example, the basic RSDRG gene ortholog is shown to be conserved across different organisms. Thus, the bioinformatic systems may be used to detect or recognize the homologs or orthologs, and to predict the function of recognized homologs or orthologs. By way of example, many homology determination methods have been designed for comparative analysis of sequences of biopolymers including nucleic acids, proteins, etc. With an understanding of hydrogen bonding between the principal bases in natural polynucleotides, models that simulate annealing of complementary homologous polynucleotide strings can also be used as a foundation of sequence alignment or other operations typically performed on the character strings corresponding to the sequences herein. One example of a software package for calculating sequence similarity is BLAST, which can be adapted to the present invention by inputting character strings corresponding to the sequences herein.

The software can also include output elements for controlling nucleic acid synthesis (e.g., based upon a sequence or an alignment of a sequences herein) or other operations which occur downstream from an alignment or other operation performed using a character string corresponding to a sequence herein.

In an additional aspect, kits may embody any of the methods, compositions, systems or apparatus described above. Kits may optionally comprise one or more of the following: (1) a composition, system, or system component as described herein; (2) instructions for practicing the methods described herein, and/or for using the compositions or operating the system or system components herein; (3) a container for holding components or compositions, and, (4) packaging materials.

### EXAMPLES

The following nonlimiting examples report general procedures, reagents and characterization methods that teach by way of example, and should not be construed in a narrowing manner that limits the disclosure to what is specifically disclosed. Those skilled in the art will understand that numerous modifications may be made and still the result will fall within the spirit and scope of the present invention.

### Example 1

### Differential Gene Expression of Drought Response Genes in Soybean Roots

The overall goal of these experiments is to identify potential gene(s) that may confer upon a plant the capability to grow and reproduce under water deficit conditions. The first step towards accomplishing this goal is to characterize the transcription profiles in regions of growth maintenance and inhibition in soybean roots under water deficit conditions in comparison to well-watered conditions. Another approach taken in this disclosure is to compare the transcription profiles in the root regions of soybean with those from other plants, such as maize. One specific objective is to characterize the transcriptome of apical and basal regions of soybean root growth zone under water deficit conditions for a short (5 h) and long (48 h) period. Soybean Genechips manufactured by Affymetrix were used.

Briefly, young Magellan soybean plants were subject to water deficit stress treatment for 5 h or 48 h. Root regions 1 and 3 from well watered samples and root regions 1 and 2 from water stressed (or deficit) samples were collected in triplicate. Total RNA isolation and microarray hybridizations were conducted using standard protocols such as the protocols described in U.S. patent application Ser. No. 12/138,392 (US2009/0210968). 60K soybean Affymetrix GeneChip (total 30) and the pair wise comparison experimental plan were used for the microarray experiments.

More specifically, a soybean seedling system was used for these experiments. Soybean (Glycine max L.) line Magellan seeds were sterilized with 20% bleach for 2 minutes and rinsed with running tap water for about 20 minutes. Seeds were then placed on germination paper saturated with 10 mM CaCl₂ and 10 mM Ca(NO₃)₂ solutions and germinated in the dark at 29° C. and nearly 100% relative humidity. A mixture of vermiculite and Turface at a 1:1 volumetric ratio was used as the culture medium. Plastic tubes covered with fiberglass mesh at the bottom and plastic boxes were used as growth containers and were filled with either well-watered (pre-soaked with 10 mM CaCl₂ and 10 mM Ca(NO₃)₂ solution) or water-stressed medium. The water-stressed medium was pre-mixed with 10 mM CaCl₂ and 10 mM Ca(NO₃)₂ solutions to a water potential of about −1.6 MPa as measured with isopiestic thermocouple psychrometers (Boyer and Knipling, 1965). The plastic tubes and boxes containing culture medium were then placed in the humid room, where the parameters were set for seedling growth (temperature: 29° C. and 100% RH, no light), for one day before transplanting.

Uniform seedlings with a root length of 11-25 mm were transplanted to boxes and tubes containing either well-watered or water-stressed medium. At 5 h, 12 h, 24 h, and 48 hours after transplanting, three biological replicates of soybean seedlings were harvested. Samples from 5 h and 48 h timpoints (earlier and later stages of water deficit) after transplanting were used for the microarray analysis. Samples from 5 h, 12 h, 24 h, and 48 hours after transplanting were used to conduct qRTPCR analysis of the transcription factors and other selected genes from the microarray results. The 5 h, 12 h, and 24 h time points were chosen to permit the identification of early-responsive transcription factors after the initiation of water-stress treatment and before root growth reached the steady state (Sharp et al. 2004). The 48 h time point was selected to identify transcription factors that might be responsible for the changes associated with the steady-state patterns of the relative elongation rate of soybean seedling roots in response to water-stress conditions.

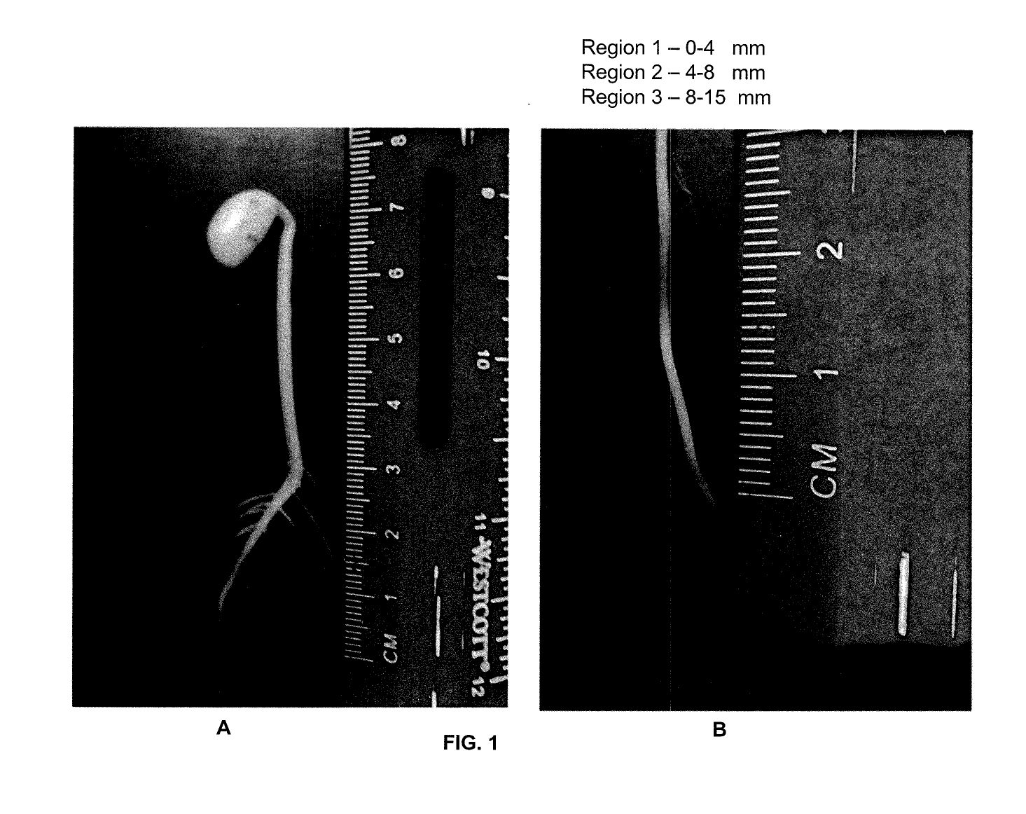

The soybean seedlings were grown to about 6-10 cm tall. The primary roots of these soybeans seedlings were dissected into 3 regions based on previously characterized expansion profiles: Region 1(R1) is 0-4 mm from the root tip (including the root cap), Region 2 (R2) is 4-8 mm from the root tip, and Region 3 (R3) is 8-15 mm from the root tip. See FIG. 1. Regions 1-2 and regions 1-3 were collected in water-stressed roots and well-watered roots, respectively. These root regions were immediately frozen in liquid nitrogen and then stored at −80° C. for RNA isolation and microarray analysis. Comparison of gene expression between region 2 of water-stressed roots and region 3 of well-watered roots may help distinguish the responses induced by water stress from responses resulting from changes in normal growth deceleration.

The soybean primary root adapts to low water potential (e.g., Ψ_{w}, −1.6 MPa) by maintaining longitudinal expansion in the apical 4 mm (region 1), whereas in the adjacent 4 mm (region 2) longitudinal expansion reaches a maximum in well-watered roots but is progressively inhibited at low Ψ_{w} (Yamaguchi et al. 2009). To identify mechanisms that determine these responses to low Ψ_{w}, and to elucidate the regulatory networks involved, the gene expression patterns in these regions of water-stressed and well-watered roots were profiled. Also, the gene expression between region 2 of water-stressed roots and the growth deceleration zone in well-watered roots (region 3) were compared to help distinguish stress-responsive genes in region 2 from those involved in cell maturation. The results reveal that stress-responsive transcripts are largely root region specific. The differentially expressed transcripts (also termed “DET”) were then compared to the available soybean genome sequence information, and the major metabolic and transcriptional regulatory pathways involved in the response to water deficit conditions.

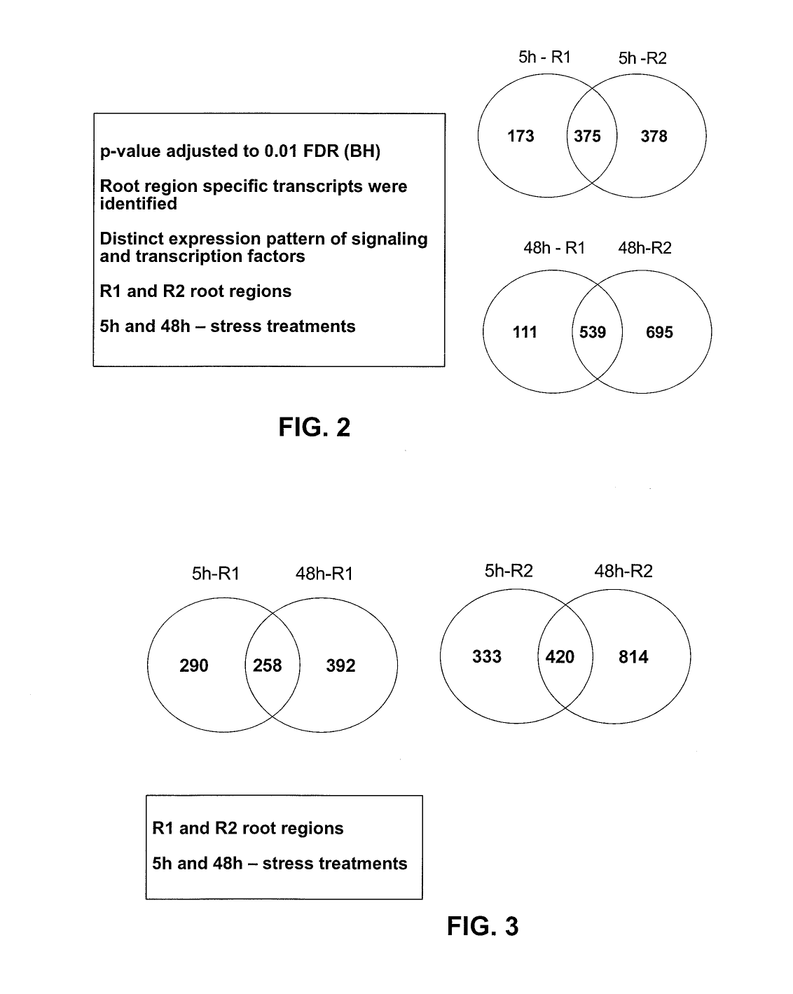

FIG. 2 shows the numbers of genes that showed differential expression at the same time point between different root regions. As shown in the upper panel of FIG. 2, 548 (173+375) genes show differential expression in the R1 region after 5 hour under drought conditions (Table 1). By contrast, 753 (378+375) genes show differential expression in the R2 region after 5 hour under drought conditions (Table 2). Among these genes, 375 genes are differentially expressed after 5 hour under water deficit conditions in both the R1 and R2 regions (Table 3), while 173 genes show R1 specific differential expression and 378 genes show R2 specific differential expression at the 5 hour time point.

As shown in the lower panel of FIG. 2, 650 (111+539) genes show differential expression in the R1 region after 48 hour under drought conditions (Table 4). By contrast, 1234 (695+539) genes show differential expression in the R2 region after 48 hour under drought conditions (Table 5). Among these genes, 539 genes are differentially expressed after 48 hour under water deficit conditions in both the R1 and R2 regions (Table 6), while 111 genes show R1 specific differential expression and 695 genes show R2 specific differential expression at the 48 hour time point.

The number of genes that are differentially expressed within the same root region are compared between the two time points, namely, 5 hr and 48 hr after the plants have been subjected to water deficit conditions (FIG. 3). Out of the 548 genes found to be differentially expressed in the R1 region after 5 hour under water deficit conditions (Table 7), 290 genes are differentially expressed in the R1 region only after 5 hours, but not after 48 hours, under drought conditions (Table 8). Out of the 650 genes found to be differentially expressed after 48 hour under water deficit conditions (Table 9), 392 genes are differentially expressed in the R1 region only after 48 hours, but not after 5 hours, under drought conditions (Table 10). Similarly, out of the 753 genes found to be differentially expressed in the R2 region after 5 hour under water deficit conditions (Table 11), 333 genes are differentially expressed in the R1 region only after 5 hours, but not after 48 hours, under drought conditions (Table 12). Out of the 1234 genes found to be differentially expressed in the R2 region after 48 hour under water deficit conditions (Table 13), 814 genes are differentially expressed in the R2 region only after 48 hours, but not after 5 hours, under drought conditions (Table 14).

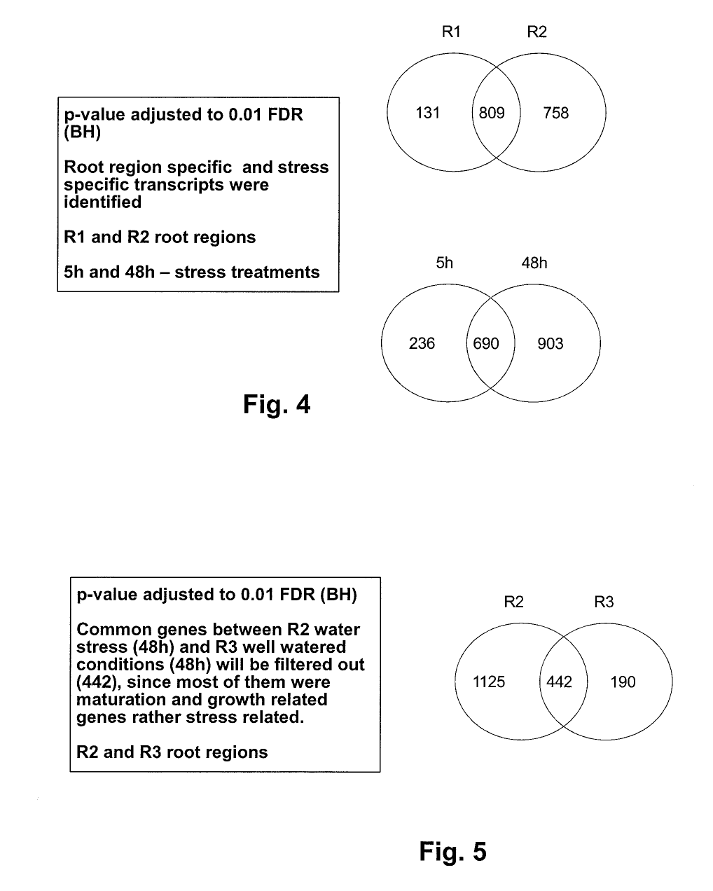

The upper panel of FIG. 4 shows genes that are only differentially expressed in R1, R2 or both R1 and R2 regions of the root up to 48 hours after the plants are exposed to drought conditions, which include the 5 h and 48 h time points. Out of a total of 940 (290+258+392 as shown in FIG. 3) genes that are differentially expressed at 5 hr and 48 hour after being exposed to drought conditions, 131 genes are only differentially expressed in the R1 region (Table 15). Out of a total of 1567 (333+420+814 as shown in FIG. 3) genes that are differentially expressed at 5 hr and 48 hour after being exposed to drought conditions, 758 genes show differential expression under drought conditions only in R2 region of the root (Table 16). Interestingly, 809 genes show differential expression in both R1 and R2 regions after 5 hr or 48 hour exposure to drought conditions.

As shown in the lower panel of FIG. 4, out of the total of 926 genes that show differential expression in both R1 and R2 regions 5 hours after being subjected to drought conditions, 236 genes show differential expression after 5 hour, but not after 48 hours exposure to drought conditions. Differential expression was observed in 903 genes only after 48 hours, but not at 5 hours after exposure to drought conditions. 690 genes show differential expression both at 5 hours and 48 hours after exposure to drought conditions.

As shown in FIG. 5, 1125 genes were found to be differentially expressed under drought condition only in the R2 region but not in the R3 region. Conversely, 190 genes are differentially expressed under drought condition only in the R3 region but not in the R2 region. The R2 and R3 regions also share 442 genes that are commonly expressed in both regions under drought conditions. Most of these 442 genes likely represent genes that are implicated in maturation and/or growth of the roots, but are not specifically induced by the water deficit conditions.

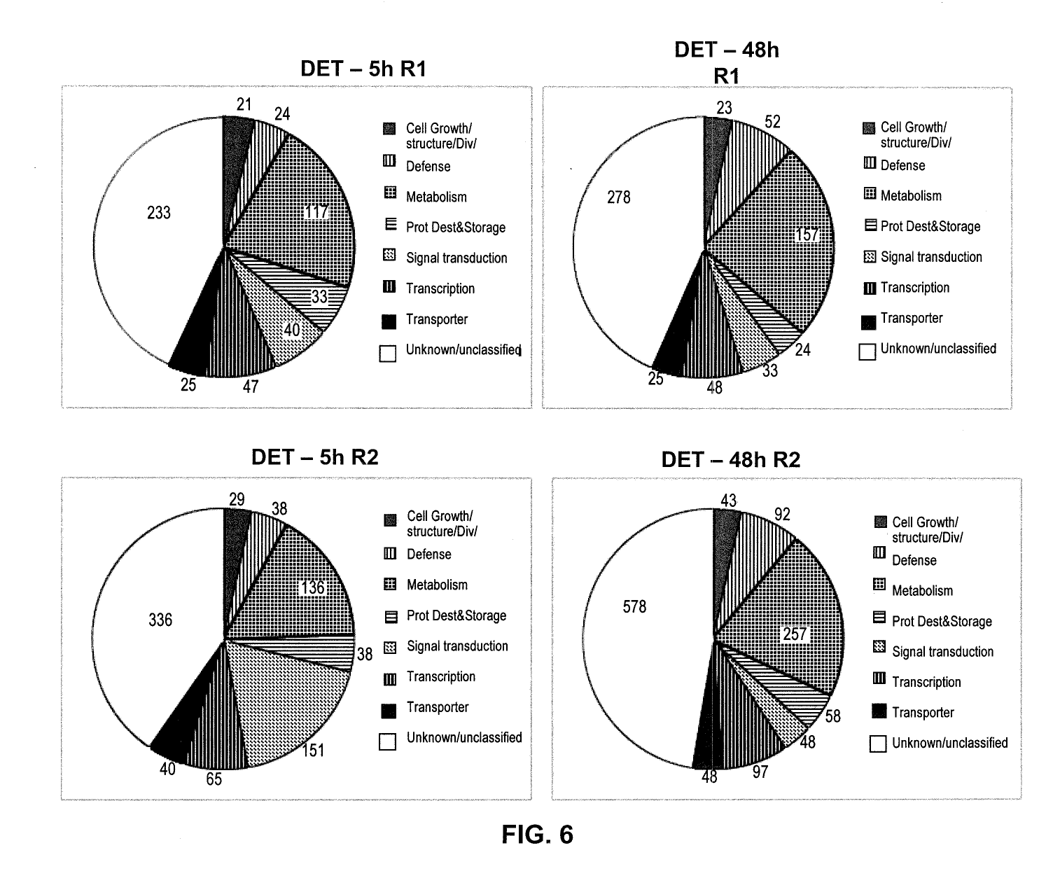

FIG. 6 presents pie charts illustrating the different functional categories of genes that show differential expression in the R1 (8A and 8B) or R2 (8C and 8D) regions after having been subjected to water deficit conditions for 5 hour (8A and 8C) or 48 hours (8B and 8D). DET stands for “Differentially Expressed Transcripts.”

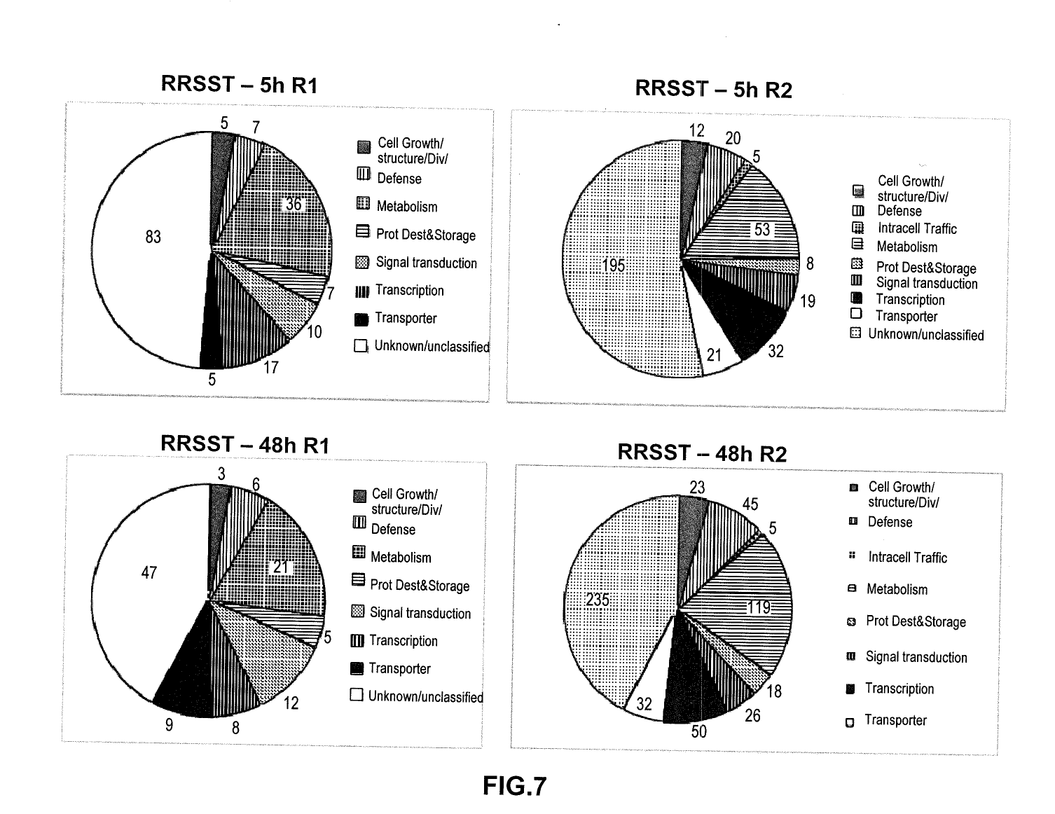

FIG. 7 shows the different categories of the “root region and stress specific transcripts” (RRSST) by their known or predicted function.

### Example 2

### Comparison of the Gene Expression Patterns Under Drought Conditions in the Root Regions of Soybean and Maize

To gain insight into whether the gene expression pattern in response to drought differs among different species, the root region specific and stress specific gene expression patterns of monocot (Maize) and dicot (Soybean) were compared. These analyses confirmed that a species-specific gene expression pattern exists under water deficit conditions in root tissues.

Microarray studies were conducted in maize using the same seedling root experimental conditions as used in the soybean system described in Example 1. Based on the maize array results (root region specific and stress specific genes) and the results from other experiments and also from the reported gene information in the literature, a panel/subset of 273 genes were selected for further testing. In order to further characterize the gene expression under various time points of water deficit conditions in maize roots, high throughput qRT-PCR was utilized for more precise and sensitive data points. These 273 genes (listed in Table 17) were found to be differentially expressed in various root regions of maize at different stress time points (0, 3, 6, 12, 24, 48 hours of stress). The actual sequences of 224 genes out of these 273 obtained from the maize genome database are disclosed herein as SEQ ID. Nos. 1-224. The remaining 49 genes all have GenBank accession numbers whose sequences can be obtained for free from the GenBank database maintained by NCBI.

Because the root system of soybean and maize are similar, it is likely that soybean homologs of these genes also play some roles in stress avoidance mechanism by maintaining root growth under water deficit conditions. Soybean homologs of these 273 maize genes are identified by Blast searching and the sequences of some of these soybean homologs are disclosed herein as SEQ ID. Nos. 225-432.

A number of genes were found to show differential expression in root region R1 5 hours after being subjected to drought conditions in both soybean and maize. Examples of such genes include root specific protein RCc3, ionositol oxygenase, Lipoxygenase, among others. A number of such genes are listed in Table 18 showing sequence comparison between the drought response genes from soybean and their counterparts from maize.

At least 30 common genes show differential expression in root region R25 hours after being subjected to drought conditions in both soybean and maize. Examples of such genes include bZip, DREB, ACC oxi, CDPK, etc. Table 19 lists some of these genes from both soybean and maize and the sequence similarity between the soybean genes and their respective maize counterparts.

At least 18 genes show differential expression in root region R1 after 48 hours under drought conditions in both soybean and maize. Examples of such genes include put. NAC, CCAAT, put. Myb, GST, etc. Table 20 lists some of these genes from both soybean and maize and the sequence similarity between the soybean genes and their respective maize counterparts.

At least 29 genes show differential expression in root region R2 after 48 hour under drought conditions in both soybean and maize. Examples of such genes include NAC, a-Galact, CCAAT, SAPK3, HSP20, etc. Table 21 lists some of these genes from both soybean and maize and the sequence similarity between the soybean genes and their respective maize counterparts.

As described above, microarray experiments were performed in maize and soybean seedling root system with substantially the same water deficit conditions and similar root segment size. Soybean genes differentially expressed under water deficit conditions were compared with the maize microarray results. Those soybean genes common between soybean and maize whose expression was found to be altered under water deficit conditions were identified. The sequences of these soybean genes are disclosed herein as SEQ ID. Nos. 433-480. Because these genes have homologs in both soybean and maize whose expression levels are altered in response to drought conditions, these genes are likely to encode proteins that play a role in drought response in a plant.

### Example 3

### Confirmation of Root Drought Response Genes and Creation of Transgenic Plants

The genes shown to be up-regulated or down-regulated were confirmed by comparing the expression levels of specific genes in the root regions under drought or normal conditions by using Northern blot or quantitative RT-PCR. Genes that have been confirmed were cloned and placed in different expression constructs so that they could be introduced into different host plant.

The genes found to be upregulated or downregulated in the root regions of soybean and/or maize may be altered in a host plant such as soybean or maize in order to generate genetically altered plants that are more resistant to water deficit conditions. Alternatively, one or more of these genes may be introduced into a host plant and forced to be expressed at an elevated level or at a specific location in order to create a genetically altered plant that is more tolerant to water deficit conditions.

Table 22 shows the names of the genes that have been confirmed and selected to be introduced into a host plant, such as Arabidopsis, soybean and maize in order to obtain transgenic plants that are more tolerant to drought conditions than the non-transgenic host plant. The sequences of most of the genes listed in Table 22 are also disclosed as SEQ ID Nos. 481-510. Other sequences can be obtained from the GenBank database or various soybean, maize and arabidopsis databases. The promoters used for most of the constructs are also disclosed in Table 22.

While the foregoing instrumentalities have been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

### REFERENCES

In addition to those references that are cited in full in the text, additional information for those abbreviated citations is listed below. The content of all patents, patent applications or other publications cited in this disclosure are incorporated by reference into this disclosure.
Boyer, J S, 1983, Environmental stress and crop yields. In C. D. Raper and P. J. Kramer (ed) Crop reactions to water and temperature stresses In humid, temperature climates. Westview press, Boulder, Colo. pp 3-7.
Muchow R C, Sinclair T R. 1988. Water and nitrogen limitations In soybean grain production. II. Field and model analyses. Field Crop Res. 15:143-158.
Specht J E, Hume D J, Kumind S V. 1999. Soybean yield potential-A genetic physiological perspective. Crop Science 39:1560-1570.
Neumann P M: Coping mechanisms for crop plants in drought-prone environments. Ann Bot 2008, 101:901-907.
Wang W, Vinocur B, Altman A: Plant responses to drought, salinity and extreme temperatures: towards genetic engineering for stress tolerance. Planta 2003, 218:1-14.
Vinocur, B, Altman A: Recent advances in engineering plant tolerance to abiotic stress: achievements and limitations. Curr Opin Biotech 2005, 16:123-32.
Chaves M M, Oliveire M M: Mechanisms underlying plant resilience to water deficits: prospects for water-saving agriculture. J Exp Bot 2004, 55; 2365-2384.
Shinozaki K, Yamaguchi-Shinozaki K, Seki M: Regulatory network of gene expression in the drought and cold stress responses. Curr Opin Plant Biol 2003, 6:410-417.
Schena M, Shalon D, Davis R W, Brown P O (1995) Quantitative monitoring of gene expression patterns with a complementary DNA microarray. Science 270: 467-470
Shalon D, Smith S, Brown P (1990) A DNA microarray system for analyzing complex DNA samples using two-color fluorescent probe hybridization. Genome Res. 8: 639-645.
Bray E A: Genes commonly regulated by water-deficit stress in Arabidopsis thaliana. J Exp Bot 2004, 55:2331-2341.
Denby K, Gehring C: Engineering drought and salinity tolerance in plants: lessons from genome-wide expression profiling In Arabidopsis. Trends in Plant Sci 2005, 23547-552.
Shinozaki K, Yamaguchi-Shinozaki K: Molecular responses to drought and cold stress. Curr Opin Biotech 1996, 7:181-167
Shinozaki. K. and Yamaguchi-Shinozaki, K: Molecular responses to dehydration and low temperature; differences and cross-talk between two stress signaling pathways. Curr Opin Plant Biol 2000, 3:217-223.
Seki M, Narusaka M, Abe H, Kasuga M, Yamaguchi-Shinozaki K, Carninci P, Hayashizaki Y, Shinozaki K: Monitoring the expression pattern of 1300 Arabidopsis genes under drought and cold stresses by using a full-length cDNA microarray. Plant Cell 2001, 13:61-72.
Fowler S, Thomashow M F: Arabidopsis transcriptome profiling indicates that multiple regulatory pathways are activated during cold acclimation In addition to the CBF cold response pathway, Plant Cell 2002, 14:1875-1690.
Maruyama K, Sakuma Y, Kasuga M, Ito Y, Seki M, Goda H, Shimada Y, Yoshida S, Shinozaki K, Yamaguchi-Shinozaki K: Identification of cold-inducible downstream genes of the Arabidopsis DREB1A/CBF3 transcriptional factor using two microarray systems. Plant J 2004, 38:982-993.
Yamaguchi M, Valliyodan B, Zhang J, LeNoble M E, Yu O, Nguyen H T, Sharp R E Proteomic analysis of the differential responses of elongation rate to water stress within the soybean primary root growth zone. Plant, Cell & Environment 2009. [Epub ahead of print]

TABLE 1

| | | Fold | Soybean_Gen | |
|---|---|---|---|---|
| ID | Gene Name | Change | Bank_Acc | Functional Category |
| 1 | Gma.4058.1.S1_at | 29.77 | AW310338 | Transporter |
| 2 | GmaAffx.19116.1.S1_at | 18.56 | BI968419 | Signal Transduction |
| 3 | Gma.10921.1.S1_s_at | 18.02 | BQ611250 | No Homology to Known |
| | | | | Proteins |
| 4 | Gma.4071.1.S1_at | 12.74 | BG651692 | Unclassified - Hypothetical |
| | | | | Protein Supported by cDNA |
| 5 | GmaAffx.32345.1.A1_s_at | 12.69 | BI970277 | Cell Structure |
| 6 | GmaAffx.89075.1.S1_s_at | 10.01 | CK606454 | No Homology to Known |
| | | | | Proteins |
| 7 | GmaAffx.93260.1.S1_at | 9.745 | CF808985 | Signal Transduction |
| 8 | Gma.5689.3.S1_s_at | 8.693 | BE657645 | Protein Destination & Storage |
| 9 | GmaAffx.50677.1.S1_s_at | 6.936 | BI971744 | No Homology to Known |
| | | | | Proteins |
| 10 | Gma.152.1.S1_at | 6.928 | Y10492.1 | Metabolism |
| 11 | Gma.8416.1.S1_at | 6.715 | U13987.1 | Metabolism |
| 11 | Gma.8416.1.S1_at | 6.715 | U13987.1 | Metabolism |
| 11 | Gma.8416.1.S1_at | 6.715 | U13987.1 | Metabolism |
| 12 | GmaAffx.9866.1.S1_at | 6.693 | BM524432 | Metabolism |
| 13 | Gma.17789.1.S1_at | 6.487 | BG511704 | Unclassified - Hypothetical |
| | | | | Protein Supported by cDNA |
| 14 | Gma.5629.1.S1_at | 6.247 | BQ740550 | Disease & Defense |
| 5 | Gma.320.1.S1_at | 5.971 | AF019116.1 | Disease & Defense |
| 16 | Gma.3804.1.A1_at | 5.568 | AW310249 | Disease & Defense |
| 17 | GmaAffx.11276.1.S1_s_at | 5.327 | AW567677 | Unclassified - Hypothetical |
| | | | | Protein Supported by cDNA |
| 18 | GmaAffx.73185.1.S1_at | 5.042 | BI701903 | No Homology to Known |
| | | | | Proteins |
| 19 | GmaAffx.90960.1.A1_s_at | 4.941 | CF806685 | Signal Transduction |
| 20 | GmaAffx.87679.1.S1_at | 4.865 | BM523734 | Metabolism |
| 21 | Gma.15523.1.S1_s_at | 4.791 | BE475550 | Cell Structure |
| 22 | Gma.17961.1.S1_at | 4.65 | AW350363 | Protein Destination & Storage |
| 23 | GmaAffx.10340.1.S1_at | 4.508 | CA938421 | No Homology to Known |
| | | | | Proteins |
| 24 | Gma.1248.1.S1_s_at | 4.501 | AJ518837.1 | Metabolism |
| 25 | GmaAffx.93073.1.S1_s_at | 4.465 | CF808798 | Cell Structure |
| 26 | Gma.16832.1.S1_s_at | 4.411 | L22029.1 | No Homology to Known |
| | | | | Proteins |
| 27 | Gma.5816.1.S1_at | 4.336 | BI787790 | Unclassified - Hypothetical |
| | | | | Protein Supported by cDNA |
| 28 | Gma.3888.2.S1_at | 4.335 | BM139532 | Unclassified - Hypothetical |
| | | | | Protein NOT Supported by |
| | | | | cDNA |
| 29 | GmaAffx.87679.2.S1_s_at | 4.332 | AW102083 | Metabolism |
| 30 | GmaAffx.18566.1.A1_at | 4.291 | BU550417 | Unclassified - Hypothetical |
| | | | | Protein NOT Supported by |
| | | | | cDNA |
| 31 | GmaAffx.93541.2.S1_at | 4.208 | CF805889 | Protein Destination & Storage |
| 32 | Gma.1221.1.S1_s_at | 4.204 | L23854.1 | Metabolism |
| 32 | Gma.1221.1.S1_s_at | 4.204 | L23854.1 | Metabolism |
| 32 | Gma.1221.1.S1_s_at | 4.204 | L23854.1 | Metabolism |
| 33 | Gma.16425.3.S1_s_at | 4.126 | AW508123 | Unclassified - Protein with |
| | | | | Unknown Function |
| 34 | Gma.16425.2.S1_s_at | 4.078 | AW099501 | Unclassified - Hypothetical |
| | | | | Protein NOT Supported by |
| | | | | cDNA |
| 35 | Gma.16308.1.S1_s_at | 4.027 | U64916.1 | Unclassified - Hypothetical |
| | | | | Protein NOT Supported by |
| | | | | cDNA |
| 36 | GmaAffx.93529.1.S1_s_at | 3.791 | CF809254 | No Homology to Known |
| | | | | Proteins |
| 37 | GmaAffx.10340.1.S1_x_at | 3.77 | CA938421 | No Homology to Known |
| | | | | Proteins |
| 38 | GmaAffx.83638.1.S1_at | 3.768 | BU549896 | Unclassified - Hypothetical |
| | | | | Protein NOT Supported by |
| | | | | cDNA |
| 39 | GmaAffx.9605.1.S1_at | 3.734 | BM091603 | No Homology to Known |
| | | | | Proteins |
| 40 | GmaAffx.70875.1.S1_s_at | 3.656 | AW100693 | No Homology to Known |
| | | | | Proteins |
| 41 | Gma.3405.1.S1_s_at | 3.606 | AW350922 | Metabolism |
| 42 | Gma.6555.1.S1_at | 3.568 | BM954521 | Unclassified - Hypothetical |
| | | | | Protein Supported by cDNA |
| 43 | Gma.4701.2.A1_a_at | 3.545 | BM526048 | Energy |
| 44 | Gma.1400.2.S1_at | 3.516 | BG510739 | Signal Transduction |
| 45 | GmaAffx.37477.1.S1_s_at | 3.499 | BQ081355 | Metabolism |
| 46 | GmaAffx.67477.2.S1_at | 3.453 | AW509184 | Cell Structure |
| 47 | GmaAffx.91346.1.S1_x_at | 3.41 | CF807071 | Unclassified - Hypothetical |
| | | | | Protein Supported by cDNA |
| 48 | GmaAffx.11858.1.A1_s_at | 3.402 | AW351023 | Metabolism |
| 49 | GmaAffx.92537.1.S1_at | 3.401 | CF808262 | Energy |
| 50 | Gma.3639.1.S1_at | 3.4 | AB083030.1 | Protein Destination & Storage |
| 51 | Gma.6288.1.S1_at | 3.388 | AW348556 | Transporter |
| 52 | Gma.5483.1.S1_s_at | 3.362 | CD414581 | Transcription |
| 53 | GmaAffx.85409.1.S1_at | 3.337 | BF598665 | Unclassified - Hypothetical |
| | | | | Protein NOT Supported by |
| | | | | cDNA |
| 54 | Gma.17559.1.S1_at | 3.31 | BG508388 | No Homology to Known |
| | | | | Proteins |
| 55 | GmaAffx.87812.2.S1_at | 3.31 | BI971676 | Signal Transduction |
| 56 | GmaAffx.87526.1.S1_at | 3.286 | BM954581 | Unclassified - Hypothetical |
| | | | | Protein Supported by cDNA |
| 57 | Gma.13447.1.A1_at | 3.285 | CD398305 | No Homology to Known |
| | | | | Proteins |
| 58 | GmaAffx.93462.1.S1_s_at | 3.272 | CF809187 | Signal Transduction |
| 59 | GmaAffx.24352.1.S1_at | 3.243 | BU544287 | No Homology to Known |
| | | | | Proteins |
| 60 | Gma.7332.1.S1_at | 3.196 | BI945636 | Unclassified - Hypothetical |
| | | | | Protein Supported by cDNA |
| 61 | GmaAffx.81930.1.S1_at | 3.194 | BG789998 | Unclassified - Hypothetical |
| | | | | Protein Supported by cDNA |
| 62 | Gma.8896.1.S1_at | 3.179 | BQ252812 | Transcription |
| 63 | Gma.14471.2.S1_at | 3.168 | CD408292 | Transporter |
| 64 | Gma.3149.1.S1_a_at | 3.154 | BU544575 | No Homology to Known |
| | | | | Proteins |
| 65 | GmaAffx.68088.1.S1_at | 3.1 | BG507880 | Signal Transduction |
| 66 | GmaAffx.3905.1.S1_s_at | 3.085 | CA818952 | Unclassified - Protein with |
| | | | | Unknown Function |
| 67 | GmaAffx.54265.1.S1_s_at | 3.068 | BU546096 | Signal Transduction |
| 68 | Gma.4226.2.S1_at | 3.037 | BM568170 | Unclassified - Hypothetical |
| | | | | Protein Supported by cDNA |
| 69 | Gma.8301.2.S1_at | 3.014 | BU544524 | Signal Transduction |
| 70 | GmaAffx.62542.1.S1_at | 2.983 | BE659136 | Unclassified - Hypothetical |
| | | | | Protein NOT Supported by |
| | | | | cDNA |
| 71 | GmaAffx.3905.3.S1_at | 2.969 | BG047142 | Unclassified - Protein with |
| | | | | Unknown Function |
| 72 | GmaAffx.31180.1.A1_at | 2.939 | BE820254 | No Homology to Known |
| | | | | Proteins |
| 73 | GmaAffx.85264.1.S1_at | 2.932 | BE058913 | Metabolism |
| 74 | GmaAffx.90947.1.S1_at | 2.927 | CF806672 | Unclassified - Protein with |
| | | | | Unknown Function |
| 75 | GmaAffx.2743.1.S1_s_at | 2.913 | BM568087 | Signal Transduction |
| 76 | Gma.43.1.A1_at | 2.884 | L12258.1 | Transporter |
| 77 | GmaAffx.40860.2.S1_at | 2.85 | BG509282 | Disease & Defense |
| 78 | Gma.2548.1.S1_at | 2.841 | BE821472 | Unclassified - Hypothetical |
| | | | | Protein Supported by cDNA |
| 79 | GmaAffx.43291.1.S1_at | 2.782 | AI442332 | Metabolism |
| 80 | Gma.4526.1.S1_at | 2.781 | BI967711 | Transcription |
| 81 | GmaAffx.10773.1.S1_at | 2.769 | BU926045 | Unclassified - Hypothetical |
| | | | | Protein Supported by cDNA |
| 82 | GmaAffx.85374.1.S1_at | 2.76 | BI317002 | Signal Transduction |
| 83 | Gma.11259.1.S1_at | 2.733 | AI442316 | Unclassified - Hypothetical |
| | | | | Protein Supported by cDNA |
| 84 | GmaAffx.4905.1.S1_at | 2.73 | BQ453808 | Metabolism |
| 85 | Gma.10756.1.S1_s_at | 2.682 | BE822092 | Unclassified - Hypothetical |
| | | | | Protein Supported by cDNA |
| 86 | Gma.4051.1.S1_at | 2.682 | L22162.1 | Cell Structure |
| 87 | Gma.7474.1.A1_at | 2.672 | BU547968 | Unclassified - Hypothetical |
| | | | | Protein Supported by cDNA |
| 88 | Gma.17605.1.S1_at | 2.637 | BI968208 | Secondary Metabolism |
| 89 | Gma.6266.1.A1_at | 2.593 | CD413418 | Unclassified - Protein with |
| | | | | Unknown Function |
| 90 | Gma.340.1.S1_at | 2.59 | AW350127 | Cell Structure |
| 91 | Gma.13361.1.S1_at | 2.588 | AW309932 | Unclassified - Protein with |
| | | | | Unknown Function |
| 92 | GmaAffx.16502.1.A1_at | 2.585 | BE659118 | No Homology to Known |
| | | | | Proteins |
| 93 | Gma.4336.1.S1_at | 2.564 | BQ079907 | Unclassified - Hypothetical |
| | | | | Protein NOT Supported by |
| | | | | cDNA |
| 94 | GmaAffx.81783.1.S1_at | 2.559 | AW101961 | Signal Transduction |
| 95 | GmaAffx.8098.1.S1_at | 2.546 | AW156410 | No Homology to Known |
| | | | | Proteins |
| 96 | Gma.184.1.S1_at | 2.534 | AW101261 | Unclassified - Hypothetical |
| | | | | Protein Supported by cDNA |
| 97 | GmaAffx.9400.1.S1_at | 2.528 | BI893703 | Signal Transduction |
| 98 | Gma.3522.1.S1_at | 2.526 | BM140209 | Unclassified - Hypothetical |
| | | | | Protein Supported by cDNA |
| 99 | GmaAffx.40453.1.S1_at | 2.501 | BE658683 | No Homology to Known |
| | | | | Proteins |
| 100 | GmaAffx.18518.1.S1_at | 2.498 | BI700329 | Cell Structure |
| 101 | Gma.12356.1.A1_at | 2.497 | CD397798 | Unclassified - Hypothetical |
| | | | | Protein NOT Supported by |
| | | | | cDNA |
| 102 | Gma.9141.1.A1_at | 2.484 | AW310721 | Secondary Metabolism |
| 103 | GmaAffx.87812.1.S1_at | 2.455 | AW831390 | Signal Transduction |
| 104 | GmaAffx.87547.2.S1_at | 2.45 | BQ080473 | Cell Structure |
| 105 | GmaAffx.91346.1.S1_at | 2.437 | CF807071 | Unclassified - Hypothetical |
| | | | | Protein Supported by cDNA |
| 106 | GmaAffx.65829.1.A1_at | 2.428 | CD392418 | Transcription |
| 107 | GmaAffx.78636.1.S1_at | 2.428 | BQ627652 | No Homology to Known |
| | | | | Proteins |
| 108 | GmaAffx.5264.1.S1_s_at | 2.415 | BM521512 | Disease & Defense |
| 109 | GmaAffx.89512.1.A1_at | 2.403 | CK606530 | No Homology to Known |
| | | | | Proteins |
| 110 | Gma.368.1.S1_at | 2.387 | AW349594 | No Homology to Known |
| | | | | Proteins |
| 111 | GmaAffx.61907.1.S1_at | 2.376 | CA819279 | No Homology to Known |
| | | | | Proteins |
| 112 | GmaAffx.83598.1.S1_at | 2.373 | BU964918 | Transcription |
| 113 | Gma.4396.1.S1_at | 2.368 | BQ081294 | No Homology to Known |
| | | | | Proteins |
| 114 | Gma.17887.1.S1_s_at | 2.367 | BG042115 | Unclassified - Hypothetical |
| | | | | Protein Supported by cDNA |
| 115 | Gma.1521.1.S1_at | 2.363 | CD394504 | Cell Structure |
| 116 | Gma.6343.1.S1_at | 2.356 | AI442963 | Unclassified - Hypothetical |
| | | | | Protein NOT Supported by |
| | | | | cDNA |
| 117 | GmaAffx.55404.1.S1_at | 2.351 | BM271530 | No Homology to Known |
| | | | | Proteins |
| 118 | GmaAffx.50347.1.S1_at | 2.342 | BQ740576 | Signal Transduction |
| 119 | GmaAffx.21211.1.S1_at | 2.335 | BU550606 | Protein Destination & Storage |
| 120 | Gma.2488.1.S1_at | 2.327 | CD406079 | Protein Destination & Storage |
| 121 | Gma.3104.1.A1_at | 2.314 | BG839320 | Signal Transduction |
| 122 | GmaAffx.12370.1.S1_at | 2.312 | BE059411 | Cell Growth & Division |
| 123 | Gma.17300.1.A1_at | 2.289 | AW350946 | Unclassified - Hypothetical |
| | | | | Protein NOT Supported by |
| | | | | cDNA |
| 124 | Gma.16913.1.S1_s_at | 2.287 | BI426712 | Unclassified - Protein with |
| | | | | Unknown Function |
| 125 | GmaAffx.33259.1.S1_at | 2.272 | BQ785322 | Unclassified - Hypothetical |
| | | | | Protein Supported by cDNA |
| 126 | GmaAffx.85255.1.S1_at | 2.265 | BE059755 | No Homology to Known |
| | | | | Proteins |
| 127 | Gma.3969.2.S1_at | 2.246 | CD412522 | Energy |
| 128 | Gma.666.1.S1_at | 2.239 | AW310734 | Unclassified - Protein with |
| | | | | Unknown Function |
| 129 | GmaAffx.60984.1.S1_at | 2.234 | BU765206 | Transporter |
| 130 | Gma.3555.1.S1_at | 2.23 | BU764259 | Unclassified - Hypothetical |
| | | | | Protein NOT Supported by |
| | | | | cDNA |
| 131 | GmaAffx.56917.1.S1_at | 2.227 | AI441130 | Signal Transduction |
| 132 | GmaAffx.86059.1.S1_at | 2.206 | BF598759 | No Homology to Known |
| | | | | Proteins |
| 133 | GmaAffx.47891.1.S1_s_at | 2.197 | BQ785880 | Cell Structure |
| 134 | GmaAffx.37574.1.S1_s_at | 2.196 | BQ298504 | Secondary Metabolism |
| 135 | GmaAffx.13625.1.A1_at | 2.195 | BU547925 | No Homology to Known |
| | | | | Proteins |
| 136 | Gma.3777.1.S1_a_at | 2.191 | CD399954 | Unclassified - Hypothetical |
| | | | | Protein Supported by cDNA |
| 137 | Gma.11119.2.S1_s_at | 2.19 | CF920743 | Disease & Defense |
| 138 | GmaAffx.84266.1.S1_at | 2.19 | AI495798 | Signal Transduction |
| 139 | Gma.10573.3.S1_at | 2.185 | CA938507 | Transcription |
| 140 | GmaAffx.93561.1.S1_at | 2.178 | CF807977 | Metabolism |
| 141 | Gma.3777.1.S1_x_at | 2.178 | CD399954 | Unclassified - Hypothetical |
| | | | | Protein Supported by cDNA |
| 142 | Gma.5950.1.S1_s_at | 2.154 | BQ610563 | Disease & Defense |
| 143 | Gma.3751.1.S1_at | 2.134 | AW350303 | Unclassified - Hypothetical |
| | | | | Protein NOT Supported by |
| | | | | cDNA |
| 144 | GmaAffx.93214.1.S1_at | 2.117 | CF808939 | No Homology to Known |
| | | | | Proteins |
| 145 | Gma.10334.1.S1_at | 2.104 | AW234479 | Unclassified - Hypothetical |
| | | | | Protein NOT Supported by |
| | | | | cDNA |
| 146 | GmaAffx.78599.1.A1_at | 2.1 | CA784082 | No Homology to Known |
| | | | | Proteins |
| 147 | GmaAffx.10491.1.S1_at | 2.082 | AW598141 | No Homology to Known |
| | | | | Proteins |
| 148 | GmaAffx.77804.1.A1_at | 2.079 | BI968697 | No Homology to Known |
| | | | | Proteins |
| 149 | Gma.4437.1.S1_s_at | 2.079 | BQ628831 | Secondary Metabolism |
| 150 | Gma.6650.1.S1_at | 2.075 | BQ629283 | Unclassified - Protein with |
| | | | | Unknown Function |
| 151 | GmaAffx.90042.1.S1_at | 2.073 | CF805767 | Signal Transduction |
| 152 | GmaAffx.9618.1.S1_at | 2.067 | BM092432 | No Homology to Known |
| | | | | Proteins |
| 153 | Gma.17816.1.A1_at | 2.059 | BI970976 | Disease & Defense |
| 154 | Gma.2475.1.S1_at | 2.043 | BM522941 | Cell Structure |
| 155 | Gma.18084.1.S1_at | 2.026 | BI317557 | Disease & Defense |
| 156 | Gma.11257.1.S1_at | 2.016 | AW310476 | Cell Structure |
| 157 | Gma.6719.3.S1_s_at | 2.012 | AW164562 | Unclassified - Hypothetical |
| | | | | Protein NOT Supported by |
| | | | | cDNA |
| 158 | Gma.3189.2.S1_at | 2.005 | BG511492 | Signal Transduction |
| 159 | GmaAffx.74847.1.S1_at | 0.5 | BG507699 | Metabolism |
| 160 | Gma.6761.1.A1_at | 0.5 | BQ453407 | Cell Structure |
| 161 | GmaAffx.68754.1.S1_at | 0.499 | BE474746 | Secondary Metabolism |
| 162 | GmaAffx.84566.3.S1_s_at | 0.499 | BG045916 | Transcription |
| 163 | Gma.14338.1.A1_at | 0.499 | CF923111 | Protein Destination & Storage |
| 164 | GmaAffx.86247.1.S1_s_at | 0.499 | BG238674 | Unclassified - Hypothetical |
| | | | | Protein Supported by cDNA |
| 165 | Gma.4414.1.A1_at | 0.498 | BQ081678 | No Homology to Known |
| | | | | Proteins |
| 166 | Gma.8113.1.A1_at | 0.497 | BU548342 | No Homology to Known |
| | | | | Proteins |
| 167 | GmaAffx.15862.1.S1_at | 0.497 | BM092634 | Cell Structure |
| 168 | GmaAffx.52963.1.S1_at | 0.496 | AW310673 | Unclassified - Protein with |
| | | | | Unknown Function |
| 169 | GmaAffx.74674.1.S1_s_at | 0.496 | BU965464 | Transporter |
| 170 | GmaAffx.93220.1.S1_at | 0.496 | CF808945 | No Homology to Known |
| | | | | Proteins |
| 171 | GmaAffx.24350.1.A1_at | 0.495 | BU544060 | No Homology to Known |
| | | | | Proteins |
| 172 | GmaAffx.15983.2.S1_s_at | 0.495 | AW279540 | Signal Transduction |
| 173 | GmaAffx.35729.1.S1_s_at | 0.495 | BE806047 | Unclassified - Hypothetical |
| | | | | Protein Supported by cDNA |
| 174 | GmaAffx.90069.1.S1_s_at | 0.494 | CF805794 | Unclassified - Hypothetical |
| | | | | Protein Supported by cDNA |
| 175 | GmaAffx.83226.1.S1_at | 0.494 | CA852250 | Transcription |
| 176 | Gma.1622.1.A1_s_at | 0.493 | BU090523 | Signal Transduction |
| 177 | GmaAffx.85528.1.S1_at | 0.493 | BE608070 | Unclassified - Hypothetical |
| | | | | Protein Supported by cDNA |
| 178 | Gma.2252.1.S1_at | 0.492 | CD406598 | Unclassified - Hypothetical |
| | | | | Protein Supported by cDNA |
| 179 | Gma.9259.1.S1_at | 0.492 | CA782161 | Transporter |
| 180 | GmaAffx.92927.1.S1_at | 0.491 | CF805992 | No Homology to Known |
| | | | | Proteins |
| 181 | Gma.6895.1.S1_a_at | 0.491 | BU545788 | Metabolism |
| 182 | GmaAffx.57878.2.S1_at | 0.491 | BE610836 | Cell Structure |
| 183 | Gma.6375.1.S1_at | 0.489 | BQ610190 | Unclassified - Hypothetical |
| | | | | Protein NOT Supported by |
| | | | | cDNA |
| 184 | Gma.3332.1.S1_at | 0.489 | BQ298780 | Protein Destination & Storage |
| 185 | GmaAffx.92536.1.S1_s_at | 0.489 | CF808261 | Secondary Metabolism |
| 186 | Gma.17638.2.S1_at | 0.489 | BI786260 | Transcription |
| 187 | Gma.17610.1.S1_x_at | 0.488 | U50075.1 | Metabolism |
| 188 | Gma.5701.3.S1_at | 0.488 | CA852693 | No Homology to Known |
| | | | | Proteins |
| 189 | GmaAffx.81999.1.S1_at | 0.487 | AI416637 | Transcription |
| 190 | GmaAffx.83880.1.A1_at | 0.486 | BE822640 | Disease & Defense |
| 191 | GmaAffx.55657.1.S1_at | 0.486 | BM885600 | No Homology to Known |
| | | | | Proteins |
| 192 | GmaAffx.57967.1.S1_at | 0.486 | AW102170 | Protein Destination & Storage |
| 193 | Gma.12987.1.S1_at | 0.485 | CD392747 | Transcription |
| 194 | Gma.13218.1.A1_s_at | 0.484 | CD391892 | No Homology to Known |
| | | | | Proteins |
| 195 | GmaAffx.60479.1.S1_at | 0.484 | BG726023 | Transcription |
| 196 | GmaAffx.50613.1.A1_at | 0.484 | BI967418 | No Homology to Known |
| | | | | Proteins |
| 197 | Gma.3589.1.S1_a_at | 0.483 | AW101438 | Secondary Metabolism |
| 198 | Gma.1573.2.S1_a_at | 0.483 | BI974098 | Metabolism |
| 199 | GmaAffx.23789.1.S1_at | 0.482 | BU551014 | Unclassified - Hypothetical |
| | | | | Protein Supported by cDNA |
| 200 | Gma.221.1.S1_at | 0.481 | U41475.1 | Metabolism |
| 201 | GmaAffx.55124.1.S1_at | 0.481 | BI970821 | Unclassified - Hypothetical |
| | | | | Protein Supported by cDNA |
| 202 | Gma.6643.1.A1_at | 0.481 | BE823205 | Secondary Metabolism |
| 203 | Gma.17610.1.S1_at | 0.481 | U50075.1 | Metabolism |
| 204 | GmaAffx.13039.1.A1_at | 0.481 | BF219584 | Secondary Metabolism |
| 205 | GmaAffx.53845.1.S1_at | 0.48 | BG406552 | Transporter |
| 206 | Gma.5294.1.S1_at | 0.479 | BE658252 | Unclassified - Hypothetical |
| | | | | Protein Supported by cDNA |
| 207 | GmaAffx.73857.1.S1_at | 0.479 | BM885538 | Unclassified - Hypothetical |
| | | | | Protein Supported by cDNA |
| 208 | Gma.17798.2.S1_at | 0.479 | CD412748 | No Homology to Known |
| | | | | Proteins |
| 209 | Gma.13273.1.A1_at | 0.478 | CD397107 | Unclassified - Hypothetical |
| | | | | Protein Supported by cDNA |
| 210 | GmaAffx.47451.1.S1_at | 0.478 | CA799081 | Unclassified - Hypothetical |
| | | | | Protein NOT Supported by |
| | | | | cDNA |
| 211 | GmaAffx.90234.1.S1_at | 0.477 | CF805959 | Transcription |
| 212 | GmaAffx.6635.1.S1_at | 0.477 | CD412718 | Disease & Defense |
| 213 | GmaAffx.14141.1.A1_at | 0.476 | BI973009 | No Homology to Known |
| | | | | Proteins |
| 214 | Gma.6129.2.S1_at | 0.475 | AW186006 | Protein Synthesis |
| 215 | GmaAffx.499.1.S1_at | 0.475 | BE058375 | Transcription |
| 216 | Gma.2565.1.S1_at | 0.475 | BG652533 | Unclassified - Hypothetical |
| | | | | Protein Supported by cDNA |
| 217 | Gma.5331.1.S1_aat | 0.475 | BI892702 | Transcription |
| 218 | GmaAffx.84338.1.S1_at | 0.474 | AI856789 | Unclassified - Hypothetical |
| | | | | Protein Supported by cDNA |
| 219 | Gma.3519.1.S1_at | 0.474 | CD403581 | Unclassified - Hypothetical |
| | | | | Protein Supported by cDNA |
| 220 | Gma.13035.1.S1_at | 0.474 | CD396536 | No Homology to Known |
| | | | | Proteins |
| 221 | Gma.17502.2.S1_x_at | 0.473 | BG046201 | Transcription |
| 222 | Gma.6310.1.S1_at | 0.472 | BQ612831 | Signal Transduction |
| 223 | GmaAffx.73059.1.S1_at | 0.472 | BI426008 | Unclassified - Hypothetical |
| | | | | Protein NOT Supported by |
| | | | | cDNA |
| 224 | GmaAffx.93430.1.S1_s_at | 0.47 | CF809155 | Disease & Defense |
| 225 | GmaAffx.15471.1.S1_at | 0.47 | BQ611747 | Transcription |
| 226 | GmaAffx.74256.1.S1_at | 0.469 | BE057293 | No Homology to Known |
| | | | | Proteins |
| 227 | GmaAffx.18376.1.A1_at | 0.469 | BI321742 | Protein Destination & Storage |
| 228 | GmaAffx.69198.1.S1_at | 0.468 | BM526951 | Unclassified - Hypothetical |
| | | | | Protein Supported by cDNA |
| 229 | GmaAffx.33527.1.S1_at | 0.468 | BU761195 | No Homology to Known |
| | | | | Proteins |
| 230 | Gma.13800.1.A1_at | 0.468 | CD405906 | Unclassified - Hypothetical |
| | | | | Protein NOT Supported by |
| | | | | cDNA |
| 231 | GmaAffx.78561.1.S1_at | 0.467 | BQ452483 | Metabolism |
| 232 | Gma.7040.1.A1_at | 0.467 | BE346424 | Transcription |
| 233 | GmaAffx.26320.1.S1_at | 0.466 | BE440416 | Unclassified - Hypothetical |
| | | | | Protein Supported by cDNA |
| 234 | GmaAffx.494.1.S1_at | 0.464 | AI938330 | Cell Structure |
| 235 | Gma.4095.1.S1_at | 0.463 | AW350037 | Unclassified - Hypothetical |
| | | | | Protein Supported by cDNA |
| 236 | GmaAffx.78297.1.S1_at | 0.463 | BM731185 | Unclassified - Hypothetical |
| | | | | Protein Supported by cDNA |
| 237 | GmaAffx.93116.1.S1_at | 0.463 | CF806290 | Disease & Defense |
| 238 | GmaAffx.47288.1.S1_at | 0.461 | BU764979 | Unclassified - Hypothetical |
| | | | | Protein Supported by cDNA |
| 239 | GmaAffx.15204.1.A1_at | 0.461 | BU549835 | No Homology to Known |
| | | | | Proteins |
| 240 | Gma.6213.1.S1_at | 0.461 | CD401652 | Protein Destination & Storage |
| 241 | GmaAffx.35908.1.S1_at | 0.46 | BF598958 | No Homology to Known |
| | | | | Proteins |
| 242 | Gma.2780.1.A1_at | 0.458 | AI988066 | Unclassified - Hypothetical |
| | | | | Protein NOT Supported by |
| | | | | cDNA |
| 243 | Gma.3578.1.S1_s_at | 0.458 | AW156522 | Disease & Defense |
| 244 | GmaAffx.50673.2.S1_s_at | 0.458 | BI787147 | Transcription |
| 245 | GmaAffx.23292.1.A1_at | 0.457 | BI967795 | No Homology to Known |
| | | | | Proteins |
| 246 | GmaAffx.91143.1.S1_at | 0.457 | CF806868 | Signal Transduction |
| 247 | Gma.1619.1.S1_at | 0.457 | AW472195 | Unclassified - Hypothetical |
| | | | | Protein Supported by cDNA |
| 248 | Gma.17070.1.S1_at | 0.456 | BE822463 | Disease & Defense |
| 249 | Gma.11026.2.S1_s_at | 0.455 | BQ080041 | Disease & Defense |
| 250 | GmaAffx.90899.1.S1_at | 0.454 | CF806624 | Unclassified - Hypothetical |
| | | | | Protein NOT Supported by |
| | | | | cDNA |
| 251 | Gma.8490.1.S1_at | 0.454 | AB030492.1 | Metabolism |
| 252 | GmaAffx.84964.1.S1_at | 0.453 | AW598267 | No Homology to Known |
| | | | | Proteins |
| 253 | GmaAffx.76699.1.S1_at | 0.451 | AW760679 | Transcription |
| 254 | Gma.8370.1.S1_at | 0.45 | BQ454087 | Signal Transduction |
| 255 | GmaAffx.41990.1.S1_at | 0.449 | CA936708 | No Homology to Known |
| | | | | Proteins |
| 256 | GmaAffx.32700.1.S1_at | 0.449 | BM522940 | No Homology to Known |
| | | | | Proteins |
| 257 | Gma.9205.1.S1_at | 0.447 | BM528605 | Unclassified - Hypothetical |
| | | | | Protein Supported by cDNA |
| 258 | Gma.4400.1.S1_at | 0.447 | BI317776 | Transcription |
| 259 | GmaAffx.45003.1.S1_at | 0.445 | BI968048 | Unclassified - Hypothetical |
| | | | | Protein Supported by cDNA |
| 260 | Gma.6033.2.S1_at | 0.444 | AI416728 | Unclassified - Hypothetical |
| | | | | Protein Supported by cDNA |
| 261 | GmaAffx.73452.2.S1_at | 0.444 | BM085321 | Signal Transduction |
| 262 | Gma.1929.1.S1_at | 0.443 | BU083650 | Transcription |
| 263 | GmaAffx.49777.1.S1_at | 0.443 | BG047001 | No Homology to Known |
| | | | | Proteins |
| 264 | Gma.11205.1.S1_a_at | 0.442 | BF066553 | Metabolism |
| 265 | GmaAffx.34798.1.S1_at | 0.442 | BM143105 | Unclassified - Hypothetical |
| | | | | Protein Supported by cDNA |
| 266 | GmaAffx.69369.1.S1_at | 0.442 | BG511072 | Unclassified - Hypothetical |
| | | | | Protein Supported by cDNA |
| 267 | Gma.6192.1.S1_s_at | 0.44 | AW396773 | Metabolism |
| 268 | GmaAffx.60057.1.S1_at | 0.439 | BM308158 | No Homology to Known |
| | | | | Proteins |
| 269 | Gma.6151.1.S1_at | 0.438 | BQ611806 | Metabolism |
| 270 | GmaAffx.21041.1.A1_at | 0.438 | AW350819 | No Homology to Known |
| | | | | Proteins |
| 271 | Gma.13030.1.S1_a_at | 0.437 | AI748012 | Protein Destination & Storage |
| 272 | GmaAffx.90600.1.S1_at | 0.437 | CF806325 | Metabolism |
| 273 | GmaAffx.59808.1.S1_at | 0.435 | BM886228 | Transporter |
| 274 | Gma.2776.1.S1_at | 0.434 | BI968513 | Secondary Metabolism |
| 275 | GmaAffx.15983.2.S1_at | 0.434 | AW279540 | Signal Transduction |
| 276 | Gma.7788.1.A1_at | 0.434 | BU549148 | Unclassified - Hypothetical |
| | | | | Protein Snpported by cDNA |
| 277 | GmaAffx.11049.1.S1_at | 0.432 | BU545140 | Unclassified - Protein with |
| | | | | Unknown Function |
| 278 | Gma.2776.2.S1_s_at | 0.432 | BU577475 | Secondary Metabolism |
| 279 | GmaAffx.91807.1.S1_at | 0.432 | CF807532 | No Homology to Known |
| | | | | Proteins |
| 280 | GmaAffx.87417.1.S1_at | 0.43 | BM568072 | Metabolism |
| 281 | GmaAffx.48042.1.S1_at | 0.429 | AI960210 | Transporter |
| 282 | GmaAffx.85999.1.S1_at | 0.429 | BF426048 | Unclassified - Hypothetical |
| | | | | Protein Supported by cDNA |
| 283 | Gma.3963.2.S1_at | 0.429 | BE608192 | Unclassified - Hypothetical |
| | | | | Protein NOT Supported by |
| | | | | cDNA |
| 284 | GmaAffx.12014.1.A1_at | 0.428 | CA802431 | Unclassified - Hypothetical |
| | | | | Protein Supported by cDNA |
| 285 | GmaAffx.59210.1.S1_at | 0.427 | BI967182 | Secondary Metabolism |
| 286 | Gma.1243.1.S1_at | 0.426 | AW311027 | Unclassified - Hypothetical |
| | | | | Protein Supported by cDNA |
| 287 | Gma.3450.1.S1_at | 0.425 | BE657895 | Unclassified - Hypothetical |
| | | | | Protein Supported by cDNA |
| 288 | Gma.5159.1.A1_at | 0.425 | BI971147 | No Homology to Known |
| | | | | Proteins |
| 289 | GmaAffx.15471.3.S1_x_at | 0.425 | BE346777 | Transcription |
| 290 | GmaAffx.9280.1.S1_at | 0.425 | BI498912 | Signal Transduction |
| 291 | Gma.1661.1.S1_at | 0.424 | BE821382 | Metabolism |
| 292 | GmaAffx.91428.1.A1_at | 0.423 | CF807137 | Metabolism |
| 293 | Gma.1061.1.A1_at | 0.421 | CD408183 | Metabolism |
| 294 | GmaAffx.59014.1.S1_s_at | 0.421 | AW733977 | No Homology to Known |
| | | | | Proteins |
| 295 | Gma.10536.1.S1_at | 0.42 | BG508300 | No Homology to Known |
| | | | | Proteins |
| 296 | Gma.5478.1.S1_at | 0.419 | AW350606 | Metabolism |
| 297 | GmaAffx.4036.1.S1_at | 0.419 | BM527313 | Unclassified - Hypothetical |
| | | | | Protein Supported by cDNA |
| 298 | Gma.15468.1.S1_at | 0.418 | BE800435 | Disease & Defense |
| 299 | Gma.1495.1.S1_at | 0.418 | BI973545 | No Homology to Known |
| | | | | Proteins |
| 300 | GmaAffx.53099.1.S1_at | 0.418 | BG043340 | Unclassified - Hypothetical |
| | | | | Protein NOT Supported by |
| | | | | cDNA |
| 301 | GmaAffx.85287.2.S1_at | 0.417 | BI472203 | Unclassified - Hypothetical |
| | | | | Protein Supported by cDNA |
| 302 | Gma.5586.2.S1_at | 0.416 | CD398384 | Transcription |
| 303 | GmaAffx.81502.1.S1_at | 0.415 | CD404946 | Unclassified - Hypothetical |
| | | | | Protein NOT Supported by |
| | | | | cDNA |
| 304 | GmaAffx.83016.1.S1_at | 0.415 | BQ080317 | Transcription |
| 305 | Gma.1445.1.S1_at | 0.414 | BQ454241 | Metabolism |
| 306 | Gma.3384.1.S1_at | 0.414 | BE659400 | Unclassified - Protein with |
| | | | | Unknown Function |
| 307 | GmaAffx.948.1.S1_at | 0.414 | BG155389 | Protein Destination & Storage |
| 308 | Gma.6684.1.S1_at | 0.413 | AW459859 | Signal Transduction |
| 309 | Gma.3963.1.S1_at | 0.413 | AW202007 | Unclassified - Hypothetical |
| | | | | Protein NOT Supported by |
| | | | | cDNA |
| 310 | GmaAffx.8999.2.A1_s_at | 0.413 | BI700574 | No Homology to Known |
| | | | | Proteins |
| 311 | Gma.4687.1.S1_at | 0.412 | AW309716 | Secondary Metabolism |
| 312 | Gma.6192.1.S1_at | 0.411 | AW396773 | Metabolism |
| 313 | GmaAffx.81287.1.S1_at | 0.411 | BG789983 | Secondary Metabolism |
| 314 | Gma.3591.2.S1_at | 0.409 | BE473658 | Secondary Metabolism |
| 315 | GmaAffx.29616.1.A1_at | 0.409 | CD404723 | No Homology to Known |
| | | | | Proteins |
| 316 | GmaAffx.74160.1.S1_at | 0.409 | BQ740845 | Protein Destination & Storage |
| 317 | Gma.10969.3.S1_x_at | 0.407 | CD396552 | Metabolism |
| 318 | Gma.16910.1.S1_at | 0.406 | BU549219 | Unclassified - Protein with |
| | | | | Unknown Function |
| 319 | Gma.1758.1.S1_at | 0.406 | CD402046 | Unclassified - Protein with |
| | | | | Unknown Function |
| 320 | PsAffx.C107000004_at | 0.402 | | |
| 321 | Gma.3914.1.S1_at | 0.401 | BI702206 | Unclassified - Hypothetical |
| | | | | Protein Supported by cDNA |
| 322 | Gma.1394.1.S1_at | 0.4 | BG839300 | Metabolism |
| 323 | Gma.8489.1.S1_s_at | 0.4 | AB030493.1 | Metabolism |
| 324 | GmaAffx.42732.1.S1_at | 0.399 | BU765857 | Protein Destination & Storage |
| 325 | GmaAffx.93342.1.S1_s_at | 0.399 | CF809067 | Disease & Defense |
| 326 | GmaAffx.61777.1.S1_at | 0.399 | AI965744 | Signal Transduction |
| 327 | GmaAffx.70005.1.S1_s_at | 0.395 | CA936335 | Secondary Metabolism |
| 328 | Gma.1951.2.S1_a_at | 0.392 | CA783485 | Protein Destination & Storage |
| 329 | GmaAffx.77203.1.S1_at | 0.39 | BI970687 | Unclassified - Hypothetical |
| | | | | Protein Supported by cDNA |
| 330 | Gma.1394.2.S1_at | 0.389 | BI470370 | Metabolism |
| 331 | Gma.4252.1.S1_at | 0.389 | BE821208 | Metabolism |
| 332 | GmaAffx.14153.2.S1_at | 0.387 | BE329834 | Unclassified - Hypothetical |
| | | | | Protein Supported by cDNA |
| 333 | Gma.3578.1.S1_at | 0.386 | AW156522 | Disease & Defense |
| 334 | Gma.8946.1.S1_at | 0.386 | BQ453813 | Transcription |
| 335 | GmaAffx.34449.1.S1_at | 0.386 | AW164447 | No Homology to Known |
| | | | | Proteins |
| 336 | Gma.3420.1.S1_at | 0.385 | CD406082 | Unclassified - Hypothetical |
| | | | | Protein Supported by cDNA |
| 337 | Gma.4150.2.S1_s_at | 0.384 | AW350622 | Unclassified - Hypothetical |
| | | | | Protein Supported by cDNA |
| 338 | GmaAffx.15196.1.S1_at | 0.384 | BU549436 | Metabolism |
| 339 | Gma.16359.1.A1_at | 0.384 | BI970359 | Metabolism |
| 340 | GmaAffx.29951.2.S1_at | 0.383 | AW100899 | Metabolism |
| 341 | Gma.6033.1.S1_at | 0.382 | BI967767 | Unclassified - Hypothetical |
| | | | | Protein Supported by cDNA |
| 342 | Gma.3987.2.S1_a_at | 0.381 | BI971594 | No Homology to Known |
| | | | | Proteins |
| 343 | GmaAffx.12142.1.S1_at | 0.38 | AW733336 | Metabolism |
| 344 | GmaAffx.57432.2.S1_at | 0.38 | CA819434 | No Homology to Known |
| | | | | Proteins |
| 345 | GmaAffx.90600.1.S1_s_at | 0.379 | CF806325 | Metabolism |
| 346 | GmaAffx.93212.1.S1_s_at | 0.379 | CF807524 | Transporter |
| 347 | GmaAffx.61311.1.S1_at | 0.378 | CA936951 | Transporter |
| 348 | Gma.2540.2.S1_s_at | 0.378 | BI787366 | No Homology to Known |
| | | | | Proteins |
| 349 | Gma.4099.1.S1_at | 0.378 | BG651846 | Metabolism |
| 350 | Gma.8262.1.A1_at | 0.377 | BU550168 | Signal Transduction |
| 351 | Gma.17402.1.A1_at | 0.376 | BE611084 | No Homology to Known |
| | | | | Proteins |
| 352 | Gma.4593.1.S1_a_at | 0.373 | CD396298 | Energy |
| 353 | Gma.2388.1.S1_at | 0.372 | CD416539 | No Homology to Known |
| | | | | Proteins |
| 354 | Gma.13418.3.S1_at | 0.372 | CA938391 | Transporter |
| 355 | Gma.1326.1.S1_at | 0.37 | CD397515 | Secondary Metabolism |
| 356 | Gma.7227.1.S1_at | 0.37 | BU551112 | Protein Destination & Storage |
| 357 | GmaAffx.78358.2.S1_at | 0.37 | BU762686 | Metabolism |
| 358 | Gma.1490.1.S1_at | 0.369 | AB000097.1 | Cell Structure |
| 359 | GmaAffx.58125.1.A1_at | 0.368 | BE347561 | Transcription |
| 360 | GmaAffx.36470.1.A1_at | 0.367 | BU550552 | Unclassified - Hypothetical |
| | | | | Protein Supported by cDNA |
| 361 | Gma.7831.1.S1_s_at | 0.367 | CD393602 | Transporter |
| 362 | GmaAffx.87900.1.S1_at | 0.367 | BI892943 | Protein Destination & Storage |
| 363 | Gma.5513.1.S1_at | 0.367 | AW310453 | Metabolism |
| 364 | Gma.1164.1.S1_at | 0.366 | BE822845 | Energy |
| 365 | Gma.17323.1.S1_at | 0.366 | AW567763 | No Homology to Known |
| | | | | Proteins |
| 366 | GmaAffx.91731.1.S1_s_at | 0.363 | CF807456 | Secondary Metabolism |
| 367 | Gma.2066.1.S1_a_at | 0.362 | BG363704 | No Homology to Known |
| | | | | Proteins |
| 368 | Gma.11166.1.S1_x_at | 0.359 | AW705829 | Metabolism |
| 369 | GmaAffx.71308.1.S1_at | 0.358 | BF595112 | Transcription |
| 370 | GmaAffx.82936.1.S1_at | 0.358 | AI938364 | Unclassified - Protein with |
| | | | | Unknown Function |
| 371 | GmaAffx.93328.1.S1_s_at | 0.357 | CF809053 | Secondary Metabolism |
| 372 | GmaAffx.93591.1.S1_s_at | 0.354 | CF809316 | Metabolism |
| 373 | GmaAffx.64807.1.S1_at | 0.354 | AW201972 | No Homology to Known |
| | | | | Proteins |
| 374 | Gma.857.1.S1_at | 0.352 | BE822264 | No Homology to Known |
| | | | | Proteins |
| 375 | Gma.7580.1.S1_at | 0.351 | BE057763 | Unclassified - Hypothetical |
| | | | | Protein Supported by cDNA |
| 376 | Gma.3591.1.S1_a_at | 0.35 | BE440266 | Secondary Metabolism |
| 377 | Gma.10216.3.A1_x_at | 0.348 | BI969715 | Metabolism |
| 378 | Gma.5162.1.A1_at | 0.347 | BI971156 | Signal Transduction |
| 379 | GmaAffx.17892.2.S1_at | 0.345 | BQ628677 | No Homology to Known |
| | | | | Proteins |
| 380 | Gma.11166.1.S1_s_at | 0.344 | AW705829 | Metabolism |
| 381 | GmaAffx.54778.1.S1_at | 0.344 | BI322039 | No Homology to Known |
| | | | | Proteins |
| 382 | Gma.9647.1.S1_at | 0.343 | BE820997 | Cell Structure |
| 383 | GmaAffx.11242.1.S1_s_at | 0.343 | AW317461 | Protein Destination & Storage |
| 384 | GmaAffx.80584.1.S1_at | 0.343 | BU083387 | Transcription |
| 385 | Gma.9740.1.S1_at | 0.342 | BQ081058 | Unclassified - Hypothetical |
| | | | | Protein Supported by cDNA |
| 386 | Gma.5760.2.S1_at | 0.341 | BQ298381 | Disease & Defense |
| 387 | Gma.5929.1.S1_at | 0.341 | BQ610446 | No Homology to Known |
| | | | | Proteins |
| 388 | Gma.13150.1.A1_at | 0.341 | CD411577 | Unclassified - Protein with |
| | | | | Unknown Function |
| 389 | GmaAffx.53324.1.A1_at | 0.341 | AW309251 | Transcription |
| 390 | Gma.4643.1.S1_at | 0.339 | BU547906 | Transcription |
| 391 | Gma.398.2.S1_at | 0.338 | BU925900 | Metabolism |
| 392 | GmaAffx.92791.1.S1_at | 0.336 | CF808516 | Metabolism |
| 393 | Gma.17516.1.S1_at | 0.336 | CD396417 | Transporter |
| 394 | GmaAffx.60479.2.S1_at | 0.334 | BU765815 | Transcription |
| 395 | GmaAffx.68507.1.S1_at | 0.333 | BI699405 | Transcription |
| 396 | Gma.16500.1.S1_at | 0.332 | BE059054 | Metabolism |
| 397 | GmaAffx.32011.1.S1_at | 0.332 | BI701198 | No Homology to Known |
| | | | | Proteins |
| 398 | GmaAffx.83715.1.S1_at | 0.331 | BI785710 | Transporter |
| 399 | GmaAffx.11897.1.S1_at | 0.33 | BE819868 | Transporter |
| 400 | Gma.4728.1.A1_at | 0.328 | BI968944 | Secondary Metabolism |
| 401 | Gma.15307.1.S1_at | 0.326 | U64866.1 | Disease & Defense |
| 402 | GmaAffx.79516.1.A1_at | 0.326 | CD400596 | Transcription |
| 403 | GmaAffx.64967.1.S1_at | 0.325 | BU546115 | Secondary Metabolism |
| 404 | Gma.1951.1.S1_at | 0.321 | BQ079657 | Protein Destination & Storage |
| 405 | GmaAffx.63464.1.S1_at | 0.318 | BG156739 | No Homology to Known |
| | | | | Proteins |
| 406 | Gma.10591.2.S1_at | 0.318 | CD414199 | Metabolism |
| 407 | GmaAffx.61130.1.S1_at | 0.314 | CA799668 | Transporter |
| 408 | Gma.1314.1.S1_at | 0.314 | U63726.1 | Metabolism |
| 409 | GmaAffx.88546.1.S1_at | 0.313 | BE801134 | Metabolism |
| 410 | GmaAffx.64794.1.S1_at | 0.313 | CA801749 | Unclassified - Hypothetical |
| | | | | Protein Supported by cDNA |
| 411 | Gma.1394.2.S1_a_at | 0.313 | BI470370 | Metabolism |
| 412 | Gma.12227.1.S1_at | 0.312 | BG511671 | Unclassified - Hypothetical |
| | | | | Protein NOT Supported by |
| | | | | cDNA |
| 413 | GmaAffx.32011.2.S1_at | 0.312 | BM084966 | Transcription |
| 414 | Gma.177.1.S1_at | 0.312 | AF004806.1 | Protein Destination & Storage |
| 415 | Gma.8022.1.S1_at | 0.311 | BQ628998 | Disease & Defense |
| 416 | GmaAffx.27331.1.S1_at | 0.31 | BM520297 | Unclassified - Hypothetical |
| | | | | Protein NOT Supported by |
| | | | | cDNA |
| 417 | GmaAffx.33677.1.S1_at | 0.308 | CA782582 | No Homology to Known |
| | | | | Proteins |
| 418 | GmaAffx.66215.1.S1_at | 0.308 | AI443109 | Signal Transduction |
| 419 | GmaAffx.77832.1.S1_at | 0.305 | BI971626 | Unclassified - Hypothetical |
| | | | | Protein NOT Supported by |
| | | | | cDNA |
| 420 | PsAffx.psMA015xP13f_at | 0.305 | | |
| 421 | GmaAffx.17961.1.A1_at | 0.303 | BE657601 | Protein Destination & Storage |
| 422 | Gma.728.1.A1_at | 0.3 | BE658779 | No Homology to Known |
| | | | | Proteins |
| 423 | GmaAffx.31640.1.S1_at | 0.3 | BG237979 | No Homology to Known |
| | | | | Proteins |
| 424 | Gma.4874.1.A1_at | 0.298 | AI938293 | No Homology to Known |
| | | | | Proteins |
| 425 | Gma.11028.2.S1_s_at | 0.297 | CA782711 | Metabolism |
| 426 | GmaAffx.50980.1.S1_s_at | 0.296 | BE823095 | Signal Transduction |
| 427 | GmaAffx.26767.1.S1_at | 0.295 | BF010276 | Transcription |
| 428 | Gma.5513.1.S1_x_at | 0.295 | AW310453 | Metabolism |
| 429 | GmaAffx.78163.1.S1_s_at | 0.293 | BM520857 | Signal Transduction |
| 430 | GmaAffx.11172.1.A1_at | 0.293 | CD404556 | No Homology to Known |
| | | | | Proteins |
| 431 | Gma.8530.1.S1_at | 0.292 | AF109720.1 | Protein Destination & Storage |
| 432 | GmaAffx.5255.1.S1_at | 0.292 | BG362929 | Transcription |
| 433 | Gma.3118.1.S1_s_at | 0.29 | BQ786048 | Disease & Defense |
| 434 | Gma.8058.1.A1_at | 0.289 | BM271479 | Signal Transduction |
| 435 | Gma.7437.1.S1_at | 0.289 | BU547705 | Unclassified - Hypothetical |
| | | | | Protein Supported by cDNA |
| 436 | GmaAffx.85382.1.S1_s_at | 0.289 | BI945154 | No Homology to Known |
| | | | | Proteins |
| 437 | Gma.3744.2.S1_a_at | 0.288 | BI892693 | Unclassified - Protein With |
| | | | | Unknown Function |
| 438 | GmaAffx.56319.1.S1_at | 0.288 | BE802287 | Metabolism |
| 439 | Gma.13452.1.A1_at | 0.287 | CD398445 | No Homology to Known |
| | | | | Proteins |
| 440 | GmaAffx.58712.1.S1_at | 0.287 | BF325034 | Transporter |
| 441 | GmaAffx.3369.2.A1_at | 0.286 | BU550316 | Cell Structure |
| 442 | Gma.17688.1.S1_s_at | 0.285 | CA937360 | Transcription |
| 443 | Gma.5469.1.S1_at | 0.284 | AW349200 | No Homology to Known |
| | | | | Proteins |
| 444 | GmaAffx.89624.1.A1_s_at | 0.279 | CK606100 | Transcription |
| 445 | Gma.649.1.S1_at | 0.274 | AW568984 | No Homology to Known |
| | | | | Proteins |
| 446 | GmaAffx.90395.1.A1_s_at | 0.274 | CF807499 | Unclassified - Protein with |
| | | | | Unknown Function |
| 447 | Gma.595.1.S1_at | 0.273 | AB004271.1 | Metabolism |
| 448 | GmaAffx.59252.1.A1_at | 0.272 | AI442669 | Protein Destination & Storage |
| 449 | Gma.3744.1.S1_at | 0.27 | CD417438 | Unclassified - Protein With |
| | | | | Unknown Function |
| 450 | GmaAffx.70435.2.S1_at | 0.266 | AW705853 | Transcription |
| 451 | Gma.14471.1.S1_s_at | 0.265 | CF921241 | No Homology to Known |
| | | | | Proteins |
| 452 | Gma.2253.1.S1_at | 0.264 | AF169018.1 | Metabolism |
| 453 | Gma.12731.2.A1_s_at | 0.263 | CD395848 | Unclassified - Hypothetical |
| | | | | Protein NOT Supported by |
| | | | | cDNA |
| 454 | GmaAffx.34657.1.A1_s_at | 0.261 | AW351012 | No Homology to Known |
| | | | | Proteins |
| 455 | Gma.2086.1.S1_at | 0.258 | AF035671.1 | Metabolism |
| 456 | GmaAffx.34975.1.S1_at | 0.256 | BU547817 | Unclassified - Hypothetical |
| | | | | Protein Supported by cDNA |
| 457 | Gma.10227.1.A1_at | 0.253 | BE020065 | Metabolism |
| 458 | Gma.10896.3.A1_at | 0.253 | BI316837 | Metabolism |
| 459 | Gma.968.1.S1_at | 0.253 | D28876.1 | Protein Destination & Storage |
| 460 | GmaAffx.26908.1.S1_at | 0.253 | BF425834 | Secondary Metabolism |
| 461 | GmaAffx.25612.1.S1_at | 0.252 | AW317577 | No Homology to Known |
| | | | | Proteins |
| 462 | GmaAffx.84424.1.S1_at | 0.252 | AW311543 | Metabolism |
| 463 | GmaAffx.17918.1.S1_at | 0.25 | BG157024 | No Homology to Known |
| | | | | Proteins |
| 464 | Gma.10904.1.S1_s_at | 0.247 | CD408184 | Protein Destination & Storage |
| 465 | GmaAffx.2710.1.S1_at | 0.247 | BE822538 | Unclassified - Hypothetical |
| | | | | Protein Supported by cDNA |
| 466 | GmaAffx.45957.2.S1_at | 0.247 | BI974135 | No Homology to Known |
| | | | | Proteins |
| 467 | GmaAffx.11815.1.S1_at | 0.246 | AW311637 | Metabolism |
| 468 | Gma.11731.1.A1_at | 0.243 | CD392320 | Unclassified - Protein with |
| | | | | Unknown Function |
| 469 | GmaAffx.32606.1.S1_at | 0.241 | BM270707 | Unclassified - Hypothetical |
| | | | | Protein NOT Supported by |
| | | | | cDNA |
| 470 | GmaAffx.80188.1.A1_at | 0.239 | AW349832 | No Homology to Known |
| | | | | Proteins |
| 471 | GmaAffx.57970.1.S1_at | 0.238 | BM886862 | Transcription |
| 472 | GmaAffx.40923.1.S1_at | 0.238 | BG653848 | Disease & Defense |
| 473 | GmaAffx.52826.3.S1_at | 0.237 | BF596503 | Signal Transduction |
| 474 | GmaAffx.10582.1.S1_at | 0.234 | BM523687 | Transcription |
| 475 | GmaAffx.82263.1.S1_s_at | 0.234 | AW568454 | No Homology to Known |
| | | | | Proteins |
| 476 | Gma.12068.1.A1_at | 0.232 | BU547739 | Transporter |
| 477 | GmaAffx.71308.2.A1_at | 0.23 | AW569888 | No Homology to Known |
| | | | | Proteins |
| 478 | GmaAffx.63054.1.S1_at | 0.229 | BE806784 | Unclassified - Hypothetical |
| | | | | Protein NOT Supported by |
| | | | | cDNA |
| 479 | Gma.12684.1.S1_at | 0.226 | CD394773 | Transcription |
| 480 | GmaAffx.19118.1.S1_at | 0.225 | BM523731 | Unclassified - Hypothetical |
| | | | | Protein NOT Supported by |
| | | | | cDNA |
| 481 | Gma.1527.1.S1_x_at | 0.22 | BI968185 | Secondary Metabolism |
| 482 | Gma.5331.1.S1_at | 0.22 | BI892702 | Transcription |
| 483 | GmaAffx.30792.1.A1_at | 0.22 | BU544815 | Unclassified - Hypothetical |
| | | | | Protein Supported by cDNA |
| 484 | Gma.17688.1.S1_at | 0.219 | CA937360 | Transcription |
| 485 | Gma.7824.1.S1_at | 0.219 | BU550492 | Metabolism |
| 486 | Gma.1768.1.S1_s_at | 0.219 | BE820777 | Unclassified - Hypothetical |
| | | | | Protein Supported by cDNA |
| 487 | GmaAffx.81362.1.S1_at | 0.218 | BF324988 | Cell Structure |
| 488 | GmaAffx.38555.2.S1_at | 0.214 | BI425873 | No Homology to Known |
| | | | | Proteins |
| 489 | Gma.11873.1.S1_at | 0.207 | CA819977 | Unclassified - Protein with |
| | | | | Unknown Function |
| 490 | Gma.2887.2.S1_x_at | 0.206 | BI701520 | Secondary Metabolism |
| 491 | Gma.17985.2.S1_at | 0.205 | BE802263 | Metabolism |
| 492 | GmaAffx.90113.1.S1_at | 0.202 | CF806004 | Protein Destination & Storage |
| 493 | GmaAffx.26868.1.S1_at | 0.201 | BF325443 | Unclassified - Hypothetical |
| | | | | Protein NOT Supported by |
| | | | | cDNA |
| 494 | GmaAffx.91338.1.S1_at | 0.2 | CF807063 | Unclassified - Hypothetical |
| | | | | Protein Supported by cDNA |
| 495 | GmaAffx.50980.1.S1_x_at | 0.199 | BE823095 | Signal Transduction |
| 496 | Gma.13854.1.S1_at | 0.199 | CD408166 | Metabolism |
| 497 | GmaAffx.50980.1.S1_at | 0.197 | BE823095 | Signal Transduction |
| 498 | GmaAffx.31862.1.S1_at | 0.192 | BE822501 | Unclassified - Hypothetical |
| | | | | Protein Supported by cDNA |
| 499 | GmaAffx.93212.1.S1_at | 0.191 | CF807524 | Transporter |
| 500 | GmaAffx.90395.1.S1_s_at | 0.186 | CF806120 | Unclassified - Protein with |
| | | | | Unknown Function |
| 501 | Gma.15.1.S1_at | 0.185 | U66316.1 | Unclassified - Protein with |
| | | | | Unknown Function |
| 502 | GmaAffx.9911.1.S1_at | 0.184 | BU547808 | Unclassified - Hypothetical |
| | | | | Protein NOT Supported by |
| | | | | cDNA |
| 503 | GmaAffx.81851.1.S1_at | 0.181 | AW706751 | Unclassified - Hypothetical |
| | | | | Protein Supported by cDNA |
| 504 | GmaAffx.12665.1.A1_at | 0.176 | BE822073 | Metabolism |
| 505 | GmaAffx.89772.8.S1_at | 0.17 | CK605778 | Metabolism |
| 506 | GmaAffx.91479.1.S1_s_at | 0.17 | CF807204 | No Homology to Known |
| | | | | Proteins |
| 507 | GmaAffx.89772.8.S1_x_at | 0.168 | CK605778 | Metabolism |
| 508 | Gma.7227.2.S1_a_at | 0.163 | AW759183 | Protein Destination & Storage |
| 509 | Gma.16477.1.S1_at | 0.159 | BE824134 | No Homology to Known |
| | | | | Proteins |
| 510 | Gma.13062.1.S1_at | 0.157 | CD394315 | Metabolism |
| 511 | Gma.12094.1.A1_at | 0.157 | CD395088 | Unclassified - Hypothetical |
| | | | | Protein Supported by cDNA |
| 512 | Gma.15093.1.S1_at | 0.153 | CD400374 | Protein Destination & Storage |
| 513 | Gma.8187.1.S1_a_at | 0.152 | BU549133 | Signal Transduction |
| 514 | Gma.4837.1.S1_at | 0.147 | BI969343 | Transporter |
| 515 | Gma.13296.3.S1_at | 0.147 | CD418313 | Transporter |
| 516 | Gma.10904.1.S1_at | 0.141 | CD408184 | Protein Destination & Storage |
| 517 | Gma.15534.1.S1_at | 0.133 | U50081.1 | Metabolism |
| 518 | GmaAffx.93212.1.S1_x_at | 0.129 | CF807524 | Transporter |
| 519 | GmaAffx.71222.1.S1_at | 0.124 | AW460050 | Metabolism |
| 520 | Gma.10830.1.S1_at | 0.123 | BE820822 | Unclassified - Hypothetical |
| | | | | Protein Supported by cDNA |
| 521 | Gma.12393.3.S1_at | 0.121 | BM085488 | Secondary Metabolism |
| 522 | GmaAffx.295.1.A1_s_at | 0.118 | BG653267 | Protein Destination & Storage |
| 523 | GmaAffx.75697.1.S1_s_at | 0.113 | BU090518 | Unclassified - Hypothetical |
| | | | | Protein Supported by cDNA |
| 524 | GmaAffx.1785.1.S1_at | 0.112 | BF325331 | Transcription |
| 525 | Gma.1527.2.S1_at | 0.104 | BE658017 | Secondary Metabolism |
| 526 | GmaAffx.24413.1.A1_at | 0.103 | BU548368 | Metabolism |
| 527 | Gma.1527.1.S1_a_at | 0.102 | BI968185 | Secondary Metabolism |
| 528 | Gma.176.1.S1_s_at | 0.101 | AF004810.1 | Protein Destination & Storage |
| 529 | GmaAffx.90812.1.S1_at | 0.1 | CF806537 | Disease & Defense |
| 530 | Gma.17985.1.S1_at | 0.099 | CD416450 | Metabolism |
| 531 | Gma.6921.1.S1_at | 0.0986 | BE823209 | Signal Transduction |
| 532 | Gma.3774.2.S1_at | 0.0892 | CD393365 | Energy |
| 533 | GmaAffx.73698.1.S1_at | 0.0875 | BU964780 | Metabolism |
| 534 | Gma.2986.1.S1_at | 0.0773 | BM271324 | Metabolism |
| 535 | GmaAffx.12933.1.S1_at | 0.0755 | AW395787 | Transcription |
| 536 | Gma.3000.1.S1_at | 0.0728 | BE657172 | No Homology to Known |
| | | | | Proteins |
| 537 | Gma.17326.1.A1_at | 0.0703 | AW568254 | Unclassified - Hypothetical |
| | | | | Protein NOT Supported by |
| | | | | cDNA |
| 538 | Gma.16566.1.S1_at | 0.0682 | CD400974 | Metabolism |
| 539 | Gma.3000.2.S1_a_at | 0.0673 | BM270844 | No Homology to Known |
| | | | | Proteins |
| 540 | Gma.9947.1.S1_at | 0.0631 | BF324269 | Unclassified - Hypothetical |
| | | | | Protein Supported by cDNA |
| 541 | GmaAffx.89407.1.A1_s_at | 0.0598 | CK606273 | Transporter |
| 542 | Gma.8318.1.A1_at | 0.0496 | BU550469 | No Homology to Known |
| | | | | Proteins |
| 543 | Gma.3774.1.S1_at | 0.0478 | BU550698 | Energy |
| 544 | GmaAffx.10204.1.S1_at | 0.0455 | BQ298213 | Unclassified - Hypothetical |
| | | | | Protein Supported by cDNA |
| 545 | Gma.3774.2.S1_a_at | 0.0366 | CD393365 | Energy |
| 546 | GmaAffx.68621.1.A1_at | 0.0276 | BI893889 | Unclassified - Protein with |
| | | | | Unknown Function |
| 547 | Gma.2912.1.S1_at | 0.0224 | AF117884.1 | Protein Destination & Storage |
| 548 | Gma.2912.1.S1_s_at | 0.0214 | AF117884.1 | Protein Destination & Storage |

| ID | Sub-Family | Gene Family | Additional Description |
|---|---|---|---|
| 1 | | | Predicted transporter (major |
| | | | facilitator superfamily) |
| 2 | Protein Kinase | | |
| 3 | | | |
| 4 | | | |
| 5 | | | |
| 6 | | | |
| 7 | Protein Kinase | | |
| 8 | | | Gelatinase A and related matrix |
| | | | metalloproteases |
| 9 | | | |
| 10 | Cytochrome | CYP93D | Cytochrome P450 CYP2 |
| | P450 | | subfamily |
| 11 | | | Cytochrome b5 |
| 11 | | | NADH-cytochrome b-5 reductase |
| 11 | | | Sulfite oxidase, molybdopterin- |
| | | | binding component |
| 12 | | | |
| 13 | | | |
| 14 | | | |
| 15 | | | |
| 16 | | | |
| 17 | | | |
| 18 | | | |
| 19 | Protein Kinase | | |
| 20 | Trehalose | | Trehalose-6-phosphate synthase |
| | Biosynthesis | | component TPS1 and related |
| | | | subunits |
| 21 | | | |
| 22 | Storage | | |
| | Protein | | |
| 23 | | | |
| 24 | | | Haloacid dehalogenase-like |
| | | | hydrolase |
| 25 | | | |
| 26 | | | |
| 27 | | | Aldehyde reductase |
| 28 | | | Aldehyde reductase |
| 29 | Trehalose | | Trehalose-6-phosphate synthase |
| | Biosynthesis | | component TPS1 and related |
| | | | subunits |
| 30 | | | |
| 31 | | | Gelatinase A and related matrix |
| | | | metalloproteases |
| 32 | | | Cytochrome b5 |
| 32 | | | NADH-cytochrome b-5 reductase |
| 32 | | | Sulfite oxidase, molybdopterin- |
| | | | binding component |
| 33 | | | Copper chaperone |
| 34 | | | Copper chaperone |
| 35 | | | Copper chaperone |
| 36 | | | |
| 37 | | | |
| 38 | | | |
| 39 | | | |
| 40 | | | |
| 41 | | | Branched chain aminotransferase |
| | | | BCAT1, pyridoxal phosphate |
| | | | enzymes type IV superfamily |
| 42 | | | |
| 43 | | | Thioredoxin |
| 44 | Protein Kinase | AtC1PKs | Serine/threonine protein kinase |
| 45 | | | Branched chain aminotransferase |
| | | | BCAT1, pyridoxal phosphate |
| | | | enzymes type IV superfamily |
| 46 | | | |
| 47 | | | |
| 48 | Phospholipid | | Glycosylphosphatidylinositol- |
| | Biosynthesis | | specific phospholipase C |
| 49 | | | Thioredoxin |
| 50 | Storage | | |
| | Protein | | |
| 51 | | | Aquaporin (major intrinsic protein |
| | | | family) |
| 52 | Transcription | Basic Leucine | |
| | Factor | Zipper (bZ1P) | |
| 53 | | | Copper chaperone |
| 54 | | | |
| 55 | Protein Kinase | | |
| 56 | | | |
| 57 | | | |
| 58 | Protein Kinase | AtC1PKs | Serine/threonine protein kinase |
| 59 | | | |
| 60 | | | |
| 61 | | | |
| 62 | Transcription | AP2/EREBP | |
| | Factor | | |
| 63 | | | Aquaporin (major intrinsic protein |
| | | | family) |
| 64 | | | |
| 65 | Protein Kinase | | |
| 66 | | | |
| 67 | Protein Kinase | | |
| 68 | | | |
| 69 | Calcium | | |
| | Binding | | |
| 70 | | | |
| 71 | | | |
| 72 | | | |
| 73 | | | |
| 74 | | | |
| 75 | Protein Kinase | | |
| 76 | | | Aquaporin (major intrinsic protein |
| | | | family) |
| 77 | | | |
| 78 | | | |
| 79 | | | Lipid phosphate phosphatase and |
| | | | related enzymes of the PAP2 |
| | | | family |
| 80 | Transcription | Zinc Finger | FOG: Zn-finger |
| | Factor | | |
| 81 | | | |
| 82 | Protein Kinase | | |
| 83 | | | |
| 84 | | | Predicted lipase |
| 85 | | | |
| 86 | | | |
| 87 | | | |
| 88 | Flavonoid | | |
| | Biosynthesis | | |
| 89 | | | |
| 90 | | | |
| 91 | | | |
| 92 | | | |
| 93 | | | |
| 94 | Protein Kinase | | Serine/threonine protein kinase |
| 95 | | | |
| 96 | | | |
| 97 | Protein Kinase | | Serine/threonine protein kinase |
| 98 | | | |
| 99 | | | |
| 100 | | | |
| 101 | | | |
| 102 | | | |
| 103 | Protein Kinase | | |
| 104 | | | Beta-glucosidase, lactase |
| | | | phlorizinhydrolase, and related |
| | | | proteins |
| 105 | | | |
| 106 | Transcription | AP2/EREBP | |
| | Factor | | |
| 107 | | | |
| 108 | | | |
| 109 | | | |
| 110 | | | |
| 111 | | | |
| 112 | Transcription | AP2/EREBP | |
| | Factor | | |
| 113 | | | |
| 114 | | | |
| 115 | | | Beta tubulin |
| 116 | | | |
| 117 | | | |
| 118 | Protein Kinase | | |
| 119 | | | Molecular chaperone (HSP90 |
| | | | family) |
| 120 | | | Serine carboxypeptidases |
| | | | (lysosomal cathepsin A) |
| 121 | Protein Kinase | | Serine/threonine protein kinase |
| 122 | | | Microtubule-associated protein |
| | | | essential for anaphase spindle |
| | | | elongation |
| 123 | | | |
| 124 | | | |
| 125 | | | |
| 126 | | | |
| 127 | | | |
| 128 | Hormone | | Voltage-gated shaker-like K+ |
| | Induced | | channel, subunit beta/KCNAB |
| 129 | | | |
| 130 | | | |
| 131 | Protein Kinase | | Serine/threonine protein kinase |
| 132 | | | |
| 133 | | | |
| 134 | | | Hydroxyindole-O- |
| | | | methyltransferase and related |
| | | | SAM-dependent |
| | | | methyltransferases |
| 135 | | | |
| 136 | | | |
| 137 | | | |
| 138 | Protein Kinase | AtCIPKs | Serine/threonine protein kinase |
| 139 | Transcription | Zinc Finger | GATA-4/5/6 transcription factors |
| | Factor | | |
| 140 | | | Branched chain aminotransferase |
| | | | BCAT1, pyridoxal phosphate |
| | | | enzymes type IV superfamily |
| 141 | | | |
| 142 | | | |
| 143 | | | |
| 144 | | | |
| 145 | | | |
| 146 | | | |
| 147 | | | |
| 148 | | | |
| 149 | Flavonoid | | Flavonol reductase/cinnamoyl- |
| | Biosynthesis | | CoA reductase |
| 150 | | | |
| 151 | Calcium | | Calmodulin and related proteins |
| | Binding | | (EF-Hand superfamily) |
| 152 | | | |
| 153 | | | |
| 154 | | | |
| 155 | | | Ferric reductase, NADH/NADPH |
| | | | oxidase and related proteins |
| 156 | | | |
| 157 | | | |
| 158 | MAPKK | | Mitogen-activated protein kinase |
| | | | kinase (MAP2K) |
| 159 | | | |
| 160 | | | Beta-glucosidase, lactase |
| | | | phlorizinhydrolase, and related |
| | | | proteins |
| 161 | | | Hydroxyindole-O- |
| | | | methyltransferase and related |
| | | | SAM-dependent |
| | | | methyltransferases |
| 162 | Transcription | MYB | Transcription factor, Myb |
| | Factor | | superfamily |
| 163 | Storage | | |
| | Protein | | |
| 164 | | | |
| 165 | | | |
| 166 | | | |
| 167 | | | |
| 168 | | | |
| 169 | Auxin | | |
| | Transport | | |
| 170 | | | |
| 171 | | | |
| 172 | Protein | | Serine/threonine protein |
| | Phosphatase | | phosphatase |
| 173 | | | Predicted E3 ubiquitin ligase |
| 174 | | | Leucine rich repeat proteins, some |
| | | | proteins contain F-box |
| 175 | Transcription | Unclassified | |
| | Factor | | |
| 176 | Protein Kinase | | FOG: Transposon-encoded |
| | | | proteins with TYA, reverse |
| | | | transcriptase, integrase domains in |
| | | | various combinations |
| 177 | | | FOG: PPR repeat |
| 178 | | | |
| 179 | | | |
| 180 | | | |
| 181 | | | Alanine-glyoxylate |
| | | | aminotransferase AGT2 |
| 182 | | | |
| 183 | | | AAA+-type ATPase |
| 184 | | | |
| 185 | Ethylene | | Iron/ascorbate family |
| | Biosynthesis | | oxidoreductases |
| 186 | Transcription | Unclassified | GATA-4/5/6 transcription factors |
| | Factor | | |
| 187 | | | |
| 188 | | | |
| 189 | Transcription | Homeodomain | Transcription factor MEIS1 and |
| | Factor | | related HOX domain proteins |
| 190 | | | |
| 191 | | | |
| 192 | | | |
| 193 | Transcription | Basic Helix- | |
| | Factor | Loop-Helix | |
| | | (bHLH) | |
| 194 | | | |
| 195 | Transcription | Homeodomain | Transcription factor MEIS1 and |
| | Factor | | related HOX domain proteins |
| 196 | | | |
| 197 | GA | | Iron/ascorbate family |
| | Biosynthesis | | oxidoreductases |
| 198 | | | |
| 199 | | | |
| 200 | | | Phosphoinositide-specific |
| | | | phospholipase C |
| 201 | | | Metacaspase involved in |
| | | | regulation of apoptosis |
| 202 | | | Sulfotransferase |
| 203 | | | |
| 204 | | | |
| 205 | | | Sulfate/bicarbonate/oxalate |
| | | | exchanger SAT-1 and related |
| | | | transporters (SLC26 family) |
| 206 | | | |
| 207 | | | |
| 208 | | | |
| 209 | | | |
| 210 | | | |
| 211 | Transcription | Zinc Finger | FOG: Zn-finger |
| | Factor | | |
| 212 | | | |
| 213 | | | |
| 214 | | | Translation initiation factor 5A |
| | | | (eIF-5A) |
| 215 | Transcription | MYB | Transcription factor, Myb |
| | Factor | | superfamily |
| 216 | | | |
| 217 | Transcription | NAC Domain | |
| | Factor | | |
| 218 | | | Predicted E3 ubiquitin ligase |
| 219 | | | |
| 220 | | | |
| 221 | Transcription | Unclassified | Zuotin and related molecular |
| | Factor | | chaperones (DnaJ superfamily), |
| | | | contains DNA-binding domains |
| 222 | MAPKKK | MEKK | MEKK and related |
| | | | serine/threonine protein kinases |
| 223 | | | Predicted dehydrogenase |
| 224 | | | Purple (tartrate-resistant) acid |
| | | | phosphatase |
| 225 | Transcription | MYB | Transcription factor, Myb |
| | Factor | | superfamily |
| 226 | | | |
| 227 | | | Ubiquitin-protein ligase |
| 228 | | | Leucine rich repeat proteins, some |
| | | | proteins contain F-box |
| 229 | | | |
| 230 | | | |
| 231 | | | UDP-glucuronosyl and UDP- |
| | | | glucosyl transferase |
| 232 | Transcription | AP2/EREBP | |
| | Factor | | |
| 233 | | | |
| 234 | | | Chitinase |
| 235 | | | |
| 236 | | | FOG: Predicted E3 ubiquitin |
| | | | ligase |
| 237 | | | |
| 238 | | | GTPase Rab18, small G protein |
| | | | superfamily |
| 239 | | | |
| 240 | | | Glutathione S-transferase |
| 241 | | | |
| 242 | | | UDP-galactose transporter related |
| | | | protein |
| 243 | | | Acyl-CoA synthetase |
| 244 | Transcription | NAC Domain | |
| | Factor | | |
| 245 | | | |
| 246 | Unclassified | | |
| 247 | | | |
| 248 | | | O-methyltransferase |
| 249 | | | |
| 250 | | | |
| 251 | | | Protein involved in thiamine |
| | | | biosynthesis and DNA damage |
| | | | tolerance |
| 252 | | | |
| 253 | Transcription | Basic Helix- | |
| | Factor | Loop-Helix | |
| | | (bHLH) | |
| 254 | Protein | | Serine/threonine protein |
| | Phosphatase | | phosphatase |
| 255 | | | |
| 256 | | | |
| 257 | | | |
| 258 | Transcription | WRKY | |
| | Factor | | |
| 259 | | | Uncharacterized conserved protein |
| 260 | | | |
| 261 | Phytochrome | | eIF-2alpha kinase PEK/EIF2AK3 |
| 262 | Transcription | Homeodomain | Transcription factor HEX, |
| | Factor | | contains HOX and HALZ domains |
| 263 | | | |
| 264 | | | Transketolase |
| 265 | | | |
| 266 | | | Galactosyltransferases |
| 267 | | | |
| 268 | | | |
| 269 | | | Flavin-containing monooxygenase |
| 270 | | | |
| 271 | | | |
| 272 | | | |
| 273 | | | Predicted transporter (major |
| | | | facilitator superfamily) |
| 274 | Ethylene | | Iron/ascorbate family |
| | Biosynthesis | | oxidoreductases |
| 275 | Protein | | Serine/threonine protein |
| | Phosphatase | | phosphatase |
| 276 | | | Aspartyl protease |
| 277 | | | Phosphatidylethanolamine binding |
| | | | protein |
| 278 | Ethylene | | Iron/ascorbate family |
| | Biosynthesis | | oxidoreductases |
| 279 | | | |
| 280 | | | |
| 281 | | | Aquaporin (major intrinsic protein |
| | | | family) |
| 282 | | | |
| 283 | | | Zn-finger protein |
| 284 | | | NADH pyrophosphatase 1 of the |
| | | | Nndix family of hydrolases |
| 285 | Ethylene | | Iron/ascorbate family |
| | Biosynthesis | | oxidoreductases |
| 286 | | | |
| 287 | | | |
| 288 | | | |
| 289 | Transcription | MYB | Transcription factor, Myb |
| | Factor | | snperfamily |
| 290 | Calcium | Calcinm | |
| | Binding | Dependent | |
| | | Protein Kinase | |
| 291 | | | |
| 292 | | | |
| 293 | | | |
| 294 | | | |
| 295 | | | |
| 296 | | | |
| 297 | | | |
| 298 | | | |
| 299 | | | |
| 300 | | | |
| | | | Alpha-L-fucosidase |
| 301 | | | |
| 302 | Transcription | Unclassified | CCAAT-binding factor, subunit B |
| | Factor | | (HAP2) |
| 303 | | | Uncharacterized conserved protein |
| 304 | Transcription | Zinc Finger | |
| | Factor | | |
| 305 | | | Flavonol reductase/cinnamoyl- |
| | | | CoA reductase |
| 306 | | | Acyl-CoA reductase |
| 307 | | | Ubiquitin fusion-degradation |
| | | | protein |
| 308 | Unclassified | | Lanthionine synthetase C-like |
| | | | protein 1 |
| 309 | | | Zn-finger protein |
| 310 | | | |
| 311 | | | |
| 312 | | | |
| 313 | | | Hydroxyindole-O- |
| | | | methyltransferase and related |
| | | | SAM-dependent |
| | | | methyltransferases |
| 314 | Ethylene | | Iron/ascorbate family |
| | Biosynthesis | | oxidoreductases |
| 315 | | | |
| 316 | | | |
| 317 | | | |
| 318 | | | |
| 319 | | | |
| 320 | | | |
| 321 | | | |
| 322 | | | |
| 323 | | | Protein involved in thiamine |
| | | | biosynthesis and DNA damage |
| | | | tolerance |
| 324 | Ubiquitin | | FOG: Predicted E3 ubiquitin |
| | Degradation | | ligase |
| 325 | | | Glutathione peroxidase |
| 326 | Protein Kinase | | |
| 327 | | | Iron/ascorbate family |
| | | | oxidoreductases |
| 328 | | | Serine carboxypeptidases |
| 329 | | | |
| 330 | | | |
| 331 | | | Glutamate decarboxylase and |
| | | | related proteins |
| 332 | | | |
| 333 | | | Acyl-CoA synthetase |
| 334 | Transcription | NAC Domain | |
| | Factor | | |
| 335 | | | |
| 336 | | | |
| 337 | | | |
| 338 | | | Long-chain acyl-CoA synthetases |
| | | | (AMP-forming) |
| 339 | | | GPI-alpha-mannosyltransferase III |
| | | | (GP110/PIG-B) involved in |
| | | | glycosylphosphatidylinositol |
| | | | anchor biosynthesis |
| 340 | | | Dehydrogenase kinase |
| 341 | | | |
| 342 | | | |
| 343 | Cytochrome | CYP716A | Cytochrome P450 |
| | P450 | | CYP4/CYP19/CYP26 subfamilies |
| 344 | | | |
| 345 | | | |
| 346 | | | |
| 347 | | | Amino acid transporters |
| 348 | | | |
| 349 | | | Beta-galactosidase |
| 350 | Protein Kinase | | Putative serine/threonine protein |
| | | | kinase |
| 351 | | | |
| 352 | | | |
| 353 | | | |
| 354 | | | |
| 355 | | | |
| 356 | | | Ubiquitin-protein ligase |
| 357 | | | |
| 358 | | | Chitinase |
| 359 | Transcription | Basic Helix- | |
| | Factor | Loop-Helix | |
| | | (bHLH) | |
| 360 | | | |
| 361 | | | |
| 362 | | | FKBP-type peptidyl-prolyl cis- |
| | | | trans isomerase |
| 363 | | | Haloacid dehalogenase-like |
| | | | hydrolase |
| 364 | | | Ferric reductase, NADH/NADPH |
| | | | oxidase and related proteins |
| 365 | | | |
| 366 | Ethylene | | Iron/ascorbate family |
| | Biosynthesis | | oxidoreductases |
| 367 | | | |
| 368 | | | |
| 369 | Transcription | Heat Shock | Heat shock transcription factor |
| | Factor | | |
| 370 | | | |
| 371 | | | |
| 372 | | | |
| 373 | | | |
| 374 | | | |
| 375 | | | |
| 376 | Ethylene | | Iron/ascorbate family |
| | Biosynthesis | | oxidoreductases |
| 377 | | | Hydroxyindole-O- |
| | | | methyltransferase and related |
| | | | SAM-dependent |
| | | | methyltransferases |
| 378 | Protein Kinase | | |
| 379 | | | |
| 380 | | | |
| 381 | | | |
| 382 | | | O-methyltransferase |
| 383 | | | Serine carboxypeptidases |
| 384 | Transcription | Basic Helix- | |
| | Factor | Loop-Helix | |
| | | (bHLH) | |
| 385 | | | |
| 386 | | | |
| 387 | | | |
| 388 | Hormone | | |
| | Induced | | |
| 389 | Transcription | Basic Helix- | |
| | Factor | Loop-Helix | |
| | | (bHLH) | |
| 390 | Transcription | NAC Domain | |
| | Factor | | |
| 391 | | | Homocysteine S-methyltransferase |
| 392 | | | Reductases with broad range of |
| | | | substrate specificities |
| 393 | | | Ca2+/H+ antiporter VCX1 and |
| | | | related proteins |
| 394 | Transcription | Homeodomain | Transcription factor MEIS1 and |
| | Factor | | related HOX domain proteins |
| 395 | Transcription | Heat Shock | Heat shock transcription factor |
| | Factor | | |
| 396 | | | |
| 397 | | | |
| 398 | | | Glutaredoxin-related protein |
| 399 | | | Permease of the major facilitator |
| | | | superfamily |
| 400 | | | NADH:flavin oxidoreductase/12- |
| | | | oxophytodienoate reductase |
| 401 | | | |
| 402 | Transcription | MYB | |
| | Factor | | |
| 403 | | | Iron/ascorbate family |
| | | | oxidoreductases |
| 404 | | | Serine carboxypeptidases |
| 405 | | | |
| 406 | | | Glycine cleavage system H protein |
| | | | (lipoate-binding) |
| 407 | | | H+/oligopeptide symporter |
| 408 | | | Gamma-glutamyl hydrolase |
| 409 | | | |
| 410 | | | |
| 411 | | | |
| 412 | | | |
| 413 | Transcription | Basic Leucine | |
| | Factor | Zipper (bZIP) | |
| 414 | Storage | | Reticulon |
| | Protein | | |
| 415 | | | Soluble epoxide hydrolase |
| 416 | | | |
| 417 | | | |
| 418 | Unclassified | | Lanthionine synthetase C-like |
| | | | protein 1 |
| 419 | | | |
| 420 | | | |
| 421 | | | Molecular chaperones |
| | | | HSP70/HSC70, HSP70 |
| | | | superfamily |
| 422 | | | |
| 423 | | | |
| 424 | | | |
| 425 | | | Glyoxylate/hydroxypyruvate |
| | | | reductase (D-isomer-specific 2- |
| | | | hydroxy acid dehydrogenase |
| | | | superfamily) |
| 426 | Protein | | |
| | Phosphatase | | |
| 427 | Transcription | MYB | Transcription factor, Myb |
| | Factor | | superfamily |
| 428 | | | Haloacid dehalogenase-like |
| | | | hydrolase |
| 429 | Protein | | Serine/threonine protein |
| | Phosphatase | | phosphatase |
| 430 | | | |
| 431 | Storage | | |
| | Protein | | |
| 432 | Transcription | AP2/EREBP | |
| | Factor | | |
| 433 | | | Predicted metallothionein |
| 434 | Protein | | Serine/threonine protein |
| | Phosphatase | | phosphatase |
| 435 | | | |
| 436 | | | |
| 437 | | | |
| 438 | | | Uncharacterized conserved protein |
| 439 | | | |
| 440 | | | Transporter, ABC superfamily |
| | | | (Breast cancer resistance protein) |
| 441 | | | |
| 442 | Transcription | Basic Helix- | |
| | Factor | Loop-Helix | |
| | | (bHLH) | |
| 443 | | | |
| 444 | Transcription | Basic Leucine | |
| | Factor | Zipper (bZIP) | |
| 445 | | | |
| 446 | | | |
| 447 | | | |
| 448 | | | FOG: Predicted E3 ubiquitin |
| | | | ligase |
| 449 | | | |
| 450 | Transcription | Basic Helix- | |
| | Factor | Loop-Helix | |
| | | (bHLH) | |
| 451 | | | |
| 452 | | | Reductases with broad range of |
| | | | substrate specificities |
| 453 | | | Aquaporin (major intrinsic protein |
| | | | family) |
| 454 | | | |
| 455 | Amino Acid | | Arginase family protein |
| | Biosynthesis/ | | |
| | Metabolism | | |
| 456 | | | |
| 457 | | | Flavin-containing monooxygenase |
| 458 | | | Aldehyde dehydrogenase |
| 459 | | | Asparaginyl peptidases |
| 460 | | | Beta, beta-carotene 15,15′- |
| | | | dioxygenase and related enzymes |
| 461 | | | |
| 462 | | | Alcohol dehydrogenase, class V |
| 463 | | | |
| 464 | | | |
| 465 | | | Endoplasmic reticulum protein |
| | | | EP58, contains filamin rod domain |
| | | | and KDEL motif |
| 466 | | | |
| 467 | | | |
| 468 | | | |
| 469 | | | Aquaporin (major intrinsic protein |
| | | | family) |
| 470 | | | |
| 471 | Transcription | NAC Domain | |
| | Factor | | |
| 472 | | | Selenium-binding protein |
| 473 | Protein | | Serine/threonine protein |
| | Phosphatase | | phosphatase |
| 474 | Transcription | CCAAT | CCAAT-binding factor, subunit A |
| | Factor | | (HAP3) |
| 475 | | | |
| 476 | | | Transporter, ABC superfamily |
| | | | (Breast cancer resistance protein) |
| 477 | | | |
| 478 | | | |
| 479 | Transcription | Zinc Finger | |
| | Factor | | |
| 480 | | | |
| 481 | | | Flavonol reductase/cinnamoyl- |
| | | | CoA reductase |
| 482 | Transcription | NAC Domain | |
| | Factor | | |
| 483 | | | |
| 484 | Transcription | Basic Helix- | |
| | Factor | Loop-Helix | |
| | | (bHLH) | |
| 485 | | | Kynurenine 3-monooxygenase and |
| | | | related flavoprotein |
| | | | monooxygenases |
| 486 | | | |
| 487 | | | |
| 488 | | | |
| 489 | | | |
| 490 | | | Phenylalanine and histidine |
| | | | ammonia-lyase |
| 491 | | | |
| 492 | Storage | | Uncharacterized conserved protein |
| | Protein | | |
| 493 | | | Tomosyn and related SNARE- |
| | | | interacting proteins |
| 494 | | | Predicted haloacid dehalogenase- |
| | | | like hydrolase |
| 495 | Protein | | |
| | Phosphatase | | |
| 496 | | | |
| 497 | Protein | | |
| | Phosphatase | | |
| 498 | | | |
| 499 | | | |
| 500 | | | |
| 501 | | | |
| 502 | | | Tomosyn and related SNARE- |
| | | | interacting proteins |
| 503 | | | |
| 504 | | | |
| 505 | | | |
| 506 | | | |
| 507 | | | |
| 508 | | | Ubiquitin-protein ligase |
| 509 | | | |
| 510 | | | |
| 511 | | | |
| 512 | | | Glutathione S-transferase |
| 513 | Protein | | Serine/threonine protein |
| | Phosphatase | | phosphatase |
| 514 | | | H+/oligopeptide symporter |
| 515 | | | |
| 516 | | | |
| 517 | | | |
| 518 | | | |
| 519 | | | 5′-AMP-activated protein kinase, |
| | | | gamma subunit |
| 520 | | | |
| 521 | | | Beta, beta-carotene 15,15′- |
| | | | dioxygenase and related enzymes |
| 522 | | | |
| 523 | | | |
| 524 | Transcription | Homeodomain | Transcription factor HEX, |
| | Factor | | contains HOX and HALZ domains |
| 525 | | | Flavonol reductase/cinnamoyl- |
| | | | CoA reductase |
| 526 | | | 5′-AMP-activated protein kinase, |
| | | | gamma subunit |
| 527 | | | Flavonol reductase/cinnamoyl- |
| | | | CoA reductase |
| 528 | Storage | | |
| | Protein | | |
| 529 | | | |
| 530 | | | |
| 531 | Unclassified | | Phosphatidylethanolamine binding |
| | | | protein |
| 532 | | | Thioredoxin, nucleoredoxin and |
| | | | related proteins |
| 533 | | | 5′-AMP-activated protein kinase, |
| | | | gamma subunit |
| 534 | | | 5′-AMP-activated protein kinase, |
| | | | gamma subunit |
| 535 | Transcription | CCAAT | CCAAT-binding factor, subunit B |
| | Factor | | (HAP2) |
| 536 | | | |
| 537 | | | Permease of the major facilitator |
| | | | superfamily |
| 538 | | | Fumarase |
| 539 | | | |
| 540 | | | |
| 541 | | | |
| 542 | | | |
| 543 | | | Thioredoxin, nucleoredoxin and |
| | | | related proteins |
| 544 | | | |
| 545 | | | Thioredoxin, nucleoredoxin and |
| | | | related proteins |
| 546 | Stress Induced | | |
| 547 | Storage | | Uncharacterized conserved protein |
| | Protein | | |
| 548 | Storage | | Uncharacterized conserved protein |
| | Protein | | |

TABLE 2

| ID | Gene Name | Fold Change | Soybean_Gen Bank_Acc | Functional Category | Sub-Family | Gene Family | Additional Description |
|---|---|---|---|---|---|---|---|
| 1 | Gma.4058.1.S1_at | 22.22 | AW310338 | Transporter | | | Predicted transporter (major |
| | | | | | | | facilitator superfamily) |
| 2 | GmaAffx.32345.1.A1_s_at | 16.29 | BI970277 | Cell Structure | | | |
| 3 | Gma.5716.1.S1_at | 11.64 | BQ453925 | No Homology to Known Proteins | | | |
| 4 | GmaAffx.87679.1.S1_at | 9.533 | BM523734 | Metabolism | Trehalose | | Trehalose-6-phosphate |
| | | | | | Biosynthesis | | synthase component TPS1 |
| | | | | | | | and related subunits |
| 5 | GmaAffx.89567.1.A1_at | 9.001 | CK606157 | Signal Transduction | Calcium Binding | Calcium | Ca2+/calmodulin-dependent |
| | | | | | | Dependent | protein kinase, EF-Hand |
| | | | | | | Protein Kinase | protein superfamily |
| 6 | GmaAffx.19116.1.S1_at | 7.985 | BI968419 | Signal Transduction | Protein Kinase | | |
| 7 | GmaAffx.87679.2.S1_s_at | 7.575 | AW102083 | Metabolism | Trehalose | | Trehalose-6-phosphate |
| | | | | | Biosynthesis | | synthase component TPS1 |
| | | | | | | | and related subunits |
| 8 | Gma.3804.1.A1_at | 7.423 | AW310249 | Disease & Defense | | | |
| 9 | GmaAffx.9866.1.S1_at | 7.144 | BM524432 | Metabolism | | | |
| 10 | GmaAffx.9618.1.S1_at | 7.08 | BM092432 | No Homology to Known Proteins | | | |
| 11 | Gma.8416.1.S1_at | 6.333 | U13987.1 | Metabolism | | | Cytochrome b5 |
| 11 | Gma.8416.1.S1_at | 6.333 | U13987.1 | Metabolism | | | NADH-cytochrome b-5 reductase |
| 11 | Gma.8416.1.S1_at | 6.333 | U13987.1 | Metabolism | | | Sulfite oxidase, |
| | | | | | | | molybdopterin-binding component |
| 12 | GmaAffx.89396.2.A1_s_at | 6.061 | CK605844 | Cell Structure | | | |
| 13 | GmaAffx.70875.1.S1_s_at | 6.049 | AW100693 | No Homology to Known Proteins | | | |
| 14 | GmaAffx.40860.2.S1_at | 5.75 | BG509282 | Disease & Defense | | | |
| 15 | Gma.10921.1.S1_s_at | 5.722 | BQ611250 | No Homology to Known Proteins | | | |
| 16 | Gma.152.1.S1_at | 5.694 | Y10492.1 | Metabolism | Cytochrome P450 | CYP93D | Cytochrome P450 CYP2 subfamily |
| 17 | Gma.17961.1.S1_at | 5.671 | AW350363 | Protein Destination & | Storage Protein | | |
| | | | | Storage | | | |
| 18 | GmaAffx.935412.S1_at | 5.572 | CF805889 | Protein Destination & | | | Gelatinase A and related |
| | | | | Storage | | | matrix metalloproteases |
| 19 | Gma.16425.2.S1_s_at | 5.528 | AW099501 | Unclassified - | | | Copper chaperone |
| | | | | Hypothetical Protein | | | |
| | | | | NOT Supported by cDNA | | | |
| 20 | Gma.11259.1.S1_at | 5.423 | AI442316 | Unclassified - | | | |
| | | | | Hypothetical Protein | | | |
| | | | | Supported by cDNA | | | |
| 21 | Gma.3888.2.S1_at | 5.349 | BM139532 | Unclassified - | | | Aldehyde reductase |
| | | | | Hypothetical Protein | | | |
| | | | | NOT Supported by cDNA | | | |
| 22 | GmaAffx.2743.1.S1_s_at | 5.137 | BM568087 | Signal Transduction | Protein Kinase | | |
| 23 | Gma.5629.1.S1_at | 5.076 | BQ740550 | Disease & Defense | | | |
| 24 | GmaAffx.81930.1.S1_at | 5.036 | BG789998 | Unclassified - | | | |
| | | | | Hypothetical Protein | | | |
| | | | | Supported by cDNA | | | |
| 25 | GmaAffx.2743.1.S1_at | 5.014 | BM568087 | Signal Transduction | Protein Kinase | | |
| 26 | Gma.5483.1.S1_s_at | 4.957 | CD414581 | Transcription | Transcription | Basic Leucine | |
| | | | | | Factor | Zipper (bZIP) | |
| 27 | Gma.4701.2.A1_a_at | 4.942 | BM526048 | Energy | | | Thioredoxin |
| 28 | Gma.4226.2.S1_at | 4.938 | BM568170 | Unclassified - | | | |
| | | | | Hypothetical Protein | | | |
| | | | | Supported by cDNA | | | |
| 29 | Gma.3639.1.S1_at | 4.902 | AB083030.1 | Protein Destination & | Storage Protein | | |
| | | | | Storage | | | |
| 30 | GmaAffx.70509.1.A1_at | 4.887 | CD400633 | No Homology to Known Proteins | | | |
| 31 | Gma.1400.2.S1_at | 4.811 | BG510739 | Signal Transduction | Protein Kinase | AtCIPKs | Serine/threonine protein kinase |
| 32 | GmaAffx.93260.1.S1_at | 4.807 | CF808985 | Signal Transduction | Protein Kinase | | |
| 33 | Gma.4071.1.S1_at | 4.771 | BG651692 | Unclassified - | | | |
| | | | | Hypothetical Protein | | | |
| | | | | Supported by cDNA | | | |
| 34 | GmaAffx.69888.1.A1_at | 4.769 | BU549275 | No Homology to Known Proteins | | | |
| 35 | Gma.3579.1.S1_at | 4.768 | AW349799 | Unclassified - | | | Kelch repeat-containing proteins |
| | | | | Hypothetical Protein | | | |
| | | | | Supported by cDNA | | | |
| 36 | GmaAffx.87872.1.S1_at | 4.606 | BM885036 | Metabolism | | | CTP synthase (UTP-ammonia lyase) |
| 37 | GmaAffx.18846.1.S1_at | 4.583 | BM091991 | Signal Transduction | Small GTPase | | FOG: WD40 repeat |
| 38 | GmaAffx.89075.1.S1_s_at | 4.503 | CK606454 | No Homology to Known Proteins | | | |
| 39 | GmaAffx.43297.1.A1_at | 4.485 | AI442864 | Unclassified - | | | |
| | | | | Hypothetical Protein | | | |
| | | | | NOT Supported by cDNA | | | |
| 40 | Gma.17605.1.S1_at | 4.441 | BI968208 | Secondary Metabolism | Flavonoid | | |
| | | | | | Biosynthesis | | |
| 41 | GmaAffx.89158.1.A1_at | 4.344 | CK605958 | Intracellular Traffic | | | Phosphatidylinositol transfer |
| | | | | | | | protein SEC14 and related proteins |
| 42 | GmaAffx.93462.1.S1_s_at | 4.271 | CF809187 | Signal Transduction | Protein Kinase | AtCIPKs | Serine/threonine protein kinase |
| 43 | Gma.16425.3.S1_s_at | 4.229 | AW508123 | Unclassified - Protein | | | Copper chaperone |
| | | | | with Unknown Function | | | |
| 44 | Gma.7474.1.A1_at | 4.206 | BU547968 | Unclassified - | | | |
| | | | | Hypothetical Protein | | | |
| | | | | Supported by cDNA | | | |
| 45 | Gma.16832.1.S1_s_at | 4.194 | L22029.1 | No Homology to Known | | | |
| | | | | Proteins | | | |
| 46 | GmaAffx.52494.1.S1_at | 4.174 | BE473800 | Unclassified - | | | |
| | | | | Hypothetical Protein | | | |
| | | | | NOT Supported by | | | |
| | | | | cDNA | | | |
| 47 | Gma.5689.3.S1_s_at | 4.168 | BE657645 | Protein Destination & | | | Gelatinase A and related |
| | | | | Storage | | | matrix metalloproteases |
| 48 | GmaAffx.62542.1.S1_at | 4.057 | BE659136 | Unclassified - | | | |
| | | | | Hypothetical Protein | | | |
| | | | | NOT Supported by | | | |
| | | | | cDNA | | | |
| 49 | GmaAffx.48172.1.S1_at | 4.033 | BF009291 | Transporter | | | Predicted small molecule |
| | | | | | | | transporter |
| 50 | GmaAffx.92537.1.S1_at | 4.019 | CF808262 | Energy | | | Thioredoxin |
| 51 | Gma.184.1.S1_at | 3.978 | AW101261 | Unclassified - | | | |
| | | | | Hypothetical Protein | | | |
| | | | | Supported by cDNA | | | |
| 52 | Gma.7726.1.A1_at | 3.929 | BM886413 | Unclassified - Protein | | | Ca2+-binding protein |
| | | | | with Unknown Function | | | (centrin/caltractin), EF-Hand |
| | | | | | | | superfamily protein |
| 53 | Gma.15889.1.S1_at | 3.928 | AW348133 | Transcription | Transcription | WRKY | |
| | | | | | Factor | | |
| 54 | Gma.2010.1.S1_a_at | 3.876 | CD402050 | Transcription | Transcription | Zinc Finger | GATA-4/5/6 transcription |
| | | | | | Factor | | factors |
| 55 | GmaAffx.55767.1.S1_at | 3.817 | BQ079515 | Signal Transduction | Calcium Binding | | Calmodulin and related |
| | | | | | | | proteins (EF-Hand |
| | | | | | | | superfamily) |
| 56 | GmaAffx.78720.2.S1_at | 3.816 | BI788325 | No Homology to Known | | | |
| | | | | Proteins | | | |
| 57 | Gma.6555.1.S1_at | 3.81 | BM954521 | Unclassified - | | | |
| | | | | Hypothetical Protein | | | |
| | | | | Supported by cDNA | | | |
| 58 | PsAffx.psHB044xM02f_at | 3.793 | | | | | |
| 59 | Gma.5631.1.S1_at | 3.748 | CD405214 | Transporter | | | Amino acid transporters |
| 60 | GmaAffx.24583.1.S1_at | 3.746 | BE609540 | Signal Transduction | Protein Kinase | | Serine/threonine protein |
| | | | | | | | kinase |
| 61 | Gma.2446.2.S1_at | 3.739 | BI427101 | Unclassified - | | | |
| | | | | Hypothetical Protein | | | |
| | | | | Supported by cDNA | | | |
| 62 | Gma.5816.1.S1_at | 3.726 | BI787790 | Unclassified - | | | Aldehyde reductase |
| | | | | Hypothetical Protein | | | |
| | | | | Supported by cDNA | | | |
| 63 | GmaAffx.93270.1.S1_x_at | 3.706 | CF808995 | No Homology to Known | | | |
| | | | | Proteins | | | |
| 64 | GmaAffx.78636.1.S1_at | 3.706 | BQ627652 | No Homology to Known | | | |
| | | | | Proteins | | | |
| 65 | GmaAffx.18649.1.S1_at | 3.693 | AW351025 | No Homology to Known | | | |
| | | | | Proteins | | | |
| 66 | GmaAffx.10340.1.S1_x_at | 3.668 | CA938421 | No Homology to Known | | | |
| | | | | Proteins | | | |
| 67 | Gma.13447.1.A1_at | 3.656 | CD398305 | No Homology to Known | | | |
| | | | | Proteins | | | |
| 68 | GmaAffx.9145.1.S1_s_at | 3.649 | BU763814 | Disease & Defense | | | |
| 69 | GmaAffx.65829.1.A1_at | 3.588 | CD392418 | Transcription | Transcription | AP2/EREBP | |
| | | | | | Factor | | |
| 70 | Gma.17789.1.S1_at | 3.562 | BG511704 | Unclassified - | | | |
| | | | | Hypothetical Protein | | | |
| | | | | Supported by cDNA | | | |
| 71 | Gma.12665.2.A1_at | 3.529 | CD394142 | Unclassified - | | | |
| | | | | Hypothetical Protein | | | |
| | | | | Supported by cDNA | | | |
| 72 | Gma.14471.2.S1_at | 3.523 | CD408292 | Transporter | | | Aquaporin (major intrinsic |
| | | | | | | | protein family) |
| 73 | Gma.15889.1.S1_s_at | 3.503 | AW348133 | Transcription | Transcription | WRKY | |
| | | | | | Factor | | |
| 74 | Gma.2488.1.S1_at | 3.467 | CD406079 | Protein Destination & | | | Serine carboxypeptidases |
| | | | | Storage | | | (lysosomal cathepsin A) |
| 75 | Gma.5579.1.S1_at | 3.467 | AW119360 | Secondary Metabolism | Flavonoid | | |
| | | | | | Biosynthesis | | |
| 76 | GmaAffx.36514.1.S1_at | 3.452 | BE658341 | Disease & Defense | | | |
| 77 | GmaAffx.8098.1.S1_at | 3.437 | AW156410 | No Homology to Known | | | |
| | | | | Proteins | | | |
| 78 | GmaAffx.89512.1.A1_at | 3.403 | CK606530 | No Homology to Known | | | |
| | | | | Proteins | | | |
| 79 | GmaAffx.47815.1.A1_at | 3.374 | CD415065 | Unclassified - | | | |
| | | | | Hypothetical Protein | | | |
| | | | | NOT Supported by | | | |
| | | | | cDNA | | | |
| 80 | Gma.8896.1.S1_at | 3.369 | BQ252812 | Transcription | Transcription | AP2/EREBP | |
| | | | | | Factor | | |
| 81 | GmaAffx.18566.1.A1_at | 3.363 | BU550417 | Unclassified - | | | |
| | | | | Hypothetical Protein | | | |
| | | | | NOT Supported by | | | |
| | | | | cDNA | | | |
| 82 | Gma.1221.1.S1_s_at | 3.359 | L23854.1 | Metabolism | | | Cytochrome b5 |
| 82 | Gma.1221.1.S1_s_at | 3.359 | L23854.1 | Metabolism | | | NADH-cytochrome b-5 |
| | | | | | | | reductase |
| 82 | Gma.1221.1.S1_s_at | 3.359 | L23854.1 | Metabolism | | | Sulfite oxidase, |
| | | | | | | | molybdopterin-binding |
| | | | | | | | component |
| 83 | GmaAffx.90960.1.A1_s_at | 3.356 | CF806685 | Signal Transduction | Protein Kinase | | |
| 84 | Gma.12665.1.S1_at | 3.331 | BE347113 | Unclassified - | | | |
| | | | | Hypothetical Protein | | | |
| | | | | Supported by cDNA | | | |
| 85 | Gma.340.1.S1_at | 3.327 | AW350127 | Cell Structure | | | |
| 86 | Gma.6266.1.A1_at | 3.296 | CD413418 | Unclassified - Protein | | | |
| | | | | with Unknown Function | | | |
| 87 | GmaAffx.85374.1.S1_at | 3.263 | BI317002 | Signal Transduction | Protein Kinase | | |
| 88 | Gma.4470.1.S1_s_at | 3.26 | AW156251 | Unclassified - Protein | | | |
| | | | | with Unknown Function | | | |
| 89 | GmaAffx.11276.1.S1_s_at | 3.252 | AW567677 | Unclassified - | | | |
| | | | | Hypothetical Protein | | | |
| | | | | Supported by cDNA | | | |
| 90 | GmaAffx.54265.1.S1_s_at | 3.24 | BU546096 | Signal Transduction | Protein Kinase | | |
| 91 | GmaAffx.37908.1.A1_at | 3.229 | BU546116 | Signal Transduction | Protein Kinase | | |
| 92 | Gma.12867.1.A1_at | 3.18 | CD393518 | Unclassified - | | | |
| | | | | Hypothetical Protein | | | |
| | | | | Supported by cDNA | | | |
| 93 | GmaAffx.10340.1.S1_at | 3.178 | CA938421 | No Homology to Known | | | |
| | | | | Proteins | | | |
| 94 | GmaAffx.78434.1.S1_at | 3.177 | BG041904 | Unclassified - | | | Predicted membrane protein, |
| | | | | Hypothetical Protein | | | contains DoH and |
| | | | | NOT Supported by | | | Cytochrome b-561/ferric |
| | | | | cDNA | | | reductase transmembrane |
| | | | | | | | domains |
| 95 | Gma.13433.1.A1_at | 3.173 | CD398049 | No Homology to Known | | | |
| | | | | Proteins | | | |
| 96 | Gma.5629.2.S1_a_at | 3.165 | BG042866 | Disease & Defense | | | |
| 97 | Gma.2972.1.S1_at | 3.158 | AW350602 | Unclassified - | | | Phosphatidylinositol transfer |
| | | | | Hypothetical Protein | | | protein SEC14 and related |
| | | | | Supported by cDNA | | | proteins |
| 98 | GmaAffx.56563.1.S1_at | 3.133 | CA820002 | No Homology to Known | | | |
| | | | | Proteins | | | |
| 99 | Gma.368.1.S1_at | 3.124 | AW349594 | No Homology to Known | | | |
| | | | | Proteins | | | |
| 100 | GmaAffx.67477.2.S1_at | 3.116 | AW509184 | Cell Structure | | | |
| 101 | Gma.10307.1.S1_at | 3.114 | CA936845 | Unclassified - | | | |
| | | | | Hypothetical Protein | | | |
| | | | | NOT Supported by | | | |
| | | | | cDNA | | | |
| 102 | Gma.16308.1.S1_s_at | 3.061 | U64916.1 | Unclassified - | | | Copper chaperone |
| | | | | Hypothetical Protein | | | |
| | | | | NOT Supported by | | | |
| | | | | cDNA | | | |
| 103 | GmaAffx.45530.1.S1_at | 3.048 | BG789917 | Signal Transduction | Protein Kinase | | |
| 104 | GmaAffx.11858.1.A1_s_at | 3.047 | AW351023 | Metabolism | Phospholipid | | Glycosylphosphatidylinositol |
| | | | | | Biosynthesis | | specific phospholipase C |
| 105 | GmaAffx.90042.1.S1_at | 3.017 | CF805767 | Signal Transduction | Calcium Binding | | Calmodulin and related |
| | | | | | | | proteins (EF-Hand |
| | | | | | | | superfamily) |
| 106 | Gma.3777.1.S1_a_at | 3.008 | CD399954 | Unclassified - | | | |
| | | | | Hypothetical Protein | | | |
| | | | | Supported by cDNA | | | |
| 107 | GmaAffx.65419.1.S1_at | 3.003 | CD405955 | Signal Transduction | Protein Kinase | | |
| 108 | GmaAffx.40860.1.S1_at | 3.002 | BQ628787 | Disease & Defense | | | |
| 109 | Gma.8797.1.A1_at | 2.953 | BI969881 | Unclassified - | | | |
| | | | | Hypothetical Protein | | | |
| | | | | NOT Supported by | | | |
| | | | | cDNA | | | |
| 110 | GmaAffx.84266.1.S1_at | 2.952 | AI495798 | Signal Transduction | Protein Kinase | AtCIPKs | Serine/threonine protein |
| | | | | | | | kinase |
| 111 | GmaAffx.66979.1.S1_at | 2.944 | BI497616 | Signal Transduction | Protein Kinase | AtCIPKs | Serine/threonine protein |
| | | | | | | | kinase |
| 112 | GmaAffx.78720.1.S1_at | 2.93 | BM526650 | No Homology to Known | | | |
| | | | | Proteins | | | |
| 113 | GmaAffx.21211.1.S1_at | 2.919 | BU550606 | Protein Destination & | | | Molecular chaperone |
| | | | | Storage | | | (HSP90 family) |
| 114 | Gma.15495.1.A1_at | 2.918 | CD409942 | Unclassified - | | | Serine/threonine protein |
| | | | | Hypothetical Protein | | | kinase |
| | | | | NOT Supported by | | | |
| | | | | cDNA | | | |
| 115 | Gma.10334.1.S1_at | 2.918 | AW234479 | Unclassified - | | | |
| | | | | Hypothetical Protein | | | |
| | | | | NOT Supported by | | | |
| | | | | cDNA | | | |
| 116 | Gma.6302.1.S1_at | 2.916 | BI786195 | Cell Structure | | | Beta-glucosidase, lactase |
| | | | | | | | phlorizinhydrolase, and |
| | | | | | | | related proteins |
| 117 | Gma.12821.1.S1_s_at | 2.903 | CD391504 | No Homology to Known | | | |
| | | | | Proteins | | | |
| 118 | GmaAffx.87812.2.S1_at | 2.898 | BI971676 | Signal Transduction | Protein Kinase | | |
| 119 | GmaAffx.14179.1.S1_at | 2.888 | BM085107 | Unclassified - | | | SETA binding protein SB1 |
| | | | | Hypothetical Protein | | | and related proteins, contain |
| | | | | NOT Supported by | | | BTB/POZ domain |
| | | | | cDNA | | | |
| 120 | Gma.6288.1.S1_at | 2.888 | AW348556 | Transporter | | | Aquaporin (major intrinsic |
| | | | | | | | protein family) |
| 121 | GmaAffx.68088.1.S1_at | 2.883 | BG507880 | Signal Transduction | Protein Kinase | | |
| 122 | Gma.3988.1.S1_at | 2.879 | BE658819 | Unclassified - | | | |
| | | | | Hypothetical Protein | | | |
| | | | | Supported by cDNA | | | |
| 123 | GmaAffx.17811.1.S1_at | 2.879 | BI426517 | Transporter | | | Predicted transporter (major |
| | | | | | | | facilitator superfamily) |
| 124 | Gma.13027.1.A1_at | 2.877 | CD397394 | Transporter | | | Amino acid transporters |
| 125 | Gma.320.1.S1_at | 2.877 | AF019116.1 | Disease & Defense | | | |
| 126 | GmaAffx.10491.1.S1_at | 2.847 | AW598141 | No Homology to Known | | | |
| | | | | Proteins | | | |
| 127 | Gma.3777.1.S1_x_at | 2.847 | CD399954 | Unclassified - | | | |
| | | | | Hypothetical Protein | | | |
| | | | | Supported by cDNA | | | |
| 128 | GmaAffx.50677.1.S1_s_at | 2.839 | BI971744 | No Homology to Known | | | |
| | | | | Proteins | | | |
| 129 | Gma.9913.2.S1_a_at | 2.822 | CD390580 | Transcription | Transcription | WRKY | |
| | | | | | Factor | | |
| 130 | Gma.7970.1.S1_at | 2.816 | BI699189 | No Homology to Known | | | |
| | | | | Proteins | | | |
| 131 | GmaAffx.74573.1.S1_at | 2.809 | BU763133 | No Homology to Known | | | |
| | | | | Proteins | | | |
| 132 | GmaAffx.42583.1.A1_at | 2.8 | BG154898 | Transporter | Inorganic Solute | Fe(II) transporter | Fe2+/Zn2+ regulated |
| | | | | | Cotransporters | isolog family | transporter |
| 133 | Gma.8459.1.S1_at | 2.791 | AF249317.1 | Signal Transduction | Protein Kinase | | Serine/threonine protein |
| | | | | | | | kinase |
| 134 | GmaAffx.9944.1.S1_at | 2.781 | BM567882 | Cell Structure | | | |
| 135 | Gma.4832.1.A1_at | 2.765 | AW310458 | Signal Transduction | Protein Kinase | | |
| 136 | Gma.17559.1.S1_at | 2.764 | BG508388 | No Homology to Known | | | |
| | | | | Proteins | | | |
| 137 | GmaAffx.93534.1.S1_s_at | 2.76 | CF809259 | Unclassified - Protein | | | |
| | | | | with Unknown Function | | | |
| 138 | Gma.17576.1.S1_s_at | 2.747 | BG650176 | No Homology to Known | | | |
| | | | | Proteins | | | |
| 139 | GmaAffx.66589.1.S1_at | 2.74 | BE821501 | Transcription | Transcription | Polycomb Group | |
| | | | | | Factor | | |
| 140 | GmaAffx.60595.1.S1_at | 2.738 | BQ628072 | No Homology to Known | | | |
| | | | | Proteins | | | |
| 141 | Gma.5621.1.S1_at | 2.71 | BU579234 | Unclassified - | | | |
| | | | | Hypothetical Protein | | | |
| | | | | NOT Supported by | | | |
| | | | | cDNA | | | |
| 142 | GmaAffx.52050.1.S1_at | 2.707 | BG237350 | Transcription | Transcription | MYB | Transcription factor, Myb |
| | | | | | Factor | | superfamily |
| 143 | GmaAffx.85409.1.S1_at | 2.681 | BF598665 | Unclassified - | | | Copper chaperone |
| | | | | Hypothetical Protein | | | |
| | | | | NOT Supported by | | | |
| | | | | cDNA | | | |
| 144 | GmaAffx.88037.2.S1_at | 2.679 | CA783510 | Unclassified - | | | |
| | | | | Hypothetical Protein | | | |
| | | | | Supported by cDNA | | | |
| 145 | Gma.17605.3.S1_at | 2.678 | BQ079859 | Secondary Metabolism | Flavonoid | | |
| | | | | | Biosynthesis | | |
| 146 | GmaAffx.91346.1.S1_x_at | 2.634 | CF807071 | Unclassified - | | | |
| | | | | Hypothetical Protein | | | |
| | | | | Supported by cDNA | | | |
| 147 | GmaAffx.89999.1.S1_at | 2.613 | CF805724 | Disease & Defense | | | |
| 148 | Gma.17816.1.A1_at | 2.607 | BI970976 | Disease & Defense | | | |
| 149 | Gma.68.1.S1_at | 2.6 | AF357211.1 | Transcription | Transcription | AP2/EREBP | |
| | | | | | Factor | | |
| 150 | Gma.7921.1.S1_at | 2.59 | BM177567 | Unclassified - | | | Uncharacterized conserved |
| | | | | Hypothetical Protein | | | protein |
| | | | | Supported by cDNA | | | |
| 151 | GmaAffx.86745.1.S1_s_at | 2.587 | BI424960 | Disease & Defense | | | |
| 152 | GmaAffx.56917.1.S1_at | 2.564 | AI441130 | Signal Transduction | Protein Kinase | | Serine/threonine protein |
| | | | | | | | kinase |
| 153 | Gma.9013.1.S1_at | 2.558 | AF243368.1 | Disease & Defense | | | Glutathione S-transferase |
| 154 | GmaAffx.28927.1.S1_at | 2.545 | BU549173 | Transcription | Transcription | MYB | Transcription factor, Myb |
| | | | | | Factor | | superfamily |
| 155 | GmaAffx.87812.1.S1_at | 2.543 | AW831390 | Signal Transduction | Protein Kinase | | |
| 156 | GmaAffx.10590.1.S1_s_at | 2.537 | BU551319 | Signal Transduction | Protein Kinase | | |
| 157 | Gma.4169.1.S1_at | 2.53 | BQ473559 | Transporter | | | Copper chaperone |
| 158 | GmaAffx.91346.1.S1_at | 2.513 | CF807071 | Unclassified - | | | |
| | | | | Hypothetical Protein | | | |
| | | | | Supported by cDNA | | | |
| 159 | GmaAffx.4905.1.S1_at | 2.496 | BQ453808 | Metabolism | | | Predicted lipase |
| 160 | Gma.11257.1.S1_at | 2.477 | AW310476 | Cell Structure | | | |
| 161 | GmaAffx.16502.1.A1_at | 2.47 | BE659118 | No Homology to Known | | | |
| | | | | Proteins | | | |
| 162 | GmaAffx.90946.1.S1_at | 2.462 | CF808349 | Unclassified - | | | |
| | | | | Hypothetical Protein | | | |
| | | | | NOT Supported by | | | |
| | | | | cDNA | | | |
| 163 | GmaAffx.47716.1.A1_at | 2.449 | CD397037 | Unclassified - | | | |
| | | | | Hypothetical Protein | | | |
| | | | | NOT Supported by | | | |
| | | | | cDNA | | | |
| 164 | GmaAffx.77809.1.A1_at | 2.447 | BI969302 | Disease & Defense | | | |
| 165 | GmaAffx.7215.1.A1_at | 2.443 | AW351149 | Unclassified - | | | |
| | | | | Hypothetical Protein | | | |
| | | | | NOT Supported by | | | |
| | | | | cDNA | | | |
| 166 | GmaAffx.13625.1.A1_at | 2.436 | BU547925 | No Homology to Known | | | |
| | | | | Proteins | | | |
| 167 | Gma.14471.4.S1_x_at | 2.434 | BQ081409 | Transporter | | | Aquaporin (major intrinsic |
| | | | | | | | protein family) |
| 168 | Gma.3136.1.S1_at | 2.429 | BG839241 | Metabolism | Cytochrome P450 | CYP82C | Cytochrome P450 CYP2 |
| | | | | | | | subfamily |
| 169 | GmaAffx.11658.1.S1_at | 2.427 | AW184817 | Signal Transduction | Unclassified | | |
| 170 | Gma.6529.2.S1_s_at | 2.426 | BM309390 | Unclassified - | | | |
| | | | | Hypothetical Protein | | | |
| | | | | NOT Supported by | | | |
| | | | | cDNA | | | |
| 171 | GmaAffx.45241.1.S1_at | 2.422 | BG157181 | Unclassified - | | | Glutaredoxin and related |
| | | | | Hypothetical Protein | | | proteins |
| | | | | Supported by cDNA | | | |
| 172 | GmaAffx.38733.1.A1_at | 2.419 | BE657590 | No Homology to Known | | | |
| | | | | Proteins | | | |
| 173 | Gma.4396.1.S1_at | 2.417 | BQ081294 | No Homology to Known | | | |
| | | | | Proteins | | | |
| 174 | Gma.9013.1.S1_x_at | 2.407 | AF243368.1 | Disease & Defense | | | Glutathione S-transferase |
| 175 | GmaAffx.79390.3.S1_at | 2.406 | BU082743 | Transcription | Transcription | Basic Leucine | FOG: Armadillo/beta- |
| | | | | | Factor | Zipper (bZIP) | catenin-like repeats |
| 176 | Gma.742.1.A1_at | 2.395 | CD393482 | Unclassified - Protein | | | |
| | | | | with Unknown Function | | | |
| 177 | Gma.1521.1.S1_at | 2.394 | CD394504 | Cell Structure | | | Beta tubulin |
| 178 | GmaAffx.89629.1.S1_s_at | 2.387 | CK606095 | Unclassified - Protein | | | |
| | | | | with Unknown Function | | | |
| 179 | Gma.12465.1.S1_a_at | 2.384 | CD415130 | Unclassified - | | | |
| | | | | Hypothetical Protein | | | |
| | | | | Supported by cDNA | | | |
| 180 | GmaAffx.5264.1.S1_s_at | 2.381 | BM521512 | Disease & Defense | | | |
| 181 | GmaAffx.18236.1.S1_at | 2.378 | BG790288 | No Homology to Known | | | |
| | | | | Proteins | | | |
| 182 | Gma.11116.4.S1_s_at | 2.373 | CD408430 | Transporter | | | Aquaporin (major intrinsic |
| | | | | | | | protein family) |
| 183 | Gma.2352.1.S1_at | 2.369 | AI443511 | Unclassified - Protein | | | FOG: Predicted E3 ubiquitin |
| | | | | with Unknown Function | | | ligase |
| 184 | GmaAffx.81783.1.S1_at | 2.359 | AW101961 | Signal Transduction | Protein Kinase | | Serine/threonine protein |
| | | | | | | | kinase |
| 185 | GmaAffx.24352.1.S1_at | 2.358 | BU544287 | No Homology to Known | | | |
| | | | | Proteins | | | |
| 186 | Gma.6650.2.S1_at | 2.354 | BU762479 | Unclassified - Protein | | | |
| | | | | with Unknown Function | | | |
| 187 | GmaAffx.33259.1.S1_at | 2.351 | BQ785322 | Unclassified - | | | |
| | | | | Hypothetical Protein | | | |
| | | | | Supported by cDNA | | | |
| 188 | Gma.8301.2.S1_at | 2.349 | BU544524 | Signal Transduction | Calcium Binding | | |
| 189 | GmaAffx.56804.1.A1_at | 2.318 | CD415337 | Unclassified - | | | Uncharacterized conserved |
| | | | | Hypothetical Protein | | | protein, contains TraB |
| | | | | NOT Supported by | | | domain |
| | | | | cDNA | | | |
| 190 | Gma.6650.1.S1_at | 2.315 | BQ629283 | Unclassified - Protein | | | |
| | | | | with Unknown Function | | | |
| 191 | GmaAffx.86059.1.S1_at | 2.302 | BF598759 | No Homology to Known | | | |
| | | | | Proteins | | | |
| 192 | GmaAffx.74289.1.S1_at | 2.295 | CA852521 | Transcription | Transcription | MYB | Transcription factor, Myb |
| | | | | | Factor | | superfamily |
| 193 | Gma.3149.1.S1_a_at | 2.287 | BU544575 | No Homology to Known | | | |
| | | | | Proteins | | | |
| 194 | Gma.9925.1.S1_at | 2.284 | AW307455 | No Homology to Known | | | |
| | | | | Proteins | | | |
| 195 | GmaAffx.87526.1.S1_at | 2.283 | BM954581 | Unclassified - | | | |
| | | | | Hypothetical Protein | | | |
| | | | | Supported by cDNA | | | |
| 196 | GmaAffx.85264.1.S1_at | 2.276 | BE058913 | Metabolism | | | |
| 197 | Gma.3189.2.S1_at | 2.261 | BG511492 | Signal Transduction | MAPKK | | Mitogen-activated protein |
| | | | | | | | kinase kinase (MAPKK) |
| 198 | Gma.14966.1.A1_at | 2.259 | CD416359 | Intracellular Traffic | | | Aquaporin (major intrinsic |
| | | | | | | | protein family) |
| 199 | Gma.8077.1.S1_at | 2.259 | BU547877 | Cell Structure | | | |
| 200 | Gma.43.1.A1_at | 2.256 | L12258.1 | Transporter | | | Aquaporin (major intrinsic |
| | | | | | | | protein family) |
| 201 | GmaAffx.30344.1.S1_at | 2.255 | AW349341 | Metabolism | | | Lipid phosphate phosphatase |
| | | | | | | | and related enzymes of the |
| | | | | | | | PAP2 family |
| 202 | Gma.666.1.S1_at | 2.247 | AW310734 | Unclassified - Protein | Hormone Induced | | Voltage-gated shaker-like |
| | | | | with Unknown Function | | | K+ channel, subunit |
| | | | | | | | beta/KCNAB |
| 203 | GmaAffx.70634.1.S1_at | 2.247 | BQ079384 | Protein Destination & | | | FOG: Predicted E3 ubiquitin |
| | | | | Storage | | | ligase |
| 204 | Gma.17347.1.S1_s_at | 2.238 | AW597676 | Transcription | Transcription | NAC Domain | |
| | | | | | Factor | | |
| 205 | Gma.17805.1.A1_s_at | 2.238 | BI968729 | Disease & Defense | | | |
| 206 | Gma.9606.1.S1_at | 2.234 | AB086392.1 | Transporter | | | Sucrose transporter and |
| | | | | | | | related proteins |
| 207 | Gma.18088.1.S1_at | 2.232 | BI970254 | Unclassified - Protein | | | |
| | | | | with Unknown Function | | | |
| 208 | GmaAffx.91339.1.S1_at | 2.225 | CF807064 | Cell Structure | | | |
| 209 | Gma.5139.1.S1_at | 2.218 | AF243376.1 | Secondary Metabolism | | | Glutathione S-transferase |
| 210 | Gma.14846.1.A1_at | 2.218 | AW310268 | No Homology to Known | | | |
| | | | | Proteins | | | |
| 211 | Gma.5330.2.S1_at | 2.217 | BM092298 | Unclassified - | | | |
| | | | | Hypothetical Protein | | | |
| | | | | NOT Supported by | | | |
| | | | | cDNA | | | |
| 212 | GmaAffx.3697.1.S1_at | 2.212 | BE820647 | Disease & Defense | | | |
| 213 | GmaAffx.83638.1.S1_at | 2.203 | BU549896 | Unclassified - | | | |
| | | | | Hypothetical Protein | | | |
| | | | | NOT Supported by | | | |
| | | | | cDNA | | | |
| 214 | GmaAffx.90252.1.S1_s_at | 2.201 | CF805977 | Unclassified - | | | |
| | | | | Hypothetical Protein | | | |
| | | | | Supported by cDNA | | | |
| 215 | GmaAffx.56272.1.S1_at | 2.2 | BU761188 | No Homology to Known | | | |
| | | | | Proteins | | | |
| 216 | Gma.3810.1.S1_s_at | 2.19 | BI972063 | Disease & Defense | | | |
| 217 | Gma.4368.1.S1_at | 2.185 | BQ080689 | Unclassified - Protein | | | |
| | | | | with Unknown Function | | | |
| 218 | GmaAffx.58555.1.S1_at | 2.184 | BQ298550 | Metabolism | Cytochrome P450 | CYP82C | Cytochrome P450 CYP2 |
| | | | | | | | subfamily |
| 219 | GmaAffx.73185.1.S1_at | 2.183 | BI701903 | No Homology to Known | | | |
| | | | | Proteins | | | |
| 220 | Gma.11076.1.S1_at | 2.178 | BE658385 | Unclassified - | | | |
| | | | | Hypothetical Protein | | | |
| | | | | Supported by cDNA | | | |
| 221 | GmaAffx.62710.1.S1_at | 2.167 | BE473384 | Unclassified - | | | |
| | | | | Hypothetical Protein | | | |
| | | | | NOT Supported by | | | |
| | | | | cDNA | | | |
| 222 | Gma.5950.1.S1_s_at | 2.166 | BQ610563 | Disease & Defense | | | |
| 223 | GmaAffx.82105.1.S1_at | 2.164 | AI938690 | Transporter | | | Sucrose transporter and |
| | | | | | | | related proteins |
| 224 | Gma.15523.1.S1_s_at | 2.161 | BE475550 | Cell Structure | | | |
| 225 | GmaAffx.50656.1.A1_at | 2.159 | BI970755 | No Homology to Known | | | |
| | | | | Proteins | | | |
| 226 | GmaAffx.31548.1.S1_at | 2.158 | BG044632 | Transcription | Transcription | Unclassified | Transcription factor, Myb |
| | | | | | Factor | | superfamily |
| 227 | GmaAffx.43291.1.S1_at | 2.148 | AI442332 | Metabolism | | | Lipid phosphate phosphatase |
| | | | | | | | and related enzymes of the |
| | | | | | | | PAP2 family |
| 228 | Gma.14802.1.S1_at | 2.144 | AW201116 | Unclassified - Protein | | | FOG: Ankyrin repeat |
| | | | | with Unknown Function | | | |
| 229 | GmaAffx.77651.1.S1_at | 2.138 | BF010272 | Unclassified - Protein | Hormone Induced | | Zinc-binding oxidoreductase |
| | | | | with Unknown Function | | | |
| 230 | GmaAffx.87547.2.S1_at | 2.133 | BQ080473 | Cell Structure | | | Beta-glucosidase, lactase |
| | | | | | | | phlorizinhydrolase, and |
| | | | | | | | related proteins |
| 231 | Gma.17305.1.S1_at | 2.132 | AW351319 | Unclassified - Protein | | | |
| | | | | with Unknown Function | | | |
| 232 | GmaAffx.93073.1.S1_s_at | 2.121 | CF808798 | Cell Structure | | | |
| 233 | GmaAffx.31180.1.A1_at | 2.118 | BE820254 | No Homology to Known | | | |
| | | | | Proteins | | | |
| 234 | GmaAffx.30428.1.S1_at | 2.113 | BE824410 | Protein Destination & | | | Molecular chaperones |
| | | | | Storage | | | HSP70/HSC70, HSP70 |
| | | | | | | | superfamily |
| 235 | GmaAffx.671.1.S1_at | 2.112 | CA785167 | Cell Structure | | | |
| 236 | GmaAffx.91185.1.S1_at | 2.103 | CF806910 | Cell Structure | | | Pectin acetylesterase and |
| | | | | | | | similar proteins |
| 237 | GmaAffx.79888.1.S1_at | 2.097 | AI988123 | Signal Transduction | Protein Kinase | | |
| 238 | GmaAffx.43620.1.S1_at | 2.089 | AW184956 | No Homology to Known | | | |
| | | | | Proteins | | | |
| 239 | Gma.5610.1.S1_at | 2.089 | AW348190 | Unclassified - | | | |
| | | | | Hypothetical Protein | | | |
| | | | | Supported by cDNA | | | |
| 240 | Gma.17586.1.S1_at | 2.079 | BU546955 | Unclassified - | | | Reticulon |
| | | | | Hypothetical Protein | | | |
| | | | | Supported by cDNA | | | |
| 241 | GmaAffx.3363.1.S1_at | 2.074 | BE347006 | Unclassified - | | | |
| | | | | Hypothetical Protein | | | |
| | | | | NOT Supported by | | | |
| | | | | cDNA | | | |
| 242 | Gma.12465.2.S1_at | 2.073 | BE611098 | Unclassified - | | | |
| | | | | Hypothetical Protein | | | |
| | | | | Supported by cDNA | | | |
| 243 | GmaAffx.57341.1.S1_at | 2.067 | AW277978 | Transporter | | | Amino acid transporters |
| 244 | GmaAffx.77404.1.S1_at | 2.067 | CD393010 | Intracellular Traffic | | | GTPase Rab11/YPT3, small |
| | | | | | | | G protein superfamily |
| 245 | Gma.869.1.S1_at | 2.061 | BE822531 | Protein Destination & | | | Serine carboxypeptidases |
| | | | | Storage | | | (lysosomal cathepsin A) |
| 246 | Gma.3576.2.S1_a_at | 2.061 | BI316058 | Unclassified - Protein | | | Diadenosine polyphosphate |
| | | | | with Unknown Function | | | hydrolase and related |
| | | | | | | | proteins of the histidine triad |
| | | | | | | | (HIT) family |
| 247 | Gma.612.1.A1_at | 2.06 | AI442942 | Transcription | Transcription | AP2/EREBP | |
| | | | | | Factor | | |
| 248 | Gma.3555.1.S1_at | 2.059 | BU764259 | Unclassified - | | | |
| | | | | Hypothetical Protein | | | |
| | | | | NOT Supported by | | | |
| | | | | cDNA | | | |
| 249 | GmaAffx.8013.1.S1_at | 2.053 | BE609266 | Unclassified - | | | |
| | | | | Hypothetical Protein | | | |
| | | | | NOT Supported by | | | |
| | | | | cDNA | | | |
| 250 | Gma.10441.1.S1_s_at | 2.031 | BE803701 | Unclassified - Protein | | | |
| | | | | with Unknown Function | | | |
| 251 | GmaAffx.47891.1.S1_s_at | 2.03 | BQ785880 | Cell Structure | | | |
| 252 | GmaAffx.79401.1.S1_at | 2.027 | CA936959 | Transcription | Transcription | bZIP | |
| | | | | | Factor | | |
| 253 | GmaAffx.70056.1.S1_at | 2.018 | BU765705 | No Homology to Known | | | |
| | | | | Proteins | | | |
| 254 | GmaAffx.18518.1.S1_at | 2.016 | BI700329 | Cell Structure | | | |
| 255 | GmaAffx.46974.1.S1_at | 2.014 | BU081847 | Signal Transduction | Protein Kinase | | |
| 256 | Gma.13613.1.A1_at | 2.012 | CD401987 | No Homology to Known | | | |
| | | | | Proteins | | | |
| 257 | GmaAffx.23953.1.S1_s_at | 2.005 | BE658262 | Transporter | | | |
| 258 | GmaAffx.45853.1.S1_at | 2.005 | BQ081229 | Unclassified - Protein | | | |
| | | | | with Unknown Function | | | |
| 259 | Gma.13973.1.A1_at | 2.004 | CD412168 | No Homology to Known | | | |
| | | | | Proteins | | | |
| 260 | GmaAffx.72630.1.S1_at | 2.003 | BG239729 | Unclassified - | | | FOG: TPR repeat |
| | | | | Hypothetical Protein | | | |
| | | | | NOT Supported by | | | |
| | | | | cDNA | | | |
| 260 | GmaAffx.72630.1.S1_at | 2.003 | BG239729 | Unclassified - | | | Thioredoxin |
| | | | | Hypothetical Protein | | | |
| | | | | NOT Supported by | | | |
| | | | | cDNA | | | |
| 261 | Gma.6803.2.S1_s_at | 0.5 | BU082740 | Disease & Defense | | | |
| 262 | GmaAffx.45003.1.S1_at | 0.499 | BI968048 | Unclassified - | | | Uncharacterized conserved |
| | | | | Hypothetical Protein | | | protein |
| | | | | Supported by cDNA | | | |
| 263 | Gma.2952.1.S1_at | 0.499 | BM270250 | No Homology to Known | | | |
| | | | | Proteins | | | |
| 264 | GmaAffx.28397.1.S1_at | 0.499 | BM568229 | Metabolism | | | |
| 265 | GmaAffx.71944.1.S1_at | 0.498 | AW351155 | Unclassified - | | | |
| | | | | Hypothetical Protein | | | |
| | | | | Supported by cDNA | | | |
| 266 | Gma.6375.1.S1_at | 0.498 | BQ610190 | Unclassified - | | | AAA+-type ATPase |
| | | | | Hypothetical Protein | | | |
| | | | | NOT Supported by | | | |
| | | | | cDNA | | | |
| 267 | Gma.11028.2.S1_s_at | 0.498 | CA782711 | Metabolism | | | Glyoxylate/hydroxypyruvate |
| | | | | | | | reductase (D-isomer-specific |
| | | | | | | | 2-hydroxy acid |
| | | | | | | | dehydrogenase superfamily) |
| 268 | Gma.1528.1.S1_at | 0.498 | CD403623 | Transcription | Transcription | Basic Helix-Loop- | |
| | | | | | Factor | Helix (bHLH) | |
| 269 | GmaAffx.85469.1.S1_at | 0.497 | BI786397 | Unclassified - | | | |
| | | | | Hypothetical Protein | | | |
| | | | | NOT Supported by | | | |
| | | | | cDNA | | | |
| 270 | GmaAffx.90069.1.S1_at | 0.494 | CF805794 | Unclassified - | | | Leucine rich repeat proteins, |
| | | | | Hypothetical Protein | | | some proteins contain F-box |
| | | | | Supported by cDNA | | | |
| 271 | GmaAffx.79516.1.A1_at | 0.494 | CD400596 | Transcription | Transcription | MYB | |
| | | | | | Factor | | |
| 272 | GmaAffx.37930.1.A1_at | 0.494 | BU547709 | Cell Structure | | | |
| 273 | Gma.3744.1.S1_at | 0.494 | CD417438 | Unclassified - Protein | | | |
| | | | | With Unknown | | | |
| | | | | Function | | | |
| 274 | Gma.1642.3.S1_s_at | 0.493 | BM891895 | Unclassified - | | | |
| | | | | Hypothetical Protein | | | |
| | | | | Supported by cDNA | | | |
| 275 | Gma.7371.2.S1_a_at | 0.493 | BQ741076 | Cell Structure | | | Dynein light chain type 1 |
| 276 | Gma.16521.1.S1_at | 0.493 | BE609300 | Unclassified - | | | Uncharacterized conserved |
| | | | | Hypothetical Protein | | | protein |
| | | | | Supported by cDNA | | | |
| 277 | Gma.13335.1.S1_at | 0.492 | CD417489 | Unclassified - | | | |
| | | | | Hypothetical Protein | | | |
| | | | | Supported by cDNA | | | |
| 278 | GmaAffx.7302.2.A1_s_at | 0.488 | BE820704 | No Homology to Known | | | |
| | | | | Proteins | | | |
| 279 | Gma.13039.1.A1_at | 0.488 | CD399750 | Unclassified - | | | |
| | | | | Hypothetical Protein | | | |
| | | | | NOT Supported by | | | |
| | | | | cDNA | | | |
| 280 | GmaAffx.35729.1.S1_s_at | 0.488 | BE806047 | Unclassified - | | | Predicted E3 ubiquitin ligase |
| | | | | Hypothetical Protein | | | |
| | | | | Supported by cDNA | | | |
| 281 | GmaAffx.81502.2.S1_at | 0.486 | BG041291 | Unclassified - | | | Uncharacterized conserved |
| | | | | Hypothetical Protein | | | protein |
| | | | | NOT Supported by | | | |
| | | | | cDNA | | | |
| 282 | GmaAffx.63115.1.A1_at | 0.485 | BE823736 | Unclassified - | | | |
| | | | | Hypothetical Protein | | | |
| | | | | NOT Supported by | | | |
| | | | | cDNA | | | |
| 283 | Gma.4315.1.S1_at | 0.485 | BM886786 | Unclassified - Protein | | | |
| | | | | with Unknown Function | | | |
| 284 | Gma.3591.1.S1_a_at | 0.485 | BE440266 | Secondary Metabolism | Ethylene | | Iron/ascorbate family |
| | | | | | Biosynthesis | | oxidoreductases |
| 285 | GmaAffx.33527.1.S1_at | 0.485 | BU761195 | No Homology to Known | | | |
| | | | | Proteins | | | |
| 286 | GmaAffx.53069.2.S1_at | 0.483 | AW309012 | No Homology to Known | | | |
| | | | | Proteins | | | |
| 287 | GmaAffx.91731.1.S1_s_at | 0.483 | CF807456 | Secondary Metabolism | Ethylene | | Iron/ascorbate family |
| | | | | | Biosynthesis | | oxidoreductases |
| 288 | GmaAffx.90242.1.S1_s_at | 0.483 | CF805967 | No Homology to Known | | | |
| | | | | Proteins | | | |
| 289 | Gma.7422.1.S1_at | 0.482 | BQ612005 | Transporter | | | |
| 290 | Gma.3744.2.S1_a_at | 0.481 | BI892693 | Unclassified - Protein | | | |
| | | | | With Unknown | | | |
| | | | | Function | | | |
| 291 | Gma.13418.3.S1_at | 0.481 | CA938391 | Transporter | | | |
| 292 | GmaAffx.73857.1.S1_at | 0.48 | BM885538 | Unclassified - | | | |
| | | | | Hypothetical Protein | | | |
| | | | | Supported by cDNA | | | |
| 293 | GmaAffx.50635.2.S1_at | 0.48 | AW733509 | Metabolism | Cytochrome P450 | CYP707A | Cytochrome P450 |
| | | | | | | | CYP4/CYP19/CYP26 |
| | | | | | | | subfamilies |
| 294 | GmaAffx.83103.2.S1_at | 0.48 | BE806704 | Unclassified - | | | |
| | | | | Hypothetical Protein | | | |
| | | | | NOT Supported by | | | |
| | | | | cDNA | | | |
| 295 | GmaAffx.86667.1.S1_at | 0.48 | CA938472 | Unclassified - | | | |
| | | | | Hypothetical Protein | | | |
| | | | | Supported by cDNA | | | |
| 296 | GmaAffx.93157.1.S1_s_at | 0.479 | CF808882 | Metabolism | | | Aldo/keto reductase family |
| | | | | | | | proteins |
| 297 | GmaAffx.37803.2.S1_at | 0.478 | BE347017 | No Homology to Known | | | |
| | | | | Proteins | | | |
| 298 | GmaAffx.71032.1.S1_at | 0.477 | AW278168 | Unclassified - | | | |
| | | | | Hypothetical Protein | | | |
| | | | | NOT Supported by | | | |
| | | | | cDNA | | | |
| 299 | Gma.17007.1.S1_at | 0.477 | BE820533 | No Homology to Known | | | |
| | | | | Proteins | | | |
| 300 | GmaAffx.76295.1.S1_at | 0.476 | BE347326 | Unclassified - Protein | Hormone Induced | | |
| | | | | with Unknown Function | | | |
| 301 | GmaAffx.74390.1.A1_at | 0.476 | BU544169 | Protein Destination & | | | Molecular chaperone (DnaJ |
| | | | | Storage | | | superfamily) |
| 302 | Gma.16842.1.A1_at | 0.475 | CD392652 | No Homology to Known | | | |
| | | | | Proteins | | | |
| 303 | Gma.4874.1.A1_at | 0.475 | AI938293 | No Homology to Known | | | |
| | | | | Proteins | | | |
| 304 | GmaAffx.25612.1.S1_at | 0.475 | AW317577 | No Homology to Known | | | |
| | | | | Proteins | | | |
| 305 | GmaAffx.30593.1.S1_at | 0.474 | BE803365 | Unclassified - | | | |
| | | | | Hypothetical Protein | | | |
| | | | | Supported by cDNA | | | |
| 306 | GmaAffx.33677.1.S1_at | 0.474 | CA782582 | No Homology to Known | | | |
| | | | | Proteins | | | |
| 307 | GmaAffx.76699.1.S1_at | 0.473 | AW760679 | Transcription | Transcription | Basic Helix-Loop- | |
| | | | | | Factor | Helix (bHLH) | |
| 308 | Gma.4340.2.S1_a_at | 0.473 | BE611760 | Secondary Metabolism | | | Hydroxyindole-O- |
| | | | | | | | methyltransferase and related |
| | | | | | | | SAM-dependent |
| | | | | | | | methyltransferases |
| 309 | GmaAffx.38961.1.S1_at | 0.473 | BU081740 | Transcription | Transcription | MYB | Transcription factor, Myb |
| | | | | | Factor | | superfamily |
| 310 | GmaAffx.78358.2.S1_at | 0.472 | BU762686 | Metabolism | | | |
| 311 | Gma.2565.1.S1_at | 0.471 | BG652533 | Unclassified - | | | |
| | | | | Hypothetical Protein | | | |
| | | | | Supported by cDNA | | | |
| 312 | GmaAffx.53033.1.S1_at | 0.471 | BG046837 | Transcription | Transcription | AP2/EREBP | |
| | | | | | Factor | | |
| 313 | GmaAffx.79730.1.S1_at | 0.471 | BF325482 | Transcription | Transcription | Basic Helix-Loop- | |
| | | | | | Factor | Helix (bHLH) | |
| 314 | Gma.15625.1.S1_a_at | 0.467 | U77678.1 | Metabolism | Amino Acid | | Asparagine synthase |
| | | | | | Biosynthesis/Metabolism | | (glutamine-hydrolyzing) |
| 315 | GmaAffx.59808.1.S1_at | 0.467 | BM886228 | Transporter | | | Predicted transporter (major |
| | | | | | | | facilitator superfamily) |
| 316 | Gma.16196.1.S1_at | 0.467 | AW350888 | Transcription | Transcription | Unclassified | |
| | | | | | Factor | | |
| 317 | Gma.12641.1.A1_at | 0.467 | CD393495 | No Homology to Known | | | |
| | | | | Proteins | | | |
| 318 | GmaAffx.57469.1.S1_at | 0.467 | AW351054 | Unclassified - | | | |
| | | | | Hypothetical Protein | | | |
| | | | | Supported by cDNA | | | |
| 319 | GmaAffx.23475.2.S1_s_at | 0.467 | AW620563 | Unclassified - | | | |
| | | | | Hypothetical Protein | | | |
| | | | | Supported by cDNA | | | |
| 320 | Gma.7256.1.S1_at | 0.466 | L01447.1 | Transcription | Transcription | Basic Leucine | |
| | | | | | Factor | Zipper (bZIP) | |
| 321 | Gma.13103.1.S1_at | 0.466 | CD392133 | Unclassified - | | | |
| | | | | Hypothetical Protein | | | |
| | | | | NOT Supported by | | | |
| | | | | cDNA | | | |
| 322 | Gma.4643.1.S1_at | 0.466 | BU547906 | Transcription | Transcription | NAC Domain | |
| | | | | | Factor | | |
| 323 | Gma.11665.2.A1_at | 0.465 | BU545959 | No Homology to Known | | | |
| | | | | Proteins | | | |
| 324 | GmaAffx.74256.1.S1_at | 0.463 | BE057293 | No Homology to Known | | | |
| | | | | Proteins | | | |
| 325 | Gma.7040.1.A1_at | 0.462 | BE346424 | Transcription | Transcription | AP2/EREBP | |
| | | | | | Factor | | |
| 326 | Gma.13146.1.S1_at | 0.461 | BM731716 | Unclassified - | | | |
| | | | | Hypothetical Protein | | | |
| | | | | Supported by cDNA | | | |
| 327 | GmaAffx.48042.1.S1_at | 0.461 | AI960210 | Transporter | | | Aquaporin (major intrinsic |
| | | | | | | | protein family) |
| 328 | GmaAffx.27488.1.S1_at | 0.459 | AW311080 | Signal Transduction | MAPKKK | Raf | Tyrosine kinase specific for |
| | | | | | | | activated (GTP-bound) |
| | | | | | | | p21cdc42Hs |
| 329 | GmaAffx.71386.1.S1_at | 0.458 | AW704747 | Protein Destination & | | | Molecular chaperone (DnaJ |
| | | | | Storage | | | superfamily) |
| 330 | GmaAffx.6017.1.A1_at | 0.458 | BU550467 | Unclassified - | | | |
| | | | | Hypothetical Protein | | | |
| | | | | NOT Supported by | | | |
| | | | | cDNA | | | |
| 331 | GmaAffx.68318.1.S1_at | 0.457 | BU545357 | Transcription | Transcription | AUX/1AA | |
| | | | | | Factor | | |
| 332 | GmaAffx.87295.1.S1_at | 0.455 | BM519660 | No Homology to Known | | | |
| | | | | Proteins | | | |
| 333 | Gma.2718.1.S1_at | 0.455 | CD406302 | Transporter | | | Multidrug resistance- |
| | | | | | | | associated |
| | | | | | | | protein/mitoxantrone |
| | | | | | | | resistance protein, ABC |
| | | | | | | | superfamily |
| 334 | GmaAffx.53256.1.S1_at | 0.454 | BU545086 | Unclassified - | | | |
| | | | | Hypothetical Protein | | | |
| | | | | Supported by cDNA | | | |
| 335 | GmaAffx.53069.1.S1_at | 0.454 | AW733700 | Unclassified - | | | |
| | | | | Hypothetical Protein | | | |
| | | | | NOT Supported by | | | |
| | | | | cDNA | | | |
| 336 | GmaAffx.93308.1.S1_at | 0.454 | CF809033 | Protein Destination & | | | Glutathione S-transferase |
| | | | | Storage | | | |
| 337 | GmaAffx.53099.1.S1_at | 0.454 | BG043340 | Unclassified - | | | Alpha-L-fucosidase |
| | | | | Hypothetical Protein | | | |
| | | | | NOT Supported by | | | |
| | | | | cDNA | | | |
| 338 | GmaAffx.13730.1.S1_at | 0.453 | BI316755 | Transcription | Transcription | Basic Helix-Loop- | |
| | | | | | Factor | Helix (bHLH) | |
| 339 | GmaAffx.70435.2.S1_at | 0.453 | AW705853 | Transcription | Transcription | Basic Helix-Loop- | |
| | | | | | Factor | Helix (bHLH) | |
| 340 | Gma.6659.1.S1_at | 0.452 | BQ629137 | Unclassified - Protein | | | Protein involved in |
| | | | | with Unknown Function | | | membrane traffic |
| | | | | | | | (YOP1/TB2/DP1/HVA22 |
| | | | | | | | family) |
| 341 | GmaAffx.55657.1.S1_at | 0.451 | BM885600 | No Homology to Known | | | |
| | | | | Proteins | | | |
| 342 | GmaAffx.76696.1.A1_at | 0.451 | BE824386 | No Homology to Known | | | |
| | | | | Proteins | | | |
| 343 | GmaAffx.90899.1.S1_at | 0.447 | CF806624 | Unclassified - | | | |
| | | | | Hypothetical Protein | | | |
| | | | | NOT Supported by | | | |
| | | | | cDNA | | | |
| 344 | GmaAffx.5232.1.S1_at | 0.447 | BM309521 | Unclassified - Protein | | | |
| | | | | with Unknown Function | | | |
| 345 | Gma.17686.1.S1_at | 0.447 | BF597378 | Metabolism | Cytochrome P450 | CYP76C | Cytochrome P450 CYP2 |
| | | | | | | | subfamily |
| 346 | Gma.1195.1.S1_s_at | 0.447 | AI442801 | Metabolism | | | Predicted flavoprotein |
| 347 | GmaAffx.73303.1.S1_at | 0.446 | BE190156 | Unclassified - | | | Predicted membrane protein |
| | | | | Hypothetical Protein | | | |
| | | | | Supported by cDNA | | | |
| 348 | Gma.6804.1.S1_at | 0.446 | BE657581 | Protein Destination & | | | Aspartyl protease |
| | | | | Storage | | | |
| 349 | GmaAffx.74160.1.S1_at | 0.446 | BQ740845 | Protein Destination & | | | |
| | | | | Storage | | | |
| 350 | Gma.1951.2.S1_at | 0.445 | CA783485 | Protein Destination & | | | Serine carboxypeptidases |
| | | | | Storage | | | |
| 351 | Gma.10591.2.S1_at | 0.444 | CD414199 | Metabolism | | | Glycine cleavage system H |
| | | | | | | | protein (lipoate-binding) |
| 352 | Gma.5760.2.S1_at | 0.444 | BQ298381 | Disease & Defense | | | |
| 353 | GmaAffx.25912.1.S1_at | 0.443 | BU545028 | Unclassified - Protein | | | |
| | | | | with Unknown Function | | | |
| 354 | GmaAffx.74693.1.S1_at | 0.442 | CD406171 | Metabolism | | | |
| 355 | GmaAffx.69307.1.S1_at | 0.442 | BQ473855 | No Homology to Known | | | |
| | | | | Proteins | | | |
| 356 | Gma.2066.1.S1_a_at | 0.44 | BG363704 | No Homology to Known | | | |
| | | | | Proteins | | | |
| 357 | GmaAffx.26683.1.A1_at | 0.439 | BE820379 | No Homology to Known | | | |
| | | | | Proteins | | | |
| 358 | Gma.11194.2.S1_at | 0.438 | BM886635 | Energy | | | Thioredoxin |
| 359 | Gma.10100.1.S1_at | 0.438 | BE023275 | No Homology to Known | | | |
| | | | | Proteins | | | |
| 360 | Gma.3880.1.S1_at | 0.437 | BE659305 | Unclassified - | | | |
| | | | | Hypothetical Protein | | | |
| | | | | NOT Supported by | | | |
| | | | | cDNA | | | |
| 361 | Gma.13241.1.S1_at | 0.434 | CD395739 | Unclassified - | | | |
| | | | | Hypothetical Protein | | | |
| | | | | NOT Supported by | | | |
| | | | | cDNA | | | |
| 362 | GmaAffx.13016.1.S1_at | 0.434 | BF069324 | No Homology to Known | | | |
| | | | | Proteins | | | |
| 363 | Gma.2827.1.S1_at | 0.433 | BG237560 | Unclassified - | | | FOG: PPR repeat |
| | | | | Hypothetical Protein | | | |
| | | | | Supported by cDNA | | | |
| 364 | GmaAffx.56319.1.S1_at | 0.433 | BE802287 | Metabolism | | | Uncharacterized conserved |
| | | | | | | | protein |
| 365 | GmaAffx.26667.1.S1_at | 0.432 | BE806932 | No Homology to Known | | | |
| | | | | Proteins | | | |
| 366 | Gma.6129.2.S1_s_at | 0.432 | AW186006 | Protein Synthesis | | | Translation initiation factor |
| | | | | | | | 5A (eIF-5A) |
| 367 | GmaAffx.8304.1.A1_at | 0.431 | BE822695 | Unclassified - | | | Possible oxidoreductase |
| | | | | Hypothetical Protein | | | |
| | | | | Supported by cDNA | | | |
| 368 | Gma.4305.1.S1_at | 0.431 | AW350687 | Disease & Defense | | | |
| 369 | GmaAffx.87900.1.S1_at | 0.429 | BI892943 | Protein Destination & | | | FKBP-type peptidyl-prolyl |
| | | | | Storage | | | cis-trans isomerase |
| 370 | GmaAffx.90113.1.S1_at | 0.428 | CF806004 | Protein Destination & | Storage Protein | | Uncharacterized conserved |
| | | | | Storage | | | protein |
| 371 | GmaAffx.59210.1.S1_at | 0.428 | BI967182 | Secondary Metabolism | Ethylene | | Iron/ascorbate family |
| | | | | | Biosynthesis | | oxidoreductases |
| 372 | Gma.7227.1.S1_at | 0.428 | BU551112 | Protein Destination & | | | Ubiquitin-protein ligase |
| | | | | Storage | | | |
| 373 | Gma.10940.1.S1_at | 0.426 | BU550016 | Unclassified - | | | Metalloendopeptidase family - |
| | | | | Hypothetical Protein | | | saccharolysin & thimet |
| | | | | Supported by cDNA | | | oligopeptidase |
| 374 | Gma.2887.2.S1_x_at | 0.426 | BI701520 | Secondary Metabolism | | | Phenylalanine and histidine |
| | | | | | | | ammonia-lyase |
| 375 | Gma.17191.1.S1_at | 0.426 | BG237724 | Metabolism | | | UDP-glucuronosyl and UDP- |
| | | | | | | | glucosyl transferase |
| 376 | GmaAffx.84144.1.A1_s_at | 0.426 | CD414061 | No Homology to Known | | | |
| | | | | Proteins | | | |
| 377 | GmaAffx.58073.1.S1_at | 0.423 | CA852105 | Unclassified - | | | |
| | | | | Hypothetical Protein | | | |
| | | | | Supported by cDNA | | | |
| 378 | Gma.7549.1.S1_at | 0.423 | BG508637 | Unclassified - Protein | | | |
| | | | | with Unknown Function | | | |
| 379 | GmaAffx.69792.1.S1_s_at | 0.423 | BU550596 | Transcription | Transcription | AP2/EREBP | |
| | | | | | Factor | | |
| 380 | Gma.3165.1.S1_at | 0.422 | CD406755 | Transcription | Transcription | Homeodomain | Transcription factor HEX, |
| | | | | | Factor | | contains HOX and HALZ |
| | | | | | | | domains |
| 381 | Gma.10393.1.S1_at | 0.422 | BE804608 | Unclassified - | | | Endoplasmic reticulum |
| | | | | Hypothetical Protein | | | protein EP58, contains |
| | | | | NOT Supported by | | | filamin rod domain and |
| | | | | cDNA | | | KDEL motif |
| 382 | GmaAffx.948.1.S1_at | 0.421 | BG155389 | Protein Destination & | | | Ubiquitin fusion-degradation |
| | | | | Storage | | | protein |
| 383 | GmaAffx.21041.1.A1_at | 0.421 | AW350819 | No Homology to Known | | | |
| | | | | Proteins | | | |
| 384 | GmaAffx.1301.68.S1_s_at | 0.421 | BI974374 | Intracellular Traffic | | | Annexin |
| 385 | GmaAffx.15471.1.S1_at | 0.42 | BQ611747 | Transcription | Transcription | MYB | Transcription factor, Myb |
| | | | | | Factor | | superfamily |
| 386 | GmaAffx.6017.2.S1_at | 0.419 | BI469662 | Unclassified - | | | |
| | | | | Hypothetical Protein | | | |
| | | | | NOT Supported by | | | |
| | | | | cDNA | | | |
| 387 | Gma.10536.1.S1_at | 0.419 | BG508300 | No Homology to Known | | | |
| | | | | Proteins | | | |
| 388 | PsAffx.psHB026xH01f_s_at | 0.419 | | | | | |
| 389 | Gma.16947.1.S1_at | 0.419 | BG155375 | Unclassified - | | | |
| | | | | Hypothetical Protein | | | |
| | | | | Supported by cDNA | | | |
| 390 | Gma.1490.1.S1_at | 0.418 | AB000097.1 | Cell Structure | | | Chitinase |
| 391 | Gma.8501.1.S1_at | 0.418 | AF184278.1 | Transcription | Transcription | Homeodomain | Transcription factor HEX, |
| | | | | | Factor | | contains HOX and HALZ |
| | | | | | | | domains |
| 392 | GmaAffx.12014.1.A1_at | 0.418 | CA802431 | Unclassified - | | | NADH pyrophosphatase 1 of |
| | | | | Hypothetical Protein | | | the Nudix family of |
| | | | | Supported by cDNA | | | hydrolases |
| 393 | GmaAffx.83226.1.S1_at | 0.417 | CA852250 | Transcription | Transcription | Unclassified | |
| | | | | | Factor | | |
| 394 | GmaAffx.52391.1.S1_at | 0.416 | AW349563 | Unclassified - | | | |
| | | | | Hypothetical Protein | | | |
| | | | | NOT Supported by | | | |
| | | | | cDNA | | | |
| 395 | GmaAffx.34657.1.A1_s_at | 0.415 | AW351012 | No Homology to Known | | | |
| | | | | Proteins | | | |
| 396 | Gma.11731.1.A1_at | 0.414 | CD392320 | Unclassified - Protein | | | |
| | | | | with Unknown Function | | | |
| 397 | GmaAffx.74500.1.A1_at | 0.413 | BU551117 | No Homology to Known | | | |
| | | | | Proteins | | | |
| 398 | GmaAffx.1301.59.S1_at | 0.412 | BI974591 | Intracellular Traffic | | | Annexin |
| 399 | Gma.3578.1.S1_s_at | 0.412 | AW156522 | Disease & Defense | | | Acyl-CoA synthetase |
| 400 | Gma.5513.1.S1_at | 0.412 | AW310453 | Metabolism | | | Haloacid dehalogenase-like |
| | | | | | | | hydrolase |
| 401 | Gma.5294.1.S1_s_at | 0.411 | BE658252 | Unclassified - | | | |
| | | | | Hypothetical Protein | | | |
| | | | | Supported by cDNA | | | |
| 402 | GmaAffx.84566.3.S1_s_at | 0.411 | BG045916 | Transcription | Transcription | MYB | Transcription factor, Myb |
| | | | | | Factor | | superfamily |
| 403 | GmaAffx.11999.1.S1_at | 0.411 | AW567661 | No Homology to Known | | | |
| | | | | Proteins | | | |
| 404 | GmaAffx.5255.1.S1_at | 0.41 | BG362929 | Transcription | Transcription | AP2/EREBP | |
| | | | | | Factor | | |
| 405 | GmaAffx.25068.2.A1_at | 0.409 | CD413732 | Metabolism | Amino Acid | | |
| | | | | | Biosynthesis/Metabolism | | |
| 406 | Gma.595.1.S1_at | 0.407 | AB004271.1 | Metabolism | | | |
| 407 | Gma.5513.1.S1_x_at | 0.406 | AW310453 | Metabolism | | | Haloacid dehalogenase-like |
| | | | | | | | hydrolase |
| 408 | GmaAffx.86613.2.S1_at | 0.404 | BE024005 | Metabolism | | | Glutaredoxin and related |
| | | | | | | | proteins |
| 409 | Gma.4099.1.S1_at | 0.404 | BG651846 | Metabolism | | | Beta-galactosidase |
| 410 | GmaAffx.60479.1.S1_at | 0.403 | BG726023 | Transcription | Transcription | Homeodomain | Transcription factor MEIS1 |
| | | | | | Factor | | and related HOX domain |
| | | | | | | | proteins |
| 411 | Gma.13019.1.A1_s_at | 0.402 | CD392967 | Secondary Metabolism | | | Hydroxyindole-O- |
| | | | | | | | methyltransferase and related |
| | | | | | | | SAM-dependent |
| | | | | | | | methyltransferases |
| 412 | Gma.6895.1.S1_a_at | 0.402 | BU545788 | Metabolism | | | Alanine-glyoxylate |
| | | | | | | | aminotransferase AGT2 |
| 413 | GmaAffx.31838.1.S1_at | 0.402 | BU547835 | Secondary Metabolism | Flavonoid | | Flavonol |
| | | | | | Biosynthesis | | reductase/cinnamoyl-CoA |
| | | | | | | | reductase |
| 414 | Gma.4249.1.S1_at | 0.401 | BF324893 | Secondary Metabolism | | | Methyltransferase |
| 415 | GmaAffx.14141.1.A1_at | 0.401 | BI973009 | No Homology to Known | | | |
| | | | | Proteins | | | |
| 416 | GmaAffx.11496.1.S1_at | 0.4 | BE821691 | Transporter | Inorganic Solute | Putative sulfate | Sulfate/bicarbonate/oxalate |
| | | | | | Cotransporters | transporter family | exchanger SAT-1 and related |
| | | | | | | | transporters (SLC26 family) |
| 417 | GmaAffx.494.1.S1_at | 0.4 | AI938330 | Cell Structure | | | Chitinase |
| 418 | Gma.3065.1.S1_s_at | 0.399 | AW349564 | Unclassified - | | | |
| | | | | Hypothetical Protein | | | |
| | | | | Supported by cDNA | | | |
| 419 | GmaAffx.52526.1.S1_at | 0.399 | BM085509 | Unclassified - | | | Uncharacterized conserved |
| | | | | Hypothetical Protein | | | protein |
| | | | | Supported by cDNA | | | |
| 420 | Gma.17402.1.A1_at | 0.398 | BE611084 | No Homology to Known | | | |
| | | | | Proteins | | | |
| 421 | GmaAffx.37316.1.S1_at | 0.398 | AW733720 | Unclassified - Protein | | | |
| | | | | with Unknown Function | | | |
| 422 | Gma.137.1.S1_at | 0.397 | AF022464.1 | Metabolism | Cytochrome P450 | | Cytochrome P450 CYP2 |
| | | | | | | | subfamily |
| 423 | Gma.17594.2.S1_at | 0.397 | BM093196 | Metabolism | | | Cystathionine beta- |
| | | | | | | | lyases/cystathionine gamma- |
| | | | | | | | synthases |
| 424 | Gma.10873.1.S1_s_at | 0.395 | BQ079465 | No Homology to Known | | | |
| | | | | Proteins | | | |
| 425 | GmaAffx.3369.1.S1_at | 0.395 | BU548983 | Cell Structure | | | |
| 426 | Gma.4728.1.A1_at | 0.394 | BI968944 | Secondary Metabolism | | | NADH:flavin |
| | | | | | | | oxidoreductase/12- |
| | | | | | | | oxophytodienoate reductase |
| 427 | GmaAffx.86212.1.S1_at | 0.394 | BG043474 | Unclassified - | | | |
| | | | | Hypothetical Protein | | | |
| | | | | NOT Supported by | | | |
| | | | | cDNA | | | |
| 428 | GmaAffx.62153.1.S1_at | 0.394 | AW394575 | No Homology to Known | | | |
| | | | | Proteins | | | |
| 429 | Gma.6264.1.S1_at | 0.393 | BQ612558 | Unclassified - | | | |
| | | | | Hypothetical Protein | | | |
| | | | | Supported by cDNA | | | |
| 430 | GmaAffx.37988.1.S1_at | 0.392 | BF324942 | No Homology to Known | | | |
| | | | | Proteins | | | |
| 431 | GmaAffx.72807.1.S1_at | 0.391 | AW831487 | No Homology to Known | | | |
| | | | | Proteins | | | |
| 432 | Gma.16875.1.A1_at | 0.39 | CD406723 | Metabolism | | | Aldo/keto reductase family |
| | | | | | | | proteins |
| 433 | GmaAffx.70659.1.S1_at | 0.39 | AI495240 | No Homology to Known | | | |
| | | | | Proteins | | | |
| 434 | GmaAffx.70005.1.S1_s_at | 0.39 | CA936335 | Secondary Metabolism | | | Iron/ascorbate family |
| | | | | | | | oxidoreductases |
| 435 | GmaAffx.91358.1.S1_x_at | 0.39 | CF807083 | No Homology to Known | | | |
| | | | | Proteins | | | |
| 436 | GmaAffx.32011.1.S1_at | 0.389 | BI701198 | No Homology to Known | | | |
| | | | | Proteins | | | |
| 437 | GmaAffx.80584.1.S1_at | 0.388 | BU083387 | Transcription | Transcription | Basic Helix-Loop- | |
| | | | | | Factor | Helix (bHLH) | |
| 438 | GmaAffx.70548.1.A1_at | 0.388 | CD411723 | No Homology to Known | | | |
| | | | | Proteins | | | |
| 439 | Gma.6033.2.S1_at | 0.387 | AI416728 | Unclassified - | | | |
| | | | | Hypothetical Protein | | | |
| | | | | Supported by cDNA | | | |
| 440 | GmaAffx.93328.1.S1_s_at | 0.387 | CF809053 | Secondary Metabolism | | | |
| 441 | Gma.16359.1.A1_at | 0.387 | BI970359 | Metabolism | | | GPI-alpha- |
| | | | | | | | mannosyltransferase III |
| | | | | | | | (GPI10/PIG-B) involved in |
| | | | | | | | glycosylphosphatidylinositol |
| | | | | | | | anchor biosynthesis |
| 442 | GmaAffx.71038.1.S1_at | 0.386 | BI700350 | Transcription | Transcription | Homeodomain | Transcription factor MEIS1 |
| | | | | | Factor | | and related HOX domain |
| | | | | | | | proteins |
| 443 | GmaAffx.68507.1.S1_at | 0.384 | BI699405 | Transcription | Transcription | Heat shock | Heat shock transcription |
| | | | | | Factor | | factor |
| 444 | GmaAffx.87793.1.S1_at | 0.384 | BI941782 | Unclassified - | | | |
| | | | | Hypothetical Protein | | | |
| | | | | NOT Supported by | | | |
| | | | | cDNA | | | |
| 445 | Gma.5701.3.S1_at | 0.384 | CA852693 | No Homology to Known | | | |
| | | | | Proteins | | | |
| 446 | GmaAffx.48438.1.S1_at | 0.384 | AW350858 | Protein Destination & | | | Molecular chaperone (DnaJ |
| | | | | Storage | | | superfamily) |
| 447 | GmaAffx.15792.1.A1_at | 0.384 | AW101026 | No Homology to Known | | | |
| | | | | Proteins | | | |
| 448 | Gma.3204.1.S1_at | 0.383 | AW509002 | Metabolism | | | Dehydrogenase kinase |
| 449 | Gma.4506.1.S1_at | 0.382 | BQ740943 | Unclassified - | | | |
| | | | | Hypothetical Protein | | | |
| | | | | NOT Supported by | | | |
| | | | | cDNA | | | |
| 450 | GmaAffx.58125.1.A1_at | 0.381 | BE347561 | Transcription | Transcription | Basic Helix-Loop- | |
| | | | | | Factor | Helix (bHLH) | |
| 451 | Gma.17070.1.S1_at | 0.381 | BE822463 | Disease & Defense | | | O-methyltransferase |
| 452 | GmaAffx.74933.1.S1_at | 0.38 | CA935084 | Unclassified - Protein | | | Aspartyl protease |
| | | | | with Unknown Function | | | |
| 453 | GmaAffx.84964.1.S1_at | 0.379 | AW598267 | No Homology to Known | | | |
| | | | | Proteins | | | |
| 454 | GmaAffx.82034.1.S1_at | 0.379 | BM887093 | Transcription | Transcription | Homeodomain | Transcription factor HEX, |
| | | | | | Factor | | contains HOX and HALZ |
| | | | | | | | domains |
| 455 | Gma.4687.1.S1_at | 0.379 | AW309716 | Secondary Metabolism | | | |
| 456 | Gma.13187.1.S1_at | 0.379 | BG839239 | Unclassified - | | | Uncharacterized conserved |
| | | | | Hypothetical Protein | | | protein |
| | | | | Supported by cDNA | | | |
| 457 | Gma.3384.1.S1_at | 0.378 | BE659400 | Unclassified - Protein | | | Acyl-CoA reductase |
| | | | | with Unknown Function | | | |
| 458 | GmaAffx.7838.3.S1_s_at | 0.378 | CA800410 | Metabolism | | | |
| 459 | GmaAffx.40114.1.A1_at | 0.378 | BE661587 | Unclassified - | | | |
| | | | | Hypothetical Protein | | | |
| | | | | Supported by cDNA | | | |
| 460 | GmaAffx.85657.1.S1_s_at | 0.378 | AW185911 | Unclassified - Protein | | | FOG: Kelch repeat |
| | | | | with Unknown Function | | | |
| 461 | Gma.17798.2.S1_at | 0.376 | CD412748 | No Homology to Known | | | |
| | | | | Proteins | | | |
| 462 | Gma.8514.1.S1_at | 0.375 | CA851666 | Protein Destination & | | | Glutathione S-transferase |
| | | | | Storage | | | |
| 463 | Gma.4394.1.S1_a_at | 0.375 | CD409273 | Transcription | Transcription | Homeodomain | Transcription factor HEX, |
| | | | | | Factor | | contains HOX and HALZ |
| | | | | | | | domains |
| 464 | Gma.11269.1.S1_at | 0.374 | CD395046 | Transporter | | | |
| 465 | GmaAffx.71711.1.S1_at | 0.374 | AI988272 | No Homology to Known | | | |
| | | | | Proteins | | | |
| 466 | GmaAffx.8712.1.S1_s_at | 0.373 | AW349107 | Disease & Defense | | | |
| 467 | GmaAffx.90812.1.S1_at | 0.373 | CF806537 | Disease & Defense | | | |
| 468 | GmaAffx.19118.1.S1_at | 0.372 | BM523731 | Unclassified - | | | |
| | | | | Hypothetical Protein | | | |
| | | | | NOT Supported by | | | |
| | | | | cDNA | | | |
| 469 | Gma.398.1.A1_at | 0.372 | AW350679 | Metabolism | | | Homocysteine S- |
| | | | | | | | methyltransferase |
| 470 | Gma.5294.1.S1_at | 0.372 | BE658252 | Unclassified - | | | |
| | | | | Hypothetical Protein | | | |
| | | | | Supported by cDNA | | | |
| 471 | GmaAffx.78163.1.S1_s_at | 0.371 | BM520857 | Signal Transduction | Protein | | Serine/threonine protein |
| | | | | | Phosphatase | | phosphatase |
| 472 | Gma.3450.1.S1_at | 0.37 | BE657895 | Unclassified - | | | |
| | | | | Hypothetical Protein | | | |
| | | | | Supported by cDNA | | | |
| 473 | Gma.9259.1.S1_at | 0.37 | CA782161 | Transporter | | | |
| 474 | GmaAffx.90069.1.S1_s_at | 0.369 | CF805794 | Unclassified - | | | Leucine rich repeat proteins, |
| | | | | Hypothetical Protein | | | some proteins contain F-box |
| | | | | Supported by cDNA | | | |
| 475 | GmaAffx.91037.1.S1_s_at | 0.367 | CF806762 | Secondary Metabolism | | | Methyltransferase |
| 476 | Gma.16910.1.S1_at | 0.367 | BU549219 | Unclassified - Protein | | | |
| | | | | with Unknown Function | | | |
| 477 | GmaAffx.93183.1.S1_s_at | 0.365 | CF808908 | Secondary Metabolism | | | S-adenosylmethionine |
| | | | | | | | decarboxylase |
| 478 | Gma.11166.1.S1_s_at | 0.36 | AW705829 | Metabolism | | | |
| 479 | GmaAffx.81999.1.S1_at | 0.36 | AI416637 | Transcription | Transcription | Homeodomain | Transcription factor MEIS1 |
| | | | | | Factor | | and related HOX domain |
| | | | | | | | proteins |
| 480 | Gma.5725.1.S1_at | 0.36 | BM886367 | Disease & Defense | | | |
| 481 | GmaAffx.64261.1.A1_at | 0.359 | BI969080 | Unclassified - Protein | | | 1,4-benzoquinone reductase- |
| | | | | with Unknown Function | | | like; Trp repressor binding |
| | | | | | | | protein-like/protoplast- |
| | | | | | | | secreted protein |
| 482 | Gma.1951.2.S1_a_at | 0.358 | CA783485 | Protein Destination & | | | Serine carboxypeptidases |
| | | | | Storage | | | |
| 483 | GmaAffx.92127.1.S1_s_at | 0.358 | CF807852 | Transcription | Transcription | AP2/EREBP | |
| | | | | | Factor | | |
| 484 | Gma.3901.1.S1_at | 0.358 | CD394788 | Protein Destination & | Storage Protein | | |
| | | | | Storage | | | |
| 485 | Gma.8240.1.S1_at | 0.357 | BM092761 | Secondary Metabolism | | | Iron/ascorbate family |
| | | | | | | | oxidoreductases |
| 486 | Gma.13845.1.A1_at | 0.355 | CD407676 | Unclassified - | | | |
| | | | | Hypothetical Protein | | | |
| | | | | Supported by cDNA | | | |
| 487 | GmaAffx.1620.2.S1_at | 0.355 | BF324548 | Unclassified - | | | |
| | | | | Hypothetical Protein | | | |
| | | | | NOT Supported by | | | |
| | | | | cDNA | | | |
| 488 | GmaAffx.53845.1.S1_at | 0.354 | BG406552 | Transporter | | | Sulfate/bicarbonate/oxalate |
| | | | | | | | exchanger SAT-1 and related |
| | | | | | | | transporters (SLC26 family) |
| 489 | GmaAffx.92166.1.S1_s_at | 0.353 | CF806313 | Signal Transduction | Unclassified | | Aspartyl protease |
| 490 | Gma.1314.1.S1_at | 0.353 | U63726.1 | Metabolism | | | Gamma-glutamyl hydrolase |
| 491 | GmaAffx.32606.1.S1_at | 0.353 | BM270707 | Unclassified - | | | Aquaporin (major intrinsic |
| | | | | Hypothetical Protein | | | protein family) |
| | | | | NOT Supported by | | | |
| | | | | cDNA | | | |
| 492 | Gma.790.1.A1_s_at | 0.351 | BE820450 | Unclassified - | | | Predicted E3 ubiquitin ligase |
| | | | | Hypothetical Protein | | | |
| | | | | Supported by cDNA | | | |
| 493 | GmaAffx.15471.3.S1_x_at | 0.351 | BE346777 | Transcription | Transcription | MYB | Transcription factor, Myb |
| | | | | | Factor | | superfamily |
| 494 | Gma.7227.2.S1_a_at | 0.349 | AW759183 | Protein Destination & | | | Ubiquitin-protein ligase |
| | | | | Storage | | | |
| 495 | Gma.3578.1.S1_at | 0.349 | AW156522 | Disease & Defense | | | Acyl-CoA synthetase |
| 496 | GmaAffx.47806.1.A1_at | 0.349 | BG508458 | No Homology to Known | | | |
| | | | | Proteins | | | |
| 497 | GmaAffx.75459.1.S1_s_at | 0.348 | BE348034 | Protein Destination & | Storage Protein | | |
| | | | | Storage | | | |
| 498 | GmaAffx.69198.1.S1_at | 0.347 | BM526951 | Unclassified - | | | Leucine rich repeat proteins, |
| | | | | Hypothetical Protein | | | some proteins contain F-box |
| | | | | Supported by cDNA | | | |
| 499 | GmaAffx.88945.1.S1_at | 0.347 | CK605738 | Unclassified - Protein | | | |
| | | | | with Unknown Function | | | |
| 500 | Gma.7454.1.S1_a_at | 0.345 | CD405901 | Transcription | Transcription | AUX/IAA | |
| | | | | | Factor | | |
| 501 | Gma.1326.1.S1_at | 0.345 | CD397515 | Secondary Metabolism | | | |
| 502 | GmaAffx.78768.1.S1_s_at | 0.344 | CA784898 | No Homology to Known | | | |
| | | | | proteins | | | |
| 503 | GmaAffx.69369.1.S1_at | 0.343 | BG511072 | Unclassified - | | | Galactosyltransferases |
| | | | | Hypothetical Protein | | | |
| | | | | Supported by cDNA | | | |
| 504 | GmaAffx.82936.1.S1_at | 0.342 | AI938364 | Unclassified - Protein | | | |
| | | | | with Unknown Function | | | |
| 505 | GmaAffx.58606.1.S1_at | 0.341 | BM526502 | Metabolism | | | |
| 506 | Gma.10892.1.S1_a_at | 0.341 | BG839570 | Metabolism | | | Predicted carbonic |
| | | | | | | | anhydrase involved in |
| | | | | | | | protection against oxidative |
| | | | | | | | damage |
| 507 | Gma.17806.1.S1_at | 0.34 | BE820775 | Transporter | | | H+/oligopeptide symporter |
| 508 | GmaAffx.52114.1.S1_at | 0.339 | CA852143 | Metabolism | | | Heme oxygenase |
| 509 | GmaAffx.36470.1.A1_at | 0.337 | BU550552 | Unclassified - | | | |
| | | | | Hypothetical Protein | | | |
| | | | | Supported by cDNA | | | |
| 510 | GmaAffx.78768.2.S1_at | 0.335 | AI930773 | Unclassified - | | | |
| | | | | Hypothetical Protein | | | |
| | | | | Supported by cDNA | | | |
| 511 | Gma.1263.1.S1_at | 0.334 | AY144180.1 | Metabolism | | | Ca2+/calmodulin-dependent |
| | | | | | | | protein kinase, EF-Hand |
| | | | | | | | protein superfamily |
| 512 | Gma.17688.1.S1_s_at | 0.331 | CA937360 | Transcription | Transcription | Basic Helix-Loop- | |
| | | | | | Factor | Helix (bHLH) | |
| 513 | GmaAffx.11242.1.S1_s_at | 0.329 | AW317461 | Protein Destination & | | | Serine carboxypeptidases |
| | | | | Storage | | | |
| 514 | GmaAffx.86844.1.S1_at | 0.329 | BE822146 | Unclassified - Protein | | | |
| | | | | with Unknown Function | | | |
| 515 | Gma.398.2.S1_at | 0.328 | BU925900 | Metabolism | | | Homocysteine S- |
| | | | | | | | methyltransferase |
| 516 | Gma.221.1.S1_at | 0.328 | U41475.1 | Metabolism | | | Phosphoinositide-specific |
| | | | | | | | phospholipase C |
| 517 | Gma.12987.1.S1_at | 0.327 | CD392747 | Transcription | Transcription | Basic Helix-Loop- | |
| | | | | | Factor | Helix (bHLH) | |
| 518 | GmaAffx.50980.1.S1_s_at | 0.327 | BE823095 | Signal Transduction | Protein | | |
| | | | | | Phosphatase | | |
| 519 | GmaAffx.17892.1.S1_at | 0.326 | AI442822 | Unclassified - | | | |
| | | | | Hypothetical Protein | | | |
| | | | | Supported by cDNA | | | |
| 520 | GmaAffx.33121.1.S1_at | 0.325 | BQ611246 | Unclassified - | | | |
| | | | | Hypothetical Protein | | | |
| | | | | Supported by cDNA | | | |
| 521 | GmaAffx.77832.1.S1_at | 0.323 | BI971626 | Unclassified - | | | |
| | | | | Hypothetical Protein | | | |
| | | | | NOT Supported by | | | |
| | | | | cDNA | | | |
| 522 | GmaAffx.32011.2.S1_at | 0.322 | BM084966 | Transcription | Transcription | Basic Leucine | |
| | | | | | Factor | Zipper (bZ1P) | |
| 523 | Gma.7437.1.S1_at | 0.322 | BU547705 | Unclassified - | | | |
| | | | | Hypothetical Protein | | | |
| | | | | Supported by cDNA | | | |
| 524 | GmaAffx.18113.2.S1_s_at | 0.321 | AW706795 | Unclassified - Protein | Hormone Induced | | |
| | | | | with Unknown Function | | | |
| 525 | Gma.17516.1.S1_at | 0.321 | CD396417 | Transporter | | | Ca2+/H+ antiporter VCX1 |
| | | | | | | | and related proteins |
| 526 | Gma.5889.1.S1_at | 0.32 | BQ610202 | Unclassified - | | | Endoplasmic reticulum |
| | | | | Hypothetical Protein | | | protein EP58, contains |
| | | | | NOT Supported by | | | filamin rod domain and |
| | | | | cDNA | | | KDEL motif |
| 527 | GmaAffx.80188.1.A1_at | 0.317 | AW349832 | No Homology to Known | | | |
| | | | | Proteins | | | |
| 528 | GmaAffx.23292.1.A1_at | 0.317 | BI967795 | No Homology to Known | | | |
| | | | | Proteins | | | |
| 529 | Gma.13030.1.S1_a_at | 0.316 | AI748012 | Protein Destination & | | | |
| | | | | Storage | | | |
| 530 | Gma.12731.2.A1_s_at | 0.315 | CD395848 | Unclassified - | | | Aquaporin (major intrinsic |
| | | | | Hypothetical Protein | | | protein family) |
| | | | | NOT Supported by | | | |
| | | | | cDNA | | | |
| 531 | GmaAffx.91312.1.S1_s_at | 0.314 | CF806002 | Transcription | Transcription | Homeodomain | Transcription factor HEX, |
| | | | | | Factor | | contains HOX and HALZ |
| | | | | | | | domains |
| 532 | Gma.1058.1.S1_at | 0.314 | BE820580 | Metabolism | | | |
| 533 | Gma.9647.1.S1_at | 0.313 | BE820997 | Cell Structure | | | O-methyltransferase |
| 534 | Gma.6033.1.S1_at | 0.312 | BI967767 | Unclassified - | | | |
| | | | | Hypothetical Protein | | | |
| | | | | Supported by cDNA | | | |
| 535 | Gma.17688.1.S1_at | 0.311 | CA937360 | Transcription | Transcription | Basic Helix-Loop- | |
| | | | | | Factor | Helix (bHLH) | |
| 536 | Gma.6129.2.S1_at | 0.31 | AW186006 | Protein Synthesis | | | Translation initiation factor |
| | | | | | | | 5A (eIF-5A) |
| 537 | GmaAffx.46103.1.S1_at | 0.309 | BM091933 | Metabolism | | | Hydroxymethylglutaryl-CoA |
| | | | | | | | lyase |
| 538 | Gma.9365.1.A1_at | 0.309 | CD418433 | No Homology to Known | | | |
| | | | | Proteins | | | |
| 539 | GmaAffx.40923.1.S1_at | 0.308 | BG653848 | Disease & Defense | | | Selenium-binding protein |
| 540 | Gma.13273.1.A1_at | 0.306 | CD397107 | Unclassified - | | | |
| | | | | Hypothetical Protein | | | |
| | | | | Supported by cDNA | | | |
| 541 | GmaAffx.64967.1.S1_at | 0.306 | BU546115 | Secondary Metabolism | | | Iron/ascorbate family |
| | | | | | | | oxidoreductases |
| 542 | Gma.10969.2.S1_at | 0.302 | CD402329 | Unclassified - | | | Predicted lipase |
| | | | | Hypothetical Protein | | | |
| | | | | NOT Supported by | | | |
| | | | | cDNA | | | |
| 543 | GmaAffx.17918.1.S1_at | 0.301 | BG157024 | No Homology to Known | | | |
| | | | | Proteins | | | |
| 544 | GmaAffx.64807.1.S1_at | 0.301 | AW201972 | No Homology to Known | | | |
| | | | | Proteins | | | |
| 545 | GmaAffx.87793.2.S1_at | 0.301 | BQ740671 | Signal Transduction | Protein Kinase | | Glycogen synthase kinase-3 |
| 546 | Gma.11166.1.S1_x_at | 0.3 | AW705829 | Metabolism | | | |
| 547 | GmaAffx.45036.1.S1_at | 0.3 | BF595470 | No Homology to Known | | | |
| | | | | Proteins | | | |
| 548 | Gma.11205.1.S1_a_at | 0.299 | BF066553 | Metabolism | | | Transketolase |
| 549 | Gma.821.1.S1_at | 0.299 | AF243371.1 | Metabolism | | | Glutathione S-transferase |
| 550 | Gma.1619.1.S1_at | 0.298 | AW472195 | Unclassified - | | | |
| | | | | Hypothetical Protein | | | |
| | | | | Supported by cDNA | | | |
| 551 | Gma.1773.1.A1_at | 0.298 | CD404050 | Transcription | Transcription | Basic Helix-Loop- | |
| | | | | | Factor | Helix (bHLH) | |
| 552 | Gma.5468.2.S1_a_at | 0.298 | BE802025 | Metabolism | | | Beta-galactosidase |
| 553 | GmaAffx.34798.1.S1_at | 0.297 | BM143105 | Unclassified - | | | |
| | | | | Hypothetical Protein | | | |
| | | | | Supported by cDNA | | | |
| 554 | GmaAffx.89697.1.S1_s_at | 0.296 | CK606333 | Disease & Defense | | | |
| 555 | GmaAffx.81692.1.S1_at | 0.294 | CA937041 | No Homology to Known | | | |
| | | | | Proteins | | | |
| 556 | Gma.4990.1.A1_at | 0.294 | BI315863 | Unclassified - | | | Uncharacterized conserved |
| | | | | Hypothetical Protein | | | protein |
| | | | | Supported by cDNA | | | |
| 557 | GmaAffx.6790.1.S1_at | 0.294 | AI856683 | Disease & Defense | | | Soluble epoxide hydrolase |
| 558 | GmaAffx.83715.1.S1_at | 0.294 | BI785710 | Transporter | | | Glutaredoxin-related protein |
| 559 | GmaAffx.91428.1.A1_at | 0.293 | CF807137 | Metabolism | | | |
| 560 | GmaAffx.487.1.S1_at | 0.293 | BG511495 | Transporter | | | Predicted transporter (major |
| | | | | | | | facilitator superfamily) |
| 561 | Gma.5586.2.S1_at | 0.293 | CD398384 | Transcription | Transcription | Unclassified | CCAAT-binding factor, |
| | | | | | Factor | | subunit B (HAP2) |
| 562 | GmaAffx.86020.1.S1_at | 0.293 | BI701699 | Signal Transduction | Protein Kinase | AtCIPKs | Serine/threonine protein |
| | | | | | | | kinase |
| 563 | GmaAffx.24451.1.A1_at | 0.292 | BU550405 | Metabolism | Cytochrome P450 | CYP90C | Cytochrome P450 |
| | | | | | | | CYP4/CYP19/CYP26 |
| | | | | | | | subfamilies |
| 564 | Gma.1951.1.S1_at | 0.292 | BQ079657 | Protein Destination & | | | Serine carboxypeptidases |
| | | | | Storage | | | |
| 565 | Gma.1715.1.S1_at | 0.29 | BE191618 | Metabolism | | | |
| 566 | Gma.1573.2.S1_a_at | 0.286 | BI974098 | Metabolism | | | |
| 567 | GmaAffx.60673.1.S1_at | 0.286 | BU080770 | Unclassified - | | | |
| | | | | Hypothetical Protein | | | |
| | | | | NOT Supported by | | | |
| | | | | cDNA | | | |
| 568 | Gma.11873.1.S1_at | 0.283 | CA819977 | Unclassified - Protein | | | |
| | | | | with Unknown Function | | | |
| 569 | Gma.3617.1.S1_at | 0.282 | BE659297 | Cell Growth & Division | | | |
| 570 | GmaAffx.74438.1.S1_at | 0.282 | BE440325 | Unclassified - | | | |
| | | | | Hypothetical Protein | | | |
| | | | | NOT Supported by | | | |
| | | | | cDNA | | | |
| 571 | GmaAffx.18113.2.S1_x_at | 0.282 | AW706795 | Unclassified - Protein | Hormone Induced | | |
| | | | | with Unknown Function | | | |
| 572 | GmaAffx.6.1.S1_at | 0.276 | AW348562 | No Homology to Known | | | |
| | | | | Proteins | | | |
| 573 | Gma.8058.1.A1_at | 0.276 | BM271479 | Signal Transduction | Protein | | Serine/threonine protein |
| | | | | | Phosphatase | | phosphatase |
| 574 | Gma.1768.1.S1_s_at | 0.276 | BE820777 | Unclassified - | | | |
| | | | | Hypothetical Protein | | | |
| | | | | Supported by cDNA | | | |
| 575 | Gma.5701.2.S1_at | 0.275 | BI701059 | Unclassified - | | | |
| | | | | Hypothetical Protein | | | |
| | | | | Supported by cDNA | | | |
| 576 | GmaAffx.90395.1.S1_s_at | 0.275 | CF806120 | Unclassified - Protein | | | |
| | | | | with Unknown Function | | | |
| 577 | GmaAffx.17892.2.S1_at | 0.273 | BQ628677 | No Homology to Known | | | |
| | | | | Proteins | | | |
| 578 | GmaAffx.93591.1.S1_s_at | 0.27 | CF809316 | Metabolism | | | |
| 579 | Gma.10520.1.A1_at | 0.27 | CD398227 | Unclassified - | | | |
| | | | | Hypothetical Protein | | | |
| | | | | Supported by cDNA | | | |
| 580 | Gma.11026.2.S1_s_at | 0.27 | BQ080041 | Disease & Defense | | | |
| 581 | GmaAffx.84424.1.S1_at | 0.27 | AW311543 | Metabolism | | | Alcohol dehydrogenase, |
| | | | | | | | class V |
| 582 | Gma.1394.2.S1_a_at | 0.269 | BI470370 | Metabolism | | | |
| 583 | Gma.6192.1.S1_s_at | 0.269 | AW396773 | Metabolism | | | |
| 584 | GmaAffx.40114.1.S1_at | 0.267 | BI973325 | Unclassified - | | | |
| | | | | Hypothetical Protein | | | |
| | | | | Supported by cDNA | | | |
| 585 | Gma.6763.1.A1_at | 0.266 | BU544794 | Energy | | | Ferric reductase, |
| | | | | | | | NADH/NADPH oxidase and |
| | | | | | | | related proteins |
| 586 | Gma.10581.1.S1_at | 0.262 | AW396439 | Unclassified - | | | Predicted haloacid |
| | | | | Hypothetical Protein | | | dehalogenase-like hydrolase |
| | | | | Supported by cDNA | | | |
| 587 | Gma.1394.1.S1_at | 0.261 | BG839300 | Metabolism | | | |
| 588 | Gma.15487.1.S1_at | 0.259 | D63781.1 | Disease & Defense | | | Soluble epoxide hydrolase |
| 589 | GmaAffx.93592.1.S1_s_at | 0.259 | CF809317 | Unclassified - | | | |
| | | | | Hypothetical Protein | | | |
| | | | | Supported by cDNA | | | |
| 590 | Gma.4359.1.S1_at | 0.258 | AI794712 | Energy | | | Thioredoxin |
| 591 | GmaAffx.1620.1.S1_at | 0.257 | AW424105 | Unclassified - | | | |
| | | | | Hypothetical Protein | | | |
| | | | | NOT Supported by | | | |
| | | | | cDNA | | | |
| 592 | GmaAffx.1620.3.S1_s_at | 0.255 | AW132226 | No Homology to Known | | | |
| | | | | Proteins | | | |
| 593 | Gma.1980.1.A1_at | 0.255 | BM520466 | Signal Transduction | Protein Kinase | AtCIPKs | Serine/threonine protein |
| | | | | | | | kinase |
| 594 | GmaAffx.53324.1.A1_at | 0.254 | AW309251 | Transcription | Transcription | Basic Helix-Loop- | |
| | | | | | Factor | Helix (bHLH) | |
| 595 | Gma.3118.1.S1_s_at | 0.254 | BQ786048 | Disease & Defense | | | Predicted metallothionein |
| 596 | GmaAffx.77203.1.S1_at | 0.253 | BI970687 | Unclassified - | | | |
| | | | | Hypothetical Protein | | | |
| | | | | Supported by cDNA | | | |
| 597 | GmaAffx.52826.3.S1_at | 0.252 | BF596503 | Signal Transduction | Protein | | Serine/threonine protein |
| | | | | | Phosphatase | | phosphatase |
| 598 | GmaAffx.57432.1.S1_at | 0.25 | BE657443 | Unclassified - | | | |
| | | | | Hypothetical Protein | | | |
| | | | | NOT Supported by | | | |
| | | | | cDNA | | | |
| 599 | Gma.1394.2.S1_at | 0.249 | BI470370 | Metabolism | | | |
| 600 | Gma.15863.1.S1_s_at | 0.248 | CD413163 | Metabolism | | | |
| 601 | GmaAffx.93342.1.S1_s_at | 0.247 | CF809067 | Disease & Defense | | | Glutathione peroxidase |
| 602 | Gma.15093.1.S1_at | 0.247 | CD400374 | Protein Destination & | | | Glutathione S-transferase |
| | | | | Storage | | | |
| 603 | GmaAffx.91514.1.S1_at | 0.246 | CF807239 | No Homology to Known | | | |
| | | | | Proteins | | | |
| 604 | GmaAffx.90600.1.S1_at | 0.245 | CF806325 | Metabolism | | | |
| 605 | Gma.13672.1.A1_at | 0.242 | CD403249 | No Homology to Known | | | |
| | | | | Proteins | | | |
| 606 | Gma.17985.1.S1_at | 0.239 | CD416450 | Metabolism | | | |
| 607 | GmaAffx.90652.1.S1_at | 0.238 | CF806377 | Unclassified - | | | Predicted |
| | | | | Hypothetical Protein | | | hydrolase/acyltransferase |
| | | | | Supported by cDNA | | | (alpha/beta hydrolase |
| | | | | | | | superfamily) |
| 608 | Gma.8674.3.A1_at | 0.238 | BI469914 | Transporter | | | Predicted transporter (major |
| | | | | | | | facilitator superfamily) |
| 609 | Gma.13013.2.S1_a_at | 0.237 | AW457857 | Unclassified - | | | |
| | | | | Hypothetical Protein | | | |
| | | | | Supported by cDNA | | | |
| 610 | GmaAffx.89772.8.S1_at | 0.237 | CK605778 | Metabolism | | | |
| 611 | Gma.6684.1.S1_at | 0.236 | AW459859 | Signal Transduction | Unclassified | | Lanthionine synthetase C- |
| | | | | | | | like protein 1 |
| 612 | Gma.16500.1.S1_at | 0.235 | BE059054 | Metabolism | | | |
| 613 | GmaAffx.15196.1.S1_at | 0.234 | BU549436 | Metabolism | | | Long-chain acyl-CoA |
| | | | | | | | synthetases (AMP-forming) |
| 614 | GmaAffx.58712.1.S1_at | 0.233 | BF325034 | Transporter | | | Transporter, ABC |
| | | | | | | | superfamily (Breast cancer |
| | | | | | | | resistance protein) |
| 615 | GmaAffx.47288.1.S1_at | 0.233 | BU764979 | Unclassified - | | | GTPase Rab18, small G |
| | | | | Hypothetical Protein | | | protein superfamily |
| | | | | Supported by cDNA | | | |
| 616 | GmaAffx.40015.1.S1_at | 0.23 | BE609673 | No Homology to Known | | | |
| | | | | Proteins | | | |
| 617 | Gma.2086.1.S1_at | 0.228 | AF035671.1 | Metabolism | Amino Acid | | Arginase family protein |
| | | | | | Biosynthesis/Metabolism | | |
| 618 | GmaAffx.15983.2.S1_s_at | 0.228 | AW279540 | Signal Transduction | Protein | | Serine/threonine protein |
| | | | | | Phosphatase | | phosphatase |
| 619 | GmaAffx.50980.1.S1_at | 0.227 | BE823095 | Signal Transduction | Protein | | |
| | | | | | Phosphatase | | |
| 620 | Gma.10827.3.S1_at | 0.227 | AW758966 | Secondary Metabolism | | | Iron/ascorbate family |
| | | | | | | | oxidoreductases |
| 621 | Gma.2252.1.S1_at | 0.227 | CD406598 | Unclassified - | | | |
| | | | | Hypothetical Protein | | | |
| | | | | Supported by cDNA | | | |
| 622 | GmaAffx.50980.1.S1_x_at | 0.225 | BE823095 | Signal Transduction | Protein | | |
| | | | | | Phosphatase | | |
| 623 | Gma.1527.2.S1_at | 0.223 | BE658017 | Secondary Metabolism | | | Flavonol |
| | | | | | | | reductase/cinnamoyl-CoA |
| | | | | | | | reductase |
| 624 | Gma.7006.1.S1_at | 0.223 | AF516880.1 | Cell Structure | | | |
| 625 | GmaAffx.15983.2.S1_at | 0.221 | AW279540 | Signal Transduction | Protein | | Serine/threonine protein |
| | | | | | Phosphatase | | phosphatase |
| 626 | Gma.3420.1.S1_at | 0.219 | CD406082 | Unclassified - | | | |
| | | | | Hypothetical Protein | | | |
| | | | | Supported by cDNA | | | |
| 627 | Gma.4252.1.S1_at | 0.216 | BE821208 | Metabolism | | | Glutamate decarboxylase |
| | | | | | | | and related proteins |
| 628 | GmaAffx.66215.1.S1_at | 0.215 | AI443109 | Signal Transduction | Unclassified | | Lanthionine synthetase C- |
| | | | | | | | like protein 1 |
| 629 | Gma.1979.1.S1_a_at | 0.213 | CD403280 | Unclassified - Protein | | | Multitransmembrane protein |
| | | | | with Unknown Function | | | |
| 630 | Gma.7824.1.S1_at | 0.212 | BU550492 | Metabolism | | | Kynurenine 3- |
| | | | | | | | monooxygenase and related |
| | | | | | | | flavoprotein |
| | | | | | | | monooxygenases |
| 631 | GmaAffx.15862.1.S1_at | 0.211 | BM092634 | Cell Structure | | | |
| 632 | GmaAffx.47788.1.A1_at | 0.21 | CD408318 | Metabolism | | | Aldehyde dehydrogenase |
| 633 | Gma.17985.2.S1_at | 0.209 | BE802263 | Metabolism | | | |
| 634 | Gma.968.1.S1_at | 0.209 | D28876.1 | Protein Destination & | | | Asparaginyl peptidases |
| | | | | Storage | | | |
| 635 | GmaAffx.38555.2.S1_at | 0.208 | BI425873 | No Homology to Known | | | |
| | | | | Proteins | | | |
| 636 | GmaAffx.79283.1.S1_at | 0.208 | CA819076 | Unclassified - | | | |
| | | | | Hypothetical Protein | | | |
| | | | | NOT Supported by | | | |
| | | | | cDNA | | | |
| 637 | Gma.7580.1.S1_at | 0.208 | BE057763 | Unclassified - | | | |
| | | | | Hypothetical Protein | | | |
| | | | | Supported by cDNA | | | |
| 638 | Gma.17921.2.S1_s_at | 0.208 | BM085020 | Metabolism | Sucrose | | Glycosyltransferase |
| | | | | | Biosynthesis | | |
| 639 | GmaAffx.90600.1.S1_s_at | 0.207 | CF806325 | Metabolism | | | |
| 640 | GmaAffx.92964.1.S1_at | 0.206 | CF808689 | No Homology to Known | | | |
| | | | | Proteins | | | |
| 641 | GmaAffx.24413.1.A1_at | 0.205 | BU548368 | Metabolism | | | 5′-AMP-activated protein |
| | | | | | | | kinase, gamma subunit |
| 642 | Gma.11672.1.S1_at | 0.205 | BF425929 | Unclassified - | | | |
| | | | | Hypothetical Protein | | | |
| | | | | Supported by cDNA | | | |
| 643 | Gma.6333.2.S1_at | 0.202 | CD396361 | No Homology to Known | | | |
| | | | | Proteins | | | |
| 644 | GmaAffx.93592.1.S1_x_at | 0.202 | CF809317 | Unclassified - | | | |
| | | | | Hypothetical Protein | | | |
| | | | | Supported by cDNA | | | |
| 645 | Gma.5469.1.S1_at | 0.202 | AW349200 | No Homology to Known | | | |
| | | | | Proteins | | | |
| 646 | Gma.1426.1.A1_at | 0.202 | AI443364 | Metabolism | | | |
| 647 | GmaAffx.11172.1.A1_at | 0.201 | CD404556 | No Homology to Known | | | |
| | | | | Proteins | | | |
| 648 | Gma.13150.1.A1_at | 0.2 | CD411577 | Unclassified - Protein | Hormone Induced | | |
| | | | | with Unknown Function | | | |
| 649 | Gma.13340.1.S1_at | 0.199 | BM523855 | Metabolism | | | UDP-glucuronosyl and UDP- |
| | | | | | | | glucosyl transferase |
| 650 | Gma.177.1.S1_at | 0.196 | AF004806.1 | Protein Destination & | Storage Protein | | Reticulon |
| | | | | Storage | | | |
| 651 | GmaAffx.87417.1.S1_at | 0.195 | BM568072 | Metabolism | | | |
| 652 | GmaAffx.64794.1.S1_at | 0.192 | CA801749 | Unclassified - | | | |
| | | | | Hypothetical Protein | | | |
| | | | | Supported by cDNA | | | |
| 653 | Gma.12068.1.A1_at | 0.192 | BU547739 | Transporter | | | Transporter, ABC |
| | | | | | | | superfamily (Breast cancer |
| | | | | | | | resistance protein) |
| 654 | Gma.6921.1.S1_at | 0.188 | BE823209 | Signal Transduction | Unclassified | | Phosphatidylethanolamine |
| | | | | | | | binding protein |
| 655 | Gma.12227.1.S1_at | 0.186 | BG511671 | Unclassified - | | | |
| | | | | Hypothetical Protein | | | |
| | | | | NOT Supported by | | | |
| | | | | cDNA | | | |
| 656 | GmaAffx.12665.1.A1_at | 0.186 | BE822073 | Metabolism | | | |
| 657 | Gma.6192.1.S1_at | 0.183 | AW396773 | Metabolism | | | |
| 658 | GmaAffx.45957.2.S1_at | 0.181 | BI974135 | No Homology to Known | | | |
| | | | | Proteins | | | |
| 659 | GmaAffx.93220.1.S1_at | 0.18 | CF808945 | No Homology to Known | | | |
| | | | | Proteins | | | |
| 660 | GmaAffx.51466.1.A1_at | 0.179 | CD406245 | Metabolism | | | UDP-glucuronosyl and UDP- |
| | | | | | | | glucosyl transferase |
| 661 | GmaAffx.4968.1.S1_at | 0.178 | BI967691 | Unclassified - | | | |
| | | | | Hypothetical Protein | | | |
| | | | | Supported by cDNA | | | |
| 662 | Gma.13062.1.S1_at | 0.176 | CD394315 | Metabolism | | | |
| 663 | GmaAffx.89772.8.S1_x_at | 0.174 | CK605778 | Metabolism | | | |
| 664 | Gma.1495.1.S1_at | 0.174 | BI973545 | No Homology to Known | | | |
| | | | | Proteins | | | |
| 665 | GmaAffx.48718.1.S1_at | 0.173 | BM528538 | No Homology to Known | | | |
| | | | | Proteins | | | |
| 666 | Gma.2382.1.S1_x_at | 0.171 | BG789835 | Metabolism | | | |
| 667 | Gma.5331.1.S1_a_at | 0.167 | BI892702 | Transcription | Transcription | NAC Domain | |
| | | | | | Factor | | |
| 668 | Gma.2540.2.S1_s_at | 0.165 | BI787366 | No Homology to Known | | | |
| | | | | Proteins | | | |
| 669 | GmaAffx.92167.1.A1_s_at | 0.164 | CF807892 | Unclassified - | | | |
| | | | | Hypothetical Protein | | | |
| | | | | NOT Supported by | | | |
| | | | | cDNA | | | |
| 670 | GmaAffx.31862.1.S1_at | 0.163 | BE822501 | Unclassified - | | | |
| | | | | Hypothetical Protein | | | |
| | | | | Supported by cDNA | | | |
| 671 | Gma.8022.1.S1_at | 0.161 | BQ628998 | Disease & Defense | | | Soluble epoxide hydrolase |
| 672 | Gma.15.1.S1_at | 0.161 | U66316.1 | Unclassified - Protein | | | |
| | | | | with Unknown Function | | | |
| 673 | Gma.1527.1.S1_x_at | 0.16 | BI968185 | Secondary Metabolism | | | Flavonol |
| | | | | | | | reductase/cinnamoyl-CoA |
| | | | | | | | reductase |
| 674 | Gma.10615.1.A1_at | 0.156 | BU545626 | Transcription | Transcription | Basic Helix-Loop- | |
| | | | | | Factor | Helix (bHLH) | |
| 675 | GmaAffx.93116.1.S1_at | 0.15 | CF806290 | Disease & Defense | | | |
| 676 | GmaAffx.71308.1.S1_at | 0.15 | BF595112 | Transcription | Transcription | Heat Shock | Heat shock transcription |
| | | | | | Factor | | factor |
| 677 | Gma.1527.1.S1_a_at | 0.148 | BI968185 | Secondary Metabolism | | | Flavonol |
| | | | | | | | reductase/cinnamoyl-CoA |
| | | | | | | | reductase |
| 678 | GmaAffx.80393.1.A1_at | 0.144 | BE658680 | No Homology to Known | | | |
| | | | | Proteins | | | |
| 679 | Gma.12094.1.A1_at | 0.144 | CD395088 | Unclassified - | | | |
| | | | | Hypothetical Protein | | | |
| | | | | Supported by cDNA | | | |
| 680 | Gma.857.1.S1_at | 0.143 | BE822264 | No Homology to Known | | | |
| | | | | Proteins | | | |
| 681 | Gma.16477.1.S1_at | 0.142 | BE824134 | No Homology to Known | | | |
| | | | | Proteins | | | |
| 682 | Gma.4837.1.S1_at | 0.14 | BI969343 | Transporter | | | H+/oligopeptide symporter |
| 683 | Gma.728.1.A1_at | 0.139 | BE658779 | No Homology to Known | | | |
| | | | | Proteins | | | |
| 684 | Gma.11026.1.S1_at | 0.139 | AW756869 | Disease & Defense | | | |
| 685 | Gma.5331.1.S1_at | 0.131 | BI892702 | Transcription | Transcription | NAC Domain | |
| | | | | | Factor | | |
| 686 | Gma.16566.1.S1_at | 0.131 | CD400974 | Metabolism | | | Fumarase |
| 687 | Gma.13035.1.S1_at | 0.13 | CD396536 | No Homology to Known | | | |
| | | | | Proteins | | | |
| 688 | GmaAffx.57432.2.S1_at | 0.13 | CA819434 | No Homology to Known | | | |
| | | | | Proteins | | | |
| 689 | GmaAffx.89624.1.A1_s_at | 0.124 | CK606100 | Transcription | Transcription | Basic Leucine | |
| | | | | | Factor | Zipper (bZIP) | |
| 690 | Gma.10999.1.S1_at | 0.124 | CA850772 | Disease & Defense | | | |
| 691 | GmaAffx.91338.1.S1_at | 0.123 | CF807063 | Unclassified - | | | Predicted haloacid |
| | | | | Hypothetical Protein | | | dehalogenase-like hydrolase |
| | | | | Supported by cDNA | | | |
| 692 | GmaAffx.71222.1.S1_at | 0.121 | AW460050 | Metabolism | | | 5′-AMP-activated protein |
| | | | | | | | kinase, gamma subunit |
| 693 | GmaAffx.73698.1.S1_at | 0.118 | BU964780 | Metabolism | | | 5′-AMP-activated protein |
| | | | | | | | kinase, gamma subunit |
| 694 | GmaAffx.88546.1.S1_at | 0.114 | BE801134 | Metabolism | | | |
| 695 | GmaAffx.89407.1.A1_s_at | 0.11 | CK606273 | Transporter | | | |
| 696 | Gma.12684.1.S1_at | 0.107 | CD394773 | Transcription | Transcription | Zinc Finger | |
| | | | | | Factor | | |
| 697 | Gma.11993.1.S1_at | 0.107 | BU551348 | Transporter | | | Transporter, ABC |
| | | | | | | | superfamily (Breast cancer |
| | | | | | | | resistance protein) |
| 698 | Gma.2382.1.S1_s_at | 0.107 | BG789835 | Metabolism | | | |
| 699 | GmaAffx.71308.2.A1_at | 0.105 | AW569888 | No Homology to Known | | | |
| | | | | Proteins | | | |
| 700 | Gma.1649.1.A1_at | 0.104 | BE659535 | No Homology to Known | | | |
| | | | | Proteins | | | |
| 701 | GmaAffx.26908.1.S1_at | 0.0998 | BF425834 | Secondary Metabolism | | | Beta, beta-carotene 15,15′- |
| | | | | | | | dioxygenase and related |
| | | | | | | | enzymes |
| 702 | GmaAffx.72198.1.A1_at | 0.099 | BE823481 | Metabolism | | | UDP-glucuronosyl and UDP- |
| | | | | | | | glucosyl transferase |
| 703 | GmaAffx.66301.1.S1_at | 0.0974 | BG510223 | Secondary Metabolism | Ethylene | | Iron/ascorbate family |
| | | | | | Biosynthesis | | oxidoreductases |
| 704 | GmaAffx.30792.1.A1_at | 0.0973 | BU544815 | Unclassified - | | | |
| | | | | Hypothetical Protein | | | |
| | | | | Supported by cDNA | | | |
| 705 | GmaAffx.11049.1.S1_at | 0.0954 | BU545140 | Unclassified - Protein | | | Phosphatidylethanolamine |
| | | | | with Unknown Function | | | binding protein |
| 706 | GmaAffx.3369.2.A1_at | 0.0914 | BU550316 | Cell Structure | | | |
| 707 | GmaAffx.91479.1.S1_s_at | 0.0865 | CF807204 | No Homology to Known | | | |
| | | | | Proteins | | | |
| 708 | GmaAffx.93212.1.S1_s_at | 0.0863 | CF807524 | Transporter | | | |
| 709 | Gma.8530.1.S1_at | 0.086 | AF109720.1 | Protein Destination & | Storage Protein | | |
| | | | | Storage | | | |
| 710 | Gma.10896.3.A1_at | 0.0833 | BI316837 | Metabolism | | | Aldehyde dehydrogenase |
| 711 | GmaAffx.42732.1.S1_at | 0.0824 | BU765857 | Protein Destination & | Ubiquitin | | FOG: Predicted E3 ubiquitin |
| | | | | Storage | Degradation | | ligase |
| 712 | Gma.12393.3.S1_at | 0.0822 | BM085488 | Secondary Metabolism | | | Beta, beta-carotene 15,15′- |
| | | | | | | | dioxygenase and related |
| | | | | | | | enzymes |
| 713 | Gma.7831.1.S1_s_at | 0.0815 | CD393602 | Transporter | | | |
| 714 | Gma.15534.1.S1_at | 0.0813 | U50081.1 | Metabolism | | | |
| 715 | GmaAffx.10582.1.S1_at | 0.0788 | BM523687 | Transcription | Transcription | CCAAT | CCAAT-binding factor, |
| | | | | | Factor | | subunit A (HAP3) |
| 716 | GmaAffx.81851.1.S1_at | 0.0772 | AW706751 | Unclassified - | | | |
| | | | | Hypothetical Protein | | | |
| | | | | Supported by cDNA | | | |
| 717 | GmaAffx.26868.1.S1_at | 0.0752 | BF325443 | Unclassified - | | | Tomosyn and related |
| | | | | Hypothetical Protein | | | SNARE-interacting proteins |
| | | | | NOT Supported by | | | |
| | | | | cDNA | | | |
| 718 | GmaAffx.295.1.A1_s_at | 0.0741 | BG653267 | Protein Destination & | | | |
| | | | | Storage | | | |
| 719 | GmaAffx.12933.1.S1_at | 0.0738 | AW395787 | Transcription | Transcription | CCAAT | CCAAT-binding factor, |
| | | | | | Factor | | subunit B (HAP2) |
| 720 | Gma.2986.1.S1_at | 0.0737 | BM271324 | Metabolism | | | 5′-AMP-activated protein |
| | | | | | | | kinase, gamma subunit |
| 721 | GmaAffx.27331.1.S1_at | 0.0729 | BM520297 | Unclassified - | | | |
| | | | | Hypothetical Protein | | | |
| | | | | NOT Supported by | | | |
| | | | | cDNA | | | |
| 722 | GmaAffx.82263.1.S1_s_at | 0.068 | AW568454 | No Homology to Known | | | |
| | | | | Proteins | | | |
| 723 | GmaAffx.76276.1.S1_at | 0.0658 | BE331452 | Transporter | | | Copper chaperone |
| 724 | GmaAffx.81362.1.S1_at | 0.0651 | BF324988 | Cell Structure | | | |
| 725 | Gma.17326.1.A1_at | 0.0634 | AW568254 | Unclassified - | | | Permease of the major |
| | | | | Hypothetical Protein | | | facilitator superfamily |
| | | | | NOT Supported by | | | |
| | | | | cDNA | | | |
| 726 | Gma.8187.1.S1_a_at | 0.0628 | BU549133 | Signal Transduction | Protein | | Serine/threonine protein |
| | | | | | Phosphatase | | phosphatase |
| 727 | GmaAffx.9911.1.S1_at | 0.0603 | BU547808 | Unclassified - | | | Tomosyn and related |
| | | | | Hypothetical Protein | | | SNARE-interacting proteins |
| | | | | NOT Supported by | | | |
| | | | | cDNA | | | |
| 728 | Gma.1061.1.A1_at | 0.0594 | CD408183 | Metabolism | | | |
| 729 | Gma.13854.1.S1_at | 0.0534 | CD408166 | Metabolism | | | |
| 730 | Gma.8318.1.A1_at | 0.0533 | BU550469 | No Homology to Known | | | |
| | | | | Proteins | | | |
| 731 | GmaAffx.93212.1.S1_at | 0.0532 | CF807524 | Transporter | | | |
| 732 | Gma.5929.1.S1_at | 0.0517 | BQ610446 | No Homology to Known | | | |
| | | | | Proteins | | | |
| 733 | Gma.176.1.S1_s_at | 0.0488 | AF004810.1 | Protein Destination & | Storage Protein | | |
| | | | | Storage | | | |
| 734 | GmaAffx.93212.1.S1_x_at | 0.0475 | CF807524 | Transporter | | | |
| 735 | Gma.13296.3.S1_at | 0.0456 | CD418313 | Transporter | | | |
| 736 | GmaAffx.57970.1.S1_at | 0.0435 | BM886862 | Transcription | Transcription | NAC Domain | |
| | | | | | Factor | | |
| 737 | GmaAffx.75697.1.S1_s_at | 0.0435 | BU090518 | Unclassified - | | | |
| | | | | Hypothetical Protein | | | |
| | | | | Supported by cDNA | | | |
| 738 | GmaAffx.63464.1.S1_at | 0.0409 | BG156739 | No Homology to Known | | | |
| | | | | Proteins | | | |
| 739 | Gma.3774.2.S1_at | 0.0343 | CD393365 | Energy | | | Thioredoxin, nucleoredoxin |
| | | | | | | | and related proteins |
| 740 | Gma.5162.1.A1_at | 0.0327 | BI971156 | Signal Transduction | Protein Kinase | | |
| 741 | Gma.10830.1.S1_at | 0.0323 | BE820822 | Unclassified - | | | |
| | | | | Hypothetical Protein | | | |
| | | | | Supported by cDNA | | | |
| 742 | GmaAffx.10204.1.S1_at | 0.0297 | BQ298213 | Unclassified - | | | |
| | | | | Hypothetical Protein | | | |
| | | | | Supported by cDNA | | | |
| 743 | Gma.10904.1.S1_at | 0.0244 | CD408184 | Protein Destination & | | | |
| | | | | Storage | | | |
| 744 | Gma.3774.1.S1_at | 0.0206 | BU550698 | Energy | | | Thioredoxin, nucleoredoxin |
| | | | | | | | and related proteins |
| 745 | Gma.3774.2.S1_a_at | 0.0202 | CD393365 | Energy | | | Thioredoxin, nucleoredoxin |
| | | | | | | | and related proteins |
| 746 | Gma.9947.1.S1_at | 0.0195 | BF324269 | Unclassified - | | | |
| | | | | Hypothetical Protein | | | |
| | | | | Supported by cDNA | | | |
| 747 | Gma.2912.1.S1_s_at | 0.0193 | AF117884.1 | Protein Destination & | Storage Protein | | Uncharacterized conserved |
| | | | | Storage | | | protein |
| 748 | Gma.3000.1.S1_at | 0.0192 | BE657172 | No Homology to Known | | | |
| | | | | Proteins | | | |
| 749 | Gma.10904.1.S1_s_at | 0.0156 | CD408184 | Protein Destination & | | | |
| | | | | Storage | | | |
| 750 | GmaAffx.1785.1.S1_at | 0.0156 | BF325331 | Transcription | Transcription | Homeodomain | Transcription factor HEX, |
| | | | | | Factor | | contains HOX and HALZ |
| | | | | | | | domains |
| 751 | Gma.2912.1.S1_at | 0.0134 | AF117884.1 | Protein Destination & | Storage Protein | | Uncharacterized conserved |
| | | | | Storage | | | protein |
| 752 | Gma.3000.2.S1_a_at | 0.0111 | BM270844 | No Homology to Known | | | |
| | | | | Proteins | | | |
| 753 | GmaAffx.68621.1.A1_at | 0.00817 | BI893889 | Unclassified - Protein | Stress Induced | | |
| | | | | with Unknown Function | | | |

TABLE 3

| | | | Soybean_Gen | |
|---|---|---|---|---|
| ID | Gene Name | Fold Change | Bank_Acc | Functional Category |
| 1 | Gma.4058.1.S1_at | 29.77 | AW310338 | Transporter |
| 2 | GmaAffx.19116.1.S1_at | 18.56 | BI968419 | Signal Transduction |
| 3 | Gma.10921.1.S1_s_at | 18.02 | BQ611250 | No Homology to Known Proteins |
| 4 | Gma.4071.1.S1_at | 12.74 | BG651692 | Unclassified - Hypothetical |
| | | | | Protein Supported by cDNA |
| 5 | GmaAffx.32345.1.A1_s_at | 12.69 | BI970277 | Cell Structure |
| 6 | GmaAffx.89075.1.S1_s_at | 10.01 | CK606454 | No Homology to Known Proteins |
| 7 | GmaAffx.93260.1.S1_at | 9.745 | CF808985 | Signal Transduction |
| 8 | Gma.5689.3.S1_s_at | 8.693 | BE657645 | Protein Destination & Storage |
| 9 | GmaAffx.50677.1.S1_s_at | 6.936 | BI971744 | No Homology to Known Proteins |
| 10 | Gma.152.1.S1_at | 6.928 | Y10492.1 | Metabolism |
| 11 | Gma.8416.1.S1_at | 6.715 | U13987.1 | Metabolism |
| 11 | Gma.8416.1.S1_at | 6.715 | U13987.1 | Metabolism |
| 11 | Gma.8416.1.S1_at | 6.715 | U13987.1 | Metabolism |
| 12 | GmaAffx.9866.1.S1_at | 6.693 | BM524432 | Metabolism |
| 13 | Gma.17789.1.S1_at | 6.487 | BG511704 | Unclassified - Hypothetical |
| | | | | Protein Supported by cDNA |
| 14 | Gma.5629.1.S1_at | 6.247 | BQ740550 | Disease & Defense |
| 15 | Gma.320.1.S1_at | 5.971 | AF019116.1 | Disease & Defense |
| 16 | Gma.3804.1.A1_at | 5.568 | AW310249 | Disease & Defense |
| 17 | GmaAffx.11276.1.S1_s_at | 5.327 | AW567677 | Unclassified - Hypothetical |
| | | | | Protein Supported by cDNA |
| 18 | GmaAffx.73185.1.S1_at | 5.042 | BI701903 | No Homology to Known Proteins |
| 19 | GmaAffx.90960.1.A1_s_at | 4.941 | CF806685 | Signal Transduction |
| 20 | GmaAffx.87679.1.S1_at | 4.865 | BM523734 | Metabolism |
| 21 | Gma.15523.1.S1_s_at | 4.791 | BE475550 | Cell Structure |
| 22 | Gma.17961.1.S1_at | 4.65 | AW350363 | Protein Destination & Storage |
| 23 | GmaAffx.10340.1.S1_at | 4.508 | CA938421 | No Homology to Known Proteins |
| 24 | GmaAffx.93073.1.S1_s_at | 4.465 | CF808798 | Cell Structure |
| 25 | Gma.16832.1.S1_s_at | 4.411 | L22029.1 | No Homology to Known Proteins |
| 26 | Gma.5816.1.S1_at | 4.336 | BI787790 | Unclassified - Hypothetical |
| | | | | Protein Supported by cDNA |
| 27 | Gma.3888.2.S1_at | 4.335 | BM139532 | Unclassified - Hypothetical |
| | | | | Protein NOT Supported by cDNA |
| 28 | GmaAffx.87679.2.S1_s_at | 4.332 | AW102083 | Metabolism |
| 29 | GmaAffx.18566.1.A1_at | 4.291 | BU550417 | Unclassified - Hypothetical |
| | | | | Protein NOT Supported by cDNA |
| 30 | GmaAffx.93541.2.S1_at | 4.208 | CF805889 | Protein Destination & Storage |
| 31 | Gma.1221.1.S1_s_at | 4.204 | L23854.1 | Metabolism |
| 31 | Gma.1221.1.S1_s_at | 4.204 | L23854.1 | Metabolism |
| 31 | Gma.1221.1.S1_s_at | 4.204 | L23854.1 | Metabolism |
| 32 | Gma.16425.3.S1_s_at | 4.126 | AW508123 | Unclassified - Protein with |
| | | | | Unknown Function |
| 33 | Gma.16425.2.S1_s_at | 4.078 | AW099501 | Unclassified - Hypothetical |
| | | | | Protein NOT Supported by cDNA |
| 34 | Gma.16308.1.S1_s_at | 4.027 | U64916.1 | Unclassified - Hypothetical |
| | | | | Protein NOT Supported by cDNA |
| 35 | GmaAffx.10340.1.S1_x_at | 3.77 | CA938421 | No Homology to Known Proteins |
| 36 | GmaAffx.83638.1.S1_at | 3.768 | BU549896 | Unclassified - Hypothetical |
| | | | | Protein NOT Supported by cDNA |
| 37 | GmaAffx.70875.1.S1_s_at | 3.656 | AW100693 | No Homology to Known Proteins |
| 38 | Gma.6555.1.S1_at | 3.568 | BM954521 | Unclassified - Hypothetical |
| | | | | Protein Supported by cDNA |
| 39 | Gma.4701.2.A1_a_at | 3.545 | BM526048 | Energy |
| 40 | Gma.1400.2.S1_at | 3.516 | BG510739 | Signal Transduction |
| 41 | GmaAffx.67477.2.S1_at | 3.453 | AW509184 | Cell Structure |
| 42 | GmaAffx.91346.1.S1_x_at | 3.41 | CF807071 | Unclassified - Hypothetical |
| | | | | Protein Supported by cDNA |
| 43 | GmaAffx.11858.1.A1_s_at | 3.402 | AW351023 | Metabolism |
| 44 | GmaAffx.92537.1.S1_at | 3.401 | CF808262 | Energy |
| 45 | Gma.3639.1.S1_at | 3.4 | AB083030.1 | Protein Destination & Storage |
| 46 | Gma.6288.1.S1_at | 3.388 | AW348556 | Transporter |
| 47 | Gma.5483.1.S1_s_at | 3.362 | CD414581 | Transcription |
| 48 | GmaAffx.85409.1.S1_at | 3.337 | BF598665 | Unclassified - Hypothetical |
| | | | | Protein NOT Supported by cDNA |
| 49 | Gma.17559.1.S1_at | 3.31 | BG508388 | No Homology to Known Proteins |
| 50 | GmaAffx.87812.2.S1_at | 3.31 | BI971676 | Signal Transduction |
| 51 | GmaAffx.87526.1.S1_at | 3.286 | BM954581 | Unclassified - Hypothetical |
| | | | | Protein Supported by cDNA |
| 52 | Gma.13447.1.A1_at | 3.285 | CD398305 | No Homology to Known Proteins |
| 53 | GmaAffx.93462.1.S1_s_at | 3.272 | CF809187 | Signal Transduction |
| 54 | GmaAffx.24352.1.S1_at | 3.243 | BU544287 | No Homology to Known Proteins |
| 55 | GmaAffx.81930.1.S1_at | 3.194 | BG789998 | Unclassified - Hypothetical |
| | | | | Protein Supported by cDNA |
| 56 | Gma.8896.1.S1_at | 3.179 | BQ252812 | Transcription |
| 57 | Gma.14471.2.S1_at | 3.168 | CD408292 | Transporter |
| 58 | Gma.3149.1.S1_a_at | 3.154 | BU544575 | No Homology to Known Proteins |
| 59 | GmaAffx.68088.1.S1_at | 3.1 | BG507880 | Signal Transduction |
| 60 | GmaAffx.54265.1.S1_s_at | 3.068 | BU546096 | Signal Transduction |
| 61 | Gma.4226.2.S1_at | 3.037 | BM568170 | Unclassified - Hypothetical |
| | | | | Protein Supported by cDNA |
| 62 | Gma.8301.2.S1_at | 3.014 | BU544524 | Signal Transduction |
| 63 | GmaAffx.62542.1.S1_at | 2.983 | BE659136 | Unclassified - Hypothetical |
| | | | | Protein NOT Supported by cDNA |
| 64 | GmaAffx.31180.1.A1_at | 2.939 | BE820254 | No Homology to Known Proteins |
| 65 | GmaAffx.85264.1.S1_at | 2.932 | BE058913 | Metabolism |
| 66 | GmaAffx.2743.1.S1_s_at | 2.913 | BM568087 | Signal Transduction |
| 67 | Gma.43.1.A1_at | 2.884 | L12258.1 | Transporter |
| 68 | GmaAffx.40860.2.S1_at | 2.85 | BG509282 | Disease & Defense |
| 69 | GmaAffx.43291.1.S1_at | 2.782 | AI442332 | Metabolism |
| 70 | GmaAffx.85374.1.S1_at | 2.76 | BI317002 | Signal Transduction |
| 71 | Gma.11259.1.S1_at | 2.733 | AI442316 | Unclassified - Hypothetical |
| | | | | Protein Supported by cDNA |
| 72 | GmaAffx.4905.1.S1_at | 2.73 | BQ453808 | Metabolism |
| 73 | Gma.7474.1.A1_at | 2.672 | BU547968 | Unclassified - Hypothetical |
| | | | | Protein Supported by cDNA |
| 74 | Gma.17605.1.S1_at | 2.637 | BI968208 | Secondary Metabolism |
| 75 | Gma.6266.1.A1_at | 2.593 | CD413418 | Unclassified - Protein with |
| | | | | Unknown Function |
| 76 | Gma.340.1.S1_at | 2.59 | AW350127 | Cell Structure |
| 77 | GmaAffx.16502.1.A1_at | 2.585 | BE659118 | No Homology to Known Proteins |
| 78 | GmaAffx.81783.1.S1_at | 2.559 | AW101961 | Signal Transduction |
| 79 | GmaAffx.8098.1.S1_at | 2.546 | AW156410 | No Homology to Known Proteins |
| 80 | Gma.184.1.S1_at | 2.534 | AW101261 | Unclassified - Hypothetical |
| | | | | Protein Supported by cDNA |
| 81 | GmaAffx.18518.1.S1_at | 2.498 | BI700329 | Cell Structure |
| 82 | GmaAffx.87812.1.S1_at | 2.455 | AW831390 | Signal Transduction |
| 83 | GmaAffx.87547.2.S1_at | 2.45 | BQ080473 | Cell Structure |
| 84 | GmaAffx.91346.1.S1_at | 2.437 | CF807071 | Unclassified - Hypothetical |
| | | | | Protein Supported by cDNA |
| 85 | GmaAffx.65829.1.A1_at | 2.428 | CD392418 | Transcription |
| 86 | GmaAffx.78636.1.S1_at | 2.428 | BQ627652 | No Homology to Known Proteins |
| 87 | GmaAffx.5264.1.S1_s_at | 2.415 | BM521512 | Disease & Defense |
| 88 | GmaAffx.89512.1.A1_at | 2.403 | CK606530 | No Homology to Known Proteins |
| 89 | Gma.368.1.S1_at | 2.387 | AW349594 | No Homology to Known Proteins |
| 90 | Gma.4396.1.S1_at | 2.368 | BQ081294 | No Homology to Known Proteins |
| 91 | Gma.1521.1.S1_at | 2.363 | CD394504 | Cell Structure |
| 92 | GmaAffx.21211.1.S1_at | 2.335 | BU550606 | Protein Destination & Storage |
| 93 | Gma.2488.1.S1_at | 2.327 | CD406079 | Protein Destination & Storage |
| 94 | GmaAffx.33259.1.S1_at | 2.272 | BQ785322 | Unclassified - Hypothetical |
| | | | | Protein Supported by cDNA |
| 95 | Gma.666.1.S1_at | 2.239 | AW310734 | Unclassified - Protein with |
| | | | | Unknown Function |
| 96 | Gma.3555.1.S1_at | 2.23 | BU764259 | Unclassified - Hypothetical |
| | | | | Protein NOT Supported by cDNA |
| 97 | GmaAffx.56917.1.S1_at | 2.227 | AI441130 | Signal Transduction |
| 98 | GmaAffx.86059.1.S1_at | 2.206 | BF598759 | No Homology to Known Proteins |
| 99 | GmaAffx.47891.1.S1_s_at | 2.197 | BQ785880 | Cell Structure |
| 100 | GmaAffx.13625.1.A1_at | 2.195 | BU547925 | No Homology to Known Proteins |
| 101 | Gma.3777.1.S1_a_at | 2.191 | CD399954 | Unclassified - Hypothetical |
| | | | | Protein Supported by cDNA |
| 102 | GmaAffx.84266.1.S1_at | 2.19 | AI495798 | Signal Transduction |
| 103 | Gma.3777.1.S1_x_at | 2.178 | CD399954 | Unclassified - Hypothetical |
| | | | | Protein Supported by cDNA |
| 104 | Gma.5950.1.S1_s_at | 2.154 | BQ610563 | Disease & Defense |
| 105 | Gma.10334.1.S1_at | 2.104 | AW234479 | Unclassified - Hypothetical |
| | | | | Protein NOT Supported by cDNA |
| 106 | GmaAffx.10491.1.S1_at | 2.082 | AW598141 | No Homology to Known Proteins |
| 107 | Gma.6650.1.S1_at | 2.075 | BQ629283 | Unclassified - Protein with |
| | | | | Unknown Function |
| 108 | GmaAffx.90042.1.S1_at | 2.073 | CF805767 | Signal Transduction |
| 109 | GmaAffx.9618.1.S1_at | 2.067 | BM092432 | No Homology to Known Proteins |
| 110 | Gma.17816.1.A1_at | 2.059 | BI970976 | Disease & Defense |
| 111 | Gma.11257.1.S1_at | 2.016 | AW310476 | Cell Structure |
| 112 | Gma.3189.2.S1_at | 2.005 | BG511492 | Signal Transduction |
| 113 | GmaAffx.84566.3.S1_s_at | 0.499 | BG045916 | Transcription |
| 114 | GmaAffx.15862.1.S1_at | 0.497 | BM092634 | Cell Structure |
| 115 | GmaAffx.93220.1.S1_at | 0.496 | CF808945 | No Homology to Known Proteins |
| 116 | GmaAffx.15983.2.S1_s_at | 0.495 | AW279540 | Signal Transduction |
| 117 | GmaAffx.35729.1.S1_s_at | 0.495 | BE806047 | Unclassified - Hypothetical |
| | | | | Protein Supported by cDNA |
| 118 | GmaAffx.90069.1.S1_s_at | 0.494 | CF805794 | Unclassified - Hypothetical |
| | | | | Protein Supported by cDNA |
| 119 | GmaAffx.83226.1.S1_at | 0.494 | CA852250 | Transcription |
| 120 | Gma.2252.1.S1_at | 0.492 | CD406598 | Unclassified - Hypothetical |
| | | | | Protein Supported by cDNA |
| 121 | Gma.9259.1.S1_at | 0.492 | CA782161 | Transporter |
| 122 | Gma.6895.1.S1_a_at | 0.491 | BU545788 | Metabolism |
| 123 | Gma.6375.1.S1_at | 0.489 | BQ610190 | Unclassified - Hypothetical |
| | | | | Protein NOT Supported by cDNA |
| 124 | Gma.5701.3.S1_at | 0.488 | CA852693 | No Homology to Known Proteins |
| 125 | GmaAffx.81999.1.S1_at | 0.487 | AI416637 | Transcription |
| 126 | GmaAffx.55657.1.S1_at | 0.486 | BM885600 | No Homology to Known Proteins |
| 127 | Gma.12987.1.S1_at | 0.485 | CD392747 | Transcription |
| 128 | GmaAffx.60479.1.S1_at | 0.484 | BG726023 | Transcription |
| 129 | Gma.1573.2.S1_a_at | 0.483 | BI974098 | Metabolism |
| 130 | Gma.221.1.S1_at | 0.481 | U41475.1 | Metabolism |
| 131 | GmaAffx.53845.1.S1_at | 0.48 | BG406552 | Transporter |
| 132 | Gma.5294.1.S1_at | 0.479 | BE658252 | Unclassified - Hypothetical |
| | | | | Protein Supported by cDNA |
| 133 | GmaAffx.73857.1.S1_at | 0.479 | BM885538 | Unclassified - Hypothetical |
| | | | | Protein Supported by cDNA |
| 134 | Gma.17798.2.S1_at | 0.479 | CD412748 | No Homology to Known Proteins |
| 135 | Gma.13273.1.A1_at | 0.478 | CD397107 | Unclassified - Hypothetical |
| | | | | Protein Supported by cDNA |
| 136 | GmaAffx.14141.1.A1_at | 0.476 | BI973009 | No Homology to Known Proteins |
| 137 | Gma.6129.2.S1_at | 0.475 | AW186006 | Protein Synthesis |
| 138 | Gma.2565.1.S1_at | 0.475 | BG652533 | Unclassified - Hypothetical |
| | | | | Protein Supported by cDNA |
| 139 | Gma.5331.1.S1_a_at | 0.475 | BI892702 | Transcription |
| 140 | Gma.13035.1.S1_at | 0.474 | CD396536 | No Homology to Known Proteins |
| 141 | GmaAffx.15471.1.S1_at | 0.47 | BQ611747 | Transcription |
| 142 | GmaAffx.74256.1.S1_at | 0.469 | BE057293 | No Homology to Known Proteins |
| 143 | GmaAffx.69198.1.S1_at | 0.468 | BM526951 | Unclassified - Hypothetical |
| | | | | Protein Supported by cDNA |
| 144 | GmaAffx.33527.1.S1_at | 0.468 | BU761195 | No Homology to Known Proteins |
| 145 | Gma.7040.1.A1_at | 0.467 | BE346424 | Transcription |
| 146 | GmaAffx.494.1.S1_at | 0.464 | AI938330 | Cell Structure |
| 147 | GmaAffx.93116.1.S1_at | 0.463 | CF806290 | Disease & Defense |
| 148 | GmaAffx.47288.1.S1_at | 0.461 | BU764979 | Unclassified - Hypothetical |
| | | | | Protein Supported by cDNA |
| 149 | Gma.3578.1.S1_s_at | 0.458 | AW156522 | Disease & Defense |
| 150 | GmaAffx.23292.1.A1_at | 0.457 | BI967795 | No Homology to Known Proteins |
| 151 | Gma.1619.1.S1_at | 0.457 | AW472195 | Unclassified - Hypothetical |
| | | | | Protein Supported by cDNA |
| 152 | Gma.17070.1.S1_at | 0.456 | BE822463 | Disease & Defense |
| 153 | Gma.11026.2.S1_s_at | 0.455 | BQ080041 | Disease & Defense |
| 154 | GmaAffx.90899.1.S1_at | 0.454 | CF806624 | Unclassified - Hypothetical |
| | | | | Protein NOT Supported by cDNA |
| 155 | GmaAffx.84964.1.S1_at | 0.453 | AW598267 | No Homology to Known Proteins |
| 156 | GmaAffx.76699.1.S1_at | 0.451 | AW760679 | Transcription |
| 157 | GmaAffx.45003.1.S1_at | 0.445 | BI968048 | Unclassified - Hypothetical |
| | | | | Protein Supported by cDNA |
| 158 | Gma.6033.2.S1_at | 0.444 | AI416728 | Unclassified - Hypothetical |
| | | | | Protein Supported by cDNA |
| 159 | Gma.11205.1.S1_a_at | 0.442 | BF066553 | Metabolism |
| 160 | GmaAffx.34798.1.S1_at | 0.442 | BM143105 | Unclassified - Hypothetical |
| | | | | Protein Supported by cDNA |
| 161 | GmaAffx.69369.1.S1_at | 0.442 | BG511072 | Unclassified - Hypothetical |
| | | | | Protein Supported by cDNA |
| 162 | Gma.6192.1.S1_s_at | 0.44 | AW396773 | Metabolism |
| 163 | GmaAffx.21041.1.A1_at | 0.438 | AW350819 | No Homology to Known Proteins |
| 164 | Gma.13030.1.S1_a_at | 0.437 | AI748012 | Protein Destination & Storage |
| 165 | GmaAffx.90600.1.S1_at | 0.437 | CF806325 | Metabolism |
| 166 | GmaAffx.59808.1.S1_at | 0.435 | BM886228 | Transporter |
| 167 | GmaAffx.15983.2.S1_at | 0.434 | AW279540 | Signal Transduction |
| 168 | GmaAffx.11049.1.S1_at | 0.432 | BU545140 | Unclassified - Protein with |
| | | | | Unknown Function |
| 169 | GmaAffx.87417.1.S1_at | 0.43 | BM568072 | Metabolism |
| 170 | GmaAffx.48042.1.S1_at | 0.429 | AI960210 | Transporter |
| 171 | GmaAffx.12014.1.A1_at | 0.428 | CA802431 | Unclassified - Hypothetical |
| | | | | Protein Supported by cDNA |
| 172 | GmaAffx.59210.1.S1_at | 0.427 | BI967182 | Secondary Metabolism |
| 173 | Gma.3450.1.S1_at | 0.425 | BE657895 | Unclassified - Hypothetical |
| | | | | Protein Supported by cDNA |
| 174 | GmaAffx.15471.3.S1_x_at | 0.425 | BE346777 | Transcription |
| 175 | GmaAffx.91428.1.A1_at | 0.423 | CF807137 | Metabolism |
| 176 | Gma.1061.1.A1_at | 0.421 | CD408183 | Metabolism |
| 177 | Gma.10536.1.S1_at | 0.42 | BG508300 | No Homology to Known Proteins |
| 178 | Gma.1495.1.S1_at | 0.418 | BI973545 | No Homology to Known Proteins |
| 179 | GmaAffx.53099.1.S1_at | 0.418 | BG043340 | Unclassified - Hypothetical |
| | | | | Protein NOT Supported by cDNA |
| 180 | Gma.5586.2.S1_at | 0.416 | CD398384 | Transcription |
| 181 | Gma.3384.1.S1_at | 0.414 | BE659400 | Unclassified - Protein with |
| | | | | Unknown Function |
| 182 | GmaAffx.948.1.S1_at | 0.414 | BG155389 | Protein Destination & Storage |
| 183 | Gma.6684.1.S1_at | 0.413 | AW459859 | Signal Transduction |
| 184 | Gma.4687.1.S1_at | 0.412 | AW309716 | Secondary Metabolism |
| 185 | Gma.6192.1.S1_at | 0.411 | AW396773 | Metabolism |
| 186 | GmaAffx.74160.1.S1_at | 0.409 | BQ740845 | Protein Destination & Storage |
| 187 | Gma.16910.1.S1_at | 0.406 | BU549219 | Unclassified - Protein with |
| | | | | Unknown Function |
| 188 | Gma.1394.1.S1_at | 0.4 | BG839300 | Metabolism |
| 189 | GmaAffx.42732.1.S1_at | 0.399 | BU765857 | Protein Destination & Storage |
| 190 | GmaAffx.93342.1.S1_s_at | 0.399 | CF809067 | Disease & Defense |
| 191 | GmaAffx.70005.1.S1_s_at | 0.395 | CA936335 | Secondary Metabolism |
| 192 | Gma.1951.2.S1_a_at | 0.392 | CA783485 | Protein Destination & Storage |
| 193 | GmaAffx.77203.1.S1_at | 0.39 | BI970687 | Unclassified - Hypothetical |
| | | | | Protein Supported by cDNA |
| 194 | Gma.1394.2.S1_at | 0.389 | BI470370 | Metabolism |
| 195 | Gma.4252.1.S1_at | 0.389 | BE821208 | Metabolism |
| 196 | Gma.3578.1.S1_at | 0.386 | AW156522 | Disease & Defense |
| 197 | Gma.3420.1.S1_at | 0.385 | CD406082 | Unclassified - Hypothetical |
| | | | | Protein Supported by cDNA |
| 198 | GmaAffx.15196.1.S1_at | 0.384 | BU549436 | Metabolism |
| 199 | Gma.16359.1.A1_at | 0.384 | BI970359 | Metabolism |
| 200 | Gma.6033.1.S1_at | 0.382 | BI967767 | Unclassified - Hypothetical |
| | | | | Protein Supported by cDNA |
| 201 | GmaAffx.57432.2.S1_at | 0.38 | CA819434 | No Homology to Known Proteins |
| 202 | GmaAffx.90600.1.S1_s_at | 0.379 | CF806325 | Metabolism |
| 203 | GmaAffx.93212.1.S1_s_at | 0.379 | CF807524 | Transporter |
| 204 | Gma.2540.2.S1_s_at | 0.378 | BI787366 | No Homology to Known Proteins |
| 205 | Gma.4099.1.S1_at | 0.378 | BG651846 | Metabolism |
| 206 | Gma.17402.1.A1_at | 0.376 | BE611084 | No Homology to Known Proteins |
| 207 | Gma.13418.3.S1_at | 0.372 | CA938391 | Transporter |
| 208 | Gma.1326.1.S1_at | 0.37 | CD397515 | Secondary Metabolism |
| 209 | Gma.7227.1.S1_at | 0.37 | BU551112 | Protein Destination & Storage |
| 210 | GmaAffx.78358.2.S1_at | 0.37 | BU762686 | Metabolism |
| 211 | Gma.1490.1.S1_at | 0.369 | AB000097.1 | Cell Structure |
| 212 | GmaAffx.58125.1.A1_at | 0.368 | BE347561 | Transcription |
| 213 | GmaAffx.36470.1.A1_at | 0.367 | BU550552 | Unclassified - Hypothetical |
| | | | | Protein Supported by cDNA |
| 214 | Gma.7831.1.S1_s_at | 0.367 | CD393602 | Transporter |
| 215 | GmaAffx.87900.1.S1_at | 0.367 | BI892943 | Protein Destination & Storage |
| 216 | Gma.5513.1.S1_at | 0.367 | AW310453 | Metabolism |
| 217 | GmaAffx.91731.1.S1_s_at | 0.363 | CF807456 | Secondary Metabolism |
| 218 | Gma.2066.1.S1_a_at | 0.362 | BG363704 | No Homology to Known Proteins |
| 219 | Gma.11166.1.S1_x_at | 0.359 | AW705829 | Metabolism |
| 220 | GmaAffx.71308.1.S1_at | 0.358 | BF595112 | Transcription |
| 221 | GmaAffx.82936.1.S1_at | 0.358 | AI938364 | Unclassified - Protein with |
| | | | | Unknown Function |
| 222 | GmaAffx.93328.1.S1_s_at | 0.357 | CF809053 | Secondary Metabolism |
| 223 | GmaAffx.93591.1.S1_s_at | 0.354 | CF809316 | Metabolism |
| 224 | GmaAffx.64807.1.S1_at | 0.354 | AW201972 | No Homology to Known Proteins |
| 225 | Gma.857.1.S1_at | 0.352 | BE822264 | No Homology to Known Proteins |
| 226 | Gma.7580.1.S1_at | 0.351 | BE057763 | Unclassified - Hypothetical |
| | | | | Protein Supported by cDNA |
| 227 | Gma.3591.1.S1_a_at | 0.35 | BE440266 | Secondary Metabolism |
| 228 | Gma.5162.1.A1_at | 0.347 | BI971156 | Signal Transduction |
| 229 | GmaAffx.17892.2.S1_at | 0.345 | BQ628677 | No Homology to Known Proteins |
| 230 | Gma.11166.1.S1_s_at | 0.344 | AW705829 | Metabolism |
| 231 | Gma.9647.1.S1_at | 0.343 | BE820997 | Cell Structure |
| 232 | GmaAffx.11242.1.S1_s_at | 0.343 | AW317461 | Protein Destination & Storage |
| 233 | GmaAffx.80584.1.S1_at | 0.343 | BU083387 | Transcription |
| 234 | Gma.5760.2.S1_at | 0.341 | BQ298381 | Disease & Defense |
| 235 | Gma.5929.1.S1_at | 0.341 | BQ610446 | No Homology to Known Proteins |
| 236 | Gma.13150.1.A1_at | 0.341 | CD411577 | Unclassified - Protein with |
| | | | | Unknown Function |
| 237 | GmaAffx.53324.1.A1_at | 0.341 | AW309251 | Transcription |
| 238 | Gma.4643.1.S1_at | 0.339 | BU547906 | Transcription |
| 239 | Gma.398.2.S1_at | 0.338 | BU925900 | Metabolism |
| 240 | Gma.17516.1.S1_at | 0.336 | CD396417 | Transporter |
| 241 | GmaAffx.68507.1.S1_at | 0.333 | BI699405 | Transcription |
| 242 | Gma.16500.1.S1_at | 0.332 | BE059054 | Metabolism |
| 243 | GmaAffx.32011.1.S1_at | 0.332 | BI701198 | No Homology to Known Proteins |
| 244 | GmaAffx.83715.1.S1_at | 0.331 | BI785710 | Transporter |
| 245 | Gma.4728.1.A1_at | 0.328 | BI968944 | Secondary Metabolism |
| 246 | GmaAffx.79516.1.A1_at | 0.326 | CD400596 | Transcription |
| 247 | GmaAffx.64967.1.S1_at | 0.325 | BU546115 | Secondary Metabolism |
| 248 | Gma.1951.1.S1_at | 0.321 | BQ079657 | Protein Destination & Storage |
| 249 | GmaAffx.63464.1.S1_at | 0.318 | BG156739 | No Homology to Known Proteins |
| 250 | Gma.10591.2.S1_at | 0.318 | CD414199 | Metabolism |
| 251 | Gma.1314.1.S1_at | 0.314 | U63726.1 | Metabolism |
| 252 | GmaAffx.88546.1.S1_at | 0.313 | BE801134 | Metabolism |
| 253 | GmaAffx.64794.1.S1_at | 0.313 | CA801749 | Unclassified - Hypothetical |
| | | | | Protein Supported by cDNA |
| 254 | Gma.1394.2.S1_a_at | 0.313 | BI470370 | Metabolism |
| 255 | Gma.12227.1.S1_at | 0.312 | BG511671 | Unclassified - Hypothetical |
| | | | | Protein NOT Supported by cDNA |
| 256 | GmaAffx.32011.2.S1_at | 0.312 | BM084966 | Transcription |
| 257 | Gma.177.1.S1_at | 0.312 | AF004806.1 | Protein Destination & Storage |
| 258 | Gma.8022.1.S1_at | 0.311 | BQ628998 | Disease & Defense |
| 259 | GmaAffx.27331.1.S1_at | 0.31 | BM520297 | Unclassified - Hypothetical |
| | | | | Protein NOT Supported by cDNA |
| 260 | GmaAffx.33677.1.S1_at | 0.308 | CA782582 | No Homology to Known Proteins |
| 261 | GmaAffx.66215.1.S1_at | 0.308 | AI443109 | Signal Transduction |
| 262 | GmaAffx.77832.1.S1_at | 0.305 | BI971626 | Unclassified - Hypothetical |
| | | | | Protein NOT Supported by cDNA |
| 263 | Gma.728.1.A1_at | 0.3 | BE658779 | No Homology to Known Proteins |
| 264 | Gma.4874.1.A1_at | 0.298 | AI938293 | No Homology to Known Proteins |
| 265 | Gma.11028.2.S1_s_at | 0.297 | CA782711 | Metabolism |
| 266 | GmaAffx.50980.1.S1_s_at | 0.296 | BE823095 | Signal Transduction |
| 267 | Gma.5513.1.S1_x_at | 0.295 | AW310453 | Metabolism |
| 268 | GmaAffx.78163.1.S1_s_at | 0.293 | BM520857 | Signal Transduction |
| 269 | GmaAffx.11172.1.A1_at | 0.293 | CD404556 | No Homology to Known Proteins |
| 270 | Gma.8530.1.S1_at | 0.292 | AF109720.1 | Protein Destination & Storage |
| 271 | GmaAffx.5255.1.S1_at | 0.292 | BG362929 | Transcription |
| 272 | Gma.3118.1.S1_s_at | 0.29 | BQ786048 | Disease & Defense |
| 273 | Gma.8058.1.A1_at | 0.289 | BM271479 | Signal Transduction |
| 274 | Gma.7437.1.S1_at | 0.289 | BU547705 | Unclassified - Hypothetical |
| | | | | Protein Supported by cDNA |
| 275 | Gma.3744.2.S1_a_at | 0.288 | BI892693 | Unclassified - Protein With |
| | | | | Unknown Function |
| 276 | GmaAffx.56319.1.S1_at | 0.288 | BE802287 | Metabolism |
| 277 | GmaAffx.58712.1.S1_at | 0.287 | BF325034 | Transporter |
| 278 | GmaAffx.3369.2.A1_at | 0.286 | BU550316 | Cell Structure |
| 279 | Gma.17688.1.S1_s_at | 0.285 | CA937360 | Transcription |
| 280 | Gma.5469.1.S1_at | 0.284 | AW349200 | No Homology to Known Proteins |
| 281 | GmaAffx.89624.1.A1_s_at | 0.279 | CK606100 | Transcription |
| 282 | Gma.595.1.S1_at | 0.273 | AB004271.1 | Metabolism |
| 283 | Gma.3744.1.S1_at | 0.27 | CD417438 | Unclassified - Protein With |
| | | | | Unknown Function |
| 284 | GmaAffx.70435.2.S1_at | 0.266 | AW705853 | Transcription |
| 285 | Gma.12731.2.A1_s_at | 0.263 | CD395848 | Unclassified - Hypothetical |
| | | | | Protein NOT Supported by cDNA |
| 286 | GmaAffx.34657.1.A1_s_at | 0.261 | AW351012 | No Homology to Known Proteins |
| 287 | Gma.2086.1.S1_at | 0.258 | AF035671.1 | Metabolism |
| 288 | Gma.10896.3.A1_at | 0.253 | BI316837 | Metabolism |
| 289 | Gma.968.1.S1_at | 0.253 | D28876.1 | Protein Destination & Storage |
| 290 | GmaAffx.26908.1.S1_at | 0.253 | BF425834 | Secondary Metabolism |
| 291 | GmaAffx.25612.1.S1_at | 0.252 | AW317577 | No Homology to Known Proteins |
| 292 | GmaAffx.84424.1.S1_at | 0.252 | AW311543 | Metabolism |
| 293 | GmaAffx.17918.1.S1_at | 0.25 | BG157024 | No Homology to Known Proteins |
| 294 | Gma.10904.1.S1_s_at | 0.247 | CD408184 | Protein Destination & Storage |
| 295 | GmaAffx.45957.2.S1_at | 0.247 | BI974135 | No Homology to Known Proteins |
| 296 | Gma.11731.1.A1_at | 0.243 | CD392320 | Unclassified - Protein with |
| | | | | Unknown Function |
| 297 | GmaAffx.32606.1.S1_at | 0.241 | BM270707 | Unclassified - Hypothetical |
| | | | | Protein NOT Supported by cDNA |
| 298 | GmaAffx.80188.1.A1_at | 0.239 | AW349832 | No Homology to Known Proteins |
| 299 | GmaAffx.57970.1.S1_at | 0.238 | BM886862 | Transcription |
| 300 | GmaAffx.40923.1.S1_at | 0.238 | BG653848 | Disease & Defense |
| 301 | GmaAffx.52826.3.S1_at | 0.237 | BF596503 | Signal Transduction |
| 302 | GmaAffx.10582.1.S1_at | 0.234 | BM523687 | Transcription |
| 303 | GmaAffx.82263.1.S1_s_at | 0.234 | AW568454 | No Homology to Known Proteins |
| 304 | Gma.12068.1.A1_at | 0.232 | BU547739 | Transporter |
| 305 | GmaAffx.71308.2.A1_at | 0.23 | AW569888 | No Homology to Known Proteins |
| 306 | Gma.12684.1.S1_at | 0.226 | CD394773 | Transcription |
| 307 | GmaAffx.19118.1.S1_at | 0.225 | BM523731 | Unclassified - Hypothetical |
| | | | | Protein NOT Supported by cDNA |
| 308 | Gma.1527.1.S1_x_at | 0.22 | BI968185 | Secondary Metabolism |
| 309 | Gma.5331.1.S1_at | 0.22 | BI892702 | Transcription |
| 310 | GmaAffx.30792.1.A1_at | 0.22 | BU544815 | Unclassified - Hypothetical |
| | | | | Protein Supported by cDNA |
| 311 | Gma.17688.1.S1_at | 0.219 | CA937360 | Transcription |
| 312 | Gma.7824.1.S1_at | 0.219 | BU550492 | Metabolism |
| 313 | Gma.1768.1.S1_s_at | 0.219 | BE820777 | Unclassified - Hypothetical |
| | | | | Protein Supported by cDNA |
| 314 | GmaAffx.81362.1.S1_at | 0.218 | BF324988 | Cell Structure |
| 315 | GmaAffx.38555.2.S1_at | 0.214 | BI425873 | No Homology to Known Proteins |
| 316 | Gma.11873.1.S1_at | 0.207 | CA819977 | Unclassified - Protein with |
| | | | | Unknown Function |
| 317 | Gma.2887.2.S1_x_at | 0.206 | BI701520 | Secondary Metabolism |
| 318 | Gma.17985.2.S1_at | 0.205 | BE802263 | Metabolism |
| 319 | GmaAffx.90113.1.S1_at | 0.202 | CF806004 | Protein Destination & Storage |
| 320 | GmaAffx.26868.1.S1_at | 0.201 | BF325443 | Unclassified - Hypothetical |
| | | | | Protein NOT Supported by cDNA |
| 321 | GmaAffx.91338.1.S1_at | 0.2 | CF807063 | Unclassified - Hypothetical |
| | | | | Protein Supported by cDNA |
| 322 | GmaAffx.50980.1.S1_x_at | 0.199 | BE823095 | Signal Transduction |
| 323 | Gma.13854.1.S1_at | 0.199 | CD408166 | Metabolism |
| 324 | GmaAffx.50980.1.S1_at | 0.197 | BE823095 | Signal Transduction |
| 325 | GmaAffx.31862.1.S1_at | 0.192 | BE822501 | Unclassified - Hypothetical |
| | | | | Protein Supported by cDNA |
| 326 | GmaAffx.93212.1.S1_at | 0.191 | CF807524 | Transporter |
| 327 | GmaAffx.90395.1.S1_s_at | 0.186 | CF806120 | Unclassified - Protein with |
| | | | | Unknown Function |
| 328 | Gma.15.1.S1_at | 0.185 | U66316.1 | Unclassified - Protein with |
| | | | | Unknown Function |
| 329 | GmaAffx.9911.1.S1_at | 0.184 | BU547808 | Unclassified - Hypothetical |
| | | | | Protein NOT Supported by cDNA |
| 330 | GmaAffx.81851.1.S1_at | 0.181 | AW706751 | Unclassified - Hypothetical |
| | | | | Protein Supported by cDNA |
| 331 | GmaAffx.12665.1.A1_at | 0.176 | BE822073 | Metabolism |
| 332 | GmaAffx.89772.8.S1_at | 0.17 | CK605778 | Metabolism |
| 333 | GmaAffx.91479.1.S1_s_at | 0.17 | CF807204 | No Homology to Known Proteins |
| 334 | GmaAffx.89772.8.S1_x_at | 0.168 | CK605778 | Metabolism |
| 335 | Gma.7227.2.S1_a_at | 0.163 | AW759183 | Protein Destination & Storage |
| 336 | Gma.16477.1.S1_at | 0.159 | BE824134 | No Homology to Known Proteins |
| 337 | Gma.13062.1.S1_at | 0.157 | CD394315 | Metabolism |
| 338 | Gma.12094.1.A1_at | 0.157 | CD395088 | Unclassified - Hypothetical |
| | | | | Protein Supported by cDNA |
| 339 | Gma.15093.1.S1_at | 0.153 | CD400374 | Protein Destination & Storage |
| 340 | Gma.8187.1.S1_a_at | 0.152 | BU549133 | Signal Transduction |
| 341 | Gma.4837.1.S1_at | 0.147 | BI969343 | Transporter |
| 342 | Gma.13296.3.S1_at | 0.147 | CD418313 | Transporter |
| 343 | Gma.10904.1.S1_at | 0.141 | CD408184 | Protein Destination & Storage |
| 344 | Gma.15534.1.S1_at | 0.133 | U50081.1 | Metabolism |
| 345 | GmaAffx.93212.1.S1_x_at | 0.129 | CF807524 | Transporter |
| 346 | GmaAffx.71222.1.S1_at | 0.124 | AW460050 | Metabolism |
| 347 | Gma.10830.1.S1_at | 0.123 | BE820822 | Unclassified - Hypothetical |
| | | | | Protein Supported by cDNA |
| 348 | Gma.12393.3.S1_at | 0.121 | BM085488 | Secondary Metabolism |
| 349 | GmaAffx.295.1.A1_s_at | 0.118 | BG653267 | Protein Destination & Storage |
| 350 | GmaAffx.75697.1.S1_s_at | 0.113 | BU090518 | Unclassified - Hypothetical |
| | | | | Protein Supported by cDNA |
| 351 | GmaAffx.1785.1.S1_at | 0.112 | BF325331 | Transcription |
| 352 | Gma.1527.2.S1_at | 0.104 | BE658017 | Secondary Metabolism |
| 353 | GmaAffx.24413.1.A1_at | 0.103 | BU548368 | Metabolism |
| 354 | Gma.1527.1.S1_a_at | 0.102 | BI968185 | Secondary Metabolism |
| 355 | Gma.176.1.S1_s_at | 0.101 | AF004810.1 | Protein Destination & Storage |
| 356 | GmaAffx.90812.1.S1_at | 0.1 | CF806537 | Disease & Defense |
| 357 | Gma.17985.1.S1_at | 0.099 | CD416450 | Metabolism |
| 358 | Gma.6921.1.S1_at | 0.0986 | BE823209 | Signal Transduction |
| 359 | Gma.3774.2.S1_at | 0.0892 | CD393365 | Energy |
| 360 | GmaAffx.73698.1.S1_at | 0.0875 | BU964780 | Metabolism |
| 361 | Gma.2986.1.S1_at | 0.0773 | BM271324 | Metabolism |
| 362 | GmaAffx.12933.1.S1_at | 0.0755 | AW395787 | Transcription |
| 363 | Gma.3000.1.S1_at | 0.0728 | BE657172 | No Homology to Known Proteins |
| 364 | Gma.17326.1.A1_at | 0.0703 | AW568254 | Unclassified - Hypothetical |
| | | | | Protein NOT Supported by cDNA |
| 365 | Gma.16566.1.S1_at | 0.0682 | CD400974 | Metabolism |
| 366 | Gma.3000.2.S1_a_at | 0.0673 | BM270844 | No Homology to Known Proteins |
| 367 | Gma.9947.1.S1_at | 0.0631 | BF324269 | Unclassified - Hypothetical |
| | | | | Protein Supported by cDNA |
| 368 | GmaAffx.89407.1.A1_s_at | 0.0598 | CK606273 | Transporter |
| 369 | Gma.8318.1.A1_at | 0.0496 | BU550469 | No Homology to Known Proteins |
| 370 | Gma.3774.1.S1_at | 0.0478 | BU550698 | Energy |
| 371 | GmaAffx.10204.1.S1_at | 0.0455 | BQ298213 | Unclassified - Hypothetical |
| | | | | Protein Supported by cDNA |
| 372 | Gma.3774.2.S1_a_at | 0.0366 | CD393365 | Energy |
| 373 | GmaAffx.68621.1.A1_at | 0.0276 | BI893889 | Unclassified - Protein with |
| | | | | Unknown Function |
| 374 | Gma.2912.1.S1_at | 0.0224 | AF117884.1 | Protein Destination & Storage |
| 375 | Gma.2912.1.S1_s_at | 0.0214 | AF117884.1 | Protein Destination & Storage |

| ID | Sub-Family | Gene Family | Additional Description |
|---|---|---|---|
| 1 | | | Predicted transporter (major |
| | | | facilitator superfamily) |
| 2 | Protein Kinase | | |
| 3 | | | |
| 4 | | | |
| 5 | | | |
| 6 | | | |
| 7 | Protein Kinase | | |
| 8 | | | Gelatinase A and related |
| | | | matrix metalloproteases |
| 9 | | | |
| 10 | Cytochrome P450 | CYP93D | Cytochrome P450 CYP2 |
| | | | subfamily |
| 11 | | | Cytochrome b5 |
| 11 | | | NADH-cytochrome b-5 |
| | | | reductase |
| 11 | | | Sulfite oxidase, |
| | | | molybdopterin-binding |
| | | | component |
| 12 | | | |
| 13 | | | |
| 14 | | | |
| 15 | | | |
| 16 | | | |
| 17 | | | |
| 18 | | | |
| 19 | Protein Kinase | | |
| 20 | Trehalose | | Trehalose-6-phosphate |
| | Biosynthesis | | synthase component TPS1 |
| | | | and related subunits |
| 21 | | | |
| 22 | Storage Protein | | |
| 23 | | | |
| 24 | | | |
| 25 | | | |
| 26 | | | Aldehyde reductase |
| 27 | | | Aldehyde reductase |
| 28 | Trehalose | | Trehalose-6-phosphate |
| | Biosynthesis | | synthase component TPS1 |
| | | | and related subunits |
| 29 | | | |
| 30 | | | Gelatinase A and related |
| | | | matrix metalloproteases |
| 31 | | | Cytochrome b5 |
| 31 | | | NADH-cytochrome b-5 |
| | | | reductase |
| 31 | | | Sulfite oxidase, |
| | | | molybdopterin-binding |
| | | | component |
| 32 | | | Copper chaperone |
| 33 | | | Copper chaperone |
| 34 | | | Copper chaperone |
| 35 | | | |
| 36 | | | |
| 37 | | | |
| 38 | | | |
| 39 | | | Thioredoxin |
| 40 | Protein Kinase | AtC1PKs | Serine/threonine protein |
| | | | kinase |
| 41 | | | |
| 42 | | | |
| 43 | Phospholipid | | Glycosylphosphatidylinositol- |
| | Biosynthesis | | specific phospholipase C |
| 44 | | | Thioredoxin |
| 45 | Storage Protein | | |
| 46 | | | Aquaporin (major intrinsic |
| | | | protein family) |
| 47 | Transcription Factor | Basic Leucine | |
| | | Zipper (bZIP) | |
| 48 | | | Copper chaperone |
| 49 | | | |
| 50 | Protein Kinase | | |
| 51 | | | |
| 52 | | | |
| 53 | Protein Kinase | AtC1PKs | Serine/threonine protein |
| | | | kinase |
| 54 | | | |
| 55 | | | |
| 56 | Transcription Factor | AP2/EREBP | |
| 57 | | | Aquaporin (major intrinsic |
| | | | protein family) |
| 58 | | | |
| 59 | Protein Kinase | | |
| 60 | Protein Kinase | | |
| 61 | | | |
| 62 | Calcium Binding | | |
| 63 | | | |
| 64 | | | |
| 65 | | | |
| 66 | Protein Kinase | | |
| 67 | | | Aquaporin (major intrinsic |
| | | | protein family) |
| 68 | | | |
| 69 | | | Lipid phosphate phosphatase |
| | | | and related enzymes of the |
| | | | PAP2 family |
| 70 | Protein Kinase | | |
| 71 | | | |
| 72 | | | Predicted lipase |
| 73 | | | |
| 74 | Flavonoid | | |
| | Biosynthesis | | |
| 75 | | | |
| 76 | | | |
| 77 | | | |
| 78 | Protein Kinase | | Serine/threonine protein |
| | | | kinase |
| 79 | | | |
| 80 | | | |
| 81 | | | |
| 82 | Protein Kinase | | |
| 83 | | | Beta-glucosidase, lactase |
| | | | phlorizinhydrolase, and |
| | | | related proteins |
| 84 | | | |
| 85 | Transcription Factor | AP2/EREBP | |
| 86 | | | |
| 87 | | | |
| 88 | | | |
| 89 | | | |
| 90 | | | |
| 91 | | | Beta tubulin |
| 92 | | | Molecular chaperone (HSP90 |
| | | | family) |
| 93 | | | Serine carboxypeptidases |
| | | | (lysosomal cathepsin A) |
| 94 | | | |
| 95 | Hormone Induced | | Voltage-gated shaker-like K+ |
| | | | channel, subunit |
| | | | beta/KCNAB |
| 96 | | | |
| 97 | Protein Kinase | | Serine/threonine protein |
| | | | kinase |
| 98 | | | |
| 99 | | | |
| 100 | | | |
| 101 | | | |
| 102 | Protein Kinase | AtCIPKs | Serine/threonine protein |
| | | | kinase |
| 103 | | | |
| 104 | | | |
| 105 | | | |
| 106 | | | |
| 107 | | | |
| 108 | Calcium Binding | | Calmodulin and related |
| | | | proteins (EF-Hand |
| | | | superfamily) |
| 109 | | | |
| 110 | | | |
| 111 | | | |
| 112 | MAPKK | | Mitogen-activated protein |
| | | | kinase kinase (MAP2K) |
| 113 | Transcription Factor | MYB | Transcription factor, Myb |
| | | | superfamily |
| 114 | | | |
| 115 | | | |
| 116 | Protein Phosphatase | | Serine/threonine protein |
| | | | phosphatase |
| 117 | | | Predicted E3 ubiquitin ligase |
| 118 | | | Leucine rich repeat proteins, |
| | | | some proteins contain F-box |
| 119 | Transcription Factor | Unclassified | |
| 120 | | | |
| 121 | | | |
| 122 | | | Alanine-glyoxylate |
| | | | aminotransferase AGT2 |
| 123 | | | AAA+-type ATPase |
| 124 | | | |
| 125 | Transcription Factor | Homeodomain | Transcription factor MEIS1 |
| | | | and related HOX domain |
| | | | proteins |
| 126 | | | |
| 127 | Transcription Factor | Basic Helix- | |
| | | Loop-Helix | |
| | | (bHLH) | |
| 128 | Transcription Factor | Homeodomain | Transcription factor MEISI |
| | | | and related HOX domain |
| | | | proteins |
| 129 | | | |
| 130 | | | Phosphoinositide-specific |
| | | | phospholipase C |
| 131 | | | Sulfate/bicarbonate/oxalate |
| | | | exchanger SAT-I and related |
| | | | transporters (SLC26 family) |
| 132 | | | |
| 133 | | | |
| 134 | | | |
| 135 | | | |
| 136 | | | |
| 137 | | | Translation initiation factor |
| | | | 5A (eIF-5A) |
| 138 | | | |
| 139 | Transcription Factor | NAC Domain | |
| 140 | | | |
| 141 | Transcription Factor | MYB | Transcription factor, Myb |
| | | | superfamily |
| 142 | | | |
| 143 | | | Leucine rich repeat proteins. |
| | | | some proteins contain F-box |
| 144 | | | |
| 145 | Transcription Factor | AP2/EREBP | |
| 146 | | | Chitinase |
| 147 | | | |
| 148 | | | GTPase Rab18, small G |
| | | | protein superfamily |
| 149 | | | Acyl-CoA synthetase |
| 150 | | | |
| 151 | | | |
| 152 | | | O-methyltransferase |
| 153 | | | |
| 154 | | | |
| 155 | | | |
| 156 | Transcription Factor | Basic Helix- | |
| | | Loop-Helix | |
| | | (bHLH) | |
| 157 | | | Uncharacterized conserved |
| | | | protein |
| 158 | | | |
| 159 | | | Transketolase |
| 160 | | | |
| 161 | | | Galactosyltransferases |
| 162 | | | |
| 163 | | | |
| 164 | | | |
| 165 | | | |
| 166 | | | Predicted transporter (major |
| | | | facilitator superfamily) |
| 167 | Protein Phosphatase | | Serine/threonine protein |
| | | | phosphatase |
| 168 | | | Phosphatidylethanolamine |
| | | | binding protein |
| 169 | | | |
| 170 | | | Aquaporin (major intrinsic |
| | | | protein family) |
| 171 | | | NADH pyrophosphatase I of |
| | | | the Nudix family of |
| | | | hydrolases |
| 172 | Ethylene | | Iron/ascorbate family |
| | Biosynthesis | | oxidoreductases |
| 173 | | | |
| 174 | Transcription Factor | MYB | Transcription factor, Myb |
| | | | superfamily |
| 175 | | | |
| 176 | | | |
| 177 | | | |
| 178 | | | |
| 179 | | | Alpha-L-fucosidase |
| 180 | Transcription Factor | Unclassified | CCAAT-binding factor, |
| | | | subunit B (HAP2) |
| 181 | | | Acyl-CoA reductase |
| 182 | | | Ubiquitin fusion-degradation |
| | | | protein |
| 183 | Unclassified | | Lanthionine synthetase C-like |
| | | | protein 1 |
| 184 | | | |
| 185 | | | |
| 186 | | | |
| 187 | | | |
| 188 | | | |
| 189 | Ubiquitin | | FOG: Predicted E3 ubiquitin |
| | Degradation | | ligase |
| 190 | | | Glutathione peroxidase |
| 191 | | | Iron/ascorbate family |
| | | | oxidoreductases |
| 192 | | | Serine carboxypeptidases |
| 193 | | | |
| 194 | | | |
| 195 | | | Glutamate decarboxylase and |
| | | | related proteins |
| 196 | | | Acyl-CoA synthetase |
| 197 | | | |
| 198 | | | Long-chain acyl-CoA |
| | | | synthetases (AMP-forming) |
| 199 | | | GPI-alpha- |
| | | | mannosyltransferase III |
| | | | (GPI10/PIG-B) involved in |
| | | | glycosylphosphatidylinositol |
| | | | anchor biosynthesis |
| 200 | | | |
| 201 | | | |
| 202 | | | |
| 203 | | | |
| 204 | | | |
| 205 | | | Beta-galactosidase |
| 206 | | | |
| 207 | | | |
| 208 | | | |
| 209 | | | Ubiquitin-protein ligase |
| 210 | | | |
| 211 | | | Chitinase |
| 212 | Transcription Factor | Basic Helix- | |
| | | Loop-Helix | |
| | | (bHLH) | |
| 213 | | | |
| 214 | | | |
| 215 | | | FKBP-type peptidyl-prolyl cis- |
| | | | trans isomerase |
| 216 | | | Haloacid dehalogenase-like |
| | | | hydrolase |
| 217 | Ethylene | | Iron/ascorbate family |
| | Biosynthesis | | oxidoreductases |
| 218 | | | |
| 219 | | | |
| 220 | Transcription Factor | Heat Shock | Heat shock transcription |
| | | | factor |
| 221 | | | |
| 222 | | | |
| 223 | | | |
| 224 | | | |
| 225 | | | |
| 226 | | | |
| 227 | Ethylene | | Iron/ascorbate family |
| | Biosynthesis | | oxidoreductases |
| 228 | Protein Kinase | | |
| 229 | | | |
| 230 | | | |
| 231 | | | O-methyltransferase |
| 232 | | | Serine carboxypeptidases |
| 233 | Transcription Factor | Basic Helix- | |
| | | Loop-Helix | |
| | | (bHLH) | |
| 234 | | | |
| 235 | | | |
| 236 | Hormone Induced | | |
| 237 | Transcription Factor | Basic Helix- | |
| | | loop-Helix | |
| | | (bHLH) | |
| 238 | Transcription Factor | NAC Domain | |
| 239 | | | Homocysteine S- |
| | | | methyltransferase |
| 240 | | | Ca2+/H+ antiporter VCX1 |
| | | | and related proteins |
| 241 | Transcription Factor | Heat Shock | Heat shock transcription |
| | | | factor |
| 242 | | | |
| 243 | | | |
| 244 | | | Glutaredoxin-related protein |
| 245 | | | NADH:flavin |
| | | | oxidoreductase/12- |
| | | | oxophytodienoate reductase |
| 246 | Transcription Factor | MYB | |
| 247 | | | Iron/ascorbate family |
| | | | oxidoreductases |
| 248 | | | Serine carboxypeptidases |
| 249 | | | |
| 250 | | | Glycine cleavage system H |
| | | | protein (lipoate-binding) |
| 251 | | | Gamma-glutamyl hydrolase |
| 252 | | | |
| 253 | | | |
| 254 | | | |
| 255 | | | |
| 256 | Transcription Factor | Basic Leucine | |
| | | Zipper (bZIP) | |
| 257 | Storage Protein | | Reticulon |
| 258 | | | Soluble epoxide hydrolase |
| 259 | | | |
| 260 | | | |
| 261 | Unclassified | | Lanthionine synthetase C-like |
| | | | protein 1 |
| 262 | | | |
| 263 | | | |
| 264 | | | |
| 265 | | | Glyoxylate/hydroxypyruvate |
| | | | reductase (D-isomer-specific |
| | | | 2-hydroxy acid |
| | | | dehydrogenase superfamily) |
| 266 | Protein Phosphatase | | |
| 267 | | | Haloacid dehalogenase-like |
| | | | hydrolase |
| 268 | Protein Phosphatase | | Serine/threonine protein |
| | | | phosphatase |
| 269 | | | |
| 270 | Storage Protein | | |
| 271 | Transcription Factor | AP2/EREBP | |
| 272 | | | Predicted metallothionein |
| 273 | Protein Phosphatase | | Serine/threonine protein |
| | | | phosphatase |
| 274 | | | |
| 275 | | | |
| 276 | | | Uncharacterized conserved |
| | | | protein |
| 277 | | | |
| | | | Transporter, ABC superfamily |
| | | | (Breast cancer resistance |
| | | | protein) |
| 278 | | | |
| 279 | Transcription Factor | Basic Helix- | |
| | | Loop-Helix | |
| | | (bHLH) | |
| 280 | | | |
| 281 | Transcription Factor | Basic Leucine | |
| | | Zipper (bZIP) | |
| 282 | | | |
| 283 | | | |
| 284 | Transcription Factor | Basic Helix- | |
| | | Loop-Helix | |
| | | (bHLH) | |
| 285 | | | Aquaporin (major intrinsic |
| | | | protein family) |
| 286 | | | |
| 287 | Amino Acid | | Arginase family protein |
| | Biosynthesis/Metabolism | | |
| 288 | | | Aldehyde dehydrogenase |
| 289 | | | Asparaginyl peptidases |
| 290 | | | Beta, beta-carotene 15,15′- |
| | | | dioxygenase and related |
| | | | enzymes |
| 291 | | | |
| 292 | | | Alcohol dehydrogenase, class V |
| 293 | | | |
| 294 | | | |
| 295 | | | |
| 296 | | | |
| 297 | | | Aquaporin (major intrinsic |
| | | | protein family) |
| 298 | | | |
| 299 | Transcription Factor | NAC Domain | |
| 300 | | | Selenium-binding protein |
| 301 | Protein Phosphatase | | Serine/threonine protein |
| | | | phosphatase |
| 302 | Transcription Factor | CCAAT | CCAAT-binding factor, |
| | | | subunit A (HAP3) |
| 303 | | | |
| 304 | | | Transporter, ABC superfamily |
| | | | (Breast cancer resistance |
| | | | protein) |
| 305 | | | |
| 306 | Transcription Factor | Zinc Finger | |
| 307 | | | |
| 308 | | | Flavonol reductase/cinnamoyl- |
| | | | CoA reductase |
| 309 | Transcription Factor | NAC Domain | |
| 310 | | | |
| 311 | Transcription Factor | Basic Helix- | |
| | | Loop-Helix | |
| | | (bHLH) | |
| 312 | | | Kynurenine 3- |
| | | | monooxygenase and related |
| | | | flavoprotein monooxygenases |
| 313 | | | |
| 314 | | | |
| 315 | | | |
| 316 | | | |
| 317 | | | Phenylalanine and histidine |
| | | | ammonia-lyase |
| 318 | | | |
| 319 | Storage Protein | | Uncharacterized conserved |
| | | | protein |
| 320 | | | Tomosyn and related SNARE- |
| | | | interacting proteins |
| 321 | | | Predicted haloacid |
| | | | dehalogenase-like hydrolase |
| 322 | Protein Phosphatase | | |
| 323 | | | |
| 324 | Protein Phosphatase | | |
| 325 | | | |
| 326 | | | |
| 327 | | | |
| 328 | | | |
| 329 | | | Tomosyn and related SNARE- |
| | | | interacting proteins |
| 330 | | | |
| 331 | | | |
| 332 | | | |
| 333 | | | |
| 334 | | | |
| 335 | | | Ubiquitin-protein ligase |
| 336 | | | |
| 337 | | | |
| 338 | | | |
| 339 | | | Glutathione S-transferase |
| 340 | Protein Phosphatase | | Serine/threonine protein |
| | | | phosphatase |
| 341 | | | H+/oligopeptide symporter |
| 342 | | | |
| 343 | | | |
| 344 | | | |
| 345 | | | |
| 346 | | | 5′-AMP-activated protein |
| | | | kinase, gamma subunit |
| 347 | | | |
| 348 | | | Beta, beta-carotene 15,15′- |
| | | | dioxygenase and related |
| | | | enzymes |
| 349 | | | |
| 350 | | | |
| 351 | Transcription Factor | Homeodomain | Transcription factor HEX, |
| | | | contains HOX and HALZ |
| | | | domains |
| 352 | | | Flavonol reductase/cinnamoyl- |
| | | | CoA reductase |
| 353 | | | 5′-AMP-activated protein |
| | | | kinase, gamma subunit |
| 354 | | | Flavonol reductase/cinnamoyl- |
| | | | CoA reductase |
| 355 | Storage Protein | | |
| 356 | | | |
| 357 | | | |
| 358 | Unclassified | | Phosphatidylethanolamine |
| | | | binding protein |
| 359 | | | Thioredoxin, nucleoredoxin |
| | | | and related proteins |
| 360 | | | 5′-AMP-activated protein |
| | | | kinase, gamma subunit |
| 361 | | | 5′-AMP-activated protein |
| | | | kinase, gamma subunit |
| 362 | Transcription Factor | CCAAT | CCAAT-binding factor, |
| | | | subunit B (HAP2) |
| 363 | | | |
| 364 | | | Permease of the major |
| | | | facilitator superfamily |
| 365 | | | Fumarase |
| 366 | | | |
| 367 | | | |
| 368 | | | |
| 369 | | | |
| 370 | | | Thioredoxin, nucleoredoxin |
| | | | and related proteins |
| 371 | | | |
| 372 | | | Thioredoxin, nucleoredoxin |
| | | | and related proteins |
| 373 | Stress Induced | | |
| 374 | Storage Protein | | Uncharacterized conserved |
| | | | protein |
| 375 | Storage Protein | | Uncharacterized conserved |
| | | | protein |

TABLE 4

| | | Fold | Soybean_Gen | |
|---|---|---|---|---|
| ID | Gene Name | Change | Bank_Acc | Functional Category |
| 1 | Gma.10892.1.S1_a_at | 14.15 | BG839570 | Metabolism |
| 2 | Gma.10921.1.S1_s_at | 8.989 | BQ611250 | No Homology to Known |
| | | | | Proteins |
| 3 | Gma.12356.1.A1_at | 8.807 | CD397798 | Unclassified - Hypothetical |
| | | | | Protein NOT Supported by |
| | | | | cDNA |
| 4 | GmaAffx.42583.1.A1_at | 8.466 | BG154898 | Transporter |
| 5 | Gma.5689.3.S1_s_at | 8.444 | BE657645 | Protein Destination & Storage |
| 6 | GmaAffx.14016.1.S1_at | 8.082 | BE824450 | No Homology to Known |
| | | | | Proteins |
| 7 | GmaAffx.9605.1.S1_at | 5.481 | BM091603 | No Homology to Known |
| | | | | Proteins |
| 8 | Gma.964.1.A1_at | 5.456 | BE824146 | Metabolism |
| 9 | Gma.4058.1.S1_at | 5.299 | AW310338 | Transporter |
| 10 | Gma.17789.1.S1_at | 5.221 | BG511704 | Unclassified - Hypothetical |
| | | | | Protein Supported by cDNA |
| 11 | Gma.1221.1.S1_s_at | 5.045 | L23854.1 | Metabolism |
| 11 | Gma.1221.1.S1_s_at | 5.045 | L23854.1 | Metabolism |
| 11 | Gma.1221.1.S1_s_at | 5.045 | L23854.1 | Metabolism |
| 12 | GmaAffx.19116.1.S1_at | 4.956 | BI968419 | Signal Transduction |
| 13 | GmaAffx.9866.1.S1_at | 4.784 | BM524432 | Metabolism |
| 14 | Gma.5139.1.S1_at | 4.589 | AF243376.1 | Secondary Metabolism |
| 15 | Gma.8416.1.S1_at | 3.823 | U13987.1 | Metabolism |
| 15 | Gma.8416.1.S1_at | 3.823 | U13987.1 | Metabolism |
| 15 | Gma.8416.1.S1_at | 3.823 | U13987.1 | Metabolism |
| 16 | Gma.6266.1.A1_at | 3.609 | CD413418 | Unclassified -Protein with |
| | | | | Unknown Function |
| 17 | GmaAffx.3905.3.S1_at | 3.456 | BG047142 | Unclassified -Protein with |
| | | | | Unknown Function |
| 18 | Gma.7332.1.S1_at | 3.119 | BI945636 | Unclassified - Hypothetical |
| | | | | Protein Supported by cDNA |
| 19 | Gma.4701.2.A1_a_at | 2.971 | BM526048 | Energy |
| 20 | GmaAffx.3369.1.S1_at | 2.835 | BU548983 | Cell Structure |
| 21 | GmaAffx.67477.2.S1_at | 2.756 | AW509184 | Cell Structure |
| 22 | Gma.3581.2.S1_a_at | 2.671 | CD398783 | Unclassified - Hypothetical |
| | | | | Protein NOT Supported by |
| | | | | cDNA |
| 23 | GmaAffx.90960.1.A1_s_at | 2.664 | CF806685 | Signal Transduction |
| 24 | GmaAffx.53845.1.S1_at | 2.63 | BG406552 | Transporter |
| 25 | Gma.17321.1.S1_at | 2.572 | AW508844 | Metabolism |
| 26 | Gma.7301.1.S1_at | 2.472 | BM528250 | Disease & Defense |
| 27 | GmaAffx.73185.1.S1_at | 2.442 | BI701903 | No Homology to Known |
| | | | | Proteins |
| 28 | Gma.3639.1.S1_at | 2.322 | AB083030.1 | Protein Destination & Storage |
| 29 | Gma.184.1.S1_at | 2.279 | AW101261 | Unclassified - Hypothetical |
| | | | | Protein Supported by cDNA |
| 30 | GmaAffx.62542.1.S1_at | 2.266 | BE659136 | Unclassified - Hypothetical |
| | | | | Protein NOT Supported by |
| | | | | cDNA |
| 31 | Gma.7608.1.S1_at | 2.247 | BU549027 | Unclassified - Hypothetical |
| | | | | Protein NOT Supported by |
| | | | | cDNA |
| 32 | GmaAffx.6571.1.S1_at | 2.238 | CD403804 | Metabolism |
| 33 | GmaAffx.40114.1.A1_at | 2.199 | BE661587 | Unclassified - Hypothetical |
| | | | | Protein Supported by cDNA |
| 34 | Gma.16521.1.S1_at | 2.185 | BE609300 | Unclassified - Hypothetical |
| | | | | Protein Supported by cDNA |
| 35 | Gma.16832.1.S1_s_at | 2.168 | L22029.1 | No Homology to Known |
| | | | | Proteins |
| 36 | GmaAffx.90947.1.S1_at | 2.161 | CF806672 | Unclassified -Protein with |
| | | | | Unknown Function |
| 37 | Gma.17816.1.A1_at | 2.151 | BI970976 | Disease & Defense |
| 38 | Gma.5483.1.S1_s_at | 2.144 | CD414581 | Transcription |
| 39 | GmaAffx.86059.1.S1_at | 2.135 | BF598759 | No Homology to Known |
| | | | | Proteins |
| 40 | GmaAffx.934.1.S1_at | 2.132 | BG046802 | No Homology to Known |
| | | | | Proteins |
| 41 | Gma.5124.1.S1_at | 2.128 | BI971079 | Protein Destination & Storage |
| 42 | Gma.4374.1.S1_at | 2.08 | BM178538 | Metabolism |
| 43 | GmaAffx.6.1.S1_at | 2.07 | AW348562 | No Homology to Known |
| | | | | Proteins |
| 44 | Gma.12212.1.A1_s_at | 2.053 | CD396776 | No Homology to Known |
| | | | | Proteins |
| 45 | Gma.3136.1.S1_at | 2.04 | BG839241 | Metabolism |
| 46 | Gma.2194.1.S1_at | 2.038 | BE657352 | Unclassified - Hypothetical |
| | | | | Protein Supported by cDNA |
| 47 | Gma.7474.1.A1_at | 2.009 | BU547968 | Unclassified - Hypothetical |
| | | | | Protein Supported by cDNA |
| 48 | Gma.12641.1.A1_at | 0.5 | CD393495 | No Homology to Known |
| | | | | Proteins |
| 49 | GmaAffx.32345.1.A1_s_at | 0.499 | BI970277 | Cell Structure |
| 50 | Gma.16910.1.S1_at | 0.498 | BU549219 | Unclassified -Protein with |
| | | | | Unknown Function |
| 51 | Gma.11665.2.A1_at | 0.498 | BU545959 | No Homology to Known |
| | | | | Proteins |
| 52 | GmaAffx.9400.2.S1_at | 0.497 | BI945484 | Signal Transduction |
| 52 | GmaAffx.9400.2.S1_at | 0.497 | BI945484 | Signal Transduction |
| 53 | Gma.2548.1.S1_at | 0.496 | BE821472 | Unclassified - Hypothetical |
| | | | | Protein Supported by cDNA |
| 54 | Gma.5929.1.S1_at | 0.496 | BQ610446 | No Homology to Known |
| | | | | Proteins |
| 55 | GmaAffx.60958.1.S1_at | 0.494 | BG551038 | Transcription |
| 56 | GmaAffx.37484.1.S1_at | 0.494 | BG651852 | Unclassified -Protein with |
| | | | | Unknown Function |
| 57 | GmaAffx.81585.1.S1_at | 0.493 | BM187887 | Unclassified - Hypothetical |
| | | | | Protein NOT Supported by |
| | | | | cDNA |
| 58 | GmaAffx.78599.1.A1_at | 0.493 | CA784082 | No Homology to Known |
| | | | | Proteins |
| 59 | GmaAffx.43774.1.A1_at | 0.492 | AW349173 | Cell Structure |
| 60 | Gma.1704.1.S1_at | 0.492 | AW349971 | Metabolism |
| 61 | GmaAffx.3697.1.S1_at | 0.492 | BE820647 | Disease & Defense |
| 62 | GmaAffx.72479.1.S1_at | 0.491 | BG043825 | Transcription |
| 63 | GmaAffx.47228.1.S1_at | 0.491 | BU761053 | Transporter |
| 64 | Gma.15907.2.S1_at | 0.491 | BE329550 | Signal Transduction |
| 65 | PsAffx.psHB026xH01f_s_at | 0.49 | | |
| 66 | Gma.13361.1.S1_at | 0.49 | AW309932 | Unclassified -Protein with |
| | | | | Unknown Function |
| 67 | Gma.11195.1.S1_at | 0.49 | CD417406 | Transporter |
| 68 | Gma.4680.1.A1_s_at | 0.489 | AW309707 | Unclassified -Protein with |
| | | | | Unknown Function |
| 69 | GmaAffx.84795.1.S1_at | 0.489 | BG046191 | Unclassified - Hypothetical |
| | | | | Protein NOT Supported by |
| | | | | cDNA |
| 70 | Gma.9013.1.S1_at | 0.488 | AF243368.1 | Disease & Defense |
| 71 | GmaAffx.71589.1.S1_at | 0.487 | BE020161 | Cell Growth & Division |
| 72 | Gma.8946.1.S1_at | 0.487 | BQ453813 | Transcription |
| 73 | GmaAffx.93189.1.S1_at | 0.487 | CF808914 | Metabolism |
| 74 | GmaAffx.75219.1.S1_at | 0.486 | BE020773 | Unclassified - Hypothetical |
| | | | | Protein NOT Supported by |
| | | | | cDNA |
| 75 | Gma.10896.3.A1_at | 0.486 | BI316837 | Metabolism |
| 76 | GmaAffx.83564.1.S1_at | 0.485 | BU578309 | No Homology to Known |
| | | | | Proteins |
| 77 | GmaAffic.40086.1.S1_at | 0.485 | BE659815 | Unclassified - Hypothetical |
| | | | | Protein Supported by cDNA |
| 78 | Gma.6129.2.S1_s_at | 0.484 | AW186006 | Protein Synthesis |
| 79 | Gma.1715.1.S1_at | 0.483 | BE191618 | Metabolism |
| 80 | GmaAffx.72986.1.S1_at | 0.483 | BI317536 | Metabolism |
| 81 | GmaAffx.89049.1.S1_at | 0.483 | CK606168 | Intracellular Traffic |
| 82 | GmaAffx.64807.1.S1_at | 0.483 | AW201972 | No Homology to Known |
| | | | | Proteins |
| 83 | GmaAffx.26967.1.S1_at | 0.482 | BE022958 | Unclassified - Hypothetical |
| | | | | Protein Supported by cDNA |
| 84 | Gma.2540.2.S1_s_at | 0.482 | BI787366 | No Homology to Known |
| | | | | Proteins |
| 85 | GmaAffx.28578.1.S1_at | 0.482 | BQ094224 | Disease & Defense |
| 86 | GmaAffx.90331.1.S1_s_at | 0.482 | CF806056 | Disease & Defense |
| 87 | GmaAffx.68754.1.S1_at | 0.481 | BE474746 | Secondary Metabolism |
| 88 | Gma.5889.1.S1_at | 0.481 | BQ610202 | Unclassified - Hypothetical |
| | | | | Protein NOT Supported by |
| | | | | cDNA |
| 89 | Gma.1137.2.S1_x_at | 0.481 | CA852403 | Metabolism |
| 90 | Gma.3591.1.S1_x_at | 0.481 | BE440266 | Secondary Metabolism |
| 91 | Gma.7970.1.S1_at | 0.481 | BI699189 | No Homology to Known |
| | | | | Proteins |
| 92 | GmaAffx.28539.1.S1_at | 0.481 | BQ080184 | Disease & Defense |
| 93 | GmaAffx.61311.1.S1_at | 0.48 | CA936951 | Transporter |
| 94 | GmaAffx.30428.1.S1_at | 0.48 | BE824410 | Protein Destination & Storage |
| 95 | Gma.16585.1.S1_x_at | 0.48 | CA850597 | Metabolism |
| 96 | Gma.8160.1.S1_at | 0.48 | BI787214 | Unclassified - Hypothetical |
| | | | | Protein NOT Supported by |
| | | | | cDNA |
| 97 | GmaAffx.87670.1.S1_at | 0.48 | BQ452640 | Unclassified - Hypothetical |
| | | | | Protein NOT Supported by |
| | | | | cDNA |
| 98 | Gma.728.1.A1_at | 0.479 | BE658779 | No Homology to Known |
| | | | | Proteins |
| 99 | Gma.4120.1.A1_at | 0.479 | AW310714 | Unclassified -Protein with |
| | | | | Unknown Function |
| 100 | Gma.6630.1.S1_s_at | 0.478 | BG652851 | Unclassified - Hypothetical |
| | | | | Protein Supported by cDNA |
| 101 | GmaAffx.81584.1.S1_at | 0.478 | BI426969 | Disease & Defense |
| 102 | Gma.7040.1.A1_at | 0.478 | BE346424 | Transcription |
| 103 | GmaAffx.69076.1.S1_at | 0.477 | BM525610 | Unclassified - Hypothetical |
| | | | | Protein NOT Supported by |
| | | | | cDNA |
| 104 | GmaAffx.53099.1.S1_at | 0.477 | BG043340 | Unclassified - Hypothetical |
| | | | | Protein NOT Supported by |
| | | | | cDNA |
| 105 | Gma.8240.1.S1_at | 0.476 | BM092761 | Secondary Metabolism |
| 106 | GmaAffx.30665.1.A1_at | 0.476 | BQ453743 | Signal Transduction |
| 107 | Gma.16526.1.S1_at | 0.475 | CD407998 | Protein Destination & Storage |
| 108 | GmaAffx.16207.1.S1_at | 0.475 | BE821188 | Cell Structure |
| 109 | GmaAffx.8712.1.S1_s_at | 0.474 | AW349107 | Disease & Defense |
| 110 | Gma.1951.2.S1_a_at | 0.474 | CA783485 | Protein Destination & Storage |
| 111 | GmaAffx.46684.1.S1_at | 0.474 | BQ081034 | Signal Transduction |
| 112 | Gma.13000.1.S1_at | 0.474 | CD415111 | Unclassified - Hypothetical |
| | | | | Protein Supported by cDNA |
| 113 | Gma.9205.1.S1_at | 0.473 | BM528605 | Unclassified - Hypothetical |
| | | | | Protein Supported by cDNA |
| 114 | Gma.13035.1.S1_at | 0.473 | CD396536 | No Homology to Known |
| | | | | Proteins |
| 115 | Gma.285.1.S1_at | 0.472 | BG726992 | Cell Growth & Division |
| 116 | Gma.10824.1.S1_at | 0.472 | BE824257 | Unclassified - Hypothetical |
| | | | | Protein Supported by cDNA |
| 117 | GmaAffx.91641.1.S1_s_at | 0.472 | CF807366 | Disease & Defense |
| 118 | Gma.4610.1.S1_s_at | 0.472 | BM522856 | No Homology to Known |
| | | | | Proteins |
| 119 | GmaAffx.86136.1.S1_at | 0.471 | AI440673 | Unclassified -Protein with |
| | | | | Unknown Function |
| 120 | Gma.6498.1.A1_at | 0.471 | BE824396 | Unclassified - Hypothetical |
| | | | | Protein Supported by cDNA |
| 121 | GmaAffx.92136.1.S1_s_at | 0.471 | CF807451 | Signal Transduction |
| 122 | GmaAffx.26320.1.S1_at | 0.469 | BE440416 | Unclassified - Hypothetical |
| | | | | Protein Supported by cDNA |
| 123 | Gma.1332.1.S1_s_at | 0.468 | BI970666 | Secondary Metabolism |
| 124 | Gma.16477.1.S1_at | 0.467 | BE824134 | No Homology to Known |
| | | | | Proteins |
| 125 | GmaAffx.4936.1.S1_at | 0.467 | AW666270 | Transporter |
| 126 | Gma.7824.1.S1_at | 0.465 | BU550492 | Metabolism |
| 127 | Gma.3283.1.S1_at | 0.465 | BQ298733 | No Homology to Known |
| | | | | Proteins |
| 128 | GmaAffx.89049.1.S1_s_at | 0.464 | CK606168 | Intracellular Traffic |
| 129 | Gma.10026.1.S1_at | 0.464 | BU546301 | Unclassified -Protein with |
| | | | | Unknown Function |
| 130 | Gma.16896.1.A1_at | 0.462 | CD416188 | No Homology to Known |
| | | | | Proteins |
| 131 | Gma.1980.1.A1_at | 0.462 | BM520466 | Signal Transduction |
| 132 | GmaAffx.49130.1.S1_at | 0.462 | BE555035 | No Homology to Known |
| | | | | Proteins |
| 133 | GmaAffx.14141.1.A1_at | 0.462 | BI973009 | No Homology to Known |
| | | | | Proteins |
| 134 | Gma.1528.1.S1_at | 0.461 | CD403623 | Transcription |
| 135 | GmaAffx.86392.1.S1_at | 0.46 | AI736754 | Signal Transduction |
| 136 | Gma.10581.1.S1_at | 0.46 | AW396439 | Unclassified - Hypothetical |
| | | | | Protein Supported by cDNA |
| 137 | GmaAffx.35729.1.S1_s_at | 0.46 | BE806047 | Unclassified - Hypothetical |
| | | | | Protein Supported by cDNA |
| 138 | GmaAffx.65419.1.S1_at | 0.459 | CD405955 | Signal Transduction |
| 139 | Gma.412.1.S1_at | 0.459 | BE607835 | Protein Destination & Storage |
| 140 | GmaAffx.90069.1.S1_s_at | 0.459 | CF805794 | Unclassified - Hypothetical |
| | | | | Protein Supported by cDNA |
| 141 | Gma.1839.2.S1_a_at | 0.459 | AW507949 | Metabolism |
| 142 | Gma.17288.1.A1_at | 0.458 | AW310692 | No Homology to Known |
| | | | | Proteins |
| 143 | GmaAffx.58712.1.S1_at | 0.458 | BF325034 | Transporter |
| 144 | Gma.850.1.S1_at | 0.458 | BE822050 | Unclassified - Hypothetical |
| | | | | Protein NOT Supported by |
| | | | | cDNA |
| 145 | GmaAffx.91731.1.S1_s_at | 0.458 | CF807456 | Secondary Metabolism |
| 146 | GmaAffx.74868.1.S1_at | 0.457 | CA820573 | No Homology to Known |
| | | | | Proteins |
| 147 | GmaAffx.29254.1.S1_at | 0.456 | AW760361 | Transcription |
| 148 | Gma.4394.1.S1_a_at | 0.456 | CD409273 | Transcription |
| 149 | Gma.17759.1.S1_at | 0.456 | BU927041 | Transcription |
| 150 | Gma.10393.1.S1_at | 0.455 | BE804608 | Unclassified - Hypothetical |
| | | | | Protein NOT Supported by |
| | | | | cDNA |
| 151 | Gma.3591.1.S1_a_at | 0.455 | BE440266 | Secondary Metabolism |
| 152 | Gma.1573.2.S1_a_at | 0.454 | BI974098 | Metabolism |
| 153 | GmaAffx.90789.1.S1_s_at | 0.454 | CF806514 | Unclassified -Protein with |
| | | | | Unknown Function |
| 154 | GmaAffx.67113.1.S1_at | 0.454 | BE329541 | No Homology to Known |
| | | | | Proteins |
| 155 | GmaAffx.51444.1.S1_at | 0.453 | BM178639 | Secondary Metabolism |
| 156 | Gma.1068.1.S1_at | 0.453 | L19360.1 | Signal Transduction |
| 157 | GmaAffx.44471.1.A1_at | 0.452 | CA851833 | Unclassified - Hypothetical |
| | | | | Protein NOT Supported by |
| | | | | cDNA |
| 158 | GmaAffx.9145.1.S1_s_at | 0.452 | BU763814 | Disease & Defense |
| 159 | Gma.3531.1.S1_s_at | 0.452 | BI498803 | Unclassified - Hypothetical |
| | | | | Protein Supported by cDNA |
| 160 | GmaAffx.84209.1.S1_at | 0.452 | BQ610588 | Signal Transduction |
| 161 | GmaAffx.494.1.S1_at | 0.452 | AI938330 | Cell Structure |
| 162 | Gma.16940.1.S1_at | 0.451 | AW310039 | Transcription |
| 163 | GmaAffx.32011.1.S1_at | 0.449 | BI701198 | No Homology to Known |
| | | | | Proteins |
| 164 | Gma.3963.2.S1_at | 0.449 | BE608192 | Unclassified - Hypothetical |
| | | | | Protein NOT Supported by |
| | | | | cDNA |
| 165 | Gma.1263.1.S1_at | 0.449 | AY144180.1 | Metabolism |
| 166 | GmaAffx.72346.1.S1_at | 0.448 | BF424359 | Unclassified - Hypothetical |
| | | | | Protein NOT Supported by |
| | | | | cDNA |
| 167 | Gma.10144.2.S1_at | 0.447 | BU080819 | Metabolism |
| 168 | GmaAffx.82263.1.S1_s_at | 0.447 | AW568454 | No Homology to Known |
| | | | | Proteins |
| 169 | Gma.12068.1.A1_at | 0.446 | BU547739 | Transporter |
| 170 | GmaAffx.83880.1.A1_at | 0.446 | BE822640 | Disease & Defense |
| 171 | GmaAffx.9040.1.S1_at | 0.446 | AW101738 | Unclassified - Hypothetical |
| | | | | Protein Supported by cDNA |
| 172 | Gma.3893.3.S1_at | 0.446 | BG651854 | Unclassified - Hypothetical |
| | | | | Protein Supported by cDNA |
| 173 | GmaAffx.92184.1.S1_s_at | 0.445 | CF807909 | Unclassified -Protein with |
| | | | | Unknown Function |
| 174 | GmaAffx.45957.2.S1_at | 0.445 | BI974135 | No Homology to Known |
| | | | | Proteins |
| 175 | Gma.6264.1.S1_at | 0.444 | BQ612558 | Unclassified - Hypothetical |
| | | | | Protein Supported by cDNA |
| 176 | GmaAffx.86514.1.S1_at | 0.444 | BG790017 | Transcription |
| 177 | GmaAffx.93182.1.S1_s_at | 0.443 | CF808907 | Secondary Metabolism |
| 178 | GmaAffx.58073.1.S1_at | 0.443 | CA852105 | Unclassified - Hypothetical |
| | | | | Protein Supported by cDNA |
| 179 | Gma.11028.1.S1_s_at | 0.443 | BE823084 | Metabolism |
| 180 | Gma.6550.2.S1_s_at | 0.442 | BM086044 | Metabolism |
| 181 | GmaAffx.91266.1.S1_s_at | 0.442 | CF806991 | Unclassified - Hypothetical |
| | | | | Protein Supported by cDNA |
| 182 | Gma.5331.1.S1_a_at | 0.442 | BI892702 | Transcription |
| 183 | GmaAffx.18880.1.S1_at | 0.442 | BM094235 | Signal Transduction |
| 184 | Gma.3579.1.S1_at | 0.442 | AW349799 | Unclassified - Hypothetical |
| | | | | Protein Supported by cDNA |
| 185 | Gma.14471.4.S1_x_at | 0.441 | BQ081409 | Transporter |
| 186 | GmaAffx.65140.1.S1_at | 0.441 | BI700031 | No Homology to Known |
| | | | | Proteins |
| 187 | Gma.1895.1.A1_at | 0.44 | AW309584 | Protein Destination & Storage |
| 188 | Gma.16528.1.S1_at | 0.44 | CD398706 | Unclassified - Hypothetical |
| | | | | Protein Supported by cDNA |
| 189 | Gma.10950.1.S1_at | 0.44 | BI699798 | Unclassified - Hypothetical |
| | | | | Protein Supported by cDNA |
| 190 | Gma.15996.1.S1_at | 0.439 | BQ454193 | Unclassified - Hypothetical |
| | | | | Protein Supported by cDNA |
| 191 | GmaAffx.1785.1.S1_at | 0.439 | BF325331 | Transcription |
| 192 | GmaAffx.55953.1.S1_at | 0.439 | BQ629142 | Unclassified - Hypothetical |
| | | | | Protein NOT Supported by |
| | | | | cDNA |
| 193 | GmaAffx.93553.1.S1_s_at | 0.439 | CF809278 | Metabolism |
| 194 | GmaAffx.74194.1.S1_at | 0.438 | BI968058 | Metabolism |
| 195 | GmaAffx.87793.2.S1_at | 0.437 | BQ740671 | Signal Transduction |
| 196 | GmaAffx.36470.1.A1_at | 0.436 | BU550552 | Unclassified - Hypothetical |
| | | | | Protein Supported by cDNA |
| 197 | GmaAffx.42856.1.S1_at | 0.435 | BM567981 | Transcription |
| 198 | GmaAffx.1301.78.S1_s_at | 0.435 | AW394497 | No Homology to Known |
| | | | | Proteins |
| 199 | GmaAffx.29951.2.S1_at | 0.434 | AW100899 | Metabolism |
| 200 | Gma.11334.1.S1_a_at | 0.433 | BI967327 | Cell Structure |
| 201 | Gma.5559.1.S1_s_at | 0.433 | BM121524 | Unclassified - Hypothetical |
| | | | | Protein Supported by cDNA |
| 202 | GmaAffx.92230.1.A1_s_at | 0.432 | CF807955 | Disease & Defense |
| 203 | GmaAffx.87679.1.S1_at | 0.431 | BM523734 | Metabolism |
| 204 | Gma.398.1.A1_at | 0.43 | AW350679 | Metabolism |
| 205 | Gma.4665.1.A1_at | 0.43 | AW309815 | No Homology to Known |
| | | | | Proteins |
| 206 | GmaAffx.27331.1.S1_at | 0.428 | BM520297 | Unclassified - Hypothetical |
| | | | | Protein NOT Supported by |
| | | | | cDNA |
| 207 | GmaAffx.34975.1.S1_at | 0.428 | BU547817 | Unclassified - Hypothetical |
| | | | | Protein Supported by cDNA |
| 208 | Gma.10969.1.S1_a_at | 0.426 | U36191.1 | Metabolism |
| 209 | Gma.1661.1.S1_at | 0.424 | BE821382 | Metabolism |
| 210 | Gma.2080.2.S1_a_at | 0.424 | BM520916 | Secondary Metabolism |
| 211 | GmaAffx.25612.1.S1_at | 0.424 | AW317577 | No Homology to Known |
| | | | | Proteins |
| 212 | GmaAffx.50677.1.S1_s_at | 0.423 | BI971744 | No Homology to Known |
| | | | | Proteins |
| 213 | GmaAffx.89624.1.A1_s_at | 0.422 | CK606100 | Transcription |
| 214 | Gma.8514.1.S1_at | 0.422 | CA851666 | Protein Destination & Storage |
| 215 | GmaAffx.91328.1.S1_at | 0.421 | CF807053 | Unclassified - Hypothetical |
| | | | | Protein Supported by cDNA |
| 216 | GmaAffx.47891.1.S1_s_at | 0.42 | BQ785880 | Cell Structure |
| 217 | Gma.6278.1.S1_at | 0.419 | BQ612647 | Unclassified - Hypothetical |
| | | | | Protein Supported by cDNA |
| 218 | GmaAffx.487.1.S1_at | 0.419 | BG511495 | Transporter |
| 219 | GmaAffx.84206.1.S1_at | 0.418 | BU760571 | Transcription |
| 220 | Gma.8058.1.A1_at | 0.417 | BM271479 | Signal Transduction |
| 221 | GmaAffx.47230.2.S1_at | 0.417 | BG652486 | Transporter |
| 222 | Gma.17985.2.S1_at | 0.417 | BE802263 | Metabolism |
| 223 | Gma.11277.2.S1_at | 0.416 | BI701238 | Unclassified - Hypothetical |
| | | | | Protein NOT Supported by |
| | | | | cDNA |
| 224 | Gma.5400.1.S1_at | 0.416 | BM093775 | No Homology to Known |
| | | | | Proteins |
| 225 | GmaAffx.33121.1.S1_at | 0.416 | BQ611246 | Unclassified - Hypothetical |
| | | | | Protein Supported by cDNA |
| 226 | Gma.6550.2.S1_x_at | 0.413 | BM086044 | Metabolism |
| 227 | GmaAffx.39703.1.S1_at | 0.413 | BE023656 | Protein Destination & Storage |
| 228 | GmaAffx.15222.1.S1_at | 0.413 | BU550700 | Unclassified - Hypothetical |
| | | | | Protein NOT Supported by |
| | | | | cDNA |
| 229 | GmaAffx.20146.1.S1_at | 0.413 | BE824274 | Unclassified -Protein with |
| | | | | Unknown Function |
| 230 | Gma.5586.2.S1_at | 0.412 | CD398384 | Transcription |
| 231 | GmaAffx.71016.1.S1_at | 0.411 | AW234455 | Unclassified - Hypothetical |
| | | | | Protein Supported by cDNA |
| 232 | Gma.10536.1.S1_at | 0.411 | BG508300 | No Homology to Known |
| | | | | Proteins |
| 233 | GmaAffx.74160.1.S1_at | 0.411 | BQ740845 | Protein Destination & Storage |
| 234 | GmaAffx.91141.1.S1_at | 0.411 | CF806866 | Disease & Defense |
| 235 | GmaAffx.11049.1.S1_at | 0.41 | BU545140 | Unclassified -Protein with |
| | | | | Unknown Function |
| 236 | Gma.17326.1.A1_at | 0.41 | AW568254 | Unclassified - Hypothetical |
| | | | | Protein NOT Supported by |
| | | | | cDNA |
| 237 | Gma.16196.1.S1_at | 0.41 | AW350888 | Transcription |
| 238 | Gma.6288.1.S1_at | 0.41 | AW348556 | Transporter |
| 239 | GmaAffx.23289.1.S1_at | 0.409 | CD392087 | No Homology to Known |
| | | | | Proteins |
| 240 | GmaAffx.93592.1.S1_x_at | 0.409 | CF809317 | Unclassified - Hypothetical |
| | | | | Protein Supported by cDNA |
| 241 | GmaAffx.92774.1.S1_at | 0.408 | CF808124 | Cell Structure |
| 242 | GmaAffx.81851.1.S1_at | 0.408 | AW706751 | Unclassified - Hypothetical |
| | | | | Protein Supported by cDNA |
| 243 | GmaAffx.92927.1.S1_at | 0.407 | CF805992 | No Homology to Known |
| | | | | Proteins |
| 244 | GmaAffx.64794.1.S1_at | 0.407 | CA801749 | Unclassified - Hypothetical |
| | | | | Protein Supported by cDNA |
| 245 | GmaAffx.478.2.S1_at | 0.406 | BU763963 | Transcription |
| 246 | GmaAffx.24368.1.A1_at | 0.405 | BU545331 | No Homology to Known |
| | | | | Proteins |
| 247 | Gma.1394.2.S1_a_at | 0.404 | BI470370 | Metabolism |
| 248 | GmaAffx.84424.1.S1_at | 0.404 | AW311543 | Metabolism |
| 249 | GmaAffx.93373.1.S1_s_at | 0.403 | CF809098 | Unclassified - Hypothetical |
| | | | | Protein Supported by cDNA |
| 250 | GmaAffx.34449.1.S1_at | 0.403 | AW164447 | No Homology to Known |
| | | | | Proteins |
| 251 | GmaAffx.64037.1.S1_at | 0.403 | BI321162 | Metabolism |
| 252 | Gma.6709.1.S1_at | 0.402 | BE823291 | Signal Transduction |
| 253 | Gma.17015.1.S1_at | 0.402 | AW733363 | No Homology to Known |
| | | | | Proteins |
| 254 | Gma.3963.1.S1_at | 0.402 | AW202007 | Unclassified - Hypothetical |
| | | | | Protein NOT Supported by |
| | | | | cDNA |
| 255 | Gma.8113.1.A1_at | 0.401 | BU548342 | No Homology to Known |
| | | | | Proteins |
| 256 | GmaAffx.88675.1.A1_at | 0.4 | CD417704 | Unclassified - Hypothetical |
| | | | | Protein NOT Supported by |
| | | | | cDNA |
| 257 | GmaAffx.5255.1.S1_at | 0.398 | BG362929 | Transcription |
| 258 | Gma.5294.1.S1_s_at | 0.398 | BE658252 | Unclassified - Hypothetical |
| | | | | Protein Supported by cDNA |
| 259 | Gma.3776.1.S1_at | 0.398 | AW780736 | Metabolism |
| 260 | GmaAffx.44396.1.S1_at | 0.398 | BE330360 | No Homology to Known |
| | | | | Proteins |
| 261 | GmaAffx.80393.1.A1_at | 0.397 | BE658680 | No Homology to Known |
| | | | | Proteins |
| 262 | GmaAffx.82936.1.S1_at | 0.397 | AI938364 | Unclassified -Protein with |
| | | | | Unknown Function |
| 263 | Gma.6075.1.S1_a_at | 0.396 | AW317183 | Disease & Defense |
| 264 | GmaAffx.62364.1.S1_at | 0.395 | AW733764 | Protein Destination & Storage |
| 265 | GmaAffx.80900.1.S1_at | 0.393 | BE347494 | Metabolism |
| 266 | GmaAffx.79390.3.S1_at | 0.393 | BU082743 | Transcription |
| 267 | GmaAffx.4888.1.A1_at | 0.392 | BI967635 | No Homology to Known |
| | | | | Proteins |
| 268 | Gma.6727.1.S1_at | 0.39 | Y10493.1 | Metabolism |
| 269 | GmaAffx.79390.1.S1_at | 0.39 | BU550458 | Transcription |
| 270 | GmaAffx.47590.1.S1_at | 0.39 | CA853861 | Metabolism |
| 271 | GmaAffx.99.4.S1_at | 0.39 | BQ742171 | Unclassified -Protein with |
| | | | | Unknown Function |
| 272 | Gma.4822.1.S1_at | 0.388 | BI969249 | Disease & Defense |
| 273 | GmaAffx.63822.1.S1_at | 0.388 | BM520838 | Disease & Defense |
| 274 | GmaAffx.87547.2.S1_at | 0.388 | BQ080473 | Cell Structure |
| 275 | GmaAffx.78297.1.S1_at | 0.387 | BM731185 | Unclassified - Hypothetical |
| | | | | Protein Supported by cDNA |
| 276 | Gma.10827.2.S1_s_at | 0.386 | BQ612452 | Protein Destination & Storage |
| 277 | GmaAffx.82000.1.S1_at | 0.385 | BI094895 | Unclassified - Hypothetical |
| | | | | Protein Supported by cDNA |
| 278 | Gma.13800.1.A1_at | 0.384 | CD405906 | Unclassified - Hypothetical |
| | | | | Protein NOT Supported by |
| | | | | cDNA |
| 279 | GmaAffx.24451.1.A1_at | 0.384 | BU550405 | Metabolism |
| 280 | GmaAffx.85657.1.S1_s_at | 0.384 | AW185911 | Unclassified -Protein with |
| | | | | Unknown Function |
| 281 | Gma.13736.1.A1_at | 0.384 | CD404408 | Secondary Metabolism |
| 282 | GmaAffx.84338.1.S1_at | 0.384 | AI856789 | Unclassified - Hypothetical |
| | | | | Protein Supported by cDNA |
| 283 | GmaAffx.53108.1.S1_at | 0.384 | BM093748 | Signal Transduction |
| 284 | Gma.16875.1.A1_at | 0.383 | CD406723 | Metabolism |
| 285 | GmaAffx.44484.1.S1_at | 0.383 | BE475213 | Unclassified - Hypothetical |
| | | | | Protein NOT Supported by |
| | | | | cDNA |
| 286 | Gma.17070.1.S1_at | 0.382 | BE822463 | Disease & Defense |
| 287 | Gma.2376.1.S1_at | 0.382 | CA852095 | Transcription |
| 288 | Gma.1642.3.S1_s_at | 0.382 | BM891895 | Unclassified - Hypothetical |
| | | | | Protein Supported by cDNA |
| 289 | Gma.16842.1.A1_at | 0.382 | CD392652 | No Homology to Known |
| | | | | Proteins |
| 290 | Gma.2686.1.S1_s_at | 0.381 | BI970455 | Secondary Metabolism |
| 291 | GmaAffx.90570.1.S1_at | 0.381 | CF806295 | Unclassified - Hypothetical |
| | | | | Protein Supported by cDNA |
| 292 | GmaAffx.83377.2.S1_at | 0.38 | BM893101 | Transcription |
| 293 | Gma.7494.1.S1_at | 0.38 | BU548105 | Signal Transduction |
| 294 | GmaAffx.1301.68.S1_s_at | 0.378 | BI974374 | Intracellular Traffic |
| 295 | GmaAffx.15983.2.S1_s_at | 0.377 | AW279540 | Signal Transduction |
| 296 | GmaAffx.88786.1.A1_s_at | 0.376 | CK606552 | Cell Structure |
| 297 | Gma.5478.1.S1_at | 0.374 | AW350606 | Metabolism |
| 298 | Gma.6550.3.S1_s_at | 0.374 | AW234450 | Metabolism |
| 299 | Gma.1490.1.S1_at | 0.374 | AB000097.1 | Cell Structure |
| 300 | GmaAffx.11999.1.S1_at | 0.374 | AW567661 | No Homology to Known |
| | | | | Proteins |
| 301 | GmaAffx.41990.1.S1_at | 0.374 | CA936708 | No Homology to Known |
| | | | | Proteins |
| 302 | GmaAffx.66451.1.S1_at | 0.373 | AW309198 | Secondary Metabolism |
| 303 | GmaAffx.79888.1.S1_at | 0.372 | AI988123 | Signal Transduction |
| 304 | Gma.3420.1.S1_at | 0.37 | CD406082 | Unclassified - Hypothetical |
| | | | | Protein Supported by cDNA |
| 305 | GmaAffx.80098.1.S1_at | 0.37 | BG042861 | Metabolism |
| 306 | GmaAffx.1128.1.S1_at | 0.37 | AW348608 | Unclassified -Protein with |
| | | | | Unknown Function |
| 307 | Gma.12094.1.A1_at | 0.37 | CD395088 | Unclassified - Hypothetical |
| | | | | Protein Supported by cDNA |
| 308 | GmaAffx.90812.1.S1_s_at | 0.37 | CF806537 | Disease & Defense |
| 309 | GmaAffx.9926.2.S1_at | 0.368 | BF597587 | Unclassified - Hypothetical |
| | | | | Protein Supported by cDNA |
| 310 | Gma.6640.1.S1_at | 0.367 | BQ628278 | Disease & Defense |
| 311 | GmaAffx.1301.59.S1_at | 0.367 | BI974591 | Intracellular Traffic |
| 312 | GmaAffx.21041.1.A1_at | 0.367 | AW350819 | No Homology to Known |
| | | | | Proteins |
| 313 | Gma.16359.1.A1_at | 0.366 | BI970359 | Metabolism |
| 314 | Gma.1195.1.S1_s_at | 0.366 | AI442801 | Metabolism |
| 315 | GmaAffx.91205.1.S1_at | 0.365 | CF807869 | Metabolism |
| 316 | GmaAffx.93342.1.S1_s_at | 0.364 | CF809067 | Disease & Defense |
| 317 | GmaAffx.74899.1.S1_at | 0.364 | CA852377 | No Homology to Known |
| | | | | Proteins |
| 318 | GmaAffx.15613.1.S1_at | 0.364 | AI939073 | Unclassified - Hypothetical |
| | | | | Protein NOT Supported by |
| | | | | cDNA |
| 319 | GmaAffx.18953.1.S1_at | 0.363 | BM178798 | No Homology to Known |
| | | | | Proteins |
| 320 | GmaAffx.46837.1.S1_at | 0.362 | BU550919 | Secondary Metabolism |
| 321 | GmaAffx.50646.1.A1_at | 0.362 | AW310451 | Unclassified - Hypothetical |
| | | | | Protein Supported by cDNA |
| 322 | Gma.2887.2.S1_x_at | 0.362 | BI701520 | Secondary Metabolism |
| 323 | Gma.17835.1.S1_at | 0.361 | BE822840 | Metabolism |
| 324 | Gma.6719.2.S1_at | 0.361 | BG726773 | No Homology to Known |
| | | | | Proteins |
| 325 | GmaAffx.86927.1.S1_at | 0.36 | CA937162 | No Homology to Known |
| | | | | Proteins |
| 326 | Gma.3804.1.A1_at | 0.36 | AW310249 | Disease & Defense |
| 327 | GmaAffx.93189.1.S1_s_at | 0.359 | CF808914 | Metabolism |
| 328 | GmaAffx.50980.1.S1_s_at | 0.358 | BE823095 | Signal Transduction |
| 329 | Gma.821.1.S1_at | 0.357 | AF243371.1 | Metabolism |
| 330 | GmaAffx.18599.1.S1_at | 0.357 | BI787464 | Secondary Metabolism |
| 331 | Gma.5634.1.S1_at | 0.356 | BE806891 | Secondary Metabolism |
| 332 | GmaAffx.74256.1.S1_at | 0.356 | BE057293 | No Homology to Known |
| | | | | Proteins |
| 333 | Gma.2086.1.S1_at | 0.355 | AF035671.1 | Metabolism |
| 334 | Gma.73.1.S1_at | 0.354 | AF098662.1 | Protein Destination & Storage |
| 335 | GmaAffx.58125.1.A1_at | 0.353 | BE347561 | Transcription |
| 336 | Gma.12731.2.A1_s_at | 0.353 | CD395848 | Unclassified - Hypothetical |
| | | | | Protein NOT Supported by |
| | | | | cDNA |
| 337 | GmaAffx.91960.1.S1_at | 0.353 | CF807685 | Metabolism |
| 338 | GmaAffx.87793.1.S1_at | 0.353 | BI941782 | Unclassified - Hypothetical |
| | | | | Protein NOT Supported by |
| | | | | cDNA |
| 339 | GmaAffx.6790.1.S1_at | 0.352 | AI856683 | Disease & Defense |
| 340 | GmaAffx.15471.1.S1_at | 0.352 | BQ611747 | Transcription |
| 341 | GmaAffx.92192.1.S1_s_at | 0.352 | CF806406 | Disease & Defense |
| 342 | Gma.1929.1.S1_at | 0.352 | BU083650 | Transcription |
| 343 | Gma.17739.1.S1_at | 0.352 | AW310535 | Unclassified - Hypothetical |
| | | | | Protein Supported by cDNA |
| 344 | GmaAffx.59014.1.S1_s_at | 0.35 | AW733977 | No Homology to Known |
| | | | | Proteins |
| 345 | Gma.7120.2.S1_a_at | 0.35 | BG041285 | Metabolism |
| 346 | GmaAffx.478.1.S1_at | 0.348 | BE822248 | Transcription |
| 347 | GmaAffx.11892.1.S1_at | 0.348 | BE820605 | Secondary Metabolism |
| 348 | Gma.3888.2.S1_at | 0.347 | BM139532 | Unclassified - Hypothetical |
| | | | | Protein NOT Supported by |
| | | | | cDNA |
| 349 | GmaAffx.82218.1.S1_at | 0.347 | BI702052 | Secondary Metabolism |
| 350 | GmaAffx.93642.1.S1_s_at | 0.347 | CF809367 | Disease & Defense |
| 351 | Gma.6047.1.S1_s_at | 0.346 | BQ611159 | Unclassified -Protein with |
| | | | | Unknown Function |
| 352 | Gma.5634.1.S1_s_at | 0.344 | BE806891 | Secondary Metabolism |
| 353 | Gma.10969.3.S1_x_at | 0.343 | CD396552 | Metabolism |
| 354 | Gma.18006.1.S1_at | 0.343 | CF922923 | Transcription |
| 355 | GmaAffx.62195.1.S1_at | 0.343 | AW472439 | No Homology to Known |
| | | | | Proteins |
| 356 | Gma.10854.1.S1_at | 0.343 | CA802334 | No Homology to Known |
| | | | | Proteins |
| 357 | GmaAffx.30593.1.S1_at | 0.342 | BE803365 | Unclassified - Hypothetical |
| | | | | Protein Supported by cDNA |
| 358 | Gma.5258.1.S1_at | 0.341 | BE658899 | No Homology to Known |
| | | | | Proteins |
| 359 | Gma.4401.2.S1_a_at | 0.339 | BI970725 | Disease & Defense |
| 360 | GmaAffx.75740.1.S1_at | 0.338 | BG157373 | Unclassified - Hypothetical |
| | | | | Protein NOT Supported by |
| | | | | cDNA |
| 361 | GmaAffx.64261.1.A1_at | 0.338 | BI969080 | Unclassified -Protein with |
| | | | | Unknown Function |
| 362 | Gma.4981.1.A1_at | 0.337 | BI970256 | No Homology to Known |
| | | | | Proteins |
| 363 | Gma.3522.1.S1_at | 0.336 | BM140209 | Unclassified - Hypothetical |
| | | | | Protein Supported by cDNA |
| 364 | GmaAffx.30792.1.A1_at | 0.335 | BU544815 | Unclassified - Hypothetical |
| | | | | Protein Supported by cDNA |
| 365 | Gma.7437.1.S1_at | 0.332 | BU547705 | Unclassified - Hypothetical |
| | | | | Protein Supported by cDNA |
| 366 | Gma.7254.1.S1_at | 0.332 | BU546166 | Unclassified - Hypothetical |
| | | | | Protein Supported by cDNA |
| 367 | GmaAffx.91312.1.S1_s_at | 0.332 | CF806002 | Transcription |
| 368 | Gma.14075.1.S1_at | 0.331 | CD415333 | Protein Destination & Storage |
| 369 | Gma.15155.2.S1_at | 0.33 | BG789619 | Unclassified - Hypothetical |
| | | | | Protein Supported by cDNA |
| 370 | Gma.1622.1.A1_s_at | 0.33 | BU090523 | Signal Transduction |
| 371 | GmaAffx.61872.1.S1_at | 0.329 | AW102386 | Signal Transduction |
| 372 | GmaAffx.50673.2.S1_s_at | 0.329 | BI787147 | Transcription |
| 373 | GmaAffx.26868.1.S1_at | 0.328 | BF325443 | Unclassified - Hypothetical |
| | | | | Protein NOT Supported by |
| | | | | cDNA |
| 374 | GmaAffx.91185.1.S1_at | 0.327 | CF806910 | Cell Structure |
| 375 | GmaAffx.47230.1.S1_at | 0.327 | CA820051 | Transporter |
| 376 | Gma.5622.1.S1_at | 0.326 | BG363493 | No Homology to Known |
| | | | | Proteins |
| 377 | Gma.5513.1.S1_x_at | 0.325 | AW310453 | Metabolism |
| 378 | GmaAffx.26859.1.S1_s_at | 0.325 | BM085620 | Disease & Defense |
| 379 | Gma.8490.1.S1_at | 0.323 | AB030492.1 | Metabolism |
| 380 | Gma.10342.1.S1_at | 0.323 | BE058741 | Unclassified - Hypothetical |
| | | | | Protein Supported by cDNA |
| 381 | Gma.15605.1.S1_at | 0.321 | BM143798 | Transcription |
| 382 | GmaAffx.19118.1.S1_at | 0.32 | BM523731 | Unclassified - Hypothetical |
| | | | | Protein NOT Supported by |
| | | | | cDNA |
| 383 | GmaAffx.56301.1.S1_at | 0.32 | BQ610805 | No Homology to Known |
| | | | | Proteins |
| 384 | Gma.6046.1.S1_at | 0.32 | BE022758 | Unclassified - Hypothetical |
| | | | | Protein NOT Supported by |
| | | | | cDNA |
| 385 | Gma.6442.2.S1_at | 0.32 | BM270161 | Metabolism |
| 386 | GmaAffx.92166.1.S1_s_at | 0.318 | CF806313 | Signal Transduction |
| 387 | GmaAffx.3711.1.A1_at | 0.318 | BE822116 | Unclassified - Hypothetical |
| | | | | Protein NOT Supported by |
| | | | | cDNA |
| 388 | Gma.3745.1.S1_at | 0.318 | AW156339 | Secondary Metabolism |
| 389 | Gma.17798.2.S1_at | 0.317 | CD412748 | No Homology to Known |
| | | | | Proteins |
| 390 | GmaAffx.21924.1.S1_at | 0.316 | BQ611867 | Disease & Defense |
| 391 | GmaAffx.37615.1.S1_at | 0.315 | BQ454150 | No Homology to Known |
| | | | | Proteins |
| 392 | Gma.1394.1.S1_at | 0.315 | BG839300 | Metabolism |
| 393 | GmaAffx.89772.10.A1_s_at | 0.315 | CK606097 | Disease & Defense |
| 394 | Gma.5215.1.S1_at | 0.313 | AI988141 | Unclassified - Hypothetical |
| | | | | Protein Supported by cDNA |
| 395 | Gma.6623.1.S1_at | 0.313 | BI702268 | Unclassified -Protein with |
| | | | | Unknown Function |
| 396 | GmaAffx.74573.1.S1_at | 0.311 | BU763133 | No Homology to Known |
| | | | | Proteins |
| 397 | GmaAffx.21978.1.A1_at | 0.311 | CA803009 | No Homology to Known |
| | | | | Proteins |
| 398 | GmaAffx.76346.1.A1_at | 0.311 | BQ612378 | Energy |
| 399 | Gma.1137.1.S1_x_at | 0.31 | BF067710 | Metabolism |
| 400 | GmaAffx.81362.1.S1_at | 0.309 | BF324988 | Cell Structure |
| 401 | GmaAffx.85528.1.S1_at | 0.309 | BE608070 | Unclassified - Hypothetical |
| | | | | Protein Supported by cDNA |
| 402 | GmaAffx.55124.1.S1_at | 0.309 | BI970821 | Unclassified - Hypothetical |
| | | | | Protein Supported by cDNA |
| 403 | GmaAffx.77269.1.S1_at | 0.308 | BM093698 | No Homology to Known |
| | | | | Proteins |
| 404 | Gma.15568.1.S1_at | 0.305 | AI442650 | Disease & Defense |
| 405 | Gma.17686.1.S1_at | 0.304 | BF597378 | Metabolism |
| 406 | Gma.15469.1.S1_at | 0.302 | BU546655 | Cell Structure |
| 407 | Gma.15155.2.S1_a_at | 0.302 | BG789619 | Unclassified - Hypothetical |
| | | | | Protein Supported by cDNA |
| 408 | GmaAffx.57748.1.S1_at | 0.302 | AW306274 | No Homology to Known |
| | | | | Proteins |
| 409 | Gma.3976.1.S1_at | 0.301 | BI972268 | No Homology to Known |
| | | | | Proteins |
| 410 | Gma.15155.1.S1_a_at | 0.301 | BF423817 | Unclassified - Hypothetical |
| | | | | Protein Supported by cDNA |
| 411 | GmaAffx.61130.1.S1_at | 0.3 | CA799668 | Transporter |
| 412 | GmaAffx.41078.1.S1_at | 0.3 | BI317149 | Metabolism |
| 413 | Gma.4093.2.S1_at | 0.3 | BQ081460 | Transporter |
| 414 | GmaAffx.62509.2.S1_at | 0.299 | BE021475 | Transcription |
| 415 | GmaAffx.37055.1.A1_at | 0.299 | BM142939 | Metabolism |
| 416 | GmaAffx.68724.1.A1_at | 0.299 | BI970438 | No Homology to Known |
| | | | | Proteins |
| 417 | Gma.5867.1.A1_s_at | 0.297 | BI316163 | Metabolism |
| 418 | Gma.752.1.A1_at | 0.297 | AW432463 | Transcription |
| 419 | GmaAffx.9926.1.A1_at | 0.296 | CD411291 | No Homology to Known |
| | | | | Proteins |
| 420 | Gma.4249.1.S1_at | 0.296 | BF324893 | Secondary Metabolism |
| 421 | GmaAffx.78163.1.S1_s_at | 0.296 | BM520857 | Signal Transduction |
| 422 | Gma.968.1.S1_at | 0.296 | D28876.1 | Protein Destination & Storage |
| 423 | GmaAffx.38555.2.S1_at | 0.296 | BI425873 | No Homology to Known |
| | | | | Proteins |
| 424 | Gma.7454.1.S1_a_at | 0.296 | CD405901 | Transcription |
| 425 | GmaAffx.93328.1.S1_s_at | 0.295 | CF809053 | Secondary Metabolism |
| 426 | Gma.2475.1.S1_at | 0.295 | BM522941 | Cell Structure |
| 427 | Gma.7120.1.S1_at | 0.293 | BM093481 | Metabolism |
| 428 | Gma.1716.1.S1_at | 0.292 | AW156801 | Unclassified - Hypothetical |
| | | | | Protein Supported by cDNA |
| 429 | Gma.14338.1.A1_at | 0.29 | CF923111 | Protein Destination & Storage |
| 430 | GmaAffx.4968.1.S1_at | 0.289 | BI967691 | Unclassified - Hypothetical |
| | | | | Protein Supported by cDNA |
| 431 | Gma.10559.1.S1_s_at | 0.289 | BU544359 | No Homology to Known |
| | | | | Proteins |
| 432 | GmaAffx.16986.2.S1_at | 0.287 | AW277368 | Metabolism |
| 433 | GmaAffx.24102.1.S1_at | 0.286 | BQ453911 | Unclassified - Hypothetical |
| | | | | Protein Supported by cDNA |
| 434 | GmaAffx.70005.1.S1_s_at | 0.284 | CA936335 | Secondary Metabolism |
| 435 | Gma.17921.2.S1_s_at | 0.284 | BM085020 | Metabolism |
| 436 | Gma.14471.2.S1_at | 0.284 | CD408292 | Transporter |
| 437 | Gma.5863.2.S1_s_at | 0.283 | AW350762 | Secondary Metabolism |
| 438 | GmaAffx.56715.1.S1_at | 0.283 | BG041616 | Disease & Defense |
| 439 | Gma.364.1.A1_at | 0.283 | AW309875 | Unclassified - Hypothetical |
| | | | | Protein Supported by cDNA |
| 440 | GmaAffx.90970.1.S1_s_at | 0.28 | CF806695 | Unclassified - Hypothetical |
| | | | | Protein Supported by cDNA |
| 441 | GmaAffx.13912.1.S1_at | 0.28 | BI699258 | Disease & Defense |
| 442 | Gma.5513.1.S1_at | 0.277 | AW310453 | Metabolism |
| 443 | GmaAffx.52826.3.S1_at | 0.276 | BF596503 | Signal Transduction |
| 444 | GmaAffx.71308.2.A1_at | 0.276 | AW569888 | No Homology to Known |
| | | | | Proteins |
| 445 | Gma.12684.1.S1_at | 0.275 | CD394773 | Transcription |
| 446 | GmaAffx.93220.1.S1_at | 0.275 | CF808945 | No Homology to Known |
| | | | | Proteins |
| 447 | Gma.13150.1.A1_at | 0.274 | CD411577 | Unclassified -Protein with |
| | | | | Unknown Function |
| 448 | GmaAffx.57469.1.S1_at | 0.274 | AW351054 | Unclassified - Hypothetical |
| | | | | Protein Supported by cDNA |
| 449 | Gma.2842.1.S1_at | 0.274 | BG511652 | Unclassified -Protein with |
| | | | | Unknown Function |
| 450 | GmaAffx.86745.1.S1_s_at | 0.271 | BI424960 | Disease & Defense |
| 451 | Gma.6599.1.S1_at | 0.271 | BQ629084 | Cell Structure |
| 452 | GmaAffx.75697.1.S1_s_at | 0.27 | BU090518 | Unclassified - Hypothetical |
| | | | | Protein Supported by cDNA |
| 453 | Gma.6033.1.S1_at | 0.27 | BI967767 | Unclassified - Hypothetical |
| | | | | Protein Supported by cDNA |
| 454 | Gma.4837.1.S1_at | 0.269 | BI969343 | Transporter |
| 455 | Gma.382.1.A1_at | 0.268 | AW349068 | Metabolism |
| 456 | GmaAffx.91555.1.A1_s_at | 0.268 | CF807280 | Metabolism |
| 457 | GmaAffx.9911.1.S1_at | 0.267 | BU547808 | Unclassified - Hypothetical |
| | | | | Protein NOT Supported by |
| | | | | cDNA |
| 458 | Gma.3604.1.S1_at | 0.267 | AW349604 | Cell Structure |
| 459 | Gma.17840.1.S1_at | 0.267 | BE821995 | No Homology to Known |
| | | | | Proteins |
| 460 | Gma.9086.1.S1_at | 0.263 | AW184932 | Cell Growth & Division |
| 461 | GmaAffx.71842.1.S1_at | 0.263 | BE555317 | Metabolism |
| 462 | GmaAffx.86247.1.S1_s_at | 0.262 | BG238674 | Unclassified - Hypothetical |
| | | | | Protein Supported by cDNA |
| 463 | GmaAffx.62509.1.S1_s_at | 0.261 | BE022332 | Transcription |
| 464 | GmaAffx.90104.1.S1_s_at | 0.26 | CF805829 | Transporter |
| 465 | Gma.4874.1.A1_at | 0.259 | AI938293 | No Homology to Known |
| | | | | Proteins |
| 466 | Gma.2414.1.S1_at | 0.259 | BU551192 | Protein Destination & Storage |
| 467 | Gma.3914.1.S1_at | 0.258 | BI702206 | Unclassified - Hypothetical |
| | | | | Protein Supported by cDNA |
| 468 | GmaAffx.84751.1.A1_at | 0.257 | AW349702 | Transcription |
| 469 | GmaAffx.11172.1.A1_at | 0.256 | CD404556 | No Homology to Known |
| | | | | Proteins |
| 470 | GmaAffx.93591.1.S1_s_at | 0.256 | CF809316 | Metabolism |
| 471 | Gma.6343.1.S1_at | 0.25 | AI442963 | Unclassified - Hypothetical |
| | | | | Protein NOT Supported by |
| | | | | cDNA |
| 472 | Gma.6999.2.S1_s_at | 0.249 | X60043.1 | Disease & Defense |
| 473 | Gma.4222.1.S1_at | 0.248 | BU083971 | Unclassified - Hypothetical |
| | | | | Protein NOT Supported by |
| | | | | cDNA |
| 474 | GmaAffx.51409.1.S1_at | 0.247 | BM309035 | Transporter |
| 475 | Gma.3987.1.S1_at | 0.247 | BQ453408 | Unclassified - Hypothetical |
| | | | | Protein NOT Supported by |
| | | | | cDNA |
| 476 | Gma.9828.1.S1_at | 0.245 | AF529301.1 | Metabolism |
| 477 | GmaAffx.89259.1.S1_s_at | 0.245 | CK605646 | Metabolism |
| 478 | GmaAffx.70008.1.S1_at | 0.244 | BU762337 | Transcription |
| 479 | Gma.6679.1.A1_at | 0.241 | BU544081 | Transcription |
| 480 | Gma.7422.1.S1_at | 0.24 | BQ612005 | Transporter |
| 481 | Gma.11119.2.S1_s_at | 0.24 | CF920743 | Disease & Defense |
| 482 | Gma.3744.1.S1_at | 0.239 | CD417438 | Unclassified -Protein With |
| | | | | Unknown Function |
| 483 | Gma.1137.1.S1_a_at | 0.239 | BF067710 | Metabolism |
| 484 | GmaAffx.50980.1.S1_at | 0.238 | BE823095 | Signal Transduction |
| 485 | GmaAffx.41917.1.S1_at | 0.238 | BE658926 | Disease & Defense |
| 486 | Gma.11166.1.S1_s_at | 0.237 | AW705829 | Metabolism |
| 487 | GmaAffx.71308.1.S1_at | 0.236 | BF595112 | Transcription |
| 488 | Gma.398.2.S1_at | 0.236 | BU925900 | Metabolism |
| 489 | Gma.3161.2.S1_at | 0.235 | CD400025 | Secondary Metabolism |
| 490 | GmaAffx.83818.1.S1_at | 0.235 | BU762716 | No Homology to Known |
| | | | | Proteins |
| 491 | GmaAffx.8999.2.A1_s_at | 0.234 | BI700574 | No Homology to Known |
| | | | | Proteins |
| 492 | Gma.4796.1.A1_at | 0.234 | AW102323 | Unclassified - Hypothetical |
| | | | | Protein NOT Supported by |
| | | | | cDNA |
| 493 | Gma.15093.1.S1_at | 0.234 | CD400374 | Protein Destination & Storage |
| 494 | GmaAffx.15119.1.A1_at | 0.234 | BU544528 | Metabolism |
| 495 | GmaAffx.87433.1.S1_at | 0.234 | BI701509 | Unclassified - Hypothetical |
| | | | | Protein Supported by cDNA |
| 496 | Gma.11166.1.S1_x_at | 0.233 | AW705829 | Metabolism |
| 497 | GmaAffx.74323.1.S1_at | 0.233 | BI426587 | Unclassified - Hypothetical |
| | | | | Protein NOT Supported by |
| | | | | cDNA |
| 498 | GmaAffx.50980.1.S1_x_at | 0.232 | BE823095 | Signal Transduction |
| 499 | Gma.3000.1.S1_at | 0.232 | BE657172 | No Homology to Known |
| | | | | Proteins |
| 500 | Gma.4674.1.A1_at | 0.231 | AW309836 | Metabolism |
| 501 | GmaAffx.47739.1.S1_at | 0.228 | BM522736 | Unclassified -Protein with |
| | | | | Unknown Function |
| 502 | Gma.1527.1.S1_x_at | 0.227 | BI968185 | Secondary Metabolism |
| 503 | Gma.18006.1.A1_at | 0.227 | AI496486 | Transcription |
| 504 | Gma.6999.1.S1_x_at | 0.225 | AF529303.1 | Disease & Defense |
| 505 | GmaAffx.45003.1.S1_at | 0.224 | BI968048 | Unclassified - Hypothetical |
| | | | | Protein Supported by cDNA |
| 506 | GmaAffx.91338.1.S1_at | 0.222 | CF807063 | Unclassified - Hypothetical |
| | | | | Protein Supported by cDNA |
| 507 | Gma.10559.1.S1_at | 0.221 | BU544359 | No Homology to Known |
| | | | | Proteins |
| 508 | Gma.5331.1.S1_at | 0.221 | BI892702 | Transcription |
| 509 | GmaAffx.45334.1.S1_at | 0.217 | BI969317 | Secondary Metabolism |
| 510 | GmaAffx.2026.1.A1_at | 0.216 | BU550139 | Transcription |
| 511 | Gma.3175.1.S1_at | 0.216 | AI736154 | Unclassified - Hypothetical |
| | | | | Protein NOT Supported by |
| | | | | cDNA |
| 512 | Gma.3744.2.S1_a_at | 0.215 | BI892693 | Unclassified -Protein With |
| | | | | Unknown Function |
| 513 | Gma.2252.1.S1_at | 0.215 | CD406598 | Unclassified - Hypothetical |
| | | | | Protein Supported by cDNA |
| 514 | Gma.10227.1.A1_at | 0.215 | BE020065 | Metabolism |
| 515 | GmaAffx.12933.1.S1_at | 0.211 | AW395787 | Transcription |
| 516 | GmaAffx.23292.1.A1_at | 0.211 | BI967795 | No Homology to Known |
| | | | | Proteins |
| 517 | Gma.7549.1.S1_at | 0.21 | BG508637 | Unclassified -Protein with |
| | | | | Unknown Function |
| 518 | Gma.13218.1.A1_s_at | 0.209 | CD391892 | No Homology to Known |
| | | | | Proteins |
| 519 | GmaAffx.89772.14.A1_s_at | 0.209 | CK606614 | Disease & Defense |
| 520 | Gma.6921.1.S1_at | 0.208 | BE823209 | Signal Transduction |
| 521 | Gma.5611.1.S1_at | 0.208 | AW309826 | Metabolism |
| 522 | GmaAffx.5264.1.S1_s_at | 0.207 | BM521512 | Disease & Defense |
| 523 | GmaAffx.24521.1.S1_at | 0.207 | BU761767 | Unclassified - Hypothetical |
| | | | | Protein NOT Supported by |
| | | | | cDNA |
| 524 | GmaAffx.10582.1.S1_at | 0.205 | BM523687 | Transcription |
| 525 | Gma.6033.2.S1_at | 0.204 | AI416728 | Unclassified - Hypothetical |
| | | | | Protein Supported by cDNA |
| 526 | GmaAffx.14030.1.S1_at | 0.204 | BE021503 | No Homology to Known |
| | | | | Proteins |
| 527 | Gma.18052.1.S1_at | 0.204 | BF595464 | Transporter |
| 528 | GmaAffx.59470.1.S1_at | 0.202 | BI469872 | Secondary Metabolism |
| 529 | Gma.3165.1.S1_at | 0.202 | CD406755 | Transcription |
| 530 | Gma.1326.1.S1_at | 0.198 | CD397515 | Secondary Metabolism |
| 531 | Gma.6192.1.S1_at | 0.197 | AW396773 | Metabolism |
| 532 | GmaAffx.61593.1.S1_at | 0.197 | BU765478 | Unclassified - Hypothetical |
| | | | | Protein NOT Supported by |
| | | | | cDNA |
| 533 | Gma.9638.1.A1_at | 0.195 | CA936403 | Disease & Defense |
| 534 | GmaAffx.71222.1.S1_at | 0.194 | AW460050 | Metabolism |
| 535 | GmaAffx.81618.1.S1_at | 0.194 | AW706403 | No Homology to Known |
| | | | | Proteins |
| 536 | Gma.3591.2.S1_at | 0.193 | BE473658 | Secondary Metabolism |
| 537 | Gma.1269.1.S1_at | 0.192 | AW309671 | Metabolism |
| 538 | Gma.857.1.S1_at | 0.192 | BE822264 | No Homology to Known |
| | | | | Proteins |
| 539 | Gma.6643.1.A1_at | 0.191 | BE823205 | Secondary Metabolism |
| 540 | Gma.8370.1.S1_at | 0.191 | BQ454087 | Signal Transduction |
| 541 | GmaAffx.57967.1.S1_at | 0.19 | AW102170 | Protein Destination & Storage |
| 542 | Gma.5469.1.S1_at | 0.19 | AW349200 | No Homology to Known |
| | | | | Proteins |
| 543 | GmaAffx.70435.2.S1_at | 0.189 | AW705853 | Transcription |
| 544 | Gma.6081.1.S1_at | 0.187 | BI967474 | Disease & Defense |
| 545 | Gma.1394.2.S1_at | 0.186 | BI470370 | Metabolism |
| 546 | Gma.17877.1.S1_at | 0.185 | BQ612471 | Unclassified -Protein with |
| | | | | Unknown Function |
| 547 | Gma.15996.1.S1_x_at | 0.184 | BQ454193 | Unclassified - Hypothetical |
| | | | | Protein Supported by cDNA |
| 548 | Gma.6075.1.S1_at | 0.184 | AW317183 | Disease & Defense |
| 549 | Gma.16947.1.S1_at | 0.184 | BG155375 | Unclassified - Hypothetical |
| | | | | Protein Supported by cDNA |
| 550 | Gma.7788.1.A1_at | 0.182 | BU549148 | Unclassified - Hypothetical |
| | | | | Protein Supported by cDNA |
| 551 | GmaAffx.16986.1.A1_s_at | 0.182 | AW351116 | Metabolism |
| 552 | Gma.8489.1.S1_s_at | 0.181 | AB030493.1 | Metabolism |
| 553 | Gma.1527.1.S1_a_at | 0.18 | BI968185 | Secondary Metabolism |
| 554 | GmaAffx.90395.1.A1_s_at | 0.177 | CF807499 | Unclassified -Protein with |
| | | | | Unknown Function |
| 555 | Gma.6643.2.S1_at | 0.177 | BE023121 | Secondary Metabolism |
| 556 | Gma.6999.1.S1_s_at | 0.174 | AF529303.1 | Disease & Defense |
| 557 | GmaAffx.5531.1.S1_at | 0.167 | BQ740711 | Cell Structure |
| 558 | Gma.15534.1.S1_at | 0.162 | U50081.1 | Metabolism |
| 559 | Gma.3578.1.S1_at | 0.162 | AW156522 | Disease & Defense |
| 560 | Gma.6999.3.S1_s_at | 0.162 | CF921432 | Disease & Defense |
| 561 | GmaAffx.59808.1.S1_at | 0.161 | BM886228 | Transporter |
| 562 | GmaAffx.11897.1.S1_at | 0.16 | BE819868 | Transporter |
| 563 | GmaAffx.295.1.A1_s_at | 0.159 | BG653267 | Protein Destination & Storage |
| 564 | Gma.5309.1.S1_at | 0.158 | BM092263 | Unclassified - Hypothetical |
| | | | | Protein NOT Supported by |
| | | | | cDNA |
| 565 | GmaAffx.70959.1.S1_at | 0.157 | BE611219 | Unclassified - Hypothetical |
| | | | | Protein NOT Supported by |
| | | | | cDNA |
| 566 | GmaAffx.91428.1.A1_at | 0.157 | CF807137 | Metabolism |
| 567 | GmaAffx.19467.1.S1_at | 0.155 | BQ453237 | Signal Transduction |
| 568 | GmaAffx.40923.1.S1_at | 0.154 | BG653848 | Disease & Defense |
| 569 | Gma.8187.1.S1_a_at | 0.154 | BU549133 | Signal Transduction |
| 570 | Gma.9010.1.S1_at | 0.153 | CF921194 | Disease & Defense |
| 571 | GmaAffx.32606.1.S1_at | 0.153 | BM270707 | Unclassified - Hypothetical |
| | | | | Protein NOT Supported by |
| | | | | cDNA |
| 572 | Gma.11731.1.A1_at | 0.15 | CD392320 | Unclassified -Protein with |
| | | | | Unknown Function |
| 573 | Gma.1758.1.S1_at | 0.15 | CD402046 | Unclassified -Protein with |
| | | | | Unknown Function |
| 574 | Gma.11028.2.S1_s_at | 0.149 | CA782711 | Metabolism |
| 575 | Gma.4873.1.S1_at | 0.148 | BI969532 | Signal Transduction |
| 576 | Gma.15036.2.A1_at | 0.148 | CD411972 | Unclassified -Protein with |
| | | | | Unknown Function |
| 577 | Gma.17662.2.S1_s_at | 0.146 | BQ630190 | Unclassified - Hypothetical |
| | | | | Protein NOT Supported by |
| | | | | cDNA |
| 578 | GmaAffx.9944.1.S1_at | 0.146 | BM567882 | Cell Structure |
| 579 | Gma.10216.1.S1_at | 0.145 | BI969369 | Metabolism |
| 580 | Gma.3578.1.S1_s_at | 0.142 | AW156522 | Disease & Defense |
| 581 | GmaAffx.51464.1.S1_at | 0.142 | BQ628397 | No Homology to Known |
| | | | | Proteins |
| 582 | Gma.336.1.A1_at | 0.142 | AW310815 | Metabolism |
| 583 | Gma.7580.1.S1_at | 0.139 | BE057763 | Unclassified - Hypothetical |
| | | | | Protein Supported by cDNA |
| 584 | Gma.1445.1.S1_at | 0.138 | BQ454241 | Metabolism |
| 585 | Gma.12227.1.S1_at | 0.136 | BG511671 | Unclassified - Hypothetical |
| | | | | Protein NOT Supported by |
| | | | | cDNA |
| 586 | GmaAffx.68507.1.S1_at | 0.136 | BI699405 | Transcription |
| 587 | GmaAffx.41230.1.A1_at | 0.135 | BI471609 | Unclassified - Hypothetical |
| | | | | Protein NOT Supported by |
| | | | | cDNA |
| 588 | Gma.4260.1.S1_at | 0.134 | BM177256 | Unclassified - Hypothetical |
| | | | | Protein NOT Supported by |
| | | | | cDNA |
| 589 | Gma.320.1.S1_at | 0.13 | AF019116.1 | Disease & Defense |
| 590 | Gma.3810.1.S1_s_at | 0.125 | BI972063 | Disease & Defense |
| 591 | Gma.6895.1.S1_a_at | 0.122 | BU545788 | Metabolism |
| 592 | GmaAffx.12665.1.A1_at | 0.121 | BE822073 | Metabolism |
| 593 | Gma.13062.1.S1_at | 0.12 | CD394315 | Metabolism |
| 594 | Gma.6462.2.S1_a_at | 0.119 | BE555263 | Unclassified - Hypothetical |
| | | | | Protein NOT Supported by |
| | | | | cDNA |
| 595 | GmaAffx.65393.1.S1_s_at | 0.119 | BU760770 | No Homology to Known |
| | | | | Proteins |
| 596 | Gma.3774.2.S1_at | 0.118 | CD393365 | Energy |
| 597 | Gma.2253.1.S1_at | 0.114 | AF169018.1 | Metabolism |
| 598 | GmaAffx.92479.1.S1_s_at | 0.112 | CF808204 | No Homology to Known |
| | | | | Proteins |
| 599 | Gma.17446.1.A1_at | 0.106 | AI461103 | Unclassified -Protein with |
| | | | | Unknown Function |
| 600 | Gma.13854.1.S1_at | 0.106 | CD408166 | Metabolism |
| 601 | Gma.15.1.S1_at | 0.106 | U66316.1 | Unclassified -Protein with |
| | | | | Unknown Function |
| 602 | GmaAffx.87417.1.S1_at | 0.104 | BM568072 | Metabolism |
| 603 | GmaAffx.52963.1.S1_at | 0.104 | AW310673 | Unclassified -Protein with |
| | | | | Unknown Function |
| 604 | GmaAffx.26908.1.S1_at | 0.101 | BF425834 | Secondary Metabolism |
| 605 | Gma.17890.1.S1_at | 0.101 | AW432965 | No Homology to Known |
| | | | | Proteins |
| 606 | GmaAffx.81750.1.S1_at | 0.0985 | AW306336 | No Homology to Known |
| | | | | Proteins |
| 607 | Gma.12393.3.S1_at | 0.098 | BM085488 | Secondary Metabolism |
| 608 | GmaAffx.89407.1.A1_s_at | 0.0978 | CK606273 | Transporter |
| 609 | GmaAffx.17961.1.A1_at | 0.0974 | BE657601 | Protein Destination & Storage |
| 610 | Gma.3519.1.S1_at | 0.0892 | CD403581 | Unclassified - Hypothetical |
| | | | | Protein Supported by cDNA |
| 611 | Gma.9740.1.S1_at | 0.0859 | BQ081058 | Unclassified - Hypothetical |
| | | | | Protein Supported by cDNA |
| 612 | Gma.16566.1.S1_at | 0.0856 | CD400974 | Metabolism |
| 613 | Gma.2133.1.S1_at | 0.0855 | AW310071 | Unclassified -Protein with |
| | | | | Unknown Function |
| 614 | Gma.1243.1.S1_at | 0.0845 | AW311027 | Unclassified - Hypothetical |
| | | | | Protein Supported by cDNA |
| 615 | Gma.15625.1.S1_x_at | 0.0841 | U77678.1 | Metabolism |
| 616 | Gma.3000.2.S1_a_at | 0.0841 | BM270844 | No Homology to Known |
| | | | | Proteins |
| 617 | GmaAffx.90395.1.S1_s_at | 0.0837 | CF806120 | Unclassified -Protein with |
| | | | | Unknown Function |
| 618 | Gma.2986.1.S1_at | 0.0827 | BM271324 | Metabolism |
| 619 | Gma.6192.1.S1_s_at | 0.0814 | AW396773 | Metabolism |
| 620 | GmaAffx.90600.1.S1_at | 0.0806 | CF806325 | Metabolism |
| 621 | Gma.15625.1.S1_a_at | 0.0771 | U77678.1 | Metabolism |
| 622 | Gma.4687.1.S1_at | 0.0751 | AW309716 | Secondary Metabolism |
| 623 | Gma.11211.1.S1_s_at | 0.074 | AI442578 | No Homology to Known |
| | | | | Proteins |
| 624 | GmaAffx.24413.1.A1_at | 0.0704 | BU548368 | Metabolism |
| 625 | Gma.8365.1.S1_at | 0.07 | CD396813 | Unclassified - Hypothetical |
| | | | | Protein NOT Supported by |
| | | | | cDNA |
| 626 | Gma.2912.1.S1_s_at | 0.0691 | AF117884.1 | Protein Destination & Storage |
| 627 | Gma.17985.1.S1_at | 0.0678 | CD416450 | Metabolism |
| 628 | GmaAffx.73698.1.S1_at | 0.0673 | BU964780 | Metabolism |
| 629 | GmaAffx.90600.1.S1_s_at | 0.065 | CF806325 | Metabolism |
| 630 | GmaAffx.6635.1.S1_at | 0.0639 | CD412718 | Disease & Defense |
| 631 | Gma.17516.1.S1_at | 0.0631 | CD396417 | Transporter |
| 632 | Gma.1527.2.S1_at | 0.0624 | BE658017 | Secondary Metabolism |
| 633 | Gma.176.1.S1_s_at | 0.0618 | AF004810.1 | Protein Destination & Storage |
| 634 | Gma.1332.1.S1_x_at | 0.0609 | BI970666 | Secondary Metabolism |
| 635 | Gma.9947.1.S1_at | 0.0589 | BF324269 | Unclassified - Hypothetical |
| | | | | Protein Supported by cDNA |
| 636 | Gma.8425.1.S1_s_at | 0.0562 | D83968.1 | Metabolism |
| 637 | Gma.8318.1.A1_at | 0.0544 | BU550469 | No Homology to Known |
| | | | | Proteins |
| 638 | Gma.2912.1.S1_at | 0.0517 | AF117884.1 | Protein Destination & Storage |
| 639 | Gma.79.1.S1_x_at | 0.0493 | S44202.1 | Unclassified -Protein with |
| | | | | Unknown Function |
| 640 | Gma.14471.1.S1_s_at | 0.0479 | CF921241 | No Homology to Known |
| | | | | Proteins |
| 641 | Gma.1314.1.S1_at | 0.0462 | U63726.1 | Metabolism |
| 642 | GmaAffx.19541.1.S1_s_at | 0.044 | BQ612990 | No Homology to Known |
| | | | | Proteins |
| 643 | GmaAffx.10204.1.S1_at | 0.0421 | BQ298213 | Unclassified - Hypothetical |
| | | | | Protein Supported by cDNA |
| 644 | Gma.5947.1.S1_s_at | 0.0413 | BQ610509 | Disease & Defense |
| 645 | Gma.3774.1.S1_at | 0.0383 | BU550698 | Energy |
| 646 | Gma.79.1.S1_s_at | 0.0381 | S44202.1 | Unclassified -Protein with |
| | | | | Unknown Function |
| 647 | GmaAffx.68621.1.A1_at | 0.0301 | BI893889 | Unclassified -Protein with |
| | | | | Unknown Function |
| 648 | GmaAffx.85382.1.S1_s_at | 0.0234 | BI945154 | No Homology to Known |
| | | | | Proteins |
| 649 | Gma.3774.2.S1_a_at | 0.0216 | CD393365 | Energy |
| 650 | Gma.15307.1.S1_at | 0.0127 | U64866.1 | Disease & Defense |

| ID | Sub-Family | Gene Family | Additional Description |
|---|---|---|---|
| 1 | | | Predicted carbonic anhydrase involved in |
| | | | protection against oxidative damage |
| 2 | | | |
| 3 | | | |
| 4 | Inorganic | Fe(II) | Fe2+/Zn2+ regulated transporter |
| | Solute | transporter | |
| | Cotransporters | isolog family | |
| 5 | | | Gelatinase A and related matrix |
| | | | metalloproteases |
| 6 | | | |
| 7 | | | |
| 8 | | | Tyrosine aminotransferase |
| 9 | | | Predicted transporter (major facilitator |
| | | | superfamily) |
| 10 | | | |
| 11 | | | Cytochrome b5 |
| 11 | | | NADH-cytochrome b-5 reductase |
| 11 | | | Sulfite oxidase, molybdopterin-binding |
| | | | component |
| 12 | Protein | | |
| | Kinase | | |
| 13 | | | |
| 14 | | | Glutathione S-transferase |
| 15 | | | Cytochrome b5 |
| 15 | | | NADH-cytochrome b-5 reductase |
| 15 | | | Sulfite oxidase, molybdopterin-binding |
| | | | component |
| 16 | | | |
| 17 | | | |
| 18 | | | |
| 19 | | | Thioredoxin |
| 20 | | | |
| 21 | | | |
| 22 | | | Ca2+-dependent lipid-binding protein |
| | | | CLB1/vesicle protein vp115/Granuphilin A, |
| | | | contains C2 domain |
| 23 | Protein | | |
| | Kinase | | |
| 24 | | | Sulfate/bicarbonate/oxalate exchanger SAT- |
| | | | 1 and related transporters (SLC26 family) |
| 25 | | | Tyrosine aminotransferase |
| 26 | | | Gamma-glutamyltransferase |
| 27 | | | |
| 28 | Storage | | |
| | Protein | | |
| 29 | | | |
| 30 | | | |
| 31 | | | RNA-binding protein Sam68 and related |
| | | | KH domain proteins |
| 32 | | | UDP-glucuronosyl and UDP-glucosyl |
| | | | transferase |
| 33 | | | |
| 34 | | | Uncharacterized conserved protein |
| 35 | | | |
| 36 | | | |
| 37 | | | |
| 38 | Transcription | Basic Leucine | |
| | Factor | Zipper (bZIP) | |
| 39 | | | |
| 40 | | | |
| 41 | | | |
| 42 | Cytochrome | CYP76C | Cytochrome P450 CYP2 subfamily |
| | P450 | | |
| 43 | | | |
| 44 | | | |
| 45 | Cytochrome | CYP82C | Cytochrome P450 CYP2 subfamily |
| | P450 | | |
| 46 | | | |
| 47 | | | |
| 48 | | | |
| 49 | | | |
| 50 | | | |
| 51 | | | |
| 52 | Protein | | Glycerophosphoryl diester |
| | Kinase | | phosphodiesterase |
| 52 | Protein | | Serine/threonine protein kinase |
| | Kinase | | |
| 53 | | | |
| 54 | | | |
| 55 | Transcription | Basic Helix- | |
| | Factor | Loop-Helix | |
| | | (bHLH) | |
| 56 | | | Predicted membrane protein, contains DoH |
| | | | and Cytochrome b-561/ferric reductase |
| | | | transmembrane domains |
| 57 | | | Copper chaperone |
| 58 | | | |
| 59 | | | |
| 60 | | | Glutaredoxin and related proteins |
| 61 | | | |
| 62 | Transcription | AP2/EREBP | Uncharacterized conserved protein |
| | Factor | | |
| 63 | | | |
| 64 | Unclassified | | FOG: Leucine rich repeat |
| 65 | | | |
| 66 | | | |
| 67 | | | Apolipoprotein D/Lipocalin |
| 68 | | | |
| 69 | | | |
| 70 | | | Glutathione S-transferase |
| 71 | | | |
| 72 | Transcription | NAC Domain | |
| | Factor | | |
| 73 | | | Carbon-nitrogen hydrolase |
| 74 | | | Fasciclin and related adhesion glycoproteins |
| 75 | | | Aldehyde dehydrogenase |
| 76 | | | |
| 77 | | | |
| 78 | | | Translation initiation factor 5A (eIF-5A) |
| 79 | | | |
| 80 | | | Inositol polyphosphate 5-phosphatase and |
| | | | related proteins |
| 81 | | | Annexin |
| 82 | | | |
| 83 | | | |
| 84 | | | |
| 85 | | | |
| 86 | | | |
| 87 | | | Hydroxyindole-O-methyltransferase and |
| | | | related SAM-dependent methyltransferases |
| 88 | | | Endoplasmic reticulum protein EP58, |
| | | | contains filamin rod domain and KDEL |
| | | | motif |
| 89 | | | |
| 90 | Ethylene | | Iron/ascorbate family oxidoreductases |
| | Biosynthesis | | |
| 91 | | | |
| 92 | | | Ca2+-independent phospholipase A2 |
| 93 | | | Amino acid transporters |
| 94 | | | Molecular chaperones HSP70/HSC70, |
| | | | HSP70 superfamily |
| 95 | | | Predicted carbonic anhydrase involved in |
| | | | protection against oxidative damage |
| 96 | | | |
| 97 | | | |
| 98 | | | |
| 99 | | | |
| 100 | | | |
| 101 | | | |
| 102 | Transcription | AP2/EREBP | |
| | Factor | | |
| 103 | | | |
| 104 | | | Alpha-L-fucosidase |
| 105 | | | Iron/ascorbate family oxidoreductases |
| 106 | Protein | | Serine/threonine protein kinase |
| | Kinase | | |
| 107 | | | Serine carboxypeptidases (lysosomal |
| | | | cathepsin A) |
| 108 | | | Beta-glucosidase, lactase |
| | | | phlorizinhydrolase, and related proteins |
| 109 | | | |
| 110 | | | Serine carboxypeptidases |
| 111 | Protein | | |
| | Kinase | | |
| 112 | | | |
| 113 | | | |
| 114 | | | |
| 115 | | | |
| 116 | | | Magnesium transporters: CorA family |
| 117 | | | Acyl-CoA synthetase |
| 118 | | | |
| 119 | | | |
| 120 | | | |
| 121 | Protein | | Mannosyltransferase |
| | Kinase | | |
| 122 | | | |
| 123 | Ethylene | | 1-aminocyclopropane-1-carboxylate |
| | Biosynthesis | | synthase, and related proteins |
| 124 | | | |
| 125 | | | Multidrug/pheromone exporter, ABC |
| | | | superfamily |
| 126 | | | Kynurenine 3-monooxygenase and related |
| | | | flavoprotein monooxygenases |
| 127 | | | |
| 128 | | | Annexin |
| 129 | | | |
| 130 | | | |
| 131 | Protein | AtCIPKs | Serine/threonine protein kinase |
| | Kinase | | |
| 132 | | | |
| 133 | | | |
| 134 | Transcription | Basic Helix- | |
| | Factor | Loop-Helix | |
| | | (bHLH) | |
| 135 | Calcium- | | |
| | Binding | | |
| 136 | | | Predicted haloacid dehalogenase-like |
| | | | hydrolase |
| 137 | | | Predicted E3 ubiquitin ligase |
| 138 | Protein | | |
| | Kinase | | |
| 139 | | | Ubiquitin-protein ligase |
| 140 | | | Leucine rich repeat proteins, some proteins |
| | | | contain F-box |
| 141 | | | |
| 142 | | | |
| 143 | | | Transporter, ABC superfamily (Breast |
| | | | cancer resistance protein) |
| 144 | | | |
| 145 | Ethylene | | Iron/ascorbate family oxidoreductases |
| | Biosynthesis | | |
| 146 | | | |
| 147 | Transcription | Basic Helix- | |
| | Factor | Loop-Helix | |
| | | (bHLH) | |
| 148 | Transcription | Homeodomain | Transcription factor HEX, contains HOX |
| | Factor | | and HALZ domains |
| 149 | Transcription | AUX/IAA | |
| | Factor | | |
| 150 | | | Endoplasmic reticulum protein EP58, |
| | | | contains filamin rod domain and KDEL |
| | | | motif |
| 151 | Ethylene | | Iron/ascorbate family oxidoreductases |
| | Biosynthesis | | |
| 152 | | | |
| 153 | | | |
| 154 | | | |
| 155 | | | Multicopper oxidases |
| 156 | Protein | | Serine/threonine protein kinase |
| | Kinase | | |
| 157 | | | |
| 158 | | | |
| 159 | | | FOG: PPR repeat |
| 160 | Histidine | | Sensory transduction histidine kinase |
| | Kinase | | |
| 161 | | | Chitinase |
| 162 | Transcription | Homeodomain | Transcription factor MEIS1 and related |
| | Factor | | HOX domain proteins |
| 163 | | | |
| 164 | | | Zn-finger protein |
| 165 | | | Ca2+/calmodulin-dependent protein kinase, |
| | | | EF-Hand protein superfamily |
| 166 | | | Speckle-type POZ protein SPOP and related |
| | | | proteins with TRAF, MATH and BTB/POZ |
| | | | domains |
| 167 | | | |
| 168 | | | |
| 169 | | | Transporter, ABC superfamily (Breast |
| | | | cancer resistance protein) |
| 170 | | | |
| 171 | | | |
| 172 | | | |
| 173 | | | |
| 174 | | | |
| 175 | | | |
| 176 | Transcription | ARF | |
| | Factor | | |
| 177 | Ethylene | | Iron/ascorbate family oxidoreductases |
| | Biosynthesis | | |
| 178 | | | |
| 179 | | | Glyoxylate/hydroxypyruvate reductase (D- |
| | | | isomer-specific 2-hydroxy acid |
| | | | dehydrogenase superfamily) |
| 180 | | | Hydroxyindole-O-methyltransferase and |
| | | | related SAM-dependent methyltransferases |
| 181 | | | |
| 182 | Transcription | NAC Domain | |
| | Factor | | |
| 183 | Protein | | Serine/threonine protein kinase |
| | Kinase | | |
| 184 | | | Kelch repeat-containing proteins |
| 185 | | | Aquaporin (major intrinsic protein family) |
| 186 | | | |
| 187 | | | ER lumen protein retaining receptor |
| 188 | | | |
| 189 | | | |
| 190 | | | |
| 191 | Transcription | Homeodomain | Transcription factor HEX, contains HOX |
| | Factor | | and HALZ domains |
| 192 | | | FOG: Predicted E3 ubiquitin ligase |
| 193 | | | |
| 194 | | | 17 beta-hydroxysteroid dehydrogenase type |
| | | | 3, HSD17B3 |
| 195 | Protein | | Glycogen synthase kinase-3 |
| | Kinase | | |
| 196 | | | |
| 197 | Transcription | Basic Helix- | |
| | Factor | Loop-Helix | |
| | | (bHLH) | |
| 198 | | | |
| 199 | | | Dehydrogenase kinase |
| 200 | | | O-methyltransferase |
| 201 | | | |
| 202 | | | |
| 203 | Trehalose | | Trehalose-6-phosphate synthase component |
| | Biosynthesis | | TPS1 and related subunits |
| 204 | | | Homocysteine S-methyltransferase |
| 205 | | | |
| 206 | | | |
| 207 | | | |
| 208 | | | |
| 209 | | | |
| 210 | | | |
| 211 | | | |
| 212 | | | |
| 213 | Transcription | Basic Leucine | |
| | Factor | Zipper (bZIP) | |
| 214 | | | Glutathione S-transferase |
| 215 | | | Leucine rich repeat proteins, some proteins |
| | | | contain F-box |
| 216 | | | |
| 217 | | | |
| 218 | | | Predicted transporter (major facilitator |
| | | | superfamily) |
| 219 | Transcription | NAC Domain | |
| | Factor | | |
| 220 | Protein | | Serine/threonine protein phosphatase |
| | Phosphatase | | |
| 221 | | | Amino acid transporters |
| 222 | | | |
| 223 | | | |
| 224 | | | |
| 225 | | | |
| 226 | | | Hydroxyindole-O-methyltransferase and |
| | | | related SAM-dependent methyltransferases |
| 227 | | | Serine carboxypeptidases (lysosomal |
| | | | cathepsin A) |
| 228 | | | |
| 229 | | | Ca2+-binding protein (centrin/caltractin). |
| | | | EF-Hand superfamily protein |
| 230 | Transcription | Unclassified | CCAAT-binding factor, subunit B (HAP2) |
| | Factor | | |
| 231 | | | Arylacetamide deacetylase |
| 232 | | | |
| 233 | | | |
| 234 | | | |
| 235 | | | Phosphatidylethanolamine binding protein |
| 236 | | | Permease of the major facilitator |
| | | | superfamily |
| 237 | Transcription | Unclassified | |
| | Factor | | |
| 238 | | | Aquaporin (major intrinsic protein family) |
| 239 | | | |
| 240 | | | |
| 241 | | | |
| 242 | | | |
| 243 | | | |
| 244 | | | |
| 245 | Transcription | Homeodomain | Transcription factor MEIS1 and related |
| | Factor | | HOX domain proteins |
| 246 | | | |
| 247 | | | |
| 248 | | | Alcohol dehydrogenase, class V |
| 249 | | | |
| 250 | | | |
| 251 | Cytochrome | CYP82F | Cytochrome P450 CYP2 subfamily |
| | P450 | | |
| 252 | Protein | AtCIPKs | Serine/threonine protein kinase |
| | Kinase | | |
| 253 | | | |
| 254 | | | Zn-finger protein |
| 255 | | | |
| 256 | | | |
| 257 | Transcription | AP2/EREBP | |
| | Factor | | |
| 258 | | | |
| 259 | | | Glutaredoxin and related proteins |
| 260 | | | |
| 261 | | | |
| 262 | | | |
| 263 | | | |
| 264 | | | Dipeptidyl aminopeptidase |
| 265 | | | Iron/ascorbate family oxidoreductases |
| 266 | Transcription | Basic Leucine | FOG: Armadillo/beta-catenin-like repeats |
| | Factor | Zipper (bZIP) | |
| 267 | | | |
| 268 | Cytochrome | CYP71B | Cytochrome P450 CYP2 subfamily |
| | P450 | | |
| 269 | Transcription | Basic Leucine | FOG: Armadillo/beta-catenin-like repeats |
| | Factor | Zipper (bZIP) | |
| 270 | | | UDP-glucuronosyl and UDP-glucosyl |
| | | | transferase |
| 271 | | | Microtubule-binding protein (translationally |
| | | | controlled tumor protein) |
| 272 | | | |
| 273 | | | Purple (tartrate-resistant) acid phosphatase |
| 274 | | | Beta-glucosidase, lactase |
| | | | phlorizinhydrolase, and related proteins |
| 275 | | | FOG: Predicted E3 ubiquitin ligase |
| 276 | Storage | | |
| | Protein | | |
| 277 | | | |
| 278 | | | |
| 279 | Cytochrome | CYP90C | Cytochrome P450 CYP4/CYP19/CYP26 |
| | P450 | | subfamilies |
| 280 | | | FOG: Kelch repeat |
| 281 | | | Multicopper oxidases |
| 282 | | | Predicted E3 ubiquitin ligase |
| 283 | Protein | | Serine/threonine protein kinase |
| | Kinase | | |
| 284 | | | Aldo/keto reductase family proteins |
| 285 | | | |
| 286 | | | O-methyltransferase |
| 287 | Transcription | Homeodomain | Transcription factor MEIS1 and related |
| | Factor | | HOX domain proteins |
| 288 | | | |
| 289 | | | |
| 290 | | | |
| 291 | | | |
| 292 | Transcription | Basic Helix- | |
| | Factor | Loop-Helix | |
| | | (bHLH) | |
| 293 | Protein | | Serine/threonine protein phosphatase |
| | Phosphatase | | |
| 294 | | | Annexin |
| 295 | Protein | | Serine/threonine protein phosphatase |
| | Phosphatase | | |
| 296 | | | O-methyltransferase |
| 297 | | | |
| 298 | | | Hydroxyindole-O-methyltransferase and |
| | | | related SAM-dependent methyltransferases |
| 299 | | | Chitinase |
| 300 | | | |
| 301 | | | |
| 302 | | | |
| 303 | Protein | | |
| | Kinase | | |
| 304 | | | |
| 305 | | | Arylacetamide deacetylase |
| 306 | | | Copper chaperone |
| 307 | | | |
| 308 | | | |
| 309 | | | |
| 310 | | | |
| 311 | | | Annexin |
| 312 | | | |
| 313 | | | GPI-alpha-mannosyltransferase III |
| | | | (GPII0/PIG-B) involved in |
| | | | glycosylphosphatidylinositol anchor |
| | | | biosynthesis |
| 314 | | | Predicted flavoprotein |
| 315 | | | Glyoxylate/hydroxypyruvate reductase (D- |
| | | | isomer-specific 2-hydroxy acid |
| | | | dehydrogenase superfamily) |
| 316 | | | Glutathione peroxidase |
| 317 | | | |
| 318 | | | |
| 319 | | | |
| 320 | | | Multicopper oxidases |
| 321 | | | Molybdenum cofactor sulfurase |
| 322 | | | Phenylalanine and histidine ammonia-lyase |
| 323 | | | |
| 324 | | | |
| 325 | | | |
| 326 | | | |
| 327 | | | Carbon-nitrogen hydrolase |
| 328 | Protein | | |
| | Phosphatase | | |
| 329 | | | Glutathione S-transferase |
| 330 | Amines | | Amine oxidase |
| | Biosynthesis | | |
| 331 | | | Steroid reductase |
| 332 | | | |
| 333 | Amino Acid | | Arginase family protein |
| | Biosynthesis/ | | |
| | Metabolism | | |
| 334 | | | |
| 335 | Transcription | Basic Helix- | |
| | Factor | Loop-Helix | |
| | | (bHLH) | |
| 336 | | | Aquaporin (major intrinsic protein family) |
| 337 | | | Branched chain alpha-keto acid |
| | | | dehydrogenase complex, alpha subunit |
| 338 | | | |
| 339 | | | Soluble epoxide hydrolase |
| 340 | Transcription | MYB | Transcription factor, Myb superfamily |
| | Factor | | |
| 341 | | | |
| 342 | Transcription | Homeodomain | Transcription factor HEX, contains HOX |
| | Factor | | and HALZ domains |
| 343 | | | |
| 344 | | | |
| 345 | | | Alanine-glyoxylate aminotransferase AGT2 |
| 346 | Transcription | Homeodomain | Transcription factor MEIS1 and related |
| | Factor | | HOX domain proteins |
| 347 | | | Iron/ascorbate family oxidoreductases |
| 348 | | | Aldehyde reductase |
| 349 | | | Iron/ascorbate family oxidoreductases |
| 350 | | | |
| 351 | | | |
| 352 | | | Steroid reductase |
| 353 | | | |
| 354 | Transcription | Homeodomain | Transcription factor MEIS1 and related |
| | Factor | | HOX domain proteins |
| 355 | | | |
| 356 | | | |
| 357 | | | |
| 358 | | | |
| 359 | | | |
| 360 | | | |
| 361 | | | 1,4-benzoquinone reductase-like; Trp |
| | | | repressor binding protein-like/protoplast- |
| | | | secreted protein |
| 362 | | | |
| 363 | | | |
| 364 | | | |
| 365 | | | |
| 366 | | | |
| 367 | Transcription | Homeodomain | Transcription factor HEX, contains HOX |
| | Factor | | and HALZ domains |
| 368 | Storage | | Ca2+-independent phospholipase A2 |
| | Protein | | |
| 369 | | | |
| 370 | Protein | | FOG: Transposon-encoded proteins with |
| | Kinase | | TYA, reverse transcriptase, integrase |
| | | | domains in various combinations |
| 371 | Protein | | |
| | Kinase | | |
| 372 | Transcription | NAC Domain | |
| | Factor | | |
| 373 | | | Tomosyn and related SNARE-interacting |
| | | | proteins |
| 374 | | | Pectin acetylesterase and similar proteins |
| 375 | | | Amino acid transporters |
| 376 | | | |
| 377 | | | Haloacid dehalogenase-like hydrolase |
| 378 | | | Glutathione S-transferase |
| 379 | | | Protein involved in thiamine biosynthesis |
| | | | and DNA damage tolerance |
| 380 | | | |
| 381 | Transcription | AP2/EREBP | |
| | Factor | | |
| 382 | | | |
| 383 | | | |
| 384 | | | |
| 385 | | | Lysophospholipase |
| 386 | Unclassified | | Aspartyl protease |
| 387 | | | |
| 388 | Amines | | Amine oxidase |
| | Biosynthesis | | |
| 389 | | | |
| 390 | | | |
| 391 | | | |
| 392 | | | |
| 393 | | | |
| 394 | | | BCL2-associated athanogene-like proteins |
| | | | and related BAG family chaperone |
| | | | regulators |
| 395 | | | 1,4-benzoquinone reductase-like; Trp |
| | | | repressor binding protein-like/protoplast- |
| | | | secreted protein |
| 396 | | | |
| 397 | | | |
| 398 | | | 1,4-benzoquinone reductase-like; Trp |
| | | | repressor binding protein-like/protoplast- |
| | | | secreted protein |
| 399 | | | |
| 400 | | | |
| 401 | | | FOG: PPR repeat |
| 402 | | | Metacaspase involved in regulation of |
| | | | apoptosis |
| 403 | | | |
| 404 | | | |
| 405 | Cytochrome | CYP76C | Cytochrome P450 CYP2 subfamily |
| | P450 | | |
| 406 | | | Alpha tubulin |
| 407 | | | |
| 408 | | | |
| 409 | | | |
| 410 | | | |
| 411 | | | H+/oligopeptide symporter |
| 412 | | | Alcohol dehydrogenase, class III |
| 413 | | | |
| 414 | Transcription | MYB | Transcription factor, Myb superfamily |
| | Factor | | |
| 415 | | | UDP-glucuronosyl and UDP-glucosyl |
| | | | transferase |
| 416 | | | |
| 417 | | | |
| 418 | Transcription | Basic Helix- | |
| | Factor | Loop-Helix | |
| | | (bHLH) | |
| 419 | | | |
| 420 | | | Methyltransferase |
| 421 | Protein | | Serine/threonine protein phosphatase |
| | Phosphatase | | |
| 422 | | | Asparaginyl peptidases |
| 423 | | | |
| 424 | Transcription | AUX/IAA | |
| | Factor | | |
| 425 | | | |
| 426 | | | |
| 427 | | | Alanine-glyoxylate aminotransferase AGT2 |
| 428 | | | |
| 429 | Storage | | |
| | Protein | | |
| 430 | | | |
| 431 | | | |
| 432 | | | Hydroxyindole-O-methyltransferase and |
| | | | related SAM-dependent methyltransferases |
| 433 | | | |
| 434 | | | Iron/ascorbate family oxidoreductases |
| 435 | Sucrose | | Glycosyltransferase |
| | Biosynthesis | | |
| 436 | | | Aquaporin (major intrinsic protein family) |
| 437 | | | |
| 438 | | | |
| 439 | | | |
| 440 | | | |
| 441 | | | Purple (tartrate-resistant) acid phosphatase |
| 442 | | | Haloacid dehalogenase-like hydrolase |
| 443 | Protein | | Serine/threonine protein phosphatase |
| | Phosphatase | | |
| 444 | | | |
| 445 | Transcription | Zinc Finger | |
| | Factor | | |
| 446 | | | |
| 447 | Hormone | | |
| | Induced | | |
| 448 | | | |
| 449 | | | Predicted Yippee-type zinc-binding protein |
| 450 | | | |
| 451 | | | |
| 452 | | | |
| 453 | | | |
| 454 | | | H+/oligopeptide symporter |
| 455 | | | Arylacetamide deacetylase |
| 456 | | | |
| 457 | | | Tomosyn and related SNARE-interacting |
| | | | proteins |
| 458 | | | O-methyltransferase |
| 459 | | | |
| 460 | | | |
| 461 | | | Possible pfkB family carbohydrate kinase |
| 462 | | | |
| 463 | Transcription | MYB | Transcription factor, Myb superfamily |
| | Factor | | |
| 464 | | | Permease of the major facilitator |
| | | | superfamily |
| 465 | | | |
| 466 | | | |
| 467 | | | |
| 468 | Transcription | MYB | Transcription factor, Myb superfamily |
| | Factor | | |
| 469 | | | |
| 470 | | | |
| 471 | | | |
| 472 | | | |
| 473 | | | Arylacetamide deacetylase |
| 474 | | | Synaptic vesicle transporter SVOP and |
| | | | related transporters (major facilitator |
| | | | superfamily) |
| 475 | | | Sodium sulfate symporter and related |
| | | | arsenite permeases |
| 476 | | | Haloacid dehalogenase-like hydrolase |
| 477 | | | |
| 478 | Transcription | MYB | Transcription factor, Myb superfamily |
| | Factor | | |
| 479 | Transcription | MYB | Transcription factor, Myb superfamily |
| | Factor | | |
| 480 | | | |
| 481 | | | |
| 482 | | | |
| 483 | | | |
| 484 | Protein | | |
| | Phosphatase | | |
| 485 | | | |
| 486 | | | |
| 487 | Transcription | Heat Shock | Heat shock transcription factor |
| | Factor | | |
| 488 | | | Homocysteine S-methyltransferase |
| 489 | | | Iron/ascorbate family oxidoreductases |
| 490 | | | |
| 491 | | | |
| 492 | | | Uncharacterized conserved protein |
| 493 | | | Glutathione S-transferase |
| 494 | | | Possible pfkB family carbohydrate kinase |
| 495 | | | |
| 496 | | | |
| 497 | | | Endoplasmic reticulum protein EP58, |
| | | | contains filamin rod domain and KDEL |
| | | | motif |
| 498 | Protein | | |
| | Phosphatase | | |
| 499 | | | |
| 500 | | | Arylacetamide deacetylase |
| 501 | | | |
| 502 | | | Flavonol reductase/cinnamoyl-CoA |
| | | | reductase |
| 503 | Transcription | Homeodomain | Transcription factor MEIS1 and related |
| | Factor | | HOX domain proteins |
| 504 | | | |
| 505 | | | Uncharacterized conserved protein |
| 506 | | | Predicted haloacid dehalogenase-like |
| | | | hydrolase |
| 507 | | | |
| 508 | Transcription | NAC Domain | |
| | Factor | | |
| 509 | GA | | Iron/ascorbate family oxidoreductases |
| | Biosynthesis | | |
| 510 | Transcription | NAC Domain | |
| | Factor | | |
| 511 | | | |
| 512 | | | |
| 513 | | | |
| 514 | | | Flavin-containing monooxygenase |
| 515 | Transcription | CCAAT | CCAAT-binding factor, subunit B (HAP2) |
| | Factor | | |
| 516 | | | |
| 517 | | | |
| 518 | | | |
| 519 | | | |
| 520 | Unclassified | | Phosphatidylethanolamine binding protein |
| 521 | Cytochrome | CYP81D | Cytochrome P450 CYP2 subfamily |
| | P450 | | |
| 522 | | | |
| 523 | | | |
| 524 | Transcription | CCAAT | CCAAT-binding factor, subunit A (HAP3) |
| | Factor | | |
| 525 | | | |
| 526 | | | |
| 527 | | | |
| 528 | | | Phenylalanine and histidine ammonia-lyase |
| 529 | Transcription | Homeodomain | Transcription factor HEX, contains HOX |
| | Factor | | and HALZ domains |
| 530 | | | |
| 531 | | | |
| 532 | | | Sodium sulfate symporter and related |
| | | | arsenite permeases |
| 533 | | | |
| 534 | | | 5′-AMP-activated protein kinase, gamma |
| | | | subunit |
| 535 | | | |
| 536 | Ethylene | | Iron/ascorbate family oxidoreductases |
| | Biosynthesis | | |
| 537 | | | Flavonol reductase/cinnamoyl-CoA |
| | | | reductase |
| 538 | | | |
| 539 | | | Sulfotransferase |
| 540 | Protein | | Serine/threonine protein phosphatase |
| | Phosphatase | | |
| 541 | | | |
| 542 | | | |
| 543 | Transcription | Basic Helix- | |
| | Factor | Loop-Helix | |
| | | (bHLH) | |
| 544 | | | Predicted chitinase |
| 545 | | | |
| 546 | | | |
| 547 | | | |
| 548 | | | |
| 549 | | | |
| 550 | | | Aspartyl protease |
| 551 | | | Hydroxyindole-O-methyltransferase and |
| | | | related SAM-dependent methyltransferases |
| 552 | | | Protein involved in thiamine biosynthesis |
| | | | and DNA damage tolerance |
| 553 | | | Flavonol reductase/cinnamoyl-CoA |
| | | | reductase |
| 554 | | | |
| 555 | | | Sulfotransferase |
| 556 | | | |
| 557 | | | |
| 558 | | | |
| 559 | | | Acyl-CoA synthetase |
| 560 | | | |
| 561 | | | Predicted transporter (major facilitator |
| | | | superfamily) |
| 562 | | | Permease of the major facilitator |
| | | | superfamily |
| 563 | | | |
| 564 | | | |
| 565 | | | Glucose-6-phosphate/phosphate and |
| | | | phosphoenolpyruvate/phosphate antiporter |
| 566 | | | |
| 567 | Protein | | Serine/threonine protein kinase |
| | Kinase | | |
| 568 | | | Selenium-binding protein |
| 569 | Protein | | Serine/threonine protein phosphatase |
| | Phosphatase | | |
| 570 | | | |
| 571 | | | Aquaporin (major intrinsic protein family) |
| 572 | | | |
| 573 | | | |
| 574 | | | Glyoxylate/hydroxypyruvate reductase (D- |
| | | | isomer-specific 2-hydroxy acid |
| | | | dehydrogenase superfamily) |
| 575 | Protein | | Serine/threonine protein kinase |
| | Kinase | | |
| 576 | | | Predicted membrane protein |
| 577 | | | |
| 578 | | | |
| 579 | | | Hydroxyindole-O-methyltransferase and |
| | | | related SAM-dependent methyltransferases |
| 580 | | | Acyl-CoA synthetase |
| 581 | | | |
| 582 | Sulfur | | |
| | Metabolism | | |
| 583 | | | |
| 584 | | | Flavonol reductase/cinnamoyl-CoA |
| | | | reductase |
| 585 | | | |
| 586 | Transcription | Heat Shock | Heat shock transcription factor |
| | Factor | | |
| 587 | | | |
| 588 | | | |
| 589 | | | |
| 590 | | | |
| 591 | | | Alanine-glyoxylate aminotransferase AGT2 |
| 592 | | | |
| 593 | | | |
| 594 | | | |
| 595 | | | |
| 596 | | | Thioredoxin, nucleoredoxin and related |
| | | | proteins |
| 597 | | | Reductases with broad range of substrate |
| | | | specificities |
| 598 | | | |
| 599 | | | |
| 600 | | | |
| 601 | | | |
| 602 | | | |
| 603 | | | |
| 604 | | | Beta, beta-carotene 15,15′-dioxygenase and |
| | | | related enzymes |
| 605 | | | |
| 606 | | | |
| 607 | | | Beta, beta-carotene 15,15′-dioxygenase and |
| | | | related enzymes |
| 608 | | | |
| 609 | | | Molecular chaperones HSP70/HSC70, |
| | | | HSP70 superfamily |
| 610 | | | |
| 611 | | | |
| 612 | | | Fumarase |
| 613 | | | |
| 614 | | | |
| 615 | Amino Acid | | Asparagine synthase (glutamine- |
| | Biosynthesis/ | | hydrolyzing) |
| | Metabolism | | |
| 616 | | | |
| 617 | | | |
| 618 | | | 5′-AMP-activated protein kinase, gamma |
| | | | subunit |
| 619 | | | |
| 620 | | | |
| 621 | Amino Acid | | Asparagine synthase (glutamine- |
| | Biosynthesis/ | | hydrolyzing) |
| | Metabolism | | |
| 622 | | | |
| 623 | | | |
| 624 | | | 5′-AMP-activated protein kinase, gamma |
| | | | subunit |
| 625 | | | |
| 626 | Storage | | Uncharacterized conserved protein |
| | Protein | | |
| 627 | | | |
| 628 | | | 5′-AMP-activated protein kinase, gamma |
| | | | subunit |
| 629 | | | |
| 630 | | | |
| 631 | | | Ca2+/H+ antiporter VCX1 and related |
| | | | proteins |
| 632 | | | Flavonol reductase/cinnamoyl-CoA |
| | | | reductase |
| 633 | Storage | | |
| | Protein | | |
| 634 | Ethylene | | 1-aminocyclopropane-1-carboxylate |
| | Biosynthesis | | synthase, and related proteins |
| 635 | | | |
| 636 | Cytochrome | CYP93D | Cytochrome P450 CYP2 subfamily |
| | P450 | | |
| 637 | | | |
| 638 | Storage | | Uncharacterized conserved protein |
| | Protein | | |
| 639 | | | |
| 640 | | | |
| 641 | | | Gamma-glutamyl hydrolase |
| 642 | | | |
| 643 | | | |
| 644 | | | |
| 645 | | | Thioredoxin, nucleoredoxin and related |
| | | | proteins |
| 646 | | | |
| 647 | Stress | | |
| | Induced | | |
| 648 | | | |
| 649 | | | Thioredoxin, nucleoredoxin and related |
| | | | proteins |
| 650 | | | |

TABLE 5

| | | Fold | | | | | |
|---|---|---|---|---|---|---|---|
| ID | Gene Name | Change | Soybean_ GenBank_Acc | Functional Category | Sub-Family | Gene Family | Additional Description |
| 1 | Gma.4058.1.S1_at | 5.006 | AW310338 | Transporter | | | Predicted transporter (major |
| | | | | | | | facilitator superfamily) |
| 2 | Gma.8301.2.S1_at | 3.484 | BU544524 | Signal Transduction | Calcium Binding | | |
| 3 | Gma.8416.1.S1_at | 3.472 | U13987.1 | Metabolism | | | Cytochrome b5 |
| 3 | Gma.8416.1.S1_at | 3.472 | U13987.1 | Metabolism | | | NADH-cytochrome b-5 |
| | | | | | | | reductase |
| 3 | Gma.8416.1.S1_at | 3.472 | U13987.1 | Metabolism | | | Sulfite oxidase, molybdopterin- |
| | | | | | | | binding component |
| 4 | GmaAffx.66589.1.S1_at | 3.45 | BE821501 | Transcription | Transcription | Polycomb Group | |
| | | | | | Factor | | |
| 5 | Gma.1221.1.S1_s_at | 3.365 | L23854.1 | Metabolism | | | Cytochrome b5 |
| 5 | Gma.1221.1.S1_s_at | 3.365 | L23854.1 | Metabolism | | | NADH-cytochrome b-5 |
| | | | | | | | reductase |
| 5 | Gma.1221.1.S1_s_at | 3.365 | L23854.1 | Metabolism | | | Sulfite oxidase, molybdopterin- |
| | | | | | | | binding component |
| 6 | Gma.10921.1.S1_s_at | 3.348 | BQ611250 | No Homology to | | | |
| | | | | Known Proteins | | | |
| 7 | GmaAffx.62542.1.S1_at | 3.33 | BE659136 | Unclassified - Hypothetical | | | |
| | | | | Protein NOT Supported by | | | |
| | | | | cDNA | | | |
| 8 | GmaAffx.67477.2.S1_at | 3.177 | AW509184 | Cell Structure | | | |
| 9 | GmaAffx.19116.1.S1_at | 3.012 | BI968419 | Signal Transduction | Protein Kinase | | |
| 10 | Gma.10307.1.S1_at | 2.93 | CA936845 | Unclassified - Hypothetical | | | |
| | | | | Protein NOT Supported by | | | |
| | | | | cDNA | | | |
| 11 | Gma.12356.1.A1_at | 2.882 | CD397798 | Unclassified - Hypothetical | | | |
| | | | | Protein NOT Supported by | | | |
| | | | | cDNA | | | |
| 12 | GmaAffx.13179.1.S1_at | 2.769 | CD402274 | Unclassified - Hypothetical | | | |
| | | | | Protein NOT Supported by | | | |
| | | | | cDNA | | | |
| 13 | Gma.6266.1.A1_at | 2.64 | CD413418 | Unclassified - Protein with | | | |
| | | | | Unknown Function | | | |
| 14 | Gma.5689.3.S1_s_at | 2.631 | BE657645 | Protein Destination & Storage | | | Gelatinase A and related matrix |
| | | | | | | | metalloproteases |
| 15 | Gma.2194.1.S1_at | 2.524 | BE657352 | Unclassified - Hypothetical | | | |
| | | | | Protein Supported by | | | |
| | | | | cDNA | | | |
| 16 | Gma.17789.1.S1_at | 2.408 | BG511704 | Unclassified - Hypothetical | | | |
| | | | | Protein Supported by | | | |
| | | | | cDNA | | | |
| 17 | GmaAffx.90042.1.S1_at | 2.372 | CF805767 | Signal Transduction | Calcium Binding | | Calmodulin and related proteins |
| | | | | | | | (EF-Hand s uperfamily) |
| 18 | GmaAffx.30420.1.S1_at | 2.207 | AW704927 | Transporter | | | |
| 19 | Gma.14802.1.S1_at | 2.141 | AW201116 | Unclassified - Protein with | | | FOG: Ankyrin repeat |
| | | | | Unknown Function | | | |
| 20 | Gma.964.1.A1_at | 2.128 | BE824146 | Metabolism | | | Tyrosine aminotransferase |
| 21 | Gma.869.1.S1_at | 2.125 | BE822531 | Protein Destination & Storage | | | Serine carboxypeptidases |
| | | | | | | | (lysosomal cathepsin A) |
| 22 | GmaAffx.93462.1.S1_s_at | 2.116 | CF809187 | Signal Transduction | Protein Kinase | AtCIPKs | Serine/threonine protein kinase |
| 23 | GmaAffx.31180.1.A1_at | 2.113 | BE820254 | No Homology to | | | |
| | | | | Known Proteins | | | |
| 24 | GmaAffx.56804.1.A1_at | 2.111 | CD415337 | Unclassified - Hypothetical | | | Uncharacterized conserved |
| | | | | Protein NOT Supported by | | | protein, contains TraB domain |
| | | | | cDNA | | | |
| 25 | GmaAffx.83103.2.S1_at | 2.072 | BE806704 | Unclassified - Hypothetical | | | |
| | | | | Protein NOT Supported by | | | |
| | | | | cDNA | | | |
| 26 | GmaAffx.87806.1.S1_at | 2.032 | BG882829 | Unclassified - Hypothetical | | | |
| | | | | Protein NOT Supported by | | | |
| | | | | cDNA | | | |
| 27 | Gma.4701.2.A1_a_at | 2.011 | BM526048 | Energy | | | Thioredoxin |
| 28 | Gma.10787.2.S1_a_at | 0.5 | BE023083 | Transcription | Transcription | Squamosa | |
| | | | | | Factor | | |
| 29 | GmaAffx.84721.1.S1_at | 0.5 | BQ297863 | Metabolism | | | Hydroxymethylglutaryl-CoA |
| | | | | | | | lyase |
| 30 | GmaAffx.90970.1.S1_s_at | 0.5 | CF806695 | Unclassified - Hypothetical | | | |
| | | | | Protein Supported by | | | |
| | | | | cDNA | | | |
| 31 | Gma.12821.1.S1_s_at | 0.499 | CD391504 | No Homology to | | | |
| | | | | Known Proteins | | | |
| 32 | GmaAffx.71386.1.S1_at | 0.499 | AW704747 | Protein Destination & Storage | | | Molecular chaperone |
| | | | | | | | (DnaJ superfamily) |
| 33 | GmaAffx.23289.1.S1_at | 0.499 | CD392087 | No Homology to | | | |
| | | | | Known Proteins | | | |
| 34 | Gma.8782.1.A1_at | 0.499 | AW309953 | Disease & Defense | | | Apoptotic ATPase |
| 35 | Gma.17191.1.S1_at | 0.499 | BG237724 | Metabolism | | | UDP-glucuronosyl and UDP- |
| | | | | | | | glucosyl transferase |
| 36 | GmaAffx.72346.1.S1_at | 0.499 | BF424359 | Unclassified - Hypothetical | | | Speckle-type POZ protein SPOP and |
| | | | | Protein NOT Supported by | | | related proteins with TRAF, MATH |
| | | | | cDNA | | | and BTB/POZ domains |
| 37 | GmaAffx.77527.1.S1_at | 0.499 | BF423510 | Unclassified - Hypothetical | | | GTPase Rab18, small G protein |
| | | | | Protein Supported by | | | superfamily |
| | | | | cDNA | | | |
| 38 | GmaAffx.87922.1.S1_at | 0.499 | BU084547 | Energy | | | Pyrophosphate-dependent |
| | | | | | | | phosphofructo-1-kinase |
| 39 | GmaAffx.65393.1.S1_s_at | 0.499 | BU760770 | No Homology to | | | |
| | | | | Known Proteins | | | |
| 40 | GmaAffx.75708.1.S1_at | 0.497 | BF599014 | Unclassified - Hypothetical | | | |
| | | | | Protein Supported by | | | |
| | | | | cDNA | | | |
| 41 | Gma.6025.1.S1_at | 0.497 | CD399734 | Unclassified - Hypothetical | | | |
| | | | | Protein NOT Supported by | | | |
| | | | | cDNA | | | |
| 42 | GmaAffx.36972.2.S1_at | 0.496 | BI699120 | Unclassified - Hypothetical | | | |
| | | | | Protein NOT Supported by | | | |
| | | | | cDNA | | | |
| 43 | Gma.6528.2.S1_at | 0.496 | CA784209 | Transporter | | | Calcium transporting ATPase |
| 44 | Gma.7835.1.S1_at | 0.496 | CD410897 | Cell Growth & Division | | | |
| 45 | GmaAffx.6565.1.A1_at | 0.496 | CD402930 | No Homology to | | | |
| | | | | Known Proteins | | | |
| 46 | GmaAffx.71778.1.S1_at | 0.495 | BE346999 | Unclassified - Hypothetical | | | |
| | | | | Protein NOT Supported by | | | |
| | | | | cDNA | | | |
| 47 | GmaAffx.1128.1.S1_at | 0.495 | AW348608 | Unclassified - Protein with | | | Copper chaperone |
| | | | | Unknown Function | | | |
| 48 | GmaAffx.53585.2.S1_at | 0.495 | BE556399 | Protein Destination & Storage | | | Molecular chaperone |
| | | | | | | | (DnaJ superfamily) |
| 49 | GmaAffx.37484.2.S1_at | 0.494 | BQ629925 | Unclassified - Protein with | | | Predicted membrane protein, |
| | | | | Unknown Function | | | contains DoH and Cytochrome |
| | | | | | | | b-561/ferric reductase |
| | | | | | | | transmembrane domains |
| 50 | Gma.14155.3.S1_at | 0.494 | AI437500 | Unclassified - Hypothetical | | | Clathrin assembly protein |
| | | | | Protein Supported by | | | API80 and related proteins, |
| | | | | cDNA | | | contain ENTH domain |
| 51 | Gma.1677.2.A1_at | 0.494 | AW348386 | Secondary Metabolism | | | |
| 52 | GmaAffx.50632.1.A1_at | 0.494 | BI969115 | Metabolism | | | Malate dehydrogenase |
| 53 | Gma.6722.1.S1_at | 0.494 | BE822185 | Unclassified - Hypothetical | | | UDP-glucuronosyl and UDP- |
| | | | | Protein NOT Supported by | | | glucosyl transferase |
| | | | | cDNA | | | |
| 54 | GmaAffx.62195.1.S1_at | 0.493 | AW472439 | No Homology to | | | |
| | | | | Known Proteins | | | |
| 55 | GmaAffx.11383.1.S1_at | 0.493 | BE822014 | Unclassified - Hypothetical | | | Kinesin light chain |
| | | | | Protein NOT Supported by | | | |
| | | | | cDNA | | | |
| 56 | GmaAffx.77651.1.S1_at | 0.493 | BF010272 | Unclassified - Protein with | Hormone Induced | | Zinc-binding oxidoreductase |
| | | | | Unknown Function | | | |
| 57 | Gma.9717.1.S1_at | 0.492 | BG653084 | Unclassified - Hypothetical | | | Amino acid transporters |
| | | | | Protein NOT Supported by | | | |
| | | | | cDNA | | | |
| 58 | GmaAffx.21553.1.S1_at | 0.492 | BE023327 | Protein Destination & Storage | | | Gelatinase A and related matrix |
| | | | | | | | metalloproteases |
| 59 | GmaAffx.90952.1.S1_at | 0.491 | CF806677 | Signal Transduction | Unclassified | | |
| 60 | Gma.3175.1.S1_at | 0.491 | AI736154 | Unclassified - Hypothetical | | | |
| | | | | Protein NOT Supported by | | | |
| | | | | cDNA | | | |
| 61 | GmaAffx.84871.1.S1_at | 0.491 | BF599103 | Signal Transduction | Protein Kinase | | |
| 62 | GmaAffx.56020.1.S1_at | 0.49 | BU080527 | Unclassified - Hypothetical | | | |
| | | | | Protein NOT Supported by | | | |
| | | | | cDNA | | | |
| 63 | GmaAffx.765.2.S1_at | 0.49 | AW509310 | Transporter | | | Amino acid transporters |
| 64 | GmaAffx.86283.1.S1_at | 0.49 | BI892496 | Unclassified - Hypothetical | | | Predicted pyroglutamyl peptidase |
| | | | | Protein Supported by | | | |
| | | | | cDNA | | | |
| 65 | Gma.9436.1.S1_at | 0.49 | CA802734 | Unclassified - Hypothetical | | | |
| | | | | Protein NOT Supported by | | | |
| | | | | cDNA | | | |
| 66 | Gma.15155.2.S1_a_at | 0.489 | BG789619 | Unclassified - Hypothetical | | | |
| | | | | Protein Supported by | | | |
| | | | | cDNA | | | |
| 67 | Gma.16441.1.S1_a_at | 0.489 | BU548048 | Unclassified - Hypothetical | | | HGG motif-containing thioesterase |
| | | | | Protein NOT Supported by | | | |
| | | | | cDNA | | | |
| 68 | Gma.1058.1.S1_at | 0.489 | BE820580 | Metabolism | | | |
| 69 | Gma.17305.1.S1_at | 0.489 | AW351319 | Unclassified - Protein with | | | |
| | | | | Unknown Function | | | |
| 70 | Gma.15729.1.A1_at | 0.489 | BI971055 | Cell Growth & Division | | | |
| 71 | GmaAffx.11840.1.S1_at | 0.488 | AW349211 | Post- Transcription | | | Predicted E3 ubiquitin ligase |
| 72 | GmaAffx.60958.3.S1_at | 0.487 | BG881523 | No Homology to | | | |
| | | | | Known Proteins | | | |
| 73 | GmaAffx.77269.1.S1_at | 0.487 | BM093698 | No Homology to | | | |
| | | | | Known Proteins | | | |
| 74 | Gma.7120.1.S1_at | 0.486 | BM093481 | Metabolism | | | Alanine-glyoxylate |
| | | | | | | | aminotransferaseAGT2 |
| 75 | GmaAffx.6818.1.A1_at | 0.486 | BU546572 | Metabolism | | | Iron/ascorbate family |
| | | | | | | | oxidoreductases |
| 76 | GmaAffx.80638.1.S1_at | 0.486 | AW396070 | Unclassified - Hypothetical | | | |
| | | | | Protein NOT Supported by | | | |
| | | | | cDNA | | | |
| 77 | Gma.5621.1.S1_at | 0.485 | BU579234 | Unclassified - Hypothetical | | | |
| | | | | Protein NOT Supported by | | | |
| | | | | cDNA | | | |
| 78 | GmaAffx.93592.1.S1_s_at | 0.485 | CF809317 | Unclassified - Hypothetical | | | |
| | | | | Protein Supported by | | | |
| | | | | cDNA | | | |
| 79 | GmaAffx.77190.1.S1_at | 0.485 | BQ741890 | Metabolism | | | |
| 80 | GmaAffx.67456.1.S1_at | 0.485 | BM893057 | No Homology to | | | |
| | | | | Known Proteins | | | |
| 81 | GmaAffx.89049.1.S1_s_at | 0.485 | CK606168 | Intracellular Traffic | | | Annexin |
| 82 | GmaAffx.11419.1.S1_at | 0.485 | AI900831 | No Homology to | | | |
| | | | | Known Proteins | | | |
| 83 | GmaAffx.81692.1.S1_at | 0.484 | CA937041 | No Homology to | | | |
| | | | | Known Proteins | | | |
| 84 | Gma.197.1.S1_at | 0.484 | U55874.1 | Metabolism | Amino Acid Biosynthesis/ | | Asparagine synthase |
| | | | | | Metabolism | | (glutamine-hydrolyzing) |
| 85 | GmaAffx.87550.1.S1_at | 0.484 | BM954718 | Disease & Defense | | | |
| 86 | Gma.6803.2.S1_s_at | 0.483 | BU082740 | Disease & Defense | | | |
| 87 | GmaAffx.78790.1.S1_at | 0.483 | BQ298762 | Transporter | | | P-type ATPase |
| 88 | GmaAffx.91338.1.S1_at | 0.483 | CF807063 | Unclassified - Hypothetical | | | Predicted haloacid |
| | | | | Protein Supported by | | | dehalogenase-like hydrolase |
| | | | | cDNA | | | |
| 89 | GmaAffx.93534.1.S1_s_at | 0.483 | CF809259 | Unclassified - Protein with | | | |
| | | | | Unknown Function | | | |
| 90 | GmaAffx.47788.1.A1_at | 0.483 | CD408318 | Metabolism | | | Aldehyde dehydrogenase |
| 91 | Gma.10857.2.S1_s_at | 0.483 | BQ612778 | Unclassified - Hypothetical | | | Fasciclin and related adhesion |
| | | | | Protein Supported by | | | glycoproteins |
| | | | | cDNA | | | |
| 92 | GmaAffx.87721.1.S1_at | 0.483 | AW156448 | Intracellular Traffic | | | Rho GDP-dissociation |
| | | | | | | | inhibitor |
| 93 | Gma.13613.1.A1_at | 0.482 | CD401987 | No Homology to | | | |
| | | | | Known Proteins | | | |
| 94 | Gma.9013.1.S1_x_at | 0.482 | AF243368.1 | Disease & Defense | | | Glutathione |
| | | | | | | | S-transferase |
| 95 | Gma.3400.1.S1_at | 0.482 | BQ627988 | No Homology to | | | |
| | | | | Known Proteins | | | |
| 96 | Gma.10940.1.S1_at | 0.482 | BU550016 | Unclassified - Hypothetical | | | Metalloendopeptidase family - |
| | | | | Protein Supported by | | | saccharolysin & thimet |
| | | | | cDNA | | | oligopeptidase |
| 97 | GmaAffx.53033.1.S1_at | 0.482 | BG046837 | Transcription | Transcription | AP2/EREBP | |
| | | | | | Factor | | |
| 98 | GmaAffx.82263.1.S1_s_at | 0.482 | AW568454 | No Homology to | | | |
| | | | | Known Proteins | | | |
| 99 | Gma.5816.I.S1_at | 0.481 | BI787790 | Unclassified - Hypothetical | | | Aldehyde reductase |
| | | | | Protein Supported by | | | |
| | | | | cDNA | | | |
| 100 | Gma.2771.1.S1_at | 0.481 | AW309763 | Unclassified - Hypothetical | | | |
| | | | | Protein Supported by | | | |
| | | | | cDNA | | | |
| 101 | Gma.10701.2.S1_at | 0.481 | AW101512 | No Homology to | | | |
| | | | | Known Proteins | | | |
| 102 | GmaAffx.93260.1.S1_at | 0.481 | CF808985 | Signal Transduction | Protein Kinase | | |
| 103 | GmaAffx.53585.1.S1_at | 0.481 | BI471867 | Protein Destination & Storage | | | Molecular chaperone |
| | | | | | | | (DnaJ superfamily) |
| 104 | GmaAffx.77622.1.S1_at | 0.48 | BI674271 | Transcription | Transcription | Zinc Finger | |
| | | | | | Factor | | |
| 105 | GmaAffx.74868.1.S1_at | 0.48 | CA820573 | No Homology to | | | |
| | | | | Known Proteins | | | |
| 106 | GmaAffx.2412.1.S1_at | 0.48 | AI961002 | Secondary Metabolism | GA Biosynthesis | | Iron/ascorbate family |
| | | | | | | | oxidoreductases |
| 107 | GmaAffx.88251.1.S1_s_at | 0.48 | BU964415 | Disease & Defense | | | Soluble epoxide hydrolase |
| 108 | GmaAffx.68318.1.S1_at | 0.48 | BU545357 | Transcription | Transcription | AUX/IAA | |
| | | | | | Factor | | |
| 109 | Gma.17798.1.S1_at | 0.479 | BU084728 | Unclassified - Hypothetical | | | |
| | | | | Protein Supported by | | | |
| | | | | cDNA | | | |
| 110 | Gma.11334.1.S1_a_at | 0.479 | BI967327 | Cell Structure | | | O-methyltransferase |
| 111 | GmaAffx.49130.1.S1_at | 0.479 | BE555035 | No Homology to | | | |
| | | | | Known Proteins | | | |
| 112 | GmaAffx.37484.1.S1_at | 0.479 | BG651852 | Unclassified - Protein with | | | Predicted membrane protein, contains |
| | | | | Unknown Function | | | DoH and Cytochrome b-561/ferric |
| | | | | | | | reductase transmembrane domains |
| 113 | Gma.11341.3.S1_s_at | 0.479 | BM177332 | Unclassified - Hypothetical | | | |
| | | | | Protein NOT Supported by | | | |
| | | | | cDNA | | | |
| 114 | GmaAffx.47590.1.S1_at | 0.478 | CA853861 | Metabolism | | | UDP-glucuronosyl and UDP- |
| | | | | | | | glucosyl transferase |
| 115 | GmaAffx.499.1.S1_at | 0.478 | BE058375 | Transcription | Transcription | MYB | Transcription factor, Myb |
| | | | | | Factor | | superfamily |
| 116 | GmaAffx.90097.1.S1_at | 0.477 | CF805822 | Cell Structure | | | |
| 117 | Gma.15499.1.S1_at | 0.477 | AW348149 | Energy | | | |
| 118 | GmaAffx.83232.1.S1_at | 0.477 | BE023128 | Disease & Defense | | | |
| 119 | Gma.6075.1.S1_a_at | 0.476 | AW317183 | Disease & Defense | | | |
| 120 | Gma.3160.1.S1_at | 0.476 | BG507816 | Energy | | | Thioredoxin |
| 121 | Gma.8525.1.S1_s_at | 0.476 | AI966178 | Disease & Defense | | | |
| 122 | Gma.18052.1.S1_at | 0.476 | BF595464 | Transporter | | | |
| 123 | Gma.11672.1.S1_at | 0.476 | BF425929 | Unclassified - Hypothetical | | | |
| | | | | Protein Supported by | | | |
| | | | | cDNA | | | |
| 124 | Gma.385.1.S1_at | 0.475 | BE347563 | Intracellular Traffic | | | Annexin |
| 125 | Gma.11135.1.S1_at | 0.475 | AI856724 | Energy | | | ATP-citrate lyase |
| 126 | Gma.17070.1.S1_at | 0.475 | BE822463 | Disease & Defense | | | O-methyltransferase |
| 127 | Gma.1445.1.S1_a_at | 0.475 | BQ454241 | Metabolism | | | Flavonol reductase/cinnamoyl-CoA |
| | | | | | | | reductase |
| 128 | GmaAffx.26908.1.S1_at | 0.475 | BF425834 | Secondary Metabolism | | | Beta, beta-carotene 15,15′- |
| | | | | | | | dioxygenase and related enzymes |
| 129 | GmaAffx.35729.2.S1_at | 0.474 | BM732483 | Unclassified - Hypothetical | | | |
| | | | | Protein Supported by | | | |
| | | | | cDNA | | | |
| 130 | GmaAffx.77832.1.S1_at | 0.474 | BI971626 | Unclassified - Hypothetical | | | |
| | | | | Protein NOT Supported by | | | |
| | | | | cDNA | | | |
| 131 | Gma.13257.1.A1_at | 0.474 | CD396346 | Unclassified - Protein with | | | |
| | | | | Unknown Function | | | |
| 132 | Gma.9228.1.S1_at | 0.473 | CD417435 | Unclassified - Hypothetical | | | |
| | | | | Protein NOT Supported by | | | |
| | | | | cDNA | | | |
| 133 | Gma.1749.3.S1_at | 0.472 | BI784667 | Protein Destination & Storage | Ubiquitin Degradation | | FOG: Predicted E3 |
| | | | | | | | ubiquitin ligase |
| 134 | Gma.4643.1.S1_at | 0.472 | BU547906 | Transcription | Transcription | NAC Domain | |
| | | | | | Factor | | |
| 135 | GmaAffx.93212.1.S1_x_at | 0.472 | CF807524 | Transporter | | | |
| 136 | GmaAffx.45853.1.S1_at | 0.471 | BQ081229 | Unclassified - Protein with | | | |
| | | | | Unknown Function | | | |
| 137 | Gma.14563.1.S1_s_at | 0.471 | CD399224 | Energy | | | |
| 138 | GmaAffx.74500.1.A1_at | 0.471 | BU551117 | No Homology to | | | |
| | | | | Known Proteins | | | |
| 139 | Gma.16288.1.S1_at | 0.471 | AW598590 | Unclassified - Hypothetical | | | Fasciclin and related |
| | | | | Protein NOT Supported by | | | adhesion glycoproteins |
| | | | | cDNA | | | |
| 140 | GmaAffx.85342.1.S1_at | 0.471 | BI426916 | Unclassified - Hypothetical | | | |
| | | | | Protein NOT Supported by | | | |
| | | | | cDNA | | | |
| 141 | GmaAffx.24352.1.S1_at | 0.47 | BU544287 | No Homology to | | | |
| | | | | Known Proteins | | | |
| 142 | GmaAffx.15471.3.S1_s_at | 0.47 | BE346777 | Transcription | Transcription | MYB | Transcription factor, Myb |
| | | | | | Factor | | superfamily |
| 143 | Gma.17861.1.S1_at | 0.47 | AW310110 | Cell Structure | | | |
| 144 | Gma.1294.1.S1_at | 0.47 | CD395653 | Unclassified - Hypothetical | | | |
| | | | | Protein Supported by | | | |
| | | | | cDNA | | | |
| 145 | GmaAffx.36017.1.S1_at | 0.469 | BG154593 | Unclassified - Hypothetical | | | |
| | | | | Protein Supported by | | | |
| | | | | cDNA | | | |
| 146 | GmaAffx.83638.1.S1_at | 0.469 | BU549896 | Unclassified - Hypothetical | | | |
| | | | | Protein NOT Supported by | | | |
| | | | | cDNA | | | |
| 147 | Gma.2470.1.S1_s_at | 0.468 | AI442629 | Cell Structure | | | |
| 148 | GmaAffx.91514.1.S1_at | 0.468 | CF807239 | No Homology to | | | |
| | | | | Known Proteins | | | |
| 149 | GmaAffx.28690.1.S1_at | 0.467 | CA799411 | Transcription | Transcription | Homeo domain | |
| | | | | | Factor | | |
| 150 | Gma.17763.1.A1_at | 0.467 | BI321757 | Unclassified - Hypothetical | | | Diadenosine and diphosphoinositol |
| | | | | Protein Supported by | | | polyphosphate phosphohydrolase |
| | | | | cDNA | | | |
| 151 | Gma.6659.1.S1_at | 0.467 | BQ629137 | Unclassified - Protein with | | | Protein involved in |
| | | | | Unknown Function | | | membrane traffic |
| | | | | | | | (YOP1/TB2/DP1/HVA22 family) |
| 152 | Gma.8143.1.S1_at | 0.466 | BG725425 | Signal Transduction | MAPK | | Mitogen-activated |
| | | | | | | | protein kinase |
| 153 | GmaAffx.68099.1.S1_at | 0.466 | AW734055 | No Homology to | | | |
| | | | | Known Proteins | | | |
| 154 | Gma.3065.1.S1_s_at | 0.466 | AW349564 | Unclassified - Hypothetical | | | |
| | | | | Protein Supported by | | | |
| | | | | cDNA | | | |
| 155 | GmaAffx.87526.1.S1_at | 0.465 | BM954581 | Unclassified - Hypothetical | | | |
| | | | | Protein Supported by | | | |
| | | | | cDNA | | | |
| 156 | Gma.3881.1.S1_at | 0.465 | X92437.1 | Metabolism | Cytochrome P450 | CYP73A | Cytochrome P450 CYP2 |
| | | | | | | | subfamily |
| 157 | Gma.5468.2.S1_a_at | 0.465 | BE802025 | Metabolism | | | Beta-galactosidase |
| 158 | GmaAffx.87295.1.S1_at | 0.465 | BM519660 | No Homology to | | | |
| | | | | Known Proteins | | | |
| 159 | Gma.1701.1.S1_at | 0.464 | M67449.1 | Signal Transduction | MAPKKK | Raf | Tyrosine kinase specific for |
| | | | | | | | activated (GTP-bound) p21cdc42Hs |
| 160 | GmaAffx.3548.1.S1_at | 0.464 | CA851347 | Transporter | | | Apolipoprotein D/Lipocalin |
| 161 | Gma.12584.2.A1_at | 0.464 | CD406878 | Metabolism | | | Long-chain acyl-CoA |
| | | | | | | | synthetases (AMP-forming) |
| 162 | GmaAffx.8113.1.S1_at | 0.463 | BE659323 | Disease & Defense | | | |
| 163 | GmaAffx.64885.1.S1_at | 0.462 | BQ081653 | No Homology to | | | |
| | | | | Known Proteins | | | |
| 164 | Gma.1445.3.S1_a_at | 0.461 | BE805686 | Metabolism | | | Flavonol reductase/cinnamoyl-CoA |
| | | | | | | | reductase |
| 165 | GmaAffx.93308.1.S1_at | 0.461 | CF809033 | Protein Destination & Storage | | | Glutathione |
| | | | | | | | S-transferase |
| 166 | Gma.8072.1.S1_a_at | 0.461 | BQ627840 | Transcription | Transcription | Basic Leucine | |
| | | | | | Factor | Zipper (bZIP) | |
| 167 | GmaAffx.68588.1.S1_at | 0.46 | BI787152 | Unclassified - Hypothetical | | | |
| | | | | Protein Supported by | | | |
| | | | | cDNA | | | |
| 168 | GmaAffx.69369.1.S1_at | 0.46 | BG511072 | Unclassified - Hypothetical | | | Galactosyltransferases |
| | | | | Protein Supported by | | | |
| | | | | cDNA | | | |
| 169 | GmaAffx.21943.1.S1_at | 0.46 | AW101735 | Unclassified - Hypothetical | | | |
| | | | | Protein NOT Supported by | | | |
| | | | | cDNA | | | |
| 170 | GmaAffx.24382.1.S1_at | 0.46 | BM886764 | No Homology to | | | |
| | | | | Known Proteins | | | |
| 171 | GmaAffx.64794.1.S1_at | 0.46 | CA801749 | Unclassified - Hypothetical | | | |
| | | | | Protein Supported by | | | |
| | | | | cDNA | | | |
| 172 | GmaAffx.62941.1.S1_at | 0.459 | AW597498 | Unclassified - Protein with | | | |
| | | | | Unknown Function | | | |
| 173 | GmaAffx.80474.1.S1_at | 0.458 | AW707170 | Signal Transduction | MAPKKK | MEKK | MEKK and related serine/ |
| | | | | | | | threonine protein kinases |
| 174 | GmaAffx.59210.1.S1_at | 0.458 | BI967182 | Secondary Metabolism | Ethylene | | Iron/ascorbate family |
| | | | | | Biosynthesis | | oxidoreductases |
| 175 | GmaAffx.84721.2.S1_s_at | 0.458 | BG041982 | Metabolism | | | Hydroxymethylglutaryl-CoA |
| | | | | | | | lyase |
| 176 | GmaAffx.53069.1.S1_at | 0.458 | AW733700 | Unclassified - Hypothetical | | | |
| | | | | Protein NOT Supported by | | | |
| | | | | cDNA | | | |
| 177 | GmaAffx.69076.1.S1_at | 0.458 | BM525610 | Unclassified - Hypothetical | | | |
| | | | | Protein NOT Supported by | | | |
| | | | | cDNA | | | |
| 178 | GmaAffx.18236.1.S1_at | 0.458 | BG790288 | No Homology to | | | |
| | | | | Known Proteins | | | |
| 179 | Gma.13458.1.A1_at | 0.458 | CD398733 | Protein Destination & Storage | Ubiquitin | | FOG: Predicted E3 |
| | | | | | Degradation | | ubiquitin ligase |
| 180 | Gma.16896.1.A1_at | 0.457 | CD416188 | No Homology to | | | |
| | | | | Known Proteins | | | |
| 181 | GmaAffx.15204.1.A1_at | 0.457 | BU549835 | No Homology to | | | |
| | | | | Known Proteins | | | |
| 182 | Gma.412.1.S1_at | 0.456 | BE607835 | Protein Destination & Storage | | | Ubiquitin-protein ligase |
| 183 | Gma.2253.1.S1_at | 0.456 | AF169018.1 | Metabolism | | | Reductases with broad range |
| | | | | | | | of substrate specificities |
| 184 | GmaAffx.51044.1.S1_at | 0.456 | BM522670 | Unclassified - Hypothetical | | | Copine |
| | | | | Protein Supported by | | | |
| | | | | cDNA | | | |
| 185 | Gma.10520.1.A1_at | 0.456 | CD398227 | Unclassified - Hypothetical | | | |
| | | | | Protein Supported by | | | |
| | | | | cDNA | | | |
| 186 | GmaAffx.85565.1.S1_at | 0.456 | BE611082 | Signal Transduction | Calcium Binding | Calcium Dependent | Ca2+/calmodulin-dependent |
| | | | | | | Protein Kinase | protein kinase, EF-Hand protein |
| | | | | | | | superfamily |
| 187 | GmaAffx.78768.1.S1_s_at | 0.456 | CA784898 | No Homology to | | | |
| | | | | Known Proteins | | | |
| 188 | Psojae_rRNA_482_at | 0.455 | | | | | |
| 189 | Gma.14611.1.S1_at | 0.454 | CA852673 | Unclassified - Protein with | | | |
| | | | | Unknown Function | | | |
| 190 | GmaAffx.86745.1.S1_s_at | 0.454 | BI424960 | Disease & Defense | | | |
| 191 | GmaAffx.50617.1.S1_at | 0.454 | AW101420 | Unclassified - Hypothetical | | | |
| | | | | ProteinSupported by | | | |
| | | | | cDNA | | | |
| 192 | Gma.4374.1.S1_at | 0.454 | BM178538 | Metabolism | Cytochrome P450 | CYP76C | Cytochrome P450 CYP2 |
| | | | | | | | subfamily |
| 193 | GmaAffx.93017.1.S1_at | 0.454 | CF808742 | Disease & Defense | | | Soluble epoxide hydrolase |
| 194 | Gma.4673.1.A1_at | 0.453 | BG508468 | No Homology to | | | |
| | | | | Known Proteins | | | |
| 195 | GmaAffx.21773.1.S1_at | 0.453 | BU763882 | Unclassified - Hypothetical | | | |
| | | | | Protein NOT Supported by | | | |
| | | | | cDNA | | | |
| 196 | GmaAffx.24521.1.S1_at | 0.453 | BU761767 | Unclassified - Hypothetical | | | |
| | | | | Protein NOT Supported by | | | |
| | | | | cDNA | | | |
| 197 | Gma.15563.1.S1_at | 0.453 | CD415760 | Unclassified - Hypothetical | | | |
| | | | | Protein Supported by | | | |
| | | | | cDNA | | | |
| 198 | Gma.5513.1.S1_x_at | 0.453 | AW310453 | Metabolism | | | Haloacid dehalogenase-like |
| | | | | | | | hydrolase |
| 199 | GmaAffx.10297.1.S1_at | 0.453 | BE475510 | Metabolism | | | Beta-galactosidase |
| 200 | Gma.3804.1.A1_at | 0.452 | AW310249 | Disease & Defense | | | |
| 201 | Gma.9842.1.S1_at | 0.452 | BM528240 | Unclassified - Hypothetical | | | Leucine rich repeat proteins, |
| | | | | Protein Supported by | | | some proteins contain F-box |
| | | | | cDNA | | | |
| 202 | GmaAffx.18113.2.S1_x_at | 0.452 | AW706795 | Unclassified - Protein with | Hormone Induced | | |
| | | | | Unknown Function | | | |
| 203 | GmaAffx.90853.1.S1_at | 0.452 | CF806578 | No Homology to | | | |
| | | | | Known Proteins | | | |
| 204 | GmaAffx.54778.1.S1_at | 0.451 | BI322039 | No Homology to | | | |
| | | | | Known Proteins | | | |
| 205 | Gma.13042.1.S1_at | 0.45 | CD390874 | Unclassified - Hypothetical | | | |
| | | | | Protein NOT Supported by | | | |
| | | | | cDNA | | | |
| 206 | Gma.6804.1.S1_at | 0.449 | BE657581 | Protein Destination & Storage | | | Aspartyl protease |
| 207 | Gma.17136.1.S1_s_at | 0.449 | BE022719 | Energy | | | Cytochrome b |
| 208 | GmaAffx.5264.1.S1_s_at | 0.448 | BM521512 | Disease & Defense | | | |
| 209 | GmaAffx.92463.1.S1_at | 0.448 | CF808188 | Metabolism | | | NADP+-dependent malic enzyme |
| 210 | GmaAffx.20688.1.S1_at | 0.448 | BG157357 | Unclassified - Protein with | | | Fatty acyl-CoA elongase/ |
| | | | | Unknown Function | | | Polyunsaturated fatty acid |
| | | | | | | | specific elongation enzyme |
| 211 | GmaAffx.81388.1.S1_at | 0.448 | BF424571 | Signal Transduction | Protein Kinase | | Serine/threonine protein kinase |
| 212 | GmaAffx.11892.1.S1_at | 0.447 | BE820605 | Secondary Metabolism | | | Iron/ascorbate family |
| | | | | | | | oxidoreductases |
| 213 | GmaAffx.6017.2.S1_at | 0.446 | BI469662 | Unclassified - Hypothetical | | | |
| | | | | Protein NOT Supported by | | | |
| | | | | cDNA | | | |
| 214 | GmaAffx.91688.1.S1_at | 0.446 | CF807413 | No Homology to | | | |
| | | | | Known Proteins | | | |
| 215 | Gma.3420.1.S1_at | 0.445 | CD406082 | Unclassified - Hypothetical | | | |
| | | | | Protein Supported by | | | |
| | | | | cDNA | | | |
| 216 | GmaAffx.35278.1.S1_at | 0.445 | BE346991 | Disease & Defense | | | |
| 217 | GmaAffx.88786.1.A1_s_at | 0.444 | CK606552 | Cell Structure | | | O-methyltransferase |
| 218 | GmaAffx.73088.1.S1_at | 0.444 | BE475110 | Unclassified - Hypothetical | | | |
| | | | | Protein Supported by | | | |
| | | | | cDNA | | | |
| 219 | Gma.15487.1.S1_at | 0.444 | D63781.1 | Disease & Defense | | | Soluble epoxide hydrolase |
| 220 | Gma.4305.3.S1_a_at | 0.444 | BG044173 | Transporter | Ion Channel Families | Cyclic nucleotide | K+-channel ERG and related |
| | | | | | | gated channel | proteins, contain PAS/PAC sensor |
| | | | | | | family | domain |
| 221 | GmaAffx.77097.1.S1_at | 0.443 | BG237643 | No Homology to | | | |
| | | | | Known Proteins | | | |
| 222 | GmaAffx.18351.1.S1_at | 0.443 | BI472051 | Unclassified - Hypothetical | | | FOG: Armadillo/beta- |
| | | | | Protein Supported by | | | catenin-like repeats |
| | | | | cDNA | | | |
| 223 | Gma.4437.1.S1_s_at | 0.443 | BQ628831 | Secondary Metabolism | Flavonoid | | Flavonol reductase/cinnamoyl-CoA |
| | | | | | Biosynthesis | | reductase |
| 224 | GmaAffx.18599.1.S1_at | 0.443 | BI787464 | Secondary Metabolism | Amines | | Amine oxidase |
| | | | | | Biosynthesis | | |
| 225 | Gma.5418.2.S1_at | 0.442 | BI469627 | Cell Growth | | | |
| | | | | & Division | | | |
| 226 | Gma.5218.1.A1_at | 0.442 | CD392232 | Metabolism | | | Predicted carbonic anhydrase involved in |
| | | | | | | | protection against oxidative damage |
| 227 | GmaAffx.84795.1.S1_at | 0.442 | BG046191 | Unclassified - Hypothetical | | | |
| | | | | Protein NOT Supported by | | | |
| | | | | cDNA | | | |
| 228 | Gma.415.1.A1_at | 0.442 | AW350315 | Protein Destination & Storage | | | FOG: Predicted E3 |
| | | | | | | | ubiquitin ligase |
| 229 | GmaAffx.50980.1.S1_at | 0.442 | BE823095 | Signal Transduction | Protein | | |
| | | | | | Phosphatase | | |
| 230 | Gma.1123.1.S1_at | 0.441 | CD402207 | Unclassified - Hypothetical | | | |
| | | | | Protein Supported by | | | |
| | | | | cDNA | | | |
| 231 | GmaAffx.44484.1.S1_at | 0.441 | BE475213 | Unclassified - Hypothetical | | | |
| | | | | Protein NOT Supported by | | | |
| | | | | cDNA | | | |
| 232 | GmaAffx.26683.1.A1_at | 0.44 | BE820379 | No Homology to | | | |
| | | | | Known Proteins | | | |
| 233 | Gma.17798.2.S1_at | 0.44 | CD412748 | No Homology to | | | |
| | | | | Known Proteins | | | |
| 234 | GmaAffx.93430.1.S1_s_at | 0.439 | CF809155 | Disease & Defense | | | Purple (tartrate-resistant) |
| | | | | | | | acid phosphatase |
| 235 | GmaAffx.34775.1.S1_at | 0.439 | AI794730 | Unclassified - Protein with | | | FOG: Predicted E3 |
| | | | | Unknown Function | | | ubiquitin ligase |
| 236 | GmaAffx.89772.8.S1_x_at | 0.439 | CK605778 | Metabolism | | | |
| 237 | Gma.9259.1.S1_at | 0.439 | CA782161 | Transporter | | | |
| 238 | Gma.5513.1.S1_at | 0.438 | AW310453 | Metabolism | | | Haloacid dehalogenase-like |
| | | | | | | | hydrolase |
| 239 | GmaAffx.17961.2.S1_at | 0.438 | BG511562 | Protein Destination & Storage | | | Molecular chaperones |
| | | | | | | | HSP70/HSC70, HSP70 superfamily |
| 240 | GmaAffx.9482.2.S1_at | 0.438 | AI900615 | Unclassified - Hypothetical | | | Predicted guanosine polyphosphate |
| | | | | Protein NOT Supported by | | | pyrophosphohydrolase/synthase |
| | | | | cDNA | | | |
| 241 | Gma.3589.1.S1_a_at | 0.437 | AW101438 | Secondary Metabolism | GA Biosynthesis | | Iron/ascorbate family |
| | | | | | | | oxidoreductases |
| 242 | Gma.6245.1.S1_at | 0.437 | CA938361 | No Homology to | | | |
| | | | | Known Proteins | | | |
| 243 | Gma.2486.1.S1_at | 0.437 | BF219523 | Unclassified - Hypothetical | | | Iron/ascorbate family |
| | | | | Protein NOT Supported by | | | oxidoreductases |
| | | | | cDNA | | | |
| 244 | Gma.12494.1.S1_at | 0.437 | BG839922 | Protein Destination & Storage | Ubiquitin | E3 Ubiquitin | FOG: Predicted E3 |
| | | | | | Degradation | Ligase | ubiquitin ligase |
| 245 | GmaAffx.51468.1.S1_at | 0.437 | BQ628796 | No Homology to | | | |
| | | | | Known Proteins | | | |
| 246 | Gma.17062.1.S1_at | 0.436 | AW277440 | No Homology to | | | |
| | | | | Known Proteins | | | |
| 247 | GmaAffx.53099.1.S1_at | 0.436 | BG043340 | Unclassified - Hypothetical | | | Alpha-L-fucosidase |
| | | | | Protein NOT Supported by | | | |
| | | | | cDNA | | | |
| 248 | GmaAffx.78163.1.S1_s_at | 0.436 | BM520857 | Signal Transduction | Protein Phosphatase | | Serine/threonine protein |
| | | | | | | | phosphatase |
| 249 | GmaAffx.58073.1.S1_at | 0.436 | CA852105 | Unclassified - Hypothetical | | | |
| | | | | Protein Supported by | | | |
| | | | | cDNA | | | |
| 250 | Gma.892.1.A1_at | 0.435 | BE822836 | Unclassified - Protein with | | | Prolyl 4-hydroxylase alpha |
| | | | | Unknown Function | | | subunit |
| 251 | GmaAffx.74304.1.S1_at | 0.435 | BU577360 | Unclassified - Hypothetical | | | |
| | | | | Protein NOT Supported by | | | |
| | | | | cDNA | | | |
| 252 | GmaAffx.81253.1.S1_at | 0.434 | BE807371 | Unclassified - Protein with | | | |
| | | | | Unknown Function | | | |
| 253 | Gma.18084.1.S1_at | 0.434 | BI317557 | Disease & Defense | | | Ferric reductase, NADH/NADPH oxidase |
| | | | | | | | and related proteins |
| 254 | GmaAffx.91108.1.S1_at | 0.434 | CF806833 | Unclassified - Hypothetical | | | |
| | | | | Protein Supported by | | | |
| | | | | cDNA | | | |
| 255 | Gma.1190.1.S1_at | 0.434 | AF279267.1 | Disease & Defense | | | Acyl-CoA synthetase |
| 256 | GmaAffx.92192.1.S1_s_at | 0.434 | CF806406 | Disease & Defense | | | |
| 257 | Gma.10281.1.S1_at | 0.433 | CA852560 | Unclassified - Protein with | | | Predicted E3 ubiquitin ligase |
| | | | | Unknown Function | | | |
| 258 | Gma.4315.1.S1_at | 0.433 | BM886786 | Unclassified - Protein with | | | |
| | | | | Unknown Function | | | |
| 259 | GmaAffx.71944.1.S1_at | 0.433 | AW351155 | Unclassified - Hypothetical | | | |
| | | | | Protein Supported by | | | |
| | | | | cDNA | | | |
| 260 | GmaAffx.91814.1.S1_at | 0.432 | CF807539 | Unclassified - Hypothetical | | | |
| | | | | Protein Supported by | | | |
| | | | | cDNA | | | |
| 261 | GmaAffx.84751.1.A1_at | 0.432 | AW349702 | Transcription | Transcription | MYB | Transcription factor, Myb |
| | | | | | Factor | | superfamily |
| 262 | Gma.15773.1.A1_at | 0.431 | CD406280 | Metabolism | | | |
| 263 | Gma.12873.1.S1_at | 0.431 | CD393774 | No Homology to | | | |
| | | | | Known Proteins | | | |
| 264 | GmaAffx.9926.2.S1_at | 0.431 | BF597587 | Unclassified - Hypothetical | | | |
| | | | | Protein Supported by | | | |
| | | | | cDNA | | | |
| 265 | Gma.14312.1.S1_at | 0.431 | CA852635 | No Homology to | | | |
| | | | | Known Proteins | | | |
| 266 | Gma.15470.1.S1_at | 0.43 | BU546303 | Unclassified - Hypothetical | | | |
| | | | | Protein Supported by | | | |
| | | | | cDNA | | | |
| 267 | GmaAffx.15222.1.S1_at | 0.43 | BU550700 | Unclassified - Hypothetical | | | |
| | | | | Protein NOT Supported by | | | |
| | | | | cDNA | | | |
| 268 | GmaAffx.4505.1.S1_at | 0.43 | AW306682 | Unclassified - Protein with | | | Predicted NUDIX hydrolase |
| | | | | Unknown Function | | | FGF-2 and related proteins |
| 268 | GmaAffx.4505.1.S1_at | 0.43 | AW306682 | Unclassified - Protein with | | | Cdc4 and related F-box and |
| | | | | Unknown Function | | | WD-40 proteins |
| 269 | GmaAffx.90827.1.S1_at | 0.43 | CF806552 | Unclassified - Hypothetical | | | Molecular chaperone |
| | | | | Protein Supported by | | | (small heat-shock protein Hsp26/Hsp42) |
| | | | | cDNA | | | |
| 270 | GmaAffx.92184.1.S1_s_at | 0.43 | CF807909 | Unclassified - Protein with | | | |
| | | | | Unknown Function | | | |
| 271 | GmaAffx.93592.1.S1_x_at | 0.429 | CF809317 | Unclassified - Hypothetical | | | |
| | | | | Protein Supported by | | | |
| | | | | cDNA | | | |
| 272 | GmaAffx.89971.1.S1_s_at | 0.429 | CF805696 | Intracellular Traffic | | | Annexin |
| 273 | GmaAffx.66982.1.S1_at | 0.428 | BI970847 | Protein Destination & Storage | | | Molecular chaperone |
| | | | | | | | (small heat-shock protein Hsp26/Hsp42) |
| 274 | GmaAffx.45236.1.S1_at | 0.428 | BG156638 | Disease & Defense | | | Purple |
| | | | | | | | (tartrate-resistant) |
| | | | | | | | acid phosphatase |
| 275 | GmaAffx.79888.1.S1_at | 0.428 | AI988123 | Signal Transduction | Protein Kinase | | |
| 276 | Gma.6075.1.S1_at | 0.428 | AW317183 | Disease & Defense | | | |
| 277 | Gma.10795.1.S1_at | 0.428 | AI442845 | Cell Structure | | | |
| 278 | GmaAffx.55244.1.A1_at | 0.427 | BU549116 | No Homology to | | | |
| | | | | Known Proteins | | | |
| 279 | GmaAffx.69792.1.S1_s_at | 0.427 | BU550596 | Transcription | Transcription | AP2/EREBP | |
| | | | | | Factor | | |
| 280 | GmaAffx.30428.1.S1_at | 0.427 | BE824410 | Protein Destination & Storage | | | Molecular chaperones |
| | | | | | | | HSP70/HSC70, HSP70 superfamily |
| 281 | GmaAffx.90104.1.S1_s_at | 0.427 | CF805829 | Transporter | | | Permease of the |
| | | | | | | | major facilitator |
| | | | | | | | superfamily |
| 282 | Gma.3969.2.S1_at | 0.426 | CD412522 | Energy | | | |
| 283 | Gma.16547.1.S1_at | 0.426 | BU545050 | Transcription | Transcription | WRKY | |
| | | | | | Factor | | |
| 284 | GmaAffx.74211.1.S1_at | 0.426 | BE820853 | Protein Destination & Storage | | | Aspartyl protease |
| 285 | GmaAffx.42667.3.S1_at | 0.426 | AW306785 | Transcription | Transcription | GRAS | |
| | | | | | Factor | | |
| 286 | Gma.2772.1.S1_at | 0.426 | BG238134 | No Homology to | | | |
| | | | | Known Proteins | | | |
| 287 | Gma.5889.1.S1_at | 0.425 | BQ610202 | Unclassified - Hypothetical | | | Endoplasmic reticulum protein |
| | | | | Protein NOT Supported by | | | EP58, contains filamin rod |
| | | | | cDNA | | | domain and KDEL motif |
| 288 | GmaAffx.18742.1.S1_at | 0.425 | BI970450 | Unclassified - Hypothetical | | | Predicted membrane protein |
| | | | | Protein Supported by | | | |
| | | | | cDNA | | | |
| 289 | Gma.1947.1.S1_at | 0.425 | BI968172 | Transcription | Transcription | Homeo domain | Transcription factor MEIS1 |
| | | | | | Factor | | and related HOX domain proteins |
| 290 | Gma.5400.1.S1_at | 0.425 | BM093775 | No Homology to | | | |
| | | | | Known Proteins | | | |
| 291 | Gma.6709.1.S1_at | 0.424 | BE823291 | Signal Transduction | Protein Kinase | AtCIPKs | Serine/threonine protein kinase |
| 292 | Gma.11131.1.S1_at | 0.424 | BI968388 | Protein Destination & Storage | | | Molecular chaperone |
| | | | | | | | (small heat-shock protein Hsp26/Hsp42) |
| 293 | GmaAffx.72580.1.S1_at | 0.424 | BG157182 | Unclassified - Hypothetical | | | |
| | | | | Protein Supported by | | | |
| | | | | cDNA | | | |
| 294 | Gma.4432.1.S1_a_at | 0.423 | BQ610841 | Unclassified - Protein with | Hormone Induced | | Voltage-gated shaker-like K+ |
| | | | | Unknown Function | | | channel, subunit |
| | | | | | | | beta/KCNAB |
| 295 | GmaAffx.74194.1.S1_at | 0.423 | BI968058 | Metabolism | | | 17 beta-hydroxy-steroid |
| | | | | | | | dehydrogenase type 3, HSD17B3 |
| 296 | GmaAffx.62375.1.S1_at | 0.423 | BQ612727 | Unclassified - Hypothetical | | | |
| | | | | Protein Supported by | | | |
| | | | | cDNA | | | |
| 297 | GmaAffx.38733.1.A1_at | 0.422 | BE657590 | No Homology to | | | |
| | | | | Known Proteins | | | |
| 298 | GmaAffx.65580.1.S1_at | 0.422 | CA799171 | Metabolism | | | |
| 299 | GmaAffx.81618.1.S1_at | 0.421 | AW706403 | No Homology to | | | |
| | | | | Known Proteins | | | |
| 300 | GmaAffx.16201.1.S1_at | 0.421 | BU550725 | No Homology to | | | |
| | | | | Known Proteins | | | |
| 301 | GmaAffx.55124.1.S1_at | 0.421 | BI970821 | Unclassified - Hypothetical | | | Metacaspase involved in |
| | | | | Protein Supported by | | | regulation of apoptosis |
| | | | | cDNA | | | |
| 302 | Gma.4755.1.S1_at | 0.421 | AW310232 | Unclassified - Hypothetical | | | |
| | | | | Protein NOT Supported by | | | |
| | | | | cDNA | | | |
| 303 | GmaAffx.9059.1.S1_at | 0.421 | AW423327 | No Homology to | | | |
| | | | | Known Proteins | | | |
| 304 | GmaAffx.51614.1.S1_at | 0.42 | BU089837 | No Homology to | | | |
| | | | | Known Proteins | | | |
| 305 | Gma.153.1.S1_x_at | 0.42 | Y10491.1 | Metabolism | Cytochrome P450 | CYP82C | Cytochrome P450 CYP2 |
| | | | | | | | subfamily |
| 306 | GmaAffx.50980.1.S1_x_at | 0.419 | BE823095 | Signal Transduction | Protein Phosphatase | | |
| 307 | GmaAffx.47716.1.A1_at | 0.418 | CD397037 | Unclassified - Hypothetical | | | |
| | | | | Protein NOT Supported by | | | |
| | | | | cDNA | | | |
| 308 | Gma.4680.1.A1_s_at | 0.418 | AW309707 | Unclassified - Protein with | | | |
| | | | | Unknown Function | | | |
| 309 | Gma.17288.1.A1_at | 0.418 | AW310692 | No Homology to | | | |
| | | | | Known Proteins | | | |
| 310 | Gma.5258.1.S1_at | 0.418 | BE658899 | No Homology to | | | |
| | | | | Known Proteins | | | |
| 311 | GmaAffx.2710.1.S1_at | 0.417 | BE822538 | Unclassified - Hypothetical | | | Endoplasmic reticulum protein |
| | | | | Protein Supported by | | | EP58, contains filamin rod domain |
| | | | | cDNA | | | and KDEL motif |
| 312 | GmaAffx.89049.1.S1_at | 0.417 | CK606168 | Intracellular Traffic | | | Annexin |
| 313 | Gma.6046.1.S1_at | 0.417 | BE022758 | Unclassified - Hypothetical | | | |
| | | | | Protein NOT Supported by | | | |
| | | | | cDNA | | | |
| 314 | Gma.4839.1.A1_at | 0.417 | AW310019 | Metabolism | | | Branched chain aminotransferase |
| | | | | | | | BCAT1, pyridoxal phosphate enzymes |
| | | | | | | | type IV superfamily |
| 315 | GmaAffx.26320.1.S1_at | 0.416 | BE440416 | Unclassified - Hypothetical | | | |
| | | | | Protein Supported by | | | |
| | | | | cDNA | | | |
| 316 | Gma.10969.1.S1_x_at | 0.416 | U36191.1 | Metabolism | | | |
| 317 | GmaAffx.90234.1.S1_at | 0.416 | CF805959 | Transcription | Transcription | Zinc Finger | FOG: Zn-finger |
| | | | | | Factor | | |
| 318 | GmaAffx.5232.1.S1_at | 0.416 | BM309521 | Unclassified - Protein with | | | |
| | | | | Unknown Function | | | |
| 319 | GmaAffx.28578.1.S1_at | 0.416 | BQ094224 | Disease & Defense | | | |
| 320 | Gma.1326.1.S1_at | 0.416 | CD397515 | Secondary Metabolism | | | |
| 321 | GmaAffx.59762.1.S1_at | 0.416 | BI971811 | Unclassified - Protein with | | | |
| | | | | Unknown Function | | | |
| 322 | Gma.1715.1.S1_at | 0.415 | BE191618 | Metabolism | | | |
| 323 | Gma.18020.1.S1_at | 0.415 | AW309711 | Signal Transduction | Protein Kinase | | Serine/threonine protein kinase |
| 324 | GmaAffx.10580.1.S1_at | 0.414 | BF424010 | Transporter | | | Amino acid |
| | | | | | | | transporters |
| 325 | GmaAffx.12369.1.A1_at | 0.414 | CD412820 | Unclassified - Hypothetical | | | |
| | | | | Protein Supported by | | | |
| | | | | cDNA | | | |
| 326 | Gma.5256.2.S1_at | 0.414 | AI441316 | Unclassified - Hypothetical | | | |
| | | | | Protein NOT Supported by | | | |
| | | | | cDNA | | | |
| 327 | GmaAffx.1890.1.S1_s_at | 0.414 | BI970941 | Transcription | Transcription | Heat Shock | Heat shock transcription factor |
| | | | | | Factor | | |
| 328 | Gma.10266.1.S1_at | 0.414 | BG157207 | No Homology to | | | |
| | | | | Known Proteins | | | |
| 329 | Gma.17535.1.S1_at | 0.413 | BM178659 | Unclassified - Hypothetical | | | Uncharacterized conserved protein, |
| | | | | Protein Supported by | | | contains double-stranded beta-helix |
| | | | | cDNA | | | domain |
| 330 | Gma.4359.1.S1_at | 0.413 | AI794712 | Energy | | | Thioredoxin |
| 331 | Gma.7836.2.S1_at | 0.413 | BI425718 | Unclassified - Hypothetical | | | |
| | | | | Protein NOT Supported by | | | |
| | | | | cDNA | | | |
| 332 | Gma.17502.2.S1_at | 0.413 | BG046201 | Transcription | Transcription | Unclassified | Zuotin and related molecular |
| | | | | | Factor | | chaperones (DnaJ superfamily), |
| | | | | | | | contains DNA-binding domains |
| 333 | Gma.4839.2.S1_at | 0.412 | BI974385 | Metabolism | | | Branched chain |
| | | | | | | | aminotransferase |
| | | | | | | | BCAT1, pyridoxal |
| | | | | | | | phosphate enzymes |
| | | | | | | | type IV superfamily |
| 334 | Gma.5162.1.A1_at | 0.412 | BI971156 | Signal Transduction | Protein Kinase | | |
| 335 | Gma.11195.1.S1_at | 0.412 | CD417406 | Transporter | | | Apolipoprotein D/Lipocalin |
| 336 | Gma.11028.1.S1_at | 0.412 | BE823084 | Metabolism | | | Glyoxylate/hydroxypyruvate |
| | | | | | | | reductase (D-isomer-specific |
| | | | | | | | 2-hydroxy acid dehydrogenase superfamily) |
| 337 | Gma.16521.1.S1_at | 0.412 | BE609300 | Unclassified - Hypothetical | | | Uncharacterized conserved protein |
| | | | | Protein Supported by | | | |
| | | | | cDNA | | | |
| 338 | Gma.7824.1.S1_at | 0.412 | BU550492 | Metabolism | | | Kynurenine 3-monooxygenase |
| | | | | | | | and related flavoprotein monooxygenases |
| 339 | GmaAffx.76346.1.A1_at | 0.412 | BQ612378 | Energy | | | 1,4-benzoquinone reductase-like; |
| | | | | | | | Trp repressor binding protein-like/ |
| | | | | | | | protoplast-secreted protein |
| 340 | GmaAffx.91108.1.S1_s_at | 0.411 | CF806833 | Unclassified - Hypothetical | | | |
| | | | | ProteinSupported by | | | |
| | | | | cDNA | | | |
| 341 | Gma.5610.1.S1_at | 0.41 | AW348190 | Unclassified - Hypothetical | | | |
| | | | | Protein Supported by | | | |
| | | | | cDNA | | | |
| 342 | GmaAffx.60673.1.S1_at | 0.409 | BU080770 | Unclassified - Hypothetical | | | |
| | | | | Protein NOT Supported by | | | |
| | | | | cDNA | | | |
| 343 | Gma.16477.1.S1_at | 0.409 | BE824134 | No Homology to | | | |
| | | | | Known Proteins | | | |
| 344 | Gma.4455.2.S1_at | 0.409 | BE059560 | Signal Transduction | Protein Kinase | | Serine/threonine protein kinase |
| 345 | Gma.10581.1.S1_at | 0.409 | AW396439 | Unclassified - Hypothetical | | | Predicted haloacid |
| | | | | Protein Supported by | | | dehalogenase-like hydrolase |
| | | | | cDNA | | | |
| 346 | GmaAffx.26299.1.S1_at | 0.408 | BQ299016 | Signal Transduction | Protein Kinase | | |
| 347 | Gma.13241.1.S1_at | 0.408 | CD395739 | Unclassified - Hypothetical | | | |
| | | | | Protein NOT Supported by | | | |
| | | | | cDNA | | | |
| 348 | Gma.7120.2.S1_a_at | 0.408 | BG041285 | Metabolism | | | Alanine-glyoxylate |
| | | | | | | | aminotransferase AGT2 |
| 349 | Gma.7227.1.S1_at | 0.408 | BU551112 | Protein Destination & Storage | | | Ubiquitin-protein ligase |
| 350 | GmaAffx.57970.1.S1_at | 0.407 | BM886862 | Transcription | Transcription | NAC Domain | |
| | | | | | Factor | | |
| 351 | Gma.17806.1.S1_at | 0.406 | BE820775 | Transporter | | | H+/oligopeptide symporter |
| 352 | Gma.14295.1.S1_at | 0.405 | CA852253 | No Homology to | | | |
| | | | | Known Proteins | | | |
| 353 | Gma.6761.1.A1_at | 0.405 | BQ453407 | Cell Structure | | | Beta-glucosidase, lactase |
| | | | | | | | phlorizinhydrolase, and related proteins |
| 354 | GmaAffx.26859.1.S1_s_at | 0.405 | BM085620 | Disease & Defense | | | Glutathione S-transferase |
| 355 | GmaAffx.83564.1.S1_at | 0.405 | BU578309 | No Homology to | | | |
| | | | | Known Proteins | | | |
| 356 | Gma.17642.1.S1_at | 0.405 | BU550265 | Unclassified - Hypothetical | | | BPI/LBP/CETP family protein |
| | | | | Protein Supported by | | | |
| | | | | cDNA | | | |
| 357 | GmaAffx.73303.1.S1_at | 0.405 | BE190156 | Unclassified - Hypothetical | | | Predicted membrane protein |
| | | | | Protein Supported by | | | |
| | | | | cDNA | | | |
| 358 | Gma.3283.1.S1_at | 0.404 | BQ298733 | No Homology to | | | |
| | | | | Known Proteins | | | |
| 359 | Gma.7487.2.A1_at | 0.403 | AW349599 | Transporter | | | Amino acid transporters |
| 360 | GmaAffx.7738.1.S1_s_at | 0.403 | BE058056 | No Homology to | | | |
| | | | | Known Proteins | | | |
| 361 | Gma.4124.1.S1_at | 0.403 | BI787263 | Transcription | Transcription | Homeodomain | |
| | | | | | Factor | | |
| 362 | GmaAffx.89141.1.S1_at | 0.403 | CK605765 | Cell Structure | | | O-methyltransferase |
| 363 | Gma.7051.1.A1_at | 0.403 | BU544101 | Disease & Defense | | | Metacaspase involved in |
| | | | | | | | regulation of apoptosis |
| 364 | Gma.10854.1.S1_at | 0.403 | CA802334 | No Homology to | | | |
| | | | | Known Proteins | | | |
| 365 | GmaAffx.81502.1.S1_at | 0.403 | CD404946 | Unclassified - Hypothetical | | | Uncharacterized conserved protein |
| | | | | Protein NOT Supported by | | | |
| | | | | cDNA | | | |
| 366 | GmaAffx.53108.1.S1_at | 0.403 | BM093748 | SignalTransduction | Protein Kinase | | Serine/threonine protein |
| | | | | | | | kinase |
| 367 | Gma.15469.1.S1_at | 0.402 | BU546655 | Cell Structure | | | Alpha tubulin |
| 368 | GmaAffx.24809.1.S1_at | 0.402 | CA820582 | Unclassified - Hypothetical | | | |
| | | | | Protein NOT Supported by | | | |
| | | | | cDNA | | | |
| 369 | Gma.1114.1.S1_s_at | 0.402 | CD403139 | Unclassified - Hypothetical | | | |
| | | | | Protein NOT Supported by | | | |
| | | | | cDNA | | | |
| 370 | GmaAffx.92230.1.S1_at | 0.402 | CF807302 | Disease & Defense | | | |
| 371 | GmaAffx.10590.1.S1_s_at | 0.402 | BU551319 | Signal Transduction | Protein Kinase | | |
| 372 | GmaAffx.63822.1.S1_at | 0.402 | BM520838 | Disease & Defense | | | Purple (tartrate-resistant) |
| | | | | | | | acid phosphatase |
| 373 | GmaAffx.84607.2.S1_at | 0.401 | BU080867 | Unclassified - Hypothetical | | | |
| | | | | Protein Supported by | | | |
| | | | | cDNA | | | |
| 374 | GmaAffx.54085.1.S1_at | 0.401 | BI970762 | Unclassified - Hypothetical | | | |
| | | | | Protein Supported by | | | |
| | | | | cDNA | | | |
| 375 | GmaAffx.50347.1.S1_at | 0.4 | BQ740576 | Signal Transduction | Protein Kinase | | |
| 376 | GmaAffx.1694.1.S1_at | 0.4 | BQ454286 | Transcription | Transcription | MYB | |
| | | | | | Factor | | |
| 377 | Gma.1980.1.A1_at | 0.4 | BM520466 | Signal Transduction | Protein Kinase | AtCIPKs | Serine/threonine protein kinase |
| 378 | GmaAffx.60479.4.S1_at | 0.4 | BG507369 | Transcription | Transcription | Homeodomain | Transcription factor MEIS1 |
| | | | | | Factor | | and related HOX domain proteins |
| 379 | GmaAffx.15983.2.S1_at | 0.399 | AW279540 | Signal Transduction | Protein Phosphatase | | Serine/threonine protein phosphatase |
| 380 | GmaAffx.6824.2.S1_at | 0.399 | BQ094655 | Cell Structure | | | |
| 381 | Gma.3589.1.S1_at | 0.399 | AW101438 | Secondary Metabolism | GA Biosynthesis | | Iron/ascorbate family |
| | | | | | | | oxidoreductases |
| 382 | Gma.16585.1.S1_x_at | 0.399 | CA850597 | Metabolism | | | Predicted carbonic anhydrase |
| | | | | | | | involved in protection against |
| | | | | | | | oxidative damage |
| 383 | GmaAffx.57967.1.S1_at | 0.399 | AW102170 | Protein Destination & Storage | | | |
| 384 | Gma.5701.2.S1_at | 0.398 | BI701059 | Unclassified - Hypothetical | | | |
| | | | | Protein Supported by | | | |
| | | | | cDNA | | | |
| 385 | Gma.10885.1.S1_at | 0.398 | BQ610245 | Unclassified - Hypothetical | | | Predicted kinase |
| | | | | Protein Supported by | | | |
| | | | | cDNA | | | |
| 386 | Gma.11665.2.A1_at | 0.397 | BU545959 | No Homology to | | | |
| | | | | Known Proteins | | | |
| 387 | Gma.10626.3.S1_at | 0.397 | BE022458 | Secondary Metabolism | | | Iron/ascorbate family |
| | | | | | | | oxidoreductases |
| 388 | Gma.5038.1.A1_at | 0.397 | BE023631 | Disease & Defense | | | Acyl-CoA synthetase |
| 389 | GmaAffx.43786.2.S1_at | 0.396 | BU080930 | Signal Transduction | Histidine Kinase | | Sensory transduction histidine kinase |
| 390 | GmaAffx.24844.1.S1_at | 0.396 | BU765373 | Unclassified - Hypothetical | | | Uncharacterized conserved protein |
| | | | | Protein Supported by | | | |
| | | | | cDNA | | | |
| 391 | GmaAffx.27787.1.A1_at | 0.396 | BG789758 | Transporter | | | Transporter, ABC superfamily |
| | | | | | | | (Breast cancer resistance protein) |
| 392 | Gma.1380.1.S1_at | 0.396 | CA935076 | Metabolism | | | |
| 393 | Gma.4260.1.S1_at | 0.396 | BM177256 | Unclassified - Hypothetical | | | |
| | | | | Protein NOT Supported by | | | |
| | | | | cDNA | | | |
| 394 | GmaAffx.74693.1.S1_at | 0.396 | CD406171 | Metabolism | | | |
| 395 | Gma.5709.1.S1_at | 0.395 | BG157556 | Unclassified - Protein with | | | |
| | | | | Unknown Function | | | |
| 396 | GmaAffx.22297.2.S1_at | 0.395 | BM271210 | Transcription | Transcription | WRKY | |
| | | | | | Factor | | |
| 397 | Gma.5578.2.S1_a_at | 0.394 | AW310579 | Metabolism | Cytochrome P450 | CYP98A | Cytochrome P450 CYP2 |
| | | | | | | | subfamily |
| 398 | GmaAffx.74160.1.S1_at | 0.394 | BQ740845 | Protein Destination & Storage | | | |
| 399 | Gma.177.1.S1_at | 0.393 | AF004806.1 | Protein Destination & Storage | Storage Protein | | Reticulon |
| 400 | GmaAffx.13730.1.S1_at | 0.393 | BI316755 | Transcription | Transcription | Basic Helix- | |
| | | | | | Factor | Loop- Helix | |
| | | | | | | (bHLH) | |
| 401 | GmaAffx.28870.1.S1_at | 0.393 | BG726532 | Unclassified - Hypothetical | | | |
| | | | | Protein Supported by | | | |
| | | | | cDNA | | | |
| 402 | GmaAffx.73195.1.S1_at | 0.393 | BI701045 | Secondary Metabolism | Flavonoid | | Flavonol reductase/cinnamoyl-CoA |
| | | | | | Biosynthesis | | reductase |
| 403 | Gma.16147.1.S1_at | 0.393 | BE473577 | Transcription | Transcription | Basic Helix- | |
| | | | | | Factor | Loop-Helix | |
| | | | | | | (bHLH) | |
| 404 | GmaAffx.23789.1.S1_at | 0.392 | BU551014 | Unclassified - Hypothetical | | | |
| | | | | Protein Supported by | | | |
| | | | | cDNA | | | |
| 405 | GmaAffx.37636.1.S1_at | 0.392 | BQ610347 | Metabolism | Amino Acid Biosynthesis/ | | Alanine-glyoxylate |
| | | | | | Metabolism | | aminotransferase AGT2 |
| 406 | Gma.7047.1.S1_s_at | 0.392 | CD417401 | Unclassified - Protein with | | | |
| | | | | Unknown Function | | | |
| 407 | Gma.1649.1.A1_at | 0.392 | BE659535 | No Homology to | | | |
| | | | | Known Proteins | | | |
| 408 | GmaAffx.8304.1.A1_at | 0.391 | BE822695 | Unclassified - Hypothetical | | | Possible oxidoreductase |
| | | | | Protein Supported by | | | |
| | | | | cDNA | | | |
| 409 | GmaAffx.3726.1.A1_at | 0.391 | BE822942 | Unclassified - Hypothetical | | | |
| | | | | Protein Supported by | | | |
| | | | | cDNA | | | |
| 410 | Gma.5215.1.S1_at | 0.391 | AI988141 | Unclassified - Hypothetical | | | BCL2-associated athanogene-like proteins |
| | | | | Protein Supported by | | | and related BAG family chaperone |
| | | | | cDNA | | | regulators |
| 411 | Gma.17605.1.S1_at | 0.39 | BI968208 | Secondary | Flavonoid | | |
| | | | | Metabolism | Biosynthesis | | |
| 412 | GmaAffx.58125.1.A1_at | 0.39 | BE347561 | Transcription | Transcription | Basic Helix- | |
| | | | | | Factor | Loop-Helix | |
| | | | | | | (bHLH) | |
| 413 | GmaAffx.89624.1.A1_s_at | 0.39 | CK606100 | Transcription | Transcription | Basic | |
| | | | | | Factor | Leucine | |
| | | | | | | Zipper | |
| | | | | | | (bZIP) | |
| 414 | Gma.3745.1.S1_at | 0.389 | AW156339 | Secondary Metabolism | Amines Biosynthesis | | Amine oxidase |
| 415 | GmaAffx.82034.1.S1_at | 0.389 | BM887093 | Transcription | Transcription | Homeodomain | Transcription factor HEX, contains |
| | | | | | Factor | | HOX and HALZ domains |
| 416 | Gma.16336.1.S1_at | 0.389 | BE659555 | Protein Destination & Storage | | | Predicted pilin-like |
| | | | | | | | transcription factor |
| 417 | Gma.16276.2.S1_a_at | 0.388 | BG316414 | Unclassified - Hypothetical | | | FOG: PPR repeat |
| | | | | Protein Supported by | | | |
| | | | | cDNA | | | |
| 418 | Gma.13146.1.S1_at | 0.388 | BM731716 | Unclassified - Hypothetical | | | |
| | | | | Protein Supported by | | | |
| | | | | cDNA | | | |
| 419 | GmaAffx.86020.1.S1_at | 0.388 | BI701699 | Signal Transduction | Protein Kinase | AtCIPKs | Serine/threonine protein kinase |
| 420 | Gma.3591.1.S1_a_at | 0.388 | BE440266 | Secondary Metabolism | Ethylene | | Iron/ascorbate family |
| | | | | | Biosynthesis | | oxidoreductases |
| 421 | GmaAffx.68853.1.S1_at | 0.388 | CA803108 | Unclassified - Hypothetical | | | |
| | | | | Protein Supported by | | | |
| | | | | cDNA | | | |
| 422 | GmaAffx.73021.1.S1_at | 0.387 | BE556340 | Disease & Defense | | | |
| 423 | GmaAffx.80899.1.S1_at | 0.387 | AW348263 | Unclassified - Hypothetical | | | |
| | | | | Protein Supported by | | | |
| | | | | cDNA | | | |
| 424 | GmaAffx.77052.1.S1_at | 0.387 | BE556140 | Transporter | Inorganic Solute | Putative potassium | |
| | | | | | Cotransporters | transporter family | |
| 425 | GmaAffx.91731.1.S1_s_at | 0.387 | CF807456 | Secondary Metabolism | Ethylene | | Iron/ascorbate family |
| | | | | | Biosynthesis | | oxidoreductases |
| 426 | GmaAffx.77052.2.A1_at | 0.387 | BG155285 | Transporter | Inorganic Solute | Putative potassium | |
| | | | | | Cotransporters | transporter | |
| | | | | | | family | |
| 427 | GmaAffx.17924.1.S1_at | 0.387 | BG157338 | Unclassified - Protein with | | | |
| | | | | Unknown Function | | | |
| 428 | GmaAffx.59014.1.S1_s_at | 0.386 | AW733977 | No Homology to | | | |
| | | | | Known Proteins | | | |
| 429 | Gma.8946.1.S1_at | 0.386 | BQ453813 | Transcription | Transcription | NAC Domain | |
| | | | | | Factor | | |
| 430 | Gma.2475.1.S1_at | 0.384 | BM522941 | Cell Structure | | | |
| 431 | Gma.6730.1.S1_at | 0.384 | BE211440 | Metabolism | | | UDP-glucuronosyl and UDP- |
| | | | | | | | glucosyl transferase |
| 432 | GmaAffx.13912.1.S1_at | 0.384 | BI699258 | Disease & Defense | | | Purple (tartrate-resistant) |
| | | | | | | | acid phosphatase |
| 433 | Gma.5038.2.S1_a_at | 0.384 | CD417606 | Disease & Defense | | | Acyl-CoA synthetase |
| 434 | Gma.12817.1.A1_at | 0.384 | CD405335 | Unclassified - Hypothetical | | | |
| | | | | Protein Supported by | | | |
| | | | | cDNA | | | |
| 435 | GmaAffx.31571.1.S1_at | 0.384 | BU551140 | Unclassified - Hypothetical | | | |
| | | | | Protein NOT Supported by | | | |
| | | | | cDNA | | | |
| 436 | Gma.4990.1.A1_at | 0.383 | BI315863 | Unclassified - Hypothetical | | | Uncharacterized conserved protein |
| | | | | Protein Supported by | | | |
| | | | | cDNA | | | |
| 437 | Gma.13340.1.S1_at | 0.382 | BM523855 | Metabolism | | | UDP-glucuronosyl and UDP- |
| | | | | | | | glucosyl transferase |
| 438 | GmaAffx.28196.2.A1_s_at | 0.382 | CD416781 | Metabolism | | | Long-chain acyl-CoA |
| | | | | | | | synthetases (AMP-forming) |
| 439 | GmaAffx.90585.1.S1_s_at | 0.382 | CF806310 | Protein Destination & Storage | | | Aspartyl protease |
| 440 | GmaAffx.62856.1.S1_at | 0.382 | AW432978 | Signal Transduction | MAPKKK | ZIK | Serine/threonine protein kinase |
| 441 | GmaAffx.6.1.S1_at | 0.381 | AW348562 | No Homology to | | | |
| | | | | Known Proteins | | | |
| 442 | Gma.2565.1.S1_at | 0.381 | BG652533 | Unclassified - Hypothetical | | | |
| | | | | Protein Supported by | | | |
| | | | | cDNA | | | |
| 443 | Gma.8706.1.S1_at | 0.38 | BU762562 | Unclassified - Hypothetical | | | |
| | | | | Protein Supported by | | | |
| | | | | cDNA | | | |
| 444 | GmaAffx.39467.1.S1_at | 0.38 | BQ299569 | Unclassified - Hypothetical | | | |
| | | | | Protein NOT Supported by | | | |
| | | | | cDNA | | | |
| 445 | GmaAffx.90789.1.S1_s_at | 0.38 | CF806514 | Unclassified - Protein with | | | |
| | | | | Unknown Function | | | |
| 446 | Gma.9828.1.S1_at | 0.38 | AF529301.1 | Metabolism | | | Haloacid dehalogenase-like hydrolase |
| 447 | GmaAffx.93642.1.S1_s_at | 0.379 | CF809367 | Disease & Defense | | | |
| 448 | GmaAffx.6790.1.S1_at | 0.379 | AI856683 | Disease & Defense | | | Soluble epoxide hydrolase |
| 449 | Gma.4049.1.S1_at | 0.379 | BQ786519 | Signal Transduction | Protein Kinase | | Serine/threonine protein kinase |
| 450 | GmaAffx.38961.1.S1_at | 0.379 | BU081740 | Transcription | Transcription | MYB | Transcription factor, Myb |
| | | | | | Factor | | superfamily |
| 451 | Gma.5691.1.S1_at | 0.379 | BM522643 | No Homology to | | | |
| | | | | Known Proteins | | | |
| 452 | Gma.5578.3.S1_at | 0.379 | AW310572 | Metabolism | Cytochrome P450 | CYP98A | Cytochrome P450 CYP2 |
| | | | | | | | subfamily |
| 453 | GmaAffx.85878.1.S1_at | 0.378 | BF067810 | Intracellular Traffic | | | Aquaporin |
| | | | | | | | (major intrinsic protein family) |
| 454 | GmaAffx.42856.1.S1_at | 0.378 | BM567981 | Transcription | Transcription | Basic Helix- | |
| | | | | | Factor | Loop-Helix | |
| | | | | | | (bHLH) | |
| 455 | Gma.6343.1.S1_at | 0.377 | AI442963 | Unclassified - Hypothetical | | | |
| | | | | Protein NOT Supported by | | | |
| | | | | cDNA | | | |
| 456 | GmaAffx.61571.2.S1_at | 0.377 | AI939190 | Unclassified - Hypothetical | | | |
| | | | | Protein NOT Supported by | | | |
| | | | | cDNA | | | |
| 457 | GmaAffx.59808.1.S1_at | 0.377 | BM886228 | Transporter | | | Predicted transporter |
| | | | | | | | (major facilitator superfamily) |
| 458 | Gma.2831.1.S1_s_at | 0.376 | AW349413 | Transcription | Transcription | Zinc Finger | |
| | | | | | Factor | | |
| 459 | GmaAffx.40923.1.S1_at | 0.376 | BG653848 | Disease & Defense | | | Selenium-binding protein |
| 460 | Gma.559.1.A1_at | 0.376 | BQ630327 | Protein Destination & Storage | | | |
| 461 | GmaAffx.24451.1.A1_at | 0.375 | BU550405 | Metabolism | Cytochrome P450 | CYP90C | Cytochrome P450 |
| | | | | | | | CYP4/CYP19/CYP26 subfamilies |
| 462 | GmaAffx.38558.1.S1_at | 0.375 | CD407094 | Metabolism | | | NADP+-dependent |
| | | | | | | | malic enzyme |
| 463 | GmaAffx.17892.2.S1_at | 0.375 | BQ628677 | No Homology to | | | |
| | | | | Known Proteins | | | |
| 464 | GmaAffx.2745.2.A1_at | 0.374 | BQ611985 | Unclassified - Hypothetical | | | Acetylglucosaminyltransferase |
| | | | | Protein Supported by | | | EXT1/exostosin I |
| | | | | cDNA | | | |
| 465 | GmaAffx.91254.1.S1_at | 0.374 | CF806979 | Unclassified - Hypothetical | | | |
| | | | | Protein Supported by | | | |
| | | | | cDNA | | | |
| 466 | GmaAffx.46770.1.S1_at | 0.374 | BQ298902 | Transporter | Ion Channel | Putative ligand- | Glutamate-gated kainate-type |
| | | | | | Families | gated ion channel | ion channel receptor subunit |
| | | | | | | subunit family | GluR5 and related subunits |
| 467 | GmaAffx.1620.1.S1_at | 0.374 | AW424105 | Unclassified - Hypothetical | | | |
| | | | | Protein NOT Supported by | | | |
| | | | | cDNA | | | |
| 468 | GmaAffx.90570.1.S1_at | 0.374 | CF806295 | Unclassified - Hypothetical | | | |
| | | | | Protein Supported by | | | |
| | | | | cDNA | | | |
| 469 | GmaAffx.84964.1.S1_at | 0.373 | AW598267 | No Homology to | | | |
| | | | | Known Proteins | | | |
| 470 | GmaAffx.64644.1.S1_at | 0.373 | AW348952 | Signal Transduction | Protein Kinase | | Serine/threonine protein kinase |
| 471 | GmaAffx.79121.1.S1_at | 0.373 | CA801737 | Transcription | Transcription | Zinc Finger | CCCH-type Zn-finger protein |
| | | | | | Factor | | |
| 472 | Gma.5578.3.S1_x_at | 0.372 | AW310572 | Metabolism | Cytochrome P450 | CYP98A | Cytochrome P450 CYP2 |
| | | | | | | | subfamily |
| 473 | GmaAffx.32011.1.S1_at | 0.372 | BI701198 | No Homology to | | | |
| | | | | Known Proteins | | | |
| 474 | Gma.8240.1.S1_at | 0.371 | BM092761 | Secondary Metabolism | | | Iron/ascorbate family |
| | | | | | | | oxidoreductases |
| 475 | GmaAffx.35729.1.S1_s_at | 0.37 | BE806047 | Unclassified - Hypothetical | | | Predicted E3 ubiquitin ligase |
| | | | | Protein Supported by | | | |
| | | | | cDNA | | | |
| 476 | GmaAffx.92462.1.S1_at | 0.37 | CF808187 | Transcription | Transcription | Zinc Finger | FOG: Predicted E3 |
| | | | | | Factor | | ubiquitin ligase |
| 477 | Gma.10615.1.A1_at | 0.37 | BU545626 | Transcription | Transcription | Basic Helix- | |
| | | | | | Factor | Loop-Helix | |
| | | | | | | (bHLH) | |
| 478 | GmaAffx.61272.1.S1_at | 0.369 | BI972394 | No Homology to | | | |
| | | | | Known Proteins | | | |
| 479 | GmaAffx.6008.1.S1_at | 0.369 | BU550119 | Transcription | Transcription | Basic Helix- | |
| | | | | | Factor | Loop-Helix | |
| | | | | | | (bHLH) | |
| 480 | GmaAffx.44716.1.A1_at | 0.368 | BI969342 | Disease & Defense | | | |
| 481 | GmaAffx.2745.1.S1_at | 0.368 | BF009213 | Unclassified - Hypothetical | | | Acetylglucosaminyltransferase |
| | | | | Protein Supported by | | | EXT1/exostosin 1 |
| | | | | cDNA | | | |
| 482 | Gma.5104.1.S1_at | 0.368 | BE611202 | Unclassified - Hypothetical | | | |
| | | | | Protein Supported by | | | |
| | | | | cDNA | | | |
| 483 | Gma.1332.1.S1_x_at | 0.368 | BI970666 | Secondary Metabolism | Ethylene | | 1-aminocyclopropane- |
| | | | | | Biosynthesis | | 1-carboxylate synthase, |
| | | | | | | | and related proteins |
| 484 | Gma.10331.1.A1_at | 0.368 | CD402553 | Unclassified - Protein with | | | Macrophage migration |
| | | | | Unknown Function | | | inhibitory factor |
| 485 | GmaAffx.91917.1.S1_s_at | 0.368 | CF807642 | Unclassified - Hypothetical | | | |
| | | | | Protein Supported by | | | |
| | | | | cDNA | | | |
| 486 | GmaAffx.4445.1.A1_at | 0.368 | BU547894 | Transcription | Transcription | AP2/EREBP | |
| | | | | | Factor | | |
| 487 | GmaAffx.92537.1.S1_at | 0.367 | CF808262 | Energy | | | Thioredoxin |
| 488 | GmaAffx.44471.1.A1_at | 0.367 | CA851833 | Unclassified - Hypothetical | | | |
| | | | | Protein NOT Supported by | | | |
| | | | | cDNA | | | |
| 489 | GmaAffx.86613.2.S1_at | 0.367 | BE024005 | Metabolism | | | Ghitaredoxin and related proteins |
| 490 | Gma.1263.1.S1_at | 0.366 | AY144180.1 | Metabolism | | | Ca2+/ |
| | | | | | | | calmodulin-dependent |
| | | | | | | | protein kinase, |
| | | | | | | | EF-Hand protein |
| | | | | | | | superfamily |
| 491 | GmaAffx.87493.1.S1_at | 0.366 | AW781752 | Unclassified - Hypothetical | | | |
| | | | | Protein NOT Supported by | | | |
| | | | | cDNA | | | |
| 492 | GmaAffx.7771.1.S1_at | 0.366 | BE022913 | Transporter | Inorganic Solute | Putative sulfate | Sulfate/bicarbonate/oxalate exchanger |
| | | | | | Cotransporters | transporter family | SAT-1 and related transporters |
| | | | | | | | (SLC26 family) |
| 493 | GmaAffx.93214.1.S1_at | 0.366 | CF808939 | No Homology to | | | |
| | | | | Known Proteins | | | |
| 494 | GmaAffx.36972.1.S1_at | 0.366 | BQ079450 | Unclassified - Hypothetical | | | |
| | | | | Protein NOT Supported by | | | |
| | | | | cDNA | | | |
| 495 | Gma.1979.1.S1_a_at | 0.366 | CD403280 | Unclassified - Protein with | | | Multitransmembrane protein |
| | | | | Unknown Function | | | |
| 496 | Gma.8160.1.S1_at | 0.366 | BI787214 | Unclassified - Hypothetical | | | |
| | | | | Protein NOT Supported by | | | |
| | | | | cDNA | | | |
| 497 | GmaAffx.4936.1.S1_at | 0.365 | AW666270 | Transporter | | | Multidrug/pheromone exporter, |
| | | | | | | | ABC superfamily |
| 498 | GmaAffx.23475.1.A1_at | 0.365 | BU548049 | Unclassified - Hypothetical | | | |
| | | | | Protein Supported by | | | |
| | | | | cDNA | | | |
| 499 | GmaAffx.68060.2.A1_at | 0.365 | AW396457 | No Homology to | | | |
| | | | | Known Proteins | | | |
| 500 | Gma.3511.4.S1_at | 0.364 | BMM526875 | Cell Structure | | | |
| 501 | Gma.13013.2.S1_a_at | 0.364 | AW457857 | Unclassified - Hypothetical | | | |
| | | | | Protein Supported by | | | |
| | | | | cDNA | | | |
| 502 | GmaAffx.81851.1.S1_at | 0.364 | AW706751 | Unclassified - Hypothetical | | | |
| | | | | Protein Supported by | | | |
| | | | | cDNA | | | |
| 503 | GmaAffx.81399.1.S1_at | 0.364 | AW568772 | Cell Growth & Division | | | |
| 504 | Gma.2342.1.S1_at | 0.363 | Z46953.1 | Transcription | Transcription | Heat Shock | Heat shock transcription factor |
| | | | | | Factor | | |
| 505 | GmaAffx.74390.1.A1_at | 0.363 | BU544169 | Protein Destination & Storage | | | Molecular chaperone |
| | | | | | | | (DnaJ superfamily) |
| 506 | Gma.6836.1.A1_at | 0.363 | AW349121 | Unclassified - Hypothetical | | | Predicted pyroglutamyl peptidase |
| | | | | Protein Supported by | | | |
| | | | | cDNA | | | |
| 507 | GmaAffx.37477.1.S1_s_at | 0.363 | BQ081355 | Metabolism | | | Branched chain aminotransferase |
| | | | | | | | BCAT1, pyridoxal phosphate enzymes |
| | | | | | | | type IV superfamily |
| 508 | GmaAffx.75740.1.S1_at | 0.363 | BG157373 | Unclassified - Hypothetical | | | |
| | | | | Protein NOT Supported by | | | |
| | | | | cDNA | | | |
| 509 | GmaAffx.48042.1.S1_at | 0.362 | AI960210 | Transporter | | | Aquaporin |
| | | | | | | | (major intrinsic protein family) |
| 510 | GmaAffx.23295.1.S1_at | 0.362 | BI968028 | Disease & Defense | | | |
| 511 | GmaAffx.17892.1.S1_at | 0.362 | AI442822 | Unclassified - Hypothetical | | | |
| | | | | Protein Supported by | | | |
| | | | | cDNA | | | |
| 512 | Gma.5309.1.S1_at | 0.362 | BM092263 | Unclassified - Hypothetical | | | |
| | | | | Protein NOT Supported by | | | |
| | | | | cDNA | | | |
| 513 | GmaAffx.25598.1.S1_at | 0.362 | AW311255 | No Homology to | | | |
| | | | | Known Proteins | | | |
| 514 | GmaAffx.84261.1.S1_at | 0.362 | BI701092 | Transcription | Transcription | AP2/EREBP | |
| | | | | | Factor | | |
| 515 | Gma.14075.1.S1_at | 0.362 | CD415333 | Protein Destination & Storage | Storage Protein | | Ca2+-independent phospholipase |
| | | | | | | | A2 |
| 516 | GmaAffx.82582.1.S1_at | 0.361 | BM139949 | No Homology to | | | |
| | | | | Known Proteins | | | |
| 517 | GmaAffx.93342.1.S1_s_at | 0.361 | CF809067 | Disease & Defense | | | Glutathione peroxidase |
| 518 | Gma.11269.1.S1_at | 0.36 | CD395046 | Transporter | | | |
| 519 | GmaAffx.83715.1.S1_at | 0.36 | BI785710 | Transporter | | | Glutaredoxin-related protein |
| 520 | Gma.1188.2.S1_at | 0.36 | BG406072 | Cell Growth & Division | | | |
| 521 | Gma.7227.2.S1_a_at | 0.36 | AW759183 | Protein Destination & Storage | | | Ubiquitin- |
| | | | | | | | protein ligase |
| 522 | GmaAffx.92127.1.S1_s_at | 0.359 | CF807852 | Transcription | Transcription | AP2/EREBP | |
| | | | | | Factor | | |
| 523 | GmaAffx.28397.1.S1_at | 0.359 | BM568229 | Metabolism | | | |
| 524 | Gma.364.1.A1_at | 0.359 | AW309875 | Unclassified - Hypothetical | | | |
| | | | | Protein Supported by | | | |
| | | | | cDNA | | | |
| 525 | GmaAffx.86675.1.S1_at | 0.359 | BI424294 | No Homology to | | | |
| | | | | Known Proteins | | | |
| 526 | Gma.5331.1.S1_a_at | 0.359 | BI892702 | Transcription | Transcription | NAC Domain | |
| | | | | | Factor | | |
| 527 | GmaAffx.5285.1.S1_at | 0.359 | BM523736 | Metabolism | | | |
| 528 | Gma.6213.1.S1_at | 0.358 | CD401652 | Protein Destination & Storage | | | Glutathione S-transferase |
| 529 | GmaAffx.84607.1.S1_at | 0.357 | BF596776 | Unclassified - Hypothetical | | | |
| | | | | Protein Supported by | | | |
| | | | | cDNA | | | |
| 530 | GmaAffx.11857.1.A1_at | 0.357 | AW351021 | Unclassified - Hypothetical | | | |
| | | | | Protein Supported by | | | |
| | | | | cDNA | | | |
| 531 | Gma.17007.1.S1_at | 0.357 | BE820533 | No Homology to | | | |
| | | | | Known Proteins | | | |
| 532 | GmaAffx.37615.1.S1_at | 0.357 | BQ454150 | No Homology to | | | |
| | | | | Known Proteins | | | |
| 533 | GmaAffx.15471.2.S1_at | 0.357 | BI784829 | Transcription | Transcription | MYB | Transcription factor, Myb |
| | | | | | Factor | | superfamily |
| 534 | Gma.5294.1.S1_at | 0.357 | BE658252 | Unclassified - Hypothetical | | | |
| | | | | Protein Supported by | | | |
| | | | | cDNA | | | |
| 535 | GmaAffx.43774.1.A1_at | 0.357 | AW349173 | Cell Structure | | | |
| 536 | GmaAffx.74933.1.S1_at | 0.355 | CA935084 | Unclassified - Protein with | | | Aspartyl protease |
| | | | | Unknown Function | | | |
| 537 | GmaAffx.37458.2.S1_at | 0.355 | BQ079820 | Unclassified - Hypothetical | | | |
| | | | | Protein NOT Supported by | | | |
| | | | | cDNA | | | |
| 538 | Gma.2602.1.S1_at | 0.355 | BU577743 | No Homology to | | | |
| | | | | Known Proteins | | | |
| 539 | Gma.4336.1.S1_at | 0.355 | BQ079907 | Unclassified - Hypothetical | | | |
| | | | | Protein NOT Supported by | | | |
| | | | | cDNA | | | |
| 540 | Gma.5478.1.S1_at | 0.353 | AW350606 | Metabolism | | | |
| 541 | Gma.10701.1.S1_at | 0.353 | BF596612 | Unclassified - Hypothetical | | | |
| | | | | Protein Supported by | | | |
| | | | | cDNA | | | |
| 542 | GmaAffx.37055.1.A1_at | 0.353 | BM142939 | Metabolism | | | UDP-glucuronosyl and UDP- |
| | | | | | | | glucosyl transferase |
| 543 | Gma.11119.2.S1_s_at | 0.353 | CF920743 | Disease & Defense | | | |
| 544 | GmaAffx.13016.1.S1_at | 0.352 | BF069324 | No Homology to | | | |
| | | | | Known Proteins | | | |
| 545 | Gma.8331.1.S1_at | 0.352 | BE659403 | Metabolism | | | Aldehyde dehydrogenase |
| 546 | Gma.11277.1.S1_at | 0.351 | CA802310 | Protein Destination & Storage | | | |
| 547 | Gma.10365.1.S1_at | 0.351 | BG725377 | Unclassified - Hypothetical | | | |
| | | | | Protein Supported by | | | |
| | | | | cDNA | | | |
| 548 | GmaAffx.70210.1.S1_at | 0.351 | BI699931 | Transcription | Transcription | Unclass ified | |
| | | | | | Factor | | |
| 549 | Gma.14567.1.S1_at | 0.35 | BE824123 | Disease & Defense | | | |
| 550 | Gma.14325.1.S1_at | 0.35 | BI700686 | Disease & Defense | | | Ca2+-independent phospholipase |
| | | | | | | | A2 |
| 551 | Gma.4372.1.S1_at | 0.35 | AI443505 | Secondary Metabolism | | | Reductases with broad range of |
| | | | | | | | substrate specificities |
| 552 | GmaAffx.66676.1.S1_at | 0.35 | BQ610288 | Unclassified - Protein with | | | |
| | | | | Unknown Function | | | |
| 553 | Gma.13418.1.S1_at | 0.35 | BM143894 | Transporter | | | |
| 554 | Gma.17688.1.S1_at | 0.349 | CA937360 | Transcription | Transcription | Basic Helix- | |
| | | | | | Factor | Loop-Helix | |
| | | | | | | (bHLH) | |
| 555 | GmaAffx.92166.1.S1_s_at | 0.349 | CF806313 | Signal Transduction | Unclassified | | Aspartyl protease |
| 556 | GmaAffx.37574.1.S1_s_at | 0.349 | BQ298504 | Secondary Metabolism | | | Hydroxyindole-O-methyltransferase |
| | | | | | | | and related SAM-dependent |
| | | | | | | | methyltransferases |
| 557 | Gma.382.1.A1_at | 0.349 | AW349068 | Metabolism | | | Arylacetamide deacetylase |
| 558 | Gma.752.I.A1_at | 0.349 | AW432463 | Transcription | Transcription | Basic Helix- | |
| | | | | | Factor | Loop-Helix | |
| | | | | | | (bHLH) | |
| 559 | GmaAffx.32765.1.S1_at | 0.348 | BE024072 | No Homology to | | | |
| | | | | Known Proteins | | | |
| 560 | Gma.968.1.S1_at | 0.348 | D28876.1 | Protein Destination & Storage | | | Asparaginyl peptidases |
| 561 | Gma.10756.1.S1_s_at | 0.348 | BE822092 | Unclassified - Hypothetical | | | |
| | | | | Protein Supported by | | | |
| | | | | cDNA | | | |
| 562 | GmaAffx.23276.1.S1_at | 0.347 | AI856198 | Transporter | | | Amino acid transporters |
| 563 | GmaAffx.50673.2.S1_s_at | 0.347 | BI787147 | Transcription | Transcription | NAC Domain | |
| | | | | | Factor | | |
| 564 | GmaAffx.24359.1.A1_at | 0.346 | BU544840 | Metabolism | | | |
| 565 | GmaAffx.57469.1.S1_at | 0.346 | AW351054 | Unclassified - Hypothetical | | | |
| | | | | Protein Supported by | | | |
| | | | | cDNA | | | |
| 566 | GmaAffx.91561.1.S1_s_at | 0.346 | CF807286 | Transporter | | | Apolipoprotein D/Lipocalin |
| 567 | Gma.5084.1.S1_at | 0.346 | AW309848 | Protein Destination & Storage | Storage Protein | | |
| 568 | Gma.3888.2.S1_at | 0.345 | BM139532 | Unclassified - Hypothetical | | | Aldehyde reductase |
| | | | | Protein NOT Supported by | | | |
| | | | | cDNA | | | |
| 569 | Gma.16359.1.A1_at | 0.344 | BI970359 | Metabolism | | | GPI-alpha-mannosyl-transferase III |
| | | | | | | | (GPI10/PIG-B) involved in |
| | | | | | | | glycosylphosphatidylinositol |
| | | | | | | | anchor biosynthesis |
| 570 | Gma.8530.1.S1_at | 0.344 | AF109720.1 | Protein Destination & Storage | Storage Protein | | |
| 571 | Gma.9056.1.S1_at | 0.343 | CA819787 | Metabolism | | | Acyl-CoA-binding protein |
| 572 | GmaAffx.70056.1.S1_at | 0.343 | BU765705 | No Homology to | | | |
| | | | | Known Proteins | | | |
| 573 | Gma.10612.1.S1_s_at | 0.343 | BG650314 | No Homology to | | | |
| | | | | Known Proteins | | | |
| 574 | GmaAffx.1301.52.S1_at | 0.343 | BG510366 | Unclassified - Hypothetical | | | |
| | | | | Protein Supported by | | | |
| | | | | cDNA | | | |
| 575 | GmaAffx.65217.1.A1_at | 0.343 | BU543988 | Metabolism | | | Acyl-CoA-binding protein |
| 576 | Gma.17729.1.S1_s_at | 0.343 | CD393936 | No Homology to | | | |
| | | | | Known Proteins | | | |
| 577 | GmaAffx.15983.2.S1_s_at | 0.343 | AW279540 | Signal Transduction | Protein Phosphatase | | Serine/threonine protein phosphatase |
| 578 | GmaAffx.83964.1.S1_at | 0.342 | BU082747 | Unclassified - Protein with | | | Protein involved in |
| | | | | Unknown Function | | | mRNA turnover and stability |
| 579 | GmaAffx.33408.1.S1_at | 0.342 | BU547044 | No Homology to | | | |
| | | | | Known Proteins | | | |
| 580 | Gma.1137.1.S1_x_at | 0.342 | BF067710 | Metabolism | | | |
| 581 | GmaAffx.46681.1.S1_at | 0.342 | BQ080794 | Unclassified - Hypothetical | | | |
| | | | | Protein Supported by | | | |
| | | | | cDNA | | | |
| 582 | Gma.5256.1.A1_at | 0.341 | CD408042 | No Homology to | | | |
| | | | | Known Proteins | | | |
| 583 | Gma.11277.1.S1_x_at | 0.341 | CA802310 | Protein Destination & Storage | | | |
| 584 | Gma.2952.1.S1_at | 0.341 | BM270250 | No Homology to | | | |
| | | | | Known Proteins | | | |
| 585 | Gma.13035.1.S1_at | 0.341 | CD396536 | No Homology to | | | |
| | | | | Known Proteins | | | |
| 586 | Gma.15863.1.S1_s_at | 0.34 | CD413163 | Metabolism | | | |
| 587 | Gma.5255.1.S1_at | 0.34 | AI736533 | Unclassified - Hypothetical | | | |
| | | | | Protein Supported by | | | |
| | | | | cDNA | | | |
| 588 | Gma.12902.1.A1_at | 0.34 | CD395017 | Metabolism | | | Hydroxyindole-O-methyltransferase |
| | | | | | | | and related SAM-dependent |
| | | | | | | | methyltransferases |
| 589 | Gma.3917.1.S1_at | 0.34 | BE658782 | Cell Structure | | | Pectin |
| | | | | | | | acetylesterase and |
| | | | | | | | similar proteins |
| 590 | Gma.9907.1.S1_at | 0.339 | AW309518 | Transcription | Transcription | Basic Helix- | |
| | | | | | Factor | Loop-Helix | |
| | | | | | | (bHLH) | |
| 591 | GmaAffx.29254.1.S1_at | 0.338 | AW760361 | Transcription | Transcription | Basic Helix- | |
| | | | | | Factor | Loop-Helix | |
| | | | | | | (bHLH) | |
| 592 | GmaAffx.31862.1.S1_at | 0.338 | BE822501 | Unclassified - Hypothetical | | | |
| | | | | Protein Supported by | | | |
| | | | | cDNA | | | |
| 593 | GmaAffx.37930.1.A1_at | 0.338 | BU547709 | Cell Structure | | | |
| 594 | Gma.5578.4.S1_a_at | 0.338 | BE023932 | Metabolism | Cytochrome P450 | CYP98A | Cytochrome P450 CYP2 |
| | | | | | | | subfamily |
| 595 | GmaAffx.37458.1.S1_at | 0.338 | BU549419 | Unclassified - Protein with | | | |
| | | | | Unknown Function | | | |
| 596 | Gma.10824.1.S1_at | 0.338 | BE824257 | Unclassified - Hypothetical | | | Magnesium transporters: CorA family |
| | | | | Protein Supported by | | | |
| | | | | cDNA | | | |
| 597 | GmaAffx.93189.1.S1_at | 0.338 | CF808914 | Metabolism | | | Carbon-nitrogen hydrolase |
| 598 | Gma.4222.1.S1_at | 0.337 | BU083971 | Unclassified - Hypothetical | | | Arylacetamide deacetylase |
| | | | | Protein NOT Supported by | | | |
| | | | | cDNA | | | |
| 599 | GmaAffx.64075.1.S1_at | 0.337 | BI700868 | Unclassified - Hypothetical | | | |
| | | | | Protein Supported by | | | |
| | | | | cDNA | | | |
| 600 | GmaAffx.88945.1.S1_at | 0.337 | CK605738 | Unclassified - Protein with | | | |
| | | | | Unknown Function | | | |
| 601 | GmaAffx.91328.1.S1_at | 0.336 | CF807053 | Unclassified - Hypothetical | | | Leucine rich repeat proteins, |
| | | | | Protein Supported by | | | some proteins contain F-box |
| | | | | cDNA | | | |
| 602 | Gma.10827.2.S1_s_at | 0.335 | BQ612452 | Protein Destination & Storage | Storage Protein | | |
| 603 | GmaAffx.92070.1.S1_at | 0.335 | CF807049 | Cell Structure | | | O-methyltransferase |
| 604 | GmaAffx.10908.1.S1_at | 0.335 | BE822295 | Transcription | Transcription | Homeodomain | |
| | | | | | Factor | | |
| 605 | Gma.17610.1.S1_x_at | 0.334 | U50075.1 | Metabolism | | | |
| 606 | Gma.14361.1.A1_at | 0.333 | CD409843 | No Homology to | | | |
| | | | | Known Proteins | | | |
| 607 | Gma.12731.2.A1_s_at | 0.333 | CD395848 | Unclassified - Hypothetical | | | Aquaporin |
| | | | | Protein NOT Supported by | | | (major intrinsic protein family) |
| | | | | cDNA | | | |
| 608 | GmaAffx.93183.1.S1_s_at | 0.333 | CF808908 | Secondary Metabolism | | | S-adenosylmethionine decarboxylase |
| 609 | GmaAffx.46103.1.S1_at | 0.333 | BM091933 | Metabolism | | | Hydroxymethylglutaryl-CoA |
| | | | | | | | lyase |
| 610 | Gma.336.1.A1_at | 0.333 | AW310815 | Metabolism | Sulfur Metabolism | | |
| 611 | GmaAffx.41990.1.S1_at | 0.332 | CA936708 | No Homology to | | | |
| | | | | Known Proteins | | | |
| 612 | Gma.6782.2.S1_a_at | 0.332 | AW156813 | Unclassified - Hypothetical | | | |
| | | | | Protein Supported by | | | |
| | | | | cDNA | | | |
| 613 | GmaAffx.93182.1.S1_s_at | 0.332 | CF808907 | Secondary Metabolism | Ethylene | | Iron/ascorbate family |
| | | | | | Biosynthesis | | oxidoreductases |
| 614 | GmaAffx.62364.1.S1_at | 0.331 | AW733764 | Protein Destination & Storage | | | Dipeptidyl aminopeptidase |
| 615 | Gma.8312.1.A1_at | 0.331 | AW310254 | Unclassified - Protein with | | | |
| | | | | Unknown Function | | | |
| 616 | GmaAffx.86844.1.S1_at | 0.331 | BE822146 | Unclassified - Protein with | | | |
| | | | | Unknown Function | | | |
| 617 | Gma.4093.1.S1_at | 0.33 | BI700897 | Transporter | | | |
| 618 | GmaAffx.17098.1.S1_at | 0.33 | BM084976 | Disease & Defense | | | |
| 619 | GmaAffx.33778.I.S1_at | 0.329 | CA819035 | Protein Destination & Storage | Storage Protein | | |
| 620 | Gma.6550.3.S1_s_at | 0.329 | AW234450 | Metabolism | | | Hydroxyindole-O-methyltransferase |
| | | | | | | | and related SAM-dependent |
| | | | | | | | methyltransferases |
| 621 | Gma.1622.1.A1_s_at | 0.329 | BU090523 | Signal Transduction | Protein Kinase | | FOG: Transposon-encoded proteins with |
| | | | | | | | TYA, reverse transcriptase, integrase |
| | | | | | | | domains in various combinations |
| 622 | Gma.7107.1.S1_at | 0.329 | AI855972 | Unclassified - Protein with | | | |
| | | | | Unknown Function | | | |
| 623 | Gma.4340.2.S1_a_at | 0.328 | BE611760 | Secondary Metabolism | | | Hydroxyindole-O-methyltransferase |
| | | | | | | | and related |
| | | | | | | | SAM-dependent |
| | | | | | | | methyltransferases |
| 624 | Gma.4873.1.S1_at | 0.328 | BI969532 | Signal Transduction | Protein Kinase | | Serine/threonine protein kinase |
| 625 | Gma.13335.1.S1_at | 0.327 | CD417489 | Unclassified - Hypothetical | | | |
| | | | | Protein Supported by | | | |
| | | | | cDNA | | | |
| 626 | GmaAffx.2716.1.A1_at | 0.327 | BU547858 | Transcription | Transcription | Zinc Finger | Protein involved in |
| | | | | | Factor | | mRNA turnover and stability |
| 627 | Gma.6033.1.S1_at | 0.327 | BI967767 | Unclassified - Hypothetical | | | |
| | | | | Protein Supported by | | | |
| | | | | cDNA | | | |
| 628 | Gma.13418.3.S1_at | 0.327 | CA938391 | Transporter | | | |
| 629 | Gma.4897.1.S1_at | 0.326 | BI969641 | Unclassified - Protein with | | | Thioredoxin, nucleoredoxin and |
| | | | | Unknown Function | | | related proteins |
| 630 | Gma.2540.2.S1_s_at | 0.325 | BI787366 | No Homology to | | | |
| | | | | Known Proteins | | | |
| 631 | GmaAffx.93553.1.S1_s_at | 0.325 | CF809278 | Metabolism | | | |
| 632 | Gma.13296.3.S1_at | 0.324 | CD418313 | Transporter | | | |
| 633 | GmaAffx.91254.1.S1_x_at | 0.324 | CF806979 | Unclassified - Hypothetical | | | |
| | | | | Protein Supported by | | | |
| | | | | cDNA | | | |
| 634 | GmaAffx.33677.1.S1_at | 0.324 | CA782582 | No Homology to | | | |
| | | | | Known Proteins | | | |
| 635 | GmaAffx.84144.1.A1_s_at | 0.324 | CD414061 | No Homology to | | | |
| | | | | Known Proteins | | | |
| 636 | GmaAffx.63553.1.S1_at | 0.324 | BG352380 | Energy | | | Cytochrome b |
| 637 | GmaAffx.93189.1.S1_s_at | 0.324 | CF808914 | Metabolism | | | Carbon-nitrogen hydrolase |
| 638 | GmaAffx.47819.1.A1_s_at | 0.324 | CD416142 | Unclassified - Hypothetical | | | Predicted mitochondrial |
| | | | | Protein Supported by | | | ribosomal protein L23 |
| | | | | cDNA | | | |
| 639 | Gma.5097.1.A1_at | 0.323 | BI969945 | Unclassified - Hypothetical | | | |
| | | | | Protein Supported by | | | |
| | | | | cDNA | | | |
| 640 | Gma.1895.1.A1_at | 0.323 | AW309584 | Protein Destination & Storage | | | ER lumen protein retaining |
| | | | | | | | receptor |
| 641 | Gma.12393.3.S1_at | 0.323 | BM085488 | Secondary Metabolism | | | Beta, beta-carotene 15,15′-dioxygenase |
| | | | | | | | and related enzymes |
| 642 | Gma.15359.2.S1_a_at | 0.322 | AW781747 | Intracellular Traffic | | | Predicted Ca2+-dependent |
| | | | | | | | phospholipid-binding protein |
| 643 | GmaAffx.84566.1.S1_x_at | 0.322 | BI786849 | Transcription | Transcription | MYB | Transcription factor, Myb |
| | | | | | Factor | | superfamily |
| 644 | Gma,12211.2.S1_at | 0.322 | BQ299492 | Disease & Defense | | | |
| 645 | Gma.17805.1.A1_s_at | 0.321 | BI968729 | Disease & Defense | | | |
| 646 | Gma.5950.1.S1_s_at | 0.321 | BQ610563 | Disease & Defense | | | |
| 647 | GmaAffx.70959.1.S1_at | 0.32 | BE611219 | Unclassified - Hypothetical | | | Glucose-6-phosphate/phosphate and |
| | | | | Protein NOT Supported by | | | phosphoenolpyruvate/phosphate antiporter |
| | | | | cDNA | | | |
| 648 | Gma.8484.1.S1_at | 0.32 | AF197946.1 | Signal Transduction | Protein Kinase | | |
| 649 | Gma.6213.1.S1_s_at | 0.32 | CD401652 | Protein Destination & Storage | | | Glutathione S-transferase |
| 650 | Gma.1394.2.S1_a_at | 0.32 | BI470370 | Metabolism | | | |
| 651 | GmaAffx.24350.1.A1_at | 0.32 | BU544060 | No Homology to | | | |
| | | | | Known Proteins | | | |
| 652 | GmaAffx.81287.1.S1_at | 0.319 | BG789983 | Secondary Metabolism | | | Hydroxyindole-O-methyltransferase |
| | | | | | | | and related SAM-dependent |
| | | | | | | | methyltransferases |
| 653 | Gma.13361.1.S1_at | 0.318 | AW309932 | Unclassified - Protein with | | | |
| | | | | Unknown Function | | | |
| 654 | Gma.2712.1.S1_at | 0.318 | AJ003246.1 | Secondary Metabolism | | | Flavonol reductase/cinnamoyl-CoA |
| | | | | | | | reductase |
| 655 | GmaAffx.89867.1.A1_s_at | 0.317 | CK606556 | Metabolism | | | Predicted PhzC/PhzF-type epimerase |
| 656 | Gma.2302.1.S1_x_at | 0.317 | AW307490 | Disease & Defense | | | Cysteine proteinase Cathepsin L |
| 657 | Gma.1573.2.S1_a_at | 0.316 | BI974098 | Metabolism | | | |
| 658 | GmaAffx.66301.1.S1_at | 0.316 | BG510223 | Secondary Metabolism | Ethylene | | Iron/ascorbate family |
| | | | | | Biosynthesis | | oxidoreductases |
| 659 | GmaAffx.72986.1.S1_at | 0.315 | BI317536 | Metabolism | | | Inositol polyphosphate 5- |
| | | | | | | | phosphatase and related proteins |
| 660 | GmaAffx.1620.3.S1_s_at | 0.315 | AW132226 | No Homology to | | | |
| | | | | Known Proteins | | | |
| 661 | GmaAffx.80098.1.S1_at | 0.315 | BG042861 | Metabolism | | | Arylacetamide deacetylase |
| 662 | Gma.9013.1.S1_at | 0.315 | AF243368.1 | Disease & Defense | | | Glutathione S-transferase |
| 663 | Gma.821.1.S1_at | 0.315 | AF243371.1 | Metabolism | | | Glutathione S-transferase |
| 664 | Gma.16940.1.S1_at | 0.315 | AW310039 | Transcription | Transcription | Homeodomain | Transcription factor MEIS1 and |
| | | | | | Factor | | related HOX domain proteins |
| 665 | Gma.10950.1.S1_at | 0.315 | BI699798 | Unclassified - Hypothetical | | | |
| | | | | Protein Supported by | | | |
| | | | | cDNA | | | |
| 666 | Gma.157.1.S1_at | 0.314 | D86351.1 | Metabolism | Cytochrome P450 | CYP93D | Cytochrome P450 CYP2 |
| | | | | | | | subfamily |
| 667 | GmaAffx.26967.1.S1_at | 0.314 | BE022958 | Unclassified - Hypothetical | | | |
| | | | | Protein Supported by | | | |
| | | | | cDNA | | | |
| 668 | GmaAffx.34798.1.S1_at | 0.314 | BM143105 | Unclassified - Hypothetical | | | |
| | | | | Protein Supported by | | | |
| | | | | cDNA | | | |
| 669 | GmaAffx.53845.1.S1_at | 0.313 | BG406552 | Transporter | | | Sulfate/bicarbonate/oxalate exchanger SAT-1 |
| | | | | | | | and related transporters (SLC26 family) |
| 670 | GmaAffx.28539.1.S1_at | 0.313 | BQ080184 | Disease & Defense | | | Ca2+-independent phospholipase A2 |
| 671 | Gma.11205.1.S1_a_at | 0.313 | BF066553 | Metabolism | | | Transketolase |
| 672 | GmaAffx.9127.1.S1_s_at | 0.313 | BI317582 | Transcription | Transcription | Zinc Finger | FOG: |
| | | | | | Factor | | Predicted E3 ubiquitin ligase |
| 673 | GmaAffx.3706.1.S1_at | 0.313 | BE821261 | Unclassified - Hypothetical | | | HGG motif-containing |
| | | | | Protein NOT Supported by | | | thioesterase |
| | | | | cDNA | | | |
| 674 | Gma.4526.1.S1_at | 0.312 | BI967711 | Transcription | Transcription | Zinc Finger | FOG: Zn-finger |
| | | | | | Factor | | |
| 675 | Gma.1704.1.S1_at | 0.312 | AW349971 | Metabolism | | | Glutaredoxin and related proteins |
| 676 | Gma.398.2.S1_at | 0.312 | BU925900 | Metabolism | | | Homocysteine S-methyltransferase |
| 677 | GmaAffx.82564.1.S1_s_at | 0.312 | BG047418 | No Homology to | | | |
| | | | | Known Proteins | | | |
| 678 | Gma.9622.1.S1_x_at | 0.311 | BQ610840 | Metabolism | | | Hydroxyindole-O-methyltransferase |
| | | | | | | | and related SAM-dependent |
| | | | | | | | methyltransferases |
| 679 | Gma.9205.1.S1_at | 0.31 | BM528605 | Unclassified - Hypothetical | | | |
| | | | | Protein Supported by | | | |
| | | | | cDNA | | | |
| 680 | Gma.2776.1.S1_at | 0.31 | BI968513 | Secondary Metabolism | Ethylene | | Iron/ascorbate family |
| | | | | | Biosynthesis | | oxidoreductases |
| 681 | Gma.6550.2.S1_x_at | 0.309 | BM086044 | Metabolism | | | Hydroxyindole-O- |
| | | | | | | | methyltransferase |
| | | | | | | | and related |
| | | | | | | | SAM-dependent |
| | | | | | | | methyltransferases |
| 682 | Gma.3581.2.S1_x_at | 0.309 | CD398783 | Unclassified - Hypothetical | | | Ca2+-dependent lipid-binding |
| | | | | Protein NOT Supported by | | | protein CLB1/vesicle |
| | | | | cDNA | | | protein vp115/Granuphilin A, |
| | | | | | | | contains C2 domain |
| 683 | GmaAffx.75594.1.S1_at | 0.309 | AW307139 | Secondary Metabolism | | | Multicopper oxidases |
| 684 | GmaAffx.14030.1.S1_at | 0.308 | BE021503 | No Homology to | | | |
| | | | | Known Proteins | | | |
| 685 | GmaAffx.87101.1.S1_at | 0.308 | BM309451 | No Homology to | | | |
| | | | | Known Proteins | | | |
| 686 | GmaAffx.55362.1.S1_at | 0.308 | BE475283 | Unclassified - Hypothetical | | | Reticulon |
| | | | | Protein Supported by | | | |
| | | | | cDNA | | | |
| 687 | Gma.79.4.S1_s_at | 0.308 | AI443819 | Unclassified - Hypothetical | | | |
| | | | | Protein Supported by | | | |
| | | | | cDNA | | | |
| 688 | Gma.8556.1.S1_at | 0.308 | BU764573 | Transporter | | | Amino acid transporters |
| 689 | GmaAffx.82195.1.S1_at | 0.308 | BM528489 | Unclassified - Hypothetical | | | |
| | | | | Protein NOT Supported by | | | |
| | | | | cDNA | | | |
| 690 | Gma.3450.1.S1_at | 0.307 | BE657895 | Unclassified - Hypothetical | | | |
| | | | | Protein Supported by | | | |
| | | | | cDNA | | | |
| 691 | GmaAffx.64261.1.A1_at | 0.307 | BI969080 | Unclassified - Protein with | | | 1,4-benzoquinone reductase-like; Trp |
| | | | | Unknown Function | | | repressor binding protein-like/ |
| | | | | | | | protoplast-secreted protein |
| 692 | GmaAffx.90515.1.S1_s_at | 0.306 | CF806240 | Unclassified - Hypothetical | | | Predicted pyroglutamyl peptidase |
| | | | | Protein Supported by | | | |
| | | | | cDNA | | | |
| 693 | Gma.4578.1.S1_s_at | 0.306 | BE802809 | Transporter | | | Plasma membrane H+-transporting |
| | | | | | | | ATPase |
| 694 | GmaAffx.47739.1.S1_at | 0.305 | BM522736 | Unclassified - Protein with | | | |
| | | | | Unknown Function | | | |
| 695 | Gma.3161.2.S1_at | 0.305 | CD400025 | Secondary Metabolism | | | Iron/ascorbate family |
| | | | | | | | oxidoreductases |
| 696 | GmaAffx.93116.1.S1_at | 0.305 | CF806290 | Disease & Defense | | | |
| 697 | GmaAffx.5255.1.S1_at | 0.305 | BG362929 | Transcription | Transcription | AP2/EREBP | |
| | | | | | Factor | | |
| 698 | Gma.3976.1.S1_at | 0.304 | BI972268 | No Homology to | | | |
| | | | | Known Proteins | | | |
| 699 | Gma.12641.1.A1_at | 0.304 | CD393495 | No Homology to | | | |
| | | | | Known Proteins | | | |
| 700 | GmaAffx.72479.1.S1_at | 0.303 | BG043825 | Transcription | Transcription | AP2/EREBP | Uncharacterized conserved protein |
| | | | | | Factor | | |
| 701 | GmaAffx.48438.1.S1_at | 0.302 | AW350858 | Protein Destination & Storage | | | Molecular chaperone |
| | | | | | | | (DnaJ superfamily) |
| 702 | Gma.6550.2.S1_s_at | 0.302 | BM086044 | Metabolism | | | Hydroxyindole-O-methyltransferase |
| | | | | | | | and related SAM-dependent |
| | | | | | | | methyltransferases |
| 703 | GmaAffx.57748.1.S1_at | 0.302 | AW306274 | No Homology to | | | |
| | | | | Known Proteins | | | |
| 704 | Gma.3751.1.S1_at | 0.301 | AW350303 | Unclassified - Hypothetical | | | |
| | | | | Protein NOT Supported by | | | |
| | | | | cDNA | | | |
| 705 | GmaAffx.16207.1.S1_at | 0.301 | BE821188 | Cell Structure | | | Beta-glucosidase, lactase |
| | | | | | | | phlorizinhydrolase, and related |
| | | | | | | | proteins |
| 706 | Gma.15359.1.S1_x_at | 0.3 | BM271008 | Unclassified - Protein with | | | Predicted Ca2+-dependent |
| | | | | Unknown Function | | | phospholipid-binding protein |
| 707 | GmaAffx.60672.1.S1_at | 0.299 | BU080558 | No Homology to | | | |
| | | | | Known Proteins | | | |
| 708 | Gma.6462.2.S1_a_at | 0.299 | BE555263 | Unclassified - Hypothetical | | | |
| | | | | Protein NOT Supported by | | | |
| | | | | cDNA | | | |
| 709 | GmaAffx.9926.1.A1_at | 0.299 | CD411291 | No Homology to | | | |
| | | | | Known Proteins | | | |
| 710 | GmaAffx.78297.1.S1_at | 0.299 | BM731185 | Unclassified - Hypothetical | | | FOG: |
| | | | | Protein Supported by | | | Predicted E3 ubiquitin ligase |
| | | | | cDNA | | | |
| 711 | Gma.2302.1.S1_at | 0.299 | AW307490 | Disease & Defense | | | Cysteine proteinase Cathepsin L |
| 712 | Gma.73.1.S1_at | 0.298 | AF098662.1 | Protein Destination & Storage | | | |
| 713 | Gma.6151.1.S1_at | 0.298 | BQ611806 | Metabolism | | | Flavin-containing monooxygenase |
| 714 | Gma.17610.1.S1_at | 0.298 | U50075.1 | Metabolism | | | |
| 715 | Gma.13187.1.S1_at | 0.298 | BG839239 | Unclassified - Hypothetical | | | Uncharacterized conserved protein |
| | | | | Protein Supported by | | | |
| | | | | cDNA | | | |
| 716 | GmaAffx.76696.1.A1_at | 0.297 | BE824386 | No Homology to | | | |
| | | | | Known Proteins | | | |
| 717 | GmaAffx.89060.1.S1_at | 0.296 | CK605664 | Metabolism | Cytochrome P450 | CYP93D | Cytochrome P450 CYP2 |
| | | | | | | | subfamily |
| 718 | GmaAffx.89772.10.A1_s_at | 0.296 | CK606097 | Disease & Defense | | | |
| 719 | GmaAffx.92112.1.S1_s_at | 0.295 | CF807837 | No Homology to | | | |
| | | | | Known Proteins | | | |
| 720 | Gma.15093.1.S1_at | 0.295 | CD400374 | Protein Destination & Storage | | | Glutathione S-transferase |
| 721 | GmaAffx.93157.1.S1_s_at | 0.295 | CF808882 | Metabolism | | | Aldo/keto reductase family |
| | | | | | | | proteins |
| 722 | GmaAffx.89075.1.S1_s_at | 0.295 | CK606454 | No Homology to | | | |
| | | | | Known Proteins | | | |
| 723 | Gma.6623.1.S1_at | 0.294 | BI702268 | Unclassified - Protein with | | | 1,4-benzoquinone reductase-like; Trp |
| | | | | Unknown Function | | | repressor binding |
| | | | | | | | protein-like/protoplast-secreted |
| | | | | | | | protein |
| 724 | Gma.4305.1.S1_at | 0.294 | AW350687 | Disease & Defense | | | |
| 725 | GmaAffx.38555.2.S1_at | 0.294 | BI425873 | No Homology to | | | |
| | | | | Known Proteins | | | |
| 726 | GmaAffx.91037.1.S1_s_at | 0.294 | CF806762 | Secondary Metabolism | | | Methyltransferase |
| 727 | GmaAffx.74300.1.S1_at | 0.294 | BU080963 | Disease & Defense | | | |
| 728 | GmaAffx.18113.2.S1_s_at | 0.294 | AW706795 | Unclassified - Protein with | Hormone Induced | | |
| | | | | Unknown Function | | | |
| 729 | GmaAffx.12014.1.A1_at | 0.294 | CA802431 | Unclassified - Hypothetical | | | NADH pyrophosphatase I of the |
| | | | | Protein Supported by | | | Nudix family of hydrolases |
| | | | | cDNA | | | |
| 730 | GmaAffx.89772.8.S1_at | 0.294 | CK605778 | Metabolism | | | |
| 731 | GmaAffx.61311.1.S1_at | 0.294 | CA936951 | Transporter | | | Amino acid transporters |
| 732 | GmaAffx.42732.1.S1_at | 0.294 | BU765857 | Protein Destination & Storage | Ubiquitin | | FOG: Predicted E3 |
| | | | | | Degradation | | ubiquitin ligase |
| 733 | GmaAffx.50677.1.S1_s_at | 0.293 | BI971744 | No Homology to | | | |
| | | | | Known Proteins | | | |
| 734 | Gma.3963.2.S1_at | 0.293 | BE608192 | Unclassified - Hypothetical | | | Zn-finger protein |
| | | | | Protein NOT Supported by | | | |
| | | | | cDNA | | | |
| 735 | Gma.2718.1.S1_at | 0.293 | CD406302 | Transporter | | | Multidrug resistance-associated protein/ |
| | | | | | | | mitoxantrone resistance protein, ABC |
| | | | | | | | superfamily |
| 736 | Gma.6640.1.S1_at | 0.292 | BQ628278 | Disease & Defense | | | |
| 737 | Gma.7454.1.S1_a_at | 0.292 | CD405901 | Transcription | Transcription | AUX/IAA | |
| | | | | | Factor | | |
| 738 | Gma.9236.1.S1_at | 0.291 | BG405642 | Metabolism | Amino Acid | | Spermidine synthase |
| | | | | | Biosynthesis/ | | |
| | | | | | Metabolism | | |
| 739 | Gma.6895.1.S1_a_at | 0.291 | BU545788 | Metabolism | | | Alanine-glyoxylate |
| | | | | | | | aminotransferase AGT2 |
| 740 | Gma.5733.1.S1_at | 0.291 | CD402671 | Signal Transduction | Calcium Binding | | Ca2+-binding protein (centrin/ |
| | | | | | | | caltractin), EF-Hand superfamily |
| | | | | | | | protein |
| 741 | GmaAffx.18953.1.S1_at | 0.291 | BM178798 | No Homology to | | | |
| | | | | Known Proteins | | | |
| 742 | GmaAffx.90703.1.A1_at | 0.29 | CF809087 | Disease & Defense | | | |
| 743 | GmaAffx.79390.2.S1_at | 0.29 | AW597383 | Transcription | Transcription | Basic Leucine | FOG: Armadillo/beta- |
| | | | | | Factor | Zipper (bZIP) | catenin-like repeats |
| 744 | Gma.6310.1.S1_at | 0.29 | BQ612831 | Signal Transduction | MAPKKK | MEKK | MEKK and related serine/threonine |
| | | | | | | | protein kinases |
| 745 | GmaAffx.31838.1.S1_at | 0.289 | BU547835 | Secondary Metabolism | Flavonoid | | Flavonol reductase/cinnamoyl-CoA |
| | | | | | Biosynthesis | | reductase |
| 746 | Gma.15605.1.S1_at | 0.288 | BM143798 | Transcription | Transcription | AP2/EREBP | |
| | | | | | Factor | | |
| 747 | Gma.17835.1.S1_at | 0.288 | BE822840 | Metabolism | | | |
| 748 | Gma.8394.1.S1_at | 0.288 | CD392332 | Disease & Defense | | | Prohibitins and stomatins of the |
| | | | | | | | PID superfamily |
| 749 | Gma.6264.1.S1_at | 0.288 | BQ612558 | Unclassified - Hypothetical | | | |
| | | | | Protein Supported by | | | |
| | | | | cDNA | | | |
| 750 | Gma.6630.1.S1_s_at | 0.288 | BG652851 | Unclassified - Hypothetical | | | |
| | | | | Protein Supported by | | | |
| | | | | cDNA | | | |
| 751 | Gma.2388.1.S1_at | 0.288 | CD416539 | No Homology to | | | |
| | | | | Known Proteins | | | |
| 752 | GmaAffx.727.1.S1_at | 0.287 | BE804565 | Unclassified - Protein with | | | Ubiquinonebiosynthesis |
| | | | | Unknown Function | | | methyltransferase COQ5 |
| 753 | Gma.13000.1.S1_at | 0.287 | CD415111 | Unclassified - Hypothetical | | | |
| | | | | Protein Supported by | | | |
| | | | | cDNA | | | |
| 754 | GmaAffx.11049.1.S1_at | 0.287 | BU545140 | Unclassified - Protein with | | | Phosphatidylethanolamine |
| | | | | Unknown Function | | | binding protein |
| 755 | Gma.11731.1.A1_at | 0.287 | CD392320 | Unclassified - Protein with | | | |
| | | | | Unknown Function | | | |
| 756 | Gma.5578.1.S1_x_at | 0.287 | BE659192 | Metabolism | Cytochrome P450 | CYP98A | Cytochrome P450 CYP2 |
| | | | | | | | subfamily |
| 757 | GmaAffx.93607.1.S1_s_at | 0.286 | CF809332 | Unclassified - Protein with | | | |
| | | | | Unknown Function | | | |
| 758 | GmaAffx.91185.1.S1_at | 0.286 | CF806910 | Cell Structure | | | Pectin acetylesterase |
| | | | | | | | and similar proteins |
| 759 | Gma.11277.2.S1_a_at | 0.286 | BI701238 | Unclassified - Hypothetical | | | |
| | | | | Protein NOT Supported by | | | |
| | | | | cDNA | | | |
| 760 | Gma.6033.2.S1_at | 0.286 | AI416728 | Unclassified - Hypothetical | | | |
| | | | | Protein | | | |
| | | | | Supported by | | | |
| | | | | cDNA | | | |
| 761 | GmaAffx.478.2.S1_at | 0.285 | BU763963 | Transcription | Transcription | Homeodomain | Transcription factor MEIS1 and |
| | | | | | Factor | | related HOX domain proteins |
| 762 | GmaAffx.71711.1.S1_at | 0.285 | AI988272 | No Homology to | | | |
| | | | | Known Proteins | | | |
| 763 | Gma.3345.1.S1_at | 0.285 | AW394524 | Metabolism | | | Alcohol dehydrogenase, class III |
| 764 | GmaAffx.84338.1.S1_at | 0.285 | AI856789 | Unclassified - Hypothetical | | | Predicted E3 ubiquitin ligase |
| | | | | Protein Supported by | | | |
| | | | | cDNA | | | |
| 765 | GmaAffx.90069.1.S1_at | 0.284 | CF805794 | Unclassified - Hypothetical | | | Leucine rich repeat proteins, |
| | | | | Protein Supported by | | | some proteins contain F-box |
| | | | | cDNA | | | |
| 766 | GmaAffx.75697.1.S1_s_at | 0.284 | BU090518 | Unclassified - Hypothetical | | | |
| | | | | Protein Supported by | | | |
| | | | | cDNA | | | |
| 767 | Gma.6187.1.S1_at | 0.283 | BF324343 | No Homology to | | | |
| | | | | Known Proteins | | | |
| 768 | Gma.12211.1.S1_at | 0.283 | AW394548 | Disease & Defense | | | |
| 769 | Gma.3880.1.S1_at | 0.283 | BE659305 | Unclassified - Hypothetical | | | |
| | | | | Protein NOT Supported by | | | |
| | | | | cDNA | | | |
| 770 | Gma.4822.1.S1_at | 0.283 | BI969249 | Disease & Defense | | | |
| 771 | GmaAffx.32328.1.A1_at | 0.283 | BI968685 | No Homology to | | | |
| | | | | Known Proteins | | | |
| 772 | Gma.3963.1.S1_at | 0.283 | AW202007 | Unclassified - Hypothetical | | | Zn-finger protein |
| | | | | Protein NOT Supported by | | | |
| | | | | cDNA | | | |
| 773 | GmaAffx.56606.1.S1_at | 0.283 | BG507644 | Secondary Metabolism | | | Multicopper oxidases |
| 774 | GmaAffx.47288.1.S1_at | 0.282 | BU764979 | Unclassified - Hypothetical | | | GTPase Rab18, small |
| | | | | Protein Supported by | | | G protein superfamily |
| | | | | cDNA | | | |
| 775 | Gma.850.1.S1_at | 0.281 | BE822050 | Unclassified - Hypothetical | | | |
| | | | | Protein NOT Supported by | | | |
| | | | | cDNA | | | |
| 776 | Gma.5634.1.S1_s_at | 0.28 | BE806891 | Secondary Metabolism | | | Steroid reductase |
| 777 | Gtna.1332.1.S1_s_at | 0.279 | BI970666 | Secondary Metabolism | Ethylene | | 1-aminocyclopropane- |
| | | | | | Biosynthesis | | 1-carboxylate synthase, |
| | | | | | | | and related proteins |
| 778 | Gma.1068.1.S1_at | 0.279 | L19360.1 | Signal Transduction | Protein Kinase | | Serine/threonine protein kinase |
| 779 | Gma.2590.1.A1_s_at | 0.279 | CD406617 | Disease & Defense | | | |
| 780 | Gma.6047.1.S1_s_at | 0.278 | BQ611159 | Unclassified - Protein with | | | |
| | | | | Unknown Function | | | |
| 781 | Gma.1716.1.S1_at | 0.278 | AW156801 | Unclassified - Hypothetical | | | |
| | | | | Protein Supported by | | | |
| | | | | cDNA | | | |
| 782 | GmaAffx.50646.1.A1_at | 0.277 | AW310451 | Unclassified - Hypothetical | | | Molybdenum cofactor sulfurase |
| | | | | Protein Supported by | | | |
| | | | | cDNA | | | |
| 783 | Gma.3591.2.S1_at | 0.277 | BE473658 | Secondary Metabolism | Ethylene | | Iron/ascorbate family |
| | | | | | Biosynthesis | | oxidoreductases |
| 784 | GmaAffx.90652.1.S1_at | 0.276 | CF806377 | Unclassified - Hypothetical | | | Predicted hydrolase/acyltransferase |
| | | | | Protein Supported by | | | (alpha/beta hydrolase superfamily) |
| | | | | cDNA | | | |
| 785 | Gma.17015.1.S1_at | 0.275 | AW733363 | No Homology to | | | |
| | | | | Known Proteins | | | |
| 786 | GmaAffx.80830.1.A1_at | 0.275 | BU549591 | Unclassified - Hypothetical | | | |
| | | | | Protein Supported by | | | |
| | | | | cDNA | | | |
| 787 | Gma.7494.1.S1_at | 0.275 | BU548105 | Signal Transduction | Protein Phosphatase | | Serine/threonine protein phosphatase |
| 788 | GmaAffx.79516.1.A1_at | 0.275 | CD400596 | Transcription | Transcription | MYB | |
| | | | | | Factor | | |
| 789 | Gma.4593.1.S1_a_at | 0.275 | CD396298 | Energy | | | |
| 790 | Gma.8514.1.S1_at | 0.275 | CA851666 | Protein Destination & Storage | | | Glutathione S-transferase |
| 791 | GmaAffx.85528.1.S1_at | 0.274 | BE608070 | Unclassified - Hypothetical | | | FOG: PPR repeat |
| | | | | Protein Supported by | | | |
| | | | | cDNA | | | |
| 792 | Gma.6763.1.A1_at | 0.273 | BU544794 | Energy | | | Ferric reductase, NADH/NADPH |
| | | | | | | | oxidase and related proteins |
| 793 | Gma.6623.2.S1_s_at | 0.273 | BQ629284 | Unclassified - Protein with | | | 1,4-benzoquinonereductase-like; Trp |
| | | | | Unknown Function | | | repressor binding protein-like/ |
| | | | | | | | protoplast-secreted protein |
| 794 | GmaAffx.68507.1.S1_at | 0.273 | BI699405 | Transcription | Transcription | Heat Shock | Heat shock transcription factor |
| | | | | | Factor | | |
| 795 | GmaAffx.80393.1.A1_at | 0.272 | BE658680 | No Homology to | | | |
| | | | | Known Proteins | | | |
| 796 | GmaAffx.92568.1.S1_at | 0.272 | CF808293 | Metabolism | | | Acyl-CoA-binding protein |
| 797 | Gma.8022.1.S1_at | 0.272 | BQ628998 | Disease & Defense | | | Soluble epoxide hydrolase |
| 798 | Gma.9534.1.S1_at | 0.272 | CA802874 | Disease & Defense | | | |
| 799 | GmaAffx.36470.1.A1_at | 0.272 | BU550552 | Unclassified - Hypothetical | | | |
| | | | | Protein Supported by | | | |
| | | | | cDNA | | | |
| 800 | Gma.11284.1.S1_at | 0.272 | AW153035 | Disease & Defense | | | |
| 801 | Gma.6999.1.S1_x_at | 0.271 | AF529303.1 | Disease & Defense | | | |
| 802 | GmaAffx.64967.1.S1_at | 0.271 | BU546115 | Secondary Metabolism | | | Iron/ascorbate family |
| | | | | | | | oxidoreductases |
| 803 | Gma.6550.1.A1_at | 0.271 | CD402638 | Metabolism | | | Hydroxyindole-O-methyltransferase |
| | | | | | | | and related SAM-dependent |
| | | | | | | | methyltransferases |
| 804 | GmaAffx.15471.1.S1_at | 0.271 | BQ611747 | Transcription | Transcription | MYB | Transcription factor, Myb |
| | | | | | Factor | | superfamily |
| 805 | Gma.15155.2.S1_at | 0.27 | BG789619 | Unclassified - Hypothetical | | | |
| | | | | Protein Supported by | | | |
| | | | | cDNA | | | |
| 806 | Gma.426.1.A1_at | 0.27 | AI442768 | Transcription | Transcription | Zinc Finger | |
| | | | | | Factor | | |
| 807 | Gma.17300.1.A1_at | 0.27 | AW350946 | Unclassified - Hypothetical | | | |
| | | | | Protein NOT Supported by | | | |
| | | | | cDNA | | | |
| 808 | GmaAffx.25612.1.S1_at | 0.269 | AW317577 | No Homology to | | | |
| | | | | Known Proteins | | | |
| 809 | GmaAffx.33527.1.S1_at | 0.268 | BU761195 | No Homology to | | | |
| | | | | Known Proteins | | | |
| 810 | GmaAffx.11999.1.S1_at | 0.267 | AW567661 | No Homology to | | | |
| | | | | Known Proteins | | | |
| 811 | Gma.4093.2.S1_at | 0.267 | BQ081460 | Transporter | | | |
| 812 | Gma.11277.2.S1_at | 0.267 | BI701238 | Unclassified - Hypothetical | | | |
| | | | | Protein NOT Supported by | | | |
| | | | | cDNA | | | |
| 813 | Gma.398.1.A1_at | 0.267 | AW350679 | Metabolism | | | Homocysteine S-methyltransferase |
| 814 | GmaAffx.91266.1.S1_s_at | 0.267 | CF806991 | Unclassified - Hypothetical | | | |
| | | | | Protein Supported by | | | |
| | | | | cDNA | | | |
| 815 | GmaAffx.130I.59.S1_at | 0.266 | BI974591 | Intracellular Traffic | | | Annexin |
| 816 | Gma.1137.1.S1_a_at | 0.266 | BF067710 | Metabolism | | | |
| 817 | GmaAffx.45805.2.S1_at | 0.265 | CA784263 | Metabolism | | | Hydroxyindole-O-methyltransferase |
| | | | | | | | and related SAM-dependent |
| | | | | | | | methyltransferases |
| 818 | GmaAffx.84566.1.S1_at | 0.265 | BI786849 | Transcription | Transcription | MYB | Transcription factor, Myb |
| | | | | | Factor | | superfamily |
| 819 | GmaAffx.69198.1.S1_at | 0.264 | BM526951 | Unclassified - Hypothetical | | | Leucine rich repeat proteins, |
| | | | | Protein Supported by | | | some proteins contain F-box |
| | | | | cDNA | | | |
| 820 | GmaAffx.4888.1.A1_at | 0.264 | BI967635 | No Homology to | | | |
| | | | | Known Proteins | | | |
| 821 | Gma.14897.2.S1_at | 0.263 | BQ785154 | Unclassified - Protein with | | | Dihydroorotase and related enzymes |
| | | | | Unknown Function | | | |
| 822 | Gma.8515.1.S1_at | 0.263 | AF243372.1 | Protein Destination & Storage | | | Glutathione S-transferase |
| 823 | GmaAffx.53914.1.A1_at | 0.262 | BE822062 | No Homology to | | | |
| | | | | Known Proteins | | | |
| 824 | Gma.1758.1.S1_at | 0.262 | CD402046 | Unclassified - Protein with | | | |
| | | | | Unknown Function | | | |
| 825 | Gma.4470.1.S1_s_at | 0.262 | AW156251 | Unclassified - Protein with | | | |
| | | | | Unknown Function | | | |
| 826 | GmaAffx.86927.1.S1_at | 0.261 | CA937162 | No Homology to | | | |
| | | | | Known Proteins | | | |
| 827 | GmaAffx.60958.1.S1_at | 0.261 | BG551038 | Transcription | Transcription | Basic Helix- | |
| | | | | | Factor | Loop-Helix | |
| | | | | | | (bHLH) | |
| 828 | Gma.3893.3.S1_at | 0.261 | BG651854 | Unclassified - Hypothetical | | | |
| | | | | Protein Supported by | | | |
| | | | | cDNA | | | |
| 829 | Gma.7580.1.S1_at | 0.261 | BE057763 | Unclassified - Hypothetical | | | |
| | | | | Protein Supported by | | | |
| | | | | cDNA | | | |
| 830 | GmaAffx.32345.1.A1_s_at | 0.261 | BI970277 | Cell Structure | | | |
| 831 | GmaAffx.86247.1.S1_s_at | 0.26 | BG238674 | Unclassified - Hypothetical | | | |
| | | | | Protein Supported by | | | |
| | | | | cDNA | | | |
| 832 | Gma.15359.1.S1_a_at | 0.26 | BM271008 | Unclassified - Protein with | | | Predicted Ca2+-dependent |
| | | | | Unknown Function | | | phospholipid-binding protein |
| 833 | GmaAffx.12933.1.S1_at | 0.26 | AW395787 | Transcription | Transcription | CCAAT | CCAAT-binding factor, subunit B |
| | | | | | Factor | | (HAP2) |
| 834 | GmaAffx.1301.68.S1_s_at | 0.26 | BI974374 | Intracellular | | | Annexin |
| | | | | Traffic | | | |
| 835 | GmaAffx.84424.1.S1_at | 0.259 | AW311543 | Metabolism | | | Alcohol dehydrogenase, class V |
| 836 | Gma.5578.3.S1_a_at | 0.259 | AW310572 | Metabolism | Cytochrome P450 | CYP98A | Cytochrome P450 CYP2 |
| | | | | | | | subfamily |
| 837 | GmaAffx.76821.1.S1_at | 0.259 | BG238158 | Unclassified - Protein with | Hormone Induced | | |
| | | | | Unknown Function | | | |
| 838 | GmaAffx.91328.1.S1_s_at | 0.258 | CF807053 | Unclassified - Hypothetical | | | Leucine rich repeat proteins, |
| | | | | Protein Supported by | | | some proteins contain F-box |
| | | | | cDNA | | | |
| 839 | GmaAffx.32606.1.S1_at | 0.258 | BM270707 | Unclassified - Hypothetical | | | Aquaporin |
| | | | | Protein NOT Supported by | | | (major intrinsic protein family) |
| | | | | cDNA | | | |
| 840 | GmaAffx.40015.1.S1_at | 0.258 | BE609673 | No Homology to | | | |
| | | | | Known Proteins | | | |
| 841 | GmaAffx.50613.1.A1_at | 0.258 | BI967418 | No Homology to | | | |
| | | | | Known Proteins | | | |
| 842 | Gma.7102.1.S1_a_at | 0.258 | AW309275 | Unclassified - Hypothetical | | | Leucine rich repeat proteins, |
| | | | | Protein Supported by | | | some proteins contain F-box |
| | | | | cDNA | | | |
| 843 | GmaAffx.77203.1.S1_at | 0.258 | BI970687 | Unclassified - Hypothetical | | | |
| | | | | Protein Supported by | | | |
| | | | | cDNA | | | |
| 844 | Gma.3000.1.S1_at | 0.257 | BE657172 | No Homology to | | | |
| | | | | Known Proteins | | | |
| 845 | Gma.728.1.A1_at | 0.257 | BE658779 | No Homology to | | | |
| | | | | Known Proteins | | | |
| 846 | Gma.1951.2.S1_a_at | 0.256 | CA783485 | Protein Destination & Storage | | | Serine carboxypeptidases |
| 847 | GmaAffx.45003.1.S1_at | 0.256 | BI968048 | Unclassified - Hypothetical | | | Uncharacterized conserved protein |
| | | | | Protein Supported by | | | |
| | | | | cDNA | | | |
| 848 | GmaAffx.20108.1.S1_at | 0.256 | CA801220 | Unclassified - Hypothetical | | | |
| | | | | Protein Supported by | | | |
| | | | | cDNA | | | |
| 849 | Gma.5634.1.S1_at | 0.256 | BE806891 | Secondary | | | Steroid reductase |
| | | | | Metabolism | | | |
| 850 | Gma.17516.1.S1_at | 0.255 | CD396417 | Transporter | | | Ca2+/H+ antiporter VCX1 |
| | | | | | | | and related proteins |
| 851 | GmaAffx.92148.1.S1_s_at | 0.255 | CF807873 | Unclassified - Hypothetical | | | |
| | | | | Protein NOT Supported by | | | |
| | | | | cDNA | | | |
| 852 | Gma.2080.2.S1_a_at | 0.255 | BM520916 | Secondary Metabolism | | | |
| 853 | GmaAffx.92136.1.S1_s_at | 0.254 | CF807451 | Signal Transduction | Protein Kinase | | Mannosyltransferase |
| 854 | GmaAffx.37803.2.S1_at | 0.254 | BE347017 | No Homology to | | | |
| | | | | Known Proteins | | | |
| 855 | GmaAffx.53324.1.A1_at | 0.254 | AW309251 | Transcription | Transcription | Basic Helix- | |
| | | | | | Factor | Loop-Helix | |
| | | | | | | (bHLH) | |
| 856 | GmaAffx.19118.1.S1_at | 0.253 | BM523731 | Unclassified - Hypothetical | | | |
| | | | | Protein NOT Supported by | | | |
| | | | | cDNA | | | |
| 857 | GmaAffx.51466.1.A1_at | 0.253 | CD406245 | Metabolism | | | UDP-glucuronosyl and UDP- |
| | | | | | | | glucosyl transferase |
| 858 | Gma.2102.1.S1_s_at | 0.253 | BM523392 | Disease & Defense | | | |
| 859 | GmaAffx.83172.1.S1_at | 0.253 | BQ610576 | Unclassified - Protein with | | | |
| | | | | Unknown Function | | | |
| 860 | GmaAffx.90069.1.S1_s_at | 0.253 | CF805794 | Unclassified - Hypothetical | | | Leucine rich repeat proteins, |
| | | | | Protein Supported by | | | some proteins contain F-box |
| | | | | cDNA | | | |
| 861 | Gma.6730.1.S1_s_at | 0.252 | BE211440 | Metabolism | | | UDP-glucuronosyl and UDP- |
| | | | | | | | glucosyl transferase |
| 862 | GmaAffx.85662.1.S1_at | 0.252 | BG404902 | Unclassified - Protein with | | | Predicted membrane protein, |
| | | | | Unknown Function | | | contains DoH and Cytochrome b-561/ |
| | | | | | | | ferric reductase transmembrane domains |
| 863 | Gma.1619.1.S1_at | 0.252 | AW472195 | Unclassified - Hypothetical | | | |
| | | | | Protein Supported by | | | |
| | | | | cDNA | | | |
| 864 | Gma.1495.1.S1_at | 0.251 | BI973545 | No Homology to | | | |
| | | | | Known Proteins | | | |
| 865 | Gma.7836.1.A1_at | 0.251 | BU549650 | Unclassified - Hypothetical | | | |
| | | | | Protein NOT Supported by | | | |
| | | | | cDNA | | | |
| 866 | Gma.11277.1.S1_a_at | 0.251 | CA802310 | Protein Destination & Storage | | | |
| 867 | Gma.9086.2.S1_at | 0.251 | BE555169 | Cell Growth | | | |
| | | | | & Division | | | |
| 868 | GmaAffx.87547.2.S1_at | 0.251 | BQ080473 | Cell Structure | | | Beta-glucosidase, lactase |
| | | | | | | | phlorizinhydrolase, and related |
| | | | | | | | proteins |
| 869 | GmaAffx.47891.1.S1_s_at | 0.251 | BQ785880 | Cell Structure | | | |
| 870 | GmaAffx.50811.2.S1_at | 0.251 | BG725712 | Transcription | Transcription | NAC Domain | |
| | | | | | Factor | | |
| 871 | GmaAffx.72630.1.S1_at | 0.25 | BG239729 | Unclassified - Hypothetical | | | FOG: TPR repeat |
| | | | | Protein NOT Supported by | | | |
| | | | | cDNA | | | |
| 871 | GmaAffx.72630.1.S1_at | 0.25 | BG239729 | Unclassified - Hypothetical | | | Thioredoxin |
| | | | | Protein NOT Supported by | | | |
| | | | | cDNA | | | |
| 872 | Gma.8365.1.S1_at | 0.25 | CD396813 | Unclassified - | | | |
| | | | | Hypothetical | | | |
| | | | | Protein NOT | | | |
| | | | | Supported by | | | |
| | | | | cDNA | | | |
| 873 | Gma.9856.1.S1_at | 0.25 | BG650352 | Signal Transduction | Protein Kinase | | Serine/threonine protein kinase |
| 874 | Gma.8113.1.A1_at | 0.249 | BU548342 | No Homology to | | | |
| | | | | Known Proteins | | | |
| 875 | Gma.16875.1.A1_at | 0.248 | CD406723 | Metabolism | | | Aldo/keto reductase |
| | | | | | | | family proteins |
| 876 | Gma.4249.1.S1_at | 0.248 | BF324893 | Secondary | | | Methyltransferase |
| | | | | Metabolism | | | |
| 877 | GmaAffx.5743.1.S1_at | 0.247 | BQ628343 | Unclassified - Hypothetical | | | FOG: Predicted E3 |
| | | | | Protein Supported by | | | ubiquitin ligase |
| | | | | cDNA | | | |
| 878 | Gma.6719.2.S1_at | 0.247 | BG726773 | No Homology to | | | |
| | | | | Known Proteins | | | |
| 879 | Gma.358.1.S1_at | 0.247 | AW310270 | Metabolism | | | Arylacetamide deacetylase |
| 880 | Gma.1951.1.S1_at | 0.246 | BQ079657 | Protein Destination & Storage | | | Serine carboxypeptidases |
| 881 | GmaAffx.2026.1.A1_at | 0.246 | BU550139 | Transcription | Transcription | NAC Domain | |
| | | | | | Factor | | |
| 882 | Gma.10342.1.S1_at | 0.246 | BE058741 | Unclassified - Hypothetical | | | |
| | | | | Protein Supported by | | | |
| | | | | cDNA | | | |
| 883 | GmaAffx.82936.1.S1_at | 0.246 | AI938364 | Unclassified - Protein with | | | |
| | | | | Unknown Function | | | |
| 884 | GmaAffx.45957.2.S1_at | 0.246 | BI974135 | No Homology to | | | |
| | | | | Known Proteins | | | |
| 885 | GmaAffx.61872.1.S1_at | 0.245 | AW102386 | Signal Transduction | Protein Kinase | | |
| 886 | Gma.2439.1.S1_at | 0.244 | BI972053 | Metabolism | | | Predicted PhzC/PhzF-type epimerase |
| 887 | GmaAffx.24102.1.S1_at | 0.244 | BQ453911 | Unclassified - Hypothetical | | | |
| | | | | Protein Supported by | | | |
| | | | | cDNA | | | |
| 888 | GmaAffx.90395.1.A1_s_at | 0.244 | CF807499 | Unclassified - Protein with | | | |
| | | | | Unknown Function | | | |
| 889 | GmaAffx.15613.1.S1_at | 0.243 | AI939073 | Unclassified - Hypothetical | | | |
| | | | | Protein NOT Supported by | | | |
| | | | | cDNA | | | |
| 890 | GmaAffx.90331.1.S1_s_at | 0.243 | CF806056 | Disease & Defense | | | |
| 891 | GmaAffx.30421.1.S1_at | 0.243 | BU082092 | Transcription | Transcription | Zinc Finger | FOG: Predicted E3 |
| | | | | | Factor | | ubiquitin ligase |
| 892 | GmaAffx.22552.1.S1_at | 0.243 | BU549952 | Transporter | | | H+/oligopeptide symporter |
| 893 | GmaAffx.93301.2.S1_s_at | 0.242 | CF808497 | Secondary Metabolism | | | Flavonol reductase/cinnamoyl-CoA |
| | | | | | | | reductase |
| 894 | Gma.3531.1.S1_s_at | 0.241 | BI498803 | Unclassified - Hypothetical | | | FOG: PPR repeat |
| | | | | Protein Supported by | | | |
| | | | | cDNA | | | |
| 895 | Gma.4665.1.A1_at | 0.241 | AW309815 | No Homology to | | | |
| | | | | Known Proteins | | | |
| 896 | GmaAffx.24368.1.A1_at | 0.241 | BU545331 | No Homology to | | | |
| | | | | Known Proteins | | | |
| 897 | Gma.9086.1.S1_at | 0.24 | AW184932 | Cell Growth & Division | | | |
| 898 | Gma.3579.1.S1_at | 0.239 | AW349799 | Unclassified - Hypothetical | | | Kelch repeat-containing proteins |
| | | | | Protein Supported by | | | |
| | | | | cDNA | | | |
| 899 | Gma.5586.2.S1_at | 0.239 | CD398384 | Transcription | Transcription | Unclassified | CCAAT-binding factor, |
| | | | | | Factor | | subunit B (HAP2) |
| 900 | Gma.6280.2.S1_at | 0.239 | AI938985 | No Homology to | | | |
| | | | | Known Proteins | | | |
| 901 | GmaAffx.66451.1.S1_at | 0.238 | AW309198 | Secondary Metabolism | | | |
| 902 | Gma.418.1.A1_at | 0.237 | CA784607 | Unclassified - Hypothetical | | | Glycosyl transferase, family 8 - |
| | | | | Protein Supported by | | | glycogenin |
| | | | | cDNA | | | |
| 903 | GmaAffx.91479.1.S1_s_at | 0.237 | CF807204 | No Homology to | | | |
| | | | | Known Proteins | | | |
| 904 | Gma.13910.1.S1_at | 0.237 | CD409745 | Unclassified - Protein with | | | |
| | | | | Unknown Function | | | |
| 905 | Gma.17890.1.S1_at | 0.236 | AW432965 | No Homology to | | | |
| | | | | Known Proteins | | | |
| 906 | GmaAffx.89508.1.A1_s_at | 0.235 | CK606172 | Secondary Metabolism | | | Phenylalanine and histidine ammonia-lyase |
| 907 | Gma.10804.2.A1_s_at | 0.234 | CF921045 | Protein Destination & Storage | | | Molecular chaperone |
| | | | | | | | (DnaJ superfamily) |
| 908 | GmaAffx.52337.1.A1_at | 0.234 | CD413673 | No Homology to | | | |
| | | | | Known Proteins | | | |
| 909 | Gma.8988.1.S1_at | 0.234 | BU965547 | No Homology to | | | |
| | | | | Known Proteins | | | |
| 910 | GmaAffx.52391.1.S1_at | 0.233 | AW349563 | Unclassified - Hypothetical | | | |
| | | | | Protein NOT Supported by | | | |
| | | | | cDNA | | | |
| 911 | Gma.4837.1.S1_at | 0.232 | BI969343 | Transporter | | | H+/oligopeptide symporter |
| 912 | Gma.7788.1.A1_at | 0.232 | BU549148 | Unclassified - Hypothetical | | | Aspartyl protease |
| | | | | Protein Supported by | | | |
| | | | | cDNA | | | |
| 913 | GmaAffx.92230.1.A1_s_at | 0.23 | CF807955 | Disease & Defense | | | |
| 914 | Gma.13856.1.A1_at | 0.23 | CD408222 | No Homology to | | | |
| | | | | Known Proteins | | | |
| 915 | Gma.5294.1.S1_s_at | 0.23 | BE658252 | Unclassified - Hypothetical | | | |
| | | | | Protein Supported by | | | |
| | | | | cDNA | | | |
| 916 | GmaAffx.1242.1.A1_at | 0.229 | BU549839 | Metabolism | | | |
| 917 | Gma.15184.1.S1_at | 0.229 | BM522992 | Transcription | Transcription | Homeodomain | Transcriptionfactor HEX, |
| | | | | | Factor | | contains HOX and HALZ |
| | | | | | | | domains |
| 918 | GmaAffx.73554.1.S1_at | 0.228 | BM139469 | Unclassified - Hypothetical | | | |
| | | | | Protein NOT Supported by | | | |
| | | | | cDNA | | | |
| 919 | Gma.17326.1.A1_at | 0.228 | AW568254 | Unclassified - Hypothetical | | | Permease of the major |
| | | | | Protein NOT Supported by | | | facilitator superfamily |
| | | | | cDNA | | | |
| 920 | Gma.6599.1.S1_at | 0.228 | BQ629084 | Cell Structure | | | |
| 921 | GmaAffx.83818.1.S1_at | 0.227 | BU762716 | No Homology to | | | |
| | | | | Known Proteins | | | |
| 922 | GmaAffx.21041.1.A1_at | 0.227 | AW350819 | No Homology to | | | |
| | | | | Known Proteins | | | |
| 923 | GmaAffx.54085.2.S1_at | 0.226 | BQ629483 | No Homology to | | | |
| | | | | Known Proteins | | | |
| 924 | GmaAffx.88017.1.A1_at | 0.226 | BQ452554 | No Homology to | | | |
| | | | | Known Proteins | | | |
| 925 | GmaAffx.87793.2.S1_at | 0.225 | BQ740671 | Signal Transduction | Protein Kinase | | Glycogen synthase kinase-3 |
| 926 | Gma.16528.1.S1_at | 0.225 | CD398706 | Unclassified - Hypothetical | | | |
| | | | | Protein Supported by | | | |
| | | | | cDNA | | | |
| 927 | Gma.]0536.1.S1_at | 0.225 | BG508300 | No Homology to | | | |
| | | | | Known Proteins | | | |
| 928 | Gma.10830.1.S1_at | 0.225 | BE820822 | Unclassified - Hypothetical | | | |
| | | | | Protein Supported by | | | |
| | | | | cDNA | | | |
| 929 | GmaAffx.33121.1.S1_at | 0.224 | BQ611246 | Unclassified - Hypothetical | | | |
| | | | | Protein Supported by | | | |
| | | | | cDNA | | | |
| 930 | Gma.8441.1.S1_at | 0.223 | AY113187.2 | Secondary Metabolism | | | Multicopper oxidases |
| 931 | Gma.595.1.S1_at | 0.222 | AB004271.1 | Metabolism | | | |
| 932 | GmaAffx.19467.1.S1_at | 0.22 | BQ453237 | Signal Transduction | Protein Kinase | | Serine/threonine protein kinase |
| 933 | Gma.4981.1.A1_at | 0.22 | BI970256 | No Homology to | | | |
| | | | | Known Proteins | | | |
| 934 | GmaAffx.81585.1.S1_at | 0.22 | BM187887 | Unclassified - Hypothetical | | | Copper chaperone |
| | | | | Protein NOT Supported by | | | |
| | | | | cDNA | | | |
| 935 | Gma.6642.1.A1_at | 0.219 | BE610052 | Transcription | Transcription | Basic Leucine | FOG: Armadillo/beta- |
| | | | | | Factor | Zipper (bZIP) | catenin-like repeats |
| 936 | Gma.17688.1.S1_s_at | 0.219 | CA937360 | Transcription | Transcription | Basic Helix- | |
| | | | | | Factor | Loop-Helix | |
| | | | | | | (bHLH) | |
| 937 | GmaAffx.71222.1.S1_at | 0.218 | AW460050 | Metabolism | | | 5′-AMP-activated protein |
| | | | | | | | kinase, gamma subunit |
| 938 | GmaAffx.73943.1.S1_at | 0.218 | BM891115 | Unclassified - Hypothetical | | | |
| | | | | Protein Supported by | | | |
| | | | | cDNA | | | |
| 939 | GmaAffx.41230.1.A1_at | 0.218 | BI471609 | Unclassified - Hypothetical | | | |
| | | | | Protein NOT Supported by | | | |
| | | | | cDNA | | | |
| 940 | GmaAffx.92774.1.S1_at | 0.217 | CF808124 | Cell Structure | | | |
| 941 | Gma.3969.1.S1_s_at | 0.217 | AW349494 | Energy | | | |
| 942 | GmaAffx.21978.1.A1_at | 0.217 | CA803009 | No Homology to | | | |
| | | | | Known Proteins | | | |
| 943 | Gma.1195.1.S1_s_at | 0.217 | AI442801 | Metabolism | | | Predicted flavoprotein |
| 944 | GmaAffx.52114.1.S1_at | 0.216 | CA852143 | Metabolism | | | Heme oxygenase |
| 945 | GmaAffx.79390.3.S1_at | 0.216 | BU082743 | Transcription | Transcription | Basic Leucine | FOG: Armadillo/beta- |
| | | | | | Factor | Zipper (bZIP) | catenin-like repeats |
| 946 | GmaAffx.34453.1.S1_at | 0.215 | BU544114 | Unclassified - Hypothetical | | | |
| | | | | Protein Supported by | | | |
| | | | | cDNA | | | |
| 947 | Gma.13218.1.A1_s_at | 0.213 | CD391892 | No Homology to | | | |
| | | | | Known Proteins | | | |
| 948 | GmaAffx.61130.1.S1_at | 0.213 | CA799668 | Transporter | | | H+/oligopeptide symporter |
| 949 | GmaAffx.41078.1.S1_at | 0.213 | BI317149 | Metabolism | | | Alcohol dehydrogenase, class III |
| 950 | GmaAffx.49777.1.S1_at | 0.212 | BG047001 | No Homology to | | | |
| | | | | Known Proteins | | | |
| 951 | Gma.7254.1.S1_at | 0.212 | BU546166 | Unclassified - Hypothetical | | | |
| | | | | Protein Supported by | | | |
| | | | | cDNA | | | |
| 952 | GmaAffx.53256.1.S1_at | 0.211 | BU545086 | Unclassified - Hypothetical | | | |
| | | | | Protein Supported by | | | |
| | | | | cDNA | | | |
| 953 | Gma.11873.1.S1_at | 0.211 | CA819977 | Unclassified - Protein with | | | |
| | | | | Unknown Function | | | |
| 954 | Gma.2887.2.S1_x_at | 0.211 | BI701520 | Secondary | | | Phenylalanine and histidine ammonia- |
| | | | | Metabolism | | | lyase |
| 955 | Gma.2376.1.S1_at | 0.209 | CA852095 | Transcription | Transcription | Homeodomain | Transcription factor MEIS1 and |
| | | | | | Factor | | related HOX domain proteins |
| 956 | GmaAffx.15196.1.S1_at | 0.209 | BU549436 | Metabolism | | | Long-chain acyl-CoA synthetases |
| | | | | | | | (AMP-forming) |
| 957 | Gma.1490.1.S1_at | 0.209 | AB000097.1 | Cell Structure | | | Chitinase |
| 958 | Gma.3332.1.S1_at | 0.209 | BQ298780 | Protein Destination & Storage | | | |
| 959 | GmaAffx.14141.1.A1_at | 0.208 | BI973009 | No Homology to | | | |
| | | | | Known Proteins | | | |
| 960 | GmaAffx.88675.1.A1_at | 0.208 | CD417704 | Unclassified - Hypothetical | | | |
| | | | | Protein NOT Supported by | | | |
| | | | | cDNA | | | |
| 961 | Gma.18006.1.S1_at | 0.207 | CF922923 | Transcription | Transcription | Homeodomain | Transcription factor MEIS1 and |
| | | | | | Factor | | related HOX domain proteins |
| 962 | Gma.57.1.S1_at | 0.206 | AF532624.1 | Unclassified - Hypothetical | | | |
| | | | | Protein NOT Supported by | | | |
| | | | | cDNA | | | |
| 963 | Gma.5711.1.S1_at | 0.206 | BI784811 | Unclassified - Hypothetical | | | |
| | | | | Protein Supported by | | | |
| | | | | cDNA | | | |
| 964 | GmaAffx.56715.1.S1_at | 0.206 | BG041616 | Disease & Defense | | | |
| 965 | GmaAffx.42587.1.A1_at | 0.206 | BU544395 | No Homology to | | | |
| | | | | Known Proteins | | | |
| 966 | GmaAffx.91428.1.A1_at | 0.206 | CF807137 | Metabolism | | | |
| 967 | GmaAffx.8999.2.A1_s_at | 0.205 | BI700574 | No Homology to | | | |
| | | | | Known Proteins | | | |
| 968 | GmaAffx.12665.1.A1_at | 0.205 | BE822073 | Metabolism | | | |
| 969 | GmaAffx.43297.1.A1_at | 0.205 | AI442864 | Unclassified - Hypothetical | | | |
| | | | | Protein NOT Supported by | | | |
| | | | | cDNA | | | |
| 970 | Gma.15907.2.S1_at | 0.205 | BE329550 | Signal | Unclassified | | FOG: |
| | | | | Transduction | | | Leucine rich repeat |
| 971 | GmaAffx.35656.1.A1_at | 0.204 | BE822348 | No Homology to | | | |
| | | | | Known Proteins | | | |
| 972 | GmaAffx.62509.1.S1_s_at | 0.204 | BE022332 | Transcription | Transcription | MYB | Transcription factor, Myb |
| | | | | | Factor | | superfamily |
| 973 | Gma.4728.1.A1_at | 0.203 | BI968944 | Secondary Metabolism | | | NADH: flavin oxidoreductase/12- |
| | | | | | | | oxophytodienoate reductase |
| 974 | Gma.4746.1.A1_s_at | 0.203 | CD404836 | No Homology to | | | |
| | | | | Known Proteins | | | |
| 975 | GmaAffx.17918.1.S1_at | 0.203 | BG157024 | No Homology to | | | |
| | | | | Known Proteins | | | |
| 976 | Gma.149.1.S1_at | 0.203 | U43839.1 | Metabolism | | | Choline kinase |
| 977 | GmaAffx.48718.1.S1_at | 0.202 | BM528538 | No Homology to | | | |
| | | | | Known Proteins | | | |
| 978 | GmaAffx.64037.1.S1_at | 0.202 | BI321162 | Metabolism | Cytochrome P450 | CYP82F | Cytochrome P450 CYP2 |
| | | | | | | | subfamily |
| 979 | Gma.1929.1.S1_at | 0.201 | BU083650 | Transcription | Transcription | Homeodomain | Transcription factor HEX, |
| | | | | | Factor | | contains HOX and HALZ |
| | | | | | | | domains |
| 980 | Gma.2686.1.S1_s_at | 0.201 | BI970455 | Secondary Metabolism | | | |
| 981 | Gma.7549.1.S1_at | 0.201 | BG508637 | Unclassified - Protein with | | | |
| | | | | Unknown Function | | | |
| 982 | Gma.3987.2.S1_a_at | 0.2 | BI971594 | No Homology to | | | |
| | | | | Known Proteins | | | |
| 983 | GmaAffx.91641.1.S1_s_at | 0.2 | CF807366 | Disease & Defense | | | Acyl-CoA synthetase |
| 984 | Gma.3519.1.S1_at | 0.2 | CD403581 | Unclassified - Hypothetical | | | |
| | | | | Protein Supported by | | | |
| | | | | cDNA | | | |
| 985 | GmaAffx.11242.1.S1_s_at | 0.2 | AW317461 | Protein Destination & Storage | | | Serine carboxypeptidases |
| 986 | Gma.11522.2.S1_at | 0.199 | BG650872 | Unclassified - Hypothetical | | | |
| | | | | Protein Supported by | | | |
| | | | | cDNA | | | |
| 987 | Gma.16196.1.S1_at | 0.199 | AW350888 | Transcription | Transcription | Unclassified | |
| | | | | | Factor | | |
| 988 | GmaAffx.8712.1.S1_s_at | 0.199 | AW349107 | Disease & Defense | | | |
| 989 | GmaAffx.92649.1.S1_at | 0.198 | CF808374 | Metabolism | | | |
| 990 | Gma.7437.1.S1_at | 0.197 | BU547705 | Unclassified - Hypothetical | | | |
| | | | | Protein Supported by | | | |
| | | | | cDNA | | | |
| 991 | Gma.4099.1.S1_at | 0.196 | BG651846 | Metabolism | | | Beta-galactosidase |
| 992 | GmaAffx.32700.1.S1_at | 0.196 | BM522940 | No Homology to | | | |
| | | | | Known Proteins | | | |
| 993 | Gma.17662.2.S1_s_at | 0.195 | BQ630190 | Unclassified - Hypothetical | | | |
| | | | | Protein NOT Supported by | | | |
| | | | | cDNA | | | |
| 994 | GmaAffx.55657.1.S1_at | 0.193 | BM885600 | No Homology to | | | |
| | | | | Known Proteins | | | |
| 995 | Gma.3744.1.S1_at | 0.191 | CD417438 | Unclassified - Protein With | | | |
| | | | | Unknown Function | | | |
| 996 | Gma.8966.1.S1_at | 0.19 | BU765374 | Protein Destination & Storage | | | Predicted lipase |
| 997 | Gma.3118.1.S1_s_at | 0.189 | BQ786048 | Disease & Defense | | | Predicted metallothionein |
| 998 | Gma.6498.1.A1_at | 0.189 | BE824396 | Unclassified - Hypothetical | | | |
| | | | | Protein Supported by | | | |
| | | | | cDNA | | | |
| 999 | GmaAffx.6635.1.S1_at | 0.189 | CD412718 | Disease & Defense | | | |
| 1000 | GmaAffx.74323.1.S1_at | 0.188 | BI426587 | Unclassified - Hypothetical | | | Endoplasmic reticulum protein |
| | | | | Protein NOT Supported by | | | EP58, contains filamin rod |
| | | | | cDNA | | | domain and KDEL motif |
| 1001 | Gma.15625.1.S1_x_at | 0.188 | U77678.1 | Metabolism | Amino Acid | | Asparagine synthase |
| | | | | | Biosynthesis/ | | (glutamine-hydrolyzing) |
| | | | | | Metabolism | | |
| 1002 | GmaAffx.56301.1.S1_at | 0.188 | BQ610805 | No Homology to | | | |
| | | | | Known Proteins | | | |
| 1003 | GmaAffx.92693.1.S1_at | 0.186 | CF808418 | No Homology to | | | |
| | | | | Known Proteins | | | |
| 1004 | GmaAffx.23292.1.A1_at | 0.185 | BI967795 | No Homology to | | | |
| | | | | Known Proteins | | | |
| 1005 | GmaAffx.295.1.A1_s_at | 0.185 | BG653267 | Protein Destination & Storage | | | |
| 1006 | GmaAffx.27496.2.S1_at | 0.185 | BM523609 | Metabolism | | | |
| 1007 | Gma.12684.1.S1_at | 0.185 | CD394773 | Transcription | Transcription | Zinc Finger | |
| | | | | | Factor | | |
| 1008 | Gma.3744.2.S1_a_at | 0.185 | BI892693 | Unclassified - Protein With | | | |
| | | | | Unknown Function | | | |
| 1009 | Gma.10312.1.S1_at | 0.184 | AW706614 | Unclassified - Hypothetical | | | |
| | | | | Protein NOT Supported by | | | |
| | | | | cDNA | | | |
| 1010 | GmaAffx.81750.2.S1_at | 0.183 | BM568090 | Transcription | Transcription | Homeodomain | Transcription factor MEIS1 and |
| | | | | | Factor | | related HOX domain proteins |
| 1011 | GmaAffx.43809.1.S1_at | 0.183 | AW396565 | No Homology to | | | |
| | | | | Known Proteins | | | |
| 1012 | GmaAffx.68724.1.A1_at | 0.183 | BI970438 | No Homology to | | | |
| | | | | Known Proteins | | | |
| 1013 | Gma.3951.1.S1_at | 0.182 | BE659337 | Unclassified - Hypothetical | | | |
| | | | | Protein NOT Supported by | | | |
| | | | | cDNA | | | |
| 1014 | Gma.3165.1.S1_at | 0.181 | CD406755 | Transcription | Transcription | Homeodomain | Transcription factor HEX, |
| | | | | | Factor | | contains HOX and HALZ |
| | | | | | | | domains |
| 1015 | GmaAffx.14279.1.S1_at | 0.18 | BM887020 | Metabolism | | | |
| 1016 | Gma.6481.1.S1_at | 0.178 | CD416703 | Unclassified - Hypothetical | | | |
| | | | | Protein Supported by | | | |
| | | | | cDNA | | | |
| 1017 | Gma.15625.1.S1_a_at | 0.178 | U77678.1 | Metabolism | Amino Acid | | Asparagine synthase |
| | | | | | Biosynthesis/ | | (glutamine-hydrolyzing) |
| | | | | | Metabolism | | |
| 1018 | GmaAffx.70008.1.S1_at | 0.178 | BU762337 | Transcription | Transcription | MYB | Transcription factor, Myb |
| | | | | | Factor | | superfamily |
| 1019 | GmaAffx.26868.1.S1_at | 0.178 | BF325443 | Unclassified - Hypothetical | | | Tomosyn and related SNARE- |
| | | | | Protein NOT Supported by | | | interacting proteins |
| | | | | cDNA | | | |
| 1020 | GmaAffx.21924.1.S1_at | 0.177 | BQ611867 | Disease & Defense | | | |
| 1021 | Gma.9957.1.S1_at | 0.177 | BE820632 | Metabolism | | | UDP-glucuronosyl and UDP- |
| | | | | | | | glucosyl transferase |
| 1022 | Gma.17921.2.S1_s_at | 0.177 | BM085020 | Metabolism | Sucrose Biosynthesis | | Glycosyltransferase |
| 1023 | GmaAffx.40453.1.S1_at | 0.176 | BE658683 | No Homology to | | | |
| | | | | Known Proteins | | | |
| 1024 | GmaAffx.71842.1.S1_at | 0.176 | BE555317 | Metabolism | | | Possible pfkB family |
| | | | | | | | carbohydrate kinase |
| 1025 | GmaAffx.30792.1.A1_at | 0.176 | BU544815 | Unclassified - Hypothetical | | | |
| | | | | Protein Supported by | | | |
| | | | | cDNA | | | |
| 1026 | Gma.6192.1.S1_s_at | 0.175 | AW396773 | Metabolism | | | |
| 1027 | GmaAffx.46837.1.S1_at | 0.175 | BU550919 | Secondary Metabolism | | | Multicopper oxidases |
| 1028 | Gma.15534.1.S1_at | 0.175 | U50081.1 | Metabolism | | | |
| 1029 | GmaAffx.45036.1.S1_at | 0.175 | BF595470 | No Homology to | | | |
| | | | | Known Proteins | | | |
| 1030 | Gma.8490.1.S1_at | 0.175 | AB030492.1 | Metabolism | | | Protein involved in thiamine |
| | | | | | | | biosynthesis and DNA damage |
| | | | | | | | tolerance |
| 1031 | Gma.7422.1.S1_at | 0.174 | BQ612005 | Transporter | | | |
| 1032 | GmaAffx.79390.1.S1_at | 0.174 | BU550458 | Transcription | Transcription | Basic Leucine | FOG: Armadillo/beta- |
| | | | | | Factor | Zipper (bZIP) | catenin-like repeats |
| 1033 | Gma,1137.2.S1_x_at | 0.173 | CA852403 | Metabolism | | | |
| 1034 | Gma.6081.1.S1_at | 0.173 | BI967474 | Disease & Defense | | | Predicted chitinase |
| 1035 | GmaAffx.71308.1.S1_at | 0.173 | BF595112 | Transcription | Transcription | Heat Shock | Heat shock transcription factor |
| | | | | | Factor | | |
| 1036 | Gma.3522.1.S1_at | 0.173 | BM140209 | Unclassified - Hypothetical | | | |
| | | | | Protein Supported by | | | |
| | | | | cDNA | | | |
| 1037 | Gma.2801.1.S1_at | 0.173 | BI785739 | Cell Structure | | | Beta-glucosidase, lactase |
| | | | | | | | phlorizinhydrolase, |
| | | | | | | | and related proteins |
| 1038 | Gma.8370.1.S1_at | 0.172 | BQ454087 | Signal Transduction | Protein Phosphatase | | Serine/threonine protein phosphatase |
| 1039 | GmaAffx.89259.1.S1_s_at | 0.172 | CK605646 | Metabolism | | | |
| 1040 | GmaAffx.93436.1.A1_s_at | 0.17 | CF809161 | Transcription | Transcription | AP2/EREBP | |
| | | | | | Factor | | |
| 1041 | Gma.8704.1.S1_at | 0.17 | AW311022 | Unclassified - Hypothetical | | | FOG: TPR repeat |
| | | | | Protein NOT Supported by | | | |
| | | | | cDNA | | | |
| 1041 | Gma.8704.1.S1_at | 0.17 | AW311022 | Unclassified - Hypothetical | | | Thioredoxin |
| | | | | Protein NOT Supported by | | | |
| | | | | cDNA | | | |
| 1042 | GmaAffx.90677.1.S1_at | 0.17 | CF806402 | Unclassified - Hypothetical | | | DNA helicase PIF1/RRM3 |
| | | | | Protein NOT Supported by | | | |
| | | | | cDNA | | | |
| 1043 | GmaAffx.47230.1.S1_at | 0.169 | CA820051 | Transporter | | | Amino acid transporters |
| 1044 | Gma.16913.1.S1_s_at | 0.169 | BI426712 | Unclassified - Protein with | | | |
| | | | | Unknown Function | | | |
| 1045 | Gma.3000.2.S1_a_at | 0.168 | BM270844 | No Homology to | | | |
| | | | | Known Proteins | | | |
| 1046 | Gma.1394.1.S1_at | 0.167 | BG839300 | Metabolism | | | |
| 1047 | Gma.4120.2.S1_at | 0.167 | BI700246 | Unclassified - Protein with | | | |
| | | | | Unknown Function | | | |
| 1048 | Gma.3987.1.S1_at | 0.165 | BQ453408 | Unclassified - Hypothetical | | | Sodium sulfate symporter and |
| | | | | Protein NOT Supported by | | | related arsenite permeases |
| | | | | cDNA | | | |
| 1049 | GmaAffx.62509.2.S1_at | 0.164 | BE021475 | Transcription | Transcription | MYB | Transcription factor, Myb |
| | | | | | Factor | | superfamily |
| 1050 | GmaAffx.41917.1.S1_at | 0.164 | BE658926 | Disease & Defense | | | |
| 1051 | Gma.4095.1.S1_at | 0.164 | AW350037 | Unclassified - Hypothetical | | | |
| | | | | Protein Supported by | | | |
| | | | | cDNA | | | |
| 1052 | Gma.9036.1.S1_at | 0.163 | AW309501 | No Homology to | | | |
| | | | | Known Proteins | | | |
| 1053 | GmaAffx.55959.1.S1_at | 0.163 | BQ629429 | Unclassified - Hypothetical | | | |
| | | | | Protein NOT Supported by | | | |
| | | | | cDNA | | | |
| 1054 | Gma.4394.1.S1_a_at | 0.163 | CD409273 | Transcription | Transcription | Homeodomain | Transcription factor HEX, |
| | | | | | Factor | | contains HOX and HALZ domains |
| 1055 | Gma.3575.1.S1_at | 0.162 | BQ611599 | Cell Structure | | | |
| 1056 | Gma.1394.2.S1_at | 0.162 | BI470370 | Metabolism | | | |
| 1057 | Gma.5961.1.S1_at | 0.162 | AB052786.1 | Transporter | | | H+/oligopeptide symporter |
| 1058 | GmaAffx.39765.1.S1_s_at | 0.161 | BQ785586 | No Homology to | | | |
| | | | | Known Proteins | | | |
| 1059 | Gma.4479.1.S1_at | 0.161 | CA936529 | Unclassified - Hypothetical | | | |
| | | | | Protein Supported by | | | |
| | | | | cDNA | | | |
| 1060 | GmaAffx.89772.14.A1_s_at | 0.161 | CK606614 | Disease & Defense | | | |
| 1061 | Gma.10969.2.S1_at | 0.16 | CD402329 | Unclassified - Hypothetical | | | Predicted lipase |
| | | | | Protein NOT Supported by | | | |
| | | | | cDNA | | | |
| 1062 | GmaAffx.11815.1.S1_at | 0.16 | AW311637 | Metabolism | | | |
| 1063 | Gma.6192.1.S1_at | 0.159 | AW396773 | Metabolism | | | |
| 1064 | GmaAffx.77758.1.S1_x_at | 0.158 | BI944003 | No Homology to | | | |
| | | | | Known Proteins | | | |
| 1065 | Gma.12227.1.S1_at | 0.157 | BG511671 | Unclassified - Hypothetical | | | |
| | | | | Protein NOT Supported by | | | |
| | | | | cDNA | | | |
| 1066 | GmaAffx.15119.1.A1_at | 0.157 | BU544528 | Metabolism | | | Possible pfkB family |
| | | | | | | | carbohydrate kinase |
| 1067 | Gma.13150.1.A1_at | 0.157 | CD411577 | Unclassified - Protein with | Hormone Induced | | |
| | | | | Unknown Function | | | |
| 1068 | Gma.15996.1.S1_at | 0.156 | BQ454193 | Unclassified - Hypothetical | | | |
| | | | | Protein Supported by | | | |
| | | | | cDNA | | | |
| 1069 | GmaAffx.90812.1.S1_s_at | 0.155 | CF806537 | Disease & Defense | | | |
| 1070 | Gma.18006.1.A1_at | 0.155 | AI496486 | Transcription | Transcription | Homeodomain | Transcription factor MEIS1 and |
| | | | | | Factor | | related HOX domain proteins |
| 1071 | Gma.5863.2.S1_s_at | 0.155 | AW350762 | Secondary Metabolism | | | |
| 1072 | Gma.17739.1.S1_at | 0.155 | AW310535 | Unclassified - Hypothetical | | | |
| | | | | Protein Supported by | | | |
| | | | | cDNA | | | |
| 1073 | GmaAffx.81584.1.S1_at | 0.154 | BI426969 | Disease & Defense | | | |
| 1074 | GmaAffx.71308.2.A1_at | 0.153 | AW569888 | No Homology to | | | |
| | | | | Known Proteins | | | |
| 1075 | GmaAffx.5531.1.S1_at | 0.153 | BQ740711 | Cell Structure | | | |
| 1076 | Gma.10621.1.S1_at | 0.153 | AY004243.1 | Unclassified - Hypothetical | | | |
| | | | | Protein Supported by | | | |
| | | | | cDNA | | | |
| 1077 | Gma.14606.1.A1_at | 0.153 | BE821928 | Transporter | | | |
| 1078 | Gma.11028.1.S1_s_at | 0.152 | BE823084 | Metabolism | | | Glyoxylate/hydroxypyruvate |
| | | | | | | | reductase (D-isomer-specific 2-hydroxy |
| | | | | | | | acid dehydrogenase superfamily) |
| 1079 | Gma.8489.1.S1_s_at | 0.152 | AB030493.1 | Metabolism | | | Protein involved in thiamine |
| | | | | | | | biosynthesis and DNA damage tolerance |
| 1080 | Gma.4874.1.A1_at | 0.152 | AI938293 | No Homology to | | | |
| | | | | Known Proteins | | | |
| 1081 | Gma.5331.1.S1_at | 0.152 | BI892702 | Transcription | Transcription | NAC Domain | |
| | | | | | Factor | | |
| 1082 | GmaAffx.70005.1.S1_s_at | 0.15 | CA936335 | Secondary Metabolism | | | Iron/ascorbate family |
| | | | | | | | oxidoreductases |
| 1083 | GmaAffx.27331.1.S1_at | 0.15 | BM520297 | Unclassified - Hypothetical | | | |
| | | | | Protein NOT Supported by | | | |
| | | | | cDNA | | | |
| 1084 | Gma.13062.1.S1_at | 0.149 | CD394315 | Metabolism | | | |
| 1085 | Gma.4150.2.S1_s_at | 0.149 | AW350622 | Unclassified - Hypothetical | | | |
| | | | | Protein Supported by | | | |
| | | | | cDNA | | | |
| 1086 | Gma.6129.2.S1_s_at | 0.148 | AW186006 | Protein Synthesis | | | Translation initiation factor |
| | | | | | | | 5A (elF-5A) |
| 1087 | GmaAffx.11172.1.A1_at | 0.148 | CD404556 | No Homology to | | | |
| | | | | Known Proteins | | | |
| 1088 | GmaAffx.91555.1.A1_s_at | 0.148 | CF807280 | Metabolism | | | |
| 1089 | Gma.6999.3.S1_s_at | 0.147 | CF921432 | Disease & Defense | | | |
| 1090 | Gma.16842.1.A1_at | 0.147 | CD392652 | No Homology to | | | |
| | | | | Known Proteins | | | |
| 1091 | Gma.3604.1.S1_at | 0.147 | AW349604 | Cell Structure | | | O-methyltransferase |
| 1092 | GmaAffx.1785.1.S1_at | 0.146 | BF325331 | Transcription | Transcription | Homeodomain | Transcription factor HEX, |
| | | | | | Factor | | contains HOX and HALZ domains |
| 1093 | GmaAffx.93328.1.S1_s_at | 0.145 | CF809053 | Secondary Metabolism | | | |
| 1094 | GmaAffx.91205.1.S1_at | 0.144 | CF807869 | Metabolism | | | Glyoxylate/hydroxypyruvate |
| | | | | | | | reductase (D-isomer-specific 2-hydroxy |
| | | | | | | | acid dehydrogenase superfamily) |
| 1095 | GmaAffx.52963.1.S1_at | 0.144 | AW310673 | Unclassified - Protein with | | | |
| | | | | Unknown Function | | | |
| 1096 | GmaAffx.59470.1.S1_at | 0.143 | BI469872 | Secondary Metabolism | | | Phenylalanine and histidine |
| | | | | | | | ammonia-lyase |
| 1097 | GmaAffx.24992.1.S1_at | 0.143 | BU083490 | Protein Destination & Storage | | | Aspartyl protease |
| 1098 | GmaAffx.72700.1.S1_at | 0.143 | AW349825 | Unclassified - Hypothetical | | | |
| | | | | Protein NOT Supported by | | | |
| | | | | cDNA | | | |
| 1099 | Gma.11026.2.S1_s_at | 0.143 | BQ080041 | Disease & Defense | | | |
| 1100 | Gma.6643.1.A1_at | 0.143 | BE823205 | Secondary Metabolism | | | Sulfotransferase |
| 1101 | Gma.8318.1.A1_at | 0.143 | BU550469 | No Homology to | | | |
| | | | | Known Proteins | | | |
| 1102 | Gma.11026.1.S1_at | 0.143 | AW756869 | Disease & Defense | | | |
| 1103 | GmaAffx.4968.1.S1_at | 0.142 | BI967691 | Unclassified - Hypothetical | | | |
| | | | | Protein Supported by | | | |
| | | | | cDNA | | | |
| 1104 | GmaAffx.14279.2.A1_at | 0.142 | BM095037 | Metabolism | | | |
| 1105 | Gma.3799.2.S1_s_at | 0.142 | AW348702 | Unclassified - Protein with | | | |
| | | | | Unknown Function | | | |
| 1106 | GmaAffx.60479.1.S1_at | 0.141 | BG726023 | Transcription | Transcription | Homeodomain | Transcription factor ME1S1 and |
| | | | | | Factor | | related HOX domain proteins |
| 1107 | Gma.2842.1.S1_at | 0.141 | BG511652 | Unclassified - Protein with | | | Predicted |
| | | | | Unknown Function | | | Yippee-type zinc- |
| | | | | | | | binding protein |
| 1108 | GmaAffx.34657.1.A1_s_at | 0.14 | AW351012 | No Homology to | | | |
| | | | | Known Proteins | | | |
| 1109 | Gma.5760.2.S1_at | 0.14 | BQ298381 | Disease & Defense | | | |
| 1110 | GmaAffx.87433.1.S1_at | 0.14 | BI701509 | Unclassified - Hypothetical | | | |
| | | | | Protein Supported by | | | |
| | | | | cDNA | | | |
| 1111 | Gma.8187.1.S1_a_at | 0.139 | BU549133 | Signal Transduction | Protein Phosphatase | | Serine/threonine protein phosphatase |
| 1112 | Gma.17686.1.S1_at | 0.138 | BF597378 | Metabolism | Cytochrome P450 | CYP76C | Cytochrome P450 CYP2 |
| | | | | | | | subfamily |
| 1113 | Gma.6278.1.S1_at | 0.137 | BQ612647 | Unclassified - Hypothetical | | | |
| | | | | Protein Supported by | | | |
| | | | | cDNA | | | |
| 1114 | Gma.6643.2.S1_at | 0.137 | BE023121 | Secondary Metabolism | | | Sulfotransferase |
| 1115 | Gma.5867.1.A1_s_at | 0.136 | BI316163 | Metabolism | | | |
| 1116 | GmaAffx.81750.1.S1_at | 0.135 | AW306336 | No Homology to | | | |
| | | | | Known Proteins | | | |
| 1117 | GmaAffx.9944.1.S1_at | 0.135 | BM567882 | Cell Structure | | | |
| 1118 | Gma.5863.1.S1_at | 0.134 | AJ003245.1 | Secondary Metabolism | | | |
| 1119 | Gma.10969.1.S1_a_at | 0.133 | U36191.1 | Metabolism | | | |
| 1120 | GmaAffx.3711.1.A1_at | 0.133 | BE822116 | Unclassified - Hypothetical | | | |
| | | | | Protein NOT Supported by | | | |
| | | | | cDNA | | | |
| 1121 | GmaAffx.61593.1.S1_at | 0.133 | BU765478 | Unclassified - Hypothetical | | | Sodium sulfate symporter and |
| | | | | Protein NOT Supported by | | | related arsenite permeases |
| | | | | cDNA | | | |
| 1122 | Gma.5469.1.S1_at | 0.132 | AW349200 | No Homology to | | | |
| | | | | Known Proteins | | | |
| 1123 | Gma.17446.1.A1_at | 0.132 | AI461103 | Unclassified - Protein with | | | |
| | | | | Unknown Function | | | |
| 1124 | GmaAffx.17961.1.A1_at | 0.132 | BE657601 | Protein Destination & Storage | | | Molecular chaperones |
| | | | | | | | HSP70/HSC70, HSP70 superfamily |
| 1125 | Gma.3776.1.S1_at | 0.131 | AW780736 | Metabolism | | | Glutaredoxin and related |
| | | | | | | | proteins |
| 1126 | Gma.14471.1.S1_s_at | 0.131 | CF921241 | No Homology to | | | |
| | | | | Known Proteins | | | |
| 1127 | Gma.6129.2.S1_at | 0.131 | AW186006 | Protein Synthesis | | | Translation initiation factor |
| | | | | | | | 5A (elF-5A) |
| 1128 | GmaAffx.47806.1.A1_at | 0.13 | BG508458 | No Homology to | | | |
| | | | | Known Proteins | | | |
| 1129 | Gma.12094.1.A1_at | 0.13 | CD395088 | Unclassified - Hypothetical | | | |
| | | | | Protein Supported by | | | |
| | | | | cDNA | | | |
| 1130 | Gma.3578.1.S1_at | 0.129 | AW156522 | Disease & Defense | | | Acyl-CoA synthetase |
| 1131 | Gma.10896.3.A1_at | 0.129 | BI316837 | Metabolism | | | Aldehyde dehydrogenase |
| 1132 | GmaAffx.90677.1.S1_s_at | 0.128 | CF806402 | Unclassified - Hypothetical | | | DNA helicase PIF1/RRM3 |
| | | | | Protein NOT Supported by | | | |
| | | | | cDNA | | | |
| 1133 | Gma.10969.3.S1_x_at | 0.128 | CD396552 | Metabolism | | | |
| 1134 | Gma.4928.1.S1_a_at | 0.127 | AF002259.1 | Disease & Defense | | | Acyl-CoA synthetase |
| 1135 | GmaAffx.81362.1.S1_at | 0.126 | BF324988 | Cell Structure | | | |
| 1136 | GmaAffx.74256.1.S1_at | 0.126 | BE057293 | No Homology to | | | |
| | | | | Known Proteins | | | |
| 1137 | GmaAffx.91312.1.S1_s_at | 0.126 | CF806002 | Transcription | Transcription | Homeodomain | Transcription factor HEX, |
| | | | | | Factor | | contains HOX and HALZ domains |
| 1138 | Gma.2252.1.S1_at | 0.126 | CD406598 | Unclassified - Hypothetical | | | |
| | | | | Protein Supported by | | | |
| | | | | cDNA | | | |
| 1139 | GmaAffx.10204.1.S1_at | 0.126 | BQ298213 | Unclassified - Hypothetical | | | |
| | | | | Protein Supported by cDNA | | | |
| 1140 | Gma.3578.1.S1_s_at | 0.126 | AW156522 | Disease & Defense | | | Acyl-CoA synthetase |
| 1141 | Gma.1527.1.S1_x_at | 0.125 | BI968185 | Secondary Metabolism | | | Flavonol reductase/cinnamoyl-CoA |
| | | | | | | | reductase |
| 1142 | GmaAffx.93575.1.S1_s_at | 0.124 | CF808812 | No Homology to | | | |
| | | | | Known Proteins | | | |
| 1143 | GmaAffx.82218.1.S1_at | 0.124 | BI702052 | Secondary Metabolism | | | Iron/ascorbate family |
| | | | | | | | oxidoreductases |
| 1144 | Gma.4266.1.S1_at | 0.121 | BI972260 | No Homology to | | | |
| | | | | Known Proteins | | | |
| 1145 | Gma.9647.1.S1_at | 0.121 | BE820997 | Cell Structure | | | O-methyltransferase |
| 1146 | Gma.15.1.S1_at | 0.119 | U66316.1 | Unclassified - Protein with | | | |
| | | | | Unknown Function | | | |
| 1147 | Gma.6999.1.S1_s_at | 0.119 | AF529303.1 | Disease & Defense | | | |
| 1148 | GmaAffx.1301.78.S1_s_at | 0.119 | AW394497 | No Homology to | | | |
| | | | | Known Proteins | | | |
| 1149 | Gma,10827.3.S1_at | 0.119 | AW758966 | Secondary Metabolism | | | Iron/ascorbate family |
| | | | | | | | oxidoreductases |
| 1150 | Gma.3914.1.S1_at | 0.118 | BI702206 | Unclassified - Hypothetical | | | |
| | | | | Protein Supported by | | | |
| | | | | cDNA | | | |
| 1151 | Gma.17877.1.S1_at | 0.118 | BQ612471 | Unclassified - Protein with | | | |
| | | | | Unknown Function | | | |
| 1152 | Gma.320.1.S1_at | 0.117 | AF019116.1 | Disease & Defense | | | |
| 1153 | Gma.15523.1.S1_s_at | 0.116 | BE475550 | Cell Structure | | | |
| 1154 | Gma.16566.1.S1_at | 0.116 | CD400974 | Metabolism | | | Fumarase |
| 1155 | GmaAffx.90600.1.S1_at | 0.116 | CF806325 | Metabolism | | | |
| 1156 | Gma.2986.1.S1_at | 0.115 | BM271324 | Metabolism | | | 5'-AMP-activated protein |
| | | | | | | | kinase, gamma subunit |
| 1157 | GmaAffx.82000.1.S1_at | 0.115 | BI094895 | Unclassified - Hypothetical | | | |
| | | | | Protein Supported by | | | |
| | | | | cDNA | | | |
| 1158 | Gma.1269.1.S1_at | 0.115 | AW309671 | Metabolism | | | Flavonol reductase/cinnamoyl-CoA |
| | | | | | | | reductase |
| 1159 | Gma.14338.1.A1_at | 0.114 | CF923111 | Protein Destination & Storage | Storage Protein | | |
| 1160 | GmaAfix.73698.1.S1_at | 0.114 | BU964780 | Metabolism | | | 5′-AMP-activated protein |
| | | | | | | | kinase, gamma subunit |
| 1161 | GmaAffx.87793.1.S1_at | 0.114 | BI941782 | Unclassified - Hypothetical | | | |
| | | | | Protein NOT Supported by | | | |
| | | | | cDNA | | | |
| 1162 | Gma.6999.2.S1_s_at | 0.113 | X60043.1 | Disease & Defense | | | |
| 1163 | Gma.9638.1.A1_at | 0.113 | CA936403 | Disease & Defense | | | |
| 1164 | GmaAffx.34975.1.S1_at | 0.112 | BU547817 | Unclassified - Hypothetical | | | |
| | | | | Protein Supported by | | | |
| | | | | cDNA | | | |
| 1165 | GmaAffx.478.1.S1_at | 0.112 | BE822248 | Transcription | Transcription | Homeodomain | Transcription factor MEIS1 and |
| | | | | | Factor | | related HOX domain proteins |
| 1166 | Gma.2086.1.S1_at | 0.112 | AF035671.1 | Metabolism | Amino Acid | | Arginase family protein |
| | | | | | Biosynthesis/ | | |
| | | | | | Metabolism | | |
| 1167 | Gma.1661.1.S1_at | 0.111 | BE821382 | Metabolism | | | |
| 1168 | Gma.2133.1.S1_at | 0.11 | AW310071 | Unclassified - Protein with | | | |
| | | | | Unknown Function | | | |
| 1169 | GmaAffx.19541.1.S1_s_at | 0.109 | BQ612990 | No Homology to | | | |
| | | | | Known Proteins | | | |
| 1170 | GmaAffx.92927.1.S1_at | 0.109 | CF805992 | No Homology to | | | |
| | | | | Known Proteins | | | |
| 1171 | GmaAffx.87417.1.S1_at | 0.109 | BM568072 | Metabolism | | | |
| 1172 | GmaAffx.90600.1.S1_s_at | 0.108 | CF806325 | Metabolism | | | |
| 1173 | Gma.9740.1.S1_at | 0.108 | BQ081058 | Unclassified - Hypothetical | | | |
| | | | | Protein Supported by | | | |
| | | | | cDNA | | | |
| 1174 | Gma.15036.2.A1_at | 0.107 | CD411972 | Unclassified - Protein with | | | Predicted membrane protein |
| | | | | Unknown Function | | | |
| 1175 | Gma.4120.1.A1_at | 0.106 | AW310714 | Unclassified - Protein with | | | |
| | | | | Unknown Function | | | |
| 1176 | GmaAffx.487.1.S1_at | 0.106 | BG511495 | Transporter | | | Predicted transporter |
| | | | | | | | (major facilitator superfamily) |
| 1177 | Gma.10227.1.A1_at | 0.106 | BE020065 | Metabolism | | | Flavin-containing monooxygenase |
| 1178 | Gma.15568.1.S1_at | 0.103 | AI442650 | Disease & Defense | | | |
| 1179 | GmaAffx.30665.1.A1_at | 0.103 | BQ453743 | Signal Transduction | Protein Kinase | | Serine/threonine protein kinase |
| 1180 | GmaAffx.87670.1.S1_at | 0.103 | BQ452640 | Unclassified - Hypothetical | | | |
| | | | | Protein NOT Supported by | | | |
| | | | | cDNA | | | |
| 1181 | GmaAffx.76276.1.S1_at | 0.103 | BE331452 | Transporter | | | Copper chaperone |
| 1182 | Gma.4687.1.S1_at | 0.102 | AW309716 | Secondary Metabolism | | | |
| 1183 | GmaAffx.92479.1.S1_s_at | 0.101 | CF808204 | No Homology to | | | |
| | | | | Known Proteins | | | |
| 1184 | GmaAffx.34449.1.S1_at | 0.101 | AW164447 | No Homology to | | | |
| | | | | Known Proteins | | | |
| 1185 | Gma.2912.1.S1_s_at | 0.101 | AF117884.1 | Protein Destination & Storage | Storage Protein | | Uncharacterized conserved protein |
| 1186 | Gma.11028.2.S1_s_at | 0.101 | CA782711 | Metabolism | | | Glyoxylate/hydroxypyruvate |
| | | | | | | | reductase (D-isomer-specific 2-hydroxy |
| | | | | | | | acid dehydrogenase superfamily) |
| 1187 | GmaAffx.11897.1.S1_at | 0.1 | BE819868 | Transporter | | | Permease of the major facilitator |
| | | | | | | | superfamily |
| 1188 | Gma.11166.1.S1_s_at | 0.0999 | AW705829 | Metabolism | | | |
| 1189 | GmaAffx.9911.1.S1_at | 0.0948 | BU547808 | Unclassified - Hypothetical | | | Tomosyn and related SNARE- |
| | | | | Protein NOT Supported by | | | interacting proteins |
| | | | | cDNA | | | |
| 1190 | Gma.3774.2.S1_at | 0.0948 | CD393365 | Energy | | | Thioredoxin, nucleoredoxin and |
| | | | | | | | related proteins |
| 1191 | GmaAffx.83880.1.A1_at | 0.0946 | BE822640 | Disease & Defense | | | |
| 1192 | Gma.11166.1.S1_x_at | 0.0946 | AW705829 | Metabolism | | | |
| 1193 | Gma.1445.1.S1_at | 0.0927 | BQ454241 | Metabolism | | | Flavonol reductase/cinnamoyl-CoA |
| | | | | | | | reductase |
| 1194 | GmaAffx.26516.1.S1_at | 0.0916 | AW433266 | Cell Structure | | | |
| 1195 | GmaAffx.70435.2.S1_at | 0.0912 | AW705853 | Transcription | Transcription | Basic Helix- | |
| | | | | | Factor | Loop-Helix | |
| | | | | | | (bHLH) | |
| 1196 | Gma.13854.1.S1_at | 0.0911 | CD408166 | Metabolism | | | |
| 1197 | GmaAffx.93591.1.S1_s_at | 0.0905 | CF809316 | Metabolism | | | |
| 1198 | Gma.5622.1.S1_at | 0.0895 | BG363493 | No Homology to | | | |
| | | | | Known Proteins | | | |
| 1199 | GmaAffx.51464.1.S1_at | 0.089 | BQ628397 | No Homology to | | | |
| | | | | Known Proteins | | | |
| 1200 | GmaAffx.51444.1.S1_at | 0.0883 | BM178639 | Secondary Metabolism | | | Multicopper oxidases |
| 1201 | GmaAffx.74899.1.S1_at | 0.0855 | CA852377 | No Homology to | | | |
| | | | | Known Proteins | | | |
| 1202 | GmaAffx.24413.1.A1_at | 0.0849 | BU548368 | Metabolism | | | 5′-AMP-activated protein |
| | | | | | | | kinase, gamma subunit |
| 1203 | GmaAffx.3369.2.A1_at | 0.0845 | BU550316 | Cell Structure | | | |
| 1204 | Gma.17985.2.S1_at | 0.083 | BE802263 | Metabolism | | | |
| 1205 | Gma.15996.1.S1_x_at | 0.0827 | BQ454193 | Unclassified - Hypothetical | | | |
| | | | | Protein Supported by | | | |
| | | | | cDNA | | | |
| 1206 | Gma.9947.1.S1_at | 0.0812 | BF324269 | Unclassified - Hypothetical | | | |
| | | | | Protein Supported by | | | |
| | | | | cDNA | | | |
| 1207 | GmaAffx.90395.1.S1_s_at | 0.0789 | CF806120 | Unclassified - Protein with | | | |
| | | | | Unknown Function | | | |
| 1208 | Gma.2912.1.S1_at | 0.0785 | AF117884.1 | Protein Destination & Storage | Storage Protein | | Uncharacterized conserved protein |
| 1209 | Gma.4610.1.S1_s_at | 0.073 | BM522856 | No Homology to | | | |
| | | | | Known Proteins | | | |
| 1210 | Gma.79.1.S1_x_at | 0.0729 | S44202.1 | Unclassified - Protein with | | | |
| | | | | Unknown Function | | | |
| 1211 | Gma.1243.1.S1_at | 0.0725 | AW311027 | Unclassified - Hypothetical | | | |
| | | | | Protein Supported by | | | |
| | | | | cDNA | | | |
| 1212 | Gma.5947.1.S1_s_at | 0.0722 | BQ610509 | Disease & Defense | | | |
| 1213 | Gma.9010.1.S1_at | 0.0719 | CF921194 | Disease & Defense | | | |
| 1214 | Gma.16947.1.S1_at | 0.0696 | BG155375 | Unclassified - Hypothetical | | | |
| | | | | Protein Supported by | | | |
| | | | | cDNA | | | |
| 1215 | Gma.10216.1.S1_at | 0.0693 | BI969369 | Metabolism | | | Hydroxyindole-O-methyltransferase |
| | | | | | | | and related SAM-dependent |
| | | | | | | | methyltransferases |
| 1216 | GmaAffx.89407.1.A1_s_at | 0.0665 | CK606273 | Transporter | | | |
| 1217 | Gma.11211.1.S1_s_at | 0.0639 | AI442578 | No Homology to | | | |
| | | | | Known Proteins | | | |
| 1218 | Gma.17840.1.S1_at | 0.0614 | BE821995 | No Homology to | | | |
| | | | | Known Proteins | | | |
| 1219 | Gma.5611.1.S1_at | 0.0612 | AW309826 | Metabolism | Cytochrome P450 | CYP81D | Cytochrome P450 CYP2 |
| | | | | | | | subfamily |
| 1220 | Gma.79.1.S1_s_at | 0.0565 | S44202.1 | Unclassified - Protein with | | | |
| | | | | Unknown Function | | | |
| 1221 | Gma.4401.2.S1_a_at | 0.0559 | BI970725 | Disease & Defense | | | |
| 1222 | Gma.3774.1.S1_at | 0.0541 | BU550698 | Energy | | | Thioredoxin, nucleoredoxin and |
| | | | | | | | related proteins |
| 1223 | Gma.8425.1.S1_s_at | 0.0512 | D83968.1 | Metabolism | Cytochrome P450 | CYP93D | Cytochrome P450 CYP2 |
| | | | | | | | subfamily |
| 1224 | GmaAffx.10582.1.S1_at | 0.0508 | BM523687 | Transcription | Transcription | CCAAT | CCAAT-binding factor, subunit |
| | | | | | Factor | | A (HAP3) |
| 1225 | Gma.4796.1.A1_at | 0.0486 | AW102323 | Unclassified - Hypothetical | | | Uncharacterized conserved protein |
| | | | | Protein NOT Supported by | | | |
| | | | | cDNA | | | |
| 1226 | Gma.176.1.S1_s_at | 0.0411 | AF004810.1 | Protein Destination & Storage | Storage Protein | | |
| 1227 | Gma.15307.1.S1_at | 0.0401 | U64866.1 | Disease & Defense | | | |
| 1228 | Gma.3774.2.S1_a_at | 0.0399 | CD393365 | Energy | | | Thioredoxin, nucleoredoxin and |
| | | | | | | | related proteins |
| 1229 | Gma.1527.1.S1_a_at | 0.0379 | BI968185 | Secondary Metabolism | | | Flavonol reductase/cinnamoyl-CoA |
| | | | | | | | reductase |
| 1230 | GmaAffx.85382.1.S1_s_at | 0.0377 | BI945154 | No Homology to | | | |
| | | | | Known Proteins | | | |
| 1231 | Gma.1314.1.S1_at | 0.0357 | U63726.1 | Metabolism | | | Gamma-glutamyl hydrolase |
| 1232 | Gma.1527.2.S1_at | 0.029 | BE658017 | Secondary Metabolism | | | Flavonol reductase/cinnamoyl-CoA |
| | | | | | | | reductase |
| 1233 | GmaAffx.68621.1.A1_at | 0.0274 | BI893889 | Unclassified - Protein with | Stress Induced | | |
| | | | | Unknown Function | | | |
| 1234 | Gma.17985.1.S1_at | 0.0204 | CD416450 | Metabolism | | | |

TABLE 6

| | | Fold | | | | | |
|---|---|---|---|---|---|---|---|
| ID | Gene Name | Change | Soybean_Gen Bank_Acc | Functional Category | Sub-Family | Gene Family | Additional Description |
| 1 | Gma.10921.1.S1_s_at | 8.989 | BQ611250 | No Homology to Known | | | |
| | | | | Proteins | | | |
| 2 | Gma.12356.1.A1_at | 8.807 | CD397798 | Unclassified - Hypothetical | | | |
| | | | | Protein NOT Supported by | | | |
| | | | | cDNA | | | |
| 3 | Gma.5689.3.S1_s_at | 8.444 | BE657645 | Protein Destination & Storage | | | Gelatinase A and related matrix |
| | | | | | | | metalloproteases |
| 4 | Gma.964.1.A1_at | 5.456 | BE824146 | Metabolism | | | Tyrosine aminotransferase |
| 5 | Gma.4058.1.S1_at | 5.299 | AW310338 | Transporter | | | Predicted transporter (major facilitator |
| | | | | | | | superfamily) |
| 6 | Gma.17789.1.S1_at | 5.221 | BG511704 | Unclassified - Hypothetical | | | |
| | | | | Protein Supported by cDNA | | | |
| 7 | Gma.1221.1.S1_s_at | 5.045 | L23854.1 | Metabolism | | | Cytochrome b5 |
| 7 | Gma.1221.1.S1_s_at | 5.045 | L23854.1 | Metabolism | | | NADH-cytochrome b-5 reductase |
| 7 | Gma.1221.1.S1_s_at | 5.045 | L23854.1 | Metabolism | | | Sulfite oxidase, molybdopterin-binding |
| | | | | | | | component |
| 8 | GmaAffx.19116.1.S1_at | 4.956 | BI968419 | Signal Transduction | Protein | | |
| | | | | | Kinase | | |
| 9 | Gma.8416.1.S1_at | 3.823 | U13987.1 | Metabolism | | | Cytochrome b5 |
| 9 | Gma.8416.1.S1_at | 3.823 | U13987.1 | Metabolism | | | NADH-cytochrome b-5 reductase |
| 9 | Gma.8416.1.S1_at | 3.823 | U13987.1 | Metabolism | | | Sulfite oxidase, molybdopterin-binding |
| | | | | | | | component |
| 10 | Gma.6266.1.A1_at | 3.609 | CD413418 | Unclassified - Protein with | | | |
| | | | | Unknown Function | | | |
| 11 | Gma.4701.2.A1 a_at | 2.971 | BM526048 | Energy | | | Thioredoxin |
| 12 | GmaAffx.67477.2.S1_at | 2.756 | AW509184 | Cell Structure | | | |
| 13 | GmaAffx.53845.1.S1_at | 2.63 | BG406552 | Transporter | | | Sulfate/bicarbonate/oxalate exchanger |
| | | | | | | | SAT-1 and related transporters (SLC26 |
| | | | | | | | family) |
| 14 | GmaAffx.62542.1.S1_at | 2.266 | BE659136 | Unclassified - Hypothetical | | | |
| | | | | Protein NOT Supported by | | | |
| | | | | cDNA | | | |
| 15 | Gma.16521.1.S1_at | 2.185 | BE609300 | Unclassified - Hypothetical | | | Uncharacterized conserved protein |
| | | | | Protein Supported by cDNA | | | |
| 16 | Gma.4374.1.S1_at | 2.08 | BM178538 | Metabolism | Cytochrome | CYP76C | Cytochrome P450 CYP2 subfamily |
| | | | | | P450 | | |
| 17 | GmaAffx.6.1.S1_at | 2.07 | AW348562 | No Homology to Known | | | |
| | | | | Proteins | | | |
| 18 | Gma.2194.1.S1_at | 2.038 | BE657352 | Unclassified - Hypothetical | | | |
| | | | | Protein Supported by cDNA | | | |
| 19 | Gma.12641.1.A1_at | 0.5 | CD393495 | No Homology to Known | | | |
| | | | | Proteins | | | |
| 20 | GmaAffx.32345.1.A1_s_at | 0.499 | BI970277 | Cell Structure | | | |
| 21 | Gma.11665.2.A1_at | 0.498 | BU545959 | No Homology to Known | | | |
| | | | | Proteins | | | |
| 22 | GmaAffx.60958.1.S1_at | 0.494 | BG551038 | Transcription | Transcription | Basic Helix- | |
| | | | | | Factor | Loop-Helix | |
| | | | | | | (bHLH) | |
| 23 | GmaAffx.37484.1.S1_at | 0.494 | BG651852 | Unclassified - Protein with | | | Predicted membrane protein, contains |
| | | | | Unknown Function | | | DoH and Cytochrome b-561/ferric |
| | | | | | | | reductase transmembrane domains |
| 24 | GmaAffx.81585.1.S1_at | 0.493 | BM187887 | Unclassified - Hypothetical | | | Copper chaperone |
| | | | | Protein NOT Supported by | | | |
| | | | | cDNA | | | |
| 25 | GmaAffx.43774.1.A1_at | 0.492 | AW349173 | Cell Structure | | | |
| 26 | Gma.1704.1.S1_at | 0.492 | AW349971 | Metabolism | | | Glntaredoxin and related proteins |
| 27 | GmaAffx.72479.1.S1_at | 0.491 | BG043825 | Transcription | Transcription | AP2/EREBP | Uncharacterized conserved protein |
| | | | | | Factor | | |
| 28 | Gma.15907.2.S1_at | 0.491 | BE329550 | Signal Transduction | Unclassified | | FOG: Leucine rich repeat |
| 29 | Gma.13361.1.S1_at | 0.49 | AW309932 | Unclassified - Protein with | | | |
| | | | | Unknown Function | | | |
| 30 | Gma.11195.1.S1_at | 0.49 | CD417406 | Transporter | | | Apolipoprotein D/Lipocalin |
| 31 | Gma.4680.1.A1_s_at | 0.489 | AW309707 | Unclassified - Protein with | | | |
| | | | | Unknown Function | | | |
| 32 | GmaAffx.84795.1.S1_at | 0.489 | BG046191 | Unclassified - Hypothetical | | | |
| | | | | Protein NOT Supported by | | | |
| | | | | cDNA | | | |
| 33 | Gma.9013.1.S1_at | 0.488 | AF243368.1 | Disease & Defense | | | Glutathione S-transferase |
| 34 | Gma.8946.1.S1_at | 0.487 | BQ453813 | Transcription | Transcription | NAC | |
| | | | | | Factor | Domain | |
| 35 | GmaAffx.93189.1.S1_at | 0.487 | CF808914 | Metabolism | | | Carbon-nitrogen hydrolase |
| 36 | Gma.10896.3.A1_at | 0.486 | BI316837 | Metabolism | | | Aldehyde dehydrogenase |
| 37 | GmaAffx.83564.1.S1_at | 0.485 | BU578309 | No Homology to Known | | | |
| | | | | Proteins | | | |
| 38 | Gma.6129.2.S1_s_at | 0.484 | AW186006 | Protein Synthesis | | | Translation initiation factor 5A (eIF-5A) |
| 39 | Gma.1715.1.S1_at | 0.483 | BE191618 | Metabolism | | | |
| 40 | GmaAffx.72986.1.S1_at | 0.483 | BI317536 | Metabolism | | | Inositol polyphosphate 5-phosphatase |
| | | | | | | | and related proteins |
| 41 | GmaAffx.89049.1.S1_at | 0.483 | CK606168 | Intracellular Traffic | | | Annexin |
| 42 | GmaAffx.26967.1.S1_at | 0.482 | BE022958 | Unclassified - Hypothetical | | | |
| | | | | Protein Supported by cDNA | | | |
| 43 | Gma.2540.2.S1_s_at | 0.482 | BI787366 | No Homology to Known | | | |
| | | | | Proteins | | | |
| 44 | GmaAffx.28578.1.S1_at | 0.482 | BQ094224 | Disease & Defense | | | |
| 45 | GmaAffx.90331.1.S1_s_at | 0.482 | CF806056 | Disease & Defense | | | |
| 46 | Gma.5889.1.S1_at | 0.481 | BQ610202 | Unclassified - Hypothetical | | | Endoplasmic reticulum protein EP58, |
| | | | | Protein NOT Supported by | | | contains filamin rod domain and KDEL |
| | | | | cDNA | | | motif |
| 47 | Gma.1137.2.S1_x_at | 0.481 | CA852403 | Metabolism | | | |
| 48 | GmaAffx.28539.1.S1_at | 0.481 | BQ080184 | Disease & Defense | | | Ca2+-independent phospholipase A2 |
| 49 | GmaAffx.61311.1.S1_at | 0.48 | CA936951 | Transporter | | | Amino acid transporters |
| 50 | GmaAffx.30428.1.S1_at | 0.48 | BE824410 | Protein Destination & Storage | | | Molecular chaperones HSP70/HSC70, |
| | | | | | | | HSP70 superfamily |
| 51 | Gma.16585.1.S1_x_at | 0.48 | CA850597 | Metabolism | | | Predicted carbonic anhydrase involved |
| | | | | | | | in protection against oxidative damage |
| 52 | Gma.8160.1.S1_at | 0.48 | BI787214 | Unclassified - Hypothetical | | | |
| | | | | Protein NOT Supported by | | | |
| | | | | cDNA | | | |
| 53 | GmaAffx.87670.1.S1_at | 0.48 | BQ452640 | Unclassified - Hypothetical | | | |
| | | | | Protein NOT Supported by | | | |
| | | | | cDNA | | | |
| 54 | Gma.728.1.A1_at | 0.479 | BE658779 | No Homology to Known | | | |
| | | | | Proteins | | | |
| 55 | Gma.4120.1.A1_at | 0.479 | AW310714 | Unclassified - Protein with | | | |
| | | | | Unknown Function | | | |
| 56 | Gma.6630.1.S1_s_at | 0.478 | BG652851 | Unclassified - Hypothetical | | | |
| | | | | Protein Supported by cDNA | | | |
| 57 | GmaAffx.81584.1.S1_at | 0.478 | BI426969 | Disease & Defense | | | |
| 58 | GmaAffx.69076.1.S1_at | 0.477 | BM525610 | Unclassified - Hypothetical | | | |
| | | | | Protein NOT Supported by | | | |
| | | | | cDNA | | | |
| 59 | GmaAffx.53099.1.S1_at | 0.477 | BG043340 | Unclassified - Hypothetical | | | Alpha-L-fucosidase |
| | | | | Protein NOT Supported by | | | |
| | | | | cDNA | | | |
| 60 | Gma.8240.1.S1_at | 0.476 | BM092761 | Secondary Metabolism | | | Iron/ascorbate family oxidoreductases |
| 61 | GmaAffx.30665.1.A1_at | 0.476 | BQ453743 | Signal Transduction | Protein | | Serine/threonine protein kinase |
| | | | | | Kinase | | |
| 62 | GmaAffx.16207.1.S1_at | 0.475 | BE821188 | Cell Structure | | | Beta-glucosidase, lactase |
| | | | | | | | phlorizinhydrolase, and related proteins |
| 63 | GmaAffx.8712.1.S1_s_at | 0.474 | AW349107 | Disease & Defense | | | |
| 64 | Gma.1951.2.S1_a_at | 0.474 | CA783485 | Protein Destination & Storage | | | Serine carboxypeptidases |
| 65 | Gma.13000.1.S1_at | 0.474 | CD415111 | Unclassified - Hypothetical | | | |
| | | | | Protein Supported by cDNA | | | |
| 66 | Gma.9205.1.S1_at | 0.473 | BM528605 | Unclassified - Hypothetical | | | |
| | | | | Protein Supported by cDNA | | | |
| 67 | Gma.13035.1.S1_at | 0.473 | CD396536 | No Homology to Known | | | |
| | | | | Proteins | | | |
| 68 | Gma.10824.1.S1_at | 0.472 | BE824257 | Unclassified - Hypothetical | | | Magnesium transporters: CorA family |
| | | | | Protein Supported by cDNA | | | |
| 69 | GmaAffx.91641.1.S1_s_at | 0.472 | CF807366 | Disease & Defense | | | Acyl-CoA synthetase |
| 70 | Gma.4610.1.S1_s_at | 0.472 | BM522856 | No Homology to Known | | | |
| | | | | Proteins | | | |
| 71 | Gma.6498.1.A1_at | 0.471 | BE824396 | Unclassified - Hypothetical | | | |
| | | | | Protein Supported by cDNA | | | |
| 72 | GmaAffx.92136.1.S1_s_at | 0.471 | CF807451 | Signal Transduction | Protein | | Mannosyltransferase |
| | | | | | Kinase | | |
| 73 | GmaAffx.26320.1.S1_at | 0.469 | BE440416 | Unclassified - Hypothetical | | | |
| | | | | Protein Supported by cDNA | | | |
| 74 | Gma.1332.1.S1_s_at | 0.468 | BI970666 | Secondary Metabolism | Ethylene | | 1-aminocyclopropane-1-carboxylate |
| | | | | | Biosynthesis | | synthase, and related proteins |
| 75 | Gma.16477.1.S1_at | 0.467 | BE824134 | No Homology to Known | | | |
| | | | | Proteins | | | |
| 76 | GmaAffx.4936.1.S1_at | 0.467 | AW666270 | Transporter | | | Multidrug/pheromone exporter, ABC |
| | | | | | | | superfamily |
| 77 | Gma.7824.1.S1_at | 0.465 | BU550492 | Metabolism | | | Kynurenine 3-monooxygenase and |
| | | | | | | | related flavoprotein monooxygenases |
| 78 | Gma.3283.1.S1_at | 0.465 | BQ298733 | No Homology to Known | | | |
| | | | | Proteins | | | |
| 79 | GmaAffx.89049.1.S1_s_at | 0.464 | CK606168 | Intracellular Traffic | | | Annexin |
| 80 | Gma.16896.1.A1_at | 0.462 | CD416188 | No Homology to Known | | | |
| | | | | Proteins | | | |
| 81 | Gma.1980.1.A1_at | 0.462 | BM520466 | Signal Transduction | Protein | AtCIPKs | Serine/threonine protein kinase |
| | | | | | Kinase | | |
| 82 | GmaAffx.49130.1.S1_at | 0.462 | BE555035 | No Homology to Known | | | |
| | | | | Proteins | | | |
| 83 | GmaAffx.14141.1.A1_at | 0.462 | BI973009 | No Homology to Known | | | |
| | | | | Proteins | | | |
| 84 | Gma.10581.1.S1_at | 0.46 | AW396439 | Unclassified - Hypothetical | | | Predicted haloacid dehalogenase-like |
| | | | | Protein Supported by cDNA | | | hydrolase |
| 85 | GmaAffx.35729.1.S1_s_at | 0.46 | BE806047 | Unclassified - Hypothetical | | | Predicted E3 ubiquitin ligase |
| | | | | Protein Supported by cDNA | | | |
| 86 | Gma.412.1.S1_at | 0.459 | BE607835 | Protein Destination & Storage | | | Ubiquitin-protein ligase |
| 87 | GmaAffx.90069.1.S1_s_at | 0.459 | CF805794 | Unclassified - Hypothetical | | | Leucine rich repeat proteins, some |
| | | | | Protein Supported by cDNA | | | proteins contain F-box |
| 88 | Gma.17288.1.A1_at | 0.458 | AW310692 | No Homology to Known | | | |
| | | | | Proteins | | | |
| 89 | Gma.850.1.S1_at | 0.458 | BE822050 | Unclassified - Hypothetical | | | |
| | | | | Protein NOT Supported by | | | |
| | | | | cDNA | | | |
| 90 | GmaAffx.91731.1.S1_s_at | 0.458 | CF807456 | Secondary Metabolism | Ethylene | | Iron/ascorbate family oxidoreductases |
| | | | | | Biosynthesis | | |
| 91 | GmaAffx.74868.1.S1_at | 0.457 | CA820573 | No Homology to Known | | | |
| | | | | Proteins | | | |
| 92 | GmaAffx.29254.1.S1_at | 0.456 | AW76036I | Transcription | Transcription | Basic Helix- | |
| | | | | | Factor | Loop-Helix | |
| | | | | | | (bHLH) | |
| 93 | Gma.4394.1.S1_a_at | 0.456 | CD409273 | Transcription | Transcription | Homeodomain | Transcription factor HEX, contains |
| | | | | | Factor | | HOX and HALZ domains |
| 94 | Gma.3591.1.S1_a_at | 0.455 | BE440266 | Secondary Metabolism | Ethylene | | Iron/ascorbate family oxidoreductases |
| | | | | | Biosynthesis | | |
| 95 | Gma.1573.2.S1_a_at | 0.454 | BI974098 | Metabolism | | | |
| 96 | GmaAffx.90789.1.S1_s_at | 0.454 | CF8065I4 | Unclassified - Protein with | | | |
| | | | | Unknown Function | | | |
| 97 | GmaAffx.51444.1.S1_at | 0.453 | BM178639 | Secondary Metabolism | | | Multicopper oxidases |
| 98 | Gma.1068.1.S1_at | 0.453 | L19360.1 | Signal Transduction | Protein | | Serine/threonine protein kinase |
| | | | | | Kinase | | |
| 99 | GmaAffx.44471.1.A1_at | 0.452 | CA851833 | Unclassified - Hypothetical | | | |
| | | | | Protein NOT Supported by | | | |
| | | | | cDNA | | | |
| 100 | Gma.3531.1.S1_s_at | 0.452 | BI498803 | Unclassified - Hypothetical | | | FOG: PPR repeat |
| | | | | Protein Supported by cDNA | | | |
| 10I | Gma.16940.I.S1_at | 0.451 | AW310039 | Transcription | Transcription | Homeodomain | Transcription factor MEISI and related |
| | | | | | Factor | | HOX domain proteins |
| 102 | GmaAffx.32011.I.S1_at | 0.449 | BI701198 | No Homology to Known | | | |
| | | | | Proteins | | | |
| 103 | Gma.3963.2.S1_at | 0.449 | BE608192 | Unclassified - Hypothetical | | | Zn-finger protein |
| | | | | Protein NOT Supported by | | | |
| | | | | cDNA | | | |
| 104 | Gma.1263.1.S1_at | 0.449 | AY144180.1 | Metabolism | | | Ca2+/calmodulin-dependent protein |
| | | | | | | | kinase, EF-Hand protein superfamily |
| 105 | GmaAffx.72346.1.S1_at | 0.448 | BF424359 | Unclassified - Hypothetical | | | Speckle-type POZ protein SPOP and |
| | | | | Protein NOT Supported by | | | related proteins with TRAF, MATH and |
| | | | | cDNA | | | BTB/POZ domains |
| 106 | GmaAffx.82263.1.S1_s_at | 0.447 | AW568454 | No Homology to Known | | | |
| | | | | Proteins | | | |
| 107 | GmaAffx.83880.1.A1_at | 0.446 | BE822640 | Disease & Defense | | | |
| 108 | Gma.3893.3.S1_at | 0.446 | BG651854 | Unclassified - Hypothetical | | | |
| | | | | Protein Supported by cDNA | | | |
| 109 | GmaAffx.92184.1.S1_s_at | 0.445 | CF807909 | Unclassified - Protein with | | | |
| | | | | Unknown Function | | | |
| 110 | GmaAffx.45957.2.S1_at | 0.445 | BI974135 | No Homology to Known | | | |
| | | | | Proteins | | | |
| 111 | Gma.6264.1.S1_at | 0.444 | BQ612558 | Unclassified - Hypothetical | | | |
| | | | | Protein Supported by cDNA | | | |
| 112 | GmaAffx.93182.1.S1_s_at | 0.443 | CF808907 | Secondary Metabolism | Ethylene | | Iron/ascorbate family oxidoreductases |
| | | | | | Biosynthesis | | |
| 113 | GmaAffx.58073.1.S1_at | 0.443 | CA852105 | Unclassified - Hypothetical | | | |
| | | | | Protein Supported by cDNA | | | |
| 114 | Gma.11028.1.S1_s_at | 0.443 | BE823084 | Metabolism | | | Glyoxylate/hydroxypyruvate reductase |
| | | | | | | | (D-isomer-specific 2-hydroxy acid |
| | | | | | | | dehydrogenase superfamily) |
| 115 | Gma.6550.2.S1_s_at | 0.442 | BM086044 | Metabolism | | | Hydroxyindole-O-methyltransferase and |
| | | | | | | | related SAM-dependent |
| | | | | | | | methyltransferases |
| 116 | GmaAffx.91266.1.S1_s_at | 0.442 | CF806991 | Unclassified - Hypothetical | | | |
| | | | | Protein Supported by cDNA | | | |
| 117 | Gma.5331.1.S1_a_at | 0.442 | BI892702 | Transcription | Transcription | NAC | |
| | | | | | Factor | Domain | |
| 118 | Gma.3579.1.S1_at | 0.442 | AW349799 | Unclassified - Hypothetical | | | Kelch repeat-containing proteins |
| | | | | Protein Supported by cDNA | | | |
| 119 | Gma.1895.1.A1_at | 0.44 | AW309584 | Protein Destination & Storage | | | ER lumen protein retaining receptor |
| 120 | Gma.16528.1.S1_at | 0.44 | CD398706 | Unclassified - Hypothetical | | | |
| | | | | Protein Supported by cDNA | | | |
| 121 | Gma.10950.1.S1_at | 0.44 | BI699798 | Unclassified - Hypothetical | | | |
| | | | | Protein Supported by cDNA | | | |
| 122 | Gma.15996.1.S1_at | 0.439 | BQ454193 | Unclassified - Hypothetical | | | |
| | | | | Protein Supported by cDNA | | | |
| 123 | GmaAffx.1785.1.S1_at | 0.439 | BF325331 | Transcription | Transcription | Homeodomain | Transcription factor HEX. contains |
| | | | | | Factor | | HOX and HALZ domains |
| 124 | GmaAffx.93553.1.S1_s_at | 0.439 | CF809278 | Metabolism | | | |
| 125 | GmaAffx.74194.1.S1_at | 0.438 | BI968058 | Metabolism | | | 17 beta-hydroxysteroid dehydrogenase |
| | | | | | | | type 3. HSD17B3 |
| 126 | GmaAffx.87793.2.S1_at | 0.437 | BQ740671 | Signal Transduction | Protein | | Glycogen synthase kinase-3 |
| | | | | | Kinase | | |
| 127 | GmaAffx.36470.1.A1_at | 0.436 | BU550552 | Unclassified - Hypothetical | | | |
| | | | | Protein Supported by cDNA | | | |
| 128 | GmaAffx.42856.1.S1_at | 0.435 | BM567981 | Transcription | Transcription | Basic Helix- | |
| | | | | | Factor | Loop-Helix | |
| | | | | | | (bHLH) | |
| 129 | GmaAffx.1301.78.S1_s_at | 0.435 | AW394497 | No Homology to Known | | | |
| | | | | Proteins | | | |
| 130 | Gma.11334.1.S1_a_at | 0.433 | BI967327 | Cell Structure | | | O-methyltransferase |
| 131 | GmaAffx.92230.1.A1_s_at | 0.432 | CF807955 | Disease & Defense | | | |
| 132 | Gma.398.1.A1_at | 0.43 | AW350679 | Metabolism | | | Homocysteine S-methyltransferase |
| 133 | Gma.4665.1.A1_at | 0.43 | AW309815 | No Homology to Known | | | |
| | | | | Proteins | | | |
| 134 | GmaAffx.27331.1.S1_at | 0.428 | BM520297 | Unclassified - Hypothetical | | | |
| | | | | Protein NOT Supported by | | | |
| | | | | cDNA | | | |
| 135 | GmaAffx.34975.1.S1_at | 0.428 | BU547817 | Unclassified - Hypothetical | | | |
| | | | | Protein Supported by cDNA | | | |
| 136 | Gma.10969.1.S1_a_at | 0.426 | U36191.1 | Metabolism | | | |
| 137 | Gma.1661.1.S1_at | 0.424 | BE821382 | Metabolism | | | |
| 138 | Gma.2080.2.S1_a_at | 0.424 | BM520916 | Secondary Metabolism | | | |
| 139 | GmaAffx.25612.1.S1_at | 0.424 | AW317577 | No Homology to Known | | | |
| | | | | Proteins | | | |
| 140 | GmaAffx.50677.1.S1_s_at | 0.423 | BI971744 | No Homology to Known | | | |
| | | | | Proteins | | | |
| 141 | GmaAffx.89624.1.A1_s_at | 0.422 | CK606100 | Transcription | Transcription | Basic | |
| | | | | | Factor | Leucine | |
| | | | | | | Zipper | |
| | | | | | | (bZIP) | |
| 142 | Gma.8514.1.S1_at | 0.422 | CA851666 | Protein Destination & Storage | | | Glutathione S-transferase |
| 143 | GmaAffx.91328.1.S1_at | 0.421 | CF807053 | Unclassified - Hypothetical | | | Leucine rich repeat proteins, some |
| | | | | Protein Supported by cDNA | | | proteins contain F-box |
| 144 | GmaAffx.47891.1.S1_s_at | 0.42 | BQ785880 | Cell Structure | | | |
| 145 | Gma.6278.1.S1_at | 0.419 | BQ612647 | Unclassified - Hypothetical | | | |
| | | | | Protein Supported by cDNA | | | |
| 146 | GmaAffx.487.1.S1_at | 0.419 | BG511495 | Transporter | | | Predicted transporter (major facilitator |
| | | | | | | | superfamily) |
| 147 | Gma.17985.2.S1_at | 0.417 | BE802263 | Metabolism | | | |
| 148 | Gma.11277.2.S1_at | 0.416 | BI701238 | Unclassified - Hypothetical | | | |
| | | | | Protein NOT Supported by | | | |
| | | | | cDNA | | | |
| 149 | Gma.5400.1.S1_at | 0.416 | BM093775 | No Homology to Known | | | |
| | | | | Proteins | | | |
| 150 | GmaAffx.33121.1.S1_at | 0.416 | BQ611246 | Unclassified - Hypothetical | | | |
| | | | | Protein Supported by cDNA | | | |
| 151 | Gma.6550.2.S1_x_at | 0.413 | BM086044 | Metabolism | | | Hydroxyindole-O-methyltransferase and |
| | | | | | | | related SAM-dependent |
| | | | | | | | methyltransferases |
| 152 | GmaAffx.15222.1.S1_at | 0.413 | BU550700 | Unclassified - Hypothetical | | | |
| | | | | Protein NOT Supported by | | | |
| | | | | cDNA | | | |
| 153 | Gma.5586.2.S1_at | 0.412 | CD398384 | Transcription | Transcription | Unclassified | CCAAT-binding factor, subunit B |
| | | | | | Factor | | (HAP2) |
| 154 | Gma.10536.1.S1_at | 0.411 | BG508300 | No Homology to Known | | | |
| | | | | Proteins | | | |
| 155 | GmaAffx.74160.1.S1_at | 0.411 | BQ740845 | Protein Destination & Storage | | | |
| 156 | GmaAffx.11049.1.S1_at | 0.41 | BU545140 | Unclassified - Protein with | | | Phosphatidylethanolamine binding |
| | | | | Unknown Function | | | protein |
| 157 | Gma.17326.1.A1_at | 0.41 | AW568254 | Unclassified - Hypothetical | | | Permease of the major facilitator |
| | | | | Protein NOT Supported by | | | superfamily |
| | | | | cDNA | | | |
| 158 | Gma.16196.1.S1_at | 0.41 | AW350888 | Transcription | Transcription | Unclassified | |
| | | | | | Factor | | |
| 159 | GmaAffx.23289.1.S1_at | 0.409 | CD392087 | No Homology to Known | | | |
| | | | | Proteins | | | |
| 160 | GmaAffx.93592.1.S1_x_at | 0.409 | CF809317 | Unclassified - Hypothetical | | | |
| | | | | Protein Supported by cDNA | | | |
| 161 | GmaAffx.92774.1.S1_at | 0.408 | CF808124 | Cell Structure | | | |
| 162 | GmaAffx.81851.1.S1_at | 0.408 | AW706751 | Unclassified - Hypothetical | | | |
| | | | | Protein Supported by cDNA | | | |
| 163 | GmaAffx.92927.1.S1_at | 0.407 | CF805992 | No Homology to Known | | | |
| | | | | Proteins | | | |
| 164 | GmaAffx.64794.1.S1_at | 0.407 | CA801749 | Unclassified - Hypothetical | | | |
| | | | | Protein Supported by cDNA | | | |
| 165 | GmaAffx.478.2.S1_at | 0.406 | BU763963 | Transcription | Transcription | Homeodomain | Transcription factor ME1S1 and related |
| | | | | | Factor | | HOX domain proteins |
| 166 | GmaAffx.24368.1.A1_at | 0.405 | BU545331 | No Homology to Known | | | |
| | | | | Proteins | | | |
| 167 | Gma.1394.2.S1_a_at | 0.404 | BI470370 | Metabolism | | | |
| 168 | GmaAffx.84424.1.S1_at | 0.404 | AW311543 | Metabolism | | | Alcohol dehydrogenase, class V |
| 169 | GmaAffx.34449.1.S1_at | 0.403 | AW164447 | No Homology to Known | | | |
| | | | | Proteins | | | |
| 170 | GmaAffx.64037.1.S1_at | 0.403 | BI321162 | Metabolism | Cytochrome | CYP82F | Cytochrome P450 CYP2 subfamily |
| | | | | | P450 | | |
| 171 | Gma.6709.1.S1_at | 0.402 | BE823291 | Signal Transduction | Protein | AtClPKs | Serine/threonine protein kinase |
| | | | | | Kinase | | |
| 172 | Gma.17015.1.S1_at | 0.402 | AW733363 | No Homology to Known | | | |
| | | | | Proteins | | | |
| 173 | Gma.3963.1.S1_at | 0.402 | AW202007 | Unclassified - Hypothetical | | | Zn-finger protein |
| | | | | Protein NOT Supported by | | | |
| | | | | cDNA | | | |
| 174 | Gma.8113.1.A1_at | 0.401 | BU548342 | No Homology to Known | | | |
| | | | | Proteins | | | |
| 175 | GmaAffx.88675.1.A1_at | 0.4 | CD417704 | Unclassified - Hypothetical | | | |
| | | | | Protein NOT Supported by | | | |
| | | | | cDNA | | | |
| 176 | GmaAffx.5255.1.S1_at | 0.398 | BG362929 | Transcription | Transcription | AP2/EREBP | |
| | | | | | Factor | | |
| 177 | Gma.5294.1.S1_s_at | 0.398 | BE658252 | Unclassified - Hypothetical | | | |
| | | | | Protein Supported by cDNA | | | |
| 178 | Gma.3776.1.S1_at | 0.398 | AW780736 | Metabolism | | | Glutaredoxin and related proteins |
| 179 | GmaAffx.80393.1.A1_at | 0.397 | BE658680 | No Homology to Known | | | |
| | | | | Proteins | | | |
| 180 | GmaAffx.82936.1.S1_at | 0.397 | AI938364 | Unclassified - Protein with | | | |
| | | | | Unknown Function | | | |
| 181 | Gma.6075.1.S1_a_at | 0.396 | AW317183 | Disease & Defense | | | |
| 182 | GmaAffx.62364.1.S1_at | 0.395 | AW733764 | Protein Destination & Storage | | | Dipeptidyl aminopeptidase |
| 183 | GmaAffx.79390.3.S1_at | 0.393 | BU082743 | Transcription | Transcription | Basic | FOG: Armadillo/beta-catenin-like |
| | | | | | Factor | Leucine | repeats |
| | | | | | | Zipper | |
| | | | | | | (bZIP) | |
| 184 | GmaAffx.4888.1.A1_at | 0.392 | BI967635 | No Homology to Known | | | |
| | | | | Proteins | | | |
| 185 | GmaAffx.79390.1.S1_at | 0.39 | BU550458 | Transcription | Transcription | Basic | FOG: Armadillo/beta-catenin-like |
| | | | | | Factor | Leucine | repeats |
| | | | | | | Zipper | |
| | | | | | | (bZlP) | |
| 186 | GmaAffx.47590.1.S1_at | 0.39 | CA853861 | Metabolism | | | UDP-glucuronosyl and UDP-glucosyl |
| | | | | | | | transferase |
| 187 | Gma.4822.1.S1_at | 0.388 | BI969249 | Disease & Defense | | | |
| 188 | GmaAffx.63822.1.S1_at | 0.388 | BM520838 | Disease & Defense | | | Purple (tartrate-resistant) acid |
| | | | | | | | phosphatase |
| 189 | GmaAffx.87547.2.S1_at | 0.388 | BQ080473 | Cell Structure | | | Beta-glucosidase, lactase |
| | | | | | | | phlorizinhydrolase, and related proteins |
| 190 | GmaAffx.78297.1.S1_at | 0.387 | BM731185 | Unclassified - Hypothetical | | | FOG: Predicted E3 ubiquitin ligase |
| | | | | Protein Supported by cDNA | | | |
| 191 | Gma.10827.2.S1_s_at | 0.386 | BQ612452 | Protein Destination & Storage | Storage | | |
| | | | | | Protein | | |
| 192 | GmaAffx.82000.1.S1_at | 0.385 | BI094895 | Unclassified - Hypothetical | | | |
| | | | | Protein Supported by cDNA | | | |
| 193 | GmaAffx.24451.1.A1_at | 0.384 | BU550405 | Metabolism | Cytochrome | CYP90C | Cytochrome P450 CYP4/CYP19/CYP26 |
| | | | | | P450 | | subfamilies |
| 194 | GmaAffx.84338.1.S1_at | 0.384 | AI856789 | Unclassified - Hypothetical | | | Predicted E3 ubiquitin ligase |
| | | | | Protein Supported by cDNA | | | |
| 195 | GmaAffx.53108.1.S1_at | 0.384 | BM093748 | Signal Transduction | Protein | | Serine/threonine protein kinase |
| | | | | | Kinase | | |
| 196 | Gma.16875.1.A1_at | 0.383 | CD406723 | Metabolism | | | Aldo/keto reductase family proteins |
| 197 | GmaAffx.44484.1.S1_at | 0.383 | BE475213 | Unclassified - Hypothetical | | | |
| | | | | Protein NOT Supported by | | | |
| | | | | cDNA | | | |
| 198 | Gma.17070.1.S1_at | 0.382 | BE822463 | Disease & Defense | | | O-methyltransferase |
| 199 | Gma.2376.1.S1_at | 0.382 | CA852095 | Transcription | Transcription | Homeodomain | Transcription factor MEIS1 and related |
| | | | | | Factor | | HOX domain proteins |
| 200 | Gma.16842.1.A1_at | 0.382 | CD392652 | No Homology to Known | | | |
| | | | | Proteins | | | |
| 201 | Gma.2686.1.S1_s_at | 0.381 | BI970455 | Secondary Metabolism | | | |
| 202 | GmaAffx.90570.1.S1_at | 0.381 | CF806295 | Unclassified - Hypothetical | | | |
| | | | | Protein Supported by cDNA | | | |
| 203 | Gma.7494.1.S1_at | 0.38 | BU548105 | Signal Transduction | Protein | | Serine/threonine protein phosphatase |
| | | | | | Phosphatase | | |
| 204 | GmaAffx.1301.68.S1_s_at | 0.378 | BI974374 | Intracellular Traffic | | | Annex in |
| 205 | GmaAffx.15983.2.S1_s_at | 0.377 | AW279540 | Signal Transduction | Protein | | Serine/threonine protein phosphatase |
| | | | | | Phosphatase | | |
| 206 | GmaAffx.88786.1.A1_s_at | 0.376 | CK606552 | Cell Structure | | | O-methyltransferase |
| 207 | Gma.5478.1.S1_at | 0.374 | AW350606 | Metabolism | | | |
| 208 | Gma.6550.3.S1_s_at | 0.374 | AW234450 | Metabolism | | | Hydroxyindole-O-methyltransferase and |
| | | | | | | | related SAM-dependent |
| | | | | | | | methyltransferases |
| 209 | Gma.1490.1.S1_at | 0.374 | AB000097.1 | Cell Structure | | | Chitinase |
| 210 | GmaAffx.11999.1.S1_at | 0.374 | AW567661 | No Homology to Known | | | |
| | | | | Proteins | | | |
| 211 | GmaAffx.41990.1.S1_at | 0.374 | CA936708 | No Homology to Known | | | |
| | | | | Proteins | | | |
| 212 | GmaAffx.66451.1.S1_at | 0.373 | AW309198 | Secondary Metabolism | | | |
| 213 | GmaAffx.79888.1.S1_at | 0.372 | AI988123 | Signal Transduction | Protein | | |
| | | | | | Kinase | | |
| 214 | Gma.3420.1.S1_at | 0.37 | CD406082 | Unclassified - Hypothetical | | | |
| | | | | Protein Supported by cDNA | | | |
| 215 | GmaAffx.80098.1.S1_at | 0.37 | BG042861 | Metabolism | | | Arylacetamide deacetylase |
| 216 | GmaAffx.1128.1.S1_at | 0.37 | AW348608 | Unclassified - Protein with | | | Copper chaperone |
| | | | | Unknown Function | | | |
| 217 | Gma.12094.1.A1_at | 0.37 | CD395088 | Unclassified - Hypothetical | | | |
| | | | | Protein Supported by cDNA | | | |
| 218 | GmaAffx.90812.1.S1_s_at | 0.37 | CF806537 | Disease & Defense | | | |
| 219 | GmaAffx.9926.2.S1_at | 0.368 | BF597587 | Unclassified - Hypothetical | | | |
| | | | | Protein Supported by cDNA | | | |
| 220 | Gma.6640.1.S1_at | 0.367 | BQ628278 | Disease & Defense | | | |
| 221 | GmaAffx.1301.59.S1_at | 0.367 | BI974591 | Intracellular Traffic | | | Annexin |
| 222 | GmaAffx.21041.1.A1_at | 0.367 | AW350819 | No Homology to Known | | | |
| | | | | Proteins | | | |
| 223 | Gma.16359.1.A1_at | 0.366 | BI970359 | Metabolism | | | GPI-alpha-mannosyltransferase III |
| | | | | | | | (GPI10/PIG-B) involved in |
| | | | | | | | glycosylphosphatidylinositol anchor |
| | | | | | | | biosynthesis |
| 224 | Gma.1195.1.S1_s_at | 0.366 | AI442801 | Metabolism | | | Predicted flavoprotein |
| 225 | GmaAffx.91205.1.S1_at | 0.365 | CF807869 | Metabolism | | | Glyoxylate/hydroxypyruvate reductase |
| | | | | | | | (D-isomer-specific 2-hydroxy acid |
| | | | | | | | dehydrogenase superfamily) |
| 226 | GmaAffx.93342.1.S1_s_at | 0.364 | CF809067 | Disease & Defense | | | Glutathione peroxidase |
| 227 | GmaAffx.74899.1.S1_at | 0.364 | CA852377 | No Homology to Known | | | |
| | | | | Proteins | | | |
| 228 | GmaAffx.15613.1.S1_at | 0.364 | AI939073 | Unclassified - Hypothetical | | | |
| | | | | Protein NOT Supported by | | | |
| | | | | cDNA | | | |
| 229 | GmaAffx.18953.1.S1_at | 0.363 | BM178798 | No Homology to Known | | | |
| | | | | Proteins | | | |
| 230 | GmaAffx.46837.1.S1_at | 0.362 | BU550919 | Secondary Metabolism | | | Multicopper oxidases |
| 231 | GmaAffx.50646.1.A1_at | 0.362 | AW310451 | Unclassified - Hypothetical | | | Molybdenum cofactor sulfurase |
| | | | | Protein Supported by cDNA | | | |
| 232 | Gma.2887.2.S1_x_at | 0.362 | BI701520 | Secondary Metabolism | | | Phenylalanine and histidine ammonia- |
| | | | | | | | lyase |
| 233 | Gma.17835.1.S1_at | 0.361 | BE822840 | Metabolism | | | |
| 234 | Gma.6719.2.S1_at | 0.361 | BG726773 | No Homology to Known | | | |
| | | | | Proteins | | | |
| 235 | GmaAffx.86927.1.S1_at | 0.36 | CA937162 | No Homology to Known | | | |
| | | | | Proteins | | | |
| 236 | Gma.3804.1.A1_at | 0.36 | AW310249 | Disease & Defense | | | |
| 237 | GmaAffx.93189.1.S1_s_at | 0.359 | CF808914 | Metabolism | | | Carbon-nitrogen hydrolase |
| 238 | Gma.821.1.S1_at | 0.357 | AF243371.1 | Metabolism | | | Glutathione S-transferase |
| 239 | GmaAffx.18599.1.S1_at | 0.357 | BI787464 | Secondary Metabolism | Amines | | Amine oxidase |
| | | | | | Biosynthesis | | |
| 240 | Gma.5634.1.S1_at | 0.356 | BE806891 | Secondary Metabolism | | | Steroid reductase |
| 241 | GmaAffx.74256.1.S1_at | 0.356 | BE057293 | No Homology to Known | | | |
| | | | | Proteins | | | |
| 242 | Gma.2086.1.S1_at | 0.355 | AF035671.1 | Metabolism | Amino Acid | | Arginase family protein |
| | | | | | Biosynthesis/ | | |
| | | | | | Metabolism | | |
| 243 | Gma.73.1.S1_at | 0.354 | AF098662.1 | Protein Destination & Storage | | | |
| 244 | GmaAffx.58125.1.A1_at | 0.353 | BE347561 | Transcription | Transcription | Basic Helix- | |
| | | | | | Factor | Loop-Helix | |
| | | | | | | (bHLH) | |
| 245 | Gma.12731.2.A1_s_at | 0.353 | CD395848 | Unclassified - Hypothetical | | | Aquaporin (major intrinsic protein |
| | | | | Protein NOT Supported by | | | family) |
| | | | | cDNA | | | |
| 246 | GmaAffx.87793.1.S1_at | 0.353 | BI941782 | Unclassified - Hypothetical | | | |
| | | | | Protein NOT Supported by | | | |
| | | | | cDNA | | | |
| 247 | GmaAffx.6790.1.S1_at | 0.352 | AI856683 | Disease & Defense | | | Soluble epoxide hydrolase |
| 248 | GmaAffx.15471.1.S1_at | 0.352 | BQ611747 | Transcription | Transcription | MYB | Transcription factor, Myb superfamily |
| | | | | | Factor | | |
| 249 | GmaAffx.92192.1.S1_s_at | 0.352 | CF806406 | Disease & Defense | | | |
| 250 | Gma.1929.1.S1_at | 0.352 | BU083650 | Transcription | Transcription | Homeodomain | Transcription factor HEX, contains |
| | | | | | Factor | | HOX and HALZ domains |
| 251 | Gma.17739.1.S1_at | 0.352 | AW310535 | Unclassified - Hypothetical | | | |
| | | | | Protein Supported by cDNA | | | |
| 252 | GmaAffx.59014.1.S1_s_at | 0.35 | AW733977 | No Homology to Known | | | |
| | | | | Proteins | | | |
| 253 | Gma.7120.2.S1_a_at | 0.35 | BG041285 | Metabolism | | | Alanine-glyoxylate aminotransferase |
| | | | | | | | AGT2 |
| 254 | GmaAffx.478.1.S1_at | 0.348 | BE822248 | Transcription | Transcription | Homeodomain | Transcription factor MEIS1 and related |
| | | | | | Factor | | HOX domain proteins |
| 255 | GmaAffx.11892.1.S1_at | 0.348 | BE820605 | Secondary Metabolism | | | Iron/ascorbate family oxidoreductases |
| 256 | Gma.3888.2.S1_at | 0.347 | BM139532 | Unclassified - Hypothetical | | | Aldehyde reductase |
| | | | | Protein NOT Supported by | | | |
| | | | | cDNA | | | |
| 257 | GmaAffx.82218.1.S1_at | 0.347 | BI702052 | Secondary Metabolism | | | Iron/ascorbate family oxidoreductases |
| 258 | GmaAffx.93642.1.S1_s_at | 0.347 | CF809367 | Disease & Defense | | | |
| 259 | Gma.6047.1.S1_s_at | 0.346 | BQ611159 | Unclassified - Protein with | | | |
| | | | | Unknown Function | | | |
| 260 | Gma.5634.1.S1_s_at | 0.344 | BE806891 | Secondary Metabolism | | | Steroid reductase |
| 261 | Gma.10969.3.S1_x_at | 0.343 | CD396552 | Metabolism | | | |
| 262 | Gma.18006.1.S1_at | 0.343 | CF922923 | Transcription | Transcription | Homeodomain | Transcription factor MEIS1 and related |
| | | | | | Factor | | HOX domain proteins |
| 263 | GmaAffx.62195.1.S1_at | 0.343 | AW472439 | No Homology to Known | | | |
| | | | | Proteins | | | |
| 264 | Gma.10854.1.S1_at | 0.343 | CA802334 | No Homology to Known | | | |
| | | | | Proteins | | | |
| 265 | Gma.5258.1.S1_at | 0.341 | BE658899 | No Homology to Known | | | |
| | | | | Proteins | | | |
| 266 | Gma.4401.2.S1_a_at | 0.339 | BI970725 | Disease & Defense | | | |
| 267 | GmaAffx.75740.1.S1_at | 0.338 | BG157373 | Unclassified - Hypothetical | | | |
| | | | | Protein NOT Supported by | | | |
| | | | | cDNA | | | |
| 268 | GmaAffx.64261.1.A1_at | 0.338 | BI969080 | Unclassified - Protein with | | | 1,4-benzoquinone reductase-like; Trp |
| | | | | Unknown Function | | | repressor binding protein-like/protoplast- |
| | | | | | | | secreted protein |
| 269 | Gma.4981.1.A1_at | 0.337 | BI970256 | No Homology to Known | | | |
| | | | | Proteins | | | |
| 270 | Gma.3522.1.S1_at | 0.336 | BM140209 | Unclassified - Hypothetical | | | |
| | | | | Protein Supported by cDNA | | | |
| 271 | GmaAffx.30792.1.A1_at | 0.335 | BU544815 | Unclassified - Hypothetical | | | |
| | | | | Protein Supported by cDNA | | | |
| 272 | Gma.7437.1.S1_at | 0.332 | BU547705 | Unclassified - Hypothetical | | | |
| | | | | Protein Supported by cDNA | | | |
| 273 | Gma.7254.1.S1_at | 0.332 | BU546166 | Unclassified - Hypothetical | | | |
| | | | | Protein Supported by cDNA | | | |
| 274 | GmaAffx.91312.1.S1_s_at | 0.332 | CF806002 | Transcription | Transcription | Homeodomain | Transcription factor HEX, contains |
| | | | | | Factor | | HOX and HALZ domains |
| 275 | Gma.14075.1.S1_at | 0.331 | CD415333 | Protein Destination & Storage | Storage | | Ca2+-independent phospholipase A2 |
| | | | | | Protein | | |
| 276 | Gma.15155.2.S1_at | 0.33 | BG789619 | Unclassified - Hypothetical | | | |
| | | | | Protein Supported by cDNA | | | |
| 277 | Gma.1622.1.A1_s_at | 0.33 | BU090523 | Signal Transduction | Protein | | FOG: Transposon-encoded proteins with |
| | | | | | Kinase | | TYA, reverse transcriptase, integrase |
| | | | | | | | domains in various combinations |
| 278 | GmaAffx.61872.1.S1_at | 0.329 | AW102386 | Signal Transduction | Protein | | |
| | | | | | Kinase | | |
| 279 | GmaAffx.50673.2.S1_s_at | 0.329 | BI787147 | Transcription | Transcription | NAC | |
| | | | | | Factor | Domain | |
| 280 | GmaAffx.26868.1.S1_at | 0.328 | BF325443 | Unclassified - Hypothetical | | | Tomosyn and related SNARE- |
| | | | | Protein NOT Supported by | | | interacting proteins |
| | | | | cDNA | | | |
| 281 | GmaAffx.91185.1.S1_at | 0.327 | CF806910 | Cell Structure | | | Pectin acetylesterase and similar proteins |
| 282 | GmaAffx.47230.1.S1_at | 0.327 | CA820051 | Transporter | | | Amino acid transporters |
| 283 | Gma.5622.1.S1_at | 0.326 | BG363493 | No Homology to Known | | | |
| | | | | Proteins | | | |
| 284 | Gma.5513.1.S1_x_at | 0.325 | AW310453 | Metabolism | | | Haloacid dehalogenase-like hydrolase |
| 285 | GmaAffx.26859.1.S1_s_at | 0.325 | BM085620 | Disease & Defense | | | Glutathione S-transferase |
| 286 | Gma.8490.1.S1_at | 0.323 | AB030492.1 | Metabolism | | | Protein involved in thiamine |
| | | | | | | | biosynthesis and DNA damage tolerance |
| 287 | Gma.10342.1.S1_at | 0.323 | BE058741 | Unclassified - Hypothetical | | | |
| | | | | Protein Supported by cDNA | | | |
| 288 | Gma.15605.1.S1_at | 0.321 | BM143798 | Transcription | Transcription | AP2/EREBP | |
| | | | | | Factor | | |
| 289 | GmaAffx.19118.1.S1_at | 0.32 | BM523731 | Unclassified - Hypothetical | | | |
| | | | | Protein NOT Supported by | | | |
| | | | | cDNA | | | |
| 290 | GmaAffx.56301.1.S1_at | 0.32 | BQ610805 | No Homology to Known | | | |
| | | | | Proteins | | | |
| 291 | Gma.6046.1.S1_at | 0.32 | BE022758 | Unclassified - Hypothetical | | | |
| | | | | Protein NOT Supported by | | | |
| | | | | cDNA | | | |
| 292 | GmaAffx.92166.1.S1_s_at | 0.318 | CF806313 | Signal Transduction | Unclassified | | Aspartyl protease |
| 293 | GmaAffx.3711.1.A1_at | 0.318 | BE822116 | Unclassified - Hypothetical | | | |
| | | | | Protein NOT Supported by | | | |
| | | | | cDNA | | | |
| 294 | Gma.3745.1.S1_at | 0.318 | AW156339 | Secondary Metabolism | Amines | | Amine oxidase |
| | | | | | Biosynthesis | | |
| 295 | Gma.17798.2.S1_at | 0.317 | CD412748 | No Homology to Known | | | |
| | | | | Proteins | | | |
| 296 | GmaAffx.21924.1.S1_at | 0.316 | BQ611867 | Disease & Defense | | | |
| 297 | GmaAffx.37615.1.S1_at | 0.315 | BQ454150 | No Homology to Known | | | |
| | | | | Proteins | | | |
| 298 | Gma.1394.1.S1_at | 0.315 | BG839300 | Metabolism | | | |
| 299 | GmaAffx.89772.10.A1_s_at | 0.315 | CK606097 | Disease & Defense | | | |
| 300 | Gma.5215.1.S1_at | 0.313 | AI988141 | Unclassified - Hypothetical | | | BCL2-associated athanogene-like |
| | | | | Protein Supported by cDNA | | | proteins and related BAG family |
| | | | | | | | chaperone regulators |
| 301 | Gma.6623.1.S1_at | 0.313 | BI702268 | Unclassified - Protein with | | | 1,4-benzoquinone reductase-like; Trp |
| | | | | Unknown Function | | | repressor binding protein-like/protoplast- |
| | | | | | | | secreted protein |
| 302 | GmaAffx.21978.1.A1_at | 0.311 | CA803009 | No Homology to Known | | | |
| | | | | Proteins | | | |
| 303 | GmaAffx.76346.1.A1_at | 0.311 | BQ612378 | Energy | | | 1,4-benzoquinone reductase-like; Trp |
| | | | | | | | repressor binding protein-like/protoplast- |
| | | | | | | | secreted protein |
| 304 | Gma.1137.1.S1_x_at | 0.31 | BF067710 | Metabolism | | | |
| 305 | GmaAffx.81362.1.S1_at | 0.309 | BF324988 | Cell Structure | | | |
| 306 | GmaAffx.85528.1.S1_at | 0.309 | BE608070 | Unclassified - Hypothetical | | | FOG: PPR repeat |
| | | | | Protein Supported by cDNA | | | |
| 307 | GmaAffx.55124.1.S1_at | 0.309 | BI970821 | Unclassified - Hypothetical | | | Metacaspase involved in regulation of |
| | | | | Protein Supported by cDNA | | | apoptosis |
| 308 | GmaAffx.77269.1.S1_at | 0.308 | BM093698 | No Homology to Known | | | |
| | | | | Proteins | | | |
| 309 | Gma.15568.1.S1_at | 0.305 | AI442650 | Disease & Defense | | | |
| 310 | Gma.17686.1.S1_at | 0.304 | BF597378 | Metabolism | Cytochrome | CYP76C | Cytochrome P450 CYP2 subfamily |
| | | | | | P450 | | |
| 311 | Gma.15469.1.S1_at | 0.302 | BU546655 | Cell Structure | | | Alpha tubulin |
| 312 | Gma.15155.2.S1_a_at | 0.302 | BG789619 | Unclassified - Hypothetical | | | |
| | | | | Protein Supported by cDNA | | | |
| 313 | GmaAffx.57748.1.S1_at | 0.302 | AW306274 | No Homology to Known | | | |
| | | | | Proteins | | | |
| 314 | Gma.3976.1.S1_at | 0.301 | BI972268 | No Homology to Known | | | |
| | | | | Proteins | | | |
| 315 | GmaAffx.61130.1.S1_at | 0.3 | CA799668 | Transporter | | | H+/oligopeptide symporter |
| 316 | GmaAffx.41078.1.S1_at | 0.3 | BI317149 | Metabolism | | | Alcohol dehydrogenase, class 1I1 |
| 317 | Gma.4093.2.S1_at | 0.3 | BQ081460 | Transporter | | | |
| 318 | GmaAffx.62509.2.S1_at | 0.299 | BE021475 | Transcription | Transcription | MYB | Transcription factor, Myb superfamily |
| | | | | | Factor | | |
| 319 | GmaAffx.37055.1.A1_at | 0.299 | BM142939 | Metabolism | | | UDP-glucuronosyl and UDP-glucosyl |
| | | | | | | | transferase |
| 320 | GmaAffx.68724.1.A1_at | 0.299 | BI970438 | No Homology to Known | | | |
| | | | | Proteins | | | |
| 321 | Gma.5867.1.A1_s_at | 0.297 | BI316163 | Metabolism | | | |
| 322 | Gma.752.1.A1_at | 0.297 | AW432463 | Transcription | Transcription | Basic Helix- | |
| | | | | | Factor | Loop-Helix | |
| | | | | | | (bHLH) | |
| 323 | GmaAffx.9926.1.A1_at | 0.296 | CD411291 | No Homology to Known | | | |
| | | | | Proteins | | | |
| 324 | Gma.4249.1.S1_at | 0.296 | BF324893 | Secondary Metabolism | | | Methyltransferase |
| 325 | GmaAffx.78163.1.S1_s_at | 0.296 | BM520857 | Signal Transduction | Protein | | Serine/threonine protein phosphatase |
| | | | | | Phosphatase | | |
| 326 | Gma.968.1.S1_at | 0.296 | D28876.1 | Protein Destination & Storage | | | Asparaginyl peptidases |
| 327 | GmaAffx.38555.2.S1_at | 0.296 | BI425873 | No Homology to Known | | | |
| | | | | Proteins | | | |
| 328 | Gma.7454.1.S1_a_at | 0.296 | CD405901 | Transcription | Transcription | AUX/IAA | |
| | | | | | Factor | | |
| 329 | GmaAffx.93328.1.S1_s_at | 0.295 | CF809053 | Secondary Metabolism | | | |
| 330 | Gma.2475.1.S1_at | 0.295 | BM522941 | Cell Structure | | | |
| 331 | Gma.7120.1.S1_at | 0.293 | BM093481 | Metabolism | | | Alanine-glyoxylate aminotransferase |
| | | | | | | | AGT2 |
| 332 | Gma.1716.1.S1_at | 0.292 | AW156801 | Unclassified - Hypothetical | | | |
| | | | | Protein Supported by cDNA | | | |
| 333 | Gma.14338.1.A1_at | 0.29 | CF923111 | Protein Destination & Storage | Storage | | |
| | | | | | Protein | | |
| 334 | GmaAffx.4968.1.S1_at | 0.289 | BI967691 | Unclassified - Hypothetical | | | |
| | | | | Protein Supported by cDNA | | | |
| 335 | GmaAffx.24102.1.S1_at | 0.286 | BQ453911 | Unclassified - Hypothetical | | | |
| | | | | Protein Supported by cDNA | | | |
| 336 | GmaAffx.70005.1.S1_s_at | 0.284 | CA936335 | Secondary Metabolism | | | Iron/ascorbate family oxidoreductases |
| 337 | Gma.17921.2.S1_s_at | 0.284 | BM085020 | Metabolism | Sucrose | | Glycosyltransferase |
| | | | | | Biosynthesis | | |
| 338 | Gma.5863.2.S1_s_at | 0.283 | AW350762 | Secondary Metabolism | | | |
| 339 | GmaAffx.56715.1.S1_at | 0.283 | BG041616 | Disease & Defense | | | |
| 340 | Gma.364.1.A1_at | 0.283 | AW309875 | Unclassified - Hypothetical | | | |
| | | | | Protein Supported by cDNA | | | |
| 341 | GmaAffx.90970.1.S1_s_at | 0.28 | CF806695 | Unclassified - Hypothetical | | | |
| | | | | Protein Supported by cDNA | | | |
| 342 | GmaAffx.13912.1.S1_at | 0.28 | BI699258 | Disease & Defense | | | Purple (tartrate-resistant) acid |
| | | | | | | | phosphatase |
| 343 | Gma.5513.1.S1_at | 0.277 | AW310453 | Metabolism | | | Haloacid dehalogenase-like hydrolase |
| 344 | GmaAffx.71308.2.A1_at | 0.276 | AW569888 | No Homology to Known | | | |
| | | | | Proteins | | | |
| 345 | Gma.12684.1.S1_at | 0.275 | CD394773 | Transcription | Transcription | Zinc Finger | |
| | | | | | Factor | | |
| 346 | Gma.13150.1.A1_at | 0.274 | CD411577 | Unclassified - Protein with | Hormone | | |
| | | | | Unknown Function | Induced | | |
| 347 | GmaAffx.57469.1.S1_at | 0.274 | AW351054 | Unclassified - Hypothetical | | | |
| | | | | Protein Supported by cDNA | | | |
| 348 | Gma.2842.1.S1_at | 0.274 | BG511652 | Unclassified - Protein with | | | Predicted Yippee-type zinc-binding |
| | | | | Unknown Function | | | protein |
| 349 | GmaAffx.86745.1.S1_s_at | 0.271 | BI424960 | Disease & Defense | | | |
| 350 | Gma.6599.1.S1_at | 0.271 | BQ629084 | Cell Structure | | | |
| 351 | GmaAffx.75697.1.S1_s_at | 0.27 | BU090518 | Unclassified - Hypothetical | | | |
| | | | | Protein Supported by cDNA | | | |
| 352 | Gma.6033.1.S1_at | 0.27 | BI967767 | Unclassified - Hypothetical | | | |
| | | | | Protein Supported by cDNA | | | |
| 353 | Gma.4837.1.S1_at | 0.269 | BI969343 | Transporter | | | H+/oligopeptide symporter |
| 354 | Gma.382.1.A1_at | 0.268 | AW349068 | Metabolism | | | Arylacetamide deacetylase |
| 355 | GmaAffx.91555.1.A1_s_at | 0.268 | CF807280 | Metabolism | | | |
| 356 | GmaAffx.9911.1.S1_at | 0.267 | BU547808 | Unclassified - Hypothetical | | | Tomosyn and related SNARE- |
| | | | | Protein NOT Supported by | | | interacting proteins |
| | | | | cDNA | | | |
| 357 | Gma.3604.1.S1_at | 0.267 | AW349604 | Cell Structure | | | O-methyltransferase |
| 358 | Gma.17840.1.S1_at | 0.267 | BE821995 | No Homology to Known | | | |
| | | | | Proteins | | | |
| 359 | Gma.9086.1.S1_at | 0.263 | AW184932 | Cell Growth & Division | | | |
| 360 | GmaAffx.71842.1.S1_at | 0.263 | BE555317 | Metabolism | | | Possible pfkB family carbohydrate |
| | | | | | | | kinase |
| 361 | GmaAffx.86247.1.S1_s_at | 0.262 | BG238674 | Unclassified - Hypothetical | | | |
| | | | | Protein Supported by cDNA | | | |
| 362 | GmaAffx.62509.1.S1_s_at | 0.261 | BE022332 | Transcription | Transcription | MYB | Transcription factor, Myb superfamily |
| | | | | | Factor | | |
| 363 | GmaAffx.90104.1.S1_s_at | 0.26 | CF805829 | Transporter | | | Permease of the major facilitator |
| | | | | | | | superfamily |
| 364 | Gma.4874.1.A1_at | 0.259 | AI938293 | No Homology to Known | | | |
| | | | | Proteins | | | |
| 365 | Gma.3914.1.S1_at | 0.258 | BI702206 | Unclassified - Hypothetical | | | |
| | | | | Protein Supported by cDNA | | | |
| 366 | GmaAffx.84751.1.A1_at | 0.257 | AW349702 | Transcription | Transcription | MYB | Transcription factor, Myb superfamily |
| | | | | | Factor | | |
| 367 | GmaAffx.11172.1.A1_at | 0.256 | CD404556 | No Homology to Known | | | |
| | | | | Proteins | | | |
| 368 | GmaAffx.93591.1.S1_s_at | 0.256 | CF809316 | Metabolism | | | |
| 369 | Gma.6343.1.S1_at | 0.25 | AI442963 | Unclassified - Hypothetical | | | |
| | | | | Protein NOT Supported by | | | |
| | | | | cDNA | | | |
| 370 | Gma.6999.2.S1_s_at | 0.249 | X60043.1 | Disease & Defense | | | |
| 371 | Gma.4222.1.S1_at | 0.248 | BU083971 | Unclassified - Hypothetical | | | Arylacetamide deacetylase |
| | | | | Protein NOT Supported by | | | |
| | | | | cDNA | | | |
| 372 | Gma.3987.1.S1_at | 0.247 | BQ453408 | Unclassified - Hypothetical | | | Sodium sulfate symporter and related |
| | | | | Protein NOT Supported by | | | arsenite permeases |
| | | | | cDNA | | | |
| 373 | Gma.9828.1.S1_at | 0.245 | AF529301.1 | Metabolism | | | Haloacid dehalogenase-like hydrolase |
| 374 | GmaAffx.89259.1.S1_s_at | 0.245 | CK605646 | Metabolism | | | |
| 375 | GmaAffx.70008.1.S1_at | 0.244 | BU762337 | Transcription | Transcription | MYB | Transcription factor, Myb superfamily |
| | | | | | Factor | | |
| 376 | Gma.7422.1.S1_at | 0.24 | BQ612005 | Transporter | | | |
| 377 | Gma.11119.2.S1_s_at | 0.24 | CF920743 | Disease & Defense | | | |
| 378 | Gma.3744.1.S1_at | 0.239 | CD417438 | Unclassified - Protein With | | | |
| | | | | Unknown Function | | | |
| 379 | Gma.1137.1.S1_a_at | 0.239 | BF067710 | Metabolism | | | |
| 380 | GmaAffx.50980.1.S1_at | 0.238 | BE823095 | Signal Transduction | Protein | | |
| | | | | | Phosphatase | | |
| 381 | GmaAffx.41917.1.S1_at | 0.238 | BE658926 | Disease & Defense | | | |
| 382 | Gma.11166.1.S1_s_at | 0.237 | AW705829 | Metabolism | | | |
| 383 | GmaAffx.71308.1.S1_at | 0.236 | BF595112 | Transcription | Transcription | Heat Shock | Heat shock transcription factor |
| | | | | | Factor | | |
| 384 | Gma.398.2.S1_at | 0.236 | BU925900 | Metabolism | | | Homocysteine S-methyltransferase |
| 385 | Gma.3161.2.S1_at | 0.235 | CD400025 | Secondary Metabolism | | | Iron/ascorbate family oxidoreductases |
| 386 | GmaAffx.83818.1.S1_at | 0.235 | BU762716 | No Homology to Known | | | |
| | | | | Proteins | | | |
| 387 | GmaAffx.8999.2.A1_s_at | 0.234 | BI700574 | No Homology to Known | | | |
| | | | | Proteins | | | |
| 388 | Gma.4796.1.A1_at | 0.234 | AW102323 | Unclassified - Hypothetical | | | Uncharacterized conserved protein |
| | | | | Protein NOT Supported by | | | |
| | | | | cDNA | | | |
| 389 | Gma.15093.1.S1_at | 0.234 | CD400374 | Protein Destination & Storage | | | Glutathione S-transferase |
| 390 | GmaAffx.15119.1.A1_at | 0.234 | BU544528 | Metabolism | | | Possible pfkB family carbohydrate |
| | | | | | | | kinase |
| 391 | GmaAffx.87433.1.S1_at | 0.234 | BI701509 | Unclassified - Hypothetical | | | |
| | | | | Protein Supported by cDNA | | | |
| 392 | Gma.11166.1.S1_x_at | 0.233 | AW705829 | Metabolism | | | |
| 393 | GmaAffx.74323.1.S1_at | 0.233 | BI426587 | Unclassified - Hypothetical | | | Endoplasmic reticulum protein EP58, |
| | | | | Protein NOT Supported by | | | contains filamin rod domain and KDEL |
| | | | | cDNA | | | motif |
| 394 | GmaAffx.50980.1.S1_x_at | 0.232 | BE823095 | Signal Transduction | Protein | | |
| | | | | | Phosphatase | | |
| 395 | Gma.3000.1.S1_at | 0.232 | BE657172 | No Homology to Known | | | |
| | | | | Proteins | | | |
| 396 | GmaAffx.47739.1.S1_at | 0.228 | BM522736 | Unclassified - Protein with | | | |
| | | | | Unknown Function | | | |
| 397 | Gma.1527.1.S1_x_at | 0.227 | BI968185 | Secondary Metabolism | | | Flavonol reductase/cinnamoyl-CoA |
| | | | | | | | reductase |
| 398 | Gma.18006.1.A1_at | 0.227 | AI496486 | Transcription | Transcription | Homeodomain | Transcription factor ME1S1 and related |
| | | | | | Factor | | HOX domain proteins |
| 399 | Gma.6999.1.S1_x_at | 0.225 | AF529303.1 | Disease & Defense | | | |
| 400 | GmaAffx.45003.1.S1_at | 0.224 | BI968048 | Unclassified - Hypothetical | | | Uncharacterized conserved protein |
| | | | | Protein Supported by cDNA | | | |
| 401 | GmaAffx.91338.1.S1_at | 0.222 | CF807063 | Unclassified - Hypothetical | | | Predicted haloacid dehalogenase-like |
| | | | | Protein Supported by cDNA | | | hydrolase |
| 402 | Gma.5331.1.S1_at | 0.221 | BI892702 | Transcription | Transcription | NAC | |
| | | | | | Factor | Domain | |
| 403 | GmaAffx.2026.1.A1_at | 0.216 | BU550139 | Transcription | Transcription | NAC | |
| | | | | | Factor | Domain | |
| 404 | Gma.3175.1.S1_at | 0.216 | AI736154 | Unclassified - Hypothetical | | | |
| | | | | Protein NOT Supported by | | | |
| | | | | cDNA | | | |
| 405 | Gma.3744.2.S1_a_at | 0.215 | BI892693 | Unclassified - Protein With | | | |
| | | | | Unknown Function | | | |
| 406 | Gma.2252.1.S1_at | 0.215 | CD406598 | Unclassified - Hypothetical | | | |
| | | | | Protein Supported by cDNA | | | |
| 407 | Gma.10227.1.A1_at | 0.215 | BE020065 | Metabolism | | | Flavin-containing monooxygenase |
| 408 | GmaAffx.12933.1.S1_at | 0.211 | AW395787 | Transcription | Transcription | CCAAT | CCAAT-binding factor, subunit B |
| | | | | | Factor | | (HAP2) |
| 409 | GmaAffx.23292.1.A1_at | 0.211 | BI967795 | No Homology to Known | | | |
| | | | | Proteins | | | |
| 410 | Gma.7549.1.S1_at | 0.21 | BG508637 | Unclassified - Protein with | | | |
| | | | | Unknown Function | | | |
| 411 | Gma.13218.1.A1_s_at | 0.209 | CD391892 | No Homology to Known | | | |
| | | | | Proteins | | | |
| 412 | GmaAffx.89772.14.A1_s_at | 0.209 | CK606614 | Disease & Defense | | | |
| 413 | Gma.5611.1.S1_at | 0.208 | AW309826 | Metabolism | Cytochrome | CYP81D | Cytochrome P450 CYP2 subfamily |
| | | | | | P450 | | |
| 414 | GmaAffx.5264.1.S1_s_at | 0.207 | BM521512 | Disease & Defense | | | |
| 415 | GmaAffx.24521.1.S1_at | 0.207 | BU761767 | Unclassified - Hypothetical | | | |
| | | | | Protein NOT Supported by | | | |
| | | | | cDNA | | | |
| 416 | GmaAffx.10582.1.S1_at | 0.205 | BM523687 | Transcription | Transcription | CCAAT | CCAAT-binding factor, subunit A |
| | | | | | Factor | | (HAP3) |
| 417 | Gma.6033.2.S1_at | 0.204 | AI416728 | Unclassified - Hypothetical | | | |
| | | | | Protein Supported by cDNA | | | |
| 418 | GmaAffx.14030.1.S1_at | 0.204 | BE021503 | No Homology to Known | | | |
| | | | | Proteins | | | |
| 419 | Gma.18052.1.S1_at | 0.204 | BF595464 | Transporter | | | |
| 420 | GmaAffx.59470.1.S1_at | 0.202 | BI469872 | Secondary Metabolism | | | Phenylalanine and histidine ammonia- |
| | | | | | | | lyase |
| 421 | Gma.3165.1.S1_at | 0.202 | CD406755 | Transcription | Transcription | Homeodomain | Transcription factor HEX, contains |
| | | | | | Factor | | HOX and HALZ domains |
| 422 | Gma.1326.1.S1_at | 0.198 | CD397515 | Secondary Metabolism | | | |
| 423 | Gma.6192.1.S1_at | 0.197 | AW396773 | Metabolism | | | |
| 424 | GmaAffx.61593.1.S1_at | 0.197 | BU765478 | Unclassified - Hypothetical | | | Sodium sulfate symporter and related |
| | | | | Protein NOT Supported by | | | arsenite permeases |
| | | | | cDNA | | | |
| 425 | Gma.9638.1.A1_at | 0.195 | CA936403 | Disease & Defense | | | |
| 426 | GmaAffx.71222.1.S1_at | 0.194 | AW460050 | Metabolism | | | 5′-AMP-activated protein kinase, gamma |
| | | | | | | | subunit |
| 427 | GmaAffx.81618.1.S1_at | 0.194 | AW706403 | No Homology to Known | | | |
| | | | | Proteins | | | |
| 428 | Gma.3591.2.S1_at | 0.193 | BE473658 | Secondary Metabolism | Ethylene | | Iron/ascorbate family oxidoreductases |
| | | | | | Biosynthesis | | |
| 429 | Gma.1269.1.S1_at | 0.192 | AW309671 | Metabolism | | | Flavonol reductase/cinnamoyl-CoA |
| | | | | | | | reductase |
| 430 | Gma.6643.1.A1_at | 0.191 | BE823205 | Secondary Metabolism | | | Sulfotransferase |
| 431 | Gma.8370.1.S1_at | 0.191 | BQ454087 | Signal Transduction | Protein | | Serine/threonine protein phosphatase |
| | | | | | Phosphatase | | |
| 432 | GmaAffx.57967.1.S1_at | 0.19 | AW102170 | Protein Destination & Storage | | | |
| 433 | Gma.5469.1.S1_at | 0.19 | AW349200 | No Homology to Known | | | |
| | | | | Proteins | | | |
| 434 | GmaAffx.70435.2.S1_at | 0.189 | AW705853 | Transcription | Transcription | Basic Helix- | |
| | | | | | Factor | Loop-Helix | |
| | | | | | | (bHLH) | |
| 435 | Gma.6081.1.S1_at | 0.187 | BI967474 | Disease & Defense | | | Predicted chitinase |
| 436 | Gma.1394.2.S1_at | 0.186 | BI470370 | Metabolism | | | |
| 437 | Gma.17877.1.S1_at | 0.185 | BQ612471 | Unclassified - Protein with | | | |
| | | | | Unknown Function | | | |
| 438 | Gma.15996.1.S1_x_at | 0.184 | BQ454193 | Unclassified - Hypothetical | | | |
| | | | | Protein Supported by cDNA | | | |
| 439 | Gma.6075.1.S1_at | 0.184 | AW317183 | Disease & Defense | | | |
| 440 | Gma.16947.1.S1_at | 0.184 | BG155375 | Unclassified - Hypothetical | | | |
| | | | | Protein Supported by cDNA | | | |
| 441 | Gma.7788.1.A1_at | 0.182 | BU549148 | Unclassified - Hypothetical | | | Aspartyl protease |
| | | | | Protein Supported by cDNA | | | |
| 442 | Gma.8489.1.S1_s_at | 0.181 | AB030493.1 | Metabolism | | | Protein involved in thiamine |
| | | | | | | | biosynthesis and DNA damage tolerance |
| 443 | Gma.1527.1.S1_a_at | 0.18 | BI968185 | Secondary Metabolism | | | Flavonol reductase/cinnamoyl-CoA |
| | | | | | | | reductase |
| 444 | GmaAffx.90395.1.A1_s_at | 0.177 | CF807499 | Unclassified - Protein with | | | |
| | | | | Unknown Function | | | |
| 445 | Gma.6643.2.S1_at | 0.177 | BE023121 | Secondary Metabolism | | | Sulfotransferase |
| 446 | Gma.6999.1.S1_s_at | 0.174 | AF529303.1 | Disease & Defense | | | |
| 447 | GmaAffx.5531.1.S1_at | 0.167 | BQ740711 | Cell Structure | | | |
| 448 | Gma.15534.1.S1_at | 0.162 | U50081.1 | Metabolism | | | |
| 449 | Gma.3578.1.S1_at | 0.162 | AW156522 | Disease & Defense | | | Acyl-CoA synthetase |
| 450 | Gma.6999.3.S1_s_at | 0.162 | CF921432 | Disease & Defense | | | |
| 451 | GmaAffx.59808.1.S1_at | 0.161 | BM886228 | Transporter | | | Predicted transporter (major facilitator |
| | | | | | | | superfamily) |
| 452 | GmaAffx.11897.1.S1_at | 0.16 | BE819868 | Transporter | | | Permease of the major facilitator |
| | | | | | | | superfamily |
| 453 | GmaAffx.295.1.A1_s_at | 0.159 | BG653267 | Protein Destination & Storage | | | |
| 454 | Gma.5309.1.S1_at | 0.158 | BM092263 | Unclassified - Hypothetical | | | |
| | | | | Protein NOT Supported by | | | |
| | | | | cDNA | | | |
| 455 | GmaAffx.70959.1.S1_at | 0.157 | BE611219 | Unclassified - Hypothetical | | | Glucose-6-phosphate/phosphate and |
| | | | | Protein NOT Supported by | | | phosphoenolpyruvate/phosphate |
| | | | | cDNA | | | antiporter |
| 456 | GmaAffx.91428.1.A1_at | 0.157 | CF807137 | Metabolism | | | |
| 457 | GmaAffx.19467.1.S1_at | 0.155 | BQ453237 | Signal Transduction | Protein | | Serine/threonine protein kinase |
| | | | | | Kinase | | |
| 458 | GmaAffx.40923.1.S1_at | 0.154 | BG653848 | Disease & Defense | | | Selenium-binding protein |
| 459 | Gma.8187.1.S1_a_at | 0.154 | BU549133 | Signal Transduction | Protein | | Serine/threonine protein phosphatase |
| | | | | | Phosphatase | | |
| 460 | Gma.9010.1.S1_at | 0.153 | CF921194 | Disease & Defense | | | |
| 461 | GmaAffx.32606.1.S1_at | 0.153 | BM270707 | Unclassified - Hypothetical | | | Aquaporin (major intrinsic protein |
| | | | | Protein NOT Supported by | | | family) |
| | | | | cDNA | | | |
| 462 | Gma.11731.1.A1_at | 0.15 | CD392320 | Unclassified - Protein with | | | |
| | | | | Unknown Function | | | |
| 463 | Gma.1758.1.S1_at | 0.15 | CD402046 | Unclassified - Protein with | | | |
| | | | | Unknown Function | | | |
| 464 | Gma.11028.2.S1_s_at | 0.149 | CA782711 | Metabolism | | | Glyoxylate/hydroxypyruvate reductase |
| | | | | | | | (D-isomer-specific 2-hydroxy acid |
| | | | | | | | dehydrogenase superfamily) |
| 465 | Gma.4873.1.S1_at | 0.148 | BI969532 | Signal Transduction | Protein | | Serine/threonine protein kinase |
| | | | | | Kinase | | |
| 466 | Gma.15036.2.A1_at | 0.148 | CD411972 | Unclassified - Protein with | | | Predicted membrane protein |
| | | | | Unknown Function | | | |
| 467 | Gma.17662.2.S1_s_at | 0.146 | BQ630190 | Unclassified - Hypothetical | | | |
| | | | | Protein NOT Supported by | | | |
| | | | | cDNA | | | |
| 468 | GmaAffx.9944.1.S1_at | 0.146 | BM567882 | Cell Structure | | | |
| 469 | Gma.10216.1.S1_at | 0.145 | BI969369 | Metabolism | | | Hydroxyindole-O-methyltransferase and |
| | | | | | | | related SAM-dependent |
| | | | | | | | methyltransferases |
| 470 | Gma.3578.1.S1_s_at | 0.142 | AW156522 | Disease & Defense | | | Acyl-CoA synthetase |
| 471 | GmaAffx.51464.1.S1_at | 0.142 | BQ628397 | No Homology to Known | | | |
| | | | | Proteins | | | |
| 472 | Gma.336.1.A1_at | 0.142 | AW310815 | Metabolism | Sulfur | | |
| | | | | | Metabolism | | |
| 473 | Gma.7580.1.S1_at | 0.139 | BE057763 | Unclassified - Hypothetical | | | |
| | | | | Protein Supported by cDNA | | | |
| 474 | Gma.1445.1.S1_at | 0.138 | BQ454241 | Metabolism | | | Flavonol reductase/cinnamoyl-CoA |
| | | | | | | | reductase |
| 475 | Gma.12227.1.S1_at | 0.136 | BG511671 | Unclassified - Hypothetical | | | |
| | | | | Protein NOT Supported by | | | |
| | | | | cDNA | | | |
| 476 | GmaAffx.68507.1.S1_at | 0.136 | BI699405 | Transcription | Transcription | Heat Shock | Heat shock transcription factor |
| | | | | | Factor | | |
| 477 | GmaAffx.41230.1.A1_at | 0.135 | BI471609 | Unclassified - Hypothetical | | | |
| | | | | Protein NOT Supported by | | | |
| | | | | cDNA | | | |
| 478 | Gma.4260.1.S1_at | 0.134 | BM177256 | Unclassified - Hypothetical | | | |
| | | | | Protein NOT Supported by | | | |
| | | | | cDNA | | | |
| 479 | Gma.320.1.S1_at | 0.13 | AF019116.1 | Disease & Defense | | | |
| 480 | Gma.6895.1.S1_a_at | 0.122 | BU545788 | Metabolism | | | Alanine-glyoxylate aminotransferase |
| | | | | | | | AGT2 |
| 481 | GmaAffx.12665.1.A1_at | 0.121 | BE822073 | Metabolism | | | |
| 482 | Gma.13062.1.S1_at | 0.12 | CD394315 | Metabolism | | | |
| 483 | Gma.6462.2.S1_a_at | 0.119 | BE555263 | Unclassified - Hypothetical | | | |
| | | | | Protein NOT Supported by | | | |
| | | | | cDNA | | | |
| 484 | GmaAffx.65393.1.S1_s_at | 0.119 | BU760770 | No Homology to Known | | | |
| | | | | Proteins | | | |
| 485 | Gma.3774.2.S1_at | 0.118 | CD393365 | Energy | | | Thioredoxin, nucleoredoxin and related |
| | | | | | | | proteins |
| 486 | Gma.2253.1.S1_at | 0.114 | AF169018.1 | Metabolism | | | Reductases with broad range of substrate |
| | | | | | | | specificities |
| 487 | GmaAffx.92479.1.S1_s_at | 0.112 | CF808204 | No Homology to Known | | | |
| | | | | Proteins | | | |
| 488 | Gma.17446.1.A1_at | 0.106 | AI461103 | Unclassified - Protein with | | | |
| | | | | Unknown Function | | | |
| 489 | Gma.13854.1.S1_at | 0.106 | CD408166 | Metabolism | | | |
| 490 | Gma.15.1.S1_at | 0.106 | U66316.1 | Unclassified - Protein with | | | |
| | | | | Unknown Function | | | |
| 491 | GmaAffx.87417.1.S1_at | 0.104 | BM568072 | Metabolism | | | |
| 492 | GmaAffx.52963.1.S1_at | 0.104 | AW310673 | Unclassified - Protein with | | | |
| | | | | Unknown Function | | | |
| 493 | GmaAffx.26908.1.S1_at | 0.101 | BF425834 | Secondary Metabolism | | | Beta, beta-carotene 15,15′-dioxygenase |
| | | | | | | | and related enzymes |
| 494 | Gma.17890.1.S1_at | 0.101 | AW432965 | No Homology to Known | | | |
| | | | | Proteins | | | |
| 495 | GmaAffx.81750.1.S1_at | 0.0985 | AW306336 | No Homology to Known | | | |
| | | | | Proteins | | | |
| 496 | Gma.12393.3.S1_at | 0.098 | BM085488 | Secondary Metabolism | | | Beta, beta-carotene 15,15′-dioxygenase |
| | | | | | | | and related enzymes |
| 497 | GmaAffx.89407.1.A1_s_at | 0.0978 | CK606273 | Transporter | | | |
| 498 | GmaAffx.17961.1.A1_at | 0.0974 | BE657601 | Protein Destination & Storage | | | Molecular chaperones HSP70/HSC70, |
| | | | | | | | HSP70 superfamily |
| 499 | Gma.3519.1.S1_at | 0.0892 | CD403581 | Unclassified - Hypothetical | | | |
| | | | | Protein Supported by cDNA | | | |
| 500 | Gma.9740.1.S1_at | 0.0859 | BQ081058 | Unclassified - Hypothetical | | | |
| | | | | Protein Supported by cDNA | | | |
| 501 | Gma.16566.1.S1_at | 0.0856 | CD400974 | Metabolism | | | Fumarase |
| 502 | Gma.2133.1.S1_at | 0.0855 | AW310071 | Unclassified - Protein with | | | |
| | | | | Unknown Function | | | |
| 503 | Gma.1243.1.S1_at | 0.0845 | AW311027 | Unclassified - Hypothetical | | | |
| | | | | Protein Supported by cDNA | | | |
| 504 | Gma.15625.1.S1_x_at | 0.0841 | U77678.1 | Metabolism | Amino Acid | | Asparagine synthase (glutamine- |
| | | | | | Biosynthesis/ | | hydrolyzing) |
| | | | | | Metabolism | | |
| 505 | Gma.3000.2.S1_a_at | 0.0841 | BM270844 | No Homology to Known | | | |
| | | | | Proteins | | | |
| 506 | GmaAffx.90395.1.S1_s_at | 0.0837 | CF806120 | Unclassified - Protein with | | | |
| | | | | Unknown Function | | | |
| 507 | Gma.2986.1.S1_at | 0.0827 | BM271324 | Metabolism | | | 5′-AMP-activated protein kinase, gamma |
| | | | | | | | subunit |
| 508 | Gma.6192.1.S1_s_at | 0.0814 | AW396773 | Metabolism | | | |
| 509 | GmaAffx.90600.1.S1_at | 0.0806 | CF806325 | Metabolism | | | |
| 510 | Gma.15625.1.S1_a_at | 0.0771 | U77678.1 | Metabolism | Amino Acid | | Asparagine synthase (glutamine- |
| | | | | | Biosynthesis/ | | hydrolyzing) |
| | | | | | Metabolism | | |
| 511 | Gma.4687.1.S1_at | 0.0751 | AW309716 | Secondary Metabolism | | | |
| 512 | Gma.11211.1.S1_s_at | 0.074 | AI442578 | No Homology to Known | | | |
| | | | | Proteins | | | |
| 513 | GmaAffx.24413.1.A1_at | 0.0704 | BU548368 | Metabolism | | | 5′-AMP-activated protein kinase, gamma |
| | | | | | | | subunit |
| 514 | Gma.8365.1.S1_at | 0.07 | CD396813 | Unclassified - Hypothetical | | | |
| | | | | Protein NOT Supported by | | | |
| | | | | cDNA | | | |
| 515 | Gma.2912.1.S1_s_at | 0.0691 | AF117884.1 | Protein Destination & Storage | Storage | | Uncharacterized conserved protein |
| | | | | | Protein | | |
| 516 | Gma.17985.1.S1_at | 0.0678 | CD416450 | Metabolism | | | |
| 517 | GmaAffx.73698.1.S1_at | 0.0673 | BU964780 | Metabolism | | | 5′-AMP-activated protein kinase, gamma |
| | | | | | | | subunit |
| 518 | GmaAffx.90600.1.S1_s_at | 0.065 | CF806325 | Metabolism | | | |
| 519 | GmaAffx.6635.1.S1_at | 0.0639 | CD412718 | Disease & Defense | | | |
| 520 | Gma.17516.1.S1_at | 0.0631 | CD396417 | Transporter | | | Ca2+/H+ antiporter VCX1 and related |
| | | | | | | | proteins |
| 521 | Gma.1527.2.S1_at | 0.0624 | BE658017 | Secondary Metabolism | | | Flavonol reductase/cinnamoyl-CoA |
| | | | | | | | reductase |
| 522 | Gma.176.1.S1_s_at | 0.0618 | AF004810.1 | Protein Destination & Storage | Storage | | |
| | | | | | Protein | | |
| 523 | Gma.1332.1.S1_x_at | 0.0609 | BI970666 | Secondary Metabolism | Ethylene | | 1-aminocyclopropane-1-carboxylate |
| | | | | | Biosynthesis | | synthase, and related proteins |
| 524 | Gma.9947.1.S1_at | 0.0589 | BF324269 | Unclassified - Hypothetical | | | |
| | | | | Protein Supported by cDNA | | | |
| 525 | Gma.8425.1.S1_s_at | 0.0562 | D83968.1 | Metabolism | Cytochrome | CYP93D | Cytochrome P450 CYP2 subfamily |
| | | | | | P450 | | |
| 526 | Gma.8318.1.A1_at | 0.0544 | BU550469 | No Homology to Known | | | |
| | | | | Proteins | | | |
| 527 | Gma.2912.1.S1_at | 0.0517 | AP117884.1 | Protein Destination & Storage | Storage | | Uncharacterized conserved protein |
| | | | | | Protein | | |
| 528 | Gma.79.1.S1_x_at | 0.0493 | S44202.1 | Unclassified - Protein with | | | |
| | | | | Unknown Function | | | |
| 529 | Gma.14471.1.S1_s_at | 0.0479 | CF921241 | No Homology to Known | | | |
| | | | | Proteins | | | |
| 530 | Gma.1314.1.S1_at | 0.0462 | U63726.1 | Metabolism | | | Gamma-glutamyl hydrolase |
| 531 | GmaAffx.19541.1.S1_s_at | 0.044 | BQ612990 | No Homology to Known | | | |
| | | | | Proteins | | | |
| 532 | GmaAffx.10204.1.S1_at | 0.0421 | BQ298213 | Unclassified - Hypothetical | | | |
| | | | | Protein Supported by cDNA | | | |
| 533 | Gma.5947.1.S1_s_at | 0.0413 | BQ610509 | Disease & Defense | | | |
| 534 | Gma.3774.1.S1_at | 0.0383 | BU550698 | Energy | | | Thioredoxin, nucleoredoxin and related |
| | | | | | | | proteins |
| 535 | Gma.79.1.S1_s_at | 0.0381 | S44202.1 | Unclassified - Protein with | | | |
| | | | | Unknown Function | | | |
| 536 | GmaAffx.68621.1.A1_at | 0.0301 | BI893889 | Unclassified - Protein with | Stress | | |
| | | | | Unknown Function | Induced | | |
| 537 | GmaAffx.85382.1.S1_s_at | 0.0234 | BI945154 | No Homology to Known | | | |
| | | | | Proteins | | | |
| 538 | Gma.3774.2.S1_a_at | 0.0216 | CD393365 | Energy | | | Thioredoxin, nucleoredoxin and related |
| | | | | | | | proteins |
| 539 | Gma.15307.1.S1_at | 0.0127 | U64866.1 | Disease & Defense | | | |

TABLE 7

| | | Fold | | | | | |
|---|---|---|---|---|---|---|---|
| ID | Gene Name | Change | Soybean_Gen Bank_Acc | Functional Category | Sub-Family | Gene Family | Additional Description |
| 1 | Gma.4058.1.S1_at | 29.77 | AW310338 | Transporter | | | Predicted transporter (major |
| | | | | | | | facilitator superfamily) |
| 2 | GmaAffx.19116.1.S1_at | 18.56 | BI968419 | Signal Transduction | Protein | | |
| | | | | | Kinase | | |
| 3 | Gma.10921.1.S1_s_at | 18.02 | BQ611250 | No Homology to Known Proteins | | | |
| 4 | Gma.4071.1.S1_at | 12.74 | BG651692 | Unclassified - Hypothetical Protein | | | |
| | | | | Supported by cDNA | | | |
| 5 | GmaAffx.32345.1.A1_s_at | 12.69 | BI970277 | Cell Structure | | | |
| 6 | GmaAffx.89075.1.S1_s_at | 10.01 | CK606454 | No Homology to Known Proteins | | | |
| 7 | GmaAffx.93260.1.S1_at | 9.745 | CF808985 | Signal Transduction | Protein | | |
| | | | | | Kinase | | |
| 8 | Gma.5689.3.S1_s_at | 8.693 | BE657645 | Protein Destination & Storage | | | Gelatinase A and related matrix |
| | | | | | | | metalloproteases |
| 9 | GmaAffx.50677.1.S1_s_at | 6.936 | BI971744 | No Homology to Known Proteins | | | |
| 10 | Gma.152.1.S1_at | 6.928 | Y10492.1 | Metabolism | Cytochrome | CYP93D | Cytochrome P450 CYP2 subfamily |
| | | | | | P450 | | |
| 11 | Gma.8416.1.S1_at | 6.715 | U13987.1 | Metabolism | | | Cytochrome b5 |
| 11 | Gma.8416.1.S1_at | 6.715 | U13987.1 | Metabolism | | | NADH-cytochrome b-5 reductase |
| 11 | Gma.8416.1.S1_at | 6.715 | U13987.1 | Metabolism | | | Sulfite oxidase, molybdopterin- |
| | | | | | | | binding component |
| 12 | GmaAffx.9866.1.S1_at | 6.693 | BM524432 | Metabolism | | | |
| 13 | Gma.17789.1.S1_at | 6.487 | BG511704 | Unclassified - Hypothetical Protein | | | |
| | | | | Supported by cDNA | | | |
| 14 | Gma.5629.1.S1_at | 6.247 | BQ740550 | Disease & Defense | | | |
| 15 | Gma.320.1.S1_at | 5.971 | AF019116.1 | Disease & Defense | | | |
| 16 | Gma.3804.1.A1_at | 5.568 | AW310249 | Disease & Defense | | | |
| 17 | GmaAffx.11276.1.S1_s_at | 5.327 | AW567677 | Unclassified - Hypothetical Protein | | | |
| | | | | Supported by cDNA | | | |
| 18 | GmaAffx.73185.1.S1_at | 5.042 | BI701903 | No Homology to Known Proteins | | | |
| 19 | GmaAffx.90960.1.A1_s_at | 4.941 | CF806685 | Signal Transduction | Protein | | |
| | | | | | Kinase | | |
| 20 | GmaAffx.87679.1.S1_at | 4.865 | BM523734 | Metabolism | Trehalose | | Trehalose-6-phosphate synthase |
| | | | | | Biosynthesis | | component TPS1 and related subunits |
| 21 | Gma.15523.1.S1_s_at | 4.791 | BE475550 | Cell Structure | | | |
| 22 | Gma.17961.1.S1_at | 4.65 | AW350363 | Protein Destination & Storage | Storage | | |
| | | | | | Protein | | |
| 23 | GmaAffx.10340.1.S1_at | 4.508 | CA938421 | No Homology to Known Proteins | | | |
| 24 | Gma.1248.1.S1_s_at | 4.501 | AJ518837.1 | Metabolism | | | Haloacid dehalogenase-like hydrolase |
| 25 | GmaAffx.93073.1.S1_s_at | 4.465 | CF808798 | Cell Structure | | | |
| 26 | Gma.16832.1.S1_s_at | 4.411 | L22029.1 | No Homology to Known Proteins | | | |
| 27 | Gma.5816.1.S1_at | 4.336 | BI787790 | Unclassified - Hypothetical Protein | | | Aldehyde reductase |
| | | | | Supported by cDNA | | | |
| 28 | Gma.3888.2.S1_at | 4.335 | BM139532 | Unclassified - Hypothetical Protein | | | Aldehyde reductase |
| | | | | NOT Supported by cDNA | | | |
| 29 | GmaAffx.87679.2.S1_s_at | 4.332 | AW102083 | Metabolism | Trehalose | | Trehalose-6-phosphate synthase |
| | | | | | Biosynthesis | | component TPS1 and related subunits |
| 30 | GmaAffx.18566.1.A1_at | 4.291 | BU550417 | Unclassified - Hypothetical Protein | | | |
| | | | | NOT Supported by cDNA | | | |
| 31 | GmaAffx.93541.2.S1_at | 4.208 | CF805889 | Protein Destination & Storage | | | Gelatinase A and related matrix |
| | | | | | | | metalloproteases |
| 32 | Gma.1221.1.S1_s_at | 4.204 | L23854.1 | Metabolism | | | Cytochrome b5 |
| 32 | Gma.1221.1.S1_s_at | 4.204 | L23854.1 | Metabolism | | | NADH-cytochrome b-5 reductase |
| 32 | Gma.1221.1.S1_s_at | 4.204 | L23854.1 | Metabolism | | | Sulfite oxidase, molybdopterin- |
| | | | | | | | binding component |
| 33 | Gma.16425.3.S1_s_at | 4.126 | AW508123 | Unclassified - Protein with | | | Copper chaperone |
| | | | | Unknown Function | | | |
| 34 | Gma.16425.2.S1_s_at | 4.078 | AW099501 | Unclassified - Hypothetical Protein | | | Copper chaperone |
| | | | | NOT Supported by cDNA | | | |
| 35 | Gma.16308.1.S1_s_at | 4.027 | U64916.1 | Unclassified - Hypothetical Protein | | | Copper chaperone |
| | | | | NOT Supported by cDNA | | | |
| 36 | GmaAffx.93529.1.S1_s_at | 3.791 | CF809254 | No Homology to Known Proteins | | | |
| 37 | GmaAffx.10340.1.S1_x_at | 3.77 | CA938421 | No Homology to Known Proteins | | | |
| 38 | GmaAffx.83638.1.S1_at | 3.768 | BU549896 | Unclassified - Hypothetical Protein | | | |
| | | | | NOT Supported by cDNA | | | |
| 39 | GmaAffx.9605.1.S1_at | 3.734 | BM091603 | No Homology to Known Proteins | | | |
| 40 | GmaAffx.70875.1.S1_s_at | 3.656 | AW100693 | No Homology to Known Proteins | | | |
| 41 | Gma.3405.1.S1_s_at | 3.606 | AW350922 | Metabolism | | | Branched chain aminotransferase |
| | | | | | | | BCAT1, pyridoxal phosphate |
| | | | | | | | enzymes type IV superfamily |
| 42 | Gma.6555.1.S1_at | 3.568 | BM954521 | Unclassified - Hypothetical Protein | | | |
| | | | | Supported by cDNA | | | |
| 43 | Gma.4701.2.A1_a_at | 3.545 | BM526048 | Energy | | | Thioredoxin |
| 44 | Gma.1400.2.S1_at | 3.516 | BG510739 | Signal Transduction | Protein | AtCIPKs | Serine/threonine protein kinase |
| | | | | | Kinase | | |
| 45 | GmaAffx.37477.1.S1_s_at | 3.499 | BQ081355 | Metabolism | | | Branched chain aminotransferase |
| | | | | | | | BCAT1, pyridoxal phosphate |
| | | | | | | | enzymes type IV superfamily |
| 46 | GmaAffx.67477.2.S1_at | 3.453 | AW509184 | Cell Structure | | | |
| 47 | GmaAffx.91346.1.S1_x_at | 3.41 | CF807071 | Unclassified - Hypothetical Protein | | | |
| | | | | Supported by cDNA | | | |
| 48 | GmaAffx.11858.1.A1_s_at | 3.402 | AW351023 | Metabolism | Phospholipid | | Glycosylphosphatidylinositol-specific |
| | | | | | Biosynthesis | | phospholipase C |
| 49 | GmaAffx.92537.1.S1_at | 3.401 | CF808262 | Energy | | | Thioredoxin |
| 50 | Gma.3639.1.S1_at | 3.4 | AB083030.1 | Protein Destination & Storage | Storage | | |
| | | | | | Protein | | |
| 51 | Gma.6288.1.S1_at | 3.388 | AW348556 | Transporter | | | Aquaporin (major intrinsic protein |
| | | | | | | | family) |
| 52 | Gma.5483.1.S1_s_at | 3.362 | CD414581 | Transcription | Transcription | Basic | |
| | | | | | Factor | Leucine | |
| | | | | | | Zipper | |
| | | | | | | (bZlP) | |
| 53 | GmaAffx.85409.1.S1_at | 3.337 | BF598665 | Unclassified - Hypothetical Protein | | | Copper chaperone |
| | | | | NOT Supported by cDNA | | | |
| 54 | Gma.17559.1.S1_at | 3.31 | BG508388 | No Homology to Known Proteins | | | |
| 55 | GmaAffx.87812.2.S1_at | 3.31 | BI971676 | Signal Transduction | Protein | | |
| | | | | | Kinase | | |
| 56 | GmaAffx.87526.1.S1_at | 3.286 | BM954581 | Unclassified - Hypothetical Protein | | | |
| | | | | Supported by cDNA | | | |
| 57 | Gma.13447.1.A1_at | 3.285 | CD398305 | No Homology to Known Proteins | | | |
| 58 | GmaAffx.93462.1.S1_s_at | 3.272 | CF809187 | Signal Transduction | Protein | AtCIPKs | Serine/threonine protein kinase |
| | | | | | Kinase | | |
| 59 | GmaAffx.24352.1.S1_at | 3.243 | BU544287 | No Homology to Known Proteins | | | |
| 60 | Gma.7332.1.S1_at | 3.196 | BI945636 | Unclassified - Hypothetical Protein | | | |
| | | | | Supported by cDNA | | | |
| 61 | GmaAffx.81930.1.S1_at | 3.194 | BG789998 | Unclassified - Hypothetical Protein | | | |
| | | | | Supported by cDNA | | | |
| 62 | Gma.8896.1.S1_at | 3.179 | BQ252812 | Transcription | Transcription | AP2/EREBP | |
| | | | | | Factor | | |
| 63 | Gma.14471.2.S1_at | 3.168 | CD408292 | Transporter | | | Aquaporin (major intrinsic protein |
| | | | | | | | family) |
| 64 | Gma.3149.1.S1_a_at | 3.154 | BU544575 | No Homology to Known Proteins | | | |
| 65 | GmaAffx.68088.1.S1_at | 3.1 | BG507880 | Signal Transduction | Protein | | |
| | | | | | Kinase | | |
| 66 | GmaAffx.3905.1.S1_s_at | 3.085 | CA818952 | Unclassified - Protein with | | | |
| | | | | Unknown Function | | | |
| 67 | GmaAffx.54265.1.S1_s_at | 3.068 | BU546096 | Signal Transduction | Protein | | |
| | | | | | Kinase | | |
| 68 | Gma.4226.2.S1_at | 3.037 | BM568170 | Unclassified - Hypothetical Protein | | | |
| | | | | Supported by cDNA | | | |
| 69 | Gma.8301.2.S1_at | 3.014 | BU544524 | Signal Transduction | Calcium | | |
| | | | | | Binding | | |
| 70 | GmaAffx.62542.1.S1_at | 2.983 | BE659136 | Unclassified - Hypothetical Protein | | | |
| | | | | NOT Supported by cDNA | | | |
| 71 | GmaAffx.3905.3.S1_at | 2.969 | BG047142 | Unclassified - Protein with | | | |
| | | | | Unknown Function | | | |
| 72 | GmaAffx.31180.1.A1_at | 2.939 | BE820254 | No Homology to Known Proteins | | | |
| 73 | GmaAffx.85264.1.S1_at | 2.932 | BE058913 | Metabolism | | | |
| 74 | GmaAffx.90947.1.S1_at | 2.927 | CF806672 | Unclassified - Protein with | | | |
| | | | | Unknown Function | | | |
| 75 | GmaAffx.2743.1.S1_s_at | 2.913 | BM568087 | Signal Transduction | Protein | | |
| | | | | | Kinase | | |
| 76 | Gma.43.1.A1_at | 2.884 | L12258.1 | Transporter | | | Aquaporin (major intrinsic protein |
| | | | | | | | family) |
| 77 | GmaAffx.40860.2.S1_at | 2.85 | BG509282 | Disease & Defense | | | |
| 78 | Gma.2548.1.S1_at | 2.841 | BE821472 | Unclassified - Hypothetical Protein | | | |
| | | | | Supported by cDNA | | | |
| 79 | GmaAffx.43291.1.S1_at | 2.782 | AI442332 | Metabolism | | | Lipid phosphate phosphatase and |
| | | | | | | | related enzymes of the PAP2 family |
| 80 | Gma.4526.1.S1_at | 2.781 | BI967711 | Transcription | Transcription | Zinc Finger | FOG: Zn-finger |
| | | | | | Factor | | |
| 81 | GmaAffx.10773.1.S1_at | 2.769 | BU926045 | Unclassified - Hypothetical Protein | | | |
| | | | | Supported by cDNA | | | |
| 82 | GmaAffx.85374.1.S1_at | 2.76 | BI317002 | Signal Transduction | Protein | | |
| | | | | | Kinase | | |
| 83 | Gma.11259.1.S1_at | 2.733 | AI442316 | Unclassified - Hypothetical Protein | | | |
| | | | | Supported by cDNA | | | |
| 84 | GmaAffx.4905.1.S1_at | 2.73 | BQ453808 | Metabolism | | | Predicted lipase |
| 85 | Gma.10756.1.S1_s_at | 2.682 | BE822092 | Unclassified - Hypothetical Protein | | | |
| | | | | Supported by cDNA | | | |
| 86 | Gma.4051.1.S1_at | 2.682 | L22162.1 | Cell Structure | | | |
| 87 | Gma.7474.1.A1_at | 2.672 | BU547968 | Unclassified - Hypothetical Protein | | | |
| | | | | Supported by cDNA | | | |
| 88 | Gma.17605.1.S1_at | 2.637 | BI968208 | Secondary Metabolism | Flavonoid | | |
| | | | | | Biosynthesis | | |
| 89 | Gma.6266.1.A1_at | 2.593 | CD413418 | Unclassified - Protein with | | | |
| | | | | Unknown Function | | | |
| 90 | Gma.340.1.S1_at | 2.59 | AW350127 | Cell Structure | | | |
| 91 | Gma.13361.1.S1_at | 2.588 | AW309932 | Unclassified - Protein with | | | |
| | | | | Unknown Function | | | |
| 92 | GmaAffx.16502.1.A1_at | 2.585 | BE659118 | No Homology to Known Proteins | | | |
| 93 | Gma.4336.1.S1_at | 2.564 | BQ079907 | Unclassified - Hypothetical Protein | | | |
| | | | | NOT Supported by cDNA | | | |
| 94 | GmaAffx.81783.1.S1_at | 2.559 | AW101961 | Signal Transduction | Protein | | Serine/threonine protein kinase |
| | | | | | Kinase | | |
| 95 | GmaAffx.8098.1.S1_at | 2.546 | AW156410 | No Homology to Known Proteins | | | |
| 96 | Gma.184.1.S1_at | 2.534 | AW101261 | Unclassified - Hypothetical Protein | | | |
| | | | | Supported by cDNA | | | |
| 97 | GmaAffx.9400.1.S1_at | 2.528 | BI893703 | Signal Transduction | ProteinKinase | | Serine/threonine protein kinase |
| 98 | Gma.3522.1.S1_at | 2.526 | BM140209 | Unclassified - Hypothetical Protein | | | |
| | | | | Supported by cDNA | | | |
| 99 | GmaAffx.40453.1.S1_at | 2.501 | BE658683 | No Homology to Known Proteins | | | |
| 100 | GmaAffx.18518.1.S1_at | 2.498 | BI700329 | Cell Structure | | | |
| 101 | Gma.12356.1.A1_at | 2.497 | CD397798 | Unclassified - Hypothetical Protein | | | |
| | | | | NOT Supported by cDNA | | | |
| 102 | Gma.9141.1.A1_at | 2.484 | AW310721 | Secondary Metabolism | | | |
| 103 | GmaAffx.87812.1.S1_at | 2.455 | AW831390 | Signal Transduction | Protein | | |
| | | | | | Kinase | | |
| 104 | GmaAffx.87547.2.S1_at | 2.45 | BQ080473 | Cell Structure | | | Beta-glucosidase, lactase |
| | | | | | | | phlorizinhydrolase, and related |
| | | | | | | | proteins |
| 105 | GmaAffx.91346.1.S1_at | 2.437 | CF807071 | Unclassified - Hypothetical Protein | | | |
| | | | | Supported by cDNA | | | |
| 106 | GmaAffx.65829.1.A1_at | 2.428 | CD392418 | Transcription | Transcription | AP2/EREBP | |
| | | | | | Factor | | |
| 107 | GmaAffx.78636.1.S1_at | 2.428 | BQ627652 | No Homology to Known Proteins | | | |
| 108 | GmaAffx.5264.1.S1_s_at | 2.415 | BM521512 | Disease & Defense | | | |
| 109 | GmaAffx.89512.1.A1_at | 2.403 | CK606530 | No Homology to Known Proteins | | | |
| 110 | Gma.368.1.S1_at | 2.387 | AW349594 | No Homology to Known Proteins | | | |
| 111 | GmaAffx.61907.1.S1_at | 2.376 | CA819279 | No Homology to Known Proteins | | | |
| 112 | GmaAffx.83598.1.S1_at | 2.373 | BU964918 | Transcription | Transcription | AP2/EREBP | |
| | | | | | Factor | | |
| 113 | Gma.4396.1.S1_at | 2.368 | BQ081294 | No Homology to Known Proteins | | | |
| 114 | Gma.17887.1.S1_s_at | 2.367 | BG042115 | Unclassified - Hypothetical Protein | | | |
| | | | | Supported by cDNA | | | |
| 115 | Gma.1521.1.S1_at | 2.363 | CD394504 | Cell Structure | | | Beta tubulin |
| 116 | Gma.6343.1.S1_at | 2.356 | AI442963 | Unclassified - Hypothetical Protein | | | |
| | | | | NOT Supported by cDNA | | | |
| 117 | GmaAffx.55404.1.S1_at | 2.351 | BM271530 | No Homology to Known Proteins | | | |
| 118 | GmaAffx.50347.1.S1_at | 2.342 | BQ740576 | Signal Transduction | Protein | | |
| | | | | | Kinase | | |
| 119 | GmaAffx.21211.1.S1_at | 2.335 | BU550606 | Protein Destination & Storage | | | Molecular chaperone (HSP90 family) |
| 120 | Gma.2488.1.S1_at | 2.327 | CD406079 | Protein Destination & Storage | | | Serine carboxypeptidases (lysosomal |
| | | | | | | | cathepsin A) |
| 121 | Gma.3104.1.A1_at | 2.314 | BG839320 | Signal Transduction | Protein | | Serine/threonine protein kinase |
| | | | | | Kinase | | |
| 122 | GmaAffx.12370.1.S1_at | 2.312 | BE059411 | Cell Growth & Division | | | Microtubule-associated protein |
| | | | | | | | essential for anaphase spindle |
| | | | | | | | elongation |
| 123 | Gma.17300.1.A1_at | 2.289 | AW350946 | Unclassified - Hypothetical Protein | | | |
| | | | | NOT Supported by cDNA | | | |
| 124 | Gma.16913.1.S1_s_at | 2.287 | BI426712 | Unclassified - Protein with | | | |
| | | | | Unknown Function | | | |
| 125 | GmaAffx.33259.1.S1_at | 2.272 | BQ785322 | Unclassified - Hypothetical Protein | | | |
| | | | | Supported by cDNA | | | |
| 126 | GmaAffx.85255.1.S1_at | 2.265 | BE059755 | No Homology to Known Proteins | | | |
| 127 | Gma.3969.2.S1_at | 2.246 | CD412522 | Energy | | | |
| 128 | Gma.666.1.S1_at | 2.239 | AW310734 | Unclassified - Protein with | Hormone | | Voltage-gated shaker-like K+ |
| | | | | Unknown Function | Induced | | channel, subunit beta/KCNAB |
| 129 | GmaAffx.60984.1.S1_at | 2.234 | BU765206 | Transporter | | | |
| 130 | Gma.3555.1.S1_at | 2.23 | BU764259 | Unclassified - Hypothetical Protein | | | |
| | | | | NOT Supported by cDNA | | | |
| 131 | GmaAffx.56917.1.S1_at | 2.227 | AI1441130 | Signal Transduction | Protein | | Serine/threonine protein kinase |
| | | | | | Kinase | | |
| 132 | GmaAffx.86059.1.S1_at | 2.206 | BF598759 | No Homology to Known Proteins | | | |
| 133 | GmaAffx.47891.1.S1_s_at | 2.197 | BQ785880 | Cell Structure | | | |
| 134 | GmaAffx.37574.1.S1_s_at | 2.196 | BQ298504 | Secondary Metabolism | | | Hydroxyindole-O-methyltransferase |
| | | | | | | | and related SAM-dependent |
| | | | | | | | methyltransferases |
| 135 | GmaAffx.13625.1.A1_at | 2.195 | BU547925 | No Homology to Known Proteins | | | |
| 136 | Gma.3777.1.S1_a_at | 2.191 | CD399954 | Unclassified - Hypothetical Protein | | | |
| | | | | Supported by cDNA | | | |
| 137 | Gma.11119.2.S1_s_at | 2.19 | CF920743 | Disease & Defense | | | |
| 138 | GmaAffx.84266.1.S1_at | 2.19 | AI495798 | Signal Transduction | Protein | AtCIPKs | Serine/threonine protein kinase |
| | | | | | Kinase | | |
| 139 | Gma.10573.3.S1_at | 2.185 | CA938507 | Transcription | Transcription | Zinc Finger | GATA-4/5/6 transcription factors |
| | | | | | Factor | | |
| 140 | GmaAffx.93561.1.S1_at | 2.178 | CF807977 | Metabolism | | | Branched chain aminotransferase |
| | | | | | | | BCAT1, pyridoxal phosphate |
| | | | | | | | enzymes type IV superfamily |
| 141 | Gma.3777.1.S1_x_at | 2.178 | CD399954 | Unclassified - Hypothetical Protein | | | |
| | | | | Supported by cDNA | | | |
| 142 | Gma.5950.1.S1_s_at | 2.154 | BQ610563 | Disease & Defense | | | |
| 143 | Gma.3751.1.S1_at | 2.134 | AW350303 | Unclassified - Hypothetical Protein | | | |
| | | | | NOT Supported by cDNA | | | |
| 144 | GmaAffx.93214.1.S1_at | 2.117 | CF808939 | No Homology to Known Proteins | | | |
| 145 | Gma.10334.1.S1_at | 2.104 | AW234479 | Unclassified - Hypothetical Protein | | | |
| | | | | NOT Supported by cDNA | | | |
| 146 | GmaAffx.78599.1.A1_at | 2.1 | CA784082 | No Homology to Known Proteins | | | |
| 147 | GmaAffx.10491.1.S1_at | 2.082 | AW598141 | No Homology to Known Proteins | | | |
| 148 | GmaAffx.77804.1.A1_at | 2.079 | BI968697 | No Homology to Known Proteins | | | |
| 149 | Gma.4437.1.S1_s_at | 2.079 | BQ628831 | Secondary Metabolism | Flavonoid | | Flavonol reductase/cinnamoyl-CoA |
| | | | | | Biosynthesis | | reductase |
| 150 | Gma.6650.1.S1_at | 2.075 | BQ629283 | Unclassified - Protein with | | | |
| | | | | Unknown Function | | | |
| 151 | GmaAffx.90042.1.S1_at | 2.073 | CF805767 | Signal Transduction | Calcium | | Calmodulin and related proteins (EF- |
| | | | | | Binding | | Hand superfamily) |
| 152 | GmaAffx.9618.1.S1_at | 2.067 | BM092432 | No Homology to Known Proteins | | | |
| 153 | Gma.17816.1.A1_at | 2.059 | BI970976 | Disease & Defense | | | |
| 154 | Gma.2475.1.S1_at | 2.043 | BM522941 | Cell Structure | | | |
| 155 | Gma.18084.1.S1_at | 2.026 | BI317557 | Disease & Defense | | | Ferric reductase, NADH/NADPH |
| | | | | | | | oxidase and related proteins |
| 156 | Gma.11257.1.S1_at | 2.016 | AW310476 | Cell Structure | | | |
| 157 | Gma.6719.3.S1_s_at | 2.012 | AW164562 | Unclassified - Hypothetical Protein | | | |
| | | | | NOT Supported by cDNA | | | |
| 158 | Gma.3189.2.S1_at | 2.005 | BG511492 | Signal Transduction | MAPKK | | Mitogen-activated protein kinase |
| | | | | | | | kinase (MAP2K) |
| 159 | GmaAffx.74847.1.S1_at | 0.5 | BG507699 | Metabolism | | | |
| 160 | Gma.6761.1.A1_at | 0.5 | BQ453407 | Cell Structure | | | Beta-glucosidase, lactase |
| | | | | | | | phlorizinhydrolase, and related |
| | | | | | | | proteins |
| 161 | GmaAffx.68754.1.S1_at | 0.499 | BE474746 | Secondary Metabolism | | | Hydroxyindole-O-methyltransferase |
| | | | | | | | and related SAM-dependent |
| | | | | | | | methyltransferases |
| 162 | GmaAffx.84566.3.S1_s_at | 0.499 | BG045916 | Transcription | Transcription | MYB | Transcription factor, Myb |
| | | | | | Factor | | superfamily |
| 163 | Gma.14338.1.A1_at | 0.499 | CF923111 | Protein Destination & Storage | Storage | | |
| | | | | | Protein | | |
| 164 | GmaAffx.86247.1.S1_s_at | 0.499 | BG238674 | Unclassified - Hypothetical Protein | | | |
| | | | | Supported by cDNA | | | |
| 165 | Gma.4414.1.A1_at | 0.498 | BQ081678 | No Homology to Known Proteins | | | |
| 166 | Gma.8113.1.A1_at | 0.497 | BU548342 | No Homology to Known Proteins | | | |
| 167 | GmaAffx.15862.1.S1_at | 0.497 | BM092634 | Cell Structure | | | |
| 168 | GmaAffx.52963.1.S1_at | 0.496 | AW310673 | Unclassified - Protein with | | | |
| | | | | Unknown Function | | | |
| 169 | GmaAffx.74674.1.S1_s_at | 0.496 | BU965464 | Transporter | Auxin | | |
| | | | | | Transport | | |
| 170 | GmaAffx.93220.1.S1_at | 0.496 | CF808945 | No Homology to Known Proteins | | | |
| 171 | GmaAffx.24350.1.A1_at | 0.495 | BU544060 | No Homology to Known Proteins | | | |
| 172 | GmaAffx.15983.2.S1_s_at | 0.495 | AW279540 | Signal Transduction | Protein | | Serine/threonine protein phosphatase |
| | | | | | Phosphatase | | |
| 173 | GmaAffx.35729.1.S1_s_at | 0.495 | BE806047 | Unclassified - Hypothetical Protein | | | Predicted E3 ubiquitin ligase |
| | | | | Supported by cDNA | | | |
| 174 | GmaAffx.90069.1.S1_s_at | 0.494 | CF805794 | Unclassified - Hypothetical Protein | | | Leucine rich repeat proteins, some |
| | | | | Supported by cDNA | | | proteins contain F-box |
| 175 | GmaAffx.83226.1.S1_at | 0.494 | CA852250 | Transcription | Transcription | Unclassified | |
| | | | | | Factor | | |
| 176 | Gma.1622.1.A1_s_at | 0.493 | BU090523 | Signal Transduction | Protein | | FOG: Transposon-encoded proteins |
| | | | | | Kinase | | with TYA, reverse transcriptase, |
| | | | | | | | integrase domains in various |
| | | | | | | | combinations |
| 177 | GmaAffx.85528.1.S1_at | 0.493 | BE608070 | Unclassified - Hypothetical Protein | | | FOG: PPR repeat |
| | | | | Supported by cDNA | | | |
| 178 | Gma.2252.1.S1_at | 0.492 | CD406598 | Unclassified - Hypothetical Protein | | | |
| | | | | Supported by cDNA | | | |
| 179 | Gma.9259.1.S1_at | 0.492 | CA782161 | Transporter | | | |
| 180 | GmaAffx.92927.1.S1_at | 0.491 | CF805992 | No Homology to Known Proteins | | | |
| 181 | Gma.6895.1.S1_a_at | 0.491 | BU545788 | Metabolism | | | Alanine-glyoxylate aminotransferase |
| | | | | | | | AGT2 |
| 182 | GmaAffx.57878.2.S1_at | 0.491 | BE610836 | Cell Structure | | | |
| 183 | Gma.6375.1.S1_at | 0.489 | BQ610190 | Unclassified - Hypothetical Protein | | | AAA+-type ATPase |
| | | | | NOT Supported by cDNA | | | |
| 184 | Gma.3332.1.S1_at | 0.489 | BQ298780 | Protein Destination & Storage | | | |
| 185 | GmaAffx.92536.1.S1_s_at | 0.489 | CF808261 | Secondary Metabolism | Ethylene | | Iron/ascorbate family oxidoreductases |
| | | | | | Biosynthesis | | |
| 186 | Gma.17638.2.S1_at | 0.489 | BI786260 | Transcription | Transcription | Unclassified | GATA-4/5/6 transcription factors |
| | | | | | Factor | | |
| 187 | Gma.17610.1.S1_x_at | 0.488 | U50075.1 | Metabolism | | | |
| 188 | Gma.5701.3.S1_at | 0.488 | CA852693 | No Homology to Known Proteins | | | |
| 189 | GmaAffx.81999.1.S1_at | 0.487 | AI416637 | Transcription | Transcription | Homeodomain | Transcription factor MEIS1 and |
| | | | | | Factor | | related HOX domain proteins |
| 190 | GmaAffx.83880.1.A1_at | 0.486 | BE822640 | Disease & Defense | | | |
| 191 | GmaAffx.55657.1.S1_at | 0.486 | BM885600 | No Homology to Known Proteins | | | |
| 192 | GmaAffx.57967.1.S1_at | 0.486 | AW102170 | Protein Destination & Storage | | | |
| 193 | Gma.12987.1.S1_at | 0.485 | CD392747 | Transcription | Transcription | Basic Helix- | |
| | | | | | Factor | Loop-Helix | |
| | | | | | | (bHLH) | |
| 194 | Gma.13218.1.A1_s_at | 0.484 | CD391892 | No Homology to Known Proteins | | | |
| 195 | GmaAffx.60479.1.S1_at | 0.484 | BG726023 | Transcription | Transcription | Homeodomain | Transcription factor MEIS1 and |
| | | | | | Factor | | related HOX domain proteins |
| 196 | GmaAffx.50613.1.A1_at | 0.484 | BI967418 | No Homology to Known Proteins | | | |
| 197 | Gma.3589.1.S1_a_at | 0.483 | AW101438 | Secondary Metabolism | GA | | Iron/ascorbate family oxidoreductases |
| | | | | | Biosynthesis | | |
| 198 | Gma.1573.2.S1_a_at | 0.483 | BI974098 | Metabolism | | | |
| 199 | GmaAffx.23789.1.S1_at | 0.482 | BU551014 | Unclassified - Hypothetical Protein | | | |
| | | | | Supported by cDNA | | | |
| 200 | Gma.221.1.S1_at | 0.481 | U41475.1 | Metabolism | | | Phosphoinositide-specific |
| | | | | | | | phospholipase C |
| 201 | GmaAffx.55124.1.S1_at | 0.481 | BI970821 | Unclassified - Hypothetical Protein | | | Metacaspase involved in regulation of |
| | | | | Supported by cDNA | | | apoptosis |
| 202 | Gma.6643.1.A1_at | 0.481 | BE823205 | Secondary Metabolism | | | Sulfotransferase |
| 203 | Gma.17610.1.S1_at | 0.481 | U50075.1 | Metabolism | | | |
| 204 | GmaAffx.13039.1.A1_at | 0.481 | BF219584 | Secondary Metabolism | | | |
| 205 | GmaAffx.53845.1.S1_at | 0.48 | BG406552 | Transporter | | | Sulfate/bicarbonate/oxalate exchanger |
| | | | | | | | SAT-1 and related transporters |
| | | | | | | | (SLC26 family) |
| 206 | Gma.5294.1.S1_at | 0.479 | BE658252 | Unclassified - Hypothetical Protein | | | |
| | | | | Supported by cDNA | | | |
| 207 | GmaAffx.73857.1.S1_at | 0.479 | BM885538 | Unclassified - Hypothetical Protein | | | |
| | | | | Supported by cDNA | | | |
| 208 | Gma.17798.2.S1_at | 0.479 | CD412748 | No Homology to Known Proteins | | | |
| 209 | Gma.13273.1.A1_at | 0.478 | CD397107 | Unclassified - Hypothetical Protein | | | |
| | | | | Supported by cDNA | | | |
| 210 | GmaAffx.47451.1.S1_at | 0.478 | CA799081 | Unclassified - Hypothetical Protein | | | |
| | | | | NOT Supported by cDNA | | | |
| 211 | GmaAffx.90234.1.S1_at | 0.477 | CF805959 | Transcription | Transcription | Zinc Finger | FOG: Zn-finger |
| | | | | | Factor | | |
| 212 | GmaAffx.6635.1.S1_at | 0.477 | CD412718 | Disease & Defense | | | |
| 213 | GmaAffx.14141.1.A1_at | 0.476 | BI973009 | No Homology to Known Proteins | | | |
| 214 | Gma.6129.2.S1_at | 0.475 | AW 186006 | Protein Synthesis | | | Translation initiation factor 5A (elF- |
| | | | | | | | 5A) |
| 215 | GmaAffx.499.1.S1_at | 0.475 | BE058375 | Transcription | Transcription | MYB | Transcription factor, Myb |
| | | | | | Factor | | superfamily |
| 216 | Gma.2565.1.S1_at | 0.475 | BG652533 | Unclassified - Hypothetical Protein | | | |
| | | | | Supported by cDNA | | | |
| 217 | Gma.5331.1.S1_a_at | 0.475 | BI892702 | Transcription | Transcription | NAC Domain | |
| | | | | | Factor | | |
| 218 | GmaAffx.84338.1.S1_at | 0.474 | AI856789 | Unclassified - Hypothetical Protein | | | Predicted E3 ubiquitin ligase |
| | | | | Supported by cDNA | | | |
| 219 | Gma.3519.1.S1_at | 0.474 | CD403581 | Unclassified - Hypothetical Protein | | | |
| | | | | Supported by cDNA | | | |
| 220 | Gma.13035.1.S1_at | 0.474 | CD396536 | No Homology to Known Proteins | | | |
| 221 | Gma.17502.2.S1_x_at | 0.473 | BG046201 | Transcription | Transcription | Unclassified | Zuotin and related molecular |
| | | | | | Factor | | chaperones (DnaJ superfamily), |
| | | | | | | | contains DNA-binding domains |
| 222 | Gma.6310.1.S1_at | 0.472 | BQ612831 | Signal Transduction | MAPKKK | MEKK | MEKK and related serine/threonine |
| | | | | | | | protein kinases |
| 223 | GmaAffx.73059.1.S1_at | 0.472 | BI426008 | Unclassified - Hypothetical Protein | | | Predicted dehydrogenase |
| | | | | NOT Supported by cDNA | | | |
| 224 | GmaAffx.93430.1.S1_s_at | 0.47 | CF809155 | Disease & Defense | | | Purple (tartrate-resistant) acid |
| | | | | | | | phosphatase |
| 225 | GmaAffx.15471.1.S1_at | 0.47 | BQ611747 | Transcription | Transcription | MYB | Transcription factor, Myb |
| | | | | | Factor | | superfamily |
| 226 | GmaAffx.74256.1.S1_at | 0.469 | BE057293 | No Homology to Known Proteins | | | |
| 227 | GmaAffx.18376.1.A1_at | 0.469 | BI321742 | Protein Destination & Storage | | | Ubiquitin-protein ligase |
| 228 | GmaAffx.69198.1.S1_at | 0.468 | BM526951 | Unclassified - Hypothetical Protein | | | Leucine rich repeat proteins, some |
| | | | | Supported by cDNA | | | proteins contain F-box |
| 229 | GmaAffx.33527.1.S1_at | 0.468 | BU761195 | No Homology to Known Proteins | | | |
| 230 | Gma.13800.1.A1_at | 0.468 | CD405906 | Unclassified - Hypothetical Protein | | | |
| | | | | NOT Supported by cDNA | | | |
| 231 | GmaAffx.78561.1.S1_at | 0.467 | BQ452483 | Metabolism | | | UDP-glucuronosyl and UDP-glucosyl |
| | | | | | | | transferase |
| 232 | Gma.7040.1.A1_at | 0.467 | BE346424 | Transcription | Transcription | AP2/EREBP | |
| | | | | | Factor | | |
| 233 | GmaAffx.26320.1.S1_at | 0.466 | BE440416 | Unclassified - Hypothetical Protein | | | |
| | | | | Supported by cDNA | | | |
| 234 | GmaAffx.494.1.S1_at | 0.464 | AI938330 | Cell Structure | | | Chitinase |
| 235 | Gma.4095.1.S1_at | 0.463 | AW350037 | Unclassified - Hypothetical Protein | | | |
| | | | | Supported by cDNA | | | |
| 236 | GmaAffx.78297.1.S1_at | 0.463 | BM731185 | Unclassified - Hypothetical Protein | | | FOG: Predicted E3 ubiquitin ligase |
| | | | | Supported by cDNA | | | |
| 237 | GmaAffx.93116.1.S1_at | 0.463 | CF806290 | Disease & Defense | | | |
| 238 | GmaAffx.47288.1.S1_at | 0.461 | BU764979 | Unclassified - Hypothetical Protein | | | GTPase Rab18, small G protein |
| | | | | Supported by cDNA | | | superfamily |
| 239 | GmaAffx.15204.1.A1_at | 0.461 | BU549835 | No Homology to Known Proteins | | | |
| 240 | Gma.6213.1.S1_at | 0.461 | CD401652 | Protein Destination & Storage | | | Glutathione S-transferase |
| 241 | GmaAffx.35908.1.S1_at | 0.46 | BF598958 | No Homology to Known Proteins | | | |
| 242 | Gma.2780.1.A1_at | 0.458 | AI988066 | Unclassified - Hypothetical Protein | | | UDP-galactose transporter related |
| | | | | NOT Supported by cDNA | | | protein |
| 243 | Gma.3578.1.S1_s_at | 0.458 | AW156522 | Disease & Defense | | | Acyl-CoA synthetase |
| 244 | GmaAffx.50673.2.S1_s_at | 0.458 | BI787147 | Transcription | Transcription | NAC Domain | |
| | | | | | Factor | | |
| 245 | GmaAffx.23292.1.A1_at | 0.457 | BI967795 | No Homology to Known Proteins | | | |
| 246 | GmaAffx.91143.1.S1_at | 0.457 | CF806868 | Signal Transduction | Unclassified | | |
| 247 | Gma.1619.1.S1_at | 0.457 | AW472195 | Unclassified - Hypothetical Protein | | | |
| | | | | Supported by cDNA | | | |
| 248 | Gma.17070.1.S1_at | 0.456 | BE822463 | Disease & Defense | | | O-methyltransferase |
| 249 | Gma.11026.2.S1_s_at | 0.455 | BQ080041 | Disease & Defense | | | |
| 250 | GmaAffx.90899.1.S1_at | 0.454 | CF806624 | Unclassified - Hypothetical Protein | | | |
| | | | | NOT Supported by cDNA | | | |
| 251 | Gma.8490.1.S1_at | 0.454 | AB030492.1 | Metabolism | | | Protein involved in thiamine |
| | | | | | | | biosynthesis and DNA damage |
| | | | | | | | tolerance |
| 252 | GmaAffx.84964.1.S1_at | 0.453 | AW598267 | No Homology to Known Proteins | | | |
| 253 | GmaAffx.76699.1.S1_at | 0.451 | AW760679 | Transcription | Transcription | Basic Helix- | |
| | | | | | Factor | Loop-Helix | |
| | | | | | | (bHLH) | |
| 254 | Gma.8370.1.S1_at | 0.45 | BQ454087 | Signal Transduction | Protein | | Serine/threonine protein phosphatase |
| | | | | | Phosphatase | | |
| 255 | GmaAffx.41990.1.S1_at | 0.449 | CA936708 | No Homology to Known Proteins | | | |
| 256 | GmaAffx.32700.1.S1_at | 0.449 | BM522940 | No Homology to Known Proteins | | | |
| 257 | Gma.9205.1.S1_at | 0.447 | BM528605 | Unclassified - Hypothetical Protein | | | |
| | | | | Supported by cDNA | | | |
| 258 | Gma.4400.1.S1_at | 0.447 | BI317776 | Transcription | Transcription | WRKY | |
| | | | | | Factor | | |
| 259 | GmaAffx.45003.1.S1_at | 0.445 | BI968048 | Unclassified - Hypothetical Protein | | | Uncharacterized conserved protein |
| | | | | Supported by cDNA | | | |
| 260 | Gma.6033.2.S1_at | 0.444 | AI416728 | Unclassified - Hypothetical Protein | | | |
| | | | | Supported by cDNA | | | |
| 261 | GmaAffx.73452.2.S1_at | 0.444 | BM085321 | Signal Transduction | Phytochrome | | eIF-2alpha kinase PEK/EIF2AK3 |
| 262 | Gma.1929.1.S1_at | 0.443 | BU083650 | Transcription | Transcription | Homeodomain | Transcription factor HEX, contains |
| | | | | | Factor | | HOX and HALZ domains |
| 263 | GmaAffx.49777.1.S1_at | 0.443 | BG047001 | No Homology to Known Proteins | | | |
| 264 | Gma.11205.1.S1_a_at | 0.442 | BF066553 | Metabolism | | | Transketolase |
| 265 | GmaAffx.34798.1.S1_at | 0.442 | BM143105 | Unclassified - Hypothetical Protein | | | |
| | | | | Supported by cDNA | | | |
| 266 | GmaAffx.69369.1.S1_at | 0.442 | BG511072 | Unclassified - Hypothetical Protein | | | Galactosyltransferases |
| | | | | Supported by cDNA | | | |
| 267 | Gma.6192.1.S1_s_at | 0.44 | AW396773 | Metabolism | | | |
| 268 | GmaAffx.60057.1.S1_at | 0.439 | BM308158 | No Homology to Known Proteins | | | |
| 269 | Gma.6151.1.S1_at | 0.438 | BQ611806 | Metabolism | | | Flavin-containing monooxygenase |
| 270 | GmaAffx.21041.1.A1_at | 0.438 | AW350819 | No Homology to Known Proteins | | | |
| 271 | Gma.13030.1.S1_a_at | 0.437 | AI748012 | Protein Destination & Storage | | | |
| 272 | GmaAffx.90600.1.S1_at | 0.437 | CF806325 | Metabolism | | | |
| 273 | GmaAffx.59808.1.S1_at | 0.435 | BM886228 | Transporter | | | Predicted transporter (major |
| | | | | | | | facilitator superfamily) |
| 274 | Gma.2776.1.S1_at | 0.434 | BI968513 | Secondary Metabolism | Ethylene | | Iron/ascorbate family oxidoreductases |
| | | | | | Biosynthesis | | |
| 275 | GmaAffx.15983.2.S1_at | 0.434 | AW279540 | Signal Transduction | Protein | | Serine/threonine protein phosphatase |
| | | | | | Phosphatase | | |
| 276 | Gma.7788.1.A1_at | 0.434 | BU549148 | Unclassified - Hypothetical Protein | | | Aspartyl protease |
| | | | | Supported by cDNA | | | |
| 277 | GmaAffx.11049.1.S1_at | 0.432 | BU545140 | Unclassified - Protein with | | | Phosphatidylethanolamine binding |
| | | | | Unknown Function | | | protein |
| 278 | Gma.2776.2.S1_s_at | 0.432 | BU577475 | Secondary Metabolism | Ethylene | | Iron/ascorbate family oxidoreductases |
| | | | | | Biosynthesis | | |
| 279 | GmaAffx.91807.1.S1_at | 0.432 | CF807532 | No Homology to Known Proteins | | | |
| 280 | GmaAffx.87417.1.S1_at | 0.43 | BM568072 | Metabolism | | | |
| 281 | GmaAffx.48042.1.S1_at | 0.429 | AI960210 | Transporter | | | Aquaporin (major intrinsic protein |
| | | | | | | | family) |
| 282 | GmaAffx.85999.1.S1_at | 0.429 | BF426048 | Unclassified - Hypothetical Protein | | | |
| | | | | Supported by cDNA | | | |
| 283 | Gma.3963.2.S1_at | 0.429 | BE608192 | Unclassified - Hypothetical Protein | | | Zn-finger protein |
| | | | | NOT Supported by cDNA | | | |
| 284 | GmaAffx.12014.1.A1_at | 0.428 | CA802431 | Unclassified - Hypothetical Protein | | | NADH pyrophosphatase 1 of the |
| | | | | Supported by cDNA | | | Nudix family of hydrolases |
| 285 | GmaAffx.59210.1.S1_at | 0.427 | BI967182 | Secondary Metabolism | Ethylene | | Iron/ascorbate family oxidoreductases |
| | | | | | Biosynthesis | | |
| 286 | Gma.1243.1.S1_at | 0.426 | AW311027 | Unclassified - Hypothetical Protein | | | |
| | | | | Supported by cDNA | | | |
| 287 | Gma.3450.1.S1_at | 0.425 | BE657895 | Unclassified - Hypothetical Protein | | | |
| | | | | Supported by cDNA | | | |
| 288 | Gma.5159.1.A1_at | 0.425 | BI971147 | No Homology to Known Proteins | | | |
| 289 | GmaAffx.15471.3.S1_x_at | 0.425 | BE346777 | Transcription | Transcription | MYB | Transcription factor, Myb |
| | | | | | Factor | | superfamily |
| 290 | GmaAffx.9280.1.S1_at | 0.425 | BI498912 | Signal Transduction | Calcium | Calcium | |
| | | | | | Binding | Dependent | |
| | | | | | | Protein | |
| | | | | | | Kinase | |
| 291 | Gma.1661.1.S1_at | 0.424 | BE821382 | Metabolism | | | |
| 292 | GmaAffx.91428.1.A1_at | 0.423 | CF807137 | Metabolism | | | |
| 293 | Gma.1061.1.A1_at | 0.421 | CD408183 | Metabolism | | | |
| 294 | GmaAffx.59014.1.S1_s_at | 0.421 | AW733977 | No Homology to Known Proteins | | | |
| 295 | Gma.10536.1.S1_at | 0.42 | BG508300 | No Homology to Known Proteins | | | |
| 296 | Gma.5478.1.S1_at | 0.419 | AW350606 | Metabolism | | | |
| 297 | GmaAffx.4036.1.S1_at | 0.419 | BM527313 | Unclassified - Hypothetical Protein | | | |
| | | | | Supported by cDNA | | | |
| 298 | Gma.15468.1.S1_at | 0.418 | BE800435 | Disease & Defense | | | |
| 299 | Gma.1495.1.S1_at | 0.418 | BI973545 | No Homology to Known Proteins | | | |
| 300 | GmaAffx.53099.1.S1_at | 0.418 | BG043340 | Unclassified - Hypothetical Protein | | | Alpha-L-fucosidase |
| | | | | NOT Supported by cDNA | | | |
| 301 | GmaAffx.85287.2.S1_at | 0.417 | BI472203 | Unclassified - Hypothetical Protein | | | |
| | | | | Supported by cDNA | | | |
| 302 | Gma.5586.2.S1_at | 0.416 | CD398384 | Transcription | Transcription | Unclassified | CCAAT-binding factor, subunit B |
| | | | | | Factor | | (HAP2) |
| 303 | GmaAffx.81502.1.S1_at | 0.415 | CD404946 | Unclassified - Hypothetical Protein | | | Uncharacterized conserved protein |
| | | | | NOT Supported by cDNA | | | |
| 304 | GmaAffx.83016.1.S1_at | 0.415 | BQ080317 | Transcription | Transcription | Zinc Finger | |
| | | | | | Factor | | |
| 305 | Gma.1445.1.S1_at | 0.414 | BQ454241 | Metabolism | | | Flavonol reductase/cinnamoyl-CoA |
| | | | | | | | reductase |
| 306 | Gma.3384.1.S1_at | 0.414 | BE659400 | Unclassified - Protein with | | | Acyl-CoA reductase |
| | | | | Unknown Function | | | |
| 307 | GmaAffx.948.1.S1_at | 0.414 | BG155389 | Protein Destination & Storage | | | Ubiquitin fusion-degradation protein |
| 308 | Gma.6684.1.S1_at | 0.413 | AW459859 | Signal Transduction | Unclassified | | Lanthionine synthetase C-like protein 1 |
| 309 | Gma.3963.1.S1_at | 0.413 | AW202007 | Unclassified - Hypothetical Protein | | | Zn-finger protein |
| | | | | NOT Supported by cDNA | | | |
| 310 | GmaAffx.8999.2.A1_s_at | 0.413 | BI700574 | No Homology to Known Proteins | | | |
| 311 | Gma.4687.1.S1_at | 0.412 | AW309716 | Secondary Metabolism | | | |
| 312 | Gma.6192.1.S1_at | 0.411 | AW396773 | Metabolism | | | |
| 313 | GmaAffx.81287.1.S1_at | 0.411 | BG789983 | Secondary Metabolism | | | Hydroxyindole-O-methyltransferase |
| | | | | | | | and related SAM-dependent |
| | | | | | | | methyltransferases |
| 314 | Gma.3591.2.S1_at | 0.409 | BE473658 | Secondary Metabolism | Ethylene | | Iron/ascorbate family oxidoreductases |
| | | | | | Biosynthesis | | |
| 315 | GmaAffx.29616.1.A1_at | 0.409 | CD404723 | No Homology to Known Proteins | | | |
| 316 | GmaAffx.74160.1.S1_at | 0.409 | BQ740845 | Protein Destination & Storage | | | |
| 317 | Gma.10969.3.S1_x_at | 0.407 | CD396552 | Metabolism | | | |
| 318 | Gma.16910.1.S1_at | 0.406 | BU549219 | Unclassified - Protein with | | | |
| | | | | Unknown Function | | | |
| 319 | Gma.1758.1.S1_at | 0.406 | CD402046 | Unclassified - Protein with | | | |
| | | | | Unknown Function | | | |
| 320 | PsAffx.C107000004_at | 0.402 | | | | | |
| 321 | Gma.3914.1.S1_at | 0.401 | BI702206 | Unclassified - Hypothetical Protein | | | |
| | | | | Supported by cDNA | | | |
| 322 | Gma.1394.1.S1_at | 0.4 | BG839300 | Metabolism | | | |
| 323 | Gma.8489.1.S1_s_at | 0.4 | AB030493.1 | Metabolism | | | Protein involved in thiamine |
| | | | | | | | biosynthesis and DNA damage |
| | | | | | | | tolerance |
| 324 | GmaAffx.42732.1.S1_at | 0.399 | BU765857 | Protein Destination & Storage | Ubiquitin | | FOG: Predicted E3 ubiquitin ligase |
| | | | | | Degradation | | |
| 325 | GmaAffx.93342.1.S1_s_at | 0.399 | CF809067 | Disease & Defense | | | Glutathione peroxidase |
| 326 | GmaAffx.61777.1.S1_at | 0.399 | AI965744 | Signal Transduction | Protein | | |
| | | | | | Kinase | | |
| 327 | GmaAffx.70005.1.S1_s_at | 0.395 | CA936335 | Secondary Metabolism | | | Iron/ascorbate family oxidoreductases |
| 328 | Gma.1951.2.S1_a_at | 0.392 | CA783485 | Protein Destination & Storage | | | Serine carboxypeptidases |
| 329 | GmaAffx.77203.1.S1_at | 0.39 | BI970687 | Unclassified - Hypothetical Protein | | | |
| | | | | Supported by cDNA | | | |
| 330 | Gma.1394.2.S1_at | 0.389 | BI470370 | Metabolism | | | |
| 331 | Gma.4252.1.S1_at | 0.389 | BE821208 | Metabolism | | | Glutamate decarboxylase and related |
| | | | | | | | proteins |
| 332 | GmaAffx.14153.2.S1_at | 0.387 | BE329834 | Unclassified - Hypothetical Protein | | | |
| | | | | Supported by cDNA | | | |
| 333 | Gma.3578.1.S1_at | 0.386 | AW156522 | Disease & Defense | | | Acyl-CoA synthetase |
| 334 | Gma.8946.1.S1_at | 0.386 | BQ453813 | Transcription | Transcription | NAC Domain | |
| | | | | | Factor | | |
| 335 | GmaAffx.34449.1.S1_at | 0.386 | AW164447 | No Homology to Known Proteins | | | |
| 336 | Gma.3420.1.S1_at | 0.385 | CD406082 | Unclassified - Hypothetical Protein | | | |
| | | | | Supported by cDNA | | | |
| 337 | Gma.4150.2.S1_s_at | 0.384 | AW350622 | Unclassified - Hypothetical Protein | | | |
| | | | | Supported by cDNA | | | |
| 338 | GmaAffx.15196.1.S1_at | 0.384 | BU549436 | Metabolism | | | Long-chain acyl-CoA synthetases |
| | | | | | | | (AMP-forming) |
| 339 | Gma.16359.LA1_at | 0.384 | BI970359 | Metabolism | | | GPI-alpha-mannosyltransferase III |
| | | | | | | | (GPI10/PIG-B) involved in |
| | | | | | | | glycosylphosphatidylinositol anchor |
| | | | | | | | biosynthesis |
| 340 | GmaAffx.29951.2.S1_at | 0.383 | AW100899 | Metabolism | | | Dehydrogenase kinase |
| 341 | Gma.6033.1.S1_at | 0.382 | BI967767 | Unclassified - Hypothetical Protein | | | |
| | | | | Supported by cDNA | | | |
| 342 | Gma.3987.2.S1_a_at | 0.381 | BI971594 | No Homology to Known Proteins | | | |
| 343 | GmaAffx.12142.1.S1_at | 0.38 | AW733336 | Metabolism | Cytochrome | CYP716A | Cytochrome P450 |
| | | | | | P450 | | CYP4/CYP19/CYP26 subfamilies |
| 344 | GmaAffx.57432.2.S1_at | 0.38 | CA819434 | No Homology to Known Proteins | | | |
| 345 | GmaAffx.90600.1.S1_s_at | 0.379 | CF806325 | Metabolism | | | |
| 346 | GmaAffx.93212.1.S1_s_at | 0.379 | CF807524 | Transporter | | | |
| 347 | GmaAffx.61311.1.S1_at | 0.378 | CA936951 | Transporter | | | Amino acid transporters |
| 348 | Gma.2540.2.S1_s_at | 0.378 | BI787366 | No Homology to Known Proteins | | | |
| 349 | Gma.4099.1.S1_at | 0.378 | BG651846 | Metabolism | | | Beta-galactosidase |
| 350 | Gma.8262.1.A1_at | 0.377 | BU550168 | Signal Transduction | Protein | | Putative serine/threonine protein |
| | | | | | Kinase | | kinase |
| 351 | Gma.17402.1.A1_at | 0.376 | BE611084 | No Homology to Known Proteins | | | |
| 352 | Gma.4593.1.S1_a_at | 0.373 | CD396298 | Energy | | | |
| 353 | Gma.2388.1.S1_at | 0.372 | CD416539 | No Homology to Known Proteins | | | |
| 354 | Gma.13418.3.S1_at | 0.372 | CA938391 | Transporter | | | |
| 355 | Gma.1326.1.S1_at | 0.37 | CD397515 | Secondary Metabolism | | | |
| 356 | Gma.7227.1.S1_at | 0.37 | BU551112 | Protein Destination & Storage | | | Ubiquitin-protein ligase |
| 357 | GmaAffx.78358.2.S1_at | 0.37 | BU762686 | Metabolism | | | |
| 358 | Gma.1490.1.S1_at | 0.369 | AB000097.1 | Cell Structure | | | Chitinase |
| 359 | GmaAffx.58125.1.A1_at | 0.368 | BE347561 | Transcription | Transcription | Basic Helix- | |
| | | | | | Factor | Loop-Helix | |
| | | | | | | (bHLH) | |
| 360 | GmaAffx.36470.1.A1_at | 0.367 | BU550552 | Unclassified - Hypothetical Protein | | | |
| | | | | Supported by cDNA | | | |
| 361 | Gma.7831.1.S1_s_at | 0.367 | CD393602 | Transporter | | | |
| 362 | GmaAffx.87900.1.S1_at | 0.367 | BI892943 | Protein Destination & Storage | | | FKBP-type peptidyl-prolyl cis-trans |
| | | | | | | | isomerase |
| 363 | Gma.5513.1.S1_at | 0.367 | AW310453 | Metabolism | | | Haloacid dehalogenase-like hydrolase |
| 364 | Gma.1164.1.S1_at | 0.366 | BE822845 | Energy | | | Ferric reductase, NADH/NADPH |
| | | | | | | | oxidase and related proteins |
| 365 | Gma.17323.1.S1_at | 0.366 | AW567763 | No Homology to Known Proteins | | | |
| 366 | GmaAffx.91731.1.S1_s_at | 0.363 | CF807456 | Secondary Metabolism | Ethylene | | Iron/ascorbate family oxidoreductases |
| | | | | | Biosynthesis | | |
| 367 | Gma.2066.1.S1_a_at | 0.362 | BG363704 | No Homology to Known Proteins | | | |
| 368 | Gma.11166.1.S1_x_at | 0.359 | AW705829 | Metabolism | | | |
| 369 | GmaAffx.71308.1.S1_at | 0.358 | BF595112 | Transcription | Transcription | Heat Shock | Heat shock transcription factor |
| | | | | | Factor | | |
| 370 | GmaAffx.82936.1.S1_at | 0.358 | AI938364 | Unclassified - Protein with | | | |
| | | | | Unknown Function | | | |
| 371 | GmaAffx.93328.1.S1_s_at | 0.357 | CF809053 | Secondary Metabolism | | | |
| 372 | GmaAffx.93591.1.S1_s_at | 0.354 | CF809316 | Metabolism | | | |
| 373 | GmaAffx.64807.1.S1_at | 0.354 | AW201972 | No Homology to Known Proteins | | | |
| 374 | Gma.857.1.S1_at | 0.352 | BE822264 | No Homology to Known Proteins | | | |
| 375 | Gma.7580.1.S1_at | 0.351 | BE057763 | Unclassified - Hypothetical Protein | | | |
| | | | | Supported by cDNA | | | |
| 376 | Gma.3591.1.S1_a_at | 0.35 | BE440266 | Secondary Metabolism | Ethylene | | Iron/ascorbate family oxidoreductases |
| | | | | | Biosynthesis | | |
| 377 | Gma.10216.3.A1_x_at | 0.348 | BI969715 | Metabolism | | | Hydroxyindole-O-methyltransferase |
| | | | | | | | and related SAM-dependent |
| | | | | | | | methyltransferases |
| 378 | Gma.5162.1.A1_at | 0.347 | BI971156 | Signal Transduction | Protein | | |
| | | | | | Kinase | | |
| 379 | GmaAffx.17892.2.S1_at | 0.345 | BQ628677 | No Homology to Known Proteins | | | |
| 380 | Gma.11166.1.S1_s_at | 0.344 | AW705829 | Metabolism | | | |
| 381 | GmaAffx.54778.1.S1_at | 0.344 | BI322039 | No Homology to Known Proteins | | | |
| 382 | Gma.9647.1.S1_at | 0.343 | BE820997 | Cell Structure | | | O-methyltransferase |
| 383 | GmaAffx.11242.1.S1_s_at | 0.343 | AW317461 | Protein Destination & Storage | | | Serine carboxypeptidases |
| 384 | GmaAffx.80584.1.S1_at | 0.343 | BU083387 | Transcription | Transcription | Basic Helix- | |
| | | | | | Factor | Loop-Helix | |
| | | | | | | (bHLH) | |
| 385 | Gma.9740.1.S1_at | 0.342 | BQ081058 | Unclassified - Hypothetical Protein | | | |
| | | | | Supported by cDNA | | | |
| 386 | Gma.5760.2.S1_at | 0.341 | BQ298381 | Disease & Defense | | | |
| 387 | Gma.5929.1.S1_at | 0.341 | BQ610446 | No Homology to Known Proteins | | | |
| 388 | Gma.13150.1.A1_at | 0.341 | CD411577 | Unclassified - Protein with | Hormone | | |
| | | | | Unknown Function | Induced | | |
| 389 | GmaAffx.53324.1.A1_at | 0.341 | AW309251 | Transcription | Transcription | Basic Helix- | |
| | | | | | Factor | Loop-Helix | |
| | | | | | | (bHLH) | |
| 390 | Gma.4643.1.S1_at | 0.339 | BU547906 | Transcription | Transcription | NAC Domain | |
| | | | | | Factor | | |
| 391 | Gma.398.2.S1_at | 0.338 | BU925900 | Metabolism | | | Homocysteine S-methyltransferase |
| 392 | GmaAffx.92791.1.S1_at | 0.336 | CF808516 | Metabolism | | | Reductases with broad range of |
| | | | | | | | substrate specificities |
| 393 | Gma.17516.1.S1_at | 0.336 | CD396417 | Transporter | | | Ca2+/H+ antiporter VCX1 and |
| | | | | | | | related proteins |
| 394 | GmaAffx.60479.2.S1_at | 0.334 | BU765815 | Transcription | Transcription | Homeodomain | Transcription factor ME1S1 and |
| | | | | | Factor | | related HOX domain proteins |
| 395 | GmaAffx.68507.1.S1_at | 0.333 | BI699405 | Transcription | Transcription | Heat Shock | Heat shock transcription factor |
| | | | | | Factor | | |
| 396 | Gma.16500.1.S1_at | 0.332 | BE059054 | Metabolism | | | |
| 397 | GmaAffx.32011.1.S1_at | 0.332 | BI701198 | No Homology to Known Proteins | | | |
| 398 | GmaAffx.83715.1.S1_at | 0.331 | BI785710 | Transporter | | | Glutaredoxin-related protein |
| 399 | GmaAffx.11897.1.S1_at | 0.33 | BE819868 | Transporter | | | Permease of the major facilitator |
| | | | | | | | superfamily |
| 400 | Gma.4728.1.A1_at | 0.328 | BI968944 | Secondary Metabolism | | | NADH: flavin oxidoreductase/12- |
| | | | | | | | oxophytodienoate reductase |
| 401 | Gma.15307.1.S1_at | 0.326 | U64866.1 | Disease & Defense | | | |
| 402 | GmaAffx.79516.1.A1_at | 0.326 | CD400596 | Transcription | Transcription | MYB | |
| | | | | | Factor | | |
| 403 | GmaAffx.64967.1.S1_at | 0.325 | BU546115 | Secondary Metabolism | | | Iron/ascorbate family oxidoreductases |
| 404 | Gma.1951.1.S1_at | 0.321 | BQ079657 | Protein Destination & Storage | | | Serine carboxypeptidases |
| 405 | GmaAffx.63464.1.S1_at | 0.318 | BG156739 | No Homology to Known Proteins | | | |
| 406 | Gma.10591.2.S1_at | 0.318 | CD414199 | Metabolism | | | Glycine cleavage system H protein |
| | | | | | | | (lipoate-binding) |
| 407 | GmaAffx.61130.1.S1_at | 0.314 | CA799668 | Transporter | | | H+/oligopeptide symporter |
| 408 | Gma.1314.1.S1_at | 0.314 | U63726.1 | Metabolism | | | Gamma-glutamyl hydrolase |
| 409 | GmaAffx.88546.1.S1_at | 0.313 | BE801134 | Metabolism | | | |
| 410 | GmaAffx.64794.1.S1_at | 0.313 | CA801749 | Unclassified - Hypothetical Protein | | | |
| | | | | Supported by cDNA | | | |
| 411 | Gma.1394.2.S1_a_at | 0.313 | BI470370 | Metabolism | | | |
| 412 | Gma.12227.1.S1_at | 0.312 | BG511671 | Unclassified - Hypothetical Protein | | | |
| | | | | NOT Supported by cDNA | | | |
| 413 | GmaAffx.32011.2.S1_at | 0.312 | BM084966 | Transcription | Transcription | Basic | |
| | | | | | Factor | Leucine | |
| | | | | | | Zipper | |
| | | | | | | (bZIP) | |
| 414 | Gma.177.1.S1_at | 0.312 | AF004806.1 | Protein Destination & Storage | Storage | | Reticulon |
| | | | | | Protein | | |
| 415 | Gma.8022.1.S1_at | 0.311 | BQ628998 | Disease & Defense | | | Soluble epoxide hydrolase |
| 416 | GmaAffx.27331.1.S1_at | 0.31 | BM520297 | Unclassified - Hypothetical Protein | | | |
| | | | | NOT Supported by cDNA | | | |
| 417 | GmaAffx.33677.1.S1_at | 0.308 | CA782582 | No Homology to Known Proteins | | | |
| 418 | GmaAffx.66215.1.S1_at | 0.308 | AI443109 | Signal Transduction | Unclassified | | Lanthionine synthetase C-like protein 1 |
| 419 | GmaAffx.77832.1.S1_at | 0.305 | BI971626 | Unclassified - Hypothetical Protein | | | |
| | | | | NOT Supported by cDNA | | | |
| 420 | PsAffx.psMA015xP13f_at | 0.305 | | | | | |
| 421 | GmaAffx.17961.1.A1_at | 0.303 | BE657601 | Protein Destination & Storage | | | Molecular chaperones |
| | | | | | | | HSP70/HSC70, HSP70 superfamily |
| 422 | Gma.728.1.A1_at | 0.3 | BE658779 | No Homology to Known Proteins | | | |
| 423 | GmaAffx.31640.1.S1_at | 0.3 | BG237979 | No Homology to Known Proteins | | | |
| 424 | Gma.4874.1.A1_at | 0.298 | AI938293 | No Homology to Known Proteins | | | |
| 425 | Gma.11028.2.S1_s_at | 0.297 | CA782711 | Metabolism | | | Glyoxylate/hydroxypyruvate |
| | | | | | | | reductase (D-isomer-specific 2- |
| | | | | | | | hydroxy acid dehydrogenase |
| | | | | | | | superfamily) |
| 426 | GmaAffx.50980.1.S1_s_at | 0.296 | BE823095 | Signal Transduction | Protein | | |
| | | | | | Phosphatase | | |
| 427 | GmaAffx.26767.1.S1_at | 0.295 | BF010276 | Transcription | Transcription | MYB | Transcription factor, Myb |
| | | | | | Factor | | superfamily |
| 428 | Gma.5513.1.S1_x_at | 0.295 | AW310453 | Metabolism | | | Haloacid dehalogenase-like hydrolase |
| 429 | GmaAffx.78163.1.S1_s_at | 0.293 | BM520857 | Signal Transduction | Protein | | Serine/threonine protein phosphatase |
| | | | | | Phosphatase | | |
| 430 | GmaAffx.11172.1.A1_at | 0.293 | CD404556 | No Homology to Known Proteins | | | |
| 431 | Gma.8530.1.S1_at | 0.292 | AF109720.1 | Protein Destination & Storage | Storage | | |
| | | | | | Protein | | |
| 432 | GmaAffx.5255.1.S1_at | 0.292 | BG362929 | Transcription | Transcription | AP2/EREBP | |
| | | | | | Factor | | |
| 433 | Gma.3118.1.S1_s_at | 0.29 | BQ786048 | Disease & Defense | | | Predicted metallothionein |
| 434 | Gma.8058.1.A1_at | 0.289 | BM271479 | Signal Transduction | Protein | | Serine/threonine protein phosphatase |
| | | | | | Phosphatase | | |
| 435 | Gma.7437.1.S1_at | 0.289 | BU547705 | Unclassified - Hypothetical Protein | | | |
| | | | | Supported by cDNA | | | |
| 436 | GmaAffx.85382.1.S1_s_at | 0.289 | BI945154 | No Homology to Known Proteins | | | |
| 437 | Gma.3744.2.S1_a_at | 0.288 | BI892693 | Unclassified - Protein With | | | |
| | | | | Unknown Function | | | |
| 438 | GmaAffx.56319.1.S1_at | 0.288 | BE802287 | Metabolism | | | Uncharacterized conserved protein |
| 439 | Gma.13452.1.A1_at | 0.287 | CD398445 | No Homology to Known Proteins | | | |
| 440 | GmaAffx.58712.1.S1_at | 0.287 | BF325034 | Transporter | | | Transporter, ABC superfamily |
| | | | | | | | (Breast cancer resistance protein) |
| 441 | GmaAffx.3369.2.A1_at | 0.286 | BU550316 | Cell Structure | | | |
| 442 | Gma.17688.1.S1_s_at | 0.285 | CA937360 | Transcription | Transcription | Basic Helix- | |
| | | | | | Factor | Loop-Helix | |
| | | | | | | (bHLH) | |
| 443 | Gma.5469.1.S1_at | 0.284 | AW349200 | No Homology to Known Proteins | | | |
| 444 | GmaAffx.89624.1.A1_s_at | 0.279 | CK606100 | Transcription | Transcription | Basic | |
| | | | | | Factor | Leucine | |
| | | | | | | Zipper | |
| | | | | | | (bZlP) | |
| 445 | Gma.649.1.S1_at | 0.274 | AW568984 | No Homology to Known Proteins | | | |
| 446 | GmaAffx.90395.1.A1_s_at | 0.274 | CF807499 | Unclassified - Protein with | | | |
| | | | | Unknown Function | | | |
| 447 | Gma.595.1.S1_at | 0.273 | AB004271.1 | Metabolism | | | |
| 448 | GmaAffx.59252.1.A1_at | 0.272 | AI442669 | Protein Destination & Storage | | | FOG: Predicted E3 ubiquitin ligase |
| 449 | Gma.3744.1.S1_at | 0.27 | CD417438 | Unclassified - Protein With | | | |
| | | | | Unknown Function | | | |
| 450 | GmaAffx.70435.2.S1_at | 0.266 | AW705853 | Transcription | Transcription | Basic Helix- | |
| | | | | | Factor | Loop-Helix | |
| | | | | | | (bHLH) | |
| 451 | Gma.14471.1.S1_s_at | 0.265 | CF921241 | No Homology to Known Proteins | | | |
| 452 | Gma.2253.1.S1_at | 0.264 | AF169018.1 | Metabolism | | | Reductases with broad range of |
| | | | | | | | substrate specificities |
| 453 | Gma.12731.2.A1_s_at | 0.263 | CD395848 | Unclassified - Hypothetical Protein | | | Aquaporin (major intrinsic protein |
| | | | | NOT Supported by cDNA | | | family) |
| 454 | GmaAffx.34657.1.A1_s_at | 0.261 | AW351012 | No Homology to Known Proteins | | | |
| 455 | Gma.2086.1.S1_at | 0.258 | AF035671.1 | Metabolism | Amino Acid | | Arginase family protein |
| | | | | | Biosynthesis/ | | |
| | | | | | Metabolism | | |
| 456 | GmaAffx.34975.1.S1_at | 0.256 | BU547817 | Unclassified - Hypothetical Protein | | | |
| | | | | Supported by cDNA | | | |
| 457 | Gma.10227.1.A1_at | 0.253 | BE020065 | Metabolism | | | Flavin-containing monooxygenase |
| 458 | Gma.10896.3.A1_at | 0.253 | BI316837 | Metabolism | | | Aldehyde dehydrogenase |
| 459 | Gma.968.1.S1_at | 0.253 | D28876.1 | Protein Destination & Storage | | | Asparaginyl peptidases |
| 460 | GmaAffx.26908.1.S1_at | 0.253 | BF425834 | Secondary Metabolism | | | Beta, beta-carotene 15,15′- |
| | | | | | | | dioxygenase and related enzymes |
| 461 | GmaAffx.25612.1.S1_at | 0.252 | AW317577 | No Homology to Known Proteins | | | |
| 462 | GmaAffx.84424.1.S1_at | 0.252 | AW311543 | Metabolism | | | Alcohol dehydrogenase, class V |
| 463 | GmaAffx.17918.1.S1_at | 0.25 | BG157024 | No Homology to Known Proteins | | | |
| 464 | Gma.10904.1.S1_s_at | 0.247 | CD408184 | Protein Destination & Storage | | | |
| 465 | GmaAffx.2710.1.S1_at | 0.247 | BE822538 | Unclassified - Hypothetical Protein | | | Endoplasmic reticulum protein EP58, |
| | | | | Supported by cDNA | | | contains filamin rod domain and |
| | | | | | | | KDEL motif |
| 466 | GmaAffx.45957.2.S1_at | 0.247 | BI974135 | No Homology to Known Proteins | | | |
| 467 | GmaAffx.11815.1.S1_at | 0.246 | AW311637 | Metabolism | | | |
| 468 | Gma.11731.1.A1_at | 0.243 | CD392320 | Unclassified - Protein with | | | |
| | | | | Unknown Function | | | |
| 469 | GmaAffx.32606.1.S1_at | 0.241 | BM270707 | Unclassified - Hypothetical Protein | | | Aquaporin (major intrinsic protein |
| | | | | NOT Supported by cDNA | | | family) |
| 470 | GmaAffx.80188.1.A1_at | 0.239 | AW349832 | No Homology to Known Proteins | | | |
| 471 | GmaAffx.57970.1.S1_at | 0.238 | BM886862 | Transcription | Transcription | NAC Domain | |
| | | | | | Factor | | |
| 472 | GmaAffx.40923.1.S1_at | 0.238 | BG653848 | Disease & Defense | | | Selenium-binding protein |
| 473 | GmaAffx.52826.3.S1_at | 0.237 | BF596503 | Signal Transduction | Protein | | Serine/threonine protein phosphatase |
| | | | | | Phosphatase | | |
| 474 | GmaAffx.10582.1.S1_at | 0.234 | BM523687 | Transcription | Transcription | CCAAT | CCAAT-binding factor, subunit A |
| | | | | | Factor | | (HAP3) |
| 475 | GmaAffx.82263.1.S1_s_at | 0.234 | AW568454 | No Homology to Known Proteins | | | |
| 476 | Gma.12068.1.A1_at | 0.232 | BU547739 | Transporter | | | Transporter, ABC superfamily |
| | | | | | | | (Breast cancer resistance protein) |
| 477 | GmaAffx.71308.2.A1_at | 0.23 | AW569888 | No Homology to Known Proteins | | | |
| 478 | GmaAffx.63054.1.S1_at | 0.229 | BE806784 | Unclassified - Hypothetical Protein | | | |
| | | | | NOT Supported by cDNA | | | |
| 479 | Gma.12684.1.S1_at | 0.226 | CD394773 | Transcription | Transcription | Zinc Finger | |
| | | | | | Factor | | |
| 480 | GmaAffx.19118.1.S1_at | 0.225 | BM523731 | Unclassified - Hypothetical Protein | | | |
| | | | | NOT Supported by cDNA | | | |
| 481 | Gma.1527.1.S1_x_at | 0.22 | BI968185 | Secondary Metabolism | | | Flavonol reductase/cinnamoyl-CoA |
| | | | | | | | reductase |
| 482 | Gma.5331.1.S1_at | 0.22 | BI892702 | Transcription | Transcription | NAC Domain | |
| | | | | | Factor | | |
| 483 | GmaAffx.30792.1.A1_at | 0.22 | BU544815 | Unclassified - Hypothetical Protein | | | |
| | | | | Supported by cDNA | | | |
| 484 | Gma.17688.1.S1_at | 0.219 | CA937360 | Transcription | Transcription | Basic Helix- | |
| | | | | | Factor | Loop-Helix | |
| | | | | | | (bHLH) | |
| 485 | Gma.7824.1.S1_at | 0.219 | BU550492 | Metabolism | | | Kynurenine 3-monooxygenase and |
| | | | | | | | related flavoprotein monooxygenases |
| 486 | Gma.1768.1.S1_s_at | 0.219 | BE820777 | Unclassified - Hypothetical Protein | | | |
| | | | | Supported by cDNA | | | |
| 487 | GmaAffx.81362.1.S1_at | 0.218 | BF324988 | Cell Structure | | | |
| 488 | GmaAffx.38555.2.S1_at | 0.214 | BI425873 | No Homology to Known Proteins | | | |
| 489 | Gma.11873.1.S1_at | 0.207 | CA819977 | Unclassified - Protein with | | | |
| | | | | Unknown Function | | | |
| 490 | Gma.2887.2.S1_x_at | 0.206 | BI701520 | Secondary Metabolism | | | Phenylalanine and histidine ammonia- |
| | | | | | | | lyase |
| 491 | Gma.17985.2.S1_at | 0.205 | BE802263 | Metabolism | | | |
| 492 | GmaAffx.90113.1.S1_at | 0.202 | CF806004 | Protein Destination & Storage | Storage | | Uncharacterized conserved protein |
| | | | | | Protein | | |
| 493 | GmaAffx.26868.1.S1_at | 0.201 | BF325443 | Unclassified - Hypothetical Protein | | | Tomosyn and related SNARE- |
| | | | | NOT Supported by cDNA | | | interacting proteins |
| 494 | GmaAffx.91338.1.S1_at | 0.2 | CF807063 | Unclassified - Hypothetical Protein | | | Predicted haloacid dehalogenase-like |
| | | | | Supported by cDNA | | | hydrolase |
| 495 | GmaAffx.50980.1.S1_x_at | 0.199 | BE823095 | Signal Transduction | Protein | | |
| | | | | | Phosphatase | | |
| 496 | Gma.13854.1.S1_at | 0.199 | CD408166 | Metabolism | | | |
| 497 | GmaAffx.50980.1.S1_at | 0.197 | BE823095 | Signal Transduction | Protein | | |
| | | | | | Phosphatase | | |
| 498 | GmaAffx.31862.1.S1_at | 0.192 | BE822501 | Unclassified - Hypothetical Protein | | | |
| | | | | Supported by cDNA | | | |
| 499 | GmaAffx.93212.1.S1_at | 0.191 | CF807524 | Transporter | | | |
| 500 | GmaAffx.90395.1.S1_s_at | 0.186 | CF806120 | Unclassified - Protein with | | | |
| | | | | Unknown Function | | | |
| 501 | Gma.15.1.S1_at | 0.185 | U66316.1 | Unclassified - Protein with | | | |
| | | | | Unknown Function | | | |
| 502 | GmaAffx.9911.1.S1_at | 0.184 | BU547808 | Unclassified - Hypothetical Protein | | | Tomosyn and related SNARE- |
| | | | | NOT Supported by cDNA | | | interacting proteins |
| 503 | GmaAffx.81851.1.S1_at | 0.181 | AW706751 | Unclassified - Hypothetical Protein | | | |
| | | | | Supported by cDNA | | | |
| 504 | GmaAffx.12665.1.A1_at | 0.176 | BE822073 | Metabolism | | | |
| 505 | GmaAffx.89772.8.S1_at | 0.17 | CK605778 | Metabolism | | | |
| 506 | GmaAffx.91479.1.S1_s_at | 0.17 | CF807204 | No Homology to Known Proteins | | | |
| 507 | GmaAffx.89772.8.S1_x_at | 0.168 | CK605778 | Metabolism | | | |
| 508 | Gma.7227.2.S1_a_at | 0.163 | AW759183 | Protein Destination & Storage | | | Ubiquitin-protein ligase |
| 509 | Gma.16477.1.S1_at | 0.159 | BE824134 | No Homology to Known Proteins | | | |
| 510 | Gma.13062.1.S1_at | 0.157 | CD394315 | Metabolism | | | |
| 511 | Gma.12094.1.A1_at | 0.157 | CD395088 | Unclassified - Hypothetical Protein | | | |
| | | | | Supported by cDNA | | | |
| 512 | Gma.15093.1.S1_at | 0.153 | CD400374 | Protein Destination & Storage | | | Glutathione S-transferase |
| 513 | Gma.8187.1.S1_a_at | 0.152 | BU549133 | Signal Transduction | Protein | | Serine/threonine protein phosphatase |
| | | | | | Phosphatase | | |
| 514 | Gma.4837.1.S1_at | 0.147 | BI969343 | Transporter | | | H+/oligopeptide symporter |
| 515 | Gma.13296.3.S1_at | 0.147 | CD418313 | Transporter | | | |
| 516 | Gma.10904.1.S1_at | 0.141 | CD408184 | Protein Destination & Storage | | | |
| 517 | Gma.15534.1.S1_at | 0.133 | U50081.1 | Metabolism | | | |
| 518 | GmaAffx.93212.1.S1_x_at | 0.129 | CF807524 | Transporter | | | |
| 519 | GmaAffx.71222.1.S1_at | 0.124 | AW460050 | Metabolism | | | 5′-AMP-activated protein kinase, |
| | | | | | | | gamma subunit |
| 520 | Gma.10830.1.S1_at | 0.123 | BE820822 | Unclassified - Hypothetical Protein | | | |
| | | | | Supported by cDNA | | | |
| 521 | Gma.12393.3.S1_at | 0.121 | BM085488 | Secondary Metabolism | | | Beta, beta-carotene 15,15′- |
| | | | | | | | dioxygenase and related enzymes |
| 522 | GmaAffx.295.1.A1_s_at | 0.118 | BG653267 | Protein Destination & Storage | | | |
| 523 | GmaAffx.75697.1.S1_s_at | 0.113 | BU090518 | Unclassified - Hypothetical Protein | | | |
| | | | | Supported by cDNA | | | |
| 524 | GmaAffx.1785.1.S1_at | 0.112 | BF325331 | Transcription | Transcription | Homeodomain | Transcription factor HEX, contains |
| | | | | | Factor | | HOX and HALZ domains |
| 525 | Gma.1527.2.S1_at | 0.104 | BE658017 | Secondary Metabolism | | | Flavonol reductase/cinnamoyl-CoA |
| | | | | | | | reductase |
| 526 | GmaAffx.24413.1.A1_at | 0.103 | BU548368 | Metabolism | | | 5′-AMP-activated protein kinase, |
| | | | | | | | gamma subunit |
| 527 | Gma.1527.1.S1_a_at | 0.102 | BI968185 | Secondary Metabolism | | | Flavonol reductase/cinnamoyl-CoA |
| | | | | | | | reductase |
| 528 | Gma.176.1.S1_s_at | 0.101 | AF004810.1 | Protein Destination & Storage | Storage | | |
| | | | | | Protein | | |
| 529 | GmaAffx.90812.1.S1_at | 0.1 | CF806537 | Disease & Defense | | | |
| 530 | Gma.17985.1.S1_at | 0.099 | CD416450 | Metabolism | | | |
| 531 | Gma.6921.1.S1_at | 0.0986 | BE823209 | Signal Transduction | Unclassified | | Phosphatidylethanolamine binding |
| | | | | | | | protein |
| 532 | Gma.3774.2.S1_at | 0.0892 | CD393365 | Energy | | | Thioredoxin, nucleoredoxin and |
| | | | | | | | related proteins |
| 533 | GmaAffx.73698.1.S1_at | 0.0875 | BU964780 | Metabolism | | | 5′-AMP-activated protein kinase, |
| | | | | | | | gamma subunit |
| 534 | Gma.2986.1.S1_at | 0.0773 | BM271324 | Metabolism | | | 5′-AMP-activated protein kinase, |
| | | | | | | | gamma subunit |
| 535 | GmaAffx.12933.1.S1_at | 0.0755 | AW395787 | Transcription | Transcription | CCAAT | CCAAT-binding factor, subunit B |
| | | | | | Factor | | (HAP2) |
| 536 | Gma.3000.1.S1_at | 0.0728 | BE657172 | No Homology to Known Proteins | | | |
| 537 | Gma.17326.1.A1_at | 0.0703 | AW568254 | Unclassified - Hypothetical Protein | | | Permease of the major facilitator |
| | | | | NOT Supported by cDNA | | | superfamily |
| 538 | Gma.16566.1.S1_at | 0.0682 | CD400974 | Metabolism | | | Fumarase |
| 539 | Gma.3000.2.S1_a_at | 0.0673 | BM270844 | No Homology to Known Proteins | | | |
| 540 | Gma.9947.1.S1_at | 0.0631 | BF324269 | Unclassified - Hypothetical Protein | | | |
| | | | | Supported by cDNA | | | |
| 541 | GmaAffx.89407.1.A1_s_at | 0.0598 | CK606273 | Transporter | | | |
| 542 | Gma.8318.1.A1_at | 0.0496 | BU550469 | No Homology to Known Proteins | | | |
| 543 | Gma.3774.1.S1_at | 0.0478 | BU550698 | Energy | | | Thioredoxin, nucleoredoxin and |
| | | | | | | | related proteins |
| 544 | GmaAffx.10204.1.S1_at | 0.0455 | BQ298213 | Unclassified - Hypothetical Protein | | | |
| | | | | Supported by cDNA | | | |
| 545 | Gma.3774.2.S1_a_at | 0.0366 | CD393365 | Energy | | | Thioredoxin, nucleoredoxin and |
| | | | | | | | related proteins |
| 546 | GmaAffx.68621.1.A1_at | 0.0276 | BI893889 | Unclassified - Protein with | Stress | | |
| | | | | Unknown Function | Induced | | |
| 547 | Gma.2912.1.S1_at | 0.0224 | AF117884.1 | Protein Destination & Storage | Storage | | Uncharacterized conserved protein |
| | | | | | Protein | | |
| 548 | Gma.2912.1.S1_s_at | 0.0214 | AF117884.1 | Protein Destination & Storage | Storage | | Uncharacterized conserved protein |
| | | | | | Protein | | |

TABLE 8

| | Fold | Soybean_Gen | |
|---|---|---|---|
| ID | Change | Bank_Acc | Functional Category |
| 1 | 12.74 | BG651692 | Unclassified - Hypothetical Protein Supported by |
| | | | cDNA |
| 2 | 10.01 | CK606454 | No Homology to Known Proteins |
| 3 | 9.745 | CF808985 | Signal Transduction |
| 4 | 6.928 | Y10492.1 | Metabolism |
| 5 | 6.247 | BQ740550 | Disease & Defense |
| 6 | 5.327 | AW567677 | Unclassified - Hypothetical Protein Supported by |
| | | | cDNA |
| 7 | 4.791 | BE475550 | Cell Structure |
| 8 | 4.65 | AW350363 | Protein Destination & Storage |
| 9 | 4.508 | CA938421 | No Homology to Known Proteins |
| 10 | 4.501 | AJ518837.1 | Metabolism |
| 11 | 4.465 | CF808798 | Cell Structure |
| 12 | 4.336 | BI787790 | Unclassified - Hypothetical Protein Supported by |
| | | | cDNA |
| 13 | 4.332 | AW102083 | Metabolism |
| 14 | 4.291 | BU550417 | Unclassified - Hypothetical Protein NOT Supported by |
| | | | cDNA |
| 15 | 4.208 | CF805889 | Protein Destination & Storage |
| 16 | 4.126 | AW508123 | Unclassified - Protein with Unknown Function |
| 17 | 4.078 | AW099501 | Unclassified - Hypothetical Protein NOT Supported by |
| | | | cDNA |
| 18 | 4.027 | U64916.1 | Unclassified - Hypothetical Protein NOT Supported by |
| | | | cDNA |
| 19 | 3.791 | CF809254 | No Homology to Known Proteins |
| 20 | 3.77 | CA938421 | No Homology to Known Proteins |
| 21 | 3.768 | BU549896 | Unclassified - Hypothetical Protein NOT Supported by |
| | | | cDNA |
| 22 | 3.656 | AW100693 | No Homology to Known Proteins |
| 23 | 3.606 | AW350922 | Metabolism |
| 24 | 3.568 | BM954521 | Unclassified - Hypothetical Protein Supported by |
| | | | cDNA |
| 25 | 3.516 | BG510739 | Signal Transduction |
| 26 | 3.499 | BQ081355 | Metabolism |
| 27 | 3.41 | CF807071 | Unclassified - Hypothetical Protein Supported by |
| | | | cDNA |
| 28 | 3.402 | AW351023 | Metabolism |
| 29 | 3.401 | CF808262 | Energy |
| 30 | 3.337 | BF598665 | Unclassified - Hypothetical Protein NOT Supported by |
| | | | cDNA |
| 31 | 3.31 | BG508388 | No Homology to Known Proteins |
| 32 | 3.31 | BI971676 | Signal Transduction |
| 33 | 3.286 | BM954581 | Unclassified - Hypothetical Protein Supported by |
| | | | cDNA |
| 34 | 3.285 | CD398305 | No Homology to Known Proteins |
| 35 | 3.272 | CF809187 | Signal Transduction |
| 36 | 3.243 | BU544287 | No Homology to Known Proteins |
| 37 | 3.194 | BG789998 | Unclassified - Hypothetical Protein Supported by |
| | | | cDNA |
| 38 | 3.179 | BQ252812 | Transcription |
| 39 | 3.154 | BU544575 | No Homology to Known Proteins |
| 40 | 3.1 | BG507880 | Signal Transduction |
| 41 | 3.085 | CA818952 | Unclassified - Protein with Unknown Function |
| 42 | 3.068 | BU546096 | Signal Transduction |
| 43 | 3.037 | BM568170 | Unclassified - Hypothetical Protein Supported by |
| | | | cDNA |
| 44 | 3.014 | BU544524 | Signal Transduction |
| 45 | 2.939 | BE820254 | No Homology to Known Proteins |
| 46 | 2.932 | BE058913 | Metabolism |
| 47 | 2.913 | BM568087 | Signal Transduction |
| 48 | 2.884 | L12258.1 | Transporter |
| 49 | 2.85 | BG509282 | Disease & Defense |
| 50 | 2.782 | AI442332 | Metabolism |
| 51 | 2.781 | BI967711 | Transcription |
| 52 | 2.769 | BU926045 | Unclassified - Hypothetical Protein Supported by |
| | | | cDNA |
| 53 | 2.76 | BI317002 | Signal Transduction |
| 54 | 2.733 | AI442316 | Unclassified - Hypothetical Protein Supported by |
| | | | cDNA |
| 55 | 2.73 | BQ453808 | Metabolism |
| 56 | 2.682 | BE822092 | Unclassified - Hypothetical Protein Supported by |
| | | | cDNA |
| 57 | 2.682 | L22162.1 | Cell Structure |
| 58 | 2.637 | BI968208 | Secondary Metabolism |
| 59 | 2.59 | AW350127 | Cell Structure |
| 60 | 2.585 | BE659118 | No Homology to Known Proteins |
| 61 | 2.564 | BQ079907 | Unclassified - Hypothetical Protein NOT Supported by |
| | | | cDNA |
| 62 | 2.559 | AW101961 | Signal Transduction |
| 63 | 2.546 | AW156410 | No Homology to Known Proteins |
| 64 | 2.528 | BI893703 | Signal Transduction |
| 65 | 2.501 | BE658683 | No Homology to Known Proteins |
| 66 | 2.498 | BI700329 | Cell Structure |
| 67 | 2.484 | AW310721 | Secondary Metabolism |
| 68 | 2.455 | AW831390 | Signal Transduction |
| 69 | 2.437 | CF807071 | Unclassified - Hypothetical Protein Supported by |
| | | | cDNA |
| 70 | 2.428 | CD392418 | Transcription |
| 71 | 2.428 | BQ627652 | No Homology to Known Proteins |
| 72 | 2.403 | CK606530 | No Homology to Known Proteins |
| 73 | 2.387 | AW349594 | No Homology to Known Proteins |
| 74 | 2.376 | CA819279 | No Homology to Known Proteins |
| 75 | 2.373 | BU964918 | Transcription |
| 76 | 2.368 | BQ081294 | No Homology to Known Proteins |
| 77 | 2.367 | BG042115 | Unclassified - Hypothetical Protein Supported by |
| | | | cDNA |
| 78 | 2.363 | CD394504 | Cell Structure |
| 79 | 2.351 | BM271530 | No Homology to Known Proteins |
| 80 | 2.342 | BQ740576 | Signal Transduction |
| 81 | 2.335 | BU550606 | Protein Destination & Storage |
| 82 | 2.327 | CD406079 | Protein Destination & Storage |
| 83 | 2.314 | BG839320 | Signal Transduction |
| 84 | 2.312 | BE059411 | Cell Growth & Division |
| 85 | 2.289 | AW350946 | Unclassified - Hypothetical Protein NOT Supported by |
| | | | cDNA |
| 86 | 2.287 | BI426712 | Unclassified - Protein with Unknown Function |
| 87 | 2.272 | BQ785322 | Unclassified - Hypothetical Protein Supported by |
| | | | cDNA |
| 88 | 2.265 | BE059755 | No Homology to Known Proteins |
| 89 | 2.246 | CD412522 | Energy |
| 90 | 2.239 | AW310734 | Unclassified - Protein with Unknown Function |
| 91 | 2.234 | BU765206 | Transporter |
| 92 | 2.23 | BU764259 | Unclassified - Hypothetical Protein NOT Supported by |
| | | | cDNA |
| 93 | 2.227 | AI441130 | Signal Transduction |
| 94 | 2.196 | BQ298504 | Secondary Metabolism |
| 95 | 2.195 | BU547925 | No Homology to Known Proteins |
| 96 | 2.191 | CD399954 | Unclassified - Hypothetical Protein Supported by |
| | | | cDNA |
| 97 | 2.19 | AI495798 | Signal Transduction |
| 98 | 2.185 | CA938507 | Transcription |
| 99 | 2.178 | CF807977 | Metabolism |
| 100 | 2.178 | CD399954 | Unclassified - Hypothetical Protein Supported by |
| | | | cDNA |
| 101 | 2.154 | BQ610563 | Disease & Defense |
| 102 | 2.134 | AW350303 | Unclassified - Hypothetical Protein NOT Supported by |
| | | | cDNA |
| 103 | 2.117 | CF808939 | No Homology to Known Proteins |
| 104 | 2.104 | AW234479 | Unclassified - Hypothetical Protein NOT Supported by |
| | | | cDNA |
| 105 | 2.082 | AW598141 | No Homology to Known Proteins |
| 106 | 2.079 | BI968697 | No Homology to Known Proteins |
| 107 | 2.079 | BQ628831 | Secondary Metabolism |
| 108 | 2.075 | BQ629283 | Unclassified - Protein with Unknown Function |
| 109 | 2.073 | CF805767 | Signal Transduction |
| 110 | 2.067 | BM092432 | No Homology to Known Proteins |
| 111 | 2.026 | BI317557 | Disease & Defense |
| 112 | 2.016 | AW310476 | Cell Structure |
| 113 | 2.012 | AW164562 | Unclassified - Hypothetical Protein NOT Supported by |
| | | | cDNA |
| 114 | 2.005 | BG511492 | Signal Transduction |
| 115 | 0.5 | BG507699 | Metabolism |
| 116 | 0.5 | BQ453407 | Cell Structure |
| 117 | 0.499 | BG045916 | Transcription |
| 118 | 0.498 | BQ081678 | No Homology to Known Proteins |
| 119 | 0.497 | BM092634 | Cell Structure |
| 120 | 0.496 | BU965464 | Transporter |
| 121 | 0.495 | BU544060 | No Homology to Known Proteins |
| 122 | 0.494 | CA852250 | Transcription |
| 123 | 0.492 | CA782161 | Transporter |
| 124 | 0.491 | BE610836 | Cell Structure |
| 125 | 0.489 | BQ610190 | Unclassified - Hypothetical Protein NOT Supported by |
| | | | cDNA |
| 126 | 0.489 | BQ298780 | Protein Destination & Storage |
| 127 | 0.489 | CF808261 | Secondary Metabolism |
| 128 | 0.489 | BI786260 | Transcription |
| 129 | 0.488 | U50075.1 | Metabolism |
| 130 | 0.488 | CA852693 | No Homology to Known Proteins |
| 131 | 0.487 | AI416637 | Transcription |
| 132 | 0.486 | BM885600 | No Homology to Known Proteins |
| 133 | 0.485 | CD392747 | Transcription |
| 134 | 0.484 | BG726023 | Transcription |
| 135 | 0.484 | BI967418 | No Homology to Known Proteins |
| 136 | 0.483 | AW101438 | Secondary Metabolism |
| 137 | 0.482 | BU551014 | Unclassified - Hypothetical Protein Supported by |
| | | | cDNA |
| 138 | 0.481 | U41475.1 | Metabolism |
| 139 | 0.481 | U50075.1 | Metabolism |
| 140 | 0.481 | BF219584 | Secondary Metabolism |
| 141 | 0.479 | BE658252 | Unclassified - Hypothetical Protein Supported by |
| | | | cDNA |
| 142 | 0.479 | BM885538 | Unclassified - Hypothetical Protein Supported by |
| | | | cDNA |
| 143 | 0.478 | CD397107 | Unclassified - Hypothetical Protein Supported by |
| | | | cDNA |
| 144 | 0.478 | CA799081 | Unclassified - Hypothetical Protein NOT Supported by |
| | | | cDNA |
| 145 | 0.477 | CF805959 | Transcription |
| 146 | 0.475 | AW186006 | Protein Synthesis |
| 147 | 0.475 | BE058375 | Transcription |
| 148 | 0.475 | BG652533 | Unclassified - Hypothetical Protein Supported by |
| | | | cDNA |
| 149 | 0.473 | BG046201 | Transcription |
| 150 | 0.472 | BQ612831 | Signal Transduction |
| 151 | 0.472 | BI426008 | Unclassified - Hypothetical Protein NOT Supported by |
| | | | cDNA |
| 152 | 0.47 | CF809155 | Disease & Defense |
| 153 | 0.469 | BI321742 | Protein Destination & Storage |
| 154 | 0.468 | BM526951 | Unclassified - Hypothetical Protein Supported by |
| | | | cDNA |
| 155 | 0.468 | BU761195 | No Homology to Known Proteins |
| 156 | 0.467 | BQ452483 | Metabolism |
| 157 | 0.463 | AW350037 | Unclassified - Hypothetical Protein Supported by |
| | | | cDNA |
| 158 | 0.463 | CF806290 | Disease & Defense |
| 159 | 0.461 | BU764979 | Unclassified - Hypothetical Protein Supported by |
| | | | cDNA |
| 160 | 0.461 | BU549835 | No Homology to Known Proteins |
| 161 | 0.461 | CD401652 | Protein Destination & Storage |
| 162 | 0.46 | BF598958 | No Homology to Known Proteins |
| 163 | 0.458 | AI988066 | Unclassified - Hypothetical Protein NOT Supported by |
| | | | cDNA |
| 164 | 0.457 | CF806868 | Signal Transduction |
| 165 | 0.457 | AW472195 | Unclassified - Hypothetical Protein Supported by |
| | | | cDNA |
| 166 | 0.455 | BQ080041 | Disease & Defense |
| 167 | 0.454 | CF806624 | Unclassified - Hypothetical Protein NOT Supported by |
| | | | cDNA |
| 168 | 0.453 | AW598267 | No Homology to Known Proteins |
| 169 | 0.451 | AW760679 | Transcription |
| 170 | 0.449 | BM522940 | No Homology to Known Proteins |
| 171 | 0.447 | BI317776 | Transcription |
| 172 | 0.444 | BM085321 | Signal Transduction |
| 173 | 0.443 | BG047001 | No Homology to Known Proteins |
| 174 | 0.442 | BF066553 | Metabolism |
| 175 | 0.442 | BM143105 | Unclassified - Hypothetical Protein Supported by |
| | | | cDNA |
| 176 | 0.442 | BG511072 | Unclassified - Hypothetical Protein Supported by |
| | | | cDNA |
| 177 | 0.439 | BM308158 | No Homology to Known Proteins |
| 178 | 0.438 | BQ611806 | Metabolism |
| 179 | 0.437 | AI748012 | Protein Destination & Storage |
| 180 | 0.434 | BI968513 | Secondary Metabolism |
| 181 | 0.434 | AW279540 | Signal Transduction |
| 182 | 0.432 | BU577475 | Secondary Metabolism |
| 183 | 0.432 | CF807532 | No Homology to Known Proteins |
| 184 | 0.429 | AI960210 | Transporter |
| 185 | 0.429 | BF426048 | Unclassified - Hypothetical Protein Supported by |
| | | | cDNA |
| 186 | 0.428 | CA802431 | Unclassified - Hypothetical Protein Supported by |
| | | | cDNA |
| 187 | 0.427 | BI967182 | Secondary Metabolism |
| 188 | 0.425 | BE657895 | Unclassified - Hypothetical Protein Supported by |
| | | | cDNA |
| 189 | 0.425 | BI971147 | No Homology to Known Proteins |
| 190 | 0.425 | BE346777 | Transcription |
| 191 | 0.425 | BI498912 | Signal Transduction |
| 192 | 0.421 | CD408183 | Metabolism |
| 193 | 0.419 | BM527313 | Unclassified - Hypothetical Protein Supported by |
| | | | cDNA |
| 194 | 0.418 | BE800435 | Disease & Defense |
| 195 | 0.418 | BI973545 | No Homology to Known Proteins |
| 196 | 0.417 | BI472203 | Unclassified - Hypothetical Protein Supported by |
| | | | cDNA |
| 197 | 0.415 | CD404946 | Unclassified - Hypothetical Protein NOT Supported by |
| | | | cDNA |
| 198 | 0.415 | BQ080317 | Transcription |
| 199 | 0.414 | BE659400 | Unclassified - Protein with Unknown Function |
| 200 | 0.414 | BG155389 | Protein Destination & Storage |
| 201 | 0.413 | AW459859 | Signal Transduction |
| 202 | 0.411 | BG789983 | Secondary Metabolism |
| 203 | 0.409 | CD404723 | No Homology to Known Proteins |
| 204 | 0.402 | | |
| 205 | 0.399 | BU765857 | Protein Destination & Storage |
| 206 | 0.399 | AI965744 | Signal Transduction |
| 207 | 0.39 | BI970687 | Unclassified - Hypothetical Protein Supported by |
| | | | cDNA |
| 208 | 0.389 | BE821208 | Metabolism |
| 209 | 0.387 | BE329834 | Unclassified - Hypothetical Protein Supported by |
| | | | cDNA |
| 210 | 0.384 | AW350622 | Unclassified - Hypothetical Protein Supported by |
| | | | cDNA |
| 211 | 0.384 | BU549436 | Metabolism |
| 212 | 0.381 | BI971594 | No Homology to Known Proteins |
| 213 | 0.38 | AW733336 | Metabolism |
| 214 | 0.38 | CA819434 | No Homology to Known Proteins |
| 215 | 0.379 | CF807524 | Transporter |
| 216 | 0.378 | BG651846 | Metabolism |
| 217 | 0.377 | BU550168 | Signal Transduction |
| 218 | 0.376 | BE611084 | No Homology to Known Proteins |
| 219 | 0.373 | CD396298 | Energy |
| 220 | 0.372 | CD416539 | No Homology to Known Proteins |
| 221 | 0.372 | CA938391 | Transporter |
| 222 | 0.37 | BU551112 | Protein Destination & Storage |
| 223 | 0.37 | BU762686 | Metabolism |
| 224 | 0.367 | CD393602 | Transporter |
| 225 | 0.367 | BI892943 | Protein Destination & Storage |
| 226 | 0.366 | BE822845 | Energy |
| 227 | 0.366 | AW567763 | No Homology to Known Proteins |
| 228 | 0.362 | BG363704 | No Homology to Known Proteins |
| 229 | 0.348 | BI969715 | Metabolism |
| 230 | 0.347 | BI971156 | Signal Transduction |
| 231 | 0.345 | BQ628677 | No Homology to Known Proteins |
| 232 | 0.344 | BI322039 | No Homology to Known Proteins |
| 233 | 0.343 | BE820997 | Cell Structure |
| 234 | 0.343 | AW317461 | Protein Destination & Storage |
| 235 | 0.343 | BU083387 | Transcription |
| 236 | 0.341 | BQ298381 | Disease & Defense |
| 237 | 0.341 | AW309251 | Transcription |
| 238 | 0.339 | BU547906 | Transcription |
| 239 | 0.336 | CF808516 | Metabolism |
| 240 | 0.334 | BU765815 | Transcription |
| 241 | 0.332 | BE059054 | Metabolism |
| 242 | 0.331 | BI785710 | Transporter |
| 243 | 0.328 | BI968944 | Secondary Metabolism |
| 244 | 0.326 | CD400596 | Transcription |
| 245 | 0.325 | BU546115 | Secondary Metabolism |
| 246 | 0.321 | BQ079657 | Protein Destination & Storage |
| 247 | 0.318 | BG156739 | No Homology to Known Proteins |
| 248 | 0.318 | CD414199 | Metabolism |
| 249 | 0.313 | BE801134 | Metabolism |
| 250 | 0.312 | BM084966 | Transcription |
| 251 | 0.312 | AF004806.1 | Protein Destination & Storage |
| 252 | 0.311 | BQ628998 | Disease & Defense |
| 253 | 0.308 | CA782582 | No Homology to Known Proteins |
| 254 | 0.308 | AI443109 | Signal Transduction |
| 255 | 0.305 | BI971626 | Unclassified - Hypothetical Protein NOT Supported by |
| | | | cDNA |
| 256 | 0.305 | | |
| 257 | 0.3 | BG237979 | No Homology to Known Proteins |
| 258 | 0.295 | BF010276 | Transcription |
| 259 | 0.292 | AF109720.1 | Protein Destination & Storage |
| 260 | 0.29 | BQ786048 | Disease & Defense |
| 261 | 0.288 | BE802287 | Metabolism |
| 262 | 0.287 | CD398445 | No Homology to Known Proteins |
| 263 | 0.286 | BU550316 | Cell Structure |
| 264 | 0.285 | CA937360 | Transcription |
| 265 | 0.274 | AW568984 | No Homology to Known Proteins |
| 266 | 0.273 | AB004271.1 | Metabolism |
| 267 | 0.272 | AI442669 | Protein Destination & Storage |
| 268 | 0.261 | AW351012 | No Homology to Known Proteins |
| 269 | 0.25 | BG157024 | No Homology to Known Proteins |
| 270 | 0.247 | CD408184 | Protein Destination & Storage |
| 271 | 0.247 | BE822538 | Unclassified - Hypothetical Protein Supported by |
| | | | cDNA |
| 272 | 0.246 | AW311637 | Metabolism |
| 273 | 0.239 | AW349832 | No Homology to Known Proteins |
| 274 | 0.238 | BM886862 | Transcription |
| 275 | 0.229 | BE806784 | Unclassified - Hypothetical Protein NOT Supported by |
| | | | cDNA |
| 276 | 0.219 | CA937360 | Transcription |
| 277 | 0.219 | BE820777 | Unclassified - Hypothetical Protein Supported by |
| | | | cDNA |
| 278 | 0.207 | CA819977 | Unclassified - Protein with Unknown Function |
| 279 | 0.202 | CF806004 | Protein Destination & Storage |
| 280 | 0.192 | BE822501 | Unclassified - Hypothetical Protein Supported by |
| | | | cDNA |
| 281 | 0.191 | CF807524 | Transporter |
| 282 | 0.17 | CK605778 | Metabolism |
| 283 | 0.17 | CF807204 | No Homology to Known Proteins |
| 284 | 0.168 | CK605778 | Metabolism |
| 285 | 0.163 | AW759183 | Protein Destination & Storage |
| 286 | 0.147 | CD418313 | Transporter |
| 287 | 0.141 | CD408184 | Protein Destination & Storage |
| 288 | 0.129 | CF807524 | Transporter |
| 289 | 0.123 | BE820822 | Unclassified - Hypothetical Protein Supported by |
| | | | cDNA |
| 290 | 0.1 | CF806537 | Disease & Defense |

| ID | Sub-Family | Gene Family | Additional Description |
|---|---|---|---|
| 1 | | | |
| 2 | | | |
| 3 | Protein Kinase | | |
| 4 | Cytochrome | CYP93D | Cytochrome P450 CYP2 subfamily |
| | P450 | | |
| 5 | | | |
| 6 | | | |
| 7 | | | |
| 8 | Storage Protein | | |
| 9 | | | |
| 10 | | | Haloacid dehalogenase-like hydrolase |
| 11 | | | |
| 12 | | | Aldehyde reductase |
| 13 | Trehalose | | Trehalose-6-phosphate synthase |
| | Biosynthesis | | component TPS1 and related subunits |
| 14 | | | |
| 15 | | | Gelatinase A and related matrix |
| | | | metalloproteases |
| 16 | | | Copper chaperone |
| 17 | | | Copper chaperone |
| 18 | | | Copper chaperone |
| 19 | | | |
| 20 | | | |
| 21 | | | |
| 22 | | | |
| 23 | | | Branched chain aminotransferase |
| | | | BCAT1, pyridoxal phosphate enzymes |
| | | | type IV superfamily |
| 24 | | | |
| 25 | Protein Kinase | AtCIPKs | Serine/threonine protein kinase |
| 26 | | | Branched chain aminotransferase |
| | | | BCAT1, pyridoxal phosphate enzymes |
| | | | type IV superfamily |
| 27 | | | |
| 28 | Phospholipid | | Glycosylphosphatidylinositol-specific |
| | Biosynthesis | | phospholipase C |
| 29 | | | Thioredoxin |
| 30 | | | Copper chaperone |
| 31 | | | |
| 32 | Protein Kinase | | |
| 33 | | | |
| 34 | | | |
| 35 | Protein Kinase | AtCIPKs | Serine/threonine protein kinase |
| 36 | | | |
| 37 | | | |
| 38 | Transcription | AP2/EREBP | |
| | Factor | | |
| 39 | | | |
| 40 | Protein Kinase | | |
| 41 | | | |
| 42 | Protein Kinase | | |
| 43 | | | |
| 44 | Calcium Binding | | |
| 45 | | | |
| 46 | | | |
| 47 | Protein Kinase | | |
| 48 | | | Aquaporin (major intrinsic protein |
| | | | family) |
| 49 | | | |
| 50 | | | Lipid phosphate phosphatase and |
| | | | related enzymes of the PAP2 family |
| 51 | Transcription | Zinc Finger | FOG: Zn-finger |
| | Factor | | |
| 52 | | | |
| 53 | Protein Kinase | | |
| 54 | | | |
| 55 | | | Predicted lipase |
| 56 | | | |
| 57 | | | |
| 58 | Flavonoid | | |
| | Biosynthesis | | |
| 59 | | | |
| 60 | | | |
| 61 | | | |
| 62 | Protein Kinase | | Serine/threonine protein kinase |
| 63 | | | |
| 64 | Protein Kinase | | Serine/threonine protein kinase |
| 65 | | | |
| 66 | | | |
| 67 | | | |
| 68 | Protein Kinase | | |
| 69 | | | |
| 70 | Transcription | AP2/EREBP | |
| | Factor | | |
| 71 | | | |
| 72 | | | |
| 73 | | | |
| 74 | | | |
| 75 | Transcription | AP2/EREBP | |
| | Factor | | |
| 76 | | | |
| 77 | | | |
| 78 | | | Beta tubulin |
| 79 | | | |
| 80 | Protein Kinase | | |
| 81 | | | Molecular chaperone (HSP90 family) |
| 82 | | | Serine carboxypeptidases (lysosomal |
| | | | cathepsin A) |
| 83 | Protein Kinase | | Serine/threonine protein kinase |
| 84 | | | Microtubule-associated protein |
| | | | essential for anaphase spindle |
| | | | elongation |
| 85 | | | |
| 86 | | | |
| 87 | | | |
| 88 | | | |
| 89 | | | |
| 90 | Hormone | | Voltage-gated shaker-like K+ channel, |
| | Induced | | subunit beta/KCNAB |
| 91 | | | |
| 92 | | | |
| 93 | Protein Kinase | | Serine/threonine protein kinase |
| 94 | | | Hydroxyindole-O-methyltransferase |
| | | | and related SAM-dependent |
| | | | methyltransferases |
| 95 | | | |
| 96 | | | |
| 97 | Protein Kinase | AtCIPKs | Serine/threonine protein kinase |
| 98 | Transcription | Zinc Finger | GATA-4/5/6 transcription factors |
| | Factor | | |
| 99 | | | Branched chain aminotransferase |
| | | | BCAT1, pyridoxal phosphate enzymes |
| | | | type IV superfamily |
| 100 | | | |
| 101 | | | |
| 102 | | | |
| 103 | | | |
| 104 | | | |
| 105 | | | |
| 106 | | | |
| 107 | Flavonoid | | Flavonol reductase/cinnamoyl-CoA |
| | Biosynthesis | | reductase |
| 108 | | | |
| 109 | Calcium Binding | | Calmodulin and related proteins (EF- |
| | | | Hand superfamily) |
| 110 | | | |
| 111 | | | Ferric reductase, NADH/NADPH |
| | | | oxidase and related proteins |
| 112 | | | |
| 113 | | | |
| 114 | MAPKK | | Mitogen-activated protein kinase |
| | | | kinase (MAP2K) |
| 115 | | | |
| 116 | | | Beta-glucosidase, lactase |
| | | | phlorizinhydrolase, and related |
| | | | proteins |
| 117 | Transcription | MYB | Transcription factor, Myb superfamily |
| | Factor | | |
| 118 | | | |
| 119 | | | |
| 120 | Auxin Transport | | |
| 121 | | | |
| 122 | Transcription | Unclassified | |
| | Factor | | |
| 123 | | | |
| 124 | | | |
| 125 | | | AAA+-type ATPase |
| 126 | | | |
| 127 | Ethylene | | Iron/ascorbate family oxidoreductases |
| | Biosynthesis | | |
| 128 | Transcription | Unclassified | GATA-4/5/6 transcription factors |
| | Factor | | |
| 129 | | | |
| 130 | | | |
| 131 | Transcription | Homeodomain | Transcription factor MEIS1 and related |
| | Factor | | HOX domain proteins |
| 132 | | | |
| 133 | Transcription | Basic Helix- | |
| | Factor | Loop-Helix | |
| | | (bHLH) | |
| 134 | Transcription | Homeodomain | Transcription factor MEIS1 and related |
| | Factor | | HOX domain proteins |
| 135 | | | |
| 136 | GA Biosynthesis | | Iron/ascorbate family oxidoreductases |
| 137 | | | |
| 138 | | | Phosphoinositide-specific |
| | | | phospholipase C |
| 139 | | | |
| 140 | | | |
| 141 | | | |
| 142 | | | |
| 143 | | | |
| 144 | | | |
| 145 | Transcription | Zinc Finger | FOG: Zn-finger |
| | Factor | | |
| 146 | | | Translation initiation factor 5A (eIF- |
| | | | 5A) |
| 147 | Transcription | MYB | Transcription factor, Myb superfamily |
| | Factor | | |
| 148 | | | |
| 149 | Transcription | Unclassified | Zuotin and related molecular |
| | Factor | | chaperones (DnaJ superfamily), |
| | | | contains DNA-binding domains |
| 150 | MAPKKK | MEKK | MEKK and related serine/threonine |
| | | | protein kinases |
| 151 | | | Predicted dehydrogenase |
| 152 | | | Purple (tartrate-resistant) acid |
| | | | phosphatase |
| 153 | | | Ubiquitin-protein ligase |
| 154 | | | Leucine rich repeat proteins, some |
| | | | proteins contain F-box |
| 155 | | | |
| 156 | | | UDP-glucuronosyl and UDP-glucosyl |
| | | | transferase |
| 157 | | | |
| 158 | | | |
| 159 | | | GTPase Rab18, small G protein |
| | | | superfamily |
| 160 | | | |
| 161 | | | Glutathione S-transferase |
| 162 | | | |
| 163 | | | UDP-galactose transporter related |
| | | | protein |
| 164 | Unclassified | | |
| 165 | | | |
| 166 | | | |
| 167 | | | |
| 168 | | | |
| 169 | Transcription | Basic Helix- | |
| | Factor | Loop-Helix | |
| | | (bHLH) | |
| 170 | | | |
| 171 | Transcription | WRKY | |
| | Factor | | |
| 172 | Phytochrome | | eIF-2alpha kinase PEK/EIF2AK3 |
| 173 | | | |
| 174 | | | Transketolase |
| 175 | | | |
| 176 | | | Galactosyltransferases |
| 177 | | | |
| 178 | | | Flavin-containing monooxygenase |
| 179 | | | |
| 180 | Ethylene | | Iron/ascorbate family oxidoreductases |
| | Biosynthesis | | |
| 181 | Protein | | Serine/threonine protein phosphatase |
| | Phosphatase | | |
| 182 | Ethylene | | Iron/ascorbate family oxidoreductases |
| | Biosynthesis | | |
| 183 | | | |
| 184 | | | Aquaporin (major intrinsic protein |
| | | | family) |
| 185 | | | |
| 186 | | | NADH pyrophosphatase I of the Nudix |
| | | | family of hydrolases |
| 187 | Ethylene | | Iron/ascorbate family oxidoreductases |
| | Biosynthesis | | |
| 188 | | | |
| 189 | | | |
| 190 | Transcription | MYB | Transcription factor, Myb superfamily |
| | Factor | | |
| 191 | Calcium Binding | Calcium | |
| | | Dependent | |
| | | Protein | |
| | | Kinase | |
| 192 | | | |
| 193 | | | |
| 194 | | | |
| 195 | | | |
| 196 | | | |
| 197 | | | Uncharacterized conserved protein |
| 198 | Transcription | Zinc Finger | |
| | Factor | | |
| 199 | | | Acyl-CoA reductase |
| 200 | | | Ubiquitin fusion-degradation protein |
| 201 | Unclassified | | Lanthionine synthetase C-like protein 1 |
| 202 | | | Hydroxyindole-O-methyltransferase |
| | | | and related SAM-dependent |
| | | | methyltransferases |
| 203 | | | |
| 204 | | | |
| 205 | Ubiquitin | | FOG: Predicted E3 ubiquitin ligase |
| | Degradation | | |
| 206 | Protein Kinase | | |
| 207 | | | |
| 208 | | | Glutamate decarboxylase and related |
| | | | proteins |
| 209 | | | |
| 210 | | | |
| 211 | | | Long-chain acyl-CoA synthetases |
| | | | (AMP-forming) |
| 212 | | | |
| 213 | Cytochrome | CYP716A | Cytochrome P450 |
| | P450 | | CYP4/CYP19/CYP26 subfamilies |
| 214 | | | |
| 215 | | | |
| 216 | | | Beta-galactosidase |
| 217 | Protein Kinase | | Putative serine/threonine protein kinase |
| 218 | | | |
| 219 | | | |
| 220 | | | |
| 221 | | | |
| 222 | | | Ubiquitin-protein ligase |
| 223 | | | |
| 224 | | | |
| 225 | | | FKBP-type peptidyl-prolyl cis-trans |
| | | | isomerase |
| 226 | | | Ferric reductase, NADH/NADPH |
| | | | oxidase and related proteins |
| 227 | | | |
| 228 | | | |
| 229 | | | Hydroxyindole-O-methyltransferase |
| | | | and related SAM-dependent |
| | | | methyltransferases |
| 230 | Protein Kinase | | |
| 231 | | | |
| 232 | | | |
| 233 | | | O-methyltransferase |
| 234 | | | Serine carboxypeptidases |
| 235 | Transcription | Basic Helix- | |
| | Factor | Loop-Helix | |
| | | (bHLH) | |
| 236 | | | |
| 237 | Transcription | Basic Helix- | |
| | Factor | Loop-Helix | |
| | | (bHLH) | |
| 238 | Transcription | NAC Domain | |
| | Factor | | |
| 239 | | | Reductases with broad range of |
| | | | substrate specificities |
| 240 | Transcription | Homeodomain | Transcription factor MEIS1 and related |
| | Factor | | HOX domain proteins |
| 241 | | | |
| 242 | | | Glutaredoxin-related protein |
| 243 | | | NADH:flavin oxidoreductase/12- |
| | | | oxophytodienoate reductase |
| 244 | Transcription | MYB | |
| | Factor | | |
| 245 | | | Iron/ascorbate family oxidoreductases |
| 246 | | | Serine carboxypeptidases |
| 247 | | | |
| 248 | | | Glycine cleavage system H protein |
| | | | (lipoate-binding) |
| 249 | | | |
| 250 | Transcription | Basic Leucine | |
| | Factor | Zipper (bZIP) | |
| 251 | Storage Protein | | Reticulon |
| 252 | | | Soluble epoxide hydrolase |
| 253 | | | |
| 254 | Unclassified | | Lanthionine synthetase C-like protein 1 |
| 255 | | | |
| 256 | | | |
| 257 | | | |
| 258 | Transcription | MYB | Transcription factor, Myb superfamily |
| | Factor | | |
| 259 | Storage Protein | | |
| 260 | | | Predicted metallothionein |
| 261 | | | Uncharacterized conserved protein |
| 262 | | | |
| 263 | | | |
| 264 | Transcription | Basic Helix- | |
| | Factor | Loop-Helix | |
| | | (bHLH) | |
| 265 | | | |
| 266 | | | |
| 267 | | | FOG: Predicted E3 ubiquitin ligase |
| 268 | | | |
| 269 | | | |
| 270 | | | |
| 271 | | | Endoplasmic reticulum protein EP58, |
| | | | contains filamin rod domain and KDEL |
| | | | motif |
| 272 | | | |
| 273 | | | |
| 274 | Transcription | NAC Domain | |
| | Factor | | |
| 275 | | | |
| 276 | Transcription | Basic Helix- | |
| | Factor | Loop-Helix | |
| | | (bHLH) | |
| 277 | | | |
| 278 | | | |
| 279 | Storage Protein | | Uncharacterized conserved protein |
| 280 | | | |
| 281 | | | |
| 282 | | | |
| 283 | | | |
| 284 | | | |
| 285 | | | Ubiquitin-protein ligase |
| 286 | | | |
| 287 | | | |
| 288 | | | |
| 289 | | | |
| 290 | | | |

TABLE 9

| | | | Soybean_Gen | | | Gene | |
|---|---|---|---|---|---|---|---|
| ID | Gene Name | Fold Change | Bank_Acc | Functional Category | Sub-Family | Family | Additional Description |
| 1 | Gma.10892.1.S1_a_at | 14.15 | BG839570 | Metabolism | | | Predicted carbonic anhydrase involved in |
| | | | | | | | protection against oxidative damage |
| 2 | Gma.10921.1.S1_s_at | 8.989 | BQ611250 | No Homology to Known Proteins | | | |
| 3 | Gma.12356.1.A1_at | 8.807 | CD397798 | Unclassified - Hypothetical Protein | | | |
| | | | | NOT Supported by cDNA | | | |
| 4 | GmaAffx.42583.1.A1_at | 8.466 | BG154898 | Transporter | Inorganic | Fe(II) | Fe2+/Zn2+ regulated transporter |
| | | | | | Solute | transporter | |
| | | | | | Cotransporters | isolog family | |
| 5 | Gma.5689.3.S1_s_at | 8.444 | BE657645 | Protein Destination & Storage | | | Gelatinase A and related matrix metalloproteases |
| 6 | GmaAffx.14016.1.S1_at | 8.082 | BE824450 | No Homology to Known Proteins | | | |
| 7 | GmaAffx.9605.1.S1_at | 5.481 | BM091603 | No Homology to Known Proteins | | | |
| 8 | Gma.964.1.A1_at | 5.456 | BE824146 | Metabolism | | | Tyrosine aminotransferase |
| 9 | Gma.4058.1.S1_at | 5.299 | AW310338 | Transporter | | | Predicted transporter (major facilitator superfamily) |
| 10 | Gma.17789.1.S1_at | 5.221 | BG511704 | Unclassified - Hypothetical Protein | | | |
| | | | | Supported by cDNA | | | |
| 11 | Gma.1221.1.S1_s_at | 5.045 | L23854.1 | Metabolism | | | Cytochrome b5 |
| 11 | Gma.1221.1.S1_s_at | 5.045 | L23854.1 | Metabolism | | | NADH-cytochrome b-5 reductase |
| 11 | Gma.1221.1.S1_s_at | 5.045 | L23854.1 | Metabolism | | | Sulfite oxidase, molybdopterin-binding component |
| 12 | GmaAffx.19116.1.S1_at | 4.956 | BI968419 | Signal Transduction | Protein | | |
| | | | | | Kinase | | |
| 13 | GmaAffx.9866.1.S1_at | 4.784 | BM524432 | Metabolism | | | |
| 14 | Gma.5139.1.S1_at | 4.589 | AF243376.1 | Secondary Metabolism | | | Glutathione S-transferase |
| 15 | Gma.8416.1.S1_at | 3.823 | U13987.1 | Metabolism | | | Cytochrome b5 |
| 15 | Gma.8416.1.S1_at | 3.823 | U13987.1 | Metabolism | | | NADH-cytochrome b-5 reductase |
| 15 | Gma.8416.1.S1_at | 3.823 | U13987.1 | Metabolism | | | Sulfite oxidase, molybdopterin-binding component |
| 16 | Gma.6266.1.A1_at | 3.609 | CD413418 | Unclassified - Protein with | | | |
| | | | | Unknown Function | | | |
| 17 | GmaAffx.3905.3.S1_at | 3.456 | BG047142 | Unclassified - Protein with | | | |
| | | | | Unknown Function | | | |
| 18 | Gma.7332.1.S1_at | 3.119 | BI945636 | Unclassified - Hypothetical Protein | | | |
| | | | | Supported by cDNA | | | |
| 19 | Gma.4701.2.A1_a_at | 2.971 | BM526048 | Energy | | | Thioredoxin |
| 20 | GmaAffx.3369.1.S1_at | 2.835 | BU548983 | Cell Structure | | | |
| 21 | GmaAffx.67477.2.S1_at | 2.756 | AW509184 | Cell Structure | | | |
| 22 | Gma.3581.2.S1_a_at | 2.671 | CD398783 | Unclassified - Hypothetical Protein | | | Ca2+-dependent lipid-binding protein |
| | | | | NOT Supported by cDNA | | | CLB1/vesicle protein vp115/Granuphilin A, contains C2 |
| | | | | | | | domain |
| 23 | GmaAffx.90960.1.A1_s_at | 2.664 | CF806685 | Signal Transduction | Protein | | |
| | | | | | Kinase | | |
| 24 | GmaAffx.53845.1.S1_at | 2.63 | BG406552 | Transporter | | | Sulfate/bicarbonate/oxalate exchanger SAT-1 and |
| | | | | | | | related transporters (SLC26 family) |
| 25 | Gma.17321.1.S1_at | 2.572 | AW508844 | Metabolism | | | Tyrosine aminotransferase |
| 26 | Gma.7301.1.S1_at | 2.472 | BM528250 | Disease & Defense | | | Gamma-glutamyltransferase |
| 27 | GmaAffx.73185.1.S1_at | 2.442 | BI701903 | No Homology to Known Proteins | | | |
| 28 | Gma.3639.1.S1_at | 2.322 | AB083030.1 | Protein Destination & Storage | Storage | | |
| | | | | | Protein | | |
| 29 | Gma.184.1.S1_at | 2.279 | AW101261 | Unclassified - Hypothetical Protein | | | |
| | | | | Supported by cDNA | | | |
| 30 | GmaAffx.62542.1.S1_at | 2.266 | BE659136 | Unclassified - Hypothetical Protein | | | |
| | | | | NOT Supported by cDNA | | | |
| 31 | Gma.7608.1.S1_at | 2.247 | BU549027 | Unclassified - Hypothetical Protein | | | RNA-binding protein Sam68 and related KH |
| | | | | NOT Supported by cDNA | | | domain proteins |
| 32 | GmaAffx.6571.1.S1_at | 2.238 | CD403804 | Metabolism | | | UDP-glucuronosyl and UDP-glucosyl transferase |
| 33 | GmaAffx.40114.1.A1_at | 2.199 | BE661587 | Unclassified - Hypothetical Protein | | | |
| | | | | Supported by cDNA | | | |
| 34 | Gma.16521.1.S1_at | 2.185 | BE609300 | Unclassified - Hypothetical Protein | | | Uncharacterized conserved protein |
| | | | | Supported by cDNA | | | |
| 35 | Gma.16832.1.S1_s_at | 2.168 | L22029.1 | No Homology to Known Proteins | | | |
| 36 | GmaAffx.90947.1.S1_at | 2.161 | CF806672 | Unclassified - Protein with | | | |
| | | | | Unknown Function | | | |
| 37 | Gma.17816.1.A1_at | 2.151 | BI970976 | Disease & Defense | | | |
| 38 | Gma.5483.1.S1_s_at | 2.144 | CD414581 | Transcription | Transcription | Basic | |
| | | | | | Factor | Leucine | |
| | | | | | | Zipper | |
| | | | | | | (bZIP) | |
| 39 | GmaAffx.86059.1.S1_at | 2.135 | BF598759 | No Homology to Known Proteins | | | |
| 40 | GmaAffx.934.1.S1_at | 2.132 | BG046802 | No Homology to Known Proteins | | | |
| 41 | Gma.5124.1.S1_at | 2.128 | BI971079 | Protein Destination & Storage | | | |
| 42 | Gma.4374.1.S1_at | 2.08 | BM178538 | Metabolism | Cytochrome | CYP76C | Cytochrome P450 CYP2 subfamily |
| | | | | | P450 | | |
| 43 | GmaAffx.6.1.S1_at | 2.07 | AW348562 | No Homology to Known Proteins | | | |
| 44 | Gma.12212.1.A1_s_at | 2.053 | CD396776 | No Homology to Known Proteins | | | |
| 45 | Gma.3136.1.S1_at | 2.04 | BG839241 | Metabolism | Cytochrome | CYP82C | Cytochrome P450 CYP2 subfamily |
| | | | | | P450 | | |
| 46 | Gma.2194.1.S1_at | 2.038 | BE657352 | Unclassified - Hypothetical Protein | | | |
| | | | | Supported by cDNA | | | |
| 47 | Gma.7474.1.A1_at | 2.009 | BU547968 | Unclassified - Hypothetical Protein | | | |
| | | | | Supported by cDNA | | | |
| 48 | Gma.12641.1.A1_at | 0.5 | CD393495 | No Homology to Known Proteins | | | |
| 49 | GmaAffx.32345.1.A1_s_at | 0.499 | BI970277 | Cell Structure | | | |
| 50 | Gma.16910.1.S1_at | 0.498 | BU549219 | Unclassified - Protein with | | | |
| | | | | Unknown Function | | | |
| 51 | Gma.11665.2.A1_at | 0.498 | BU545959 | No Homology to Known Proteins | | | |
| 52 | GmaAffx.9400.2.S1_at | 0.497 | BI945484 | Signal Transduction | Protein | | Glycerophosphoryl diester phosphodiesterase |
| | | | | | Kinase | | |
| 52 | GmaAffx.9400.2.S1_at | 0.497 | BI945484 | Signal Transduction | Protein | | Serine/threonine protein kinase |
| | | | | | Kinase | | |
| 53 | Gma.2548.1.S1_at | 0.496 | BE821472 | Unclassified - Hypothetical Protein | | | |
| | | | | Supported by cDNA | | | |
| 54 | Gma.5929.1.S1_at | 0.496 | BQ610446 | No Homology to Known Proteins | | | |
| 55 | GmaAffx.60958.1.S1_at | 0.494 | BG551038 | Transcription | Transcription | Basic Helix- | |
| | | | | | Factor | Loop-Helix | |
| | | | | | | (bHLH) | |
| 56 | GmaAffx.37484.1.S1_at | 0.494 | BG651852 | Unclassified - Protein with | | | Predicted membrane protein, contains DoH and |
| | | | | Unknown Function | | | Cytochrome b-561/ferric reductase |
| | | | | | | | transmembrane domains |
| 57 | GmaAffx.81585.1.S1_at | 0.493 | BM187887 | Unclassified - Hypothetical Protein | | | Copper chaperone |
| | | | | NOT Supported by cDNA | | | |
| 58 | GmaAffx.78599.1.A1_at | 0.493 | CA784082 | No Homology to Known Proteins | | | |
| 59 | GmaAffx.43774.1.A1_at | 0.492 | AW349173 | Cell Structure | | | |
| 60 | Gma.1704.1.S1_at | 0.492 | AW349971 | Metabolism | | | Glutaredoxin and related proteins |
| 61 | GmaAffx.3697.1.S1_at | 0.492 | BE820647 | Disease & Defense | | | |
| 62 | GmaAffx.72479.1.S1_at | 0.491 | BG043825 | Transcription | Transcription | AP2/EREBP | Uncharacterized conserved protein |
| | | | | | Factor | | |
| 63 | GmaAffx.47228.1.S1_at | 0.491 | BU761053 | Transporter | | | |
| 64 | Gma.15907.2.S1_at | 0.491 | BE329550 | Signal Transduction | Unclassified | | FOG: Leucine rich repeat |
| 65 | PsAffx.psHB026xH01f_s_at | 0.49 | | | | | |
| 66 | Gma.13361.1.S1_at | 0.49 | AW309932 | Unclassified - Protein with | | | |
| | | | | Unknown Function | | | |
| 67 | Gma.11195.1.S1_at | 0.49 | CD417406 | Transporter | | | Apolipoprotein D/Lipocalin |
| 68 | Gma.4680.1.A1_s_at | 0.489 | AW309707 | Unclassified - Protein with | | | |
| | | | | Unknown Function | | | |
| 69 | GmaAffx.84795.1.S1_at | 0.489 | BG046191 | Unclassified - Hypothetical Protein | | | |
| | | | | NOT Supported by cDNA | | | |
| 70 | Gma.9013.1.S1_at | 0.488 | AF243368.1 | Disease & Defense | | | Glutathione S-transferase |
| 71 | GmaAffx.71589.1.S1_at | 0.487 | BE020161 | Cell Growth & Division | | | |
| 72 | Gma.8946.1.S1_at | 0.487 | BQ453813 | Transcription | Transcription | NAC | |
| | | | | | Factor | Domain | |
| 73 | GmaAffx.93189.1.S1_at | 0.487 | CF808914 | Metabolism | | | Carbon-nitrogen hydrolase |
| 74 | GmaAffx.75219.1.S1_at | 0.486 | BE020773 | Unclassified - Hypothetical Protein | | | Fasciclin and related adhesion glycoproteins |
| | | | | NOT Supported by cDNA | | | |
| 75 | Gma.10896.3.A1_at | 0.486 | BI316837 | Metabolism | | | Aldehyde dehydrogenase |
| 76 | GmaAffx.83564.1.S1_at | 0.485 | BU578309 | No Homology to Known Proteins | | | |
| 77 | GmaAffx.40086.1.S1_at | 0.485 | BE659815 | Unclassified - Hypothetical Protein | | | |
| | | | | Supported by cDNA | | | |
| 78 | Gma.6129.2.S1_s_at | 0.484 | AW186006 | Protein Synthesis | | | Translation initiation factor 5A (elF-5A) |
| 79 | Gma.1715.1.S1_at | 0.483 | BE191618 | Metabolism | | | |
| 80 | GmaAffx.72986.1.S1_at | 0.483 | BI317536 | Metabolism | | | Inositol polyphosphate 5-phosphatase and related |
| | | | | | | | proteins |
| 81 | GmaAffx.89049.1.S1_at | 0.483 | CK606168 | Intracellular Traffic | | | Annexin |
| 82 | GmaAffx.64807.1.S1_at | 0.483 | AW201972 | No Homology to Known Proteins | | | |
| 83 | GmaAffx.26967.1.S1_at | 0.482 | BE022958 | Unclassified - Hypothetical Protein | | | |
| | | | | Supported by cDNA | | | |
| 84 | Gma.2540.2.S1_s_at | 0.482 | BI787366 | No Homology to Known Proteins | | | |
| 85 | GmaAffx.28578.1.S1_at | 0.482 | BQ094224 | Disease & Defense | | | |
| 86 | GmaAffx.90331.1.S1_s_at | 0.482 | CF806056 | Disease & Defense | | | |
| 87 | GmaAffx.68754.1.S1_at | 0.481 | BE474746 | Secondary Metabolism | | | Hydroxyindole-O-methyltransferase and related |
| | | | | | | | SAM-dependent methyltransferases |
| 88 | Gma.5889.1.S1_at | 0.481 | BQ610202 | Unclassified - Hypothetical Protein | | | Endoplasmic reticulum protein EP58, contains filamin rod |
| | | | | NOT Supported by cDNA | | | domain and KDEL motif |
| 89 | Gma.1137.2.S1_x_at | 0.481 | CA852403 | Metabolism | | | |
| 90 | Gma.3591.1.S1_x_at | 0.481 | BE440266 | Secondary Metabolism | Ethylene | | Iron/ascorbate family oxidoreductases |
| | | | | | Biosynthesis | | |
| 91 | Gma.7970.1.S1_at | 0.481 | BI699189 | No Homology to Known Proteins | | | |
| 92 | GmaAffx.28539.1.S1_at | 0.481 | BQ080184 | Disease & Defense | | | Ca2+-independent phospholipase A2 |
| 93 | GmaAffx.61311.1.S1_at | 0.48 | CA936951 | Transporter | | | Amino acid transporters |
| 94 | GmaAffx.30428.1.S1_at | 0.48 | BE824410 | Protein Destination & Storage | | | Molecular chaperones HSP70/HSC70.HSP70 |
| | | | | | | | superfamily |
| 95 | Gma.16585.1.S1_x_at | 0.48 | CA850597 | Metabolism | | | Predicted carbonic anhydrase involved in |
| | | | | | | | protection against oxidative damage |
| 96 | Gma.8160.1.S1_at | 0.48 | BI787214 | Unclassified - Hypothetical Protein | | | |
| | | | | NOT Supported by cDNA | | | |
| 97 | GmaAffx.87670.1.S1_at | 0.48 | BQ452640 | Unclassified - Hypothetical Protein | | | |
| | | | | NOT Supported by cDNA | | | |
| 98 | Gma.728.1.A1_at | 0.479 | BE658779 | No Homology to Known Proteins | | | |
| 99 | Gma.4120.1.A1_at | 0.479 | AW310714 | Unclassified - Protein with | | | |
| | | | | Unknown Function | | | |
| 100 | Gma.6630.1.S1_s_at | 0.478 | BG652851 | Unclassified - Hypothetical Protein | | | |
| | | | | Supported by cDNA | | | |
| 101 | GmaAffx.81584.1.S1_at | 0.478 | BI426969 | Disease & Defense | | | |
| 102 | Gma.7040.1.A1_at | 0.478 | BE346424 | Transcription | Transcription | AP2/EREBP | |
| | | | | | Factor | | |
| 103 | GmaAffx.69076.1.S1_at | 0.477 | BM525610 | Unclassified - Hypothetical Protein | | | |
| | | | | NOT Supported by cDNA | | | |
| 104 | GmaAffx.53099.1.S1_at | 0.477 | BG043340 | Unclassified - Hypothetical Protein | | | Alpha-L-fucosidase |
| | | | | NOT Supported by cDNA | | | |
| 105 | Gma.8240.1.S1_at | 0.476 | BM092761 | Secondary Metabolism | | | Iron/ascorbate family oxidoreductases |
| 106 | GmaAffx.30665.1.A1_at | 0.476 | BQ453743 | Signal Transduction | Protein | | Serine/threonine protein kinase |
| | | | | | Kinase | | |
| 107 | Gma.16526.1.S1_at | 0.475 | CD407998 | Protein Destination & Storage | | | Serine carboxypeptidases (lysosomal cathepsin |
| | | | | | | | A) |
| 108 | GmaAffx.16207.1.S1_at | 0.475 | BE821188 | Cell Structure | | | Beta-glucosidase, lactase phlorizinhydrolase, and |
| | | | | | | | related proteins |
| 109 | GmaAffx.8712.1.S1_s_at | 0.474 | AW349107 | Disease & Defense | | | |
| 110 | Gma.1951.2.S1_a_at | 0.474 | CA783485 | Protein Destination & Storage | | | Serine carboxypeptidases |
| 111 | GmaAffx.46684.1.S1_at | 0.474 | BQ081034 | Signal Transduction | Protein | | |
| | | | | | Kinase | | |
| 112 | Gma.13000.1.S1_at | 0.474 | CD415111 | Unclassified - Hypothetical Protein | | | |
| | | | | Supported by cDNA | | | |
| 113 | Gma.9205.1.S1_at | 0.473 | BM528605 | Unclassified - Hypothetical Protein | | | |
| | | | | Supported by cDNA | | | |
| 114 | Gma.13035.1.S1_at | 0.473 | CD396536 | No Homology to Known Proteins | | | |
| 115 | Gma.285.1.S1_at | 0.472 | BG726992 | Cell Growth & Division | | | |
| 116 | Gma.10824.1.S1_at | 0.472 | BE824257 | Unclassified - Hypothetical Protein | | | Magnesium transporters: CorA family |
| | | | | Supported by cDNA | | | |
| 117 | GmaAffx.91641.1.S1_s_at | 0.472 | CF807366 | Disease & Defense | | | Acyl-CoA synthetase |
| 118 | Gma.4610.1.S1_s_at | 0.472 | BM522856 | No Homology to Known Proteins | | | |
| 119 | GmaAffx.86136.1.S1_at | 0.471 | AI440673 | Unclassified - Protein with | | | |
| | | | | Unknown Function | | | |
| 120 | Gma.6498.1.A1_at | 0.471 | BE824396 | Unclassified - Hypothetical Protein | | | |
| | | | | Supported by cDNA | | | |
| 121 | GmaAffx.92136.1.S1_s_at | 0.471 | CF807451 | Signal Transduction | Protein | | Mannosyltransferase |
| | | | | | Kinase | | |
| 122 | GmaAffx.26320.1.S1_at | 0.469 | BE440416 | Unclassified - Hypothetical Protein | | | |
| | | | | Supported by cDNA | | | |
| 123 | Gma.1332.1.S1_s_at | 0.468 | BI970666 | Secondary Metabolism | Ethylene | | 1-aminocyclopropane-1-carboxylate synthase, and related |
| | | | | | Biosynthesis | | proteins |
| 124 | Gma.16477.1.S1_at | 0.467 | BE824134 | No Homology to Known Proteins | | | |
| 125 | GmaAffx.4936.1.S1_at | 0.467 | AW666270 | Transporter | | | Multidrug/pheromone exporter, ABC |
| | | | | | | | superfamily |
| 126 | Gma.7824.1.S1_at | 0.465 | BU550492 | Metabolism | | | Kynurenine 3-monooxygenase and related |
| | | | | | | | flavoprotein monooxygenases |
| 127 | Gma.3283.1.S1_at | 0.465 | BQ298733 | No Homology to Known Proteins | | | |
| 128 | GmaAffx.89049.1.S1_s_at | 0.464 | CK606168 | Intracellular Traffic | | | Annexin |
| 129 | Gma.10026.1.S1_at | 0.464 | BU546301 | Unclassified - Protein with | | | |
| | | | | Unknown Function | | | |
| 130 | Gma.16896.1.A1_at | 0.462 | CD416188 | No Homology to Known Proteins | | | |
| 131 | Gma.1980.1.A1_at | 0.462 | BM520466 | Signal Transduction | Protein | AtCIPKs | Serine/threonine protein kinase |
| | | | | | Kinase | | |
| 132 | GmaAffx.49130.1.S1_at | 0.462 | BE555035 | No Homology to Known Proteins | | | |
| 133 | GmaAffx.14141.1.A1_at | 0.462 | BI973009 | No Homology to Known Proteins | | | |
| 134 | Gma.1528.1.S1_at | 0.461 | CD403623 | Transcription | Transcription | Basic Helix- | |
| | | | | | Factor | Loop-Helix | |
| | | | | | | (bHLH) | |
| 135 | GmaAffx.86392.1.S1_at | 0.46 | AI736754 | Signal Transduction | Calcium- | | |
| | | | | | Binding | | |
| 136 | Gma.10581.1.S1_at | 0.46 | AW396439 | Unclassified - Hypothetical Protein | | | Predicted haloacid dehalogenase-like hydrolase |
| | | | | Supported by cDNA | | | |
| 137 | GmaAffx.35729.1.S1_s_at | 0.46 | BE806047 | Unclassified - Hypothetical Protein | | | Predicted E3 ubiquitin ligase |
| | | | | Supported by cDNA | | | |
| 138 | GmaAffx.65419.1.S1_at | 0.459 | CD405955 | Signal Transduction | Protein | | |
| | | | | | Kinase | | |
| 139 | Gma.412.1.S1_at | 0.459 | BE607835 | Protein Destination & Storage | | | Ubiquitin-protein ligase |
| 140 | GmaAffx.90069.1.S1_s_at | 0.459 | CF805794 | Unclassified - Hypothetical Protein | | | Leucine rich repeat proteins, some proteins |
| | | | | Supported by cDNA | | | contain F-box |
| 141 | Gma.1839.2.S1_a_at | 0.459 | AW507949 | Metabolism | | | |
| 142 | Gma.17288.1.A1_at | 0.458 | AW310692 | No Homology to Known Proteins | | | |
| 143 | GmaAffx.58712.1.S1_at | 0.458 | BF325034 | Transporter | | | Transporter, ABC superfamily (Breast cancer |
| | | | | | | | resistance protein) |
| 144 | Gma.850.1.S1_at | 0.458 | BE822050 | Unclassified - Hypothetical Protein | | | |
| | | | | NOT Supported by cDNA | | | |
| 145 | GmaAffx.91731.1.S1_s_at | 0.458 | CF807456 | Secondary Metabolism | Ethylene | | Iron/ascorbate family oxidoreductases |
| | | | | | Biosynthesis | | |
| 146 | GmaAffx.74868.1.S1_at | 0.457 | CA820573 | No Homology to Known Proteins | | | |
| 147 | GmaAffx.29254.1.S1_at | 0.456 | AW760361 | Transcription | Transcription | Basic Helix- | |
| | | | | | Factor | Loop-Helix | |
| | | | | | | (bHLH) | |
| 148 | Gma.4394.1.S1_a_at | 0.456 | CD409273 | Transcription | Transcription | Homeodomain | Transcription factor HEX, contains HOX and |
| | | | | | Factor | | HALZ domains |
| 149 | Gma.17759.1.S1_at | 0.456 | BU927041 | Transcription | Transcription | AUX/1AA | |
| | | | | | Factor | | |
| 150 | Gma.10393.1.S1_at | 0.455 | BE804608 | Unclassified - Hypothetical Protein | | | Endoplasmic reticulum protein EP58, contains |
| | | | | NOT Supported by cDNA | | | filamin rod domain and KDEL motif |
| 151 | Gma.3591.1.S1_a_at | 0.455 | BE440266 | Secondary Metabolism | Ethylene | | Iron/ascorbate family oxidoreductases |
| | | | | | Biosynthesis | | |
| 152 | Gma.1573.2.S1_a_at | 0.454 | BI974098 | Metabolism | | | |
| 153 | GmaAffx.90789.1.S1_s_at | 0.454 | CF806514 | Unclassified - Protein with | | | |
| | | | | Unknown Function | | | |
| 154 | GmaAffx.67113.1.S1_at | 0.454 | BE329541 | No Homology to Known Proteins | | | |
| 155 | GmaAffx.51444.1.S1_at | 0.453 | BM178639 | Secondary Metabolism | | | Multicopper oxidases |
| 156 | Gma.1068.1.S1_at | 0.453 | L19360.1 | Signal Transduction | Protein | | Serine/threonine protein kinase |
| | | | | | Kinase | | |
| 157 | GmaAffx.44471.1.A1_at | 0.452 | CA851833 | Unclassified - Hypothetical Protein | | | |
| | | | | NOT Supported by cDNA | | | |
| 158 | GmaAffx.9145.1.S1_s_at | 0.452 | BU763814 | Disease & Defense | | | |
| 159 | Gma.3531.1.S1_s_at | 0.452 | BI498803 | Unclassified - Hypothetical Protein | | | FOG: PPR repeat |
| | | | | Supported by cDNA | | | |
| 160 | GmaAffx.84209.1.S1_at | 0.452 | BQ610588 | Signal Transduction | Histidine | | Sensory transduction histidine kinase |
| | | | | | Kinase | | |
| 161 | GmaAffx.494.1.S1_at | 0.452 | AI938330 | Cell Structure | | | Chitinase |
| 162 | Gma.16940.1.S1_at | 0.451 | AW310039 | Transcription | Transcription | Homeodomain | Transcription factor MEIS1 and related HOX |
| | | | | | Factor | | domain proteins |
| 163 | GmaAffx.32011.1.S1_at | 0.449 | BI701198 | No Homology to Known Proteins | | | |
| 164 | Gma.3963.2.S1_at | 0.449 | BE608192 | Unclassified - Hypothetical Protein | | | Zn-finger protein |
| | | | | NOT Supported by cDNA | | | |
| 165 | Gma.1263.1.S1_at | 0.449 | AY144180.1 | Metabolism | | | Ca2+/calmodulin-dependent protein kinase, EF- |
| | | | | | | | Hand protein superfamily |
| 166 | GmaAffx.72346.1.S1_at | 0.448 | BF424359 | Unclassified - Hypothetical Protein | | | Speckle-type POZ protein SPOP and related |
| | | | | NOT Supported by cDNA | | | proteins with TRAF, MATH and BTB/POZ |
| | | | | | | | domains |
| 167 | Gma.10144.2.S1_at | 0.447 | BU080819 | Metabolism | | | |
| 168 | GmaAffx.82263.1.S1_s_at | 0.447 | AW568454 | No Homology to Known Proteins | | | |
| 169 | Gma.12068.1.A1_at | 0.446 | BU547739 | Transporter | | | Transporter, ABC superfamily (Breast cancer |
| | | | | | | | resistance protein) |
| 170 | GmaAffx.83880.1.A1_at | 0.446 | BE822640 | Disease & Defense | | | |
| 171 | GmaAffx.9040.1.S1_at | 0.446 | AW101738 | Unclassified - Hypothetical Protein | | | |
| | | | | Supported by cDNA | | | |
| 172 | Gma.3893.3.S1_at | 0.446 | BG651854 | Unclassified - Hypothetical Protein | | | |
| | | | | Supported by cDNA | | | |
| 173 | GmaAffx.92184.1.S1_s_at | 0.445 | CF807909 | Unclassified - Protein with | | | |
| | | | | Unknown Function | | | |
| 174 | GmaAffx.45957.2.S1_at | 0.445 | BI974135 | No Homology to Known Proteins | | | |
| 175 | Gma.6264.1.S1_at | 0.444 | BQ612558 | Unclassified - Hypothetical Protein | | | |
| | | | | Supported by cDNA | | | |
| 176 | GmaAffx.86514.1.S1_at | 0.444 | BG790017 | Transcription | Transcription | ARF | |
| | | | | | Factor | | |
| 177 | GmaAffx.93182.1.S1_s_at | 0.443 | CF808907 | Secondary Metabolism | Ethylene | | Iron/ascorbate family oxidoreductases |
| | | | | | Biosynthesis | | |
| 178 | GmaAffx.58073.1.S1_at | 0.443 | CA852105 | Unclassified - Hypothetical Protein | | | |
| | | | | Supported by cDNA | | | |
| 179 | Gma.11028.1.S1_s_at | 0.443 | BE823084 | Metabolism | | | Glyoxylate/hydroxypyruvate reductase (D-isomer |
| | | | | | | | specific 2-hydroxy acid dehydrogenase |
| | | | | | | | superfamily) |
| 180 | Gma.6550.2.S1_s_at | 0.442 | BM086044 | Metabolism | | | Hydroxyindole-O-methyltransferase and related |
| | | | | | | | SAM-dependent methyltransferases |
| 181 | GmaAffx.91266.1.S1_s_at | 0.442 | CF806991 | Unclassified - Hypothetical Protein | | | |
| | | | | Supported by cDNA | | | |
| 182 | Gma.5331.1.S1_a_at | 0.442 | BI892702 | Transcription | Transcription | NAC | |
| | | | | | Factor | Domain | |
| 183 | GmaAffx.18880.1.S1_at | 0.442 | BM094235 | Signal Transduction | Protein | | Serine/threonine protein kinase |
| | | | | | Kinase | | |
| 184 | Gma.3579.1.S1_at | 0.442 | AW349799 | Unclassified - Hypothetical Protein | | | Kelch repeat-containing proteins |
| | | | | Supported by cDNA | | | |
| 185 | Gma.14471.4.S1_x_at | 0.441 | BQ081409 | Transporter | | | Aquaporin (major intrinsic protein family) |
| 186 | GmaAffx.65140.1.S1_at | 0.441 | BI700031 | No Homology to Known Proteins | | | |
| 187 | Gma.1895.1.A1_at | 0.44 | AW309584 | Protein Destination & Storage | | | ER lumen protein retaining receptor |
| 188 | Gma.16528.1.S1_at | 0.44 | CD398706 | Unclassified - Hypothetical Protein | | | |
| | | | | Supported by cDNA | | | |
| 189 | Gma.10950.1.S1_at | 0.44 | BI699798 | Unclassified - Hypothetical Protein | | | |
| | | | | Supported by cDNA | | | |
| 190 | Gma.15996.1.S1_at | 0.439 | BQ454193 | Unclassified - Hypothetical Protein | | | |
| | | | | Supported by cDNA | | | |
| 191 | GmaAffx.1785.1.S1_at | 0.439 | BF325331 | Transcription | Transcription | Homeodomain | Transcription factor HEX, contains HOX and |
| | | | | | Factor | | HALZ domains |
| 192 | GmaAffx.55953.1.S1_at | 0.439 | BQ629142 | Unclassified - Hypothetical Protein | | | FOG: Predicted E3 ubiquitin ligase |
| | | | | NOT Supported by cDNA | | | |
| 193 | GmaAffx.93553.1.S1_s_at | 0.439 | CF809278 | Metabolism | | | |
| 194 | GmaAffx.74194.1.S1_at | 0.438 | BI968058 | Metabolism | | | 17 beta-hydroxysteroid dehydrogenase type 3, |
| | | | | | | | HSD17B3 |
| 195 | GmaAffx.87793.2.S1_at | 0.437 | BQ740671 | Signal Transduction | Protein | | Glycogen synthase kinase-3 |
| | | | | | Kinase | | |
| 196 | GmaAffx.36470.1.A1_at | 0.436 | BU550552 | Unclassified - Hypothetical Protein | | | |
| | | | | Supported by cDNA | | | |
| 197 | GmaAffx.42856.1.S1_at | 0.435 | BM567981 | Transcription | Transcription | Basic Helix- | |
| | | | | | Factor | Loop-Helix | |
| | | | | | | (bHLH) | |
| 198 | GmaAffx.1301.78.S1_s_at | 0.435 | AW394497 | No Homology to Known Proteins | | | |
| 199 | GmaAffx.29951.2.S1_at | 0.434 | AW100899 | Metabolism | | | Dehydrogenase kinase |
| 200 | Gma.11334.1.S1_a_at | 0.433 | BI967327 | Cell Structure | | | O-methyltransferase |
| 201 | Gma.5559.1.S1_s_at | 0.433 | BM121524 | Unclassified - Hypothetical Protein | | | |
| | | | | Supported by cDNA | | | |
| 202 | GmaAffx.92230.1.A1_s_at | 0.432 | CF807955 | Disease & Defense | | | |
| 203 | GmaAffx.87679.1.S1_at | 0.431 | BM523734 | Metabolism | Trehalose | | Trehalose-6-phosphate synthase component |
| | | | | | Biosynthesis | | TPS1 and related subunits |
| 204 | Gma.398.1.A1_at | 0.43 | AW350679 | Metabolism | | | Homocysteine S-methyltransferase |
| 205 | Gma.4665.1.A1_at | 0.43 | AW309815 | No Homology to Known Proteins | | | |
| 206 | GmaAffx.27331.1.S1_at | 0.428 | BM520297 | Unclassified - Hypothetical Protein | | | |
| | | | | NOT Supported by cDNA | | | |
| 207 | GmaAffx.34975.1.S1_at | 0.428 | BU547817 | Unclassified - Hypothetical Protein | | | |
| | | | | Supported by cDNA | | | |
| 208 | Gma.10969.1.S1_a_at | 0.426 | U36191.1 | Metabolism | | | |
| 209 | Gma.1661.1.S1_at | 0.424 | BE821382 | Metabolism | | | |
| 210 | Gma.2080.2.S1_a_at | 0.424 | BM520916 | Secondary Metabolism | | | |
| 211 | GmaAffx.25612.1.S1_at | 0.424 | AW317577 | No Homology to Known Proteins | | | |
| 212 | GmaAffx.50677.1.S1_s_at | 0.423 | BI971744 | No Homology to Known Proteins | | | |
| 213 | GmaAffx.89624.1.A1_s_at | 0.422 | CK606100 | Transcription | Transcription | Basic | |
| | | | | | Factor | Leucine | |
| | | | | | | Zipper | |
| | | | | | | (bZIP) | |
| 214 | Gma.8514.1.S1_at | 0.422 | CA851666 | Protein Destination & Storage | | | Glutathione S-transferase |
| 215 | GmaAffx.91328.1.S1_at | 0.421 | CF807053 | Unclassified - Hypothetical Protein | | | Leucine rich repeat proteins, some proteins |
| | | | | Supported by cDNA | | | contain F-box |
| 216 | GmaAffx.47891.1.S1_s_at | 0.42 | BQ785880 | Cell Structure | | | |
| 217 | Gma.6278.1.S1_at | 0.419 | BQ612647 | Unclassified - Hypothetical Protein | | | |
| | | | | Supported by cDNA | | | |
| 218 | GmaAffx.487.1.S1_at | 0.419 | BG511495 | Transporter | | | Predicted transporter (major facilitator |
| | | | | | | | superfamily) |
| 219 | GmaAffx.84206.1.S1_at | 0.418 | BU760571 | Transcription | Transcription | NAC | |
| | | | | | Factor | Domain | |
| 220 | Gma.8058.1.A1_at | 0.417 | BM271479 | Signal Transduction | Protein | | Serine/threonine protein phosphatase |
| | | | | | Phosphatase | | |
| 221 | GmaAffx.47230.2.S1_at | 0.417 | BG652486 | Transporter | | | Amino acid transporters |
| 222 | Gma.17985.2.S1_at | 0.417 | BE802263 | Metabolism | | | |
| 223 | Gma.11277.2.S1_at | 0.416 | BI701238 | Unclassified - Hypothetical Protein | | | |
| | | | | NOT Supported by cDNA | | | |
| 224 | Gma.5400.1.S1_at | 0.416 | BM093775 | No Homology to Known Proteins | | | |
| 225 | GmaAffx.33121.1.S1_at | 0.416 | BQ611246 | Unclassified - Hypothetical Protein | | | |
| | | | | Supported by cDNA | | | |
| 226 | Gma.6550.2.S1_x_at | 0.413 | BM086044 | Metabolism | | | Hydroxyindole-O-methyltransferase and related |
| | | | | | | | SAM-dependent methyltransferases |
| 227 | GmaAffx.39703.1.S1_at | 0.413 | BE023656 | Protein Destination & Storage | | | Serine carboxypeptidases (lysosomal cathepsin |
| | | | | | | | A) |
| 228 | GmaAffx.15222.1.S1_at | 0.413 | BU550700 | Unclassified - Hypothetical Protein | | | |
| | | | | NOT Supported by cDNA | | | |
| 229 | GmaAffx.20146.1.S1_at | 0.413 | BE824274 | Unclassified - Protein with | | | Ca2+-binding protein (centrin/caltractin), EF- |
| | | | | Unknown Function | | | Hand superfamily protein |
| 230 | Gma.5586.2.S1_at | 0.412 | CD398384 | Transcription | Transcription | Unclassified | CCAAT-binding factor, subunit B (HAP2) |
| | | | | | Factor | | |
| 231 | GmaAffx.71016.1.S1_at | 0.411 | AW234455 | Unclassified - Hypothetical Protein | | | Arylacetamide deacetylase |
| | | | | Supported by cDNA | | | |
| 232 | Gma.10536.1.S1_at | 0.411 | BG508300 | No Homology to Known Proteins | | | |
| 233 | GmaAffx.74160.1.S1_at | 0.411 | BQ740845 | Protein Destination & Storage | | | |
| 234 | GmaAffx.91141.1.S1_at | 0.411 | CF806866 | Disease & Defense | | | |
| 235 | GmaAffx.11049.1.S1_at | 0.41 | BU545140 | Unclassified - Protein with | | | Phosphatidylethanolamine binding protein |
| | | | | Unknown Function | | | |
| 236 | Gma.17326.1.A1_at | 0.41 | AW568254 | Unclassified - Hypothetical Protein | | | Permease of the major facilitator superfamily |
| | | | | NOT Supported by cDNA | | | |
| 237 | Gma.16196.1.S1_at | 0.41 | AW350888 | Transcription | Transcription | Unclassified | |
| | | | | | Factor | | |
| 238 | Gma.6288.1.S1_at | 0.41 | AW348556 | Transporter | | | Aquaporin (major intrinsic protein family) |
| 239 | GmaAffx.23289.1.S1_at | 0.409 | CD392087 | No Homology to Known Proteins | | | |
| 240 | GmaAffx.93592.1.S1_x_at | 0.409 | CF809317 | Unclassified - Hypothetical Protein | | | |
| | | | | Supported by cDNA | | | |
| 241 | GmaAffx.92774.1.S1_at | 0.408 | CF808124 | Cell Structure | | | |
| 242 | GmaAffx.81851.1.S1_at | 0.408 | AW706751 | Unclassified - Hypothetical Protein | | | |
| | | | | Supported by cDNA | | | |
| 243 | GmaAffx.92927.1.S1_at | 0.407 | CF805992 | No Homology to Known Proteins | | | |
| 244 | GmaAffx.64794.1.S1_at | 0.407 | CA801749 | Unclassified - Hypothetical Protein | | | |
| | | | | Supported by cDNA | | | |
| 245 | GmaAffx.478.2.S1_at | 0.406 | BU763963 | Transcription | Transcription | Homeodomain | Transcription factor MEIS1 and related HOX |
| | | | | | Factor | | domain proteins |
| 246 | GmaAffx.24368.1.A1_at | 0.405 | BU545331 | No Homology to Known Proteins | | | |
| 247 | Gma.1394.2.S1_a_at | 0.404 | BI470370 | Metabolism | | | |
| 248 | GmaAffx.84424.1.S1_at | 0.404 | AW311543 | Metabolism | | | Alcohol dehydrogenase, class V |
| 249 | GmaAffx.93373.1.S1_s_at | 0.403 | CF809098 | Unclassified - Hypothetical Protein | | | |
| | | | | Supported by cDNA | | | |
| 250 | GmaAffx.34449.1.S1_at | 0.403 | AW164447 | No Homology to Known Proteins | | | |
| 251 | GmaAffx.64037.1.S1_at | 0.403 | BI321162 | Metabolism | Cytochrome | CYP82F | Cytochrome P450 CYP2 subfamily |
| | | | | | P450 | | |
| 252 | Gma.6709.1.S1_at | 0.402 | BE823291 | Signal Transduction | Protein | AtCIPKs | Serine/threonine protein kinase |
| | | | | | Kinase | | |
| 253 | Gma.17015.1.S1_at | 0.402 | AW733363 | No Homology to Known Proteins | | | |
| 254 | Gma.3963.1.S1_at | 0.402 | AW202007 | Unclassified - Hypothetical Protein | | | Zn-finger protein |
| | | | | NOT Supported by cDNA | | | |
| 255 | Gma.8113.1.A1_at | 0.401 | BU548342 | No Homology to Known Proteins | | | |
| 256 | GmaAffx.88675.1.A1_at | 0.4 | CD417704 | Unclassified - Hypothetical Protein | | | |
| | | | | NOT Supported by cDNA | | | |
| 257 | GmaAffx.5255.1.S1_at | 0.398 | BG362929 | Transcription | Transcription | AP2/EREBP | |
| | | | | | Factor | | |
| 258 | Gma.5294.1.S1_s_at | 0.398 | BE658252 | Unclassified - Hypothetical Protein | | | |
| | | | | Supported by cDNA | | | |
| 259 | Gma.3776.1.S1_at | 0.398 | AW780736 | Metabolism | | | Glutaredoxin and related proteins |
| 260 | GmaAffx.44396.1.S1_at | 0.398 | BE330360 | No Homology to Known Proteins | | | |
| 261 | GmaAffx.80393.1.A1_at | 0.397 | BE658680 | No Homology to Known Proteins | | | |
| 262 | GmaAffx.82936.1.S1_at | 0.397 | AI938364 | Unclassified - Protein with | | | |
| | | | | Unknown Function | | | |
| 263 | Gma.6075.1.S1_a_at | 0.396 | AW317183 | Disease & Defense | | | |
| 264 | GmaAffx.62364.1.S1_at | 0.395 | AW733764 | Protein Destination & Storage | | | Dipeptidyl aminopeptidase |
| 265 | GmaAffx.80900.1.S1_at | 0.393 | BE347494 | Metabolism | | | Iron/ascorbate family oxidoreductases |
| 266 | GmaAffx.79390.3.S1_at | 0.393 | BU082743 | Transcription | Transcription | Basic | FOG: Armadillo/beta-catenin-like repeats |
| | | | | | Factor | Leucine | |
| | | | | | | Zipper | |
| | | | | | | (bZIP) | |
| 267 | GmaAffx.4888.1.A1_at | 0.392 | BI967635 | No Homology to Known Proteins | | | |
| 268 | Gma.6727.1.S1_at | 0.39 | Y10493.1 | Metabolism | Cytochrome | CYP7IB | Cytochrome P450 CYP2 subfamily |
| | | | | | P450 | | |
| 269 | GmaAffx.79390.1.S1_at | 0.39 | BU550458 | Transcription | Transcription | Basic | FOG: Armadillo/beta-catenin-like repeats |
| | | | | | Factor | Leucine | |
| | | | | | | Zipper | |
| | | | | | | (bZIP) | |
| 270 | GmaAffx.47590.1.S1_at | 0.39 | CA853861 | Metabolism | | | UDP-glucuronosyl and UDP-glucosyl transferase |
| 271 | GmaAffx.99.4.S1_at | 0.39 | BQ742171 | Unclassified - Protein with | | | Microtubule-binding protein (translationally |
| | | | | Unknown Function | | | controlled tumor protein) |
| 272 | Gma.4822.1.S1_at | 0.388 | BI969249 | Disease & Defense | | | |
| 273 | GmaAffx.63822.1.S1_at | 0.388 | BM520838 | Disease & Defense | | | Purple (tartrate-resistant) acid phosphatase |
| 274 | GmaAffx.87547.2.S1_at | 0.388 | BQ080473 | Cell Structure | | | Beta-glucosidase, lactase phlorizinhydrolase, and |
| | | | | | | | related proteins |
| 275 | GmaAffx.78297.1.S1_at | 0.387 | BM731185 | Unclassified - Hypothetical Protein | | | FOG: Predicted E3 ubiquitin ligase |
| | | | | Supported by cDNA | | | |
| 276 | Gma.10827.2.S1_s_at | 0.386 | BQ612452 | Protein Destination & Storage | Storage | | |
| | | | | | Protein | | |
| 277 | GmaAffx.82000.1.S1_at | 0.385 | BI094895 | Unclassified - Hypothetical Protein | | | |
| | | | | Supported by cDNA | | | |
| 278 | Gma.13800.1.A1_at | 0.384 | CD405906 | Unclassified - Hypothetical Protein | | | |
| | | | | NOT Supported by cDNA | | | |
| 279 | GmaAffx.24451.1.A1_at | 0.384 | BU550405 | Metabolism | Cytochrome | CYP90C | Cytochrome P450 CYP4/CYP19/CYP26 |
| | | | | | P450 | | subfamilies |
| 280 | GmaAffx.85657.1.S1_s_at | 0.384 | AW185911 | Unclassified - Protein with | | | FOG: Kelch repeat |
| | | | | Unknown Function | | | |
| 281 | Gma.13736.1.A1_at | 0.384 | CD404408 | Secondary Metabolism | | | Multicopper oxidases |
| 282 | GmaAffx.84338.1.S1_at | 0.384 | AI856789 | Unclassified - Hypothetical Protein | | | Predicted E3 ubiquitin ligase |
| | | | | Supported by cDNA | | | |
| 283 | GmaAffx.53108.1.S1_at | 0.384 | BM093748 | Signal Transduction | Protein | | Serine/threonine protein kinase |
| | | | | | Kinase | | |
| 284 | Gma.16875.1.A1_at | 0.383 | CD406723 | Metabolism | | | Aldo/keto reductase family proteins |
| 285 | GmaAffx.44484.1.S1_at | 0.383 | BE475213 | Unclassified - Hypothetical Protein | | | |
| | | | | NOT Supported by cDNA | | | |
| 286 | Gma.17070.1.S1_at | 0.382 | BE822463 | Disease & Defense | | | O-methyltransferase |
| 287 | Gma.2376.1.S1_at | 0.382 | CA852095 | Transcription | Transcription | Homeodomain | Transcription factor MEIS1 and related HOX |
| | | | | | Factor | | domain proteins |
| 288 | Gma.1642.3.S1_s_at | 0.382 | BM891895 | Unclassified - Hypothetical Protein | | | |
| | | | | Supported by cDNA | | | |
| 289 | Gma.16842.1.A1_at | 0.382 | CD392652 | No Homology to Known Proteins | | | |
| 290 | Gma.2686.1.S1_s_at | 0.381 | BI970455 | Secondary Metabolism | | | |
| 291 | GmaAffx.90570.1.S1_at | 0.381 | CF806295 | Unclassified - Hypothetical Protein | | | |
| | | | | Supported by cDNA | | | |
| 292 | GmaAffx.83377.2.S1_at | 0.38 | BM893101 | Transcription | Transcription | Basic Helix- | |
| | | | | | Factor | Loop-Helix | |
| | | | | | | (bHLH) | |
| 293 | Gma.7494.1.S1_at | 0.38 | BU548105 | Signal Transduction | Protein | | Serine/threonine protein phosphatase |
| | | | | | Phosphatase | | |
| 294 | GmaAffx.1301.68.S1_s_at | 0.378 | BI974374 | Intracellular Traffic | | | Annexin |
| 295 | GmaAffx.15983.2.S1_s_at | 0.377 | AW279540 | Signal Transduction | Protein | | Serine/threonine protein phosphatase |
| | | | | | Phosphatase | | |
| 296 | GmaAffx.88786.1.A1_s_at | 0.376 | CK606552 | Cell Structure | | | O-methyltransferase |
| 297 | Gma.5478.1.S1_at | 0.374 | AW350606 | Metabolism | | | |
| 298 | Gma.6550.3.S1_s_at | 0.374 | AW234450 | Metabolism | | | Hydroxyindole-O-methyltransferase and related |
| | | | | | | | SAM-dependent methyltransferases |
| 299 | Gma.1490.1.S1_at | 0.374 | AB000097.1 | Cell Structure | | | Chitinase |
| 300 | GmaAffx.11999.1.S1_at | 0.374 | AW567661 | No Homology to Known Proteins | | | |
| 301 | GmaAffx.41990.1.S1_at | 0.374 | CA936708 | No Homology to Known Proteins | | | |
| 302 | GmaAffx.66451.1.S1_at | 0.373 | AW309198 | Secondary Metabolism | | | |
| 303 | GmaAffx.79888.1.S1_at | 0.372 | AI988123 | Signal Transduction | Protein | | |
| | | | | | Kinase | | |
| 304 | Gma.3420.1.S1_at | 0.37 | CD406082 | Unclassified - Hypothetical Protein | | | |
| | | | | Supported by cDNA | | | |
| 305 | GmaAffx.80098.1.S1_at | 0.37 | BG042861 | Metabolism | | | Arylacetamide deacetylase |
| 306 | GmaAffx.1128.1.S1_at | 0.37 | AW348608 | Unclassified - Protein with | | | Copper chaperone |
| | | | | Unknown Function | | | |
| 307 | Gma.12094.1.A1_at | 0.37 | CD395088 | Unclassified - Hypothetical Protein | | | |
| | | | | Supported by cDNA | | | |
| 308 | GmaAffx.90812.1.S1_s_at | 0.37 | CF806537 | Disease & Defense | | | |
| 309 | GmaAffx.9926.2.S1_at | 0.368 | BF597587 | Unclassified - Hypothetical Protein | | | |
| | | | | Supported by cDNA | | | |
| 310 | Gma.6640.1.S1_at | 0.367 | BQ628278 | Disease & Defense | | | |
| 311 | GmaAffx.1301.59.S1_at | 0.367 | BI974591 | Intracellular Traffic | | | Annexin |
| 312 | GmaAffx.21041.1.A1_at | 0.367 | AW350819 | No Homology to Known Proteins | | | |
| 313 | Gma.16359.1.A1_at | 0.366 | BI970359 | Metabolism | | | GP1-alpha-mannosyltransferase III (GP110/PIG- |
| | | | | | | | B) involved in glycosylphosphatidylinositol |
| | | | | | | | anchor biosynthesis |
| 314 | Gma.1195.1.S1_s_at | 0.366 | AI442801 | Metabolism | | | Predicted flavoprotein |
| 315 | GmaAffx.91205.1.S1_at | 0.365 | CF807869 | Metabolism | | | Glyoxylate/hydroxypyruvate reductase (D-isomer |
| | | | | | | | specific 2-hydroxy acid dehydrogenase |
| | | | | | | | superfamily) |
| 316 | GmaAffx.93342.1.S1_s_at | 0.364 | CF809067 | Disease & Defense | | | Glutathione peroxidase |
| 317 | GmaAffx.74899.1.S1_at | 0.364 | CA852377 | No Homology to Known Proteins | | | |
| 318 | GmaAffx.15613.1.S1_at | 0.364 | AI939073 | Unclassified - Hypothetical Protein | | | |
| | | | | NOT Supported by cDNA | | | |
| 319 | GmaAffx.18953.1.S1_at | 0.363 | BM178798 | No Homology to Known Proteins | | | |
| 320 | GmaAffx.46837.1.S1_at | 0.362 | BU550919 | Secondary Metabolism | | | Multicopper oxidases |
| 321 | GmaAffx.50646.1.A1_at | 0.362 | AW310451 | Unclassified - Hypothetical Protein | | | Molybdenum cofactor sulfurase |
| | | | | Supported by cDNA | | | |
| 322 | Gma.2887.2.S1_x_at | 0.362 | BI701520 | Secondary Metabolism | | | Phenylalanine and histidine ammonia-lyase |
| 323 | Gma.17835.1.S1_at | 0.361 | BE822840 | Metabolism | | | |
| 324 | Gma.6719.2.S1_at | 0.361 | BG726773 | No Homology to Known Proteins | | | |
| 325 | GmaAffx.86927.1.S1_at | 0.36 | CA937162 | No Homology to Known Proteins | | | |
| 326 | Gma.3804.1.A1_at | 0.36 | AW310249 | Disease & Defense | | | |
| 327 | GmaAffx.93189.1.S1_s_at | 0.359 | CF808914 | Metabolism | | | Carbon-nitrogen hydrolase |
| 328 | GmaAffx.50980.1.S1_s_at | 0.358 | BE823095 | Signal Transduction | Protein | | |
| | | | | | Phosphatase | | |
| 329 | Gma.821.1.S1_at | 0.357 | AF243371.1 | Metabolism | | | Glutathione S-transferase |
| 330 | GmaAffx.18599.1.S1_at | 0.357 | BI787464 | Secondary Metabolism | Amines | | Amine oxidase |
| | | | | | Biosynthesis | | |
| 331 | Gma.5634.1.S1_at | 0.356 | BE806891 | Secondary Metabolism | | | Steroid reductase |
| 332 | GmaAffx.74256.1.S1_at | 0.356 | BE057293 | No Homology to Known Proteins | | | |
| 333 | Gma.2086.1.S1_at | 0.355 | AF035671.1 | Metabolism | Amino Acid | | Arginase family protein |
| | | | | | Biosynthesis/ | | |
| | | | | | Metabolism | | |
| 334 | Gma.73.1.S1_at | 0.354 | AF098662.1 | Protein Destination & Storage | | | |
| 335 | GmaAffx.58125.1.A1_at | 0.353 | BE347561 | Transcription | Transcription | Basic Helix- | |
| | | | | | Factor | Loop-Helix | |
| | | | | | | (bHLH) | |
| 336 | Gma.12731.2.A1_s_at | 0.353 | CD395848 | Unclassified - Hypothetical Protein | | | Aquaporin (major intrinsic protein family) |
| | | | | NOT Supported by cDNA | | | |
| 337 | GmaAffx.91960.1.S1_at | 0.353 | CF807685 | Metabolism | | | Branched chain alpha-keto acid dehydrogenase |
| | | | | | | | complex, alpha subunit |
| 338 | GmaAffx.87793.1.S1_at | 0.353 | BI941782 | Unclassified - Hypothetical Protein | | | |
| | | | | NOT Supported by cDNA | | | |
| 339 | GmaAffx.6790.1.S1_at | 0.352 | AI856683 | Disease & Defense | | | Soluble epoxide hydrolase |
| 340 | GmaAffx.15471.1.S1_at | 0.352 | BQ611747 | Transcription | Transcription | MYB | Transcription factor, Myb superfamily |
| | | | | | Factor | | |
| 341 | GmaAffx.92192.1.S1_s_at | 0.352 | CF806406 | Disease & Defense | | | |
| 342 | Gma.1929.1.S1_at | 0.352 | BU083650 | Transcription | Transcription | Homeodomain | Transcription factor HEX, contains HOX and |
| | | | | | Factor | | HALZ domains |
| 343 | Gma.17739.1.S1_at | 0.352 | AW310535 | Unclassified - Hypothetical Protein | | | |
| | | | | Supported by cDNA | | | |
| 344 | GmaAffx.59014.1.S1_s_at | 0.35 | AW733977 | No Homology to Known Proteins | | | |
| 345 | Gma.7120.2.S1_a_at | 0.35 | BG041285 | Metabolism | | | Alanine-glyoxylate aminotransferase AGT2 |
| 346 | GmaAffx.478.1.S1_at | 0.348 | BE822248 | Transcription | Transcription | Homeodomain | Transcription factor MEIS1 and related HOX |
| | | | | | Factor | | domain proteins |
| 347 | GmaAffx.11892.1.S1_at | 0.348 | BE820605 | Secondary Metabolism | | | Iron/ascorbate family oxidoreductases |
| 348 | Gma.3888.2.S1_at | 0.347 | BM139532 | Unclassified - Hypothetical Protein | | | Aldehyde reductase |
| | | | | NOT Supported by cDNA | | | |
| 349 | GmaAffx.82218.1.S1_at | 0.347 | BI702052 | Secondary Metabolism | | | Iron/ascorbate family oxidoreductases |
| 350 | GmaAffx.93642.1.S1_s_at | 0.347 | CF809367 | Disease & Defense | | | |
| 351 | Gma.6047.1.S1_s_at | 0.346 | BQ611159 | Unclassified - Protein with | | | |
| | | | | Unknown Function | | | |
| 352 | Gma.5634.1.S1_s_at | 0.344 | BE806891 | Secondary Metabolism | | | Steroid reductase |
| 353 | Gma.10969.3.S1_x_at | 0.343 | CD396552 | Metabolism | | | |
| 354 | Gma.18006.1.S1_at | 0.343 | CF922923 | Transcription | Transcription | Homeodomain | Transcription factor MEIS1 and related HOX |
| | | | | | Factor | | domain proteins |
| 355 | GmaAffx.62195.1.S1_at | 0.343 | AW472439 | No Homology to Known Proteins | | | |
| 356 | Gma.10854.1.S1_at | 0.343 | CA802334 | No Homology to Known Proteins | | | |
| 357 | GmaAffx.30593.1.S1_at | 0.342 | BE803365 | Unclassified - Hypothetical Protein | | | |
| | | | | Supported by cDNA | | | |
| 358 | Gma.5258.1.S1_at | 0.341 | BE658899 | No Homology to Known Proteins | | | |
| 359 | Gma.4401.2.S1_a_at | 0.339 | BI970725 | Disease & Defense | | | |
| 360 | GmaAffx.75740.1.S1_at | 0.338 | BG157373 | Unclassified - Hypothetical Protein | | | |
| | | | | NOT Supported by cDNA | | | |
| 361 | GmaAffx.64261.1.A1_at | 0.338 | BI969080 | Unclassified - Protein with | | | 1,4-benzoquinone reductase-like; Trp repressor |
| | | | | Unknown Function | | | binding protein-like/protoplast-secreted protein |
| 362 | Gma.4981.1.A1_at | 0.337 | BI970256 | No Homology to Known Proteins | | | |
| 363 | Gma.3522.1.S1_at | 0.336 | BM140209 | Unclassified - Hypothetical Protein | | | |
| | | | | Supported by cDNA | | | |
| 364 | GmaAffx.30792.1.A1_at | 0.335 | BU544815 | Unclassified - Hypothetical Protein | | | |
| | | | | Supported by cDNA | | | |
| 365 | Gma.7437.1.S1_at | 0.332 | BU547705 | Unclassified - Hypothetical Protein | | | |
| | | | | Supported by cDNA | | | |
| 366 | Gma.7254.1.S1_at | 0.332 | BU546166 | Unclassified - Hypothetical Protein | | | |
| | | | | Supported by cDNA | | | |
| 367 | GmaAffx.91312.1.S1_s_at | 0.332 | CF806002 | Transcription | Transcription | Homeodomain | Transcription factor HEX, contains HOX and |
| | | | | | Factor | | HALZ domains |
| 368 | Gma.14075.1.S1_at | 0.331 | CD415333 | Protein Destination & Storage | Storage | | Ca2+-independent phospholipase A2 |
| | | | | | Protein | | |
| 369 | Gma.15155.2.S1_at | 0.33 | BG789619 | Unclassified - Hypothetical Protein | | | |
| | | | | Supported by cDNA | | | |
| 370 | Gma.1622.1.A1_s_at | 0.33 | BU090523 | Signal Transduction | Protein | | FOG: Transposon-encoded proteins with TYA, |
| | | | | | Kinase | | reverse transcriptase, integrase domains in |
| | | | | | | | various combinations |
| 371 | GmaAffx.61872.1.S1_at | 0.329 | AW102386 | Signal Transduction | Protein | | |
| | | | | | Kinase | | |
| 372 | GmaAffx.50673.2.S1_s_at | 0.329 | BI787147 | Transcription | Transcription | NAC | |
| | | | | | Factor | Domain | |
| 373 | GmaAffx.26868.1.S1_at | 0.328 | BF325443 | Unclassified - Hypothetical Protein | | | Tomosyn and related SNARE-interacting |
| | | | | NOT Supported by cDNA | | | proteins |
| 374 | GmaAffx.91185.1.S1_at | 0.327 | CF806910 | Cell Structure | | | Pectin acetylesterase and similar proteins |
| 375 | GmaAffx.47230.1.S1_at | 0.327 | CA820051 | Transporter | | | Amino acid transporters |
| 376 | Gma.5622.1.S1_at | 0.326 | BG363493 | No Homology to Known Proteins | | | |
| 377 | Gma.5513.1.S1_x_at | 0.325 | AW310453 | Metabolism | | | Haloacid dehalogenase-like hydrolase |
| 378 | GmaAffx.26859.1.S1_s_at | 0.325 | BM085620 | Disease & Defense | | | Glutathione S-transferase |
| 379 | Gma.8490.1.S1_at | 0.323 | AB030492.1 | Metabolism | | | Protein involved in thiamine biosynthesis and |
| | | | | | | | DNA damage tolerance |
| 380 | Gma.10342.1.S1_at | 0.323 | BE058741 | Unclassified - Hypothetical Protein | | | |
| | | | | Supported by cDNA | | | |
| 381 | Gma.15605.1.S1_at | 0.321 | BM143798 | Transcription | Transcription | AP2/EREBP | |
| | | | | | Factor | | |
| 382 | GmaAffx.19118.1.S1_at | 0.32 | BM523731 | Unclassified - Hypothetical Protein | | | |
| | | | | NOT Supported by cDNA | | | |
| 383 | GmaAffx.56301.1.S1_at | 0.32 | BQ610805 | No Homology to Known Proteins | | | |
| 384 | Gma.6046.1.S1_at | 0.32 | BE022758 | Unclassified - Hypothetical Protein | | | |
| | | | | NOT Supported by cDNA | | | |
| 385 | Gma.6442.2.S1_at | 0.32 | BM270161 | Metabolism | | | Lysophospholipase |
| 386 | GmaAffx.92166.1.S1_s_at | 0.318 | CF806313 | Signal Transduction | Unclassified | | Aspartyl protease |
| 387 | GmaAffx.3711.1.A1_at | 0.318 | BE822116 | Unclassified - Hypothetical Protein | | | |
| | | | | NOT Supported by cDNA | | | |
| 388 | Gma.3745.1.S1_at | 0.318 | AW156339 | Secondary Metabolism | Amines | | Amine oxidase |
| | | | | | Biosynthesis | | |
| 389 | Gma.17798.2.S1_at | 0.317 | CD412748 | No Homology to Known Proteins | | | |
| 390 | GmaAffx.21924.1.S1_at | 0.316 | BQ611867 | Disease & Defense | | | |
| 391 | GmaAffx.37615.1.S1_at | 0.315 | BQ454150 | No Homology to Known Proteins | | | |
| 392 | Gma.1394.1.S1_at | 0.315 | BG839300 | Metabolism | | | |
| 393 | GmaAffx.89772.10.A1_s_at | 0.315 | CK606097 | Disease & Defense | | | |
| 394 | Gma.5215.1.S1_at | 0.313 | AI988141 | Unclassified - Hypothetical Protein | | | BCL2-associated athanogene-like proteins and |
| | | | | Supported by cDNA | | | related BAG family chaperone regulators |
| 395 | Gma.6623.1.S1_at | 0.313 | BI702268 | Unclassified - Protein with | | | 1.4-benzoquinone reductase-like; Trp repressor |
| | | | | Unknown Function | | | binding protein-like/protoplast-secreted protein |
| 396 | GmaAffx.74573.1.S1_at | 0.311 | BU763133 | No Homology to Known Proteins | | | |
| 397 | GmaAffx.21978.1.A1_at | 0.311 | CA803009 | No Homology to Known Proteins | | | |
| 398 | GmaAffx.76346.1.A1_at | 0.311 | BQ612378 | Energy | | | 1,4-benzoquinone reductase-like; Trp repressor |
| | | | | | | | binding protein-like/protoplast-secreted protein |
| 399 | Gma.1137.1.S1_x_at | 0.31 | BF067710 | Metabolism | | | |
| 400 | GmaAffx.81362.1.S1_at | 0.309 | BF324988 | Cell Structure | | | |
| 401 | GmaAffx.85528.1.S1_at | 0.309 | BE608070 | Unclassified - Hypothetical Protein | | | FOG: PPR repeat |
| | | | | Supported by cDNA | | | |
| 402 | GmaAffx.55124.1.S1_at | 0.309 | BI970821 | Unclassified - Hypothetical Protein | | | Metacaspase involved in regulation of apoptosis |
| | | | | Supported by cDNA | | | |
| 403 | GmaAffx.77269.1.S1_at | 0.308 | BM093698 | No Homology to Known Proteins | | | |
| 404 | Gma.15568.1.S1_at | 0.305 | AI442650 | Disease & Defense | | | |
| 405 | Gma.17686.1.S1_at | 0.304 | BF597378 | Metabolism | Cytochrome | CYP76C | Cytochrome P450 CYP2 subfamily |
| | | | | | P450 | | |
| 406 | Gma.15469.1.S1_at | 0.302 | BU546655 | Cell Structure | | | Alpha tubulin |
| 407 | Gma.15155.2.S1_a_at | 0.302 | BG789619 | Unclassified - Hypothetical Protein | | | |
| | | | | Supported by cDNA | | | |
| 408 | GmaAffx.57748.1.S1_at | 0.302 | AW306274 | No Homology to Known Proteins | | | |
| 409 | Gma.3976.1.S1_at | 0.301 | BI972268 | No Homology to Known Proteins | | | |
| 410 | Gma.15155.1.S1_a_at | 0.301 | BF423817 | Unclassified - Hypothetical Protein | | | |
| | | | | Supported by cDNA | | | |
| 411 | GmaAffx.61130.1.S1_at | 0.3 | CA799668 | Transporter | | | H+/oligopeptide symporter |
| 412 | GmaAffx.41078.1.S1_at | 0.3 | BI317149 | Metabolism | | | Alcohol dehydrogenase, class III |
| 413 | Gma.4093.2.S1_at | 0.3 | BQ081460 | Transporter | | | |
| 414 | GmaAffx.62509.2.S1_at | 0.299 | BE021475 | Transcription | Transcription | MYB | Transcription factor, Myb superfamily |
| | | | | | Factor | | |
| 415 | GmaAffx.37055.1.A1_at | 0.299 | BM142939 | Metabolism | | | UDP-glucuronosyl and UDP-glucosyl transferase |
| 416 | GmaAffx.68724.1.A1_at | 0.299 | BI970438 | No Homology to Known Proteins | | | |
| 417 | Gma.5867.1.A1_s_at | 0.297 | BI316163 | Metabolism | | | |
| 418 | Gma.752.1.A1_at | 0.297 | AW432463 | Transcription | Transcription | Basic Helix- | |
| | | | | | Factor | Loop-Helix | |
| | | | | | | (bHLH) | |
| 419 | GmaAffx.9926.1.A1_at | 0.296 | CD411291 | No Homology to Known Proteins | | | |
| 420 | Gma.4249.1.S1_at | 0.296 | BF324893 | Secondary Metabolism | | | Methyltransferase |
| 421 | GmaAffx.78163.1.S1_s_at | 0.296 | BM520857 | Signal Transduction | Protein | | Serine/threonine protein phosphatase |
| | | | | | Phosphatase | | |
| 422 | Gma.968.1.S1_at | 0.296 | D28876.1 | Protein Destination & Storage | | | Asparaginyl peptidases |
| 423 | GmaAffx.38555.2.S1_at | 0.296 | BI425873 | No Homology to Known Proteins | | | |
| 424 | Gma.7454.1.S1_a_at | 0.296 | CD405901 | Transcription | Transcription | AUX/1AA | |
| | | | | | Factor | | |
| 425 | GmaAffx.93328.1.S1_s_at | 0.295 | CF809053 | Secondary Metabolism | | | |
| 426 | Gma.2475.1.S1_at | 0.295 | BM522941 | Cell Structure | | | |
| 427 | Gma.7120.1.S1_at | 0.293 | BM093481 | Metabolism | | | Alanine-glyoxylate aminotransferase AGT2 |
| 428 | Gma.1716.1.S1_at | 0.292 | AW156801 | Unclassified - Hypothetical Protein | | | |
| | | | | Supported by cDNA | | | |
| 429 | Gma.14338.1.A1_at | 0.29 | CF923111 | Protein Destination & Storage | Storage | | |
| | | | | | Protein | | |
| 430 | GmaAffx.4968.1.S1_at | 0.289 | BI967691 | Unclassified - Hypothetical Protein | | | |
| | | | | Supported by cDNA | | | |
| 431 | Gma.10559.1.S1_s_at | 0.289 | BU544359 | No Homology to Known Proteins | | | |
| 432 | GmaAffx.16986.2.S1_at | 0.287 | AW277368 | Metabolism | | | Hydroxyindole-O-methyltransferase and related |
| | | | | | | | SAM-dependent methyltransferases |
| 433 | GmaAffx.24102.1.S1_at | 0.286 | BQ453911 | Unclassified - Hypothetical Protein | | | |
| | | | | Supported by cDNA | | | |
| 434 | GmaAffx.70005.1.S1_s_at | 0.284 | CA936335 | Secondary Metabolism | | | Iron/ascorbate family oxidoreductases |
| 435 | Gma.17921.2.S1_s_at | 0.284 | BM085020 | Metabolism | Sucrose | | Glycosyltransferase |
| | | | | | Biosynthesis | | |
| 436 | Gma.14471.2.S1_at | 0.284 | CD408292 | Transporter | | | Aquaporin (major intrinsic protein family) |
| 437 | Gma.5863.2.S1_s_at | 0.283 | AW350762 | Secondary Metabolism | | | |
| 438 | GmaAffx.56715.1.S1_at | 0.283 | BG041616 | Disease & Defense | | | |
| 439 | Gma.364.1.A1_at | 0.283 | AW309875 | Unclassified - Hypothetical Protein | | | |
| | | | | Supported by cDNA | | | |
| 440 | GmaAffx.90970.1.S1_s_at | 0.28 | CF806695 | Unclassified - Hypothetical Protein | | | |
| | | | | Supported by cDNA | | | |
| 441 | GmaAffx.13912.1.S1_at | 0.28 | BI699258 | Disease & Defense | | | Purple (tartrate-resistant) acid phosphatase |
| 442 | Gma.5513.1.S1_at | 0.277 | AW310453 | Metabolism | | | Haloacid dehalogenase-like hydrolase |
| 443 | GmaAffx.52826.3.S1_at | 0.276 | BF596503 | Signal Transduction | Protein | | Serine/threonine protein phosphatase |
| | | | | | Phosphatase | | |
| 444 | GmaAffx.71308.2.A1_at | 0.276 | AW569888 | No Homology to Known Proteins | | | |
| 445 | Gma.12684.1.S1_at | 0.275 | CD394773 | Transcription | Transcription | Zinc Finger | |
| | | | | | Factor | | |
| 446 | GmaAffx.93220.1.S1_at | 0.275 | CF808945 | No Homology to Known Proteins | | | |
| 447 | Gma.13150.1.A1_at | 0.274 | CD411577 | Unclassified - Protein with | Hormone | | |
| | | | | Unknown Function | Induced | | |
| 448 | GmaAffx.57469.1.S1_at | 0.274 | AW351054 | Unclassified - Hypothetical Protein | | | |
| | | | | Supported by cDNA | | | |
| 449 | Gma.2842.1.S1_at | 0.274 | BG511652 | Unclassified - Protein with | | | Predicted Yippee-type zinc-binding protein |
| | | | | Unknown Function | | | |
| 450 | GmaAffx.86745.1.S1_s_at | 0.271 | BI424960 | Disease & Defense | | | |
| 451 | Gma.6599.1.S1_at | 0.271 | BQ629084 | Cell Structure | | | |
| 452 | GmaAffx.75697.1.S1_s_at | 0.27 | BU090518 | Unclassified - Hypothetical Protein | | | |
| | | | | Supported by cDNA | | | |
| 453 | Gma.6033.1.S1_at | 0.27 | BI967767 | Unclassified - Hypothetical Protein | | | |
| | | | | Supported by cDNA | | | |
| 454 | Gma.4837.1.S1_at | 0.269 | BI969343 | Transporter | | | H+/oligopeptide symporter |
| 455 | Gma.382.1.A1_at | 0.268 | AW349068 | Metabolism | | | Arylacetamide deacetylase |
| 456 | GmaAffx.91555.1.A1_s_at | 0.268 | CF807280 | Metabolism | | | |
| 457 | GmaAffx.9911.1.S1_at | 0.267 | BU547808 | Unclassified - Hypothetical Protein | | | Tomosyn and related SNARE-interacting |
| | | | | NOT Supported by cDNA | | | proteins |
| 458 | Gma.3604.1.S1_at | 0.267 | AW349604 | Cell Structure | | | O-methyltransferase |
| 459 | Gma.17840.1.S1_at | 0.267 | BE821995 | No Homology to Known Proteins | | | |
| 460 | Gma.9086.1.S1_at | 0.263 | AW184932 | Cell Growth & Division | | | |
| 461 | GmaAffx.71842.1.S1_at | 0.263 | BE555317 | Metabolism | | | Possible pfkB family carbohydrate kinase |
| 462 | GmaAffx.86247.1.S1_s_at | 0.262 | BG238674 | Unclassified - Hypothetical Protein | | | |
| | | | | Supported by cDNA | | | |
| 463 | GmaAffx.62509.1.S1_s_at | 0.261 | BE022332 | Transcription | Transcription | MYB | Transcription factor, Myb superfamily |
| | | | | | Factor | | |
| 464 | GmaAffx.90104.1.S1_s_at | 0.26 | CF805829 | Transporter | | | Permease of the major facilitator superfamily |
| 465 | Gma.4874.1.A1_at | 0.259 | AI938293 | No Homology to Known Proteins | | | |
| 466 | Gma.2414.1.S1_at | 0.259 | BU551192 | Protein Destination & Storage | | | |
| 467 | Gma.3914.1.S1_at | 0.258 | BI702206 | Unclassified - Hypothetical Protein | | | |
| | | | | Supported by cDNA | | | |
| 468 | GmaAffx.84751.1.A1_at | 0.257 | AW349702 | Transcription | Transcription | MYB | Transcription factor, Myb superfamily |
| | | | | | Factor | | |
| 469 | GmaAffx.11172.1.A1_at | 0.256 | CD404556 | No Homology to Known Proteins | | | |
| 470 | GmaAffx.93591.1.S1_s_at | 0.256 | CF809316 | Metabolism | | | |
| 471 | Gma.6343.1.S1_at | 0.25 | AI442963 | Unclassified - Hypothetical Protein | | | |
| | | | | NOT Supported by cDNA | | | |
| 472 | Gma.6999.2.S1_s_at | 0.249 | X60043.1 | Disease & Defense | | | |
| 473 | Gma.4222.1.S1_at | 0.248 | BU083971 | Unclassified - Hypothetical Protein | | | Arylacetamide deacetylase |
| | | | | NOT Supported by cDNA | | | |
| 474 | GmaAffx.51409.1.S1_at | 0.247 | BM309035 | Transporter | | | Synaptic vesicle transporter SVOP and related |
| | | | | | | | transporters (major facilitator superfamily) |
| 475 | Gma.3987.1.S1_at | 0.247 | BQ453408 | Unclassified - Hypothetical Protein | | | Sodium sulfate symporter and related arsenite |
| | | | | NOT Supported by cDNA | | | permeases |
| 476 | Gma.9828.1.S1_at | 0.245 | AF529301.1 | Metabolism | | | Haloacid dehalogenase-like hydrolase |
| 477 | GmaAffx.89259.1.S1_s_at | 0.245 | CK605646 | Metabolism | | | |
| 478 | GmaAffx.70008.1.S1_at | 0.244 | BU762337 | Transcription | Transcription | MYB | Transcription factor, Myb superfamily |
| | | | | | Factor | | |
| 479 | Gma.6679.1.A1_at | 0.241 | BU544081 | Transcription | Transcription | MYB | Transcription factor, Myb superfamily |
| | | | | | Factor | | |
| 480 | Gma.7422.1.S1_at | 0.24 | BQ612005 | Transporter | | | |
| 481 | Gma.11119.2.S1_s_at | 0.24 | CF920743 | Disease & Defense | | | |
| 482 | Gma.3744.1.S1_at | 0.239 | CD417438 | Unclassified - Protein With | | | |
| | | | | Unknown Function | | | |
| 483 | Gma.1137.1.S1_a_at | 0.239 | BF067710 | Metabolism | | | |
| 484 | GmaAffx.50980.1.S1_at | 0.238 | BE823095 | Signal Transduction | Protein | | |
| | | | | | Phosphatase | | |
| 485 | GmaAffx.41917.1.S1_at | 0.238 | BE658926 | Disease & Defense | | | |
| 486 | Gma.11166.1.S1_s_at | 0.237 | AW705829 | Metabolism | | | |
| 487 | GmaAffx.71308.1.S1_at | 0.236 | BF595112 | Transcription | Transcription | Heat Shock | Heat shock transcription factor |
| | | | | | Factor | | |
| 488 | Gma.398.2.S1_at | 0.236 | BU925900 | Metabolism | | | Homocysteine S-methyltransferase |
| 489 | Gma.3161.2.S1_at | 0.235 | CD400025 | Secondary Metabolism | | | Iron/ascorbate family oxidoreductases |
| 490 | GmaAffx.83818.1.S1_at | 0.235 | BU762716 | No Homology to Known Proteins | | | |
| 491 | GmaAffx.8999.2.A1_s_at | 0.234 | BI700574 | No Homology to Known Proteins | | | |
| 492 | Gma.4796.1.A1_at | 0.234 | AW102323 | Unclassified - Hypothetical Protein | | | Uncharacterized conserved protein |
| | | | | NOT Supported by cDNA | | | |
| 493 | Gma.15093.1.S1_at | 0.234 | CD400374 | Protein Destination & Storage | | | Glutathione S-transferase |
| 494 | GmaAffx.15119.1.A1_at | 0.234 | BU544528 | Metabolism | | | Possible pfkB family carbohydrate kinase |
| 495 | GmaAffx.87433.1.S1_at | 0.234 | BI701509 | Unclassified - Hypothetical Protein | | | |
| | | | | Supported by cDNA | | | |
| 496 | Gma.11166.1.S1_x_at | 0.233 | AW705829 | Metabolism | | | |
| 497 | GmaAffx.74323.1.S1_at | 0.233 | BI426587 | Unclassified - Hypothetical Protein | | | Endoplasmic reticulum protein EP58, contains |
| | | | | NOT Supported by cDNA | | | filamin rod domain and KDEL motif |
| 498 | GmaAffx.50980.1.S1_x_at | 0.232 | BE823095 | Signal Transduction | Protein | | |
| | | | | | Phosphatase | | |
| 499 | Gma.3000.1.S1_at | 0.232 | BE657172 | No Homology to Known Proteins | | | |
| 500 | Gma.4674.1.A1_at | 0.231 | AW309836 | Metabolism | | | Arylacetamide deacetylase |
| 501 | GmaAffx.47739.1.S1_at | 0.228 | BM522736 | Unclassified - Protein with | | | |
| | | | | Unknown Function | | | |
| 502 | Gma.1527.1.S1_x_at | 0.227 | BI968185 | Secondary Metabolism | | | Flavonol reductase/cinnamoyl-CoA reductase |
| 503 | Gma.18006.1.A1_at | 0.227 | AI496486 | Transcription | Transcription | Homeodomain | Transcription factor MEIS1 and related HOX |
| | | | | | Factor | | domain proteins |
| 504 | Gma.6999.1.S1_x_at | 0.225 | AF529303.1 | Disease & Defense | | | |
| 505 | GmaAffx.45003.1.S1_at | 0.224 | BI968048 | Unclassified - Hypothetical Protein | | | Uncharacterized conserved protein |
| | | | | Supported by cDNA | | | |
| 506 | GmaAffx.91338.1.S1_at | 0.222 | CF807063 | Unclassified - Hypothetical Protein | | | Predicted haloacid dehalogenase-like hydrolase |
| | | | | Supported by cDNA | | | |
| 507 | Gma.10559.1.S1_at | 0.221 | BU544359 | No Homology to Known Proteins | | | |
| 508 | Gma.5331.1.S1_at | 0.221 | BI892702 | Transcription | Transcription | NAC | |
| | | | | | Factor | Domain | |
| 509 | GmaAffx.45334.1.S1_at | 0.217 | BI969317 | Secondary Metabolism | GA | | Iron/ascorbate family oxidoreductases |
| | | | | | Biosynthesis | | |
| 510 | GmaAffx.2026.1.A1_at | 0.216 | BU550139 | Transcription | Transcription | NAC | |
| | | | | | Factor | Domain | |
| 511 | Gma.3175.1.S1_at | 0.216 | AI736154 | Unclassified - Hypothetical Protein | | | |
| | | | | NOT Supported by cDNA | | | |
| 512 | Gma.3744.2.S1_a_at | 0.215 | BI892693 | Unclassified - Protein With | | | |
| | | | | Unknown Function | | | |
| 513 | Gma.2252.1.S1_at | 0.215 | CD406598 | Unclassified - Hypothetical Protein | | | |
| | | | | Supported by cDNA | | | |
| 514 | Gma.10227.1.A1_at | 0.215 | BE020065 | Metabolism | | | Flavin-containing monooxygenase |
| 515 | GmaAffx.12933.1.S1_at | 0.211 | AW395787 | Transcription | Transcription | CCAAT | CCAAT-binding factor, subunit B (HAP2) |
| | | | | | Factor | | |
| 516 | GmaAffx.23292.1.A1_at | 0.211 | BI967795 | No Homology to Known Proteins | | | |
| 517 | Gma.7549.1.S1_at | 0.21 | BG508637 | Unclassified - Protein with | | | |
| | | | | Unknown Function | | | |
| 518 | Gma.13218.1.A1_s_at | 0.209 | CD391892 | No Homology to Known Proteins | | | |
| 519 | GmaAffx.89772.14.A1_s_at | 0.209 | CK606614 | Disease & Defense | | | |
| 520 | Gma.6921.1.S1_at | 0.208 | BE823209 | Signal Transduction | Unclassified | | Phosphatidylethanolamine binding protein |
| 521 | Gma.5611.1.S1_at | 0.208 | AW309826 | Metabolism | Cytochrome | CYP81D | Cytochrome P450 CYP2 subfamily |
| | | | | | P450 | | |
| 522 | GmaAffx.5264.1.S1_s_at | 0.207 | BM521512 | Disease & Defense | | | |
| 523 | GmaAffx.24521.1.S1_at | 0.207 | BU761767 | Unclassified - Hypothetical Protein | | | |
| | | | | NOT Supported by cDNA | | | |
| 524 | GmaAffx.10582.1.S1_at | 0.205 | BM523687 | Transcription | Transcription | CCAAT | CCAAT-binding factor, subunit A (HAP3) |
| | | | | | Factor | | |
| 525 | Gma.6033.2.S1_at | 0.204 | AI416728 | Unclassified - Hypothetical Protein | | | |
| | | | | Supported by cDNA | | | |
| 526 | GmaAffx.14030.1.S1_at | 0.204 | BE021503 | No Homology to Known Proteins | | | |
| 527 | Gma.18052.1.S1_at | 0.204 | BF595464 | Transporter | | | |
| 528 | GmaAffx.59470.1.S1_at | 0.202 | BI469872 | Secondary Metabolism | | | Phenylalanine and histidine ammonia-lyase |
| 529 | Gma.3165.1.S1_at | 0.202 | CD406755 | Transcription | Transcription | Homeodomain | Transcription factor HEX, contains HOX and |
| | | | | | Factor | | HALZ domains |
| 530 | Gma.1326.1.S1_at | 0.198 | CD397515 | Secondary Metabolism | | | |
| 531 | Gma.6192.1.S1_at | 0.197 | AW396773 | Metabolism | | | |
| 532 | GmaAffx.61593.1.S1_at | 0.197 | BU765478 | Unclassified - Hypothetical Protein | | | Sodium sulfate symporter and related arsenite |
| | | | | NOT Supported by cDNA | | | permeases |
| 533 | Gma.9638.1.A1_at | 0.195 | CA936403 | Disease & Defense | | | |
| 534 | GmaAffx.71222.1.S1_at | 0.194 | AW460050 | Metabolism | | | 5′-AMP-activated protein kinase, gamma subunit |
| 535 | GmaAffx.81618.1.S1_at | 0.194 | AW706403 | No Homology to Known Proteins | | | |
| 536 | Gma.3591.2.S1_at | 0.193 | BE473658 | Secondary Metabolism | Ethylene | | Iron/ascorbate family oxidoreductases |
| | | | | | Biosynthesis | | |
| 537 | Gma.1269.1.S1_at | 0.192 | AW309671 | Metabolism | | | Flavonol reductase/cinnamoyl-CoA reductase |
| 538 | Gma.857.1.S1_at | 0.192 | BE822264 | No Homology to Known Proteins | | | |
| 539 | Gma.6643.1.A1_at | 0.191 | BE823205 | Secondary Metabolism | | | Sulfotransferase |
| 540 | Gma.8370.1.S1_at | 0.191 | BQ454087 | Signal Transduction | Protein | | Serine/threonine protein phosphatase |
| | | | | | Phosphatase | | |
| 541 | GmaAffx.57967.1.S1_at | 0.19 | AW102170 | Protein Destination & Storage | | | |
| 542 | Gma.5469.1.S1_at | 0.19 | AW349200 | No Homology to Known Proteins | | | |
| 543 | GmaAffx.70435.2.S1_at | 0.189 | AW705853 | Transcription | Transcription | Basic Helix- | |
| | | | | | Factor | Loop-Helix | |
| | | | | | | (bHLH) | |
| 544 | Gma.6081.1.S1_at | 0.187 | BI967474 | Disease & Defense | | | Predicted chitinase |
| 545 | Gma.1394.2.51_at | 0.186 | BI470370 | Metabolism | | | |
| 546 | Gma.17877.1.S1_at | 0.185 | BQ612471 | Unclassified - Protein with | | | |
| | | | | Unknown Function | | | |
| 547 | Gma.15996.1.S1_x_at | 0.184 | BQ454193 | Unclassified - Hypothetical Protein | | | |
| | | | | Supported by cDNA | | | |
| 548 | Gma.6075.1.S1_at | 0.184 | AW317183 | Disease & Defense | | | |
| 549 | Gma.16947.1.S1_at | 0.184 | BG155375 | Unclassified - Hypothetical Protein | | | |
| | | | | Supported by cDNA | | | |
| 550 | Gma.7788.1.A1_at | 0.182 | BU549148 | Unclassified - Hypothetical Protein | | | Aspartyl protease |
| | | | | Supported by cDNA | | | |
| 551 | GmaAffx.16986.1.A1_s_at | 0.182 | AW351116 | Metabolism | | | Hydroxyindole-O-methyltransferase and related |
| | | | | | | | SAM-dependent methyltransferases |
| 552 | Gma.8489.1.S1_s_at | 0.181 | AB030493.1 | Metabolism | | | Protein involved in thiamine biosynthesis and |
| | | | | | | | DNA damage tolerance |
| 553 | Gma.1527.1.S1_a_at | 0.18 | BI968185 | Secondary Metabolism | | | Flavonol reductase/cinnamoyl-CoA reductase |
| 554 | GmaAffx.90395.1.A1_s_at | 0.177 | CF807499 | Unclassified - Protein with | | | |
| | | | | Unknown Function | | | |
| 555 | Gma.6643.2.S1_at | 0.177 | BE023121 | Secondary Metabolism | | | Sulfotransferase |
| 556 | Gma.6999.1.S1_s_at | 0.174 | AF529303.1 | Disease & Defense | | | |
| 557 | GmaAffx.5531.1.S1_at | 0.167 | BQ740711 | Cell Structure | | | |
| 558 | Gma.15534.1.S1_at | 0.162 | U50081.1 | Metabolism | | | |
| 559 | Gma.3578.1.S1_at | 0.162 | AW156522 | Disease & Defense | | | Acyl-CoA synthetase |
| 560 | Gma.6999.3.S1_s_at | 0.162 | CF921432 | Disease & Defense | | | |
| 561 | GmaAffx.59808.1.S1_at | 0.161 | BM886228 | Transporter | | | Predicted transporter (major facilitator |
| | | | | | | | superfamily) |
| 562 | GmaAffx.11897.1.S1_at | 0.16 | BE819868 | Transporter | | | Permease of the major facilitator superfamily |
| 563 | GmaAffx.295.1.A1_s_at | 0.159 | BG653267 | Protein Destination & Storage | | | |
| 564 | Gma.5309.1.S1_at | 0.158 | BM092263 | Unclassified - Hypothetical Protein | | | |
| | | | | NOT Supported by cDNA | | | |
| 565 | GmaAffx.70959.1.S1_at | 0.157 | BE611219 | Unclassified - Hypothetical Protein | | | Glucose-6-phosphate/phosphate and |
| | | | | NOT Supported by cDNA | | | phosphoenolpyruvate/phosphate antiporter |
| 566 | GmaAffx.91428.1.A1_at | 0.157 | CF807137 | Metabolism | | | |
| 567 | GmaAffx.19467.1.S1_at | 0.155 | BQ453237 | Signal Transduction | Protein | | Serine/threonine protein kinase |
| | | | | | Kinase | | |
| 568 | GmaAffx.40923.1.S1_at | 0.154 | BG653848 | Disease & Defense | | | Selenium-binding protein |
| 569 | Gma.8187.1.S1_a_at | 0.154 | BU549133 | Signal Transduction | Protein | | Serine/threonine protein phosphatase |
| | | | | | Phosphatase | | |
| 570 | Gma.9010.1.S1_at | 0.153 | CF921194 | Disease & Defense | | | |
| 571 | GmaAffx.32606.1.S1_at | 0.153 | BM270707 | Unclassified - Hypothetical Protein | | | Aquaporin (major intrinsic protein family) |
| | | | | NOT Supported by cDNA | | | |
| 572 | Gma.11731.1.A1_at | 0.15 | CD392320 | Unclassified - Protein with | | | |
| | | | | Unknown Function | | | |
| 573 | Gma.1758.1.S1_at | 0.15 | CD402046 | Unclassified - Protein with | | | |
| | | | | Unknown Function | | | |
| 574 | Gma.11028.2.S1_s_at | 0.149 | CA782711 | Metabolism | | | Glyoxylate/hydroxypyruvate reductase (D-isomer |
| | | | | | | | specific 2-hydroxy acid dehydrogenase |
| | | | | | | | superfamily) |
| 575 | Gma.4873.1.S1_at | 0.148 | BI969532 | Signal Transduction | Protein | | Serine/threonine protein kinase |
| | | | | | Kinase | | |
| 576 | Gma.15036.2.A1_at | 0.148 | CD411972 | Unclassified - Protein with | | | Predicted membrane protein |
| | | | | Unknown Function | | | |
| 577 | Gma.17662.2.S1_s_at | 0.146 | BQ630190 | Unclassified - Hypothetical Protein | | | |
| | | | | NOT Supported by cDNA | | | |
| 578 | GmaAffx.9944.1.S1_at | 0.146 | BM567882 | Cell Structure | | | |
| 579 | Gma.10216.1.S1_at | 0.145 | BI969369 | Metabolism | | | Hydroxyindole-O-methyltransferase and related |
| | | | | | | | SAM-dependent methyltransferases |
| 580 | Gma.3578.1.S1_s_at | 0.142 | AW156522 | Disease & Defense | | | Acyl-CoA synthetase |
| 581 | GmaAffx.51464.1.S1_at | 0.142 | BQ628397 | No Homology to Known Proteins | | | |
| 582 | Gma.336.1.A1_at | 0.142 | AW310815 | Metabolism | Sulfur | | |
| | | | | | Metabolism | | |
| 583 | Gma.7580.1.S1_at | 0.139 | BE057763 | Unclassified - Hypothetical Protein | | | |
| | | | | Supported by cDNA | | | |
| 584 | Gma.1445.1.S1_at | 0.138 | BQ454241 | Metabolism | | | Flavonol reductase/cinnamoyl-CoA reductase |
| 585 | Gma.12227.1.S1_at | 0.136 | BG511671 | Unclassified - Hypothetical Protein | | | |
| | | | | NOT Supported by cDNA | | | |
| 586 | GmaAffx.68507.1.S1_at | 0.136 | BI699405 | Transcription | Transcription | Heat Shock | Heat shock transcription factor |
| | | | | | Factor | | |
| 587 | GmaAffx.41230.1.A1_at | 0.135 | BI471609 | Unclassified - Hypothetical Protein | | | |
| | | | | NOT Supported by cDNA | | | |
| 588 | Gma.4260.1.S1_at | 0.134 | BM177256 | Unclassified - Hypothetical Protein | | | |
| | | | | NOT Supported by cDNA | | | |
| 589 | Gma.320.1.S1_at | 0.13 | AF019116.1 | Disease & Defense | | | |
| 590 | Gma.3810.1.S1_s_at | 0.125 | BI972063 | Disease & Defense | | | |
| 591 | Gma.6895.1.S1_a_at | 0.122 | BU545788 | Metabolism | | | Alanine-glyoxylate aminotransferase AGT2 |
| 592 | GmaAffx.12665.1.A1_at | 0.121 | BE822073 | Metabolism | | | |
| 593 | Gma.13062.1.S1_at | 0.12 | CD394315 | Metabolism | | | |
| 594 | Gma.6462.2.S1_a_at | 0.119 | BE555263 | Unclassified - Hypothetical Protein | | | |
| | | | | NOT Supported by cDNA | | | |
| 595 | GmaAffx.65393.1.S1_s_at | 0.119 | BU760770 | No Homology to Known Proteins | | | |
| 596 | Gma.3774.2.S1_at | 0.118 | CD393365 | Energy | | | Thioredoxin, nucleoredoxin and related proteins |
| 597 | Gma.2253.1.S1_at | 0.114 | AF169018.1 | Metabolism | | | Reductases with broad range of substrate |
| | | | | | | | specificities |
| 598 | GmaAffx.92479.1.S1_s_at | 0.112 | CF808204 | No Homology to Known Proteins | | | |
| 599 | Gma.17446.1.A1_at | 0.106 | AI461103 | Unclassified - Protein with | | | |
| | | | | Unknown Function | | | |
| 600 | Gma.13854.1.S1_at | 0.106 | CD408166 | Metabolism | | | |
| 601 | Gma.15.1.S1_at | 0.106 | U66316.1 | Unclassified - Protein with | | | |
| | | | | Unknown Function | | | |
| 602 | GmaAffx.87417.1.S1_at | 0.104 | BM568072 | Metabolism | | | |
| 603 | GmaAffx.52963.1.S1_at | 0.104 | AW310673 | Unclassified - Protein with | | | |
| | | | | Unknown Function | | | |
| 604 | GmaAffx.26908.1.S1_at | 0.101 | BF425834 | Secondary Metabolism | | | Beta, beta-carotene 15,15′-dioxygenase and |
| | | | | | | | related enzymes |
| 605 | Gma.17890.1.S_at | 0.101 | AW432965 | No Homology to Known Proteins | | | |
| 606 | GmaAffx.81750.1.S1_at | 0.0985 | AW306336 | No Homology to Known Proteins | | | |
| 607 | Gma.12393.3.S1_at | 0.098 | BM085488 | Secondary Metabolism | | | Beta, beta-carotene 15,15′-dioxygenase and |
| | | | | | | | related enzymes |
| 608 | GmaAffx.89407.1.A1_s_at | 0.0978 | CK606273 | Transporter | | | |
| 609 | GmaAffx.17961.1.A1_at | 0.0974 | BE657601 | Protein Destination & Storage | | | Molecular chaperones HSP70/HSC70, HSP70 |
| | | | | | | | superfamily |
| 610 | Gma.3519.1.S1_at | 0.0892 | CD403581 | Unclassified - Hypothetical Protein | | | |
| | | | | Supported by cDNA | | | |
| 611 | Gma.9740.1.S1_at | 0.0859 | BQ081058 | Unclassified - Hypothetical Protein | | | |
| | | | | Supported by cDNA | | | |
| 612 | Gma.16566.1.S1_at | 0.0856 | CD400974 | Metabolism | | | Fumarase |
| 613 | Gma.2133.1.S1_at | 0.0855 | AW310071 | Unclassified - Protein with | | | |
| | | | | Unknown Function | | | |
| 614 | Gma.1243.1.S1_at | 0.0845 | AW311027 | Unclassified - Hypothetical Protein | | | |
| | | | | Supported by cDNA | | | |
| 615 | Gma.15625.1.S1_x_at | 0.0841 | U77678.1 | Metabolism | Amino Acid | | Asparagine synthase (glutamine-hydrolyzing) |
| | | | | | Biosynthesis/ | | |
| | | | | | Metabolism | | |
| 616 | Gma.3000.2.S1_a_at | 0.0841 | BM270844 | No Homology to Known Proteins | | | |
| 617 | GmaAffx.90395.1.S1_s_at | 0.0837 | CF806120 | Unclassified - Protein with | | | |
| | | | | Unknown Function | | | |
| 618 | Gma.2986.1.S1_at | 0.0827 | BM271324 | Metabolism | | | 5′-AMP-activated protein kinase, gamma subunit |
| 619 | Gma.6192.1.S1_s_at | 0.0814 | AW396773 | Metabolism | | | |
| 620 | GmaAffx.90600.1.S1_at | 0.0806 | CF806325 | Metabolism | | | |
| 621 | Gma.15625.1.S1_a_at | 0.0771 | U77678.1 | Metabolism | Amino Acid | | Asparagine synthase (glutamine-hydrolyzing) |
| | | | | | Biosynthesis/ | | |
| | | | | | Metabolism | | |
| 622 | Gma.4687.1.S1_at | 0.0751 | AW309716 | Secondary Metabolism | | | |
| 623 | Gma.11211.1.S1_s_at | 0.074 | AI442578 | No Homology to Known Proteins | | | |
| 624 | GmaAffx.24413.1.A1_at | 0.0704 | BU548368 | Metabolism | | | 5′-AMP-activated protein kinase, gamma subunit |
| 625 | Gma.8365.1.S1_at | 0.07 | CD396813 | Unclassified - Hypothetical Protein | | | |
| | | | | NOT Supported by cDNA | | | |
| 626 | Gma.2912.1.S1_s_at | 0.0691 | AF117884.1 | Protein Destination & Storage | Storage | | Uncharacterized conserved protein |
| | | | | | Protein | | |
| 627 | Gma.17985.1.S1_at | 0.0678 | CD416450 | Metabolism | | | |
| 628 | GmaAffx.73698.1.S1_at | 0.0673 | BU964780 | Metabolism | | | 5′-AMP-activated protein kinase, gamma subunit |
| 629 | GmaAffx.90600.1.S1_s_at | 0.065 | CF806325 | Metabolism | | | |
| 630 | GmaAffx.6635.1.S1_at | 0.0639 | CD412718 | Disease & Defense | | | |
| 631 | Gma.17516.1.S1_at | 0.0631 | CD396417 | Transporter | | | Ca2+/H+ antiporter VCX1 and related proteins |
| 632 | Gma.1527.2.S1_at | 0.0624 | BE658017 | Secondary Metabolism | | | Flavonol reductase/cinnamoyl-CoA reductase |
| 633 | Gma.176.1.S1_s_at | 0.0618 | AF004810.1 | Protein Destination & Storage | Storage | | |
| | | | | | Protein | | |
| 634 | Gma.1332.1.S1_x_at | 0.0609 | BI970666 | Secondary Metabolism | Ethylene | | 1-aminocyclopropane-1-carboxylate synthase, |
| | | | | | Biosynthesis | | and related proteins |
| 635 | Gma.9947.1.S1_at | 0.0589 | BF324269 | Unclassified - Hypothetical Protein | | | |
| | | | | Supported by cDNA | | | |
| 636 | Gma.8425.1.S1_s_at | 0.0562 | D83968.1 | Metabolism | Cytochrome | CYP93D | Cytochrome P450 CYP2 subfamily |
| | | | | | P450 | | |
| 637 | Gma.8318.1.A1_at | 0.0544 | BU550469 | No Homology to Known Proteins | | | |
| 638 | Gma.2912.1.S1_at | 0.0517 | AF117884.1 | Protein Destination & Storage | Storage | | Uncharacterized conserved protein |
| | | | | | Protein | | |
| 639 | Gma.79.1.S1_x_at | 0.0493 | S44202.1 | Unclassified - Protein with | | | |
| | | | | Unknown Function | | | |
| 640 | Gma.14471.1.S1_s_at | 0.0479 | CF921241 | No Homology to Known Proteins | | | |
| 641 | Gma.1314.1.S1_at | 0.0462 | U63726.1 | Metabolism | | | Gamma-glutamyl hydrolase |
| 642 | GmaAffx.19541.1.S1_s_at | 0.044 | BQ612990 | No Homology to Known Proteins | | | |
| 643 | GmaAffx.10204.1.S1_at | 0.0421 | BQ298213 | Unclassified - Hypothetical Protein | | | |
| | | | | Supported by cDNA | | | |
| 644 | Gma.5947.1.S1_s_at | 0.0413 | BQ610509 | Disease & Defense | | | |
| 645 | Gma.3774.1.S1_at | 0.0383 | BU550698 | Energy | | | Thioredoxin, nucleoredoxin and related proteins |
| 646 | Gma.79.1.S1_s_at | 0.0381 | S44202.1 | Unclassified - Protein with | | | |
| | | | | Unknown Function | | | |
| 647 | GmaAffx.68621.1.A1_at | 0.0301 | BI893889 | Unclassified - Protein with | Stress | | |
| | | | | Unknown Function | Induced | | |
| 648 | GmaAffx.85382.1.S1_s_at | 0.0234 | BI945154 | No Homology to Known Proteins | | | |
| 649 | Gma.3774.2.S1_a_at | 0.0216 | CD393365 | Energy | | | Thioredoxin, nucleoredoxin and related proteins |
| 650 | Gma.15307.1.S1_at | 0.0127 | U64866.1 | Disease & Defense | | | |

TABLE 10

| ID | Gene Name | Fold Change | Soybean_GenBank_Acc | Functional Category | Sub-Family | Gene Family | Additional Description |
|---|---|---|---|---|---|---|---|
| 1 | Gma.10892.1.S1_a_at | 14.15 | BG839570 | Metabolism | | | Predicted carbonic anhydrase involved in |
| | | | | | | | protection against oxidative damage |
| 2 | GmaAffx.42583.1.A1_at | 8.466 | BG154898 | Transporter | lnorganic | Fe(II) | Fe2+/Zn2+ regulated transporter |
| | | | | | Solute | transporter | |
| | | | | | Cotransporters | isolog family | |
| 3 | GmaAffx.14016.1.S1_at | 8.082 | BE824450 | No Homology to Known Proteins | | | |
| 4 | Gma.964.1.A1_at | 5.456 | BE824146 | Metabolism | | | Tyrosine aminotransferase |
| 5 | Gma.5139.1.S1_at | 4.589 | AF243376.1 | Secondary Metabolism | | | Glutathione S-transferase |
| 6 | GmaAffx.3369.1.S1_at | 2.835 | BU548983 | Cell Structure | | | |
| 7 | Gma.3581.2.S1_a_at | 2.671 | CD398783 | Unclassified - Hypothetical | | | Ca2+-dependent lipid-binding protein |
| | | | | Protein NOT Supported by | | | CLB1/vesicle protein vp115/Granuphilin A, |
| | | | | cDNA | | | contains C2 domain |
| 8 | Gma.17321.1.S1_at | 2.572 | AW508844 | Metabolism | | | Tyrosine aminotransferase |
| 9 | Gma.7301.1.S1_at | 2.472 | BM528250 | Disease & Defense | | | Gamma-glutamyltransferase |
| 10 | Gma.7608.1.S1_at | 2.247 | BU549027 | Unclassified - Hypothetical | | | RNA-binding protein Sam68 and related KH |
| | | | | Protein NOT Supported by cDNA | | | domain proteins |
| 11 | GmaAffx.6571.1.S1_at | 2.238 | CD403804 | Metabolism | | | UDP-glucuronosyl and UDP-glucosyl |
| | | | | | | | transferase |
| 12 | GmaAffx.40114.1.A1_at | 2.199 | BE661587 | Unclassified - Hypothetical | | | |
| | | | | Protein Supported by cDNA | | | |
| 13 | Gma.16521.1.S1_at | 2.185 | BE609300 | Unclassified - Hypothetical | | | Uncharacterized conserved protein |
| | | | | Protein Supported by cDNA | | | |
| 14 | GmaAffx.934.1.S1_at | 2.132 | BG046802 | No Homology to Known | | | |
| | | | | Proteins | | | |
| 15 | Gma.5124.1.S1_at | 2.128 | BI971079 | Protein Destination & Storage | | | |
| 16 | Gma.4374.1.S1_at | 2.08 | BM178538 | Metabolism | Cytochrome | CYP76C | Cytochrome P450 CYP2 subfamily |
| | | | | | P450 | | |
| 17 | GmaAffx.6.1.S1_at | 2.07 | AW348562 | No Homology to Known | | | |
| | | | | Proteins | | | |
| 18 | Gma.12212.1.A1_s_at | 2.053 | CD396776 | No Homology to Known | | | |
| | | | | Proteins | | | |
| 19 | Gma.3136.1.S1_at | 2.04 | BG839241 | Metabolism | Cytochrome | CYP82C | Cytochrome P450 CYP2 subfamily |
| | | | | | P450 | | |
| 20 | Gma.2194.1.S1_at | 2.038 | BE657352 | Unclassified - Hypothetical | | | |
| | | | | Protein Supported by cDNA | | | |
| 21 | Gma.12641.1.A1_at | 0.5 | CD393495 | No Homology to Known | | | |
| | | | | Proteins | | | |
| 22 | Gma.11665.2.A1_at | 0.498 | BU545959 | No Homology to Known | | | |
| | | | | Proteins | | | |
| 23 | GmaAffx.9400.2.S1_at | 0.497 | BI945484 | Signal Transduction | Protein Kinase | | Glycerophosphoryl diester phosphodiesterase |
| 23 | GmaAffx.9400.2.S1_at | 0.497 | BI945484 | Signal Transduction | Protein Kinase | | Serine/threonine protein kinase |
| 24 | GmaAffx.60958.1.S1_at | 0.494 | BG551038 | Transcription | Transcription | Basic Helix- | |
| | | | | | Factor | Loop-Helix | |
| | | | | | | (bHLH) | |
| 25 | GmaAffx.37484.1.S1_at | 0.494 | BG651852 | Unclassified - Protein with | | | Predicted membrane protein, contains DoH |
| | | | | Unknown Function | | | and Cytochrome b-561/ferric reductase |
| | | | | | | | transmembrane domains |
| 26 | GmaAffx.81585.1.S1_at | 0.493 | BM187887 | Unclassified - Hypothetical | | | Copper chaperone |
| | | | | Protein NOT Supported by | | | |
| | | | | cDNA | | | |
| 27 | GmaAffx.43774.1.A1_at | 0.492 | AW349173 | Cell Structure | | | |
| 28 | Gma.1704.1.S1_at | 0.492 | AW349971 | Metabolism | | | Glutaredoxin and related proteins |
| 29 | GmaAffx.3697.1.S1_at | 0.492 | BE820647 | Disease & Defense | | | |
| 30 | GmaAffx.72479.1.S1_at | 0.491 | BG043825 | Transcription | Transcription | AP2/EREBP | Uncharacterized conserved protein |
| | | | | | Factor | | |
| 31 | GmaAffx.47228.1.S1_at | 0.491 | BU761053 | Transporter | | | |
| 32 | Gma.15907.2.S1_at | 0.491 | BE329550 | Signal Transduction | Unclassified | | FOG: Leucine rich repeat |
| 33 | PsAffx.psHB026xH01f_s_at | 0.49 | | | | | |
| 34 | Gma.11195.1.S1_at | 0.49 | CD417406 | Transporter | | | Apolipoprotein D/Lipocalin |
| 35 | Gma.4680.1.A1_s_at | 0.489 | AW309707 | Unclassified - Protein with | | | |
| | | | | Unknown Function | | | |
| 36 | GmaAffx.84795.1.S1_at | 0.489 | BG046191 | Unclassified - Hypothetical | | | |
| | | | | Protein NOT Supported by | | | |
| | | | | cDNA | | | |
| 37 | Gma.9013.1.S1_at | 0.488 | AF243368.1 | Disease & Defense | | | Glutathione S-transferase |
| 38 | GmaAffx.71589.1.S1_at | 0.487 | BE020161 | Cell Growth & Division | | | |
| 39 | GmaAffx.93189.1.S1_at | 0.487 | CF808914 | Metabolism | | | Carbon-nitrogen hydrolase |
| 40 | GmaAffx.75219.1.S1_at | 0.486 | BE020773 | Unclassified - Hypothetical | | | Fasciclin and related adhesion glycoproteins |
| | | | | Protein NOT Supported by | | | |
| | | | | cDNA | | | |
| 41 | GmaAffx.83564.1.S1_at | 0.485 | BU578309 | No Homology to Known | | | |
| | | | | Proteins | | | |
| 42 | GmaAffx.40086.1.S1_at | 0.485 | BE6598I5 | Unclassified - Hypothetical | | | |
| | | | | Protein Supported by cDNA | | | |
| 43 | Gma.6129.2.S1_s_at | 0.484 | AW186006 | Protein Synthesis | | | Translation initiation factor 5A (eIF-5A) |
| 44 | Gma.1715.1.S1_at | 0.483 | BE191618 | Metabolism | | | |
| 45 | GmaAffx.72986.1.S1_at | 0.483 | BI317536 | Metabolism | | | Inositol polyphosphate 5-phosphatase and |
| | | | | | | | related proteins |
| 46 | GmaAffx.89049.1.S1_at | 0.483 | CK606168 | Intracellular Traffic | | | Annexin |
| 47 | GmaAffx.26967.1.S1_at | 0.482 | BE022958 | Unclassified - Hypothetical | | | |
| | | | | Protein Supported by cDNA | | | |
| 48 | GmaAffx.28578.1.S1_at | 0.482 | BQ094224 | Disease & Defense | | | |
| 49 | GmaAffx.90331.1.S1_s_at | 0.482 | CF806056 | Disease & Defense | | | |
| 50 | Gma.5889.1.S1_at | 0.481 | BQ610202 | Unclassified - Hypothetical | | | Endoplasmic reticulum protein EP58, |
| | | | | Protein NOT Supported by | | | contains filamin rod domain and KDEL motif |
| | | | | cDNA | | | |
| 51 | Gma.1137.2.S1_x_at | 0.481 | CA852403 | Metabolism | | | |
| 52 | Gma.3591.1.S1_x_at | 0.481 | BE440266 | Secondary Metabolism | Ethylene | | Iron/ascorbate family oxidoreductases |
| | | | | | Biosynthesis | | |
| 53 | Gma.7970.1.S1_at | 0.481 | BI699189 | No Homology to Known | | | |
| | | | | Proteins | | | |
| 54 | GmaAffx.28539.1.S1_at | 0.481 | BQ080184 | Disease & Defense | | | Ca2+-independent phospholipase A2 |
| 55 | GmaAffx.30428.1.S1_at | 0.48 | BE824410 | Protein Destination & Storage | | | Molecular chaperones HSP70/HSC70, |
| | | | | | | | HSP70 superfamily |
| 56 | Gma.16585.1.S1_x_at | 0.48 | CA850597 | Metabolism | | | Predicted carbonic anhydrase involved in |
| | | | | | | | protection against oxidative damage |
| 57 | Gma.8160.1.S1_at | 0.48 | BI787214 | Unclassified - Hypothetical | | | |
| | | | | Protein NOT Supported by | | | |
| | | | | cDNA | | | |
| 58 | GmaAffx.87670.1.S1_at | 0.48 | BQ452640 | Unclassified - Hypothetical | | | |
| | | | | Protein NOT Supported by | | | |
| | | | | cDNA | | | |
| 59 | Gma.4120.1.A1_at | 0.479 | AW310714 | Unclassified - Protein with | | | |
| | | | | Unknown Function | | | |
| 60 | Gma.6630.1.S1_s_at | 0.478 | BG652851 | Unclassified - Hypothetical | | | |
| | | | | Protein Supported by cDNA | | | |
| 61 | GmaAffx.81584.1.S1_at | 0.478 | BI426969 | Disease & Defense | | | |
| 62 | GmaAffx.69076.1.S1_at | 0.477 | BM525610 | Unclassified - Hypothetical | | | |
| | | | | Protein NOT Supported by | | | |
| | | | | cDNA | | | |
| 63 | Gma.8240.1.S1_at | 0.476 | BM092761 | Secondary Metabolism | | | lron/ascorbate family oxidoreductases |
| 64 | GmaAffx.30665.1.A1_at | 0.476 | BQ453743 | Signal Transduction | Protein Kinase | | Serine/threonine protein kinase |
| 65 | Gma.16526.1.S1_at | 0.475 | CD407998 | Protein Destination & Storage | | | Serine carboxypeptidases (lysosomal |
| | | | | | | | cathepsin A) |
| 66 | GmaAffx.16207.1.S1_at | 0.475 | BE821188 | Cell Structure | | | Beta-glucosidase, lactase phlorizinhydrolase, |
| | | | | | | | and related proteins |
| 67 | GmaAffx.8712.1.S1_s_at | 0.474 | AW349107 | Disease & Defense | | | |
| 68 | GmaAffx.46684.1.S1_at | 0.474 | BQ081034 | Signal Transduction | Protein Kinase | | |
| 69 | Gma.13000.1.S1_at | 0.474 | CD415111 | Unclassified - Hypothetical | | | |
| | | | | Protein Supported by cDNA | | | |
| 70 | Gma.285.1.S1_at | 0.472 | BG726992 | Cell Growth & Division | | | |
| 71 | Gma.10824.1.S1_at | 0.472 | BE824257 | Unclassified - Hypothetical | | | Magnesium transporters: CorA family |
| | | | | Protein Supported by cDNA | | | |
| 72 | GmaAffx.91641.1.S1_s_at | 0.472 | CF807366 | Disease & Defense | | | Acyl-CoA synthetase |
| 73 | Gma.4610.1.S1_s_at | 0.472 | BM522856 | No Homology to Known | | | |
| | | | | Proteins | | | |
| 74 | GmaAffx.86136.1.S1_at | 0.471 | AI440673 | Unclassified - Protein with | | | |
| | | | | Unknown Function | | | |
| 75 | Gma.6498.1.A1_at | 0.471 | BE824396 | Unclassified - Hypothetical | | | |
| | | | | Protein Supported by cDNA | | | |
| 76 | GmaAffx.92136.1.S1_s_at | 0.471 | CF807451 | Signal Transduction | Protein Kinase | | Mannosyltransferase |
| 77 | Gma.1332.1.S1_s_at | 0.468 | BI970666 | Secondary Metabolism | Ethylene | | 1-aminocyclopropane-1-carboxylate |
| | | | | | Biosynthesis | | synthase, and related proteins |
| 78 | GmaAffx.4936.1.S1_at | 0.467 | AW666270 | Transporter | | | Multidrug/pheromone exporter. ABC |
| | | | | | | | superfamily |
| 79 | Gma.3283.1.S1_at | 0.465 | BQ298733 | No Homology to Known | | | |
| | | | | Proteins | | | |
| 80 | GmaAffx.89049.1.S1_s_at | 0.464 | CK606168 | Intracellular Traffic | | | Annexin |
| 81 | Gma.10026.1.S1_at | 0.464 | BU546301 | Unclassified - Protein with | | | |
| | | | | Unknown Function | | | |
| 82 | Gma.16896.1.A1_at | 0.462 | CD416188 | No Homology to Known | | | |
| | | | | Proteins | | | |
| 83 | Gma.1980.1.A1_at | 0.462 | BM520466 | Signal Transduction | Protein Kinase | AtC1PKs | Serine/threonine protein kinase |
| 84 | GmaAffx.49130.1.S1_at | 0.462 | BE555035 | No Homology to Known | | | |
| | | | | Proteins | | | |
| 85 | Gma.1528.1.S1_at | 0.461 | CD403623 | Transcription | Transcription | Basic Helix- | |
| | | | | | Factor | Loop-Helix | |
| | | | | | | (bHLH) | |
| 86 | GmaAffx.86392.1.S1_at | 0.46 | AI736754 | Signal Transduction | Calcium- | | |
| | | | | | Binding | | |
| 87 | Gma.10581.1.S1_at | 0.46 | AW396439 | Unclassified - Hypothetical | | | Predicted haloacid dehalogenase-like |
| | | | | Protein Supported by cDNA | | | hydrolase |
| 88 | GmaAffx.65419.1.S1_at | 0.459 | CD405955 | Signal Transduction | Protein Kinase | | |
| 89 | Gma.412.1.S1_at | 0.459 | BE607835 | Protein Destination & Storage | | | Ubiquitin-protein ligase |
| 90 | Gma.1839.2.S1_a_at | 0.459 | AW507949 | Metabolism | | | |
| 91 | Gma.17288.1.A1_at | 0.458 | AW310692 | No Homology to Known | | | |
| | | | | Proteins | | | |
| 92 | Gma.850.1.S1_at | 0.458 | BE822050 | Unclassified - Hypothetical | | | |
| | | | | Protein NOT Supported by | | | |
| | | | | cDNA | | | |
| 93 | GmaAffx.74868.1.S1_at | 0.457 | CA820573 | No Homology to Known | | | |
| | | | | Proteins | | | |
| 94 | GmaAffx.29254.1.S1_at | 0.456 | AW760361 | Transcription | Transcription | Basic Helix- | |
| | | | | | Factor | Loop-Helix | |
| | | | | | | (bHLH) | |
| 95 | Gma.4394.1.S1_a_at | 0.456 | CD409273 | Transcription | Transcription | Homeodomain | Transcription factor HEX, contains HOX and |
| | | | | | Factor | | HALZ domains |
| 96 | Gma.17759.1.S1_at | 0.456 | BU927041 | Transcription | Transcription | AUX/1AA | |
| | | | | | Factor | | |
| 97 | Gma.10393.1.S1_at | 0.455 | BE804608 | Unclassified - Hypothetical | | | Endoplasmic reticulum protein EP58, |
| | | | | Protein NOT Supported by | | | contains filamin rod domain and KDEL motif |
| | | | | cDNA | | | |
| 98 | GmaAffx.90789.1.S1_s_at | 0.454 | CF806514 | Unclassified - Protein with | | | |
| | | | | Unknown Function | | | |
| 99 | GmaAffx.67113.1.S1_at | 0.454 | BE329541 | No Homology to Known | | | |
| | | | | Proteins | | | |
| 100 | GmaAffx.51444.1.S1_at | 0.453 | BM178639 | Secondary Metabolism | | | Multicopper oxidases |
| 101 | Gma.1068.1.S1_at | 0.453 | L19360.1 | Signal Transduction | Protein Kinase | | Serine/threonine protein kinase |
| 102 | GmaAffx.44471.1.A1_at | 0.452 | CA851833 | Unclassified - Hypothetical | | | |
| | | | | Protein NOT Supported by | | | |
| | | | | cDNA | | | |
| 103 | GmaAffx.9145.1.S1_s_at | 0.452 | BU763814 | Disease & Defense | | | |
| 104 | Gma.3531.1.S1_s_at | 0.452 | BI498803 | Unclassified - Hypothetical | | | FOG: PPR repeat |
| | | | | Protein Supported by cDNA | | | |
| 105 | GmaAffx.84209.1.S1_at | 0.452 | BQ610588 | Signal Transduction | Histidine | | Sensory transduction histidine kinase |
| | | | | | Kinase | | |
| 106 | Gma.16940.1.S1_at | 0.451 | AW310039 | Transcription | Transcription | Homeodomain | Transcription factor MEIS1 and related HOX |
| | | | | | Factor | | domain proteins |
| 107 | Gma.1263.1.S1_at | 0.449 | AY144180.1 | Metabolism | | | Ca2+/calmodulin-dependent protein kinase, |
| | | | | | | | EF-Hand protein superfamily |
| 108 | GmaAffx.72346.1.S1_at | 0.448 | BF424359 | Unclassified - Hypothetical | | | Speckle-type POZ protein SPOP and related |
| | | | | Protein NOT Supported by | | | proteins with TRAF, MATH and BTB/POZ |
| | | | | cDNA | | | domains |
| 109 | Gma.10144.2.S1_at | 0.447 | BU080819 | Metabolism | | | |
| 110 | GmaAffx.9040.1.S1_at | 0.446 | AW101738 | Unclassified - Hypothetical | | | |
| | | | | Protein Supported by cDNA | | | |
| 111 | Gma.3893.3.S1_at | 0.446 | BG651854 | Unclassified - Hypothetical | | | |
| | | | | Protein Supported by cDNA | | | |
| 112 | GmaAffx.92184.1.S1_s_at | 0.445 | CF807909 | Unclassified - Protein with | | | |
| | | | | Unknown Function | | | |
| 113 | Gma.6264.1.S1_at | 0.444 | BQ612558 | Unclassified - Hypothetical | | | |
| | | | | Protein Supported by cDNA | | | |
| 114 | GmaAffx.86514.1.S1_at | 0.444 | BG790017 | Transcription | Transcription | ARF | |
| | | | | | Factor | | |
| 115 | GmaAffx.93182.1.S1_s_at | 0.443 | CF808907 | Secondary Metabolism | Ethylene | | Iron/ascorbate family oxidoreductases |
| | | | | | Biosynthesis | | |
| 116 | GmaAffx.58073.1.S1_at | 0.443 | CA852105 | Unclassified - Hypothetical | | | |
| | | | | Protein Supported by cDNA | | | |
| 117 | Gma.11028.1.S1_s_at | 0.443 | BE823084 | Metabolism | | | Glyoxylate/hydroxypyruvate reductase (D- |
| | | | | | | | isomer-specific 2-hydroxy acid |
| | | | | | | | dehydrogenase superfamily) |
| 118 | Gma.6550.2.S1_s_at | 0.442 | BM086044 | Metabolism | | | Hydroxyindole-O-methyltransferase and |
| | | | | | | | related SAM-dependent methyltransferases |
| 119 | GmaAffx.91266.1.S1_s_at | 0.442 | CF806991 | Unclassified - Hypothetical | | | |
| | | | | Protein Supported by cDNA | | | |
| 120 | GmaAffx.18880.1.S1_at | 0.442 | BM094235 | Signal Transduction | Protein Kinase | | Serine/threonine protein kinase |
| 121 | Gma.3579.1.S1_at | 0.442 | AW349799 | Unclassified - Hypothetical | | | Kelch repeat-containing proteins |
| | | | | Protein Supported by cDNA | | | |
| 122 | Gma.14471.4.S1_x_at | 0.441 | BQ081409 | Transporter | | | Aquaporin (major intrinsic protein family) |
| 123 | GmaAffx.65140.1.S1_at | 0.441 | BI700031 | No Homology to Known | | | |
| | | | | Proteins | | | |
| 124 | Gma.1895.1.A1_at | 0.44 | AW309584 | Protein Destination & Storage | | | ER lumen protein retaining receptor |
| 125 | Gma.16528.1.S1_at | 0.44 | CD398706 | Unclassified - Hypothetical | | | |
| | | | | Protein Supported by cDNA | | | |
| 126 | Gma.10950.1.S1_at | 0.44 | BI699798 | Unclassified - Hypothetical | | | |
| | | | | Protein Supported by cDNA | | | |
| 127 | Gma.15996.1.S1_at | 0.439 | BQ454193 | Unclassified - Hypothetical | | | |
| | | | | Protein Supported by cDNA | | | |
| 128 | GmaAffx.55953.1.S1_at | 0.439 | BQ629142 | Unclassified - Hypothetical | | | FOG: Predicted E3 ubiquitin ligase |
| | | | | Protein NOT Supported by | | | |
| | | | | cDNA | | | |
| 129 | GmaAffx.93553.1.S1_s_at | 0.439 | CF809278 | Metabolism | | | |
| 130 | GmaAffx.74194.1.S1_at | 0.438 | BI968058 | Metabolism | | | 17 beta-hydroxysteroid dehydrogenase type |
| | | | | | | | 3, HSD17B3 |
| 131 | GmaAffx.87793.2.S1_at | 0.437 | BQ740671 | Signal Transduction | Protein Kinase | | Glycogen synthase kinase-3 |
| 132 | GmaAffx.42856.1.S1_at | 0.435 | BM567981 | Transcription | Transcription | Basic Helix- | |
| | | | | | Factor | Loop-Helix | |
| | | | | | | (bHLH) | |
| 133 | GmaAffx.1301.78.S1_s_at | 0.435 | AW394497 | No Homology to Known | | | |
| | | | | Proteins | | | |
| 134 | Gma.11334.1.S1_a_at | 0.433 | BI967327 | Cell Structure | | | O-methyltransferase |
| 135 | Gma.5559.1.S1_s_at | 0.433 | BM121524 | Unclassified - Hypothetical | | | |
| | | | | Protein Supported by cDNA | | | |
| 136 | GmaAffx.92230.1.A1_s_at | 0.432 | CF807955 | Disease & Defense | | | |
| 137 | Gma.398.1.A1_at | 0.43 | AW350679 | Metabolism | | | Homocysteine S-methyltransferase |
| 138 | Gma.4665.1.A1_at | 0.43 | AW309815 | No Homology to Known | | | |
| | | | | Proteins | | | |
| 139 | Gma.10969.1.S1_a_at | 0.426 | U36191.1 | Metabolism | | | |
| 140 | Gma.2080.2.S1_a_at | 0.424 | BM520916 | Secondary Metabolism | | | |
| 141 | Gma.8514.1.S1_at | 0.422 | CA851666 | Protein Destination & Storage | | | Glutathione S-transferase |
| 142 | GmaAffx.91328.1.S1_at | 0.421 | CF807053 | Unclassified - Hypothetical | | | Leucine rich repeat proteins, some proteins |
| | | | | Protein Supported by cDNA | | | contain F-box |
| 143 | Gma.6278.1.S1_at | 0.419 | BQ612647 | Unclassified - Hypothetical | | | |
| | | | | Protein Supported by cDNA | | | |
| 144 | GmaAffx.487.1.S1_at | 0.419 | BG511495 | Transporter | | | Predicted transporter (major facilitator |
| | | | | | | | superfamily) |
| 145 | GmaAffx.84206.1.S1_at | 0.418 | BU760571 | Transcription | Transcription | NAC Domain | |
| | | | | | Factor | | |
| 146 | GmaAffx.47230.2.S1_at | 0.417 | BG652486 | Transporter | | | Amino acid transporters |
| 147 | Gma.11277.2.S1_at | 0.416 | BI701238 | Unclassified - Hypothetical | | | |
| | | | | Protein NOT Supported by | | | |
| | | | | cDNA | | | |
| 148 | Gma.5400.1.S1_at | 0.416 | BM093775 | No Homology to Known | | | |
| | | | | Proteins | | | |
| 149 | GmaAffx.33121.1.S1_at | 0.416 | BQ611246 | Unclassified - Hypothetical | | | |
| | | | | Protein Supported by cDNA | | | |
| 150 | Gma.6550.2.S1_x_at | 0.413 | BM086044 | Metabolism | | | Hydroxyindole-O-methyltransferase and |
| | | | | | | | related SAM-dependent methyltransferases |
| 151 | GmaAffx.39703.1.S1_at | 0.413 | BE023656 | Protein Destination & Storage | | | Serine carboxypeptidases (lysosomal |
| | | | | | | | cathepsin A) |
| 152 | GmaAffx.15222.1.S1_at | 0.413 | BU550700 | Unclassified - Hypothetical | | | |
| | | | | Protein NOT Supported by | | | |
| | | | | cDNA | | | |
| 153 | GmaAffx.20146.1.S1_at | 0.413 | BE824274 | Unclassified - Protein with | | | Ca2+-binding protein (centrin/caltractin), EF- |
| | | | | Unknown Function | | | Hand superfamily protein |
| 154 | GmaAffx.71016.1.S1_at | 0.411 | AW234455 | Unclassified - Hypothetical | | | Arylacetamide deacetylase |
| | | | | Protein Supported by cDNA | | | |
| 155 | GmaAffx.91141.1.S1_at | 0.411 | CF806866 | Disease & Defense | | | |
| 156 | Gma.16196.1.S1_at | 0.41 | AW350888 | Transcription | Transcription | Unclassified | |
| | | | | | Factor | | |
| 157 | GmaAffx.23289.1.S1_at | 0.409 | CD392087 | No Homology to Known | | | |
| | | | | Proteins | | | |
| 158 | GmaAffx.93592.1.S1_x_at | 0.409 | CF809317 | Unclassified - Hypothetical | | | |
| | | | | Protein Supported by cDNA | | | |
| 159 | GmaAffx.92774.1.S1_at | 0.408 | CF808124 | Cell Structure | | | |
| 160 | GmaAffx.478.2.S1_at | 0.406 | BU763963 | Transcription | Transcription | Homeodomain | Transcription factor MEIS1 and related HOX |
| | | | | | Factor | | domain proteins |
| 161 | GmaAffx.24368.1.A1_at | 0.405 | BU545331 | No Homology to Known | | | |
| | | | | Proteins | | | |
| 162 | GmaAffx.93373.1.S1_s_at | 0.403 | CF809098 | Unclassified - Hypothetical | | | |
| | | | | Protein Supported by cDNA | | | |
| 163 | GmaAffx.64037.1.S1_at | 0.403 | BI321162 | Metabolism | Cytochrome | CYP82F | Cytochrome P450 CYP2 subfamily |
| | | | | | P450 | | |
| 164 | Gma.6709.1.S1_at | 0.402 | BE823291 | Signal Transduction | Protein Kinase | AtCIPKs | Serine/threonine protein kinase |
| 165 | Gma.17015.1.S1_at | 0.402 | AW733363 | No Homology to Known | | | |
| | | | | Proteins | | | |
| 166 | GmaAffx.88675.1.A1_at | 0.4 | CD417704 | Unclassified - Hypothetical | | | |
| | | | | Protein NOT Supported by | | | |
| | | | | cDNA | | | |
| 167 | Gma.5294.1.S1_s_at | 0.398 | BE658252 | Unclassified - Hypothetical | | | |
| | | | | Protein Supported by cDNA | | | |
| 168 | Gma.3776.1.S1_at | 0.398 | AW780736 | Metabolism | | | Glutaredoxin and related proteins |
| 169 | GmaAffx.44396.1.S1_at | 0.398 | BE330360 | No Homology to Known | | | |
| | | | | Proteins | | | |
| 170 | GmaAffx.80393.1.A1_at | 0.397 | BE658680 | No Homology to Known | | | |
| | | | | Proteins | | | |
| 171 | Gma.6075.1.S1_a_at | 0.396 | AW317183 | Disease & Defense | | | |
| 172 | GmaAffx.62364.1.S1_at | 0.395 | AW733764 | Protein Destination & Storage | | | Dipeptidyl aminopeptidase |
| 173 | GmaAffx.80900.1.S1_at | 0.393 | BE347494 | Metabolism | | | Iron/ascorbate family oxidoreductases |
| 174 | GmaAffx.79390.3.S1_at | 0.393 | BU082743 | Transcription | Transcription | Basic Leucine | FOG: Armadillo/beta-catenin-like repeats |
| | | | | | Factor | Zipper (bZIP) | |
| 175 | GmaAffx.4888.1.A1_at | 0.392 | BI967635 | No Homology to Known | | | |
| | | | | Proteins | | | |
| 176 | Gma.6727.1.S1_at | 0.39 | Y10493.1 | Metabolism | Cytochrome | CYP71B | Cytochrome P450 CYP2 subfamily |
| | | | | | P450 | | |
| 177 | GmaAffx.79390.1.S1_at | 0.39 | BU550458 | Transcription | Transcription | Basic Leucine | FOG: Armadillo/beta-catenin-like repeats |
| | | | | | Factor | Zipper (bZIP) | |
| 178 | GmaAffx.47590.1.S1_at | 0.39 | CA853861 | Metabolism | | | UDP-glucuronosyl and UDP-glucosyl |
| | | | | | | | transferase |
| 179 | GmaAffx.99.4.S1_at | 0.39 | BQ742171 | Unclassified - Protein with | | | Microtubule-binding protein (translationally |
| | | | | Unknown Function | | | controlled tumor protein) |
| 180 | Gma.4822.1.S1_at | 0.388 | BI969249 | Disease & Defense | | | |
| 181 | GmaAffx.63822.1.S1_at | 0.388 | BM520838 | Disease & Defense | | | Purple (tartrate-resistant) acid phosphatase |
| 182 | Gma.10827.2.S1_s_at | 0.386 | BQ612452 | Protein Destination & Storage | Storage Protein | | |
| 183 | GmaAffx.82000.1.S1_at | 0.385 | BI094895 | Unclassified - Hypothetical | | | |
| | | | | Protein Supported by cDNA | | | |
| 184 | GmaAffx.24451.1.A1_at | 0.384 | BU550405 | Metabolism | Cytochrome | CYP90C | Cytochrome P450 CYP4/CYP19/CYP26 |
| | | | | | P450 | | subfamilies |
| 185 | GmaAffx.85657.1.S1_s_at | 0.384 | AW185911 | Unclassified - Protein with | | | FOG: Kelch repeat |
| | | | | Unknown Function | | | |
| 186 | Gma.13736.1.A1_at | 0.384 | CD404408 | Secondary Metabolism | | | Multicopper oxidases |
| 187 | GmaAffx.53108.1.S1_at | 0.384 | BM093748 | Signal Transduction | Protein Kinase | | Serine/threonine protein kinase |
| 188 | Gma.16875.1.A1_at | 0.383 | CD406723 | Metabolism | | | Aldo/keto reductase family proteins |
| 189 | GmaAffx.44484.1.S1_at | 0.383 | BE475213 | Unclassified - Hypothetical | | | |
| | | | | Protein NOT Supported by | | | |
| | | | | cDNA | | | |
| 190 | Gma.2376.1.S1_at | 0.382 | CA852095 | Transcription | Transcription | Homeodomain | Transcription factor MEIS1 and related HOX |
| | | | | | Factor | | domain proteins |
| 191 | Gma.1642.3.S1_s_at | 0.382 | BM891895 | Unclassified - Hypothetical | | | |
| | | | | Protein Supported by cDNA | | | |
| 192 | Gma.16842.1.A1_at | 0.382 | CD392652 | No Homology to Known | | | |
| | | | | Proteins | | | |
| 193 | Gma.2686.1.S1_s_at | 0.381 | BI970455 | Secondary Metabolism | | | |
| 194 | GmaAffx.90570.1.S1_at | 0.381 | CF806295 | Unclassified - Hypothetical | | | |
| | | | | Protein Supported by cDNA | | | |
| 195 | GmaAffx.83377.2.S1_at | 0.38 | BM893101 | Transcription | Transcription | Basic Helix- | |
| | | | | | Factor | Loop-Helix | |
| | | | | | | (bHLH) | |
| 196 | Gma.7494.1.S1_at | 0.38 | BU548105 | Signal Transduction | Protein | | Serine/threonine protein phosphatase |
| | | | | | Phosphatase | | |
| 197 | GmaAffx.1301.68.S1_s_at | 0.378 | BI974374 | Intracellular Traffic | | | Annexin |
| 198 | GmaAffx.88786.1.A1_s_at | 0.376 | CK606552 | Cell Structure | | | O-methyltransferase |
| 199 | Gma.6550.3.S1_s_at | 0.374 | AW234450 | Metabolism | | | Hydroxyindole-O-methyltransferase and |
| | | | | | | | related SAM-dependent methyltransferases |
| 200 | GmaAffx.11999.1.S1_at | 0.374 | AW567661 | No Homology to Known | | | |
| | | | | Proteins | | | |
| 201 | GmaAffx.66451.1.S1_at | 0.373 | AW309198 | Secondary Metabolism | | | |
| 202 | GmaAffx.79888.1.S1_at | 0.372 | AI988123 | Signal Transduction | Protein Kinase | | |
| 203 | GmaAffx.80098.1.S1_at | 0.37 | BG042861 | Metabolism | | | Arylacetamide deacetylase |
| 204 | GmaAffx.1128.1.S1_at | 0.37 | AW348608 | Unclassified - Protein with | | | Copper chaperone |
| | | | | Unknown Function | | | |
| 205 | GmaAffx.90812.1.S1_s_at | 0.37 | CF806537 | Disease & Defense | | | |
| 206 | GmaAffx.9926.2.S1_at | 0.368 | BF597587 | Unclassified - Hypothetical | | | |
| | | | | Protein Supported by cDNA | | | |
| 207 | Gma.6640.1.S1_at | 0.367 | BQ628278 | Disease & Defense | | | |
| 208 | GmaAffx.1301.59.S1_at | 0.367 | BI974591 | Intracellular Traffic | | | Annexin |
| 209 | Gma.1195.1.S1_s_at | 0.366 | AI442801 | Metabolism | | | Predicted flavoprotein |
| 210 | GmaAffx.91205.1.S1_at | 0.365 | CF807869 | Metabolism | | | Glyoxylate/hydroxypyruvate reductase (D- |
| | | | | | | | isomer-specific 2-hydroxy acid |
| | | | | | | | dehydrogenase superfamily) |
| 211 | GmaAffx.74899.1.S1_at | 0.364 | CA852377 | No Homology to Known | | | |
| | | | | Proteins | | | |
| 212 | GmaAffx.15613.1.S1_at | 0.364 | AI939073 | Unclassified - Hypothetical | | | |
| | | | | Protein NOT Supported by | | | |
| | | | | cDNA | | | |
| 213 | GmaAffx.18953.1.S1_at | 0.363 | BM178798 | No Homology to Known | | | |
| | | | | Proteins | | | |
| 214 | GmaAffx.46837.1.S1_at | 0.362 | BU550919 | Secondary Metabolism | | | Multicopper oxidases |
| 215 | GmaAffx.50646.1.A1_at | 0.362 | AW310451 | Unclassified - Hypothetical | | | Molybdenum cofactor sulfurase |
| | | | | Protein Supported by cDNA | | | |
| 216 | Gma.17835.1.S1_at | 0.361 | BE822840 | Metabolism | | | |
| 217 | Gma.6719.2.S1_at | 0.361 | BG726773 | No Homology to Known | | | |
| | | | | Proteins | | | |
| 218 | GmaAffx.86927.1.S1_at | 0.36 | CA937162 | No Homology to Known | | | |
| | | | | Proteins | | | |
| 219 | GmaAffx.93189.1.S1_s_at | 0.359 | CF808914 | Metabolism | | | Carbon-nitrogen hydrolase |
| 220 | Gma.821.1.S1_at | 0.357 | AF243371.1 | Metabolism | | | Glutathione S-transferase |
| 221 | GmaAffx.18599.1.S1_at | 0.357 | BI787464 | Secondary Metabolism | Amines | | Amine oxidase |
| | | | | | Biosynthesis | | |
| 222 | Gma.5634.1.S1_at | 0.356 | BE806891 | Secondary Metabolism | | | Steroid reductase |
| 223 | Gma.73.1.S1_at | 0.354 | AF098662.1 | Protein Destination & Storage | | | |
| 224 | GmaAffx.91960.1.S1_at | 0.353 | CF807685 | Metabolism | | | Branched chain alpha-keto acid |
| | | | | | | | dehydrogenase complex, alpha subunit |
| 225 | GmaAffx.87793.1.S1_at | 0.353 | BI941782 | Unclassified - Hypothetical | | | |
| | | | | Protein NOT Supported by | | | |
| | | | | cDNA | | | |
| 226 | GmaAffx.6790.1.S1_at | 0.352 | AI856683 | Disease & Defense | | | Soluble epoxide hydrolase |
| 227 | GmaAffx.92192.1.S1_s_at | 0.352 | CF806406 | Disease & Defense | | | |
| 228 | Gma.17739.1.S1_at | 0.352 | AW310535 | Unclassified - Hypothetical | | | |
| | | | | Protein Supported by cDNA | | | |
| 229 | Gma.7120.2.S1_a_at | 0.35 | BG041285 | Metabolism | | | Alanine-glyoxylate aminotransferase AGT2 |
| 230 | GmaAffx.478.1.S1_at | 0.348 | BE822248 | Transcription | Transcription | Homeodomain | Transcription factor MEIS1 and related HOX |
| | | | | | Factor | | domain proteins |
| 231 | GmaAffx.11892.1.S1_at | 0.348 | BE820605 | Secondary Metabolism | | | Iron/ascorbate family oxidoreductases |
| 232 | GmaAffx.82218.1.S1_at | 0.347 | BI702052 | Secondary Metabolism | | | Iron/ascorbate family oxidoreductases |
| 233 | GmaAffx.93642.1.S1_s_at | 0.347 | CF809367 | Disease & Defense | | | |
| 234 | Gma.6047.1.S1_s_at | 0.346 | BQ611159 | Unclassified - Protein with | | | |
| | | | | Unknown Function | | | |
| 235 | Gma.5634.1.S1_s_at | 0.344 | BE806891 | Secondary Metabolism | | | Steroid reductase |
| 236 | Gma.18006.1.S1_at | 0.343 | CF922923 | Transcription | Transcription | Homeodomain | Transcription factor MEIS1 and related HOX |
| | | | | | Factor | | domain proteins |
| 237 | GmaAffx.62195.1.S1_at | 0.343 | AW472439 | No Homology to Known | | | |
| | | | | Proteins | | | |
| 238 | Gma.10854.1.S1_at | 0.343 | CA802334 | No Homology to Known | | | |
| | | | | Proteins | | | |
| 239 | GmaAffx.30593.1.S1_at | 0.342 | BE803365 | Unclassified - Hypothetical | | | |
| | | | | Protein Supported by cDNA | | | |
| 240 | Gma.5258.1.S1_at | 0.341 | BE658899 | No Homology to Known | | | |
| | | | | Proteins | | | |
| 241 | Gma.4401.2.S1_a_at | 0.339 | BI970725 | Disease & Defense | | | |
| 242 | GmaAffx.75740.1.S1_at | 0.338 | BG157373 | Unclassified - Hypothetical | | | |
| | | | | Protein NOT Supported by | | | |
| | | | | cDNA | | | |
| 243 | GmaAffx.64261.1.A1_at | 0.338 | BI969080 | Unclassified - Protein with | | | 1,4-benzoquinone reductase-like; Trp |
| | | | | Unknown Function | | | repressor binding protein-like/protoplast- |
| | | | | | | | secreted protein |
| 244 | Gma.4981.1.A1_at | 0.337 | BI970256 | No Homology to Known | | | |
| | | | | Proteins | | | |
| 245 | Gma.7254.1.S1_at | 0.332 | BU546166 | Unclassified - Hypothetical | | | |
| | | | | Protein Supported by cDNA | | | |
| 246 | GmaAffx.91312.1.S1_s_at | 0.332 | CF806002 | Transcription | Transcription | Homeodomain | Transcription factor HEX, contains HOX and |
| | | | | | Factor | | HALZ domains |
| 247 | Gma.14075.1.S1_at | 0.331 | CD415333 | Protein Destination & Storage | Storage Protein | | Ca2+-independent phospholipase A2 |
| 248 | Gma.15155.2.S1_at | 0.33 | BG789619 | Unclassified - Hypothetical | | | |
| | | | | Protein Supported by cDNA | | | |
| 249 | GmaAffx.61872.1.S1_at | 0.329 | AW102386 | Signal Transduction | Protein Kinase | | |
| 250 | GmaAffx.91185.1.S1_at | 0.327 | CF806910 | Cell Structure | | | Pectin acetylesterase and similar proteins |
| 251 | GmaAffx.47230.1.S1_at | 0.327 | CA820051 | Transporter | | | Amino acid transporters |
| 252 | Gma.5622.1.S1_at | 0.326 | BG363493 | No Homology to Known | | | |
| | | | | Proteins | | | |
| 253 | GmaAffx.26859.1.S1_s_at | 0.325 | BM085620 | Disease & Defense | | | Glutathione S-transferase |
| 254 | Gma.10342.1.S1_at | 0.323 | BE058741 | Unclassified - Hypothetical | | | |
| | | | | Protein Supported by cDNA | | | |
| 255 | Gma.15605.1.S1_at | 0.321 | BM143798 | Transcription | Transcription | AP2/EREBP | |
| | | | | | Factor | | |
| 256 | GmaAffx.56301.1.S1_at | 0.32 | BQ610805 | No Homology to Known | | | |
| | | | | Proteins | | | |
| 257 | Gma.6046.1.S1_at | 0.32 | BE022758 | Unclassified - Hypothetical | | | |
| | | | | Protein NOT Supported by | | | |
| | | | | cDNA | | | |
| 258 | Gma.6442.2.S1_at | 0.32 | BM270161 | Metabolism | | | Lysophospholipase |
| 259 | GmaAffx.92166.1.S1_s_at | 0.318 | CF806313 | Signal Transduction | Unclassified | | Aspartyl protease |
| 260 | GmaAffx.3711.1.A1_at | 0.318 | BE822116 | Unclassified - Hypothetical | | | |
| | | | | Protein NOT Supported by | | | |
| | | | | cDNA | | | |
| 261 | Gma.3745.1.S1_at | 0.318 | AW156339 | Secondary Metabolism | Amines | | Amine oxidase |
| | | | | | Biosynthesis | | |
| 262 | GmaAffx.21924.1.S1_at | 0.316 | BQ611867 | Disease & Defense | | | |
| 263 | GmaAffx.37615.1.S1_at | 0.315 | BQ454150 | No Homology to Known | | | |
| | | | | Proteins | | | |
| 264 | GmaAffx.89772.10.A1_s_at | 0.315 | CK606097 | Disease & Defense | | | |
| 265 | Gma.5215.1.S1_at | 0.313 | AI988141 | Unclassified - Hypothetical | | | BCL2-associated athanogene-like proteins |
| | | | | Protein Supported by cDNA | | | and related BAG family chaperone regulators |
| 266 | Gma.6623.1.S1_at | 0.313 | BI702268 | Unclassified - Protein with | | | 1,4-benzoquinone reductase-like; Trp |
| | | | | Unknown Function | | | repressor binding protein-like/protoplast- |
| | | | | | | | secreted protein |
| 267 | GmaAffx.74573.1.S1_at | 0.311 | BU763133 | No Homology to Known | | | |
| | | | | Proteins | | | |
| 268 | GmaAffx.21978.1.A1_at | 0.311 | CA803009 | No Homology to Known | | | |
| | | | | Proteins | | | |
| 269 | GmaAffx.76346.1.A1_at | 0.311 | BQ612378 | Energy | | | 1,4-benzoquinone reductase-like; Trp |
| | | | | | | | repressor binding protein-like/protoplast- |
| | | | | | | | secreted protein |
| 270 | Gma.1137.1.S1_x_at | 0.31 | BF067710 | Metabolism | | | |
| 271 | GmaAffx.77269.1.S1_at | 0.308 | BM093698 | No Homology to Known | | | |
| | | | | Proteins | | | |
| 272 | Gma.15568.1.S1_at | 0.305 | AI442650 | Disease & Defense | | | |
| 273 | Gma.17686.1.S1_at | 0.304 | BF597378 | Metabolism | Cytochrome | CYP76C | Cytochrome P450 CYP2 subfamily |
| | | | | | P450 | | |
| 274 | Gma.15469.1.S1_at | 0.302 | BU546655 | Cell Structure | | | Alpha tubulin |
| 275 | Gma.15155.2.S1_a_at | 0.302 | BG789619 | Unclassified - Hypothetical | | | |
| | | | | Protein Supported by cDNA | | | |
| 276 | GmaAffx.57748.1.S1_at | 0.302 | AW306274 | No Homology to Known | | | |
| | | | | Proteins | | | |
| 277 | Gma.3976.1.S1_at | 0.301 | BI972268 | No Homology to Known | | | |
| | | | | Proteins | | | |
| 278 | Gma.15155.1.S1_a_at | 0.301 | BF423817 | Unclassified - Hypothetical | | | |
| | | | | Protein Supported by cDNA | | | |
| 279 | GmaAffx.41078.1.S1_at | 0.3 | BI317149 | Metabolism | | | Alcohol dehydrogenase, class III |
| 280 | Gma.4093.2.S1_at | 0.3 | BQ081460 | Transporter | | | |
| 281 | GmaAffx.62509.2.S1_at | 0.299 | BE021475 | Transcription | Transcription | MYB | Transcription factor, Myb superfamily |
| | | | | | Factor | | |
| 282 | GmaAffx.37055.1.A1_at | 0.299 | BM142939 | Metabolism | | | UDP-glucuronosyl and UDP-glucosyl |
| | | | | | | | transferase |
| 283 | GmaAffx.68724.1.A1_at | 0.299 | BI970438 | No Homology to Known | | | |
| | | | | Proteins | | | |
| 284 | Gma.5867.1.A1_s_at | 0.297 | BI316163 | Metabolism | | | |
| 285 | Gma.752.1.A1_at | 0.297 | AW432463 | Transcription | Transcription | Basic Helix- | |
| | | | | | Factor | Loop-Helix | |
| | | | | | | (bHLH) | |
| 286 | GmaAffx.9926.1.A1_at | 0.296 | CD411291 | No Homology to Known | | | |
| | | | | Proteins | | | |
| 287 | Gma.4249.1.S1_at | 0.296 | BF324893 | Secondary Metabolism | | | Methyltransferase |
| 288 | Gma.7454.1.S1_a_at | 0.296 | CD405901 | Transcription | Transcription | AUX/IAA | |
| | | | | | Factor | | |
| 289 | Gma.7120.1.S1_at | 0.293 | BM093481 | Metabolism | | | Alanine-glyoxylate aminotransferase AGT2 |
| 290 | Gma.1716.1.S1_at | 0.292 | AW156801 | Unclassified - Hypothetical | | | |
| | | | | Protein Supported by cDNA | | | |
| 291 | GmaAffx.4968.1.S1_at | 0.289 | BI967691 | Unclassified - Hypothetical | | | |
| | | | | Protein Supported by cDNA | | | |
| 292 | Gma.10559.1.S1_s_at | 0.289 | BU544359 | No Homology to Known | | | |
| | | | | Proteins | | | |
| 293 | GmaAffx.16986.2.S1_at | 0.287 | AW277368 | Metabolism | | | Hydroxyindole-O-methyltransferase and |
| | | | | | | | related SAM-dependent methyltransferases |
| 294 | GmaAffx.24102.1.S1_at | 0.286 | BQ453911 | Unclassified - Hypothetical | | | |
| | | | | Protein Supported by cDNA | | | |
| 295 | Gma.17921.2.S1_s_at | 0.284 | BM085020 | Metabolism | Sucrose | | Glycosyltransferase |
| | | | | | Biosynthesis | | |
| 296 | Gma.5863.2.S1_s_at | 0.283 | AW350762 | Secondary Metabolism | | | |
| 297 | GmaAffx.56715.1.S1_at | 0.283 | BG041616 | Disease & Defense | | | |
| 298 | Gma.364.1.A1_at | 0.283 | AW309875 | Unclassified - Hypothetical | | | |
| | | | | Protein Supported by cDNA | | | |
| 299 | GmaAffx.90970.1.S1_s_at | 0.28 | CF806695 | Unclassified - Hypothetical | | | |
| | | | | Protein Supported by cDNA | | | |
| 300 | GmaAffx.13912.1.S1_at | 0.28 | BI699258 | Disease & Defense | | | Purple (tartrate-resistant) acid phosphatase |
| 301 | GmaAffx.57469.1.S1_at | 0.274 | AW351054 | Unclassified - Hypothetical | | | |
| | | | | Protein Supported by cDNA | | | |
| 302 | Gma.2842.1.S1_at | 0.274 | BG511652 | Unclassified - Protein with | | | Predicted Yippee-type zinc-binding protein |
| | | | | Unknown Function | | | |
| 303 | GmaAffx.86745.1.S1_s_at | 0.271 | BI424960 | Disease & Defense | | | |
| 304 | Gma.6599.1.S1_at | 0.271 | BQ629084 | Cell Structure | | | |
| 305 | Gma.382.1.A1_at | 0.268 | AW349068 | Metabolism | | | Arylacetamide deacetylase |
| 306 | GmaAffx.91555.1.A1_s_at | 0.268 | CF807280 | Metabolism | | | |
| 307 | Gma.3604.1.S1_at | 0.267 | AW349604 | Cell Structure | | | O-methyltransferase |
| 308 | Gma.17840.1.S1_at | 0.267 | BE821995 | No Homology to Known | | | |
| | | | | Proteins | | | |
| 309 | Gma.9086.1.S1_at | 0.263 | AW184932 | Cell Growth & Division | | | |
| 310 | GmaAffx.71842.1.S1_at | 0.263 | BE555317 | Metabolism | | | Possible pfkB family carbohydrate kinase |
| 311 | GmaAffx.62509.1.S1_s_at | 0.261 | BE022332 | Transcription | Transcription | MYB | Transcription factor, Myb superfamily |
| | | | | | Factor | | |
| 312 | GmaAffx.90104.1.S1_s_at | 0.26 | CF805829 | Transporter | | | Permease of the major facilitator superfamily |
| 313 | Gma.2414.1.S1_at | 0.259 | BU551192 | Protein Destination & Storage | | | |
| 314 | GmaAffx.84751.1.A1_at | 0.257 | AW349702 | Transcription | Transcription | MYB | Transcription factor, Myb superfamily |
| | | | | | Factor | | |
| 315 | Gma.6999.2.S1_s_at | 0.249 | X60043.1 | Disease & Defense | | | |
| 316 | Gma.4222.1.S1_at | 0.248 | BU083971 | Unclassified - Hypothetical | | | Arylacetamide deacetylase |
| | | | | Protein NOT Supported by | | | |
| | | | | cDNA | | | |
| 317 | GmaAffx.51409.1.S1_at | 0.247 | BM309035 | Transporter | | | Synaptic vesicle transporter SVOP and |
| | | | | | | | related transporters (major facilitator |
| | | | | | | | superfamily) |
| 318 | Gma.3987.1.S1_at | 0.247 | BQ453408 | Unclassified - Hypothetical | | | Sodium sulfate symporter and related arsenite |
| | | | | Protein NOT Supported by | | | permeases |
| | | | | cDNA | | | |
| 319 | Gma.9828.1.S1_at | 0.245 | AF529301.1 | Metabolism | | | Haloacid dehalogenase-like hydrolase |
| 320 | GmaAffx.89259.1.S1_s_at | 0.245 | CK605646 | Metabolism | | | |
| 321 | GmaAffx.70008.1.S1_at | 0.244 | BU762337 | Transcription | Transcription | MYB | Transcription factor, Myb superfamily |
| | | | | | Factor | | |
| 322 | Gma.6679.1.A1_at | 0.241 | BU544081 | Transcription | Transcription | MYB | Transcription factor, Myb superfamily |
| | | | | | Factor | | |
| 323 | Gma.7422.1.S1_at | 0.24 | BQ612005 | Transporter | | | |
| 324 | Gma.1137.1.S1_a_at | 0.239 | BF067710 | Metabolism | | | |
| 325 | GmaAffx.41917.1.S1_at | 0.238 | BE658926 | Disease & Defense | | | |
| 326 | Gma.3161.2.S1_at | 0.235 | CD400025 | Secondary Metabolism | | | Iron/ascorbate family oxidoreductases |
| 327 | GmaAffx.83818.1.S1_at | 0.235 | BU762716 | No Homology to Known | | | |
| | | | | Proteins | | | |
| 328 | Gma.4796.1.A1_at | 0.234 | AW102323 | Unclassified - Hypothetical | | | Uncharacterized conserved protein |
| | | | | Protein NOT Supported by | | | |
| | | | | cDNA | | | |
| 329 | GmaAffx.15119.1.A1_at | 0.234 | BU544528 | Metabolism | | | Possible pfkB family carbohydrate kinase |
| 330 | GmaAffx.87433.1.S1_at | 0.234 | BI701509 | Unclassified - Hypothetical | | | |
| | | | | Protein Supported by cDNA | | | |
| 331 | GmaAffx.74323.1.S1_at | 0.233 | BI426587 | Unclassified - Hypothetical | | | Endoplasmic reticulum protein EP58, |
| | | | | Protein NOT Supported by | | | contains filamin rod domain and KDEL motif |
| | | | | cDNA | | | |
| 332 | Gma.4674.1.A1_at | 0.231 | AW309836 | Metabolism | | | Arylacetamide deacetylase |
| 333 | GmaAffx.47739.1.S1_at | 0.228 | BM522736 | Unclassified - Protein with | | | |
| | | | | Unknown Function | | | |
| 334 | Gma.18006.1.A1_at | 0.227 | AI496486 | Transcription | Transcription | Homeodomain | Transcription factor MEIS1 and related HOX |
| | | | | | Factor | | domain proteins |
| 335 | Gma.6999.1.S1_x_at | 0.225 | AF529303.1 | Disease & Defense | | | |
| 336 | Gma.10559.1.S1_at | 0.221 | BU544359 | No Homology to Known | | | |
| | | | | Proteins | | | |
| 337 | GmaAffx.45334.1.S1_at | 0.217 | BI969317 | Secondary Metabolism | GA | | Iron/ascorbate family oxidoreductases |
| | | | | | Biosynthesis | | |
| 338 | GmaAffx.2026.1.A1_at | 0.216 | BU550139 | Transcription | Transcription | NAC Domain | |
| | | | | | Factor | | |
| 339 | Gma.3175.1.S1_at | 0.216 | AI736154 | Unclassified - Hypothetical | | | |
| | | | | Protein NOT Supported by | | | |
| | | | | cDNA | | | |
| 340 | Gma.7549.1.S1_at | 0.21 | BG508637 | Unclassified - Protein with | | | |
| | | | | Unknown Function | | | |
| 341 | GmaAffx.89772.14.A1_s_at | 0.209 | CK606614 | Disease & Defense | | | |
| 342 | Gma.5611.1.S1_at | 0.208 | AW309826 | Metabolism | Cytochrome | CYP81D | Cytochrome P450 CYP2 subfamily |
| | | | | | P450 | | |
| 343 | GmaAffx.24521.1.S1_at | 0.207 | BU761767 | Unclassified - Hypothetical | | | |
| | | | | Protein NOT Supported by | | | |
| | | | | cDNA | | | |
| 344 | GmaAffx.14030.1.S1_at | 0.204 | BE021503 | No Homology to Known | | | |
| | | | | Proteins | | | |
| 345 | Gma.18052.1.S1_at | 0.204 | BF595464 | Transporter | | | |
| 346 | GmaAffx.59470.1.S1_at | 0.202 | BI469872 | Secondary Metabolism | | | Phenylalanine and histidine ammonia-lyase |
| 347 | Gma.3165.1.S1_at | 0.202 | CD406755 | Transcription | Transcription | Homeodomain | Transcription factor HEX, contains HOX and |
| | | | | | Factor | | HALZ domains |
| 348 | GmaAffx.61593.1.S1_at | 0.197 | BU765478 | Unclassified - Hypothetical | | | Sodium sulfate symporter and related arsenite |
| | | | | Protein NOT Supported by | | | permeases |
| | | | | cDNA | | | |
| 349 | Gma.9638.1.A1_at | 0.195 | CA936403 | Disease & Defense | | | |
| 350 | GmaAffx.81618.1.S1_at | 0.194 | AW706403 | No Homology to Known | | | |
| | | | | Proteins | | | |
| 351 | Gma.1269.1.S1_at | 0.192 | AW309671 | Metabolism | | | Flavonol reductase/cinnamoyl-CoA reductase |
| 352 | Gma.6081.1.S1_at | 0.187 | BI967474 | Disease & Defense | | | Predicted chitinase |
| 353 | Gma.17877.1.S1_at | 0.185 | BQ612471 | Unclassified - Protein with | | | |
| | | | | Unknown Function | | | |
| 354 | Gma.15996.1.S1_x_at | 0.184 | BQ454193 | Unclassified - Hypothetical | | | |
| | | | | Protein Supported by cDNA | | | |
| 355 | Gma.6075.1.S1_at | 0.184 | AW317183 | Disease & Defense | | | |
| 356 | Gma.16947.1.S1_at | 0.184 | BG155375 | Unclassified - Hypothetical | | | |
| | | | | Protein Supported by cDNA | | | |
| 357 | GmaAffx.16986.1.A1_s_at | 0.182 | AW351116 | Metabolism | | | Hydroxyindole-O-methyltransferase and |
| | | | | | | | related SAM-dependent methyltransferases |
| 358 | Gma.6643.2.S1_at | 0.177 | BE023121 | Secondary Metabolism | | | Sulfotransferase |
| 359 | Gma.6999.1.S1_s_at | 0.174 | AF529303.1 | Disease & Defense | | | |
| 360 | GmaAffx.5531.1.S1_at | 0.167 | BQ740711 | Cell Structure | | | |
| 361 | Gma.6999.3.S1_s_at | 0.162 | CF921432 | Disease & Defense | | | |
| 362 | Gma.5309.1.S1_at | 0.158 | BM092263 | Unclassified - Hypothetical | | | |
| | | | | Protein NOT Supported by | | | |
| | | | | cDNA | | | |
| 363 | GmaAffx.70959.1.S1_at | 0.157 | BE611219 | Unclassified - Hypothetical | | | Glucose-6-phosphate/phosphate and |
| | | | | Protein NOT Supported by | | | phosphoenolpyruvate/phosphate antiporter |
| | | | | cDNA | | | |
| 364 | GmaAffx.19467.1.S1_at | 0.155 | BQ453237 | Signal Transduction | Protein Kinase | | Serine/threonine protein kinase |
| 365 | Gma.9010.1.S1_at | 0.153 | CF921194 | Disease & Defense | | | |
| 366 | Gma.4873.1.S1_at | 0.148 | BI969532 | Signal Transduction | Protein Kinase | | Serine/threonine protein kinase |
| 367 | Gma.15036.2.A1_at | 0.148 | CD411972 | Unclassified - Protein with | | | Predicted membrane protein |
| | | | | Unknown Function | | | |
| 368 | Gma.17662.2.S1_s_at | 0.146 | BQ630190 | Unclassified - Hypothetical | | | |
| | | | | Protein NOT Supported by | | | |
| | | | | cDNA | | | |
| 369 | GmaAffx.9944.1.S1_at | 0.146 | BM567882 | Cell Structure | | | |
| 370 | Gma.10216.1.S1_at | 0.145 | BI969369 | Metabolism | | | Hydroxyindole-O-methyltransferase and |
| | | | | | | | related SAM-dependent methyltransferases |
| 371 | GmaAffx.51464.1.S1_at | 0.142 | BQ628397 | No Homology to Known | | | |
| | | | | Proteins | | | |
| 372 | Gma.336.1.A1_at | 0.142 | AW310815 | Metabolism | Sulfur | | |
| | | | | | Metabolism | | |
| 373 | GmaAffx.41230.1.A1_at | 0.135 | BI471609 | Unclassified - Hypothetical | | | |
| | | | | Protein NOT Supported by | | | |
| | | | | cDNA | | | |
| 374 | Gma.4260.1.S1_at | 0.134 | BM177256 | Unclassified - Hypothetical | | | |
| | | | | Protein NOT Supported by | | | |
| | | | | cDNA | | | |
| 375 | Gma.3810.1.S1_s_at | 0.125 | BI972063 | Disease & Defense | | | |
| 376 | Gma.6462.2.S1_a_at | 0.119 | BE555263 | Unclassified - Hypothetical | | | |
| | | | | Protein NOT Supported by | | | |
| | | | | cDNA | | | |
| 377 | GmaAffx.65393.1.S1_s_at | 0.119 | BU760770 | No Homology to Known | | | |
| | | | | Proteins | | | |
| 378 | GmaAffx.92479.1.S1_s_at | 0.112 | CF808204 | No Homology to Known | | | |
| | | | | Proteins | | | |
| 379 | Gma.17446.1.A1_at | 0.106 | AI461103 | Unclassified - Protein with | | | |
| | | | | Unknown Function | | | |
| 380 | Gma.17890.1.S1_at | 0.101 | AW432965 | No Homology to Known | | | |
| | | | | Proteins | | | |
| 381 | GmaAffx.81750.1.S1_at | 0.0985 | AW306336 | No Homology to Known | | | |
| | | | | Proteins | | | |
| 382 | Gma.2133.1.S1_at | 0.0855 | AW310071 | Unclassified - Protein with | | | |
| | | | | Unknown Function | | | |
| 383 | Gma.15625.1.S1_x_at | 0.0841 | U77678.1 | Metabolism | Amino Acid | | Asparagine synthase (glutamine-hydrolyzing) |
| | | | | | Biosynthesis/Metabolism | | |
| 384 | Gma.15625.1.S1_a_at | 0.0771 | U77678.1 | Metabolism | Amino Acid | | Asparagine synthase (glutamine-hydrolyzing) |
| | | | | | Biosynthesis/Metabolism | | |
| 385 | Gma.11211.1.S1_s_at | 0.074 | AI442578 | No Homology to Known | | | |
| | | | | Proteins | | | |
| 386 | Gma.8365.1.S1_at | 0.07 | CD396813 | Unclassified - Hypothetical | | | |
| | | | | Protein NOT Supported by | | | |
| | | | | cDNA | | | |
| 387 | Gma.1332.1.S1_x_at | 0.0609 | BI970666 | Secondary Metabolism | Ethylene | | 1-aminocyclopropane-1-carboxylate |
| | | | | | Biosynthesis | | synthase, and related proteins |
| 388 | Gma.8425.1.S1_s_at | 0.0562 | D83968.1 | Metabolism | Cytochrome | CYP93D | Cytochrome P450 CYP2 subfamily |
| | | | | | P450 | | |
| 389 | Gma.79.1.S1_x_at | 0.0493 | S44202.1 | Unclassified - Protein with | | | |
| | | | | Unknown Function | | | |
| 390 | GmaAffx.19541.1.S1_s_at | 0.044 | BQ612990 | No Homology to Known | | | |
| | | | | Proteins | | | |
| 391 | Gma.5947.1.S1_s_at | 0.0413 | BQ610509 | Disease & Defense | | | |
| 392 | Gma.79.1.S1_s_at | 0.0381 | S44202.1 | Unclassified - Protein with | | | |
| | | | | Unknown Function | | | |

TABLE 11

| ID | Gene Name | Fold Change | Soybean_GenBank_Acc | Functional Category | Sub-Family | Gene Family | Additional Description |
|---|---|---|---|---|---|---|---|
| 1 | Gma.4058.1.S1_at | 22.22 | AW310338 | Transporter | | | Predicted transporter (major facilitator superfamily) |
| 2 | GmaAffx.32345.1.A1_s_at | 16.29 | BI970277 | Cell Structure | | | |
| 3 | Gma.5716.1.S1_at | 11.64 | BQ453925 | No Homology to Known | | | |
| | | | | Proteins | | | |
| 4 | GmaAffx.87679.1.S1_at | 9.533 | BM523734 | Metabolism | Trehalose | | Trehalose-6-phosphate synthase component |
| | | | | | Biosynthesis | | TPS1 and related subunits |
| 5 | GmaAffx.89567.1.A1_at | 9.001 | CK606157 | Signal Transduction | Calcium | Calcium | Ca2+/calmodulin-dependent protein kinase, |
| | | | | | Binding | Dependent | EF-Hand protein superfamily |
| | | | | | | Protein Kinase | |
| 6 | GmaAffx.19116.1.S1_at | 7.985 | BI968419 | Signal Transduction | Protein Kinase | | |
| 7 | GmaAffx.87679.2.S1_s_at | 7.575 | AW102083 | Metabolism | Trehalose | | Trehalose-6-phosphate synthase component |
| | | | | | Biosynthesis | | TPS1 and related subunits |
| 8 | Gma.3804.1.A1_at | 7.423 | AW310249 | Disease & Defense | | | |
| 9 | GmaAffx.9866.1.S1_at | 7.144 | BM524432 | Metabolism | | | |
| 10 | GmaAffx.9618.1.S1_at | 7.08 | BM092432 | No Homology to Known | | | |
| | | | | Proteins | | | |
| 11 | Gma.8416.1.S1_at | 6.333 | U13987.1 | Metabolism | | | Cytochrome b5 |
| 11 | Gma.8416.1.S1_at | 6.333 | U13987.1 | Metabolism | | | NADH-cytochrome b-5 reductase |
| 11 | Gma.8416.1.S1_at | 6.333 | U13987.1 | Metabolism | | | Sulfite oxidase, molybdopterin-binding |
| | | | | | | | component |
| 12 | GmaAffx.89396.2.A1_s_at | 6.061 | CK605844 | Cell Structure | | | |
| 13 | GmaAffx.70875.1.S1_s_at | 6.049 | AW100693 | No Homology to Known | | | |
| | | | | Proteins | | | |
| 14 | GmaAffx.40860.2.S1_at | 5.75 | BG509282 | Disease & Defense | | | |
| 15 | Gma.10921.1.S1_s_at | 5.722 | BQ611250 | No Homology to Known | | | |
| | | | | Proteins | | | |
| 16 | Gma.152.1.S1_at | 5.694 | Y10492.1 | Metabolism | Cytochrome | CYP93D | Cytochrome P450 CYP2 subfamily |
| | | | | | P450 | | |
| 17 | Gma.17961.1.S1_at | 5.671 | AW350363 | Protein Destination & | Storage | | |
| | | | | Storage | Protein | | |
| 18 | GmaAffx.93541.2.S1_at | 5.572 | CF805889 | Protein Destination & | | | Gelatinase A and related matrix |
| | | | | Storage | | | metalloproteases |
| 19 | Gma.16425.2.S1_s_at | 5.528 | AW099501 | Unclassified - | | | Copper chaperone |
| | | | | Hypothetical Protein | | | |
| | | | | NOT Supported by cDNA | | | |
| 20 | Gma.11259.1.S1_at | 5.423 | AI442316 | Unclassified - | | | |
| | | | | Hypothetical Protein | | | |
| | | | | Supported by cDNA | | | |
| 21 | Gma.3888.2.S1_at | 5.349 | BM139532 | Unclassified - | | | Aldehyde reductase |
| | | | | Hypothetical Protein | | | |
| | | | | NOT Supported by cDNA | | | |
| 22 | GmaAffx.2743.1.S1_s_at | 5.137 | BM568087 | Signal Transduction | Protein Kinase | | |
| 23 | Gma.5629.1.S1_at | 5.076 | BQ740550 | Disease & Defense | | | |
| 24 | GmaAffic.81930.1.S1_at | 5.036 | BG789998 | Unclassified - | | | |
| | | | | Hypothetical Protein | | | |
| | | | | Supported by cDNA | | | |
| 25 | GmaAffx.2743.1.S1_at | 5.014 | BM568087 | Signal Transduction | Protein Kinase | | |
| 26 | Gma.5483.1.S1_s_at | 4.957 | CD414581 | Transcription | Transcription | Basic Leucine | |
| | | | | | Factor | Zipper (bZIP) | |
| 27 | Gma.4701.2.A1_a_at | 4.942 | BM526048 | Energy | | | Thioredoxin |
| 28 | Gma.4226.2.S1_at | 4.938 | BM568170 | Unclassified - | | | |
| | | | | Hypothetical Protein | | | |
| | | | | Supported by cDNA | | | |
| 29 | Gma.3639.1.S1_at | 4.902 | AB083030.1 | Protein Destination & | Storage | | |
| | | | | Storage | Protein | | |
| 30 | GmaAffx.70509.1.A1_at | 4.887 | CD400633 | No Homology to Known | | | |
| | | | | Proteins | | | |
| 31 | Gma.1400.2.S1_at | 4.811 | BG510739 | Signal Transduction | Protein Kinase | AtCIPKs | Serine/threonine protein kinase |
| 32 | GmaAffx.93260.1.S1_at | 4.807 | CF808985 | Signal Transduction | Protein Kinase | | |
| 33 | Gma.4071.1.S1_at | 4.771 | BG651692 | Unclassified - | | | |
| | | | | Hypothetical Protein | | | |
| | | | | Supported by cDNA | | | |
| 34 | GmaAffx.69888.1.A1_at | 4.769 | BU549275 | No Homology to Known | | | |
| | | | | Proteins | | | |
| 35 | Gma.3579.1.S1_at | 4.768 | AW349799 | Unclassified - | | | Kelch repeat-containing proteins |
| | | | | Hypothetical Protein | | | |
| | | | | Supported by cDNA | | | |
| 36 | GmaAffx.87872.1.S1_at | 4.606 | BM885036 | Metabolism | | | CTP synthase (UTP-ammonia lyase) |
| 37 | GmaAffx.18846.1.S1_at | 4.583 | BM091991 | Signal Transduction | Small GTPase | | FOG: WD40 repeat |
| 38 | GmaAffx.89075.1.S1_s_at | 4.503 | CK606454 | No Homology to Known | | | |
| | | | | Proteins | | | |
| 39 | GmaAffx.43297.1.A1_at | 4.485 | AI442864 | Unclassified - | | | |
| | | | | Hypothetical Protein | | | |
| | | | | NOT Supported by cDNA | | | |
| 40 | Gma.17605.1.S1_at | 4.441 | BI968208 | Secondary Metabolism | Flavonoid Biosynthesis | | |
| 41 | GmaAffx.89158.1.A1_at | 4.344 | CK605958 | Intracellular Traffic | | | Phosphatidylinositol transfer protein |
| | | | | | | | SEC14 and related proteins |
| 42 | GmaAffx.93462.1.S1_s_at | 4.271 | CF809187 | Signal Transduction | Protein Kinase | AtC1PKs | Serine/threonine protein kinase |
| 43 | Gma.16425.3.S1_s_at | 4.229 | AW508123 | Unclassified - Protein | | | Copper chaperone |
| | | | | with Unknown Function | | | |
| 44 | Gma.7474.1.A1_at | 4.206 | BU547968 | Unclassified - | | | |
| | | | | Hypothetical Protein | | | |
| | | | | Supported by cDNA | | | |
| 45 | Gma.16832.1.S1_s_at | 4.194 | L22029.1 | No Homology to Known | | | |
| | | | | Proteins | | | |
| 46 | GmaAffx.52494.1.S1_at | 4.174 | BE473800 | Unclassified - | | | |
| | | | | Hypothetical Protein | | | |
| | | | | NOT Supported by cDNA | | | |
| 47 | Gma.5689.3.S1_s_at | 4.168 | BE657645 | Protein Destination & | | | Gelatinase A and related matrix |
| | | | | Storage | | | metalloproteases |
| 48 | GmaAffx.62542.1.S1_at | 4.057 | BE659136 | Unclassified - | | | |
| | | | | Hypothetical Protein | | | |
| | | | | NOT Supported by cDNA | | | |
| 49 | GmaAffx.48172.1.S1_at | 4.033 | BF009291 | Transporter | | | Predicted small molecule transporter |
| 50 | GmaAffx.92537.1.S1_at | 4.019 | CF808262 | Energy | | | Thioredoxin |
| 51 | Gma.184.1.S1_at | 3.978 | AW101261 | Unclassified - | | | |
| | | | | Hypothetical Protein | | | |
| | | | | Supported by cDNA | | | |
| 52 | Gma.7726.1.A1_at | 3.929 | BM886413 | Unclassified - Protein | | | Ca2+-binding protein (centrin/caltractin), |
| | | | | with Unknown Function | | | EF-Hand superfamily protein |
| 53 | Gma.15889.1.S1_at | 3.928 | AW348133 | Transcription Factor | Transcription | WRKY | |
| 54 | Gma.2010.1.S1_a_at | 3.876 | CD402050 | Transcription Factor | Transcription | Zinc Finger | GATA-4/5/6 transcription factors |
| 55 | GmaAffx.55767.1.S1_at | 3.817 | BQ079515 | Signal Transduction | Calcium | | Calmodulin and related proteins (EF-Hand |
| | | | | | Binding | | superfamily) |
| 56 | GmaAffx.78720.2.S1_at | 3.816 | BI788325 | No Homology to Known | | | |
| | | | | Proteins | | | |
| 57 | Gma.6555.1.S1_at | 3.81 | BM954521 | Unclassified - | | | |
| | | | | Hypothetical Protein | | | |
| | | | | Supported by cDNA | | | |
| 58 | PsAffx.psHB044xM02f_at | 3.793 | | | | | |
| 59 | Gma.5631.1.S1_at | 3.748 | CD405214 | Transporter | | | Amino acid transporters |
| 60 | GmaAffx.24583.1.S1_at | 3.746 | BE609540 | Signal Transduction | Protein Kinase | | Serine/threonine protein kinase |
| 61 | Gma.2446.2.S1_at | 3.739 | BI427101 | Unclassified - | | | |
| | | | | Hypothetical Protein | | | |
| | | | | Supported by cDNA | | | |
| 62 | Gma.5816.1.S1_at | 3.726 | BI787790 | Unclassified - | | | Aldehyde reductase |
| | | | | Hypothetical Protein | | | |
| | | | | Supported by cDNA | | | |
| 63 | GmaAffx.93270.1.S1_x_at | 3.706 | CF808995 | No Homology to Known | | | |
| | | | | Proteins | | | |
| 64 | GmaAffx.78636.1.S1_at | 3.706 | BQ627652 | No Homology to Known | | | |
| | | | | Proteins | | | |
| 65 | GmaAffx.18649.1.S1_at | 3.693 | AW351025 | No Homology to Known | | | |
| | | | | Proteins | | | |
| 66 | GmaAffx.10340.1.S1_x_at | 3.668 | CA938421 | No Homology to Known | | | |
| | | | | Proteins | | | |
| 67 | Gma.13447.1.A1_at | 3.656 | CD398305 | No Homology to Known | | | |
| | | | | Proteins | | | |
| 68 | GmaAffx.9145.1.S1_s_at | 3.649 | BU763814 | Disease & Defense | | | |
| 69 | GmaAffx.65829.1.A1_at | 3.588 | CD392418 | Transcription | Transcription | AP2/EREBP | |
| | | | | | Factor | | |
| 70 | Gma.17789.1.S1_at | 3.562 | BG511704 | Unclassified - | | | |
| | | | | Hypothetical Protein | | | |
| | | | | Supported by cDNA | | | |
| 71 | Gma.12665.2.A1_at | 3.529 | CD394142 | Unclassified - | | | |
| | | | | Hypothetical Protein | | | |
| | | | | Supported by cDNA | | | |
| 72 | Gma.14471.2.S1_at | 3.523 | CD408292 | Transporter | | | Aquaporin (major intrinsic protein family) |
| 73 | Gma.15889.1.S1_s_at | 3.503 | AW348133 | Transcription | Transcription | WRKY | |
| | | | | | Factor | | |
| 74 | Gma.2488.1.S1_at | 3.467 | CD406079 | Protein Destination & | | | Serine carboxypeptidases (lysosomal |
| | | | | Storage | | | cathepsin A) |
| 75 | Gma.5579.1.S1_at | 3.467 | AW119360 | Secondary Metabolism | Flavonoid | | |
| | | | | | Biosynthesis | | |
| 76 | GmaAffx.36514.1.S1_at | 3.452 | BE658341 | Disease & Defense | | | |
| 77 | GmaAffx.8098.1.S1_at | 3.437 | AW156410 | No Homology to Known | | | |
| | | | | Proteins | | | |
| 78 | GmaAffx.89512.1.A1_at | 3.403 | CK606530 | No Homology to Known | | | |
| | | | | Proteins | | | |
| 79 | GmaAffx.47815.1.A1_at | 3.374 | CD415065 | Unclassified - | | | |
| | | | | Hypothetical Protein | | | |
| | | | | NOT Supported by cDNA | | | |
| 80 | Gma.8896.1.S1_at | 3.369 | BQ252812 | Transcription Factor | Transcription | AP2/EREBP | |
| 81 | GmaAffx.18566.1.A1_at | 3.363 | BU550417 | Unclassified - | | | |
| | | | | Hypothetical Protein | | | |
| | | | | NOT Supported by cDNA | | | |
| 82 | Gma.1221.1.S1_s_at | 3.359 | L23854.1 | Metabolism | | | Cytochrome b5 |
| 82 | Gma.1221.1.S1_s_at | 3.359 | L23854.1 | Metabolism | | | NADH-cytochrome b-5 reductase |
| 82 | Gma.1221.1.S1_s_at | 3.359 | L23854.1 | Metabolism | | | Sulfite oxidase, molybdopterin-binding |
| | | | | | | | component |
| 83 | GmaAffx.90960.1.A1_s_at | 3.356 | CF806685 | Signal Transduction | Protein Kinase | | |
| 84 | Gma.12665.1.S1_at | 3.331 | BE347113 | Unclassified - | | | |
| | | | | Hypothetical Protein | | | |
| | | | | Supported by cDNA | | | |
| 85 | Gma.340.1.S1_at | 3.327 | AW350127 | Cell Structure | | | |
| 86 | Gma.6266.1.A1_at | 3.296 | CD413418 | Unclassified - Protein | | | |
| | | | | with Unknown Function | | | |
| 87 | GmaAffx.85374.1.S1_at | 3.263 | BI317002 | Signal Transduction | Protein Kinase | | |
| 88 | Gma.4470.1.S1_s_at | 3.26 | AW156251 | Unclassified - Protein | | | |
| | | | | with Unknown Function | | | |
| 89 | GmaAffx.11276.1.S1_s_at | 3.252 | AW567677 | Unclassified - | | | |
| | | | | Hypothetical Protein | | | |
| | | | | Supported by cDNA | | | |
| 90 | GmaAffx.54265.1.S1_s_at | 3.24 | BU546096 | Signal Transduction | Protein Kinase | | |
| 91 | GmaAffx.37908.1.A1_at | 3.229 | BU546116 | Signal Transduction | Protein Kinase | | |
| 92 | Gma.12867.1.A1_at | 3.18 | CD393518 | Unclassified - | | | |
| | | | | Hypothetical Protein | | | |
| | | | | Supported by cDNA | | | |
| 93 | GmaAffx.10340.1.S1_at | 3.178 | CA938421 | No Homology to Known | | | |
| | | | | Proteins | | | |
| 94 | GmaAffx.78434.1.S1_at | 3.177 | BG041904 | Unclassified - | | | Predicted membrane protein, contains DoH |
| | | | | Hypothetical Protein | | | and Cytochrome b-561/ferric reductase |
| | | | | NOT Supported by cDNA | | | transmembrane domains |
| 95 | Gma.13433.1.A1_at | 3.173 | CD398049 | No Homology to Known | | | |
| | | | | Proteins | | | |
| 96 | Gma.5629.2.S1_a_at | 3.165 | BG042866 | Disease & Defense | | | |
| 97 | Gma.2972.1.S1_at | 3.158 | AW350602 | Unclassified - | | | Phosphatidylinositol transfer protein |
| | | | | Hypothetical Protein | | | SEC14 and related proteins |
| | | | | Supported by cDNA | | | |
| 98 | GmaAffx.56563.1.S1_at | 3.133 | CA820002 | No Homology to Known | | | |
| | | | | Proteins | | | |
| 99 | Gma.368.1.S1_at | 3.124 | AW349594 | No Homology to Known | | | |
| | | | | Proteins | | | |
| 100 | GmaAffx.67477.2.S1_at | 3.116 | AW509184 | Cell Structure | | | |
| 101 | Gma.10307.1.S1_at | 3.114 | CA936845 | Unclassified - | | | |
| | | | | Hypothetical Protein | | | |
| | | | | NOT Supported by cDNA | | | |
| 102 | Gma.16308.1.S1_s_at | 3.061 | U64916.1 | Unclassified - | | | Copper chaperone |
| | | | | Hypothetical Protein | | | |
| | | | | NOT Supported by cDNA | | | |
| 103 | GmaAffx.45530.1.S1_at | 3.048 | BG789917 | Signal Transduction | Protein Kinase | | |
| 104 | GmaAffx.11858.1.A1_s_at | 3.047 | AW351023 | Metabolism | Phospholipid | | Glycosylphosphatidylinositol-specific |
| | | | | | Biosynthesis | | phospholipase C |
| 105 | GmaAffx.90042.1.S1_at | 3.017 | CF805767 | Signal Transduction | Calcium | | Calmodulin and related proteins (EF-Hand |
| | | | | | Binding | | superfamily) |
| 106 | Gma.3777.1.S1_a_at | 3.008 | CD399954 | Unclassified - | | | |
| | | | | Hypothetical Protein | | | |
| | | | | Supported by cDNA | | | |
| 107 | GmaAffx.65419.1.S1_at | 3.003 | CD405955 | Signal Transduction | Protein Kinase | | |
| 108 | GmaAffx.40860.1.S1_at | 3.002 | BQ628787 | Disease & Defense | | | |
| 109 | Gma.8797.1.A1_at | 2.953 | BI969881 | Unclassified - | | | |
| | | | | Hypothetical Protein | | | |
| | | | | NOT Supported by cDNA | | | |
| 110 | GmaAffx.84266.1.S1_at | 2.952 | AI495798 | Signal Transduction | Protein Kinase | AtCIPKs | Serine/threonine protein kinase |
| 111 | GmaAffx.66979.1.S1_at | 2.944 | BI497616 | Signal Transduction | Protein Kinase | AtCIPKs | Serine/threonine protein kinase |
| 112 | GmaAffx.78720.1.S1_at | 2.93 | BM526650 | No Homology to Known | | | |
| | | | | Proteins | | | |
| 113 | GmaAffx.21211.1.S1_at | 2.919 | BU550606 | Protein Destination & | | | Molecular chaperone (HSP90 family) |
| | | | | Storage | | | |
| 114 | Gma.15495.1.A1_at | 2.918 | CD409942 | Unclassified - | | | Serine/threonine protein kinase |
| | | | | Hypothetical Protein | | | |
| | | | | NOT Supported by cDNA | | | |
| 115 | Gma.10334.1.S1_at | 2.918 | AW234479 | Unclassified - | | | |
| | | | | Hypothetical Protein | | | |
| | | | | NOT Supported by cDNA | | | |
| 116 | Gma.6302.1.S1_at | 2.916 | BI786195 | Cell Structure | | | Beta-glucosidase, lactase |
| | | | | | | | phlorizinhydrolase, and related proteins |
| 117 | Gma.12821.1.S1_s_at | 2.903 | CD391504 | No Homology to Known | | | |
| | | | | Proteins | | | |
| 118 | GmaAffx.87812.2.S1_at | 2.898 | BI971676 | Signal Transduction | Protein Kinase | | |
| 119 | GmaAffx.14179.1.S1_at | 2.888 | BM085107 | Unclassified - | | | SETA binding protein SB1 and related |
| | | | | Hypothetical Protein | | | proteins, contain BTB/POZ domain |
| | | | | NOT Supported by cDNA | | | |
| 120 | Gma.6288.1.S1_at | 2.888 | AW348556 | Transporter | | | Aquaporin (major intrinsic protein family) |
| 121 | GmaAffx.68088.1.S1_at | 2.883 | BG507880 | Signal Transduction | Protein Kinase | | |
| 122 | Gma.3988.1.S1_at | 2.879 | BE658819 | Unclassified - | | | |
| | | | | Hypothetical Protein | | | |
| | | | | Supported by cDNA | | | |
| 123 | GmaAffx.17811.1.S1_at | 2.879 | BI426517 | Transporter | | | Predicted transporter (major facilitator |
| | | | | | | | superfamily) |
| 124 | Gma.13027.1.A1_at | 2.877 | CD397394 | Transporter | | | Amino acid transporters |
| 125 | Gma.320.1.S1_at | 2.877 | AF019116.1 | Disease & Defense | | | |
| 126 | GmaAffx.10491.1.S1_at | 2.847 | AW598141 | No Homology to Known | | | |
| | | | | Proteins | | | |
| 127 | Gma.3777.1.S1_x_at | 2.847 | CD399954 | Unclassified - | | | |
| | | | | Hypothetical Protein | | | |
| | | | | Supported by cDNA | | | |
| 128 | GmaAffx.50677.1.S1_s_at | 2.839 | BI971744 | No Homology to Known | | | |
| | | | | Proteins | | | |
| 129 | Gma.9913.2.S1_a_at | 2.822 | CD390580 | Transcription | Transcription | WRKY | |
| | | | | | Factor | | |
| 130 | Gma.7970.1.S1_at | 2.816 | BI699189 | No Homology to Known | | | |
| | | | | Proteins | | | |
| 131 | GmaAffx.74573.1.S1_at | 2.809 | BU763133 | No Homology to Known | | | |
| | | | | Proteins | | | |
| 132 | GmaAffx.42583.1.A1_at | 2.8 | BG154898 | Transporter | Inorganic | Fe(II) transporter | Fe2+/Zn2+ regulated transporter |
| | | | | | Solute | isolog family | |
| | | | | | Cotransporters | | |
| 133 | Gma.8459.1.S1_at | 2.791 | AF249317.1 | Signal Transduction | Protein Kinase | | Serine/threonine protein kinase |
| 134 | GmaAffx.9944.1.S1_at | 2.781 | BM567882 | Cell Structure | | | |
| 135 | Gma.4832.1.A1_at | 2.765 | AW310458 | Signal Transduction | Protein Kinase | | |
| 136 | Gma.17559.1.S1_at | 2.764 | BG508388 | No Homology to Known | | | |
| | | | | Proteins | | | |
| 137 | GmaAffx.93534.1.S1_s_at | 2.76 | CF809259 | Unclassified - Protein | | | |
| | | | | with Unknown Function | | | |
| 138 | Gma.17576.1.S1_s_at | 2.747 | BG650176 | No Homology to Known | | | |
| | | | | Proteins | | | |
| 139 | GmaAffx.66589.1.S1_at | 2.74 | BE821501 | Transcription | Transcription | Polycomb Group | |
| | | | | | Factor | | |
| 140 | GmaAffx.60595.1.S1_at | 2.738 | BQ628072 | No Homology to Known | | | |
| | | | | Proteins | | | |
| 141 | Gma.5621.1.S1_at | 2.71 | BU579234 | Unclassified - | | | |
| | | | | Hypothetical Protein | | | |
| | | | | NOT Supported by cDNA | | | |
| 142 | GmaAffx.52050.1.S1_at | 2.707 | BG237350 | Transcription | Transcription | MYB | Transcription factor, Myb superfamily |
| | | | | | Factor | | |
| 143 | GmaAffx.85409.1.S1_at | 2.681 | BF598665 | Unclassified - | | | Copper chaperone |
| | | | | Hypothetical Protein | | | |
| | | | | NOT Supported by cDNA | | | |
| 144 | GmaAffx.88037.2.S1_at | 2.679 | CA783510 | Unclassified - | | | |
| | | | | Hypothetical Protein | | | |
| | | | | Supported by cDNA | | | |
| 145 | Gma.17605.3.S1_at | 2.678 | BQ079859 | Secondary Metabolism | Flavonoid | | |
| | | | | | Biosynthesis | | |
| 146 | GmaAffx.91346.1.S1_x_at | 2.634 | CF807071 | Unclassified - | | | |
| | | | | Hypothetical Protein | | | |
| | | | | Supported by cDNA | | | |
| 147 | GmaAffx.89999.1.S1_at | 2.613 | CF805724 | Disease & Defense | | | |
| 148 | Gma.17816.1.A1_at | 2.607 | BI970976 | Disease & Defense | | | |
| 149 | Gma.68.1.S1_at | 2.6 | AF357211.1 | Transcription | Transcription | AP2/EREBP | |
| | | | | | Factor | | |
| 150 | Gma.7921.1.S1_at | 2.59 | BM177567 | Unclassified - | | | Uncharacterized conserved protein |
| | | | | Hypothetical Protein | | | |
| | | | | Supported by cDNA | | | |
| 151 | GmaAffx.86745.1.S1_s_at | 2.587 | BI424960 | Disease & Defense | | | |
| 152 | GmaAffx.56917.1.S1_at | 2.564 | AI441130 | Signal Transduction | Protein Kinase | | Serine/threonine protein kinase |
| 153 | Gma.9013.1.S1_at | 2.558 | AF243368.1 | Disease & Defense | | | Glutathione S-transferase |
| 154 | GmaAffx.28927.1.S1_at | 2.545 | BU549173 | Transcription | Transcription | MYB | Transcription factor, Myb superfamily |
| | | | | | Factor | | |
| 155 | GmaAffx.87812.1.S1_at | 2.543 | AW831390 | Signal Transduction | Protein Kinase | | |
| 156 | GmaAffx.10590.1.S1_s_at | 2.537 | BU551319 | Signal Transduction | Protein Kinase | | |
| 157 | Gma.4169.1.S1_at | 2.53 | BQ473559 | Transporter | | | Copper chaperone |
| 158 | GmaAffx.91346.1.S1_at | 2.513 | CF807071 | Unclassified - | | | |
| | | | | Hypothetical Protein | | | |
| | | | | Supported by cDNA | | | |
| 159 | GmaAffx.4905.1.S1_at | 2.496 | BQ453808 | Metabolism | | | Predicted lipase |
| 160 | Gma.11257.1.S1_at | 2.477 | AW310476 | Cell Structure | | | |
| 161 | GmaAffx.16502.1.A1_at | 2.47 | BE659118 | No Homology to Known | | | |
| | | | | Proteins | | | |
| 162 | GmaAffx.90946.1.S1_at | 2.462 | CF808349 | Unclassified - | | | |
| | | | | Hypothetical Protein | | | |
| | | | | NOT Supported by cDNA | | | |
| 163 | GmaAffx.47716.1.A1_at | 2.449 | CD397037 | Unclassified - | | | |
| | | | | Hypothetical Protein | | | |
| | | | | NOT Supported by cDNA | | | |
| 164 | GmaAffx.77809.1.A1_at | 2.447 | BI969302 | Disease & Defense | | | |
| 165 | GmaAffx.7215.1.A1_at | 2.443 | AW351149 | Unclassified - | | | |
| | | | | Hypothetical Protein | | | |
| | | | | NOT Supported by cDNA | | | |
| 166 | GmaAffx.13625.1.A1_at | 2.436 | BU547925 | No Homology to Known | | | |
| | | | | Proteins | | | |
| 167 | Gma.14471.4.S1_x_at | 2.434 | BQ081409 | Transporter | | | Aquaporin (major intrinsic protein family) |
| 168 | Gma.3136.1.S1_at | 2.429 | BG839241 | Metabolism | Cytochrome | CYP82C | Cytochrome P450 CYP2 subfamily |
| | | | | | P450 | | |
| 169 | GmaAffx.11658.1.S1_at | 2.427 | AW184817 | Signal Transduction | Unclassified | | |
| 170 | Gma.6529.2.S1_s_at | 2.426 | BM309390 | Unclassified - | | | |
| | | | | Hypothetical Protein | | | |
| | | | | NOT Supported by cDNA | | | |
| 171 | GmaAffX.45241.1.S1_at | 2.422 | BG157181 | Unclassified - | | | Glutaredoxin and related proteins |
| | | | | Hypothetical Protein | | | |
| | | | | Supported by cDNA | | | |
| 172 | GmaAffx.38733.1.A1_at | 2.419 | BE657590 | No Homology to Known | | | |
| | | | | Proteins | | | |
| 173 | Gma.4396.1.S1_at | 2.417 | BQ081294 | No Homology to Known | | | |
| | | | | Proteins | | | |
| 174 | Gma.9013.1.S1_x_at | 2.407 | AF243368.1 | Disease & Defense | | | Glutathione S-transferase |
| 175 | GmaAffx.79390.3.S1_at | 2.406 | BU082743 | Transcription | Transcription | Basic Leucine | FOG: Armadillo/beta-catenin-like repeats |
| | | | | | Factor | Zipper (bZIP) | |
| 176 | Gma.742.1.A1_at | 2.395 | CD393482 | Unclassified - Protein | | | |
| | | | | with Unknown Function | | | |
| 177 | Gma.1521.1.S1_at | 2.394 | CD394504 | Cell Structure | | | Beta tubulin |
| 178 | GmaAffx.89629.1.S1_s_at | 2.387 | CK606095 | Unclassified - Protein | | | |
| | | | | with Unknown Function | | | |
| 179 | Gma.12465.1.S1_a_at | 2.384 | CD415130 | Unclassified - | | | |
| | | | | Hypothetical Protein | | | |
| | | | | Supported by cDNA | | | |
| 180 | GmaAffx.5264.1.S1_s_at | 2.381 | BM521512 | Disease & Defense | | | |
| 181 | GmaAffx.18236.1.S1_at | 2.378 | BG790288 | No Homology to Known | | | |
| | | | | Proteins | | | |
| 182 | Gma.11116.4.S1_s_at | 2.373 | CD408430 | Transporter | | | Aquaporin (major intrinsic protein family) |
| 183 | Gma.2352.1.S1_at | 2.369 | AI443511 | Unclassified - Protein | | | FOG: Predicted E3 ubiquitin ligase |
| | | | | with Unknown Function | | | |
| 184 | GmaAffx.81783.1.S1_at | 2.359 | AW101961 | Signal Transduction | Protein Kinase | | Serine/threonine protein kinase |
| 185 | GmaAffx.24352.1.S1_at | 2.358 | BU544287 | No Homology to Known | | | |
| | | | | Proteins | | | |
| 186 | Gma.6650.2.S1_at | 2.354 | BU762479 | Unclassified - Protein | | | |
| | | | | with Unknown Function | | | |
| 187 | GmaAffx.33259.1.S1_at | 2.351 | BQ785322 | Unclassified - | | | |
| | | | | Hypothetical Protein | | | |
| | | | | Supported by cDNA | | | |
| 188 | Gma.8301.2.S1_at | 2.349 | BU544524 | Signal Transduction | Calcium | | |
| | | | | | Binding | | |
| 189 | GmaAffx.56804.1.A1_at | 2.318 | CD415337 | Unclassified - | | | Uncharacterized conserved protein, |
| | | | | Hypothetical Protein | | | contains TraB domain |
| | | | | NOT Supported by cDNA | | | |
| 190 | Gma.6650.1.S1_at | 2.315 | BQ629283 | Unclassified - Protein | | | |
| | | | | with Unknown Function | | | |
| 191 | GmaAffx.86059.1.S1_at | 2.302 | BF598759 | No Homology to Known | | | |
| | | | | Proteins | | | |
| 192 | GmaAffx.74289.1.S1_at | 2.295 | CA852521 | Transcription | Transcription | MYB | Transcription factor, Myb superfamily |
| | | | | | Factor | | |
| 193 | Gma.3149.1.S1_a_at | 2.287 | BU544575 | No Homology to Known | | | |
| | | | | Proteins | | | |
| 194 | Gma.9925.1.S1_at | 2.284 | AW307455 | No Homology to Known | | | |
| | | | | Proteins | | | |
| 195 | GmaAffx.87526.1.S1_at | 2.283 | BM954581 | Unclassified - | | | |
| | | | | Hypothetical Protein | | | |
| | | | | Supported by cDNA | | | |
| 196 | GmaAffx.85264.1.S1_at | 2.276 | BE058913 | Metabolism | | | |
| 197 | Gma.3189.2.S1_at | 2.261 | BG511492 | Signal Transduction | MAPKK | | Mitogen-activated protein kinase kinase |
| | | | | | | | (MAP2K) |
| 198 | Gma.14966.1.A1_at | 2.259 | CD416359 | Intracellular Traffic | | | Aquaporin (major intrinsic protein family) |
| 199 | Gma.8077.1.S1_at | 2.259 | BU547877 | Cell Structure | | | |
| 200 | Gma.43.1.A1_at | 2.256 | L12258.1 | Transporter | | | Aquaporin (major intrinsic protein family) |
| 201 | GmaAffx.30344.1.S1_at | 2.255 | AW349341 | Metabolism | | | Lipid phosphate phosphatase and related |
| | | | | | | | enzymes of the PAP2 family |
| 202 | Gma.666.1.S1_at | 2.247 | AW310734 | Unclassified - Protein | Hormone | | Voltage-gated shaker-like K+ channel, |
| | | | | with Unknown Function | Induced | | subunit beta/KCNAB |
| 203 | GmaAffx.70634.1.S1_at | 2.247 | BQ079384 | Protein Destination & | | | FOG: Predicted E3 ubiquitin ligase |
| | | | | Storage | | | |
| 204 | Gma.17347.1.S1_s_at | 2.238 | AW597676 | Transcription | Transcription | NAC Domain | |
| | | | | | Factor | | |
| 205 | Gma.17805.1.A1_s_at | 2.238 | BI968729 | Disease & Defense | | | |
| 206 | Gma.9606.1.S1_at | 2.234 | AB086392.1 | Transporter | | | Sucrose transporter and related proteins |
| 207 | Gma.18088.1.S1_at | 2.232 | BI970254 | Unclassified - Protein | | | |
| | | | | with Unknown Function | | | |
| 208 | GmaAffx.91339.1.S1_at | 2.225 | CF807064 | Cell Structure | | | |
| 209 | Gma.5139.1.S1_at | 2.218 | AF243376.1 | Secondary Metabolism | | | Glutathione S-transferase |
| 210 | Gma.14846.1.A1_at | 2.218 | AW310268 | No Homology to Known | | | |
| | | | | Proteins | | | |
| 211 | Gma.5330.2.S1_at | 2.217 | BM092298 | Unclassified - | | | |
| | | | | Hypothetical Protein | | | |
| | | | | NOT Supported by cDNA | | | |
| 212 | GmaAffx.3697.1.S1_at | 2.212 | BE820647 | Disease & Defense | | | |
| 213 | GmaAffx.83638.1.S1_at | 2.203 | BU549896 | Unclassified - | | | |
| | | | | Hypothetical Protein | | | |
| | | | | NOT Supported by cDNA | | | |
| 214 | GmaAffx.90252.1.S1_s_at | 2.201 | CF805977 | Unclassified - | | | |
| | | | | Hypothetical Protein | | | |
| | | | | Supported by cDNA | | | |
| 215 | GmaAffx.56272.1.S1_at | 2.2 | BU761188 | No Homology to Known | | | |
| | | | | Proteins | | | |
| 216 | Gma.3810.1.S1_s_at | 2.19 | BI972063 | Disease & Defense | | | |
| 217 | Gma.4368.1.S1_at | 2.185 | BQ080689 | Unclassified - Protein | | | |
| | | | | with Unknown Function | | | |
| 218 | GmaAffx.58555.1.S1_at | 2.184 | BQ298550 | Metabolism | Cytochrome | CYP82C | Cytochrome P450 CYP2 subfamily |
| | | | | | P450 | | |
| 219 | GmaAffx.73185.1.S1_at | 2.183 | BI701903 | No Homology to Known | | | |
| | | | | Proteins | | | |
| 220 | Gma.11076.1.S1_at | 2.178 | BE658385 | Unclassified - | | | |
| | | | | Hypothetical Protein | | | |
| | | | | Supported by cDNA | | | |
| 221 | GmaAffx.62710.1.S1_at | 2.167 | BE473384 | Unclassified - | | | |
| | | | | Hypothetical Protein | | | |
| | | | | NOT Supported by cDNA | | | |
| 222 | Gma.5950.1.S1_s_at | 2.166 | BQ610563 | Disease & Defense | | | |
| 223 | GmaAffx.82105.1.S1_at | 2.164 | AI938690 | Transporter | | | Sucrose transporter and related proteins |
| 224 | Gma.15523.1.S1_s_at | 2.161 | BE475550 | Cell Structure | | | |
| 225 | GmaAffx.50656.1.A1_at | 2.159 | BI970755 | No Homology to Known | | | |
| | | | | Proteins | | | |
| 226 | GmaAffx.31548.1.S1_at | 2.158 | BG044632 | Transcription | Transcription | Unclassified | Transcription factor, Myb superfamily |
| | | | | | Factor | | |
| 227 | GmaAffx.43291.1.S1_at | 2.148 | AI442332 | Metabolism | | | Lipid phosphate phosphatase and related |
| | | | | | | | enzymes of the PAP2 family |
| 228 | Gma.14802.1.S1_at | 2.144 | AW201116 | Unclassified - Protein | | | FOG: Ankyrin repeat |
| | | | | with Unknown Function | | | |
| 229 | GmaAffx.77651.1.S1_at | 2.138 | BF010272 | Unclassified - Protein | Hormone | | Zinc-binding oxidoreductase |
| | | | | with Unknown Function | Induced | | |
| 230 | GmaAffx.87547.2.S1_at | 2.133 | BQ080473 | Cell Structure | | | Beta-glucosidase, lactase |
| | | | | | | | phlorizinhydrolase, and related proteins |
| 231 | Gma.17305.1.S1_at | 2.132 | AW351319 | Unclassified - Protein | | | |
| | | | | with Unknown Function | | | |
| 232 | GmaAffx.93073.1.S1_s_at | 2.121 | CF808798 | Cell Structure | | | |
| 233 | GmaAffx.31180.1.A1_at | 2.118 | BE820254 | No Homology to Known | | | |
| | | | | Proteins | | | |
| 234 | GmaAffx.30428.1.S1_at | 2.113 | BE824410 | Protein Destination & | | | Molecular chaperones HSP70/HSC70, |
| | | | | Storage | | | HSP70 superfamily |
| 235 | GmaAffx.671.1.S1_at | 2.112 | CA785167 | Cell Structure | | | |
| 236 | GmaAffx.91185.1.S1_at | 2.103 | CF806910 | Cell Structure | | | Pectin acetylesterase and similar proteins |
| 237 | GmaAffx.79888.1.S1_at | 2.097 | AI988123 | Signal Transduction | Protein Kinase | | |
| 238 | GmaAffx.43620.1.S1_at | 2.089 | AW184956 | No Homology to Known | | | |
| | | | | Proteins | | | |
| 239 | Gma.5610.1.S1_at | 2.089 | AW348190 | Unclassified - | | | |
| | | | | Hypothetical Protein | | | |
| | | | | Supported by cDNA | | | |
| 240 | Gma.17586.1.S1_at | 2.079 | BU546955 | Unclassified - | | | Reticulon |
| | | | | Hypothetical Protein | | | |
| | | | | Supported by cDNA | | | |
| 241 | GmaAffx.3363.1.S1_at | 2.074 | BE347006 | Unclassified - | | | |
| | | | | Hypothetical Protein | | | |
| | | | | NOT Supported by cDNA | | | |
| 242 | Gma.12465.2.S1_at | 2.073 | BE611098 | Unclassified - | | | |
| | | | | Hypothetical Protein | | | |
| | | | | Supported by cDNA | | | |
| 243 | GmaAffx.57341.1.S1_at | 2.067 | AW277978 | Transporter | | | Amino acid transporters |
| 244 | GmaAffx.77404.1.S1_at | 2.067 | CD393010 | Intracellular Traffic | | | GTPase Rab11/YPT3, small G protein |
| | | | | | | | superfamily |
| 245 | Gma.869.1.S1_at | 2.061 | BE822531 | Protein Destination & | | | Serine carboxypeptidases (lysosomal |
| | | | | Storage | | | cathepsin A) |
| 246 | Gma.3576.2.S1_a_at | 2.061 | BI316058 | Unclassified - Protein | | | Diadenosine polyphosphate hydrolase and |
| | | | | with Unknown Function | | | related proteins of the histidine triad (HIT) |
| | | | | | | | family |
| 247 | Gma.612.1.A1_at | 2.06 | AI442942 | Transcription | Transcription | AP2/EREBP | |
| | | | | | Factor | | |
| 248 | Gma.3555.1.S1_at | 2.059 | BU764259 | Unclassified - | | | |
| | | | | Hypothetical Protein | | | |
| | | | | NOT Supported by cDNA | | | |
| 249 | GmaAffx.8013.1.S1_at | 2.053 | BE609266 | Unclassified - | | | |
| | | | | Hypothetical Protein | | | |
| | | | | NOT Supported by cDNA | | | |
| 250 | Gma.10441.1.S1_s_at | 2.031 | BE803701 | Unclassified - Protein | | | |
| | | | | with Unknown Function | | | |
| 251 | GmaAffx.47891.1.S1_s_at | 2.03 | BQ785880 | Cell Structure | | | |
| 252 | GmaAffx.79401.1.S1_at | 2.027 | CA936959 | Transcription | Transcription | bZIP | |
| | | | | | Factor | | |
| 253 | GmaAffx.70056.1.S1_at | 2.018 | BU765705 | No Homology to Known | | | |
| | | | | Proteins | | | |
| 254 | GmaAffx.18518.1.S1_at | 2.016 | BI700329 | Cell Structure | | | |
| 255 | GmaAffx.46974.1.S1_at | 2.014 | BU081847 | Signal Transduction | Protein Kinase | | |
| 256 | Gma.13613.1.A1_at | 2.012 | CD401987 | No Homology to Known | | | |
| | | | | Proteins | | | |
| 257 | GmaAffx.23953.1.S1_s_at | 2.005 | BE658262 | Transporter | | | |
| 258 | GmaAffx.45853.1.S1_at | 2.005 | BQ081229 | Unclassified - Protein | | | |
| | | | | with Unknown Function | | | |
| 259 | Gma.13973.1.A1_at | 2.004 | CD412168 | No Homology to Known | | | |
| | | | | Proteins | | | |
| 260 | GmaAffx.72630.1.S1_at | 2.003 | BG239729 | Unclassified - | | | FOG: TPR repeat |
| | | | | Hypothetical Protein | | | |
| | | | | NOT Supported by cDNA | | | |
| 260 | GmaAffx.72630.1.S1_at | 2.003 | BG239729 | Unclassified - | | | Thioredoxin |
| | | | | Hypothetical Protein | | | |
| | | | | NOT Supported by cDNA | | | |
| 261 | Gma.6803.2.S1_s_at | 0.5 | BU082740 | Disease & Defense | | | |
| 262 | GmaAffx.45003.1.S1_at | 0.499 | BI968048 | Unclassified - | | | Uncharacterized conserved protein |
| | | | | Hypothetical Protein | | | |
| | | | | Supported by cDNA | | | |
| 263 | Gma.2952.1.S1_at | 0.499 | BM270250 | No Homology to Known | | | |
| | | | | Proteins | | | |
| 264 | GmaAffx.28397.1.S1_at | 0.499 | BM568229 | Metabolism | | | |
| 265 | GmaAffx.71944.1.S1_at | 0.498 | AW351155 | Unclassified - | | | |
| | | | | Hypothetical Protein | | | |
| | | | | Supported by cDNA | | | |
| 266 | Gma.6375.1.S1_at | 0.498 | BQ610190 | Unclassified - | | | AAA+-type ATPase |
| | | | | Hypothetical Protein | | | |
| | | | | NOT Supported by cDNA | | | |
| 267 | Gma.11028.2.S1_s_at | 0.498 | CA782711 | Metabolism | | | Glyoxylate/hydroxypyruvate reductase (D- |
| | | | | | | | isomer-specific 2-hydroxy acid |
| | | | | | | | dehydrogenase superfamily) |
| 268 | Gma.1528.1.S1_at | 0.498 | CD403623 | Transcription | Transcription | Basic Helix- | |
| | | | | | Factor | Loop-Helix | |
| | | | | | | (bHLH) | |
| 269 | GmaAffx.85469.1.S1_at | 0.497 | BI786397 | Unclassified - | | | |
| | | | | Hypothetical Protein | | | |
| | | | | NOT Supported by cDNA | | | |
| 270 | GmaAffx.90069.1.S1_at | 0.494 | CF805794 | Unclassified - | | | Leucine rich repeat proteins, some proteins |
| | | | | Hypothetical Protein | | | contain F-box |
| | | | | Supported by cDNA | | | |
| 271 | GmaAffx.79516.1.A1_at | 0.494 | CD400596 | Transcription | Transcription | MYB | |
| | | | | | Factor | | |
| 272 | GmaAffx.37930.1.A1_at | 0.494 | BU547709 | Cell Structure | | | |
| 273 | Gma.3744.1.S1_at | 0.494 | CD417438 | Unclassified - Protein | | | |
| | | | | With Unknown Function | | | |
| 274 | Gma.1642.3.S1_s_at | 0.493 | BM891895 | Unclassified - | | | |
| | | | | Hypothetical Protein | | | |
| | | | | Supported by cDNA | | | |
| 275 | Gma.7371.2.S1_a_at | 0.493 | BQ741076 | Cell Structure | | | Dynein light chain type 1 |
| 276 | Gma.16521.1.S1_at | 0.493 | BE609300 | Unclassified - | | | Uncharacterized conserved protein |
| | | | | Hypothetical Protein | | | |
| | | | | Supported by cDNA | | | |
| 277 | Gma.13335.1.S1_at | 0.492 | CD417489 | Unclassified - | | | |
| | | | | Hypothetical Protein | | | |
| | | | | Supported by cDNA | | | |
| 278 | GmaAffx.7302.2.A1_s_at | 0.488 | BE820704 | No Homology to Known | | | |
| | | | | Proteins | | | |
| 279 | Gma.13039.1.A1_at | 0.488 | CD399750 | Unclassified - | | | |
| | | | | Hypothetical Protein | | | |
| | | | | NOT Supported by cDNA | | | |
| 280 | GmaAffx.35729.1.S1_s_at | 0.488 | BE806047 | Unclassified - | | | Predicted E3 ubiquitin ligase |
| | | | | Hypothetical Protein | | | |
| | | | | Supported by cDNA | | | |
| 281 | GmaAffx.81502.2.S1_at | 0.486 | BG041291 | Unclassified - | | | Uncharacterized conserved protein |
| | | | | Hypothetical Protein | | | |
| | | | | NOT Supported by cDNA | | | |
| 282 | GmaAffx.63115.1.A1_at | 0.485 | BE823736 | Unclassified - | | | |
| | | | | Hypothetical Protein | | | |
| | | | | NOT Supported by cDNA | | | |
| 283 | Gma.4315.1.S1_at | 0.485 | BM886786 | Unclassified - Protein | | | |
| | | | | with Unknown Function | | | |
| 284 | Gma.3591.1.S1_a_at | 0.485 | BE440266 | Secondary Metabolism | Ethylene | | Iron/ascorbate family oxidoreductases |
| | | | | | Biosynthesis | | |
| 285 | GmaAffx.33527.1.S1_at | 0.485 | BU761195 | No Homology to Known | | | |
| | | | | Proteins | | | |
| 286 | GmaAffx.53069.2.S1_at | 0.483 | AW309012 | No Homology to Known | | | |
| | | | | Proteins | | | |
| 287 | GmaAffx.91731.1.S1_s_at | 0.483 | CF807456 | Secondary Metabolism | Ethylene | | Iron/ascorbate family oxidoreductases |
| | | | | | Biosynthesis | | |
| 288 | GmaAffx.90242.1.S1_s_at | 0.483 | CF805967 | No Homology to Known | | | |
| | | | | Proteins | | | |
| 289 | Gma.7422.1.S1_at | 0.482 | BQ612005 | Transporter | | | |
| 290 | Gma.3744.2.S1_a_at | 0.481 | BI892693 | Unclassified - Protein | | | |
| | | | | With Unknown Function | | | |
| 291 | Gma.13418.3.S1_at | 0.481 | CA938391 | Transporter | | | |
| 292 | GmaAffx.73857.1.S1_at | 0.48 | BM885538 | Unclassified - | | | |
| | | | | Hypothetical Protein | | | |
| | | | | Supported by cDNA | | | |
| 293 | GmaAffx.50635.2.S1_at | 0.48 | AW733509 | Metabolism | Cytochrome | CYP707A | Cytochrome P450 CYP4/CYP19/CYP26 |
| | | | | | P450 | | subfamilies |
| 294 | GmaAffx.83103.2.S1_at | 0.48 | BE806704 | Unclassified - | | | |
| | | | | Hypothetical Protein | | | |
| | | | | NOT Supported by cDNA | | | |
| 295 | GmaAffx.86667.1.S1_at | 0.48 | CA938472 | Unclassified - | | | |
| | | | | Hypothetical Protein | | | |
| | | | | Supported by cDNA | | | |
| 296 | GmaAffx.93157.1.S1_s_at | 0.479 | CF808882 | Metabolism | | | Aldo/keto reductase family proteins |
| 297 | GmaAffx.37803.2.S1_at | 0.478 | BE347017 | No Homology to Known | | | |
| | | | | Proteins | | | |
| 298 | GmaAffx.71032.1.S1_at | 0.477 | AW278168 | Unclassified - | | | |
| | | | | Hypothetical Protein | | | |
| | | | | NOT Supported by cDNA | | | |
| 299 | Gma.17007.1.S1_at | 0.477 | BE820533 | No Homology to Known | | | |
| | | | | Proteins | | | |
| 300 | GmaAffx.76295.1.S1_at | 0.476 | BE347326 | Unclassified - Protein | Hormone | | |
| | | | | with Unknown Function | Induced | | |
| 301 | GmaAffx.74390.1.A1_at | 0.476 | BU544169 | Protein Destination & | | | Molecular chaperone (DnaJ superfamily) |
| | | | | Storage | | | |
| 302 | Gma.16842.1.A1_at | 0.475 | CD392652 | No Homology to Known | | | |
| | | | | Proteins | | | |
| 303 | Gma.4874.1.A1_at | 0.475 | AI938293 | No Homology to Known | | | |
| | | | | Proteins | | | |
| 304 | GmaAffx.25612.1.S1_at | 0.475 | AW317577 | No Homology to Known | | | |
| | | | | Proteins | | | |
| 305 | GmaAffx.30593.1.S1_at | 0.474 | BE803365 | Unclassified - | | | |
| | | | | Hypothetical Protein | | | |
| | | | | Supported by cDNA | | | |
| 306 | GmaAffx.33677.1.S1_at | 0.474 | CA782582 | No Homology to Known | | | |
| | | | | Proteins | | | |
| 307 | GmaAffx.76699.1.S1_at | 0.473 | AW760679 | Transcription | Transcription | Basic Helix- | |
| | | | | | Factor | Loop-Helix | |
| | | | | | | (bHLH) | |
| 308 | Gma.4340.2.S1_a_at | 0.473 | BE611760 | Secondary Metabolism | | | Hydroxyindole-O-methyltransferase and |
| | | | | | | | related SAM-dependent methyltransferases |
| 309 | GmaAffx.38961.1.S1_at | 0.473 | BU081740 | Transcription | Transcription | MYB | Transcription factor, Myb superfamily |
| | | | | | Factor | | |
| 310 | GmaAffx.78358.2.S1_at | 0.472 | BU762686 | Metabolism | | | |
| 311 | Gma.2565.1.S1_at | 0.471 | BG652533 | Unclassified - | | | |
| | | | | Hypothetical Protein | | | |
| | | | | Supported by cDNA | | | |
| 312 | GmaAffx.53033.1.S1_at | 0.471 | BG046837 | Transcription | Transcription | AP2/EREBP | |
| | | | | | Factor | | |
| 313 | GmaAffx.79730.1.S1_at | 0.471 | BF325482 | Transcription | Transcription | Basic Helix- | |
| | | | | | Factor | Loop-Helix | |
| | | | | | | (bHLH) | |
| 314 | Gma.15625.1.S1_a_at | 0.467 | U77678.1 | Metabolism | Amino Acid | | Asparagine synthase (glutamine- |
| | | | | | Biosynthesis/ | | hydrolyzing) |
| | | | | | Metabolism | | |
| 315 | GmaAffx.59808.1.S1_at | 0.467 | BM886228 | Transporter | | | Predicted transporter (major facilitator |
| | | | | | | | superfamily) |
| 316 | Gma.16196.1.S1_at | 0.467 | AW350888 | Transcription | Transcription | Unclassified | |
| | | | | | Factor | | |
| 317 | Gma.12641.1.A1_at | 0.467 | CD393495 | No Homology to Known | | | |
| | | | | Proteins | | | |
| 318 | GmaAffx.57469.1.S1_at | 0.467 | AW351054 | Unclassified - | | | |
| | | | | Hypothetical Protein | | | |
| | | | | Supported by cDNA | | | |
| 319 | GmaAffx.23475.2.S1_s_at | 0.467 | AW620563 | Unclassified - | | | |
| | | | | Hypothetical Protein | | | |
| | | | | Supported by cDNA | | | |
| 320 | Gma.7256.1.S1_at | 0.466 | L01447.1 | Transcription | Transcription | Basic Leucine | |
| | | | | | Factor | Zipper (bZIP) | |
| 321 | Gma.13103.1.S1_at | 0.466 | CD392133 | Unclassified - | | | |
| | | | | Hypothetical Protein | | | |
| | | | | NOT Supported by cDNA | | | |
| 322 | Gma.4643.1.S1_at | 0.466 | BU547906 | Transcription | Transcription | NAC Domain | |
| | | | | | Factor | | |
| 323 | Gma.11665.2.A1_at | 0.465 | BU545959 | No Homology to Known | | | |
| | | | | Proteins | | | |
| 324 | GmaAffx.74256.1.S1_at | 0.463 | BE057293 | No Homology to Known | | | |
| | | | | Proteins | | | |
| 325 | Gma.7040.1.A1_at | 0.462 | BE346424 | Transcription | Transcription | AP2/EREBP | |
| | | | | | Factor | | |
| 326 | Gma.13146.1.S1_at | 0.461 | BM731716 | Unclassified - | | | |
| | | | | Hypothetical Protein | | | |
| | | | | Supported by cDNA | | | |
| 327 | GmaAffx.48042.1.S1_at | 0.461 | AI960210 | Transporter | | | Aquaporin (major intrinsic protein family) |
| 328 | GmaAffx.27488.1.S1_at | 0.459 | AW311080 | Signal Transduction | MAPKKK | Raf | Tyrosine kinase specific for activated (GTP- |
| | | | | | | | bound) p21cdc42Hs |
| 329 | GmaAffx.71386.1.S1_at | 0.458 | AW704747 | Protein Destination & | | | Molecular chaperone (DnaJ superfamily) |
| | | | | Storage | | | |
| 330 | GmaAffx.6017.1.A1_at | 0.458 | BU550467 | Unclassified - | | | |
| | | | | Hypothetical Protein | | | |
| | | | | NOT Supported by cDNA | | | |
| 331 | GmaAffx.68318.1.S1_at | 0.457 | BU545357 | Transcription | Transcription | AUX/1AA | |
| | | | | | Factor | | |
| 332 | GmaAffx.87295.1.S1_at | 0.455 | BM519660 | No Homology to Known | | | |
| | | | | Proteins | | | |
| 333 | Gma.2718.1.S1_at | 0.455 | CD406302 | Transporter | | | Multidrug resistance-associated |
| | | | | | | | protein/mitoxantrone resistance protein, |
| | | | | | | | ABC superfamily |
| 334 | GmaAffx.53256.1.S1_at | 0.454 | BU545086 | Unclassified - | | | |
| | | | | Hypothetical Protein | | | |
| | | | | Supported by cDNA | | | |
| 335 | GmaAffx.53069.1.S1_at | 0.454 | AW733700 | Unclassified - | | | |
| | | | | Hypothetical Protein | | | |
| | | | | NOT Supported by cDNA | | | |
| 336 | GmaAffx.93308.1.S1_at | 0.454 | CF809033 | Protein Destination & | | | Glutathione S-transferase |
| | | | | Storage | | | |
| 337 | GmaAffx.53099.1.S1_at | 0.454 | BG043340 | Unclassified - | | | Alpha-L-fucosidase |
| | | | | Hypothetical Protein | | | |
| | | | | NOT Supported by cDNA | | | |
| 338 | GmaAffx.13730.1.S1_at | 0.453 | BI316755 | Transcription | Transcription | Basic Helix- | |
| | | | | | Factor | Loop-Helix | |
| | | | | | | (bHLH) | |
| 339 | GmaAffx.70435.2.S1_at | 0.453 | AW705853 | Transcription | Transcription | Basic Helix- | |
| | | | | | Factor | Loop-Helix | |
| | | | | | | (bHLH) | |
| 340 | Gma.6659.1.S1_at | 0.452 | BQ629137 | Unclassified - Protein | | | Protein involved in membrane traffic |
| | | | | with Unknown Function | | | (YOP1/TB2/DP1/HVA22 family) |
| 341 | GmaAffx.55657.1.S1_at | 0.451 | BM885600 | No Homology to Known | | | |
| | | | | Proteins | | | |
| 342 | GmaAffx.76696.1.A1_at | 0.451 | BE824386 | No Homology to Known | | | |
| | | | | Proteins | | | |
| 343 | GmaAffx.90899.1.S1_at | 0.447 | CF806624 | Unclassified - | | | |
| | | | | Hypothetical Protein | | | |
| | | | | NOT Supported by cDNA | | | |
| 344 | GmaAffx.5232.1.S1_at | 0.447 | BM309521 | Unclassified - Protein | | | |
| | | | | with Unknown Function | | | |
| 345 | Gma.17686.1.S1_at | 0.447 | BF597378 | Metabolism | Cytochrome | CYP76C | Cytochrome P450 CYP2 subfamily |
| | | | | | P450 | | |
| 346 | Gma.1195.1.S1_s_at | 0.447 | AI442801 | Metabolism | | | Predicted flavoprotein |
| 347 | GmaAffx.73303.1.S1_at | 0.446 | BE190156 | Unclassified - | | | Predicted membrane protein |
| | | | | Hypothetical Protein | | | |
| | | | | Supported by cDNA | | | |
| 348 | Gma.6804.1.S1_at | 0.446 | BE657581 | Protein Destination & | | | Aspartyl protease |
| | | | | Storage | | | |
| 349 | GmaAffx.74160.1.S1_at | 0.446 | BQ740845 | Protein Destination & | | | |
| | | | | Storage | | | |
| 350 | Gma.1951.2.S1_at | 0.445 | CA783485 | Protein Destination & | | | Serine carboxypeptidases |
| | | | | Storage | | | |
| 351 | Gma.10591.2.S1_at | 0.444 | CD414199 | Metabolism | | | Glycine cleavage system H protein (lipoate- |
| | | | | | | | binding) |
| 352 | Gma.5760.2.S1_at | 0.444 | BQ298381 | Disease & Defense | | | |
| 353 | GmaAffx.25912.1.S1_at | 0.443 | BU545028 | Unclassified - Protein | | | |
| | | | | with Unknown Function | | | |
| 354 | GmaAffx.74693.1.S1_at | 0.442 | CD406171 | Metabolism | | | |
| 355 | GmaAffx.69307.1.S1_at | 0.442 | BQ473855 | No Homology to Known | | | |
| | | | | Proteins | | | |
| 356 | Gma.2066.1.S1_a_at | 0.44 | BG363704 | No Homology to Known | | | |
| | | | | Proteins | | | |
| 357 | GmaAffx.26683.1.A1_at | 0.439 | BE820379 | No Homology to Known | | | |
| | | | | Proteins | | | |
| 358 | Gma.11194.2.S1_at | 0.438 | BM886635 | Energy | | | Thioredoxin |
| 359 | Gma.10100.1.S1_at | 0.438 | BE023275 | No Homology to Known | | | |
| | | | | Proteins | | | |
| 360 | Gma.3880.1.S1_at | 0.437 | BE659305 | Unclassified - | | | |
| | | | | Hypothetical Protein | | | |
| | | | | NOT Supported by cDNA | | | |
| 361 | Gma.13241.1.S1_at | 0.434 | CD395739 | Unclassified - | | | |
| | | | | Hypothetical Protein | | | |
| | | | | NOT Supported by cDNA | | | |
| 362 | GmaAffx.13016.1.S1_at | 0.434 | BF069324 | No Homology to Known | | | |
| | | | | Proteins | | | |
| 363 | Gma.2827.1.S1_at | 0.433 | BG237560 | Unclassified - | | | FOG: PPR repeat |
| | | | | Hypothetical Protein | | | |
| | | | | Supported by cDNA | | | |
| 364 | GmaAffx.56319.1.S1_at | 0.433 | BE802287 | Metabolism | | | Uncharacterized conserved protein |
| 365 | GmaAffx.26667.1.S1_at | 0.432 | BE806932 | No Homology to Known | | | |
| | | | | Proteins | | | |
| 366 | Gma.6129.2.S1_s_at | 0.432 | AW186006 | Protein Synthesis | | | Translation initiation factor 5A (eIF-5A) |
| 367 | GmaAffx.8304.1.A1_at | 0.431 | BE822695 | Unclassified - | | | Possible oxidoreductase |
| | | | | Hypothetical Protein | | | |
| | | | | Supported by cDNA | | | |
| 368 | Gma.4305.1.S1_at | 0.431 | AW350687 | Disease & Defense | | | |
| 369 | GmaAffx.87900.1.S1_at | 0.429 | BI892943 | Protein Destination & | | | FKBP-type peptidyl-prolyl cis-trans |
| | | | | Storage | | | isomerase |
| 370 | GmaAffx.90113.1.S1_at | 0.428 | CF806004 | Protein Destination & | Storage | | Uncharacterized conserved protein |
| | | | | Storage | Protein | | |
| 371 | GmaAffx.59210.1.S1_at | 0.428 | BI967182 | Secondary Metabolism | Ethylene | | Iron/ascorbate family oxidoreductases |
| | | | | | Biosynthesis | | |
| 372 | Gma.7227.1.S1_at | 0.428 | BU551112 | Protein Destination & | | | Ubiquitin-protein ligase |
| | | | | Storage | | | |
| 373 | Gma.10940.1.S1_at | 0.426 | BU550016 | Unclassified - | | | Metalloendopeptidase family - |
| | | | | Hypothetical Protein | | | saccharolysin & thimet oligopeptidase |
| | | | | Supported by cDNA | | | |
| 374 | Gma.2887.2.S1_x_at | 0.426 | BI701520 | Secondary Metabolism | | | Phenylalanine and histidine ammonia-lyase |
| 375 | Gma.17191.1.S1_at | 0.426 | BG237724 | Metabolism | | | UDP-glucuronosyl and UDP-glucosyl |
| | | | | | | | transferase |
| 376 | GmaAffx.84144.1.A1_s_at | 0.426 | CD414061 | No Homology to Known | | | |
| | | | | Proteins | | | |
| 377 | GmaAffx.58073.1.S1_at | 0.423 | CA852105 | Unclassified - | | | |
| | | | | Hypothetical Protein | | | |
| | | | | Supported by cDNA | | | |
| 378 | Gma.7549.1.S1_at | 0.423 | BG508637 | Unclassified - Protein | | | |
| | | | | with Unknown Function | | | |
| 379 | GmaAffx.69792.1.S1_s_at | 0.423 | BU550596 | Transcription | Transcription | AP2/EREBP | |
| | | | | | Factor | | |
| 380 | Gma.3165.1.S1_at | 0.422 | CD406755 | Transcription | Transcription | Homeodomain | Transcription factor HEX, contains HOX |
| | | | | | Factor | | and HALZ domains |
| 381 | Gma.10393.1.S1_at | 0.422 | BE804608 | Unclassified - | | | Endoplasmic reticulum protein EP58, |
| | | | | Hypothetical Protein | | | contains filamin rod domain and KDEL |
| | | | | NOT Supported by cDNA | | | motif |
| 382 | GmaAffx.948.1.S1_at | 0.421 | BG155389 | Protein Destination & | | | Ubiquitin fusion-degradation protein |
| | | | | Storage | | | |
| 383 | GmaAffx.21041.1.A1_at | 0.421 | AW350819 | No Homology to Known | | | |
| | | | | Proteins | | | |
| 384 | GmaAffx.1301.68.S1_s_at | 0.421 | BI974374 | Intracellular Traffic | | | Annexin |
| 385 | GmaAffx.15471.1.S1_at | 0.42 | BQ611747 | Transcription | Transcription | MYB | Transcription factor, Myb superfamily |
| | | | | | Factor | | |
| 386 | GmaAffx.6017.2.S1_at | 0.419 | BI469662 | Unclassified - | | | |
| | | | | Hypothetical Protein | | | |
| | | | | NOT Supported by cDNA | | | |
| 387 | Gma.10536.1.S1_at | 0.419 | BG508300 | No Homology to Known | | | |
| | | | | Proteins | | | |
| 388 | PsAffx.psHB026xH01f_s_at | 0.419 | | | | | |
| 389 | Gma.16947.1.S1_at | 0.419 | BG155375 | Unclassified - | | | |
| | | | | Hypothetical Protein | | | |
| | | | | Supported by cDNA | | | |
| 390 | Gma.1490.1.S1_at | 0.418 | AB000097.1 | Cell Structure | | | Chitinase |
| 391 | Gma.8501.1.S1_at | 0.418 | AF184278.1 | Transcription | Transcription | Homeodomain | Transcription factor HEX, contains HOX |
| | | | | | Factor | | and HALZ domains |
| 392 | GmaAffx.12014.1.A1_at | 0.418 | CA802431 | Unclassified - | | | NADH pyrophosphatase 1 of the Nudix |
| | | | | Hypothetical Protein | | | family of hydrolases |
| | | | | Supported by cDNA | | | |
| 393 | GmaAffx.83226.1.S1_at | 0.417 | CA852250 | Transcription | Transcription | Unclassified | |
| | | | | | Factor | | |
| 394 | GmaAffx.52391.1.S1_at | 0.416 | AW349563 | Unclassified - | | | |
| | | | | Hypothetical Protein | | | |
| | | | | NOT Supported by cDNA | | | |
| 395 | GmaAffx.34657.1.A1_s_at | 0.415 | AW351012 | No Homology to Known | | | |
| | | | | Proteins | | | |
| 396 | Gma.11731.1.A1_at | 0.414 | CD392320 | Unclassified - Protein | | | |
| | | | | with Unknown Function | | | |
| 397 | GmaAffx.74500.1.A1_at | 0.413 | BU551117 | No Homology to Known | | | |
| | | | | Proteins | | | |
| 398 | GmaAffx.1301.59.S1_at | 0.412 | BI974591 | Intracellular Traffic | | | Annexin |
| 399 | Gma.3578.1.S1_s_at | 0.412 | AW156522 | Disease & Defense | | | Acyl-CoA synthetase |
| 400 | Gma.5513.1.S1_at | 0.412 | AW310453 | Metabolism | | | Haloacid dehalogenase-like hydrolase |
| 401 | Gma.5294.1.S1_s_at | 0.411 | BE658252 | Unclassified - | | | |
| | | | | Hypothetical Protein | | | |
| | | | | Supported by cDNA | | | |
| 402 | GmaAffx.84566.3.S1_s_at | 0.411 | BG045916 | Transcription | Transcription | MYB | Transcription factor, Myb superfamily |
| | | | | | Factor | | |
| 403 | GmaAffx.11999.1.S1_at | 0.411 | AW567661 | No Homology to Known | | | |
| | | | | Proteins | | | |
| 404 | GmaAffx.5255.1.S1_at | 0.41 | BG362929 | Transcription | Transcription | AP2/EREBP | |
| | | | | | Factor | | |
| 405 | GmaAffx.25068.2.A1_at | 0.409 | CD413732 | Metabolism | Amino Acid | | |
| | | | | | Biosynthesis/ | | |
| | | | | | Metabolism | | |
| 406 | Gma.595.1.S1_at | 0.407 | AB004271.1 | Metabolism | | | |
| 407 | Gma.5513.1.S1_x_at | 0.406 | AW310453 | Metabolism | | | Haloacid dehalogenase-like hydrolase |
| 408 | GmaAffx.86613.2.S1_at | 0.404 | BE024005 | Metabolism | | | Glutaredoxin and related proteins |
| 409 | Gma.4099.1.S1_at | 0.404 | BG651846 | Metabolism | | | Beta-galactosidase |
| 410 | GmaAffx.60479.1.S1_at | 0.403 | BG726023 | Transcription | Transcription | Homeodomain | Transcription factor MEIS1 and related |
| | | | | | Factor | | HOX domain proteins |
| 411 | Gma.13019.1.A1_s_at | 0.402 | CD392967 | Secondary Metabolism | | | Hydroxyindole-O-methyltransferase and |
| | | | | | | | related SAM-dependent methyltransferases |
| 412 | Gma.6895.1.S1_a_at | 0.402 | BU545788 | Metabolism | | | Alanine-glyoxylate aminotransferase AGT2 |
| 413 | GmaAffx.31838.1.S1_at | 0.402 | BU547835 | Secondary Metabolism | Flavonoid | | Flavonol reductase/cinnamoyl-CoA |
| | | | | | Biosynthesis | | reductase |
| 414 | Gma.4249.1.S1_at | 0.401 | BF324893 | Secondary Metabolism | | | Methyltransferase |
| 415 | GmaAffx.14141.1.A1_at | 0.401 | BI973009 | No Homology to Known | | | |
| | | | | Proteins | | | |
| 416 | GmaAffx.11496.1.S1_at | 0.4 | BE821691 | Transporter | Inorganic | Putative sulfate | Sulfate/bicarbonate/oxalate exchanger SAT- |
| | | | | | Solute | transporter | 1 and related transporters (SLC26 family) |
| | | | | | Cotransporters | family | |
| 417 | GmaAffx.494.1.S1_at | 0.4 | AI938330 | Cell Structure | | | Chitinase |
| 418 | Gma.3065.1.S1_s_at | 0.399 | AW349564 | Unclassified - | | | |
| | | | | Hypothetical Protein | | | |
| | | | | Supported by cDNA | | | |
| 419 | GmaAffx.52526.1.S1_at | 0.399 | BM085509 | Unclassified - | | | Uncharacterized conserved protein |
| | | | | Hypothetical Protein | | | |
| | | | | Supported by cDNA | | | |
| 420 | Gma.17402.1.A1_at | 0.398 | BE611084 | No Homology to Known | | | |
| | | | | Proteins | | | |
| 421 | GmaAffx.37316.1.S1_at | 0.398 | AW733720 | Unclassified - Protein | | | |
| | | | | with Unknown Function | | | |
| 422 | Gma.137.1.S1_at | 0.397 | AF022464.1 | Metabolism | Cytochrome | | Cytochrome P450 CYP2 subfamily |
| | | | | | P450 | | |
| 423 | Gma.17594.2.S1_at | 0.397 | BM093196 | Metabolism | | | Cystathionine beta-lyases/cystathionine |
| | | | | | | | gamma-synthases |
| 424 | Gma.10873.1.S1_s_at | 0.395 | BQ079465 | No Homology to Known | | | |
| | | | | Proteins | | | |
| 425 | GmaAffx.3369.1.S1_at | 0.395 | BU548983 | Cell Structure | | | |
| 426 | Gma.4728.1.A1_at | 0.394 | BI968944 | Secondary Metabolism | | | NADH:flavin oxidoreductase/12- |
| | | | | | | | oxophytodienoate reductase |
| 427 | GmaAffx.86212.1.S1_at | 0.394 | BG043474 | Unclassified - | | | |
| | | | | Hypothetical Protein | | | |
| | | | | NOT Supported by cDNA | | | |
| 428 | GmaAffx.62153.1.S1_at | 0.394 | AW394575 | No Homology to Known | | | |
| | | | | Proteins | | | |
| 429 | Gma.6264.1.S1_at | 0.393 | BQ612558 | Unclassified - | | | |
| | | | | Hypothetical Protein | | | |
| | | | | Supported by cDNA | | | |
| 430 | GmaAffx.37988.1.S1_at | 0.392 | BF324942 | No Homology to Known | | | |
| | | | | Proteins | | | |
| 431 | GmaAffx.72807.1.S1_at | 0.391 | AW831487 | No Homology to Known | | | |
| | | | | Proteins | | | |
| 432 | Gma.16875.1.A1_at | 0.39 | CD406723 | Metabolism | | | Aldo/keto reductase family proteins |
| 433 | GmaAffx.70659.1.S1_at | 0.39 | AI495240 | No Homology to Known | | | |
| | | | | Proteins | | | |
| 434 | GmaAffx.70005.1.S1_s_at | 0.39 | CA936335 | Secondary Metabolism | | | Iron/ascorbate family oxidoreductases |
| 435 | GmaAffx.91358.1.S1_x_at | 0.39 | CF807083 | No Homology to Known | | | |
| | | | | Proteins | | | |
| 436 | GmaAffx.32011.1.S1_at | 0.389 | BI701198 | No Homology to Known | | | |
| | | | | Proteins | | | |
| 437 | GmaAffx.80584.1.S1_at | 0.388 | BU083387 | Transcription | Transcription | Basic Helix- | |
| | | | | | Factor | Loop-Helix | |
| | | | | | | (bHLH) | |
| 438 | GmaAffx.70548.1.A1_at | 0.388 | CD411723 | No Homology to Known | | | |
| | | | | Proteins | | | |
| 439 | Gma.6033.2.S1_at | 0.387 | AI416728 | Unclassified - | | | |
| | | | | Hypothetical Protein | | | |
| | | | | Supported by cDNA | | | |
| 440 | GmaAffx.93328.1.S1_s_at | 0.387 | CF809053 | Secondary Metabolism | | | |
| 441 | Gma.16359.1.A1_at | 0.387 | BI970359 | Metabolism | | | GPI-alpha-mannosyltransferase III |
| | | | | | | | (GPI10/PIG-B) involved in |
| | | | | | | | glycosylphosphatidylinositol anchor |
| | | | | | | | biosynthesis |
| 442 | GmaAffx.71038.1.S1_at | 0.386 | BI700350 | Transcription | Transcription | Homeodomain | Transcription factor MEIS1 and related |
| | | | | | Factor | | HOX domain proteins |
| 443 | GmaAffx.68507.1.S1_at | 0.384 | BI699405 | Transcription | Transcription | Heat Shock | Heat shock transcription factor |
| | | | | | Factor | | |
| 444 | GmaAffx.87793.1.S1_at | 0.384 | BI941782 | Unclassified - | | | |
| | | | | Hypothetical Protein | | | |
| | | | | NOT Supported by cDNA | | | |
| 445 | Gma.5701.3.S1_at | 0.384 | CA852693 | No Homology to Known | | | |
| | | | | Proteins | | | |
| 446 | GmaAffx.48438.1.S1_at | 0.384 | AW350858 | Protein Destination & | | | Molecular chaperone (DnaJ superfamily) |
| | | | | Storage | | | |
| 447 | GmaAffx.15792.1.A1_at | 0.384 | AW101026 | No Homology to Known | | | |
| | | | | Proteins | | | |
| 448 | Gma.3204.1.S1_at | 0.383 | AW509002 | Metabolism | | | Dehydrogenase kinase |
| 449 | Gma.4506.1.S1_at | 0.382 | BQ740943 | Unclassified - | | | |
| | | | | Hypothetical Protein | | | |
| | | | | NOT Supported by cDNA | | | |
| 450 | GmaAffx.58125.1.A1_at | 0.381 | BE347561 | Transcription | Transcription | Basic Helix- | |
| | | | | | Factor | Loop-Helix | |
| | | | | | | (bHLH) | |
| 451 | Gma.17070.1.S1_at | 0.381 | BE822463 | Disease & Defense | | | O-methyltransferase |
| 452 | GmaAffx.74933.1.S1_at | 0.38 | CA935084 | Unclassified - Protein | | | Aspartyl protease |
| | | | | with Unknown Function | | | |
| 453 | GmaAffx.84964.1.S1_at | 0.379 | AW598267 | No Homology to Known | | | |
| | | | | Proteins | | | |
| 454 | GmaAffx.82034.1.S1_at | 0.379 | BM887093 | Transcription | Transcription | Homeodomain | Transcription factor HEX, contains HOX |
| | | | | | Factor | | and HALZ domains |
| 455 | Gma.4687.1.S1_at | 0.379 | AW309716 | Secondary Metabolism | | | |
| 456 | Gma.13187.1.S1_at | 0.379 | BG839239 | Unclassified - | | | Uncharacterized conserved protein |
| | | | | Hypothetical Protein | | | |
| | | | | Supported by cDNA | | | |
| 457 | Gma.3384.1.S1_at | 0.378 | BE659400 | Unclassified - Protein | | | Acyl-CoA reductase |
| | | | | with Unknown Function | | | |
| 458 | GmaAffx.7838.3.S1_s_at | 0.378 | CA800410 | Metabolism | | | |
| 459 | GmaAffx.40114.1.A1_at | 0.378 | BE661587 | Unclassified - | | | |
| | | | | Hypothetical Protein | | | |
| | | | | Supported by cDNA | | | |
| 460 | GmaAffx.85657.1.S1_s_at | 0.378 | AW185911 | Unclassified - Protein | | | FOG: Kelch repeat |
| | | | | with Unknown Function | | | |
| 461 | Gma.17798.2.S1_at | 0.376 | CD412748 | No Homology to Known | | | |
| | | | | Proteins | | | |
| 462 | Gma.8514.1.S1_at | 0.375 | CA851666 | Protein Destination & | | | Glutathione S-transferase |
| | | | | Storage | | | |
| 463 | Gma.4394.1.S1_a_at | 0.375 | CD409273 | Transcription | Transcription | Homeodomain | Transcription factor HEX, contains HOX |
| | | | | | Factor | | and HALZ domains |
| 464 | Gma.11269.1.S1_at | 0.374 | CD395046 | Transporter | | | |
| 465 | GmaAffx.71711.1.S1_at | 0.374 | AI988272 | No Homology to Known | | | |
| | | | | Proteins | | | |
| 466 | GmaAffx.8712.1.S1_s_at | 0.373 | AW349107 | Disease & Defense | | | |
| 467 | GmaAffx.90812.1.S1_at | 0.373 | CF806537 | Disease & Defense | | | |
| 468 | GmaAffx.19118.1.S1_at | 0.372 | BM523731 | Unclassified - | | | |
| | | | | Hypothetical Protein | | | |
| | | | | NOT Supported by cDNA | | | |
| 469 | Gma.398.1.A1_at | 0.372 | AW350679 | Metabolism | | | Homocysteine S-methyltransferase |
| 470 | Gma.5294.1.S1_at | 0.372 | BE658252 | Unclassified - | | | |
| | | | | Hypothetical Protein | | | |
| | | | | Supported by cDNA | | | |
| 471 | GmaAffx.78163.1.S1_s_at | 0.371 | BM520857 | Signal Transduction | Protein | | Serine/threonine protein phosphatase |
| | | | | | Phosphatase | | |
| 472 | Gma.3450.1.S1_at | 0.37 | BE657895 | Unclassified - | | | |
| | | | | Hypothetical Protein | | | |
| | | | | Supported by cDNA | | | |
| 473 | Gma.9259.1.S1_at | 0.37 | CA782161 | Transporter | | | |
| 474 | GmaAffx.90069.1.S1_s_at | 0.369 | CF805794 | Unclassified - | | | Leucine rich repeat proteins, some proteins |
| | | | | Hypothetical Protein | | | contain F-box |
| | | | | Supported by cDNA | | | |
| 475 | GmaAffx.91037.1.S1_s_at | 0.367 | CF806762 | Secondary Metabolism | | | Methyltransferase |
| 476 | Gma.16910.1.S1_at | 0.367 | BU549219 | Unclassified - Protein | | | |
| | | | | with Unknown Function | | | |
| 477 | GmaAffx.93183.1.S1_s_at | 0.365 | CF808908 | Secondary Metabolism | | | S-adenosylmethionine decarboxylase |
| 478 | Gma.11166.1.S1_s_at | 0.36 | AW705829 | Metabolism | | | |
| 479 | GmaAffx.81999.1.S1_at | 0.36 | AI416637 | Transcription | Transcription | Homeodomain | Transcription factor MEIS1 and related |
| | | | | | Factor | | HOX domain proteins |
| 480 | Gma.5725.1.S1_at | 0.36 | BM886367 | Disease & Defense | | | |
| 481 | GmaAffx.64261.1.A1_at | 0.359 | BI969080 | Unclassified - Protein | | | 1,4-benzoquinone reductase-like; Trp |
| | | | | with Unknown Function | | | repressor binding protein-like/protoplast- |
| | | | | | | | secreted protein |
| 482 | Gma.1951.2.S1_a_at | 0.358 | CA783485 | Protein Destination & | | | Serine carboxypeptidases |
| | | | | Storage | | | |
| 483 | GmaAffx.92127.1.S1_s_at | 0.358 | CF807852 | Transcription | Transcription | AP2/EREBP | |
| | | | | | Factor | | |
| 484 | Gma.3901.1.S1_at | 0.358 | CD394788 | Protein Destination & | Storage | | |
| | | | | Storage | Protein | | |
| 485 | Gma.8240.1.S1_at | 0.357 | BM092761 | Secondary Metabolism | | | Iron/ascorbate family oxidoreductases |
| 486 | Gma.13845.1.A1_at | 0.355 | CD407676 | Unclassified - | | | |
| | | | | Hypothetical Protein | | | |
| | | | | Supported by cDNA | | | |
| 487 | GmaAffx.1620.2.S1_at | 0.355 | BF324548 | Unclassified - | | | |
| | | | | Hypothetical Protein | | | |
| | | | | NOT Supported by cDNA | | | |
| 488 | GmaAffx.53845.1.S1_at | 0.354 | BG406552 | Transporter | | | Sulfate/bicarbonate/oxalate exchanger SAT- |
| | | | | | | | 1 and related transporters (SLC26 family) |
| 489 | GmaAffx.92166.1.S1_s_at | 0.353 | CF806313 | Signal Transduction | Unclassified | | Aspartyl protease |
| 490 | Gma.1314.1.S1_at | 0.353 | U63726.1 | Metabolism | | | Gamma-glutamyl hydrolase |
| 491 | GmaAffx.32606.1.S1_at | 0.353 | BM270707 | Unclassified - | | | Aquaporin (major intrinsic protein family) |
| | | | | Hypothetical Protein | | | |
| | | | | NOT Supported by cDNA | | | |
| 492 | Gma.790.1.A1_s_at | 0.351 | BE820450 | Unclassified - | | | Predicted E3 ubiquitin ligase |
| | | | | Hypothetical Protein | | | |
| | | | | Supported by cDNA | | | |
| 493 | GmaAffx.15471.3.S1_x_at | 0.351 | BE346777 | Transcription | Transcription | MYB | Transcription factor, Myb superfamily |
| | | | | | Factor | | |
| 494 | Gma.7227.2.S1_a_at | 0.349 | AW759183 | Protein Destination & | | | Ubiquitin-protein ligase |
| | | | | Storage | | | |
| 495 | Gma.3578.1.S1_at | 0.349 | AW156522 | Disease & Defense | | | Acyl-CoA synthetase |
| 496 | GmaAffx.47806.1.A1_at | 0.349 | BG508458 | No Homology to Known | | | |
| | | | | Proteins | | | |
| 497 | GmaAffx.75459.1.S1_s_at | 0.348 | BE348034 | Protein Destination & | Storage | | |
| | | | | Storage | Protein | | |
| 498 | GmaAffx.69198.1.S1_at | 0.347 | BM526951 | Unclassified - | | | Leucine rich repeat proteins, some proteins |
| | | | | Hypothetical Protein | | | contain F-box |
| | | | | Supported by cDNA | | | |
| 499 | GmaAffx.88945.1.S1_at | 0.347 | CK605738 | Unclassified - Protein | | | |
| | | | | with Unknown Function | | | |
| 500 | Gma.7454.1.S1_a_at | 0.345 | CD405901 | Transcription | Transcription | AUX/IAA | |
| | | | | | Factor | | |
| 501 | Gma.1326.1.S1_at | 0.345 | CD397515 | Secondary Metabolism | | | |
| 502 | GmaAffx.78768.1.S1_s_at | 0.344 | CA784898 | No Homology to Known | | | |
| | | | | Proteins | | | |
| 503 | GmaAffx.69369.1.S1_at | 0.343 | BG511072 | Unclassified - | | | Galactosyltransferases |
| | | | | Hypothetical Protein | | | |
| | | | | Supported by cDNA | | | |
| 504 | GmaAffx.82936.1.S1_at | 0.342 | AI938364 | Unclassified - Protein | | | |
| | | | | with Unknown Function | | | |
| 505 | GmaAffx.58606.1.S1_at | 0.341 | BM526502 | Metabolism | | | |
| 506 | Gma.10892.1.S1_a_at | 0.341 | BG839570 | Metabolism | | | Predicted carbonic anhydrase involved in |
| | | | | | | | protection against oxidative damage |
| 507 | Gma.17806.1.S1_at | 0.34 | BE820775 | Transporter | | | H+/oligopeptide symporter |
| 508 | GmaAffx.52114.1.S1_at | 0.339 | CA852143 | Metabolism | | | Heme oxygenase |
| 509 | GmaAffx.36470.1.A1_at | 0.337 | BU550552 | Unclassified - | | | |
| | | | | Hypothetical Protein | | | |
| | | | | Supported by cDNA | | | |
| 510 | GmaAffx.78768.2.S1_at | 0.335 | AI930773 | Unclassified - | | | |
| | | | | Hypothetical Protein | | | |
| | | | | Supported by cDNA | | | |
| 511 | Gma.1263.1.S1_at | 0.334 | AY144180.1 | Metabolism | | | Ca2+/calmodulin-dependent protein kinase, |
| | | | | | | | EF-Hand protein superfamily |
| 512 | Gma.17688.1.S1_s_at | 0.331 | CA937360 | Transcription | Transcription | Basic Helix- | |
| | | | | | Factor | Loop-Helix | |
| | | | | | | (bHLH) | |
| 513 | GmaAffx.11242.1.S1_s_at | 0.329 | AW317461 | Protein Destination & | | | Serine carboxypeptidases |
| | | | | Storage | | | |
| 514 | GmaAffx.86844.1.S1_at | 0.329 | BE822146 | Unclassified - Protein | | | |
| | | | | with Unknown Function | | | |
| 515 | Gma.398.2.S1_at | 0.328 | BU925900 | Metabolism | | | Homocysteine S-methyltransferase |
| 516 | Gma.221.1.S1_at | 0.328 | U41475.1 | Metabolism | | | Phosphoinositide-specific phospholipase C |
| 517 | Gma.12987.1.S1_at | 0.327 | CD392747 | Transcription | Transcription | Basic Helix- | |
| | | | | | Factor | Loop-Helix | |
| | | | | | | (bHLH) | |
| 518 | GmaAffx.50980.1.S1_s_at | 0.327 | BE823095 | Signal Transduction | Protein | | |
| | | | | | Phosphatase | | |
| 519 | GmaAffx.17892.1.S1_at | 0.326 | AI442822 | Unclassified - | | | |
| | | | | Hypothetical Protein | | | |
| | | | | Supported by cDNA | | | |
| 520 | GmaAffx.33121.1.S1_at | 0.325 | BQ611246 | Unclassified - | | | |
| | | | | Hypothetical Protein | | | |
| | | | | Supported by cDNA | | | |
| 521 | GmaAffx.77832.1.S1_at | 0.323 | BI971626 | Unclassified - | | | |
| | | | | Hypothetical Protein | | | |
| | | | | NOT Supported by cDNA | | | |
| 522 | GmaAffx.32011.2.S1_at | 0.322 | BM084966 | Transcription | Transcription | Basic Leucine | |
| | | | | | Factor | Zipper (bZIP) | |
| 523 | Gma.7437.1.S1_at | 0.322 | BU547705 | Unclassified - | | | |
| | | | | Hypothetical Protein | | | |
| | | | | Supported by cDNA | | | |
| 524 | GmaAffx.18113.2.S1_s_at | 0.321 | AW706795 | Unclassified - Protein | Hormone | | |
| | | | | with Unknown Function | Induced | | |
| 525 | Gma.17516.1.S1_at | 0.321 | CD396417 | Transporter | | | Ca2+/H+ antiporter VCX1 and related |
| | | | | | | | proteins |
| 526 | Gma.5889.1.S1_at | 0.32 | BQ610202 | Unclassified - | | | Endoplasmic reticulum protein EP58, |
| | | | | Hypothetical Protein | | | contains filamin rod domain and KDEL |
| | | | | NOT Supported by cDNA | | | motif |
| 527 | GmaAffx.80188.1.A1_at | 0.317 | AW349832 | No Homology to Known | | | |
| | | | | Proteins | | | |
| 528 | GmaAffx.23292.1.A1_at | 0.317 | BI967795 | No Homology to Known | | | |
| | | | | Proteins | | | |
| 529 | Gma.13030.1.S1_a_at | 0.316 | AI748012 | Protein Destination & | | | |
| | | | | Storage | | | |
| 530 | Gma.12731.2.A1_s_at | 0.315 | CD395848 | Unclassified - | | | Aquaporin (major intrinsic protein family) |
| | | | | Hypothetical Protein | | | |
| | | | | NOT Supported by cDNA | | | |
| 531 | GmaAffx.91312.1.S1_s_at | 0.314 | CF806002 | Transcription | Transcription | Homeodomain | Transcription factor HEX, contains HOX |
| | | | | | Factor | | and HALZ domains |
| 532 | Gma.1058.1.S1_at | 0.314 | BE820580 | Metabolism | | | |
| 533 | Gma.9647.1.S1_at | 0.313 | BE820997 | Cell Structure | | | O-methyltransferase |
| 534 | Gma.6033.1.S1_at | 0.312 | BI967767 | Unclassified - | | | |
| | | | | Hypothetical Protein | | | |
| | | | | Supported by cDNA | | | |
| 535 | Gma.17688.1.S1_at | 0.311 | CA937360 | Transcription | Transcription | Basic Helix- | |
| | | | | | Factor | Loop-Helix | |
| | | | | | | (bHLH) | |
| 536 | Gma.6129.2.S1_at | 0.31 | AW186006 | Protein Synthesis | | | Translation initiation factor 5A (elF-5A) |
| 537 | GmaAffx.46103.1.S1_at | 0.309 | BM091933 | Metabolism | | | Hydroxymethylglutaryl-CoA lyase |
| 538 | Gma.9365.1.A1_at | 0.309 | CD418433 | No Homology to Known | | | |
| | | | | Proteins | | | |
| 539 | GmaAffx.40923.1.S1_at | 0.308 | BG653848 | Disease & Defense | | | Selenium-binding protein |
| 540 | Gma.13273.1.A1_at | 0.306 | CD397107 | Unclassified - | | | |
| | | | | Hypothetical Protein | | | |
| | | | | Supported by cDNA | | | |
| 541 | GmaAffx.64967.1.S1_at | 0.306 | BU546115 | Secondary Metabolism | | | Iron/ascorbate family oxidoreductases |
| 542 | Gma.10969.2.S1_at | 0.302 | CD402329 | Unclassified - | | | Predicted lipase |
| | | | | Hypothetical Protein | | | |
| | | | | NOT Supported by cDNA | | | |
| 543 | GmaAffx.17918.1.S1_at | 0.301 | BG157024 | No Homology to Known | | | |
| | | | | Proteins | | | |
| 544 | GmaAffx.64807.1.S1_at | 0.301 | AW201972 | No Homology to Known | | | |
| | | | | Proteins | | | |
| 545 | GmaAffx.87793.2.S1_at | 0.301 | BQ740671 | Signal Transduction | Protein Kinase | | Glycogen synthase kinase-3 |
| 546 | Gma.11166.1.S1_x_at | 0.3 | AW705829 | Metabolism | | | |
| 547 | GmaAffx.45036.1.S1_at | 0.3 | BF595470 | No Homology to Known | | | |
| | | | | Proteins | | | |
| 548 | Gma.11205.1.S1_a_at | 0.299 | BF066553 | Metabolism | | | Transketolase |
| 549 | Gma.821.1.S1_at | 0.299 | AF243371.1 | Metabolism | | | Glutathione S-transferase |
| 550 | Gma.1619.1.S1_at | 0.298 | AW472195 | Unclassified - | | | |
| | | | | Hypothetical Protein | | | |
| | | | | Supported by cDNA | | | |
| 551 | Gma.1773.1.A1_at | 0.298 | CD404050 | Transcription | Transcription | Basic Helix- | |
| | | | | | Factor | Loop-Helix | |
| | | | | | | (bHLH) | |
| 552 | Gma.5468.2.S1_at | 0.298 | BE802025 | Metabolism | | | Beta-galactosidase |
| 553 | GmaAffx.34798.1.S1_at | 0.297 | BM143105 | Unclassified - | | | |
| | | | | Hypothetical Protein | | | |
| | | | | Supported by cDNA | | | |
| 554 | GmaAffx.89697.1.S1_s_at | 0.296 | CK606333 | Disease & Defense | | | |
| 555 | GmaAffx.81692.1.S1_at | 0.294 | CA937041 | No Homology to Known | | | |
| | | | | Proteins | | | |
| 556 | Gma.4990.1.A1_at | 0.294 | BI315863 | Unclassified - | | | Uncharacterized conserved protein |
| | | | | Hypothetical Protein | | | |
| | | | | Supported by cDNA | | | |
| 557 | GmaAffx.6790.1.S1_at | 0.294 | AI856683 | Disease & Defense | | | Soluble epoxide hydrolase |
| 558 | GmaAffx.83715.1.S1_at | 0.294 | BI785710 | Transporter | | | Glutaredoxin-related protein |
| 559 | GmaAffx.91428.1.A1_at | 0.293 | CF807137 | Metabolism | | | |
| 560 | GmaAffx.487.1.S1_at | 0.293 | BG511495 | Transporter | | | Predicted transporter (major facilitator |
| | | | | | | | superfamily) |
| 561 | Gma.5586.2.S1_at | 0.293 | CD398384 | Transcription | Transcription | Unclassified | CCAAT-binding factor, subunit B (HAP2) |
| | | | | | Factor | | |
| 562 | GmaAffx.86020.1.S1_at | 0.293 | BI701699 | Signal Transduction | Protein Kinase | AtCIPKs | Serine/threonine protein kinase |
| 563 | GmaAffx.24451.1.A1_at | 0.292 | BU550405 | Metabolism | Cytochrome | CYP90C | Cytochrome P450 CYP4/CYP19/CYP26 |
| | | | | | P450 | | subfamilies |
| 564 | Gma.1951.1.S1_at | 0.292 | BQ079657 | Protein Destination & | | | Serine carboxypeptidases |
| | | | | Storage | | | |
| 565 | Gma.1715.1.S1_at | 0.29 | BE191618 | Metabolism | | | |
| 566 | Gma.1573.2.S1_a_at | 0.286 | BI974098 | Metabolism | | | |
| 567 | GmaAffx.60673.1.S1_at | 0.286 | BU080770 | Unclassified - | | | |
| | | | | Hypothetical Protein | | | |
| | | | | NOT Supported by cDNA | | | |
| 568 | Gma.11873.1.S1_at | 0.283 | CA819977 | Unclassified - Protein | | | |
| | | | | with Unknown Function | | | |
| 569 | Gma.3617.1.S1_at | 0.282 | BE659297 | Cell Growth & Division | | | |
| 570 | GmaAffx.74438.1.S1_at | 0.282 | BE440325 | Unclassified - | | | |
| | | | | Hypothetical Protein | | | |
| | | | | NOT Supported by cDNA | | | |
| 571 | GmaAffx.18113.2.S1_x_at | 0.282 | AW706795 | Unclassified - Protein | Hormone | | |
| | | | | with Unknown Function | Induced | | |
| 572 | GmaAffx.6.1.S1_at | 0.276 | AW348562 | No Homology to Known | | | |
| | | | | Proteins | | | |
| 573 | Gma.8058.1.A1_at | 0.276 | BM271479 | Signal Transduction | Protein | | Serine/threonine protein phosphatase |
| | | | | | Phosphatase | | |
| 574 | Gma.1768.1.S1_s_at | 0.276 | BE820777 | Unclassified - | | | |
| | | | | Hypothetical Protein | | | |
| | | | | Supported by cDNA | | | |
| 575 | Gma.5701.2.S1_at | 0.275 | BI701059 | Unclassified - | | | |
| | | | | Hypothetical Protein | | | |
| | | | | Supported by cDNA | | | |
| 576 | GmaAffx.90395.1.S1_s_at | 0.275 | CF806120 | Unclassified - Protein | | | |
| | | | | with Unknown Function | | | |
| 577 | GmaAffx.17892.2.S1_at | 0.273 | BQ628677 | No Homology to Known | | | |
| | | | | Proteins | | | |
| 578 | GmaAffx.93591.1.S1_s_at | 0.27 | CF809316 | Metabolism | | | |
| 579 | Gma.10520.1.A1_at | 0.27 | CD398227 | Unclassified - | | | |
| | | | | Hypothetical Protein | | | |
| | | | | Supported by cDNA | | | |
| 580 | Gma.11026.2.S1_s_at | 0.27 | BQ080041 | Disease & Defense | | | |
| 581 | GmaAffx.84424.1.S1_at | 0.27 | AW311543 | Metabolism | | | Alcohol dehydrogenase, class V |
| 582 | Gma.1394.2.S1_a_at | 0.269 | BI470370 | Metabolism | | | |
| 583 | Gma.6192.1.S1_s_at | 0.269 | AW396773 | Metabolism | | | |
| 584 | GmaAffx.40114.1.S1_at | 0.267 | BI973325 | Unclassified - | | | |
| | | | | Hypothetical Protein | | | |
| | | | | Supported by cDNA | | | |
| 585 | Gma.6763.1.A1_at | 0.266 | BU544794 | Energy | | | Ferric reductase, NADH/NADPH oxidase |
| | | | | | | | and related proteins |
| 586 | Gma.10581.1.S1_at | 0.262 | AW396439 | Unclassified - | | | Predicted haloacid dehalogenase-like |
| | | | | Hypothetical Protein | | | hydrolase |
| | | | | Supported by cDNA | | | |
| 587 | Gma.1394.1.S1_at | 0.261 | BG839300 | Metabolism | | | |
| 588 | Gma.15487.1.S1_at | 0.259 | D63781.1 | Disease & Defense | | | Soluble epoxide hydrolase |
| 589 | GmaAffx.93592.1.S1_s_at | 0.259 | CF809317 | Unclassified - | | | |
| | | | | Hypothetical Protein | | | |
| | | | | Supported by cDNA | | | |
| 590 | Gma.4359.1.S1_at | 0.258 | AI794712 | Energy | | | Thioredoxin |
| 591 | GmaAffx.1620.1.S1_at | 0.257 | AW424105 | Unclassified - | | | |
| | | | | Hypothetical Protein | | | |
| | | | | NOT Supported by cDNA | | | |
| 592 | GmaAffx.1620.3.S1_s_at | 0.255 | AW132226 | No Homology to Known | | | |
| | | | | Proteins | | | |
| 593 | Gma.1980.1.A1_at | 0.255 | BM520466 | Signal Transduction | Protein Kinase | AtCIPKs | Serine/threonine protein kinase |
| 594 | GmaAffx.53324.1.A1_at | 0.254 | AW309251 | Transcription | Transcription | Basic Helix- | |
| | | | | | Factor | Loop-Helix | |
| | | | | | | (bHLH) | |
| 595 | Gma.3118.1.S1_s_at | 0.254 | BQ786048 | Disease & Defense | | | Predicted metallothionein |
| 596 | GmaAffx.77203.1.S1_at | 0.253 | BI970687 | Unclassified - | | | |
| | | | | Hypothetical Protein | | | |
| | | | | Supported by cDNA | | | |
| 597 | GmaAffx.52826.3.S1_at | 0.252 | BF596503 | Signal Transduction | Protein | | Serine/threonine protein phosphatase |
| | | | | | Phosphatase | | |
| 598 | GmaAffx.57432.1.S1_at | 0.25 | BE657443 | Unclassified - | | | |
| | | | | Hypothetical Protein | | | |
| | | | | NOT Supported by cDNA | | | |
| 599 | Gma.1394.2.S1_at | 0.249 | BI470370 | Metabolism | | | |
| 600 | Gma.15863.1.S1_s_at | 0.248 | CD413163 | Metabolism | | | |
| 601 | GmaAffx.93342.1.S1_s_at | 0.247 | CF809067 | Disease & Defense | | | Glutathione peroxidase |
| 602 | Gma.15093.1.S1_at | 0.247 | CD400374 | Protein Destination & | | | Glutathione S-transferase |
| | | | | Storage | | | |
| 603 | GmaAffx.91514.1.S1_at | 0.246 | CF807239 | No Homology to Known | | | |
| | | | | Proteins | | | |
| 604 | GmaAffx.90600.1.S1_at | 0.245 | CF806325 | Metabolism | | | |
| 605 | Gma.13672.1.A1_at | 0.242 | CD403249 | No Homology to Known | | | |
| | | | | Proteins | | | |
| 606 | Gma.17985.1.S1_at | 0.239 | CD416450 | Metabolism | | | |
| 607 | GmaAffx.90652.1.S1_at | 0.238 | CF806377 | Unclassified - | | | Predicted hydrolase/acyltransferase |
| | | | | Hypothetical Protein | | | (alpha/beta hydrolase superfamily) |
| | | | | Supported by cDNA | | | |
| 608 | Gma.8674.3.A1_at | 0.238 | BI469914 | Transporter | | | Predicted transporter (major facilitator |
| | | | | | | | superfamily) |
| 609 | Gma.13013.2.S1_a_at | 0.237 | AW457857 | Unclassified - | | | |
| | | | | Hypothetical Protein | | | |
| | | | | Supported by cDNA | | | |
| 610 | GmaAffx.89772.8.S1_at | 0.237 | CK605778 | Metabolism | | | |
| 611 | Gma.6684.1.S1_at | 0.236 | AW459859 | Signal Transduction | Unclassified | | Lanthionine synthetase C-like protein 1 |
| 612 | Gma.16500.1.S1_at | 0.235 | BE059054 | Metabolism | | | |
| 613 | GmaAffx.15196.1.S1_at | 0.234 | BU549436 | Metabolism | | | Long-chain acyl-CoA synthetases (AMP- |
| | | | | | | | forming) |
| 614 | GmaAffx.58712.1.S1_at | 0.233 | BF325034 | Transporter | | | Transporter, ABC superfamily (Breast |
| | | | | | | | cancer resistance protein) |
| 615 | GmaAffx.47288.1.S1_at | 0.233 | BU764979 | Unclassified - | | | GTPase Rab18, small G protein |
| | | | | Hypothetical Protein | | | superfamily |
| | | | | Supported by cDNA | | | |
| 616 | GmaAffx.40015.1.S1_at | 0.23 | BE609673 | No Homology to Known | | | |
| | | | | Proteins | | | |
| 617 | Gma.2086.1.S1_at | 0.228 | AF035671.1 | Metabolism | Amino Acid | | Arginase family protein |
| | | | | | Biosynthesis/ | | |
| | | | | | Metabolism | | |
| 618 | GmaAffx.15983.2.S1_s_at | 0.228 | AW279540 | Signal Transduction | Protein | | Serine/threonine protein phosphatase |
| | | | | | Phosphatase | | |
| 619 | GmaAffx.50980.1.S1_at | 0.227 | BE823095 | Signal Transduction | Protein | | |
| | | | | | Phosphatase | | |
| 620 | Gma.10827.3.S1_at | 0.227 | AW758966 | Secondary Metabolism | | | Iron/ascorbate family oxidoreductases |
| 621 | Gma.2252.1.S1_at | 0.227 | CD406598 | Unclassified - | | | |
| | | | | Hypothetical Protein | | | |
| | | | | Supported by cDNA | | | |
| 622 | GmaAffx.50980.1.S1_x_at | 0.225 | BE823095 | Signal Transduction | Protein | | |
| | | | | | Phosphatase | | |
| 623 | Gma.1527.2.S1_at | 0.223 | BE658017 | Secondary Metabolism | | | Flavonol reductase/cinnamoyl-CoA |
| | | | | | | | reductase |
| 624 | Gma.7006.1.S1_at | 0.223 | AF516880.1 | Cell Structure | | | |
| 625 | GmaAffx.15983.2.S1_at | 0.221 | AW279540 | Signal Transduction | Protein | | Serine/threonine protein phosphatase |
| | | | | | Phosphatase | | |
| 626 | Gma.3420.1.S1_at | 0.219 | CD406082 | Unclassified - | | | |
| | | | | Hypothetical Protein | | | |
| | | | | Supported by cDNA | | | |
| 627 | Gma.4252.1.S1_at | 0.216 | BE821208 | Metabolism | | | Glutamate decarboxylase and related |
| | | | | | | | proteins |
| 628 | GmaAffx.66215.1.S1_at | 0.215 | AI443109 | Signal Transduction | Unclassified | | Lanthionine synthetase C-like protein 1 |
| 629 | Gma.1979.1.S1_a_at | 0.213 | CD403280 | Unclassified - Protein | | | Multitransmembrane protein |
| | | | | with Unknown Function | | | |
| 630 | Gma.7824.1.S1_at | 0.212 | BU550492 | Metabolism | | | Kynurenine 3-monooxygenase and related |
| | | | | | | | flavoprotein monooxygenases |
| 631 | GmaAffx.15862.1.S1_at | 0.211 | BM092634 | Cell Structure | | | |
| 632 | GmaAffx.47788.1.A1_at | 0.21 | CD408318 | Metabolism | | | Aldehyde dehydrogenase |
| 633 | Gma.17985.2.S1_at | 0.209 | BE802263 | Metabolism | | | |
| 634 | Gma.968.1.S1_at | 0.209 | D28876.1 | Protein Destination & | | | Asparaginyl peptidases |
| | | | | Storage | | | |
| 635 | GmaAffx.38555.2.S1_at | 0.208 | BI425873 | No Homology to Known | | | |
| | | | | Proteins | | | |
| 636 | GmaAffx.79283.1.S1_at | 0.208 | CA819076 | Unclassified - | | | |
| | | | | Hypothetical Protein | | | |
| | | | | NOT Supported by cDNA | | | |
| 637 | Gma.7580.1.S1_at | 0.208 | BE057763 | Unclassified - | | | |
| | | | | Hypothetical Protein | | | |
| | | | | Supported by cDNA | | | |
| 638 | Gma.17921.2.S1_s_at | 0.208 | BM085020 | Metabolism | Sucrose | | Glycosyltransferase |
| | | | | | Biosynthesis | | |
| 639 | GmaAffx.90600.1.S1_s_at | 0.207 | CF806325 | Metabolism | | | |
| 640 | GmaAffx.92964.1.S1_at | 0.206 | CF808689 | No Homology to Known | | | |
| | | | | Proteins | | | |
| 641 | GmaAffx.24413.1.A1_at | 0.205 | BU548368 | Metabolism | | | 5′-AMP-activated protein kinase, gamma |
| | | | | | | | subunit |
| 642 | Gma.11672.1.S1_at | 0.205 | BF425929 | Unclassified - | | | |
| | | | | Hypothetical Protein | | | |
| | | | | Supported by cDNA | | | |
| 643 | Gma.6333.2.S1_at | 0.202 | CD396361 | No Homology to Known | | | |
| | | | | Proteins | | | |
| 644 | GmaAffx.93592.1.S1_x_at | 0.202 | CF809317 | Unclassified - | | | |
| | | | | Hypothetical Protein | | | |
| | | | | Supported by cDNA | | | |
| 645 | Gma.5469.1.S1_at | 0.202 | AW349200 | No Homology to Known | | | |
| | | | | Proteins | | | |
| 646 | Gma.1426.1.A1_at | 0.202 | AI443364 | Metabolism | | | |
| 647 | GmaAffx.11172.1.A1_at | 0.201 | CD404556 | No Homology to Known | | | |
| | | | | Proteins | | | |
| 648 | Gma.13150.1.A1_at | 0.2 | CD411577 | Unclassified - Protein | Hormone | | |
| | | | | with Unknown Function | Induced | | |
| 649 | Gma.13340.1.S1_at | 0.199 | BM523855 | Metabolism | | | UDP-glucuronosyl and UDP-glucosyl |
| | | | | | | | transferase |
| 650 | Gma.177.1.S1_at | 0.196 | AF004806.1 | Protein Destination & | Storage | | Reticulon |
| | | | | Storage | Protein | | |
| 651 | GmaAffx.87417.1.S1_at | 0.195 | BM568072 | Metabolism | | | |
| 652 | GmaAffx.64794.1.S1_at | 0.192 | CA801749 | Unclassified - | | | |
| | | | | Hypothetical Protein | | | |
| | | | | Supported by cDNA | | | |
| 653 | Gma.12068.1.A1_at | 0.192 | BU547739 | Transporter | | | Transporter, ABC superfamily (Breast |
| | | | | | | | cancer resistance protein) |
| 654 | Gma.6921.1.S1_at | 0.188 | BE823209 | Signal Transduction | Unclassified | | Phosphatidylethanolamine binding protein |
| 655 | Gma.12227.1.S1_at | 0.186 | BG511671 | Unclassified - | | | |
| | | | | Hypothetical Protein | | | |
| | | | | NOT Supported by cDNA | | | |
| 656 | GmaAffx.12665.1.A1_at | 0.186 | BE822073 | Metabolism | | | |
| 657 | Gma.6192.1.S1_at | 0.183 | AW396773 | Metabolism | | | |
| 658 | GmaAffx.45957.2.S1_at | 0.181 | BI974135 | No Homology to Known | | | |
| | | | | Proteins | | | |
| 659 | GmaAffx.93220.1.S1_at | 0.18 | CF808945 | No Homology to Known | | | |
| | | | | Proteins | | | |
| 660 | GmaAffx.51466.1.A1_at | 0.179 | CD406245 | Metabolism | | | UDP-glucuronosyl and UDP-glucosyl |
| | | | | | | | transferase |
| 661 | GmaAffx.4968.1.S1_at | 0.178 | BI967691 | Unclassified - | | | |
| | | | | Hypothetical Protein | | | |
| | | | | Supported by cDNA | | | |
| 662 | Gma.13062.1.S1_at | 0.176 | CD394315 | Metabolism | | | |
| 663 | GmaAffx.89772.8.S1_x_at | 0.174 | CK605778 | Metabolism | | | |
| 664 | Gma.1495.1.S1_at | 0.174 | BI973545 | No Homology to Known | | | |
| | | | | Proteins | | | |
| 665 | GmaAffx.48718.1.S1_at | 0.173 | BM528538 | No Homology to Known | | | |
| | | | | Proteins | | | |
| 666 | Gma.2382.1.S1_x_at | 0.171 | BG789835 | Metabolism | | | |
| 667 | Gma.5331.1.S1_a_at | 0.167 | BI892702 | Transcription | Transcription | NAC Domain | |
| | | | | | Factor | | |
| 668 | Gma.2540.2.S1_s_at | 0.165 | BI787366 | No Homology to Known | | | |
| | | | | Proteins | | | |
| 669 | GmaAffx.92167.1.A1_s_at | 0.164 | CF807892 | Unclassified - | | | |
| | | | | Hypothetical Protein | | | |
| | | | | NOT Supported by cDNA | | | |
| 670 | GmaAffx.31862.1.S1_at | 0.163 | BE822501 | Unclassified - | | | |
| | | | | Hypothetical Protein | | | |
| | | | | Supported by cDNA | | | |
| 671 | Gma.8022.1.S1_at | 0.161 | BQ628998 | Disease & Defense | | | Soluble epoxide hydrolase |
| 672 | Gma.15.1.S1_at | 0.161 | U66316.1 | Unclassified - Protein | | | |
| | | | | with Unknown Function | | | |
| 673 | Gma.1527.1.S1_x_at | 0.16 | BI968185 | Secondary Metabolism | | | Flavonol reductase/cinnamoyl-CoA |
| | | | | | | | reductase |
| 674 | Gma.10615.1.A1_at | 0.156 | BU545626 | Transcription | Transcription | Basic Helix- | |
| | | | | | Factor | Loop-Helix | |
| | | | | | | (bHLH) | |
| 675 | GmaAffx.93116.1.S1_at | 0.15 | CF806290 | Disease & Defense | | | |
| 676 | GmaAffx.71308.1.S1_at | 0.15 | BF595112 | Transcription | Transcription | Heat Shock | Heat shock transcription factor |
| | | | | | Factor | | |
| 677 | Gma.1527.1.S1_a_at | 0.148 | BI968185 | Secondary Metabolism | | | Flavonol reductase/cinnamoyl-CoA |
| | | | | | | | reductase |
| 678 | GmaAffx.80393.1.A1_at | 0.144 | BE658680 | No Homology to Known | | | |
| | | | | Proteins | | | |
| 679 | Gma.12094.1.A1_at | 0.144 | CD395088 | Unclassified - | | | |
| | | | | Hypothetical Protein | | | |
| | | | | Supported by cDNA | | | |
| 680 | Gma.857.1.S1_at | 0.143 | BE822264 | No Homology to Known | | | |
| | | | | Proteins | | | |
| 681 | Gma.16477.1.S1_at | 0.142 | BE824134 | No Homology to Known | | | |
| | | | | Proteins | | | |
| 682 | Gma.4837.1.S1_at | 0.14 | BI969343 | Transporter | | | H+/oligopeptide symporter |
| 683 | Gma.728.1.A1_at | 0.139 | BE658779 | No Homology to Known | | | |
| | | | | Proteins | | | |
| 684 | Gma.11026.1.S1_at | 0.139 | AW756869 | Disease & Defense | | | |
| 685 | Gma.5331.1.S1_at | 0.131 | BI892702 | Transcription | Transcription | NAC Domain | |
| | | | | | Factor | | |
| 686 | Gma.16566.1.S1_at | 0.131 | CD400974 | Metabolism | | | Fumarase |
| 687 | Gma.13035.1.S1_at | 0.13 | CD396536 | No Homology to Known | | | |
| | | | | Proteins | | | |
| 688 | GmaAffx.57432.2.S1_at | 0.13 | CA819434 | No Homology to Known | | | |
| | | | | Proteins | | | |
| 689 | GmaAffx.89624.1.A1_s_at | 0.124 | CK606100 | Transcription | Transcription | Basic Leucine | |
| | | | | | Factor | Zipper (bZIP) | |
| 690 | Gma.10999.1.S1_at | 0.124 | CA850772 | Disease & Defense | | | |
| 691 | GmaAffx.91338.1.S1_at | 0.123 | CF807063 | Unclassified - | | | Predicted haloacid dehalogenase-like |
| | | | | Hypothetical Protein | | | hydrolase |
| | | | | Supported by cDNA | | | |
| 692 | GmaAffx.71222.1.S1_at | 0.121 | AW460050 | Metabolism | | | 5′-AMP-activated protein kinase, gamma |
| | | | | | | | subunit |
| 693 | GmaAffx.73698.1.S1_at | 0.118 | BU964780 | Metabolism | | | 5′-AMP-activated protein kinase, gamma |
| | | | | | | | subunit |
| 694 | GmaAffx.88546.1.S1_at | 0.114 | BE801134 | Metabolism | | | |
| 695 | GmaAffx.89407.1.A1_s_at | 0.11 | CK606273 | Transporter | | | |
| 696 | Gma.12684.1.S1_at | 0.107 | CD394773 | Transcription | Transcription | Zinc Finger | |
| | | | | | Factor | | |
| 697 | Gma.11993.1.S1_at | 0.107 | BU551348 | Transporter | | | Transporter, ABC superfamily (Breast |
| | | | | | | | cancer resistance protein) |
| 698 | Gma.2382.1.S1_s_at | 0.107 | BG789835 | Metabolism | | | |
| 699 | GmaAffx.71308.2.A1_at | 0.105 | AW569888 | No Homology to Known | | | |
| | | | | Proteins | | | |
| 700 | Gma.1649.1.A1_at | 0.104 | BE659535 | No Homology to Known | | | |
| | | | | Proteins | | | |
| 701 | GmaAffx.26908.1.S1_at | 0.0998 | BF425834 | Secondary Metabolism | | | Beta, beta-carotene 15,15′-dioxygenase and |
| | | | | | | | related enzymes |
| 702 | GmaAffx.72198.1.A1_at | 0.099 | BE823481 | Metabolism | | | UDP-glucuronosyl and UDP-glucosyl |
| | | | | | | | transferase |
| 703 | GmaAffx.66301.1.S1_at | 0.0974 | BG510223 | Secondary Metabolism | Ethylene | | Iron/ascorbate family oxidoreductases |
| | | | | | Biosynthesis | | |
| 704 | GmaAffx.30792.1.A1_at | 0.0973 | BU544815 | Unclassified - | | | |
| | | | | Hypothetical Protein | | | |
| | | | | Supported by cDNA | | | |
| 705 | GmaAffx.11049.1.S1_at | 0.0954 | BU545140 | Unclassified - Protein | | | Phosphatidylethanolamine binding protein |
| | | | | with Unknown Function | | | |
| 706 | GmaAffx.3369.2.A1_at | 0.0914 | BU550316 | Cell Structure | | | |
| 707 | GmaAffx.91479.1.S1_s_at | 0.0865 | CF807204 | No Homology to Known | | | |
| | | | | Proteins | | | |
| 708 | GmaAffx.93212.1.S1_s_at | 0.0863 | CF807524 | Transporter | | | |
| 709 | Gma.8530.1.S1_at | 0.086 | AF109720.1 | Protein Destination & | Storage | | |
| | | | | Storage | Protein | | |
| 710 | Gma.10896.3.A1_at | 0.0833 | BI316837 | Metabolism | | | Aldehyde dehydrogenase |
| 711 | GmaAffx.42732.1.S1_at | 0.0824 | BU765857 | Protein Destination & | Ubiquitin | | FOG: Predicted E3 ubiquitin ligase |
| | | | | Storage | Degradation | | |
| 712 | Gma.12393.3.S1_at | 0.0822 | BM085488 | Secondary Metabolism | | | Beta, beta-carotene 15,15′-dioxygenase and |
| | | | | | | | related enzymes |
| 713 | Gma.7831.1.S1_s_at | 0.0815 | CD393602 | Transporter | | | |
| 714 | Gma.15534.1.S1_at | 0.0813 | U50081.1 | Metabolism | | | |
| 715 | GmaAffx.10582.1.S1_at | 0.0788 | BM523687 | Transcription | Transcription | CCAAT | CCAAT-binding factor, subunit A (HAP3) |
| | | | | | Factor | | |
| 716 | GmaAffx.81851.1.S1_at | 0.0772 | AW706751 | Unclassified - | | | |
| | | | | Hypothetical Protein | | | |
| | | | | Supported by cDNA | | | |
| 717 | GmaAffx.26868.1.S1_at | 0.0752 | BF325443 | Unclassified - | | | Tomosyn and related SNARE-interacting |
| | | | | Hypothetical Protein | | | proteins |
| | | | | NOT Supported by cDNA | | | |
| 718 | GmaAffx.295.1.A1_s_at | 0.0741 | BG653267 | Protein Destination & | | | |
| | | | | Storage | | | |
| 719 | GmaAffx.12933.1.S1_at | 0.0738 | AW395787 | Transcription | Transcription | CCAAT | CCAAT-binding factor, subunit B (HAP2) |
| | | | | | Factor | | |
| 720 | Gma.2986.1.S1_at | 0.0737 | BM271324 | Metabolism | | | 5′-AMP-activated protein kinase, gamma |
| | | | | | | | subunit |
| 721 | GmaAffx.27331.1.S1_at | 0.0729 | BM520297 | Unclassified - | | | |
| | | | | Hypothetical Protein | | | |
| | | | | NOT Supported by cDNA | | | |
| 722 | GmaAffx.82263.1.S1_s_at | 0.068 | AW568454 | No Homology to Known | | | |
| | | | | Proteins | | | |
| 723 | GmaAffx.76276.1.S1_at | 0.0658 | BE331452 | Transporter | | | Copper chaperone |
| 724 | GmaAffx.81362.1.S1_at | 0.0651 | BF324988 | Cell Structure | | | |
| 725 | Gma.17326.1.A1_at | 0.0634 | AW568254 | Unclassified - | | | Permease of the major facilitator |
| | | | | Hypothetical Protein | | | superfamily |
| | | | | NOT Supported by cDNA | | | |
| 726 | Gma.8187.1.S1_a_at | 0.0628 | BU549133 | Signal Transduction | Protein | | Serine/threonine protein phosphatase |
| | | | | | Phosphatase | | |
| 727 | GmaAffx.9911.1.S1_at | 0.0603 | BU547808 | Unclassified - | | | Tomosyn and related SNARE-interacting |
| | | | | Hypothetical Protein | | | proteins |
| | | | | NOT Supported by cDNA | | | |
| 728 | Gma.1061.1.A1_at | 0.0594 | CD408183 | Metabolism | | | |
| 729 | Gma.13854.1.S1_at | 0.0534 | CD408166 | Metabolism | | | |
| 730 | Gma.8318.1.A1_at | 0.0533 | BU550469 | No Homology to Known | | | |
| | | | | Proteins | | | |
| 731 | GmaAffx.93212.1.S1_at | 0.0532 | CF807524 | Transporter | | | |
| 732 | Gma.5929.1.S1_at | 0.0517 | BQ610446 | No Homology to Known | | | |
| | | | | Proteins | | | |
| 733 | Gma.176.1.S1_s_at | 0.0488 | AF004810.1 | Protein Destination & | Storage | | |
| | | | | Storage | Protein | | |
| 734 | GmaAffx.93212.1.S1_x_at | 0.0475 | CF807524 | Transporter | | | |
| 735 | Gma.13296.3.S1_at | 0.0456 | CD418313 | Transporter | | | |
| 736 | GmaAffx.57970.1.S1_at | 0.0435 | BM886862 | Transcription | Transcription | NAC Domain | |
| | | | | | Factor | | |
| 737 | GmaAffx.75697.1.S1_s_at | 0.0435 | BU090518 | Unclassified - | | | |
| | | | | Hypothetical Protein | | | |
| | | | | Supported by cDNA | | | |
| 738 | GmaAffx.63464.1.S1_at | 0.0409 | BG156739 | No Homology to Known | | | |
| | | | | Proteins | | | |
| 739 | Gma.3774.2.S1_at | 0.0343 | CD393365 | Energy | | | Thioredoxin, nucleoredoxin and related |
| | | | | | | | proteins |
| 740 | Gma.5162.1.A1_at | 0.0327 | BI971156 | Signal Transduction | Protein Kinase | | |
| 741 | Gma.10830.1.S1_at | 0.0323 | BE820822 | Unclassified - | | | |
| | | | | Hypothetical Protein | | | |
| | | | | Supported by cDNA | | | |
| 742 | GmaAffx.10204.1.S1_at | 0.0297 | BQ298213 | Unclassified - | | | |
| | | | | Hypothetical Protein | | | |
| | | | | Supported by cDNA | | | |
| 743 | Gma.10904.1.S1_at | 0.0244 | CD408184 | Protein Destination & | | | |
| | | | | Storage | | | |
| 744 | Gma.3774.1.S1_at | 0.0206 | BU550698 | Energy | | | Thioredoxin, nucleoredoxin and related |
| | | | | | | | proteins |
| 745 | Gma.3774.2.S1_a_at | 0.0202 | CD393365 | Energy | | | Thioredoxin, nucleoredoxin and related |
| | | | | | | | proteins |
| 746 | Gma.9947.1.S1_at | 0.0195 | BF324269 | Unclassified - | | | |
| | | | | Hypothetical Protein | | | |
| | | | | Supported by cDNA | | | |
| 747 | Gma.2912.1.S1_s_at | 0.0193 | AF117884.1 | Protein Destination & | Storage | | Uncharacterized conserved protein |
| | | | | Storage | Protein | | |
| 748 | Gma.3000.1.S1_at | 0.0192 | BE657172 | No Homology to Known | | | |
| | | | | Proteins | | | |
| 749 | Gma.10904.1.S1_s_at | 0.0156 | CD408184 | Protein Destination & | | | |
| | | | | Storage | | | |
| 750 | GmaAffx.1785.1.S1_at | 0.0156 | BF325331 | Transcription | Transcription | Homeodomain | Transcription factor HEX, contains HOX |
| | | | | | Factor | | and HALZ domains |
| 751 | Gma.2912.1.S1_at | 0.0134 | AF117884.1 | Protein Destination & | Storage | | Uncharacterized conserved protein |
| | | | | Storage | Protein | | |
| 752 | Gma.3000.2.S1_a_at | 0.0111 | BM270844 | No Homology to Known | | | |
| | | | | Proteins | | | |
| 753 | GmaAffx.68621.1.A1_at | 0.00817 | BI893889 | Unclassified - Protein | Stress induced | | |
| | | | | with Unknown Function | | | |

TABLE 12

| | | Fold | | | | | |
|---|---|---|---|---|---|---|---|
| ID | Gene Name | Change | Soybean_GenBank_Acc | Functional Category | Sub-Family | Gene Family | Additional Description |
| 1 | Gma.5716.1.S1_at | 11.64 | BQ453925 | No Homology to Known | | | |
| | | | | Proteins | | | |
| 2 | GmaAffx.87679.1.S1_at | 9.533 | BM523734 | Metabolism | Trehalose | | Trehalose-6-phosphate synthase component |
| | | | | | Biosynthesis | | TPS1 and related subunits |
| 3 | GmaAffx.89567.1.A1_at | 9.001 | CK606157 | Signal Transduction | Calcium | Calcium | Ca2+/calmodulin-dependent protein kinase, EF- |
| | | | | | Binding | Dependent | Hand protein superfamily |
| | | | | | | Protein Kinase | |
| 4 | GmaAffx.87679.2.S1_s_at | 7.575 | AW102083 | Metabolism | Trehalose | | Trehalose-6-phosphate synthase component |
| | | | | | Biosynthesis | | TPS1 and related subunits |
| 5 | GmaAffx.9866.1.S1_at | 7.144 | BM524432 | Metabolism | | | |
| 6 | GmaAffx.9618.1.S1_at | 7.08 | BM092432 | No Homology to Known | | | |
| | | | | Proteins | | | |
| 7 | GmaAffx.89396.2.A1_s_at | 6.061 | CK605844 | Cell Structure | | | |
| 8 | GmaAffx.70875.1.S1_s_at | 6.049 | AW100693 | No Homology to Known | | | |
| | | | | Proteins | | | |
| 9 | GmaAffx.40860.2.S1_at | 5.75 | BG509282 | Disease & Defense | | | |
| 10 | Gma.152.1.S1_at | 5.694 | Y10492.1 | Metabolism | Cytochrome | CYP93D | Cytochrome P450 CYP2 subfamily |
| | | | | | P450 | | |
| 11 | Gma.17961.1.S1_at | 5.671 | AW350363 | Protein Destination & Storage | Storage Protein | | |
| 12 | GmaAffx.93541.2.S1_at | 5.572 | CF805889 | Protein Destination & Storage | | | Gelatinase A and related matrix |
| | | | | | | | metalloproteases |
| 13 | Gma.16425.2.S1_s_at | 5.528 | AW099501 | Unclassified - Hypothetical | | | Copper chaperone |
| | | | | Protein NOT Supported by | | | |
| | | | | cDNA | | | |
| 14 | Gma.11259.1.S1_at | 5.423 | AI442316 | Unclassified - Hypothetical | | | |
| | | | | Protein Supported by cDNA | | | |
| 15 | GmaAffx.2743.1.S1_s_at | 5.137 | BM568087 | Signal Transduction | Protein Kinase | | |
| 16 | Gma.5629.1.S1_at | 5.076 | BQ740550 | Disease & Defense | | | |
| 17 | GmaAffx.81930.1.S1_at | 5.036 | BG789998 | Unclassified - Hypothetical | | | |
| | | | | Protein Supported by cDNA | | | |
| 18 | GmaAffx.2743.1.S1_at | 5.014 | BM568087 | Signal Transduction | Protein Kinase | | |
| 19 | Gma.5483.1.S1_s_at | 4.957 | CD414581 | Transcription | Transcription | Basic Leucine | |
| | | | | | Factor | Zipper (bZIP) | |
| 20 | Gma.4226.2.S1_at | 4.938 | BM568170 | Unclassified - Hypothetical | | | |
| | | | | Protein Supported by cDNA | | | |
| 21 | Gma.3639.1.S1_at | 4.902 | AB083030.1 | Protein Destination & Storage | Storage Protein | | |
| 22 | GmaAffx.70509.1.A1_at | 4.887 | CD400633 | No Homology to Known | | | |
| | | | | Proteins | | | |
| 23 | Gma.1400.2.S1_at | 4.811 | BG510739 | Signal Transduction | Protein Kinase | AtCIPKs | Serine/threonine protein kinase |
| 24 | Gma.4071.1.S1_at | 4.771 | BG651692 | Unclassified - Hypothetical | | | |
| | | | | Protein Supported by cDNA | | | |
| 25 | GmaAffx.69888.1.A1_at | 4.769 | BU549275 | No Homology to Known | | | |
| | | | | Proteins | | | |
| 26 | GmaAffx.87872.1.S1_at | 4.606 | BM885036 | Metabolism | | | CTP synthase (UTP-ammonia lyase) |
| 27 | GmaAffx.18846.1.S1_at | 4.583 | BM091991 | Signal Transduction | Small GTPase | | FOG: WD40 repeat |
| 28 | GmaAffx.89158.1.A1_at | 4.344 | CK605958 | Intracellular Traffic | | | Phosphatidylinositol transfer protein SEC 14 |
| | | | | | | | and related proteins |
| 29 | Gma.16425.3.S1_s_at | 4.229 | AW508123 | Unclassified - Protein with | | | Copper chaperone |
| | | | | Unknown Function | | | |
| 30 | Gma.7474.1.A1_at | 4.206 | BU547968 | Unclassified - Hypothetical | | | |
| | | | | Protein Supported by cDNA | | | |
| 31 | Gma.16832.1.S1_s_at | 4.194 | L22029.1 | No Homology to Known | | | |
| | | | | Proteins | | | |
| 32 | GmaAffx.52494.1.S1_at | 4.174 | BE473800 | Unclassified - Hypothetical | | | |
| | | | | Protein NOT Supported by | | | |
| | | | | cDNA | | | |
| 33 | GmaAffx.48172.1.S1_at | 4.033 | BF009291 | Transporter | | | Predicted small molecule transporter |
| 34 | Gma.184.1.S1_at | 3.978 | AW101261 | Unclassified - Hypothetical | | | |
| | | | | Protein Supported by cDNA | | | |
| 35 | Gma.7726.1.A1_at | 3.929 | BM886413 | Unclassified - Protein with | | | Ca2+-binding protein (centrin/caltractin), EF- |
| | | | | Unknown Function | | | Hand superfamily protein |
| 36 | Gma.15889.1.S1_at | 3.928 | AW348133 | Transcription | Transcription | WRKY | |
| | | | | | Factor | | |
| 37 | Gma.2010.1.S1_a_at | 3.876 | CD402050 | Transcription | Transcription | Zinc Finger | GATA-4/5/6 transcription factors |
| | | | | | Factor | | |
| 38 | GmaAffx.55767.1.S1_at | 3.817 | BQ079515 | Signal Transduction | Calcium | | Calmodulin and related proteins (EF-Hand |
| | | | | | Binding | | superfamily) |
| 39 | GmaAffx.78720.2.S1_at | 3.816 | BI788325 | No Homology to Known | | | |
| | | | | Proteins | | | |
| 40 | Gma.6555.1.S1_at | 3.81 | BM954521 | Unclassified - Hypothetical | | | |
| | | | | Protein Supported by cDNA | | | |
| 41 | PsAffx.psHB044xM02f_at | 3.793 | | | | | |
| 42 | Gma.5631.1.S1_at | 3.748 | CD405214 | Transporter | | | Amino acid transporters |
| 43 | GmaAffx.24583.1.S1_at | 3.746 | BE609540 | Signal Transduction | Protein Kinase | | Serine/threonine protein kinase |
| 44 | Gma.2446.2.S1_at | 3.739 | BI427101 | Unclassified - Hypothetical | | | |
| | | | | Protein Supported by cDNA | | | |
| 45 | GmaAffx.93270.1.S1_x_at | 3.706 | CF808995 | No Homology to Known | | | |
| | | | | Proteins | | | |
| 46 | GmaAffx.78636.1.S1_at | 3.706 | BQ627652 | No Homology to Known | | | |
| | | | | Proteins | | | |
| 47 | GmaAffx.18649.1.S1_at | 3.693 | AW351025 | No Homology to Known | | | |
| | | | | Proteins | | | |
| 48 | GmaAffx.10340.1.S1_x_at | 3.668 | CA938421 | No Homology to Known | | | |
| | | | | Proteins | | | |
| 49 | Gma.13447.1.A1_at | 3.656 | CD398305 | No Homology to Known | | | |
| | | | | Proteins | | | |
| 50 | GmaAffx.9145.1.S1_s_at | 3.649 | BU763814 | Disease & Defense | | | |
| 51 | GmaAffx.65829.1.A1_at | 3.588 | CD392418 | Transcription | Transcription | AP2/EREBP | |
| | | | | | Factor | | |
| 52 | Gma.12665.2.A1_at | 3.529 | CD394142 | Unclassified - Hypothetical | | | |
| | | | | Protein Supported by cDNA | | | |
| 53 | Gma.14471.2.S1_at | 3.523 | CD408292 | Transporter | | | Aquaporin (major intrinsic protein family) |
| 54 | Gma.15889.1.S1_s_at | 3.503 | AW348133 | Transcription | Transcription | WRKY | |
| | | | | | Factor | | |
| 55 | Gma.2488.1.S1_at | 3.467 | CD406079 | Protein Destination & Storage | | | Serine carboxypeptidases (lysosomal cathepsin |
| | | | | | | | A) |
| 56 | Gma.5579.1.S1_at | 3.467 | AW119360 | Secondary Metabolism | Flavonoid | | |
| | | | | | Biosynthesis | | |
| 57 | GmaAffx.36514.1.S1_at | 3.452 | BE658341 | Disease & Defense | | | |
| 58 | GmaAffx.8098.1.S1_at | 3.437 | AW156410 | No Homology to Known | | | |
| | | | | Proteins | | | |
| 59 | GmaAffx.89512.1.A1_at | 3.403 | CK606530 | No Homology to Known | | | |
| | | | | Proteins | | | |
| 60 | GmaAffx.47815.1.A1_at | 3.374 | CD415065 | Unclassified - Hypothetical | | | |
| | | | | Protein NOT Supported by | | | |
| | | | | cDNA | | | |
| 61 | Gma.8896.1.S1_at | 3.369 | BQ252812 | Transcription | Transcription | AP2/EREBP | |
| | | | | | Factor | | |
| 62 | GmaAffx.18566.1.A1_at | 3.363 | BU550417 | Unclassified - Hypothetical | | | |
| | | | | Protein NOT Supported by | | | |
| | | | | cDNA | | | |
| 63 | GmaAffx.90960.1.A1_s_at | 3.356 | CF806685 | Signal Transduction | Protein Kinase | | |
| 64 | Gma.12665.1.S1_at | 3.331 | BE347113 | Unclassified - Hypothetical | | | |
| | | | | Protein Supported by cDNA | | | |
| 65 | Gma.340.1.S1_at | 3.327 | AW350127 | Cell Structure | | | |
| 66 | GmaAffx.85374.1.S1_at | 3.263 | BI317002 | Signal Transduction | Protein Kinase | | |
| 67 | GmaAffx.11276.1.S1_s_at | 3.252 | AW567677 | Unclassified - Hypothetical | | | |
| | | | | Protein Supported by cDNA | | | |
| 68 | GmaAffx.54265.1.S1_s_at | 3.24 | BU546096 | Signal Transduction | Protein Kinase | | |
| 69 | GmaAffx.37908.1.A1_at | 3.229 | BU546116 | Signal Transduction | Protein Kinase | | |
| 70 | Gma.12867.1.A1_at | 3.18 | CD393518 | Unclassified - Hypothetical | | | |
| | | | | Protein Supported by cDNA | | | |
| 71 | GmaAffx.10340.1.S1_at | 3.178 | CA938421 | No Homology to Known | | | |
| | | | | Proteins | | | |
| 72 | GmaAffx.78434.1.S1_at | 3.177 | BG041904 | Unclassified - Hypothetical | | | Predicted membrane protein, contains DoH |
| | | | | Protein NOT Supported by | | | and Cytochrome b-561/ferric reductase |
| | | | | cDNA | | | transmembrane domains |
| 73 | Gma.13433.1.A1_at | 3.173 | CD398049 | No Homology to Known | | | |
| | | | | Proteins | | | |
| 74 | Gma.5629.2.S1_a_at | 3.165 | BG042866 | Disease & Defense | | | |
| 75 | Gma.2972.1.S1_at | 3.158 | AW350602 | Unclassified - Hypothetical | | | Phosphatidylinositol transfer protein SEC 14 |
| | | | | Protein Supported by cDNA | | | and related proteins |
| 76 | GmaAffx.56563.1.S1_at | 3.133 | CA820002 | No Homology to Known | | | |
| | | | | Proteins | | | |
| 77 | Gma.368.1.S1_at | 3.124 | AW349594 | No Homology to Known | | | |
| | | | | Proteins | | | |
| 78 | Gma.16308.1.S1_s_at | 3.061 | U64916.1 | Unclassified - Hypothetical | | | Copper chaperone |
| | | | | Protein NOT Supported by | | | |
| | | | | cDNA | | | |
| 79 | GmaAffx.45530.1.S1_at | 3.048 | BG789917 | Signal Transduction | Protein Kinase | | |
| 80 | GmaAffx.11858.1.A1_s_at | 3.047 | AW351023 | Metabolism | Phospholipid | | Glycosylphosphatidylinositol-specific |
| | | | | | Biosynthesis | | phospholipase C |
| 81 | Gma.3777.1.S1_a_at | 3.008 | CD399954 | Unclassified - Hypothetical | | | |
| | | | | Protein Supported by cDNA | | | |
| 82 | GmaAffx.65419.1.S1_at | 3.003 | CD405955 | Signal Transduction | Protein Kinase | | |
| 83 | GmaAffx.40860.1.S1_at | 3.002 | BQ628787 | Disease & Defense | | | |
| 84 | Gma.8797.1.A1_at | 2.953 | BI969881 | Unclassified - Hypothetical | | | |
| | | | | Protein NOT Supported by | | | |
| | | | | cDNA | | | |
| 85 | GmaAffx.84266.1.S1_at | 2.952 | AI495798 | Signal Transduction | Protein Kinase | AtC1PKs | Serine/threonine protein kinase |
| 86 | GmaAffx.66979.1.S1_at | 2.944 | BI497616 | Signal Transduction | Protein Kinase | AtC1PKs | Serine/threonine protein kinase |
| 87 | GmaAffx.78720.1.S1_at | 2.93 | BM526650 | No Homology to Known | | | |
| | | | | Proteins | | | |
| 88 | GmaAffx.21211.1.S1_at | 2.919 | BU550606 | Protein Destination & Storage | | | Molecular chaperone (HSP90 family) |
| 89 | Gma.15495.1.A1_at | 2.918 | CD409942 | Unclassified - Hypothetical | | | Serine/threonine protein kinase |
| | | | | Protein NOT Supported by | | | |
| | | | | cDNA | | | |
| 90 | Gma.10334.1.S1_at | 2.918 | AW234479 | Unclassified - Hypothetical | | | |
| | | | | Protein NOT Supported by | | | |
| | | | | cDNA | | | |
| 91 | Gma.6302.1.S1_at | 2.916 | BI786195 | Cell Structure | | | Beta-glucosidase, lactase phlorizinhydrolase, |
| | | | | | | | and related proteins |
| 92 | GmaAffx.87812.2.S1_at | 2.898 | BI971676 | Signal Transduction | Protein Kinase | | |
| 93 | GmaAffx.14179.1.S1_at | 2.888 | BM085107 | Unclassified - Hypothetical | | | SETA binding protein SB 1 and related |
| | | | | Protein NOT Supported by | | | proteins, contain BTB/POZ domain |
| | | | | cDNA | | | |
| 94 | Gma.6288.1.S1_at | 2.888 | AW348556 | Transporter | | | Aquaporin (major intrinsic protein family) |
| 95 | GmaAffx.68088.1.S1_at | 2.883 | BG507880 | Signal Transduction | Protein Kinase | | |
| 96 | Gma.3988.1.S1_at | 2.879 | BE658819 | Unclassified - Hypothetical | | | |
| | | | | Protein Supported by cDNA | | | |
| 97 | GmaAffx.17811.1.S1_at | 2.879 | BI426517 | Transporter | | | Predicted transporter (major facilitator |
| | | | | | | | superfamily) |
| 98 | Gma.13027.1.A1_at | 2.877 | CD397394 | Transporter | | | Amino acid transporters |
| 99 | GmaAffx.10491.1.S1_at | 2.847 | AW598141 | No Homology to Known | | | |
| | | | | Proteins | | | |
| 100 | Gma.3777.1.S1_x_at | 2.847 | CD399954 | Unclassified - Hypothetical | | | |
| | | | | Protein Supported by cDNA | | | |
| 101 | Gma.9913.2.S1_a_at | 2.822 | CD390580 | Transcription | Transcription | WRKY | |
| | | | | | Factor | | |
| 102 | Gma.7970.1.S1_at | 2.816 | BI699189 | No Homology to Known | | | |
| | | | | Proteins | | | |
| 103 | GmaAffx.74573.1.S1_at | 2.809 | BU763133 | No Homology to Known | | | |
| | | | | Proteins | | | |
| 104 | GmaAffx.42583.1.A1_at | 2.8 | BG154898 | Transporter | Inorganic | Fe(I1) transporter | Fe2+/Zn2+ regulated transporter |
| | | | | | Solute | isolog family | |
| | | | | | Cotransporters | | |
| 105 | Gma.8459.1.S1_at | 2.791 | AF249317.1 | Signal Transduction | Protein Kinase | | Serine/threonine protein kinase |
| 106 | Gma.4832.1.A1_at | 2.765 | AW310458 | Signal Transduction | Protein Kinase | | |
| 107 | Gma.17559.1.S1_at | 2.764 | BG508388 | No Homology to Known | | | |
| | | | | Proteins | | | |
| 108 | Gma.17576.1.S1_s_at | 2.747 | BG650176 | No Homology to Known | | | |
| | | | | Proteins | | | |
| 109 | GmaAffx.60595.1.S1_at | 2.738 | BQ628072 | No Homology to Known | | | |
| | | | | Proteins | | | |
| 110 | GmaAffx.52050.1.S1_at | 2.707 | BG237350 | Transcription | Transcription | MYB | Transcription factor, Myb superfamily |
| | | | | | Factor | | |
| 111 | GmaAffx.85409.1.S1_at | 2.681 | BF598665 | Unclassified - Hypothetical | | | Copper chaperone |
| | | | | Protein NOT Supported by | | | |
| | | | | cDNA | | | |
| 112 | GmaAffx.88037.2.S1_at | 2.679 | CA783510 | Unclassified - Hypothetical | | | |
| | | | | Protein Supported by cDNA | | | |
| 113 | Gma.17605.3.S1_at | 2.678 | BQ079859 | Secondary Metabolism | Flavonoid | | |
| | | | | | Biosynthesis | | |
| 114 | GmaAffx.91346.1.S1_x_at | 2.634 | CF807071 | Unclassified - Hypothetical | | | |
| | | | | Protein Supported by cDNA | | | |
| 115 | GmaAffx.89999.1.S1_at | 2.613 | CF805724 | Disease & Defense | | | |
| 116 | Gma.17816.1.A1_at | 2.607 | BI970976 | Disease & Defense | | | |
| 117 | Gma.68.1.S1_at | 2.6 | AF357211.1 | Transcription | Transcription | AP2/EREBP | |
| | | | | | Factor | | |
| 118 | Gma.7921.1.S1_at | 2.59 | BM177567 | Unclassified - Hypothetical | | | Uncharacterized conserved protein |
| | | | | Protein Supported by cDNA | | | |
| 119 | GmaAffx.56917.1.S1_at | 2.564 | AI441130 | Signal Transduction | Protein Kinase | | Serine/threonine protein kinase |
| 120 | GmaAffx.28927.1.S1_at | 2.545 | BU549173 | Transcription | Transcription | MYB | Transcription factor, Myb superfamily |
| | | | | | Factor | | |
| 121 | GmaAffx.87812.1.S1_at | 2.543 | AW831390 | Signal Transduction | Protein Kinase | | |
| 122 | Gma.4169.1.S1_at | 2.53 | BQ473559 | Transporter | | | Copper chaperone |
| 123 | GmaAffx.91346.1.S1_at | 2.513 | CF807071 | Unclassified - Hypothetical | | | |
| | | | | Protein Supported by cDNA | | | |
| 124 | GmaAffx.4905.1.S1_at | 2.496 | BQ453808 | Metabolism | | | Predicted lipase |
| 125 | Gma.11257.1.S1_at | 2.477 | AW310476 | Cell Structure | | | |
| 126 | GmaAffx.16502.1.A1_at | 2.47 | BE659118 | No Homology to Known | | | |
| | | | | Proteins | | | |
| 127 | GmaAffx.90946.1.S1_at | 2.462 | CF808349 | Unclassified - Hypothetical | | | |
| | | | | Protein NOT Supported by | | | |
| | | | | cDNA | | | |
| 128 | GmaAffx.77809.1.A1_at | 2.447 | BI969302 | Disease & Defense | | | |
| 129 | GmaAffx.7215.1.A1_at | 2.443 | AW351149 | Unclassified - Hypothetical | | | |
| | | | | Protein NOT Supported by | | | |
| | | | | cDNA | | | |
| 130 | GmaAffx.13625.1.A1_at | 2.436 | BU547925 | No Homology to Known | | | |
| | | | | Proteins | | | |
| 131 | Gma.14471.4.S1_x_at | 2.434 | BQ081409 | Transporter | | | Aquaporin (major intrinsic protein family) |
| 132 | Gma.3136.1.S1_at | 2.429 | BG839241 | Metabolism | Cytochrome | CYP82C | Cytochrome P450 CYP2 subfamily |
| | | | | | P450 | | |
| 133 | GmaAffx.11658.1.S1_at | 2.427 | AW184817 | Signal Transduction | Unclassified | | |
| 134 | Gma.6529.2.S1_s_at | 2.426 | BM309390 | Unclassified - Hypothetical | | | |
| | | | | Protein NOT Supported by | | | |
| | | | | cDNA | | | |
| 135 | GmaAffx.45241.1.S1_at | 2.422 | BG157181 | Unclassified - Hypothetical | | | Glutaredoxin and related proteins |
| | | | | Protein Supported by cDNA | | | |
| 136 | Gma.4396.1.S1_at | 2.417 | BQ081294 | No Homology to Known | | | |
| | | | | Proteins | | | |
| 137 | Gma.742.1.A1_at | 2.395 | CD393482 | Unclassified - Protein with | | | |
| | | | | Unknown Function | | | |
| 138 | Gma.1521.1.S1_at | 2.394 | CD394504 | Cell Structure | | | Beta tubulin |
| 139 | GmaAffx.89629.1.S1_s_at | 2.387 | CK606095 | Unclassified - Protein with | | | |
| | | | | Unknown Function | | | |
| 140 | Gma.12465.1.S1_a_at | 2.384 | CD415130 | Unclassified - Hypothetical | | | |
| | | | | Protein Supported by cDNA | | | |
| 141 | Gma.11116.4.S1_s_at | 2.373 | CD408430 | Transporter | | | Aquaporin (major intrinsic protein family) |
| 142 | Gma.2352.1.S1_at | 2.369 | AI443511 | Unclassified - Protein with | | | FOG: Predicted E3 ubiquitin ligase |
| | | | | Unknown Function | | | |
| 143 | GmaAffx.81783.1.S1_at | 2.359 | AW101961 | Signal Transduction | Protein Kinase | | Serine/threonine protein kinase |
| 144 | Gma.6650.2.S1_at | 2.354 | BU762479 | Unclassified - Protein with | | | |
| | | | | Unknown Function | | | |
| 145 | GmaAffx.33259.1.S1_at | 2.351 | BQ785322 | Unclassified - Hypothetical | | | |
| | | | | Protein Supported by cDNA | | | |
| 146 | Gma.6650.1.S1_at | 2.315 | BQ629283 | Unclassified - Protein with | | | |
| | | | | Unknown Function | | | |
| 147 | GmaAffx.86059.1.S1_at | 2.302 | BF598759 | No Homology to Known | | | |
| | | | | Proteins | | | |
| 148 | GmaAffx.74289.1.S1_at | 2.295 | CA852521 | Transcription | Transcription | MYB | Transcription factor, Myb superfamily |
| | | | | | Factor | | |
| 149 | Gma.3149.1.S1_a_at | 2.287 | BU544575 | No Homology to Known | | | |
| | | | | Proteins | | | |
| 150 | Gma.9925.1.S1_at | 2.284 | AW307455 | No Homology to Known | | | |
| | | | | Proteins | | | |
| 151 | GmaAffx.85264.1.S1_at | 2.276 | BE058913 | Metabolism | | | |
| 152 | Gma.3189.2.S1_at | 2.261 | BG511492 | Signal Transduction | MAPKK | | Mitogen-activated protein kinase kinase |
| | | | | | | | (MAP2K) |
| 153 | Gma.14966.1.A1_at | 2.259 | CD416359 | Intracellular Traffic | | | Aquaporin (major intrinsic protein family) |
| 154 | Gma.8077.1.S1_at | 2.259 | BU547877 | Cell Structure | | | |
| 155 | Gma.43.1.A1_at | 2.256 | L12258.1 | Transporter | | | Aquaporin (major intrinsic protein family) |
| 156 | GmaAffx.30344.1.S1_at | 2.255 | AW349341 | Metabolism | | | Lipid phosphate phosphatase and related |
| | | | | | | | enzymes of the PAP2 family |
| 157 | Gma.666.1.S1_at | 2.247 | AW310734 | Unclassified - Protein with | Hormone | | Voltage-gated shaker-like K+ channel, subunit |
| | | | | Unknown Function | Induced | | beta/KCNAB |
| 158 | GmaAffx.70634.1.S1_at | 2.247 | BQ079384 | Protein Destination & Storage | | | FOG: Predicted E3 ubiquitin ligase |
| 159 | Gma.17347.1.S1_s_at | 2.238 | AW597676 | Transcription | Transcription | NAC Domain | |
| | | | | | Factor | | |
| 160 | Gma.9606.1.S1_at | 2.234 | AB086392.1 | Transporter | | | Sucrose transporter and related proteins |
| 161 | Gma.18088.1.S1_at | 2.232 | BI970254 | Unclassified - Protein with | | | |
| | | | | Unknown Function | | | |
| 162 | GmaAffx.91339.1.S1_at | 2.225 | CF807064 | Cell Structure | | | |
| 163 | Gma.5139.1.S1_at | 2.218 | AF243376.1 | Secondary Metabolism | | | Glutathione S-transferase |
| 164 | Gma.14846.1.A1_at | 2.218 | AW310268 | No Homology to Known | | | |
| | | | | Proteins | | | |
| 165 | Gma.5330.2.S1_at | 2.217 | BM092298 | Unclassified - Hypothetical | | | |
| | | | | Protein NOT Supported by | | | |
| | | | | cDNA | | | |
| 166 | GmaAffx.3697.1.S1_at | 2.212 | BE820647 | Disease & Defense | | | |
| 167 | GmaAffx.90252.1.S1_s_at | 2.201 | CF805977 | Unclassified - Hypothetical | | | |
| | | | | Protein Supported by cDNA | | | |
| 168 | GmaAffx.56272.1.S1_at | 2.2 | BU761188 | No Homology to Known | | | |
| | | | | Proteins | | | |
| 169 | Gma.3810.1.S1_s_at | 2.19 | BI972063 | Disease & Defense | | | |
| 170 | Gma.4368.1.S1_at | 2.185 | BQ080689 | Unclassified - Protein with | | | |
| | | | | Unknown Function | | | |
| 171 | GmaAffx.58555.1.S1_at | 2.184 | BQ298550 | Metabolism | Cytochrome | CYP82C | Cytochrome P450 CYP2 subfamily |
| | | | | | P450 | | |
| 172 | GmaAffx.73185.1.S1_at | 2.183 | BI701903 | No Homology to Known | | | |
| | | | | Proteins | | | |
| 173 | Gma.11076.1.S1_at | 2.178 | BE658385 | Unclassified - Hypothetical | | | |
| | | | | Protein Supported by cDNA | | | |
| 174 | GmaAffx.62710.1.S1_at | 2.167 | BE473384 | Unclassified - Hypothetical | | | |
| | | | | Protein NOT Supported by | | | |
| | | | | cDNA | | | |
| 175 | GmaAffx.82105.1.S1_at | 2.164 | AI938690 | Transporter | | | Sucrose transporter and related proteins |
| 176 | GmaAffx.50656.1.A1_at | 2.159 | BI970755 | No Homology to Known | | | |
| | | | | Proteins | | | |
| 177 | GmaAffx.31548.1.S1_at | 2.158 | BG044632 | Transcription | Transcription | Unclassified | Transcription factor, Myb superfamily |
| | | | | | Factor | | |
| 178 | GmaAffx.43291.1.S1_at | 2.148 | AI442332 | Metabolism | | | Lipid phosphate phosphatase and related |
| | | | | | | | enzymes of the PAP2 family |
| 179 | GmaAffx.93073.1.S1_s_at | 2.121 | CF808798 | Cell Structure | | | |
| 180 | GmaAffx.671.1.S1_at | 2.112 | CA785167 | Cell Structure | | | |
| 181 | GmaAffx.43620.1.S1_at | 2.089 | AW184956 | No Homology to Known | | | |
| | | | | Proteins | | | |
| 182 | Gma.17586.1.S1_at | 2.079 | BU546955 | Unclassified - Hypothetical | | | Reticulon |
| | | | | Protein Supported by cDNA | | | |
| 183 | GmaAffx.3363.1.S1_at | 2.074 | BE347006 | Unclassified - Hypothetical | | | |
| | | | | Protein NOT Supported by | | | |
| | | | | cDNA | | | |
| 184 | Gma.12465.2.S1_at | 2.073 | BE611098 | Unclassified - Hypothetical | | | |
| | | | | Protein Supported by cDNA | | | |
| 185 | GmaAffx.57341.1.S1_at | 2.067 | AW277978 | Transporter | | | Amino acid transporters |
| 186 | GmaAffx.77404.1.S1_at | 2.067 | CD393010 | Intracellular Traffic | | | GTPase Rab11/YPT3, small G protein |
| | | | | | | | superfamily |
| 187 | Gma.3576.2.S1_a_at | 2.061 | BI316058 | Unclassified - Protein with | | | Diadenosine polyphosphate hydrolase and |
| | | | | Unknown Function | | | related proteins of the histidine triad (HIT) |
| | | | | | | | family |
| 188 | Gma.612.1.A1_at | 2.06 | AI442942 | Transcription | Transcription | AP2/EREBP | |
| | | | | | Factor | | |
| 189 | Gma.3555.1.S1_at | 2.059 | BU764259 | Unclassified - Hypothetical | | | |
| | | | | Protein NOT Supported by | | | |
| | | | | cDNA | | | |
| 190 | GmaAffx.8013.1.S1_at | 2.053 | BE609266 | Unclassified - Hypothetical | | | |
| | | | | Protein NOT Supported by | | | |
| | | | | cDNA | | | |
| 191 | Gma.10441.1.S1_s_at | 2.031 | BE803701 | Unclassified - Protein with | | | |
| | | | | Unknown Function | | | |
| 192 | GmaAffx.79401.1.S1_at | 2.027 | CA936959 | Transcription | Transcription | bZIP | |
| | | | | | Factor | | |
| 193 | GmaAffx.18518.1.S1_at | 2.016 | BI700329 | Cell Structure | | | |
| 194 | GmaAffx.46974.1.S1_at | 2.014 | BU081847 | Signal Transduction | Protein Kinase | | |
| 195 | GmaAffx.23953.1.S1_s_at | 2.005 | BE658262 | Transporter | | | |
| 196 | Gma.13973.1.A1_at | 2.004 | CD412168 | No Homology to Known | | | |
| | | | | Proteins | | | |
| 197 | Gma.6375.1.S1_at | 0.498 | BQ610190 | Unclassified - Hypothetical | | | AAA+-type ATPase |
| | | | | Protein NOT Supported by | | | |
| | | | | cDNA | | | |
| 198 | Gma.1528.1.S1_at | 0.498 | CD403623 | Transcription | Transcription | Basic Helix- | |
| | | | | | Factor | Loop-Helix | |
| | | | | | | (bHLH) | |
| 199 | GmaAffx.85469.1.S1_at | 0.497 | BI786397 | Unclassified - Hypothetical | | | |
| | | | | Protein NOT Supported by | | | |
| | | | | cDNA | | | |
| 200 | Gma.1642.3.S1_s_at | 0.493 | BM891895 | Unclassified - Hypothetical | | | |
| | | | | Protein Supported by cDNA | | | |
| 201 | Gma.7371.2.S1_a_at | 0.493 | BQ741076 | Cell Structure | | | Dynein light chain type 1 |
| 202 | GmaAffx.7302.2.A1_s_at | 0.488 | BE820704 | No Homology to Known | | | |
| | | | | Proteins | | | |
| 203 | Gma.13039.1.A1_at | 0.488 | CD399750 | Unclassified - Hypothetical | | | |
| | | | | Protein NOT Supported by | | | |
| | | | | cDNA | | | |
| 204 | GmaAffx.81502.2.S1_at | 0.486 | BG041291 | Unclassified - Hypothetical | | | Uncharacterized conserved protein |
| | | | | Protein NOT Supported by | | | |
| | | | | cDNA | | | |
| 205 | GmaAffx.63115.1.A1_at | 0.485 | BE823736 | Unclassified - Hypothetical | | | |
| | | | | Protein NOT Supported by | | | |
| | | | | cDNA | | | |
| 206 | GmaAffx.53069.2.S1_at | 0.483 | AW309012 | No Homology to Known | | | |
| | | | | Proteins | | | |
| 207 | GmaAffx.90242.1.S1_s_at | 0.483 | CF805967 | No Homology to Known | | | |
| | | | | Proteins | | | |
| 208 | GmaAffx.73857.1.S1_at | 0.48 | BM885538 | Unclassified - Hypothetical | | | |
| | | | | Protein Supported by cDNA | | | |
| 209 | GmaAffx.50635.2.S1_at | 0.48 | AW733509 | Metabolism | Cytochrome | CYP707A | Cytochrome P450 CYP4/CYP19/CYP26 |
| | | | | | P450 | | subfamilies |
| 210 | GmaAffx.86667.1.S1_at | 0.48 | CA938472 | Unclassified - Hypothetical | | | |
| | | | | Protein Supported by cDNA | | | |
| 211 | GmaAffx.71032.1.S1_at | 0.477 | AW278168 | Unclassified - Hypothetical | | | |
| | | | | Protein NOT Supported by | | | |
| | | | | cDNA | | | |
| 212 | GmaAffx.76295.1.S1_at | 0.476 | BE347326 | Unclassified - Protein with | Hormone | | |
| | | | | Unknown Function | Induced | | |
| 213 | GmaAffx.30593.1.S1_at | 0.474 | BE803365 | Unclassified - Hypothetical | | | |
| | | | | Protein Supported by cDNA | | | |
| 214 | GmaAffx.76699.1.S1_at | 0.473 | AW760679 | Transcription | Transcription | Basic Helix- | |
| | | | | | Factor | Loop-Helix | |
| | | | | | | (bHLH) | |
| 215 | GmaAffx.78358.2.S1_at | 0.472 | BU762686 | Metabolism | | | |
| 216 | GmaAffx.79730.1.S1_at | 0.471 | BF325482 | Transcription | Transcription | Basic Helix- | |
| | | | | | Factor | Loop-Helix | |
| | | | | | | (bHLH) | |
| 217 | GmaAffx.23475.2.S1_s_at | 0.467 | AW620563 | Unclassified - Hypothetical | | | |
| | | | | Protein Supported by cDNA | | | |
| 218 | Gma.7256.1.S1_at | 0.466 | L01447.1 | Transcription | Transcription | Basic Leucine | |
| | | | | | Factor | Zipper (bZIP) | |
| 219 | Gma.13103.1.S1_at | 0.466 | CD392133 | Unclassified - Hypothetical | | | |
| | | | | Protein NOT Supported by | | | |
| | | | | cDNA | | | |
| 220 | Gma.7040.1.A1_at | 0.462 | BE346424 | Transcription | Transcription | AP2/EREBP | |
| | | | | | Factor | | |
| 221 | GmaAffx.27488.1.S1_at | 0.459 | AW311080 | Signal Transduction | MAPKKK | Raf | Tyrosine kinase specific for activated (GTP- |
| | | | | | | | bound) p21cdc42Hs |
| 222 | GmaAffx.6017.1.A1_at | 0.458 | BU550467 | Unclassified - Hypothetical | | | |
| | | | | Protein NOT Supported by | | | |
| | | | | cDNA | | | |
| 223 | GmaAffx.90899.1.S1_at | 0.447 | CF806624 | Unclassified - Hypothetical | | | |
| | | | | Protein NOT Supported by | | | |
| | | | | cDNA | | | |
| 224 | Gma.1951.2.S1_at | 0.445 | CA783485 | Protein Destination & Storage | | | Serine carboxypeptidases |
| 225 | Gma.10591.2.S1_at | 0.444 | CD414199 | Metabolism | | | Glycine cleavage system H protein (lipoate- |
| | | | | | | | binding) |
| 226 | GmaAffx.25912.1.S1_at | 0.443 | BU545028 | Unclassified - Protein with | | | |
| | | | | Unknown Function | | | |
| 227 | GmaAffx.69307.1.S1_at | 0.442 | BQ473855 | No Homology to Known | | | |
| | | | | Proteins | | | |
| 228 | Gma.2066.1.S1_a_at | 0.44 | BG363704 | No Homology to Known | | | |
| | | | | Proteins | | | |
| 229 | Gma.11194.2.S1_at | 0.438 | BM886635 | Energy | | | Thioredoxin |
| 230 | Gma.10100.1.S1_at | 0.438 | BE023275 | No Homology to Known | | | |
| | | | | Proteins | | | |
| 231 | Gma.2827.1.S1_at | 0.433 | BG237560 | Unclassified - Hypothetical | | | FOG: PPR repeat |
| | | | | Protein Supported by cDNA | | | |
| 232 | GmaAffx.56319.1.S1_at | 0.433 | BE802287 | Metabolism | | | Uncharacterized conserved protein |
| 233 | GmaAffx.26667.1.S1_at | 0.432 | BE806932 | No Homology to Known | | | |
| | | | | Proteins | | | |
| 234 | GmaAffx.87900.1.S1_at | 0.429 | BI892943 | Protein Destination & Storage | | | FKBP-type peptidyl-prolyl cis-trans isomerase |
| 235 | GmaAffx.90113.1.S1_at | 0.428 | CF806004 | Protein Destination & Storage | Storage Protein | | Uncharacterized conserved protein |
| 236 | Gma.10393.1.S1_at | 0.422 | BE804608 | Unclassified - Hypothetical | | | Endoplasmic reticulum protein EP58, contains |
| | | | | Protein NOT Supported by | | | filamin rod domain and KDEL motif |
| | | | | cDNA | | | |
| 237 | GmaAffx.948.1.S1_at | 0.421 | BG155389 | Protein Destination & Storage | | | Ubiquitin fusion-degradation protein |
| 238 | PsAffx.psHB026xH01f_s_at | 0.419 | | | | | |
| 239 | Gma.8501.1.S1_at | 0.418 | AF184278.1 | Transcription | Transcription | Homeodomain | Transcription factor HEX, contains HOX and |
| | | | | | Factor | | HALZ domains |
| 240 | GmaAffx.83226.1.S1_at | 0.417 | CA852250 | Transcription | Transcription | Unclassified | |
| | | | | | Factor | | |
| 241 | GmaAffx.84566.3.S1_s_at | 0.411 | BG045916 | Transcription | Transcription | MYB | Transcription factor, Myb superfamily |
| | | | | | Factor | | |
| 242 | GmaAffx.25068.2.A1_at | 0.409 | CD413732 | Metabolism | Amino Acid | | |
| | | | | | Biosynthesis/ | | |
| | | | | | Metabolism | | |
| 243 | Gma.13019.1.A1_s_at | 0.402 | CD392967 | Secondary Metabolism | | | Hydroxyindole-O-methyltransferase and |
| | | | | | | | related SAM-dependent methyltransferases |
| 244 | GmaAffx.11496.1.S1_at | 0.4 | BE821691 | Transporter | Inorganic | Putative sulfate | Sulfate/bicarbonate/oxalate exchanger SAT-1 |
| | | | | | Solute | transporter | and related transporters (SLC26 family) |
| | | | | | Cotransporters | family | |
| 245 | GmaAffx.494.1.S1_at | 0.4 | AI938330 | Cell Structure | | | Chitinase |
| 246 | GmaAffx.52526.1.S1_at | 0.399 | BM085509 | Unclassified - Hypothetical | | | Uncharacterized conserved protein |
| | | | | Protein Supported by cDNA | | | |
| 247 | Gma.17402.1.A1_at | 0.398 | BE611084 | No Homology to Known | | | |
| | | | | Proteins | | | |
| 248 | GmaAffx.37316.1.S1_at | 0.398 | AW733720 | Unclassified - Protein with | | | |
| | | | | Unknown Function | | | |
| 249 | Gma.137.1.S1_at | 0.397 | AF022464.1 | Metabolism | Cytochrome | | Cytochrome P450 CYP2 subfamily |
| | | | | | P450 | | |
| 250 | Gma.17594.2.S1_at | 0.397 | BM093196 | Metabolism | | | Cystathionine beta-lyases/cystathionine gamma |
| | | | | | | | synthases |
| 251 | Gma.10873.1.S1_s_at | 0.395 | BQ079465 | No Homology to Known | | | |
| | | | | Proteins | | | |
| 252 | GmaAffx.3369.1.S1_at | 0.395 | BU548983 | Cell Structure | | | |
| 253 | GmaAffx.86212.1.S1_at | 0.394 | BG043474 | Unclassified - Hypothetical | | | |
| | | | | Protein NOT Supported by | | | |
| | | | | cDNA | | | |
| 254 | GmaAffx.62153.1.S1_at | 0.394 | AW394575 | No Homology to Known | | | |
| | | | | Proteins | | | |
| 255 | GmaAffx.37988.1.S1_at | 0.392 | BF324942 | No Homology to Known | | | |
| | | | | Proteins | | | |
| 256 | GmaAffx.72807.1.S1_at | 0.391 | AW831487 | No Homology to Known | | | |
| | | | | Proteins | | | |
| 257 | GmaAffx.70659.1.S1_at | 0.39 | AI495240 | No Homology to Known | | | |
| | | | | Proteins | | | |
| 258 | GmaAffx.91358.1.S1_x_at | 0.39 | CF807083 | No Homology to Known | | | |
| | | | | Proteins | | | |
| 259 | GmaAffx.80584.1.S1_at | 0.388 | BU083387 | Transcription | Transcription | Basic Helix- | |
| | | | | | Factor | Loop-Helix | |
| | | | | | | (bHLH) | |
| 260 | GmaAffx.70548.1.A1_at | 0.388 | CD411723 | No Homology to Known | | | |
| | | | | Proteins | | | |
| 261 | GmaAffx.71038.1.S1_at | 0.386 | BI700350 | Transcription | Transcription | Homeodomain | Transcription factor MEIS1 and related HOX |
| | | | | | Factor | | domain proteins |
| 262 | Gma.5701.3.S1_at | 0.384 | CA852693 | No Homology to Known | | | |
| | | | | Proteins | | | |
| 263 | GmaAffx.15792.1.A1_at | 0.384 | AW101026 | No Homology to Known | | | |
| | | | | Proteins | | | |
| 264 | Gma.3204.1.S1_at | 0.383 | AW509002 | Metabolism | | | Dehydrogenase kinase |
| 265 | Gma.4506.1.S1_at | 0.382 | BQ740943 | Unclassified - Hypothetical | | | |
| | | | | Protein NOT Supported by | | | |
| | | | | cDNA | | | |
| 266 | Gma.3384.1.S1_at | 0.378 | BE659400 | Unclassified - Protein with | | | Acyl-CoA reductase |
| | | | | Unknown Function | | | |
| 267 | GmaAffx.7838.3.S1_s_at | 0.378 | CA800410 | Metabolism | | | |
| 268 | GmaAffx.40114.1.A1_at | 0.378 | BE661587 | Unclassified - Hypothetical | | | |
| | | | | Protein Supported by cDNA | | | |
| 269 | GmaAffx.85657.1.S1_s_at | 0.378 | AW185911 | Unclassified - Protein with | | | FOG: Kelch repeat |
| | | | | Unknown Function | | | |
| 270 | GmaAffx.90812.1.S1_at | 0.373 | CF806537 | Disease & Defense | | | |
| 271 | Gma.16910.1.S1_at | 0.367 | BU549219 | Unclassified - Protein with | | | |
| | | | | Unknown Function | | | |
| 272 | GmaAffx.81999.1.S1_at | 0.36 | AI416637 | Transcription | Transcription | Homeodomain | Transcription factor ME1S1 and related HOX |
| | | | | | Factor | | domain proteins |
| 273 | Gma.5725.1.S1_at | 0.36 | BM886367 | Disease & Defense | | | |
| 274 | Gma.3901.1.S1_at | 0.358 | CD394788 | Protein Destination & Storage | Storage Protein | | |
| 275 | Gma.13845.1.A1_at | 0.355 | CD407676 | Unclassified - Hypothetical | | | |
| | | | | Protein Supported by cDNA | | | |
| 276 | GmaAffx.1620.2.S1_at | 0.355 | BF324548 | Unclassified - Hypothetical | | | |
| | | | | Protein NOT Supported by | | | |
| | | | | cDNA | | | |
| 277 | Gma.790.1.A1_s_at | 0.351 | BE820450 | Unclassified - Hypothetical | | | Predicted E3 ubiquitin ligase |
| | | | | Protein Supported by cDNA | | | |
| 278 | GmaAffx.15471.3.S1_x_at | 0.351 | BE346777 | Transcription | Transcription | MYB | Transcription factor, Myb superfamily |
| | | | | | Factor | | |
| 279 | GmaAffx.75459.1.S1_s_at | 0.348 | BE348034 | Protein Destination & Storage | Storage Protein | | |
| 280 | GmaAffx.58606.1.S1_at | 0.341 | BM526502 | Metabolism | | | |
| 281 | Gma.10892.1.S1_a_at | 0.341 | BG839570 | Metabolism | | | Predicted carbonic anhydrase involved in |
| | | | | | | | protection against oxidative damage |
| 282 | GmaAffx.78768.2.S1_at | 0.335 | AI930773 | Unclassified - Hypothetical | | | |
| | | | | Protein Supported by cDNA | | | |
| 283 | Gma.221.1.S1_at | 0.328 | U41475.1 | Metabolism | | | Phosphoinositide-specific phospholipase C |
| 284 | Gma.12987.1.S1_at | 0.327 | CD392747 | Transcription | Transcription | Basic Helix- | |
| | | | | | Factor | Loop-Helix | |
| | | | | | | (bHLH) | |
| 285 | GmaAffx.50980.1.S1_s_at | 0.327 | BE823095 | Signal Transduction | Protein | | |
| | | | | | Phosphatase | | |
| 286 | GmaAffx.32011.2.S1_at | 0.322 | BM084966 | Transcription | Transcription | Basic Leucine | |
| | | | | | Factor | Zipper (bZIP) | |
| 287 | GmaAffx.80188.1.A1_at | 0.317 | AW349832 | No Homology to Known | | | |
| | | | | Proteins | | | |
| 288 | Gma.13030.1.S1_a_at | 0.316 | AI748012 | Protein Destination & Storage | | | |
| 289 | Gma.9365.1.A1_at | 0.309 | CD418433 | No Homology to Known | | | |
| | | | | Proteins | | | |
| 290 | Gma.13273.1.A1_at | 0.306 | CD397107 | Unclassified - Hypothetical | | | |
| | | | | Protein Supported by cDNA | | | |
| 291 | GmaAffx.64807.1.S1_at | 0.301 | AW201972 | No Homology to Known | | | |
| | | | | Proteins | | | |
| 292 | Gma.1773.1.A1_at | 0.298 | CD404050 | Transcription | Transcription | Basic Helix- | |
| | | | | | Factor | Loop-Helix | |
| | | | | | | (bHLH) | |
| 293 | GmaAffx.89697.1.S1_s_at | 0.296 | CK606333 | Disease & Defense | | | |
| 294 | Gma.3617.1.S1_at | 0.282 | BE659297 | Cell Growth & Division | | | |
| 295 | GmaAffx.74438.1.S1_at | 0.282 | BE440325 | Unclassified - Hypothetical | | | |
| | | | | Protein NOT Supported by | | | |
| | | | | cDNA | | | |
| 296 | Gma.8058.1.A1_at | 0.276 | BM271479 | Signal Transduction | Protein | | Serine/threonine protein phosphatase |
| | | | | | Phosphatase | | |
| 297 | Gma.1768.1.S1_s_at | 0.276 | BE820777 | Unclassified - Hypothetical | | | |
| | | | | Protein Supported by cDNA | | | |
| 298 | GmaAffx.40114.1.S1_at | 0.267 | BI973325 | Unclassified - Hypothetical | | | |
| | | | | Protein Supported by cDNA | | | |
| 299 | GmaAffx.52826.3.S1_at | 0.252 | BF596503 | Signal Transduction | Protein | | Serine/threonine protein phosphatase |
| | | | | | Phosphatase | | |
| 300 | GmaAffx.57432.1.S1_at | 0.25 | BE657443 | Unclassified - Hypothetical | | | |
| | | | | Protein NOT Supported by | | | |
| | | | | cDNA | | | |
| 301 | Gma.13672.1.A1_at | 0.242 | CD403249 | No Homology to Known | | | |
| | | | | Proteins | | | |
| 302 | Gma.8674.3.A1_at | 0.238 | BI469914 | Transporter | | | Predicted transporter (major facilitator |
| | | | | | | | superfamily) |
| 303 | Gma.6684.1.S1_at | 0.236 | AW459859 | Signal Transduction | Unclassified | | Lanthionine synthetase C-like protein 1 |
| 304 | Gma.16500.1.S1_at | 0.235 | BE059054 | Metabolism | | | |
| 305 | GmaAffx.58712.1.S1_at | 0.233 | BF325034 | Transporter | | | Transporter, ABC superfamily (Breast cancer |
| | | | | | | | resistance protein) |
| 306 | Gma.7006.1.S1_at | 0.223 | AF516880.1 | Cell Structure | | | |
| 307 | Gma.4252.1.S1_at | 0.216 | BE821208 | Metabolism | | | Glutamate decarboxylase and related proteins |
| 308 | GmaAffx.66215.1.S1_at | 0.215 | AI443109 | Signal Transduction | Unclassified | | Lanthionine synthetase C-like protein 1 |
| 309 | GmaAffx.15862.1.S1_at | 0.211 | BM092634 | Cell Structure | | | |
| 310 | GmaAffx.79283.1.S1_at | 0.208 | CA819076 | Unclassified - Hypothetical | | | |
| | | | | Protein NOT Supported by | | | |
| | | | | cDNA | | | |
| 311 | GmaAffx.92964.1.S1_at | 0.206 | CF808689 | No Homology to Known | | | |
| | | | | Proteins | | | |
| 312 | Gma.6333.2.S1_at | 0.202 | CD396361 | No Homology to Known | | | |
| | | | | Proteins | | | |
| 313 | Gma.1426.1.A1_at | 0.202 | AI443364 | Metabolism | | | |
| 314 | Gma.12068.1.A1_at | 0.192 | BU547739 | Transporter | | | Transporter, ABC superfamily (Breast cancer |
| | | | | | | | resistance protein) |
| 315 | Gma.6921.1.S1_at | 0.188 | BE823209 | Signal Transduction | Unclassified | | Phosphatidylethanolamine binding protein |
| 316 | GmaAffx.93220.1.S1_at | 0.18 | CF808945 | No Homology to Known | | | |
| | | | | Proteins | | | |
| 317 | Gma.2382.1.S1_x_at | 0.171 | BG789835 | Metabolism | | | |
| 318 | GmaAffx.92167.1.A1_s_at | 0.164 | CF807892 | Unclassified - Hypothetical | | | |
| | | | | Protein NOT Supported by | | | |
| | | | | cDNA | | | |
| 319 | Gma.857.1.S1_at | 0.143 | BE822264 | No Homology to Known | | | |
| | | | | Proteins | | | |
| 320 | GmaAffx.57432.2.S1_at | 0.13 | CA819434 | No Homology to Known | | | |
| | | | | Proteins | | | |
| 321 | Gma.10999.1.S1_at | 0.124 | CA850772 | Disease & Defense | | | |
| 322 | GmaAffx.88546.1.S1_at | 0.114 | BE801134 | Metabolism | | | |
| 323 | Gma.11993.1.S1_at | 0.107 | BU551348 | Transporter | | | Transporter, ABC superfamily (Breast cancer |
| | | | | | | | resistance protein) |
| 324 | Gma.2382.1.S1_s_at | 0.107 | BG789835 | Metabolism | | | |
| 325 | GmaAffx.72198.1.A1_at | 0.099 | BE823481 | Metabolism | | | UDP-glucuronosyl and UDP-glucosyl |
| | | | | | | | transferase |
| 326 | GmaAffx.93212.1.S1_s_at | 0.0863 | CF807524 | Transporter | | | |
| 327 | Gma.7831.1.S1_s_at | 0.0815 | CD393602 | Transporter | | | |
| 328 | Gma.1061.1.A1_at | 0.0594 | CD408183 | Metabolism | | | |
| 329 | GmaAffx.93212.1.S1_at | 0.0532 | CF807524 | Transporter | | | |
| 330 | Gma.5929.1.S1_at | 0.0517 | BQ610446 | No Homology to Known | | | |
| | | | | Proteins | | | |
| 331 | GmaAffx.63464.1.S1_at | 0.0409 | BG156739 | No Homology to Known | | | |
| | | | | Proteins | | | |
| 332 | Gma.10904.1.S1_at | 0.0244 | CD408184 | Protein Destination & Storage | | | |
| 333 | Gma.10904.1.S1_s_at | 0.0156 | CD408184 | Protein Destination & Storage | | | |

TABLE 13

| | | Fold | | | | | |
|---|---|---|---|---|---|---|---|
| ID | Gene Name | Change | Soybean_GenBank_Acc | Functional Category | Sub-Family | Gene Family | Additional Description |
| 1 | Gma.4058.1.S1_at | 5.006 | AW310338 | Transporter | | | Predicted transporter (major facilitator |
| | | | | | | | superfamily) |
| 2 | Gma.8301.2.S1_at | 3.484 | BU544524 | Signal Transduction | Calcium | | |
| | | | | | Binding | | |
| 3 | Gma.8416.1.S1_at | 3.472 | U13987.1 | Metabolism | | | Cytochrome b5 |
| 3 | Gma.8416.1.S1_at | 3.472 | U13987.1 | Metabolism | | | NADH-cytochrome b-5 reductase |
| 3 | Gma.8416.1.S1_at | 3.472 | U13987.1 | Metabolism | | | Sulfite oxidase, molybdopterin-binding |
| | | | | | | | component |
| 4 | GmaAffx.66589.1.S1_at | 3.45 | BE821501 | Transcription | Transcription | Polycomb Group | |
| | | | | | Factor | | |
| 5 | Gma.1221.1.S1_s_at | 3.365 | L23854.1 | Metabolism | | | Cytochrome b5 |
| 5 | Gma.1221.1.S1_s_at | 3.365 | L23854.1 | Metabolism | | | NADH-cytochrome b-5 reductase |
| 5 | Gma.1221.1.S1_s_at | 3.365 | L23854.1 | Metabolism | | | Sulfite oxidase, molybdopterin-binding |
| | | | | | | | component |
| 6 | Gma.10921.1.S1_s_at | 3.348 | BQ611250 | No Homology to Known | | | |
| | | | | Proteins | | | |
| 7 | GmaAffx.62542.1.S1_at | 3.33 | BE659136 | Unclassified - Hypothetical | | | |
| | | | | Protein NOT Supported by | | | |
| | | | | cDNA | | | |
| 8 | GmaAffx.67477.2.S1_at | 3.177 | AW509184 | Cell Structure | | | |
| 9 | GmaAffx.19116.1.S1_at | 3.012 | B1968419 | Signal Transduction | Protein Kinase | | |
| 10 | Gma.10307.1.S1_at | 2.93 | CA936845 | Unclassified - Hypothetical | | | |
| | | | | Protein NOT Supported by | | | |
| | | | | cDNA | | | |
| 11 | Gma.12356.1.A1_at | 2.882 | CD397798 | Unclassified - Hypothetical | | | |
| | | | | Protein NOT Supported by | | | |
| | | | | cDNA | | | |
| 12 | GmaAffx.13179.1.S1_at | 2.769 | CD402274 | Unclassified - Hypothetical | | | |
| | | | | Protein NOT Supported by | | | |
| | | | | cDNA | | | |
| 13 | Gma.6266.1.A1_at | 2.64 | CD413418 | Unclassified - Protein with | | | |
| | | | | Unknown Function | | | |
| 14 | Gma.5689.3.S1_s_at | 2.631 | BE657645 | Protein Destination & Storage | | | Gelatinase A and related matrix |
| | | | | | | | metalloproteases |
| 15 | Gma.2194.1.S1_at | 2.524 | BE657352 | Unclassified - Hypothetical | | | |
| | | | | Protein Supported by cDNA | | | |
| 16 | Gma.17789.1.S1_at | 2.408 | BG511704 | Unclassified - Hypothetical | | | |
| | | | | Protein Supported by cDNA | | | |
| 17 | GmaAffx.90042.1.S1_at | 2.372 | CF805767 | Signal Transduction | Calcium | | Calmodulin and related proteins (EF- |
| | | | | | Binding | | Hand superfamily) |
| 18 | GmaAffx.30420.1.S1_at | 2.207 | AW704927 | Transporter | | | |
| 19 | Gma.14802.1.S1_at | 2.141 | AW201116 | Unclassified - Protein with | | | FOG: Ankyrin repeat |
| | | | | Unknown Function | | | |
| 20 | Gma.964.1.A1_at | 2.128 | BE824146 | Metabolism | | | Tyrosine aminotransferase |
| 21 | Gma.869.1.S1_at | 2.125 | BE822531 | Protein Destination & Storage | | | Serine carboxypeptidases (lysosomal |
| | | | | | | | cathepsin A) |
| 22 | GmaAffx.93462.1.S1_s_at | 2.116 | CF809187 | Signal Transduction | Protein Kinase | AtC1PKs | Serine/threonine protein kinase |
| 23 | GmaAffx.31180.1.A1_at | 2.113 | BE820254 | No Homology to Known | | | |
| | | | | Proteins | | | |
| 24 | GmaAffx.56804.1.A1_at | 2.111 | CD415337 | Unclassified - Hypothetical | | | Uncharacterized conserved protein, |
| | | | | Protein NOT Supported by | | | contains TraB domain |
| | | | | cDNA | | | |
| 25 | GmaAffx.83103.2.S1_at | 2.072 | BE806704 | Unclassified - Hypothetical | | | |
| | | | | Protein NOT Supported by | | | |
| | | | | cDNA | | | |
| 26 | GmaAffx.87806.1.S1_at | 2.032 | BG882829 | Unclassified - Hypothetical | | | |
| | | | | Protein NOT Supported by | | | |
| | | | | cDNA | | | |
| 27 | Gma.4701.2.A1_a_at | 2.011 | BM526048 | Energy | | | Thioredoxin |
| 28 | Gma.10787.2.S1_a_at | 0.5 | BE023083 | Transcription | Transcription | Squamosa | |
| | | | | | Factor | | |
| 29 | GmaAffx.84721.1.S1_at | 0.5 | BQ297863 | Metabolism | | | Hydroxymethylglutaryl-CoA lyase |
| 30 | GmaAffx.90970.1.S1_s_at | 0.5 | CF806695 | Unclassified - Hypothetical | | | |
| | | | | Protein Supported by cDNA | | | |
| 31 | Gma.12821.1.S1_s_at | 0.499 | CD391504 | No Homology to Known | | | |
| | | | | Proteins | | | |
| 32 | GmaAffx.71386.1.S1_at | 0.499 | AW704747 | Protein Destination & Storage | | | Molecular chaperone (DnaJ superfamily) |
| 33 | GmaAffx.23289.1.S1_at | 0.499 | CD392087 | No Homology to Known | | | |
| | | | | Proteins | | | |
| 34 | Gma.8782.1.A1_at | 0.499 | AW309953 | Disease & Defense | | | Apoptotic ATPase |
| 35 | Gma.17191.1.S1_at | 0.499 | BG237724 | Metabolism | | | UDP-glucuronosyl and UDP-glucosyl |
| | | | | | | | transferase |
| 36 | GmaAffx.72346.1.S1_at | 0.499 | BF424359 | Unclassified - Hypothetical | | | Speckle-type POZ protein SPOP and |
| | | | | Protein NOT Supported by | | | related proteins with TRAF, MATH and |
| | | | | cDNA | | | BTB/POZ domains |
| 37 | GmaAffx.77527.1.S1_at | 0.499 | BF423510 | Unclassified - Hypothetical | | | GTPase Rab18, small G protein |
| | | | | Protein Supported by cDNA | | | superfamily |
| 38 | GmaAffx.87922.1.S1_at | 0.499 | BU084547 | Energy | | | Pyrophosphate-dependent |
| | | | | | | | phosphofructo-1-kinase |
| 39 | GmaAffx.65393.1.S1_s_at | 0.499 | BU760770 | No Homology to Known | | | |
| | | | | Proteins | | | |
| 40 | GmaAffx.75708.1.S1_at | 0.497 | BF599014 | Unclassified - Hypothetical | | | |
| | | | | Protein Supported by cDNA | | | |
| 41 | Gma.6025.1.S1_at | 0.497 | CD399734 | Unclassified - Hypothetical | | | |
| | | | | Protein NOT Supported by | | | |
| | | | | cDNA | | | |
| 42 | GmaAffx.36972.2.S1_at | 0.496 | B1699120 | Unclassified - Hypothetical | | | |
| | | | | Protein NOT Supported by | | | |
| | | | | cDNA | | | |
| 43 | Gma.6528.2.S1_at | 0.496 | CA784209 | Transporter | | | Calcium transporting ATPase |
| 44 | Gma.7835.1.S1_at | 0.496 | CD410897 | Cell Growth & Division | | | |
| 45 | GmaAffx.6565.1.A1_at | 0.496 | CD402930 | No Homology to Known | | | |
| | | | | Proteins | | | |
| 46 | GmaAffx.71778.1.S1_at | 0.495 | BE346999 | Unclassified - Hypothetical | | | |
| | | | | Protein NOT Supported by | | | |
| | | | | cDNA | | | |
| 47 | GmaAffx.1128.1.S1_at | 0.495 | AW348608 | Unclassified - Protein with | | | Copper chaperone |
| | | | | Unknown Function | | | |
| 48 | GmaAffx.53585.2.S1_at | 0.495 | BE556399 | Protein Destination & Storage | | | Molecular chaperone (DnaJ superfamily) |
| 49 | GmaAffx.37484.2.S1_at | 0.494 | BQ629925 | Unclassified - Protein with | | | Predicted membrane protein, contains |
| | | | | Unknown Function | | | DoH and Cytochrome b-561/ferric |
| | | | | | | | reductase transmembrane domains |
| 50 | Gma.14155.3.S1_at | 0.494 | AI437500 | Unclassified - Hypothetical | | | Clathrin assembly protein AP180 and |
| | | | | Protein Supported by cDNA | | | related proteins, contain ENTH domain |
| 51 | Gma.1677.2.A1_at | 0.494 | AW348386 | Secondary Metabolism | | | |
| 52 | GmaAffx.50632.1.A1_at | 0.494 | B1969115 | Metabolism | | | Malate dehydrogenase |
| 53 | Gma.6722.1.S1_at | 0.494 | BE822185 | Unclassified - Hypothetical | | | UDP-glucuronosyl and UDP-glucosyl |
| | | | | Protein NOT Supported by | | | transferase |
| | | | | cDNA | | | |
| 54 | GmaAffx.62195.1.S1_at | 0.493 | AW472439 | No Homology to Known | | | |
| | | | | Proteins | | | |
| 55 | GmaAffx.11383.1.S1_at | 0.493 | BE822014 | Unclassified - Hypothetical | | | Kinesin light chain |
| | | | | Protein NOT Supported by | | | |
| | | | | cDNA | | | |
| 56 | GmaAffx.77651.1.S1_at | 0.493 | BF010272 | Unclassified - Protein with | Hormone | | Zinc-binding oxidoreductase |
| | | | | Unknown Function | Induced | | |
| 57 | Gma.9717.1.S1_at | 0.492 | BG653084 | Unclassified - Hypothetical | | | Amino acid transporters |
| | | | | Protein NOT Supported by | | | |
| | | | | cDNA | | | |
| 58 | GmaAffx.21553.1.S1_at | 0.492 | BE023327 | Protein Destination & Storage | | | Gelatinase A and related matrix |
| | | | | | | | metalloproteases |
| 59 | GmaAffx.90952.1.S1_at | 0.491 | CF806677 | Signal Transduction | Unclassified | | |
| 60 | Gma.3175.1.S1_at | 0.491 | A1736154 | Unclassified - Hypothetical | | | |
| | | | | Protein NOT Supported by | | | |
| | | | | cDNA | | | |
| 61 | GmaAffx.84871.1.S1_at | 0.491 | BF599103 | Signal Transduction | Protein Kinase | | |
| 62 | GmaAffx.56020.1.S1_at | 0.49 | BU080527 | Unclassified - Hypothetical | | | |
| | | | | Protein NOT Supported by | | | |
| | | | | cDNA | | | |
| 63 | GmaAffx.765.2.S1_at | 0.49 | AW509310 | Transporter | | | Amino acid transporters |
| 64 | GmaAffx.86283.1.S1_at | 0.49 | B1892496 | Unclassified - Hypothetical | | | Predicted pyroglutamyl peptidase |
| | | | | Protein Supported by cDNA | | | |
| 65 | Gma.9436.1.S1_at | 0.49 | CA802734 | Unclassified - Hypothetical | | | |
| | | | | Protein NOT Supported by | | | |
| | | | | cDNA | | | |
| 66 | Gma.15155.2.S1_a_at | 0.489 | BG789619 | Unclassified - Hypothetical | | | |
| | | | | Protein Supported by cDNA | | | |
| 67 | Gma.16441.1.S1_a_at | 0.489 | BU548048 | Unclassified - Hypothetical | | | HGG motif-containing thioesterase |
| | | | | Protein NOT Supported by | | | |
| | | | | cDNA | | | |
| 68 | Gma.1058.1.S1_at | 0.489 | BE820580 | Metabolism | | | |
| 69 | Gma.17305.1.S1_at | 0.489 | AW351319 | Unclassified - Protein with | | | |
| | | | | Unknown Function | | | |
| 70 | Gma.15729.1.A1_at | 0.489 | B1971055 | Cell Growth & Division | | | |
| 71 | GmaAffx.11840.1.S1_at | 0.488 | AW349211 | Post-Transcription | | | Predicted E3 ubiquitin ligase |
| 72 | GmaAffx.60958.3.S1_at | 0.487 | BG881523 | No Homology to Known | | | |
| | | | | Proteins | | | |
| 73 | GmaAffx.77269.1.S1_at | 0.487 | BM093698 | No Homology to Known | | | |
| | | | | Proteins | | | |
| 74 | Gma.7120.1.S1_at | 0.486 | BM093481 | Metabolism | | | Alanine-glyoxylate aminotransferase |
| | | | | | | | AGT2 |
| 75 | GmaAffx.6818.1.A1_at | 0.486 | BU546572 | Metabolism | | | Iron/ascorbate family oxidoreductases |
| 76 | GmaAffx.80638.1.S1_at | 0.486 | AW396070 | Unclassified - Hypothetical | | | |
| | | | | Protein NOT Supported by | | | |
| | | | | cDNA | | | |
| 77 | Gma.5621.1.S1_at | 0.485 | BU579234 | Unclassified - Hypothetical | | | |
| | | | | Protein NOT Supported by | | | |
| | | | | cDNA | | | |
| 78 | GmaAffx.93592.1.S1_s_at | 0.485 | CF809317 | Unclassified - Hypothetical | | | |
| | | | | Protein Supported by cDNA | | | |
| 79 | GmaAffx.77190.1.S1_at | 0.485 | BQ741890 | Metabolism | | | |
| 80 | GmaAffx.67456.1.S1_at | 0.485 | BM893057 | No Homology to Known | | | |
| | | | | Proteins | | | |
| 81 | GmaAffx.89049.1.S1_s_at | 0.485 | CK606168 | Intracellular Traffic | | | Annexin |
| 82 | GmaAffx.11419.1.S1_at | 0.485 | AI900831 | No Homology to Known | | | |
| | | | | Proteins | | | |
| 83 | GmaAffx.81692.1.S1_at | 0.484 | CA937041 | No Homology to Known | | | |
| | | | | Proteins | | | |
| 84 | Gma.197.1.S1_at | 0.484 | U55874.1 | Metabolism | Amino Acid | | Asparagine synthase (glutamine- |
| | | | | | Biosynthesis/ | | hydrolyzing) |
| | | | | | Metabolism | | |
| 85 | GmaAffx.87550.1.S1_at | 0.484 | BM954718 | Disease & Defense | | | |
| 86 | Gma.6803.2.S1_s_at | 0.483 | BU082740 | Disease & Defense | | | |
| 87 | GmaAffx.78790.1.S1_at | 0.483 | BQ298762 | Transporter | | | P-type ATPase |
| 88 | GmaAffx.91338.1.S1_at | 0.483 | CF807063 | Unclassified - Hypothetical | | | Predicted haloacid dehalogenase-like |
| | | | | Protein Supported by cDNA | | | hydrolase |
| 89 | GmaAffx.93534.1.S1_s_at | 0.483 | CF809259 | Unclassified - Protein with | | | |
| | | | | Unknown Function | | | |
| 90 | GmaAffx.47788.1.A1_at | 0.483 | CD408318 | Metabolism | | | Aldehyde dehydrogenase |
| 91 | Gma.10857.2.S1_s_at | 0.483 | BQ612778 | Unclassified - Hypothetical | | | Fasciclin and related adhesion |
| | | | | Protein Supported by cDNA | | | glycoproteins |
| 92 | GmaAffx.87721.1.S1_at | 0.483 | AW156448 | Intracellular Traffic | | | Rho GDP-dissociation inhibitor |
| 93 | Gma.13613.1.A1_at | 0.482 | CD401987 | No Homology to Known | | | |
| | | | | Proteins | | | |
| 94 | Gma.9013.1.S1_x_at | 0.482 | AF243368.1 | Disease & Defense | | | Glutathione S-transferase |
| 95 | Gma.3400.1.S1_at | 0.482 | BQ627988 | No Homology to Known | | | |
| | | | | Proteins | | | |
| 96 | Gma.10940.1.S1_at | 0.482 | BU550016 | Unclassified - Hypothetical | | | Metalloendopeptidase family - |
| | | | | Protein Supported by cDNA | | | saccharolysin & thimet oligopeptidase |
| 97 | GmaAffx.53033.1.S1_at | 0.482 | BG046837 | Transcription | Transcription | AP2/EREBP | |
| | | | | | Factor | | |
| 98 | GmaAffx.82263.1.S1_s_at | 0.482 | AW568454 | No Homology to Known | | | |
| | | | | Proteins | | | |
| 99 | Gma.5816.1.S1_at | 0.481 | B1787790 | Unclassified - Hypothetical | | | Aldehyde reductase |
| | | | | Protein Supported by cDNA | | | |
| 100 | Gma.2771.1.S1_at | 0.481 | AW309763 | Unclassified - Hypothetical | | | |
| | | | | Protein Supported by cDNA | | | |
| 101 | Gma.10701.2.S1_at | 0.481 | AW101512 | No Homology to Known | | | |
| | | | | Proteins | | | |
| 102 | GmaAffx.93260.1.S1_at | 0.481 | CF808985 | Signal Transduction | Protein Kinase | | |
| 103 | GmaAffx.53585.1.S1_at | 0.481 | B1471867 | Protein Destination & Storage | | | Molecular chaperone (DnaJ superfamily) |
| 104 | GmaAffx.77622.1.S1_at | 0.48 | B1674271 | Transcription | Transcription | Zinc Finger | |
| | | | | | Factor | | |
| 105 | GmaAffx.74868.1.S1_at | 0.48 | CA820573 | No Homology to Known | | | |
| | | | | Proteins | | | |
| 106 | GmaAffx.2412.1.S1_at | 0.48 | AI961002 | Secondary Metabolism | GA | | Iron/ascorbate family oxidoreductases |
| | | | | | Biosynthesis | | |
| 107 | GmaAffx.88251.1.S1_s_at | 0.48 | BU964415 | Disease & Defense | | | Soluble epoxide hydrolase |
| 108 | GmaAffx.68318.1.S1_at | 0.48 | BU545357 | Transcription | Transcription | AUX/1AA | |
| | | | | | Factor | | |
| 109 | Gma.17798.1.S1_at | 0.479 | BU084728 | Unclassified - Hypothetical | | | |
| | | | | Protein Supported by cDNA | | | |
| 110 | Gma.11334.1.S1_a_at | 0.479 | BI967327 | Cell Structure | | | O-methyltransferase |
| 111 | GmaAffx.49130.1.S1_at | 0.479 | BE555035 | No Homology to Known | | | |
| | | | | Proteins | | | |
| 112 | GmaAffx.37484.1.S1_at | 0.479 | BG651852 | Unclassified - Protein with | | | Predicted membrane protein, contains |
| | | | | Unknown Function | | | DoH and Cytochrome b-561/ferric |
| | | | | | | | reductase transmembrane domains |
| 113 | Gma.11341.3.S1_s_at | 0.479 | BM177332 | Unclassified - Hypothetical | | | |
| | | | | Protein NOT Supported by | | | |
| | | | | cDNA | | | |
| 114 | GmaAffx.47590.1.S1_at | 0.478 | CA853861 | Metabolism | | | UDP-glucuronosyl and UDP-glucosyl |
| | | | | | | | transferase |
| 115 | GmaAffx.499.1.S1_at | 0.478 | BE058375 | Transcription | Transcription | MYB | Transcription factor, Myb superfamily |
| | | | | | Factor | | |
| 116 | GmaAffx.90097.1.S1_at | 0.477 | CF805822 | Cell Structure | | | |
| 117 | Gma.15499.1.S1_at | 0.477 | AW348149 | Energy | | | |
| 118 | GmaAffx.83232.1.S1_at | 0.477 | BE023128 | Disease & Defense | | | |
| 119 | Gma.6075.1.S1_a_at | 0.476 | AW317183 | Disease & Defense | | | |
| 120 | Gma.3160.1.S1_at | 0.476 | BG507816 | Energy | | | Thioredoxin |
| 121 | Gma.8525.1.S1_s_at | 0.476 | AI966178 | Disease & Defense | | | |
| 122 | Gma.18052.1.S1_at | 0.476 | BF595464 | Transporter | | | |
| 123 | Gma.11672.1.S1_at | 0.476 | BF425929 | Unclassified - Hypothetical | | | |
| | | | | Protein Supported by cDNA | | | |
| 124 | Gma.385.1.S1_at | 0.475 | BE347563 | Intracellular Traffic | | | Annexin |
| 125 | Gma.11135.1.S1_at | 0.475 | AI856724 | Energy | | | ATP-citrate lyase |
| 126 | Gma.17070.1.S1_at | 0.475 | BE822463 | Disease & Defense | | | O-methyltransferase |
| 127 | Gma.1445.1.S1_a_at | 0.475 | BQ454241 | Metabolism | | | Flavonol reductase/cinnamoyl-CoA |
| | | | | | | | reductase |
| 128 | GmaAffx.26908.1.S1_at | 0.475 | BF425834 | Secondary Metabolism | | | Beta, beta-carotene 15,15′-dioxygenase |
| | | | | | | | and related enzymes |
| 129 | GmaAffx.35729.2.S1_at | 0.474 | BM732483 | Unclassified - Hypothetical | | | |
| | | | | Protein Supported by cDNA | | | |
| 130 | GmaAffx.77832.1.S1_at | 0.474 | BI971626 | Unclassified - Hypothetical | | | |
| | | | | Protein NOT Supported by | | | |
| | | | | cDNA | | | |
| 131 | Gma.13257.1.A1_at | 0.474 | CD396346 | Unclassified - Protein with | | | |
| | | | | Unknown Function | | | |
| 132 | Gma.9228.1.S1_at | 0.473 | CD417435 | Unclassified - Hypothetical | | | |
| | | | | Protein NOT Supported by | | | |
| | | | | cDNA | | | |
| 133 | Gma.1749.3.S1_at | 0.472 | B1784667 | Protein Destination & Storage | Ubiquitin | | FOG: Predicted E3 ubiquitin ligase |
| | | | | | Degradation | | |
| 134 | Gma.4643.1.S1_at | 0.472 | BU547906 | Transcription | Transcription | NAC Domain | |
| | | | | | Factor | | |
| 135 | GmaAffx.93212.1.S1_x_at | 0.472 | CF807524 | Transporter | | | |
| 136 | GmaAffx.45853.1.S1_at | 0.471 | BQ081229 | Unclassified - Protein with | | | |
| | | | | Unknown Function | | | |
| 137 | Gma.14563.1.S1_s_at | 0.471 | CD399224 | Energy | | | |
| 138 | GmaAffx.74500.1.A1_at | 0.471 | BU551117 | No Homology to Known | | | |
| | | | | Proteins | | | |
| 139 | Gma.16288.1.S1_at | 0.471 | AW598590 | Unclassified - Hypothetical | | | Fasciclin and related adhesion |
| | | | | Protein NOT Supported by | | | glycoproteins |
| | | | | cDNA | | | |
| 140 | GmaAffx.85342.1.S1_at | 0.471 | B1426916 | Unclassified - Hypothetical | | | |
| | | | | Protein NOT Supported by | | | |
| | | | | cDNA | | | |
| 141 | GmaAffx.24352.1.S1_at | 0.47 | BU544287 | No Homology to Known | | | |
| | | | | Proteins | | | |
| 142 | GmaAffx.15471.3.S1_s_at | 0.47 | BE346777 | Transcription | Transcription | MYB | Transcription factor, Myb superfamily |
| | | | | | Factor | | |
| 143 | Gma.17861.1.S1_at | 0.47 | AW310110 | Cell Structure | | | |
| 144 | Gma.1294.1.S1_at | 0.47 | CD395653 | Unclassified - Hypothetical | | | |
| | | | | Protein Supported by cDNA | | | |
| 145 | GmaAffx.36017.1.S1_at | 0.469 | BG154593 | Unclassified - Hypothetical | | | |
| | | | | Protein Supported by cDNA | | | |
| 146 | GmaAffx.83638.1.S1_at | 0.469 | BU549896 | Unclassified - Hypothetical | | | |
| | | | | Protein NOT Supported by | | | |
| | | | | cDNA | | | |
| 147 | Gma.2470.1.S1_s_at | 0.468 | AI442629 | Cell Structure | | | |
| 148 | GmaAffx.91514.1.S1_at | 0.468 | CF807239 | No Homology to Known | | | |
| | | | | Proteins | | | |
| 149 | GmaAffx.28690.1.S1_at | 0.467 | CA799411 | Transcription | Transcription | Homeodomain | |
| | | | | | Factor | | |
| 150 | Gma.17763.1.A1_at | 0.467 | B1321757 | Unclassified - Hypothetical | | | Diadenosine and diphosphoinositol |
| | | | | Protein Supported by cDNA | | | polyphosphate phosphohydrolase |
| 151 | Gma.6659.1.S1_at | 0.467 | BQ629137 | Unclassified - Protein with | | | Protein involved in membrane traffic |
| | | | | Unknown Function | | | (YOP1/TB2/DP1/HVA22 family) |
| 152 | Gma.8143.1.S1_at | 0.466 | BG725425 | Signal Transduction | MAPK | | Mitogen-activated protein kinase |
| 153 | GmaAffx.68099.1.S1_at | 0.466 | AW734055 | No Homology to Known | | | |
| | | | | Proteins | | | |
| 154 | Gma.3065.1.S1_s_at | 0.466 | AW349564 | Unclassified - Hypothetical | | | |
| | | | | Protein Supported by cDNA | | | |
| 155 | GmaAffx.87526.1.S1_at | 0.465 | BM954581 | Unclassified - Hypothetical | | | |
| | | | | Protein Supported by cDNA | | | |
| 156 | Gma.3881.1.S1_at | 0.465 | X92437.1 | Metabolism | Cytochrome | CYP73A | Cytochrome P450 CYP2 subfamily |
| | | | | | P450 | | |
| 157 | Gma.5468.2.S1_a_at | 0.465 | BE802025 | Metabolism | | | Beta-galactosidase |
| 158 | GmaAffx.87295.1.S1_at | 0.465 | BM519660 | No Homology to Known | | | |
| | | | | Proteins | | | |
| 159 | Gma.1701.1.S1_at | 0.464 | M67449.1 | Signal Transduction | MAPKKK | Raf | Tyrosine kinase specific for activated |
| | | | | | | | (GTP-bound) p21cdc42Hs |
| 160 | GmaAffx.3548.1.S1_at | 0.464 | CA851347 | Transporter | | | Apolipoprotein D/Lipocalin |
| 161 | Gma.12584.2.A1_at | 0.464 | CD406878 | Metabolism | | | Long-chain acyl-CoA synthetases (AMP- |
| | | | | | | | forming) |
| 162 | GmaAffx.8113.1.S1_at | 0.463 | BE659323 | Disease & Defense | | | |
| 163 | GmaAffx.64885.1.S1_at | 0.462 | BQ081653 | No Homology to Known | | | |
| | | | | Proteins | | | |
| 164 | Gma.1445.3.S1_a_at | 0.461 | BE805686 | Metabolism | | | Flavonol reductase/cinnamoyl-CoA |
| | | | | | | | reductase |
| 165 | GmaAffx.93308.1.S1_at | 0.461 | CF809033 | Protein Destination & Storage | | | Glutathione S-transferase |
| 166 | Gma.8072.1.S1_a_at | 0.461 | BQ627840 | Transcription | Transcription | Basic Leucine | |
| | | | | | Factor | Zipper (bZIP) | |
| 167 | GmaAffx.68588.1.S1_at | 0.46 | B1787152 | Unclassified - Hypothetical | | | |
| | | | | Protein Supported by cDNA | | | |
| 168 | GmaAffx.69369.1.S1_at | 0.46 | BG511072 | Unclassified - Hypothetical | | | Galactosyltransferases |
| | | | | Protein Supported by cDNA | | | |
| 169 | GmaAffx.21943.1.S1_at | 0.46 | AW101735 | Unclassified - Hypothetical | | | |
| | | | | Protein NOT Supported by | | | |
| | | | | cDNA | | | |
| 170 | GmaAffx.24382.1.S1_at | 0.46 | BM886764 | No Homology to Known | | | |
| | | | | Proteins | | | |
| 171 | GmaAffx.64794.1.S1_at | 0.46 | CA801749 | Unclassified - Hypothetical | | | |
| | | | | Protein Supported by cDNA | | | |
| 172 | GmaAffx.62941.1.S1_at | 0.459 | AW597498 | Unclassified - Protein with | | | |
| | | | | Unknown Function | | | |
| 173 | GmaAffx.80474.1.S1_at | 0.458 | AW707170 | Signal Transduction | MAPKKK | MEKK | MEKK and related serine/threonine |
| | | | | | | | protein kinases |
| 174 | GmaAffx.59210.1.S1_at | 0.458 | B1967182 | Secondary Metabolism | Ethylene | | Iron/ascorbate family oxidoreductases |
| | | | | | Biosynthesis | | |
| 175 | GmaAffx.84721.2.S1_s_at | 0.458 | BG041982 | Metabolism | | | Hydroxymethylglutaryl-CoA lyase |
| 176 | GmaAffx.53069.1.S1_at | 0.458 | AW733700 | Unclassified - Hypothetical | | | |
| | | | | Protein NOT Supported by | | | |
| | | | | cDNA | | | |
| 177 | GmaAffx.69076.1.S1_at | 0.458 | BM525610 | Unclassified - Hypothetical | | | |
| | | | | Protein NOT Supported by | | | |
| | | | | cDNA | | | |
| 178 | GmaAffx.18236.1.S1_at | 0.458 | BG790288 | No Homology to Known | | | |
| | | | | Proteins | | | |
| 179 | Gma.13458.1.A1_at | 0.458 | CD398733 | Protein Destination & Storage | Ubiquitin | | FOG: Predicted E3 ubiquitin ligase |
| | | | | | Degradation | | |
| 180 | Gma.16896.1.A1_at | 0.457 | CD416188 | No Homology to Known | | | |
| | | | | Proteins | | | |
| 181 | GmaAffx.15204.1.A1_at | 0.457 | BU549835 | No Homology to Known | | | |
| | | | | Proteins | | | |
| 182 | Gma.412.1.S1_at | 0.456 | BE607835 | Protein Destination & Storage | | | Ubiquitin-protein ligase |
| 183 | Gma.2253.1.S1_at | 0.456 | AF169018.1 | Metabolism | | | Reductases with broad range of substrate |
| | | | | | | | specificities |
| 184 | GmaAffx.51044.1.S1_at | 0.456 | BM522670 | Unclassified - Hypothetical | | | Copine |
| | | | | Protein Supported by cDNA | | | |
| 185 | Gma.10520.1.A1_at | 0.456 | CD398227 | Unclassified - Hypothetical | | | |
| | | | | Protein Supported by cDNA | | | |
| 186 | GmaAffx.85565.1.S1_at | 0.456 | BE611082 | Signal Transduction | Calcium | Calcium | Ca2+/calmodulin-dependent protein |
| | | | | | Binding | Dependent | kinase, EF-Hand protein superfamily |
| | | | | | | Protein Kinase | |
| 187 | GmaAffx.78768.1.S1_s_at | 0.456 | CA784898 | No Homology to Known | | | |
| | | | | Proteins | | | |
| 188 | Psojae_rRNA_482_at | 0.455 | | | | | |
| 189 | Gma.14611.1.S1_at | 0.454 | CA852673 | Unclassified - Protein with | | | |
| | | | | Unknown Function | | | |
| 190 | GmaAffx.86745.1.S1_s_at | 0.454 | B1424960 | Disease & Defense | | | |
| 191 | GmaAffx.50617.1.S1_at | 0.454 | AW101420 | Unclassified - Hypothetical | | | |
| | | | | Protein Supported by cDNA | | | |
| 192 | Gma.4374.1.S1_at | 0.454 | BM178538 | Metabolism | Cytochrome | CYP76C | Cytochrome P450 CYP2 subfamily |
| | | | | | P450 | | |
| 193 | GmaAffx.93017.1.S1_at | 0.454 | CF808742 | Disease & Defense | | | Soluble epoxide hydrolase |
| 194 | Gma.4673.1.A1_at | 0.453 | BG508468 | No Homology to Known | | | |
| | | | | Proteins | | | |
| 195 | GmaAffx.21773.1.S1_at | 0.453 | BU763882 | Unclassified - Hypothetical | | | |
| | | | | Protein NOT Supported by | | | |
| | | | | cDNA | | | |
| 196 | GmaAffx.24521.1.S1_at | 0.453 | BU761767 | Unclassified - Hypothetical | | | |
| | | | | Protein NOT Supported by | | | |
| | | | | cDNA | | | |
| 197 | Gma.15563.1.S1_at | 0.453 | CD415760 | Unclassified - Hypothetical | | | |
| | | | | Protein Supported by cDNA | | | |
| 198 | Gma.5513.1.S1_x_at | 0.453 | AW310453 | Metabolism | | | Haloacid dehalogenase-like hydrolase |
| 199 | GmaAffx.10297.1.S1_at | 0.453 | BE475510 | Metabolism | | | Beta-galactosidase |
| 200 | Gma.3804.1.A1_at | 0.452 | AW310249 | Disease & Defense | | | |
| 201 | Gma.9842.1.S1_at | 0.452 | BM528240 | Unclassified - Hypothetical | | | Leucine rich repeat proteins, some |
| | | | | Protein Supported by cDNA | | | proteins contain F-box |
| 202 | GmaAffx.18113.2.S1_x_at | 0.452 | AW706795 | Unclassified - Protein with | Hormone | | |
| | | | | Unknown Function | Induced | | |
| 203 | GmaAffx.90853.1.S1_at | 0.452 | CF806578 | No Homology to Known | | | |
| | | | | Proteins | | | |
| 204 | GmaAffx.54778.1.S1_at | 0.451 | B1322039 | No Homology to Known | | | |
| | | | | Proteins | | | |
| 205 | Gma.13042.1.S1_at | 0.45 | CD390874 | Unclassified - Hypothetical | | | |
| | | | | Protein NOT Supported by | | | |
| | | | | cDNA | | | |
| 206 | Gma.6804.1.S1_at | 0.449 | BE657581 | Protein Destination & Storage | | | Aspartyl protease |
| 207 | Gma.17136.1.S1_s_at | 0.449 | BE022719 | Energy | | | Cytochrome b |
| 208 | GmaAffx.5264.1.S1_s_at | 0.448 | BM521512 | Disease & Defense | | | |
| 209 | GmaAffx.92463.1.S1_at | 0.448 | CF808188 | Metabolism | | | NADP+-dependent malic enzyme |
| 210 | GmaAffx.20688.1.S1_at | 0.448 | BG157357 | Unclassified - Protein with | | | Fatty acyl-CoA |
| | | | | Unknown Function | | | elongase/Polyunsaturated fatty acid |
| | | | | | | | specific elongation enzyme |
| 211 | GmaAffx.81388.1.S1_at | 0.448 | BF424571 | Signal Transduction | Protein Kinase | | Serine/threonine protein kinase |
| 212 | GmaAffx.11892.1.S1_at | 0.447 | BE820605 | Secondary Metabolism | | | Iron/ascorbate family oxidoreductases |
| 213 | GmaAffx.6017.2.S1_at | 0.446 | B1469662 | Unclassified - Hypothetical | | | |
| | | | | Protein NOT Supported by | | | |
| | | | | cDNA | | | |
| 214 | GmaAffx.91688.1.S1_at | 0.446 | CF807413 | No Homology to Known | | | |
| | | | | Proteins | | | |
| 215 | Gma.3420.1.S1_at | 0.445 | CD406082 | Unclassified - Hypothetical | | | |
| | | | | Protein Supported by cDNA | | | |
| 216 | GmaAffx.35278.1.S1_at | 0.445 | BE346991 | Disease & Defense | | | |
| 217 | GmaAffx.88786.1.A1_s_at | 0.444 | CK606552 | Cell Structure | | | O-methyltransferase |
| 218 | GmaAffx.73088.1.S1_at | 0.444 | BE475110 | Unclassified - Hypothetical | | | |
| | | | | Protein Supported by cDNA | | | |
| 219 | Gma.15487.1.S1_at | 0.444 | D63781.1 | Disease & Defense | | | Soluble epoxide hydrolase |
| 220 | Gma.4305.3.S1_a_at | 0.444 | BG044173 | Transporter | 1on Channel | Cyclic nucleotide | K+-channel ERG and related proteins, |
| | | | | | Families | gated channel | contain PAS/PAC sensor domain |
| | | | | | | family | |
| 221 | GmaAffx.77097.1.S1_at | 0.443 | BG237643 | No Homology to Known | | | |
| | | | | Proteins | | | |
| 222 | GmaAffx.18351.1.S1_at | 0.443 | B1472051 | Unclassified - Hypothetical | | | FOG: Armadillo/beta-catenin-like |
| | | | | Protein Supported by cDNA | | | repeats |
| 223 | Gma.4437.1.S1_s_at | 0.443 | BQ628831 | Secondary Metabolism | Flavonoid | | Flavonol reductase/cinnamoyl-CoA |
| | | | | | Biosynthesis | | reductase |
| 224 | GmaAffx.18599.1.S1_at | 0.443 | B1787464 | Secondary Metabolism | Amines | | Amine oxidase |
| | | | | | Biosynthesis | | |
| 225 | Gma.5418.2.S1_at | 0.442 | B1469627 | Cell Growth & Division | | | |
| 226 | Gma.5218.1.A1_at | 0.442 | CD392232 | Metabolism | | | Predicted carbonic anhydrase involved |
| | | | | | | | in protection against oxidative damage |
| 227 | GmaAffx.84795.1.S1_at | 0.442 | BG046191 | Unclassified - Hypothetical | | | |
| | | | | Protein NOT Supported by | | | |
| | | | | cDNA | | | |
| 228 | Gma.415.1.A1_at | 0.442 | AW350315 | Protein Destination & Storage | | | FOG: Predicted E3 ubiquitin ligase |
| 229 | GmaAffx.50980.1.S1_at | 0.442 | BE823095 | Signal Transduction | Protein | | |
| | | | | | Phosphatase | | |
| 230 | Gma.1123.1.S1_at | 0.441 | CD402207 | Unclassified - Hypothetical | | | |
| | | | | Protein Supported by cDNA | | | |
| 231 | GmaAffx.44484.1.S1_at | 0.441 | BE475213 | Unclassified - Hypothetical | | | |
| | | | | Protein NOT Supported by | | | |
| | | | | cDNA | | | |
| 232 | GmaAffx.26683.1.A1_at | 0.44 | BE820379 | No Homology to Known | | | |
| | | | | Proteins | | | |
| 233 | Gma.17798.2.S1_at | 0.44 | CD412748 | No Homology to Known | | | |
| | | | | Proteins | | | |
| 234 | GmaAffx.93430.1.S1_s_at | 0.439 | CF809155 | Disease & Defense | | | Purple (tartrate-resistant) acid |
| | | | | | | | phosphatase |
| 235 | GmaAffx.34775.1.S1_at | 0.439 | AI794730 | Unclassified - Protein with | | | FOG: Predicted E3 ubiquitin ligase |
| | | | | Unknown Function | | | |
| 236 | GmaAffx.89772.8.S1_x_at | 0.439 | CK605778 | Metabolism | | | |
| 237 | Gma.9259.1.S1_at | 0.439 | CA782161 | Transporter | | | |
| 238 | Gma.5513.1.S1_at | 0.438 | AW310453 | Metabolism | | | Haloacid dehalogenase-like hydrolase |
| 239 | GmaAffx.17961.2.S1_at | 0.438 | BG511562 | Protein Destination & Storage | | | Molecular chaperones HSP70/HSC70, |
| | | | | | | | HSP70 superfamily |
| 240 | GmaAffx.9482.2.S1_at | 0.438 | AI900615 | Unclassified - Hypothetical | | | Predicted guanosine polyphosphate |
| | | | | Protein NOT Supported by | | | pyrophosphohydrolase/synthase |
| | | | | cDNA | | | |
| 241 | Gma.3589.1.S1_a_at | 0.437 | AW101438 | Secondary Metabolism | GA | | Iron/ascorbate family oxidoreductases |
| | | | | | Biosynthesis | | |
| 242 | Gma.6245.1.S1_at | 0.437 | CA938361 | No Homology to Known | | | |
| | | | | Proteins | | | |
| 243 | Gma.2486.1.S1_at | 0.437 | BF219523 | Unclassified - Hypothetical | | | Iron/ascorbate family oxidoreductases |
| | | | | Protein NOT Supported by | | | |
| | | | | cDNA | | | |
| 244 | Gma.12494.1.S1_at | 0.437 | BG839922 | Protein Destination & Storage | Ubiquitin | E3 Ubiquitin | FOG: Predicted E3 ubiquitin ligase |
| | | | | | Degradation | Ligase | |
| 245 | GmaAffx.51468.1.S1_at | 0.437 | BQ628796 | No Homology to Known | | | |
| | | | | Proteins | | | |
| 246 | Gma.17062.1.S1_at | 0.436 | AW277440 | No Homology to Known | | | |
| | | | | Proteins | | | |
| 247 | GmaAffx.53099.1.S1_at | 0.436 | BG043340 | Unclassified - Hypothetical | | | Alpha-L-fucosidase |
| | | | | Protein NOT Supported by | | | |
| | | | | cDNA | | | |
| 248 | GmaAffx.78163.1.S1_s_at | 0.436 | BM520857 | Signal Transduction | Protein | | Serine/threonine protein phosphatase |
| | | | | | Phosphatase | | |
| 249 | GmaAffx.58073.1.S1_at | 0.436 | CA852105 | Unclassified - Hypothetical | | | |
| | | | | Protein Supported by cDNA | | | |
| 250 | Gma.892.1.A1_at | 0.435 | BE822836 | Unclassified - Protein with | | | Prolyl 4-hydroxylase alpha subunit |
| | | | | Unknown Function | | | |
| 251 | GmaAffx.74304.1.S1_at | 0.435 | BU577360 | Unclassified - Hypothetical | | | |
| | | | | Protein NOT Supported by | | | |
| | | | | cDNA | | | |
| 252 | GmaAffx.81253.1.S1_at | 0.434 | BE807371 | Unclassified - Protein with | | | |
| | | | | Unknown Function | | | |
| 253 | Gma.18084.1.S1_at | 0.434 | BI317557 | Disease & Defense | | | Ferric reductase, NADH/NADPH |
| | | | | | | | oxidase and related proteins |
| 254 | GmaAffx.91108.1.S1_at | 0.434 | CF806833 | Unclassified - Hypothetical | | | |
| | | | | Protein Supported by cDNA | | | |
| 255 | Gma.1190.1.S1_at | 0.434 | AF279267.1 | Disease & Defense | | | Acyl-CoA synthetase |
| 256 | GmaAffx.92192.1.S1_s_at | 0.434 | CF806406 | Disease & Defense | | | |
| 257 | Gma.10281.1.S1_at | 0.433 | CA852560 | Unclassified - Protein with | | | Predicted E3 ubiquitin ligase |
| | | | | Unknown Function | | | |
| 258 | Gma.4315.1.S1_at | 0.433 | BM886786 | Unclassified - Protein with | | | |
| | | | | Unknown Function | | | |
| 259 | GmaAffx.71944.1.S1_at | 0.433 | AW351155 | Unclassified - Hypothetical | | | |
| | | | | Protein Supported by cDNA | | | |
| 260 | GmaAffx.91814.1.S1_at | 0.432 | CF807539 | Unclassified - Hypothetical | | | |
| | | | | Protein Supported by cDNA | | | |
| 261 | GmaAffx.84751.1.A1_at | 0.432 | AW349702 | Transcription | Transcription | MYB | Transcription factor, Myb superfamily |
| | | | | | Factor | | |
| 262 | Gma.15773.1.A1_at | 0.431 | CD406280 | Metabolism | | | |
| 263 | Gma.12873.1.S1_at | 0.431 | CD393774 | No Homology to Known | | | |
| | | | | Proteins | | | |
| 264 | GmaAffx.9926.2.S1_at | 0.431 | BF597587 | Unclassified - Hypothetical | | | |
| | | | | Protein Supported by cDNA | | | |
| 265 | Gma.14312.1.S1_at | 0.431 | CA852635 | No Homology to Known | | | |
| | | | | Proteins | | | |
| 266 | Gma.15470.1.S1_at | 0.43 | BU546303 | Unclassified - Hypothetical | | | |
| | | | | Protein Supported by cDNA | | | |
| 267 | GmaAffx.15222.1.S1_at | 0.43 | BU550700 | Unclassified - Hypothetical | | | |
| | | | | Protein NOT Supported by | | | |
| | | | | cDNA | | | |
| 268 | GmaAffx.4505.1.S1_at | 0.43 | AW306682 | Unclassified - Protein with | | | Predicted NUDIX hydrolase FGF-2 and |
| | | | | Unknown Function | | | related proteins |
| 268 | GmaAffx.4505.1.S1_at | 0.43 | AW306682 | Unclassified - Protein with | | | Cdc4 and related F-box and WD-40 |
| | | | | Unknown Function | | | proteins |
| 269 | GmaAffx.90827.1.S1_at | 0.43 | CF806552 | Unclassified - Hypothetical | | | Molecular chaperone (small heat-shock |
| | | | | Protein Supported by cDNA | | | protein Hsp26/Hsp42) |
| 270 | GmaAffx.92184.1.S1_s_at | 0.43 | CF807909 | Unclassified - Protein with | | | |
| | | | | Unknown Function | | | |
| 271 | GmaAffx.93592.1.S1_x_at | 0.429 | CF809317 | Unclassified - Hypothetical | | | |
| | | | | Protein Supported by cDNA | | | |
| 272 | GmaAffx.89971.1.S1_s_at | 0.429 | CF805696 | Intracellular Traffic | | | Annexin |
| 273 | GmaAffx.66982.1.S1_at | 0.428 | BI970847 | Protein Destination & Storage | | | Molecular chaperone (small heat-shock |
| | | | | | | | protein Hsp26/Hsp42) |
| 274 | GmaAffx.45236.1.S1_at | 0.428 | BG156638 | Disease & Defense | | | Purple (tartrate-resistant) acid |
| | | | | | | | phosphatase |
| 275 | GmaAffx.79888.1.S1_at | 0.428 | AI988123 | Signal Transduction | Protein Kinase | | |
| 276 | Gma.6075.1.S1_at | 0.428 | AW317183 | Disease & Defense | | | |
| 277 | Gma.10795.1.S1_at | 0.428 | AI442845 | Cell Structure | | | |
| 278 | GmaAffx.55244.1.A1_at | 0.427 | BU549116 | No Homology to Known | | | |
| | | | | Proteins | | | |
| 279 | GmaAffx.69792.1.S1_s_at | 0.427 | BU550596 | Transcription | Transcription | AP2/EREBP | |
| | | | | | Factor | | |
| 280 | GmaAffx.30428.1.S1_at | 0.427 | BE824410 | Protein Destination & Storage | | | Molecular chaperones HSP70/HSC70, |
| | | | | | | | HSP70 superfamily |
| 281 | GmaAffx.90104.1.S1_s_at | 0.427 | CF805829 | Transporter | | | Permease of the major facilitator |
| | | | | | | | superfamily |
| 282 | Gma.3969.2.S1_at | 0.426 | CD412522 | Energy | | | |
| 283 | Gma.16547.1.S1_at | 0.426 | BU545050 | Transcription | Transcription | WRKY | |
| | | | | | Factor | | |
| 284 | GmaAffx.74211.1.S1_at | 0.426 | BE820853 | Protein Destination & Storage | | | Aspartyl protease |
| 285 | GmaAffx.42667.3.S1_at | 0.426 | AW306785 | Transcription | Transcription | GRAS | |
| | | | | | Factor | | |
| 286 | Gma.2772.1.S1_at | 0.426 | BG238134 | No Homology to Known | | | |
| | | | | Proteins | | | |
| 287 | Gma.5889.1.S1_at | 0.425 | BQ610202 | Unclassified - Hypothetical | | | Endoplasmic reticulum protein EP58, |
| | | | | Protein NOT Supported by | | | contains filamin rod domain and KDEL |
| | | | | cDNA | | | motif |
| 288 | GmaAffx.18742.1.S1_at | 0.425 | B1970450 | Unclassified - Hypothetical | | | Predicted membrane protein |
| | | | | Protein Supported by cDNA | | | |
| 289 | Gma.1947.1.S1_at | 0.425 | BI968172 | Transcription | Transcription | Homeodomain | Transcription factor MEIS1 and related |
| | | | | | Factor | | HOX domain proteins |
| 290 | Gma.5400.1.S1_at | 0.425 | BM093775 | No Homology to Known | | | |
| | | | | Proteins | | | |
| 291 | Gma.6709.1.S1_at | 0.424 | BE823291 | Signal Transduction | Protein Kinase | AtC1PKs | Serine/threonine protein kinase |
| 292 | Gma.11131.1.S1_at | 0.424 | B1968388 | Protein Destination & Storage | | | Molecular chaperone (small heat-shock |
| | | | | | | | protein Hsp26/Hsp42) |
| 293 | GmaAffx.72580.1.S1_at | 0.424 | BG157182 | Unclassified - Hypothetical | | | |
| | | | | Protein Supported by cDNA | | | |
| 294 | Gma.4432.1.S1_a_at | 0.423 | BQ610841 | Unclassified - Protein with | Hormone | | Voltage-gated shaker-like K+ channel, |
| | | | | Unknown Function | Induced | | subunit beta/KCNAB |
| 295 | GmaAffx.74194.1.S1_at | 0.423 | B1968058 | Metabolism | | | 17 beta-hydroxysteroid dehydrogenase |
| | | | | | | | type 3, HSD17B3 |
| 296 | GmaAffx.62375.1.S1_at | 0.423 | BQ612727 | Unclassified - Hypothetical | | | |
| | | | | Protein Supported by cDNA | | | |
| 297 | GmaAffx.38733.1.A1_at | 0.422 | BE657590 | No Homology to Known | | | |
| | | | | Proteins | | | |
| 298 | GmaAffx.65580.1.S1_at | 0.422 | CA799171 | Metabolism | | | |
| 299 | GmaAffx.81618.1.S1_at | 0.421 | AW706403 | No Homology to Known | | | |
| | | | | Proteins | | | |
| 300 | GmaAffx.16201.1.S1_at | 0.421 | BU550725 | No Homology to Known | | | |
| | | | | Proteins | | | |
| 301 | GmaAffx.55124.1.S1_at | 0.421 | BI970821 | Unclassified - Hypothetical | | | Metacaspase involved in regulation of |
| | | | | Protein Supported by cDNA | | | apoptosis |
| 302 | Gma.4755.1.S1_at | 0.421 | AW310232 | Unclassified - Hypothetical | | | |
| | | | | Protein NOT Supported by | | | |
| | | | | cDNA | | | |
| 303 | GmaAffx.9059.1.S1_at | 0.421 | AW423327 | No Homology to Known | | | |
| | | | | Proteins | | | |
| 304 | GmaAffx.51614.1.S1_at | 0.42 | BU089837 | No Homology to Known | | | |
| | | | | Proteins | | | |
| 305 | Gma.153.1.S1_x_at | 0.42 | Y10491.1 | Metabolism | Cytochrome | CYP82C | Cytochrome P450 CYP2 subfamily |
| | | | | | P450 | | |
| 306 | GmaAffx.50980.1.S1_x_at | 0.419 | BE823095 | Signal Transduction | Protein | | |
| | | | | | Phosphatase | | |
| 307 | GmaAffx.47716.1.A1_at | 0.418 | CD397037 | Unclassified - Hypothetical | | | |
| | | | | Protein NOT Supported by | | | |
| | | | | cDNA | | | |
| 308 | Gma.4680.1.A1_s_at | 0.418 | AW309707 | Unclassified - Protein with | | | |
| | | | | Unknown Function | | | |
| 309 | Gma.17288.1.A1_at | 0.418 | AW310692 | No Homology to Known | | | |
| | | | | Proteins | | | |
| 310 | Gma.5258.1.S1_at | 0.418 | BE658899 | No Homology to Known | | | |
| | | | | Proteins | | | |
| 311 | GmaAffx.2710.1.S1_at | 0.417 | BE822538 | Unclassified - Hypothetical | | | Endoplasmic reticulum protein EP58, |
| | | | | Protein Supported by cDNA | | | contains filamin rod domain and KDEL |
| | | | | | | | motif |
| 312 | GmaAffx.89049.1.S1_at | 0.417 | CK606168 | Intracellular Traffic | | | Annexin |
| 313 | Gma.6046.1.S1_at | 0.417 | BE022758 | Unclassified - Hypothetical | | | |
| | | | | Protein NOT Supported by | | | |
| | | | | cDNA | | | |
| 314 | Gma.4839.1.A1_at | 0.417 | AW310019 | Metabolism | | | Branched chain aminotransferase |
| | | | | | | | BCAT1, pyridoxal phosphate enzymes |
| | | | | | | | type IV superfamily |
| 315 | GmaAffx.26320.1.S1_at | 0.416 | BE440416 | Unclassified - Hypothetical | | | |
| | | | | Protein Supported by cDNA | | | |
| 316 | Gma.10969.1.S1_x_at | 0.416 | U36191.1 | Metabolism | | | |
| 317 | GmaAffx.90234.1.S1_at | 0.416 | CF805959 | Transcription | Transcription | Zinc Finger | FOG: Zn-finger |
| | | | | | Factor | | |
| 318 | GmaAffx.5232.1.S1_at | 0.416 | BM309521 | Unclassified - Protein with | | | |
| | | | | Unknown Function | | | |
| 319 | GmaAffx.28578.1.S1_at | 0.416 | BQ094224 | Disease & Defense | | | |
| 320 | Gma.1326.1.S1_at | 0.416 | CD397515 | Secondary Metabolism | | | |
| 321 | GmaAffx.59762.1.S1_at | 0.416 | BI971811 | Unclassified - Protein with | | | |
| | | | | Unknown Function | | | |
| 322 | Gma.1715.1.S1_at | 0.415 | BE191618 | Metabolism | | | |
| 323 | Gma.18020.1.S1_at | 0.415 | AW309711 | Signal Transduction | Protein Kinase | | Serine/threonine protein kinase |
| 324 | GmaAffx.10580.1.S1_at | 0.414 | BF424010 | Transporter | | | Amino acid transporters |
| 325 | GmaAffx.12369.1.A1_at | 0.414 | CD412820 | Unclassified - Hypothetical | | | |
| | | | | Protein Supported by cDNA | | | |
| 326 | Gma.5256.2.S1_at | 0.414 | AI441316 | Unclassified - Hypothetical | | | |
| | | | | Protein NOT Supported by | | | |
| | | | | cDNA | | | |
| 327 | GmaAffx.1890.1.S1_s_at | 0.414 | B1970941 | Transcription | Transcription | Heat Shock | Heat shock transcription factor |
| | | | | | Factor | | |
| 328 | Gma.10266.1.S1_at | 0.414 | BG157207 | No Homology to Known | | | |
| | | | | Proteins | | | |
| 329 | Gma.17535.1.S1_at | 0.413 | BM178659 | Unclassified - Hypothetical | | | Uncharacterized conserved protein, |
| | | | | Protein Supported by cDNA | | | contains double-stranded beta-helix |
| | | | | | | | domain |
| 330 | Gma.4359.1.S1_at | 0.413 | AI794712 | Energy | | | Thioredoxin |
| 331 | Gma.7836.2.S1_at | 0.413 | B1425718 | Unclassified - Hypothetical | | | |
| | | | | Protein NOT Supported by | | | |
| | | | | cDNA | | | |
| 332 | Gma.17502.2.S1_at | 0.413 | BG046201 | Transcription | Transcription | Unclassified | Zuotin and related molecular chaperones |
| | | | | | Factor | | (DnaJ superfamily), contains DNA- |
| | | | | | | | binding domains |
| 333 | Gma.4839.2.S1_at | 0.412 | B1974385 | Metabolism | | | Branched chain aminotransferase |
| | | | | | | | BCAT1, pyridoxal phosphate enzymes |
| | | | | | | | type 1V superfamily |
| 334 | Gma.5162.1.A1_at | 0.412 | B1971156 | Signal Transduction | Protein Kinase | | |
| 335 | Gma.11195.1.S1_at | 0.412 | CD417406 | Transporter | | | Apolipoprotein D/Lipocalin |
| 336 | Gma.11028.1.S1_at | 0.412 | BE823084 | Metabolism | | | Glyoxylate/hydroxypyruvate reductase |
| | | | | | | | (D-isomer-specific 2-hydroxy acid |
| | | | | | | | dehydrogenase superfamily) |
| 337 | Gma.16521.1.S1_at | 0.412 | BE609300 | Unclassified - Hypothetical | | | Uncharacterized conserved protein |
| | | | | Protein Supported by cDNA | | | |
| 338 | Gma.7824.1.S1_at | 0.412 | BU550492 | Metabolism | | | Kynurenine 3-monooxygenase and |
| | | | | | | | related flavoprotein monooxygenases |
| 339 | GmaAffx.76346.1.A1_at | 0.412 | BQ612378 | Energy | | | 1,4-benzoquinone reductase-like; Trp |
| | | | | | | | repressor binding protein-like/protoplast- |
| | | | | | | | secreted protein |
| 340 | GmaAffx.91108.1.S1_s_at | 0.411 | CF806833 | Unclassified - Hypothetical | | | |
| | | | | Protein Supported by cDNA | | | |
| 341 | Gma.5610.1.S1_at | 0.41 | AW348190 | Unclassified - Hypothetical | | | |
| | | | | Protein Supported by cDNA | | | |
| 342 | GmaAffx.60673.1.S1_at | 0.409 | BU080770 | Unclassified - Hypothetical | | | |
| | | | | Protein NOT Supported by | | | |
| | | | | cDNA | | | |
| 343 | Gma.16477.1.S1_at | 0.409 | BE824134 | No Homology to Known | | | |
| | | | | Proteins | | | |
| 344 | Gma.4455.2.S1_at | 0.409 | BE059560 | Signal Transduction | Protein Kinase | | Serine/threonine protein kinase |
| 345 | Gma.10581.1.S1_at | 0.409 | AW396439 | Unclassified - Hypothetical | | | Predicted haloacid dehalogenase-like |
| | | | | Protein Supported by cDNA | | | hydrolase |
| 346 | GmaAffx.26299.1.S1_at | 0.408 | BQ299016 | Signal Transduction | Protein Kinase | | |
| 347 | Gma.13241.1.S1_at | 0.408 | CD395739 | Unclassified - Hypothetical | | | |
| | | | | Protein NOT Supported by | | | |
| | | | | cDNA | | | |
| 348 | Gma.7120.2.S1_a_at | 0.408 | BG041285 | Metabolism | | | Alanine-glyoxylate aminotransferase |
| | | | | | | | AGT2 |
| 349 | Gma.7227.1.S1_at | 0.408 | BU551112 | Protein Destination & Storage | | | Ubiquitin-protein ligase |
| 350 | GmaAffx.57970.1.S1_at | 0.407 | BM886862 | Transcription | Transcription | NAC Domain | |
| | | | | | Factor | | |
| 351 | Gma.17806.1.S1_at | 0.406 | BE820775 | Transporter | | | H+/oligopeptide symporter |
| 352 | Gma.14295.1.S1_at | 0.405 | CA852253 | No Homology to Known | | | |
| | | | | Proteins | | | |
| 353 | Gma.6761.1.A1_at | 0.405 | BQ453407 | Cell Structure | | | Beta-glucosidase, lactase |
| | | | | | | | phlorizinhydrolase, and related proteins |
| 354 | GmaAffx.26859.1.S1_s_at | 0.405 | BM085620 | Disease & Defense | | | Glutathione S-transferase |
| 355 | GmaAffx.83564.1.S1_at | 0.405 | BU578309 | No Homology to Known | | | |
| | | | | Proteins | | | |
| 356 | Gma.17642.1.S1_at | 0.405 | BU550265 | Unclassified - Hypothetical | | | BP1/LBP/CETP family protein |
| | | | | Protein Supported by cDNA | | | |
| 357 | GmaAffx.73303.1.S1_at | 0.405 | BE190156 | Unclassified - Hypothetical | | | Predicted membrane protein |
| | | | | Protein Supported by cDNA | | | |
| 358 | Gma.3283.1.S1_at | 0.404 | BQ298733 | No Homology to Known | | | |
| | | | | Proteins | | | |
| 359 | Gma.7487.2.A1_at | 0.403 | AW349599 | Transporter | | | Amino acid transporters |
| 360 | GmaAffx.7738.1.S1_s_at | 0.403 | BE058056 | No Homology to Known | | | |
| | | | | Proteins | | | |
| 361 | Gma.4124.1.S1_at | 0.403 | B1787263 | Transcription | Transcription | Homeodomain | |
| | | | | | Factor | | |
| 362 | GmaAffx.89141.1.S1_at | 0.403 | CK605765 | Cell Structure | | | O-methyltransferase |
| 363 | Gma.7051.1.A1_at | 0.403 | BU544I01 | Disease & Defense | | | Metacaspase involved in regulation of |
| | | | | | | | apoptosis |
| 364 | Gma.10854.1.S1_at | 0.403 | CA802334 | No Homology to Known | | | |
| | | | | Proteins | | | |
| 365 | GmaAffx.81502.1.S1_at | 0.403 | CD404946 | Unclassified - Hypothetical | | | Uncharacterized conserved protein |
| | | | | Protein NOT Supported by | | | |
| | | | | cDNA | | | |
| 366 | GmaAffx.53108.1.S1_at | 0.403 | BM093748 | Signal Transduction | Protein Kinase | | Serine/threonine protein kinase |
| 367 | Gma.15469.1.S1_at | 0.402 | BU546655 | Cell Structure | | | Alpha tubulin |
| 368 | GmaAffx.24809.1.S1_at | 0.402 | CA820582 | Unclassified - Hypothetical | | | |
| | | | | Protein NOT Supported by | | | |
| | | | | cDNA | | | |
| 369 | Gma.1114.1.S1_s_at | 0.402 | CD403139 | Unclassified - Hypothetical | | | |
| | | | | Protein NOT Supported by | | | |
| | | | | cDNA | | | |
| 370 | GmaAffx.92230.1.S1_at | 0.402 | CF807302 | Disease & Defense | | | |
| 371 | GmaAffx.10590.1.S1_s_at | 0.402 | BU551319 | Signal Transduction | Protein Kinase | | |
| 372 | GmaAffx.63822.1.S1_at | 0.402 | BM520838 | Disease & Defense | | | Purple (tartrate-resistant) acid |
| | | | | | | | phosphatase |
| 373 | GmaAffx.84607.2.S1_at | 0.401 | BU080867 | Unclassified - Hypothetical | | | |
| | | | | Protein Supported by cDNA | | | |
| 374 | GmaAffx.54085.1.S1_at | 0.401 | B1970762 | Unclassified - Hypothetical | | | |
| | | | | Protein Supported by cDNA | | | |
| 375 | GmaAffx.50347.1.S1_at | 0.4 | BQ740576 | Signal Transduction | Protein Kinase | | |
| 376 | GmaAffx.1694.1.S1_at | 0.4 | BQ454286 | Transcription | Transcription | MYB | |
| | | | | | Factor | | |
| 377 | Gma.1980.1.A1_at | 0.4 | BM520466 | Signal Transduction | Protein Kinase | AtCIPKs | Serine/threonine protein kinase |
| 378 | GmaAffx.60479.4.S1_at | 0.4 | BG507369 | Transcription | Transcription | Homeodomain | Transcription factor ME1S1 and related |
| | | | | | Factor | | HOX domain proteins |
| 379 | GmaAffx.15983.2.S1_at | 0.399 | AW279540 | Signal Transduction | Protein | | Serine/threonine protein phosphatase |
| | | | | | Phosphatase | | |
| 380 | GmaAffx.6824.2.S1_at | 0.399 | BQ094655 | Cell Structure | | | |
| 381 | Gma.3589.1.S1_at | 0.399 | AW101438 | Secondary Metabolism | GA Biosynthesis | | Iron/ascorbate family oxidoreductases |
| 382 | Gma.16585.1.S1_x_at | 0.399 | CA850597 | Metabolism | | | Predicted carbonic anhydrase involved |
| | | | | | | | in protection against oxidative damage |
| 383 | GmaAffx.57967.1.S1_at | 0.399 | AW102170 | Protein Destination & Storage | | | |
| 384 | Gma.5701.2.S1_at | 0.398 | B1701059 | Unclassified - Hypothetical | | | |
| | | | | Protein Supported by cDNA | | | |
| 385 | Gma.10885.1.S1_at | 0.398 | BQ610245 | Unclassified - Hypothetical | | | Predicted kinase |
| | | | | Protein Supported by cDNA | | | |
| 386 | Gma.11665.2.A1_at | 0.397 | BU545959 | No Homology to Known | | | |
| | | | | Proteins | | | |
| 387 | Gma.10626.3.S1_at | 0.397 | BE022458 | Secondary Metabolism | | | Iron/ascorbate family oxidoreductases |
| 388 | Gma.5038.1.A1_at | 0.397 | BE023631 | Disease & Defense | | | Acyl-CoA synthetase |
| 389 | GmaAffx.43786.2.S1_at | 0.396 | BU080930 | Signal Transduction | Histidine Kinase | | Sensory transduction histidine kinase |
| 390 | GmaAffx.24844.1.S1_at | 0.396 | BU765373 | Unclassified - Hypothetical | | | Uncharacterized conserved protein |
| | | | | Protein Supported by cDNA | | | |
| 391 | GmaAffx.27787.1.A1_at | 0.396 | BG789758 | Transporter | | | Transporter, ABC superfamily (Breast |
| | | | | | | | cancer resistance protein) |
| 392 | Gma.1380.1.S1_at | 0.396 | CA935076 | Metabolism | | | |
| 393 | Gma.4260.1.S1_at | 0.396 | BM177256 | Unclassified - Hypothetical | | | |
| | | | | Protein NOT Supported by | | | |
| | | | | cDNA | | | |
| 394 | GmaAffx.74693.1.S1_at | 0.396 | CD406171 | Metabolism | | | |
| 395 | Gma.5709.1.S1_at | 0.395 | BG157556 | Unclassified - Protein with | | | |
| | | | | Unknown Function | | | |
| 396 | GmaAffx.22297.2.S1_at | 0.395 | BM271210 | Transcription | Transcription | WRKY | |
| | | | | | Factor | | |
| 397 | Gma.5578.2.S1_a_at | 0.394 | AW310579 | Metabolism | Cytochrorne | CYP98A | Cytochrorne P450 CYP2 subfamily |
| | | | | | P450 | | |
| 398 | GmaAffx.74160.1.S1_at | 0.394 | BQ740845 | Protein Destination & Storage | | | |
| 399 | Gma.177.1.S1_at | 0.393 | AF004806.1 | Protein Destination & Storage | Storage Protein | | Reticulon |
| 400 | GmaAffx.13730.1.S1_at | 0.393 | B1316755 | Transcription | Transcription | Basic Helix-Loop- | |
| | | | | | Factor | Helix (bHLH) | |
| 401 | GmaAffx.28870.1.S1_at | 0.393 | BG726532 | Unclassified - Hypothetical | | | |
| | | | | Protein Supported by cDNA | | | |
| 402 | GmaAffx.73195.1.S1_at | 0.393 | B1701045 | Secondary Metabolism | Flavonoid | | Flavonol reductase/cinnamoyl-CoA |
| | | | | | Biosynthesis | | reductase |
| 403 | Gma.16147.1.S1_at | 0.393 | BE473577 | Transcription | Transcription | Basic Helix-Loop- | |
| | | | | | Factor | Helix (bHLH) | |
| 404 | GmaAffx.23789.1.S1_at | 0.392 | BU551014 | Unclassified - Hypothetical | | | |
| | | | | Protein Supported by cDNA | | | |
| 405 | GmaAffx.37636.1.S1_at | 0.392 | BQ610347 | Metabolism | Amino Acid | | Alanine-glyoxylate aminotransferase |
| | | | | | Biosynthesis/ | | AGT2 |
| | | | | | Metabolism | | |
| 406 | Gma.7047.1.S1_s_at | 0.392 | CD417401 | Unclassified - Protein with | | | |
| | | | | Unknown Function | | | |
| 407 | Gma.1649.1.A1_at | 0.392 | BE659535 | No Homology to Known | | | |
| | | | | Proteins | | | |
| 408 | GmaAffx.8304.1.A1_at | 0.391 | BE822695 | Unclassified - Hypothetical | | | Possible oxidoreductase |
| | | | | Protein Supported by cDNA | | | |
| 409 | GmaAffx.3726.1.A1_at | 0.391 | BE822942 | Unclassified - Hypothetical | | | |
| | | | | Protein Supported by cDNA | | | |
| 410 | Gma.5215.1.S1_at | 0.391 | AI988141 | Unclassified - Hypothetical | | | BCL2-associated athanogene-like |
| | | | | Protein Supported by cDNA | | | proteins and related BAG family |
| | | | | | | | chaperone regulators |
| 411 | Gma.17605.1.S1_at | 0.39 | BI968208 | Secondary Metabolism | Flavonoid | | |
| | | | | | Biosynthesis | | |
| 412 | GmaAffx.58125.1.A1_at | 0.39 | BE347561 | Transcription | Transcription | Basic Helix-Loop- | |
| | | | | | Factor | Helix (bHLH) | |
| 413 | GmaAffx.89624.1.A1_s_at | 0.39 | CK606100 | Transcription | Transcription | Basic Leucine | |
| | | | | | Factor | Zipper (bZIP) | |
| 414 | Gma.3745.1.S1_at | 0.389 | AW156339 | Secondary Metabolism | Amines | | Amine oxidase |
| | | | | | Biosynthesis | | |
| 415 | GmaAffx.82034.1.S1_at | 0.389 | BM887093 | Transcription | Transcription | Homeodomain | Transcription factor HEX, contains |
| | | | | | Factor | | HOX and HALZ domains |
| 416 | Gma.16336.1.S1_at | 0.389 | BE659555 | Protein Destination & Storage | | | Predicted pilin-like transcription factor |
| 417 | Gma.16276.2.S1_a_at | 0.388 | BG316414 | Unclassified - Hypothetical | | | FOG: PPR repeat |
| | | | | Protein Supported by cDNA | | | |
| 418 | Gma.13146.1.S1_at | 0.388 | BM731716 | Unclassified - Hypothetical | | | |
| | | | | Protein Supported by cDNA | | | |
| 419 | GmaAffx.86020.1.S1_at | 0.388 | B1701699 | Signal Transduction | Protein Kinase | AtCIPKs | Serine/threonine protein kinase |
| 420 | Gma.3591.1.S1_a_at | 0.388 | BE440266 | Secondary Metabolism | Ethylene | | Iron/ascorbate family oxidoreductases |
| | | | | | Biosynthesis | | |
| 421 | GmaAffx.68853.1.S1_at | 0.388 | CA803108 | Unclassified - Hypothetical | | | |
| | | | | Protein Supported by cDNA | | | |
| 422 | GmaAffx.73021.1.S1_at | 0.387 | BE556340 | Disease & Defense | | | |
| 423 | GmaAffx.80899.1.S1_at | 0.387 | AW348263 | Unclassified - Hypothetical | | | |
| | | | | Protein Supported by cDNA | | | |
| 424 | GmaAffx.77052.1.S1_at | 0.387 | BE556140 | Transporter | Inorganic Solute | Putative | |
| | | | | | Cotransporters | potassium | |
| | | | | | | transporter family | |
| 425 | GmaAffx.91731.1.S1_s_at | 0.387 | CF807456 | Secondary Metabolism | Ethylene | | Iron/ascorbate family oxidoreductases |
| | | | | | Biosynthesis | | |
| 426 | GmaAffx.77052.2.A1_at | 0.387 | BG155285 | Transporter | Inorganic Solute | Putative | |
| | | | | | Cotransporters | potassium | |
| | | | | | | transporter family | |
| 427 | GmaAffx.17924.1.S1_at | 0.387 | BG157338 | Unclassified - Protein with | | | |
| | | | | Unknown Function | | | |
| 428 | GmaAffx.59014.1.S1_s_at | 0.386 | AW733977 | No Homology to Known | | | |
| | | | | Proteins | | | |
| 429 | Gma.8946.1.S1_at | 0.386 | BQ453813 | Transcription | Transcription | NAC Domain | |
| | | | | | Factor | | |
| 430 | Gma.2475.1.S1_at | 0.384 | BM522941 | Cell Structure | | | |
| 431 | Gma.6730.1.S1_at | 0.384 | BE211440 | Metabolism | | | UDP-glucuronosyl and UDP-glucosyl |
| | | | | | | | transferase |
| 432 | GmaAffx.13912.1.S1_at | 0.384 | B1699258 | Disease & Defense | | | Purple (tartrate-resistant) acid |
| | | | | | | | phosphatase |
| 433 | Gma.5038.2.S1_a_at | 0.384 | CD417606 | Disease & Defense | | | Acyl-CoA synthetase |
| 434 | Gma.12817.1.A1_at | 0.384 | CD405335 | Unclassified - Hypothetical | | | |
| | | | | Protein Supported by cDNA | | | |
| 435 | GmaAffx.31571.1.S1_at | 0.384 | BU551140 | Unclassified - Hypothetical | | | |
| | | | | Protein NOT Supported by | | | |
| | | | | cDNA | | | |
| 436 | Gma.4990.1.A1_at | 0.383 | B1315863 | Unclassified - Hypothetical | | | Uncharacterized conserved protein |
| | | | | Protein Supported by cDNA | | | |
| 437 | Gma.13340.1.S1_at | 0.382 | BM523855 | Metabolism | | | UDP-glucuronosyl and UDP-glucosyl |
| | | | | | | | transferase |
| 438 | GmaAffx.28196.2.A1_s_at | 0.382 | CD416781 | Metabolism | | | Long-chain acyl-CoA synthetases (AMP- |
| | | | | | | | forming) |
| 439 | GmaAffx.90585.1.S1_s_at | 0.382 | CF806310 | Protein Destination & Storage | | | Aspartyl protease |
| 440 | GmaAffx.62856.1.S1_at | 0.382 | AW432978 | Signal Transduction | MAPKKK | ZlK | Serine/threonine protein kinase |
| 441 | GmaAffx.6.1.S1_at | 0.381 | AW348562 | No Homology to Known | | | |
| | | | | Proteins | | | |
| 442 | Gma.2565.1.S1_at | 0.381 | BG652533 | Unclassified - Hypothetical | | | |
| | | | | Protein Supported by cDNA | | | |
| 443 | Gma.8706.1.S1_at | 0.38 | BU762562 | Unclassified - Hypothetical | | | |
| | | | | Protein Supported by cDNA | | | |
| 444 | GmaAffx.39467.1.S1_at | 0.38 | BQ299569 | Unclassified - Hypothetical | | | |
| | | | | Protein NOT Supported by | | | |
| | | | | cDNA | | | |
| 445 | GmaAffx.90789.1.S1_s_at | 0.38 | CF806514 | Unclassified - Protein with | | | |
| | | | | Unknown Function | | | |
| 446 | Gma.9828.1.S1_at | 0.38 | AF529301.1 | Metabolism | | | Haloacid dehalogenase-like hydrolase |
| 447 | GmaAffx.93642.1.S1_s_at | 0.379 | CF809367 | Disease & Defense | | | |
| 448 | GmaAffx.6790.1.S1_at | 0.379 | AI856683 | Disease & Defense | | | Soluble epoxide hydrolase |
| 449 | Gma.4049.1.S1_at | 0.379 | BQ786519 | Signal Transduction | Protein Kinase | | Serine/threonine protein kinase |
| 450 | GmaAffx.38961.1.S1_at | 0.379 | BU081740 | Transcription | Transcription | MYB | Transcription factor, Myb superfamily |
| | | | | | Factor | | |
| 451 | Gma.5691.1.S1_at | 0.379 | BM522643 | No Homology to Known | | | |
| | | | | Proteins | | | |
| 452 | Gma.5578.3.S1_at | 0.379 | AW310572 | Metabolism | Cytochrome | CYP98A | Cytochrome P450 CYP2 subfamily |
| | | | | | P450 | | |
| 453 | GmaAffx.85878.1.S1_at | 0.378 | BF067810 | Intracellular Traffic | | | Aquaporin (major intrinsic protein |
| | | | | | | | family) |
| 454 | GmaAffx.42856.1.S1_at | 0.378 | BM567981 | Transcription | Transcription | Basic Helix-Loop- | |
| | | | | | Factor | Helix (bHLH) | |
| 455 | Gma.6343.1.S1_at | 0.377 | AI442963 | Unclassified - Hypothetical | | | |
| | | | | Protein NOT Supported by | | | |
| | | | | cDNA | | | |
| 456 | GmaAffx.61571.2.S1_at | 0.377 | AI939190 | Unclassified - Hypothetical | | | |
| | | | | Protein NOT Supported by | | | |
| | | | | cDNA | | | |
| 457 | GmaAffx.59808.1.S1_at | 0.377 | BM886228 | Transporter | | | Predicted transporter (major facilitator |
| | | | | | | | superfamily) |
| 458 | Gma.2831.1.S1_s_at | 0.376 | AW349413 | Transcription | Transcription | Zinc Finger | |
| | | | | | Factor | | |
| 459 | GmaAffx.40923.1.S1_at | 0.376 | BG653848 | Disease & Defense | | | Selenium-binding protein |
| 460 | Gma.559.1.A1_at | 0.376 | BQ630327 | Protein Destination & Storage | | | |
| 461 | GmaAffx.24451.1.A1_at | 0.375 | BU550405 | Metabolism | Cytochrome | CYP90C | Cytochrome P450 |
| | | | | | P450 | | CYP4/CYP19/CYP26 subfamilies |
| 462 | GmaAffx.38558.1.S1_at | 0.375 | CD407094 | Metabolism | | | NADP+-dependent malic enzyme |
| 463 | GmaAffx.17892.2.S1_at | 0.375 | BQ628677 | No Homology to Known | | | |
| | | | | Proteins | | | |
| 464 | GmaAffx.2745.2.A1_at | 0.374 | BQ611985 | Unclassified - Hypothetical | | | Acetylglucosaminyltransferase |
| | | | | Protein Supported by cDNA | | | EXT1/exostosin 1 |
| 465 | GmaAffx.91254.1.S1_at | 0.374 | CF806979 | Unclassified - Hypothetical | | | |
| | | | | Protein Supported by cDNA | | | |
| 466 | GmaAffx.46770.1.S1_at | 0.374 | BQ298902 | Transporter | lon Channel | Putative ligand- | Glutamate-gated kainate-type ion |
| | | | | | Families | gated ion channel | channel receptor subunit GluR5 and |
| | | | | | | subunit family | related subunits |
| 467 | GmaAffx.1620.1.S1_at | 0.374 | AW424105 | Unclassified - Hypothetical | | | |
| | | | | Protein NOT Supported by | | | |
| | | | | cDNA | | | |
| 468 | GmaAffx.90570.1.S1_at | 0.374 | CF806295 | Unclassified - Hypothetical | | | |
| | | | | Protein Supported by cDNA | | | |
| 469 | GmaAffx.84964.1.S1_at | 0.373 | AW598267 | No Homology to Known | | | |
| | | | | Proteins | | | |
| 470 | GmaAffx.64644.1.S1_at | 0.373 | AW348952 | Signal Transduction | Protein Kinase | | Serine/threonine protein kinase |
| 471 | GmaAffx.79121.1.S1_at | 0.373 | CA801737 | Transcription | Transcription | Zinc Finger | CCCH-type Zn-finger protein |
| | | | | | Factor | | |
| 472 | Gma.5578.3.S1_x_at | 0.372 | AW310572 | Metabolism | Cytochrome | CYP98A | Cytochrome P450 CYP2 subfamily |
| | | | | | P450 | | |
| 473 | GmaAffx.32011.1.S1_at | 0.372 | BI701198 | No Homology to Known | | | |
| | | | | Proteins | | | |
| 474 | Gma.8240.1.S1_at | 0.371 | BM092761 | Secondary Metabolism | | | Iron/ascorbate family oxidoreductases |
| 475 | GmaAffx.35729.1.S1_s_at | 0.37 | BE806047 | Unclassified - Hypothetical | | | Predicted E3 ubiquitin ligase |
| | | | | Protein Supported by cDNA | | | |
| 476 | GmaAffx.92462.1.S1_at | 0.37 | CF808187 | Transcription | Transcription | Zinc Finger | FOG: Predicted E3 ubiquitin ligase |
| | | | | | Factor | | |
| 477 | Gma.10615.1.A1_at | 0.37 | BU545626 | Transcription | Transcription | Basic Helix-Loop- | |
| | | | | | Factor | Helix (bHLH) | |
| 478 | GmaAffx.61272.1.S1_at | 0.369 | B1972394 | No Homology to Known | | | |
| | | | | Proteins | | | |
| 479 | GmaAffx.6008.1.S1_at | 0.369 | BU550119 | Transcription | Transcription | Basic Helix-Loop- | |
| | | | | | Factor | Helix (bHLH) | |
| 480 | GmaAffx.44716.1.A1_at | 0.368 | B1969342 | Disease & Defense | | | |
| 481 | GmaAffx.2745.1.S1_at | 0.368 | BF009213 | Unclassified - Hypothetical | | | Acetylglucosaminyltransferase |
| | | | | Protein Supported by cDNA | | | EXT1/exostosin 1 |
| 482 | Gma.5104.1.S1_at | 0.368 | BE611202 | Unclassified - Hypothetical | | | |
| | | | | Protein Supported by cDNA | | | |
| 483 | Gma.1332.1.S1_x_at | 0.368 | B1970666 | Secondary Metabolism | Ethylene | | 1-aminocyclopropane-1-carboxylate |
| | | | | | Biosynthesis | | synthase, and related proteins |
| 484 | Gma.10331.1.A1_at | 0.368 | CD402553 | Unclassified - Protein with | | | Macrophage migration inhibitory factor |
| | | | | Unknown Function | | | |
| 485 | GmaAffx.91917.1.S1_s_at | 0.368 | CF807642 | Unclassified - Hypothetical | | | |
| | | | | Protein Supported by cDNA | | | |
| 486 | GmaAffx.4445.1.A1_at | 0.368 | BU547894 | Transcription | Transcription | AP2/EREBP | |
| | | | | | Factor | | |
| 487 | GmaAffx.92537.1.S1_at | 0.367 | CF808262 | Energy | | | Thioredoxin |
| 488 | GmaAffx.44471.1.A1_at | 0.367 | CA851833 | Unclassified - Hypothetical | | | |
| | | | | Protein NOT Supported by | | | |
| | | | | cDNA | | | |
| 489 | GmaAffx.86613.2.S1_at | 0.367 | BE024005 | Metabolism | | | Glutaredoxin and related proteins |
| 490 | Gma.1263.1.S1_at | 0.366 | AY144180.1 | Metabolism | | | Ca2+/calmodulin-dependent protein |
| | | | | | | | kinase, EF-Hand protein superfamily |
| 491 | GmaAffx.87493.1.S1_at | 0.366 | AW781752 | Unclassified - Hypothetical | | | |
| | | | | Protein NOT Supported by | | | |
| | | | | cDNA | | | |
| 492 | GmaAffx.7771.1.S1_at | 0.366 | BE022913 | Transporter | Inorganic Solute | Putative sulfate | Sulfate/bicarbonate/oxalate exchanger |
| | | | | | Cotransporters | transporter family | SAT-1 and related transporters (SLC26 |
| | | | | | | | family) |
| 493 | GmaAffx.93214.1.S1_at | 0.366 | CF808939 | No Homology to Known | | | |
| | | | | Proteins | | | |
| 494 | GmaAffx.36972.1.S1_at | 0.366 | BQ079450 | Unclassified - Hypothetical | | | |
| | | | | Protein NOT Supported by | | | |
| | | | | cDNA | | | |
| 495 | Gma.1979.1.S1_a_at | 0.366 | CD403280 | Unclassified - Protein with | | | Multitransmembrane protein |
| | | | | Unknown Function | | | |
| 496 | Gma.8160.1.S1_at | 0.366 | B1787214 | Unclassified - Hypothetical | | | |
| | | | | Protein NOT Supported by | | | |
| | | | | cDNA | | | |
| 497 | GmaAffx.4936.1.S1_at | 0.365 | AW666270 | Transporter | | | Multidrug/pheromone exporter, ABC |
| | | | | | | | superfamily |
| 498 | GmaAffx.23475.1.A1_at | 0.365 | BU548049 | Unclassified - Hypothetical | | | |
| | | | | Protein Supported by cDNA | | | |
| 499 | GmaAffx.68060.2.A1_at | 0.365 | AW396457 | No Homology to Known | | | |
| | | | | Proteins | | | |
| 500 | Gma.3511.4.S1_at | 0.364 | BM526875 | Cell Structure | | | |
| 501 | Gma.13013.2.S1_a_at | 0.364 | AW457857 | Unclassified - Hypothetical | | | |
| | | | | Protein Supported by cDNA | | | |
| 502 | GmaAffx.81851.1.S1_at | 0.364 | AW706751 | Unclassified - Hypothetical | | | |
| | | | | Protein Supported by cDNA | | | |
| 503 | GmaAffx.81399.1.S1_at | 0.364 | AW568772 | Cell Growth & Division | | | |
| 504 | Gma.2342.1.S1_at | 0.363 | Z46953.1 | Transcription | Transcription | Heat Shock | Heat shock transcription factor |
| | | | | | Factor | | |
| 505 | GmaAffx.74390.1.A1_at | 0.363 | BU544169 | Protein Destination & Storage | | | Molecular chaperone (DnaJ superfamily) |
| 506 | Gma.6836.1.A1_at | 0.363 | AW349I21 | Unclassified - Hypothetical | | | Predicted pyroglutamyl peptidase |
| | | | | Protein Supported by cDNA | | | |
| 507 | GmaAffx.37477.1.S1_s_at | 0.363 | BQ081355 | Metabolism | | | Branched chain aminotransferase |
| | | | | | | | BCAT1, pyridoxal phosphate enzymes |
| | | | | | | | type IV superfamily |
| 508 | GmaAffx.75740.1.S1_at | 0.363 | BG157373 | Unclassified - Hypothetical | | | |
| | | | | Protein NOT Supported by | | | |
| | | | | cDNA | | | |
| 509 | GmaAffx.48042.1.S1_at | 0.362 | AI960210 | Transporter | | | Aquaporin (major intrinsic protein |
| | | | | | | | family) |
| 510 | GmaAffx.23295.1.S1_at | 0.362 | BI968028 | Disease & Defense | | | |
| 511 | GmaAffx.17892.1.S1_at | 0.362 | AI442822 | Unclassified - Hypothetical | | | |
| | | | | Protein Supported by cDNA | | | |
| 512 | Gma.5309.1.S1_at | 0.362 | BM092263 | Unclassified - Hypothetical | | | |
| | | | | Protein NOT Supported by | | | |
| | | | | cDNA | | | |
| 513 | GmaAffx.25598.1.S1_at | 0.362 | AW311255 | No Homology to Known | | | |
| | | | | Proteins | | | |
| 514 | GmaAffx.84261.1.S1_at | 0.362 | BI701092 | Transcription | Transcription | AP2/EREBP | |
| | | | | | Factor | | |
| 515 | Gma.14075.1.S1_at | 0.362 | CD415333 | Protein Destination & Storage | Storage Protein | | Ca2+-independent phospholipase A2 |
| 516 | GmaAffx.82582.1.S1_at | 0.361 | BM139949 | No Homology to Known | | | |
| | | | | Proteins | | | |
| 517 | GmaAffx.93342.1.S1_s_at | 0.361 | CF809067 | Disease & Defense | | | Glutathione peroxidase |
| 518 | Gma.11269.1.S1_at | 0.36 | CD395046 | Transporter | | | |
| 519 | GmaAffx.83715.1.S1_at | 0.36 | BI785710 | Transporter | | | Glutaredoxin-related protein |
| 520 | Gma.1188.2.S1_at | 0.36 | BG406072 | Cell Growth & Division | | | |
| 521 | Gma.7227.2.S1_a_at | 0.36 | AW759183 | Protein Destination & Storage | | | Ubiquitin-protein ligase |
| 522 | GmaAffx.92127.1.S1_s_at | 0.359 | CF807852 | Transcription | Transcription | AP2/EREBP | |
| | | | | | Factor | | |
| 523 | GmaAffx.28397.1.S1_at | 0.359 | BM568229 | Metabolism | | | |
| 524 | Gma.364.1.A1_at | 0.359 | AW309875 | Unclassified - Hypothetical | | | |
| | | | | Protein Supported by cDNA | | | |
| 525 | GmaAffx.86675.1.S1_at | 0.359 | B1424294 | No Homology to Known | | | |
| | | | | Proteins | | | |
| 526 | Gma.5331.1.S1_a_at | 0.359 | BI892702 | Transcription | Transcription | NAC Domain | |
| | | | | | Factor | | |
| 527 | GmaAffx.5285.1.S1_at | 0.359 | BM523736 | Metabolism | | | |
| 528 | Gma.6213.1.S1_at | 0.358 | CD401652 | Protein Destination & Storage | | | Glutathione S-transferase |
| 529 | GmaAffx.84607.1.S1_at | 0.357 | BF596776 | Unclassified - Hypothetical | | | |
| | | | | Protein Supported by cDNA | | | |
| 530 | GmaAffx.11857.1.A1_at | 0.357 | AW351021 | Unclassified - Hypothetical | | | |
| | | | | Protein Supported by cDNA | | | |
| 531 | Gma.17007.1.S1_at | 0.357 | BE820533 | No Homology to Known | | | |
| | | | | Proteins | | | |
| 532 | GmaAffx.37615.1.S1_at | 0.357 | BQ454150 | No Homology to Known | | | |
| | | | | Proteins | | | |
| 533 | GmaAffx.15471.2.S1_at | 0.357 | B1784829 | Transcription | Transcription | MYB | Transcription factor, Myb superfamily |
| | | | | | Factor | | |
| 534 | Gma.5294.1.S1_at | 0.357 | BE658252 | Unclassified - Hypothetical | | | |
| | | | | Protein Supported by cDNA | | | |
| 535 | GmaAffx.43774.1.A1_at | 0.357 | AW349173 | Cell Structure | | | |
| 536 | GmaAffx.74933.1.S1_at | 0.355 | CA935084 | Unclassified - Protein with | | | Aspartyl protease |
| | | | | Unknown Function | | | |
| 537 | GmaAffx.37458.2.S1_at | 0.355 | BQ079820 | Unclassified - Hypothetical | | | |
| | | | | Protein NOT Supported by | | | |
| | | | | cDNA | | | |
| 538 | Gma.2602.1.S1_at | 0.355 | BU577743 | No Homology to Known | | | |
| | | | | Proteins | | | |
| 539 | Gma.4336.1.S1_at | 0.355 | BQ079907 | Unclassified - Hypothetical | | | |
| | | | | Protein NOT Supported by | | | |
| | | | | cDNA | | | |
| 540 | Gma.5478.1.S1_at | 0.353 | AW350606 | Metabolism | | | |
| 541 | Gma.10701.1.S1_at | 0.353 | BF596612 | Unclassified - Hypothetical | | | |
| | | | | Protein Supported by cDNA | | | |
| 542 | GmaAffx.37055.1.A1_at | 0.353 | BM142939 | Metabolism | | | UDP-glucuronosyl and UDP-glucosyl |
| | | | | | | | transferase |
| 543 | Gma.11119.2.S1_s_at | 0.353 | CF920743 | Disease & Defense | | | |
| 544 | GmaAffx.13016.1.S1_at | 0.352 | BF069324 | No Homology to Known | | | |
| | | | | Proteins | | | |
| 545 | Gma.8331.1.S1_at | 0.352 | BE659403 | Metabolism | | | Aldehyde dehydrogenase |
| 546 | Gma.11277.1.S1_at | 0.351 | CA802310 | Protein Destination & Storage | | | |
| 547 | Gma.10365.1.S1_at | 0.351 | BG725377 | Unclassified - Hypothetical | | | |
| | | | | Protein Supported by cDNA | | | |
| 548 | GmaAffx.70210.1.S1_at | 0.351 | B1699931 | Transcription | Transcription | Unclassified | |
| | | | | | Factor | | |
| 549 | Gma.14567.1.S1_at | 0.35 | BE824123 | Disease & Defense | | | |
| 550 | Gma.14325.1.S1_at | 0.35 | B1700686 | Disease & Defense | | | Ca2+-independent phospholipase A2 |
| 551 | Gma.4372.1.S1_at | 0.35 | AI443505 | Secondary Metabolism | | | Reductases with broad range of substrate |
| | | | | | | | specificities |
| 552 | GmaAffx.66676.1.S1_at | 0.35 | BQ610288 | Unclassified - Protein with | | | |
| | | | | Unknown Function | | | |
| 553 | Gma.13418.1.S1_at | 0.35 | BM143894 | Transporter | | | |
| 554 | Gma.17688.1.S1_at | 0.349 | CA937360 | Transcription | Transcription | Basic Helix-Loop- | |
| | | | | | Factor | Helix (bHLH) | |
| 555 | GmaAffx.92166.1.S1_s_at | 0.349 | CF806313 | Signal Transduction | Unclassified | | Aspartyl protease |
| 556 | GmaAffx.37574.1.S1_s_at | 0.349 | BQ298504 | Secondary Metabolism | | | Hydroxyindole-O-methyltransferase and |
| | | | | | | | related SAM-dependent |
| | | | | | | | methyltransferases |
| 557 | Gma.382.1.A1_at | 0.349 | AW349068 | Metabolism | | | Arylacetamide deacetylase |
| 558 | Gma.752.1.A1_at | 0.349 | AW432463 | Transcription | Transcription | Basic Helix-Loop- | |
| | | | | | Factor | Helix (bHLH) | |
| 559 | GmaAffx.32765.1.S1_at | 0.348 | BE024072 | No Homology to Known | | | |
| | | | | Proteins | | | |
| 560 | Gma.968.1.S1_at | 0.348 | D28876.1 | Protein Destination & Storage | | | Asparaginyl peptidases |
| 561 | Gma.10756.1.S1_s_at | 0.348 | BE822092 | Unclassified - Hypothetical | | | |
| | | | | Protein Supported by cDNA | | | |
| 562 | GmaAffx.23276.1.S1_at | 0.347 | AI856198 | Transporter | | | Amino acid transporters |
| 563 | GmaAffx.50673.2.S1_s_at | 0.347 | B1787147 | Transcription | Transcription | NAC Domain | |
| | | | | | Factor | | |
| 564 | GmaAffx.24359.1.A1_at | 0.346 | BU544840 | Metabolism | | | |
| 565 | GmaAffx.57469.1.S1_at | 0.346 | AW351054 | Unclassified - Hypothetical | | | |
| | | | | Protein Supported by cDNA | | | |
| 566 | GmaAffx.91561.1.S1_s_at | 0.346 | CF807286 | Transporter | | | Apolipoprotein D/Lipocalin |
| 567 | Gma.5084.1.S1_at | 0.346 | AW309848 | Protein Destination & Storage | Storage Protein | | |
| 568 | Gma.3888.2.S1_at | 0.345 | BM139532 | Unclassified - Hypothetical | | | Aldehyde reductase |
| | | | | Protein NOT Supported by | | | |
| | | | | cDNA | | | |
| 569 | Gma.16359.1.A1_at | 0.344 | B1970359 | Metabolism | | | GPI-alpha-mannosyltransferase III |
| | | | | | | | (GPII0/PIG-B) involved in |
| | | | | | | | glycosylphosphatidylinositol anchor |
| | | | | | | | biosynthesis |
| 570 | Gma.8530.1.S1_at | 0.344 | AF109720.1 | Protein Destination & Storage | Storage Protein | | |
| 571 | Gma.9056.1.S1_at | 0.343 | CA819787 | Metabolism | | | Acyl-CoA-binding protein |
| 572 | GmaAffx.70056.1.S1_at | 0.343 | BU765705 | No Homology to Known | | | |
| | | | | Proteins | | | |
| 573 | Gma.106I2.I.S1_s_at | 0.343 | BG650314 | No Homology to Known | | | |
| | | | | Proteins | | | |
| 574 | GmaAffx.1301.52.S1_at | 0.343 | BG510366 | Unclassified - Hypothetical | | | |
| | | | | Protein Supported by cDNA | | | |
| 575 | GmaAffx.65217.1.A1_at | 0.343 | BU543988 | Metabolism | | | Acyl-CoA-binding protein |
| 576 | Gma.17729.1.S1_s_at | 0.343 | CD393936 | No Homology to Known | | | |
| | | | | Proteins | | | |
| 577 | GmaAffx.15983.2.S1_s_at | 0.343 | AW279540 | Signal Transduction | Protein | | Serine/threonine protein phosphatase |
| | | | | | Phosphatase | | |
| 578 | GmaAffx.83964.1.S1_at | 0.342 | BU082747 | Unclassified - Protein with | | | Protein involved in mRNA turnover and |
| | | | | Unknown Function | | | stability |
| 579 | GmaAffx.33408.1.S1_at | 0.342 | BU547044 | No Homology to Known | | | |
| | | | | Proteins | | | |
| 580 | Gma.1137.1.S1_x_at | 0.342 | BF067710 | Metabolism | | | |
| 581 | GmaAffx.46681.1.S1_at | 0.342 | BQ080794 | Unclassified - Hypothetical | | | |
| | | | | Protein Supported by cDNA | | | |
| 582 | Gma.5256.1.A1_at | 0.341 | CD408042 | No Homology to Known | | | |
| | | | | Proteins | | | |
| 583 | Gma.11277.1.S1_x_at | 0.341 | CA802310 | Protein Destination & Storage | | | |
| 584 | Gma.2952.1.S1_at | 0.341 | BM270250 | No Homology to Known | | | |
| | | | | Proteins | | | |
| 585 | Gma.13035.1.S1_at | 0.341 | CD396536 | No Homology to Known | | | |
| | | | | Proteins | | | |
| 586 | Gma.15863.1.S1_s_at | 0.34 | CD413163 | Metabolism | | | |
| 587 | Gma.5255.1.S1_at | 0.34 | AI736533 | Unclassified - Hypothetical | | | |
| | | | | Protein Supported by cDNA | | | |
| 588 | Gma.12902.1.A1_at | 0.34 | CD395017 | Metabolism | | | Hydroxyindole-O-methyltransferase and |
| | | | | | | | related SAM-dependent |
| | | | | | | | methyltransferases |
| 589 | Gma.3917.1.S1_at | 0.34 | BE658782 | Cell Structure | | | Pectin acetylesterase and similar |
| | | | | | | | proteins |
| 590 | Gma.9907.1.S1_at | 0.339 | AW309518 | Transcription | Transcription | Basic Helix-Loop- | |
| | | | | | Factor | Helix (bHLH) | |
| 591 | GmaAffx.29254.1.S1_at | 0.338 | AW760361 | Transcription | Transcription | Basic Helix-Loop- | |
| | | | | | Factor | Helix (bHLH) | |
| 592 | GmaAffx.31862.1.S1_at | 0.338 | BE822501 | Unclassified - Hypothetical | | | |
| | | | | Protein Supported by cDNA | | | |
| 593 | GmaAffx.37930.1.A1_at | 0.338 | BU547709 | Cell Structure | | | |
| 594 | Gma.5578.4.S1_a_at | 0.338 | BE023932 | Metabolism | Cytochrome | CYP98A | Cytochrome P450 CYP2 subfamily |
| | | | | | P450 | | |
| 595 | GmaAffx.37458.1.S1_at | 0.338 | BU549419 | Unclassified - Protein with | | | |
| | | | | Unknown Function | | | |
| 596 | Gma.10824.1.S1_at | 0.338 | BE824257 | Unclassified - Hypothetical | | | Magnesium transporters: CorA family |
| | | | | Protein Supported by cDNA | | | |
| 597 | GmaAffx.93189.1.S1_at | 0.338 | CF808914 | Metabolism | | | Carbon-nitrogen hydrolase |
| 598 | Gma.4222.1.S1_at | 0.337 | BU083971 | Unclassified - Hypothetical | | | Arylacetamide deacetylase |
| | | | | Protein NOT Supported by | | | |
| | | | | cDNA | | | |
| 599 | GmaAffx.64075.1.S1_at | 0.337 | B1700868 | Unclassified - Hypothetical | | | |
| | | | | Protein Supported by cDNA | | | |
| 600 | GmaAffx.88945.1.S1_at | 0.337 | CK605738 | Unclassified - Protein with | | | |
| | | | | Unknown Function | | | |
| 601 | GmaAffx.91328.1.S1_at | 0.336 | CF807053 | Unclassified - Hypothetical | | | Leucine rich repeat proteins, some |
| | | | | Protein Supported by cDNA | | | proteins contain F-box |
| 602 | Gma.10827.2.S1_s_at | 0.335 | BQ612452 | Protein Destination & Storage | Storage Protein | | |
| 603 | GmaAffx.92070.1.S1_at | 0.335 | CF807049 | Cell Structure | | | O-methyltransferase |
| 604 | GmaAffx.10908.1.S1_at | 0.335 | BE822295 | Transcription | Transcription | Homeodomain | |
| | | | | | Factor | | |
| 605 | Gma.17610.1.S1_x_at | 0.334 | U50075.1 | Metabolism | | | |
| 606 | Gma.14361.1.A1_at | 0.333 | CD409843 | No Homology to Known | | | |
| | | | | Proteins | | | |
| 607 | Gma.12731.2.A1_s_at | 0.333 | CD395848 | Unclassified - Hypothetical | | | Aquaporin (major intrinsic protein |
| | | | | Protein NOT Supported by | | | family) |
| | | | | cDNA | | | |
| 608 | GmaAffx.93183.1.S1_s_at | 0.333 | CF808908 | Secondary Metabolism | | | S-adenosylmethionine decarboxylase |
| 609 | GmaAffx.46103.1.S1_at | 0.333 | BM091933 | Metabolism | | | Hydroxymethylglutaryl-CoA lyase |
| 610 | Gma.336.1.A1_at | 0.333 | AW310815 | Metabolism | Sulfur | | |
| | | | | | Metabolism | | |
| 611 | GmaAffx.41990.1.S1_at | 0.332 | CA936708 | No Homology to Known | | | |
| | | | | Proteins | | | |
| 612 | Gma.6782.2.S1_a_at | 0.332 | AW156813 | Unclassified - Hypothetical | | | |
| | | | | Protein Supported by cDNA | | | |
| 613 | GmaAffx.93182.1.S1_s_at | 0.332 | CF808907 | Secondary Metabolism | Ethylene | | Iron/ascorbate family oxidoreductases |
| | | | | | Biosynthesis | | |
| 614 | GmaAffx.62364.1.S1_at | 0.331 | AW733764 | Protein Destination & Storage | | | Dipeptidyl aminopeptidase |
| 615 | Gma.8312.1.A1_at | 0.331 | AW310254 | Unclassified - Protein with | | | |
| | | | | Unknown Function | | | |
| 616 | GmaAffx.86844.1.S1_at | 0.331 | BE822146 | Unclassified - Protein with | | | |
| | | | | Unknown Function | | | |
| 617 | Gma.4093.1.S1_at | 0.33 | BI700897 | Transporter | | | |
| 618 | GmaAffx.17098.1.S1_at | 0.33 | BM084976 | Disease & Defense | | | |
| 619 | GmaAffx.33778.1.S1_at | 0.329 | CA819035 | Protein Destination & Storage | Storage Protein | | |
| 620 | Gma.6550.3.S1_s_at | 0.329 | AW234450 | Metabolism | | | Hydroxyindole-O-methyltransferase and |
| | | | | | | | related SAM-dependent |
| | | | | | | | methyltransferases |
| 621 | Gma.1622.1.A1_s_at | 0.329 | BU090523 | Signal Transduction | Protein Kinase | | FOG: Transposon-encoded proteins with |
| | | | | | | | TYA, reverse transcriptase, integrase |
| | | | | | | | domains in various combinations |
| 622 | Gma.7107.1.S1_at | 0.329 | AI855972 | Unclassified - Protein with | | | |
| | | | | Unknown Function | | | |
| 623 | Gma.4340.2.S1_a_at | 0.328 | BE611760 | Secondary Metabolism | | | Hydroxyindole-O-methyltransferase and |
| | | | | | | | related SAM-dependent |
| | | | | | | | methyltransferases |
| 624 | Gma.4873.1.S1_at | 0.328 | BI969532 | Signal Transduction | Protein Kinase | | Serine/threonine protein kinase |
| 625 | Gma.13335.1.S1_at | 0.327 | CD417489 | Unclassified - Hypothetical | | | |
| | | | | Protein Supported by cDNA | | | |
| 626 | GmaAffx.2716.1.A1_at | 0.327 | BU547858 | Transcription | Transcription | Zinc Finger | Protein involved in mRNA turnover and |
| | | | | | Factor | | stability |
| 627 | Gma.6033.1.S1_at | 0.327 | BI967767 | Unclassified - Hypothetical | | | |
| | | | | Protein Supported by cDNA | | | |
| 628 | Gma.13418.3.S1_at | 0.327 | CA938391 | Transporter | | | |
| 629 | Gma.4897.1.S1_at | 0.326 | BI969641 | Unclassified - Protein with | | | Thioredoxin, nucleoredoxin and related |
| | | | | Unknown Function | | | proteins |
| 630 | Gma.2540.2.S1_s_at | 0.325 | BI787366 | No Homology to Known | | | |
| | | | | Proteins | | | |
| 631 | GmaAffx.93553.1.S1_s_at | 0.325 | CF809278 | Metabolism | | | |
| 632 | Gma.13296.3.S1_at | 0.324 | CD418313 | Transporter | | | |
| 633 | GmaAffx.91254.1.S1_x_at | 0.324 | CF806979 | Unclassified - Hypothetical | | | |
| | | | | Protein Supported by cDNA | | | |
| 634 | GmaAffx.33677.1.S1_at | 0.324 | CA782582 | No Homology to Known | | | |
| | | | | Proteins | | | |
| 635 | GmaAffx.84144.1.A1_s_at | 0.324 | CD414061 | No Homology to Known | | | |
| | | | | Proteins | | | |
| 636 | GmaAffx.63553.1.S1_at | 0.324 | BG352380 | Energy | | | Cytochrome b |
| 637 | GmaAffx.93189.1.S1_s_at | 0.324 | CF808914 | Metabolism | | | Carbon-nitrogen hydrolase |
| 638 | GmaAffx.47819.1.A1_s_at | 0.324 | CD416142 | Unclassified - Hypothetical | | | Predicted mitochondrial ribosomal |
| | | | | Protein Supported by cDNA | | | protein L23 |
| 639 | Gma.5097.1.A1_at | 0.323 | BI969945 | Unclassified - Hypothetical | | | |
| | | | | Protein Supported by cDNA | | | |
| 640 | Gma.1895.1.A1_at | 0.323 | AW309584 | Protein Destination & Storage | | | ER lumen protein retaining receptor |
| 641 | Gma.12393.3.S1_at | 0.323 | BM085488 | Secondary Metabolism | | | Beta, beta-carotene 15,15′-dioxygenase |
| | | | | | | | and related enzymes |
| 642 | Gma.15359.2.S1_a_at | 0.322 | AW781747 | Intracellular Traffic | | | Predicted Ca2+-dependent phospholipid- |
| | | | | | | | binding protein |
| 643 | GmaAffx.84566.1.S1_x_at | 0.322 | BI786849 | Transcription | Transcription | MYB | Transcription factor, Myb superfamily |
| | | | | | Factor | | |
| 644 | Gma.12211.2.S1_at | 0.322 | BQ299492 | Disease & Defense | | | |
| 645 | Gma.17805.1.A1_s_at | 0.321 | BI968729 | Disease & Defense | | | |
| 646 | Gma.5950.1.S1_s_at | 0.321 | BQ610563 | Disease & Defense | | | |
| 647 | GmaAffx.70959.1.S1_at | 0.32 | BE611219 | Unclassified - Hypothetical | | | Glucose-6-phosphate/phosphate and |
| | | | | Protein NOT Supported by | | | phosphoenolpyruvate/phosphate |
| | | | | cDNA | | | antiporter |
| 648 | Gma.8484.1.S1_at | 0.32 | AF197946.1 | Signal Transduction | Protein Kinase | | |
| 649 | Gma.6213.1.S1_s_at | 0.32 | CD401652 | Protein Destination & Storage | | | Glutathione S-transferase |
| 650 | Gma.1394.2.S1_a_at | 0.32 | B1470370 | Metabolism | | | |
| 651 | GmaAffx.24350.1.A1_at | 0.32 | BU544060 | No Homology to Known | | | |
| | | | | Proteins | | | |
| 652 | GmaAffx.81287.1.S1_at | 0.319 | BG789983 | Secondary Metabolism | | | Hydroxyindole-O-methyltransferase and |
| | | | | | | | related SAM-dependent |
| | | | | | | | methyltransferases |
| 653 | Gma.13361.1.S1_at | 0.318 | AW309932 | Unclassified - Protein with | | | |
| | | | | Unknown Function | | | |
| 654 | Gma.2712.1.S1_at | 0.318 | AJ003246.1 | Secondary Metabolism | | | Flavonol reductase/cinnamoyl-CoA |
| | | | | | | | reductase |
| 655 | GmaAffx.89867.1.A1_s_at | 0.317 | CK606556 | Metabolism | | | Predicted PhzC/PhzF-type epimerase |
| 656 | Gma.2302.1.S1_x_at | 0.317 | AW307490 | Disease & Defense | | | Cysteine proteinase Cathepsin L |
| 657 | Gma.1573.2.S1_a_at | 0.316 | B1974098 | Metabolism | | | |
| 658 | GmaAffx.66301.1.S1_at | 0.316 | BG510223 | Secondary Metabolism | Ethylene | | Iron/ascorbate family oxidoreductases |
| | | | | | Biosynthesis | | |
| 659 | GmaAffx.72986.1.S1_at | 0.315 | BI317536 | Metabolism | | | Inositol polyphosphate 5-phosphatase |
| | | | | | | | and related proteins |
| 660 | GmaAffx.1620.3.S1_s_at | 0.315 | AW132226 | No Homology to Known | | | |
| | | | | Proteins | | | |
| 661 | GmaAffx.80098.1.S1_at | 0.315 | BG042861 | Metabolism | | | Arylacetamide deacetylase |
| 662 | Gma.9013.1.S1_at | 0.315 | AF243368.1 | Disease & Defense | | | Glutathione S-transferase |
| 663 | Gma.821.1.S1_at | 0.315 | AF243371.1 | Metabolism | | | Glutathione S-transferase |
| 664 | Gma.16940.1.S1_at | 0.315 | AW310039 | Transcription | Transcription | Homeodomain | Transcription factor ME1S1 and related |
| | | | | | Factor | | HOX domain proteins |
| 665 | Gma.10950.1.S1_at | 0.315 | B1699798 | Unclassified - Hypothetical | | | |
| | | | | Protein Supported by cDNA | | | |
| 666 | Gma.157.1.S1_at | 0.314 | D86351.1 | Metabolism | Cytochrome | CYP93D | Cytochrome P450 CYP2 subfamily |
| | | | | | P450 | | |
| 667 | GmaAffx.26967.1.S1_at | 0.314 | BE022958 | Unclassified - Hypothetical | | | |
| | | | | Protein Supported by cDNA | | | |
| 668 | GmaAffx.34798.1.S1_at | 0.314 | BM143105 | Unclassified - Hypothetical | | | |
| | | | | Protein Supported by cDNA | | | |
| 669 | GmaAffx.53845.1.S1_at | 0.313 | BG406552 | Transporter | | | Sulfate/bicarbonate/oxalate exchanger |
| | | | | | | | SAT-1 and related transporters (SLC26 |
| | | | | | | | family) |
| 670 | GmaAffx.28539.1.S1_at | 0.313 | BQ080184 | Disease & Defense | | | Ca2+-independent phospholipase A2 |
| 671 | Gma.11205.1.S1_a_at | 0.313 | BF066553 | Metabolism | | | Transketolase |
| 672 | GmaAffx.9127.1.S1_s_at | 0.313 | BI317582 | Transcription | Transcription | Zinc Finger | FOG: Predicted E3 ubiquitin ligase |
| | | | | | Factor | | |
| 673 | GmaAffx.3706.1.S1_at | 0.313 | BE821261 | Unclassified - Hypothetical | | | HGG motif-containing thioesterase |
| | | | | Protein NOT Supported by | | | |
| | | | | cDNA | | | |
| 674 | Gma.4526.1.S1_at | 0.312 | B1967711 | Transcription | Transcription | Zinc Finger | FOG: Zn-finger |
| | | | | | Factor | | |
| 675 | Gma.1704.1.S1_at | 0.312 | AW349971 | Metabolism | | | Glutaredoxin and related proteins |
| 676 | Gma.398.2.S1_at | 0.312 | BU925900 | Metabolism | | | Homocysteine S-methyltransferase |
| 677 | GmaAffx.82564.1.S1_s_at | 0.312 | BG047418 | No Homology to Known | | | |
| | | | | Proteins | | | |
| 678 | Gma.9622.1.S1_x_at | 0.311 | BQ610840 | Metabolism | | | Hydroxyindole-O-methyltransferase and |
| | | | | | | | related SAM-dependent |
| | | | | | | | methyltransferases |
| 679 | Gma.9205.1.S1_at | 0.31 | BM528605 | Unclassified - Hypothetical | | | |
| | | | | Protein Supported by cDNA | | | |
| 680 | Gma.2776.1.S1_at | 0.31 | BI968513 | Secondary Metabolism | Ethylene | | Iron/ascorbate family oxidoreductases |
| | | | | | Biosynthesis | | |
| 681 | Gma.6550.2.S1_x_at | 0.309 | BM086044 | Metabolism | | | Hydroxyindole-O-methyltransferase and |
| | | | | | | | related SAM-dependent |
| | | | | | | | methyltransferases |
| 682 | Gma.3581.2.S1_x_at | 0.309 | CD398783 | Unclassified - Hypothetical | | | Ca2+-dependent lipid-binding protein |
| | | | | Protein NOT Supported by | | | CLB1/vesicle protein |
| | | | | cDNA | | | vp115/Granuphilin A, contains C2 |
| | | | | | | | domain |
| 683 | GmaAffx.75594.1.S1_at | 0.309 | AW307139 | Secondary Metabolism | | | Multicopper oxidases |
| 684 | GmaAffx.14030.1.S1_at | 0.308 | BE021503 | No Homology to Known | | | |
| | | | | Proteins | | | |
| 685 | GmaAffx.87101.1.S1_at | 0.308 | BM309451 | No Homology to Known | | | |
| | | | | Proteins | | | |
| 686 | GmaAffx.55362.1.S1_at | 0.308 | BE475283 | Unclassified - Hypothetical | | | Reticulon |
| | | | | Protein Supported by cDNA | | | |
| 687 | Gma.79.4.S1_s_at | 0.308 | AI443819 | Unclassified - Hypothetical | | | |
| | | | | Protein Supported by cDNA | | | |
| 688 | Gma.8556.1.S1_at | 0.308 | BU764573 | Transporter | | | Amino acid transporters |
| 689 | GmaAffx.82195.1.S1_at | 0.308 | BM528489 | Unclassified - Hypothetical | | | |
| | | | | Protein NOT Supported by | | | |
| | | | | cDNA | | | |
| 690 | Gma.3450.1.S1_at | 0.307 | BE657895 | Unclassified - Hypothetical | | | |
| | | | | Protein Supported by cDNA | | | |
| 691 | GmaAffx.64261.1.A1_at | 0.307 | B1969080 | Unclassified - Protein with | | | 1,4-benzoquinone reductase-like; Trp |
| | | | | Unknown Function | | | repressor binding protein-like/protoplast- |
| | | | | | | | secreted protein |
| 692 | GmaAffx.90515.1.S1_s_at | 0.306 | CF806240 | Unclassified - Hypothetical | | | Predicted pyroglutamyl peptidase |
| | | | | Protein Supported by cDNA | | | |
| 693 | Gma.4578.1.S1_s_at | 0.306 | BE802809 | Transporter | | | Plasma membrane H+-transporting |
| | | | | | | | ATPase |
| 694 | GmaAffx.47739.1.S1_at | 0.305 | BM522736 | Unclassified - Protein with | | | |
| | | | | Unknown Function | | | |
| 695 | Gma.3161.2.S1_at | 0.305 | CD400025 | Secondary Metabolism | | | Iron/ascorbate family oxidoreductases |
| 696 | GmaAffx.93116.1.S1_at | 0.305 | CF806290 | Disease & Defense | | | |
| 697 | GmaAffx.5255.1.S1_at | 0.305 | BG362929 | Transcription | Transcription | AP2/EREBP | |
| | | | | | Factor | | |
| 698 | Gma.3976.1.S1_at | 0.304 | BI972268 | No Homology to Known | | | |
| | | | | Proteins | | | |
| 699 | Gma.12641.1.A1_at | 0.304 | CD393495 | No Homology to Known | | | |
| | | | | Proteins | | | |
| 700 | GmaAffx.72479.1.S1_at | 0.303 | BG043825 | Transcription | Transcription | AP2/EREBP | Uncharacterized conserved protein |
| | | | | | Factor | | |
| 701 | GmaAffx.48438.1.S1_at | 0.302 | AW350858 | Protein Destination & Storage | | | Molecular chaperone (DnaJ superfamily) |
| 702 | Gma.6550.2.S1_s_at | 0.302 | BM086044 | Metabolism | | | Hydroxyindole-O-methyltransferase and |
| | | | | | | | related SAM-dependent |
| | | | | | | | methyltransferases |
| 703 | GmaAffx.57748.1.S1_at | 0.302 | AW306274 | No Homology to Known | | | |
| | | | | Proteins | | | |
| 704 | Gma.3751.1.S1_at | 0.301 | AW350303 | Unclassified - Hypothetical | | | |
| | | | | Protein NOT Supported by | | | |
| | | | | cDNA | | | |
| 705 | GmaAffx.16207.1.S1_at | 0.301 | BE821188 | Cell Structure | | | Beta-glucosidase, lactase |
| | | | | | | | phlorizinhydrolase, and related proteins |
| 706 | Gma.15359.1.S1_x_at | 0.3 | BM271008 | Unclassified - Protein with | | | Predicted Ca2+-dependent phospholipid- |
| | | | | Unknown Function | | | binding protein |
| 707 | GmaAffx.60672.1.S1_at | 0.299 | BU080558 | No Homology to Known | | | |
| | | | | Proteins | | | |
| 708 | Gma.6462.2.S1_a_at | 0.299 | BE555263 | Unclassified - Hypothetical | | | |
| | | | | Protein NOT Supported by | | | |
| | | | | cDNA | | | |
| 709 | GmaAffx.9926.1.A1_at | 0.299 | CD411291 | No Homology to Known | | | |
| | | | | Proteins | | | |
| 710 | GmaAffx.78297.1.S1_at | 0.299 | BM731185 | Unclassified - Hypothetical | | | FOG: Predicted E3 ubiquitin ligase |
| | | | | Protein Supported by cDNA | | | |
| 711 | Gma.2302.1.S1_at | 0.299 | AW307490 | Disease & Defense | | | Cysteine proteinase Cathepsin L |
| 712 | Gma.73.1.S1_at | 0.298 | AF098662.1 | Protein Destination & Storage | | | |
| 713 | Gma.6151.1.S1_at | 0.298 | BQ611806 | Metabolism | | | Flavin-containing monooxygenase |
| 714 | Gma.17610.1.S1_at | 0.298 | U50075.1 | Metabolism | | | |
| 715 | Gma.13187.1.S1_at | 0.298 | BG839239 | Unclassified - Hypothetical | | | Uncharacterized conserved protein |
| | | | | Protein Supported by cDNA | | | |
| 716 | GmaAffx.76696.1.A1_at | 0.297 | BE824386 | No Homology to Known | | | |
| | | | | Proteins | | | |
| 717 | GmaAffx.89060.1.S1_at | 0.296 | CK605664 | Metabolism | Cytochrome | CYP93D | Cytochrome P450 CYP2 subfamily |
| | | | | | P450 | | |
| 718 | GmaAffx.89772.10.A1_s_at | 0.296 | CK606097 | Disease & Defense | | | |
| 719 | GmaAffx.92112.1.S1_s_at | 0.295 | CF807837 | No Homology to Known | | | |
| | | | | Proteins | | | |
| 720 | Gma.15093.1.S1_at | 0.295 | CD400374 | Protein Destination & Storage | | | Glutathione S-transferase |
| 721 | GmaAffx.93157.1.S1_s_at | 0.295 | CF808882 | Metabolism | | | Aldo/keto reductase family proteins |
| 722 | GmaAffx.89075.1.S1_s_at | 0.295 | CK606454 | No Homology to Known | | | |
| | | | | Proteins | | | |
| 723 | Gma.6623.1.S1_at | 0.294 | BI702268 | Unclassified - Protein with | | | 1,4-benzoquinone reductase-like; Trp |
| | | | | Unknown Function | | | repressor binding protein-like/protoplast- |
| | | | | | | | secreted protein |
| 724 | Gma.4305.1.S1_at | 0.294 | AW350687 | Disease & Defense | | | |
| 725 | GmaAffx.38555.2.S1_at | 0.294 | B1425873 | No Homology to Known | | | |
| | | | | Proteins | | | |
| 726 | GmaAffx.91037.1.S1_s_at | 0.294 | CF806762 | Secondary Metabolism | | | Methyltransferase |
| 727 | GmaAffx.74300.1.S1_at | 0.294 | BU080963 | Disease & Defense | | | |
| 728 | GmaAffx.18113.2.S1_s_at | 0.294 | AW706795 | Unclassified - Protein with | Hormone | | |
| | | | | Unknown Function | Induced | | |
| 729 | GmaAffx.12014.1.A1_at | 0.294 | CA802431 | Unclassified - Hypothetical | | | NADH pyrophosphatase 1 of the Nudix |
| | | | | Protein Supported by cDNA | | | family of hydrolases |
| 730 | GmaAffx.89772.8.S1_at | 0.294 | CK605778 | Metabolism | | | |
| 731 | GmaAffx.61311.1.S1_at | 0.294 | CA936951 | Transporter | | | Amino acid transporters |
| 732 | GmaAffx.42732.1.S1_at | 0.294 | BU765857 | Protein Destination & Storage | Ubiquitin | | FOG: Predicted E3 ubiquitin ligase |
| | | | | | Degradation | | |
| 733 | GmaAffx.50677.1.S1_s_at | 0.293 | B1971744 | No Homology to Known | | | |
| | | | | Proteins | | | |
| 734 | Gma.3963.2.S1_at | 0.293 | BE608192 | Unclassified - Hypothetical | | | Zn-finger protein |
| | | | | Protein NOT Supported by | | | |
| | | | | cDNA | | | |
| 735 | Gma.2718.1.S1_at | 0.293 | CD406302 | Transporter | | | Multidrug resistance-associated |
| | | | | | | | protein/mitoxantrone resistance protein, |
| | | | | | | | ABC superfamily |
| 736 | Gma.6640.1.S1_at | 0.292 | BQ628278 | Disease & Defense | | | |
| 737 | Gma.7454.1.S1_a_at | 0.292 | CD405901 | Transcription | Transcription | AUX/IAA | |
| | | | | | Factor | | |
| 738 | Gma.9236.1.S1_at | 0.291 | BG405642 | Metabolism | Amino Acid | | Spermidine synthase |
| | | | | | Biosynthesis/ | | |
| | | | | | Metabolism | | |
| 739 | Gma.6895.1.S1_a_at | 0.291 | BU545788 | Metabolism | | | Alanine-glyoxylate aminotransferase |
| | | | | | | | AGT2 |
| 740 | Gma.5733.1.S1_at | 0.291 | CD402671 | Signal Transduction | Calcium | | Ca2+-binding protein |
| | | | | | Binding | | (centrin/caltractin), EF-Hand |
| | | | | | | | superfamily protein |
| 741 | GmaAffx.18953.1.S1_at | 0.291 | BM178798 | No Homology to Known | | | |
| | | | | Proteins | | | |
| 742 | GmaAffx.90703.1.A1_at | 0.29 | CF809087 | Disease & Defense | | | |
| 743 | GmaAffx.79390.2.S1_at | 0.29 | AW597383 | Transcription | Transcription | Basic Leucine | FOG: Armadillo/beta-catenin-like |
| | | | | | Factor | Zipper (bZIP) | repeats |
| 744 | Gma.6310.1.S1_at | 0.29 | BQ612831 | Signal Transduction | MAPKKK | MEKK | MEKK and related serine/threonine |
| | | | | | | | protein kinases |
| 745 | GmaAffx.31838.1.S1_at | 0.289 | BU547835 | Secondary Metabolism | Flavonoid | | Flavonol reductase/cinnamoyl-CoA |
| | | | | | Biosynthesis | | reductase |
| 746 | Gma.15605.1.S1_at | 0.288 | BM143798 | Transcription | Transcription | AP2/EREBP | |
| | | | | | Factor | | |
| 747 | Gma.17835.1.S1_at | 0.288 | BE822840 | Metabolism | | | |
| 748 | Gma.8394.1.S1_at | 0.288 | CD392332 | Disease & Defense | | | Prohibitins and stomatins of the P1D |
| | | | | | | | superfamily |
| 749 | Gtna.6264.1.S1_at | 0.288 | BQ612558 | Unclassified - Hypothetical | | | |
| | | | | Protein Supported by cDNA | | | |
| 750 | Gma.6630.1.S1_s_at | 0.288 | BG652851 | Unclassified - Hypothetical | | | |
| | | | | Protein Supported by cDNA | | | |
| 751 | Gma.2388.1.S1_at | 0.288 | CD416539 | No Homology to Known | | | |
| | | | | Proteins | | | |
| 752 | GmaAffx.727.1.S1_at | 0.287 | BE804565 | Unclassified - Protein with | | | Ubiquinone biosynthesis |
| | | | | Unknown Function | | | methyltransferase COQ5 |
| 753 | Gma.13000.1.S1_at | 0.287 | CD415111 | Unclassified - Hypothetical | | | |
| | | | | Protein Supported by cDNA | | | |
| 754 | GmaAffx.11049.1.S1_at | 0.287 | BU545140 | Unclassified - Protein with | | | Phosphatidylethanolamine binding |
| | | | | Unknown Function | | | protein |
| 755 | Gma.11731.1.A1_at | 0.287 | CD392320 | Unclassified - Protein with | | | |
| | | | | Unknown Function | | | |
| 756 | Gma.5578.1.S1_x_at | 0.287 | BE659192 | Metabolism | Cytochrome | CYP98A | Cytochrome P450 CYP2 subfamily |
| | | | | | P450 | | |
| 757 | GmaAffx.93607.1.S1_s_at | 0.286 | CF809332 | Unclassified - Protein with | | | |
| | | | | Unknown Function | | | |
| 758 | GmaAffx.91185.1.S1_at | 0.286 | CF806910 | Cell Structure | | | Pectin acetylesterase and similar |
| | | | | | | | proteins |
| 759 | Gma.11277.2.S1_a_at | 0.286 | BI701238 | Unclassified - Hypothetical | | | |
| | | | | Protein NOT Supported by | | | |
| | | | | cDNA | | | |
| 760 | Gma.6033.2.S1_at | 0.286 | AI416728 | Unclassified - Hypothetical | | | |
| | | | | Protein Supported by cDNA | | | |
| 761 | GmaAffx.478.2.S1_at | 0.285 | BU763963 | Transcription | Transcription | Homeodomain | Transcription factor MEIS1 and related |
| | | | | | Factor | | HOX domain proteins |
| 762 | GmaAffx.71711.1.S1_at | 0.285 | AI988272 | No Homology to Known | | | |
| | | | | Proteins | | | |
| 763 | Gma.3345.1.S1_at | 0.285 | AW394524 | Metabolism | | | Alcohol dehydrogenase, class III |
| 764 | GmaAffx.84338.1.S1_at | 0.285 | AI856789 | Unclassified - Hypothetical | | | Predicted E3 ubiquitin ligase |
| | | | | Protein Supported by cDNA | | | |
| 765 | GmaAffx.90069.1.S1_at | 0.284 | CF805794 | Unclassified - Hypothetical | | | Leucine rich repeat proteins, some |
| | | | | Protein Supported by cDNA | | | proteins contain F-box |
| 766 | GmaAffx.75697.1.S1_s_at | 0.284 | BU090518 | Unclassified - Hypothetical | | | |
| | | | | Protein Supported by cDNA | | | |
| 767 | Gma.6187.1.S1_at | 0.283 | BF324343 | No Homology to Known | | | |
| | | | | Proteins | | | |
| 768 | Gma.12211.1.S1_at | 0.283 | AW394548 | Disease & Defense | | | |
| 769 | Gma.3880.1.S1_at | 0.283 | BE659305 | Unclassified - Hypothetical | | | |
| | | | | Protein NOT Supported by | | | |
| | | | | cDNA | | | |
| 770 | Gma.4822.1.S1_at | 0.283 | B1969249 | Disease & Defense | | | |
| 771 | GmaAffx.32328.1.A1_at | 0.283 | B1968685 | No Homology to Known | | | |
| | | | | Proteins | | | |
| 772 | Gma.3963.1.S1_at | 0.283 | AW202007 | Unclassified - Hypothetical | | | Zn-finger protein |
| | | | | Protein NOT Supported by | | | |
| | | | | cDNA | | | |
| 773 | GmaAffx.56606.1.S1_at | 0.283 | BG507644 | Secondary Metabolism | | | Multicopper oxidases |
| 774 | GmaAffx.47288.1.S1_at | 0.282 | BU764979 | Unclassified - Hypothetical | | | GTPase Rab18, small G protein |
| | | | | Protein Supported by cDNA | | | superfamily |
| 775 | Gma.850.1.S1_at | 0.281 | BE822050 | Unclassified - Hypothetical | | | |
| | | | | Protein NOT Supported by | | | |
| | | | | cDNA | | | |
| 776 | Gma.5634.1.S1_s_at | 0.28 | BE806891 | Secondary Metabolism | | | Steroid reductase |
| 777 | Gma.1332.1.S1_s_at | 0.279 | BI970666 | Secondary Metabolism | Ethylene | | 1-aminocyclopropane-1-carboxylate |
| | | | | | Biosynthesis | | synthase, and related proteins |
| 778 | Gma.1068.1.S1_at | 0.279 | L19360.1 | Signal Transduction | Protein Kinase | | Serine/threonine protein kinase |
| 779 | Gma.2590.1.A1_s_at | 0.279 | CD406617 | Disease & Defense | | | |
| 780 | Gma.6047.1.S1_s_at | 0.278 | BQ611159 | Unclassified - Protein with | | | |
| | | | | Unknown Function | | | |
| 781 | Gma.1716.1.S1_at | 0.278 | AW156801 | Unclassified - Hypothetical | | | |
| | | | | Protein Supported by cDNA | | | |
| 782 | GmaAffx.50646.1.A1_at | 0.277 | AW310451 | Unclassified - Hypothetical | | | Molybdenum cofactor sulfurase |
| | | | | Protein Supported by cDNA | | | |
| 783 | Gma.3591.2.S1_at | 0.277 | BE473658 | Secondary Metabolism | Ethylene | | Iron/ascorbate family oxidoreductases |
| | | | | | Biosynthesis | | |
| 784 | GmaAffx.90652.1.S1_at | 0.276 | CF806377 | Unclassified - Hypothetical | | | Predicted hydrolase/acyltransferase |
| | | | | Protein Supported by cDNA | | | (alpha/beta hydrolase superfamily) |
| 785 | Gma.17015.1.S1_at | 0.275 | AW733363 | No Homology to Known | | | |
| | | | | Proteins | | | |
| 786 | GmaAffx.80830.1.A1_at | 0.275 | BU549591 | Unclassified - Hypothetical | | | |
| | | | | Protein Supported by cDNA | | | |
| 787 | Gma.7494.1.S1_at | 0.275 | BU548105 | Signal Transduction | Protein | | Serine/threonine protein phosphatase |
| | | | | | Phosphatase | | |
| 788 | GmaAffx.79516.1.A1_at | 0.275 | CD400596 | Transcription | Transcription | MYB | |
| | | | | | Factor | | |
| 789 | Gma.4593.1.S1_a_at | 0.275 | CD396298 | Energy | | | |
| 790 | Gma.8514.1.S1_at | 0.275 | CA851666 | Protein Destination & Storage | | | Glutathione S-transferase |
| 791 | GmaAffx.85528.1.S1_at | 0.274 | BE608070 | Unclassified - Hypothetical | | | FOG: PPR repeat |
| | | | | Protein Supported by cDNA | | | |
| 792 | Gma.6763.1.A1_at | 0.273 | BU544794 | Energy | | | Ferric reductase, NADH/NADPH |
| | | | | | | | oxidase and related proteins |
| 793 | Gma.6623.2.S1_s_at | 0.273 | BQ629284 | Unclassified - Protein with | | | 1,4-benzoquinone reductase-like; Trp |
| | | | | Unknown Function | | | repressor binding protein-like/protoplast- |
| | | | | | | | secreted protein |
| 794 | GmaAffx.68507.1.S1_at | 0.273 | B1699405 | Transcription | Transcription | Heat Shock | Heat shock transcription factor |
| | | | | | Factor | | |
| 795 | GmaAffx.80393.1.A1_at | 0.272 | BE658680 | No Homology to Known | | | |
| | | | | Proteins | | | |
| 796 | GmaAffx.92568.1.S1_at | 0.272 | CF808293 | Metabolism | | | Acyl-CoA-binding protein |
| 797 | Gma.8022.1.S1_at | 0.272 | BQ628998 | Disease & Defense | | | Soluble epoxide hydrolase |
| 798 | Gma.9534.1.S1_at | 0.272 | CA802874 | Disease & Defense | | | |
| 799 | GmaAffx.36470.1.A1_at | 0.272 | BU550552 | Unclassified - Hypothetical | | | |
| | | | | Protein Supported by cDNA | | | |
| 800 | Gma.11284.1.S1_at | 0.272 | AW153035 | Disease & Defense | | | |
| 801 | Gma.6999.1.S1_x_at | 0.271 | AF529303.1 | Disease & Defense | | | |
| 802 | GmaAffx.64967.1.S1_at | 0.271 | BU546115 | Secondary Metabolism | | | Iron/ascorbate family oxidoreductases |
| 803 | Gma.6550.1.A1_at | 0.271 | CD402638 | Metabolism | | | Hydroxyindole-O-methyltransferase and |
| | | | | | | | related SAM-dependent |
| | | | | | | | methyltransferases |
| 804 | GmaAffx.l5471.1.S1_at | 0.271 | BQ611747 | Transcription | Transcription | MYB | Transcription factor, Myb superfamily |
| | | | | | Factor | | |
| 805 | Gma.15155.2.S1_at | 0.27 | BG789619 | Unclassified - Hypothetical | | | |
| | | | | Protein Supported by cDNA | | | |
| 806 | Gma.426.1.A1_at | 0.27 | AI442768 | Transcription | Transcription | Zinc Finger | |
| | | | | | Factor | | |
| 807 | Gma.17300.1.A1_at | 0.27 | AW350946 | Unclassified - Hypothetical | | | |
| | | | | Protein NOT Supported by | | | |
| | | | | cDNA | | | |
| 808 | GmaAffx.25612.1.S1_at | 0.269 | AW317577 | No Homology to Known | | | |
| | | | | Proteins | | | |
| 809 | GmaAffx.33527.1.S1_at | 0.268 | BU761195 | No Homology to Known | | | |
| | | | | Proteins | | | |
| 810 | GmaAffx.11999.1.S1_at | 0.267 | AW567661 | No Homology to Known | | | |
| | | | | Proteins | | | |
| 811 | Gma.4093.2.S1_at | 0.267 | BQ081460 | Transporter | | | |
| 812 | Gma.11277.2.S1_at | 0.267 | B1701238 | Unclassified - Hypothetical | | | |
| | | | | Protein NOT Supported by | | | |
| | | | | cDNA | | | |
| 813 | Gma.398.1.A1_at | 0.267 | AW350679 | Metabolism | | | Homocysteine S-methyltransferase |
| 814 | GmaAffx.91266.1.S1_s_at | 0.267 | CF806991 | Unclassified - Hypothetical | | | |
| | | | | Protein Supported by cDNA | | | |
| 815 | GmaAffx.1301.59.S1_at | 0.266 | B1974591 | Intracellular Traffic | | | Annexin |
| 816 | Gma.1137.1.S1_a_at | 0.266 | BF067710 | Metabolism | | | |
| 817 | GmaAffx.45805.2.S1_at | 0.265 | CA784263 | Metabolism | | | Hydroxyindole-O-methyltransferase and |
| | | | | | | | related SAM-dependent |
| | | | | | | | methyltransferases |
| 818 | GmaAffx.84566.1.S1_at | 0.265 | BI786849 | Transcription | Transcription | MYB | Transcription factor, Myb superfamily |
| | | | | | Factor | | |
| 819 | GmaAffx.69198.1.S1_at | 0.264 | BM526951 | Unclassified - Hypothetical | | | Leucine rich repeat proteins, some |
| | | | | Protein Supported by cDNA | | | proteins contain F-box |
| 820 | GmaAffx.4888.1.A1_at | 0.264 | B1967635 | No Homology to Known | | | |
| | | | | Proteins | | | |
| 821 | Gma.14897.2.S1_at | 0.263 | BQ785154 | Unclassified - Protein with | | | Dihydroorotase and related enzymes |
| | | | | Unknown Function | | | |
| 822 | Gma.8515.1.S1_at | 0.263 | AF243372.1 | Protein Destination & Storage | | | Glutathione S-transferase |
| 823 | GmaAffx.53914.1.A1_at | 0.262 | BE822062 | No Homology to Known | | | |
| | | | | Proteins | | | |
| 824 | Gma.1758.1.S1_at | 0.262 | CD402046 | Unclassified - Protein with | | | |
| | | | | Unknown Function | | | |
| 825 | Gma.4470.1.S1_s_at | 0.262 | AW156251 | Unclassified - Protein with | | | |
| | | | | Unknown Function | | | |
| 826 | GmaAffx.86927.1.S1_at | 0.261 | CA937162 | No Homology to Known | | | |
| | | | | Proteins | | | |
| 827 | GmaAffx.60958.1.S1_at | 0.261 | BG551038 | Transcription | Transcription | Basic Helix-Loop- | |
| | | | | | Factor | Helix (bHLH) | |
| 828 | Gma.3893.3.S1_at | 0.261 | BG651854 | Unclassified - Hypothetical | | | |
| | | | | Protein Supported by cDNA | | | |
| 829 | Gma.7580.1.S1_at | 0.261 | BE057763 | Unclassified - Hypothetical | | | |
| | | | | Protein Supported by cDNA | | | |
| 830 | GmaAffx.32345.1.A1_s_at | 0.261 | BI970277 | Cell Structure | | | |
| 831 | GmaAffx.86247.1.S1_s_at | 0.26 | BG238674 | Unclassified - Hypothetical | | | |
| | | | | Protein Supported by cDNA | | | |
| 832 | Gma.15359.1.S1_a_at | 0.26 | BM271008 | Unclassified - Protein with | | | Predicted Ca2+-dependent phospholipid- |
| | | | | Unknown Function | | | binding protein |
| 833 | GmaAffx.12933.1.S1_at | 0.26 | AW395787 | Transcription | Transcription | CCAAT | CCAAT-binding factor, subunit B |
| | | | | | Factor | | (HAP2) |
| 834 | GmaAffx.1301.68.S1_s_at | 0.26 | BI974374 | Intracellular Traffic | | | Annexin |
| 835 | GmaAffx.84424.1.S1_at | 0.259 | AW311543 | Metabolism | | | Alcohol dehydrogenase, class V |
| 836 | Gma.5578.3.S1_a_at | 0.259 | AW310572 | Metabolism | Cytochrome | CYP98A | Cytochrome P450 CYP2 subfamily |
| | | | | | P450 | | |
| 837 | GmaAffx.76821.1.S1_at | 0.259 | BG238158 | Unclassified - Protein with | Hormone | | |
| | | | | Unknown Function | Induced | | |
| 838 | GmaAffx.91328.1.S1_s_at | 0.258 | CF807053 | Unclassified - Hypothetical | | | Leucine rich repeat proteins, some |
| | | | | Protein Supported by cDNA | | | proteins contain F-box |
| 839 | GmaAffx.32606.1.S1_at | 0.258 | BM270707 | Unclassified - Hypothetical | | | Aquaporin (major intrinsic protein |
| | | | | Protein NOT Supported by | | | family) |
| | | | | cDNA | | | |
| 840 | GmaAffx.40015.1.S1_at | 0.258 | BE609673 | No Homology to Known | | | |
| | | | | Proteins | | | |
| 841 | GmaAffx.50613.1.A1_at | 0.258 | B1967418 | No Homology to Known | | | |
| | | | | Proteins | | | |
| 842 | Gma.7102.1.S1_a_at | 0.258 | AW309275 | Unclassified - Hypothetical | | | Leucine rich repeat proteins, some |
| | | | | Protein Supported by cDNA | | | proteins contain F-box |
| 843 | GmaAffx.77203.1.S1_at | 0.258 | B1970687 | Unclassified - Hypothetical | | | |
| | | | | Protein Supported by cDNA | | | |
| 844 | Gma.3000.1.S1_at | 0.257 | BE657172 | No Homology to Known | | | |
| | | | | Proteins | | | |
| 845 | Gma.728.1.A1_at | 0.257 | BE658779 | No Homology to Known | | | |
| | | | | Proteins | | | |
| 846 | Gma.1951.2.S1_a_at | 0.256 | CA783485 | Protein Destination & Storage | | | Serine carboxypeptidases |
| 847 | GmaAffx.45003.1.S1_at | 0.256 | BI968048 | Unclassified - Hypothetical | | | Uncharacterized conserved protein |
| | | | | Protein Supported by cDNA | | | |
| 848 | GmaAffx.20108.1.S1_at | 0.256 | CA801220 | Unclassified - Hypothetical | | | |
| | | | | Protein Supported by cDNA | | | |
| 849 | Gma.5634.1.S1_at | 0.256 | BE806891 | Secondary Metabolism | | | Steroid reductase |
| 850 | Gma.17516.1.S1_at | 0.255 | CD396417 | Transporter | | | Ca2+/H+ antiporter VCX1 and related |
| | | | | | | | proteins |
| 851 | GmaAffx.92148.1.S1_s_at | 0.255 | CF807873 | Unclassified - Hypothetical | | | |
| | | | | Protein NOT Supported by | | | |
| | | | | cDNA | | | |
| 852 | Gma.2080.2.S1_a_at | 0.255 | BM520916 | Secondary Metabolism | | | |
| 853 | GmaAffx.92136.1.S1_s_at | 0.254 | CF807451 | Signal Transduction | Protein Kinase | | Mannosyltransferase |
| 854 | GmaAffx.37803.2.S1_at | 0.254 | BE347017 | No Homology to Known | | | |
| | | | | Proteins | | | |
| 855 | GmaAffx.53324.1.A1_at | 0.254 | AW309251 | Transcription | Transcription | Basic Helix-Loop- | |
| | | | | | Factor | Helix (bHLH) | |
| 856 | GmaAffx.19118.1.S1_at | 0.253 | BM523731 | Unclassified - Hypothetical | | | |
| | | | | Protein NOT Supported by | | | |
| | | | | cDNA | | | |
| 857 | GmaAffx.51466.1.A1_at | 0.253 | CD406245 | Metabolism | | | UDP-glucuronosyl and UDP-glucosyl |
| | | | | | | | transferase |
| 858 | Gma.2102.1.S1_s_at | 0.253 | BM523392 | Disease & Defense | | | |
| 859 | GmaAffx.83172.1.S1_at | 0.253 | BQ610576 | Unclassified - Protein with | | | |
| | | | | Unknown Function | | | |
| 860 | GmaAffx.90069.1.S1_s_at | 0.253 | CF805794 | Unclassified - Hypothetical | | | Leucine rich repeat proteins, some |
| | | | | Protein Supported by cDNA | | | proteins contain F-box |
| 861 | Gma.6730.1.S1_s_at | 0.252 | BE211440 | Metabolism | | | UDP-glucuronosyl and UDP-glucosyl |
| | | | | | | | transferase |
| 862 | GmaAffx.85662.1.S1_at | 0.252 | BG404902 | Unclassified - Protein with | | | Predicted membrane protein, contains |
| | | | | Unknown Function | | | DoH and Cytochrome b-561/ferric |
| | | | | | | | reductase transmembrane domains |
| 863 | Gma.1619.1.S1_at | 0.252 | AW472195 | Unclassified - Hypothetical | | | |
| | | | | Protein Supported by cDNA | | | |
| 864 | Gma.1495.1.S1_at | 0.251 | B1973545 | No Homology to Known | | | |
| | | | | Proteins | | | |
| 865 | Gma.7836.1.A1_at | 0.251 | BU549650 | Unclassified - Hypothetical | | | |
| | | | | Protein NOT Supported by | | | |
| | | | | cDNA | | | |
| 866 | Gma.11277.1.S1_a_at | 0.251 | CA802310 | Protein Destination & Storage | | | |
| 867 | Gma.9086.2.S1_at | 0.251 | BE555169 | Cell Growth & Division | | | |
| 868 | GmaAffx.87547.2.S1_at | 0.251 | BQ080473 | Cell Structure | | | Beta-glucosidase, lactase |
| | | | | | | | phlorizinhydrolase, and related proteins |
| 869 | GmaAffx.47891.1.S1_s_at | 0.251 | BQ785880 | Cell Structure | | | |
| 870 | GmaAffx.50811.2.S1_at | 0.251 | BG725712 | Transcription | Transcription | NAC Domain | |
| | | | | | Factor | | |
| 871 | GmaAffx.72630.1.S1_at | 0.25 | BG239729 | Unclassified - Hypothetical | | | FOG: TPR repeat |
| | | | | Protein NOT Supported by | | | |
| | | | | cDNA | | | |
| 871 | GmaAffx.72630.1.S1_at | 0.25 | BG239729 | Unclassified - Hypothetical | | | Thioredoxin |
| | | | | Protein NOT Supported by | | | |
| | | | | cDNA | | | |
| 872 | Gma.8365.1.S1_at | 0.25 | CD396813 | Unclassified - Hypothetical | | | |
| | | | | Protein NOT Supported by | | | |
| | | | | cDNA | | | |
| 873 | Gma.9856.1.S1_at | 0.25 | BG650352 | Signal Transduction | Protein Kinase | | Serine/threonine protein kinase |
| 874 | Gma.8113.1.A1_at | 0.249 | BU548342 | No Homology to Known | | | |
| | | | | Proteins | | | |
| 875 | Gma.16875.1.A1_at | 0.248 | CD406723 | Metabolism | | | Aldo/keto reductase family proteins |
| 876 | Gma.4249.1.S1_at | 0.248 | BF324893 | Secondary Metabolism | | | Methyltransferase |
| 877 | GmaAffx.5743.1.S1_at | 0.247 | BQ628343 | Unclassified - Hypothetical | | | FOG: Predicted E3 ubiquitin ligase |
| | | | | Protein Supported by cDNA | | | |
| 878 | Gma.6719.2.S1_at | 0.247 | BG726773 | No Homology to Known | | | |
| | | | | Proteins | | | |
| 879 | Gma.358.1.S1_at | 0.247 | AW310270 | Metabolism | | | Arylacetamide deacetylase |
| 880 | Gma.1951.1.S1_at | 0.246 | BQ079657 | Protein Destination & Storage | | | Serine carboxypeptidases |
| 881 | GmaAffx.2026.1.A1_at | 0.246 | BU550139 | Transcription | Transcription | NAC Domain | |
| | | | | | Factor | | |
| 882 | Gma.10342.1.S1_at | 0.246 | BE058741 | Unclassified - Hypothetical | | | |
| | | | | Protein Supported by cDNA | | | |
| 883 | GmaAffx.82936.1.S1_at | 0.246 | AI938364 | Unclassified - Protein with | | | |
| | | | | Unknown Function | | | |
| 884 | GmaAffx.45957.2.S1_at | 0.246 | B1974135 | No Homology to Known | | | |
| | | | | Proteins | | | |
| 885 | GmaAffx.61872.1.S1_at | 0.245 | AW102386 | Signal Transduction | Protein Kinase | | |
| 886 | Gma.2439.1.S1_at | 0.244 | B1972053 | Metabolism | | | Predicted PhzC/PhzF-type epimerase |
| 887 | GmaAffx.24102.1.S1_at | 0.244 | BQ453911 | Unclassified - Hypothetical | | | |
| | | | | Protein Supported by cDNA | | | |
| 888 | GmaAffx.90395.1.A1_s_at | 0.244 | CF807499 | Unclassified - Protein with | | | |
| | | | | Unknown Function | | | |
| 889 | GmaAffx.15613.1.S1_at | 0.243 | AI939073 | Unclassified - Hypothetical | | | |
| | | | | Protein NOT Supported by | | | |
| | | | | cDNA | | | |
| 890 | GmaAffx.90331.1.S1_s_at | 0.243 | CF806056 | Disease & Defense | | | |
| 891 | GmaAffx.30421.1.S1_at | 0.243 | BU082092 | Transcription | Transcription | Zinc Finger | FOG: Predicted E3 ubiquitin ligase |
| | | | | | Factor | | |
| 892 | GmaAffx.22552.1.S1_at | 0.243 | BU549952 | Transporter | | | H+/oligopeptide symporter |
| 893 | GmaAffx.93301.2.S1_s_at | 0.242 | CF808497 | Secondary Metabolism | | | Flavonol reductase/cinnamoyl-CoA |
| | | | | | | | reductase |
| 894 | Gma.3531.1.S1_s_at | 0.241 | BI498803 | Unclassified - Hypothetical | | | FOG: PPR repeat |
| | | | | Protein Supported by cDNA | | | |
| 895 | Gma.4665.1.A1_at | 0.241 | AW309815 | No Homology to Known | | | |
| | | | | Proteins | | | |
| 896 | GmaAffx.24368.1.A1_at | 0.241 | BU545331 | No Homology to Known | | | |
| | | | | Proteins | | | |
| 897 | Gma.9086.1.S1_at | 0.24 | AWI84932 | Cell Growth & Division | | | |
| 898 | Gma.3579.1.S1_at | 0.239 | AW349799 | Unclassified - Hypothetical | | | Kelch repeat-containing proteins |
| | | | | Protein Supported by cDNA | | | |
| 899 | Gma.5586.2.S1_at | 0.239 | CD398384 | Transcription | Transcription | Unclassified | CCAAT-binding factor, subunit B |
| | | | | | Factor | | (HAP2) |
| 900 | Gma.6280.2.S1_at | 0.239 | AI938985 | No Homology to Known | | | |
| | | | | Proteins | | | |
| 901 | GmaAffx.66451.1.S1_at | 0.238 | AW309198 | Secondary Metabolism | | | |
| 902 | Gma.418.1.A1_at | 0.237 | CA784607 | Unclassified - Hypothetical | | | Glycosyl transferase, family 8 - |
| | | | | Protein Supported by cDNA | | | glycogenin |
| 903 | GmaAffx.91479.1.S1_s_at | 0.237 | CF807204 | No Homology to Known | | | |
| | | | | Proteins | | | |
| 904 | Gma.13910.1.S1_at | 0.237 | CD409745 | Unclassified - Protein with | | | |
| | | | | Unknown Function | | | |
| 905 | Gma.17890.1.S1_at | 0.236 | AW432965 | No Homology to Known | | | |
| | | | | Proteins | | | |
| 906 | GmaAffx.89508.1.A1_s_at | 0.235 | CK606172 | Secondary Metabolism | | | Phenylalanine and histidine ammonia- |
| | | | | | | | lyase |
| 907 | Gma.10804.2.A1_s_at | 0.234 | CF921045 | Protein Destination & Storage | | | Molecular chaperone (DnaJ superfamily) |
| 908 | GmaAffx.52337.1.A1_at | 0.234 | CD413673 | No Homology to Known | | | |
| | | | | Proteins | | | |
| 909 | Gma.8988.1.S1_at | 0.234 | BU965547 | No Homology to Known | | | |
| | | | | Proteins | | | |
| 910 | GmaAffx.52391.1.S1_at | 0.233 | AW349563 | Unclassified - Hypothetical | | | |
| | | | | Protein NOT Supported by | | | |
| | | | | cDNA | | | |
| 911 | Gma.4837.1.S1_at | 0.232 | BI969343 | Transporter | | | H+/oligopeptide symporter |
| 912 | Gma.7788.1.A1_at | 0.232 | BU549148 | Unclassified - Hypothetical | | | Aspartyl protease |
| | | | | Protein Supported by cDNA | | | |
| 913 | GmaAffx.92230.1.A1_s_at | 0.23 | CF807955 | Disease & Defense | | | |
| 914 | Gma.13856.1.A1_at | 0.23 | CD408222 | No Homology to Known | | | |
| | | | | Proteins | | | |
| 915 | Gma.5294.1.S1_s_at | 0.23 | BE658252 | Unclassified - Hypothetical | | | |
| | | | | Protein Supported by cDNA | | | |
| 916 | GmaAffx.1242.1.A1_at | 0.229 | BU549839 | Metabolism | | | |
| 917 | Gma.15184.1.S1_at | 0.229 | BM522992 | Transcription | Transcription | Homeodomain | Transcription factor HEX, contains |
| | | | | | Factor | | HOX and HALZ domains |
| 918 | GmaAffx.73554.1.S1_at | 0.228 | BM139469 | Unclassified - Hypothetical | | | |
| | | | | Protein NOT Supported by | | | |
| | | | | cDNA | | | |
| 919 | Gma.17326.1.A1_at | 0.228 | AW568254 | Unclassified - Hypothetical | | | Permease of the major facilitator |
| | | | | Protein NOT Supported by | | | superfamily |
| | | | | cDNA | | | |
| 920 | Gma.6599.1.S1_at | 0.228 | BQ629084 | Cell Structure | | | |
| 921 | GmaAffx.83818.1.S1_at | 0.227 | BU762716 | No Homology to Known | | | |
| | | | | Proteins | | | |
| 922 | GmaAffx.21041.1.A1_at | 0.227 | AW350819 | No Homology to Known | | | |
| | | | | Proteins | | | |
| 923 | GmaAffx.54085.2.S1_at | 0.226 | BQ629483 | No Homology to Known | | | |
| | | | | Proteins | | | |
| 924 | GmaAffx.88017.1.A1_at | 0.226 | BQ452554 | No Homology to Known | | | |
| | | | | Proteins | | | |
| 925 | GmaAffx.87793.2.S1_at | 0.225 | BQ740671 | Signal Transduction | Protein Kinase | | Glycogen synthase kinase-3 |
| 926 | Gma.16528.1.S1_at | 0.225 | CD398706 | Unclassified - Hypothetical | | | |
| | | | | Protein Supported by cDNA | | | |
| 927 | Gma.10536.1.S1_at | 0.225 | BG508300 | No Homology to Known | | | |
| | | | | Proteins | | | |
| 928 | Gma.10830.1.S1_at | 0.225 | BE820822 | Unclassified - Hypothetical | | | |
| | | | | Protein Supported by cDNA | | | |
| 929 | GmaAffx.33121.1.S1_at | 0.224 | BQ611246 | Unclassified - Hypothetical | | | |
| | | | | Protein Supported by cDNA | | | |
| 930 | Gma.8441.1.S1_at | 0.223 | AY113187.2 | Secondary Metabolism | | | Multicopper oxidases |
| 931 | Gma.595.1.S1_at | 0.222 | AB004271.1 | Metabolism | | | |
| 932 | GmaAffx.19467.1.S1_at | 0.22 | BQ453237 | Signal Transduction | Protein Kinase | | Serine/threonine protein kinase |
| 933 | Gma.4981.1.A1_at | 0.22 | B1970256 | No Homology to Known | | | |
| | | | | Proteins | | | |
| 934 | GmaAffx.81585.1.S1_at | 0.22 | BM187887 | Unclassified - Hypothetical | | | Copper chaperone |
| | | | | Protein NOT Supported by | | | |
| | | | | cDNA | | | |
| 935 | Gma.6642.1.A1_at | 0.219 | BE610052 | Transcription | Transcription | Basic Leucine | FOG: Armadillo/beta-catenin-like |
| | | | | | Factor | Zipper (bZIP) | repeats |
| 936 | Gma.17688.1.S1_s_at | 0.219 | CA937360 | Transcription | Transcription | Basic Helix-Loop- | |
| | | | | | Factor | Helix (bHLH) | |
| 937 | GmaAffx.71222.1.S1_at | 0.218 | AW460050 | Metabolism | | | 5′-AMP-activated protein kinase, gamma |
| | | | | | | | subunit |
| 938 | GmaAffx.73943.1.S1_at | 0.218 | BM891115 | Unclassified - Hypothetical | | | |
| | | | | Protein Supported by cDNA | | | |
| 939 | GmaAffx.41230.1.A1_at | 0.218 | BI471609 | Unclassified - Hypothetical | | | |
| | | | | Protein NOT Supported by | | | |
| | | | | cDNA | | | |
| 940 | GmaAffx.92774.1.S1_at | 0.217 | CF808124 | Cell Structure | | | |
| 941 | Gma.3969.1.S1_s_at | 0.217 | AW349494 | Energy | | | |
| 942 | GmaAffx.21978.1.A1_at | 0.217 | CA803009 | No Homology to Known | | | |
| | | | | Proteins | | | |
| 943 | Gma.1195.1.S1_s_at | 0.217 | AI442801 | Metabolism | | | Predicted flavoprotein |
| 944 | GmaAffx.52114.1.S1_at | 0.216 | CA852143 | Metabolism | | | Heme oxygenase |
| 945 | GmaAffx.79390.3.S1_at | 0.216 | BU082743 | Transcription | Transcription | Basic Leucine | FOG: Armadillo/beta-catenin-like |
| | | | | | Factor | Zipper (bZIP) | repeats |
| 946 | GmaAffx.34453.1.S1_at | 0.215 | BU544114 | Unclassified - Hypothetical | | | |
| | | | | Protein Supported by cDNA | | | |
| 947 | Gma.13218.1.A1_s_at | 0.213 | CD391892 | No Homology to Known | | | |
| | | | | Proteins | | | |
| 948 | GmaAffx.61130.1.S1_at | 0.213 | CA799668 | Transporter | | | H+/oligopeptide symporter |
| 949 | GmaAffx.41078.1.S1_at | 0.213 | BI317149 | Metabolism | | | Alcohol dehydrogenase, class III |
| 950 | GmaAffx.49777.I.S1_at | 0.212 | BG047001 | No Homology to Known | | | |
| | | | | Proteins | | | |
| 951 | Gma.7254.1.S1_at | 0.212 | BU546166 | Unclassified - Hypothetical | | | |
| | | | | Protein Supported by cDNA | | | |
| 952 | GmaAffx.53256.1.S1_at | 0.211 | BU545086 | Unclassified - Hypothetical | | | |
| | | | | Protein Supported by cDNA | | | |
| 953 | Gma.11873.1.S1_at | 0.211 | CA8I9977 | Unclassified - Protein with | | | |
| | | | | Unknown Function | | | |
| 954 | Gma.2887.2.S1_x_at | 0.211 | BI701520 | Secondary Metabolism | | | Phenylalanine and histidine ammonia- |
| | | | | | | | lyase |
| 955 | Gma.2376.1.S1_at | 0.209 | CA852095 | Transcription | Transcription | Homeodomain | Transcription factor MEISI and related |
| | | | | | Factor | | HOX domain proteins |
| 956 | GmaAffx.15196.1.S1_at | 0.209 | BU549436 | Metabolism | | | Long-chain acyl-CoA synthetases (AMP- |
| | | | | | | | forming) |
| 957 | Gma.1490.1.S1_at | 0.209 | AB000097.1 | Cell Structure | | | Chitinase |
| 958 | Gma.3332.1.S1_at | 0.209 | BQ298780 | Protein Destination & Storage | | | |
| 959 | GmaArfx.14141.1.A1_at | 0.208 | B1973009 | No Homology to Known | | | |
| | | | | Proteins | | | |
| 960 | GmaAffx.88675.1.A1_at | 0.208 | CD417704 | Unclassified - Hypothetical | | | |
| | | | | Protein NOT Supported by | | | |
| | | | | cDNA | | | |
| 961 | Gma.18006.1.S1_at | 0.207 | CF922923 | Transcription | Transcription | Homeodomain | Transcription factor ME1S1 and related |
| | | | | | Factor | | HOX domain proteins |
| 962 | Gma.57.1.S1_at | 0.206 | AF532624.1 | Unclassified - Hypothetical | | | |
| | | | | Protein NOT Supported by | | | |
| | | | | cDNA | | | |
| 963 | Gma.5711.1.S1_at | 0.206 | B1784811 | Unclassified - Hypothetical | | | |
| | | | | Protein Supported by cDNA | | | |
| 964 | GmaAffx.56715.1.S1_at | 0.206 | BG041616 | Disease & Defense | | | |
| 965 | GmaAffx.42587.1.A1_at | 0.206 | BU544395 | No Homology to Known | | | |
| | | | | Proteins | | | |
| 966 | GmaAffx.91428.1.A1_at | 0.206 | CF807137 | Metabolism | | | |
| 967 | GmaAffx.8999.2.A1_s_at | 0.205 | BI700574 | No Homology to Known | | | |
| | | | | Proteins | | | |
| 968 | GmaAffx.12665.1.A1_at | 0.205 | BE822073 | Metabolism | | | |
| 969 | GmaAffx.43297.1.A1_at | 0.205 | AI442864 | Unclassified - Hypothetical | | | |
| | | | | Protein NOT Supported by | | | |
| | | | | cDNA | | | |
| 970 | Gma.15907.2.S1_at | 0.205 | BE329550 | Signal Transduction | Unclassified | | FOG: Leucine rich repeat |
| 971 | GmaAffx.35656.1.A1_at | 0.204 | BE822348 | No Homology to Known | | | |
| | | | | Proteins | | | |
| 972 | GmaAffx.62509.1.S1_s_at | 0.204 | BE022332 | Transcription | Transcription | MYB | Transcription factor, Myb superfamily |
| | | | | | Factor | | |
| 973 | Gma.4728.1.A1_at | 0.203 | B1968944 | Secondary Metabolism | | | NADH: flavin oxidoreductase/12- |
| | | | | | | | oxophytodienoate reductase |
| 974 | Gma.4746.1.A1_s_at | 0.203 | CD404836 | No Homology to Known | | | |
| | | | | Proteins | | | |
| 975 | GmaAffx.17918.1.S1_at | 0.203 | BG157024 | No Homology to Known | | | |
| | | | | Proteins | | | |
| 976 | Gma.149.1.S1_at | 0.203 | U43839.1 | Metabolism | | | Choline kinase |
| 977 | GmaAffx.48718.1.S1_at | 0.202 | BM528538 | No Homology to Known | | | |
| | | | | Proteins | | | |
| 978 | GmaAffx.64037.1.S1_at | 0.202 | B1321162 | Metabolism | Cytochrome | CYP82F | Cytochrome P450 CYP2 subfamily |
| | | | | | P450 | | |
| 979 | Gma.1929.1.S1_at | 0.201 | BU083650 | Transcription | Transcription | Homeodomain | Transcription factor HEX, contains |
| | | | | | Factor | | HOX and HALZ domains |
| 980 | Gma.2686.1.S1_s_at | 0.201 | B1970455 | Secondary Metabolism | | | |
| 981 | Gma.7549.1.S1_at | 0.201 | BG508637 | Unclassified - Protein with | | | |
| | | | | Unknown Function | | | |
| 982 | Gma.3987.2.S1_a_at | 0.2 | B1971594 | No Homology to Known | | | |
| | | | | Proteins | | | |
| 983 | GmaAffx.91641.1.S1_s_at | 0.2 | CF807366 | Disease & Defense | | | Acyl-CoA synthetase |
| 984 | Gma.3519.1.S1_at | 0.2 | CD403581 | Unclassified - Hypothetical | | | |
| | | | | Protein Supported by cDNA | | | |
| 985 | GmaAffx.11242.1.S1_s_at | 0.2 | AW317461 | Protein Destination & Storage | | | Serine carboxypeptidases |
| 986 | Gma.11522.2.S1_at | 0.199 | BG650872 | Unclassified - Hypothetical | | | |
| | | | | Protein Supported by cDNA | | | |
| 987 | Gma.16196.1.S1_at | 0.199 | AW350888 | Transcription | Transcription | Unclassified | |
| | | | | | Factor | | |
| 988 | GmaAffx.8712.1.S1_s_at | 0.199 | AW349107 | Disease & Defense | | | |
| 989 | GmaAffx.92649.1.S1_at | 0.198 | CF808374 | Metabolism | | | |
| 990 | Gma.7437.1.S1_at | 0.197 | BU547705 | Unclassified - Hypothetical | | | |
| | | | | Protein Supported by cDNA | | | |
| 991 | Gma.4099.1.S1_at | 0.196 | BG651846 | Metabolism | | | Beta-galactosidase |
| 992 | GmaAffx.32700.1.S1_at | 0.196 | BM522940 | No Homology to Known | | | |
| | | | | Proteins | | | |
| 993 | Gma.17662.2.S1_s_at | 0.195 | BQ630190 | Unclassified - Hypothetical | | | |
| | | | | Protein NOT Supported by | | | |
| | | | | cDNA | | | |
| 994 | GmaAffx.55657.1.S1_at | 0.193 | BM885600 | No Homology to Known | | | |
| | | | | Proteins | | | |
| 995 | Gma.3744.1.S1_at | 0.191 | CD417438 | Unclassified - Protein With | | | |
| | | | | Unknown Function | | | |
| 996 | Gma.8966.1.S1_at | 0.19 | BU765374 | Protein Destination & Storage | | | Predicted lipase |
| 997 | Gma.3118.1.S1_s_at | 0.189 | BQ786048 | Disease & Defense | | | Predicted metallothionein |
| 998 | Gma.6498.1.A1_at | 0.189 | BE824396 | Unclassified - Hypothetical | | | |
| | | | | Protein Supported by cDNA | | | |
| 999 | GmaAffx.6635.1.S1_at | 0.189 | CD412718 | Disease & Defense | | | |
| 1000 | GmaAffx.74323.1.S1_at | 0.188 | B1426587 | Unclassified - Hypothetical | | | Endoplasmic reticulum protein EP58. |
| | | | | Protein NOT Supported by | | | contains filamin rod domain and KDEL |
| | | | | cDNA | | | motif |
| 1001 | Gma.15625.1.S1_x_at | 0.188 | U77678.1 | Metabolism | Amino Acid | | Asparagine synthase (glutamine- |
| | | | | | Biosynthesis/ | | hydrolyzing) |
| | | | | | Metabolism | | |
| 1002 | GmaAffx.56301.1.S1_at | 0.188 | BQ610805 | No Homology to Known | | | |
| | | | | Proteins | | | |
| 1003 | GmaAffx.92693.1.S1_at | 0.186 | CF808418 | No Homology to Known | | | |
| | | | | Proteins | | | |
| 1004 | GmaAffx.23292.1.A1_at | 0.185 | B1967795 | No Homology to Known | | | |
| | | | | Proteins | | | |
| 1005 | GmaAffx.295.1.A1_s_at | 0.185 | BG653267 | Protein Destination & Storage | | | |
| 1006 | GmaAffx.27496.2.S1_at | 0.185 | BM523609 | Metabolism | | | |
| 1007 | Gma.12684.1.S1_at | 0.185 | CD394773 | Transcription | Transcription | Zinc Finger | |
| | | | | | Factor | | |
| 1008 | Gma.3744.2.S1_a_at | 0.185 | B1892693 | Unclassified - Protein With | | | |
| | | | | Unknown Function | | | |
| 1009 | Gma.10312.1.S1_at | 0.184 | AW706614 | Unclassified - Hypothetical | | | |
| | | | | Protein NOT Supported by | | | |
| | | | | cDNA | | | |
| 1010 | GmaAffx.81750.2.S1_at | 0.183 | BM568090 | Transcription | Transcription | Homeodomain | Transcription factor MEIS1 and related |
| | | | | | Factor | | HOX domain proteins |
| 1011 | GmaAffx.43809.1.S1_at | 0.183 | AW396565 | No Homology to Known | | | |
| | | | | Proteins | | | |
| 1012 | GmaAffx.68724.1.A1_at | 0.183 | B1970438 | No Homology to Known | | | |
| | | | | Proteins | | | |
| 1013 | Gma.3951.1.S1_at | 0.182 | BE659337 | Unclassified - Hypothetical | | | |
| | | | | Protein NOT Supported by | | | |
| | | | | cDNA | | | |
| 1014 | Gma.3165.1.S1_at | 0.181 | CD406755 | Transcription | Transcription | Homeodomain | Transcription factor HEX, contains |
| | | | | | Factor | | HOX and HALZ domains |
| 1015 | GmaAffx.14279.1.S1_at | 0.18 | BM887020 | Metabolism | | | |
| 1016 | Gma.6481.1.S1_at | 0.178 | CD416703 | Unclassified - Hypothetical | | | |
| | | | | Protein Supported by cDNA | | | |
| 1017 | Gma.15625.1.S1_a_at | 0.178 | U77678.1 | Metabolism | Amino Acid | | Asparagine synthase (glutamine- |
| | | | | | Biosynthesis/ | | hydrolyzing) |
| | | | | | Metabolism | | |
| 1018 | GmaAffx.70008.1.S1_at | 0.178 | BU762337 | Transcription | Transcription | MYB | Transcription factor, Myb superfamily |
| | | | | | Factor | | |
| 1019 | GmaAffx.26868.1.S1_at | 0.178 | BF325443 | Unclassified - Hypothetical | | | Tomosyn and related SNARE- |
| | | | | Protein NOT Supported by | | | interacting proteins |
| | | | | cDNA | | | |
| 1020 | GmaAffx.21924.1.S1_at | 0.177 | BQ611867 | Disease & Defense | | | |
| 1021 | Gma.9957.1.S1_at | 0.177 | BE820632 | Metabolism | | | UDP-glucuronosyl and UDP-glucosyl |
| | | | | | | | transferase |
| 1022 | Gma.17921.2.S1_s_at | 0.177 | BM085020 | Metabolism | Sucrose | | Glycosyltransferase |
| | | | | | Biosynthesis | | |
| 1023 | GmaAffx.40453.1.S1_at | 0.176 | BE658683 | No Homology to Known | | | |
| | | | | Proteins | | | |
| 1024 | GmaAffx.71842.1.S1_at | 0.176 | BE555317 | Metabolism | | | Possible pfkB family carbohydrate |
| | | | | | | | kinase |
| 1025 | GmaAffx.30792.1.A1_at | 0.176 | BU544815 | Unclassified - Hypothetical | | | |
| | | | | Protein Supported by cDNA | | | |
| 1026 | Gma.6192.1.S1_s_at | 0.175 | AW396773 | Metabolism | | | |
| 1027 | GmaAffx.46837.1.S1_at | 0.175 | BU550919 | Secondary Metabolism | | | Multicopper oxidases |
| 1028 | Gma.15534.1.S1_at | 0.175 | U50081.1 | Metabolism | | | |
| 1029 | GmaAffx.45036.1.S1_at | 0.175 | BF595470 | No Homology to Known | | | |
| | | | | Proteins | | | |
| 1030 | Gma.8490.1.S1_at | 0.175 | AB030492.1 | Metabolism | | | Protein involved in thiamine |
| | | | | | | | biosynthesis and DNA damage tolerance |
| 1031 | Gma.7422.1.S1_at | 0.174 | BQ612005 | Transporter | | | |
| 1032 | GmaAffx.79390.1.S1_at | 0.174 | BU550458 | Transcription | Transcription | Basic Leucine | FOG: Armadillo/beta-catenin-like |
| | | | | | Factor | Zipper (bZIP) | repeats |
| 1033 | Gma.1137.2.S1_x_at | 0.173 | CA852403 | Metabolism | | | |
| 1034 | Gma.6081.1.S1_at | 0.173 | B1967474 | Disease & Defense | | | Predicted chitinase |
| 1035 | GmaAffx.71308.1.S1_at | 0.173 | BF595112 | Transcription | Transcription | Heat Shock | Heat shock transcription factor |
| | | | | | Factor | | |
| 1036 | Gma.3522.1.S1_at | 0.173 | BM140209 | Unclassified - Hypothetical | | | |
| | | | | Protein Supported by cDNA | | | |
| 1037 | Gma.2801.1.S1_at | 0.173 | B1785739 | Cell Structure | | | Beta-glucosidase, lactase |
| | | | | | | | phlorizinhydrolase, and related proteins |
| 1038 | Gma.8370.1.S1_at | 0.172 | BQ454087 | Signal Transduction | Protein | | Serine/threonine protein phosphatase |
| | | | | | Phosphatase | | |
| 1039 | GmaAffx.89259.1.S1_s_at | 0.172 | CK605646 | Metabolism | | | |
| 1040 | GmaAffx.93436.1.A1_s_at | 0.17 | CF809161 | Transcription | Transcription | AP2/EREBP | |
| | | | | | Factor | | |
| 1041 | Gma.8704.1.S1_at | 0.17 | AW311022 | Unclassified - Hypothetical | | | FOG: TPR repeat |
| | | | | Protein NOT Supported by | | | |
| | | | | cDNA | | | |
| 1041 | Gma.8704.1.S1_at | 0.17 | AW311022 | Unclassified - Hypothetical | | | Thioredoxin |
| | | | | Protein NOT Supported by | | | |
| | | | | cDNA | | | |
| 1042 | GmaAffx.90677.1.S1_at | 0.17 | CF806402 | Unclassified - Hypothetical | | | DNA helicase P1F1/RRM3 |
| | | | | Protein NOT Supported by | | | |
| | | | | cDNA | | | |
| 1043 | GmaAffx.47230.1.S1_at | 0.169 | CA820051 | Transporter | | | Amino acid transporters |
| 1044 | Gma.16913.1.S1_s_at | 0.169 | B1426712 | Unclassified - Protein with | | | |
| | | | | Unknown Function | | | |
| 1045 | Gma.3000.2.S1_a_at | 0.168 | BM270844 | No Homology to Known | | | |
| | | | | Proteins | | | |
| 1046 | Gma.1394.1.S1_at | 0.167 | BG839300 | Metabolism | | | |
| 1047 | Gma.4120.2.S1_at | 0.167 | B1700246 | Unclassified - Protein with | | | |
| | | | | Unknown Function | | | |
| 1048 | Gma.3987.1.S1_at | 0.165 | BQ453408 | Unclassified - Hypothetical | | | Sodium sulfate symporter and related |
| | | | | Protein NOT Supported by | | | arsenite permeases |
| | | | | cDNA | | | |
| 1049 | GmaAffx.62509.2.S1_at | 0.164 | BE021475 | Transcription | Transcription | MYB | Transcription factor, Myb superfamily |
| | | | | | Factor | | |
| 1050 | GmaAffx.41917.1.S1_at | 0.164 | BE658926 | Disease & Defense | | | |
| 1051 | Gma.4095.1.S1_at | 0.164 | AW350037 | Unclassified - Hypothetical | | | |
| | | | | Protein Supported by cDNA | | | |
| 1052 | Gma.9036.1.S1_at | 0.163 | AW309501 | No Homology to Known | | | |
| | | | | Proteins | | | |
| 1053 | GmaAffx.55959.1.S1_at | 0.163 | BQ629429 | Unclassified - Hypothetical | | | |
| | | | | Protein NOT Supported by | | | |
| | | | | cDNA | | | |
| 1054 | Gma.4394.1.S1_a_at | 0.163 | CD409273 | Transcription | Transcription | Homeodomain | Transcription factor HEX, contains |
| | | | | | Factor | | HOX and HALZ domains |
| 1055 | Gma.3575.1.S1_at | 0.162 | BQ611599 | Cell Structure | | | |
| 1056 | Gma.1394.2.S1_at | 0.162 | B1470370 | Metabolism | | | |
| 1057 | Gma.5961.1.S1_at | 0.162 | AB052786.1 | Transporter | | | H+/oligopeptide symporter |
| 1058 | GmaAffx.39765.1.S1_s_at | 0.161 | BQ785586 | No Homology to Known | | | |
| | | | | Proteins | | | |
| 1059 | Gma.4479.1.S1_at | 0.161 | CA936529 | Unclassified - Hypothetical | | | |
| | | | | Protein Supported by cDNA | | | |
| 1060 | GmaAffx.89772.14.A1_s_at | 0.161 | CK606614 | Disease & Defense | | | |
| 1061 | Gma.10969.2.S1_at | 0.16 | CD402329 | Unclassified - Hypothetical | | | Predicted lipase |
| | | | | Protein NOT Supported by | | | |
| | | | | cDNA | | | |
| 1062 | GmaAffx.11815.1.S1_at | 0.16 | AW311637 | Metabolism | | | |
| 1063 | Gma.6192.1.S1_at | 0.159 | AW396773 | Metabolism | | | |
| 1064 | GmaAffx.77758.1.S1_x_at | 0.158 | B1944003 | No Homology to Known | | | |
| | | | | Proteins | | | |
| 1065 | Gma.12227.1.S1_at | 0.157 | BG511671 | Unclassified - Hypothetical | | | |
| | | | | Protein NOT Supported by | | | |
| | | | | cDNA | | | |
| 1066 | GmaAffx.15119.1.A1_at | 0.157 | BU544528 | Metabolism | | | Possible pfkB family carbohydrate |
| | | | | | | | kinase |
| 1067 | Gma.13150.1.A1_at | 0.157 | CD411577 | Unclassified - Protein with | Hormone | | |
| | | | | Unknown Function | Induced | | |
| 1068 | Gma.15996.1.S1_at | 0.156 | BQ454193 | Unclassified - Hypothetical | | | |
| | | | | Protein Supported by cDNA | | | |
| 1069 | GmaAffx.90812.1.S1_s_at | 0.155 | CF806537 | Disease & Defense | | | |
| 1070 | Gma.18006.1.A1_at | 0.155 | AI496486 | Transcription | Transcription | Homeodomain | Transcription factor MEIS1 and related |
| | | | | | Factor | | HOX domain proteins |
| 1071 | Gma.5863.2.S1_s_at | 0.155 | AW350762 | Secondary Metabolism | | | |
| 1072 | Gma.17739.1.S1_at | 0.155 | AW310535 | Unclassified - Hypothetical | | | |
| | | | | Protein Supported by cDNA | | | |
| 1073 | GmaAffx.81584.1.S1_at | 0.154 | B1426969 | Disease & Defense | | | |
| 1074 | GmaAffx.71308.2.A1_at | 0.153 | AW569888 | No Homology to Known | | | |
| | | | | Proteins | | | |
| 1075 | GmaAffx.5531.1.S1_at | 0.153 | BQ740711 | Cell Structure | | | |
| 1076 | Gma.10621.1.S1_at | 0.153 | AY004243.1 | Unclassified - Hypothetical | | | |
| | | | | Protein Supported by cDNA | | | |
| 1077 | Gma.14606.1.A1_at | 0.153 | BE821928 | Transporter | | | |
| 1078 | Gma.11028.1.S1_s_at | 0.152 | BE823084 | Metabolism | | | Glyoxylate/hydroxypyruvate reductase |
| | | | | | | | (D-isomer-specific 2-hydroxy acid |
| | | | | | | | dehydrogenase superfamily) |
| 1079 | Gma.8489.1.S1_s_at | 0.152 | AB030493.1 | Metabolism | | | Protein involved in thiamine |
| | | | | | | | biosynthesis and DNA damage tolerance |
| 1080 | Gma.4874.1.A1_at | 0.152 | AI938293 | No Homology to Known | | | |
| | | | | Proteins | | | |
| 1081 | Gma.5331.1.S1_at | 0.152 | BI892702 | Transcription | Transcription | NAC Domain | |
| | | | | | Factor | | |
| 1082 | GmaAffx.70005.1.S1_s_at | 0.15 | CA936335 | Secondary Metabolism | | | Iron/ascorbate family oxidoreductases |
| 1083 | GmaAffx.27331.1.S1_at | 0.15 | BM520297 | Unclassified - Hypothetical | | | |
| | | | | Protein NOT Supported by | | | |
| | | | | cDNA | | | |
| 1084 | Gma.13062.1.S1_at | 0.149 | CD394315 | Metabolism | | | |
| 1085 | Gma.4150.2.S1_s_at | 0.149 | AW350622 | Unclassified - Hypothetical | | | |
| | | | | Protein Supported by cDNA | | | |
| 1086 | Gma.6129.2.S1_s_at | 0.148 | AW186006 | Protein Synthesis | | | Translation initiation factor 5A (eIF-5A) |
| 1087 | GmaAffx.11172.1.A1_at | 0.148 | CD404556 | No Homology to Known | | | |
| | | | | Proteins | | | |
| 1088 | GmaAffx.91555.1.A1_s_at | 0.148 | CF807280 | Metabolism | | | |
| 1089 | Gma.6999.3.S1_s_at | 0.147 | CF921432 | Disease & Defense | | | |
| 1090 | Gma.16842.1.A1_at | 0.147 | CD392652 | No Homology to Known | | | |
| | | | | Proteins | | | |
| 1091 | Gma.3604.1.S1_at | 0.147 | AW349604 | Cell Structure | | | O-methyltransferase |
| 1092 | GmaAffx.1785.1.S1_at | 0.146 | BF325331 | Transcription | Transcription | Homeodomain | Transcription factor HEX, contains |
| | | | | | Factor | | HOX and HALZ domains |
| 1093 | GmaAffx.93328.1.S1_s_at | 0.145 | CF809053 | Secondary Metabolism | | | |
| 1094 | GmaAffx.91205.1.S1_at | 0.144 | CF807869 | Metabolism | | | Glyoxylate/hydroxypyruvate reductase |
| | | | | | | | (D-isomer-specific 2-hydroxy acid |
| | | | | | | | dehydrogenase superfamily) |
| 1095 | GmaAffx.52963.1.S1_at | 0.144 | AW310673 | Unclassified - Protein with | | | |
| | | | | Unknown Function | | | |
| 1096 | GmaAffx.59470.1.S1_at | 0.143 | B1469872 | Secondary Metabolism | | | Phenylalanine and histidine ammonia- |
| | | | | | | | lyase |
| 1097 | GmaAffx.24992.1.S1_at | 0.143 | BU083490 | Protein Destination & Storage | | | Aspartyl protease |
| 1098 | GmaAffx.72700.1.S1_at | 0.143 | AW349825 | Unclassified - Hypothetical | | | |
| | | | | Protein NOT Supported by | | | |
| | | | | cDNA | | | |
| 1099 | Gma.11026.2.S1_s_at | 0.143 | BQ080041 | Disease & Defense | | | |
| 1100 | Gma.6643.1.A1_at | 0.143 | BE823205 | Secondary Metabolism | | | Sulfotransferase |
| 1101 | Gma.8318.1.A1_at | 0.143 | BU550469 | No Homology to Known | | | |
| | | | | Proteins | | | |
| 1102 | Gma.11026.1.S1_at | 0.143 | AW756869 | Disease & Defense | | | |
| 1103 | GmaAffx.4968.1.S1_at | 0.142 | BI967691 | Unclassified - Hypothetical | | | |
| | | | | Protein Supported by cDNA | | | |
| 1104 | GmaAffx.14279.2.A1_at | 0.142 | BM095037 | Metabolism | | | |
| 1105 | Gma.3799.2.S1_s_at | 0.142 | AW348702 | Unclassified - Protein with | | | |
| | | | | Unknown Function | | | |
| 1106 | GmaAffx.60479.1.S1_at | 0.141 | BG726023 | Transcription | Transcription | Homeodomain | Transcription factor ME1S1 and related |
| | | | | | Factor | | HOX domain proteins |
| 1107 | Gma.2842.1.S1_at | 0.141 | BG511652 | Unclassified - Protein with | | | Predicted Yippee-type zinc-binding |
| | | | | Unknown Function | | | protein |
| 1108 | GmaAffx.34657.1.A1_s_at | 0.14 | AW351012 | No Homology to Known | | | |
| | | | | Proteins | | | |
| 1109 | Gma.5760.2.S1_at | 0.14 | BQ298381 | Disease & Defense | | | |
| 1110 | GmaAffx.87433.1.S1_at | 0.14 | B1701509 | Unclassified - Hypothetical | | | |
| | | | | Protein Supported by cDNA | | | |
| 1111 | Gma.8187.1.S1_a_at | 0.139 | BU549133 | Signal Transduction | Protein | | Serine/threonine protein phosphatase |
| | | | | | Phosphatase | | |
| 1112 | Gma.17686.1.S1_at | 0.138 | BF597378 | Metabolism | Cytochrome | CYP76C | Cytochrome P450 CYP2 subfamily |
| | | | | | P450 | | |
| 1113 | Gma.6278.1.S1_at | 0.137 | BQ612647 | Unclassified - Hypothetical | | | |
| | | | | Protein Supported by cDNA | | | |
| 1114 | Gma.6643.2.S1_at | 0.137 | BE023121 | Secondary Metabolism | | | Sulfotransferase |
| 1115 | Gma.5867.1.A1_s_at | 0.136 | B1316163 | Metabolism | | | |
| 1116 | GmaAffx.81750.1.S1_at | 0.135 | AW306336 | No Homology to Known | | | |
| | | | | Proteins | | | |
| 1117 | GmaAffx.9944.1.S1_at | 0.135 | BM567882 | Cell Structure | | | |
| 1118 | Gma.5863.1.S1_at | 0.134 | AJ003245.1 | Secondary Metabolism | | | |
| 1119 | Gma.10969.1.S1_a_at | 0.133 | U36191.1 | Metabolism | | | |
| 1120 | GmaAffx.3711.1.A1_at | 0.133 | BE822116 | Unclassified - Hypothetical | | | |
| | | | | Protein NOT Supported by | | | |
| | | | | cDNA | | | |
| 1121 | GmaAffx.61593.1.S1_at | 0.133 | BU765478 | Unclassified - Hypothetical | | | Sodium sulfate symporter and related |
| | | | | Protein NOT Supported by | | | arsenite permeases |
| | | | | cDNA | | | |
| 1122 | Gma.5469.1.S1_at | 0.132 | AW349200 | No Homology to Known | | | |
| | | | | Proteins | | | |
| 1123 | Gma.17446.1.A1_at | 0.132 | AI461103 | Unclassified - Protein with | | | |
| | | | | Unknown Function | | | |
| 1124 | GmaAffx.17961.1.A1_at | 0.132 | BE657601 | Protein Destination & Storage | | | Molecular chaperones HSP70/HSC70, |
| | | | | | | | HSP70 superfamily |
| 1125 | Gma.3776.1.S1_at | 0.131 | AW780736 | Metabolism | | | Glutaredoxin and related proteins |
| 1126 | Gma.14471.1.S1_s_at | 0.131 | CF921241 | No Homology to Known | | | |
| | | | | Proteins | | | |
| 1127 | Gma.6129.2.S1_at | 0.131 | AW186006 | Protein Synthesis | | | Translation initiation factor 5A (eIF-5A) |
| 1128 | GmaAffx.47806.1.A1_at | 0.13 | BG508458 | No Homology to Known | | | |
| | | | | Proteins | | | |
| 1129 | Gma.12094.1.A1_at | 0.13 | CD395088 | Unclassified - Hypothetical | | | |
| | | | | Protein Supported by cDNA | | | |
| 1130 | Gma.3578.1.S1_at | 0.129 | AW156522 | Disease & Defense | | | Acyl-CoA synthetase |
| 1131 | Gma.10896.3.A1_at | 0.129 | BI316837 | Metabolism | | | Aldehyde dehydrogenase |
| 1132 | GmaAffx.90677.1.S1_s_at | 0.128 | CF806402 | Unclassified - Hypothetical | | | DNA helicase PIF1/RRM3 |
| | | | | Protein NOT Supported by | | | |
| | | | | cDNA | | | |
| 1133 | Gma.10969.3.S1_x_at | 0.128 | CD396552 | Metabolism | | | |
| 1134 | Gma.4928.1.S1_a_at | 0.127 | AF002259.1 | Disease & Defense | | | Acyl-CoA synthetase |
| 1135 | GmaAffx.81362.1.S1_at | 0.126 | BF324988 | Cell Structure | | | |
| 1136 | GmaAffx.74256.1.S1_at | 0.126 | BE057293 | No Homology to Known | | | |
| | | | | Proteins | | | |
| 1137 | GmaAffx.91312.1.S1_s_at | 0.126 | CF806002 | Transcription | Transcription | Homeodomain | Transcription factor HEX, contains |
| | | | | | Factor | | HOX and HALZ domains |
| 1138 | Gma.2252.1.S1_at | 0.126 | CD406598 | Unclassified - Hypothetical | | | |
| | | | | Protein Supported by cDNA | | | |
| 1139 | GmaAffx.10204.1.S1_at | 0.126 | BQ298213 | Unclassified - Hypothetical | | | |
| | | | | Protein Supported by cDNA | | | |
| 1140 | Gma.3578.1.S1_s_at | 0.126 | AW156522 | Disease & Defense | | | Acyl-CoA synthetase |
| 1141 | Gma.1527.1.S1_x_at | 0.125 | B1968185 | Secondary Metabolism | | | Flavonol reductase/cinnamoyl-CoA |
| | | | | | | | reductase |
| 1142 | GmaAffx.93575.1.S1_s_at | 0.124 | CF808812 | No Homology to Known | | | |
| | | | | Proteins | | | |
| 1143 | GmaAffx.82218.1.S1_at | 0.124 | BI702052 | Secondary Metabolism | | | Iron/ascorbate family oxidoreductases |
| 1144 | Gma.4266.1.S1_at | 0.121 | B1972260 | No Homology to Known | | | |
| | | | | Proteins | | | |
| 1145 | Gma.9647.1.S1_at | 0.121 | BE820997 | Cell Structure | | | O-methyltransferase |
| 1146 | Gma.15.1.S1_at | 0.119 | U66316.1 | Unclassified - Protein with | | | |
| | | | | Unknown Function | | | |
| 1147 | Gma.6999.1.S1_s_at | 0.119 | AF529303.1 | Disease & Defense | | | |
| 1148 | GmaAffx.1301.78.S1_s_at | 0.119 | AW394497 | No Homology to Known | | | |
| | | | | Proteins | | | |
| 1149 | Gma.10827.3.S1_at | 0.119 | AW758966 | Secondary Metabolism | | | Iron/ascorbate family oxidoreductases |
| 1150 | Gma.3914.1.S1_at | 0.118 | BI702206 | Unclassified - Hypothetical | | | |
| | | | | Protein Supported by cDNA | | | |
| 1151 | Gma.17877.1.S1_at | 0.118 | BQ612471 | Unclassified - Protein with | | | |
| | | | | Unknown Function | | | |
| 1152 | Gma.320.1.S1_at | 0.117 | AF019116.1 | Disease & Defense | | | |
| 1153 | Gma.15523.1.S1_s_at | 0.116 | BE475550 | Cell Structure | | | |
| 1154 | Gma.16566.1.S1_at | 0.116 | CD400974 | Metabolism | | | Fumarase |
| 1155 | GmaAffx.90600.1.S1_at | 0.116 | CF806325 | Metabolism | | | |
| 1156 | Gma.2986.1.S1_at | 0.115 | BM271324 | Metabolism | | | 5′-AMP-activated protein kinase, gamma |
| | | | | | | | subunit |
| 1157 | GmaAffx.82000.1.S1_at | 0.115 | B1094895 | Unclassified - Hypothetical | | | |
| | | | | Protein Supported by cDNA | | | |
| 1158 | Gma.1269.1.S1_at | 0.115 | AW309671 | Metabolism | | | Flavonol reductase/cinnamoyl-CoA |
| | | | | | | | reductase |
| 1159 | Gma.14338.1.A1_at | 0.114 | CF923111 | Protein Destination & Storage | Storage Protein | | |
| 1160 | GmaAffx.73698.1.S1_at | 0.114 | BU964780 | Metabolism | | | 5′-AMP-activated protein kinase, gamma |
| | | | | | | | subunit |
| 1161 | GmaAffx.87793.1.S1_at | 0.114 | B1941782 | Unclassified - Hypothetical | | | |
| | | | | Protein NOT Supported by | | | |
| | | | | cDNA | | | |
| 1162 | Gma.6999.2.S1_s_at | 0.113 | X60043.1 | Disease & Defense | | | |
| 1163 | Gma.9638.1.A1_at | 0.113 | CA936403 | Disease & Defense | | | |
| 1164 | GmaAffx.34975.1.S1_at | 0.112 | BU547817 | Unclassified - Hypothetical | | | |
| | | | | Protein Supported by cDNA | | | |
| 1165 | GmaAffx.478.1.S1_at | 0.112 | BE822248 | Transcription | Transcription | Homeodomain | Transcription factor ME1S1 and related |
| | | | | | Factor | | HOX domain proteins |
| 1166 | Gma.2086.1.S1_at | 0.112 | AF035671.1 | Metabolism | Amino Acid | | Arginase family protein |
| | | | | | Biosynthesis/ | | |
| | | | | | Metabolism | | |
| 1167 | Gma.1661.1.S1_at | 0.111 | BE821382 | Metabolism | | | |
| 1168 | Gma.2133.1.S1_at | 0.11 | AW310071 | Unclassified - Protein with | | | |
| | | | | Unknown Function | | | |
| 1169 | GmaAffx.19541.1.S1_s_at | 0.109 | BQ612990 | No Homology to Known | | | |
| | | | | Proteins | | | |
| 1170 | GmaAffx.92927.1.S1_at | 0.109 | CF805992 | No Homology to Known | | | |
| | | | | Proteins | | | |
| 1171 | GmaAffx.87417.1.S1_at | 0.109 | BM568072 | Metabolism | | | |
| 1172 | GmaAffx.90600.1.S1_s_at | 0.108 | CF806325 | Metabolism | | | |
| 1173 | Gma.9740.1.S1_at | 0.108 | BQ081058 | Unclassified - Hypothetical | | | |
| | | | | Protein Supported by cDNA | | | |
| 1174 | Gma.15036.2.A1_at | 0.107 | CD411972 | Unclassified - Protein with | | | Predicted membrane protein |
| | | | | Unknown Function | | | |
| 1175 | Gma.4120.1.A1_at | 0.106 | AW310714 | Unclassified - Protein with | | | |
| | | | | Unknown Function | | | |
| 1176 | GmaAffx.487.1.S1_at | 0.106 | BG511495 | Transporter | | | Predicted transporter (major facilitator |
| | | | | | | | superfamily) |
| 1177 | Gma.10227.1.A1_at | 0.106 | BE020065 | Metabolism | | | Flavin-containing monooxygenase |
| 1178 | Gma.15568.1.S1_at | 0.103 | AI442650 | Disease & Defense | | | |
| 1179 | GmaAffx.30665.1.A1_at | 0.103 | BQ453743 | Signal Transduction | Protein Kinase | | Serine/threonine protein kinase |
| 1180 | GmaAffx.87670.1.S1_at | 0.103 | BQ452640 | Unclassified - Hypothetical | | | |
| | | | | Protein NOT Supported by | | | |
| | | | | cDNA | | | |
| 1181 | GmaAffx.76276.1.S1_at | 0.103 | BE331452 | Transporter | | | Copper chaperone |
| 1182 | Gma.4687.1.S1_at | 0.102 | AW309716 | Secondary Metabolism | | | |
| 1183 | GmaAffx.92479.1.S1_s_at | 0.101 | CF808204 | No Homology to Known | | | |
| | | | | Proteins | | | |
| 1184 | GmaAffx.34449.1.S1_at | 0.101 | AW164447 | No Homology to Known | | | |
| | | | | Proteins | | | |
| 1185 | Gma.2912.1.S1_s_at | 0.101 | AF117884.1 | Protein Destination & Storage | Storage Protein | | Uncharacterized conserved protein |
| 1186 | Gma.11028.2.S1_s_at | 0.101 | CA782711 | Metabolism | | | Glyoxylate/hydroxypyruvate reductase |
| | | | | | | | (D-isomer-specific 2-hydroxy acid |
| | | | | | | | dehydrogenase superfamily) |
| 1187 | GmaAffx.11897.1.S1_at | 0.1 | BE819868 | Transporter | | | Permease of the major facilitator |
| | | | | | | | superfamily |
| 1188 | Gma.11166.1.S1_s_at | 0.0999 | AW705829 | Metabolism | | | |
| 1189 | GmaAffx.9911.1.S1_at | 0.0948 | BU547808 | Unclassified - Hypothetical | | | Tomosyn and related SNARE- |
| | | | | Protein NOT Supported by | | | interacting proteins |
| | | | | cDNA | | | |
| 1190 | Gma.3774.2.S1_at | 0.0948 | CD393365 | Energy | | | Thioredoxin, nucleoredoxin and related |
| | | | | | | | proteins |
| 1191 | GmaAffx.83880.1.A1_at | 0.0946 | BE822640 | Disease & Defense | | | |
| 1192 | Gma.11166.1.S1_x_at | 0.0946 | AW705829 | Metabolism | | | |
| 1193 | Gma.1445.1.S1_at | 0.0927 | BQ454241 | Metabolism | | | Flavonol reductase/cinnamoyl-CoA |
| | | | | | | | reductase |
| 1194 | GmaAffx.26516.1.S1_at | 0.0916 | AW433266 | Cell Structure | | | |
| 1195 | GmaAffx.70435.2.S1_at | 0.0912 | AW705853 | Transcription | Transcription | Basic Helix-Loop- | |
| | | | | | Factor | Helix (bHLH) | |
| 1196 | Gma.13854.1.S1_at | 0.0911 | CD408166 | Metabolism | | | |
| 1197 | GmaAffx.93591.1.S1_s_at | 0.0905 | CF809316 | Metabolism | | | |
| 1198 | Gma.5622.1.S1_at | 0.0895 | BG363493 | No Homology to Known | | | |
| | | | | Proteins | | | |
| 1199 | GmaAffx.51464.1.S1_at | 0.089 | BQ628397 | No Homology to Known | | | |
| | | | | Proteins | | | |
| 1200 | GmaAffx.51444.1.S1_at | 0.0883 | BM178639 | Secondary Metabolism | | | Multicopper oxidases |
| 1201 | GmaAffx.74899.1.S1_at | 0.0855 | CA852377 | No Homology to Known | | | |
| | | | | Proteins | | | |
| 1202 | GmaAffx.24413.1.A1_at | 0.0849 | BU548368 | Metabolism | | | 5′-AMP-activated protein kinase, gamma |
| | | | | | | | subunit |
| 1203 | GmaAffx.3369.2.A1_at | 0.0845 | BU550316 | Cell Structure | | | |
| 1204 | Gma.17985.2.S1_at | 0.083 | BE802263 | Metabolism | | | |
| 1205 | Gma.15996.1.S1_x_at | 0.0827 | BQ454193 | Unclassified - Hypothetical | | | |
| | | | | Protein Supported by cDNA | | | |
| 1206 | Gma.9947.1.S1_at | 0.0812 | BF324269 | Unclassified - Hypothetical | | | |
| | | | | Protein Supported by cDNA | | | |
| 1207 | GmaAffx.90395.1.S1_s_at | 0.0789 | CF806120 | Unclassified - Protein with | | | |
| | | | | Unknown Function | | | |
| 1208 | Gma.2912.1.S1_at | 0.0785 | AF117884.1 | Protein Destination & Storage | Storage Protein | | Uncharacterized conserved protein |
| 1209 | Gma.4610.1.S1_s_at | 0.073 | BM522856 | No Homology to Known | | | |
| | | | | Proteins | | | |
| 1210 | Gma.79.1.S1_x_at | 0.0729 | S44202.1 | Unclassified - Protein with | | | |
| | | | | Unknown Function | | | |
| 1211 | Gma.1243.1.S1_at | 0.0725 | AW311027 | Unclassified - Hypothetical | | | |
| | | | | Protein Supported by cDNA | | | |
| 1212 | Gma.5947.1.S1_s_at | 0.0722 | BQ610509 | Disease & Defense | | | |
| 1213 | Gma.9010.1.S1_at | 0.0719 | CF921194 | Disease & Defense | | | |
| 1214 | Gma.16947.1.S1_at | 0.0696 | BG155375 | Unclassified - Hypothetical | | | |
| | | | | Protein Supported by cDNA | | | |
| 1215 | Gma.10216.1.S1_at | 0.0693 | B1969369 | Metabolism | | | Hydroxyindole-O-methyltransferase and |
| | | | | | | | related SAM-dependent |
| | | | | | | | methyltransferases |
| 1216 | GmaAffx.89407.1.A1_s_at | 0.0665 | CK606273 | Transporter | | | |
| 1217 | Gma.11211.1.S1_s_at | 0.0639 | AI442578 | No Homology to Known | | | |
| | | | | Proteins | | | |
| 1218 | Gma.17840.1.S1_at | 0.0614 | BE821995 | No Homology to Known | | | |
| | | | | Proteins | | | |
| 1219 | Gma.5611.1.S1_at | 0.0612 | AW309826 | Metabolism | Cytochrome | CYP81D | Cytochrome P450 CYP2 subfamily |
| | | | | | P450 | | |
| 1220 | Gma.79.1.S1_s_at | 0.0565 | S44202.1 | Unclassified - Protein with | | | |
| | | | | Unknown Function | | | |
| 1221 | Gma.4401.2.S1_a_at | 0.0559 | B1970725 | Disease & Defense | | | |
| 1222 | Gma.3774.1.S1_at | 0.0541 | BU550698 | Energy | | | Thioredoxin, nucleoredoxin and related |
| | | | | | | | proteins |
| 1223 | Gma.8425.1.S1_s_at | 0.0512 | D83968.1 | Metabolism | Cytochrome | CYP93D | Cytochrome P450 CYP2 subfamily |
| | | | | | P450 | | |
| 1224 | GmaAffx.10582.1.S1_at | 0.0508 | BM523687 | Transcription | Transcription | CCAAT | CCAAT-binding factor, subunit A |
| | | | | | Factor | | (HAP3) |
| 1225 | Gma.4796.1.A1_at | 0.0486 | AW102323 | Unclassified - Hypothetical | | | Uncharacterized conserved protein |
| | | | | Protein NOT Supported by | | | |
| | | | | cDNA | | | |
| 1226 | Gma.176.1.S1_s_at | 0.0411 | AF004810.1 | Protein Destination & Storage | Storage Protein | | |
| 1227 | Gma.15307.1.S1_at | 0.0401 | U64866.1 | Disease & Defense | | | |
| 1228 | Gma.3774.2.S1_a_at | 0.0399 | CD393365 | Energy | | | Thioredoxin, nucleoredoxin and related |
| | | | | | | | proteins |
| 1229 | Gma.1527.1.S1_a_at | 0.0379 | B1968185 | Secondary Metabolism | | | Flavonol reductase/cinnamoyl-CoA |
| | | | | | | | reductase |
| 1230 | GmaAffx.85382.1.S1_s_at | 0.0377 | B1945154 | No Homology to Known | | | |
| | | | | Proteins | | | |
| 1231 | Gma.1314.1.S1_at | 0.0357 | U63726.1 | Metabolism | | | Gamma-glutamyl hydrolase |
| 1232 | Gma.1527.2.S1_at | 0.029 | BE658017 | Secondary Metabolism | | | Flavonol reductase/cinnamoyl-CoA |
| | | | | | | | reductase |
| 1233 | GmaAffx.68621.1.A1_at | 0.0274 | BI893889 | Unclassified - Protein with | Stress Induced | | |
| | | | | Unknown Function | | | |
| 1234 | Gma.17985.1.S1_at | 0.0204 | CD416450 | Metabolism | | | |

TABLE 14

| | | | Soybean_Gen | | | | |
|---|---|---|---|---|---|---|---|
| ID | Gene Name | Fold Change | Bank_Acc | Functional Category | Sub-Family | Gene Family | Additional Description |
| 1 | Gma.12356.1.A1_at | 2.882 | CD397798 | Unclassified - Hypothetical Protein NOT | | | |
| | | | | Supported by cDNA | | | |
| 2 | GmaAffx.13179.1.S1_at | 2.769 | CD402274 | Unclassified - Hypothetical Protein | | | |
| | | | | NOT Supported by cDNA | | | |
| 3 | Gma.2194.1.S1_at | 2.524 | BE657352 | Unclassified - Hypothetical Protein | | | |
| | | | | Supported by cDNA | | | |
| 4 | GmaAffx.30420.1.S1_at | 2.207 | AW704927 | Transporter | | | |
| 5 | Gma.964.1.A1_at | 2.128 | BE824146 | Metabolism | | | Tyrosine aminotransferase |
| 6 | GmaAffx.87806.1.S1_at | 2.032 | BG882829 | Unclassified - Hypothetical Protein | | | |
| | | | | NOT Supported by cDNA | | | |
| 7 | Gma.10787.2.S1_a_at | 0.5 | BE023083 | Transcription | Transcription Factor | Squamosa | |
| 8 | GmaAffx.84721.1.S1_at | 0.5 | BQ297863 | Metabolism | | | Hydroxymethylglutaryl-CoA lyase |
| 9 | GmaAffx.90970.1.S1_s_at | 0.5 | CF806695 | Unclassified - Hypothetical Protein | | | |
| | | | | Supported by cDNA | | | |
| 10 | GmaAffx.23289.1.S1_at | 0.499 | CD392087 | No Homology to Known Proteins | | | |
| 11 | Gma.8782.1.A1_at | 0.499 | AW309953 | Disease & Defense | | | Apoptotic ATPase |
| 12 | GmaAffx.72346.1.S1_at | 0.499 | BF424359 | Unclassified - Hypothetical Protein | | | Speckle-type POZ protein SPOP and |
| | | | | NOT Supported by cDNA | | | related proteins with TRAF, MATH and |
| | | | | | | | BTB/POZ domains |
| 13 | GmaAffx.77527.1.S1_at | 0.499 | BF423510 | Unclassified - Hypothetical Protein | | | GTPase Rab18, small G protein |
| | | | | Supported by cDNA | | | superfamily |
| 14 | GmaAffx.87922.1.S1_at | 0.499 | BU084547 | Energy | | | Pyrophosphate-dependent |
| | | | | | | | phosphofructo-1-kinase |
| 15 | GmaAffx.65393.1.S1_s_at | 0.499 | BU760770 | No Homology to Known Proteins | | | |
| 16 | GmaAffx.75708.1.S1_at | 0.497 | BF599014 | Unclassified - Hypothetical Protein | | | |
| | | | | Supported by cDNA | | | |
| 17 | Gma.6025.1.S1_at | 0.497 | CD399734 | Unclassified - Hypothetical Protein | | | |
| | | | | NOT Supported by cDNA | | | |
| 18 | GmaAffx.36972.2.S1_at | 0.496 | BI699120 | Unclassified - Hypothetical Protein | | | |
| | | | | NOT Supported by cDNA | | | |
| 19 | Gma.6528.2.S1_at | 0.496 | CA784209 | Transporter | | | Calcium transporting ATPase |
| 20 | Gma.7835.1.S1_at | 0.496 | CD410897 | Cell Growth & Division | | | |
| 21 | GmaAffx.6565.1.A1_at | 0.496 | CD402930 | No Homology to Known Proteins | | | |
| 22 | GmaAffx.71778.1.S1_at | 0.495 | BE346999 | Unclassified - Hypothetical Protein | | | |
| | | | | NOT Supported by cDNA | | | |
| 23 | GmaAffx.1128.1.S1_at | 0.495 | AW348608 | Unclassified - Protein with | | | Copper chaperone |
| | | | | Unknown Function | | | |
| 24 | GmaAffx.53585.2.S1_at | 0.495 | BE556399 | Protein Destination & Storage | | | Molecular chaperone (DnaJ superfamily) |
| 25 | GmaAffx.37484.2.S1_at | 0.494 | BQ629925 | Unclassified - Protein with | | | Predicted membrane protein, contains |
| | | | | Unknown Function | | | DoH and Cytochrome b-561/ferric |
| | | | | | | | reductase transmembrane domains |
| 26 | Gma.14155.3.S1_at | 0.494 | AI437500 | Unclassified - Hypothetical Protein | | | Clathrin assembly protein AP180 and |
| | | | | Supported by cDNA | | | related proteins, contain ENTH domain |
| 27 | Gma.1677.2.A1_at | 0.494 | AW348386 | Secondary Metabolism | | | |
| 28 | GmaAffx.50632.1.A1_at | 0.494 | BI969115 | Metabolism | | | Malate dehydrogenase |
| 29 | Gma.6722.1.S1_at | 0.494 | BE822185 | Unclassified - Hypothetical Protein | | | UDP-glucuronosyl and UDP-glucosyl |
| | | | | NOT Supported by cDNA | | | transferase |
| 30 | GmaAffx.62195.1.S1_at | 0.493 | AW472439 | No Homology to Known Proteins | | | |
| 31 | GmaAffx.11383.1.S1_at | 0.493 | BE822014 | Unclassified - Hypothetical Protein | | | Kinesin light chain |
| | | | | NOT Supported by cDNA | | | |
| 32 | Gma.9717.1.S1_at | 0.492 | BG653084 | Unclassified - Hypothetical Protein | | | Amino acid transporters |
| | | | | NOT Supported by cDNA | | | |
| 33 | GmaAffx.21553.1.S1_at | 0.492 | BE023327 | Protein Destination & Storage | | | Gelatinase A and related matrix |
| | | | | | | | metalloproteases |
| 34 | GmaAffx.90952.1.S1_at | 0.491 | CF806677 | Signal Transduction | Unclassified | | |
| 35 | Gma.3175.1.S1_at | 0.491 | AI736154 | Unclassified - Hypothetical Protein | | | |
| | | | | NOT Supported by cDNA | | | |
| 36 | GmaAffx.84871.1.S1_at | 0.491 | BF599103 | Signal Transduction | Protein Kinase | | |
| 37 | GmaAffx.56020.1.S1_at | 0.49 | BU080527 | Unclassified - Hypothetical Protein | | | |
| | | | | NOT Supported by cDNA | | | |
| 38 | GmaAffx.765.2.S1_at | 0.49 | AW509310 | Transporter | | | Amino acid transporters |
| 39 | GmaAffx.86283.1.S1_at | 0.49 | BI892496 | Unclassified - Hypothetical Protein | | | Predicted pyroglutamyl peptidase |
| | | | | Supported by cDNA | | | |
| 40 | Gma.9436.1.S1_at | 0.49 | CA802734 | Unclassified - Hypothetical Protein | | | |
| | | | | NOT Supported by cDNA | | | |
| 41 | Gma.15155.2.S1_a_at | 0.489 | BG789619 | Unclassified - Hypothetical Protein | | | |
| | | | | Supported by cDNA | | | |
| 42 | Gma.16441.1.S1_a_at | 0.489 | BU548048 | Unclassified - Hypothetical Protein | | | HGG motif-containing thioesterase |
| | | | | NOT Supported by cDNA | | | |
| 43 | Gma.15729.1.A1_at | 0.489 | BI971055 | Cell Growth & Division | | | |
| 44 | GmaAffx.11840.1.S1_at | 0.488 | AW349211 | Post-Transcription | | | Predicted E3 ubiquitin ligase |
| 45 | GmaAffx.60958.3.S1_at | 0.487 | BG881523 | No Homology to Known Proteins | | | |
| 46 | GmaAffx.77269.1.S1_at | 0.487 | BM093698 | No Homology to Known Proteins | | | |
| 47 | Gma.7120.1.S1_at | 0.486 | BM093481 | Metabolism | | | Alanine-glyoxylate aminotransferase |
| | | | | | | | AGT2 |
| 48 | GmaAffx.6818.1.A1_at | 0.486 | BU546572 | Metabolism | | | Iron/ascorbate family oxidoreductases |
| 49 | GmaAffx.80638.1.S1_at | 0.486 | AW396070 | Unclassified - Hypothetical Protein | | | |
| | | | | NOT Supported by cDNA | | | |
| 50 | GmaAffx.77190.1.S1_at | 0.485 | BQ741890 | Metabolism | | | |
| 51 | GmaAffx.67456.1.S1_at | 0.485 | BM893057 | No Homology to Known Proteins | | | |
| 52 | GmaAffx.89049.1.S1_s_at | 0.485 | CK606168 | Intracellular Traffic | | | Annexin |
| 53 | GmaAffx.11419.1.S1_at | 0.485 | AI900831 | No Homology to Known Proteins | | | |
| 54 | Gma.197.1.S1_at | 0.484 | U55874.1 | Metabolism | Amino Acid | | Asparagine synthase (glutamine- |
| | | | | | Biosynthesis/Metabolism | | hydrolyzing) |
| 55 | GmaAffx.87550.1.S1_at | 0.484 | BM954718 | Disease & Defense | | | |
| 56 | GmaAffx.78790.1.S1_at | 0.483 | BQ298762 | Transporter | | | P-type ATPase |
| 57 | Gma.10857.2.S1_s_at | 0.483 | BQ612778 | Unclassified - Hypothetical Protein | | | Fasciclin and related adhesion |
| | | | | Supported by cDNA | | | glycoproteins |
| 58 | GmaAffx.87721.1.S1_at | 0.483 | AW156448 | Intracellular Traffic | | | Rho GDP-dissociation inhibitor |
| 59 | Gma.3400.1.S1_at | 0.482 | BQ627988 | No Homology to Known Proteins | | | |
| 60 | Gma.2771.1.S1_at | 0.481 | AW309763 | Unclassified - Hypothetical Protein | | | |
| | | | | Supported by cDNA | | | |
| 61 | Gma.10701.2.S1_at | 0.481 | AW101512 | No Homology to Known Proteins | | | |
| 62 | GmaAffx.53585.1.S1_at | 0.481 | BI471867 | Protein Destination & Storage | | | Molecular chaperone (DnaJ superfamily) |
| 63 | GmaAffx.77622.1.S1_at | 0.48 | BI674271 | Transcription | Transcription | Zinc Finger | |
| | | | | | Factor | | |
| 64 | GmaAffx.74868.1.S1_at | 0.48 | CA820573 | No Homology to Known Proteins | | | |
| 65 | GmaAffx.2412.1.S1_at | 0.48 | AI961002 | Secondary Metabolism | GA Biosynthesis | | Iron/ascorbate family oxidoreductases |
| 66 | GmaAffx.88251.1.S1_s_at | 0.48 | BU964415 | Disease & Defense | | | Soluble epoxide hydrolase |
| 67 | Gma.17798.1.S1_at | 0.479 | BU084728 | Unclassified - Hypothetical Protein | | | |
| | | | | Supported by cDNA | | | |
| 68 | Gma.11334.1.S1_a_at | 0.479 | BI967327 | Cell Structure | | | O-methyltransferase |
| 69 | GmaAffx.49130.1.S1_at | 0.479 | BE555035 | No Homology to Known Proteins | | | |
| 70 | GmaAffx.37484.1.S1_at | 0.479 | BG651852 | Unclassified - Protein with | | | Predicted membrane protein, contains |
| | | | | Unknown Function | | | DoH and Cytochrome b-561/ferric |
| | | | | | | | reductase transmembrane domains |
| 71 | Gma.11341.3.S1_s_at | 0.479 | BM177332 | Unclassified - Hypothetical Protein | | | |
| | | | | NOT Supported by cDNA | | | |
| 72 | GmaAffx.47590.1.S1_at | 0.478 | CA853861 | Metabolism | | | UDP-glucuronosyl and UDP-glucosyl |
| | | | | | | | transferase |
| 73 | GmaAffx.499.1.S1_at | 0.478 | BE058375 | Transcription | Transcription | MYB | Transcription factor, Myb superfamily |
| | | | | | Factor | | |
| 74 | GmaAffx.90097.1.S1_at | 0.477 | CF805822 | Cell Structure | | | |
| 75 | Gma.15499.1.S1_at | 0.477 | AW348149 | Energy | | | |
| 76 | GmaAffx.83232.1.S1_at | 0.477 | BE023128 | Disease & Defense | | | |
| 77 | Gma.6075.1.S1_a_at | 0.476 | AW317183 | Disease & Defense | | | |
| 78 | Gma.3160.1.S1_at | 0.476 | BG507816 | Energy | | | Thioredoxin |
| 79 | Gma.8525.1.S1_s_at | 0.476 | AI966178 | Disease & Defense | | | |
| 80 | Gma.18052.1.S1_at | 0.476 | BF595464 | Transporter | | | |
| 81 | Gma.385.1.S1_at | 0.475 | BE347563 | Intracellular Traffic | | | Annexin |
| 82 | Gma.11135.1.S1_at | 0.475 | AI856724 | Energy | | | ATP-citrate lyase |
| 83 | Gma.1445.1.S1_a_at | 0.475 | BQ454241 | Metabolism | | | Flavonol reductase/cinnamoyl-CoA |
| | | | | | | | reductase |
| 84 | GmaAffx.35729.2.S1_at | 0.474 | BM732483 | Unclassified - Hypothetical Protein | | | |
| | | | | Supported by cDNA | | | |
| 85 | Gma.13257.1.A1_at | 0.474 | CD396346 | Unclassified - Protein with | | | |
| | | | | Unknown Function | | | |
| 86 | Gma.9228.1.S1_at | 0.473 | CD417435 | Unclassified - Hypothetical Protein | | | |
| | | | | NOT Supported by cDNA | | | |
| 87 | Gma.1749.3.S1_at | 0.472 | BI784667 | Protein Destination & Storage | Ubiquitin | | FOG: Predicted E3 ubiquitin ligase |
| | | | | | Degradation | | |
| 88 | Gma.14563.1.S1_s_at | 0.471 | CD399224 | Energy | | | |
| 89 | Gma.16288.1.S1_at | 0.471 | AW598590 | Unclassified - Hypothetical Protein | | | Fasciclin and related adhesion |
| | | | | NOT Supported by cDNA | | | glycoproteins |
| 90 | GmaAffx.85342.1.S1_at | 0.471 | BI426916 | Unclassified - Hypothetical Protein | | | |
| | | | | NOT Supported by cDNA | | | |
| 91 | GmaAffx.15471.3.S1_s_at | 0.47 | BE346777 | Transcription Factor | Transcription | MYB | Transcription factor, Myb superfamily |
| 92 | Gma.17861.1.S1_at | 0.47 | AW310110 | Cell Structure | | | |
| 93 | Gma.1294.1.S1_at | 0.47 | CD395653 | Unclassified - Hypothetical Protein | | | |
| | | | | Supported by cDNA | | | |
| 94 | GmaAffx.36017.1.S1_at | 0.469 | BG154593 | Unclassified - Hypothetical Protein | | | |
| | | | | Supported by cDNA | | | |
| 95 | Gma.2470.1.S1_s_at | 0.468 | AI442629 | Cell Structure | | | |
| 96 | GmaAffx.28690.1.S1_at | 0.467 | CA799411 | Transcription | Transcription | Homeodomain | |
| | | | | | Factor | | |
| 97 | Gma.17763.1.A1_at | 0.467 | BI321757 | Unclassified - Hypothetical Protein | | | Diadenosine and diphosphoinositol |
| | | | | Supported by cDNA | | | polyphosphate phosphohydrolase |
| 98 | Gma.8143.1.S1_at | 0.466 | BG725425 | Signal Transduction | MAPK | | Mitogen-activated protein kinase |
| 99 | GmaAffx.68099.1.S1_at | 0.466 | AW734055 | No Homology to Known Proteins | | | |
| 100 | Gma.3881.1.S1_at | 0.465 | X92437.1 | Metabolism | Cytochrome | CYP73A | Cytochrome P450 CYP2 subfamily |
| | | | | | P450 | | |
| 101 | Gma.1701.1.S1_at | 0.464 | M67449.1 | Signal Transduction | MAPKKK | Raf | Tyrosine kinase specific for activated |
| | | | | | | | (GTP-bound) p21cdc42Hs |
| 102 | GmaAffx.3548.1.S1_at | 0.464 | CA851347 | Transporter | | | Apolipoprotein D/Lipocalin |
| 103 | Gma.12584.2.A1_at | 0.464 | CD406878 | Metabolism | | | Long-chain acyl-CoA synthetases (AMP- |
| | | | | | | | forming) |
| 104 | GmaAffx.8113.1.S1_at | 0.463 | BE659323 | Disease & Defense | | | |
| 105 | GmaAffx.64885.1.S1_at | 0.462 | BQ081653 | No Homology to Known Proteins | | | |
| 106 | Gma.1445.3.S1_a_at | 0.461 | BE805686 | Metabolism | | | Flavonol reductase/cinnamoyl-CoA |
| | | | | | | | reductase |
| 107 | Gma.8072.1.S1_a_at | 0.461 | BQ627840 | Transcription | Transcription | Basic Leucine | |
| | | | | | Factor | Zipper (bZIP) | |
| 108 | GmaAffx.68588.1.S1_at | 0.46 | BI787152 | Unclassified - Hypothetical Protein | | | |
| | | | | Supported by cDNA | | | |
| 109 | GmaAffx.21943.1.S1_at | 0.46 | AW101735 | Unclassified - Hypothetical Protein | | | |
| | | | | NOT Supported by cDNA | | | |
| 110 | GmaAffx.24382.1.S1_at | 0.46 | BM886764 | No Homology to Known Proteins | | | |
| 111 | GmaAffx.62941.1.S1_at | 0.459 | AW597498 | Unclassified - Protein with | | | |
| | | | | Unknown Function | | | |
| 112 | GmaAffx.80474.1.S1_at | 0.458 | AW707170 | Signal Transduction | MAPKKK | MEKK | MEKK and related serine/threonine |
| | | | | | | | protein kinases |
| 113 | GmaAffx.84721.2.S1_s_at | 0.458 | BG041982 | Metabolism | | | Hydroxymethylglutaryl-CoA lyase |
| 114 | GmaAffx.69076.1.S1_at | 0.458 | BM525610 | Unclassified - Hypothetical Protein | | | |
| | | | | NOT Supported by cDNA | | | |
| 115 | Gma.13458.1.A1_at | 0.458 | CD398733 | Protein Destination & Storage | Ubiquitin | | FOG: Predicted E3 ubiquitin ligase |
| | | | | | Degradation | | |
| 116 | Gma.16896.1.A1_at | 0.457 | CD416188 | No Homology to Known Proteins | | | |
| 117 | GmaAffx.15204.1.A1_at | 0.457 | BU549835 | No Homology to Known Proteins | | | |
| 118 | Gma.412.1.S1_at | 0.456 | BE607835 | Protein Destination & Storage | | | Ubiquitin-protein ligase |
| 119 | Gma.2253.1.S1_at | 0.456 | AF169018.1 | Metabolism | | | Reductases with broad range of substrate |
| | | | | | | | specificities |
| 120 | GmaAffx.51044.1.S1_at | 0.456 | BM522670 | Unclassified - Hypothetical Protein | | | Copine |
| | | | | Supported by cDNA | | | |
| 121 | GmaAffx.85565.1.S1_at | 0.456 | BE611082 | Signal Transduction | Calcium Binding | Calcium | Ca2+/calmodulin-dependent protein |
| | | | | | | Dependent | kinase, EF-Hand protein superfamily |
| | | | | | | Protein Kinase | |
| 122 | Psojae_rRNA_482_at | 0.455 | | | | | |
| 123 | Gma.14611.1.S1_at | 0.454 | CA852673 | Unclassified - Protein with | | | |
| | | | | Unknown Function | | | |
| 124 | GmaAffx.50617.1.S1_at | 0.454 | AW101420 | Unclassified - Hypothetical Protein | | | |
| | | | | Supported by cDNA | | | |
| 125 | Gma.4374.1.S1_at | 0.454 | BM178538 | Metabolism | Cytochrome | CYP76C | Cytochrome P450 CYP2 subfamily |
| | | | | | P450 | | |
| 126 | GmaAffx.93017.1.S1_at | 0.454 | CF808742 | Disease & Defense | | | Soluble epoxide hydrolase |
| 127 | Gma.4673.1.A1_at | 0.453 | BG508468 | No Homology to Known Proteins | | | |
| 128 | GmaAffx.21773.1.S1_at | 0.453 | BU763882 | Unclassified - Hypothetical Protein | | | |
| | | | | NOT Supported by cDNA | | | |
| 129 | GmaAffx.24521.1.S1_at | 0.453 | BU761767 | Unclassified - Hypothetical Protein | | | |
| | | | | NOT Supported by cDNA | | | |
| 130 | Gma.15563.1.S1_at | 0.453 | CD415760 | Unclassified - Hypothetical Protein | | | |
| | | | | Supported by cDNA | | | |
| 131 | GmaAffx.10297.1.S1_at | 0.453 | BE475510 | Metabolism | | | Beta-galactosidase |
| 132 | Gma.9842.1.S1_at | 0.452 | BM528240 | Unclassified - Hypothetical Protein | | | Leucine rich repeat proteins, some |
| | | | | Supported by cDNA | | | proteins contain F-box |
| 133 | GmaAffx.90853.1.S1_at | 0.452 | CF806578 | No Homology to Known Proteins | | | |
| 134 | GmaAffx.54778.1.S1_at | 0.451 | BI322039 | No Homology to Known Proteins | | | |
| 135 | Gma.13042.1.S1_at | 0.45 | CD390874 | Unclassified - Hypothetical Protein | | | |
| | | | | NOT Supported by cDNA | | | |
| 136 | Gma.17136.1.S1_s_at | 0.449 | BE022719 | Energy | | | Cytochrome b |
| 137 | GmaAffx.92463.1.S1_at | 0.448 | CF808188 | Metabolism | | | NADP+-dependent malic enzyme |
| 138 | GmaAffx.20688.1.S1_at | 0.448 | BG157357 | Unclassified - Protein with | | | Fatty acyl-CoA |
| | | | | Unknown Function | | | elongase/Polyunsaturated fatty acid |
| | | | | | | | specific elongation enzyme |
| 139 | GmaAffx.81388.1.S1_at | 0.448 | BF424571 | Signal Transduction | Protein Kinase | | Serine/threonine protein kinase |
| 140 | GmaAffx.11892.1.S1_at | 0.447 | BE820605 | Secondary Metabolism | | | Iron/ascorbate family oxidoreductases |
| 141 | GmaAffx.91688.1.S1_at | 0.446 | CF807413 | No Homology to Known Proteins | | | |
| 142 | GmaAffx.35278.1.S1_at | 0.445 | BE346991 | Disease & Defense | | | |
| 143 | GmaAffx.88786.1.A1_s_at | 0.444 | CK606552 | Cell Structure | | | O-methyltransferase |
| 144 | GmaAffx.73088.1.S1_at | 0.444 | BE475110 | Unclassified - Hypothetical Protein | | | |
| | | | | Supported by cDNA | | | |
| 145 | Gma.4305.3.S1_a_at | 0.444 | BG044173 | Transporter | Ion Channel | Cyclic | K+-channel ERG and related proteins, |
| | | | | | Families | nucleotide | contain PAS/PAC sensor domain |
| | | | | | | gated channel | |
| | | | | | | family | |
| 146 | GmaAffx.77097.1.S1_at | 0.443 | BG237643 | No Homology to Known Proteins | | | |
| 147 | GmaAffx.18351.1.S1_at | 0.443 | BI472051 | Unclassified - Hypothetical Protein | | | FOG: Armadillo/beta-catenin-like |
| | | | | Supported by cDNA | | | repeats |
| 148 | Gma.4437.1.S1_s_at | 0.443 | BQ628831 | Secondary Metabolism | Flavonoid | | Flavonol reductase/cinnamoyl-CoA |
| | | | | | Biosynthesis | | reductase |
| 149 | GmaAffx.18599.1.S1_at | 0.443 | BI787464 | Secondary Metabolism | Amines | | Amine oxidase |
| | | | | | Biosynthesis | | |
| 150 | Gma.5418.2.S1_at | 0.442 | BI469627 | Cell Growth & Division | | | |
| 151 | Gma.5218.1.A1_at | 0.442 | CD392232 | Metabolism | | | Predicted carbonic anhydrase involved |
| | | | | | | | in protection against oxidative damage |
| 152 | GmaAffx.84795.1.S1_at | 0.442 | BG046191 | Unclassified - Hypothetical Protein | | | |
| | | | | NOT Supported by cDNA | | | |
| 153 | Gma.415.1.A1_at | 0.442 | AW350315 | Protein Destination & Storage | | | FOG: Predicted E3 ubiquitin ligase |
| 154 | Gma.1123.1.S1_at | 0.441 | CD402207 | Unclassified - Hypothetical Protein | | | |
| | | | | Supported by cDNA | | | |
| 155 | GmaAffx.44484.1.S1_at | 0.441 | BE475213 | Unclassified - Hypothetical Protein | | | |
| | | | | NOT Supported by cDNA | | | |
| 156 | GmaAffx.93430.1.S1_s_at | 0.439 | CF809155 | Disease & Defense | | | Purple (tartrate-resistant) acid |
| | | | | | | | phosphatase |
| 157 | GmaAffx.34775.1.S1_at | 0.439 | AI794730 | Unclassified - Protein with | | | FOG: Predicted E3 ubiquitin ligase |
| | | | | Unknown Function | | | |
| 158 | GmaAffx.17961.2.S1_at | 0.438 | BG511562 | Protein Destination & Storage | | | Molecular chaperones HSP70/HSC70, |
| | | | | | | | HSP70 superfamily |
| 159 | GmaAffx.9482.2.S1_at | 0.438 | AI900615 | Unclassified - Hypothetical Protein | | | Predicted guanosine polyphosphate |
| | | | | NOT Supported by cDNA | | | pyrophosphohydrolase/synthase |
| 160 | Gma.3589.1.S1_a_at | 0.437 | AW101438 | Secondary Metabolism | GA Biosynthesis | | Iron/ascorbate family oxidoreductases |
| 161 | Gma.6245.1.S1_at | 0.437 | CA938361 | No Homology to Known Proteins | | | |
| 162 | Gma.2486.1.S1_at | 0.437 | BF219523 | Unclassified - Hypothetical Protein | | | Iron/ascorbate family oxidoreductases |
| | | | | NOT Supported by cDNA | | | |
| 163 | Gma.12494.1.S1_at | 0.437 | BG839922 | Protein Destination & Storage | Ubiquitin | E3 Ubiquitin | FOG: Predicted E3 ubiquitin ligase |
| | | | | | Degradation | Ligase | |
| 164 | GmaAffx.51468.1.S1_at | 0.437 | BQ628796 | No Homology to Known Proteins | | | |
| 165 | Gma.17062.1.S1_at | 0.436 | AW277440 | No Homology to Known Proteins | | | |
| 166 | Gma.892.1.A1_at | 0.435 | BE822836 | Unclassified - Protein with | | | Prolyl 4-hydroxylase alpha subunit |
| | | | | Unknown Function | | | |
| 167 | GmaAffx.74304.1.S1_at | 0.435 | BU577360 | Unclassified - Hypothetical Protein | | | |
| | | | | NOT Supported by cDNA | | | |
| 168 | GmaAtTx.81253.1.S1 _at | 0.434 | BE807371 | Unclassified - Protein with | | | |
| | | | | Unknown Function | | | |
| 169 | Gma.18084.1.S1_at | 0.434 | BI317557 | Disease & Defense | | | Ferric reductase, NADH/NADPH |
| | | | | | | | oxidase and related proteins |
| 170 | GmaAffx.91108.1.S1_at | 0.434 | CF806833 | Unclassified - Hypothetical Protein | | | |
| | | | | Supported by cDNA | | | |
| 171 | Gma.1190.1.S1_at | 0.434 | AF279267.1 | Disease & Defense | | | Acyl-CoA synthetase |
| 172 | GmaAffx.92192.1.S1_s_at | 0.434 | CF806406 | Disease & Defense | | | |
| 173 | Gma.10281.1.S1_at | 0.433 | CA852560 | Unclassified - Protein with | | | Predicted E3 ubiquitin ligase |
| | | | | Unknown Function | | | |
| 174 | GmaAffx.91814.1.S1_at | 0.432 | CF807539 | Unclassified - Hypothetical Protein | | | |
| | | | | Supported by cDNA | | | |
| 175 | GmaAffx.84751.1.A1_at | 0.432 | AW349702 | Transcription | Transcription | MYB | Transcription factor, Myb superfamily |
| | | | | | Factor | | |
| 176 | Gma.15773.1.A1_at | 0.431 | CD406280 | Metabolism | | | |
| 177 | Gma.12873.1.S1_at | 0.431 | CD393774 | No Homology to Known Proteins | | | |
| 178 | GmaAffx.9926.2.S1_at | 0.431 | BF597587 | Unclassified - Hypothetical Protein | | | |
| | | | | Supported by cDNA | | | |
| 179 | Gma.14312.1.S1_at | 0.431 | CA852635 | No Homology to Known Proteins | | | |
| 180 | Gma.15470.1.S1_at | 0.43 | BU546303 | Unclassified - Hypothetical Protein | | | |
| | | | | Supported by cDNA | | | |
| 181 | GmaAffx.15222.1.S1_at | 0.43 | BU550700 | Unclassified - Hypothetical Protein | | | |
| | | | | NOT Supported by cDNA | | | |
| 182 | GmaAffx.4505.1.S1_at | 0.43 | AW306682 | Unclassified - Protein with | | | Predicted NUD1X hydrolase FGF-2 and |
| | | | | Unknown Function | | | related proteins |
| 182 | GmaAffx.4505.1.S1_at | 0.43 | AW306682 | Unclassified - Protein with | | | Cdc4 and related F-box and WD-40 |
| | | | | Unknown Function | | | proteins |
| 183 | GmaAffx.90827.1.S1_at | 0.43 | CF806552 | Unclassified - Hypothetical Protein | | | Molecular chaperone (small heat-shock |
| | | | | Supported by cDNA | | | protein Hsp26/Hsp42) |
| 184 | GmaAffx.92184.1.S1_s_at | 0.43 | CF807909 | Unclassified - Protein with | | | |
| | | | | Unknown Function | | | |
| 185 | GmaAffx.89971.1.S1_s_at | 0.429 | CF805696 | Intracellular Traffic | | | Annexin |
| 186 | GmaAffx.66982.1.S1_at | 0.428 | BI970847 | Protein Destination & Storage | | | Molecular chaperone (small heat-shock |
| | | | | | | | protein Hsp26/Hsp42) |
| 187 | GmaAffx.45236.1.S1_at | 0.428 | BG156638 | Disease & Defense | | | Purple (tartrate-resistant) acid |
| | | | | | | | phosphatase |
| 188 | Gma.6075.1.S1_at | 0.428 | AW317183 | Disease & Defense | | | |
| 189 | Gma.10795.1.S1_at | 0.428 | AI442845 | Cell Structure | | | |
| 190 | GmaAffx.55244.1.A1_at | 0.427 | BU549116 | No Homology to Known Proteins | | | |
| 191 | GmaAffx.90104.1.S1_s_at | 0.427 | CF805829 | Transporter | | | Permease of the major facilitator |
| | | | | | | | superfamily |
| 192 | Gma.3969.2.S1_at | 0.426 | CD412522 | Energy | | | |
| 193 | Gma.16547.1.S1_at | 0.426 | BU545050 | Transcription | Transcription | WRKY | |
| | | | | | Factor | | |
| 194 | GmaAffx.74211.1.S1_at | 0.426 | BE820853 | Protein Destination & Storage | | | Aspartyl protease |
| 195 | GmaAffx.42667.3.S1_at | 0.426 | AW306785 | Transcription | Transcription | GRAS | |
| | | | | | Factor | | |
| 196 | Gma.2772.1.S1_at | 0.426 | BG238134 | No Homology to Known Proteins | | | |
| 197 | GmaAffx.18742.1.S1_at | 0.425 | BI970450 | Unclassified - Hypothetical Protein | | | Predicted membrane protein |
| | | | | Supported by cDNA | | | |
| 198 | Gma.1947.1.S1_at | 0.425 | BI968172 | Transcription | Transcription | Homeodomain | Transcription factor MEIS1 and related |
| | | | | | Factor | | HOX domain proteins |
| 199 | Gma.5400.1.S1_at | 0.425 | BM093775 | No Homology to Known Proteins | | | |
| 200 | Gma.6709.1.S1_at | 0.424 | BE823291 | Signal Transduction | Protein Kinase | AtCIPKs | Serine/threonine protein kinase |
| 201 | Gma.11131.1.S1_at | 0.424 | BI968388 | Protein Destination & Storage | | | Molecular chaperone (small heat-shock |
| | | | | | | | protein Hsp26/Hsp42) |
| 202 | GmaAffx.72580.1.S1_at | 0.424 | BG157182 | Unclassified - Hypothetical Protein | | | |
| | | | | Supported by cDNA | | | |
| 203 | Gma.4432.1.S1_a_at | 0.423 | BQ610841 | Unclassified - Protein with | Hormone | | Voltage-gated shaker-like K+ channel, |
| | | | | Unknown Function | Induced | | subunit heta/KCNAB |
| 204 | GmaAffx.74194.1.S1_at | 0.423 | BI968058 | Metabolism | | | 17 beta-hydroxysteroid dehydrogenase |
| | | | | | | | type 3, HSD17B3 |
| 205 | GmaAffx.62375.1.S1_at | 0.423 | BQ612727 | Unclassified - Hypothetical Protein | | | |
| | | | | Supported by cDNA | | | |
| 206 | GmaAffx.65580.1.S1_at | 0.422 | CA799171 | Metabolism | | | |
| 207 | GmaAffx.81618.1.S1_at | 0.421 | AW706403 | No Homology to Known Proteins | | | |
| 208 | GmaAffx.16201.1.S1_at | 0.421 | BU550725 | No Homology to Known Proteins | | | |
| 209 | GmaAffx.55124.1.S1_at | 0.421 | BI970821 | Unclassified - Hypothetical Protein | | | Metacaspase involved in regulation of |
| | | | | Supported by cDNA | | | apoptosis |
| 210 | Gma.4755.1.S1_at | 0.421 | AW310232 | Unclassified - Hypothetical Protein | | | |
| | | | | NOT Supported by cDNA | | | |
| 211 | GmaAffx.9059.1.S1_at | 0.421 | AW423327 | No Homology to Known Proteins | | | |
| 212 | GmaAffx.51614.1.S1_at | 0.42 | BU089837 | No Homology to Known Proteins | | | |
| 213 | Gma.153.1.S1_x_at | 0.42 | Y10491.1 | Metabolism | Cytochrome | CYP82C | Cytochrome P450 CYP2 subfamily |
| | | | | | P450 | | |
| 214 | Gma.4680.1.A1_s_at | 0.418 | AW309707 | Unclassified - Protein with | | | |
| | | | | Unknown Function | | | |
| 215 | Gma.17288.1.A1_at | 0.418 | AW310692 | No Homology to Known Proteins | | | |
| 216 | Gma.5258.1.S1_at | 0.418 | BE658899 | No Homology to Known Proteins | | | |
| 217 | GmaAffx.2710.1.S1_at | 0.417 | BE822538 | Unclassified - Hypothetical Protein | | | Endoplasmic reticulum protein EP58. |
| | | | | Supported by cDNA | | | contains filamin rod domain and KDEL |
| | | | | | | | motif |
| 218 | GmaAffx.89049.1.S1_at | 0.417 | CK606168 | Intracellular Traffic | | | Annexin |
| 219 | Gma.6046.1.S1_at | 0.417 | BE022758 | Unclassified - Hypothetical Protein | | | |
| | | | | NOT Supported by cDNA | | | |
| 220 | Gma.4839.1.A1_at | 0.417 | AW310019 | Metabolism | | | Branched chain aminotransferase |
| | | | | | | | BCAT1, pyridoxal phosphate enzymes |
| | | | | | | | type IV superfamily |
| 221 | GmaAffx.26320.1.S1_at | 0.416 | BE440416 | Unclassified - Hypothetical Protein Supported | | | |
| | | | | by cDNA | | | |
| 222 | Gma.10969.1.S1_x_at | 0.416 | U36191.1 | Metabolism | | | |
| 223 | GmaAffx.90234.1.S1_at | 0.416 | CF805959 | Transcription | Transcription | Zinc Finger | FOG: Zn-finger |
| | | | | | Factor | | |
| 224 | GmaAffx.28578.1.S1_at | 0.416 | BQ094224 | Disease & Defense | | | |
| 225 | GmaAffx.59762.1.S1_at | 0.416 | BI971811 | Unclassified - Protein with | | | |
| | | | | Unknown Function | | | |
| 226 | Gma.18020.1.S1_at | 0.415 | AW309711 | Signal Transduction | Protein Kinase | | Serine/threonine protein kinase |
| 227 | GmaAffx.10580.1.S1_at | 0.414 | BF424010 | Transporter | | | Amino acid transporters |
| 228 | GmaAffx.12369.1.A1_at | 0.414 | CD412820 | Unclassified - Hypothetical Protein | | | |
| | | | | Supported by cDNA | | | |
| 229 | Gma.5256.2.S1_at | 0.414 | AI441316 | Unclassified - Hypothetical Protein | | | |
| | | | | NOT Supported by cDNA | | | |
| 230 | GmaAffx.1890.1.S1_s_at | 0.414 | BI970941 | Transcription | Transcription Factor | Heat Shock | Heat shock transcription factor |
| 231 | Gma.10266.1.S1_at | 0.414 | BG157207 | No Homology to Known Proteins | | | |
| 232 | Gma.17535.1.S1_at | 0.413 | BM178659 | Unclassified - Hypothetical Protein | | | Uncharacterized conserved protein, |
| | | | | Supported by cDNA | | | contains double-stranded beta-helix |
| | | | | | | | domain |
| 233 | Gma.7836.2.S1_at | 0.413 | BI425718 | Unclassified - Hypothetical Protein | | | |
| | | | | NOT Supported by cDNA | | | |
| 234 | Gma.17502.2.S1_at | 0.413 | BG046201 | Transcription | Transcription | Unclassified | Zuotin and related molecular chaperones |
| | | | | | Factor | | (DnaJ superfamily), contains DNA- |
| | | | | | | | binding domains |
| 235 | Gma.4839.2.S1_at | 0.412 | BI974385 | Metabolism | | | Branched chain aminotransferase |
| | | | | | | | BCAT1, pyridoxal phosphate enzymes |
| | | | | | | | type IV superfamily |
| 236 | Gma.11195.1.S1_at | 0.412 | CD417406 | Transporter | | | Apolipoprotein D/Lipocalin |
| 237 | Gma.11028.1.S1_at | 0.412 | BE823084 | Metabolism | | | Glyoxylate/hydroxypyruvate reductase |
| | | | | | | | (D-isomer-specific 2-hydroxy acid |
| | | | | | | | dehydrogenase superfamily) |
| 238 | GmaAffx.76346.1.A1_at | 0.412 | BQ612378 | Energy | | | 1,4-benzoquinone reductase-like; Trp |
| | | | | | | | repressor binding protein-like/protoplast- |
| | | | | | | | secreted protein |
| 239 | GmaAffx.91108.1.S1_s_at | 0.411 | CF806833 | Unclassified - Hypothetical Protein | | | |
| | | | | Supported by cDNA | | | |
| 240 | Gma.4455.2.S1_at | 0.409 | BE059560 | Signal Transduction | Protein Kinase | | Serine/threonine protein kinase |
| 241 | GmaAffx.26299.1.S1_at | 0.408 | BQ299016 | Signal Transduction | Protein Kinase | | |
| 242 | Gma.7120.2.S1_a_at | 0.408 | BG041285 | Metabolism | | | Alanine-glyoxylate aminotransferase |
| | | | | | | | AGT2 |
| 243 | Gma.14295.1.S1_at | 0.405 | CA852253 | No Homology to Known Proteins | | | |
| 244 | Gma.6761.1.A1_at | 0.405 | BQ453407 | Cell Structure | | | Beta-glucosidase, lactase |
| | | | | | | | phlorizinhydrolase, and related proteins |
| 245 | GmaAffx.26859.1.S1_s_at | 0.405 | BM085620 | Disease & Defense | | | Glutathione S-transferase |
| 246 | GmaAffx.83564.1.S1_at | 0.405 | BU578309 | No Homology to Known Proteins | | | |
| 247 | Gma.17642.1.S1_at | 0.405 | BU550265 | Unclassified - Hypothetical Protein | | | BP1/LBP/CETP family protein |
| | | | | Supported by cDNA | | | |
| 248 | Gma.3283.1.S1_at | 0.404 | BQ298733 | No Homology to Known Proteins | | | |
| 249 | Gma.7487.2.A1_at | 0.403 | AW349599 | Transporter | | | Amino acid transporters |
| 250 | GmaAffx.7738.1.S1_s_at | 0.403 | BE058056 | No Homology to Known Proteins | | | |
| 251 | Gma.4124.1.S1_at | 0.403 | BI787263 | Transcription | Transcription | Homeodomain | |
| | | | | | Factor | | |
| 252 | GmaAffx.89141.1.S1_at | 0.403 | CK605765 | Cell Structure | | | O-methyltransferase |
| 253 | Gma.7051.1.A1_at | 0.403 | BU544101 | Disease & Defense | | | Metacaspase involved in regulation of |
| | | | | | | | apoptosis |
| 254 | Gma.10854.1.S1_at | 0.403 | CA802334 | No Homology to Known Proteins | | | |
| 255 | GmaAffx.81502.1.S1_at | 0.403 | CD404946 | Unclassified - Hypothetical Protein | | | Uncharacterized conserved protein |
| | | | | NOT Supported by cDNA | | | |
| 256 | GmaAffx.53108.1.S1_at | 0.403 | BM093748 | Signal Transduction | Protein Kinase | | Serine/threonine protein kinase |
| 257 | Gma.15469.1.S1_at | 0.402 | BU546655 | Cell Structure | | | Alpha tubulin |
| 258 | GmaAffx.24809.1.S1_at | 0.402 | CA820582 | Unclassified - Hypothetical Protein | | | |
| | | | | NOT Supported by cDNA | | | |
| 259 | Gma.1114.1.S1_s_at | 0.402 | CD403139 | Unclassified - Hypothetical Protein | | | |
| | | | | NOT Supported by cDNA | | | |
| 260 | GmaAffx.92230.1.S1_at | 0.402 | CF807302 | Disease & Defense | | | |
| 261 | GmaAffx.63822.1.S1_at | 0.402 | BM520838 | Disease & Defense | | | Purple (tartrate-resistant) acid |
| | | | | | | | phosphatase |
| 262 | GmaAffx.84607.2.S1_at | 0.401 | BU080867 | Unclassified - Hypothetical Protein | | | |
| | | | | Supported by cDNA | | | |
| 263 | GmaAffx.54085.1.S1_at | 0.401 | BI970762 | Unclassified - Hypothetical Protein | | | |
| | | | | Supported by cDNA | | | |
| 264 | GmaAffx.50347.1.S1_at | 0.4 | BQ740576 | Signal Transduction | Protein Kinase | | |
| 265 | GmaAffx.1694.1.S1_at | 0.4 | BQ454286 | Transcription | Transcription | MYB | |
| | | | | | Factor | | |
| 266 | GmaAffx.60479.4.S1_at | 0.4 | BG507369 | Transcription | Transcription | Homeodomain | Transcription factor MEIS1 and related |
| | | | | | Factor | | HOX domain proteins |
| 267 | GmaAffx.6824.2.S1_at | 0.399 | BQ094655 | Cell Structure | | | |
| 268 | Gma.3589.1.S1_at | 0.399 | AW101438 | Secondary Metabolism | GA Biosynthesis | | Iron/ascorbate family oxidoreductases |
| 269 | Gma.16585.1.S1_x_at | 0.399 | CA850597 | Metabolism | | | Predicted carbonic anhydrase involved |
| | | | | | | | in protection against oxidative damage |
| 270 | GmaAffx.57967.1.S1_at | 0.399 | AW102170 | Protein Destination & Storage | | | |
| 271 | Gma.10885.1.S1_at | 0.398 | BQ610245 | Unclassified - Hypothetical Protein | | | Predicted kinase |
| | | | | Supported by cDNA | | | |
| 272 | Gma.10626.3.S1_at | 0.397 | BE022458 | Secondary Metabolism | | | Iron/ascorbate family oxidoreductases |
| 273 | Gma.5038.1.A1_at | 0.397 | BE023631 | Disease & Defense | | | Acyl-CoA synthetase |
| 274 | GmaAffx.43786.2.S1_at | 0.396 | BU080930 | Signal Transduction | Histidine Kinase | | Sensory transduction histidine kinase |
| 275 | GmaAffx.24844.1.S1_at | 0.396 | BU765373 | Unclassified - Hypothetical Protein | | | Uncharacterized conserved protein |
| | | | | Supported by cDNA | | | |
| 276 | GmaAffx.27787.1.A1_at | 0.396 | BG789758 | Transporter | | | Transporter, ABC superfamily (Breast |
| | | | | | | | cancer resistance protein) |
| 277 | Gma.1380.1.S1_at | 0.396 | CA935076 | Metabolism | | | |
| 278 | Gma.4260.1.S1_at | 0.396 | BM177256 | Unclassified - Hypothetical Protein | | | |
| | | | | NOT Supported by cDNA | | | |
| 279 | Gma.5709.1.S1_at | 0.395 | BG157556 | Unclassified - Protein with | | | |
| | | | | Unknown Function | | | |
| 280 | GmaAffx.22297.2.S1_at | 0.395 | BM271210 | Transcription | Transcription | WRKY | |
| | | | | | Factor | | |
| 281 | Gma.5578.2.S1_a_at | 0.394 | AW310579 | Metabolism | Cytochrome | CYP98A | Cytochrome P450 CYP2 subfamily |
| | | | | | P450 | | |
| 282 | GmaAffx.28870.1.S1_at | 0.393 | BG726532 | Unclassified - Hypothetical Protein | | | |
| | | | | Supported by cDNA | | | |
| 283 | GmaAffx.73195.1.S1_at | 0.393 | BI701045 | Secondary Metabolism | Flavonoid | | Flavonol reductase/cinnamoyl-CoA |
| | | | | | Biosynthesis | | reductase |
| 284 | Gma.16147.1.S1_at | 0.393 | BE473577 | Transcription | Transcription | Basic Helix- | |
| | | | | | Factor | Loop-Helix | |
| | | | | | | (bHLH) | |
| 285 | GmaAffx.23789.1.S1_at | 0.392 | BU551014 | Unclassified - Hypothetical Protein | | | |
| | | | | Supported by cDNA | | | |
| 286 | GmaAffx.37636.1.S1_at | 0.392 | BQ610347 | Metabolism | Amino Acid | | Alanine-glyoxylate aminotransferase |
| | | | | | Biosynthesis/Metabolism | | AGT2 |
| 287 | Gma.7047.1.S1_s_at | 0.392 | CD417401 | Unclassified - Protein with | | | |
| | | | | Unknown Function | | | |
| 288 | GmaAffx.3726.1.A1_at | 0.391 | BE822942 | Unclassified - Hypothetical Protein | | | |
| | | | | Supported by cDNA | | | |
| 289 | Gma.5215.1.S1_at | 0.391 | AI988141 | Unclassified - Hypothetical Protein | | | BCL2-associated athanogene-like |
| | | | | Supported by cDNA | | | proteins and related BAG family |
| | | | | | | | chaperone regulators |
| 290 | Gma.3745.1.S1_at | 0.389 | AW156339 | Secondary Metabolism | Amines | | Amine oxidase |
| | | | | | Biosynthesis | | |
| 291 | Gma.16336.1.S1_at | 0.389 | BE659555 | Protein Destination & Storage | | | Predicted pilin-like transcription factor |
| 292 | Gma.16276.2.S1_a_at | 0.388 | BG316414 | Unclassified - Hypothetical Protein | | | FOG: PPR repeat |
| | | | | Supported by cDNA | | | |
| 293 | GmaAffx.68853.1.S1_at | 0.388 | CA803108 | Unclassified - Hypothetical Protein | | | |
| | | | | Supported by cDNA | | | |
| 294 | GmaAffx.73021.1.S1_at | 0.387 | BE556340 | Disease & Defense | | | |
| 295 | GmaAffx.80899.1.S1_at | 0.387 | AW348263 | Unclassified - Hypothetical Protein | | | |
| | | | | Supported by cDNA | | | |
| 296 | GmaAffx.77052.1.S1_at | 0.387 | BE556140 | Transporter | Inorganic Solute | Putative | |
| | | | | | Cotransporters | potassium | |
| | | | | | | transporter | |
| | | | | | | family | |
| 297 | GmaAffx.77052.2.A1_at | 0.387 | BG155285 | Transporter | Inorganic Solute | Putative | |
| | | | | | Cotransporters | potassium | |
| | | | | | | transporter | |
| | | | | | | family | |
| 298 | GmaAffx.17924.1.S1_at | 0.387 | BG157338 | Unclassified - Protein with | | | |
| | | | | Unknown Function | | | |
| 299 | GmaAffx.59014.1.S1_s_at | 0.386 | AW733977 | No Homology to Known Proteins | | | |
| 300 | Gma.8946.1.S1_at | 0.386 | BQ453813 | Transcription | Transcription | NAC Domain | |
| | | | | | Factor | | |
| 301 | Gma.2475.1.S1_at | 0.384 | BM522941 | Cell Structure | | | |
| 302 | Gma.6730.1.S1_at | 0.384 | BE211440 | Metabolism | | | UDP-glucuronosyl and UDP-glucosyl |
| | | | | | | | transferase |
| 303 | GmaAffx.13912.1.S1_at | 0.384 | BI699258 | Disease & Defense | | | Purple (tartrate-resistant) acid |
| | | | | | | | phosphatase |
| 304 | Gma.5038.2.S1_a_at | 0.384 | CD417606 | Disease & Defense | | | Acyl-CoA synthetase |
| 305 | Gma.12817.1.A1_at | 0.384 | CD405335 | Unclassified - Hypothetical Protein | | | |
| | | | | Supported by cDNA | | | |
| 306 | GmaAffx.31571.1.S1_at | 0.384 | BU551140 | Unclassified - Hypothetical Protein | | | |
| | | | | NOT Supported by cDNA | | | |
| 307 | GmaAffx.28196.2.A1_s_at | 0.382 | CD416781 | Metabolism | | | Long-chain acyl-CoA synthetases (AMP- |
| | | | | | | | forming) |
| 308 | GmaAffx.90585.1.S1_s_at | 0.382 | CF806310 | Protein Destination & Storage | | | Aspartyl protease |
| 309 | GmaAffx.62856.1.S1_at | 0.382 | AW432978 | Signal Transduction | MAPKKK | ZIK | Serine/threonine protein kinase |
| 310 | Gma.8706.1.S1_at | 0.38 | BU762562 | Unclassified - Hypothetical Protein | | | |
| | | | | Supported by cDNA | | | |
| 311 | GmaAffx.39467.1.S1_at | 0.38 | BQ299569 | Unclassified - Hypothetical Protein | | | |
| | | | | NOT Supported by cDNA | | | |
| 312 | GmaAffx.90789.1.S1_s_at | 0.38 | CF806514 | Unclassified - Protein with | | | |
| | | | | Unknown Function | | | |
| 313 | Gma.9828.1.S1_at | 0.38 | AF529301.1 | Metabolism | | | Haloacid dehalogenase-like hydrolase |
| 314 | GmaAffx.93642.1.S1_s_at | 0.379 | CF809367 | Disease & Defense | | | |
| 315 | Gma.4049.1.S1_at | 0.379 | BQ786519 | Signal Transduction | Protein Kinase | | Serine/threonine protein kinase |
| 316 | Gma.5691.1.S1_at | 0.379 | BM522643 | No Homology to Known Proteins | | | |
| 317 | Gma.5578.3.S1_at | 0.379 | AW310572 | Metabolism | Cytochrome | CYP98A | Cytochrome P450 CYP2 subfamily |
| | | | | | P450 | | |
| 318 | GmaAffx.85878.1.S1_at | 0.378 | BF067810 | Intracellular Traffic | | | Aquaporin (major intrinsic protein |
| | | | | | | | family) |
| 319 | GmaAffx.42856.1.S1_at | 0.378 | BM567981 | Transcription | Transcription | Basic Helix- | |
| | | | | | Factor | Loop-Helix | |
| | | | | | | (bHLH) | |
| 320 | Gma.6343.1.S1_at | 0.377 | AI442963 | Unclassified - Hypothetical Protein | | | |
| | | | | NOT Supported by cDNA | | | |
| 321 | GmaAffx.61571.2.S1_at | 0.377 | AI939190 | Unclassified - Hypothetical Protein | | | |
| | | | | NOT Supported by cDNA | | | |
| 322 | Gma.2831.1.S1_s_at | 0.376 | AW349413 | Transcription | Transcription | Zinc Finger | |
| | | | | | Factor | | |
| 323 | Gma.559.1.A1_at | 0.376 | BQ630327 | Protein Destination & Storage | | | |
| 324 | GmaAffx.38558.1.S1_at | 0.375 | CD407094 | Metabolism | | | NADP+-dependent malic enzyme |
| 325 | GmaAffx.2745.2.A1_at | 0.374 | BQ611985 | Unclassified - Hypothetical Protein | | | Acetylglucosaminyltransferase |
| | | | | Supported by cDNA | | | EXT1/exostosin 1 |
| 326 | GmaAffx.91254.1.S1_at | 0.374 | CF806979 | Unclassified - Hypothetical Protein | | | |
| | | | | Supported by cDNA | | | |
| 327 | GmaAffx.46770.1.S1_at | 0.374 | BQ298902 | Transporter | Ion Channel | Putative ligand- | Glutamate-gated kainate-type ion |
| | | | | | Families | gated ion | channel receptor subunit GluR5 and |
| | | | | | | channel subunit | related subunits |
| | | | | | | family | |
| 328 | GmaAffx.90570.1.S1_at | 0.374 | CF806295 | Unclassified - Hypothetical Protein | | | |
| | | | | Supported by cDNA | | | |
| 329 | GmaAffx.64644.1.S1_at | 0.373 | AW348952 | Signal Transduction | Protein Kinase | | Serine/threonine protein kinase |
| 330 | GmaAffx.79121.1.S1_at | 0.373 | CA801737 | Transcription | Transcription | Zinc Finger | CCCH-type Zn-finger protein |
| | | | | | Factor | | |
| 331 | Gma.5578.3.S1_x_at | 0.372 | AW310572 | Metabolism | Cytochrome | CYP98A | Cytochrome P450 CYP2 subfamily |
| | | | | | P450 | | |
| 332 | GmaAffx.92462.1.S1_at | 0.37 | CF808187 | Transcription | Transcription | Zinc Finger | FOG: Predicted E3 ubiquitin ligase |
| | | | | | Factor | | |
| 333 | GmaAffx.61272.1.S1_at | 0.369 | BI972394 | No Homology to Known Proteins | | | |
| 334 | GmaAffx.6008.1.S1_at | 0.369 | BU550119 | Transcription | Transcription | Basic Helix- | |
| | | | | | Factor | Loop-Helix | |
| | | | | | | (bHLH) | |
| 335 | GmaAffx.44716.1.A1_at | 0.368 | BI969342 | Disease & Defense | | | |
| 336 | GmaAffx.2745.1.S1_at | 0.368 | BF009213 | Unclassified - Hypothetical Protein | | | Acetylglucosaminyltransferase |
| | | | | Supported by cDNA | | | EXT1/exostosin 1 |
| 337 | Gma.5104.1.S1_at | 0.368 | BE611202 | Unclassified - Hypothetical Protein | | | |
| | | | | Supported by cDNA | | | |
| 338 | Gma.1332.1.S1_x_at | 0.368 | BI970666 | Secondary Metabolism | Ethylene | | 1-aminocyclopropane-1-carboxylate |
| | | | | | Biosynthesis | | synthase, and related proteins |
| 339 | Gma.10331.1.A1_at | 0.368 | CD402553 | Unclassified - Protein with | | | Macrophage migration inhibitory factor |
| | | | | Unknown Function | | | |
| 340 | GmaAffx.91917.1.S1_s_at | 0.368 | CF807642 | Unclassified - Hypothetical Protein | | | |
| | | | | Supported by cDNA | | | |
| 341 | GmaAffx.4445.1.A1_at | 0.368 | BU547894 | Transcription | Transcription | AP2/EREBP | |
| | | | | | Factor | | |
| 342 | GmaAffx.44471.1.A1_at | 0.367 | CA851833 | Unclassified - Hypothetical Protein | | | |
| | | | | NOT Supported by cDNA | | | |
| 343 | GmaAffx.87493.1.S1_at | 0.366 | AW781752 | Unclassified - Hypothetical Protein | | | |
| | | | | NOT Supported by cDNA | | | |
| 344 | GmaAffx.7771.1.S1_at | 0.366 | BE022913 | Transporter | Inorganic Solute | Putative sulfate | Sulfate/bicarbonate/oxalate exchanger |
| | | | | | Cotransporters | transporter | SAT-1 and related transporters (SLC26 |
| | | | | | | family | family) |
| 345 | GmaAffx.93214.1.S1_at | 0.366 | CF808939 | No Homology to Known Proteins | | | |
| 346 | GmaAffx.36972.1.S1_at | 0.366 | BQ079450 | Unclassified - Hypothetical Protein | | | |
| | | | | NOT Supported by cDNA | | | |
| 347 | Gma.8160.1.S1_at | 0.366 | BI787214 | Unclassified - Hypothetical Protein | | | |
| | | | | NOT Supported by cDNA | | | |
| 348 | GmaAffx.4936.1.S1_at | 0.365 | AW666270 | Transporter | | | Multidrug/pheromone exporter, ABC |
| | | | | | | | superfamily |
| 349 | GmaAffx.23475.1.A1_at | 0.365 | BU548049 | Unclassified - Hypothetical Protein | | | |
| | | | | Supported by cDNA | | | |
| 350 | GmaAffx.68060.2.A1_at | 0.365 | AW396457 | No Homology to Known Proteins | | | |
| 351 | Gma.3511.4.S1_at | 0.364 | BM526875 | Cell Structure | | | |
| 352 | GmaAffx.81399.1.S1_at | 0.364 | AW568772 | Cell Growth & Division | | | |
| 353 | Gma.2342.1.S1_at | 0.363 | Z46953.1 | Transcription | Transcription Factor | Heat Shock | Heat shock transcription factor |
| 354 | Gma.6836.1.A1_at | 0.363 | AW349121 | Unclassified - Hypothetical Protein | | | Predicted pyroglutamyl peptidase |
| | | | | Supported by cDNA | | | |
| 355 | GmaAffx.37477.1.S1_s_at | 0.363 | BQ081355 | Metabolism | | | Branched chain aminotransferase |
| | | | | | | | BCAT1, pyridoxal phosphate enzymes |
| | | | | | | | type IV superfamily |
| 356 | GmaAffx.75740.1.S1_at | 0.363 | BG157373 | Unclassified - Hypothetical Protein | | | |
| | | | | NOT Supported by cDNA | | | |
| 357 | GmaAffx.23295.1.S1_at | 0.362 | BI968028 | Disease & Defense | | | |
| 358 | Gma.5309.1.S1_at | 0.362 | BM092263 | Unclassified - Hypothetical Protein | | | |
| | | | | NOT Supported by cDNA | | | |
| 359 | GmaAffx.25598.1.S1_at | 0.362 | AW311255 | No Homology to Known Proteins | | | |
| 360 | GmaAffx.84261.1.S1_at | 0.362 | BI701092 | Transcription | Transcription | AP2/EREBP | |
| | | | | | Factor | | |
| 361 | Gma.14075.1.S1_at | 0.362 | CD415333 | Protein Destination & Storage | Storage Protein | | Ca2+-independent phospholipase A2 |
| 362 | GmaAffx.82582.1.S1_at | 0.361 | BM139949 | No Homology to Known Proteins | | | |
| 363 | Gma.1188.2.S1_at | 0.36 | BG406072 | Cell Growth & Division | | | |
| 364 | Gma.364.1.A1_at | 0.359 | AW309875 | Unclassified - Hypothetical Protein | | | |
| | | | | Supported by cDNA | | | |
| 365 | GmaAffx.86675.1.S1_at | 0.359 | BI424294 | No Homology to Known Proteins | | | |
| 366 | GmaAffx.5285.1.S1_at | 0.359 | BM523736 | Metabolism | | | |
| 367 | Gma.6213.1.S1_at | 0.358 | CD401652 | Protein Destination & Storage | | | Glutathione S-transferase |
| 368 | GmaAffx.84607.1.S1_at | 0.357 | BF596776 | Unclassified - Hypothetical Protein | | | |
| | | | | Supported by cDNA | | | |
| 369 | GmaAffx.11857.1.A1_at | 0.357 | AW351021 | Unclassified - Hypothetical Protein | | | |
| | | | | Supported by cDNA | | | |
| 370 | GmaAffx.37615.1.S1_at | 0.357 | BQ454150 | No Homology to Known Proteins | | | |
| 371 | GmaAffx.15471.2.S1_at | 0.357 | BI784829 | Transcription | Transcription | MYB | Transcription factor, Myb superfamily |
| | | | | | Factor | | |
| 372 | GmaAffx.43774.1.A1_at | 0.357 | AW349173 | Cell Structure | | | |
| 373 | GmaAffx.37458.2.S1_at | 0.355 | BQ079820 | Unclassified - Hypothetical Protein | | | |
| | | | | NOT Supported by cDNA | | | |
| 374 | Gma.2602.1.S1_at | 0.355 | BU577743 | No Homology to Known Proteins | | | |
| 375 | Gma.4336.1.S1_at | 0.355 | BQ079907 | Unclassified - Hypothetical Protein | | | |
| | | | | NOT Supported by cDNA | | | |
| 376 | Gma.5478.1.S1_at | 0.353 | AW350606 | Metabolism | | | |
| 377 | Gma.10701.1.S1_at | 0.353 | BF596612 | Unclassified - Hypothetical Protein | | | |
| | | | | Supported by cDNA | | | |
| 378 | GmaAffx.37055.1.A1_at | 0.353 | BM142939 | Metabolism | | | UDP-glucuronosyl and UDP-glucosyl |
| | | | | | | | transferase |
| 379 | Gma.11119.2.S1_s_at | 0.353 | CF920743 | Disease & Defense | | | |
| 380 | Gma.8331.1.S1_at | 0.352 | BE659403 | Metabolism | | | Aldehyde dehydrogenase |
| 381 | Gma.11277.1.S1_at | 0.351 | CA802310 | Protein Destination & Storage | | | |
| 382 | Gma.10365.1.S1_at | 0.351 | BG725377 | Unclassified - Hypothetical Protein | | | |
| | | | | Supported by cDNA | | | |
| 383 | GmaAffx.70210.1.S1_at | 0.351 | BI699931 | Transcription | Transcription | Unclassified | |
| | | | | | Factor | | |
| 384 | Gma.14567.1.S1_at | 0.35 | BE824123 | Disease & Defense | | | |
| 385 | Gma.14325.1.S1_at | 0.35 | BI700686 | Disease & Defense | | | Ca2+-independent phospholipase A2 |
| 386 | Gma.4372.1.S1_at | 0.35 | AI443505 | Secondary Metabolism | | | Reductases with broad range of substrate |
| | | | | | | | specificities |
| 387 | GmaAffx.66676.1.S1_at | 0.35 | BQ610288 | Unclassified - Protein with | | | |
| | | | | Unknown Function | | | |
| 388 | Gma.13418.1.S1_at | 0.35 | BM143894 | Transporter | | | |
| 389 | GmaAffx.37574.1.S1_s_at | 0.349 | BQ298504 | Secondary Metabolism | | | Hydroxyindole-O-methyltransferase and |
| | | | | | | | related SAM-dependent |
| | | | | | | | methyltransferases |
| 390 | Gma.382.1.A1_at | 0.349 | AW349068 | Metabolism | | | Arylacetamide deacetylase |
| 391 | Gma.752.1.A1_at | 0.349 | AW432463 | Transcription | Transcription | Basic Helix- | |
| | | | | | Factor | Loop-Helix | |
| | | | | | | (bHLH) | |
| 392 | GmaAffx.32765.1.S1_at | 0.348 | BE024072 | No Homology to Known Proteins | | | |
| 393 | Gma.10756.1.S1_s_at | 0.348 | BE822092 | Unclassified - Hypothetical Protein | | | |
| | | | | Supported by cDNA | | | |
| 394 | GmaAffx.23276.1.S1_at | 0.347 | AI856198 | Transporter | | | Amino acid transporters |
| 395 | GmaAffx.50673.2.S1_s_at | 0.347 | BI787147 | Transcription | Transcription | NAC Domain | |
| | | | | | Factor | | |
| 396 | GmaAffx.24359.1.A1_at | 0.346 | BU544840 | Metabolism | | | |
| 397 | GmaAffx.91561.1.S1_s_at | 0.346 | CF807286 | Transporter | | | Apolipoprotein D/Lipocalin |
| 398 | Gma.5084.1.S1_at | 0.346 | AW309848 | Protein Destination & Storage | Storage Protein | | |
| 399 | Gma.9056.1.S1_at | 0.343 | CA819787 | Metabolism | | | Acyl-CoA-binding protein |
| 400 | Gma.10612.1.S1_s_at | 0.343 | BG650314 | No Homology to Known Proteins | | | |
| 401 | GmaAffx.1301.52.S1_at | 0.343 | BG510366 | Unclassified - Hypothetical Protein | | | |
| | | | | Supported by cDNA | | | |
| 402 | GmaAffx.65217.1.A1_at | 0.343 | BU543988 | Metabolism | | | Acyl-CoA-binding protein |
| 403 | Gma.17729.1.S1_s_at | 0.343 | CD393936 | No Homology to Known Proteins | | | |
| 404 | GmaAffx.83964.1.S1_at | 0.342 | BU082747 | Unclassified - Protein with | | | Protein involved in mRNA turnover and |
| | | | | Unknown Function | | | stability |
| 405 | GmaAffx.33408.1.S1_at | 0.342 | BU547044 | No Homology to Known Proteins | | | |
| 406 | Gma.1137.1.S1_x_at | 0.342 | BF067710 | Metabolism | | | |
| 407 | GmaAffx.46681.1.S1_at | 0.342 | BQ080794 | Unclassified - Hypothetical Protein | | | |
| | | | | Supported by cDNA | | | |
| 408 | Gma.5256.1.A1_at | 0.341 | CD408042 | No Homology to Known Proteins | | | |
| 409 | Gma.11277.1.S1_x_at | 0.341 | CA802310 | Protein Destination & Storage | | | |
| 410 | Gma.5255.1.S1_at | 0.34 | AI736533 | Unclassified - Hypothetical Protein | | | |
| | | | | Supported by cDNA | | | |
| 411 | Gma.12902.1.A1_at | 0.34 | CD395017 | Metabolism | | | Hydroxyindole-O-methyltransferase and |
| | | | | | | | related SAM-dependent |
| | | | | | | | methyltransferases |
| 412 | Gma.3917.1.S1_at | 0.34 | BE658782 | Cell Structure | | | Pectin acetylesterase and similar |
| | | | | | | | proteins |
| 413 | Gma.9907.1.S1_at | 0.339 | AW309518 | Transcription | Transcription | Basic Helix- | |
| | | | | | Factor | Loop-Helix | |
| | | | | | | (bHLH) | |
| 414 | GmaAffx.29254.1.S1_at | 0.338 | AW760361 | Transcription | Transcription | Basic Helix- | |
| | | | | | Factor | Loop-Helix | |
| | | | | | | (bHLH) | |
| 415 | Gma.5578.4.S1_a_at | 0.338 | BE023932 | Metabolism | Cytochrome | CYP98A | Cytochrome P450 CYP2 subfamily |
| | | | | | P450 | | |
| 416 | GmaAffx.37458.1.S1_at | 0.338 | BU549419 | Unclassified - Protein with | | | |
| | | | | Unknown Function | | | |
| 417 | Gma.10824.1.S1_at | 0.338 | BE824257 | Unclassified - Hypothetical Protein | | | Magnesium transporters: CorA family |
| | | | | Supported by cDNA | | | |
| 418 | GmaAffx.93189.1.S1_at | 0.338 | CF808914 | Metabolism | | | Carbon-nitrogen hydrolase |
| 419 | Gma.4222.1.S1_at | 0.337 | BU083971 | Unclassified - Hypothetical Protein | | | Arylacetamide deacetylase |
| | | | | NOT Supported by cDNA | | | |
| 420 | GmaAffx.64075.1.S1_at | 0.337 | BI700868 | Unclassified - Hypothetical Protein | | | |
| | | | | Supported by cDNA | | | |
| 421 | GmaAffx.91328.1.S1_at | 0.336 | CF807053 | Unclassified - Hypothetical Protein | | | Leucine rich repeat proteins, some |
| | | | | Supported by cDNA | | | proteins contain F-box |
| 422 | Gma.10827.2.S1_s_at | 0.335 | BQ612452 | Protein Destination & Storage | Storage Protein | | |
| 423 | GmaAffx.92070.1.S1_at | 0.335 | CF807049 | Cell Structure | | | O-methyltransferase |
| 424 | GmaAffx.10908.1.S1_at | 0.335 | BE822295 | Transcription | Transcription | Homeodomain | |
| | | | | | Factor | | |
| 425 | Gma.17610.1.S1_x_at | 0.334 | U50075.1 | Metabolism | | | |
| 426 | Gma.14361.1.A1_at | 0.333 | CD409843 | No Homology to Known Proteins | | | |
| 427 | Gma.336.1.A1_at | 0.333 | AW310815 | Metabolism | Sulfur | | |
| | | | | | Metabolism | | |
| 428 | GmaAffx.41990.1.S1_at | 0.332 | CA936708 | No Homology to Known Proteins | | | |
| 429 | Gma.6782.2.S1_a_at | 0.332 | AW156813 | Unclassified - Hypothetical Protein | | | |
| | | | | Supported by cDNA | | | |
| 430 | GmaAffx.93182.1.S1_s_at | 0.332 | CF808907 | Secondary Metabolism | Ethylene | | Iron/ascorbate family oxidoreductases |
| | | | | | Biosynthesis | | |
| 431 | GmaAffx.62364.1.S1_at | 0.331 | AW733764 | Protein Destination & Storage | | | Dipeptidyl aminopeptidase |
| 432 | Gma.8312.1.A1_at | 0.331 | AW310254 | Unclassified - Protein with | | | |
| | | | | Unknown Function | | | |
| 433 | Gma.4093.1.S1_at | 0.33 | BI700897 | Transporter | | | |
| 434 | GmaAffx.17098.1.S1_at | 0.33 | BM084976 | Disease & Defense | | | |
| 435 | GmaAffx.33778.1.S1_at | 0.329 | CA819035 | Protein Destination & Storage | Storage Protein | | |
| 436 | Gma.6550.3.S1_s_at | 0.329 | AW234450 | Metabolism | | | Hydroxyindole-O-methyltransferase and |
| | | | | | | | related SAM-dependent |
| | | | | | | | methyltransferases |
| 437 | Gma.1622.1.A1_s_at | 0.329 | BU090523 | Signal Transduction | Protein Kinase | | FOG: Transposon-encoded proteins with |
| | | | | | | | TYA, reverse transcriptase, integrase |
| | | | | | | | domains in various combinations |
| 438 | Gma.7107.1.S1_at | 0.329 | AI855972 | Unclassified - Protein with | | | |
| | | | | Unknown Function | | | |
| 439 | Gma.4873.1.S1_at | 0.328 | BI969532 | Signal Transduction | Protein Kinase | | Serine/threonine protein kinase |
| 440 | GmaAffx.2716.1.A1_at | 0.327 | BU547858 | Transcription | Transcription | Zinc Finger | Protein involved in mRNA turnover and |
| | | | | | Factor | | stability |
| 441 | Gma.4897.1.S1_at | 0.326 | BI969641 | Unclassified - Protein with | | | Thioredoxin, nucleoredoxin and related |
| | | | | Unknown Function | | | proteins |
| 442 | GmaAffx.93553.1.S1_s_at | 0.325 | CF809278 | Metabolism | | | |
| 443 | GmaAffx.91254.1.S1_x_at | 0.324 | CF806979 | Unclassified - Hypothetical Protein | | | |
| | | | | Supported by cDNA | | | |
| 444 | GmaAffx.63553.1.S1_at | 0.324 | BG352380 | Energy | | | Cytochrome b |
| 445 | GmaAffx.93189.1.S1_s_at | 0.324 | CF808914 | Metabolism | | | Carbon-nitrogen hydrolase |
| 446 | GmaAffx.47819.1.A1_s_at | 0.324 | CD416142 | Unclassified - Hypothetical Protein | | | Predicted mitochondrial ribosomal |
| | | | | Supported by cDNA | | | protein L23 |
| 447 | Gma.5097.1.A1_at | 0.323 | BI969945 | Unclassified - Hypothetical Protein | | | |
| | | | | Supported by cDNA | | | |
| 448 | Gma.1895.1.A1_at | 0.323 | AW309584 | Protein Destination & Storage | | | ER lumen protein retaining receptor |
| 449 | Gma.15359.2.S1_a_at | 0.322 | AW781747 | Intracellular Traffic | | | Predicted Ca2+-dependent phospholipid- |
| | | | | | | | binding protein |
| 450 | GmaAffx.84566.1.S1_x_at | 0.322 | BI786849 | Transcription | Transcription | MYB | Transcription factor, Myb superfamily |
| | | | | | Factor | | |
| 451 | Gma.12211.2.S1_at | 0.322 | BQ299492 | Disease & Defense | | | |
| 452 | GmaAffx.70959.1.S1_at | 0.32 | BE611219 | Unclassified - Hypothetical Protein | | | Glucose-6-phosphate/phosphate and |
| | | | | NOT Supported by cDNA | | | phosphoenolpyruvate/phosphate |
| | | | | | | | antiporter |
| 453 | Gma.8484.1.S1_at | 0.32 | AF197946.1 | Signal Transduction | Protein Kinase | | |
| 454 | Gma.6213.1.S1_s_at | 0.32 | CD401652 | Protein Destination & Storage | | | Glutathione S-transferase |
| 455 | GmaAffx.24350.1.A1_at | 0.32 | BU544060 | No Homology to Known Proteins | | | |
| 456 | GmaAffx.81287.1.S1_at | 0.319 | BG789983 | Secondary Metabolism | | | Hydroxyindole-O-methyltransferase and |
| | | | | | | | related SAM-dependent |
| | | | | | | | methyltransferases |
| 457 | Gma.13361.1.S1_at | 0.318 | AW309932 | Unclassified - Protein with | | | |
| | | | | Unknown Function | | | |
| 458 | Gma.2712.1.S1_at | 0.318 | AJ003246.1 | Secondary Metabolism | | | Flavonol reductase/cinnamoyl-CoA |
| | | | | | | | reductase |
| 459 | GmaAffx.89867.1.A1_s_at | 0.317 | CK606556 | Metabolism | | | Predicted PhzC/PhzF-type epimerase |
| 460 | Gma.2302.1.S1_x_at | 0.317 | AW307490 | Disease & Defense | | | Cysteine proteinase Cathepsin L |
| 461 | GmaAffx.72986.1.S1_at | 0.315 | BI317536 | Metabolism | | | Inositol polyphosphate 5-phosphatase |
| | | | | | | | and related proteins |
| 462 | GmaAffx.80098.1.S1_at | 0.315 | BG042861 | Metabolism | | | Arylacetamide deacetylase |
| 463 | Gma.16940.1.S1_at | 0.315 | AW310039 | Transcription | Transcription | Homeodomain | Transcription factor MEIS1 and related |
| | | | | | Factor | | HOX domain proteins |
| 464 | Gma.10950.1.S1_at | 0.315 | BI699798 | Unclassified - Hypothetical Protein | | | |
| | | | | Supported by cDNA | | | |
| 465 | Gma.157.1.S1_at | 0.314 | D86351.1 | Metabolism | Cytochrome | CYP93D | Cytochrome P450 CYP2 subfamily |
| | | | | | P450 | | |
| 466 | GmaAffx.26967.1.S1_at | 0.314 | BE022958 | Unclassified - Hypothetical Protein | | | |
| | | | | Supported by cDNA | | | |
| 467 | GmaAffx.28539.1.S1_at | 0.313 | BQ080184 | Disease & Defense | | | Ca2+-independent phospholipase A2 |
| 468 | GmaAffx.9127.1.S1_s_at | 0.313 | BI317582 | Transcription | Transcription | Zinc Finger | FOG: Predicted E3 ubiquitin ligase |
| | | | | | Factor | | |
| 469 | GmaAffx.3706.1.S1_at | 0.313 | BE821261 | Unclassified - Hypothetical Protein NOT | | | HGG motif-containing thioesterase |
| | | | | Supported by cDNA | | | |
| 470 | Gma.4526.1.S1_at | 0.312 | BI967711 | Transcription | Transcription | Zinc Finger | FOG: Zn-finger |
| | | | | | Factor | | |
| 471 | Gma.1704.1.S1_at | 0.312 | AW349971 | Metabolism | | | Glutaredoxin and related proteins |
| 472 | GmaAffx.82564.1.S1_s_at | 0.312 | BG047418 | No Homology to Known Proteins | | | |
| 473 | Gma.9622.1.S1_x_at | 0.311 | BQ610840 | Metabolism | | | Hydroxyindole-O-methyltransferase and |
| | | | | | | | related SAM-dependent |
| | | | | | | | methyltransferases |
| 474 | Gma.9205.1.S1_at | 0.31 | BM528605 | Unclassified - Hypothetical Protein | | | |
| | | | | Supported by cDNA | | | |
| 475 | Gma.2776.1.S1_at | 0.31 | BI968513 | Secondary Metabolism | Ethylene | | Iron/ascorbate family oxidoreductases |
| | | | | | Biosynthesis | | |
| 476 | Gma.6550.2.S1_x_at | 0.309 | BM086044 | Metabolism | | | Hydroxyindole-O-methyltransferase and |
| | | | | | | | related SAM-dependent |
| | | | | | | | methyltransferases |
| 477 | Gma.3581.2.S1_x_at | 0.309 | CD398783 | Unclassified - Hypothetical Protein | | | Ca2+-dependent lipid-binding protein |
| | | | | NOT Supported by cDNA | | | CLB1/vesicle protein |
| | | | | | | | vp115/Granuphilin A, contains C2 |
| | | | | | | | domain |
| 478 | GmaAffx.75594.1.S1_at | 0.309 | AW307139 | Secondary Metabolism | | | Multicopper oxidases |
| 479 | GmaAffx.14030.1.S1_at | 0.308 | BE021503 | No Homology to Known Proteins | | | |
| 480 | GmaAffx.87101.1.S1_at | 0.308 | BM309451 | No Homology to Known Proteins | | | |
| 481 | GmaAffx.55362.1.S1_at | 0.308 | BE475283 | Unclassified - Hypothetical Protein | | | |
| | | | | Supported by cDNA | | | Reticulon |
| 482 | Gma.79.4.S1_s_at | 0.308 | AI443819 | Unclassified - Hypothetical Protein | | | |
| | | | | Supported by cDNA | | | |
| 483 | Gma.8556.1.S1_at | 0.308 | BU764573 | Transporter | | | Amino acid transporters |
| 484 | GmaAffx.82195.1.S1_at | 0.308 | BM528489 | Unclassified - Hypothetical Protein | | | |
| | | | | NOT Supported by cDNA | | | |
| 485 | GmaAffx.90515.1.S1_s_at | 0.306 | CF806240 | Unclassified - Hypothetical Protein | | | Predicted pyroglutamyl peptidase |
| | | | | Supported by cDNA | | | |
| 486 | Gma.4578.1.S1_s_at | 0.306 | BE802809 | Transporter | | | Plasma membrane H+-transporting |
| | | | | | | | ATPase |
| 487 | GmaAffx.47739.1.S1_at | 0.305 | BM522736 | Unclassified - Protein with | | | |
| | | | | Unknown Function | | | |
| 488 | Gma.3161.2.S1_at | 0.305 | CD400025 | Secondary Metabolism | | | Iron/ascorbate family oxidoreductases |
| 489 | Gma.3976.1.S1_at | 0.304 | BI972268 | No Homology to Known Proteins | | | |
| 490 | GmaAffx.72479.1.S1_at | 0.303 | BG043825 | Transcription | Transcription | AP2/EREBP | Uncharacterized conserved protein |
| | | | | | Factor | | |
| 491 | Gma.6550.2.S1_s_at | 0.302 | BM086044 | Metabolism | | | Hydroxyindole-O-methyltransferase and |
| | | | | | | | related SAM-dependent |
| | | | | | | | methyltransferases |
| 492 | GmaAffx.57748.1.S1_at | 0.302 | AW306274 | No Homology to Known Proteins | | | |
| 493 | Gma.3751.1.S1_at | 0.301 | AW350303 | Unclassified - Hypothetical Protein | | | |
| | | | | NOT Supported by cDNA | | | |
| 494 | GmaAffx.16207.1.S1_at | 0.301 | BE821188 | Cell Structure | | | Beta-glucosidase, lactase |
| | | | | | | | phlorizinhydrolase, and related proteins |
| 495 | Gma.15359.1.S1_x_at | 0.3 | BM271008 | Unclassified - Protein with | | | Predicted Ca2+-dependent phospholipid- |
| | | | | Unknown Function | | | binding protein |
| 496 | GmaAffx.60672.1.S1_at | 0.299 | BU080558 | No Homology to Known Proteins | | | |
| 497 | Gma.6462.2.S1_a_at | 0.299 | BE555263 | Unclassified - Hypothetical Protein | | | |
| | | | | NOT Supported by cDNA | | | |
| 498 | GmaAffx.9926.1.A1_at | 0.299 | CD411291 | No Homology to Known Proteins | | | |
| 499 | GmaAffx.78297.1.S1_at | 0.299 | BM731185 | Unclassified - Hypothetical Protein | | | FOG: Predicted E3 ubiquitin ligase |
| | | | | Supported by cDNA | | | |
| 500 | Gma.2302.1.S1_at | 0.299 | AW307490 | Disease & Defense | | | Cysteine proteinase Cathepsin L |
| 501 | Gma.73.1.S1_at | 0.298 | AF098662.1 | Protein Destination & Storage | | | |
| 502 | Gma.6151.1.S1_at | 0.298 | BQ611806 | Metabolism | | | Flavin-containing monooxygenase |
| 503 | Gma.17610.1.S1_at | 0.298 | U50075.1 | Metabolism | | | |
| 504 | GmaAffx.89060.1.S1_at | 0.296 | CK605664 | Metabolism | Cytochrome | CYP93D | Cytochrome P450 CYP2 subfamily |
| | | | | | P450 | | |
| 505 | GmaAffx.89772.10.A1_s_at | 0.296 | CK606097 | Disease & Defense | | | |
| 506 | GmaAffx.92112.1.S1_s_at | 0.295 | CF807837 | No Homology to Known Proteins | | | |
| 507 | Gma.6623.1.S1_at | 0.294 | BI702268 | Unclassified - Protein with | | | 1,4-benzoquinone reductase-like; Trp |
| | | | | Unknown Function | | | repressor binding protein-like/protoplast- |
| | | | | | | | secreted protein |
| 508 | GmaAffx.74300.1.S1_at | 0.294 | BU080963 | Disease & Defense | | | |
| 509 | GmaAffx.61311.1.S1_at | 0.294 | CA936951 | Transporter | | | Amino acid transporters |
| 510 | Gma.3963.2.S1_at | 0.293 | BE608192 | Unclassified - Hypothetical Protein | | | Zn-finger protein |
| | | | | NOT Supported by cDNA | | | |
| 511 | Gma.6640.1.S1_at | 0.292 | BQ628278 | Disease & Defense | | | |
| 512 | Gma.9236.1.S1_at | 0.291 | BG405642 | Metabolism | Amino Acid | | Spermidine synthase |
| | | | | | Biosynthesis/Metabolism | | |
| 513 | Gma.5733.1.S1_at | 0.291 | CD402671 | Signal Transduction | Calcium Binding | | Ca2+-binding protein |
| | | | | | | | (centrin/caltractin), EF-Hand |
| | | | | | | | superfamily protein |
| 514 | GmaAffx.18953.1.S1_at | 0.291 | BM178798 | No Homology to Known Proteins | | | |
| 515 | GmaAffx.90703.1.A1_at | 0.29 | CF809087 | Disease & Defense | | | |
| 516 | GmaAffx.79390.2.S1_at | 0.29 | AW597383 | Transcription | Transcription | Basic Leucine | FOG: Armadillo/beta-catenin-like |
| | | | | | Factor | Zipper (bZ1P) | repeats |
| 517 | Gma.6310.1.S1_at | 0.29 | BQ612831 | Signal Transduction | MAPKKK | MEKK | MEKK and related serine/threonine |
| | | | | | | | protein kinases |
| 518 | Gma.15605.1.S1_at | 0.288 | BM143798 | Transcription | Transcription Factor | AP2/EREBP | |
| 519 | Gma.17835.1.S1_at | 0.288 | BE822840 | Metabolism | | | |
| 520 | Gma.8394.1.S1_at | 0.288 | CD392332 | Disease & Defense | | | Prohibitins and stomatins of the PID |
| | | | | | | | superfamily |
| 521 | Gma.6630.1.S1_s_at | 0.288 | BG652851 | Unclassified - Hypothetical Protein | | | |
| | | | | Supported by cDNA | | | |
| 522 | Gma.2388.1.S1_at | 0.288 | CD416539 | No Homology to Known Proteins | | | |
| 523 | GmaAffx.727.1.S1_at | 0.287 | BE804565 | Unclassified - Protein with | | | Ubiquinone biosynthesis |
| | | | | Unknown Function | | | methyltransferase COQ5 |
| 524 | Gma.13000.1.S1_at | 0.287 | CD415111 | Unclassified - Hypothetical Protein | | | |
| | | | | Supported by cDNA | | | |
| 525 | Gma.5578.1.S1_x_at | 0.287 | BE659192 | Metabolism | Cytochrome | CYP98A | Cytochrome P450 CYP2 subfamily |
| | | | | | P450 | | |
| 526 | GmaAffx.93607.1.S1_s_at | 0.286 | CF809332 | Unclassified - Protein with | | | |
| | | | | Unknown Function | | | |
| 527 | Gma.11277.2.S1_a_at | 0.286 | BI701238 | Unclassified - Hypothetical Protein | | | |
| | | | | NOT Supported by cDNA | | | |
| 528 | GmaAffx.478.2.S1_at | 0.285 | BU763963 | Transcription | Transcription | Homeodomain | Transcription factor MEIS1 and related |
| | | | | | Factor | | HOX domain proteins |
| 529 | Gma.3345.1.S1_at | 0.285 | AW394524 | Metabolism | | | Alcohol dehydrogenase, class III |
| 530 | GmaAffx.84338.1.S1_at | 0.285 | AI856789 | Unclassified - Hypothetical Protein | | | Predicted E3 ubiquitin ligase |
| | | | | Supported by cDNA | | | |
| 531 | Gma.6187.1.S1_at | 0.283 | BF324343 | No Homology to Known Proteins | | | |
| 532 | Gma.12211.1.S1_at | 0.283 | AW394548 | Disease & Defense | | | |
| 533 | Gma.4822.1.S1_at | 0.283 | BI969249 | Disease & Defense | | | |
| 534 | GmaAffx.32328.1.A1_at | 0.283 | BI968685 | No Homology to Known Proteins | | | |
| 535 | Gma.3963.1.S1_at | 0.283 | AW202007 | Unclassified - Hypothetical Protein | | | Zn-finger protein |
| | | | | NOT Supported by cDNA | | | |
| 536 | GmaAffx.56606.1.S1_at | 0.283 | BG507644 | Secondary Metabolism | | | Multicopper oxidases |
| 537 | Gma.850.1.S1_at | 0.281 | BE822050 | Unclassified - Hypothetical Protein | | | |
| | | | | NOT Supported by cDNA | | | |
| 538 | Gma.5634.1.S1_s_at | 0.28 | BE806891 | Secondary Metabolism | | | Steroid reductase |
| 539 | Gma.1332.1.S1_s_at | 0.279 | BI970666 | Secondary Metabolism | Ethylene | | 1-aminocyclopropane-1-carboxylate |
| | | | | | Biosynthesis | | synthase, and related proteins |
| 540 | Gma.1068.1.S1_at | 0.279 | L19360.1 | Signal Transduction | Protein Kinase | | Serine/threonine protein kinase |
| 541 | Gma.2590.1.A1_s_at | 0.279 | CD406617 | Disease & Defense | | | |
| 542 | Gma.6047.1.S1_s_at | 0.278 | BQ611159 | Unclassified - Protein with | | | |
| | | | | Unknown Function | | | |
| 543 | Gma.1716.1.S1_at | 0.278 | AW156801 | Unclassified - Hypothetical Protein | | | |
| | | | | Supported by cDNA | | | |
| 544 | GmaAffx.50646.1.A1_at | 0.277 | AW310451 | Unclassified - Hypothetical Protein | | | Molybdenum cofactor sulfurase |
| | | | | Supported by cDNA | | | |
| 545 | Gma.3591.2.S1_at | 0.277 | BE473658 | Secondary Metabolism | Ethylene | | Iron/ascorbate family oxidoreductases |
| | | | | | Biosynthesis | | |
| 546 | Gma.17015.1.S1_at | 0.275 | AW733363 | No Homology to Known Proteins | | | |
| 547 | GmaAffx.80830.1.A1_at | 0.275 | BU549591 | Unclassified - Hypothetical Protein | | | |
| | | | | Supported by cDNA | | | |
| 548 | Gma.7494.1.S1_at | 0.275 | BU548105 | Signal Transduction | Protein | | Serine/threonine protein phosphatase |
| | | | | | Phosphatase | | |
| 549 | Gma.4593.1.S1_a_at | 0.275 | CD396298 | Energy | | | |
| 550 | GmaAffx.85528.1.S1_at | 0.274 | BE608070 | Unclassified - Hypothetical Protein | | | FOG: PPR repeat |
| | | | | Supported by cDNA | | | |
| 551 | Gma.6623.2.S1_s_at | 0.273 | BQ629284 | Unclassified - Protein with | | | 1,4-benzoquinone reductase-like; Trp |
| | | | | Unknown Function | | | repressor binding protein-like/protoplast- |
| | | | | | | | secreted protein |
| 552 | GmaAffx.92568.1.S1_at | 0.272 | CF808293 | Metabolism | | | Acyl-CoA-binding protein |
| 553 | Gma.9534.1.S1_at | 0.272 | CA802874 | Disease & Defense | | | |
| 554 | Gma.11284.1.S1_at | 0.272 | AW153035 | Disease & Defense | | | |
| 555 | Gma.6999.1.S1_x_at | 0.271 | AF529303.1 | Disease & Defense | | | |
| 556 | Gma.6550.1.A1_at | 0.271 | CD402638 | Metabolism | | | Hydroxyindole-O-methyltransferase and |
| | | | | | | | related SAM-dependent |
| | | | | | | | methyltransferases |
| 557 | Gma.15155.2.S1_at | 0.27 | BG789619 | Unclassified - Hypothetical Protein | | | |
| | | | | Supported by cDNA | | | |
| 558 | Gma.426.1.A1_at | 0.27 | AI442768 | Transcription | Transcription | Zinc Finger | |
| | | | | | Factor | | |
| 559 | Gma.17300.1.A1_at | 0.27 | AW350946 | Unclassified - Hypothetical Protein | | | |
| | | | | NOT Supported by cDNA | | | |
| 560 | Gma.4093.2.S1_at | 0.267 | BQ081460 | Transporter | | | |
| 561 | Gma.11277.2.S1_at | 0.267 | BI701238 | Unclassified - Hypothetical Protein | | | |
| | | | | NOT Supported by cDNA | | | |
| 562 | GmaAffx.91266.1.S1_s_at | 0.267 | CF806991 | Unclassified - Hypothetical Protein | | | |
| | | | | Supported by cDNA | | | |
| 563 | Gma.1137.1.S1_a_at | 0.266 | BF067710 | Metabolism | | | |
| 564 | GmaAffx.45805.2.S1_at | 0.265 | CA784263 | Metabolism | | | Hydroxyindole-O-methyltransferase and |
| | | | | | | | related SAM-dependent |
| | | | | | | | methyltransferases |
| 565 | GmaAffx.84566.1.S1_at | 0.265 | BI786849 | Transcription | Transcription | MYB | Transcription factor, Myb superfamily |
| | | | | | Factor | | |
| 566 | GmaAffx.4888.1.A1_at | 0.264 | BI967635 | No Homology to Known Proteins | | | |
| 567 | Gma.14897.2.S1_at | 0.263 | BQ785154 | Unclassified - Protein with | | | Dihydroorotase and related enzymes |
| | | | | Unknown Function | | | |
| 568 | Gma.8515.1.S1_at | 0.263 | AF243372.1 | Protein Destination & Storage | | | Glutathione S-transferase |
| 569 | GmaAffx.53914.1.A1_at | 0.262 | BE822062 | No Homology to Known Proteins | | | |
| 570 | Gma.1758.1.S1_at | 0.262 | CD402046 | Unclassified - Protein with | | | |
| | | | | Unknown Function | | | |
| 571 | GmaAffx.86927.1.S1_at | 0.261 | CA937162 | No Homology to Known Proteins | | | |
| 572 | GmaAffx.60958.1.S1_at | 0.261 | BG551038 | Transcription | Transcription | Basic Helix- | |
| | | | | | Factor | Loop-Helix | |
| | | | | | | (bHLH) | |
| 573 | Gma.3893.3.S1_at | 0.261 | BG651854 | Unclassified - Hypothetical Protein | | | |
| | | | | Supported by cDNA | | | |
| 574 | GmaAffx.86247.1.S1_s_at | 0.26 | BG238674 | Unclassified - Hypothetical Protein | | | |
| | | | | Supported by cDNA | | | |
| 575 | Gma.15359.1.S1_a_at | 0.26 | BM271008 | Unclassified - Protein with | | | Predicted Ca2+-dependent phospholipid- |
| | | | | Unknown Function | | | binding protein |
| 576 | Gma.5578.3.S1_a_at | 0.259 | AW310572 | Metabolism | Cytochrome | CYP98A | Cytochrome P450 CYP2 subfamily |
| | | | | | P450 | | |
| 577 | GmaAffx.76821.1.S1_at | 0.259 | BG238158 | Unclassified - Protein with | Hormone | | |
| | | | | Unknown Function | Induced | | |
| 578 | GmaAffx.91328.1.S1_s_at | 0.258 | CF807053 | Unclassified - Hypothetical Protein | | | Leucine rich repeat proteins, some |
| | | | | Supported by cDNA | | | proteins contain F-box |
| 579 | GmaAffx.50613.1.A1_at | 0.258 | BI967418 | No Homology to Known Proteins | | | |
| 580 | Gma.7102.1.S1_a_at | 0.258 | AW309275 | Unclassified - Hypothetical Protein | | | Leucine rich repeat proteins, some |
| | | | | Supported by cDNA | | | proteins contain F-box |
| 581 | GmaAffx.20108.1.S1_at | 0.256 | CA801220 | Unclassified - Hypothetical Protein | | | |
| | | | | Supported by cDNA | | | |
| 582 | Gma.5634.1.S1_at | 0.256 | BE806891 | Secondary Metabolism | | | Steroid reductase |
| 583 | GmaAffx.92148.1.S1_s_at | 0.255 | CF807873 | Unclassified - Hypothetical Protein | | | |
| | | | | NOT Supported by cDNA | | | |
| 584 | Gma.2080.2.S1_a_at | 0.255 | BM520916 | Secondary Metabolism | | | |
| 585 | GmaAffx.92136.1.S1_s_at | 0.254 | CF807451 | Signal Transduction | Protein Kinase | | Mannosyltransferase |
| 586 | Gma.2102.1.S1_s_at | 0.253 | BM523392 | Disease & Defense | | | |
| 587 | GmaAffx.83172.1.S1_at | 0.253 | BQ610576 | Unclassified - Protein with | | | |
| | | | | Unknown Function | | | |
| 588 | Gma.6730.1.S1_s_at | 0.252 | BE211440 | Metabolism | | | UDP-glucuronosyl and UDP-glucosyl |
| | | | | | | | transferase |
| 589 | GmaAffx.85662.1.S1_at | 0.252 | BG404902 | Unclassified - Protein with | | | Predicted membrane protein, contains |
| | | | | Unknown Function | | | DoH and Cytochrome b-561/ferric |
| | | | | | | | reductase transmembrane domains |
| 590 | Gma.7836.1.A1_at | 0.251 | BU549650 | Unclassified - Hypothetical Protein | | | |
| | | | | NOT Supported by cDNA | | | |
| 591 | Gma.11277.1.S1_a_at | 0.251 | CA802310 | Protein Destination & Storage | | | |
| 592 | Gma.9086.2.S1_at | 0.251 | BE555169 | Cell Growth & Division | | | |
| 593 | GmaAffx.50811.2.S1_at | 0.251 | BG725712 | Transcription | Transcription | NAC Domain | |
| | | | | | Factor | | |
| 594 | Gma.8365.1.S1_at | 0.25 | CD396813 | Unclassified - Hypothetical Protein | | | |
| | | | | NOT Supported by cDNA | | | |
| 595 | Gma.9856.1.S1_at | 0.25 | BG650352 | Signal Transduction | Protein Kinase | | Serine/threonine protein kinase |
| 596 | Gma.8113.1.A1_at | 0.249 | BU548342 | No Homology to Known Proteins | | | |
| 597 | GmaAffx.5743.1.S1_at | 0.247 | BQ628343 | Unclassified - Hypothetical Protein | | | FOG: Predicted E3 ubiquitin ligase |
| | | | | Supported by cDNA | | | |
| 598 | Gma.6719.2.S1_at | 0.247 | BG726773 | No Homology to Known Proteins | | | |
| 599 | Gma.358.1.S1_at | 0.247 | AW310270 | Metabolism | | | Arylacetamide deacetylase |
| 600 | GmaAffx.2026.1.A1_at | 0.246 | BU550139 | Transcription | Transcription | NAC Domain | |
| | | | | | Factor | | |
| 601 | Gma.10342.1.S1_at | 0.246 | BE058741 | Unclassified - Hypothetical Protein | | | |
| | | | | Supported by cDNA | | | |
| 602 | GmaAffx.61872.1.S1_at | 0.245 | AW102386 | Signal Transduction | Protein Kinase | | |
| 603 | Gma.2439.1.S1_at | 0.244 | BI972053 | Metabolism | | | Predicted PhzC/PhzF-type epimerase |
| 604 | GmaAffx.24102.1.S1_at | 0.244 | BQ453911 | Unclassified - Hypothetical Protein | | | |
| | | | | Supported by cDNA | | | |
| 605 | GmaAffx.90395.1.A1_s_at | 0.244 | CF807499 | Unclassified - Protein with | | | |
| | | | | Unknown Function | | | |
| 606 | GmaAffx.15613.1.S1_at | 0.243 | AI939073 | Unclassified - Hypothetical Protein | | | |
| | | | | NOT Supported by cDNA | | | |
| 607 | GmaAffx.90331.1.S1_s_at | 0.243 | CF806056 | Disease & Defense | | | |
| 608 | GmaAffx.30421.1.S1_at | 0.243 | BU082092 | Transcription | Transcription | Zinc Finger | FOG: Predicted E3 ubiquitin ligase |
| | | | | | Factor | | |
| 609 | GmaAffx.22552.1.S1_at | 0.243 | BU549952 | Transporter | | | H+/oligopeptide symporter |
| 610 | GmaAffx.93301.2.S1_s_at | 0.242 | CF808497 | Secondary Metabolism | | | Flavonol reductase/cinnamoyl-CoA |
| | | | | | | | reductase |
| 611 | Gma.3531.1.S1_s_at | 0.241 | BI498803 | Unclassified - Hypothetical Protein | | | FOG: PPR repeat |
| | | | | Supported by cDNA | | | |
| 612 | Gma.4665.1.A1_at | 0.241 | AW309815 | No Homology to Known Proteins | | | |
| 613 | GmaAffx.24368.1.A1_at | 0.241 | BU545331 | No Homology to Known Proteins | | | |
| 614 | Gma.9086.1.S1_at | 0.24 | AW184932 | Cell Growth & Division | | | |
| 615 | Gma.6280.2.S1_at | 0.239 | AI938985 | No Homology to Known Proteins | | | |
| 616 | GmaAffx.66451.1.S1_at | 0.238 | AW309198 | Secondary Metabolism | | | |
| 617 | Gma.418.1.A1_at | 0.237 | CA784607 | Unclassified - Hypothetical Protein | | | Glycosyl transferase, family 8— |
| | | | | Supported by cDNA | | | glycogenin |
| 618 | Gma.13910.1.S1_at | 0.237 | CD409745 | Unclassified - Protein with | | | |
| | | | | Unknown Function | | | |
| 619 | Gma.17890.1.S1_at | 0.236 | AW432965 | No Homology to Known Proteins | | | |
| 620 | GmaAffx.89508.1.A1_s_at | 0.235 | CK606172 | Secondary Metabolism | | | Phenylalanine and histidine ammonia- |
| | | | | | | | lyase |
| 621 | Gma.10804.2.A1_s_at | 0.234 | CF921045 | Protein Destination & Storage | | | Molecular chaperone (DnaJ superfamily) |
| 622 | GmaAffx.52337.1.A1_at | 0.234 | CD413673 | No Homology to Known Proteins | | | |
| 623 | Gma.8988.1.S1_at | 0.234 | BU965547 | No Homology to Known Proteins | | | |
| 624 | Gma.7788.1.A1_at | 0.232 | BU549148 | Unclassified - Hypothetical Protein | | | Aspartyl protease |
| | | | | Supported by cDNA | | | |
| 625 | GmaAffx.92230.1.A1_s_at | 0.23 | CF807955 | Disease & Defense | | | |
| 626 | Gma.13856.1.A1_at | 0.23 | CD408222 | No Homology to Known Proteins | | | |
| 627 | GmaAffx.1242.1.A1_at | 0.229 | BU549839 | Metabolism | | | |
| 628 | Gma.15184.1.S1_at | 0.229 | BM522992 | Transcription | Transcription | Homeodomain | Transcription factor HEX, contains |
| | | | | | Factor | | HOX and HALZ domains |
| 629 | GmaAffx.73554.1.S1_at | 0.228 | BM139469 | Unclassified - Hypothetical Protein | | | |
| | | | | NOT Supported by cDNA | | | |
| 630 | Gma.6599.1.S1_at | 0.228 | BQ629084 | Cell Structure | | | |
| 631 | GmaAffx.83818.1.S1_at | 0.227 | BU762716 | No Homology to Known Proteins | | | |
| 632 | GmaAffx.54085.2.S1_at | 0.226 | BQ629483 | No Homology to Known Proteins | | | |
| 633 | GmaAffx.88017.1.A1_at | 0.226 | BQ452554 | No Homology to Known Proteins | | | |
| 634 | Gma.16528.1.S1_at | 0.225 | CD398706 | Unclassified - Hypothetical Protein | | | |
| | | | | Supported by cDNA | | | |
| 635 | Gma.8441.1.S1_at | 0.223 | AY113187.2 | Secondary Metabolism | | | Multicopper oxidases |
| 636 | GmaAffx.19467.1.S1_at | 0.22 | BQ453237 | Signal Transduction | Protein Kinase | | Serine/threonine protein kinase |
| 637 | Gma.4981.1.A1_at | 0.22 | BI970256 | No Homology to Known Proteins | | | |
| 638 | GmaAffx.81585.1.S1_at | 0.22 | BM187887 | Unclassified - Hypothetical Protein | | | Copper chaperone |
| | | | | NOT Supported by cDNA | | | |
| 639 | Gma.6642.1.A1_at | 0.219 | BE610052 | Transcription | Transcription | Basic Leucine | FOG: Armadillo/beta-catenin-like |
| | | | | | Factor | Zipper (bZIP) | repeats |
| 640 | GmaAffx.73943.1.S1_at | 0.218 | BM891115 | Unclassified - Hypothetical Protein | | | |
| | | | | Supported by cDNA | | | |
| 641 | GmaAffx.41230.1.A1_at | 0.218 | BI471609 | Unclassified - Hypothetical Protein | | | |
| | | | | NOT Supported by cDNA | | | |
| 642 | GmaAffx.92774.1.S1_at | 0.217 | CF808124 | Cell Structure | | | |
| 643 | Gma.3969.1.S1_s_at | 0.217 | AW349494 | Energy | | | |
| 644 | GmaAffx.21978.1.A1_at | 0.217 | CA803009 | No Homology to Known Proteins | | | |
| 645 | GmaAffx.34453.1.S1_at | 0.215 | BU544114 | Unclassified - Hypothetical Protein | | | |
| | | | | Supported by cDNA | | | |
| 646 | Gma.13218.1.A1_s_at | 0.213 | CD391892 | No Homology to Known Proteins | | | |
| 647 | GmaAffx.61130.1.S1_at | 0.213 | CA799668 | Transporter | | | H+/oligopeptide symporter |
| 648 | GmaAffx.41078.1.S1_at | 0.213 | BI317149 | Metabolism | | | Alcohol dehydrogenase, class III |
| 649 | GmaAffx.49777.1.S1_at | 0.212 | BG047001 | No Homology to Known Proteins | | | |
| 650 | Gma.7254.1.S1_at | 0.212 | BU546166 | Unclassified - Hypothetical Protein | | | |
| | | | | Supported by cDNA | | | |
| 651 | Gma.2376.1.S1_at | 0.209 | CA852095 | Transcription | Transcription | Homeodomain | Transcription factor MEIS1 and related |
| | | | | | Factor | | HOX domain proteins |
| 652 | Gma.3332.1.S1_at | 0.209 | BQ298780 | Protein Destination & Storage | | | |
| 653 | GmaAffx.88675.1.A1_at | 0.208 | CD417704 | Unclassified - Hypothetical Protein | | | |
| | | | | NOT Supported by cDNA | | | |
| 654 | Gma.18006.1.S1_at | 0.207 | CF922923 | Transcription | Transcription | Homeodomain | Transcription factor MEIS1 and related |
| | | | | | Factor | | HOX domain proteins |
| 655 | Gma.57.1.S1_at | 0.206 | AF532624.1 | Unclassified - Hypothetical Protein NOT | | | |
| | | | | Supported by cDNA | | | |
| 656 | Gma.5711.1.S1_at | 0.206 | BI784811 | Unclassified - Hypothetical Protein Supported | | | |
| | | | | by cDNA | | | |
| 657 | GmaAffx.56715.1.S1_at | 0.206 | BG041616 | Disease & Defense | | | |
| 658 | GmaAffx.42587.1.A1_at | 0.206 | BU544395 | No Homology to Known Proteins | | | |
| 659 | GmaAffx.8999.2.A1_s_at | 0.205 | BI700574 | No Homology to Known Proteins | | | |
| 660 | Gma.15907.2.S1_at | 0.205 | BE329550 | Signal Transduction | Unclassified | | FOG: Leucine rich repeat |
| 661 | GmaAffx.35656.1.A1_at | 0.204 | BE822348 | No Homology to Known Proteins | | | |
| 662 | GmaAffx.62509.1.S1_s_at | 0.204 | BE022332 | Transcription | Transcription | MYB | Transcription factor, Myb superfamily |
| | | | | | Factor | | |
| 663 | Gma.4746.1.A1_s_at | 0.203 | CD404836 | No Homology to Known Proteins | | | |
| 664 | Gma.149.1.S1_at | 0.203 | U43839.1 | Metabolism | | | Choline kinase |
| 665 | GmaAffx.64037.1.S1_at | 0.202 | BI321162 | Metabolism | Cytochrome | CYP82F | Cytochrome P450 CYP2 subfamily |
| | | | | | P450 | | |
| 666 | Gma.1929.1.S1_at | 0.201 | BU083650 | Transcription | Transcription | Homeodomain | Transcription factor HEX, contains |
| | | | | | Factor | | HOX and HALZ domains |
| 667 | Gma.2686.1.S1_s_at | 0.201 | BI970455 | Secondary Metabolism | | | |
| 668 | Gma.3987.2.S1_a_at | 0.2 | BI971594 | No Homology to Known Proteins | | | |
| 669 | GmaAffx.91641.1.S1_s_at | 0.2 | CF807366 | Disease & Defense | | | Acyl-CoA synthetase |
| 670 | Gma.3519.1.S1_at | 0.2 | CD403581 | Unclassified - Hypothetical Protein | | | |
| | | | | Supported by cDNA | | | |
| 671 | Gma.11522.2.S1_at | 0.199 | BG650872 | Unclassified - Hypothetical Protein | | | |
| | | | | Supported by cDNA | | | |
| 672 | GmaAffx.92649.1.S1_at | 0.198 | CF808374 | Metabolism | | | |
| 673 | GmaAffx.32700.1.S1_at | 0.196 | BM522940 | No Homology to Known Proteins | | | |
| 674 | Gma.17662.2.S1_s_at | 0.195 | BQ630190 | Unclassified - Hypothetical Protein | | | |
| | | | | NOT Supported by cDNA | | | |
| 675 | Gma.8966.1.S1_at | 0.19 | BU765374 | Protein Destination & Storage | | | Predicted lipase |
| 676 | Gma.6498.1.A1_at | 0.189 | BE824396 | Unclassified - Hypothetical Protein | | | |
| | | | | Supported by cDNA | | | |
| 677 | GmaAffx.6635.1.S1_at | 0.189 | CD412718 | Disease & Defense | | | |
| 678 | GmaAffx.74323.1.S1_at | 0.188 | BI426587 | Unclassified - Hypothetical Protein | | | Endoplasmic reticulum protein EP58, |
| | | | | NOT Supported by cDNA | | | contains filamin rod domain and KDEL |
| | | | | | | | motif |
| 679 | Gma.15625.1.S1_x_at | 0.188 | U77678.1 | Metabolism | Amino Acid | | Asparagine synthase (glutamine- |
| | | | | | Biosynthesis/Metabolism | | hydrolyzing) |
| 680 | GmaAffx.56301.1.S1_at | 0.188 | BQ610805 | No Homology to Known Proteins | | | |
| 681 | GmaAffx.92693.1.S1_at | 0.186 | CF808418 | No Homology to Known Proteins | | | |
| 682 | GmaAffx.27496.2.S1_at | 0.185 | BM523609 | Metabolism | | | |
| 683 | Gma.10312.1.S1_at | 0.184 | AW706614 | Unclassified - Hypothetical Protein | | | |
| | | | | NOT Supported by cDNA | | | |
| 684 | GmaAffx.81750.2.S1_at | 0.183 | BM568090 | Transcription | Transcription | Homeodomain | Transcription factor MEIS1 and related |
| | | | | | Factor | | HOX domain proteins |
| 685 | GmaAffx.43809.1.S1_at | 0.183 | AW396565 | No Homology to Known Proteins | | | |
| 686 | GmaAffx.68724.1.A1_at | 0.183 | BI970438 | No Homology to Known Proteins | | | |
| 687 | Gma.3951.1.S1_at | 0.182 | BE659337 | Unclassified - Hypothetical Protein | | | |
| | | | | NOT Supported by cDNA | | | |
| 688 | GmaAffx.14279.1.S1_at | 0.18 | BM887020 | Metabolism | | | |
| 689 | Gma.6481.1.S1_at | 0.178 | CD416703 | Unclassified - Hypothetical Protein | | | |
| | | | | Supported by cDNA | | | |
| 690 | GmaAffx.70008.1.S1_at | 0.178 | BU762337 | Transcription | Transcription | MYB | Transcription factor, Myb superfamily |
| | | | | | Factor | | |
| 691 | GmaAffx.21924.1.S1_at | 0.177 | BQ611867 | Disease & Defense | | | |
| 692 | Gma.9957.1.S1_at | 0.177 | BE820632 | Metabolism | | | UDP-glucuronosyl and UDP-glucosyl |
| | | | | | | | transferase |
| 693 | GmaAffx.40453.1.S1_at | 0.176 | BE658683 | No Homology to Known Proteins | | | |
| 694 | GmaAffx.71842.1.S1_at | 0.176 | BE555317 | Metabolism | | | Possible pfkB family carbohydrate |
| | | | | | | | kinase |
| 695 | GmaAffx.46837.1.S1_at | 0.175 | BU550919 | Secondary Metabolism | | | Multicopper oxidases |
| 696 | Gma.8490.1.S1_at | 0.175 | AB030492.1 | Metabolism | | | Protein involved in thiamine |
| | | | | | | | biosynthesis and DNA damage tolerance |
| 697 | GmaAffx.79390.1.S1_at | 0.174 | BU550458 | Transcription | Transcription | Basic Leucine | FOG: Armadillo/beta-catenin-like |
| | | | | | Factor | Zipper (bZIP) | repeats |
| 698 | Gma.1137.2.S1_x_at | 0.173 | CA852403 | Metabolism | | | |
| 699 | Gma.6081.1.S1_at | 0.173 | BI967474 | Disease & Defense | | | Predicted chitinase |
| 700 | Gma.3522.1.S1_at | 0.173 | BM140209 | Unclassified - Hypothetical Protein | | | |
| | | | | Supported by cDNA | | | |
| 701 | Gma.2801.1.S1_at | 0.173 | BI785739 | Cell Structure | | | Beta-glucosidase, lactase |
| | | | | | | | phlorizinhydrolase, and related proteins |
| 702 | Gma.8370.1.S1_at | 0.172 | BQ454087 | Signal Transduction | Protein | | Serine/threonine protein phosphatase |
| | | | | | Phosphatase | | |
| 703 | GmaAffx.89259.1.S1_s_at | 0.172 | CK605646 | Metabolism | | | |
| 704 | GmaAffx.93436.1.A1_s_at | 0.17 | CF809161 | Transcription | Transcription | AP2/EREBP | |
| | | | | | Factor | | |
| 705 | Gma.8704.1.S1_at | 0.17 | AW311022 | Unclassified - Hypothetical Protein | | | FOG: TPR repeat |
| | | | | NOT Supported by cDNA | | | |
| 705 | Gma.8704.1.S1_at | 0.17 | AW311022 | Unclassified - Hypothetical Protein | | | Thioredoxin |
| | | | | NOT Supported by cDNA | | | |
| 706 | GmaAffx.90677.1.S1_at | 0.17 | CF806402 | Unclassified - Hypothetical Protein | | | DNA helicase PIF1/RRM3 |
| | | | | NOT Supported by cDNA | | | |
| 707 | GmaAffx.47230.1.S1_at | 0.169 | CA820051 | Transporter | | | Amino acid transporters |
| 708 | Gma.16913.1.S1_s_at | 0.169 | BI426712 | Unclassified - Protein with | | | |
| | | | | Unknown Function | | | |
| 709 | Gma.4120.2.S1_at | 0.167 | BI700246 | Unclassified - Protein with | | | |
| | | | | Unknown Function | | | |
| 710 | Gma.3987.1.S1_at | 0.165 | BQ453408 | Unclassified - Hypothetical Protein | | | Sodium sulfate symporter and related |
| | | | | NOT Supported by cDNA | | | arsenite permeases |
| 711 | GmaAffx.62509.2.S1_at | 0.164 | BE021475 | Transcription | Transcription Factor | MYB | Transcription factor, Myb superfamily |
| 712 | GmaAffx.41917.1.S1_at | 0.164 | BE658926 | Disease & Defense | | | |
| 713 | Gma.4095.1.S1_at | 0.164 | AW350037 | Unclassified - Hypothetical Protein | | | |
| | | | | Supported by cDNA | | | |
| 714 | Gma.9036.1.S1_at | 0.163 | AW309501 | No Homology to Known Proteins | | | |
| 715 | GmaAffx.55959.1.S1_at | 0.163 | BQ629429 | Unclassified - Hypothetical Protein | | | |
| | | | | NOT Supported by cDNA | | | |
| 716 | Gma.3575.1.S1_at | 0.162 | BQ611599 | Cell Structure | | | |
| 717 | Gma.5961.1.S1_at | 0.162 | AB052786.1 | Transporter | | | H+/oligopeptide symporter |
| 718 | GmaAffx.39765.1.S1_s_at | 0.161 | BQ785586 | No Homology to Known Proteins | | | |
| 719 | Gma.4479.1.S1_at | 0.161 | CA936529 | Unclassified - Hypothetical Protein | | | |
| | | | | Supported by cDNA | | | |
| 720 | GmaAffx.89772.14.A1_s_at | 0.161 | CK606614 | Disease & Defense | | | |
| 721 | GmaAffx.11815.1.S1_at | 0.16 | AW311637 | Metabolism | | | |
| 722 | GmaAffx.77758.1.S1_x_at | 0.158 | BI944003 | No Homology to Known Proteins | | | |
| 723 | GmaAffx.15119.1.A1_at | 0.157 | BU544528 | Metabolism | | | Possible pfkB family carbohydrate |
| | | | | | | | kinase |
| 724 | Gma.15996.1.S1_at | 0.156 | BQ454193 | Unclassified - Hypothetical Protein | | | |
| | | | | Supported by cDNA | | | |
| 725 | GmaAffx.90812.1.S1_s_at | 0.155 | CF806537 | Disease & Defense | | | |
| 726 | Gma.18006.1.A1_at | 0.155 | AI496486 | Transcription | Transcription | Homeodomain | Transcription factor MEIS1 and related |
| | | | | | Factor | | HOX domain proteins |
| 727 | Gma.5863.2.S1_s_at | 0.155 | AW350762 | Secondary Metabolism | | | |
| 728 | Gma.17739.1.S1_at | 0.155 | AW310535 | Unclassified - Hypothetical Protein | | | |
| | | | | Supported by cDNA | | | |
| 729 | GmaAffx.81584.1.S1_at | 0.154 | BI426969 | Disease & Defense | | | |
| 730 | GmaAffx.5531.1.S1_at | 0.153 | BQ740711 | Cell Structure | | | |
| 731 | Gma.10621.1.S1_at | 0.153 | AY004243.1 | Unclassified - Hypothetical Protein | | | |
| | | | | Supported by cDNA | | | |
| 732 | Gma.14606.1.A1_at | 0.153 | BE821928 | Transporter | | | |
| 733 | Gma.11028.1.S1_s_at | 0.152 | BE823084 | Metabolism | | | Glyoxylate/hydroxypyruvate reductase |
| | | | | | | | (D-isomer-specific 2-hydroxy acid |
| | | | | | | | dehydrogenase superfamily) |
| 734 | Gma.8489.1.S1_s_at | 0.152 | AB030493.1 | Metabolism | | | Protein involved in thiamine |
| | | | | | | | biosynthesis and DNA damage tolerance |
| 735 | Gma.4150.2.S1_s_at | 0.149 | AW350622 | Unclassified - Hypothetical Protein | | | |
| | | | | Supported by cDNA | | | |
| 736 | GmaAffx.91555.1.A1_s_at | 0.148 | CF807280 | Metabolism | | | |
| 737 | Gma.6999.3.S1_s_at | 0.147 | CF921432 | Disease & Defense | | | |
| 738 | Gma.3604.1.S1_at | 0.147 | AW349604 | Cell Structure | | | O-methyltransferase |
| 739 | GmaAffx.91205.1.S1_at | 0.144 | CF807869 | Metabolism | | | Glyoxylate/hydroxypyruvate reductase |
| | | | | | | | (D-isomer-specific 2-hydroxy acid |
| | | | | | | | dehydrogenase superfamily) |
| 740 | GmaAffx.52963.1.S1_at | 0.144 | AW310673 | Unclassified - Protein with | | | |
| | | | | Unknown Function | | | |
| 741 | GmaAffx.59470.1.S1_at | 0.143 | BI469872 | Secondary Metabolism | | | Phenylalanine and histidine ammonia- |
| | | | | | | | lyase |
| 742 | GmaAffx.24992.1.S1_at | 0.143 | BU083490 | Protein Destination & Storage | | | Aspartyl protease |
| 743 | GmaAffx.72700.1.S1_at | 0.143 | AW349825 | Unclassified - Hypothetical Protein | | | |
| | | | | NOT Supported by cDNA | | | |
| 744 | Gma.6643.1.A1_at | 0.143 | BE823205 | Secondary Metabolism | | | Sulfotransferase |
| 745 | GmaAffx.14279.2.A1_at | 0.142 | BM095037 | Metabolism | | | |
| 746 | Gma.3799.2.S1_s_at | 0.142 | AW348702 | Unclassified - Protein with | | | |
| | | | | Unknown Function | | | |
| 747 | Gma.2842.1.S1_at | 0.141 | BG511652 | Unclassified - Protein with | | | Predicted Yippee-type zinc-binding |
| | | | | Unknown Function | | | protein |
| 748 | GmaAffx.87433.1.S1_at | 0.14 | BI701509 | Unclassified - Hypothetical Protein | | | |
| | | | | Supported by cDNA | | | |
| 749 | Gma.6278.1.S1_at | 0.137 | BQ612647 | Unclassified - Hypothetical Protein | | | |
| | | | | Supported by cDNA | | | |
| 750 | Gma.6643.2.S1_at | 0.137 | BE023121 | Secondary Metabolism | | | Sulfotransferase |
| 751 | Gma.5867.1.A1_s_at | 0.136 | BI316163 | Metabolism | | | |
| 752 | GmaAffx.81750.1.S1_at | 0.135 | AW306336 | No Homology to Known Proteins | | | |
| 753 | Gma.5863.1.S1_at | 0.134 | AJ003245.1 | Secondary Metabolism | | | |
| 754 | Gma.10969.1.S1_a_at | 0.133 | U36191.1 | Metabolism | | | |
| 755 | GmaAffx.3711.1.A1_at | 0.133 | BE822116 | Unclassified - Hypothetical Protein | | | |
| | | | | NOT Supported by cDNA | | | |
| 756 | GmaAffx.61593.1.S1_at | 0.133 | BU765478 | Unclassified - Hypothetical Protein | | | Sodium sulfate symporter and related |
| | | | | NOT Supported by cDNA | | | arsenite permeases |
| 757 | Gma.17446.1.A1_at | 0.132 | AI461103 | Unclassified - Protein with | | | |
| | | | | Unknown Function | | | |
| 758 | GmaAffx.17961.1.A1_at | 0.132 | BE657601 | Protein Destination & Storage | | | Molecular chaperones HSP70/HSC70, |
| | | | | | | | HSP70 superfamily |
| 759 | Gma.3776.1.S1_at | 0.131 | AW780736 | Metabolism | | | Glutaredoxin and related proteins |
| 760 | Gma.14471.1.S1_s_at | 0.131 | CF921241 | No Homology to Known Proteins | | | |
| 761 | GmaAffx.90677.1.S1_s_at | 0.128 | CF806402 | Unclassified - Hypothetical Protein | | | DNA helicase PIF1/RRM3 |
| | | | | NOT Supported by cDNA | | | |
| 762 | Gma.10969.3.S1_x_at | 0.128 | CD396552 | Metabolism | | | |
| 763 | Gma.4928.1.S1_a_at | 0.127 | AF002259.1 | Disease & Defense | | | Acyl-CoA synthetase |
| 764 | GmaAffx.93575.1.S1_s_at | 0.124 | CF808812 | No Homology to Known Proteins | | | |
| 765 | GmaAffx.82218.1.S1_at | 0.124 | BI702052 | Secondary Metabolism | | | Iron/ascorbate family oxidoreductases |
| 766 | Gma.4266.1.S1_at | 0.121 | BI972260 | No Homology to Known Proteins | | | |
| 767 | Gma.6999.1.S1_s_at | 0.119 | AF529303.1 | Disease & Defense | | | |
| 768 | GmaAffx.1301.78.S1_s_at | 0.119 | AW394497 | No Homology to Known Proteins | | | |
| 769 | Gma.3914.1.S1_at | 0.118 | BI702206 | Unclassified - Hypothetical Protein | | | |
| | | | | Supported by cDNA | | | |
| 770 | Gma.17877.1.S1_at | 0.118 | BQ612471 | Unclassified - Protein with | | | |
| | | | | Unknown Function | | | |
| 771 | GmaAffx.82000.1.S1_at | 0.115 | BI094895 | Unclassified - Hypothetical Protein | | | |
| | | | | Supported by cDNA | | | |
| 772 | Gma.1269.1.S1_at | 0.115 | AW309671 | Metabolism | | | Flavonol reductase/cinnamoyl-CoA |
| | | | | | | | reductase |
| 773 | Gma.14338.1.A1_at | 0.114 | CF923111 | Protein Destination & Storage | Storage Protein | | |
| 774 | Gma.6999.2.S1_s_at | 0.113 | X60043.1 | Disease & Defense | | | |
| 775 | Gma.9638.1.A1_at | 0.113 | CA936403 | Disease & Defense | | | |
| 776 | GmaAffx.34975.1.S1_at | 0.112 | BU547817 | Unclassified - Hypothetical Protein | | | |
| | | | | Supported by cDNA | | | |
| 777 | GmaAffx.478.1.S1_at | 0.112 | BE822248 | Transcription | Transcription | Homeodomain | Transcription factor MEIS1 and related |
| | | | | | Factor | | HOX domain proteins |
| 778 | Gma.1661.1.S1_at | 0.111 | BE821382 | Metabolism | | | |
| 779 | Gma.2133.1.S1_at | 0.11 | AW310071 | Unclassified - Protein with | | | |
| | | | | Unknown Function | | | |
| 780 | GmaAffx.19541.1.S1_s_at | 0.109 | BQ612990 | No Homology to Known Proteins | | | |
| 781 | GmaAffx.92927.1.S1_at | 0.109 | CF805992 | No Homology to Known Proteins | | | |
| 782 | Gma.9740.1.S1_at | 0.108 | BQ081058 | Unclassified - Hypothetical Protein | | | |
| | | | | Supported by cDNA | | | |
| 783 | Gma.15036.2.A1_at | 0.107 | CD411972 | Unclassified - Protein with | | | Predicted membrane protein |
| | | | | Unknown Function | | | |
| 784 | Gma.4120.1.A1_at | 0.106 | AW310714 | Unclassified - Protein with | | | |
| | | | | Unknown Function | | | |
| 785 | Gma.10227.1.A1_at | 0.106 | BE020065 | Metabolism | | | Flavin-containing monooxygenase |
| 786 | Gma.15568.1.S1_at | 0.103 | AI442650 | Disease & Defense | | | |
| 787 | GmaAffx.30665.1.A1_at | 0.103 | BQ453743 | Signal Transduction | Protein Kinase | | Serine/threonine protein kinase |
| 788 | GmaAffx.87670.1.S1_at | 0.103 | BQ452640 | Unclassified - Hypothetical Protein | | | |
| | | | | NOT Supported by cDNA | | | |
| 789 | GmaAffx.92479.1.S1_s_at | 0.101 | CF808204 | No Homology to Known Proteins | | | |
| 790 | GmaAffx.34449.1.S1_at | 0.101 | AW164447 | No Homology to Known Proteins | | | |
| 791 | GmaAffx.11897.1.S1_at | 0.1 | BE819868 | Transporter | | | Permease of the major facilitator |
| | | | | | | | superfamily |
| 792 | GmaAffx.83880.1.A1_at | 0.0946 | BE822640 | Disease & Defense | | | |
| 793 | Gma.1445.1.S1_at | 0.0927 | BQ454241 | Metabolism | | | Flavonol reductase/cinnamoyl-CoA |
| | | | | | | | reductase |
| 794 | GmaAffx.26516.1.S1_at | 0.0916 | AW433266 | Cell Structure | | | |
| 795 | Gma.5622.1.S1_at | 0.0895 | BG363493 | No Homology to Known Proteins | | | |
| 796 | GmaAffx.51464.1.S1_at | 0.089 | BQ628397 | No Homology to Known Proteins | | | |
| 797 | GmaAffx.51444.1.S1_at | 0.0883 | BM178639 | Secondary Metabolism | | | Multicopper oxidases |
| 798 | GmaAffx.74899.1.S1_at | 0.0855 | CA852377 | No Homology to Known Proteins | | | |
| 799 | Gma.15996.1.S1_x_at | 0.0827 | BQ454193 | Unclassified - Hypothetical Protein | | | |
| | | | | Supported by cDNA | | | |
| 800 | Gma.4610.1.S1_s_at | 0.073 | BM522856 | No Homology to Known Proteins | | | |
| 801 | Gma.79.1.S1_x_at | 0.0729 | S44202.1 | Unclassified - Protein with | | | |
| | | | | Unknown Function | | | |
| 802 | Gma.1243.1.S1_at | 0.0725 | AW311027 | Unclassified - Hypothetical Protein | | | |
| | | | | Supported by cDNA | | | |
| 803 | Gma.5947.1.S1_s_at | 0.0722 | BQ610509 | Disease & Defense | | | |
| 804 | Gma.9010.1.S1_at | 0.0719 | CF921194 | Disease & Defense | | | |
| 805 | Gma.10216.1.S1_at | 0.0693 | BI969369 | Metabolism | | | Hydroxyindole-O-methyltransferase and |
| | | | | | | | related SAM-dependent |
| | | | | | | | methyltransferases |
| 806 | Gma.11211.1.S1_s_at | 0.0639 | AI442578 | No Homology to Known Proteins | | | |
| 807 | Gma.17840.1.S1_at | 0.0614 | BE821995 | No Homology to Known Proteins | | | |
| 808 | Gma.5611.1.S1_at | 0.0612 | AW309826 | Metabolism | Cytochrome | CYP81D | Cytochrome P450 CYP2 subfamily |
| | | | | | P450 | | |
| 809 | Gma.79.1.S1_s_at | 0.0565 | S44202.1 | Unclassified - Protein with | | | |
| | | | | Unknown Function | | | |
| 810 | Gma.4401.2.S1_a_at | 0.0559 | BI970725 | Disease & Defense | | | |
| 811 | Gma.8425.1.S1_s_at | 0.0512 | D83968.1 | Metabolism | Cytochrome | CYP93D | Cytochrome P450 CYP2 subfamily |
| | | | | | P450 | | |
| 812 | Gma.4796.1.A1_at | 0.0486 | AW102323 | Unclassified - Hypothetical Protein | | | Uncharacterized conserved protein |
| | | | | NOT Supported by cDNA | | | |
| 813 | Gma.15307.1.S1_at | 0.0401 | U64866.1 | Disease & Defense | | | |
| 814 | GmaAffx.85382.1.S1_s_at | 0.0377 | BI945154 | No Homology to Known Proteins | | | |

TABLE 15

| | | | | Sub- | Gene | |
|---|---|---|---|---|---|---|
| ID | Gene Name | Soybean_GenBank_Acc | Functional Category | Family | Family | Additional Description |
| 1 | Gma.1248.1.S1_s_at | AJ518837.1 | Metabolism | | | Haloacid dehalogenase- |
| | | | | | | like hydrolase |
| 2 | GmaAffx.93529.1.S1_s_at | CF809254 | No Homology to | | | |
| | | | Known Proteins | | | |
| 3 | GmaAffx.9605.1.S1_at | BM091603 | No Homology to | | | |
| | | | Known Proteins | | | |
| 4 | Gma.3405.1.S1_s_at | AW350922 | Metabolism | | | Branched chain |
| | | | | | | aminotransferase BCAT1, |
| | | | | | | pyridoxal phosphate enzymes |
| | | | | | | type IV superfamily |
| 5 | Gma.7332.1.S1_at | B1945636 | Unclassified - | | | |
| | | | Hypothetical Protein | | | |
| | | | Supported by cDNA | | | |
| 6 | GmaAffx.3905.1.S1_s_at | CA818952 | Unclassified - | | | |
| | | | Protein with Unknown | | | |
| | | | Function | | | |
| 7 | GmaAffx.3905.3.S1_at | BG047142 | Unclassified - | | | |
| | | | Protein with Unknown | | | |
| | | | Function | | | |
| 8 | GmaAffx.90947.1.S1_at | CF806672 | Unclassified - | | | |
| | | | Protein with Unknown | | | |
| | | | Function | | | |
| 9 | Gma.2548.1.S1_at | BE821472 | Unclassified - | | | |
| | | | Hypothetical Protein | | | |
| | | | Supported by cDNA | | | |
| 10 | GmaAffx.10773.1.S1_at | BU926045 | Unclassified - | | | |
| | | | Hypothetical Protein | | | |
| | | | Supported by cDNA | | | |
| 11 | Gma.4051.1.S1_at | L22162.1 | Cell Structure | | | |
| 12 | GmaAffx.9400.1.S1_at | BI893703 | Signal Transduction | Protein | | Serine/threonine |
| | | | | Kinase | | protein kinase |
| 13 | Gma.9141.1.A1_at | AW310721 | Secondary Metabolism | | | |
| 14 | GmaAffx.61907.1.S1_at | CA819279 | No Homology to | | | |
| | | | Known Proteins | | | |
| 15 | GmaAffx.83598.1.S1_at | BU964918 | Transcription | Transcription | AP2/EREBP | |
| | | | | Factor | | |
| 16 | Gma.17887.1.S1_s_at | BG042115 | Unclassified - | | | |
| | | | Hypothetical Protein | | | |
| | | | Supported by cDNA | | | |
| 17 | GmaAffx.55404.1.S1_at | BM271530 | No Homology to | | | |
| | | | Known Proteins | | | |
| 18 | Gma.3104.1.A1_at | BG839320 | Signal Transduction | Protein | | Serine/threonine |
| | | | | Kinase | | protein kinase |
| 19 | GmaAffx.12370.1.S1_at | BE059411 | Cell Growth | | | Microtubule-associated |
| | | | & Division | | | protein essential for |
| | | | | | | anaphase spindle elongation |
| 20 | GmaAffx.85255.1.S1_at | BE059755 | No Homology to | | | |
| | | | Known Proteins | | | |
| 21 | GmaAffx.60984.1.S1_at | BU765206 | Transporter | | | |
| 22 | Gma.10573.3.S1_at | CA938507 | Transcription | Transcription | Zinc | GATA-4/5/6 |
| | | | | Factor | Finger | transcription factors |
| 23 | GmaAffx.93561.1.S1_at | CF807977 | Metabolism | | | Branched chain |
| | | | | | | aminotransferase BCAT1, |
| | | | | | | pyridoxal phosphate enzymes |
| | | | | | | type IV superfamily |
| 24 | GmaAffx.78599.1.A1_at | CA784082 | No Homology to | | | |
| | | | Known Proteins | | | |
| 25 | GmaAffx.77804.1.A1_at | B1968697 | No Homology to | | | |
| | | | Known Proteins | | | |
| 26 | Gma.6719.3.S1_s_at | AW164562 | Unclassified - | | | |
| | | | Hypothetical Protein | | | |
| | | | NOT Supported by | | | |
| | | | cDNA | | | |
| 27 | GmaAffx.74847.1.S1_at | BG507699 | Metabolism | | | |
| 28 | GmaAffx.68754.1.S1_at | BE474746 | Secondary | | | Hydroxyindole-O- |
| | | | Metabolism | | | methyltransferase and |
| | | | | | | related SAM-dependent |
| | | | | | | methyltransferases |
| 29 | Gma.4414.1.A1_at | BQ081678 | No Homology to | | | |
| | | | Known Proteins | | | |
| 30 | GmaAffx.74674.1.S1_s_at | BU965464 | Transporter | Auxin | | |
| | | | | Transport | | |
| 31 | GmaAffx.57878.2.S1_at | BE610836 | Cell Structure | | | |
| 32 | GmaAffx.92536.1.S1_s_at | CF808261 | Secondary | Ethylene | | Iron/ascorbate family |
| | | | Metabolism | Biosynthesis | | oxidoreductases |
| 33 | Gma.17638.2.S1_at | BI786260 | Transcription | Transcription | Unclas- | GATA-4/5/6 |
| | | | | Factor | sified | transcription factors |
| 34 | GmaAffx.13039.1.A1_at | BF219584 | Secondary | | | |
| | | | Metabolism | | | |
| 35 | GmaAffx.47451.1.S1_at | CA799081 | Unclassified - | | | |
| | | | Hypothetical Protein | | | |
| | | | NOT Supported by | | | |
| | | | cDNA | | | |
| 36 | Gma.17502.2.S1_x_at | BG046201 | Transcription | Transcription | Unclas- | Zuotin and related |
| | | | | Factor | sified | molecular chaperones |
| | | | | | | (DnaJ superfamily), contains |
| | | | | | | DNA-binding domains |
| 37 | GmaAffx.73059.1.S1_at | B1426008 | Unclassified - | | | Predicted |
| | | | Hypothetical Protein | | | dehydrogenase |
| | | | NOT Supported by | | | |
| | | | cDNA | | | |
| 38 | GmaAffx.18376.1.A1_at | BI321742 | Protein | | | Ubiquitin-protein |
| | | | Destination & | | | ligase |
| | | | Storage | | | |
| 39 | Gma.13800.1.A1_at | CD405906 | Unclassified - | | | |
| | | | Hypothetical Protein | | | |
| | | | NOT Supported by | | | |
| | | | cDNA | | | |
| 40 | GmaAffx.78561.1.S1_at | BQ452483 | Metabolism | | | UDP-glucuronosyl |
| | | | | | | and UDP-glucosyl |
| | | | | | | transferase |
| 41 | GmaAffx.35908.1.S1_at | BF598958 | No Homology to | | | |
| | | | Known Proteins | | | |
| 42 | Gma.2780.1.A1_at | AI988066 | Unclassified - | | | UDP-galactose |
| | | | Hypothetical Protein | | | transporter |
| | | | NOT Supported by | | | related protein |
| | | | cDNA | | | |
| 43 | GmaAffx.91143.1.S1_at | CF806868 | Signal Transduction | Unclassified | | |
| 44 | Gma.4400.1.S1_at | BI317776 | Transcription | Transcription | WRKY | |
| | | | | Factor | | |
| 45 | GmaAffx.73452.2.S1_at | BM085321 | Signal Transduction | Phytochrome | | eIF-2alpha kinase |
| | | | | | | PEK/EIF2AK3 |
| 46 | GmaAffx.60057.1.S1_at | BM308158 | No Homology to | | | |
| | | | Known Proteins | | | |
| 47 | Gma.2776.2.S1_s_at | BU577475 | Secondary | Ethylene | | Iron/ascorbate family |
| | | | Metabolism | Biosynthesis | | oxidoreductases |
| 48 | GmaAffx.91807.1.S1_at | CF807532 | No Homology to | | | |
| | | | Known Proteins | | | |
| 49 | GmaAffx.85999.1.S1_at | BF426048 | Unclassified - | | | |
| | | | Hypothetical Protein | | | |
| | | | Supported by | | | |
| | | | cDNA | | | |
| 50 | Gma.5159.1.A1_at | BI971147 | No Homology to | | | |
| | | | Known Proteins | | | |
| 51 | GmaAffx.9280.1.S1_at | BI498912 | Signal Transduction | Calcium | Calcium | |
| | | | | Binding | Dependent | |
| | | | | | Protein | |
| | | | | | Kinase | |
| 52 | GmaAffx.4036.1.S1_at | BM527313 | Unclassified - | | | |
| | | | Hypothetical Protein | | | |
| | | | Supported by | | | |
| | | | cDNA | | | |
| 53 | Gma.15468.1.S1_at | BE800435 | Disease & Defense | | | |
| 54 | GmaAffx.85287.2.S1_at | BI472203 | Unclassified - | | | |
| | | | Hypothetical Protein | | | |
| | | | Supported by | | | |
| | | | cDNA | | | |
| 55 | GmaAffx.83016.1.S1_at | BQ080317 | Transcription | Transcription | Zinc | |
| | | | | Factor | Finger | |
| 56 | GmaAffx.29616.1.A1_at | CD404723 | No Homology to | | | |
| | | | Known Proteins | | | |
| 57 | PsAffx.C107000004_at | | | | | |
| 58 | GmaAffx.61777.1.S1_at | A1965744 | Signal | Protein | | |
| | | | Transduction | Kinase | | |
| 59 | GmaAffx.14153.2.S1_at | BE329834 | Unclassified - | | | |
| | | | Hypothetical Protein | | | |
| | | | Supported by | | | |
| | | | cDNA | | | |
| 60 | GmaAffx.29951.2.S1_at | AW100899 | Metabolism | | | Dehydrogenase kinase |
| 61 | GmaAffx.12142.1.S1_at | AW733336 | Metabolism | Cytochrome | CYP716A | Cytochrome P450 |
| | | | | P450 | | CYP4/CYP19/CYP26 |
| | | | | | | subfamilies |
| 62 | Gma.8262.1.A1_at | BU550168 | Signal | Protein | | Putative serine/threonine |
| | | | Transduction | Kinase | | protein kinase |
| 63 | Gma.1164.1.S1_at | BE822845 | Energy | | | Ferric reductase, |
| | | | | | | NADH/NADPH oxidase |
| | | | | | | and related proteins |
| 64 | Gma.17323.1.S1_at | AW567763 | No Homology to | | | |
| | | | Known Proteins | | | |
| 65 | Gma.10216.3.A1_x_at | B1969715 | Metabolism | | | Hydroxyindole-O- |
| | | | | | | methyltransferase and |
| | | | | | | related SAM-dependent |
| | | | | | | methyltransferases |
| 66 | GmaAffx.92791.1.S1_at | CF808516 | Metabolism | | | Reductases with broad |
| | | | | | | range of substrate |
| | | | | | | specificities |
| 67 | GmaAffx.60479.2.S1_at | BU765815 | Transcription | Transcription | Homeo- | Transcription factor |
| | | | | Factor | domain | ME1S1 and related |
| | | | | | | HOX domain proteins |
| 68 | PsAffx.psMA015xP13f_at | | | | | |
| 69 | GmaAffx.31640.1.S1_at | BG237979 | No Homology to | | | |
| | | | Known Proteins | | | |
| 70 | GmaAffx.26767.1.S1_at | BF010276 | Transcription | Transcription | MYB | Transcription factor, |
| | | | | Factor | | Myb superfamily |
| 71 | Gma.13452.1.A1_at | CD398445 | No Homology to | | | |
| | | | Known Proteins | | | |
| 72 | Gma.649.1.S1_at | AW568984 | No Homology to | | | |
| | | | Known Proteins | | | |
| 73 | GmaAffx.59252.1.A1_at | AI442669 | Protein | | | FOG: Predicted E3 |
| | | | Destination & | | | ubiquitin ligase |
| | | | Storage | | | |
| 74 | GmaAffx.63054.1.S1_at | BE806784 | Unclassified - | | | |
| | | | Hypothetical Protein | | | |
| | | | NOT Supported by | | | |
| | | | cDNA | | | |
| 75 | GmaAffx.14016.1.S1_at | BE824450 | No Homology to | | | |
| | | | Known Proteins | | | |
| 76 | Gma.3581.2.S1_a_at | CD398783 | Unclassified - | | | Ca2+-dependent lipid- |
| | | | Hypothetical Protein | | | binding protein CLB1/vesicle |
| | | | NOT Supported by | | | protein vp115/Granuphilin |
| | | | cDNA | | | A, contains C2 domain |
| 77 | Gma.17321.1.S1_at | AW508844 | Metabolism | | | Tyrosine aminotransferase |
| 78 | Gma.7301.1.S1_at | BM528250 | Disease & Defense | | | Gamma-glutamyltransferase |
| 79 | Gma.7608.1.S1_at | BU549027 | Unclassified - | | | RNA-binding protein |
| | | | Hypothetical Protein | | | Sam68 and related |
| | | | NOT Supported by | | | KH domain proteins |
| | | | cDNA | | | |
| 80 | GmaAffx.6571.1.S1_at | CD403804 | Metabolism | | | UDP-glucuronosyl |
| | | | | | | and UDP-glucosyl |
| | | | | | | transferase |
| 81 | GmaAffx.934.1.S1_at | BG046802 | No Homology to | | | |
| | | | Known Proteins | | | |
| 82 | Gma.5124.1.S1_at | BI971079 | Protein | | | |
| | | | Destination & | | | |
| | | | Storage | | | |
| 83 | Gma.12212.1.A1_s_at | CD396776 | No Homology to | | | |
| | | | Known Proteins | | | |
| 84 | GmaAffx.9400.2.S1_at | BI945484 | Signal | Protein | | Glycerophosphoryl diester |
| | | | Transduction | Kinase | | phosphodiesterase |
| 84 | GmaAffx.9400.2.S1_at | BI945484 | Signal | Protein | | Serine/threonine |
| | | | Transduction | Kinase | | protein kinase |
| 85 | GmaAffx.47228.1.S1_at | BU761053 | Transporter | | | |
| 86 | GmaAffx.71589.1.S1_at | BE020161 | Cell Growth | | | |
| | | | & Division | | | |
| 87 | GmaAffx.75219.1.S1_at | BE020773 | Unclassified - | | | Fasciclin and |
| | | | Hypothetical Protein | | | related adhesion |
| | | | NOT Supported by | | | glycoproteins |
| | | | cDNA | | | |
| 88 | GmaAffx.40086.1.S1_at | BE659815 | Unclassified - | | | |
| | | | Hypothetical Protein | | | |
| | | | Supported by cDNA | | | |
| 89 | Gma.3591.1.S1_x_at | BE440266 | Secondary | Ethylene | | Iron/ascorbate family |
| | | | Metabolism | Biosynthesis | | oxidoreductases |
| 90 | Gma.16526.1.S1_ at | CD407998 | Protein | | | Serine |
| | | | Destination & | | | carboxypeptidases |
| | | | Storage | | | (lysosomal cathepsin A) |
| 91 | GmaAffx.46684.1.S1_at | BQ081034 | Signal | Protein | | |
| | | | Transduction | Kinase | | |
| 92 | Gma.285.1.S1_at | BG726992 | Cell Growth | | | |
| | | | & Division | | | |
| 93 | GmaAffx.86136.1.S1_at | A1440673 | Unclassified - | | | |
| | | | Protein with | | | |
| | | | Unknown Function | | | |
| 94 | Gma.10026.1.S1_at | BU546301 | Unclassified - | | | |
| | | | Protein with | | | |
| | | | Unknown Function | | | |
| 95 | GmaAffx.86392.1.S1_at | AI736754 | Signal | Calcium- | | |
| | | | Transduction | Binding | | |
| 96 | Gma.1839.2.S1_a_at | AW507949 | Metabolism | | | |
| 97 | Gma.17759.1.S1_at | BU927041 | Transcription | Transcription | AUX/IAA | |
| | | | | Factor | | |
| 98 | GmaAffx.67113.1.S1_at | BE329541 | No Homology to | | | |
| | | | Known Proteins | | | |
| 99 | GmaAffx.84209.1.S1_at | BQ610588 | Signal | Histidine | | Sensory transduction |
| | | | Transduction | Kinase | | histidine kinase |
| 100 | Gma.10144.2.S1_at | BU080819 | Metabolism | | | |
| 101 | GmaAffx.9040.1.S1_at | AW101738 | Unclassified - | | | |
| | | | Hypothetical Protein | | | |
| | | | Supported by | | | |
| | | | cDNA | | | |
| 102 | GmaAffx.86514.1.S1_at | BG790017 | Transcription | Transcription | ARF | |
| | | | | Factor | | |
| 103 | GmaAffx.18880.1.S1_at | BM094235 | Signal | Protein Kinase | | Serine/threonine |
| | | | Transduction | | | protein kinase |
| 104 | GmaAffx.65140.1.S1_at | BI700031 | No Homology to | | | |
| | | | Known Proteins | | | |
| 105 | GmaAffx.55953.1.S1_at | BQ629142 | Unclassified - | | | FOG: Predicted E3 |
| | | | Hypothetical Protein | | | ubiquitin ligase |
| | | | NOT Supported by | | | |
| | | | cDNA | | | |
| 106 | Gma.5559.1.S1_s_at | BM121524 | Unclassified - | | | |
| | | | Hypothetical Protein | | | |
| | | | Supported by | | | |
| | | | cDNA | | | |
| 107 | GmaAffx.84206.1.S1_at | BU760571 | Transcription | Transcription | NAC | |
| | | | | Factor | Domain | |
| 108 | GmaAffx.47230.2.S1_at | BG652486 | Transporter | | | Amino acid transporters |
| 109 | GmaAffx.39703.1.S1_at | BE023656 | Protein | | | Serine |
| | | | Destination & | | | carboxypeptidases |
| | | | Storage | | | (lysosomal cathepsin A) |
| 110 | GmaAffx.20146.1.S1_at | BE824274 | Unclassified - | | | Ca2+-binding protein |
| | | | Protein with | | | (centrin/caltractin), |
| | | | Unknown Function | | | EF-Hand superfamily protein |
| 111 | GmaAffx.71016.1.S1_at | AW234455 | Unclassified - | | | Arylacetamide |
| | | | Hypothetical Protein | | | deacetylase |
| | | | Supported by | | | |
| | | | cDNA | | | |
| 112 | GmaAffx.91141.1.S1_at | CF806866 | Disease & Defense | | | |
| 113 | GmaAffx.93373.1.S1_s_at | CF809098 | Unclassified - | | | |
| | | | Hypothetical Protein | | | |
| | | | Supported by | | | |
| | | | cDNA | | | |
| 114 | GmaAffx.44396.1.S1_at | BE330360 | No Homology to | | | |
| | | | Known Proteins | | | |
| 115 | GmaAffx.80900.1.S1_at | BE347494 | Metabolism | | | Iron/ascorbate family |
| | | | | | | oxidoreductases |
| 116 | Gma.6727.1.S1_at | Y10493.1 | Metabolism | Cytochrome | CYP71B | Cytochrome P450 |
| | | | | P450 | | CYP2 subfamily |
| 117 | GmaAffx.99.4.S1_at | BQ742171 | Unclassified - | | | Microtubule-binding protein |
| | | | Protein with | | | (translationally controlled |
| | | | Unknown Function | | | tumor protein) |
| 118 | Gma.13736.1.A1_at | CD404408 | Secondary | | | Multicopper oxidases |
| | | | Metabolism | | | |
| 119 | GmaAffx.83377.2.S1_at | BM893101 | Transcription | Transcription | Basic | |
| | | | | Factor | Helix- | |
| | | | | | Loop- | |
| | | | | | Helix | |
| | | | | | (bHLH) | |
| 120 | GmaAffx.91960.1.S1_at | CF807685 | Metabolism | | | Branched chain alpha-keto |
| | | | | | | acid dehydrogenase complex, |
| | | | | | | alpha subunit |
| 121 | Gma.6442.2.S1_at | BM270161 | Metabolism | | | Lysophospholipase |
| 122 | Gma.15155.1.S1_a_at | BF423817 | Unclassified - | | | |
| | | | Hypothetical Protein | | | |
| | | | Supported by | | | |
| | | | cDNA | | | |
| 123 | Gma.10559.1.S1_s_at | BU544359 | No Homology to | | | |
| | | | Known Proteins | | | |
| 124 | GmaAffx.16986.2.S1_at | AW277368 | Metabolism | | | Hydroxyindole-O- |
| | | | | | | methyltransferase and |
| | | | | | | related SAM-dependent |
| | | | | | | methyltransferases |
| 125 | Gma.2414.1.S1_at | BU551192 | Protein | | | |
| | | | Destination & | | | |
| | | | Storage | | | |
| 126 | GmaAffx.51409.1.S1_at | BM309035 | Transporter | | | Synaptic vesicle |
| | | | | | | transporter SVOP and |
| | | | | | | related transporters (major |
| | | | | | | facilitator superfamily) |
| 127 | Gma.6679.1.A1_at | BU544081 | Transcription | Transcription | MYB | Transcription factor, |
| | | | | Factor | | Myb superfamily |
| 128 | Gma.4674.1.A1_at | AW309836 | Metabolism | | | Arylacetamide deacetylase |
| 129 | Gma.10559.1.S1_at | BU544359 | No Homology to | | | |
| | | | Known Proteins | | | |
| 130 | GmaAffx.45334.1.S1_at | BI969317 | Secondary | GA | | Iron/ascorbate family |
| | | | Metabolism | Biosynthesis | | oxidoreductases |
| 131 | GmaAffx.16986.1.A1_s_at | AW351116 | Metabolism | | | Hydroxyindole-O- |
| | | | | | | methyltransferase and |
| | | | | | | related SAM-dependent |
| | | | | | | methyltransferases |

TABLE 16

| | | | | Sub- | Gene | |
|---|---|---|---|---|---|---|
| ID | Gene Name | Soybean_ GenBank_Acc | Functional Category | Family | Family | Additional Description |
| 1 | GmaAffx.66589.1.S1_at | BE821501 | Transcription | Transcription | Polycomb | |
| | | | | Factor | Group | |
| 2 | Gma.10307.1.S1_at | CA936845 | Unclassified - | | | |
| | | | Hypothetical Protein | | | |
| | | | NOT Supported by | | | |
| | | | cDNA | | | |
| 3 | GmaAffx.13179.1.S1_at | CD402274 | Unclassified - | | | |
| | | | Hypothetical Protein | | | |
| | | | NOT Supported by | | | |
| | | | cDNA | | | |
| 4 | GmaAffx.30420.1.S1_at | AW704927 | Transporter | | | |
| 5 | Gma.14802.1.S1_at | AW201116 | Unclassified - | | | FOG: |
| | | | Protein with | | | Ankyrin repeat |
| | | | Unknown Function | | | |
| 6 | Gma.869.1.S1_at | BE822531 | Protein | | | Serine |
| | | | Destination | | | carboxypeptidases |
| | | | & Storage | | | (lysosomal |
| | | | | | | cathepsin A) |
| 7 | GmaAffx.56804.1.A1_at | CD415337 | Unclassified - | | | Uncharacterized |
| | | | Hypothetical Protein | | | conserved protein, |
| | | | NOT Supported by | | | contains TraB |
| | | | cDNA | | | domain |
| 8 | GmaAffx.83103.2.S1_at | BE806704 | Unclassified - | | | |
| | | | Hypothetical Protein | | | |
| | | | NOT Supported by | | | |
| | | | cDNA | | | |
| 9 | GmaAffx.87806.1.S1_at | BG882829 | Unclassified - | | | |
| | | | Hypothetical Protein | | | |
| | | | NOT Supported by | | | |
| | | | cDNA | | | |
| 10 | Gma.10787.2.S1_a_at | BE023083 | Transcription | Transcription | Squamosa | |
| | | | | Factor | | |
| 11 | GmaAffx.84721.1.S1_at | BQ297863 | Metabolism | | | Hydroxymethylglutaryl- |
| | | | | | | CoA lyase |
| 12 | Gma.12821.1.S1_s_at | CD391504 | No Homology to | | | |
| | | | Known Proteins | | | |
| 13 | GmaAffx.71386.1.S1_at | AW704747 | Protein | | | Molecular chaperone |
| | | | Destination | | | (DnaJ superfamily) |
| | | | & Storage | | | |
| 14 | Gma.8782.1.A1_at | AW309953 | Disease & Defense | | | Apoptotic ATPase |
| 15 | Gma.17191.1.S1_at | BG237724 | Metabolism | | | UDP-glucuronosyl |
| | | | | | | and UDP-glucosyl |
| | | | | | | transferase |
| 16 | GmaAffx.77527.1.S1_at | BF423510 | Unclassified - | | | GTPase Rab18, |
| | | | Hypothetical Protein | | | small G protein |
| | | | Supported by | | | superfamily |
| | | | cDNA | | | |
| 17 | GmaAffx.87922.1.S1_at | BU084547 | Energy | | | Pyrophosphate- |
| | | | | | | dependent |
| | | | | | | phosphofructo- |
| | | | | | | 1-kinase |
| 18 | GmaAffx.75708.1.S1_at | BF599014 | Unclassified - | | | |
| | | | Hypothetical Protein | | | |
| | | | Supported by | | | |
| | | | cDNA | | | |
| 19 | Gma.6025.1.S1_at | CD399734 | Unclassified - | | | |
| | | | Hypothetical Protein | | | |
| | | | NOT Supported by | | | |
| | | | cDNA | | | |
| 20 | GmaAffx.36972.2.S1_at | BI699120 | Unclassified - | | | |
| | | | Hypothetical Protein | | | |
| | | | NOT Supported by | | | |
| | | | cDNA | | | |
| 21 | Gma.6528.2.S1_at | CA784209 | Transporter | | | Calcium transporting |
| | | | | | | ATPase |
| 22 | Gma.7835.1.S1_at | CD410897 | Cell Growth | | | |
| | | | & Division | | | |
| 23 | GmaAffx.6565.1.A1_at | CD402930 | No Homology to | | | |
| | | | Known Proteins | | | |
| 24 | GmaAffx.71778.1.S1_at | BE346999 | Unclassified - | | | |
| | | | Hypothetical Protein | | | |
| | | | NOT Supported by | | | |
| | | | cDNA | | | |
| 25 | GmaAffx.53585.2.S1_at | BE556399 | Protein | | | Molecular chaperone |
| | | | Destination | | | (DnaJ superfamily) |
| | | | & Storage | | | |
| 26 | GmaAffx.37484.2.S1_at | BQ629925 | Unclassified - | | | Predicted membrane |
| | | | Protein with | | | protein, contains |
| | | | Unknown Function | | | DoH and Cytochrome |
| | | | | | | b-561/ferric reductase |
| | | | | | | transmembrane domains |
| 27 | Gma.14155.3.S1_at | AI437500 | Unclassified - | | | Clathrin assembly |
| | | | Hypothetical Protein | | | protein AP180 and |
| | | | Supported by | | | related proteins, |
| | | | cDNA | | | contain ENTH domain |
| 28 | Gma.1677.2.A1_at | AW348386 | Secondary | | | |
| | | | Metabolism | | | |
| 29 | GmaAffx.50632.1.A1_at | BI969115 | Metabolism | | | Malate dehydrogenase |
| 30 | Gma.6722.1.S1_at | BE822185 | Unclassified - | | | UDP-glucuronosyl |
| | | | Hypothetical Protein | | | and UDP-glucosyl |
| | | | NOT Supported by | | | transferase |
| | | | cDNA | | | |
| 31 | GmaAffx.11383.1.S1_at | BE822014 | Unclassified - | | | Kinesin light |
| | | | Hypothetical Protein | | | chain |
| | | | NOT Supported by | | | |
| | | | cDNA | | | |
| 32 | GmaAffx.77651.1.S1_at | BF010272 | Unclassified - | Hormone | | Zinc-binding |
| | | | Protein with | Induced | | oxidoreductase |
| | | | Unknown Function | | | |
| 33 | Gma.9717.1.S1_at | BG653084 | Unclassified - | | | Amino acid |
| | | | Hypothetical Protein | | | transporters |
| | | | NOT Supported by | | | |
| | | | cDNA | | | |
| 34 | GmaAffx.21553.1.S1_at | BE023327 | Protein | | | Gelatinase A and |
| | | | Destination | | | related matrix |
| | | | & Storage | | | metalloproteases |
| 35 | GmaAffx.90952.1.S1_at | CF806677 | Signal | Unclassified | | |
| | | | Transduction | | | |
| 36 | GmaAffx.84871.1.S1_at | BF599103 | Signal | Protein | | |
| | | | Transduction | Kinase | | |
| 37 | GmaAffx.56020.1.S1_at | BU080527 | Unclassified - | | | |
| | | | Hypothetical Protein | | | |
| | | | NOT Supported by | | | |
| | | | cDNA | | | |
| 38 | GmaAffx.765.2.S1_at | AW509310 | Transporter | | | Amino acid |
| | | | | | | transporters |
| 39 | GmaAffx.86283.1.S1_at | BI892496 | Unclassified - | | | Predicted |
| | | | Hypothetical Protein | | | pyroglutamyl |
| | | | Supported by | | | peptidase |
| | | | cDNA | | | |
| 40 | Gma.9436.1.S1_at | CA802734 | Unclassified - | | | |
| | | | Hypothetical Protein | | | |
| | | | NOT Supported by | | | |
| | | | cDNA | | | |
| 41 | Gma.16441.1.S1_a_at | BU548048 | Unclassified - | | | HGG |
| | | | Hypothetical Protein | | | motif-containing |
| | | | NOT Supported by | | | thioesterase |
| | | | cDNA | | | |
| 42 | Gma.1058.1.S1_at | BE820580 | Metabolism | | | |
| 43 | Gma.17305.1.S1_at | AW351319 | Unclassified - | | | |
| | | | Protein with | | | |
| | | | Unknown Function | | | |
| 44 | Gma.15729.1.A1_at | BI971055 | Cell Growth | | | |
| | | | & Division | | | |
| 45 | GmaAffx.11840.1.S1_at | AW349211 | Post-Transcription | | | Predicted E3 |
| | | | | | | ubiquitin ligase |
| 46 | GmaAffx.60958.3.S1_at | BG881523 | No Homology to | | | |
| | | | Known Proteins | | | |
| 47 | GmaAffx.6818.1.A1_at | BU546572 | Metabolism | | | Iron/ascorbate |
| | | | | | | family |
| | | | | | | oxidoreductases |
| 48 | GmaAffx.80638.1.S1_at | AW396070 | Unclassified - | | | |
| | | | Hypothetical Protein | | | |
| | | | NOT Supported by | | | |
| | | | cDNA | | | |
| 49 | Gma.5621.1.S1_at | BU579234 | Unclassified - | | | |
| | | | Hypothetical Protein | | | |
| | | | NOT Supported by | | | |
| | | | cDNA | | | |
| 50 | GmaAffx.93592.1.S1_s_at | CF809317 | Unclassified - | | | |
| | | | Hypothetical Protein | | | |
| | | | Supported by | | | |
| | | | cDNA | | | |
| 51 | GmaAffx.77190.1.S1_at | BQ741890 | Metabolism | | | |
| 52 | GmaAffx.67456.1.S1_at | BM893057 | No Homology to | | | |
| | | | Known Proteins | | | |
| 53 | GmaAffx.11419.1.S1_at | AI900831 | No Homology to | | | |
| | | | Known Proteins | | | |
| 54 | GmaAffx.81692.1.S1_at | CA937041 | No Homology to | | | |
| | | | Known Proteins | | | |
| 55 | Gma.197.1.S1_at | U55874.1 | Metabolism | Amino Acid | | Asparagine |
| | | | | Biosynthesis/ | | synthase (glutamine- |
| | | | | Metabolism | | hydrolyzing) |
| 56 | GmaAffx.87550.1.S1_at | BM954718 | Disease & Defense | | | |
| 57 | Gma.6803.2.S1_s_at | BU082740 | Disease & Defense | | | |
| 58 | GmaAffx.78790.1.S1_at | BQ298762 | Transporter | | | P-type ATPase |
| 59 | GmaAffx.93534.1.S1_s_at | CF809259 | Unclassified - | | | |
| | | | Protein with | | | |
| | | | Unknown Function | | | |
| 60 | GmaAffx.47788.1.A1_at | CD408318 | Metabolism | | | Aldehyde |
| | | | | | | dehydrogenase |
| 61 | Gma.10857.2.S1_s_at | BQ612778 | Unclassified - | | | Fasciclin |
| | | | Hypothetical Protein | | | and related |
| | | | Supported by | | | adhesion |
| | | | cDNA | | | glycoproteins |
| 62 | GmaAffx.87721.1.S1_at | AW156448 | Intracellular | | | Rho GDP-dissocia- |
| | | | Traffic | | | tion inhibitor |
| 63 | Gma.13613.1.A1_at | CD401987 | No Homology to | | | |
| | | | Known Proteins | | | |
| 64 | Gma.9013.1.S1_x_at | AF243368.1 | Disease & Defense | | | Glutathione |
| | | | | | | S-transferase |
| 65 | Gma.3400.1.S1_at | BQ627988 | No Homology to | | | |
| | | | Known Proteins | | | |
| 66 | Gma.10940.1.S1_at | BU550016 | Unclassified - | | | Metalloendopeptidase |
| | | | Hypothetical Protein | | | family - saccharolysin |
| | | | Supported by | | | & thimet |
| | | | cDNA | | | oligopeptidase |
| 67 | GmaAffx.53033.1.S1_at | BG046837 | Transcription | Transcription | AP2/ | |
| | | | | Factor | EREBP | |
| 68 | Gma.2771.1.S1_at | AW309763 | Unclassified - | | | |
| | | | Hypothetical Protein | | | |
| | | | Supported by | | | |
| | | | cDNA | | | |
| 69 | Gma.10701.2.S1_at | AW101512 | No Homology to | | | |
| | | | Known Proteins | | | |
| 70 | GmaAffx.53585.1.S1_at | B1471867 | Protein | | | Molecular chaperone |
| | | | Destination | | | (DnaJ superfamily) |
| | | | & Storage | | | |
| 71 | GmaAffx.77622.1.S1_at | B1674271 | Transcription | Transcription | Zinc | |
| | | | | Factor | Finger | |
| 72 | GmaAffx.2412.1.S1_at | AI961002 | Secondary | GA | | Iron/ascorbate family |
| | | | Metabolism | Biosynthesis | | oxidoreductases |
| 73 | GmaAffx.88251.1.S1_s_at | BU964415 | Disease & Defense | | | Soluble epoxide |
| | | | | | | hydrolase |
| 74 | GmaAffx.68318.1.S1_at | BU545357 | Transcription | Transcription | AUX/ | |
| | | | | Factor | IAA | |
| 75 | Gma.17798.1.S1_at | BU084728 | Unclassified - | | | |
| | | | Hypothetical Protein | | | |
| | | | Supported by | | | |
| | | | cDNA | | | |
| 76 | Gma.11341.3.S1_s_at | BM177332 | Unclassified - | | | |
| | | | Hypothetical Protein | | | |
| | | | NOT Supported by | | | |
| | | | cDNA | | | |
| 77 | GmaAffx.90097.1.S1_at | CF805822 | Cell Structure | | | |
| 78 | Gma.15499.1.S1_at | AW348149 | Energy | | | |
| 79 | GmaAffx.83232.1.S1_at | BE023128 | Disease & Defense | | | |
| 80 | Gma.3160.1.S1_at | BG507816 | Energy | | | Thioredoxin |
| 81 | Gma.8525.1.S1_s_at | A1966178 | Disease & Defense | | | |
| 82 | Gma.11672.1.S1_at | BF425929 | Unclassified - | | | |
| | | | Hypothetical Protein | | | |
| | | | Supported by | | | |
| | | | cDNA | | | |
| 83 | Gma.385.1.S1_at | BE347563 | Intracellular | | | Annexin |
| | | | Traffic | | | |
| 84 | Gma.11135.1.S1_at | AI856724 | Energy | | | ATP-citrate lyase |
| 85 | Gma.1445.1.S1_a_at | BQ454241 | Metabolism | | | Flavonol reductase/ |
| | | | | | | cinnamoyl-CoA |
| | | | | | | reductase |
| 86 | GmaAffx.35729.2.S1_at | BM732483 | Unclassified - | | | |
| | | | Hypothetical Protein | | | |
| | | | Supported by | | | |
| | | | cDNA | | | |
| 87 | Gma.13257.1.A1_at | CD396346 | Unclassified - | | | |
| | | | Protein with | | | |
| | | | Unknown Function | | | |
| 88 | Gma.9228.1.S1_at | CD417435 | Unclassified - | | | |
| | | | Hypothetical Protein | | | |
| | | | NOT Supported by | | | |
| | | | cDNA | | | |
| 89 | Gma.1749.3.S1_at | BI784667 | Protein | Ubiquitin | | FOG: |
| | | | Destination | Degradation | | Predicted E3 |
| | | | & Storage | | | ubiquitin ligase |
| 90 | GmaAffx.45853.1.S1_at | BQ081229 | Unclassified - | | | |
| | | | Protein with | | | |
| | | | Unknown Function | | | |
| 91 | Gma.14563.1.S1_s_at | CD399224 | Energy | | | |
| 92 | GmaAffx.74500.1.A1_at | BU551117 | No Homology to | | | |
| | | | Known Proteins | | | |
| 93 | Gma.16288.1.S1_at | AW598590 | Unclassified - | | | Fasciclin and |
| | | | Hypothetical Protein | | | related adhesion |
| | | | NOT Supported by | | | glycoproteins |
| | | | cDNA | | | |
| 94 | GmaAffx.85342.1.S1_at | BI426916 | Unclassified - | | | |
| | | | Hypothetical Protein | | | |
| | | | NOT Supported by | | | |
| | | | cDNA | | | |
| 95 | GmaAffx.15471.3.S1_s_at | BE346777 | Transcription | Transcription | MYB | Transcription factor, |
| | | | | Factor | | Myb superfamily |
| 96 | Gma.17861.1.S1_at | AW310110 | Cell Structure | | | |
| 97 | Gma.1294.1.S1_at | CD395653 | Unclassified - | | | |
| | | | Hypothetical Protein | | | |
| | | | Supported by | | | |
| | | | cDNA | | | |
| 98 | GmaAffx.36017.1.S1_at | BG154593 | Unclassified - | | | |
| | | | Hypothetical Protein | | | |
| | | | Supported by | | | |
| | | | cDNA | | | |
| 99 | Gma.2470.1.S1_s_at | A1442629 | Cell Structure | | | |
| 100 | GmaAffx.91514.1.S1_at | CF807239 | No Homology to | | | |
| | | | Known Proteins | | | |
| 101 | GmaAffx.28690.1.S1_at | CA799411 | Transcription | Transcription | Homeo- | |
| | | | | Factor | domain | |
| 102 | Gma.17763.1.A1_at | BI321757 | Unclassified - | | | Diadenosine and |
| | | | Hypothetical Protein | | | diphosphoinositol |
| | | | Supported by | | | polyphosphate |
| | | | cDNA | | | phosphohydrolase |
| 103 | Gma.6659.1.S1_at | BQ629137 | Unclassified - | | | Protein involved |
| | | | Protein with | | | in membrane traffic |
| | | | Unknown Function | | | (YOP1/TB2/DP1/ |
| | | | | | | HVA22 family) |
| 104 | Gma.8143.1.S1_at | BG725425 | Signal | MAPK | | Mitogen-activated |
| | | | Transduction | | | protein kinase |
| 105 | GmaAffx.68099.1.S1_at | AW734055 | No Homology to | | | |
| | | | Known Proteins | | | |
| 106 | Gma.3065.1.S1_s_at | AW349564 | Unclassified - | | | |
| | | | Hypothetical Protein | | | |
| | | | Supported by | | | |
| | | | cDNA | | | |
| 107 | Gma.3881.1.S1_at | X92437.1 | Metabolism | Cytochrome | CYP73A | Cytochrome P450 |
| | | | | P450 | | CYP2 subfamily |
| 108 | Gma.5468.2.S1_a_at | BE802025 | Metabolism | | | Beta-galactosidase |
| 109 | GmaAffx.87295.1.S1_at | BM519660 | No Homology to | | | |
| | | | Known Proteins | | | |
| 110 | Gma.1701.1.S1_at | M67449.1 | Signal | MAPKKK | Raf | Tyrosine kinase |
| | | | Transduction | | | specific for activated |
| | | | | | | (GTP-bound) |
| | | | | | | p21cdc42Hs |
| 111 | GmaAffx.3548.1.S1_at | CA851347 | Transporter | | | Apolipoprotein |
| | | | | | | D/Lipocalin |
| 112 | Gma.12584.2.A1_at | CD406878 | Metabolism | | | Long-chain acyl-CoA |
| | | | | | | synthetases |
| | | | | | | (AMP-forming) |
| 113 | GmaAffx.8113.1.S1_at | BE659323 | Disease & Defense | | | |
| 114 | GmaAffx.64885.1.S1_at | BQ081653 | No Homology to | | | |
| | | | Known Proteins | | | |
| 115 | Gma.1445.3.S1_a_at | BE805686 | Metabolism | | | Flavonol reductase/ |
| | | | | | | cinnamoyl-CoA |
| | | | | | | reductase |
| 116 | GmaAffx.93308.1.S1_at | CF809033 | Protein | | | Glutathione |
| | | | Destination | | | S-transferase |
| | | | & Storage | | | |
| 117 | Gma.8072.1.S1_a_at | BQ627840 | Transcription | Transcription | Basic | |
| | | | | Factor | Leucine | |
| | | | | | Zipper | |
| | | | | | (bZlP) | |
| 118 | GmaAffx.68588.1.S1_at | B1787152 | Unclassified - | | | |
| | | | Hypothetical Protein | | | |
| | | | Supported by | | | |
| | | | cDNA | | | |
| 119 | GmaAffx.21943.1.S1_at | AW101735 | Unclassified - | | | |
| | | | Hypothetical Protein | | | |
| | | | NOT Supported by | | | |
| | | | cDNA | | | |
| 120 | GmaAffx.24382.1.S1_at | BM886764 | No Homology to | | | |
| | | | Known Proteins | | | |
| 121 | GmaAffx.62941.1.S1_at | AW597498 | Unclassified - | | | |
| | | | Protein with | | | |
| | | | Unknown Function | | | |
| 122 | GmaAffx.80474.1.S1_at | AW707170 | Signal | MAPKKK | MEKK | MEKK and related |
| | | | Transduction | | | serine/threonine |
| | | | | | | protein kinases |
| 123 | GmaAffx.84721.2.S1_s_at | BG041982 | Metabolism | | | Hydroxymethylglutaryl- |
| | | | | | | CoA lyase |
| 124 | GmaAffx.53069.1.S1_at | AW733700 | Unclassified - | | | |
| | | | Hypothetical Protein | | | |
| | | | NOT Supported by | | | |
| | | | cDNA | | | |
| 125 | GmaAffx.18236.1.S1_at | BG790288 | No Homology to | | | |
| | | | Known Proteins | | | |
| 126 | Gma.13458.1.A1_at | CD398733 | Protein | Ubiquitin | | FOG: Predicted E3 |
| | | | Destination | Degradation | | ubiquitin ligase |
| | | | & Storage | | | |
| 127 | GmaAffx.51044.1.S1_at | BM522670 | Unclassified - | | | Copine |
| | | | Hypothetical Protein | | | |
| | | | Supported by | | | |
| | | | cDNA | | | |
| 128 | Gma.10520.1.A1_at | CD398227 | Unclassified - | | | |
| | | | Hypothetical Protein | | | |
| | | | Supported by | | | |
| | | | cDNA | | | |
| 129 | GmaAffx.85565.1.S1_at | BE611082 | Signal | Calcium | Calcium | Ca2+/calmodulin- |
| | | | Transduction | Binding | Dependent | dependent protein |
| | | | | | Protein | kinase, EF-Hand |
| | | | | | Kinase | protein superfamily |
| 130 | GmaAffx.78768.1.S1_s_at | CA784898 | No Homology to | | | |
| | | | Known Proteins | | | |
| 131 | Psojae_rRNA_482_at | | | | | |
| 132 | Gma.14611.1.S1_at | CA852673 | Unclassified - | | | |
| | | | Protein with | | | |
| | | | Unknown Function | | | |
| 133 | GmaAffx.50617.1.S1_at | AW101420 | Unclassified - | | | |
| | | | Hypothetical Protein | | | |
| | | | Supported by | | | |
| | | | cDNA | | | |
| 134 | GmaAffx.93017.1.S1_at | CF808742 | Disease & Defense | | | Soluble epoxide |
| | | | | | | hydrolase |
| 135 | Gma.4673.1.A1_at | BG508468 | No Homology to | | | |
| | | | Known Proteins | | | |
| 136 | GmaAffx.21773.1.S1_at | BU763882 | Unclassified - | | | |
| | | | Hypothetical Protein | | | |
| | | | NOT Supported by | | | |
| | | | cDNA | | | |
| 137 | Gma.15563.1.S1_at | CD415760 | Unclassified - | | | |
| | | | Hypothetical Protein | | | |
| | | | Supported by | | | |
| | | | cDNA | | | |
| 138 | GmaAffx.10297.1.S1_at | BE475510 | Metabolism | | | Beta-galactosidase |
| 139 | Gma.9842.1.S1_at | BM528240 | Unclassified - | | | Leucine rich repeat |
| | | | Hypothetical Protein | | | proteins, some |
| | | | Supported by | | | proteins contain |
| | | | cDNA | | | F-box |
| 140 | GmaAffx.18113.2.S1_x_at | AW706795 | Unclassified - | Hormone | | |
| | | | Protein with | Induced | | |
| | | | Unknown Function | | | |
| 141 | GmaAffx.90853.1.S1_at | CF806578 | No Homology to | | | |
| | | | Known Proteins | | | |
| 142 | Gma.13042.1.S1_at | CD390874 | Unclassified - | | | |
| | | | Hypothetical Protein | | | |
| | | | NOT Supported by | | | |
| | | | cDNA | | | |
| 143 | Gma.6804.1.S1_at | BE657581 | Protein | | | Aspartyl protease |
| | | | Destination | | | |
| | | | & Storage | | | |
| 144 | Gma.17136.1.S1_s_at | BE022719 | Energy | | | Cytochrome b |
| 145 | GmaAffx.92463.1.S1_at | CF808188 | Metabolism | | | NADP+-dependent |
| | | | | | | malic enzyme |
| 146 | GmaAffx.20688.1.S1_at | BG157357 | Unclassified - | | | Fatty acyl-CoA |
| | | | Protein with | | | elongase/Polyunsaturated |
| | | | Unknown Function | | | fatty acid specific |
| | | | | | | elongation enzyme |
| 147 | GmaAffx.81388.1.S1_at | BF424571 | Signal Transduction | Protein | | Serine/threonine |
| | | | | Kinase | | protein kinase |
| 148 | GmaAffx.6017.2.S1_at | B1469662 | Unclassified - | | | |
| | | | Hypothetical Protein | | | |
| | | | NOT Supported by | | | |
| | | | cDNA | | | |
| 149 | GmaAffx.91688.1.S1_at | CF807413 | No Homology to | | | |
| | | | Known Proteins | | | |
| 150 | GmaAffx.35278.1.S1_at | BE346991 | Disease & Defense | | | |
| 151 | GmaAffx.73088.1.S1_at | BE475110 | Unclassified - | | | |
| | | | Hypothetical Protein | | | |
| | | | Supported by | | | |
| | | | cDNA | | | |
| 152 | Gma.15487.1.S1_at | D63781.1 | Disease & Defense | | | Soluble epoxide hydrolase |
| 153 | Gma.4305.3.S1_a_at | BG044173 | Transporter | lon Channel | Cyclic | K+-channel ERG |
| | | | | Families | nucleotide | and related proteins, |
| | | | | | gated | contain PAS/PAC |
| | | | | | channel | sensor domain |
| | | | | | family | |
| 154 | GmaAffx.77097.1.S1_at | BG237643 | No Homology to | | | |
| | | | Known Proteins | | | |
| 155 | GmaAffx.18351.1.S1_at | B1472051 | Unclassified - | | | FOG: Armadillo/beta- |
| | | | Hypothetical Protein | | | catenin-like repeats |
| | | | Supported by | | | |
| | | | cDNA | | | |
| 156 | Gma.5418.2.S1_at | B1469627 | Cell Growth | | | |
| | | | & Division | | | |
| 157 | Gma.5218.1.A1_at | CD392232 | Metabolism | | | Predicted carbonic |
| | | | | | | anhydrase involved in |
| | | | | | | protection against |
| | | | | | | oxidative damage |
| 158 | Gma.415.1.A1_at | AW350315 | Protein | | | FOG: Predicted E3 |
| | | | Destination | | | ubiquitin ligase |
| | | | & Storage | | | |
| 159 | Gma.1123.1.S1_at | CD402207 | Unclassified - | | | |
| | | | Hypothetical Protein | | | |
| | | | Supported by | | | |
| | | | cDNA | | | |
| 160 | GmaAffx.26683.1.A1_at | BE820379 | No Homology to | | | |
| | | | Known Proteins | | | |
| 161 | GmaAffx.34775.1.S1_at | A1794730 | Unclassified - | | | FOG: Predicted E3 |
| | | | Protein with | | | ubiquitin ligase |
| | | | Unknown Function | | | |
| 162 | GmaAffx.17961.2.S1_at | BG511562 | Protein | | | Molecular |
| | | | Destination | | | chaperones HSP70/ |
| | | | & Storage | | | HSC70, HSP70 |
| | | | | | | superfamily |
| 163 | GmaAffx.9482.2.S1_at | A1900615 | Unclassified - | | | Predicted guanosine |
| | | | Hypothetical Protein | | | polyphosphate |
| | | | NOT Supported by | | | pyrophosphohydrolase/ |
| | | | cDNA | | | synthase |
| 164 | Gma.6245.1.S1_at | CA938361 | No Homology to | | | |
| | | | Known Proteins | | | |
| 165 | Gma.2486.1.S1_at | BF219523 | Unclassified - | | | Iron/ascorbate family |
| | | | Hypothetical Protein | | | oxidoreductases |
| | | | NOT Supported by | | | |
| | | | cDNA | | | |
| 166 | Gma.12494.1.S1_at | BG839922 | Protein | Ubiquitin | E3 | FOG: Predicted E3 |
| | | | Destination | Degradation | Ubiquitin | ubiquitin ligase |
| | | | & Storage | | Ligase | |
| 167 | GmaAffx.51468.1.S1_at | BQ628796 | No Homology to | | | |
| | | | Known Proteins | | | |
| 168 | Gma.17062.1.S1_at | AW277440 | No Homology to | | | |
| | | | Known Proteins | | | |
| 169 | Gma.892.1.A1_at | BE822836 | Unclassified - | | | Prolyl 4-hydroxylase |
| | | | Protein with | | | alpha subunit |
| | | | Unknown Function | | | |
| 170 | GmaAffx.74304.1.S1_at | BU577360 | Unclassified - | | | |
| | | | Hypothetical Protein | | | |
| | | | NOT Supported by | | | |
| | | | cDNA | | | |
| 171 | GmaAffx.81253.1.S1_at | BE807371 | Unclassified - | | | |
| | | | Protein with | | | |
| | | | Unknown Function | | | |
| 172 | GmaAffx.91108.1.S1_at | CF806833 | Unclassified - | | | |
| | | | Hypothetical Protein | | | |
| | | | Supported by | | | |
| | | | cDNA | | | |
| 173 | Gma.1190.1.S1_at | AF279267.1 | Disease & Defense | | | Acyl-CoA synthetase |
| 174 | Gma.10281.1.S1_at | CA852560 | Unclassified - | | | Predicted E3 |
| | | | Protein with | | | ubiquitin ligase |
| | | | Unknown Function | | | |
| 175 | Gma.4315.1.S1_at | BM886786 | Unclassified - | | | |
| | | | Protein with | | | |
| | | | Unknown Function | | | |
| 176 | GmaAffx.71944.1.S1_at | AW351155 | Unclassified - | | | |
| | | | Hypothetical Protein | | | |
| | | | Supported by | | | |
| | | | cDNA | | | |
| 177 | GmaAffx.91814.1.S1_at | CF807539 | Unclassified - | | | |
| | | | Hypothetical Protein | | | |
| | | | Supported by | | | |
| | | | cDNA | | | |
| 178 | Gma.15773.1.A1_at | CD406280 | Metabolism | | | |
| 179 | Gma.12873.1.S1_at | CD393774 | No Homology to | | | |
| | | | Known Proteins | | | |
| 180 | Gma.14312.1.S1_at | CA852635 | No Homology to | | | |
| | | | Known Proteins | | | |
| 181 | Gma.15470.1.S1_at | BU546303 | Unclassified - | | | |
| | | | Hypothetical Protein | | | |
| | | | Supported by | | | |
| | | | cDNA | | | |
| 182 | GmaAffx.4505.1.S1_at | AW306682 | Unclassified - | | | Predicted NUDIX |
| | | | Protein with | | | hydrolase FGF-2 and |
| | | | Unknown Function | | | related proteins |
| 182 | GmaAffx.4505.1.S1_at | AW306682 | Unclassified - | | | Cdc4 and related |
| | | | Protein with | | | F-box and WD-40 |
| | | | Unknown Function | | | proteins |
| 183 | GmaAffx.90827.1.S1_at | CF806552 | Unclassified - | | | Molecular chaperone |
| | | | Hypothetical Protein | | | (small heat-shock |
| | | | Supported by | | | protein Hsp26/Hsp42) |
| | | | cDNA | | | |
| 184 | GmaAffx.89971.1.S1_s_at | CF805696 | Intracellular | | | Annexin |
| | | | Traffic | | | |
| 185 | GmaAffx.66982.1.S1_at | BI970847 | Protein | | | Molecular chaperone |
| | | | Destination | | | (small heat-shock |
| | | | & Storage | | | protein Hsp26/Hsp42) |
| 186 | GmaAffx.45236.1.S1_at | BG156638 | Disease & Defense | | | Purple (tartrate- |
| | | | | | | resistant) acid |
| | | | | | | phosphatase |
| 187 | Gma.10795.1.S1_at | AI442845 | Cell Structure | | | |
| 188 | GmaAffx.55244.1.A1_at | BU549116 | No Homology to | | | |
| | | | Known Proteins | | | |
| 189 | GmaAffx.69792.1.S1_s_at | BU550596 | Transcription | Transcription | AP2/ | |
| | | | | Factor | EREBP | |
| 190 | Gma.16547.1.S1_at | BU545050 | Transcription | Transcription | WRKY | |
| | | | | Factor | | |
| 191 | GmaAffx.74211.1.S1_at | BE820853 | Protein | | | Aspartyl protease |
| | | | Destination | | | |
| | | | & Storage | | | |
| 192 | GmaAffx.42667.3.S1_at | AW306785 | Transcription | Transcription | GRAS | |
| | | | | Factor | | |
| 193 | Gma.2772.1.S1_at | BG238134 | No Homology to | | | |
| | | | Known Proteins | | | |
| 194 | GmaAffx.18742.1.S1_at | B1970450 | Unclassified - | | | Predicted membrane |
| | | | Hypothetical Protein | | | protein |
| | | | Supported by | | | |
| | | | cDNA | | | |
| 195 | Gma.1947.1.S1_at | BI968172 | Transcription | Transcription | Homeo- | Transcription factor |
| | | | | Factor | domain | ME1S1 and related |
| | | | | | | HOX domain proteins |
| 196 | Gma.11131.1.S1_at | BI968388 | Protein | | | Molecular chaperone |
| | | | Destination | | | (small heat-shock |
| | | | & Storage | | | protein Hsp26/Hsp42) |
| 197 | GmaAffx.72580.1.S1_at | BG157182 | Unclassified - | | | |
| | | | Hypothetical Protein | | | |
| | | | Supported by | | | |
| | | | cDNA | | | |
| 198 | Gma.4432.1.S1_a_at | BQ610841 | Unclassified - | Hormone | | Voltage-gated shaker- |
| | | | Protein with | Induced | | like K+ channel, |
| | | | Unknown Function | | | subunit beta/KCNAB |
| 199 | GmaAffx.62375.1.S1_at | BQ612727 | Unclassified - | | | |
| | | | Hypothetical Protein | | | |
| | | | Supported by | | | |
| | | | cDNA | | | |
| 200 | GmaAffx.38733.1.A1_at | BE657590 | No Homology to | | | |
| | | | Known Proteins | | | |
| 201 | GmaAffx.65580.1.S1_at | CA799171 | Metabolism | | | |
| 202 | GmaAffx.16201.1.S1_at | BU550725 | No Homology to | | | |
| | | | Known Proteins | | | |
| 203 | Gma.4755.1.S1_at | AW310232 | Unclassified - | | | |
| | | | Hypothetical Protein | | | |
| | | | NOT Supported by | | | |
| | | | cDNA | | | |
| 204 | GmaAffx.9059.1.S1_at | AW423327 | No Homology to | | | |
| | | | Known Proteins | | | |
| 205 | GmaAffx.51614.1.S1_at | BU089837 | No Homology to | | | |
| | | | Known Proteins | | | |
| 206 | Gma.153.1.S1_x_at | Y10491.1 | Metabolism | Cytochrome | CYP82C | Cytochrome P450 |
| | | | | P450 | | CYP2 subfamily |
| 207 | GmaAffx.47716.1.A1_at | CD397037 | Unclassified - | | | |
| | | | Hypothetical Protein | | | |
| | | | NOT Supported by | | | |
| | | | cDNA | | | |
| 208 | Gma.4839.1.A1_at | AW310019 | Metabolism | | | Branched chain |
| | | | | | | aminotransferase BCAT1, |
| | | | | | | pyridoxal phosphate enzymes |
| | | | | | | type 1V superfamily |
| 209 | Gma.10969.1.S1_x_at | U36191.1 | Metabolism | | | |
| 210 | GmaAffx.5232.1.S1_at | BM309521 | Unclassified - | | | |
| | | | Protein with | | | |
| | | | Unknown Function | | | |
| 211 | GmaAffx.59762.1.S1_at | BI971811 | Unclassified - | | | |
| | | | Protein with | | | |
| | | | Unknown Function | | | |
| 212 | Gma.18020.1.S1_at | AW309711 | Signal | Protein | | Serine/threonine |
| | | | Transduction | Kinase | | protein kinase |
| 213 | GmaAffx.10580.1.S1_at | BF424010 | Transporter | | | Amino acid |
| | | | | | | ransporters |
| 214 | GmaAffx.12369.1.A1_at | CD412820 | Unclassified - | | | |
| | | | Hypothetical Protein | | | |
| | | | Supported by | | | |
| | | | cDNA | | | |
| 215 | Gma.5256.2.S1_at | AI441316 | Unclassified - | | | |
| | | | Hypothetical Protein | | | |
| | | | NOT Supported by | | | |
| | | | cDNA | | | |
| 216 | GmaAffx.1890.1.S1_s_at | B1970941 | Transcription | Transcription | Heat | Heat shock |
| | | | | Factor | Shock | transcription |
| | | | | | | factor |
| 217 | Gma.10266.1.S1_at | BG157207 | No Homology to | | | |
| | | | Known Proteins | | | |
| 218 | Gma.17535.1.S1_at | BM178659 | Unclassified - | | | Uncharacterized |
| | | | Hypothetical Protein | | | conserved protein, |
| | | | Supported by | | | contains double-stranded |
| | | | cDNA | | | beta-helix domain |
| 219 | Gma.4359.1.S1_at | A1794712 | Energy | | | Thioredoxin |
| 220 | Gma.7836.2.S1_at | BI425718 | Unclassified - | | | |
| | | | Hypothetical Protein | | | |
| | | | NOT Supported by | | | |
| | | | cDNA | | | |
| 221 | Gma.17502.2.S1_at | BG046201 | Transcription | Transcription | Unclassified | Zuotin and related |
| | | | | Factor | | molecular chaperones |
| | | | | | | (DnaJ superfamily), |
| | | | | | | contains DNA- |
| | | | | | | binding domains |
| 222 | Gma.4839.2.S1_at | B1974385 | Metabolism | | | Branched chain |
| | | | | | | aminotransferase |
| | | | | | | BCAT1, pyridoxal |
| | | | | | | phosphate enzymes type |
| | | | | | | 1V superfamily |
| 223 | Gma.11028.1.S1_at | BE823084 | Metabolism | | | Glyoxylate/hydroxypyruvate |
| | | | | | | reductase (D-isomer- |
| | | | | | | specific 2-hydroxy acid |
| | | | | | | dehydrogenase superfamily) |
| 224 | GmaAffx.91108.1.S1_s_at | CF806833 | Unclassified - | | | |
| | | | Hypothetical Protein | | | |
| | | | Supported by | | | |
| | | | cDNA | | | |
| 225 | Gma.5610.1.S1_at | AW348190 | Unclassified - | | | |
| | | | Hypothetical Protein | | | |
| | | | Supported by | | | |
| | | | cDNA | | | |
| 226 | GmaAffx.60673.1.S1_at | BU080770 | Unclassified - | | | |
| | | | Hypothetical Protein | | | |
| | | | NOT Supported by | | | |
| | | | cDNA | | | |
| 227 | Gma.4455.2.S1_at | BE059560 | Signal | Protein | | Serine/threonine |
| | | | Transduction | Kinase | | protein kinase |
| 228 | GmaAffx.26299.1.S1_at | BQ299016 | Signal | Protein | | |
| | | | Transduction | Kinase | | |
| 229 | Gma.13241.1.S1_at | CD395739 | Unclassified - | | | |
| | | | Hypothetical Protein | | | |
| | | | NOT Supported by | | | |
| | | | cDNA | | | |
| 230 | Gma.17806.1.S1_at | BE820775 | Transporter | | | H+/oligopeptide |
| | | | | | | symporter |
| 231 | Gma.14295.1.S1_at | CA852253 | No Homology to | | | |
| | | | Known Proteins | | | |
| 232 | Gma.17642.1.S1_at | BU550265 | Unclassified - | | | BP1/LBP/CETP |
| | | | Hypothetical Protein | | | family protein |
| | | | Supported by | | | |
| | | | cDNA | | | |
| 233 | GmaAffx.73303.1.S1_at | BE190156 | Unclassified - | | | Predicted membrane |
| | | | Hypothetical Protein | | | protein |
| | | | Supported by | | | |
| | | | cDNA | | | |
| 234 | Gma.7487.2.A1_at | AW349599 | Transporter | | | Amino acid |
| | | | | | | transporters |
| 235 | GmaAffx.7738.1.S1_s_at | BE058056 | No Homology to | | | |
| | | | Known Proteins | | | |
| 236 | Gma.4124.1.S1_at | BI787263 | Transcription | Transcription | Homeo- | |
| | | | | Factor | domain | |
| 237 | GmaAffx.89141.1.S1_at | CK605765 | Cell Structure | | | O-methyltransferase |
| 238 | Gma.7051.1.A1_at | BU544101 | Disease & Defense | | | Metacaspase involved |
| | | | | | | in regulation of |
| | | | | | | apoptosis |
| 239 | GmaAffx.24809.1.S1_at | CA820582 | Unclassified - | | | |
| | | | Hypothetical Protein | | | |
| | | | NOT Supported by | | | |
| | | | cDNA | | | |
| 240 | Gma.1114.1.S1_s_at | CD403139 | Unclassified - | | | |
| | | | Hypothetical Protein | | | |
| | | | NOT Supported by | | | |
| | | | cDNA | | | |
| 241 | GmaAffx.92230.1.S1_at | CF807302 | Disease & Defense | | | |
| 242 | GmaAffx.10590.1.S1_s_at | BU551319 | Signal | Protein | | |
| | | | Transduction | Kinase | | |
| 243 | GmaAffx.84607.2.S1_at | BU080867 | Unclassified - | | | |
| | | | Hypothetical Protein | | | |
| | | | Supported by | | | |
| | | | cDNA | | | |
| 244 | GmaAffx.54085.1.S1_at | BI970762 | Unclassified - | | | |
| | | | Hypothetical Protein | | | |
| | | | Supported by | | | |
| | | | cDNA | | | |
| 245 | GmaAffx.1694.1.S1_at | BQ454286 | Transcription | Transcription | MYB | |
| | | | | Factor | | |
| 246 | GmaAffx.60479.4.S1_at | BG507369 | Transcription | Transcription | Homeo- | Transcription factor |
| | | | | Factor | domain | MEIS1 and related |
| | | | | | | HOX domain proteins |
| 247 | GmaAffx.6824.2.S1_at | BQ094655 | Cell Structure | | | |
| 248 | Gma.3589.1.S1_at | AW101438 | Secondary | GA | | Iron/ascorbate family |
| | | | Metabolism | Biosynthesis | | oxidoreductases |
| 249 | Gma.5701.2.S1_at | BI701059 | Unclassified - | | | |
| | | | Hypothetical Protein | | | |
| | | | Supported by | | | |
| | | | cDNA | | | |
| 250 | Gma.10885.1.S1_at | BQ610245 | Unclassified - | | | Predicted kinase |
| | | | Hypothetical Protein | | | |
| | | | Supported by | | | |
| | | | cDNA | | | |
| 251 | Gma.10626.3.S1_at | BE022458 | Secondary | | | Iron/ascorbate family |
| | | | Metabolism | | | oxidoreductases |
| 252 | Gma.5038.1.A1_at | BE023631 | Disease & Defense | | | Acyl-CoA synthetase |
| 253 | GmaAffx.43786.2.S1_at | BU080930 | Signal | Histidine | | Sensory transduction |
| | | | Transduction | Kinase | | histidine kinase |
| 254 | GmaAffx.24844.1.S1_at | BU765373 | Unclassified - | | | Uncharacterized |
| | | | Hypothetical Protein | | | conserved protein |
| | | | Supported by | | | |
| | | | cDNA | | | |
| 255 | GmaAffx.27787.1.A1_at | BG789758 | Transporter | | | Transporter, ABC |
| | | | | | | superfamily (Breast |
| | | | | | | cancer resistance |
| | | | | | | protein) |
| 256 | Gma.1380.1.S1_at | CA935076 | Metabolism | | | |
| 257 | GmaAffx.74693.1.S1_at | CD406171 | Metabolism | | | |
| 258 | Gma.5709.1.S1_at | BG157556 | Unclassified - | | | |
| | | | Protein with | | | |
| | | | Unknown Function | | | |
| 259 | GmaAffx.22297.2.S1_at | BM271210 | Transcription | Transcription | WRKY | |
| | | | | Factor | | |
| 260 | Gma.5578.2.S1_a_at | AW310579 | Metabolism | Cytochrome | CYP98A | Cytochrome P450 |
| | | | | P450 | | CYP2 subfamily |
| 261 | GmaAffx.13730.1.S1_at | BI316755 | Transcription | Transcription | Basic | |
| | | | | Factor | Helix- | |
| | | | | | Loop- | |
| | | | | | Helix | |
| | | | | | (bHLH) | |
| 262 | GmaAffx.28870.1.S1_at | BG726532 | Unclassified - | | | |
| | | | Hypothetical Protein | | | |
| | | | Supported by | | | |
| | | | cDNA | | | |
| 263 | GmaAffx.73195.1.S1_at | BI701045 | Secondary | Flavonoid | | Flavonol reductase/ |
| | | | Metabolism | Biosynthesis | | cinnamoyl-CoA |
| | | | | | | reductase |
| 264 | Gma.16147.1.S1_at | BE473577 | Transcription | Transcription | Basic | |
| | | | | Factor | Helix- | |
| | | | | | Loop- | |
| | | | | | Helix | |
| | | | | | (bHLH) | |
| 265 | GmaAffx.37636.1.S1_at | BQ610347 | Metabolism | Amino Acid | | Alanine-glyoxylate |
| | | | | Biosynthesis/ | | aminotransferase |
| | | | | Metabolism | | AGT2 |
| 266 | Gma.7047.1.S1_s_at | CD417401 | Unclassified - | | | |
| | | | Protein with | | | |
| | | | Unknown Function | | | |
| 267 | Gma.I649.1.AI_at | BE659535 | No Homology to | | | |
| | | | Known Proteins | | | |
| 268 | GmaAffx.8304.1.A1_at | BE822695 | Unclassified - | | | Possible |
| | | | Hypothetical Protein | | | oxidoreductase |
| | | | Supported by | | | |
| | | | cDNA | | | |
| 269 | GmaAffx.3726.1.A1_at | BE822942 | Unclassified - | | | |
| | | | Hypothetical Protein | | | |
| | | | Supported by | | | |
| | | | cDNA | | | |
| 270 | GmaAffx.82034.1.S1_at | BM887093 | Transcription | Transcription | Homeo- | Transcription factor |
| | | | | Factor | domain | HEX, contains HOX |
| | | | | | | and HALZ domains |
| 271 | Gma.16336.I.S1_at | BE659555 | Protein | | | Predicted pilin-like |
| | | | Destination | | | transcription factor |
| | | | & Storage | | | |
| 272 | Gma.16276.2.S1_a_at | BG316414 | Unclassified - | | | FOG: PPR repeat |
| | | | Hypothetical Protein | | | |
| | | | Supported by | | | |
| | | | cDNA | | | |
| 273 | Gma.13146.1.S1_at | BM731716 | Unclassified - | | | |
| | | | Hypothetical Protein | | | |
| | | | Supported by | | | |
| | | | cDNA | | | |
| 274 | GmaAffx.86020.1.S1_at | BI701699 | Signal Transduction | Protein | AtCIPKs | Serine/threonine |
| | | | | Kinase | | protein kinase |
| 275 | GmaAffx.68853.1.S1_at | CA803I08 | Unclassified - | | | |
| | | | Hypothetical Protein | | | |
| | | | Supported by | | | |
| | | | cDNA | | | |
| 276 | GmaAffx.73021.1.S1_at | BE556340 | Disease & Defense | | | |
| 277 | GmaAffx.80899.1.S1_at | AW348263 | Unclassified - | | | |
| | | | Hypothetical Protein | | | |
| | | | Supported by | | | |
| | | | cDNA | | | |
| 278 | GmaAffx.77052.1.S1_at | BE556140 | Transporter | Inorganic | Putative | |
| | | | | Solute Co- | potassium | |
| | | | | transporters | transporter | |
| | | | | | family | |
| 279 | GmaAffx.77052.2.A1_at | BG155285 | Transporter | Inorganic | Putative | |
| | | | | Solute Co- | potassium | |
| | | | | transporters | transporter | |
| | | | | | family | |
| 280 | GmaAffx.17924.1.S1_at | BG157338 | Unclassified - | | | |
| | | | Protein with | | | |
| | | | Unknown Function | | | |
| 281 | Gma.6730.1.S1_at | BE211440 | Metabolism | | | UDP-glucuronosyl |
| | | | | | | and UDP-glucosyl |
| | | | | | | transferase |
| 282 | Gma.5038.2.S1_a_at | CD417606 | Disease & Defense | | | Acyl-CoA |
| | | | | | | synthetase |
| 283 | Gma.12817.1.A1_at | CD405335 | Unclassified - | | | |
| | | | Hypothetical Protein | | | |
| | | | Supported by | | | |
| | | | cDNA | | | |
| 284 | GmaAffx.31571.1.S1_at | BU551140 | Unclassified - | | | |
| | | | Hypothetical Protein | | | |
| | | | NOT Supported by | | | |
| | | | cDNA | | | |
| 285 | Gma.4990.1.A1_at | B1315863 | Unclassified - | | | Uncharacterized |
| | | | Hypothetical Protein | | | conserved protein |
| | | | Supported by | | | |
| | | | cDNA | | | |
| 286 | Gma.13340.1.S1_at | BM523855 | Metabolism | | | UDP-glucuronosyl |
| | | | | | | and UDP-glucosyl |
| | | | | | | transferase |
| 287 | GmaAffx.28196.2.A1_s_at | CD416781 | Metabolism | | | Long-chain acyl-CoA |
| | | | | | | synthetases (AMP- |
| | | | | | | forming) |
| 288 | GmaAffx.90585.1.S1_s_at | CF806310 | Protein | | | Aspartyl protease |
| | | | Destination | | | |
| | | | & Storage | | | |
| 289 | GmaAffx.62856.1.S1_at | AW432978 | Signal | MAPKKK | ZlK | Serine/threonine |
| | | | Transduction | | | protein kinase |
| 290 | Gma.8706.1.S1_at | BU762562 | Unclassified - | | | |
| | | | Hypothetical Protein | | | |
| | | | Supported by | | | |
| | | | cDNA | | | |
| 291 | GmaAffx.39467.1.S1_at | BQ299569 | Unclassified - | | | |
| | | | Hypothetical Protein | | | |
| | | | NOT Supported by | | | |
| | | | cDNA | | | |
| 292 | Gma.4049.1.S1_at | BQ786519 | Signal | Protein | | Serine/threonine |
| | | | Transduction | Kinase | | protein kinase |
| 293 | GmaAffx.38961.1.S1_at | BU081740 | Transcription | Transcription | MYB | Transcription factor, |
| | | | | Factor | | Myb superfamily |
| 294 | Gma.5691.1.S1_at | BM522643 | No Homology to | | | |
| | | | Known Proteins | | | |
| 295 | Gma.5578.3.S1_at | AW310572 | Metabolism | Cytochrome | CYP98A | Cytochrome P450 CYP2 |
| | | | | P450 | | subfamily |
| 296 | GmaAffx.85878.1.S1_at | BF067810 | Intracellular | | | Aquaporin |
| | | | Traffic | | | (major intrinsic |
| | | | | | | protein family) |
| 297 | GmaAffx.61571.2.S1_at | AI939190 | Unclassified - | | | |
| | | | Hypothetical Protein | | | |
| | | | NOT Supported by | | | |
| | | | cDNA | | | |
| 298 | Gma.2831.1.S1_s_at | AW349413 | Transcription | Transcription | Zinc | |
| | | | | Factor | Finger | |
| 299 | Gma.559.1.A1_at | BQ630327 | Protein | | | |
| | | | Destination | | | |
| | | | & Storage | | | |
| 300 | GmaAffx.38558.1.S1_at | CD407094 | Metabolism | | | NADP+-dependent |
| | | | | | | malic enzyme |
| 301 | GmaAffx.2745.2.A1_at | BQ611985 | Unclassified - | | | Acetyl- |
| | | | Hypothetical Protein | | | glucosaminyltransferase |
| | | | Supported by | | | EXT1/exostosin 1 |
| | | | cDNA | | | |
| 302 | GmaAffx.91254.1.S1_at | CF806979 | Unclassified - | | | |
| | | | Hypothetical Protein | | | |
| | | | Supported by | | | |
| | | | cDNA | | | |
| 303 | GmaAffx.46770.1.S1_at | BQ298902 | Transporter | lon Channel | Putative | Glutamate-gated |
| | | | | Families | ligand-gated | kainate-type |
| | | | | | ion channel | ion channel |
| | | | | | subunit | receptor subunit |
| | | | | | family | GluR5 and related |
| | | | | | | subunits |
| 304 | GmaAffx.1620.1.S1_at | AW424105 | Unclassified - | | | |
| | | | Hypothetical Protein | | | |
| | | | NOT Supported by | | | |
| | | | cDNA | | | |
| 305 | GmaAffx.64644.1.S1_at | AW348952 | Signal | Protein | | Serine/threonine |
| | | | Transduction | Kinase | | protein kinase |
| 306 | GmaAffx.79121.1.S1_at | CA801737 | Transcription | Transcription | Zinc | CCCH-type Zn-finger |
| | | | | Factor | Finger | protein |
| 307 | Gma.5578.3.S1_x_at | AW310572 | Metabolism | Cytochrome | CYP98A | Cytochrome P450 CYP2 |
| | | | | P450 | | subfamily |
| 308 | GmaAffx.92462.1.S1_at | CF808187 | Transcription | Transcription | Zinc | FOG: Predicted E3 |
| | | | | Factor | Finger | ubiquitin ligase |
| 309 | Gma.10615.1.A1_at | BU545626 | Transcription | Transcription | Basic | |
| | | | | Factor | Helix- | |
| | | | | | Loop- | |
| | | | | | Helix | |
| | | | | | (bHLH) | |
| 310 | GmaAffx.61272.1.S1_at | B [symbol] 972394 | No Homology to | | | |
| | | | Known Proteins | | | |
| 311 | GmaAffx.6008.1.S1_at | BU550119 | Transcription | Transcription | Basic | |
| | | | | Factor | Helix- | |
| | | | | | Loop- | |
| | | | | | Helix | |
| | | | | | (bHLH) | |
| 312 | GmaAffx.44716.1.A1_at | B1969342 | Disease & Defense | | | |
| 313 | GmaAffx.2745.1.S1_at | BF009213 | Unclassified - | | | Acetyl- |
| | | | Hypothetical Protein | | | glucosaminyltransferase |
| | | | Supported by | | | EXT1/exostosin 1 |
| | | | cDNA | | | |
| 314 | Gma.5104.1.S1_at | BE611202 | Unclassified - | | | |
| | | | Hypothetical Protein | | | |
| | | | Supported by | | | |
| | | | cDNA | | | |
| 315 | Gma.10331.1.A1_at | CD402553 | Unclassified - | | | Macrophage migration |
| | | | Protein with | | | inhibitory factor |
| | | | Unknown Function | | | |
| 316 | GmaAffx.91917.1.S1_s_at | CF807642 | Unclassified - | | | |
| | | | Hypothetical Protein | | | |
| | | | Supported by | | | |
| | | | cDNA | | | |
| 317 | GmaAffx.4445.1.A1_at | BU547894 | Transcription | Transcription | AP2/ | |
| | | | | Factor | EREBP | |
| 318 | GmaAffx.86613.2.S1_at | BE024005 | Metabolism | | | Glutaredoxin and |
| | | | | | | related proteins |
| 319 | GmaAffx.87493.1.S1_at | AW781752 | Unclassified - | | | |
| | | | Hypothetical Protein | | | |
| | | | NOT Supported by | | | |
| | | | cDNA | | | |
| 320 | GmaAffx.7771.1.S1_at | BE022913 | Transporter | Inorganic | Putative | Sulfate/bicarbonate/ |
| | | | | Solute Co- | sulfate | oxalate exchanger |
| | | | | transporters | transporter | SAT-1 and related |
| | | | | | family | transporters (SLC26 |
| | | | | | | family) |
| 321 | GmaAffx.36972.1.S1_at | BQ079450 | Unclassified - | | | |
| | | | Hypothetical Protein | | | |
| | | | NOT Supported by | | | |
| | | | cDNA | | | |
| 322 | Gma.1979.1.S1_a_at | CD403280 | Unclassified - | | | Multitransmembrane |
| | | | Protein with | | | protein |
| | | | Unknown Function | | | |
| 323 | GmaAffx.23475.1.A1_at | BU548049 | Unclassified - | | | |
| | | | Hypothetical Protein | | | |
| | | | Supported by | | | |
| | | | cDNA | | | |
| 324 | GmaAffx.68060.2.A1_at | AW396457 | No Homology to | | | |
| | | | Known Proteins | | | |
| 325 | Gma.3511.4.S1_at | BM526875 | Cell Structure | | | |
| 326 | Gma.13013.2.S1_a_at | AW457857 | Unclassified - | | | |
| | | | Hypothetical Protein | | | |
| | | | Supported by | | | |
| | | | cDNA | | | |
| 327 | GmaAffx.81399.1.S1_at | AW568772 | Cell Growth | | | |
| | | | & Division | | | |
| 328 | Gma.2342.1.S1_at | Z46953.1 | Transcription | Transcription | Heat | Heat shock |
| | | | | Factor | Shock | transcription factor |
| 329 | GmaAffx.74390.1.A1_at | BU544169 | Protein | | | Molecular chaperone |
| | | | Destination | | | (DnaJ superfamily) |
| | | | & Storage | | | |
| 330 | Gma.6836.1.A1_at | AW349121 | Unclassified - | | | Predicted pyroglutamyl |
| | | | Hypothetical Protein | | | peptidase |
| | | | Supported by | | | |
| | | | cDNA | | | |
| 331 | GmaAffx.23295.1.S1_at | B1968028 | Disease & Defense | | | |
| 332 | GmaAffx.17892.1.S1_at | A1442822 | Unclassified - | | | |
| | | | Hypothetical Protein | | | |
| | | | Supported by | | | |
| | | | cDNA | | | |
| 333 | GmaAffx.25598.1.S1_at | AW311255 | No Homology to | | | |
| | | | Known Proteins | | | |
| 334 | GmaAffx.84261.1.S1_at | B1701092 | Transcription | Transcription | AP2/ | |
| | | | | Factor | EREBP | |
| 335 | GmaAffx.82582.1.S1_at | BM139949 | No Homology to | | | |
| | | | Known Proteins | | | |
| 336 | Gma.11269.1.S1_at | CD395046 | Transporter | | | |
| 337 | Gma.1188.2.S1_at | BG406072 | Cell Growth | | | |
| | | | & Division | | | |
| 338 | GmaAffx.92127.1.S1_s_at | CF807852 | Transcription | Transcription | AP2/ | |
| | | | | Factor | EREBP | |
| 339 | GmaAffx.28397.1.S1_at | BM568229 | Metabolism | | | |
| 340 | GmaAffx.86675.1.S1_at | B1424294 | No Homology to | | | |
| | | | Known Proteins | | | |
| 341 | GmaAffx.5285.1.S1_at | BM523736 | Metabolism | | | |
| 342 | GmaAffx.84607.1.S1_at | BF596776 | Unclassified - | | | |
| | | | Hypothetical Protein | | | |
| | | | Supported by | | | |
| | | | cDNA | | | |
| 343 | GmaAffx.11857.1.A1_at | AW351021 | Unclassified - | | | |
| | | | Hypothetical Protein | | | |
| | | | Supported by | | | |
| | | | cDNA | | | |
| 344 | Gma.17007.1.S1_at | BE820533 | No Homology to | | | |
| | | | Known Proteins | | | |
| 345 | GmaAffx.15471.2.S1_at | B1784829 | Transcription | Transcription | MYB | Transcription factor, |
| | | | | Factor | | Myb superfamily |
| 346 | GmaAffx.74933.1.S1_at | CA935084 | Unclassified - | | | Aspartyl protease |
| | | | Protein with | | | |
| | | | Unknown Function | | | |
| 347 | GmaAffx.37458.2.S1_at | BQ079820 | Unclassified - | | | |
| | | | Hypothetical Protein | | | |
| | | | NOT Supported by | | | |
| | | | cDNA | | | |
| 348 | Gma.2602.1.S1_at | BU577743 | No Homology to | | | |
| | | | Known Proteins | | | |
| 349 | Gma.10701.1.S1_at | BF596612 | Unclassified - | | | |
| | | | Hypothetical Protein | | | |
| | | | Supported by | | | |
| | | | cDNA | | | |
| 350 | GmaAffx.13016.1.S1_at | BF069324 | No Homology to | | | |
| | | | Known Proteins | | | |
| 351 | Gma.8331.1.S1_at | BE659403 | Metabolism | | | Aldehyde dehydrogenase |
| 352 | Gma.11277.1.S1_at | CA802310 | Protein | | | |
| | | | Destination | | | |
| | | | & Storage | | | |
| 353 | Gma.10365.1.S1_at | BG725377 | Unclassified - | | | |
| | | | Hypothetical Protein | | | |
| | | | Supported by | | | |
| | | | cDNA | | | |
| 354 | GmaAffx.70210.1.S1_at | B1699931 | Transcription | Transcription | Unclassified | |
| | | | | Factor | | |
| 355 | Gma.14567.1.S1_at | BE824123 | Disease & Defense | | | |
| 356 | Gma.14325.1.S1_at | B1700686 | Disease & Defense | | | Ca2+-independent |
| | | | | | | phospholipase A2 |
| 357 | Gma.4372.1.S1_at | A1443505 | Secondary | | | Reductases with |
| | | | Metabolism | | | broad range of substrate |
| | | | | | | specificities |
| 358 | GmaAffx.66676.1.S1_at | BQ610288 | Unclassified - | | | |
| | | | Protein with | | | |
| | | | Unknown Function | | | |
| 359 | Gma.13418.1.S1_at | BM143894 | Transporter | | | |
| 360 | GmaAffx.32765.1.S1_at | BE024072 | No Homology to | | | |
| | | | Known Proteins | | | |
| 361 | GmaAffx.23276.1.S1_at | AI856198 | Transporter | | | Amino acid |
| | | | | | | transporters |
| 362 | GmaAffx.24359.1.A1_at | BU544840 | Metabolism | | | |
| 363 | GmaAffx.91561.1.S1_s_at | CF807286 | Transporter | | | Apolipoprotein |
| | | | | | | D/Lipocalin |
| 364 | Gma.5084.1.S1_at | AW309848 | Protein | Storage | | |
| | | | Destination | Protein | | |
| | | | & Storage | | | |
| 365 | Gma.9056.1.S1_at | CA819787 | Metabolism | | | Acyl-CoA-binding |
| | | | | | | protein |
| 366 | GmaAffx.70056.1.S1_at | BU765705 | No Homology to | | | |
| | | | Known Proteins | | | |
| 367 | Gma.10612.1.S1_s_at | BG650314 | No Homology to | | | |
| | | | Known Proteins | | | |
| 368 | GmaAffx.1301.52.S1_at | BG510366 | Unclassified - | | | |
| | | | Hypothetical Protein | | | |
| | | | Supported by | | | |
| | | | cDNA | | | |
| 369 | GmaAffx.65217.1.A1_at | BU543988 | Metabolism | | | Acyl-CoA-binding |
| | | | | | | protein |
| 370 | Gma.17729.1.S1_s_at | CD393936 | No Homology to | | | |
| | | | Known Proteins | | | |
| 371 | GmaAffx.83964.1.S1_at | BU082747 | Unclassified - | | | Protein involved |
| | | | Protein with | | | in mRNA turnover |
| | | | Unknown Function | | | and stability |
| 372 | GmaAffx.33408.1.S1_at | BU547044 | No Homology to | | | |
| | | | Known Proteins | | | |
| 373 | GmaAffx.46681.1.S1_at | BQ080794 | Unclassified - | | | |
| | | | Hypothetical Protein | | | |
| | | | Supported by | | | |
| | | | cDNA | | | |
| 374 | Gma.5256.1.A1_at | CD408042 | No Homology to | | | |
| | | | Known Proteins | | | |
| 375 | Gma.11277.1.S1_x_at | CA802310 | Protein | | | |
| | | | Destination | | | |
| | | | & Storage | | | |
| 376 | Gma.2952.1.S1_at | BM270250 | No Homology to | | | |
| | | | Known Proteins | | | |
| 377 | Gma.15863.1.S1_s_at | CD413163 | Metabolism | | | |
| 378 | Gma.5255.1.S1_at | AI736533 | Unclassified - | | | |
| | | | Hypothetical Protein | | | |
| | | | Supported by | | | |
| | | | cDNA | | | |
| 379 | Gma.12902.1.A1_at | CD395017 | Metabolism | | | Hydroxyindole-O- |
| | | | | | | methyltransferase and |
| | | | | | | related SAM-dependent |
| | | | | | | methyltransferases |
| 380 | Gma.3917.1.S1_at | BE658782 | Cell Structure | | | Pectin acetylesterase |
| | | | | | | and similar proteins |
| 381 | Gma.9907.1.S1_at | AW309518 | Transcription | Transcription | Basic | |
| | | | | Factor | Helix- | |
| | | | | | Loop- | |
| | | | | | Helix | |
| | | | | | (bHLH) | |
| 382 | GmaAffx.37930.1.A1_at | BU547709 | Cell Structure | | | |
| 383 | Gma.5578.4.S1_a_at | BE023932 | Metabolism | Cytochrome | CYP98A | Cytochrome P450 |
| | | | | P450 | | CYP2 subfamily |
| 384 | GmaAffx.37458.1.S1_at | BU549419 | Unclassified - | | | |
| | | | Protein with | | | |
| | | | Unknown Function | | | |
| 385 | GmaAffx.64075.1.S1_at | B1700868 | Unclassified - | | | |
| | | | Hypothetical Protein | | | |
| | | | Supported by | | | |
| | | | cDNA | | | |
| 386 | GmaAffx.88945.1.S1_at | CK605738 | Unclassified - | | | |
| | | | Protein with | | | |
| | | | Unknown Function | | | |
| 387 | GmaAffx.92070.1.S1_at | CF807049 | Cell Structure | | | O-methyltransferase |
| 388 | GmaAffx.10908.1.S1_at | BE822295 | Transcription | Transcription | Homeo- | |
| | | | | Factor | domain | |
| 389 | Gma.14361.1.A1_at | CD409843 | No Homology to | | | |
| | | | Known Proteins | | | |
| 390 | GmaAffx.93183.1.S1_s_at | CF808908 | Secondary | | | S-adenosylmethionine |
| | | | Metabolism | | | decarboxylase |
| 391 | GmaAffx.46103.1.S1_at | BM091933 | Metabolism | | | Hydroxymethylglutaryl- |
| | | | | | | CoA lyase |
| 392 | Gma.6782.2.S1_a_at | AW156813 | Unclassified - | | | |
| | | | Hypothetical Protein | | | |
| | | | Supported by | | | |
| | | | cDNA | | | |
| 393 | Gma.8312.1.A1_at | AW310254 | Unclassified - | | | |
| | | | Protein with | | | |
| | | | Unknown Function | | | |
| 394 | GmaAffx.86844.1.S1_at | BE822146 | Unclassified - | | | |
| | | | Protein with | | | |
| | | | Unknown Function | | | |
| 395 | Gma.4093.1.S1_at | BI700897 | Transporter | | | |
| 396 | GmaAffx.17098.1.S1_at | BM084976 | Disease & Defense | | | |
| 397 | GmaAffx.33778.1.S1_at | CA819035 | Protein | Storage | | |
| | | | Destination | Protein | | |
| | | | & Storage | | | |
| 398 | Gma.7107.1.S1_at | A1855972 | Unclassified - | | | |
| | | | Protein with | | | |
| | | | Unknown Function | | | |
| 399 | Gma.4340.2.S1_a_at | BE611760 | Secondary | | | Hydroxyindole-O- |
| | | | Metabolism | | | methyltransferase and |
| | | | | | | related SAM-dependent |
| | | | | | | methyltransferases |
| 400 | Gma.13335.1.S1_at | CD417489 | Unclassified - | | | |
| | | | Hypothetical Protein | | | |
| | | | Supported by | | | |
| | | | cDNA | | | |
| 401 | GmaAffx.2716.1.A1_at | BU547858 | Transcription | Transcription | Zinc | Protein involved |
| | | | | Factor | Finger | in mRNA turnover |
| | | | | | | and stability |
| 402 | Gma.4897.1.S1_at | B1969641 | Unclassified - | | | Thioredoxin, |
| | | | Protein with | | | nucleoredoxin and |
| | | | Unknown Function | | | related proteins |
| 403 | GmaAffx.91254.1.S1_x_at | CF806979 | Unclassified - | | | |
| | | | Hypothetical Protein | | | |
| | | | Supported by | | | |
| | | | cDNA | | | |
| 404 | GmaAffx.84144.1.A1_s_at | CD414061 | No Homology to | | | |
| | | | Known Proteins | | | |
| 405 | GmaAffx.63553.1.S1_at | BG352380 | Energy | | | Cytochrome b |
| 406 | GmaAffx.47819.1.A1_s_at | CD416142 | Unclassified - | | | Predicted |
| | | | Hypothetical Protein | | | mitochondrial |
| | | | Supported by | | | ribosomal protein |
| | | | cDNA | | | L23 |
| 407 | Gma.5097.1.A1_at | B1969945 | Unclassified - | | | |
| | | | Hypothetical Protein | | | |
| | | | Supported by | | | |
| | | | cDNA | | | |
| 408 | Gma.15359.2.S1_a_at | AW781747 | Intracellular | | | Predicted |
| | | | Traffic | | | Ca2+-dependent |
| | | | | | | phospholipid- |
| | | | | | | binding protein |
| 409 | GmaAffx.84566.1.S1_x_at | B1786849 | Transcription | Transcription | MYB | Transcription factor, |
| | | | | Factor | | Myb superfamily |
| 410 | Gma.12211.2.S1_at | BQ299492 | Disease & Defense | | | |
| 411 | Gma.17805.1.A1_s_at | B1968729 | Disease & Defense | | | |
| 412 | Gma.8484.1.S1_at | AF197946.1 | Signal | Protein | | |
| | | | Transduction | Kinase | | |
| 413 | Gma.6213.1.S1_s_at | CD401652 | Protein | | | Glutathione |
| | | | Destination | | | S-transferase |
| | | | & Storage | | | |
| 414 | Gma.2712.1.S1_at | AJ003246.1 | Secondary | | | Flavonol reductase/ |
| | | | Metabolism | | | cinnamoyl-CoA reductase |
| 415 | GmaAffx.89867.1.A1_s_at | CK606556 | Metabolism | | | Predicted PhzC/ |
| | | | | | | PhzF-type epimerase |
| 416 | Gma.2302.1.S1_x_at | AW307490 | Disease & Defense | | | Cysteine proteinase |
| | | | | | | Cathepsin L |
| 417 | GmaAffx.66301.1.S1_at | BG510223 | Secondary | Ethylene | | Iron/ascorbate family |
| | | | Metabolism | Biosynthesis | | oxidoreductases |
| 418 | GmaAffx.1620.3.S1_s_at | AW132226 | No Homology to | | | |
| | | | Known Proteins | | | |
| 419 | Gma.157.1.S1_at | D86351.1 | Metabolism | Cytochrome | CYP93D | Cytochrome P450 |
| | | | | P450 | | CYP2 subfamily |
| 420 | GmaAffx.9127.1.S1_s_at | B1317582 | Transcription | Transcription | Zinc | FOG: Predicted E3 |
| | | | | Factor | Finger | ubiquitin ligase |
| 421 | GmaAffx.3706.1.S1_at | BE821261 | Unclassified - | | | HGG motif-containing |
| | | | Hypothetical Protein | | | thioesterase |
| | | | NOT Supported by | | | |
| | | | cDNA | | | |
| 422 | GmaAffx.82564.1.S1_s_at | BG047418 | No Homology to | | | |
| | | | Known Proteins | | | |
| 423 | Gma.9622.1.S1_x_at | BQ610840 | Metabolism | | | Hydroxyindole-O- |
| | | | | | | methyltransferase and |
| | | | | | | related SAM-dependent |
| | | | | | | methyltransferases |
| 424 | Gma.3581.2.S1_x_at | CD398783 | Unclassified - | | | Ca2+-dependent |
| | | | Hypothetical Protein | | | lipid-binding protein |
| | | | NOT Supported by | | | CLB1/vesicle protein |
| | | | cDNA | | | vp115/Granuphilin |
| | | | | | | A, contains C2 domain |
| 425 | GmaAffx.75594.1.S1_at | AW307139 | Secondary | | | Multicopper oxidases |
| | | | Metabolism | | | |
| 426 | GmaAffx.87101.1.S1_at | BM309451 | No Homology to | | | |
| | | | Known Proteins | | | |
| 427 | GmaAffx.55362.1.S1_at | BE475283 | Unclassified - | | | Reticulon |
| | | | Hypothetical Protein | | | |
| | | | Supported by | | | |
| | | | cDNA | | | |
| 428 | Gma.79.4.S1_s_at | AI443819 | Unclassified - | | | |
| | | | Hypothetical Protein | | | |
| | | | Supported by | | | |
| | | | cDNA | | | |
| 429 | Gma.8556.1.S1_at | BU764573 | Transporter | | | Amino acid |
| | | | | | | transporters |
| 430 | GmaAffx.82195.1.S1_at | BM528489 | Unclassified - | | | |
| | | | Hypothetical Protein | | | |
| | | | NOT Supported by | | | |
| | | | cDNA | | | |
| 431 | GmaAffx.90515.1.S1_s_at | CF806240 | Unclassified - | | | Predicted pyroglutamyl |
| | | | Hypothetical Protein | | | peptidase |
| | | | Supported by | | | |
| | | | cDNA | | | |
| 432 | Gma.4578.1.S1_s_at | BE802809 | Transporter | | | Plasma membrane |
| | | | | | | H+-transporting |
| | | | | | | ATPase |
| 433 | GmaAffx.48438.1.S1_at | AW350858 | Protein | | | Molecular chaperone |
| | | | Destination | | | (DnaJ superfamily) |
| | | | & Storage | | | |
| 434 | Gma.15359.1.S1_x_at | BM271008 | Unclassified - | | | Predicted |
| | | | Protein with | | | Ca2+-dependent |
| | | | Unknown Function | | | phospholipid- |
| | | | | | | binding protein |
| 435 | GmaAffx.60672.1.S1_at | BU080558 | No Homology to | | | |
| | | | Known Proteins | | | |
| 436 | Gma.2302.1.S1_at | AW307490 | Disease & Defense | | | Cysteine proteinase |
| | | | | | | Cathepsin L |
| 437 | Gma.13187.1.S1_at | BG839239 | Unclassified - | | | Uncharacterized |
| | | | Hypothetical Protein | | | conserved protein |
| | | | Supported by | | | |
| | | | cDNA | | | |
| 438 | GmaAffx.76696.1.A1_at | BE824386 | No Homology to | | | |
| | | | Known Proteins | | | |
| 439 | GmaAffx.89060.1.S1_at | CK605664 | Metabolism | Cytochrome | CYP93D | Cytochrome P450 |
| | | | | P450 | | CYP2 subfamily |
| 440 | GmaAffx.92112.1.S1_s_at | CF807837 | No Homology to | | | |
| | | | Known Proteins | | | |
| 441 | GmaAffx.93157.1.S1_s_at | CF808882 | Metabolism | | | Aldo/keto reductase |
| | | | | | | family proteins |
| 442 | Gma.4305.1.S1_at | AW350687 | Disease & Defense | | | |
| 443 | GmaAffx.91037.1.S1_s_at | CF806762 | Secondary | | | Methyltransferase |
| | | | Metabolism | | | |
| 444 | GmaAffx.74300.1.S1_at | BU080963 | Disease & Defense | | | |
| 445 | GmaAffx.18113.2.S1_s_at | AW706795 | Unclassified - | Hormone | | |
| | | | Protein with | Induced | | |
| | | | Unknown Function | | | |
| 446 | Gma.2718.1.S1_at | CD406302 | Transporter | | | Multidrug |
| | | | | | | resistance-associated |
| | | | | | | protein/mitoxantrone |
| | | | | | | resistance protein, |
| | | | | | | ABC superfamily |
| 447 | Gma.9236.1.S1_at | BG405642 | Metabolism | Amino Acid | | Spermidine synthase |
| | | | | Biosynthesis/ | | |
| | | | | Metabolism | | |
| 448 | Gma.5733.1.S1_at | CD402671 | Signal | Calcium | | Ca2+-binding |
| | | | Transduction | Binding | | protein (centrin/ |
| | | | | | | caltractin), EF-Hand |
| | | | | | | superfamily protein |
| 449 | GmaAffx.90703.1.A1_at | CF809087 | Disease & Defense | | | |
| 450 | GmaAffx.79390.2.S1_at | AW597383 | Transcription | Transcription | Basic | FOG: Armadillo/beta- |
| | | | | Factor | Leucine | catenin-like repeats |
| | | | | | Zipper | |
| | | | | | (bZIP) | |
| 451 | GmaAffx.31838.1.S1_at | BU547835 | Secondary | Flavonoid | | Flavonol reductase/ |
| | | | Metabolism | Biosynthesis | | cinnamoyl-CoA |
| | | | | | | reductase |
| 452 | Gma.8394.1.S1_at | CD392332 | Disease & Defense | | | Prohibitins and |
| | | | | | | stomatins of the PID |
| | | | | | | superfamily |
| 453 | GmaAffx.727.1.S1_at | BE804565 | Unclassified - | | | Ubiquinone |
| | | | Protein with | | | biosynthesis |
| | | | Unknown Function | | | methyltransferase |
| | | | | | | COQ5 |
| 454 | Gma.5578.1.S1_x_at | BE659192 | Metabolism | Cytochrome | CYP98A | Cytochrome P450 |
| | | | | P450 | | CYP2 subfamily |
| 455 | GmaAffx.93607.1.S1_s_at | CF809332 | Unclassified - | | | |
| | | | Protein with | | | |
| | | | Unknown Function | | | |
| 456 | Gma.11277.2.S1_a_at | B1701238 | Unclassified - | | | |
| | | | Hypothetical Protein | | | |
| | | | NOT Supported by | | | |
| | | | cDNA | | | |
| 457 | GmaAffx.71711.1.S1_at | AI988272 | No Homology to | | | |
| | | | Known Proteins | | | |
| 458 | Gma.3345.1.S1_at | AW394524 | Metabolism | | | Alcohol dehydrogenase, |
| | | | | | | class III |
| 459 | GmaAffx.90069.1.S1_at | CF805794 | Unclassified - | | | Leucine rich repeat |
| | | | Hypothetical Protein | | | proteins, some |
| | | | Supported by | | | proteins contain |
| | | | cDNA | | | F-box |
| 460 | Gma.6187.1.S1_at | BF324343 | No Homology to | | | |
| | | | Known Proteins | | | |
| 461 | Gma.12211.1.S1_at | AW394548 | Disease & Defense | | | |
| 462 | Gma.3880.1.S1_at | BE659305 | Unclassified - | | | |
| | | | Hypothetical Protein | | | |
| | | | NOT Supported by | | | |
| | | | cDNA | | | |
| 463 | GmaAffx.32328.1.A1_at | BI968685 | No Homology to | | | |
| | | | Known Proteins | | | |
| 464 | GmaAffx.56606.1.S1_at | BG507644 | Secondary | | | Multicopper oxidases |
| | | | Metabolism | | | |
| 465 | Gma.2590.1.A1_s_at | CD406617 | Disease & Defense | | | |
| 466 | GmaAffx.90652.1.S1_at | CF806377 | Unclassified - | | | Predicted hydrolase/ |
| | | | Hypothetical Protein | | | acyltransferase |
| | | | Supported by | | | (alpha/beta hydrolase |
| | | | cDNA | | | superfamily) |
| 467 | GmaAffx.80830.1.A1_at | BU549591 | Unclassified - | | | |
| | | | Hypothetical Protein | | | |
| | | | Supported by | | | |
| | | | cDNA | | | |
| 468 | Gma.6763.1.A1_at | BU544794 | Energy | | | Ferric reductase, |
| | | | | | | NADH/NADPH oxidase |
| | | | | | | and related proteins |
| 469 | Gma.6623.2.S1_s_at | BQ629284 | Unclassified - | | | 1,4-benzoquinone |
| | | | Protein with | | | reductase-like; Trp |
| | | | Unknown Function | | | repressor binding |
| | | | | | | protein-like/ |
| | | | | | | protoplast-secreted |
| | | | | | | protein |
| 470 | GmaAffx.92568.1.S1_at | CF808293 | Metabolism | | | Acyl-CoA-binding |
| | | | | | | protein |
| 471 | Gma.9534.1.S1_at | CA802874 | Disease & Defense | | | |
| 472 | Gma.11284.1.S1_at | AW153035 | Disease & Defense | | | |
| 473 | Gma.6550.1.A1_at | CD402638 | Metabolism | | | Hydroxyindole-O- |
| | | | | | | methyltransferase and |
| | | | | | | related SAM-dependent |
| | | | | | | methyltransferases |
| 474 | Gma.426.1.A1_at | A1442768 | Transcription | Transcription | Zinc | |
| | | | | Factor | Finger | |
| 475 | GmaAffx.45805.2.S1_at | CA784263 | Metabolism | | | Hydroxyindole-O- |
| | | | | | | methyltransferase and |
| | | | | | | related SAM-dependent |
| | | | | | | methyltransferases |
| 476 | GmaAffx.84566.1.S1_at | B1786849 | Transcription | Transcription | MYB | Transcription factor, |
| | | | | Factor | | Myb superfamily |
| 477 | Gma.14897.2.S1_at | BQ785154 | Unclassified - | | | Dihydroorotase and |
| | | | Protein with | | | related enzymes |
| | | | Unknown Function | | | |
| 478 | Gma.8515.1.S1_at | AF243372.1 | Protein | | | Glutathione S- |
| | | | Destination | | | transferase |
| | | | & Storage | | | |
| 479 | GmaAffx.53914.1.A1_at | BE822062 | No Homology to | | | |
| | | | Known Proteins | | | |
| 480 | Gma.4470.1.S1_s_at | AW156251 | Unclassified - | | | |
| | | | Protein with | | | |
| | | | Unknown Function | | | |
| 481 | Gma.15359.1.S1_a_at | BM271008 | Unclassified - | | | Predicted |
| | | | Protein with | | | Ca2+-dependent |
| | | | Unknown Function | | | phospholipid-binding |
| | | | | | | protein |
| 482 | Gma.5578.3.S1_a_at | AW310572 | Metabolism | Cytochrome | CYP98A | Cytochrome P450 CYP2 |
| | | | | P450 | | subfamily |
| 483 | GmaAffx.76821.1.S1_at | BG238158 | Unclassified - | Hormone | | |
| | | | Protein with | Induced | | |
| | | | Unknown Function | | | |
| 484 | GmaAffx.91328.1.S1_s_at | CF807053 | Unclassified - | | | Leucine rich |
| | | | Hypothetical Protein | | | repeat proteins, some |
| | | | Supported by | | | proteins contain |
| | | | cDNA | | | F-box |
| 485 | GmaAffx.40015.1.S1_at | BE609673 | No Homology to | | | |
| | | | Known Proteins | | | |
| 486 | Gma.7102.1.S1_a_at | AW309275 | Unclassified - | | | Leucine rich |
| | | | Hypothetical Protein | | | repeat proteins, some |
| | | | Supported by | | | proteins contain |
| | | | cDNA | | | F-box |
| 487 | GmaAffx.20108.1.S1_at | CA801220 | Unclassified - | | | |
| | | | Hypothetical Protein | | | |
| | | | Supported by | | | |
| | | | cDNA | | | |
| 488 | GmaAffx.92148.1.S1_s_at | CF807873 | Unclassified - | | | |
| | | | Hypothetical Protein | | | |
| | | | NOT Supported by | | | |
| | | | cDNA | | | |
| 489 | GmaAffx.37803.2.S1_at | BE347017 | No Homology to | | | |
| | | | Known Proteins | | | |
| 490 | GmaAffx.51466.1.A1_at | CD406245 | Metabolism | | | UDP-glucuronosyl |
| | | | | | | and UDP-glucosyl |
| | | | | | | transferase |
| 491 | Gma.2102.1.S1_s_at | BM523392 | Disease & Defense | | | |
| 492 | GmaAffx.83172.1.S1_at | BQ610576 | Unclassified - | | | |
| | | | Protein with | | | |
| | | | Unknown Function | | | |
| 493 | Gma.6730.1.S1_s_at | BE211440 | Metabolism | | | UDP-glucuronosyl |
| | | | | | | and UDP-glucosyl |
| | | | | | | transferase |
| 494 | GmaAffx.85662.1.S1_at | BG404902 | Unclassified - | | | Predicted membrane |
| | | | Protein with | | | protein, contains |
| | | | Unknown Function | | | DoH and Cytochrome |
| | | | | | | b-561/ferric reductase |
| | | | | | | transmembrane domains |
| 495 | Gma.7836.1.A1_at | BU549650 | Unclassified - | | | |
| | | | Hypothetical Protein | | | |
| | | | NOT Supported by | | | |
| | | | cDNA | | | |
| 496 | Gma.11277.1.S1_a_at | CA802310 | Protein | | | |
| | | | Destination | | | |
| | | | & Storage | | | |
| 497 | Gma.9086.2.S1_at | BE555169 | Cell Growth | | | |
| | | | & Division | | | |
| 498 | GmaAffx.50811.2.S1_at | BG725712 | Transcription | Transcription | NAC | |
| | | | | Factor | Domain | |
| 499 | GmaAffx.72630.1.S1_at | BG239729 | Unclassified - | | | FOG: TPR repeat |
| | | | Hypothetical Protein | | | |
| | | | NOT Supported by | | | |
| | | | cDNA | | | |
| 499 | GmaAffx.72630.1.S1_at | BG239729 | Unclassified - | | | Thioredoxin |
| | | | Hypothetical Protein | | | |
| | | | NOT Supported by | | | |
| | | | cDNA | | | |
| 500 | Gma.9856.1.S1_at | BG650352 | Signal Transduction | Protein | | Serine/threonine |
| | | | | Kinase | | protein kinase |
| 501 | GmaAffx.5743.1.S1_at | BQ628343 | Unclassified - | | | FOG: Predicted E3 |
| | | | Hypothetical Protein | | | ubiquitin ligase |
| | | | Supported by | | | |
| | | | cDNA | | | |
| 502 | Gma.358.1.S1_at | AW310270 | Metabolism | | | Arylacetamide |
| | | | | | | deacetylase |
| 503 | Gma.2439.1.S1_at | B1972053 | Metabolism | | | Predicted PhzC/ |
| | | | | | | PhzF-type epimerase |
| 504 | GmaAffx.30421.1.S1_at | BU082092 | Transcription | Transcription | Zinc | FOG: Predicted E3 |
| | | | | Factor | Finger | ubiquitin ligase |
| 505 | GmaAffx.22552.1.S1_at | BU549952 | Transporter | | | H+/oligopeptide |
| | | | | | | symporter |
| 506 | GmaAffx.93301.2.S1_s_at | CF808497 | Secondary | | | Flavonol reductase/ |
| | | | Metabolism | | | cinnamoyl-CoA reductase |
| 507 | Gma.6280.2.S1_at | A1938985 | No Homology to | | | |
| | | | Known Proteins | | | |
| 508 | Gma.418.1.A1_at | CA784607 | Unclassified - | | | Glycosyl transferase, |
| | | | Hypothetical Protein | | | family 8 - glycogenin |
| | | | Supported by | | | |
| | | | cDNA | | | |
| 509 | Gma.13910.1.S1_at | CD409745 | Unclassified - | | | |
| | | | Protein with | | | |
| | | | Unknown Function | | | |
| 510 | GmaAffx.89508.1.A1_s_at | CK606172 | Secondary | | | Phenylalanine and |
| | | | Metabolism | | | histidine ammonia- |
| | | | | | | lyase |
| 511 | Gma.10804.2.A1_s_at | CF921045 | Protein | | | Molecular chaperone |
| | | | Destination | | | (DnaJ superfamily) |
| | | | & Storage | | | |
| 512 | GmaAffx.52337.1.A1_at | CD413673 | No Homology to | | | |
| | | | Known Proteins | | | |
| 513 | Gma.8988.1.S1_at | BU965547 | No Homology to | | | |
| | | | Known Proteins | | | |
| 514 | GmaAffx.52391.1.S1_at | AW349563 | Unclassified - | | | |
| | | | Hypothetical Protein | | | |
| | | | NOT Supported by | | | |
| | | | cDNA | | | |
| 515 | Gma.13856.1.A1_at | CD408222 | No Homology to | | | |
| | | | Known Proteins | | | |
| 516 | GmaAffx.1242.1.A1_at | BU549839 | Metabolism | | | |
| 517 | Gma.15184.1.S1_at | BM522992 | Transcription | Transcription | Homeo- | Transcription factor |
| | | | | Factor | domain | HEX, contains HOX |
| | | | | | | and HALZ domains |
| 518 | GmaAffx.73554.1.S1_at | BM139469 | Unclassified - | | | |
| | | | Hypothetical Protein | | | |
| | | | NOT Supported by | | | |
| | | | cDNA | | | |
| 519 | GmaAffx.54085.2.S1_at | BQ629483 | No Homology to | | | |
| | | | Known Proteins | | | |
| 520 | GmaAffx.88017.1.A1_at | BQ452554 | No Homology to | | | |
| | | | Known Proteins | | | |
| 521 | Gma.8441.1.S1_at | AY113187.2 | Secondary | | | Multicopper oxidases |
| | | | Metabolism | | | |
| 522 | Gma.6642.1.A1_at | BE610052 | Transcription | Transcription | Basic | FOG: Armadillo/beta- |
| | | | | Factor | Leucine | catenin-like repeats |
| | | | | | Zipper | |
| | | | | | (bZIP) | |
| 523 | GmaAffx.73943.1.S1_at | BM891115 | Unclassified - | | | |
| | | | Hypothetical Protein | | | |
| | | | Supported by | | | |
| | | | cDNA | | | |
| 524 | Gma.3969.1.S1_s_at | AW349494 | Energy | | | |
| 525 | GmaAffx.52114.1.S1_at | CA852143 | Metabolism | | | Heme oxygenase |
| 526 | GmaAffx.34453.1.S1_at | BU544114 | Unclassified - | | | |
| | | | Hypothetical Protein | | | |
| | | | Supported by | | | |
| | | | cDNA | | | |
| 527 | GmaAffx.53256.1.S1_at | BU545086 | Unclassified - | | | |
| | | | Hypothetical Protein | | | |
| | | | Supported by | | | |
| | | | cDNA | | | |
| 528 | Gma.57.1.S1_at | AF532624.1 | Unclassified - | | | |
| | | | Hypothetical Protein | | | |
| | | | NOT Supported by | | | |
| | | | cDNA | | | |
| 529 | Gma.5711.1.S1_at | B1784811 | Unclassified - | | | |
| | | | Hypothetical Protein | | | |
| | | | Supported by | | | |
| | | | cDNA | | | |
| 530 | GmaAffx.42587.1.A1_at | BU544395 | No Homology to | | | |
| | | | Known Proteins | | | |
| 531 | GmaAffx.43297.1.A1_at | A1442864 | Unclassified - | | | |
| | | | Hypothetical Protein | | | |
| | | | NOT Supported by | | | |
| | | | cDNA | | | |
| 532 | GmaAffx.35656.1.A1_at | BE822348 | No Homology to | | | |
| | | | Known Proteins | | | |
| 533 | Gma.4746.1.A1_s_at | CD404836 | No Homology to | | | |
| | | | Known Proteins | | | |
| 534 | Gma.149.1.S1_at | U43839.1 | Metabolism | | | Choline kinase |
| 535 | GmaAffx.48718.1.S1_at | BM528538 | No Homology to | | | |
| | | | Known Proteins | | | |
| 536 | Gma.11522.2.S1_at | BG650872 | Unclassified - | | | |
| | | | Hypothetical Protein | | | |
| | | | Supported by | | | |
| | | | cDNA | | | |
| 537 | GmaAffx.92649.1.S1_at | CF808374 | Metabolism | | | |
| 538 | Gma.8966.1.S1_at | BU765374 | Protein | | | Predicted lipase |
| | | | Destination | | | |
| | | | & Storage | | | |
| 539 | GmaAffx.92693.1.S1_at | CF808418 | No Homology to | | | |
| | | | Known Proteins | | | |
| 540 | GmaAffx.27496.2.S1_at | BM523609 | Metabolism | | | |
| 541 | Gma.10312.1.S1_at | AW706614 | Unclassified - | | | |
| | | | Hypothetical Protein | | | |
| | | | NOT Supported by | | | |
| | | | cDNA | | | |
| 542 | GmaAffx.81750.2.S1_at | BM568090 | Transcription | Transcription | Homeo- | Transcription factor |
| | | | | Factor | domain | MEIS1 and related |
| | | | | | | HOX domain proteins |
| 543 | GmaAffx.43809.1.S1_at | AW396565 | No Homology to | | | |
| | | | Known Proteins | | | |
| 544 | Gma.3951.1.S1_at | BE659337 | Unclassified - | | | |
| | | | Hypothetical Protein | | | |
| | | | NOT Supported by | | | |
| | | | cDNA | | | |
| 545 | GmaAffx.14279.1.S1_at | BM887020 | Metabolism | | | |
| 546 | Gma.6481.1.S1_at | CD416703 | Unclassified - | | | |
| | | | Hypothetical Protein | | | |
| | | | Supported by | | | |
| | | | cDNA | | | |
| 547 | Gma.9957.1.S1_at | BE820632 | Metabolism | | | UDP-glucuronosyl |
| | | | | | | and UDP-glucosyl |
| | | | | | | transferase |
| 548 | GmaAffx.45036.1.S1_at | BF595470 | No Homology to | | | |
| | | | Known Proteins | | | |
| 549 | Gma.2801.1.S1_at | B1785739 | Cell Structure | | | Beta-glucosidase, |
| | | | | | | lactase phlorizinhydrolase, |
| | | | | | | and related proteins |
| 550 | GmaAffx.93436.1.A1_s_at | CF809161 | Transcription | Transcription | AP2/ | |
| | | | | Factor | EREBP | |
| 551 | Gma.8704.1.S1_at | AW311022 | Unclassified - | | | FOG: TPR repeat |
| | | | Hypothetical Protein | | | |
| | | | NOT Supported by | | | |
| | | | cDNA | | | |
| 551 | Gma.8704.1.S1_at | AW311022 | Unclassified - | | | Thioredoxin |
| | | | Hypothetical Protein | | | |
| | | | NOT Supported by | | | |
| | | | cDNA | | | |
| 552 | GmaAffx.90677.1.S1_at | CF806402 | Unclassified - | | | DNA helicase |
| | | | Hypothetical Protein | | | PIF1/RRM3 |
| | | | NOT Supported by | | | |
| | | | cDNA | | | |
| 553 | Gma.4120.2.S1_at | BI700246 | Unclassified - | | | |
| | | | Protein with | | | |
| | | | Unknown Function | | | |
| 554 | Gma.9036.1.S1_at | AW309501 | No Homology to | | | |
| | | | Known Proteins | | | |
| 555 | GmaAffx.55959.1.S1_at | BQ629429 | Unclassified - | | | |
| | | | Hypothetical Protein | | | |
| | | | NOT Supported by | | | |
| | | | cDNA | | | |
| 556 | Gma.3575.1.S1_at | BQ611599 | Cell Structure | | | |
| 557 | Gma.5961.1.S1_at | AB052786.1 | Transporter | | | H+/oligopeptide |
| | | | | | | symporter |
| 558 | GmaAffx.39765.1.S1_s_at | BQ785586 | No Homology to | | | |
| | | | Known Proteins | | | |
| 559 | Gma.4479.1.S1_at | CA936529 | Unclassified - | | | |
| | | | Hypothetical Protein | | | |
| | | | Supported by | | | |
| | | | cDNA | | | |
| 560 | Gma.10969.2.S1_at | CD402329 | Unclassified - | | | Predicted lipase |
| | | | Hypothetical Protein | | | |
| | | | NOT Supported by | | | |
| | | | cDNA | | | |
| 561 | GmaAffx.77758.1.S1_x_at | BI944003 | No Homology to | | | |
| | | | Known Proteins | | | |
| 562 | Gma.10621.1.S1_at | AY004243.1 | Unclassified - | | | |
| | | | Hypothetical Protein | | | |
| | | | Supported by | | | |
| | | | cDNA | | | |
| 563 | Gma.14606.1.A1_at | BE821928 | Transporter | | | |
| 564 | GmaAffx.24992.1.S1_at | BU083490 | Protein | | | Aspartyl protease |
| | | | Destination | | | |
| | | | & Storage | | | |
| 565 | GmaAffx.72700.1.S1_at | AW349825 | Unclassified - | | | |
| | | | Hypothetical Protein | | | |
| | | | NOT Supported by | | | |
| | | | cDNA | | | |
| 566 | Gma.11026.1.S1_at | AW756869 | Disease & Defense | | | |
| 567 | GmaAffx.14279.2.A1_at | BM095037 | Metabolism | | | |
| 568 | Gma.3799.2.S1_s_at | AW348702 | Unclassified - | | | |
| | | | Protein with | | | |
| | | | Unknown Function | | | |
| 569 | Gma.5863.1.S1_at | AJ003245.1 | Secondary | | | |
| | | | Metabolism | | | |
| 570 | GmaAffx.47806.1.A1_at | BG508458 | No Homology to | | | |
| | | | Known Proteins | | | |
| 571 | GmaAffx.90677.1.S1_s_at | CF806402 | Unclassified - | | | DNA helicase |
| | | | Hypothetical Protein | | | PIF1/RRM3 |
| | | | NOT Supported by | | | |
| | | | cDNA | | | |
| 572 | Gma.4928.1.S1_a_at | AF002259.1 | Disease & Defense | | | Acyl-CoA synthetase |
| 573 | GmaAffx.93575.1.S1_s_at | CF808812 | No Homology to | | | |
| | | | Known Proteins | | | |
| 574 | Gma.4266.1.S1_at | BI972260 | No Homology to | | | |
| | | | Known Proteins | | | |
| 575 | Gma.10827.3.S1_at | AW758966 | Secondary | | | Iron/ascorbate family |
| | | | Metabolism | | | oxidoreductases |
| 576 | GmaAffx.76276.1.S1_at | BE331452 | Transporter | | | Copper chaperone |
| 577 | GmaAffx.26516.1.S1_at | AW433266 | Cell Structure | | | |
| 578 | Gma.5716.1.S1_at | BQ453925 | No Homology to | | | |
| | | | Known Proteins | | | |
| 579 | GmaAffx.89567.1.A1_at | CK606157 | Signal | Calcium | Calcium | Ca2+/calmodulin- |
| | | | Transduction | Binding | Dependent | dependent protein |
| | | | | | Protein | kinase, EF-Hand |
| | | | | | Kinase | protein superfamily |
| 580 | GmaAffx.89396.2.A1_s_at | CK605844 | Cell Structure | | | |
| 581 | GmaAffx.2743.1.S1_at | BM568087 | Signal | Protein | | |
| | | | Transduction | Kinase | | |
| 582 | GmaAffx.70509.1.A1_at | CD400633 | No Homology to | | | |
| | | | Known Proteins | | | |
| 583 | GmaAffx.69888.1.A1_at | BU549275 | No Homology to | | | |
| | | | Known Proteins | | | |
| 584 | GmaAffx.87872.1.S1_at | BM885036 | Metabolism | | | CTP synthase |
| | | | | | | (UTP-ammonia lyase) |
| 585 | GmaAffx.18846.1.S1_at | BM091991 | Signal | Small | | FOG: WD40 repeat |
| | | | Transduction | GTPase | | |
| 586 | GmaAffx.89158.1.A1_at | CK605958 | Intracellular | | | Phosphatidylinositol |
| | | | Traffic | | | transfer protein SECI4 |
| | | | | | | and related proteins |
| 587 | GmaAffx.52494.1.S1_at | BE473800 | Unclassified - | | | |
| | | | Hypothetical Protein | | | |
| | | | NOT Supported by | | | |
| | | | cDNA | | | |
| 588 | GmaAffx.48172.1.S1_at | BF009291 | Transporter | | | Predicted small molecule |
| | | | | | | transporter |
| 589 | Gma.7726.1.A1_at | BM886413 | Unclassified - | | | Ca2+-binding protein |
| | | | Protein with | | | (centrin/caltractin), |
| | | | Unknown Function | | | EF-Hand superfamily protein |
| 590 | Gma.15889.1.S1_at | AW348133 | Transcription | Transcription | WRKY | |
| | | | | Factor | | |
| 591 | Gma.2010.1.S1_a_at | CD402050 | Transcription | Transcription | Zinc | GATA-4/5/6 transcription |
| | | | | Factor | Finger | factors |
| 592 | GmaAffx.55767.1.S1_at | BQ079515 | Signal Transduction | Calcium | | Calmodulin and related proteins |
| | | | | Binding | | (EF-Hand superfamily) |
| 593 | GmaAffx.78720.2.S1_at | BI788325 | No Homology to | | | |
| | | | Known Proteins | | | |
| 594 | PsAffx.psHB044xM02f_at | | | | | |
| 595 | Gma.5631.1.S1_at | CD405214 | Transporter | | | Amino acid transporters |
| 596 | GmaAffx.24583.1.S1_at | BE609540 | Signal | Protein | | Serine/threonine protein |
| | | | Transduction | Kinase | | kinase |
| 597 | Gma.2446.2.S1_at | B1427101 | Unclassified - | | | |
| | | | Hypothetical Protein | | | |
| | | | Supported by | | | |
| | | | cDNA | | | |
| 598 | GmaAffx.93270.1.S1_x_at | CF808995 | No Homology to | | | |
| | | | Known Proteins | | | |
| 599 | GmaAffx.18649.1.S1_at | AW351025 | No Homology to | | | |
| | | | Known Proteins | | | |
| 600 | Gma.12665.2.A1_at | CD394142 | Unclassified - | | | |
| | | | Hypothetical Protein | | | |
| | | | Supported by | | | |
| | | | cDNA | | | |
| 601 | Gma.15889.1.S1_s_at | AW348133 | Transcription | Transcription | WRKY | |
| | | | | Factor | | |
| 602 | Gma.5579.1.S1_at | AW119360 | Secondary | Flavonoid | | |
| | | | Metabolism | Biosynthesis | | |
| 603 | GmaAffx.36514.1.S1_at | BE658341 | Disease & Defense | | | |
| 604 | GmaAffx.47815.1.A1_at | CD415065 | Unclassified - | | | |
| | | | Hypothetical Protein | | | |
| | | | NOT Supported by | | | |
| | | | cDNA | | | |
| 605 | Gma.12665.1.S1_at | BE347113 | Unclassified - | | | |
| | | | Hypothetical Protein | | | |
| | | | Supported by | | | |
| | | | cDNA | | | |
| 606 | GmaAffx.37908.1.A1_at | BU546116 | Signal | Protein | | |
| | | | Transduction | Kinase | | |
| 607 | Gma.12867.1.A1_at | CD393518 | Unclassified - | | | |
| | | | Hypothetical Protein | | | |
| | | | Supported by | | | |
| | | | cDNA | | | |
| 608 | GmaAffx.78434.1.S1_at | BG041904 | Unclassified - | | | Predicted membrane |
| | | | Hypothetical Protein | | | protein, contains |
| | | | NOT Supported by | | | DoH and Cytochrome |
| | | | cDNA | | | b-561/ferric reductase |
| | | | | | | transmembrane domains |
| 609 | Gma.13433.1.A1_at | CD398049 | No Homology to | | | |
| | | | Known Proteins | | | |
| 610 | Gma.5629.2.S1_a_at | BG042866 | Disease & Defense | | | |
| 611 | Gma.2972.1.S1_at | AW350602 | Unclassified - | | | Phosphatidylinositol |
| | | | Hypothetical Protein | | | transfer protein SEC14 |
| | | | Supported by | | | and related proteins |
| | | | cDNA | | | |
| 612 | GmaAffx.56563.1.S1_at | CA820002 | No Homology to | | | |
| | | | Known Proteins | | | |
| 613 | GmaAffx.45530.1.S1_at | BG789917 | Signal | Protein | | |
| | | | Transduction | Kinase | | |
| 614 | GmaAffx.40860.1.S1_at | BQ628787 | Disease & Defense | | | |
| 615 | Gma.8797.1.A1_at | B1969881 | Unclassified - | | | |
| | | | Hypothetical Protein | | | |
| | | | NOT Supported by | | | |
| | | | cDNA | | | |
| 616 | GmaAffx.66979.1.S1_at | B1497616 | Signal | Protein | AtC1PKs | Serine/threonine protein |
| | | | Transduction | Kinase | | kinase |
| 617 | GmaAffx.78720.1.S1_at | BM526650 | No Homology to | | | |
| | | | Known Proteins | | | |
| 618 | Gma.15495.1.A1_at | CD409942 | Unclassified - | | | Serine/threonine protein |
| | | | Hypothetical Protein | | | kinase |
| | | | NOT Supported by | | | |
| | | | cDNA | | | |
| 619 | Gma.6302.1.S1_at | BI786195 | Cell Structure | | | Beta-glucosidase, lactase |
| | | | | | | phlorizinhydrolase, and |
| | | | | | | related proteins |
| 620 | GmaAffx.14179.1.S1_at | BM085107 | Unclassified - | | | SETA binding protein |
| | | | Hypothetical Protein | | | SB1 and related proteins, |
| | | | NOT Supported by | | | contain BTB/POZ domain |
| | | | cDNA | | | |
| 621 | Gma.3988.1.S1_at | BE658819 | Unclassified - | | | |
| | | | Hypothetical Protein | | | |
| | | | Supported by | | | |
| | | | cDNA | | | |
| 622 | GmaAffx.17811.1.S1_at | B1426517 | Transporter | | | Predicted transporter |
| | | | | | | (major facilitator |
| | | | | | | superfamily) |
| 623 | Gma.13027.1.A1_at | CD397394 | Transporter | | | Amino acid transporters |
| 624 | Gma.9913.2.S1_a_at | CD390580 | Transcription | Transcription | WRKY | |
| | | | | Factor | | |
| 625 | Gma.8459.1.S1_at | AF249317.1 | Signal Transduction | Protein | | Serine/threonine |
| | | | | Kinase | | protein kinase |
| 626 | Gma.4832.1.A1_at | AW310458 | Signal Transduction | Protein | | |
| | | | | Kinase | | |
| 627 | Gma.17576.1.S1_s_at | BG650176 | No Homology to | | | |
| | | | Known Proteins | | | |
| 628 | GmaAffx.60595.1.S1_at | BQ628072 | No Homology to | | | |
| | | | Known Proteins | | | |
| 629 | GmaAffx.52050.1.S1_at | BG237350 | Transcription | Transcription | MYB | Transcription factor, |
| | | | | Factor | | Myb superfamily |
| 630 | GmaAffx.88037.2.S1_at | CA783510 | Unclassified - | | | |
| | | | Hypothetical Protein | | | |
| | | | Supported by | | | |
| | | | cDNA | | | |
| 631 | Gma.17605.3.S1_at | BQ079859 | Secondary | Flavonoid | | |
| | | | Metabolism | Biosynthesis | | |
| 632 | GmaAffx.89999.1.S1_at | CF805724 | Disease & Defense | | | |
| 633 | Gma.68.1.S1_at | AF357211.1 | Transcription | Transcription | AP2/ | |
| | | | | Factor | EREBP | |
| 634 | Gma.7921.1.S1_at | BM177567 | Unclassified - | | | Uncharacterized |
| | | | Hypothetical Protein | | | conserved protein |
| | | | Supported by | | | |
| | | | cDNA | | | |
| 635 | GmaAffx.28927.1.S1_at | BU549173 | Transcription | Transcription | MYB | Transcription factor, |
| | | | | Factor | | Myb superfamily |
| 636 | Gma.4169.1.S1_at | BQ473559 | Transporter | | | Copper chaperone |
| 637 | GmaAffx.90946.1.S1_at | CF808349 | Unclassified - | | | |
| | | | Hypothetical Protein | | | |
| | | | NOT Supported by | | | |
| | | | cDNA | | | |
| 638 | GmaAffx.77809.1.A1_at | BI969302 | Disease & Defense | | | |
| 639 | GmaAffx.7215.1.A1_at | AW351149 | Unclassified - | | | |
| | | | Hypothetical Protein | | | |
| | | | NOT Supported by | | | |
| | | | cDNA | | | |
| 640 | GmaAffx.11658.1.S1_at | AW184817 | Signal Transduction | Unclassified | | |
| 641 | Gma.6529.2.S1_s_at | BM309390 | Unclassified - | | | |
| | | | Hypothetical Protein | | | |
| | | | NOT Supported by | | | |
| | | | cDNA | | | |
| 642 | GmaAffx.45241.1.S1_at | BG157181 | Unclassified - | | | Glutaredoxin and |
| | | | Hypothetical Protein | | | related proteins |
| | | | Supported by | | | |
| | | | cDNA | | | |
| 643 | Gma.742.1.A1_at | CD393482 | Unclassified - | | | |
| | | | Protein with | | | |
| | | | Unknown Function | | | |
| 644 | GmaAffx.89629.1.S1_s_at | CK606095 | Unclassified - | | | |
| | | | Protein with | | | |
| | | | Unknown Function | | | |
| 645 | Gma.12465.1.S1_a_at | CD415130 | Unclassified - | | | |
| | | | Hypothetical Protein | | | |
| | | | Supported by | | | |
| | | | cDNA | | | |
| 646 | Gma.11116.4.S1_s_at | CD408430 | Transporter | | | Aquaporin |
| | | | | | | (major intrinsic protein family) |
| 647 | Gma.2352.1.S1_at | AI443511 | Unclassified - | | | FOG: Predicted E3 |
| | | | Protein with | | | ubiquitin ligase |
| | | | Unknown Function | | | |
| 648 | Gma.6650.2.S1_at | BU762479 | Unclassified - | | | |
| | | | Protein with | | | |
| | | | Unknown Function | | | |
| 649 | GmaAffx.74289.1.S1_at | CA852521 | Transcription | Transcription | MYB | Transcription factor, |
| | | | | Factor | | Myb superfamily |
| 650 | Gma.9925.1.S1_at | AW307455 | No Homology to | | | |
| | | | Known Proteins | | | |
| 651 | Gma.14966.1.A1_at | CD416359 | Intracellular | | | Aquaporin |
| | | | Traffic | | | (major intrinsic protein family) |
| 652 | Gma.8077.1.S1_at | BU547877 | Cell Structure | | | |
| 653 | GmaAffx.30344.1.S1_at | AW349341 | Metabolism | | | Lipid phosphate |
| | | | | | | phosphatase and related |
| | | | | | | enzymes of the |
| | | | | | | PAP2 family |
| 654 | GmaAffx.70634.1.S1_at | BQ079384 | Protein | | | FOG: Predicted E3 |
| | | | Destination | | | ubiquitin ligase |
| | | | & Storage | | | |
| 655 | Gma.17347.1.S1_s_at | AW597676 | Transcription | Transcription | NAC | |
| | | | | Factor | Domain | |
| 656 | Gma.9606.1.S1_at | AB086392.1 | Transporter | | | Sucrose transporter |
| | | | | | | and related proteins |
| 657 | Gma.18088.1.S1_at | B1970254 | Unclassified - | | | |
| | | | Protein with | | | |
| | | | Unknown Function | | | |
| 658 | GmaAffx.91339.1.S1_at | CF807064 | Cell Structure | | | |
| 659 | Gma.14846.1.A1_at | AW310268 | No Homology to | | | |
| | | | Known Proteins | | | |
| 660 | Gma.5330.2.S1_at | BM092298 | Unclassified - | | | |
| | | | Hypothetical Protein | | | |
| | | | NOT Supported by | | | |
| | | | cDNA | | | |
| 661 | GmaAffx.90252.1.S1_s_at | CF805977 | Unclassified - | | | |
| | | | Hypothetical Protein | | | |
| | | | Supported by | | | |
| | | | cDNA | | | |
| 662 | GmaAffx.56272.1.S1_at | BU761188 | No Homology to | | | |
| | | | Known Proteins | | | |
| 663 | Gma.4368.1.S1_at | BQ080689 | Unclassified - | | | |
| | | | Protein with | | | |
| | | | Unknown Function | | | |
| 664 | GmaAffx.58555.1.S1_at | BQ298550 | Metabolism | Cytochrome | CYP82C | Cytochrome P450 CYP2 |
| | | | | P450 | | subfamily |
| 665 | Gma.11076.1.S1_at | BE658385 | Unclassified - | | | |
| | | | Hypothetical Protein | | | |
| | | | Supported by | | | |
| | | | cDNA | | | |
| 666 | GmaAffx.62710.1.S1_at | BE473384 | Unclassified - | | | |
| | | | Hypothetical Protein | | | |
| | | | NOT Supported by | | | |
| | | | cDNA | | | |
| 667 | GmaAffx.82105.1.S1_at | A1938690 | Transporter | | | Sucrose transporter |
| | | | | | | and related proteins |
| 668 | GmaAffx.50656.1.A1_at | B1970755 | No Homology to | | | |
| | | | Known Proteins | | | |
| 669 | GmaAffx.31548.1.S1_at | BG044632 | Transcription | Transcription | Unclassified | Transcription factor, |
| | | | | Factor | | Myb superfamily |
| 670 | GmaAffx.671.1.S1_at | CA785167 | Cell Structure | | | |
| 671 | GmaAffx.43620.1.S1_at | AW184956 | No Homology to | | | |
| | | | Known Proteins | | | |
| 672 | Gma.17586.1.S1_at | BU546955 | Unclassified - | | | Reticulon |
| | | | Hypothetical Protein | | | |
| | | | Supported by | | | |
| | | | cDNA | | | |
| 673 | GmaAffx.3363.1.S1_at | BE347006 | Unclassified - | | | |
| | | | Hypothetical Protein | | | |
| | | | NOT Supported by | | | |
| | | | cDNA | | | |
| 674 | Gma.12465.2.S1_at | BE611098 | Unclassified - | | | |
| | | | Hypothetical Protein | | | |
| | | | Supported by | | | |
| | | | cDNA | | | |
| 675 | GmaAffx.57341.1.S1_at | AW277978 | Transporter | | | Amino acid transporters |
| 676 | GmaAffx.77404.1.S1_at | CD393010 | Intracellular | | | GTPase Rab11/YPT3, |
| | | | Traffic | | | small G protein |
| | | | | | | superfamily |
| 677 | Gma.3576.2.S1_a_at | BI316058 | Unclassified - | | | Diadenosine |
| | | | Protein with | | | polyphosphate hydrolase |
| | | | Unknown Function | | | and related proteins of |
| | | | | | | the histidine triad |
| | | | | | | (HIT) family |
| 678 | Gma.612.1.A1_at | AI442942 | Transcription | Transcription | AP2/ | |
| | | | | Factor | EREBP | |
| 679 | GmaAffx.8013.1.S1_at | BE609266 | Unclassified - | | | |
| | | | Hypothetical Protein | | | |
| | | | NOT Supported by | | | |
| | | | cDNA | | | |
| 680 | Gma.10441.1.S1_s_at | BE803701 | Unclassified - | | | |
| | | | Protein with | | | |
| | | | Unknown Function | | | |
| 681 | GmaAffx.79401.1.S1_at | CA936959 | Transcription | Transcription | bZIP | |
| | | | | Factor | | |
| 682 | GmaAffx.46974.1.S1_at | BU081847 | Signal | Protein | | |
| | | | Transduction | Kinase | | |
| 683 | GmaAffx.23953.1.S1_s_at | BE658262 | Transporter | | | |
| 684 | Gma.13973.1.A1_at | CD412168 | No Homology to | | | |
| | | | Known Proteins | | | |
| 685 | GmaAffx.85469.1.S1_at | BI786397 | Unclassified - | | | |
| | | | Hypothetical Protein | | | |
| | | | NOT Supported by | | | |
| | | | cDNA | | | |
| 686 | Gma.7371.2.S1_a_at | BQ741076 | Cell Structure | | | Dynein light chain |
| | | | | | | type 1 |
| 687 | GmaAffx.7302.2.A1_s_at | BE820704 | No Homology to | | | |
| | | | Known Proteins | | | |
| 688 | Gma.13039.1.A1_at | CD399750 | Unclassified - | | | |
| | | | Hypothetical Protein | | | |
| | | | NOT Supported by | | | |
| | | | cDNA | | | |
| 689 | GmaAffx.81502.2.S1_at | BG041291 | Unclassified - | | | Uncharacterized |
| | | | Hypothetical Protein | | | conserved protein |
| | | | NOT Supported by | | | |
| | | | cDNA | | | |
| 690 | GmaAffx.63115.1.A1_at | BE823736 | Unclassified - | | | |
| | | | Hypothetical Protein | | | |
| | | | NOT Supported by | | | |
| | | | cDNA | | | |
| 691 | GmaAffx.53069.2.S1_at | AW309012 | No Homology to | | | |
| | | | Known Proteins | | | |
| 692 | GmaAffx.90242.1.S1_s_at | CF805967 | No Homology to | | | |
| | | | Known Proteins | | | |
| 693 | GmaAffx.50635.2.S1_at | AW733509 | Metabolism | Cytochrome | CYP707A | Cytochrome P450 |
| | | | | P450 | | CYP4/CYP19/CYP26 |
| | | | | | | subfamilies |
| 694 | GmaAffx.86667.1.S1_at | CA938472 | Unclassified - | | | |
| | | | Hypothetical Protein | | | |
| | | | Supported by | | | |
| | | | cDNA | | | |
| 695 | GmaAffx.71032.1.S1_at | AW278168 | Unclassified - | | | |
| | | | Hypothetical Protein | | | |
| | | | NOT Supported by | | | |
| | | | cDNA | | | |
| 696 | GmaAffx.76295.1.S1_at | BE347326 | Unclassified - | Hormone | | |
| | | | Protein with | Induced | | |
| | | | Unknown Function | | | |
| 697 | GmaAffx.79730.1.S1_at | BF325482 | Transcription | Transcription | Basic | |
| | | | | Factor | Helix- | |
| | | | | | Loop- | |
| | | | | | Helix | |
| | | | | | (bHLH) | |
| 698 | GmaAffx.23475.2.S1_s_at | AW620563 | Unclassified - | | | |
| | | | Hypothetical Protein | | | |
| | | | Supported by | | | |
| | | | cDNA | | | |
| 699 | Gma.7256.1.S1_at | L01447.1 | Transcription | Transcription | Basic | |
| | | | | Factor | Leucine | |
| | | | | | Zipper | |
| | | | | | (bZIP) | |
| 700 | Gma.13103.1.S1_at | CD392133 | Unclassified - | | | |
| | | | Hypothetical Protein | | | |
| | | | NOT Supported by | | | |
| | | | cDNA | | | |
| 701 | GmaAffx.27488.1.S1_at | AW311080 | Signal Transduction | MAPKKK | Raf | Tyrosine kinase |
| | | | | | | specific for activated |
| | | | | | | (GTP-bound) p21cdc42Hs |
| 702 | GmaAffx.6017.1.A1_at | BU550467 | Unclassified - | | | |
| | | | Hypothetical Protein | | | |
| | | | NOT Supported by | | | |
| | | | cDNA | | | |
| 703 | Gma.1951.2.S1_at | CA783485 | Protein | | | Serine |
| | | | Destination | | | carboxypeptidases |
| | | | & Storage | | | |
| 704 | GmaAffx.25912.1.S1_at | BU545028 | Unclassified - | | | |
| | | | Protein with | | | |
| | | | Unknown Function | | | |
| 705 | GmaAffx.69307.1.S1_at | BQ473855 | No Homology to | | | |
| | | | Known Proteins | | | |
| 706 | Gma.11194.2.S1_at | BM886635 | Energy | | | Thioredoxin |
| 707 | Gma.10100.1.S1_at | BE023275 | No Homology to | | | |
| | | | Known Proteins | | | |
| 708 | Gma.2827.1.S1_at | BG237560 | Unclassified - | | | FOG: PPR repeat |
| | | | Hypothetical Protein | | | |
| | | | Supported by | | | |
| | | | cDNA | | | |
| 709 | GmaAffx.26667.1.S1_at | BE806932 | No Homology to | | | |
| | | | Known Proteins | | | |
| 710 | Gma.8501.1.S1_at | AF184278.1 | Transcription | Transcription | Homeo- | Transcription factor |
| | | | | Factor | domain | HEX, contains HOX |
| | | | | | | and HALZ domains |
| 711 | GmaAffx.25068.2.A1_at | CD413732 | Metabolism | Amino Acid | | |
| | | | | Biosynthesis/ | | |
| | | | | Metabolism | | |
| 712 | Gma.13019.1.A1_s_at | CD392967 | Secondary | | | Hydroxyindole-O- |
| | | | Metabolism | | | methyltransferase and |
| | | | | | | related SAM-dependent |
| | | | | | | methyltransferases |
| 713 | GmaAffx.11496.1.S1_at | BE821691 | Transporter | Inorganic | Putative | Sulfate/bicarbonate/ |
| | | | | Solute Co- | sulfate | oxalate exchanger |
| | | | | transporters | transporter | SAT-1 and related |
| | | | | | family | transporters (SLC26 |
| | | | | | | family) |
| 714 | GmaAffx.52526.1.S1_at | BM085509 | Unclassified - | | | Uncharacterized |
| | | | Hypothetical Protein | | | conserved protein |
| | | | Supported by | | | |
| | | | cDNA | | | |
| 715 | GmaAffx.37316.1.S1_at | AW733720 | Unclassified - | | | |
| | | | Protein with | | | |
| | | | Unknown Function | | | |
| 716 | Gma.137.1.S1_at | AF022464.1 | Metabolism | Cytochrome | | Cytochrome P450 CYP2 |
| | | | | P450 | | subfamily |
| 717 | Gma.17594.2.S1_at | BM093196 | Metabolism | | | Cystathionine beta- |
| | | | | | | lyases/cystathionine |
| | | | | | | gamma-synthases |
| 718 | Gma.10873.1.S1_s_at | BQ079465 | No Homology to | | | |
| | | | Known Proteins | | | |
| 719 | GmaAffx.86212.1.S1_at | BG043474 | Unclassified - | | | |
| | | | Hypothetical Protein | | | |
| | | | NOT Supported by | | | |
| | | | cDNA | | | |
| 720 | GmaAffx.62153.1.S1_at | AW394575 | No Homology to | | | |
| | | | Known Proteins | | | |
| 721 | GmaAffx.37988.1.S1_at | BF324942 | No Homology to | | | |
| | | | Known Proteins | | | |
| 722 | GmaAffx.72807.1.S1_at | AW831487 | No Homology to | | | |
| | | | Known Proteins | | | |
| 723 | GmaAffx.70659.1.S1_at | A1495240 | No Homology to | | | |
| | | | Known Proteins | | | |
| 724 | GmaAffx.91358.1.S1_x_at | CF807083 | No Homology to | | | |
| | | | Known Proteins | | | |
| 725 | GmaAffx.70548.1.A1_at | CD411723 | No Homology to | | | |
| | | | Known Proteins | | | |
| 726 | GmaAffx.71038.1.S1_at | B1700350 | Transcription | Transcription | Homeo- | Transcription factor |
| | | | | Factor | domain | ME1S1 and related |
| | | | | | | HOX domain proteins |
| 727 | GmaAffx.15792.1.A1_at | AW101026 | No Homology to | | | |
| | | | Known Proteins | | | |
| 728 | Gma.3204.1.S1_at | AW509002 | Metabolism | | | Dehydrogenase kinase |
| 729 | Gma.4506.1.S1_at | BQ740943 | Unclassified - | | | |
| | | | Hypothetical Protein | | | |
| | | | NOT Supported by | | | |
| | | | cDNA | | | |
| 730 | GmaAffx.7838.3.S1_s_at | CA800410 | Metabolism | | | |
| 731 | Gma.5725.1.S1_at | BM886367 | Disease & Defense | | | |
| 732 | Gma.3901.1.S1_at | CD394788 | Protein | Storage | | |
| | | | Destination | Protein | | |
| | | | & Storage | | | |
| 733 | Gma.13845.1.A1_at | CD407676 | Unclassified - | | | |
| | | | Hypothetical Protein | | | |
| | | | Supported by | | | |
| | | | cDNA | | | |
| 734 | GmaAffx.1620.2.S1_at | BF324548 | Unclassified - | | | |
| | | | Hypothetical Protein | | | |
| | | | NOT Supported by | | | |
| | | | cDNA | | | |
| 735 | Gma.790.1.A1_s_at | BE820450 | Unclassified - | | | Predicted E3 |
| | | | Hypothetical Protein | | | ubiquitin ligase |
| | | | Supported by | | | |
| | | | cDNA | | | |
| 736 | GmaAffx.75459.1.S1_s_at | BE348034 | Protein | Storage | | |
| | | | Destination | Protein | | |
| | | | & Storage | | | |
| 737 | GmaAffx.58606.1.S1_at | BM526502 | Metabolism | | | |
| 738 | GmaAffx.78768.2.S1_at | A1930773 | Unclassified - | | | |
| | | | Hypothetical Protein | | | |
| | | | Supported by | | | |
| | | | cDNA | | | |
| 739 | Gma.9365.1.A1_at | CD418433 | No Homology to | | | |
| | | | Known Proteins | | | |
| 740 | Gma.1773.1.A1_at | CD404050 | Transcription | Transcription | Basic | |
| | | | | Factor | Helix- | |
| | | | | | Loop- | |
| | | | | | Helix | |
| | | | | | (bHLH) | |
| 741 | GmaAffx.89697.1.S1_s_at | CK606333 | Disease & Defense | | | |
| 742 | Gma.3617.1.S1_at | BE659297 | Cell Growth | | | |
| | | | & Division | | | |
| 743 | GmaAffx.74438.1.S1_at | BE440325 | Unclassified - | | | |
| | | | Hypothetical Protein | | | |
| | | | NOT Supported by | | | |
| | | | cDNA | | | |
| 744 | GmaAffx.40114.1.S1_at | B1973325 | Unclassified - | | | |
| | | | Hypothetical Protein | | | |
| | | | Supported by | | | |
| | | | cDNA | | | |
| 745 | GmaAffx.57432.1.S1_at | BE657443 | Unclassified - | | | |
| | | | Hypothetical Protein | | | |
| | | | NOT Supported by | | | |
| | | | cDNA | | | |
| 746 | Gma.13672.1.A1_at | CD403249 | No Homology to | | | |
| | | | Known Proteins | | | |
| 747 | Gma.8674.3.A1_at | B1469914 | Transporter | | | Predicted transporter |
| | | | | | | (major facilitator |
| | | | | | | superfamily) |
| 748 | Gma.7006.1.S1_at | AF516880.1 | Cell Structure | | | |
| 749 | GmaAffx.79283.1.S1_at | CA819076 | Unclassified - | | | |
| | | | Hypothetical Protein | | | |
| | | | NOT Supported by | | | |
| | | | cDNA | | | |
| 750 | GmaAffx.92964.1.S1_at | CF808689 | No Homology to | | | |
| | | | Known Proteins | | | |
| 751 | Gma.6333.2.S1_at | CD396361 | No Homology to | | | |
| | | | Known Proteins | | | |
| 752 | Gma.1426.1.A1_at | A1443364 | Metabolism | | | |
| 753 | Gma.2382.1.S1_x_at | BG789835 | Metabolism | | | |
| 754 | GmaAffx.92167.1.A1_s_at | CF807892 | Unclassified - | | | |
| | | | Hypothetical Protein | | | |
| | | | NOT Supported by | | | |
| | | | cDNA | | | |
| 755 | Gma.10999.1.S1_at | CA850772 | Disease & Defense | | | |
| 756 | Gma.11993.1.S1_at | BU551348 | Transporter | | | Transporter, ABC |
| | | | | | | superfamily (Breast |
| | | | | | | cancer resistance |
| | | | | | | protein) |
| 757 | Gma.2382.1.S1_s_at | BG789835 | Metabolism | | | |
| 758 | GmaAffx.72198.1.A1_at | BE823481 | Metabolism | | | UDP-glucuronosyl |
| | | | | | | and UDP-glucosyl |
| | | | | | | transferase |

[symbol]

indicates data missing or illegible when filed

TABLE 17
Root and drought related Gene names and sequence information Maize

| List of 273 genes | |
|---|---|
| Sequence ID | putative annotation |
| MZ00003364 | DNA-binding protein ABF1 - wild oat (fragment) {Avena fatua;} |
| MZ00003747 | putative molybdopterin synthase large subunit MOCS2B {Oryza sativa (japonica cultivar- |
| | group);} |
| MZ00003870 | putative NAC domain protein NAC1 {Oryza sativa (japonica cultivar-group);} |
| MZ00004215 | putative NAC domain protein NAC1 {Oryza sativa (japonica cultivar-group);} |
| MZ00004277 | putative bZIP transcription factor ABI5 {Oryza sativa (japonica cultivar-group);} |
| MZ00004593 | NAC transcription factor {Hordeum vulgare subsp. vulgare;} |
| MZ00004720 | O-methyltransferase ZRP4 (EC 2.1.1.—) (OMT). {Zea mays;} |
| MZ00004966 | putative OsNAC7 protein {Oryza sativa (japonica cultivar-group);} |
| MZ00005238 | probable peroxidase (EC 1.11.1.—) 1 precursor, anionic - maize {Zea mays;} |
| MZ00005590 | putative OsNAC1 protein {Oryza sativa (japonica cultivar-group);} |
| MZ00005994 | beta-expansin {Oryza sativa;} |
| MZ00006142 | putative bZIP transcription factor ABI5 {Oryza sativa (japonica cultivar-group);} |
| MZ00006161 | putative NAC2 protein {Oryza sativa (japonica cultivar-group);} |
| MZ00007492 | epoxide hydrolase-like protein - Arabidopsis thaliana {Arabidopsis thaliana;} |
| MZ00007636 | putative gibberellin 2-beta-dioxygenase 2 {Oryza sativa (japonica cultivar-group);} |
| MZ00008036 | putative NAC domain protein NAM {Oryza sativa (japonica cultivar-group);} |
| MZ00009501 | putative NAC2 protein {Oryza sativa (japonica cultivar-group);} |
| MZ00010122 | putative NAC domain protein NAC1 {Oryza sativa (japonica cultivar-group);} |
| MZ00010783 | putative NAC domain protein {Oryza sativa (japonica cultivar-group);} |
| MZ00013608 | jakalin homolog - barley {Hordeum vulgare;} |
| MZ00014591 | NAC2 protein-like {Oryza sativa (japonica cultivar-group);} |
| MZ00015899 | cinnamoyl CoA reductase - maize {Zea mays;} |
| MZ00016514 | Molybdenum cofactor synthesis protein 3 (Molybdopterin synthasesulfurylase) (MPT synthase |
| | sulfurylase). {Arabidopsis thaliana;} |
| MZ00016515 | Molybdenum cofactor synthesis protein 3 (Molybdopterin synthasesulfurylase) (MPT synthase |
| | sulfurylase). {Arabidopsis thaliana;} |
| MZ00016622 | putative NAC2 {Oryza sativa (japonica cultivar-group);} |
| MZ00016927 | knotted class 1 homeodomain protein liguleless3 {Zea mays;} |
| MZ00017021 | putative transcription factor {Oryza sativa (japonica cultivar-group);} |
| MZ00017154 | putative DNA binding protein ACBF {Arabidopsis thaliana;} |
| MZ00017390 | G-box binding factor 1 - maize {Zea mays;} |
| MZ00017701 | CCAAT-binding transcription factor subunit A (CBF-A) (NF-Y proteinchain B) (NF-YB) (CAAT- |
| | box DNA binding protein subunit B). {Zea mays;} |
| MZ00017884 | NAC2 protein-like {Oryza sativa (japonica cultivar-group);} |
| MZ00018433 | putative CTR1-like kinase kinase kinase {Oryza sativa (japonica cultivar-group);} |
| MZ00018509 | homeodomain leucine zipper protein {Oryza sativa (japonica cultivar-group);} |
| MZ00018690 | gibberellin 20-dioxygenase (EC 1.14.11.—) (clone S39A) [similarity] - wheat {Triticum aestivum;} |
| MZ00018988 | putative acetylornithine aminotransferase {Oryza sativa (japonica cultivar-group);} |
| MZ00019635 | putative multidrug resistance p-glycoprotein {Oryza sativa (japonica cultivar-group);} |
| MZ00019674 | NAC transcription factor {Hordeum vulgare subsp. vulgare;} |
| MZ00020252 | putative NAC2 protein {Oryza sativa (japonica cultivar-group);} |
| MZ00020537 | putative regulatory protein {Oryza sativa (japonica cultivar-group);} |
| MZ00021106 | putative wall-associated serine/threonine kinase {Oryza sativa (japonica cultivar-group);} |
| MZ00021464 | putative xyloglucan endo-1,4-beta-D-glucanase {Oryza sativa (japonica cultivar-group);} |
| MZ00022008 | transcription factor DREB {Oryza sativa (japonica cultivar-group);} |
| MZ00022039 | putative DNA binding protein ACBF {Arabidopsis thaliana;} |
| MZ00022254 | DNA-binding protein ABF1 - wild oat (fragment) {Avena fatua;} |
| MZ00022590 | putative NAC domain protein NAC1 {Oryza sativa (japonica cultivar-group);} |
| MZ00023290 | TA1 protein {Oryza sativa (japonica cultivar-group);} |
| MZ00023972 | OsNAC3 protein {Oryza sativa (japonica cultivar-group);} |
| MZ00023973 | putative NAC-domain protein {Oryza sativa (japonica cultivar-group);} |
| MZ00024843 | putative zinc finger transcription factor {Oryza sativa (japonica cultivar-group);} |
| MZ00025478 | cinnamyl-alcohol dehydrogenase (EC 1.1.1.195) - maize {Zea mays;} |
| MZ00025512 | CCAAT-binding transcription factor subunit A (CBF-A) (NF-Y proteinchain B) (NF-YB) (CAAT- |
| | box DNA binding protein subunit B). {Zea mays;} |
| MZ00025666 | putative acetylornithine aminotransferase {Oryza sativa (japonica cultivar-group);} |
| MZ00026366 | Chalcone--flavonone isomerase (EC 5.5.1.6) (Chalcone isomerase). {Zea mays;} |
| MZ00026596 | ethylene responsive element binding factor3 {Oryza sativa (japonica cultivar-group);} |
| MZ00026661 | putative NAC-domain protein {Oryza sativa (japonica cultivar-group);} |
| MZ00027155 | putative development regulation gene OsNAC4 {Oryza sativa (japonica cultivar-group);} |
| MZ00027281 | putative CCAAT-binding transcription factor {Oryza sativa (japonica cultivar-group);} |
| MZ00027530 | homeodomain leucine zipper protein {Oryza sativa (japonica cultivar-group);} |
| MZ00027738 | probable molybdopterin synthase small chain F28M11.20 [similarity] - Arabidopsis thaliana |
| | {Arabidopsis thaliana;} |
| MZ00027872 | putative proline oxidase {Oryza sativa (japonica cultivar-group);} |
| MZ00028118 | transcription factor OBF3.2 - maize {Zea mays;} |
| MZ00028268 | NAC1 {Medicago truncatula;} |
| MZ00028302 | putative NAC2 protein {Oryza sativa (japonica cultivar-group);} |
| MZ00029259 | DREB-like protein {Zea mays;} |
| MZ00029319 | putative transcription factor {Oryza sativa (japonica cultivar-group);} |
| MZ00030166 | OsNAC5 protein [imported] - rice {Oryza sativa;} |
| MZ00030256 | epoxide hydrolase-like protein - Arabidopsis thaliana {Arabidopsis thaliana;} |
| MZ00030973 | putative NAC-domain protein {Oryza sativa (japonica cultivar-group);} |
| MZ00033479 | putative development regulation gene OsNAC4 {Oryza sativa (japonica cultivar-group);} |
| MZ00033711 | putative NAC-domain protein {Oryza sativa (japonica cultivar-group);} |
| MZ00035723 | unnamed protein product; GST I (AA 1-214) {Zea mays;} |
| MZ00035882 | putative NAC domain protein NAC1 {Oryza sativa (japonica cultivar-group);} |
| MZ00035947 | putative NAC-domain protein {Oryza sativa (japonica cultivar-group);} |
| MZ00035980 | OsNAC6 protein [imported] - rice {Oryza sativa;} |
| MZ00036019 | putative NAC-domain protein {Oryza sativa (japonica cultivar-group);} |
| MZ00036040 | putative OsNAC1 protein {Oryza sativa (japonica cultivar-group);} |
| MZ00036063 | putative development regulation gene OsNAC4 {Oryza sativa (japonica cultivar-group);} |
| MZ00036461 | NAC transcription factor {Hordeum vulgare subsp. vulgare;} |
| MZ00036503 | OsNAC6 protein [imported] - rice {Oryza sativa;} |
| MZ00036989 | putative aldehyde dehydrogenase MIS1 {Zea mays;} |
| MZ00038050 | putative NAC2 protein {Oryza sativa (japonica cultivar-group);} |
| MZ00038419 | Transcription factor 1CE1 (Inducer of CBF expression 1) (Basic helix-loop-helix protein 116) |
| | (bHLH116) (AtbHLH116). {Arabidopsis thaliana;} |
| MZ00040653 | glutathione S-transferase GST 29 {Zea mays;} |
| MZ00041439 | probable lipase - maize {Zea mays;} |
| MZ00041704 | Delta 1-pyrroline-5-carboxylate synthetase (P5CS) [Includes: Glutamate5-kinase (EC 2.7.2.11) |
| | (Gamma-glutamyl kinase) (GK); Gamma-glutamylphosphate reductase (GPR) (EC 1.2.1.41) |
| | (Glutamate-5-semialdehydedehydrogenase) (Glutamyl-gamma-semialdehyde dehydroge |
| MZ00041803 | zinc finger protein {Oryza sativa (japonica cultivar-group);} |
| MZ00043228 | OsNAC6 protein [imported] - rice {Oryza sativa;} |
| MZ00043782 | CCAAT-binding transcription factor subunit A (CBF-A) (NF-Y proteinchain B) (NF-YB) (CAAT- |
| | box DNA binding protein subunit B). {Zea mays;} |
| MZ00043783 | CCAAT-binding transcription factor subunit A (CBF-A) (NF-Y proteinchain B) (NF-YB) (CAAT- |
| | box DNA binding protein subunit B). {Zea mays;} |
| MZ00044271 | putative proline oxidase {Oryza sativa (japonica cultivar-group);} |
| MZ00044858 | expansin-like protein A {Oryza sativa;} |
| MZ00046814 | zeaxanthin epoxidase {Triticum aestivum;} |
| MZ00046973 | putative glucanase {Oryza sativa (japonica cultivar-group);} |
| MZ00048054 | DREB-like protein {Zea mays;} |
| MZ00048170 | putative NAC2 protein {Oryza sativa (japonica cultivar-group);} |
| MZ00049187 | putative CCAAT-binding transcription factor subunit A(CBF-A) {Oryza sativa (japonica cultivar- |
| | group);} |
| MZ00049453 | NAC2 protein-like {Oryza sativa (japonica cultivar-group);} |
| MZ00049454 | putative NAC domain protein {Oryza sativa (japonica cultivar-group);} |
| MZ00049520 | NAC2 protein-like {Oryza sativa (japonica cultivar-group);} |
| MZ00050952 | putative OsNAC7 protein {Oryza sativa (japonica cultivar-group);} |
| MZ00051037 | putative bZIP transcription factor ABI5 {Oryza sativa (japonica cultivar-group);} |
| MZ00055816 | putative NAC-domain protein {Oryza sativa (japonica cultivar-group);} |
| MZ00057236 | putative NAC-domain protein {Oryza sativa (japonica cultivar-group);} |
| MZ00057269 | caffeoyl CoA 3-O-methyltransferase {Zea mays;} |
| MZ00022124 | Putative AP2/EREBP transcription factor related cluster |
| MZ00004104 | putative polyprotein {Zea mays;} |
| MZ00004533 | putative CCAAT-binding transcription factor {Oryza sativa (japonica cultivar-group);} |
| MZ00005099 | DRE-binding protein 1A {Oryza sativa (japonica cultivar-group);} |
| MZ00008076 | hypothetical protein {Oryza sativa (japonica cultivar-group);} |
| MZ00010827 | CCAAT-box-binding transcription factor-like protein {Oryza sativa (japonica cultivar-group);} |
| MZ00012038 | CCAAT-box transcription factor -related {Oryza sativa (japonica cultivar-group);} |
| MZ00012977 | DRE-binding protein 1A {Oryza sativa (japonica cultivar-group);} |
| MZ00015861 | AP2 transcriptional activator DRF1.3 {Hordeum vulgare;} |
| MZ00015941 | putative CCAAT displacement protein {Oryza sativa (japonica cultivar-group);} |
| MZ00001289 | AP2/EREBP transcription factor-like protein {Oryza sativa (japonica cultivar-group);} |
| MZ00021137 | putative CCAAT-binding transcription factor {Oryza sativa (japonica cultivar-group);} |
| MZ00050079 | AP2/EREBP transcription factor-like protein {Oryza sativa (japonica cultivar-group);} |
| MZ00031905 | putative CCAAT displacement protein {Oryza sativa (japonica cultivar-group);} |
| MZ00037869 | AP2 transcriptional activator DRF1.3 {Hordeum vulgare;} |
| MZ00041923 | CCAAT-box transcription factor complex WHAP5 {Triticum aestivum;} |
| MZ00044670 | putative AP2/EREBP transcription factor {Oryza sativa (japonica cultivar-group);} |
| MZ00031604 | CCAAT-box transcription factor -related {Oryza sativa (japonica cultivar-group);} |
| MZ00019061 | CCAAT-box transcription factor -related {Oryza sativa (japonica cultivar-group);} |
| MZ00003515 | Putative AP2/EREBP transcription factor related cluster |
| MZ00001373 | putative F-box protein Fbl2 {Oryza sativa (japonica cultivar-group);} |
| MZ00001869 | putative organic cation transporter {Oryza sativa (japonica cultivar-group);} |
| MZ00005490 | Beta-fructofuranosidase 1 precursor (EC 3.2.1.26) (Sucrose-6-phosphatehydrolase 1) (Invertase |
| | 1). {Zea mays;} |
| MZ00005891 | Subtilisin inhibitor 1 (AS1-1) [Contains: Subtilisin inhibitor 11(AS1-11)]. {Vigna angularis;} |
| MZ00006368 | ABC1 family protein kinase-like protein {Oryza sativa (japonica cultivar-group);} |
| MZ00006817 | OSJNBa0069D17.7 {Oryza sativa (japonica cultivar-group);} |
| MZ00007968 | putative bZIP transcription factor ABI5 {Oryza sativa (japonica cultivar-group);} |
| MZ00009288 | Putative anion transporter {Oryza sativa (japonica cultivar-group);} |
| MZ00011868 | putative transmembrane protein {Oryza sativa (japonica cultivar-group);} |
| MZ00012450 | putative amino acid transport protein {Oryza sativa (japonica cultivar-group);} |
| MZ00013901 | auxin-repressed protein {Robinia pseudoacacia;} |
| MZ00013921 | heat shock protein hsp70. {Zea mays;} |
| MZ00014257 | Glucose-1-phosphate adenylyltransferase large subunit 2, chloroplastprecursor (EC 2.7.7.27) |
| | (ADP-glucose synthase) (ADP-glucosepyrophosphorylase) (AGPASE S) (Alpha-D-glucose-1- |
| | phosphate adenyltransferase). {-TRUNCATED- |
| MZ00014872 | OSJNBa0013K16.6 {Oryza sativa (japonica cultivar-group);} |
| MZ00015469 | putative peroxidase {Oryza sativa (japonica cultivar-group);} |
| MZ00015929 | putative 24 kDa seed maturation protein {Oryza sativa (japonica cultivar-group);} |
| MZ00015996 | putative Ubiquitin ligase SINAT5 {Oryza sativa (japonica cultivar-group);} |
| MZ00016125 | protein phosphatase 2C-like protein {Oryza sativa (japonica cultivar-group);} |
| MZ00016532 | putative zinc finger transcription factor {Oryza sativa (japonica cultivar-group);} |
| MZ00016971 | alpha-expansin 1 {Zea mays;} |
| MZ00017753 | glutathione transferase (EC 2.5.1.18) - maize {Zea mays;} |
| MZ00019036 | putative protein phosphatase 2C {Oryza sativa (japonica cultivar-group);} |
| MZ00019538 | unknown protein {Oryza sativa (japonica cultivar-group);} |
| MZ00020337 | hypothetical protein F1511.13 [imported] - Arabidopsis thaliana {Arabidopsis thaliana;} |
| MZ00020918 | putative raffinose synthase or seed imbibition protein {Oryza sativa (japonica cultivar-group);} |
| MZ00021179 | Putative starch synthase {Oryza sativa (japonica cultivar-group);} |
| MZ00021828 | major facilitator superfamily antiporter {Oryza sativa (japonica cultivar-group);} |
| MZ00022611 | putative sucrosephosphate synthase {Oryza sativa (japonica cultivar-group);} |
| MZ00023450 | zinc finger protein {Oryza sativa (japonica cultivar-group);} |
| MZ00023744 | putative glycosyl hydrolase family 35 (beta-galactosidase) {Oryza sativa (japonica cultivar- |
| | group);} |
| MZ00024083 | OSJNBa0024J22.14 {Oryza sativa (japonica cultivar-group);} |
| MZ00024840 | OSJNBa0004N05.16 {Oryza sativa (japonica cultivar-group);} |
| MZ00025001 | Putative anion transporter {Oryza sativa (japonica cultivar-group);} |
| MZ00025184 | contains ESTs AU081301(E20138), C99280(E10593) similar to Arabidopsis thaliana |
| | chromosome 2, F12C20.11 unknown protein {Oryza sativa (japonica cultivar-group);} |
| MZ00025596 | putative delta 1 pyrroline-5-carboxylate synthetase {Oryza sativa (japonica cultivar-group);} |
| MZ00026383 | sucrose synthase 3 {Zea mays;} |
| MZ00026642 | probable lipase - maize {Zea mays;} |
| MZ00026815 | putative oxalate oxidase {Oryza sativa (japonica cultivar-group);} |
| MZ00026870 | RISBZ5 {Oryza sativa (japonica cultivar-group);} |
| MZ00027872 | putative proline oxidase {Oryza sativa (japonica cultivar-group);} |
| MZ00028712 | putative thioredoxin {Oryza sativa (japonica cultivar-group);} |
| MZ00029301 | beta-expansin {Oryza sativa;} |
| MZ00030567 | expansin - rice {Oryza sativa;} |
| MZ00030937 | OSJNBa0069D17.7 {Oryza sativa (japonica cultivar-group);} |
| MZ00031064 | hypothetical protein {Oryza sativa (japonica cultivar-group);} |
| MZ00031622 | oligopeptide transporter OPT-like {Oryza sativa (japonica cultivar-group);} |
| MZ00034968 | Beta-glucosidase aggregating factor related cluster |
| MZ00035433 | putative protein phosphatase type-2C {Oryza sativa (japonica cultivar-group);} |
| MZ00035565 | Catalase isozyme 3 (EC 1.11.1.6). {Zea mays;} |
| MZ00035785 | beta-glucosidase aggregating factor precursor {Zea mays;} |
| MZ00035829 | Alkaline alpha galactosidase 1 related cluster |
| MZ00036333 | putative Sip1 protein {Oryza sativa (japonica cultivar-group);} |
| MZ00036708 | cystatin - maize {Zea mays;} |
| MZ00036823 | AT3g23600/MDB19_9 {Arabidopsis thaliana;} |
| MZ00037273 | unnamed protein product; ESTs D39300(R3292), AU030751(E60187) correspond to a region of |
| | the predicted gene. Similar to peroxidase ATP6a. (X98774) {Oryza sativa (japonica cultivar- |
| | group);} |
| MZ00037469 | probable lipase - maize {Zea mays;} |
| MZ00037481 | putative auxin response transcription factor {Oryza sativa (japonica cultivar-group);} |
| MZ00040770 | hypothetical protein {Oryza sativa (japonica cultivar-group);} |
| MZ00041427 | Catalase isozyme 3 (EC 1.11.1.6). {Zea mays;} |
| MZ00042123 | putative protein phosphatase type-2C {Oryza sativa (japonica cultivar-group);} |
| MZ00042215 | beta-expansin 6 {Zea mays;} |
| MZ00042638 | catalase isozyme 3 (EC 1.11.1.6) {Zea mays;} |
| MZ00043256 | sorbitol transporter {Malus × domestica;} |
| MZ00043643 | Putative anion transporter {Oryza sativa (japonica cultivar-group);} |
| MZ00043808 | putative delta 1 pyrroline-5-carboxylate synthetase {Oryza sativa (japonica cultivar-group);} |
| MZ00043924 | RTNLB20 {Zea mays;} |
| MZ00044103 | putative oxalate oxidase {Oryza sativa (japonica cultivar-group);} |
| MZ00044579 | putative protein kinase {Oryza sativa (japonica cultivar-group);} |
| MZ00044859 | putative heat shock protein 16.9 {Oryza sativa (japonica cultivar-group);} |
| MZ00047578 | putative myb transcription factor {Oryza sativa (japonica cultivar-group);} |
| MZ00051675 | CIPK-like protein {Oryza sativa (japonica cultivar-group);} |
| MZ00051941 | hypothetical protein F22O13.20 - Arabidopsis thaliana {Arabidopsis thaliana;} |
| MZ00054974 | putative protein phosphatase 2C {Oryza sativa (japonica cultivar-group);} |
| MZ00007449 | dehydrin {Hordeum vulgare subsp. vulgare;} |
| MZ00009861 | Late embryogenesis abundant protein Lea14-A. {Gossypium hirsutum;} |
| MZ00014055 | putative late embryogenesis abundant protein {Oryza sativa (japonica cultivar-group);} |
| MZ00024561 | putative late embryogenesis abundant protein {Oryza sativa (japonica cultivar-group);} |
| MZ00025109 | Dehydrin DHN1 (M3) (RAB-17 protein). {Zea mays;} |
| MZ00028039 | Late embryogenesis abundant protein EMB564. {Zea mays;} |
| MZ00034443 | dehydrin-like protein {Zea mays;} |
| MZ00037212 | Late embryogenesis abundant protein, group 3 (LEA). {Zea mays;} |
| MZ00037250 | putative late embryogenesis abundant protein {Oryza sativa (japonica cultivar-group);} |
| MZ00038512 | putative late embryogenesis abundant protein {Oryza sativa (japonica cultivar-group);} |
| MZ00040778 | Late embryogenesis abundant protein, group 3 (LEA). {Zea mays;} |
| MZ00041369 | putative late embryogenesis abundant protein {Oryza sativa (japonica cultivar-group);} |
| MZ00042357 | Late embryogenesis abundant protein, group 3 (LEA). {Zea mays;} |
| MZ00043424 | dehydrin-like protein {Zea mays;} |
| MZ00044290 | Late embryogenesis abundant protein EMB564. {Zea mays;} |
| MZ00046901 | dehydrin {Hordeum vulgare subsp. vulgare;} |
| MZ00000002 | putative OsNAC7 protein {Oryza sativa (japonica cultivar-group);} |
| MZ00000032 | putative OsNAC7 protein {Oryza sativa (japonica cultivar-group);} |
| MZ00000937 | putative bHLH084 transcription factor {Arabidopsis thaliana;} |
| MZ00001016 | zeaxanthin epoxidase {Oryza sativa (japonica cultivar-group);} |
| MZ00001154 | putative delta 1 pyrroline-5-carboxylate synthetase {Oryza sativa (japonica cultivar-group);} |
| MZ00001345 | putative RING zinc finger protein {Oryza sativa (japonica cultivar-group);} |
| MZ00001666 | VP1/ABI3 family regulatory protein-like {Oryza sativa (japonica cultivar-group);} |
| MZ00003193 | putative OsNAC7 protein {Oryza sativa (japonica cultivar-group);} |
| MZ00017569 | CCAAT-box binding factor HAP5 homolog {Daucus carota;} |
| MZ00033883 | DREB-like protein {Zea mays;} |
| MZ00047852 | CCAAT-box transcription factor -related {Oryza sativa (japonica cultivar-group);} |
| AF261941 | germin-like protein 1 (GLP1) |
| AF261942 | germin-like protein 2 (GLP2) |
| AF413202 | Maize D8 |
| A1964498 | NADPH oxidase |
| AJ441084 | proton-exporting ATPase (mha3 gene) |
| AJ539534 | proton-exporting ATPase (mha4 gene). |
| AM238660 | beta-D-glucosidase |
| AY072297 | lignin05 |
| AY105196 | Rice GID1, GA receptor? |
| AY394010 | germin- like |
| BM382361 | NADPH oxidase |
| BM952819 | lignin03 |
| BQ619043 | lignin04 |
| BQ779300 | Barley ABAP1, ABA receptor? |
| BT016251 | unknown(GenBank 13194668) |
| CA827623 | P5CS2 |
| CD438883 | AB12 |
| CO440079 | CYP707A |
| CO445178 | CYP707A |
| CO453686 | CYP707A |
| CO462203 | lignin03 |
| CV072008 | vp14 |
| CX725402 | NADPH oxidase |
| D88451 | AAO3 |
| D88452 | AAO2 |
| DR824591 | lignin02 |
| DR958238 | era1 |
| DR963625 | vp1 |
| DR967411 | P5CR |
| DV032363 | vp14 |
| DV032810 | proline2 |
| DV527896 | CYP707A |
| DV528193 | UDP-glucosyltransferase BX9 (Bx9) |
| DV533388 | vp14 |
| EB484179 | NADPH oxidase |
| EB816224 | vp14 |
| U08984 | plasma-membrane H+ ATPase (Zmpma1) |
| U15781 | xyloglucan endo-transglycosylase |
| U15964 | xyloglucan endo-transglycosylase |
| U32579 | ABA GA? |
| U81960 | KAPP |
| X74217 | beta-D-glucosidase |
| X85805 | plasma membrane H+ ATPase |
| X98083 | lignin01 |
| Y13733 | lignin02 |
| Y13734 | lignin01 |
| AW499206 | lignin04 |
| BE056037 | lignin03 |
| BM378087 | AB11 |

TABLE 18

| Soy Gene ID | Maize Seq ID | % similarity |
|---|---|---|
| Gma.3888.2.S1_at | MZ00015192 | 89.36 |
| Gma.3888.2.S1_at | MZ00015192 | 93.75 |
| Gma.4252.1.S1_at | MZ00015682 | 82.99 |
| GmaAffx.60984.1.S1_at | MZ00015008 | 86.36 |
| GmaAffx.77832.1.S1_at | MZ00025962 | 87.34 |
| GmaAffx.89624.1.A1_s_at | MZ00007968 | 80.9 |
| GmaAffx.93591.1.S1_s_at | MZ00034172 | 93.55 |

TABLE 19

| Soy Gene ID | Maize seq ID | % similarity |
|---|---|---|
| Gma.13146.1.S1_at | MZ00023844 | 100 |
| Gma.13187.1.S1_at | MZ00002154 | 100 |
| Gma.13433.1.A1_at | MZ00037202 | 96 |
| Gma.1521.1.S1_at | MZ00023656 | 87.91 |
| Gma.1521.1.S1_at | MZ00043018 | 92.19 |
| Gma.1521.1.S1_at | MZ00043041 | 86.67 |
| Gma.1521.1.S1_at | MZ00037863 | 85 |
| Gma.1521.1.S1_at | MZ00019947 | 84.11 |
| Gma.1521.1.S1_at | MZ00023842 | 89.06 |
| Gma.1521.1.S1_at | MZ00049360 | 89.06 |
| Gma.1521.1.S1_at | MZ00038354 | 88.24 |
| Gma.1521.1.S1_at | MZ00038695 | 90.38 |
| Gma.17985.1.S1_at | MZ00056420 | 92.86 |
| Gma.1951.2.S1_at | MZ00017283 | 100 |
| Gma.2488.1.S1_at | MZ00030431 | 88.46 |
| Gma.3888.2.S1_at | MZ00015192 | 89.36 |
| Gma.3888.2.S1_at | MZ00015192 | 93.75 |
| Gma.4252.1.S1_at | MZ00015682 | 82.99 |
| Gma.6763.1.A1_at | MZ00016771 | 79.9 |
| Gma.8896.1.S1_at | MZ00015673 | 82.86 |
| GmaAffx.11276.1.S1_s_at | MZ00026818 | 91.3 |
| GmaAffx.11276.1.S1_s_at | MZ00044104 | 91.3 |
| GmaAffx.45241.1.S1_at | MZ00036400 | 100 |
| GmaAffx.52826.3.S1_at | MZ00020299 | 82.72 |
| GmaAffx.72630.1.S1_at | MZ00017283 | 93.1 |
| GmaAffx.89624.1.A1_s_at | MZ00006012 | 83.82 |
| GmaAffx.89624.1.A1_s_at | MZ00007968 | 80.9 |
| GmaAffx.89567.1.A1_at | MZ00034278 | 84.29 |
| GmaAffx.91346.1.S1_x_at | MZ00026818 | 92.86 |
| GmaAffx.91346.1.S1_x_at | MZ00044104 | 92.86 |
| GmaAffx.91346.1.S1_at | MZ00026818 | 92.86 |
| GmaAffx.91346.1.S1_at | MZ00044104 | 92.86 |
| GmaAffx.91731.1.S1_s_at | MZ00018436 | 85.42 |
| GmaAffx.92127.1.S1_s_at | MZ00050071 | 96 |

TABLE 20

| Soy gene ID | Maize Seq ID | % Similarity |
|---|---|---|
| Gma.15469.1.S1_at | MZ00007717 | 84.09 |
| Gma.15469.1.S1_at | MZ00007717 | 85.25 |
| Gma.16196.1.S1_at | MZ00027281 | 100 |
| Gma.17921.2.S1_s_at | MZ00037218 | 85.4 |
| Gma.17921.2.S1_s_at | MZ00026383 | 85.4 |
| Gma.17921.2.S1_s_at | MZ00026383 | 77.88 |
| Gma.3888.2.S1_at | MZ00015192 | 89.36 |
| Gma.3888.2.S1_at | MZ00015192 | 93.75 |
| Gma.5139.1.S1_at | MZ00035723 | 91.89 |
| Gma.5309.1.S1_at | MZ00005011 | 86 |
| Gma.5586.2.S1_at | MZ00027281 | 100 |
| Gma.6288.1.S1_at | MZ00040859 | 92.59 |
| Gma.8946.1.S1_at | MZ00010122 | 95.83 |
| GmaAffx.30428.1.S1_at | MZ00013921 | 84.97 |
| GmaAffx.30428.1.S1_at | MZ00013921 | 85.92 |
| GmaAffx.30428.1.S1_at | MZ00041287 | 85.39 |
| GmaAffx.70008.1.S1_at | MZ00047578 | 84.13 |
| GmaAffx.74323.1.S1_at | MZ00022560 | 100 |
| GmaAffx.89624.1.A1_s_at | MZ00018405 | 86.46 |
| GmaAffx.89624.1.A1_s_at | MZ00006012 | 83.82 |
| GmaAffx.89624.1.A1_s_at | MZ00007968 | 80.9 |
| GmaAffx.92192.1.S1_s_at | MZ00017681 | 83.64 |

TABLE 21

| Soy gene ID | maize Seq ID | % similarity |
|---|---|---|
| Gma.15469.1.S1_at | MZ00007717 | 84.09 |
| Gma.15469.1.S1_at | MZ00007717 | 85.25 |
| Gma.15773.1.A1_at | MZ00032969 | 95.83 |
| Gma.16196.1.S1_at | MZ00027281 | 100 |
| Gma.17921.2.S1_s_at | MZ00037218 | 85.4 |
| Gma.17921.2.S1_s_at | MZ00026383 | 85.4 |
| Gma.17921.2.S1_s_at | MZ00026383 | 77.88 |
| Gma.3888.2.S1_at | MZ00015192 | 89.36 |
| Gma.3888.2.S1_at | MZ00015192 | 93.75 |
| Gma.4643.1.S1_at | MZ00010122 | 100 |
| Gma.5309.1.S1_at | MZ00005011 | 86 |
| Gma.5468.2.S1_a_at | MZ00031424 | 90.62 |
| Gma.5586.2.S1_at | MZ00027281 | 100 |
| Gma.6528.2.S1_at | MZ00044238 | 100 |
| Gma.8946.1.S1_at | MZ00010122 | 95.83 |
| Gma.9856.1.S1_at | MZ00009779 | 100 |
| GmaAffx.14279.1.S1_at | MZ00012085 | 100 |
| GmaAffx.30428.1.S1_at | MZ00013921 | 84.97 |
| GmaAffx.30428.1.S1_at | MZ00013921 | 85.92 |
| GmaAffx.30428.1.S1_at | MZ00041287 | 85.39 |
| GmaAffx.70008.1.S1_at | MZ00047578 | 84.13 |
| GmaAffx.74323.1.S1_at | MZ00022560 | 100 |
| GmaAffx.79121.1.S1_at | MZ00016532 | 86.36 |
| GmaAffx.81388.1.S1_at | MZ00044869 | 82.58 |
| GmaAffx.81388.1.S1_at | MZ00035278 | 84.75 |
| GmaAffx.81388.1.S1_at | MZ00038106 | 82.61 |
| GmaAffx.87922.1.S1_at | MZ00019214 | 88.37 |
| GmaAffx.88251.1.S1_s_at | MZ00016490 | 90.74 |
| GmaAffx.88251.1.S1_s_at | MZ00016490 | 85.94 |
| GmaAffx.89624.1.A1_s_at | MZ00018405 | 86.46 |
| GmaAffx.89624.1.A1_s_at | MZ00006012 | 83.82 |
| GmaAffx.89624.1.A1_s_at | MZ00007968 | 80.9 |
| GmaAffx.92127.1.S1_s_at | MZ00050071 | 96 |
| GmaAffx.92192.1.S1_s_at | MZ00017681 | 83.64 |

TABLE 22
List of Gene constructs for functional characterization - Drought tolerance studies

| | | | | | |
|---|---|---|---|---|---|
| Sr. # | Name | Proposed functional Category | Promoter used | Transgenic seed status | SEQ ID NO. |

Group I - Drought responsive and root related genes already cloned and transfomed in SOYBEAN for functional analysis

| | | | | | |
|---|---|---|---|---|---|
| 1 | GmGalactinol synthase (GmGOL) | Metabolism | ABRC3 | T2 | 481 |
| 2 | GmGalactinol synthase (GmGOL) | Metabolism | rd29 | T2 | 481 |
| 3 | GmTrehalose phosphate synthase (GmTPS) | Metabolism | ABRC3 | T1 | 482 |
| 4 | GmTrehalose phosphate synthase (GmTPS) | Metabolism | rd29 | T1 | 482 |
| 5 | GmPotassium vacular transporter (GmCHX) | Metabolism | ABRC3 | T1 | 483 |
| 6 | GmPotassium vacular transporter (GmCHX) | Metabolism | rd29 | T1 | 483 |
| 7 | GmMaturation protein (GmPM30) | LEA protein | ABRC3 | T2 | 484 |
| 8 | Soybean homolog of AtABF3 (arabidopsis) | TF | ABRC3 | T2 | 485 |
| 9 | LeASR1 (tomato) | Metabolism ABA related | ABRC3 | T1 | 486 |
| 10 | Soybean homolog of AtPLDdb (arabidopsis) | Metabolism | ABRC3 | T2 | 488 |
| 11 | AtErecta (arabidopsis) | Signalling | ABRC3 | T1 | 489 |

Group II - Drought responsive and root related genes - Cloned and transfomed in ARABIDOPSIS for functional analysis screening

| | | | | | |
|---|---|---|---|---|---|
| 1 | GmNAC2 | TF | 35S, rd29 | T1 | 490 |
| 2 | GmNAC3 | TF | 35S | T3 | 491 |
| 3 | GmNAC4 | TF | 35S | T3 | 492 |
| 4 | GmNAC5 | TF | 35S, rd29 | T2 | 493 |
| 5 | GmNAC12 | TF | rd29, sub2 | T2 | 494 |
| 6 | GmNAC13 | TF | rd29, sub2 | T2 | 495 |
| 7 | GmNAC20 | TF | rd29, sub2 | T2 | 496 |
| 8 | GmNAC28 | TF | rd29, sub2 | T2 | 497 |
| 9 | GmC2H2 | TF | 35S, rd29 | T3 | 498 |
| 10 | GmDof27 (C2C2 zinc finger) | TF | 35S, rd29 | T3 | 499 |
| 11 | GmbZip35 | TF | 35S, rd29 | T2 | 500 |
| 12 | AtMBF1C | Transcription co activator | ABRC3 | T2 | 511 |
| 13 | AtSKU5 | Related to Coppeer oxidases | ABRC3 | T2 | 501 |
| 14 | GmVTE5 | Metabolism (ROS quenching) | ABRC3 | T2 | 502 |
| 15 | AtVTC2 | Metabolism (ROS quenching) | ABRC3 | T2 | 503 |
| 16 | AtPLDdb | Metabolism | rd29 | T1 | 487 |
| 17 | GmBRX | Lateral root | ABRC3 | T1 | 504 |
| 18 | AtMini3 | TF | ABRC3 | T1 | 505 |
| 19 | SlASR1 (Sl = Tomato) | Stomatal control | ABRC3 | T2 | 512 |
| 20 | AtABF3 | Stomatal control, water regulation | ABRC3 | T2 | 513 |
| 21 | GmATEM6 | LEA protein | ABRC3 | T1 | 506 |
| 22 | GmExp1 | Metabolism (cell wall loosening) | ABRC3 | T1 | 507 |
| 23 | GmPM30 (HVA1) | LEA protein | ABRC3 | T1 | 514 |
| 24 | GmZPTs | TF | 35S | T1 | 515 |
| 25 | GmZF-HD | TF | 35S | T0 | 516 |
| 26 | Gm bZIP (TC229348) | TF | 35S | T0 | 517 |

Group III - Drought responsive and root related genes ALREADY CLONED and the constructs are ready for plant transformation

| | | | | |
|---|---|---|---|---|
| 1 | Gm lsoflavone reductase (GmIFR) | Metabolism | 35S | 508 |
| 2 | Gm Chalcone synthase (GmCHS) | Metabolism | 35S | 509 |
| 3 | GmMyb | TF | 35S | 510 |

Group IV - Drought responsive and root related genes in the CLONING PIPELINE

| | | | | |
|---|---|---|---|---|
| 7 | GmNAC15 | TF | rd29, sub2 | 518 |
| 10 | GmNAC31 | TF | rd29, sub2 | 519 |
| 13 | GmHDSTART1 | TF | rd29, sub2 | 520 |
| 14 | GmHDSTART2 | TF | rd29, sub2 | 521 |
| 15 | GmHDSTART3 | TF | rd29, sub2 | 522 |
| 16 | GmHDSTART4 | TF | rd29, sub2 | 523 |
| 17 | GmHDSTART6 | TF | rd29, sub2 | 524 |
| 18 | GmHDSTART7 | TF | rd29, sub2 | 525 |
| 19 | GmHDSTART8 | TF | rd29, sub2 | 526 |
| 20 | GmHDSTART9 | TF | rd29, sub2 | 527 |
| 21 | GmHDSTART10 | TF | rd29, sub2 | 528 |
| 22 | GmHDSTART11 | TF | rd29, sub2 | 529 |
| 34 | GmLBD members | Lateral root | 35S | 530 |
| 47 | Gm Caffaeyl Co-A methyltransferase (CCoAOMT) | Metabolism | 35S | 531 |
| 48 | GmDREB2 | TF | 35S | 532 |
| 49 | GmbHLH (S4891443) | TF | 35S | 533 |
| 50 | GmbHLH (S4897794) | TF | | 534 |
| 51 | Gm beta Glucosidase (equiv to AtBG1) | ABA metabolism, wall loosening | 35S | 535 |
| 52 | GmNADPH oxidase | ROS signalling | Use RNAi | 536 |
| 44 | GmProton trasporter/Acquaporin | Metabolism | 35S | 537 |

Gm—Glycine max
At—Arabidopsis thaliana
Le—Lycopersicum esculentum
TF—Transcription factor
35S—CaMV35S promoter
rd29—dehydration inducible promoter
ABRC3—ABA inducible promoter
sub2—suberin
T1, T2, T3—Transgenic generation

1-26. (canceled)

27. A method of increasing drought tolerance of a plant, said method comprising:
(a) introducing into the plant genome a Root Specific Drought Response Gene (RSDRG) or a fragment thereof; and
(b) expressing said RSDRG or fragment thereof in said plant,
wherein said RSDRG or fragment thereof encodes a protein selected from the group consisting of a LEA protein, a signaling factor, a transcription factor, a metabolism-related protein, and a xyloglucan endo-transglycosylase.

28. The method of claim 27, wherein said RSDRG or fragment thereof encodes a LEA protein and comprises a nucleic acid sequence that is at least 95% identical to SEQ ID NO: 487 or SEQ ID NO: 514.

29. The method of claim 27, wherein said RSDRG or fragment thereof encodes a transcription factor and comprises a nucleic acid sequence that is at least 95% identical to SEQ ID NO: 492, 493, 494, 495, 496, 497, 498, 499, 500, 501, 502, 516, 517, 521 or 522.

30. The method of claim 27, wherein said RSDRG is operably linked to a promoter for regulating expression thereof.

31. The method of claim 30, wherein said promoter is selected from the group consisting of a cell specific promoter, a tissue specific promoter, an organ specific promoter, a constitutive promoter, and an inducible promoter.

32. The method of claim 27, wherein said plant is selected from the group consisting of soybean, corn, wheat, rice, cotton, and Arabidopsis.

33. A method of increasing drought tolerance of a plant, said method comprising altering expression level of a Root Specific Drought Response Gene (RSDRG) in said plant, said RSDRG being endogenous to said plant and encoding a protein selected from the group consisting of a LEA protein, a signaling factor, a transcription factor, a metabolism-related protein, and a xyloglucan endo-transglycosylase.

34. The method of claim 33, wherein said expression level is altered by modifying transcription regulation of said RSDRG.

35. The method of claim 33, wherein said plant is selected from the group consisting of soybean, corn, wheat, rice, cotton, and Arabidopsis.

36. A transgenic plant generated from a host plant and exhibiting increased drought tolerance as compared to the host plant, wherein said transgenic plant comprises a Root Specific Drought Response Gene (RSDRG) encoding a protein selected from the group consisting of a LEA protein, a signaling factor, a transcription factor, a metabolism-related protein, and a xyloglucan endo-transglycosylase, said RSDRG being derived from a plant that is genetically different from the host plant.

37. The transgenic plant of claim 36, exhibiting one or more altered phenotype selected from early maturation, increased growth rate, increased biomass, and increased lipid content.

38. The transgenic plant of claim 36, wherein said RSDRG encodes a LEA protein and comprises a nucleic acid sequence that is at least 95% identical to SEQ ID NO: 487 or SEQ ID NO: 514.

39. The transgenic plant of claim 36, wherein said RSDRG encodes a transcription factor and comprises a nucleic acid sequence that is at least 95% identical to SEQ ID NO: 492, 493, 494, 495, 496, 497, 498, 499, 500, 501, 502, 516, 517, 521 or 522.

40. The transgenic plant of claim 36, wherein said RSDRG is operably linked to a promoter for regulating expression thereof.

41. The transgenic plant of claim 40, wherein said promoter is selected from the group consisting of a cell specific promoter, a tissue specific promoter, an organ specific promoter, a constitutive promoter, and an inducible promoter.

42. The transgenic plant of claim 36, wherein said plant is selected from the group consisting of soybean, corn, wheat, rice, cotton, and Arabidopsis.

43. A genetically altered plant exhibiting increased drought tolerance as compared to a parental plant, wherein said genetically altered plant comprises altered expression level of a Root Specific Drought Response Gene (RSDRG), said RSDRG being endogenous to said plant and encoding a protein selected from the group consisting of a LEA protein, a signaling factor, a transcription factor, a metabolism-related protein, and a xyloglucan endo-transglycosylase.

44. The genetically altered plant of claim 43, wherein said expression level is altered by modifying transcription regulation of said RSDRG.

45. The genetically altered plant of claim 43, wherein said expression level in said genetically altered plant is at least 50% higher or lower than expression level in the parental plant.

46. The genetically altered plant of claim 43, wherein said plant is selected from the group consisting of soybean, corn, wheat, rice, cotton, and Arabidopsis.
